(19) United States
(12) Patent Application Publication
(10) Pub. No.: US 2021/0198256 A1
(43) Pub. Date: Jul. 1, 2021

(54) COMPOUNDS FOR THE DEGRADATION OF BRD9 OR MTH1

(71) Applicant: C4 Therapeutics, Inc., Watertown, MA (US)

(72) Inventors: Christopher G. Nasveschuk, Stoneham, MA (US); Ning Yin, Lexington, MA (US); Katrina L. Jackson, Weston, MA (US); Gesine Kerstin Veits, Sommerville, MA (US); Moses Moustakim, Brighton, MA (US); Jeremy L. Yap, Sudbury, MA (US); Rhamy Zeid, Arlington, MA (US)

(73) Assignee: C4 Therapeutics, Inc., Watertown, MA (US)

(21) Appl. No.: 17/192,634

(22) Filed: Mar. 4, 2021

Related U.S. Application Data
(63) Continuation of application No. PCT/US2019/049582, filed on Sep. 4, 2019.
(60) Provisional application No. 62/779,319, filed on Dec. 13, 2018.
(60) Provisional application No. 62/726,667, filed on Sep. 4, 2018.

(51) Int. Cl. C07D 471/04; C07D 401/14; C07D 417/14

(52) U.S. Cl. CPC C07D 471/04; C07D 417/14; C07D 401/14

(57) ABSTRACT

Compounds that degrade BRD9 or MTH1 via the ubiquitin proteasome pathway in a subject in need thereof for therapeutic applications are provided. The compounds provided have an E3 Ubiquitin Ligase targeting moiety (Degron) that is linked to a Targeting Ligand for BRD9 or MTH1.

### CROSS-REFERENCE TO RELATED APPLICATIONS

This application is a continuation of International Application No. PCT/US2019/049582, filed in the U.S. Receiving Office on Sep. 4, 2019, which claims the benefit of U.S. Provisional Patent Application No. 62/779,319, filed Dec. 13, 2018, and U.S. Provisional Patent Application No. 62/726,667, filed Sep. 4, 2018. The entirety of each of these applications is incorporated herein for all purposes.

### FIELD OF THE INVENTION

The invention provides compounds that degrade BRD9 or MTH1 by the ubiquitin proteasome pathway for therapeutic applications. The compounds of the present invention have an E3 Ubiquitin Ligase targeting moiety (Degron) that is linked to a Targeting Ligand for BRD9 or MTH1.

### BACKGROUND

Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection.

Covalent attachment of multiple ubiquitin molecules by an E3 ubiquitin ligase to terminal lysine residues marks the protein for proteasome degradation, where the protein is digested into small peptides and eventually into its constituent amino acids that serve as building blocks for new proteins. Defective proteasomal degradation has been linked to a variety of clinical disorders including Alzheimer's disease, Parkinson's disease, Huntington's disease, muscular dystrophies, cardiovascular disease, and cancer among others.

The drug thalidomide and its analogs lenalidomide and pomalidomide have garnered interest as immunomodulators and antineoplastics, especially in multiple myeloma (see Martiniani, R. et al. “Biological activity of lenalidomide and its underlying therapeutic effects in multiple myeloma” Adv Hematol, 2012, 2012:842945; and Terpos, E. et al. “Pomalidomide: a novel drug to treat relapsed and refractory multiple myeloma” Oncotargets and Therapy, 2013, 6:531). While the exact therapeutic mechanism of action of thalidomide, lenalidomide and pomalidomide is unknown, the compounds are used in the treatment of some cancers including multiple myeloma. There are also clinical and preclinical studies related to the treatment of renal cell carcinoma, glioblastoma, prostate cancer, melanoma, colorectal cancer, crohns disease, rheumatoid arthritis, Behcet's syndrome, breast cancer, head and neck cancer, ovarian cancer, chronic heart failure, graft-versus-host disease, and tuberculous meningitis.

Thalidomide and its analogues have been found to bind to the ubiquitin ligase cereblon and redirect its ubiquitination activity (see Ito, T. et al. “Identification of a primary target of thalidomide teratogenicity” Science, 2010, 327:1345). Cereblon forms part of an E3 ubiquitin ligase complex which interacts with damaged DNA binding protein 1, forming an E3 ubiquitin ligase complex with Cullin 4 and the E2-binding protein ROC1 (known as RBX1) where it functions as a substrate receptor to select proteins for ubiquitination. The binding of lenalidomide to cereblon facilitates subsequent binding of cereblon to Ikaros and Aiolos, leading to their ubiquitination and degradation by the proteasome (see Lu, G. et al. “The myeloma drug lenalidomide promotes the cereblon-dependent destruction of Ikaros proteins” Science, 2014, 343:305-309; Krönke, J. et al. “Lenalidomide causes selective degradation of IKZF1 and IKZF3 in multiple myeloma cells” Science, 2014, 343:301-305).

The disclosure that thalidomide binds to the cereblon E3 ubiquitin ligase led to research to investigate incorporating thalidomide and certain derivatives into compounds for the targeted destruction of proteins. Celgene has disclosed imids for similar uses, including those in U.S. Pat. Nos. 6,045,501; 6,315,720; 6,395,754; 6,561,976; 6,561,977; 6,755,784; 6,869,399; 6,908,432; 7,141,018; 7,230,012; 7,820,697; 7,874,984; 7,959,566; 8,204,763; 8,315,886; 8,589,188; 8,626,531; 8,673,939; 8,735,428; 8,741,929; 8,828,427; 9,056,120; 9,101,621; and 9,101,622. The Regents of the University of Michigan have also filed patent applications that disclose imids for the treatment of diseases, including WO 2017/176958 titled “Monofunctional Intermediates for Ligand-Dependent Target Protein Degradation”, which describes compounds that function as immunomodulators or monofunctional synthetic intermediates to prepare small-molecule drug conjugates for protein degradation. WO 2017/176957 and WO 2017/180417 which are also assigned to the Regents of the University of Michigan describe specific small-molecule protein degraders.

Patent applications have been filed in this area that use the ability of cereblon to direct degradation to targeted proteins by attaching a cereblon ligand and a protein targeting ligand with a covalent linker. WO 2016/105518 and WO 2017/007612 titled “Methods to Induce Targeted Protein Degradation Through Bifunctional Molecules” are assigned to Dana-Farber Cancer Institute and describe compounds capable of binding to an E3 ubiquitin ligase and a target protein for degradation. WO 2017/223452 titled “Degradatino of Bromodomain-Containing Protein 9 (BRD9) by Conjugation of BRD9 Inhibitors with E3 Ligase Ligand and Methods of Use” describes compounds capable of binding to an E3 ubiquitin ligase and BRD9.

Patent applications filed by C4 Therapeutics, Inc., that describe compounds capable of binding to an E3 ubiquitin ligase and a target protein for degradation include: WO 2017/197051 titled “Amine-Linked C3-Glutarimide Degronimers for Target Protein Degradation”; WO 2017/197055 titled “Heterocyclic Degronimers for Target Protein Degradation”; WO 2017/197036 titled “Spirocyclic Degronimers for Target Protein Degradation”; WO 2017/197046 titled “C3-Carbon Linked Glutarimide Degronimers for Target Protein Degradation”; WO 2017/197056 titled “Bromodomain Targeting Degronimers for Target Protein Degradation”; WO 2018/237026 titled “N/O-Linked Degrons and Degronimers for Protein Degradation”; and WO 2019/099868 titleed “Degraders and Degrons for Targeted Protein Degradation.”

Patent applications filed by C4 Therapeutics, Inc. and Hoffman-La Roche Inc. that describe compounds capable of binding to an E3 ubiquitin ligase and a target protein for degradation include: WO 2018/115218 titled “2-Benzopyrazinyl-N-heteroaryl-2-phenyl-acetamide Compounds”; WO 2019/121562 titled “Bifunctional Inhibitors with EGFR Having A E3 Ubiquitin Ligase Moiety”; and WO 2019/149922 titled “Compounds which Cause Degradation of EGFR, for use Against Cancer.”

Arvinas, Inc. has filed a patent application that describes compounds comprising a protein degradation moiety covalently bound to a linker and a targeting ligand, U.S. Patent Publication No. 2015/0291562 assigned to Arvinas, Inc. and titled “Imide-Based Modulators of Proteolysis and Associated Methods of Use.” In particular, the specification discloses protein degrading compounds that incorporate certain small molecules that can bind to an E3 ubiquitin ligase. Other patent applications filed by Arvinas that describe protein degrading compounds include: WO 2015/160845; WO 2016/118666; WO 2016/149668; WO 2016/197032; WO 2016/197114; WO 2017/030814; WO 2017/176708; WO 2018/053354; WO 2018/071606; WO 2018/102067; WO 2018/102725; WO 2018/118598; WO 2018/119357; WO 2018/119441; WO 2018/140809; WO 2018/144649; WO 2018/226542; and WO 2019/099926.

Foghorn Therapeutics Inc. has filed a patent application that describes compounds comprising a protein degradation moiety covalentaly bound to a linker and a BRD9 targeting ligand, WO 2019/152440 titled “Methods and Compounds for Treating Disorders.”

It is an object of the present invention to provide new compounds, methods, and compositions that are useful for the treatment of cancers and abnormal cellular proliferation.

### SUMMARY OF THE INVENTION

New compounds are provided for the treatment of cancer, abnormal cellular proliferation, and other indications as disclosed herein, along with their uses and manufacture, that degrade BRD9 or MTH1 via the ubiquitin proteasome pathway (UPP). The compounds of the present invention comprise an E3 Ubiquitin Ligase targeting moiety (Degron) that is linked to a Targeting Ligand for BRD9 or MTH1.

BRD9 is a protein that contains a bromodomain, which is a protein that recognizes acetylated lysine residues such as those on the N-terminals of histones. Bromodomain containing proteins have a number of functions that relate to transcription mediation and coactivation, therefore, they are involved in cellular proliferation.

2-Hydroxy-dATP diphosphatase which is also known as Nudix hydrolase 1 (NUDT1) or MutT homolog 1 (MTH1) is an enzyme that in humans is encoded by the NUDT1 gene. During DNA repair this enzyme hydrolyses oxidized purines and prevents their addition on the DNA chain. Thus, because of the important role of DNA synthesis in cancer MTH1 is a target for next generation chemotherapy.

A selected compound disclosed herein, its pharmaceutically acceptable salt, or its pharmaceutically acceptable composition can be used to treat a disorder mediated by BRD9 or MTH1, for example, a hematopoietic malignancy such as Hodgkin's lymphoma or Non-Hodgkin's lymphoma, NUT midline carcinoma, or leukemia. Therefore, in some embodiments a method to treat a host (typically a human) with a disorder mediated by BRD9 or MTH1, is provided that includes administering an effective amount of the disclosed compound or its pharmaceutically acceptable salt described herein to the host, optionally as a pharmaceutically acceptable composition.

The selected compound disclosed herein is also useful in the administration of chimeric antigen receptor T-cell therapy (CAR-T therapy), where the CAR-T cell is engineered to have a BRD9 or MTH1 off switch, in other words, the cell includes a BRD9 or MTH1 protein or fragment that binds to a ligand in a molecule provided herein.

The compounds of Formula I, Formula II, Formula III, and Formula IV bind and degrade BRD9. The compounds of Formula V and Formula VI bind and degrade MTH1.

In one aspect, the compound of the present invention is a BRD9-binding compound selected from Formula I, Formula II, or Formula III:

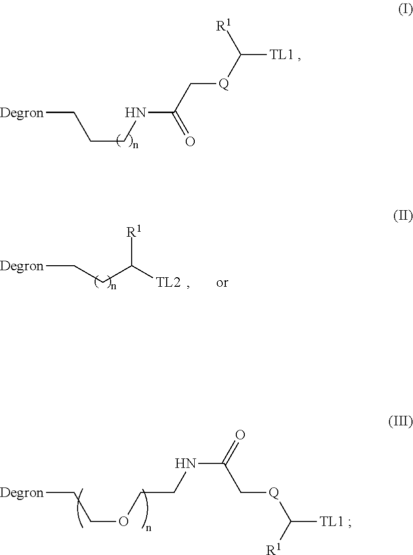

or a pharmaceutically acceptable salt, N-oxide, isotopic derivative, or prodrug thereof, optionally in a pharmaceutically acceptable carrier to form a composition;

wherein:

Degron is selected from:

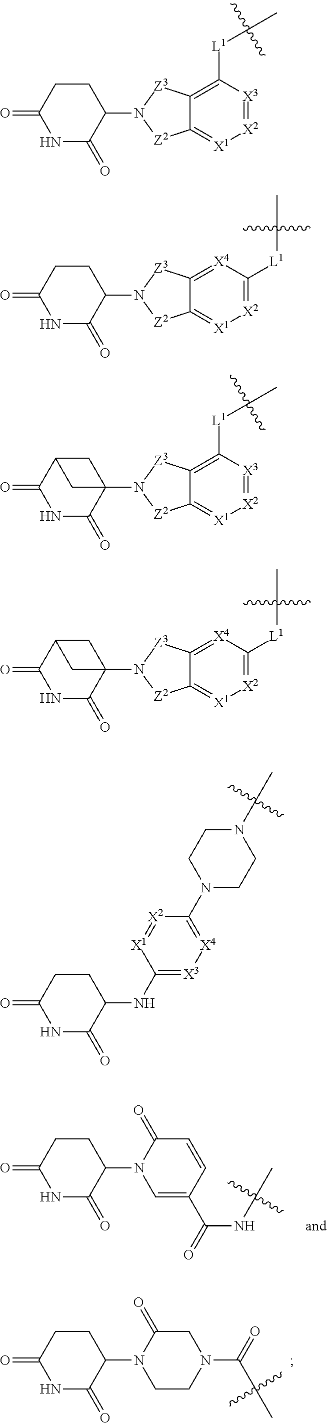

TL1 is a moiety that binds to BRD9 selected from

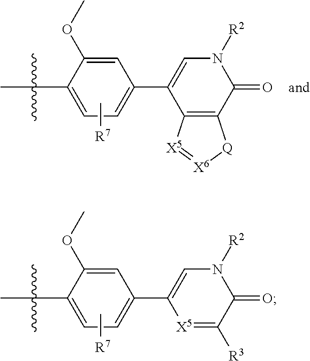

TL2 is a moiety that binds to BRD9 selected from

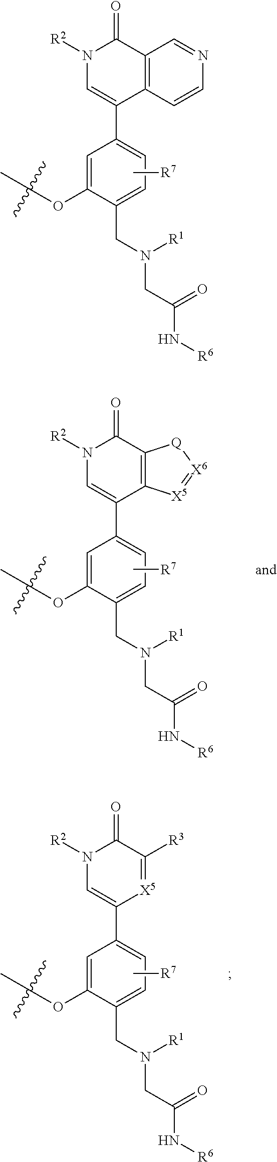

L¹ is selected from:

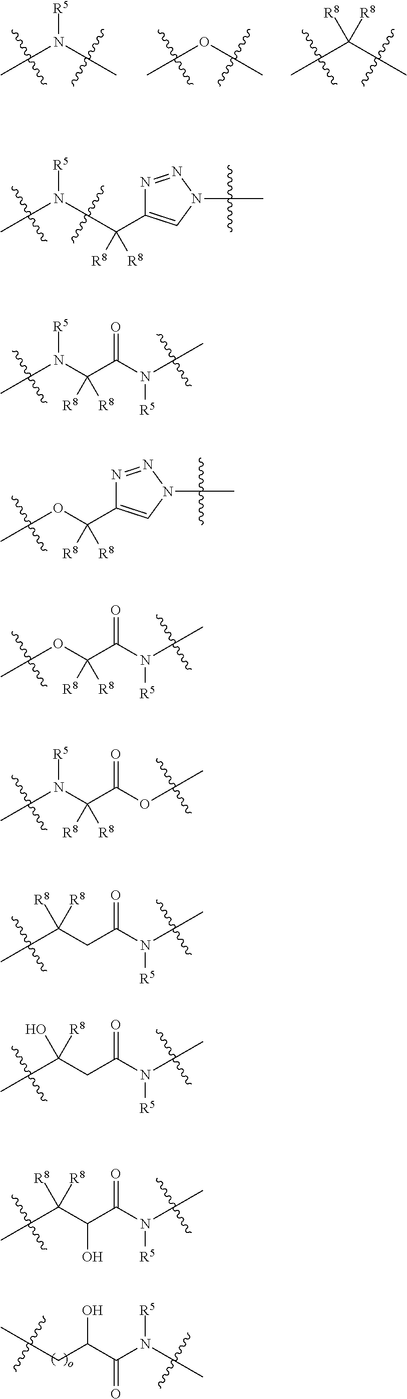

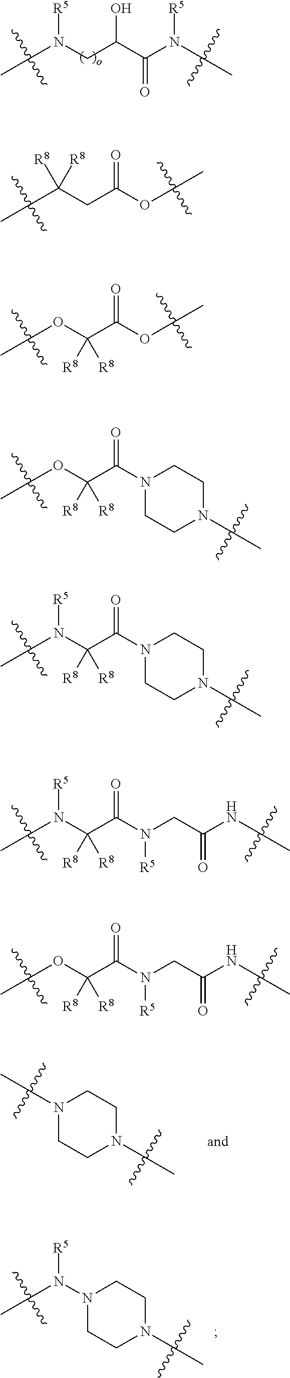

X¹, X², X³, and X⁴ are independently selected from CR⁴ and N, wherein no more than two of X¹, X², X³, and X⁴ may be selected to be N;

X⁵ and X⁶ are independently selected from CR⁴ and N;

Z² and Z³ are selected from —CH₂— and —C(O)— wherein at least one of Z² and Z³ is —C(O)—;

n is 0, 1, 2, 3, 4, 5, 6, 7, 8, 9, or 10;

o is 1, 2, 3, or 4;

each Q is independently 0, S, or NR⁵;

R¹ is hydrogen or C₁-C₆ alkyl;

R², R³, and R⁶ are independently selected from hydrogen and C₁-C₆alkyl;

each R⁴ is independently selected from hydrogen, halogen, hydroxyl, C₁-C₆alkyl, C₁-C₆alkoxy, and C₁-C₆haloalkyl;

each R⁵ is independently hydrogen, C₁-C₆alkyl, or —C(O)alkyl;

R⁷ is selected from halogen, hydrogen, C₁-C₆alkyl, C₁-C₆alkoxy, and C₁-C₆haloalkyl; and

each R⁸ is independently selected from hydrogen, C₁-C₆alkyl, and C₁-C₆haloalkyl; or two R⁸ groups together with the carbon to which they are attached form a cyclopropyl group.

In one embodiment, the BRD9-binding moiety TL1 is selected from

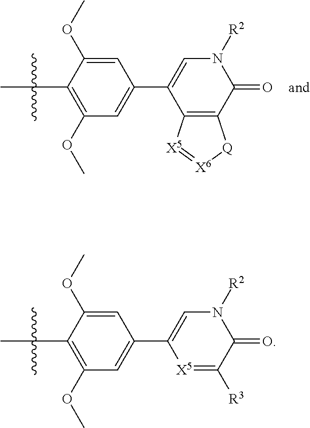

In another aspect, the compound of the present invention is a BRD9-binding compound selected from Formula IV:

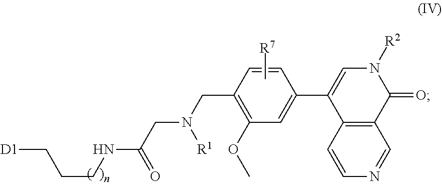

or a pharmaceutically acceptable salt, N-oxide, isotopic derivative, or prodrug thereof, optionally in a pharmaceutically acceptable carrier to form a composition;

wherein:

D1 is selected from:

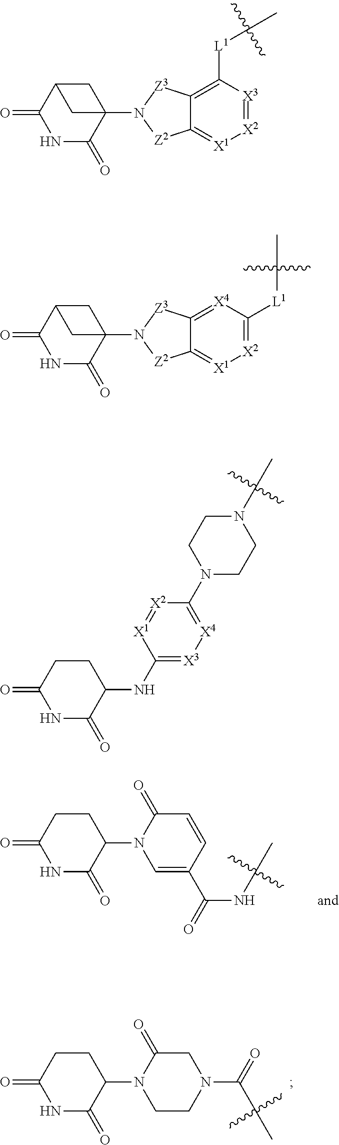

and all other variables are as defined herein.

In one embodiment, the compound of Formula I, Formula II, Formula III, or Formula IV is used to treat a disorder mediated by BRD9. In another embodiment, the compound is administered to a patient receiving CAR-T therapy to activate or deactivate the CAR-T cells by interacting with BRD9 or a protein fragment of BRD9 on the CAR-T cell. In another embodiment, the compound is administered to a patient receiving CAR-T therapy to deactivate the CAR-T cells by interacting with BRD9 or a protein fragment of BRD9 on the CAR-T cell.

Non-limiting examples of compounds of Formula I include:

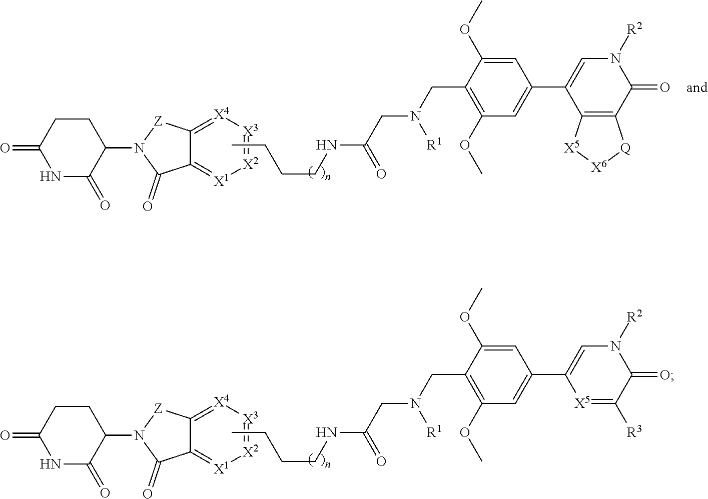

or a pharmaceutically acceptable salt thereof;

wherein

Z is CH₂ or C(O).

Additional non-limiting examples of compounds of Formula I include:

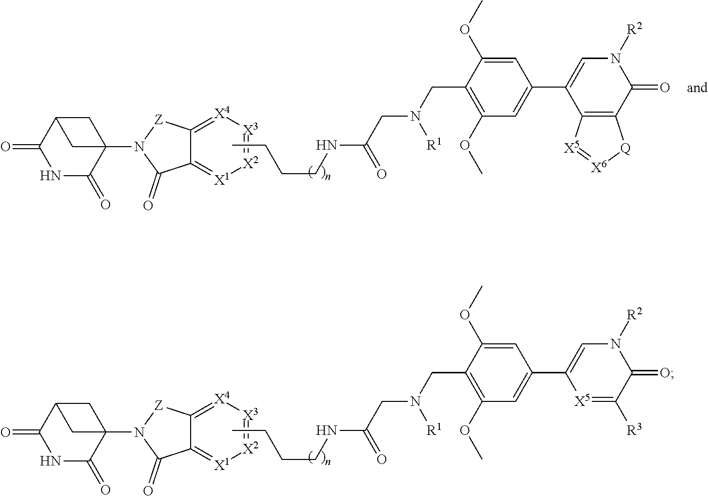

or a pharmaceutically acceptable salt thereof;

wherein

Z is CH₂ or C(O).

Additional non-limiting examples of compounds of Formula I include:

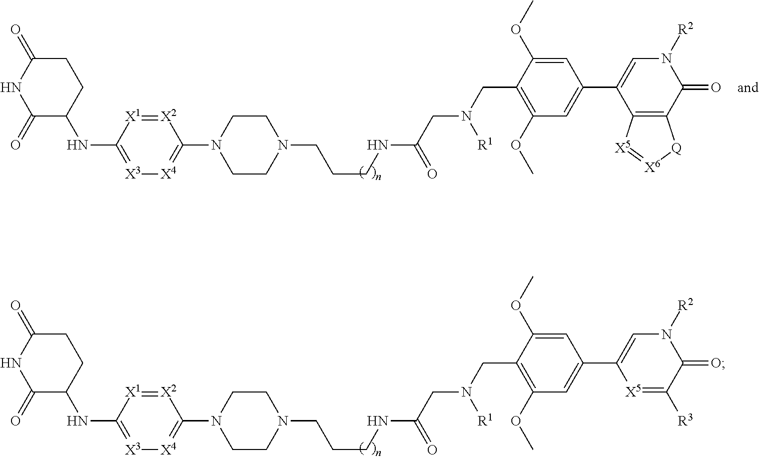

or a pharmaceutically acceptable salt thereof.

Additional non-limiting examples of compounds of Formula I include:

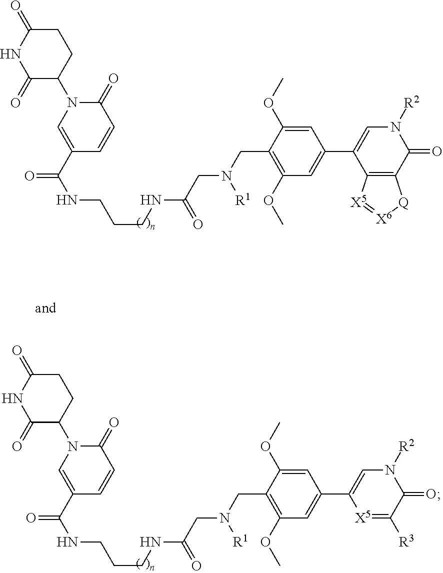

or a pharmaceutically acceptable salt thereof.

Additional non-limiting examples of compounds of Formula I include:

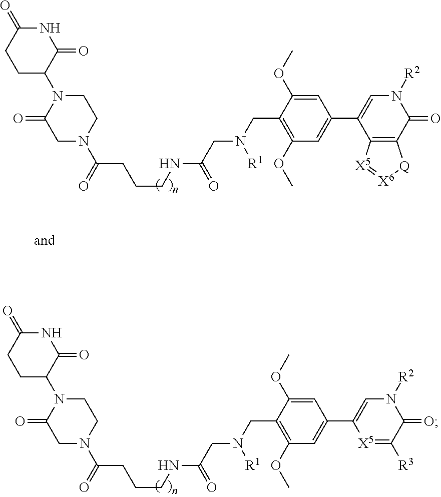

or a pharmaceutically acceptable salt thereof.

Non-limiting examples of compounds of Formula II include:

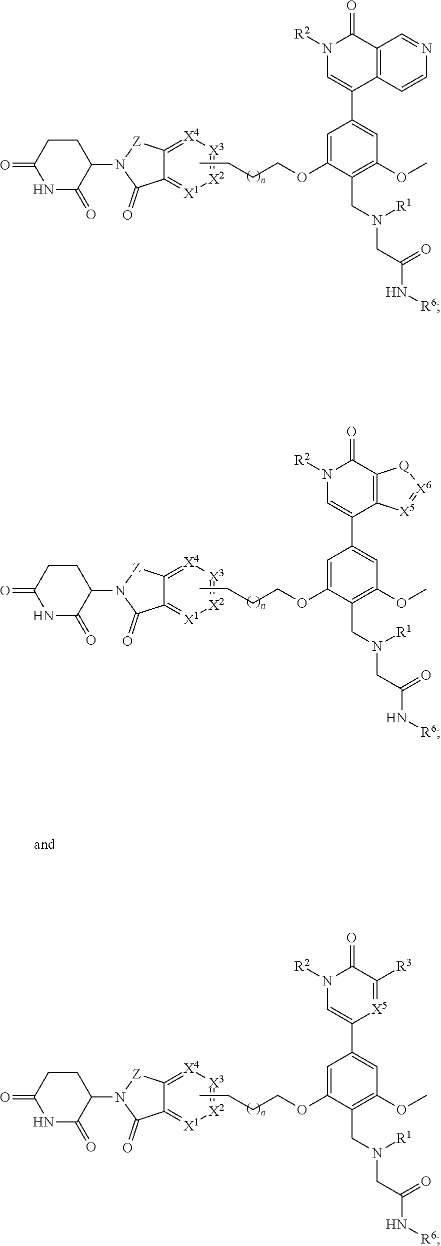

or a pharmaceutically acceptable salt thereof.

Additional non-limiting examples of compounds of Formula II include:

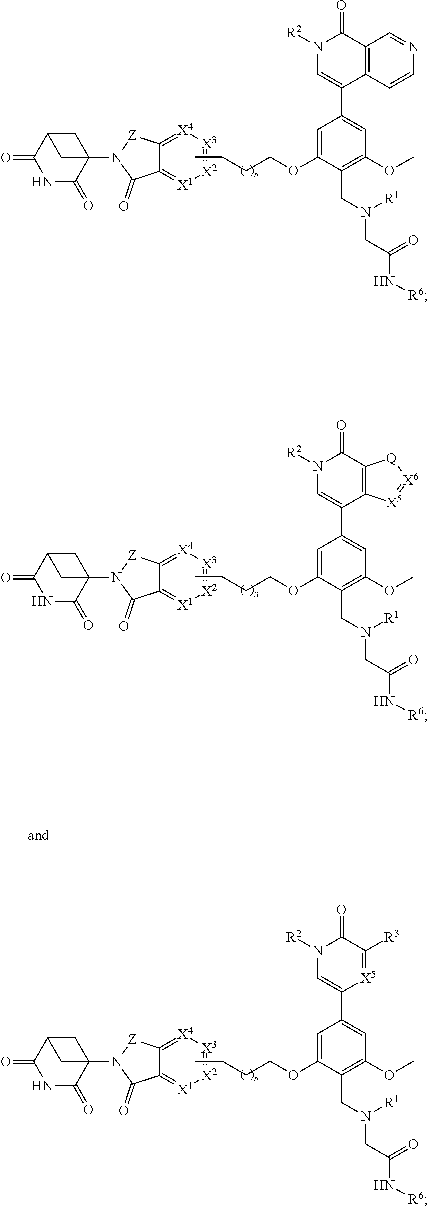

or a pharmaceutically acceptable salt thereof.

Additional non-limiting examples of compounds of Formula II include:

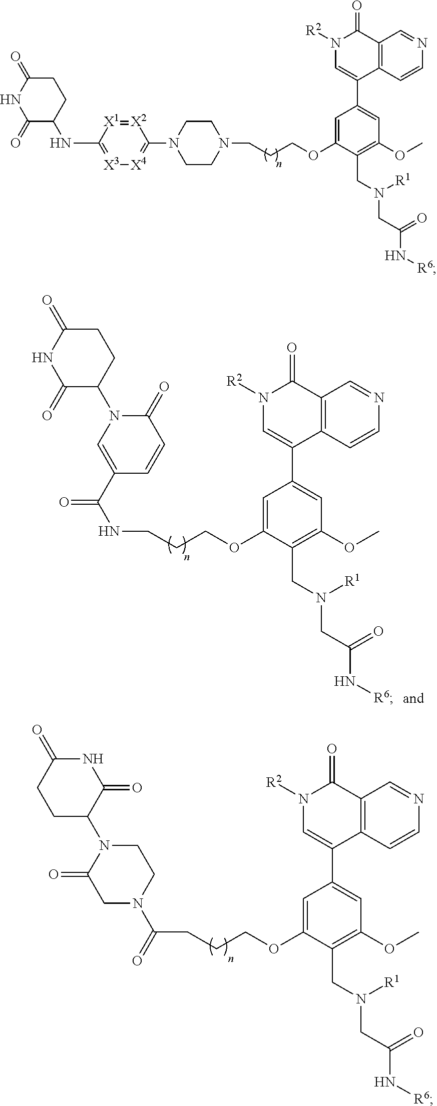

or a pharmaceutically acceptable salt thereof.

In another aspect, the compound of the present invention is a MTH1-binding compound selected from Formula V or Formula VI:

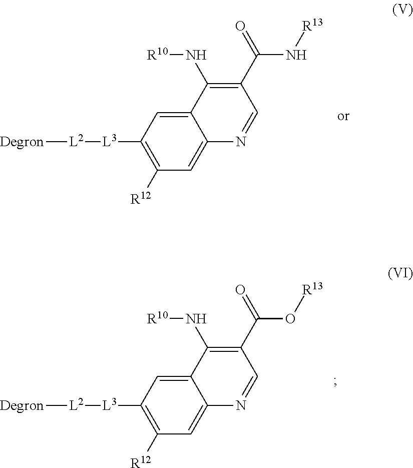

or a pharmaceutically acceptable salt, N-oxide, isotopic derivative, or prodrug thereof, optionally in a pharmaceutically acceptable carrier to form a composition;

L² is selected from: bond,

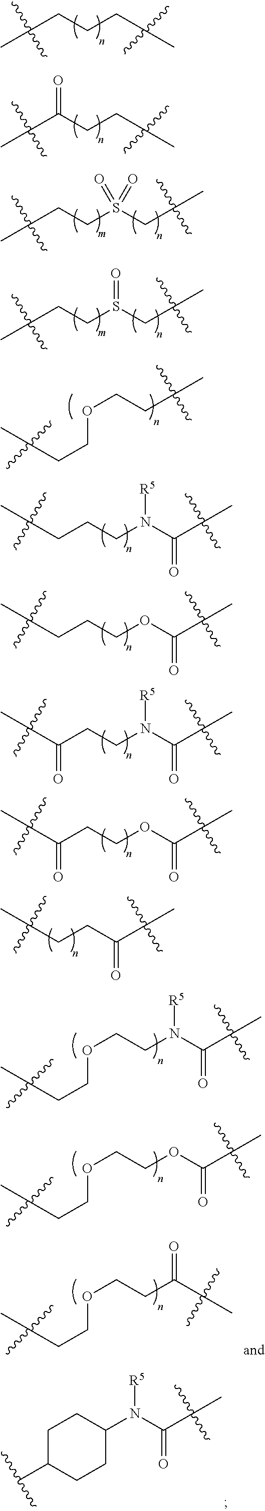

L³ is selected from bond, aryl, heterocycle, heteroaryl,

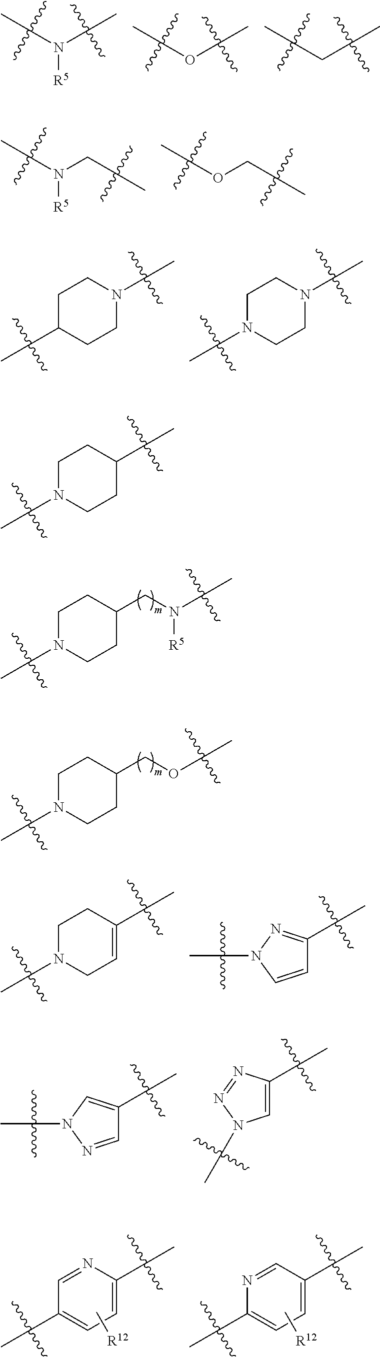

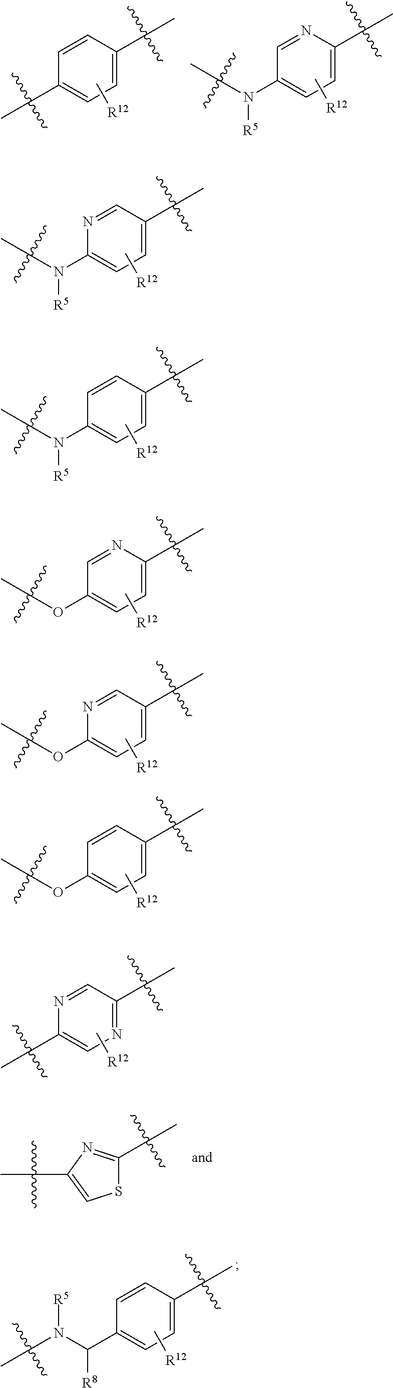

m is 0, 1, 2, 3, 4, 5, 6, 7, 8, 9, or 10;

R¹⁰ is selected from C₁-C₆alkyl, cycloalkyl, heterocycle, heteroaryl, —C₁-C₆alkyl-aryl, and aryl; each of which R¹⁰ group is optionally substituted with 1, 2, 3, or 4 substituents independently selected from R¹¹;

in an alternative embodiment, R¹⁰ is hydrogen;

R¹¹ is selected from hydrogen, halogen, —NR¹R¹⁴, —OR¹⁴, C₁-C₆alkyl, C₁-C₆haloalkyl, —SO₂NR¹R¹⁴, —SO₂OR¹⁴, —SONR¹R¹⁴, and —S(O)OR¹⁴;

each R² is independently selected from hydrogen, halogen, C₁-C₆alkyl, C₁-C₆alkoxy, and C₁-C₆haloalkyl;

R¹³ is selected from hydrogen, C₁-C₆alkyl, cycloalkyl, and heterocycle; each of which cycloalkyl and heterocycle is optionally substituted with 1, 2, 3, or 4 substituents independently selected from R¹¹;

each instance of R¹⁴ is independently selected from hydrogen, C₁-C₆alkyl, C(O)alkyl, and C(O)NR¹R¹;

and all other variables are as defined herein.

In one embodiment, the MTH1-binding compound of Formula V is selected from:

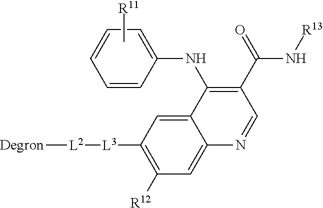

or a pharmaceutically acceptable salt, N-oxide, isotopic derivative, or prodrug thereof, optionally in a pharmaceutically acceptable carrier to form a composition.

In one embodiment, L²-L³ is selected from

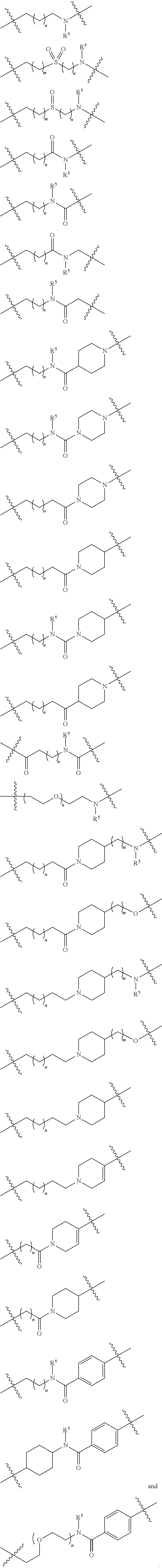

Non-limiting examples of compounds of MTH1-binding compounds of Formula V include:

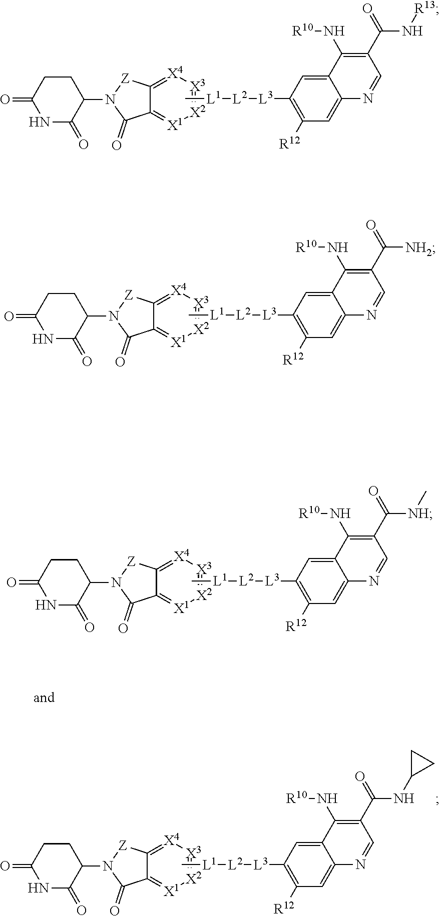

or a pharmaceutically acceptable salt thereof.

Additional non-limiting examples of compounds of Formula V include:

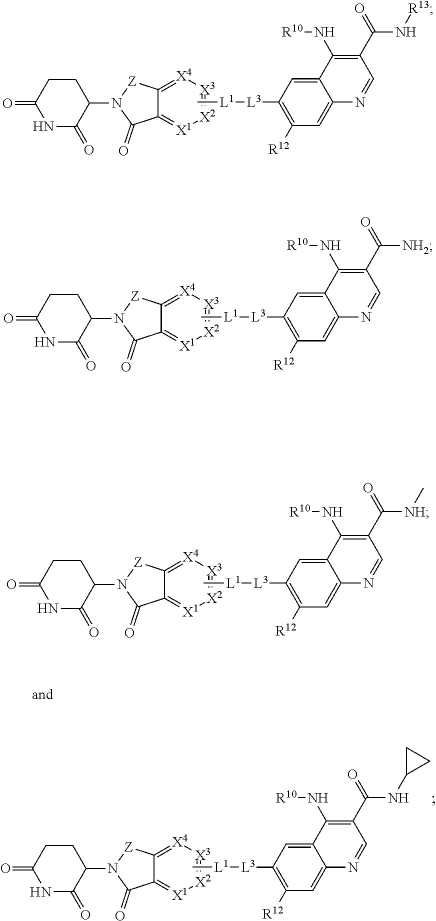

or a pharmaceutically acceptable salt thereof.

For clarity when a floating bond is drawn on a ring it can be attached at any position in the ring and it replaces the unselected variable to which it attaches. For example

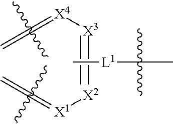

represents

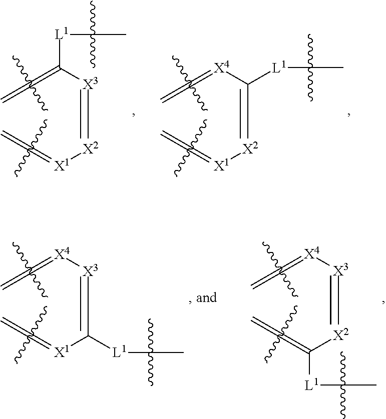

and each moiety is considered specifically described.
Thus, the present invention includes at least the following features:
a. a selected compound as described herein, or a pharmaceutically acceptable salt, isotopic derivative (including the deuterated derivative), or prodrug thereof;
b. a method of treating a disorder mediated by MTH1 or BRD9 as described further herein comprising administering an effective amount of a compound of the present invention or a pharmaceutically acceptable salt, isotopic derivative (including the deuterated derivative), or prodrug thereof to a patient;
c. use of a compound as described herein in an effective amount in the treatment of a patient, typically a human, with a disorder mediated by MTH1 or BRD9;
d. use of a compound as described herein or a pharmaceutically acceptable salt, isotopic derivative (including a deuterated derivative), or prodrug thereof in the manufacture of a medicament for the treatment of a medical disorder that can be alleviated, modified, or cured by the compound, as further described herein;
e. a method of deactivating CAR-T cells comprising administering an effective amount of a compound of the present invention or a pharmaceutically acceptable salt, isotopic derivative (including the deuterated derivative), or prodrug thereof to a patient;
f. use of a compound as described herein or a pharmaceutically acceptable salt, isotopic derivative (including a deuterated derivative), or prodrug thereof to deactivate CAR-T cells that have a CAR ligand with a MTH1 or BRD9 protein or fragment attached;
g. use of a compound as described herein or a pharmaceutically acceptable salt, isotopic derivative (including a deuterated derivative), or prodrug thereof in the manufacture of a medicament for deactivating CAR-T cells that have a CAR ligand with a MTH1 or BRD9 protein or fragment attached;
h. a pharmaceutical composition comprising an effective host-treating amount of a compound as described herein or a pharmaceutically acceptable salt, isotopic derivative, or prodrug thereof with a pharmaceutically acceptable carrier or diluent;
i. use of a compound as described herein or a pharmaceutically acceptable salt, isotopic derivative (including a deuterated derivative), or a prodrug thereof to modulate the activity of a T-cell expressing a chimeric antigen receptor (CAR), wherein the CAR comprises a MTH1 protein fragment or BRD9 protein fragment.
j. use of a compound as described herein or a pharmaceutically acceptable salt, isotopic derivative (including a deuterated derivative), or a prodrug thereof to in the manufacture of a medicate for modulating the activity of a T-cell expressing a chimeric antigen receptor (CAR), wherein the CAR comprises a MTH1 protein fragment or BRD9 protein fragment.
k. a compound as described herein as a mixture of enantiomers or diastereomers (as relevant), including as a racemate;
l. a compound as described herein in enantiomerically or diastereomerically (as relevant) enriched form, including an isolated enantiomer or diastereomer (i.e. greater than 85, 90, 95, 97, or 99% pure); and
m. a process for the preparation of therapeutic products that contain an effective amount of a compound as described herein.

### DETAILED DESCRIPTION OF THE INVENTION

### Definitions

Compounds are described using standard nomenclature. Unless defined otherwise, all technical and scientific terms used herein have the same meaning as is commonly understood by one of skill in the art to which this invention belongs.

The BRD9-binding compounds of Formula I, II, III, and IV and MTH1-binding compounds of Formula V and VI as described herein may be provided in the form of a racemate, enantiomer, mixture of enantiomers, diastereomer, mixtures of diastereomers, tautomer, N-oxide, an isomer such as a rotamer, as if each is specifically described, unless otherwise drawn or a designation is clear from the context herein.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The term “alkyl” is a branched or straight chain saturated aliphatic hydrocarbon group. In one non-limiting embodiment, the alkyl group contains from 1 to about 12 carbon atoms, more generally a lower alkyl from 1 to about 6 carbon atoms or from 1 to about 4 or 1 to about 3 carbon atoms. In one non-limiting embodiment, the alkyl contains from 1 to about 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane.

The term “alkyl” also encompasses cycloalkyl groups. For example, when a term is used that includes “alk” then “cycloalkyl” or “carbocyclic” can be considered part of the definition, unless unambiguously excluded by the context. For example and without limitation, the terms alkyl, alkoxy, haloalkyl, etc. can all be considered to include the cyclic forms of alkyl, unless unambiguously excluded by context.

“Halo” or “halogen” means —Cl, —Br, —I or —F (and typically F). In certain embodiments, “halo” or “halogen” may refers independently to —Cl or —F.

“Haloalkyl” is a branched or straight-chain alkyl group substituted with 1 or more halo atoms (typically F), up to the maximum allowable number of halogen atoms. In certain embodiments, the haloalkyl is C₁-C₂, C₁-C₃, C₁-C₄, C₁-C₅, or C₁-C₆. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more cycloalkyl or heterocycle groups wherein the radical or point of attachment is on the aryl ring, and in such instances, the number of carbon atoms continue to designate the number of carbon atoms in the aryl ring system. The one or more fused cycloalkyl or heterocycle groups can be 4 to 7-membered saturated or partially unsaturated cycloalkyl or heterocycle groups.

The term “heteroaryl” denotes stable aromatic ring systems that contain one or more heteroatoms selected from O, N, and S, wherein the ring nitrogen and sulfur atom(s) are optionally oxidized, and nitrogen atom(s) are optionally quaternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, IH-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. In one embodiment, the “heteroaryl” group is a 8, 9, or 10 membered bicyclic ring system. Examples of 8, 9, or 10 membered bicyclic heteroaryl groups include benzofurazanyl, benzothiophenyl, benzothiazolyl, benzoxazolyl, quinazolinyl, quinoxalinyl, naphthyridinyl, quinolinyl, isoquinolinyl, benzofuranyl, indolyl, indazolyl, and benzotriazolyl.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O—. —O—S—, or —S—S— portions. Examples of saturated heterocycle groups include saturated 4- to 7-membered monocyclic groups containing 1 to 4 nitrogen atoms [e.g. pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1V-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

A “prodrug” as used herein, means a compound which when administered to a host in vivo is converted into a parent drug. As used herein, the term “parent drug” means any of the presently described chemical compounds described herein. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Nonlimiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

As used herein, the term “MTH1 protein fragment” refers to an amino acid sequence derived from the human MTH1 protein (UniProtKB—P36639 (8ODP_HUMAN)), or variant thereof. The MTH1 protein fragment may include the full amino acid sequence of the MTH1 protein, or a partial amino acid sequence of the MTH1 protein, or variants thereof. In some embodiments, the MTH1 protein fragment comprises an amino acid sequence comprising 10, 15, 20, 25, 30, 35, 40, 45, 50 or more amino acids from the MTH1 protein.

As used herein, the term “BRD9 protein fragment” refers to an amino acid sequence derived from the human BRD9 protein (UnitProtKB-Q9H8M2 (BRD9-HUMAN)), or variant thereof. The BRD9 protein fragment may include the full amino acid sequence of the BRD9 protein, or a partial amino acid sequence of the BRD9 protein, or variants thereof. In some embodiments, the BRD9 protein fragment comprises an amino acid sequence comprising 10, 15, 20, 25, 30, 35, 40, 45, 50 or more amino acids from the BRD9 protein.

The present invention includes compounds of Formula I, II, III, IV, V, and VI with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, fluorine and chlorine such as ²H, ³H, ¹¹C, ¹³C, ¹⁴C, ¹⁵N, ¹⁷O, ¹⁸O, ¹⁸F, ³⁵S, ³⁶Cl, and respectively. In one non-limiting embodiment, isotopically labelled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In one non-limiting embodiment, the substitution of a hydrogen atom for a deuterium atom can be provided in a compound of Formula I, II, III, IV, V, or VI. In one non-limiting embodiment, the substitution of a hydrogen atom for a deuterium atom occurs within a group selected from any of Degron, X¹, X², X³, X⁴, Z², Z³, L¹, L², L³, TL1, TL2, X⁵, R⁶, n, o, Q, R¹, R², R³, R⁴, R⁵, R⁶, R⁷, R⁸, R¹⁰, R¹¹, R¹², and R¹³.

For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle the unsubstituted carbons may be deuterated.

### Compounds

### Embodiments of “alkyl”

In one embodiment, “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In one embodiment, “alkyl” has one carbon.

In one embodiment, “alkyl” has two carbons.

In one embodiment, “alkyl” has three carbons.

In one embodiment, “alkyl” has four carbons.

In one embodiment, “alkyl” has five carbons.

In one embodiment, “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

### Embodiments of “haloalkyl”

In one embodiment, “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In one embodiment, “haloalkyl” has one carbon.

In one embodiment, “haloalkyl” has one carbon and one halogen.

In one embodiment, “haloalkyl” has one carbon and two halogens.

In one embodiment, “haloalkyl” has one carbon and three halogens.

In one embodiment, “haloalkyl” has two carbons.

In one embodiment, “haloalkyl” has three carbons.

In one embodiment, “haloalkyl” has four carbons.

In one embodiment, “haloalkyl” has five carbons.

In one embodiment, “haloalkyl” has six carbons.

Non-limiting examples of “haloalkyl” include:

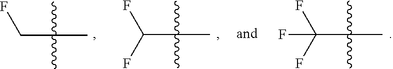

Additional non-limiting examples of “haloalkyl” include:

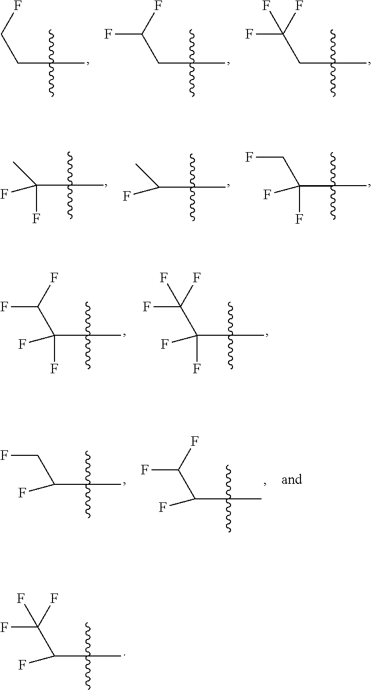

Additional non-limiting examples of “haloalkyl” include:

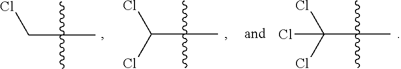

Additional non-limiting examples of “haloalkyl” include:

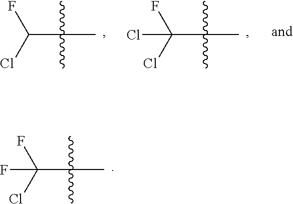

### Embodiments of “aryl”

In one embodiment, “aryl” is a 6 carbon aromatic group (phenyl)

In one embodiment, “aryl” is a 10 carbon aromatic group (napthyl)

In one embodiment, “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

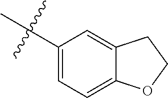 [SMILES: *c1ccc2c(c1)CCO2]

is an “aryl” group.

However,

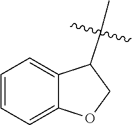 [SMILES: *C1COc2ccccc21]

is a “heterocycle” group.

In one embodiment, “aryl” is a 6 carbon aromatic group fused to a cycloalkyl wherein the point of attachment is the aryl ring. Non-limiting examples of “aryl” include dihydro-indene and tetrahydronaphthalene wherein the point of attachment for each group is on the aromatic ring.

For example

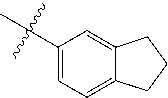 [SMILES: *c1ccc2c(c1)CCC2]

is an “aryl” group.

However,

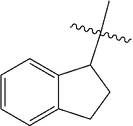 [SMILES: *C1CCc2ccccc21]

is a “cycloalkyl” group.

### Embodiments of “heteroaryl”

In one embodiment, “heteroaryl” is a 5 membered aromatic group containing 1, 2, 3, or 4 nitrogen atoms.

Non-limiting examples of 5 membered “heteroaryl” groups include pyrrole, furan, thiophene, pyrazole, imidazole, triazole, tetrazole, isoxazole, oxazole, oxadiazole, oxatriazole, isothiazole, thiazole, thiadiazole, and thiatriazole.

Additional non-limiting examples of 5 membered “heteroaryl” groups include:

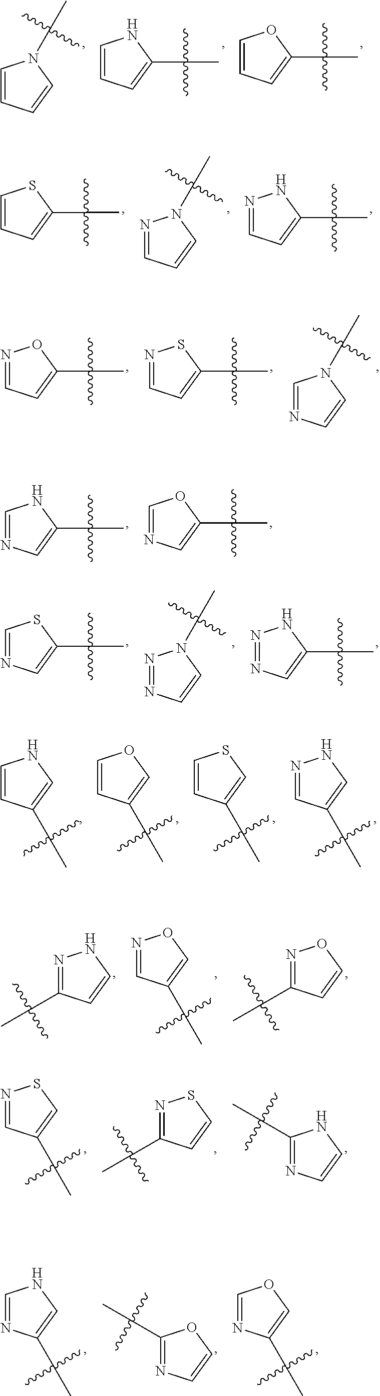

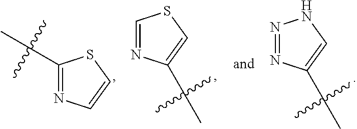

In one embodiment, “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

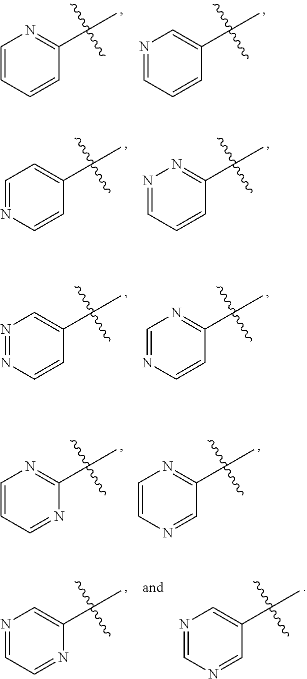

In one embodiment, “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

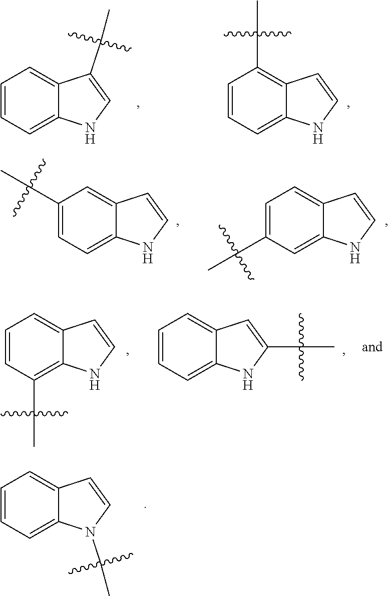

Additional non-limiting examples of “heteroaryl” groups that are bicyclic include:

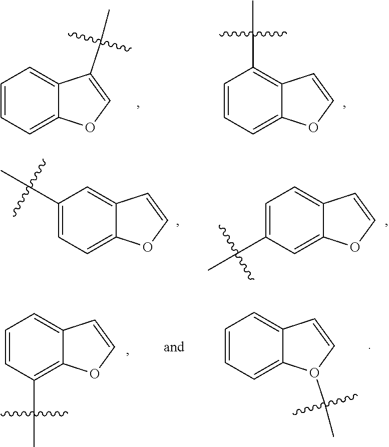

Additional non-limiting examples of “heteroaryl” groups that are bicyclic include:

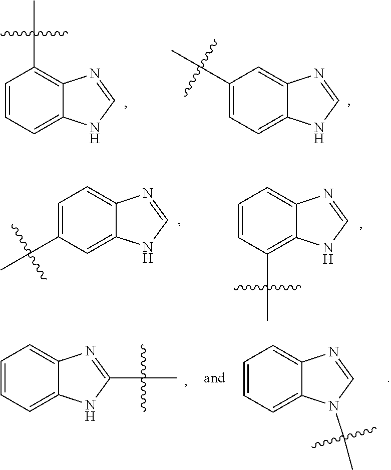 [SMILES: *c1cccc2[nH]cnc12 ; *c1ccc2[nH]cnc2c1 ; *c1ccc2nc[nH]c2c1 ; *c1cccc2nc[nH]c12 ; *c1nc2ccccc2[nH]1 ; *n1cnc2ccccc21]

In one embodiment, “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

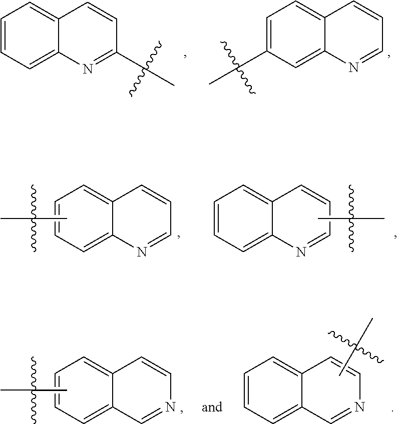

### Embodiments of “cycloalkyl”

In one embodiment, “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In one embodiment, “cycloalkyl” has three carbons.

In one embodiment, “cycloalkyl” has four carbons.

In one embodiment, “cycloalkyl” has five carbons.

In one embodiment, “cycloalkyl” has six carbons.

In one embodiment, “cycloalkyl” has seven carbons.

In one embodiment, “cycloalkyl” has eight carbons.

In one embodiment, “cycloalkyl” has nine carbons.

In one embodiment, “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

Additional non-limiting examples of “cycloalkyl” include dihydro-indene and tetrahydronaphthalene wherein the point of attachment for each group is on the cycloalkyl ring.

For example

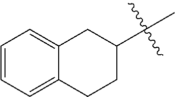 [SMILES: *C1CCc2ccccc2C1]

is an “cycloalkyl” group.

However,

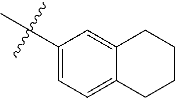 [SMILES: *c1ccc2c(c1)CCCC2]

is an “aryl” group.

Additional examples of “cycloalkyl” groups include

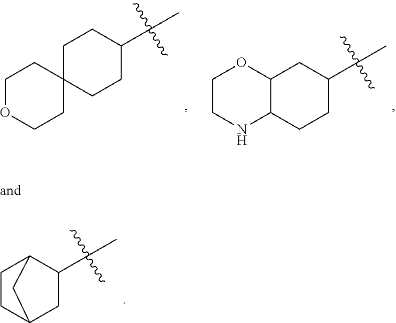 [SMILES: *C1CCC2(CCOCC2)CC1 ; *C1CCC2NCCOC2C1 ; *C1CC2CCC1C2]

### Embodiments of “heterocycle”

In one embodiment, “heterocycle” refers to a cyclic ring with one nitrogen and 3, 4, 5, 6, 7, or 8 carbon atoms.

In one embodiment, “heterocycle” refers to a cyclic ring with one nitrogen and one oxygen and 3, 4, 5, 6, 7, or 8 carbon atoms.

In one embodiment, “heterocycle” refers to a cyclic ring with two nitrogens and 3, 4, 5, 6, 7, or 8 carbon atoms.

In one embodiment, “heterocycle” refers to a cyclic ring with one oxygen and 3, 4, 5, 6, 7, or 8 carbon atoms.

In one embodiment, “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

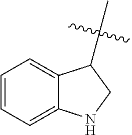 [SMILES: *C1CNc2ccccc21]

is a “heterocycle” group.

However,

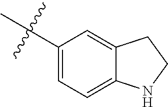 [SMILES: *c1ccc2c(c1)CCN2]

is an “aryl” group.

Non-limiting examples of “heterocycle” also include:

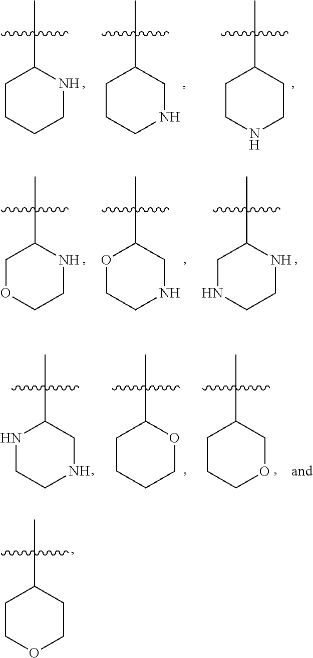

Additional non-limiting examples of “heterocycle” include:

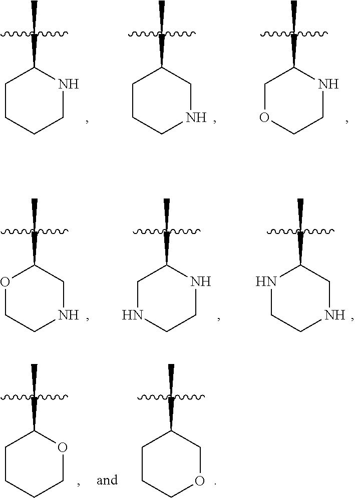

Additional non-limiting examples of “heterocycle” include:

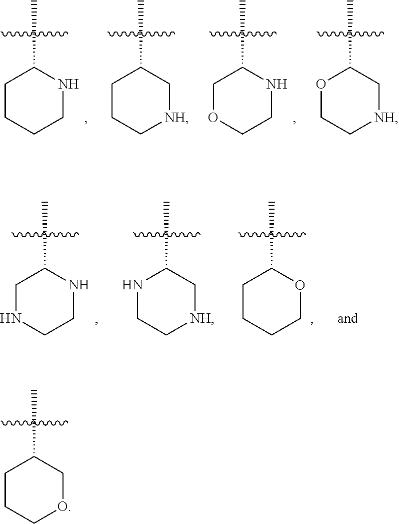

Non-limiting examples of “heterocycle” also include:

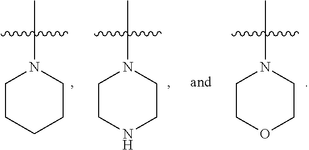

Non-limiting examples of “heterocycle” also include:

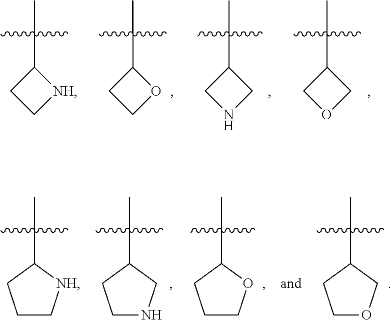

Additional non-limiting examples of “heterocycle” include:

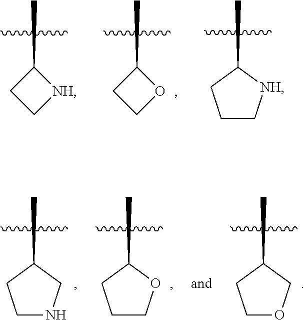

Additional non-limiting examples of “heterocycle” include:

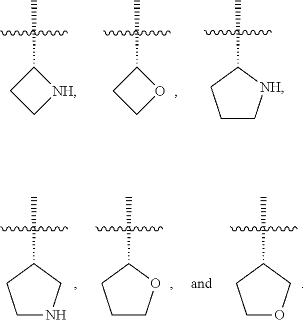

### Optional Substituents

In one embodiment, a group described herein that can be substituted with 1, 2, 3, or 4 substituents is substituted with one substituent.

In one embodiment, a group described herein that can be substituted with 1, 2, 3, or 4 substituents is substituted with two substituents.

In one embodiment, a group described herein that can be substituted with 1, 2, 3, or 4 substituents is substituted with three substituents.

In one embodiment, a group described herein that can be substituted with 1, 2, 3, or 4 substituents is substituted with four substituents.

### Additional Embodiments

In one embodiment, the BRD9-binding moiety TL1 is selected from:

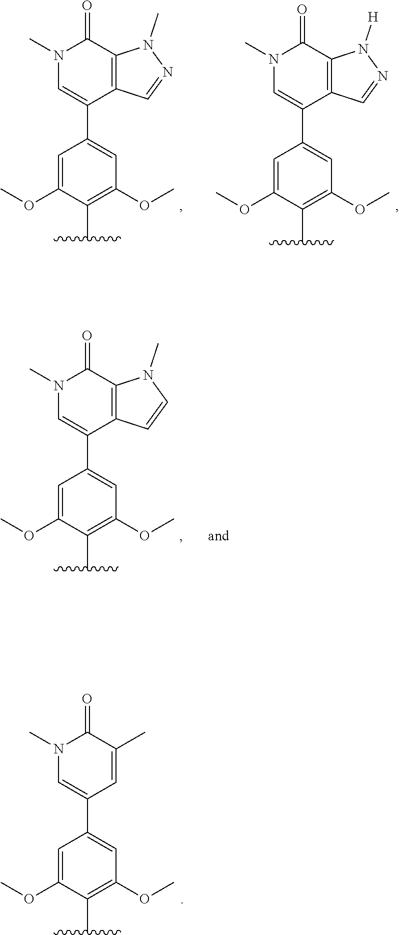

In one embodiment, the BRD9-binding moiety TL2 is selected from:

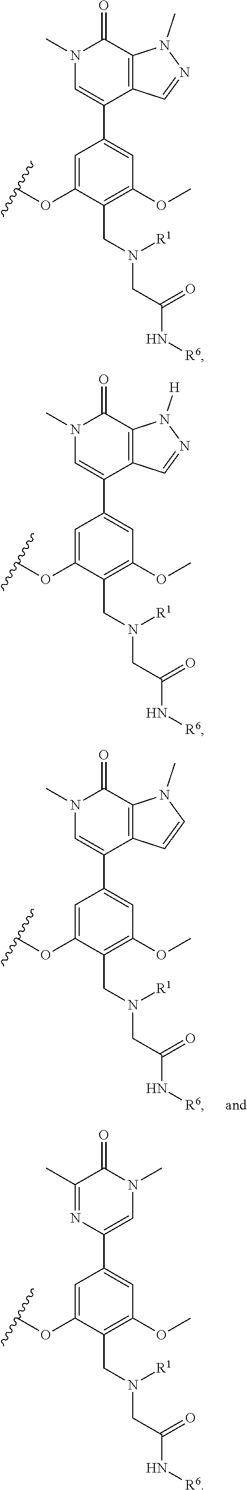

In one embodiment of Formula V, the MTH1-binding moiety

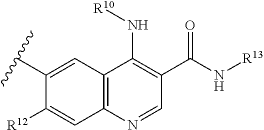

is selected from:

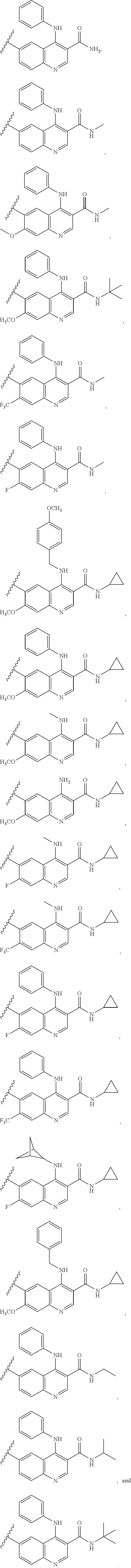

In one embodiment of Formula VI, the MTH1-binding moiety

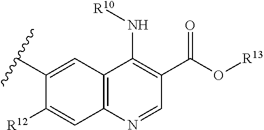

is selected from:

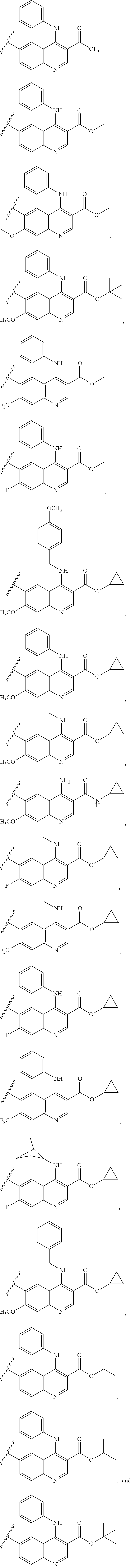

In one embodiment of any one of Formula V or Formula VI, -L²-L³-is selected from:

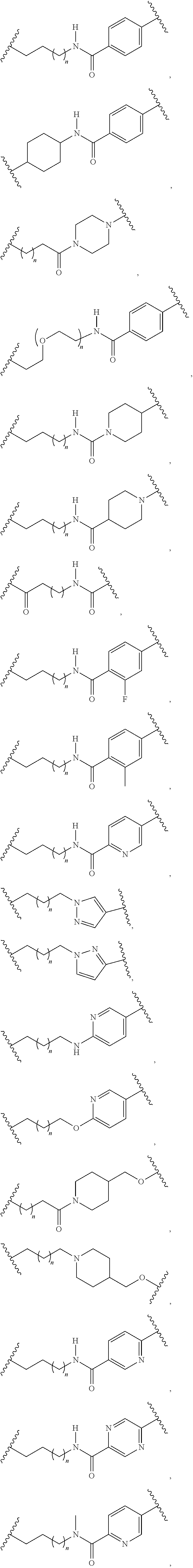

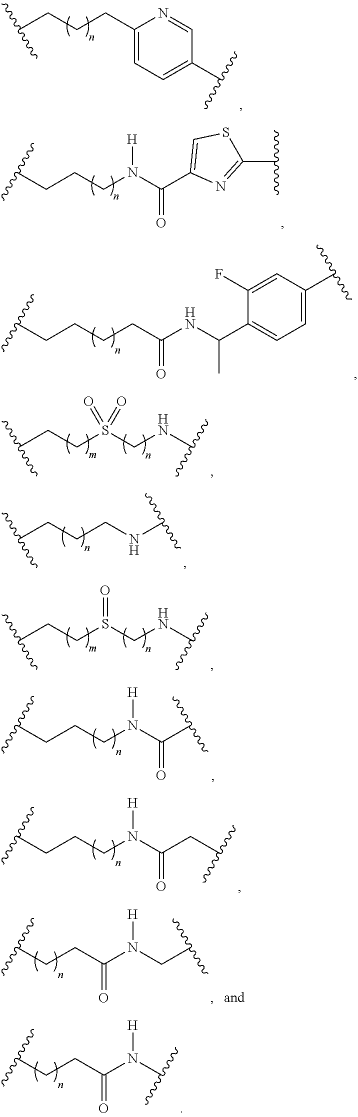

In certain embodiments, the Degron is selected from:

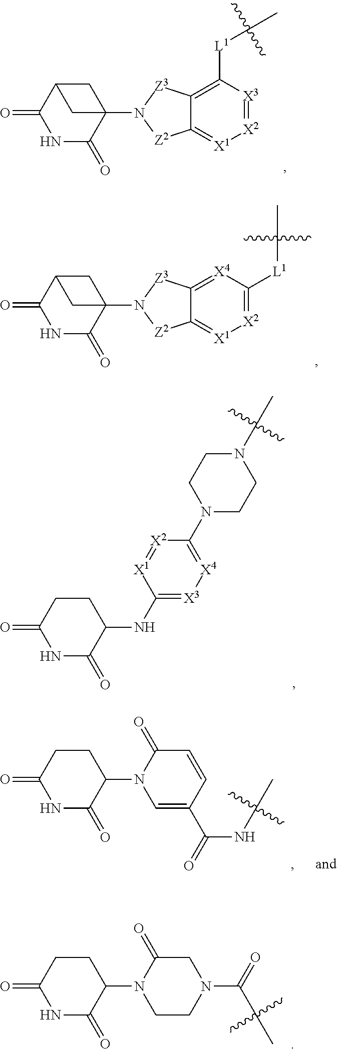

In certain embodiments, the Degron is selected from:

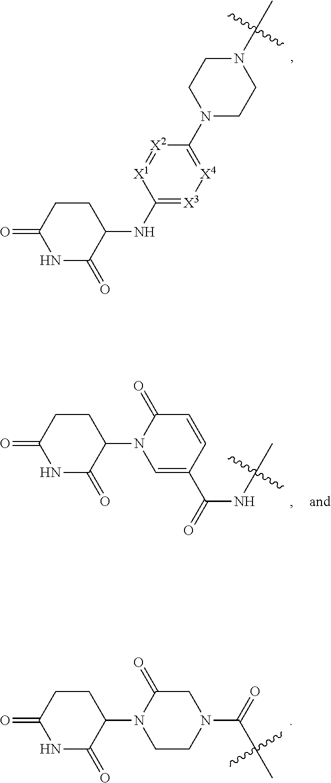

In certain embodiments, the Degron is selected from:

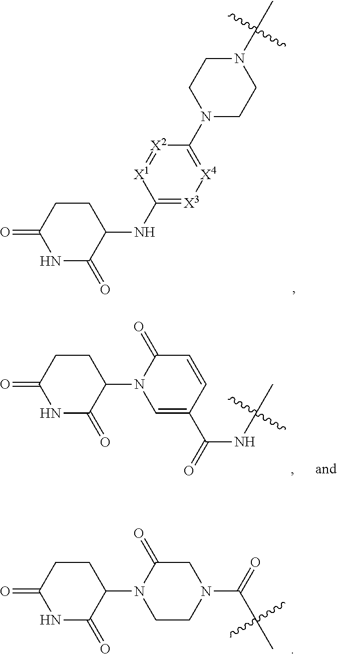

In one embodiment, the Degron is selected from:

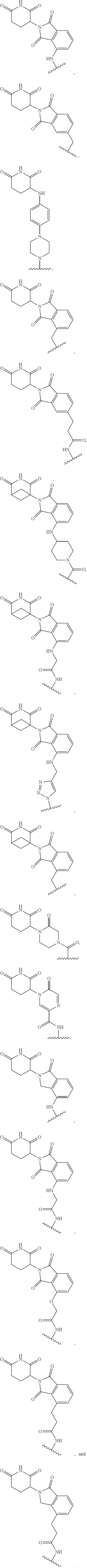

In another embodiment, the Degron is selected from:

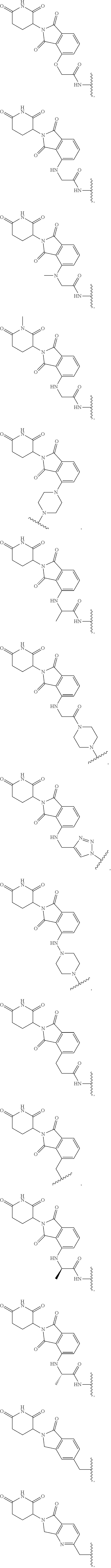

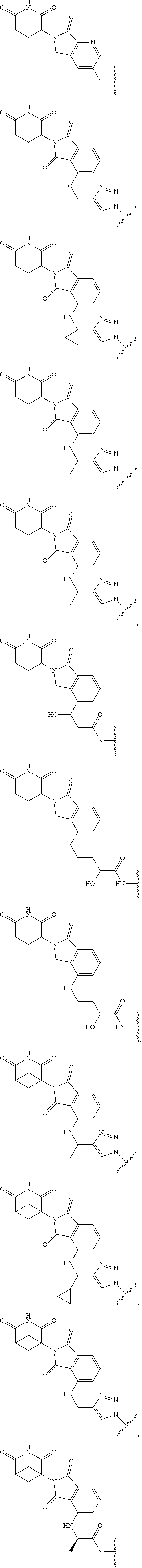

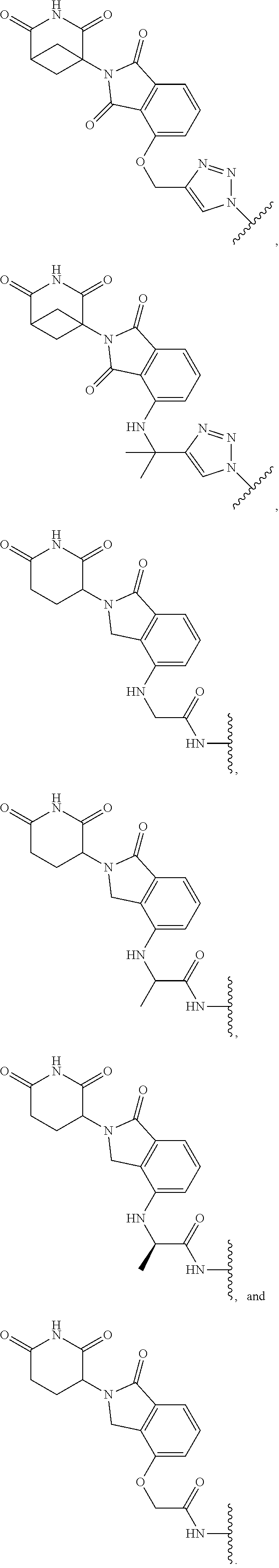 [SMILES: *n1cc(COc2cccc3c2C(=O)N(C24CC(C2)C(=O)NC4=O)C3=O)nn1 ; *n1cc(C(C)(C)Nc2cccc3c2C(=O)N(C24CC(C2)C(=O)NC4=O)C3=O)nn1 ; *NC(=O)CNc1cccc2c1CN(C1CCC(=O)NC1=O)C2=O ; *NC(=O)C(C)Nc1cccc2c1CN(C1CCC(=O)NC1=O)C2=O ; *NC(=O)[C@@H](C)Nc1cccc2c1CN(C1CCC(=O)NC1=O)C2=O ; *NC(=O)COc1cccc2c1CN(C1CCC(=O)NC1=O)C2=O]

In certain embodiments, L¹ is selected from:

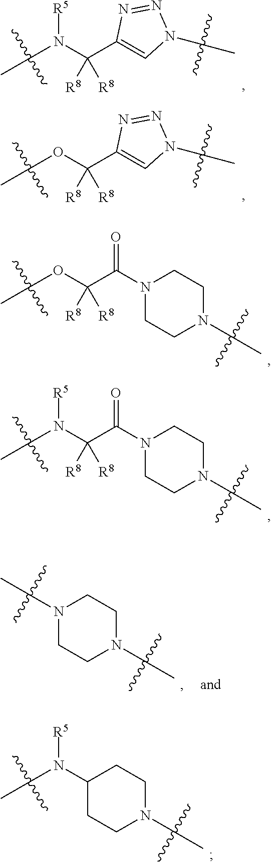

or a pharmaceutically acceptable salt thereof.

In one embodiment, L³ is selected from bond, aryl, heterocycle, and heteroaryl.

In one embodiment, L³ is selected from

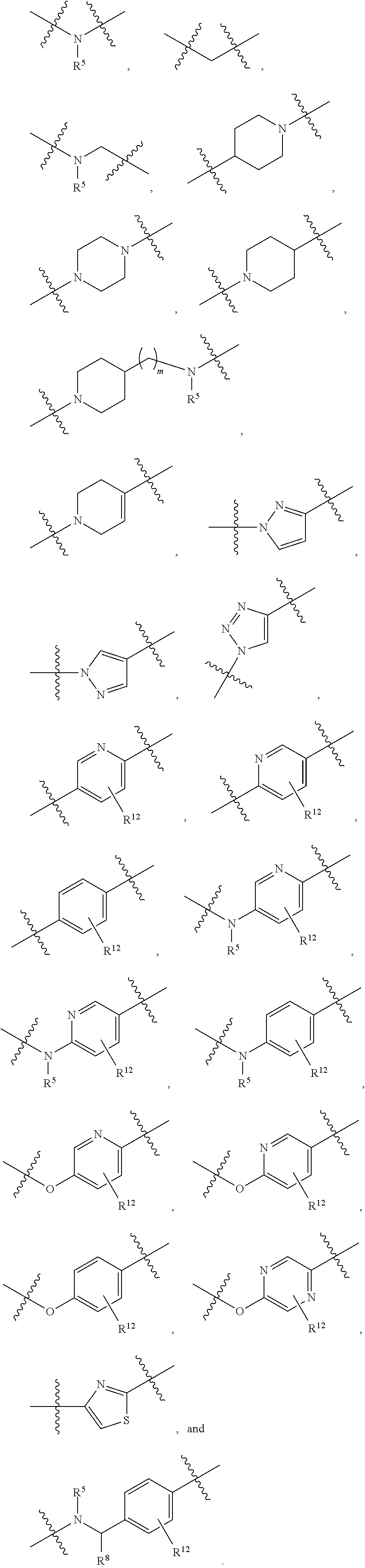

In one embodiment, the compound of Formula V is selected from:

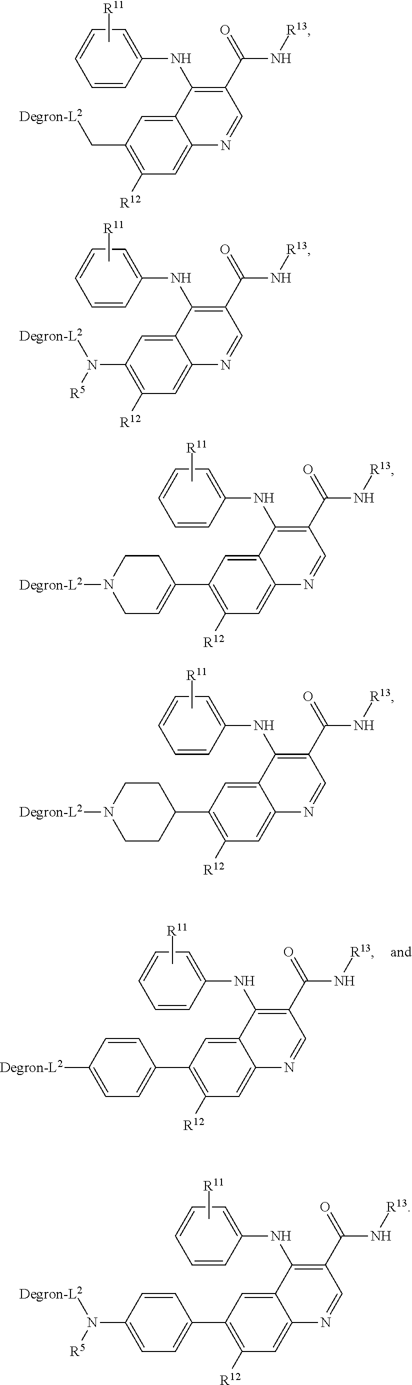

### Non-Limiting Examples of BRD9-Binding Compounds of Formula I

In some embodiments, the compound of Formula I is selected from:

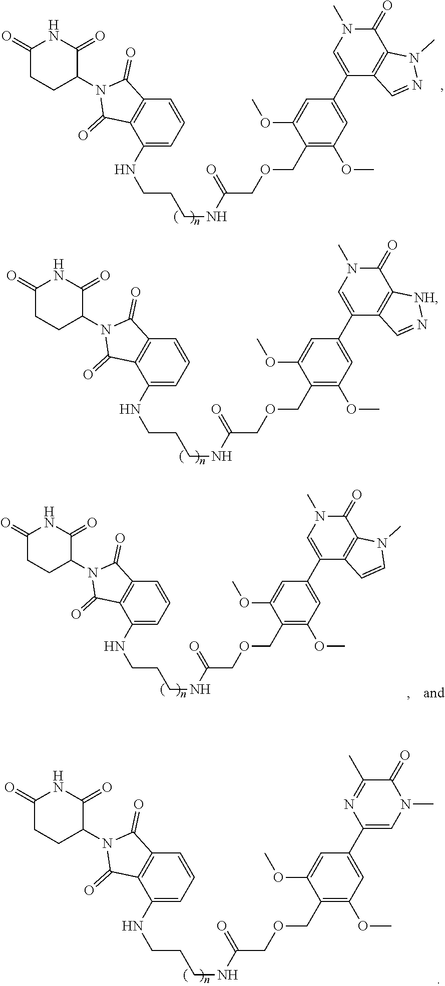

In some embodiments, the compound of Formula I is selected from:

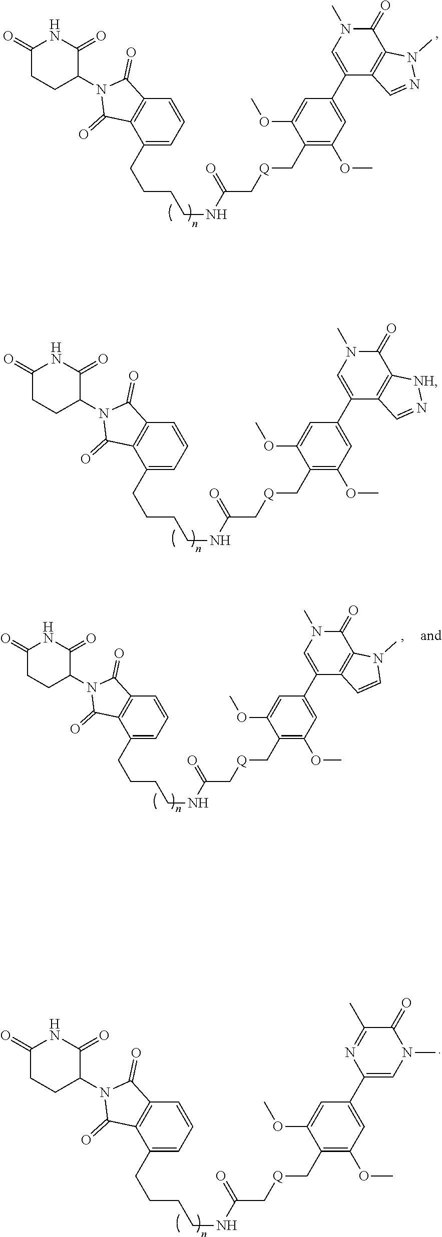

In some embodiments, the compound of Formula I is selected from:

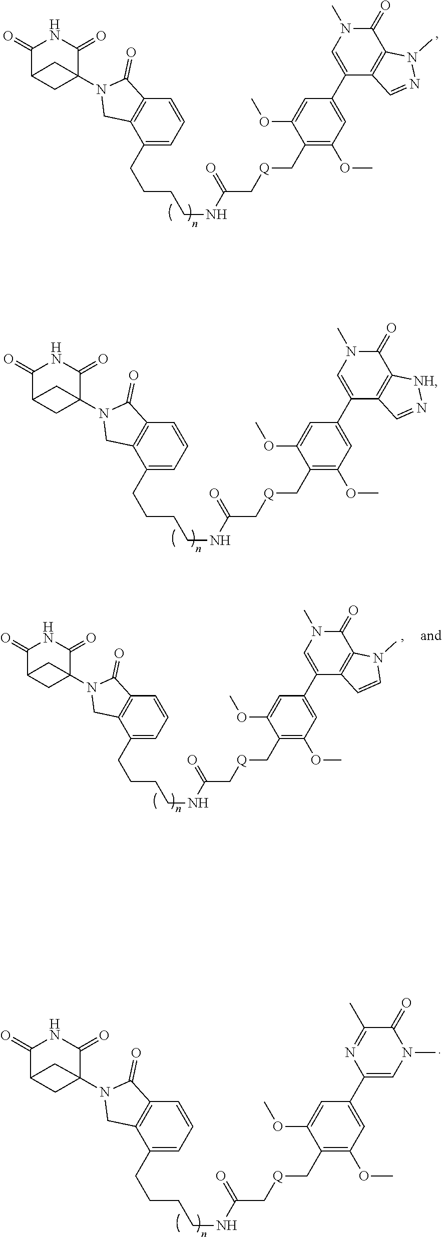

In some embodiments, the compound of Formula I is selected from:

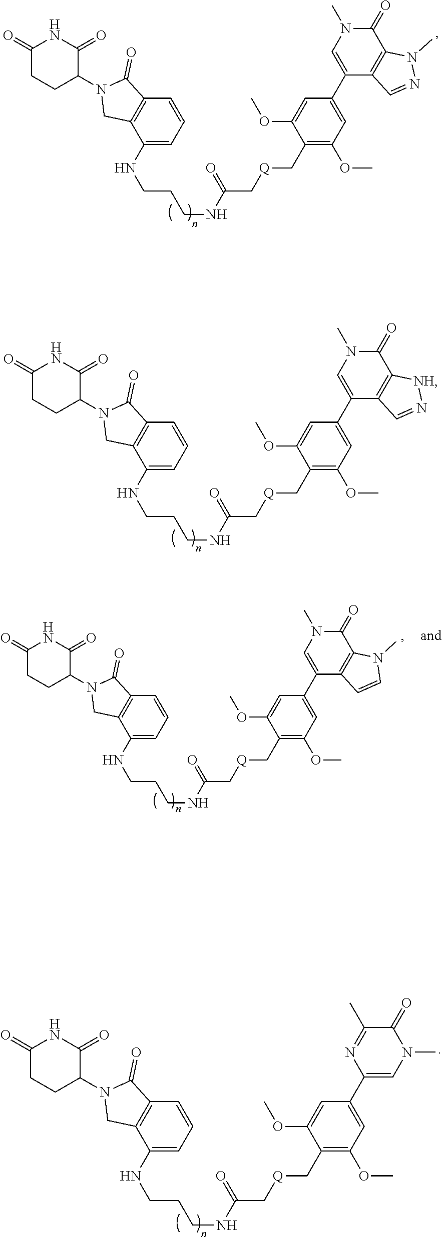

In some embodiments, the compound of Formula I is selected from:

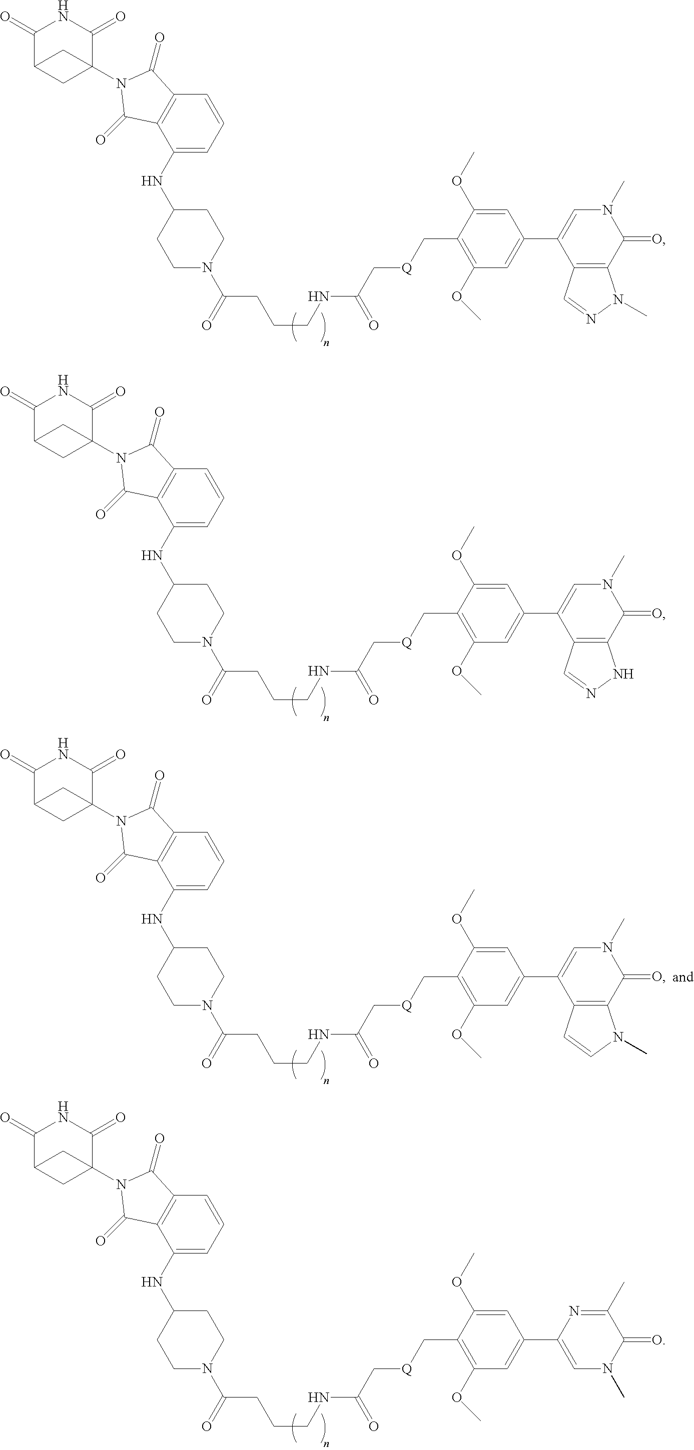

In some embodiments, the compound of Formula I is selected from:

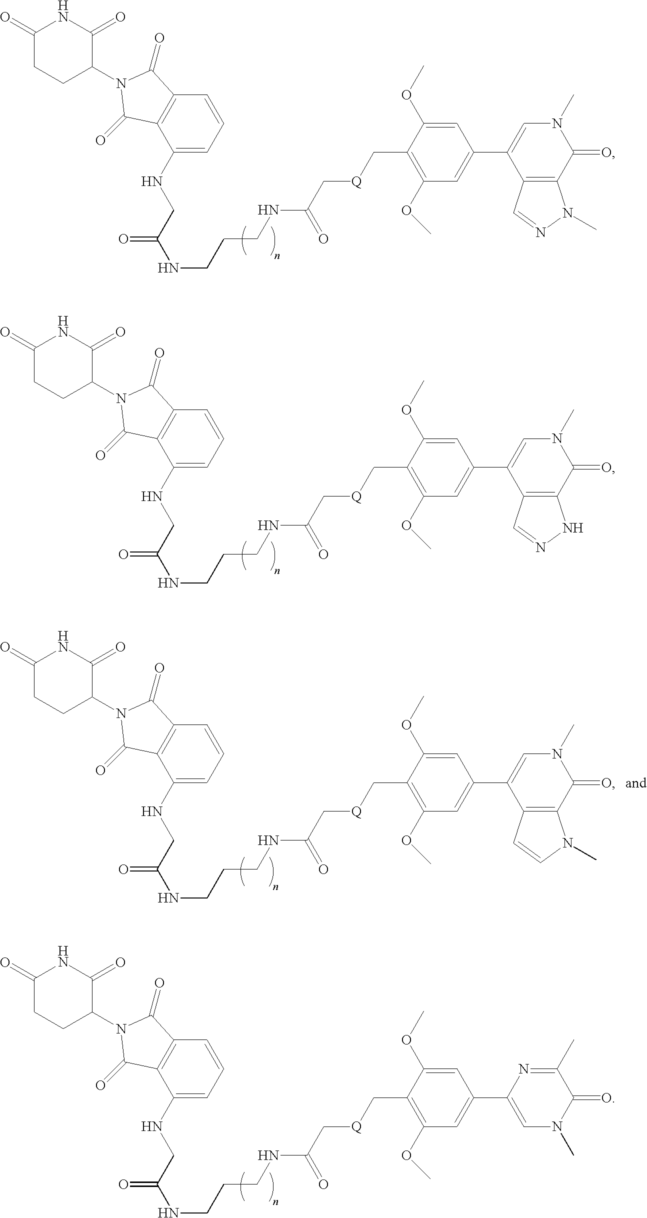

In some embodiments, the compound of Formula I is selected from:

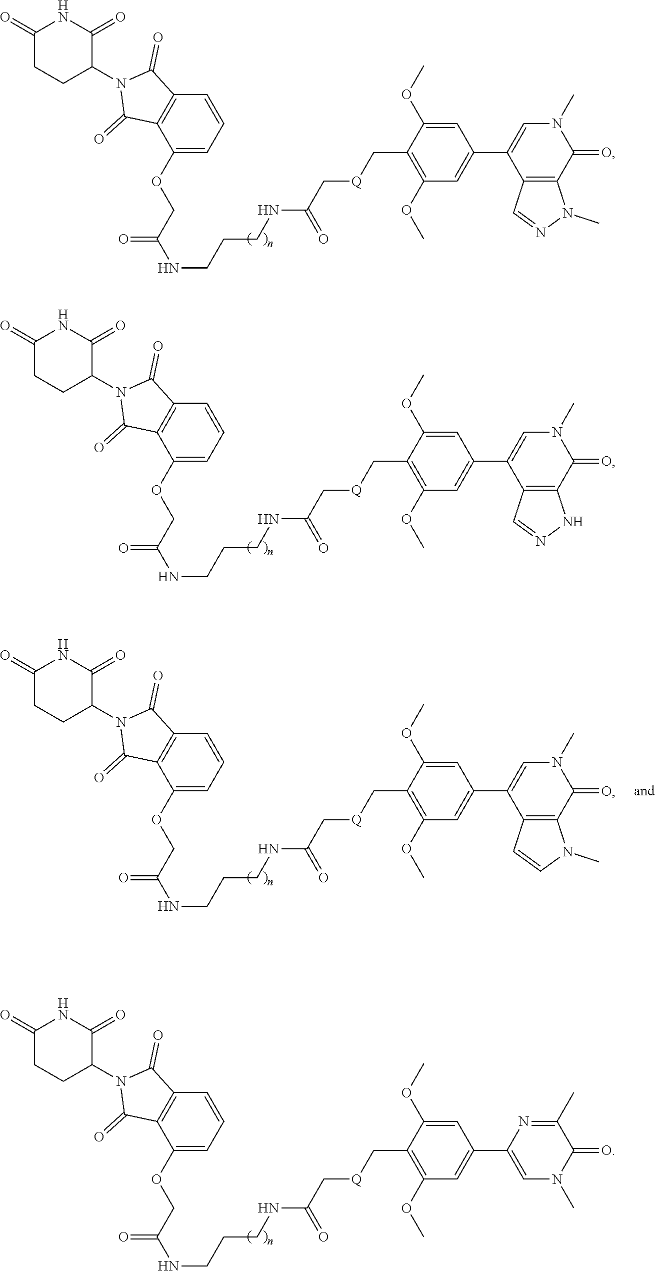

In some embodiments, the compound of Formula I is selected from:

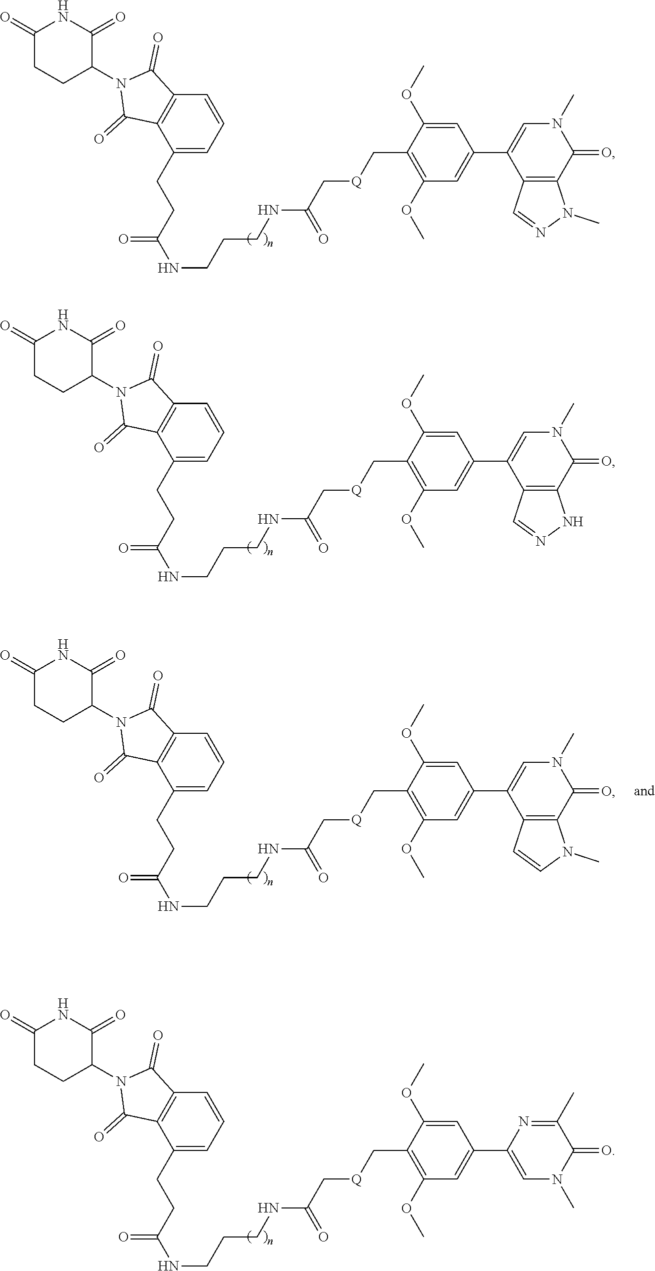

In some embodiments, the compound of Formula I is selected from:

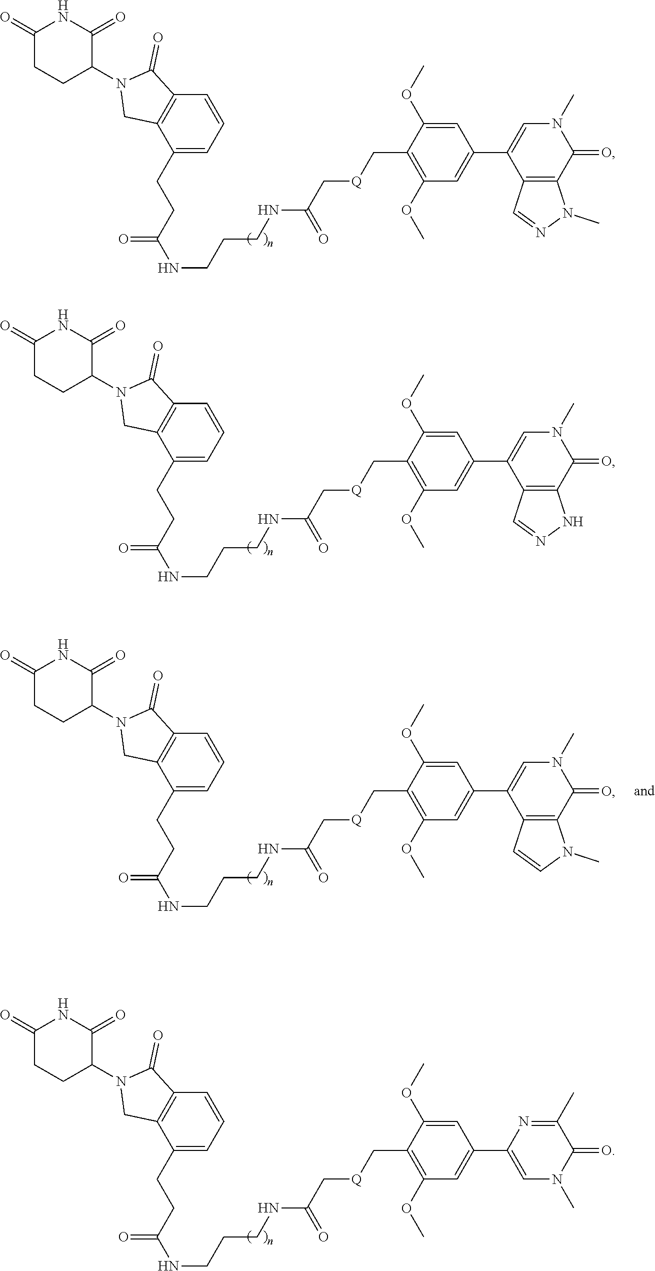

In some embodiments, the compound of Formula I is selected from:

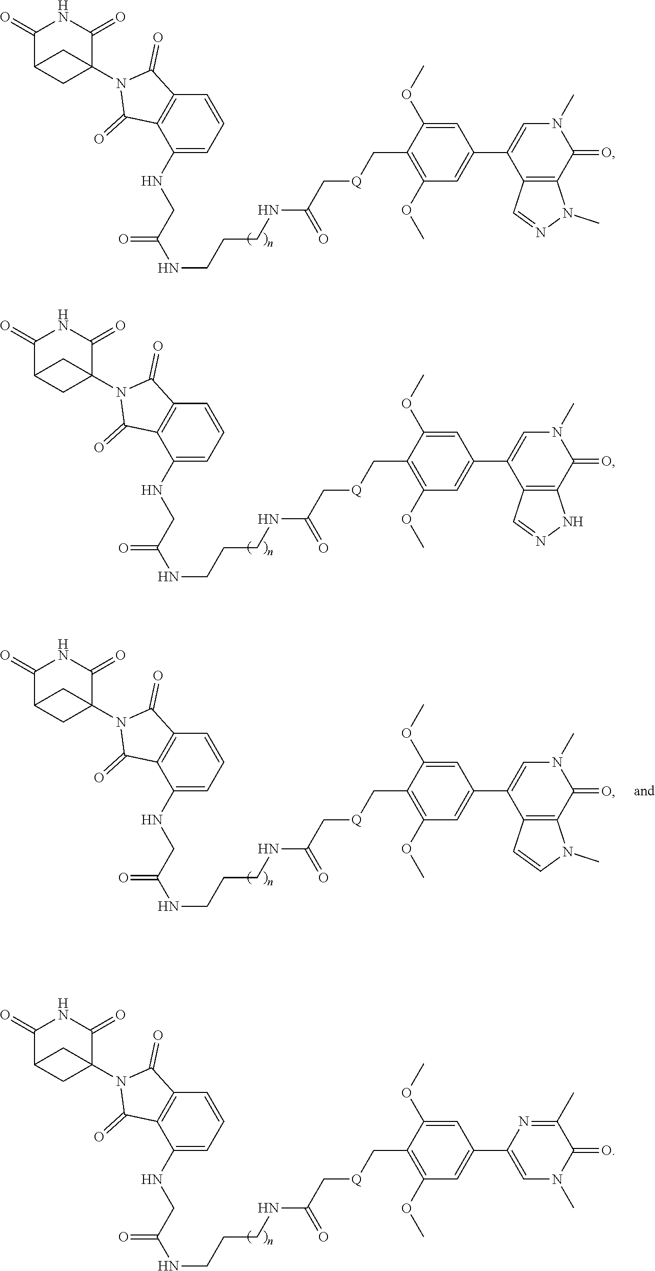

In some embodiment, the compound of Formula I is selected from:

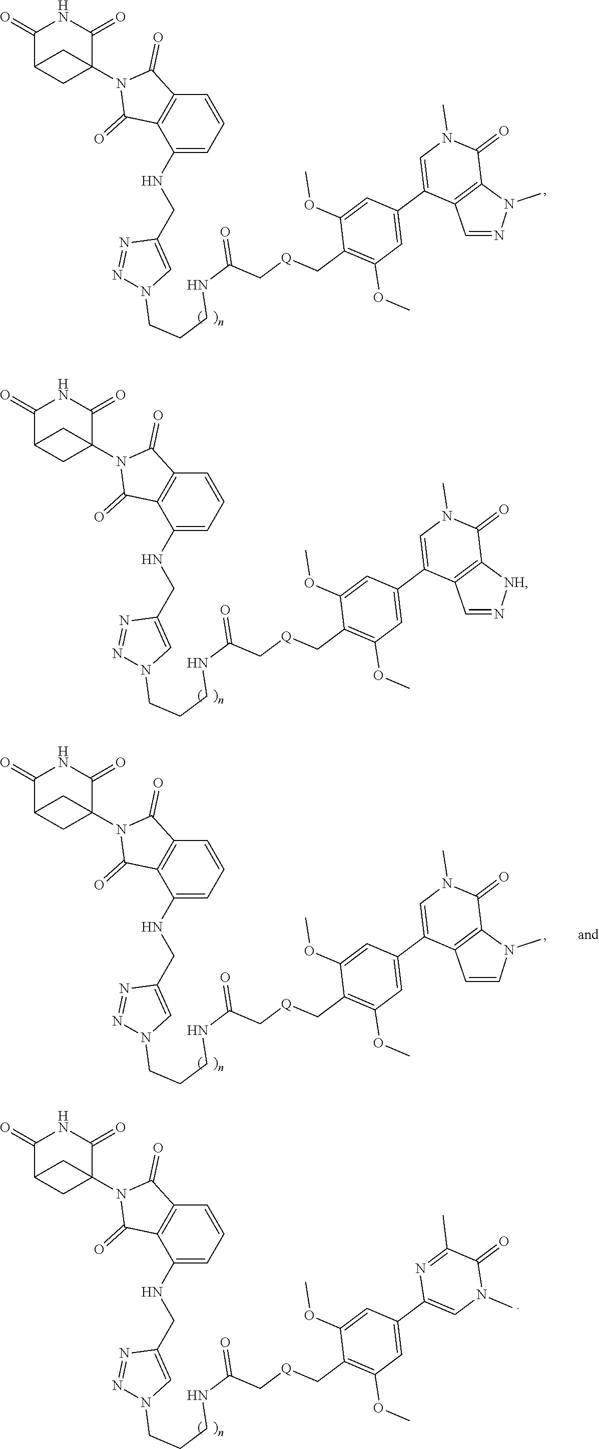

In some embodiments, the compound of Formula I is selected from:

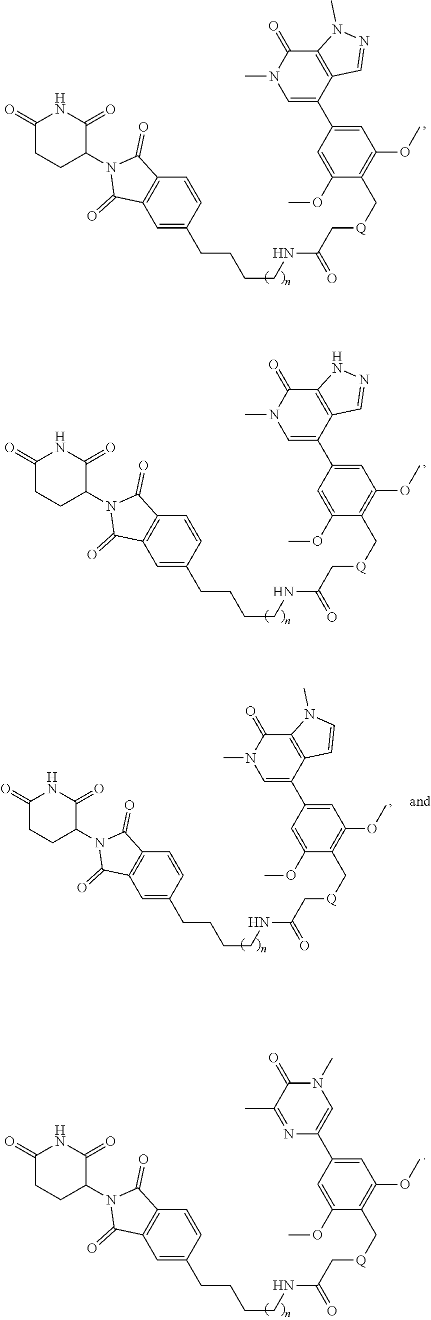

In some embodiments, the compound of Formula I is selected from:

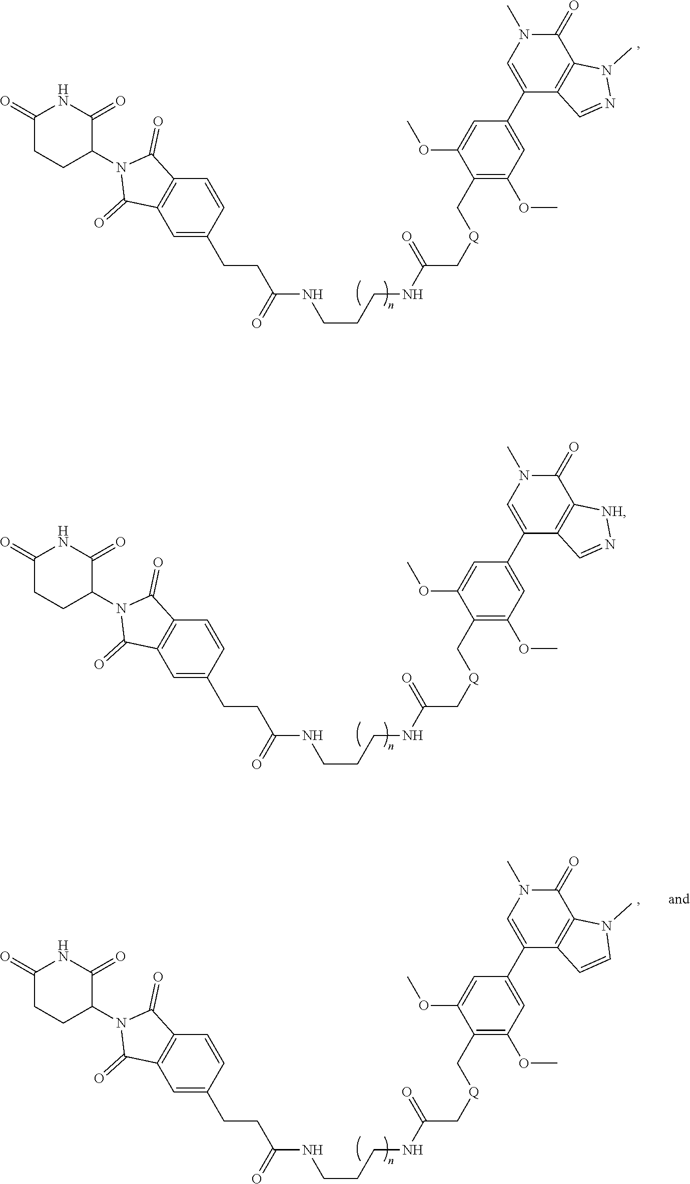

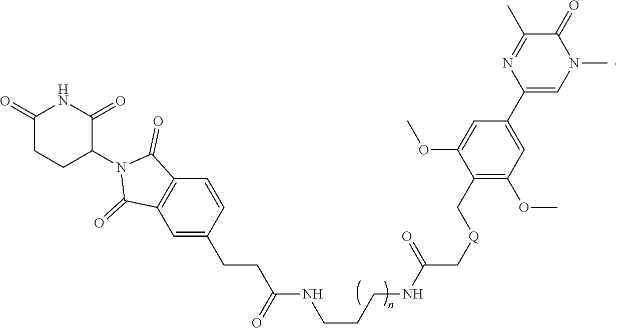

In some embodiments, the compound of Formula I is selected from:

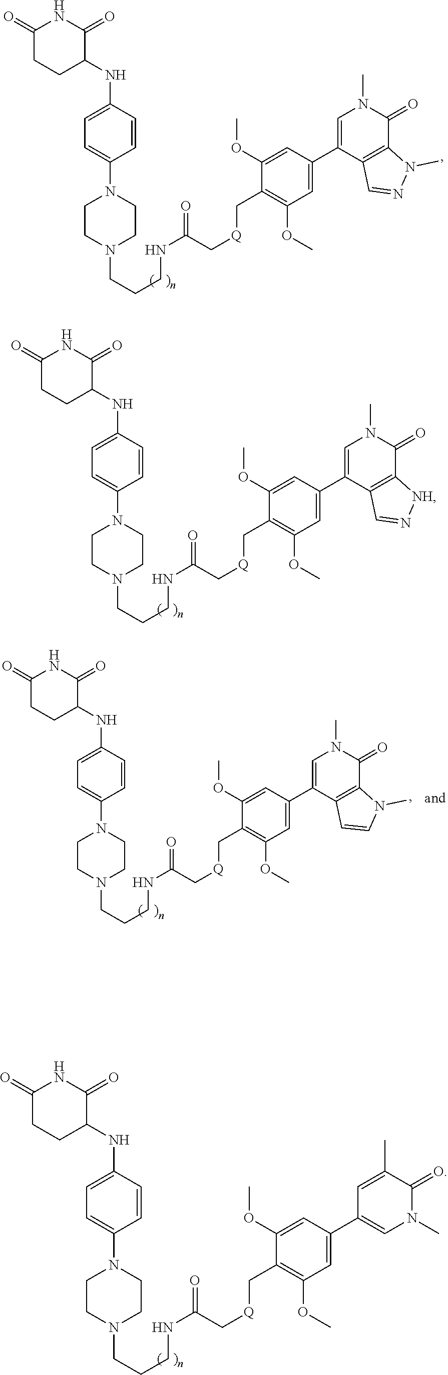

In some embodiments, the compound of Formula I is selected from:

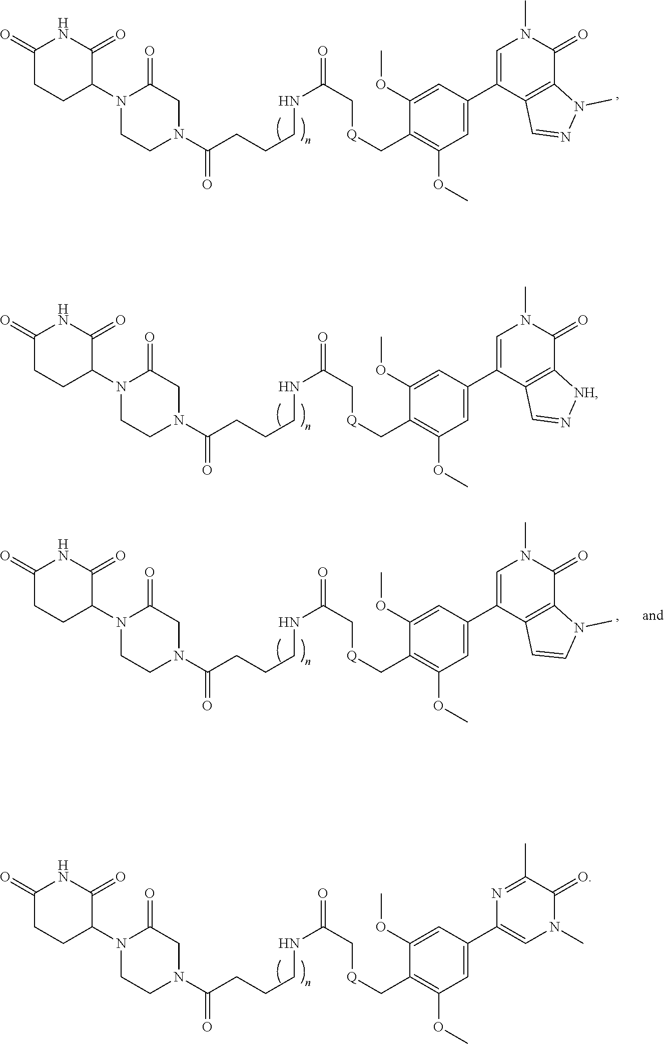

In some embodiments, the compound of Formula I is selected from:

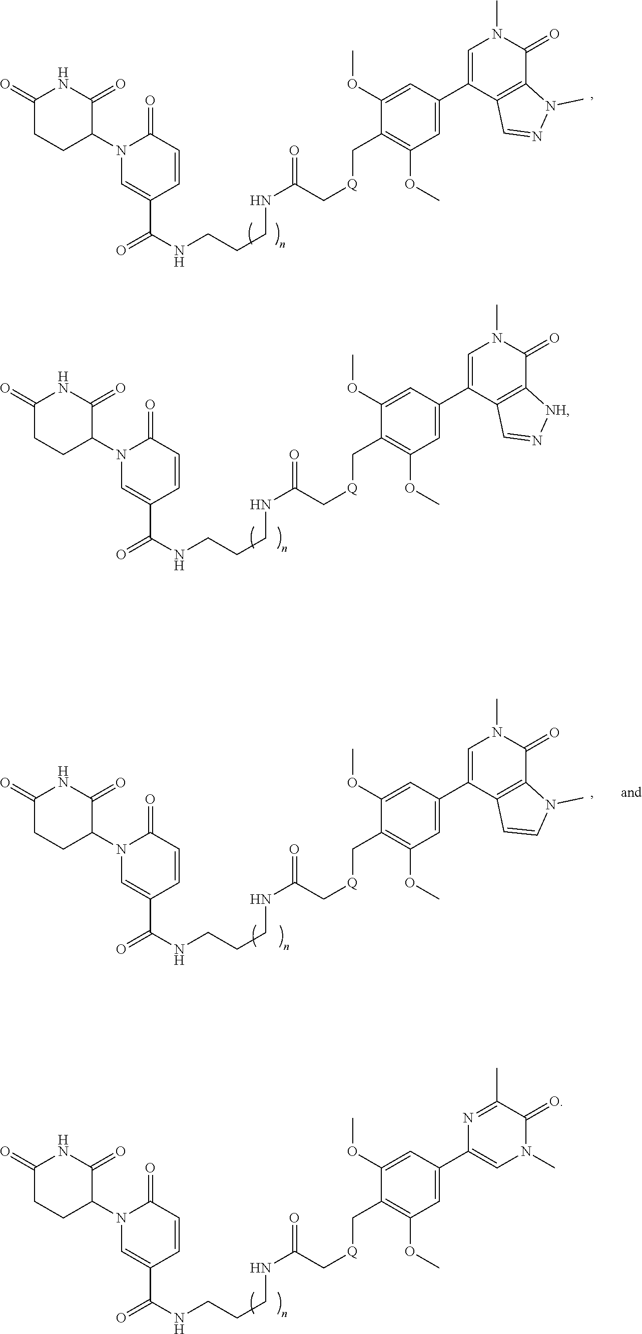

In some embodiments of Formula I,

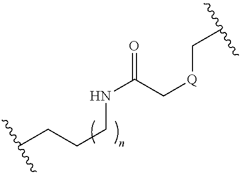

is selected from:

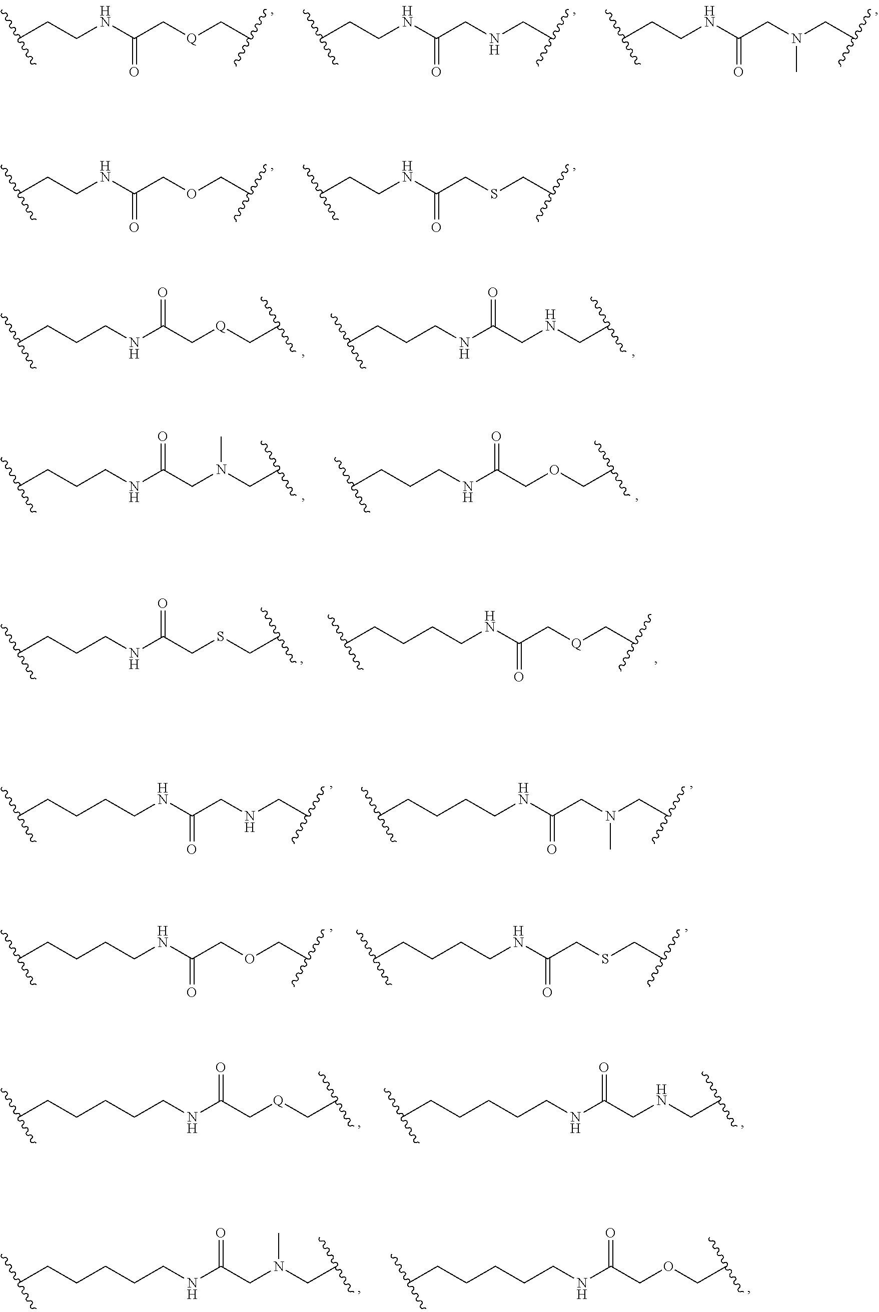

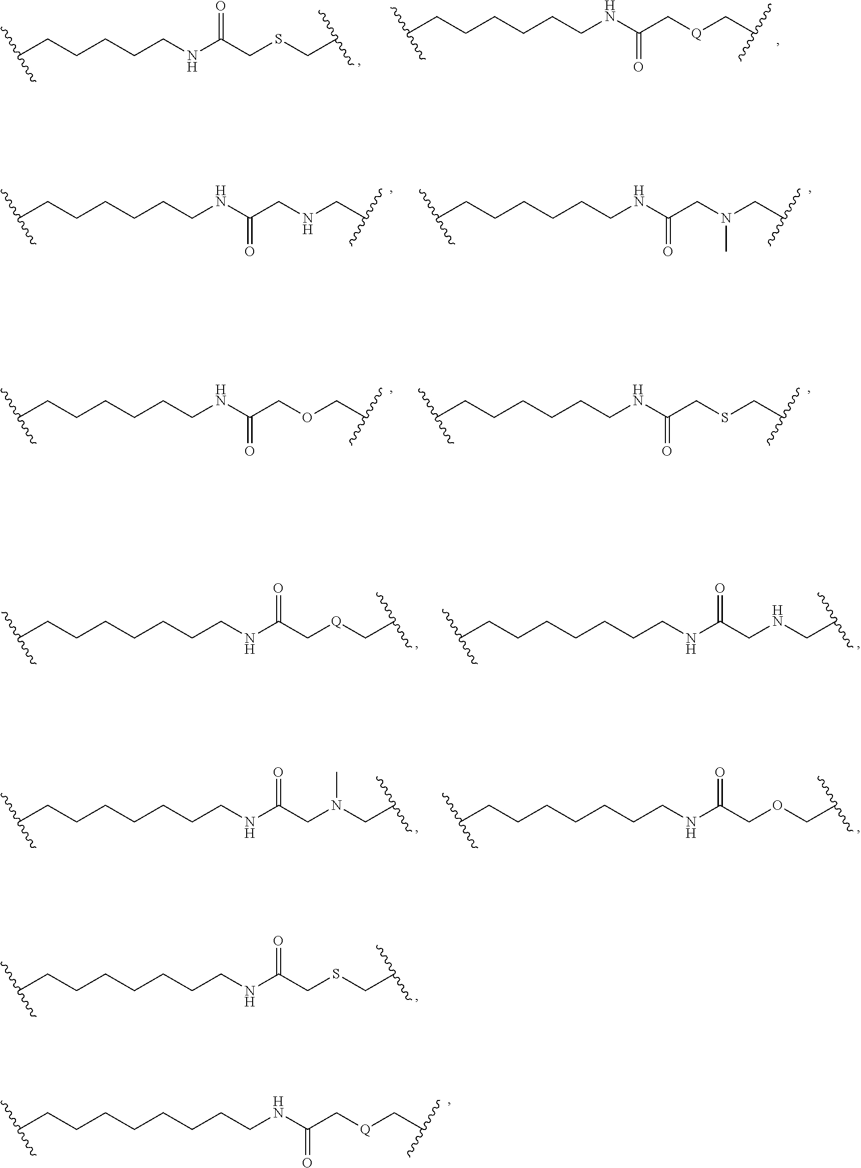

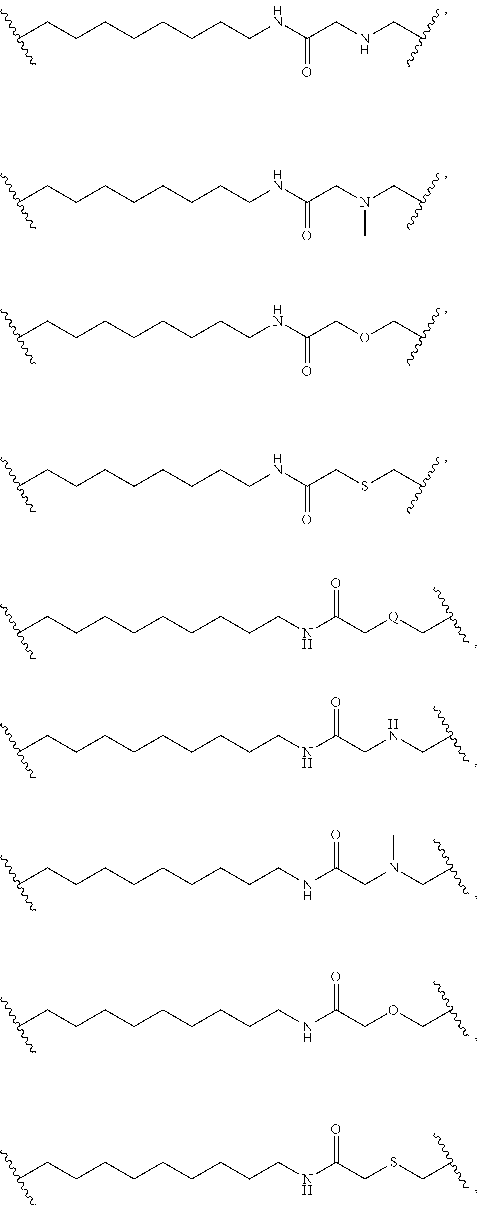

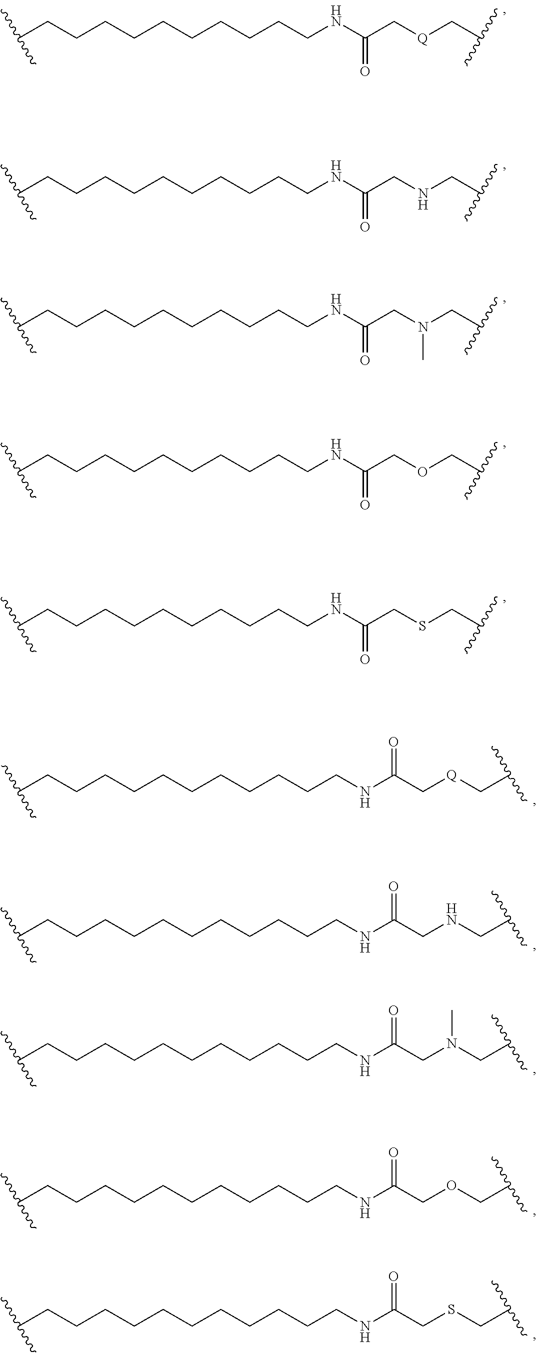

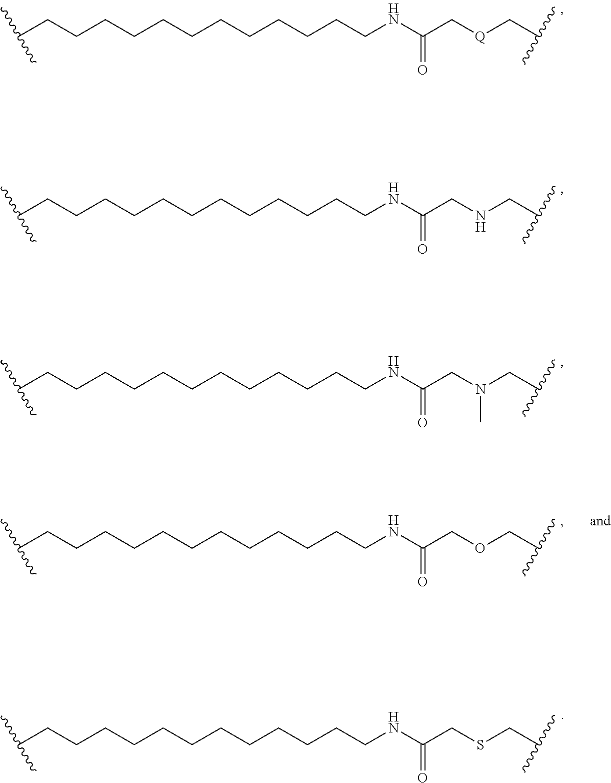

### Non-Limiting Examples of BRD9-Binding Compounds of Formula II

In some embodiments, the compound of Formula II is selected from:

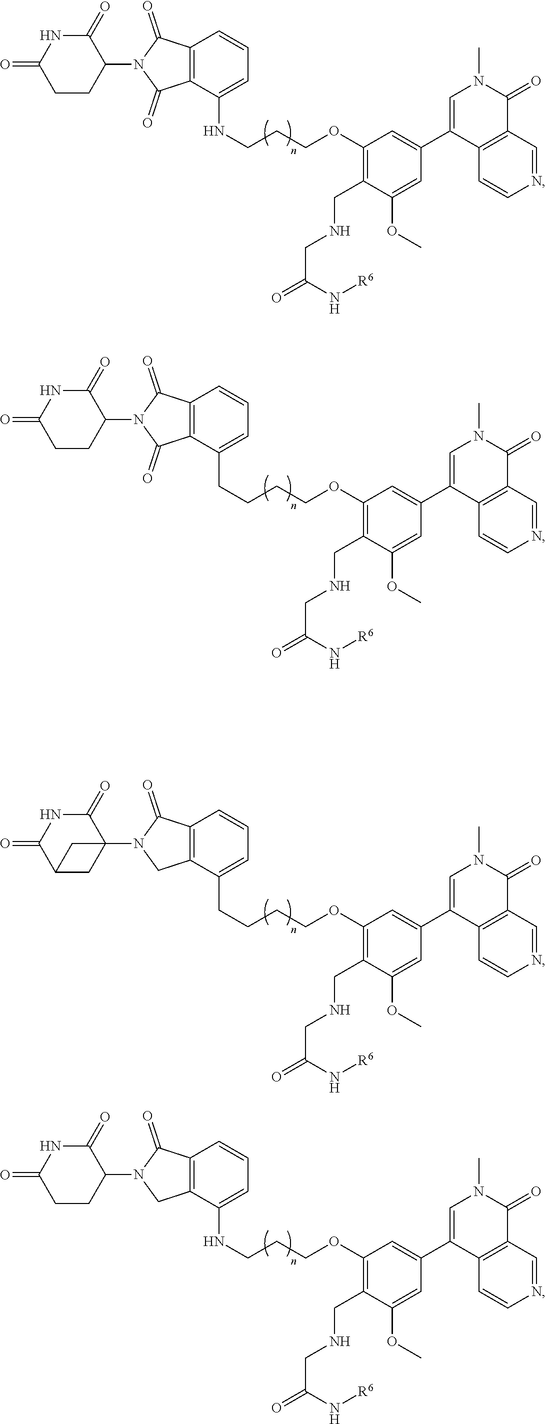

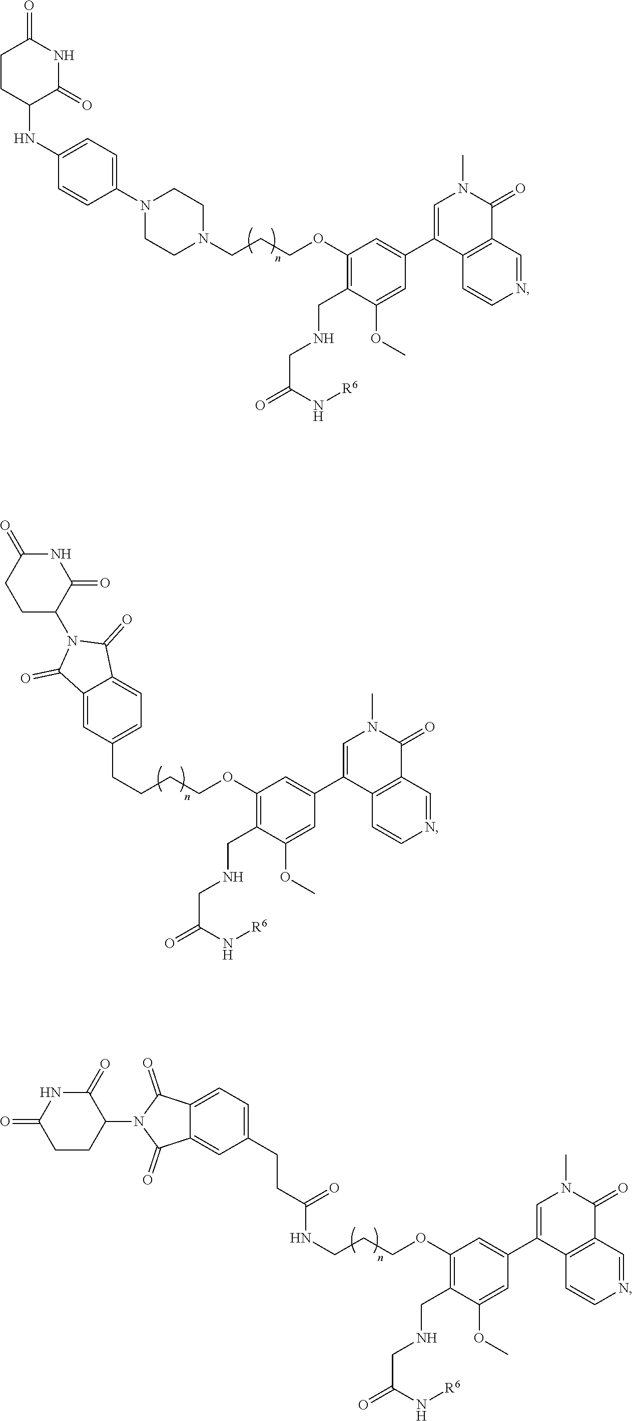

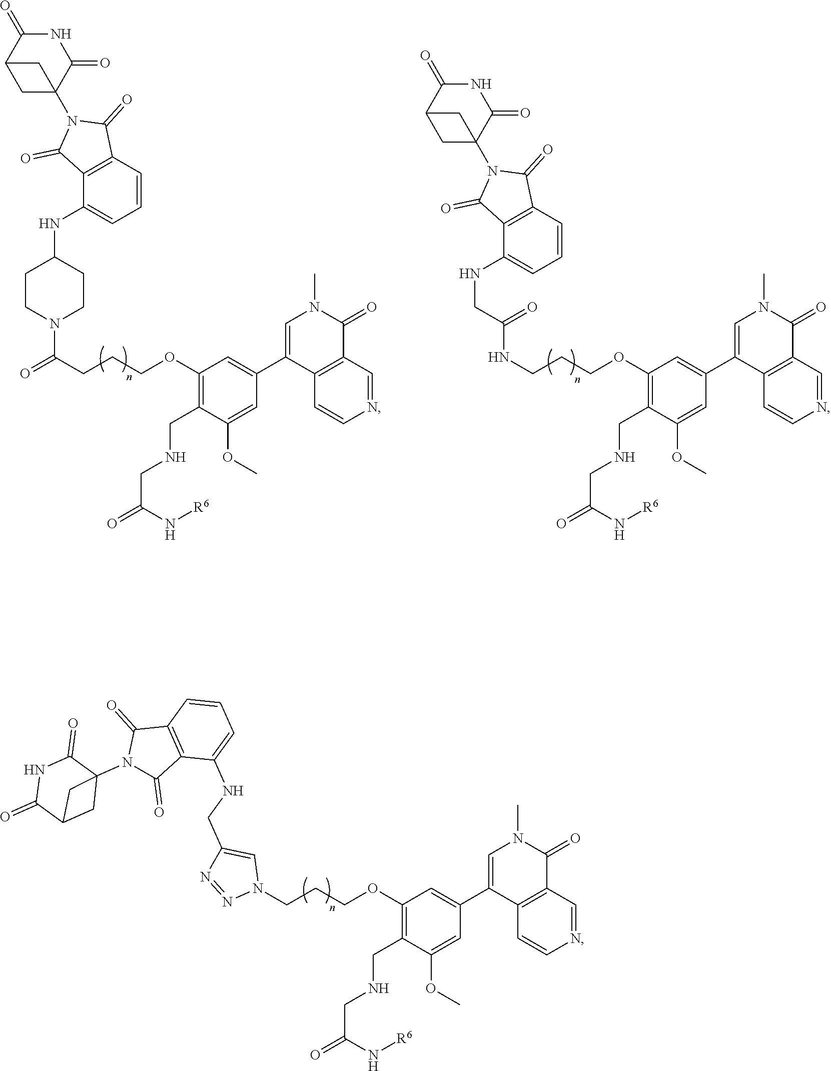

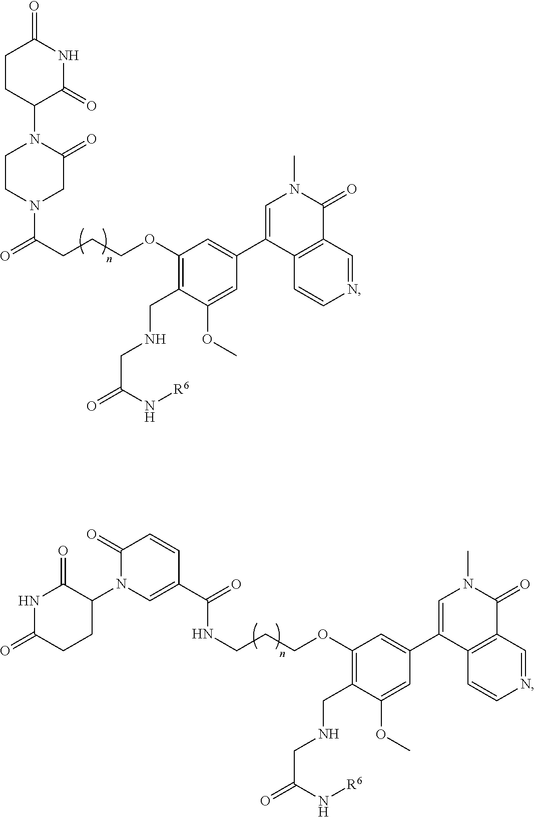

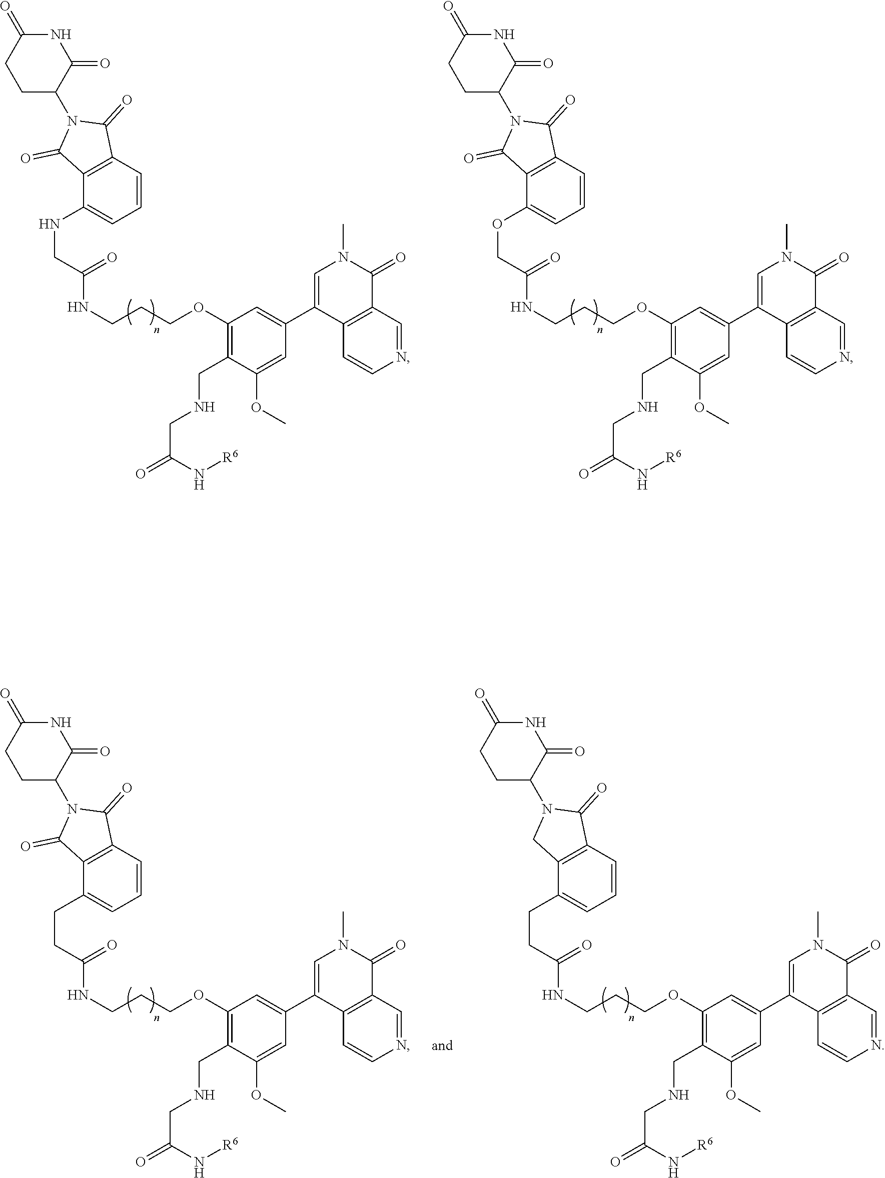

In some embodiments, the compound of Formula II is selected from:

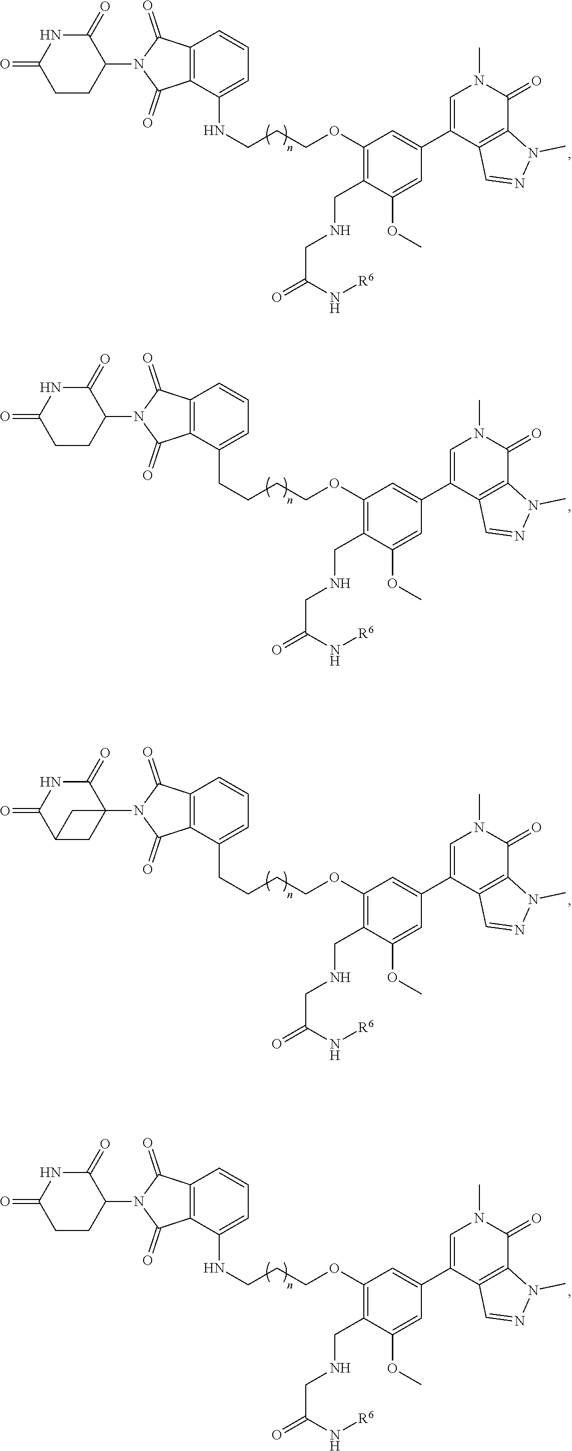

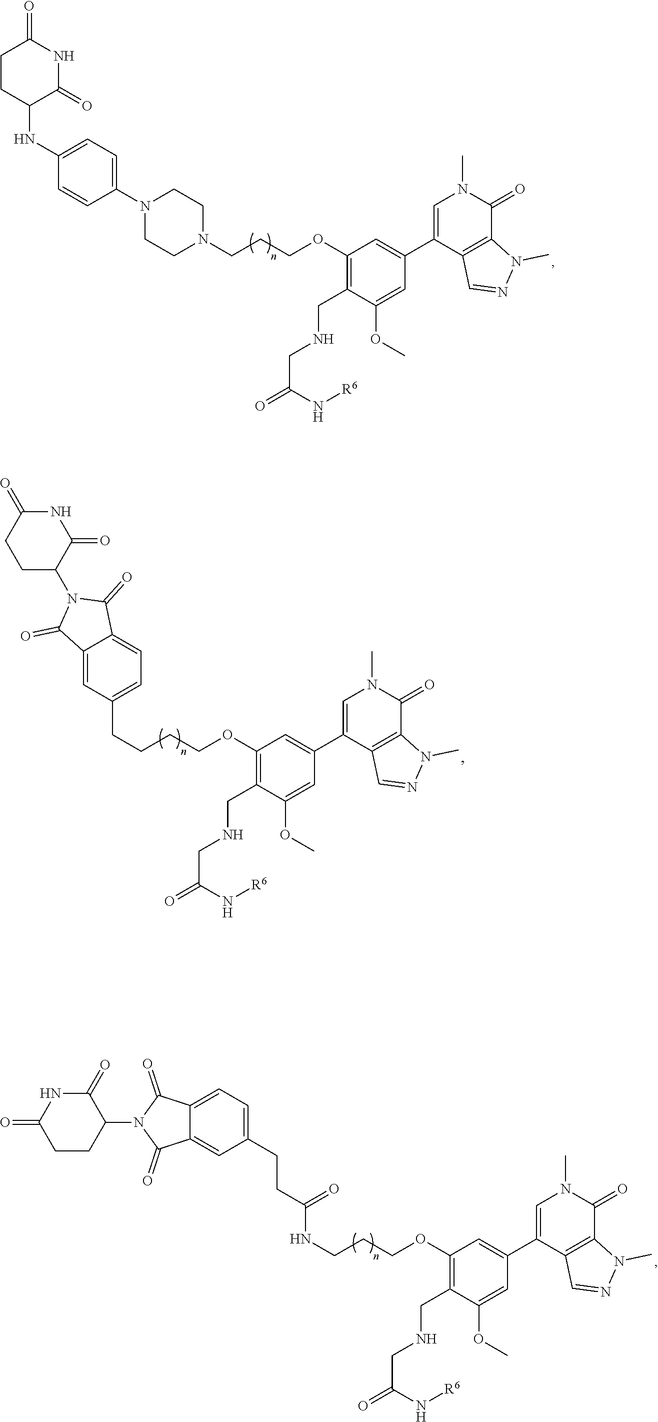

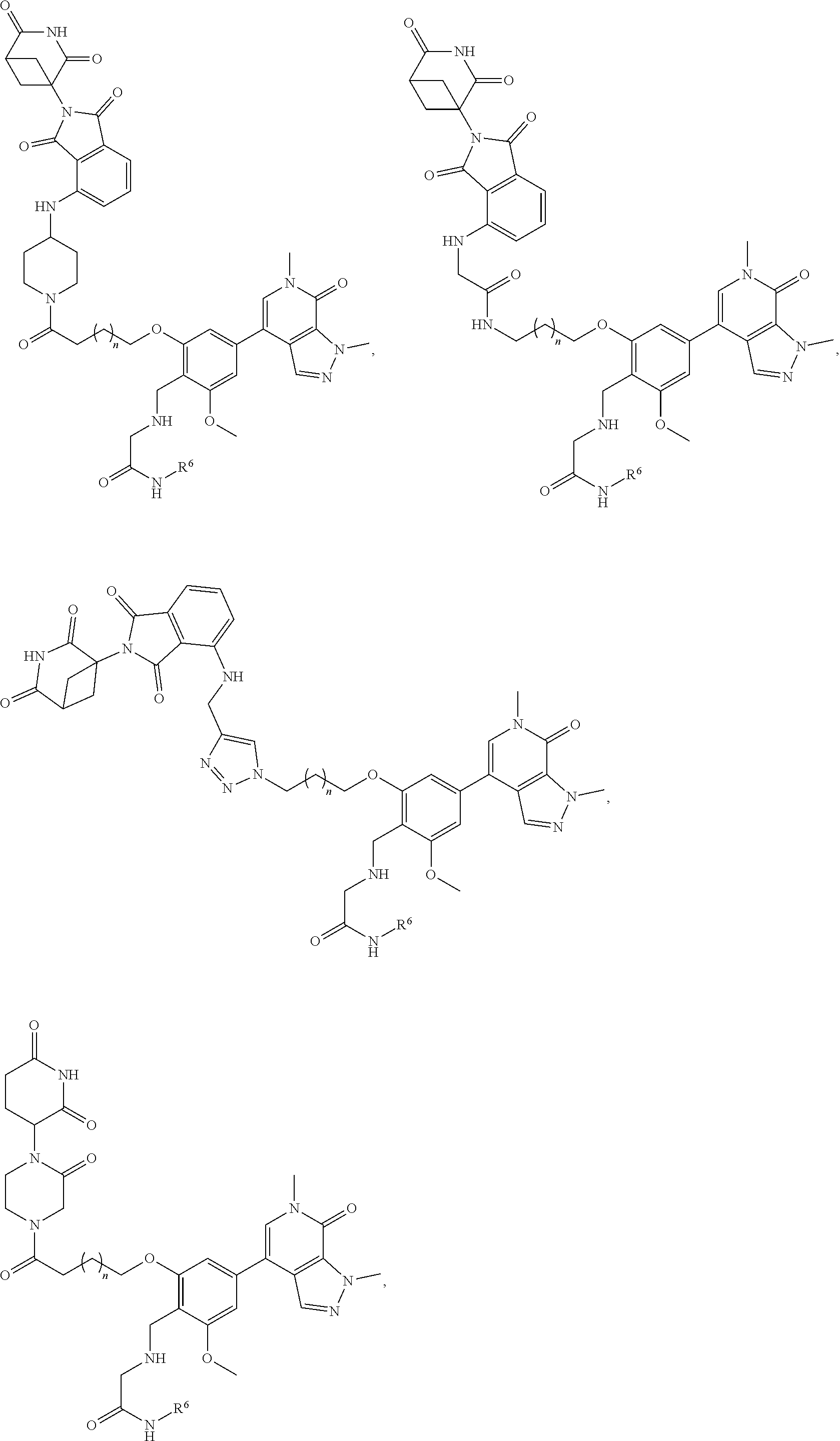

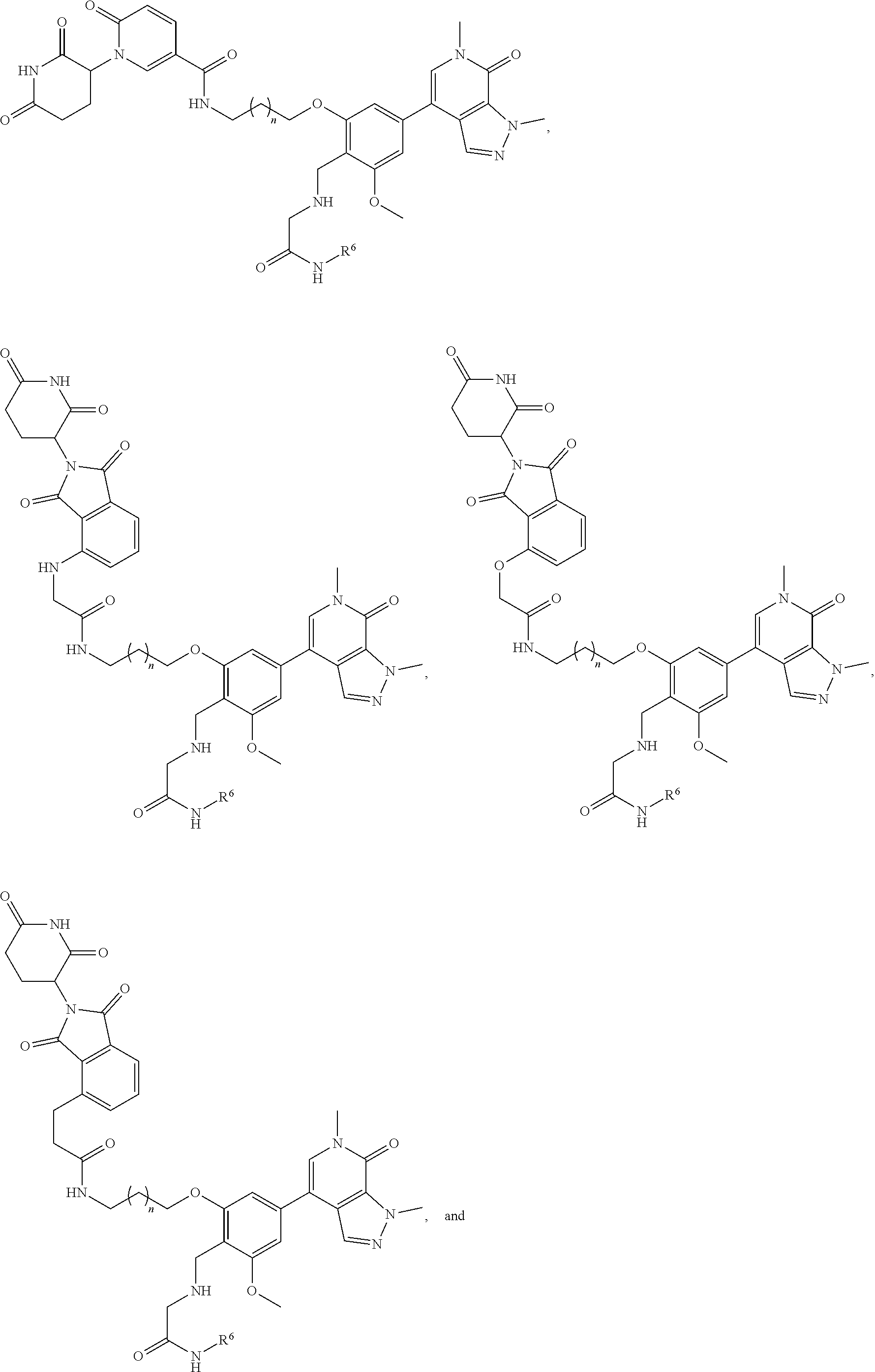

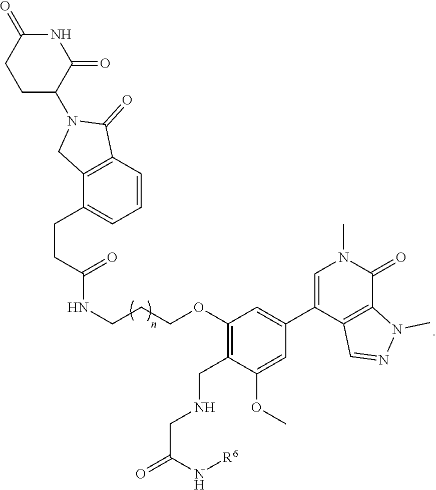

In some embodiments, the compound of Formula II is selected from:

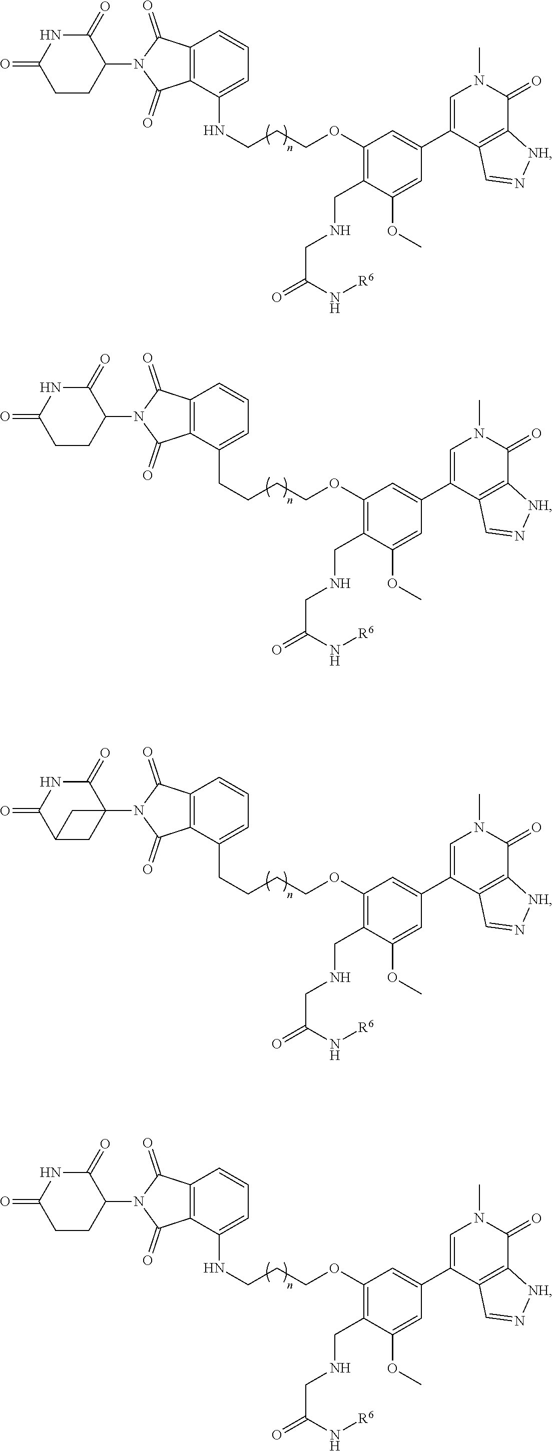

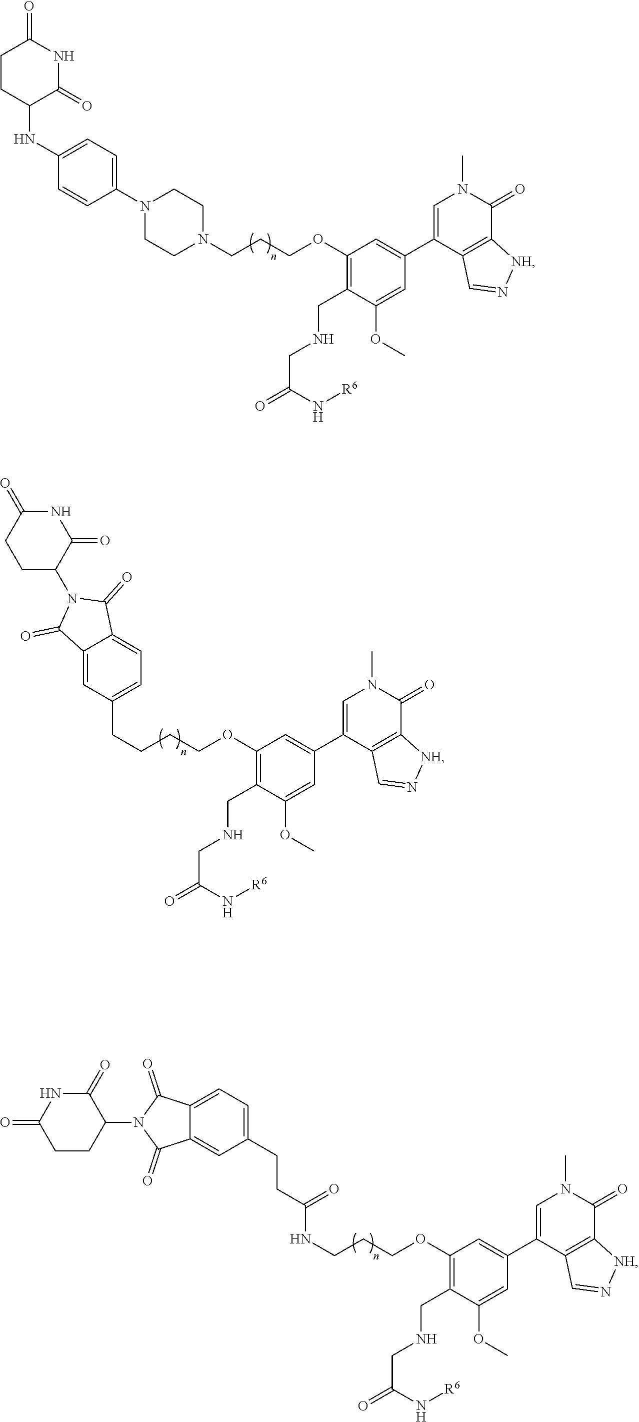

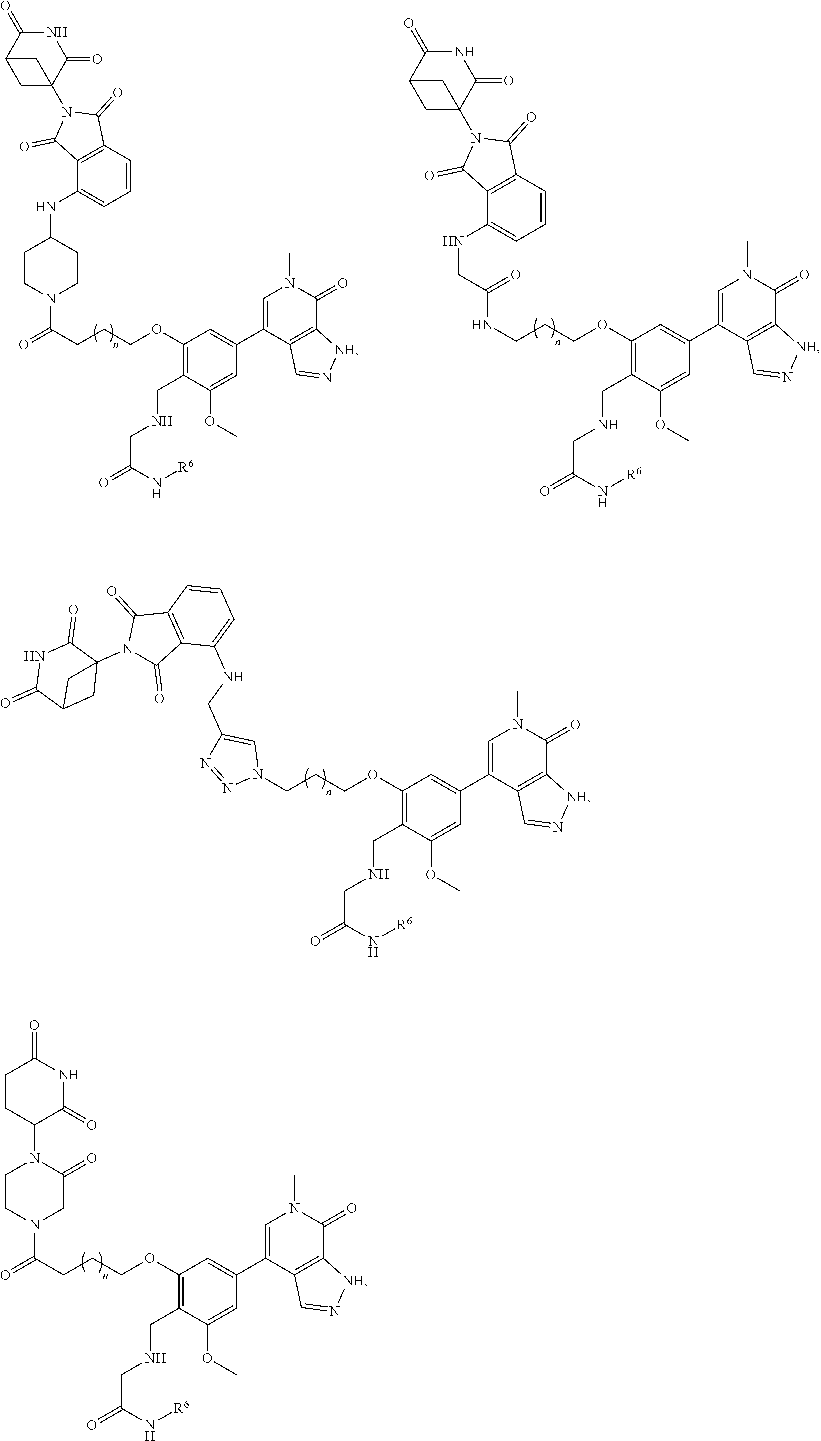

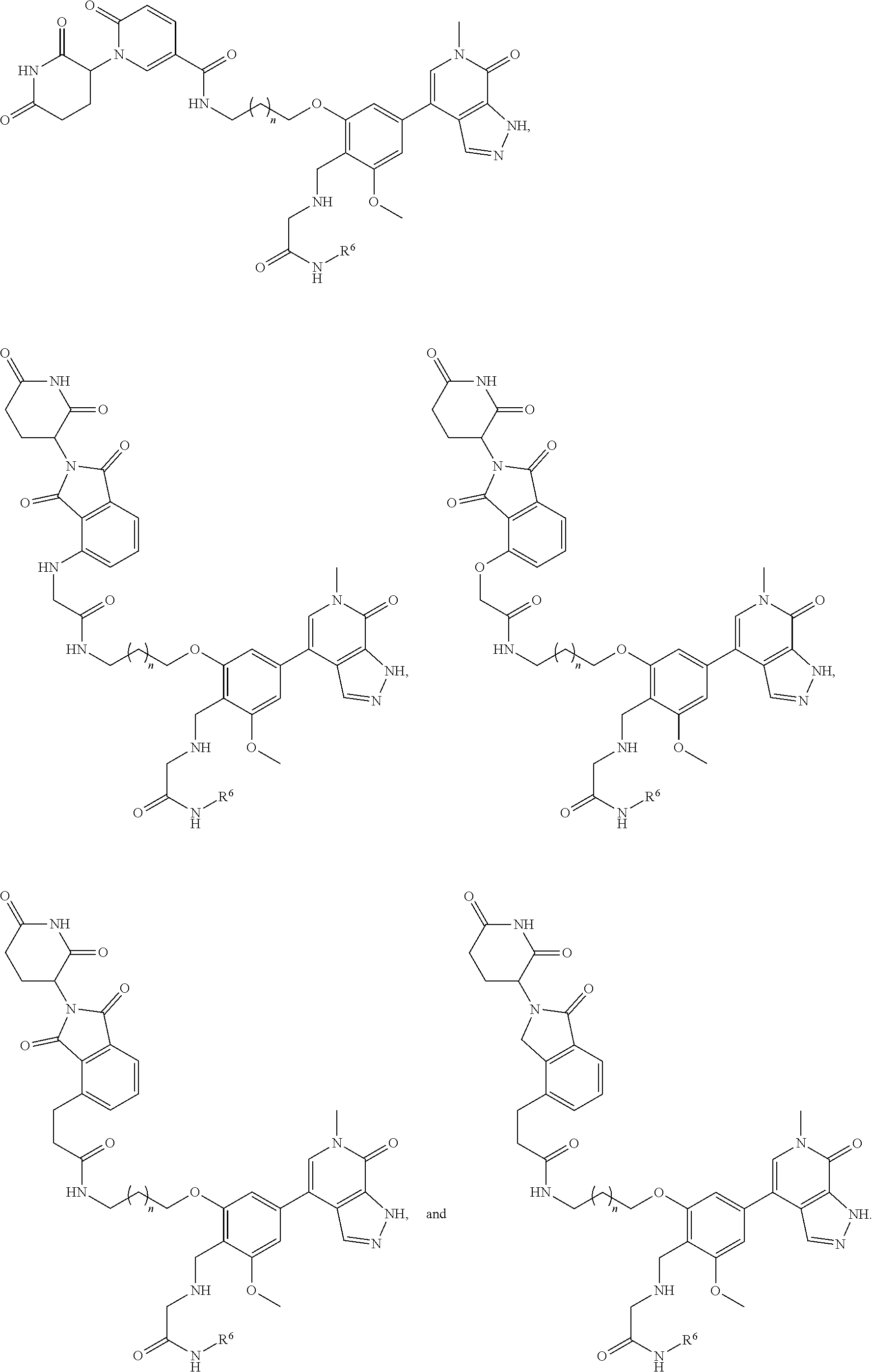

In some embodiments, the compound of Formula II is selected from:

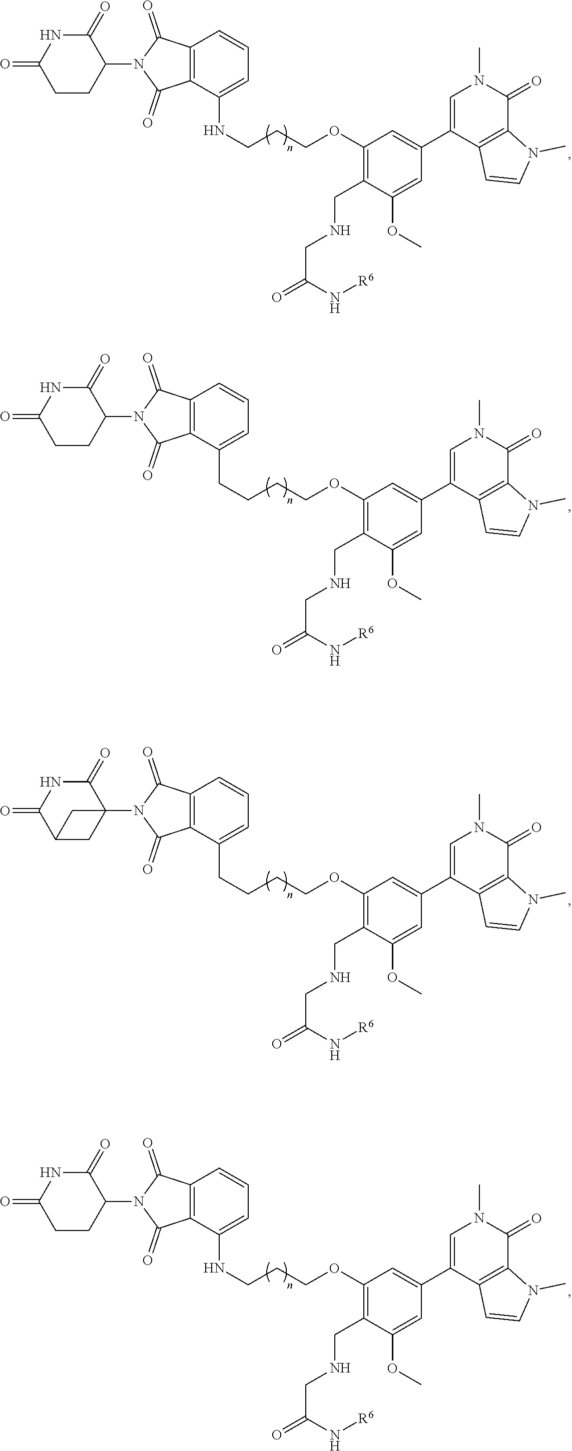

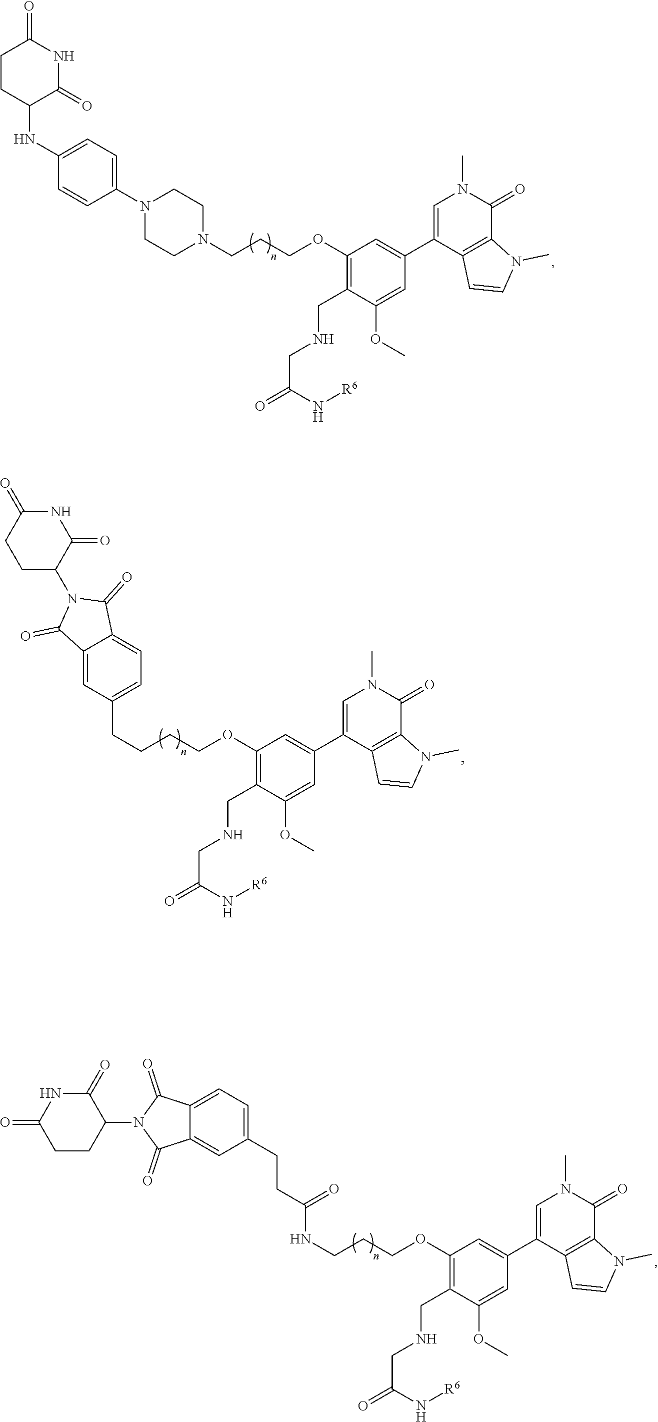

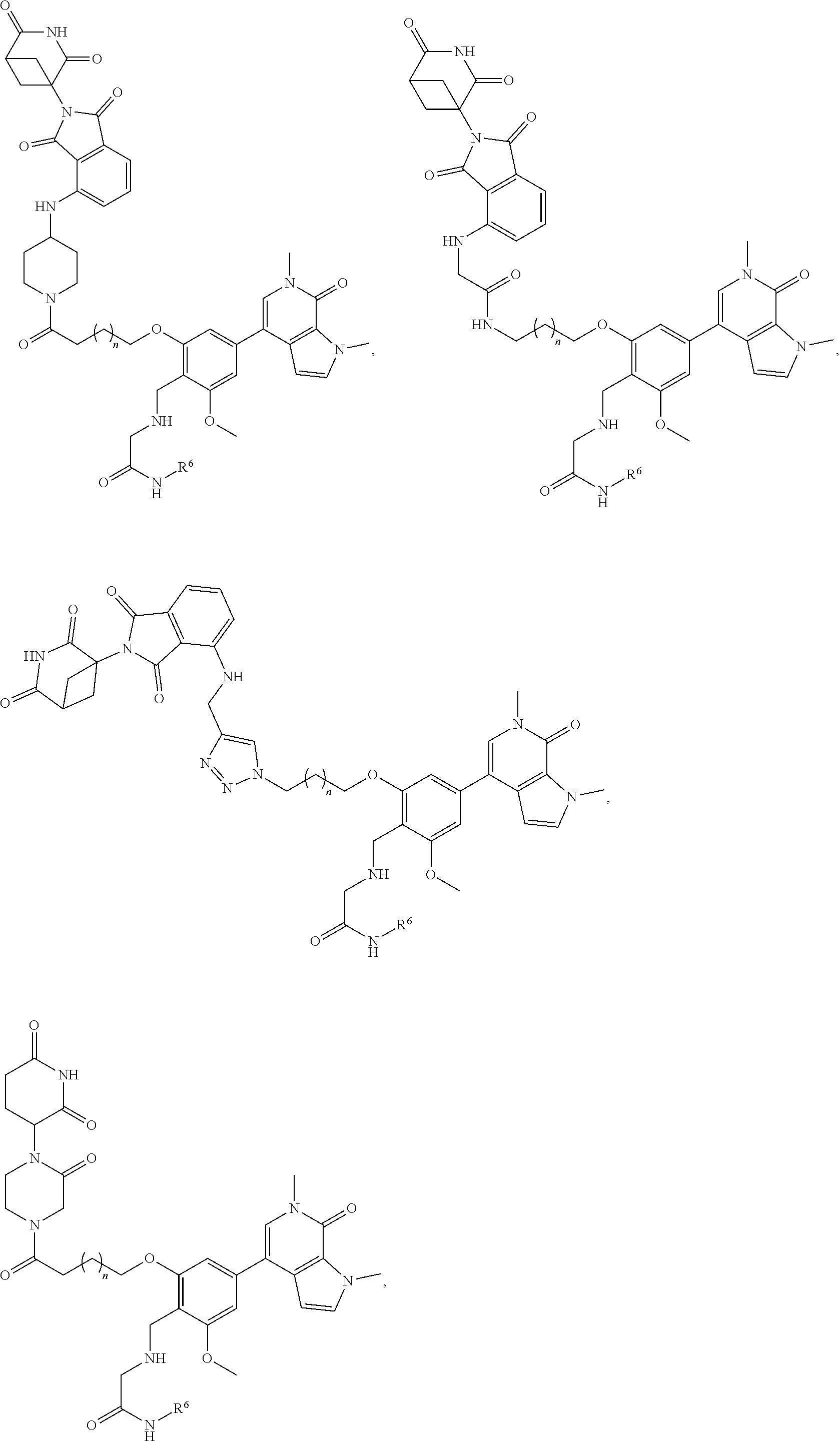

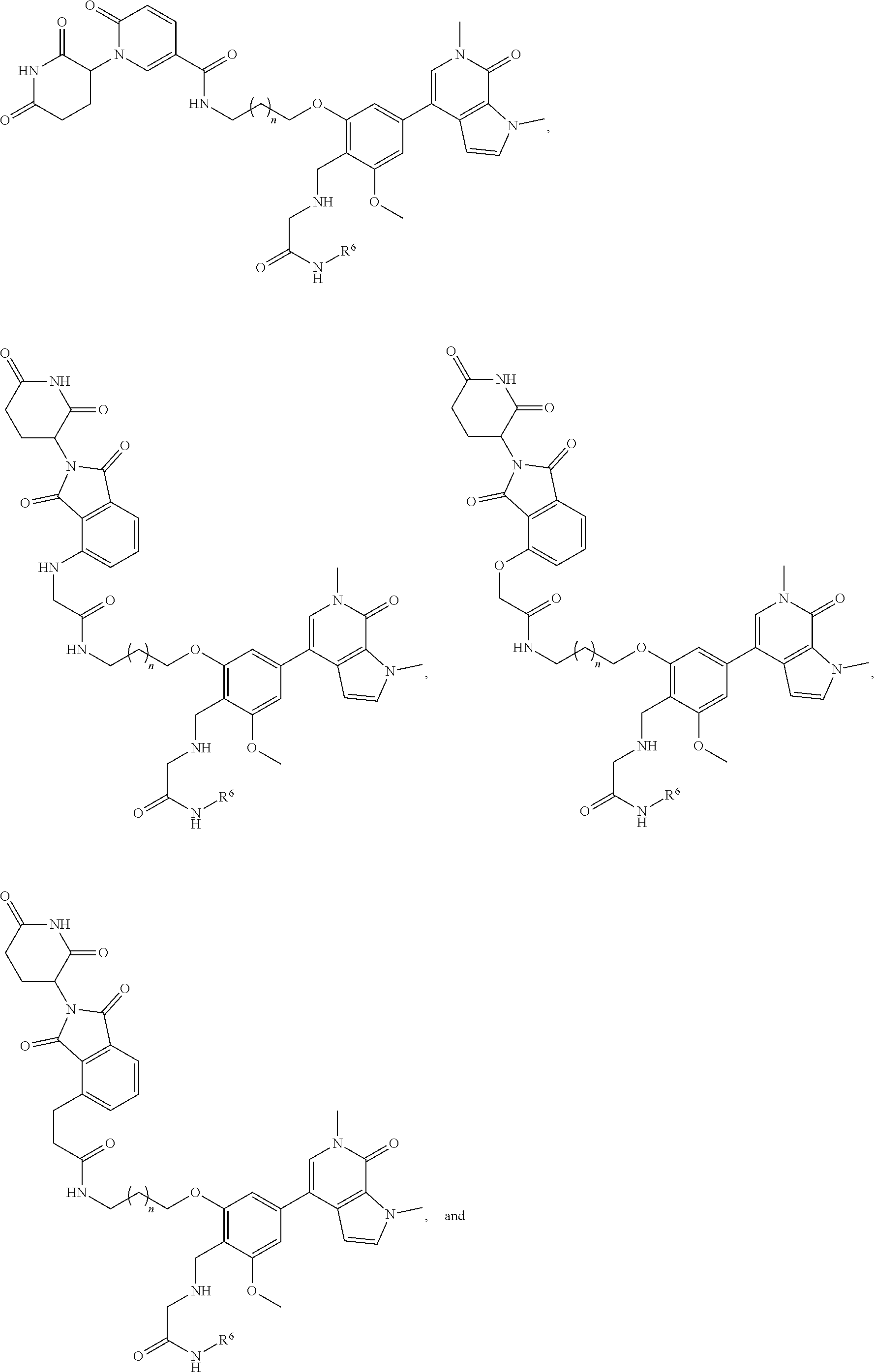

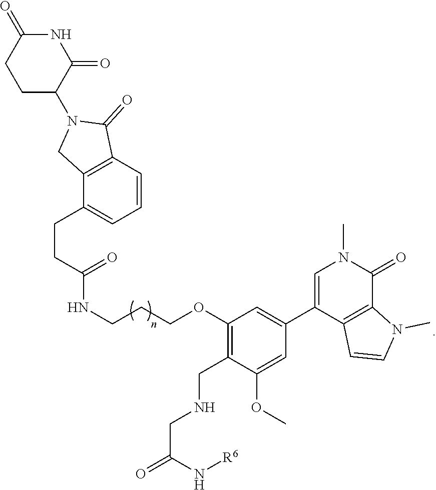

In some embodiments, the compound of Formula II is selected from:

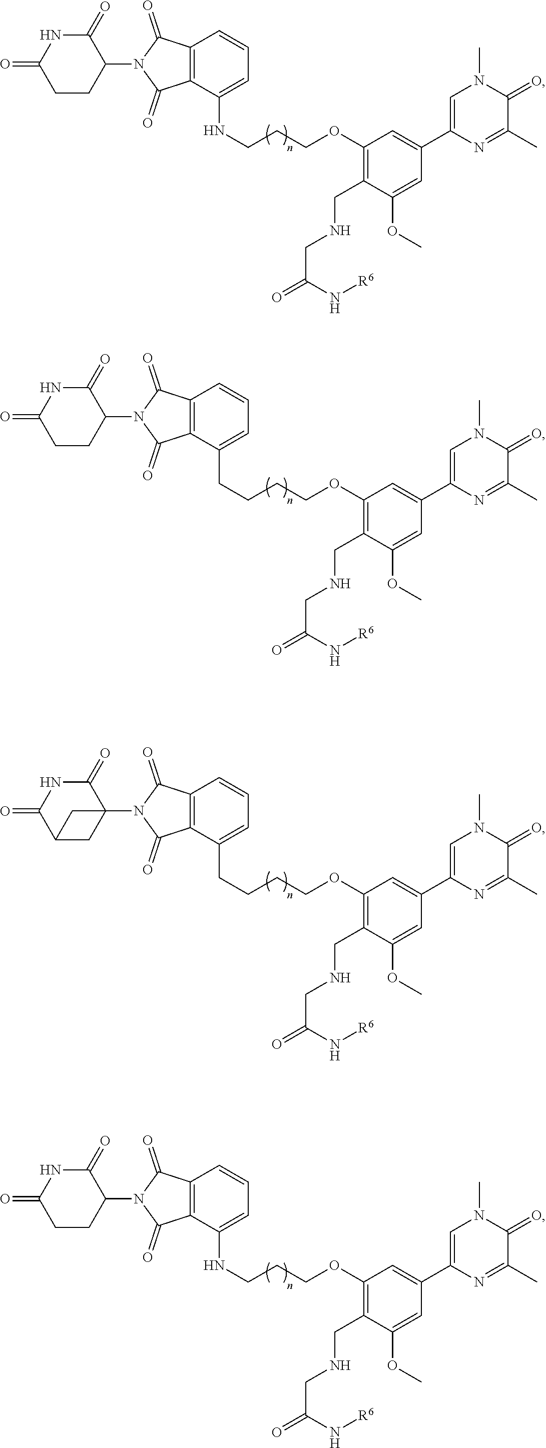

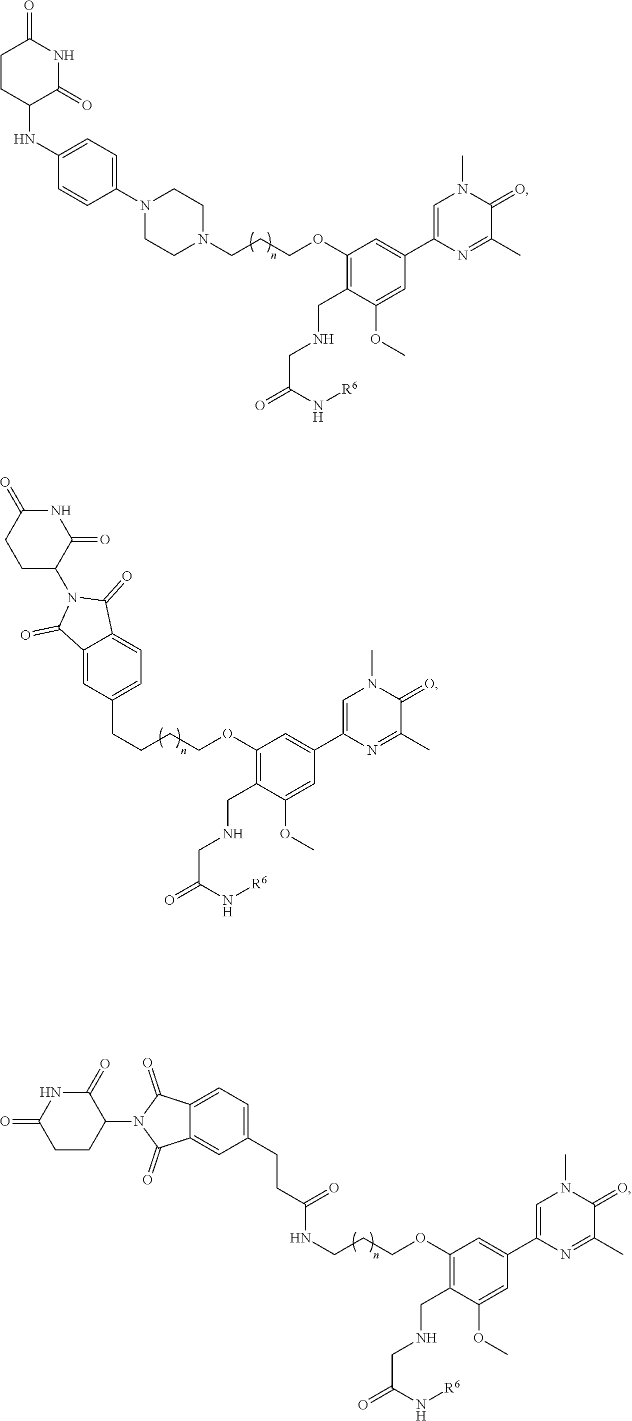

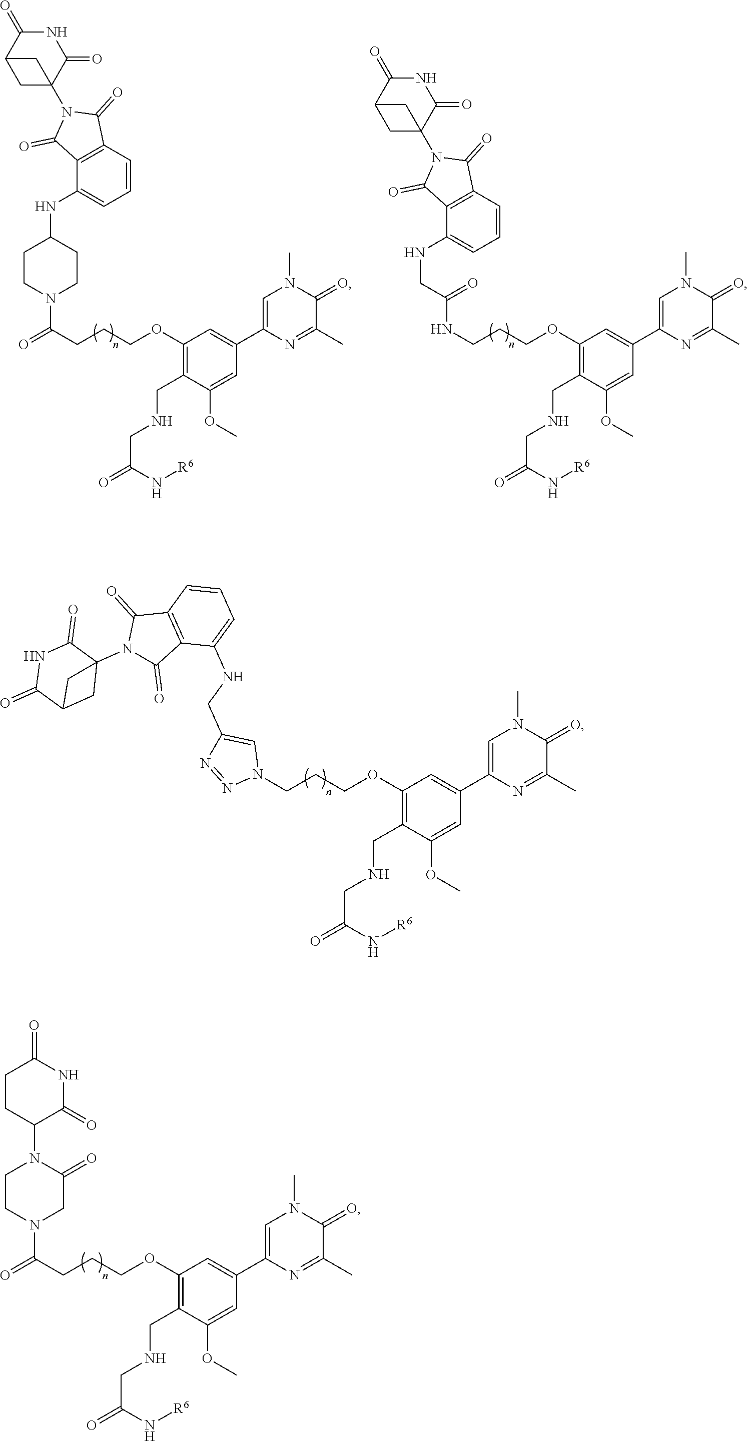

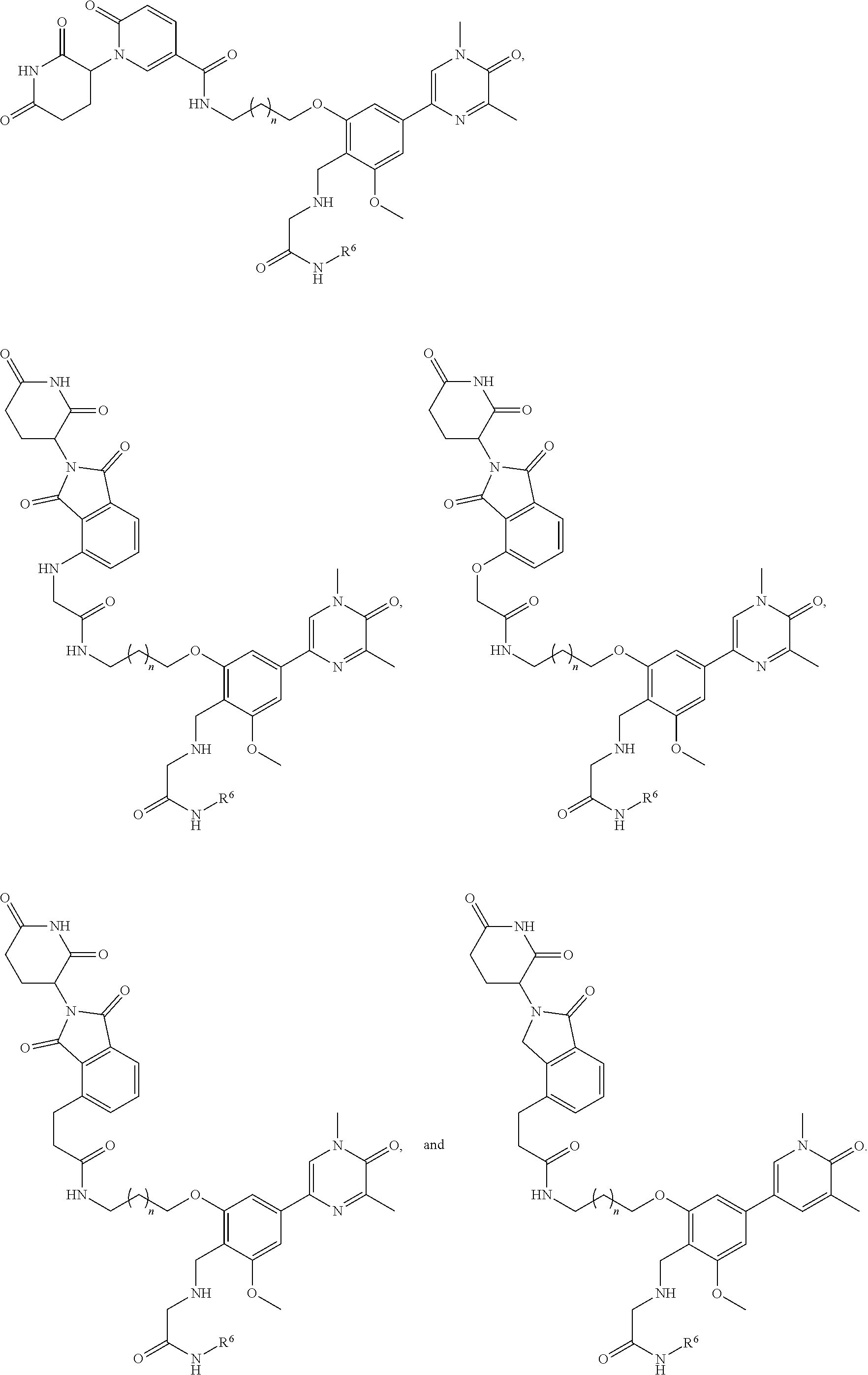

In some embodiments of Formula II,

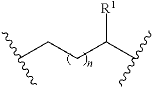

is selected from:

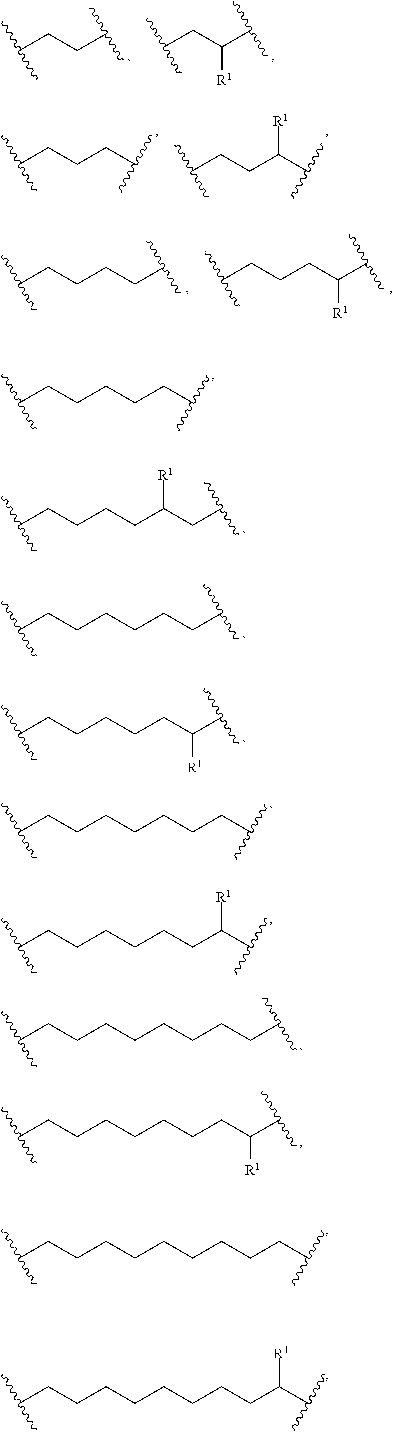

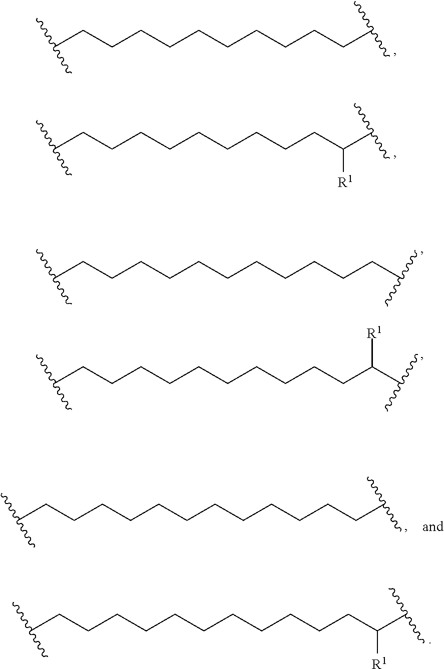

### Non-Limiting Examples of BRD9-Binding Compounds of Formula III

In some embodiments, the compound of Formula III is selected from:

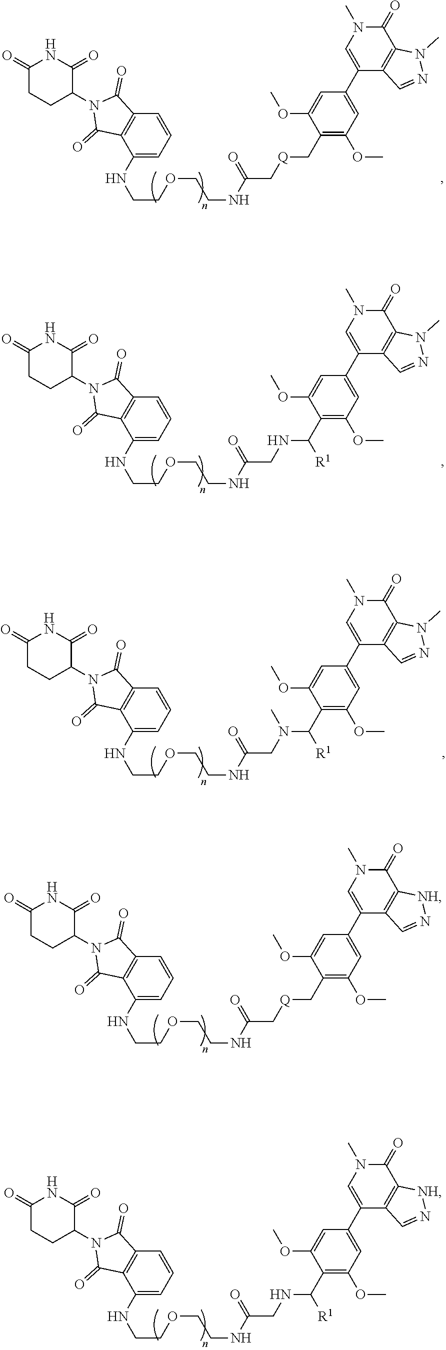

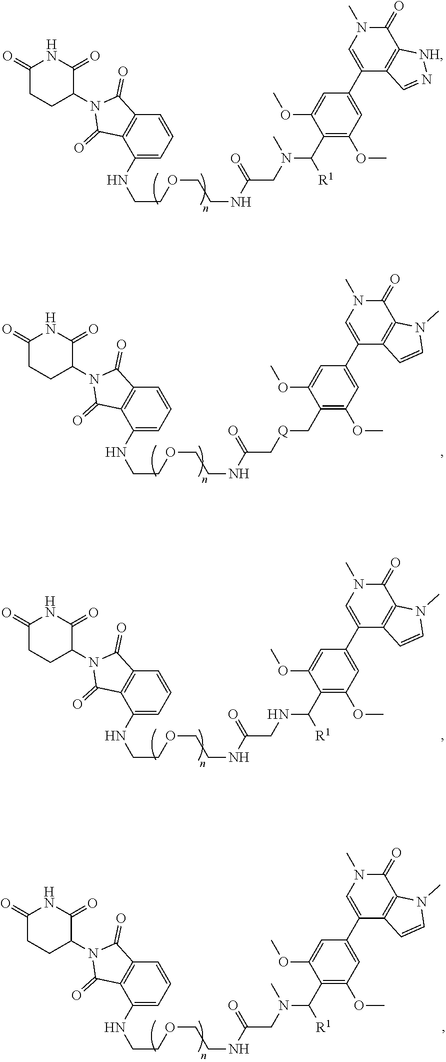

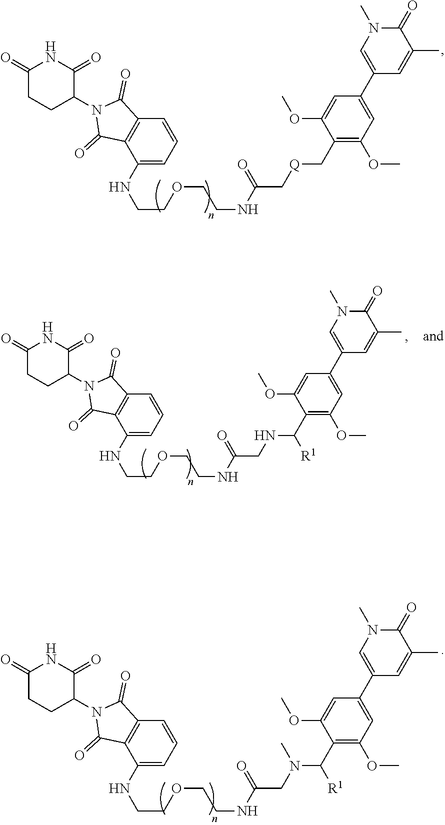

In some embodiments, the compound of Formula III is selected from:

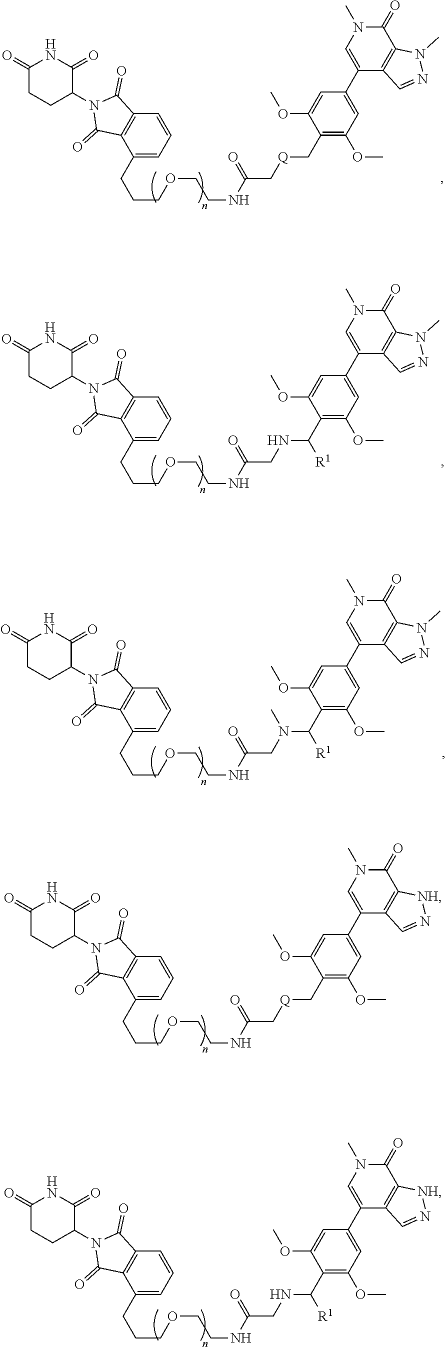

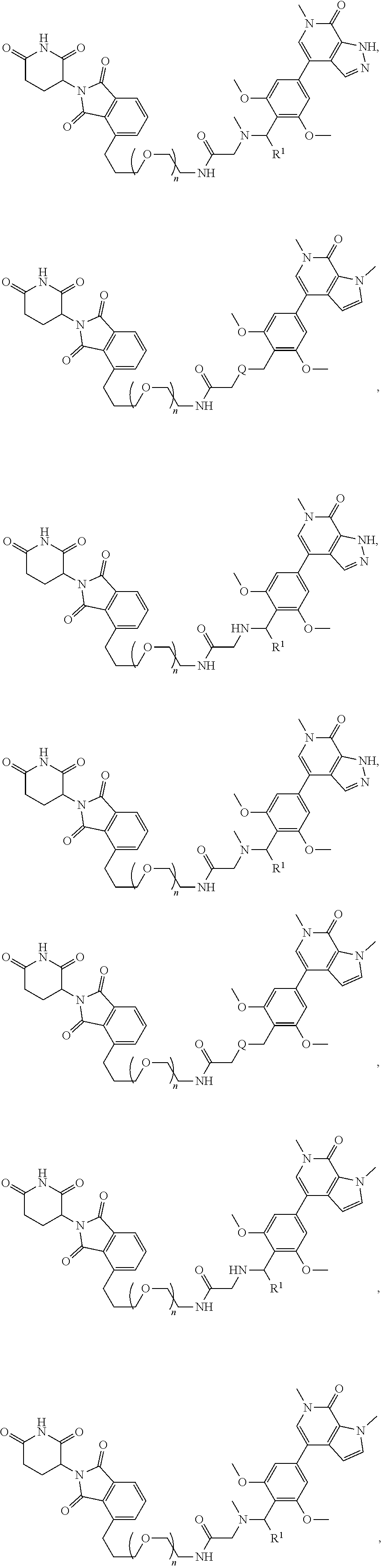

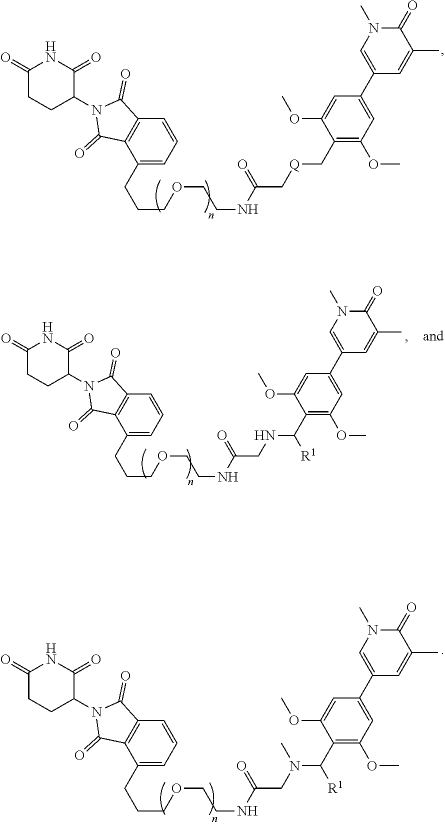

In some embodiments, the compound of Formula III is selected from:

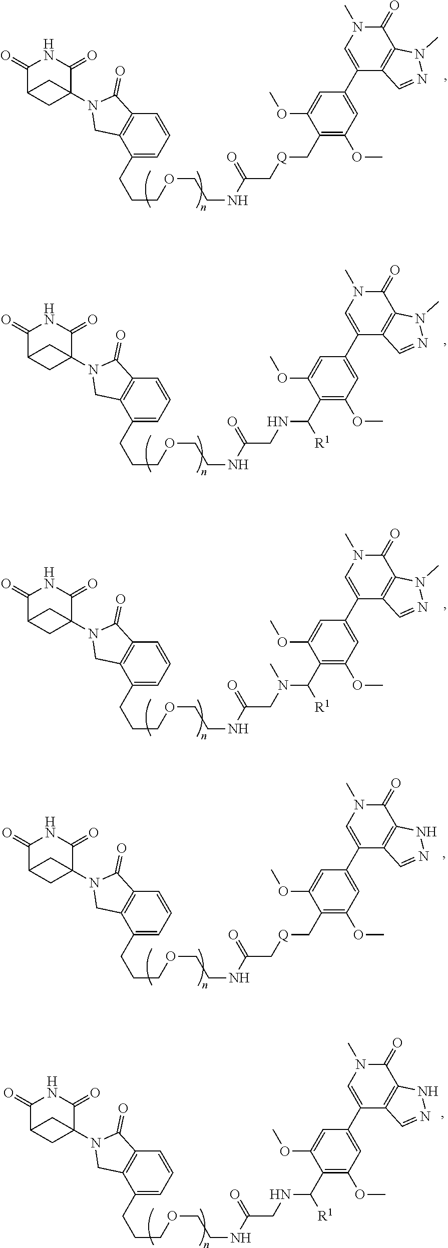

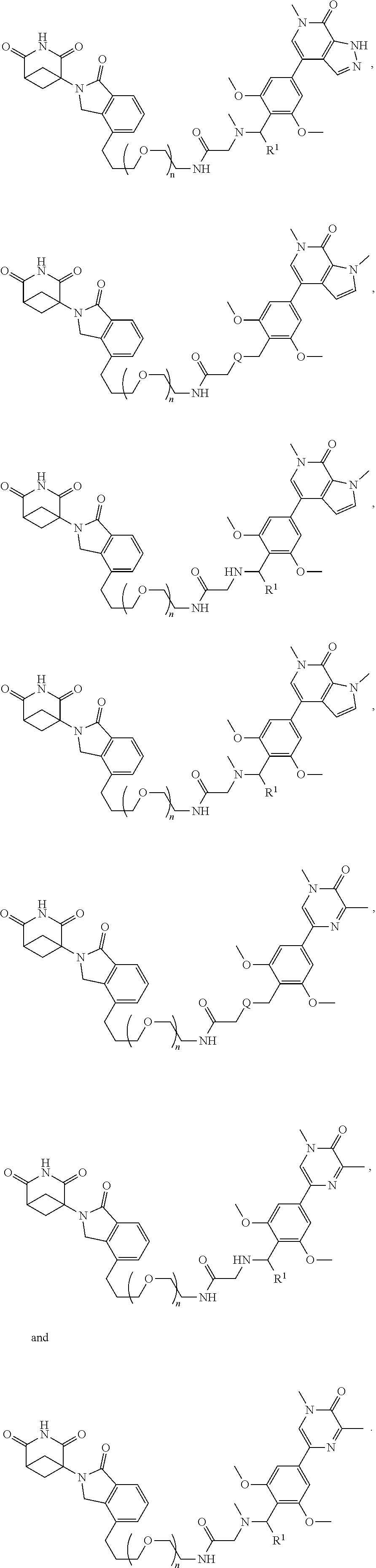

In some embodiments, the compound of Formula III is selected from:

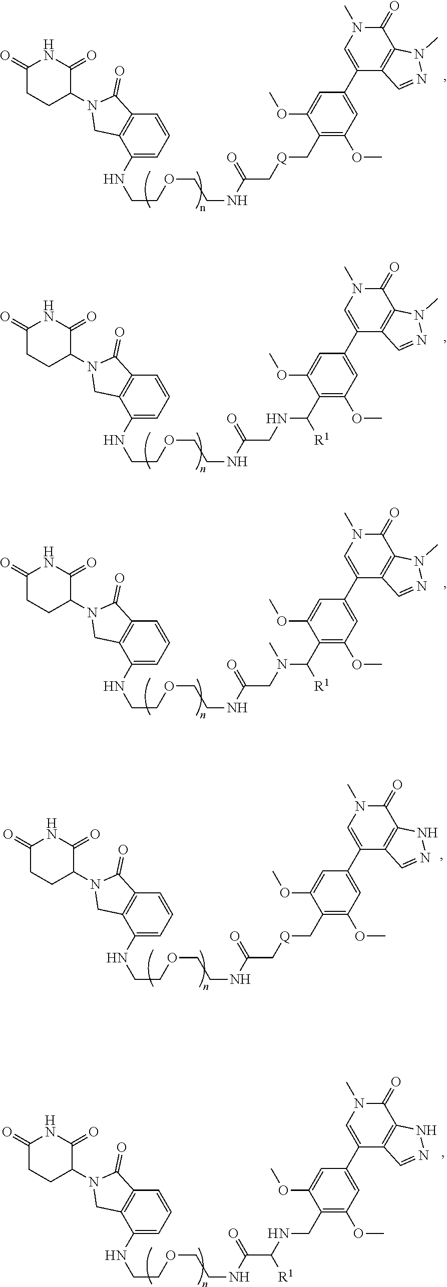

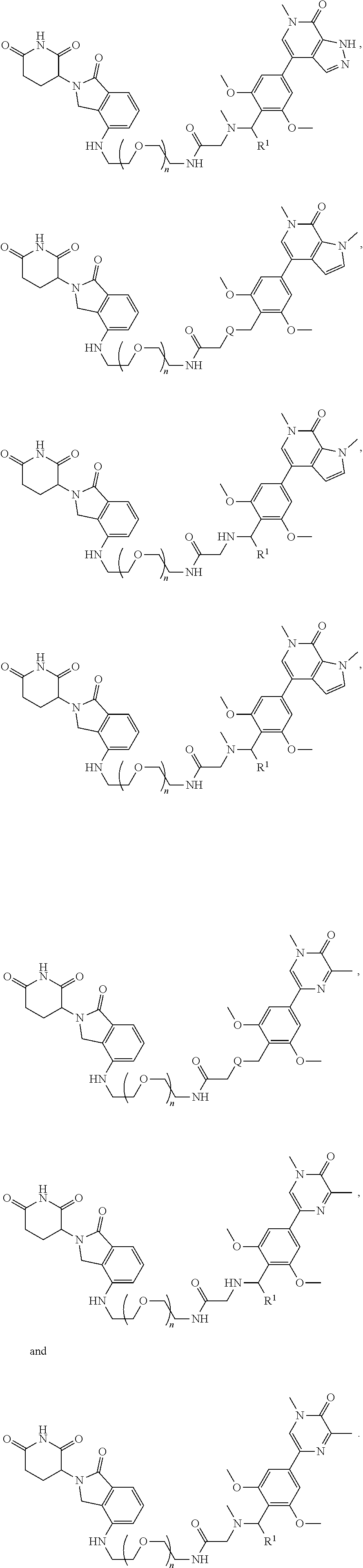

In some embodiments, the compound of Formula III is selected from:

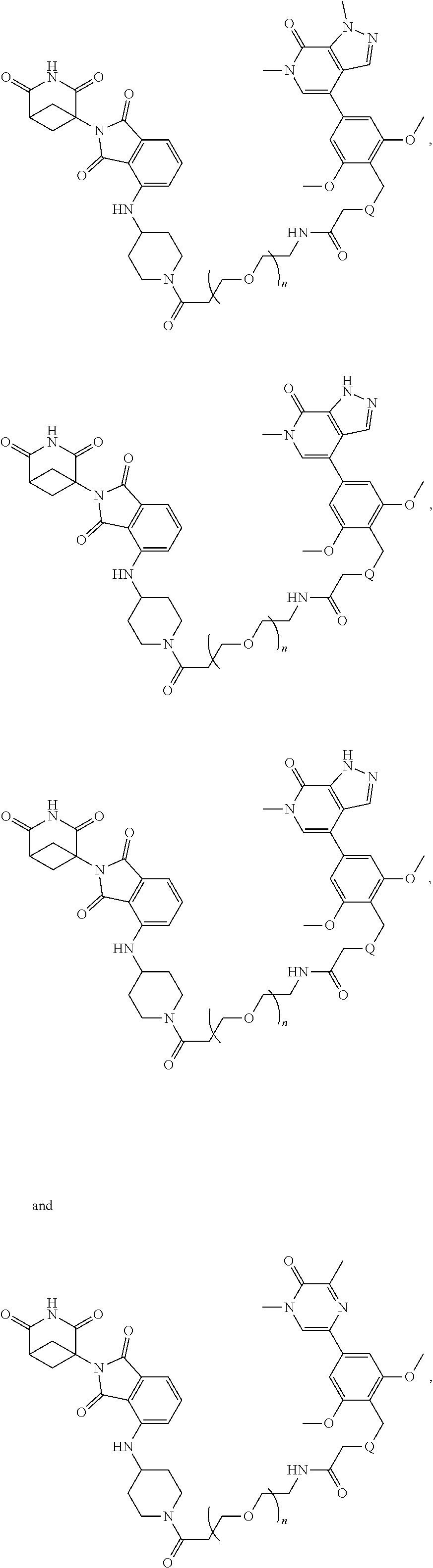

In some embodiments, the compound of Formula III is selected from:

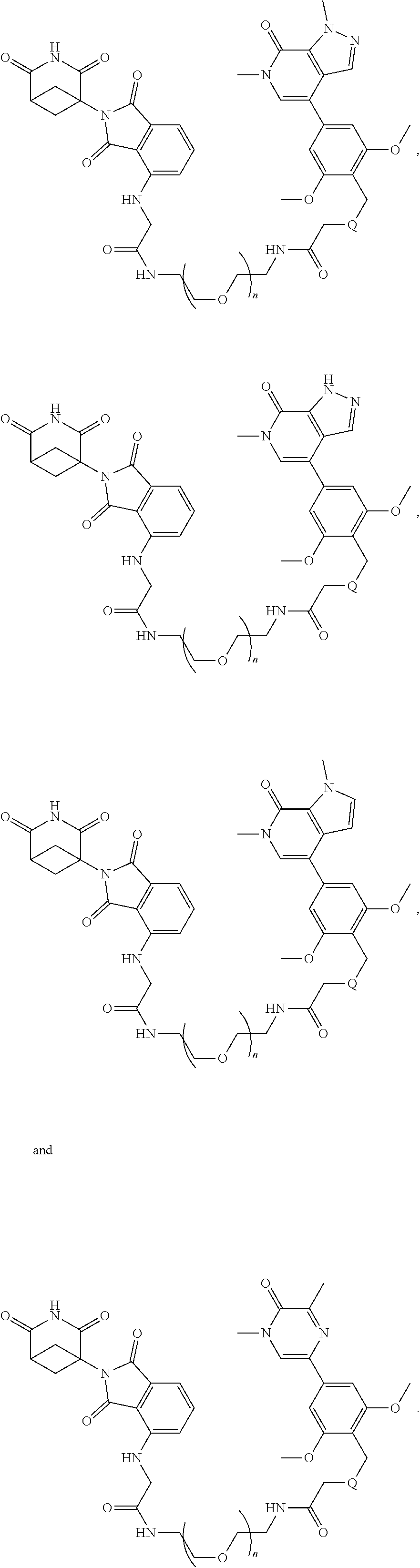

In some embodiments, the compound of Formula III is selected from:

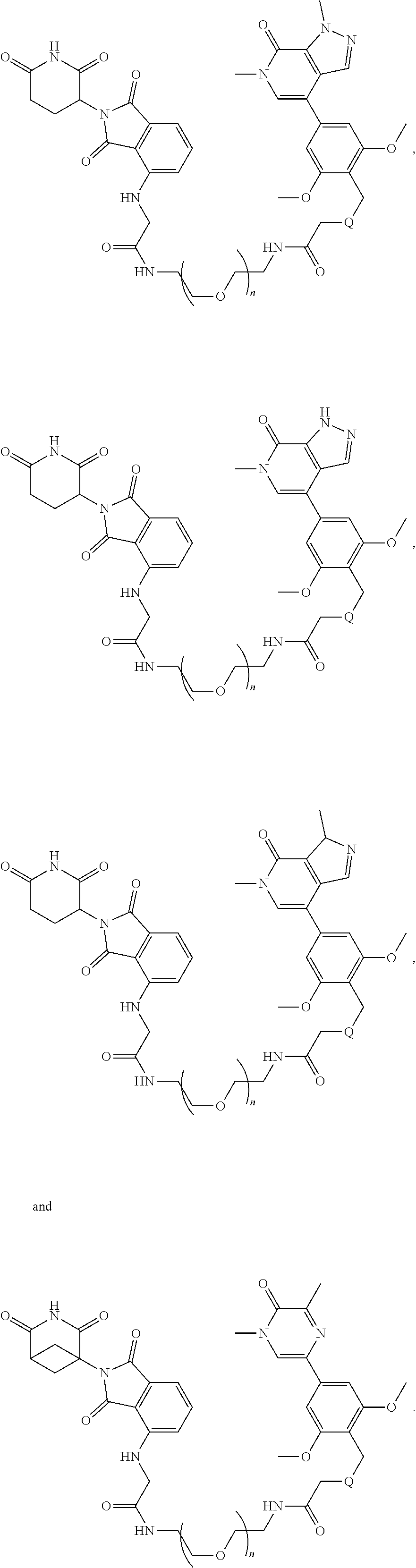

In some embodiments, the compound of Formula III is selected from:

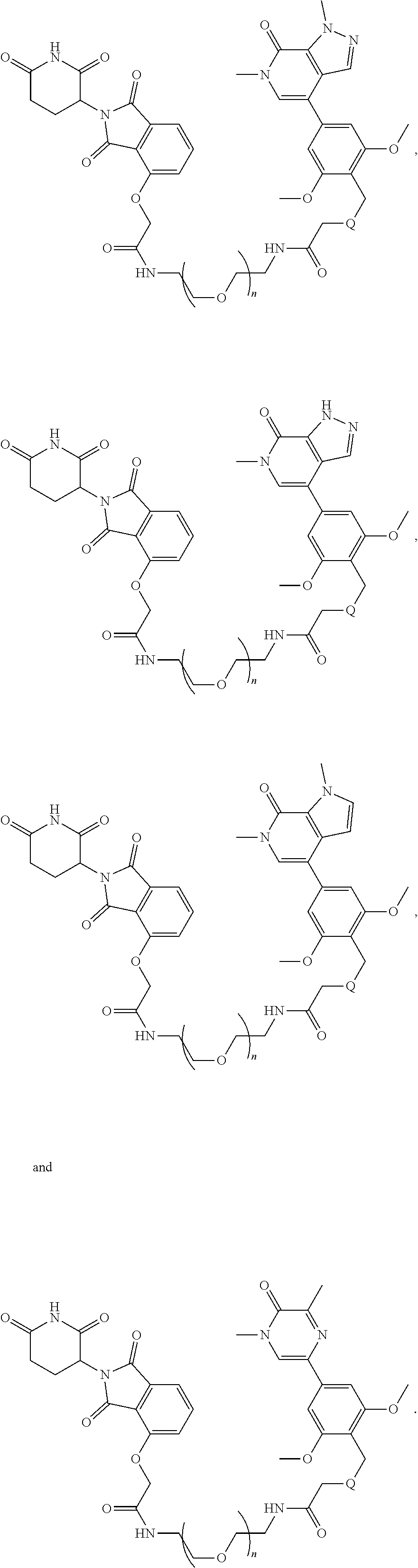

In some embodiments, the compound of Formula III is selected from:

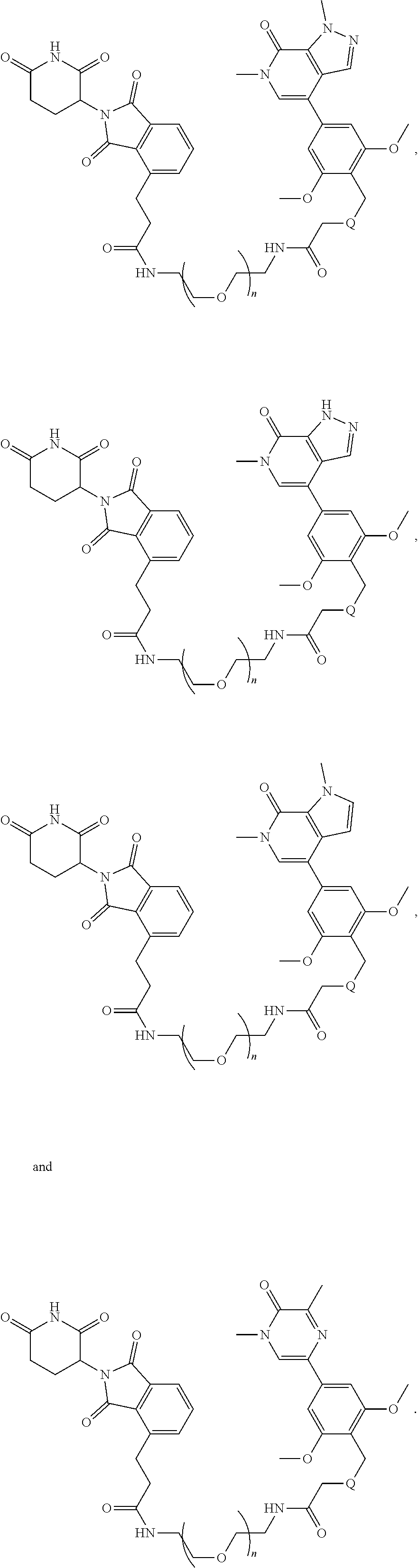

In some embodiments, the compound of Formula III is selected from:

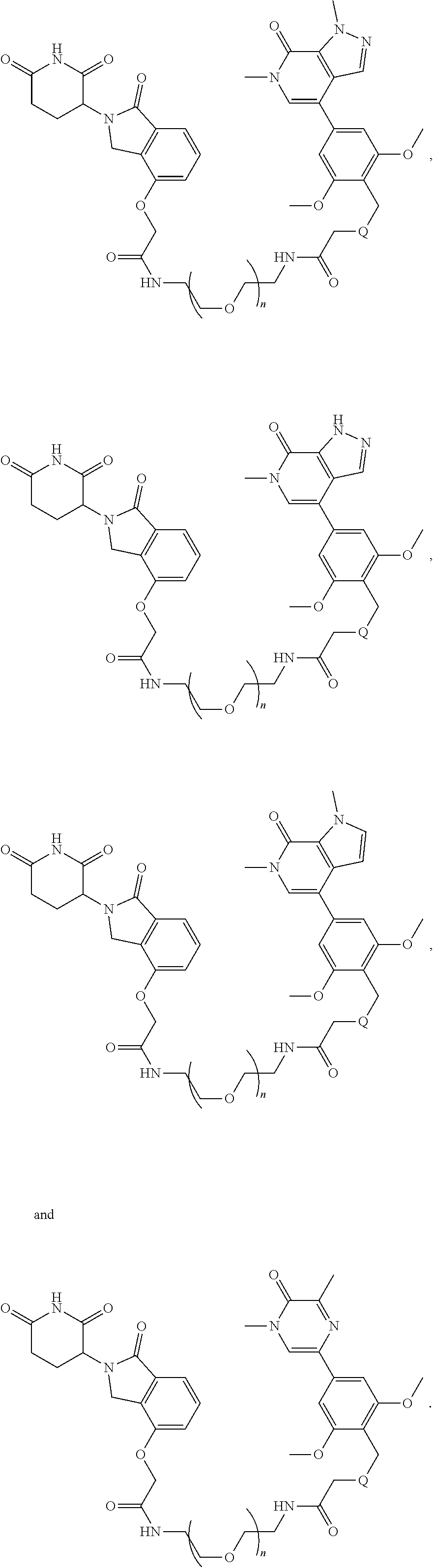

In some embodiments, the compound of Formula III is selected from:

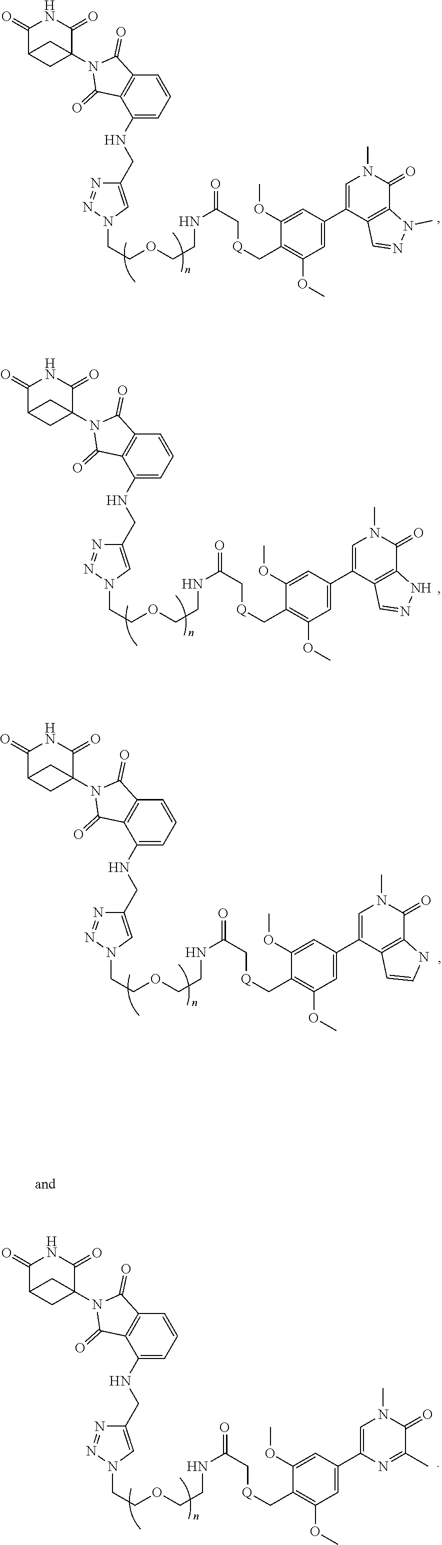

In some embodiments, the compound of Formula III is selected from:

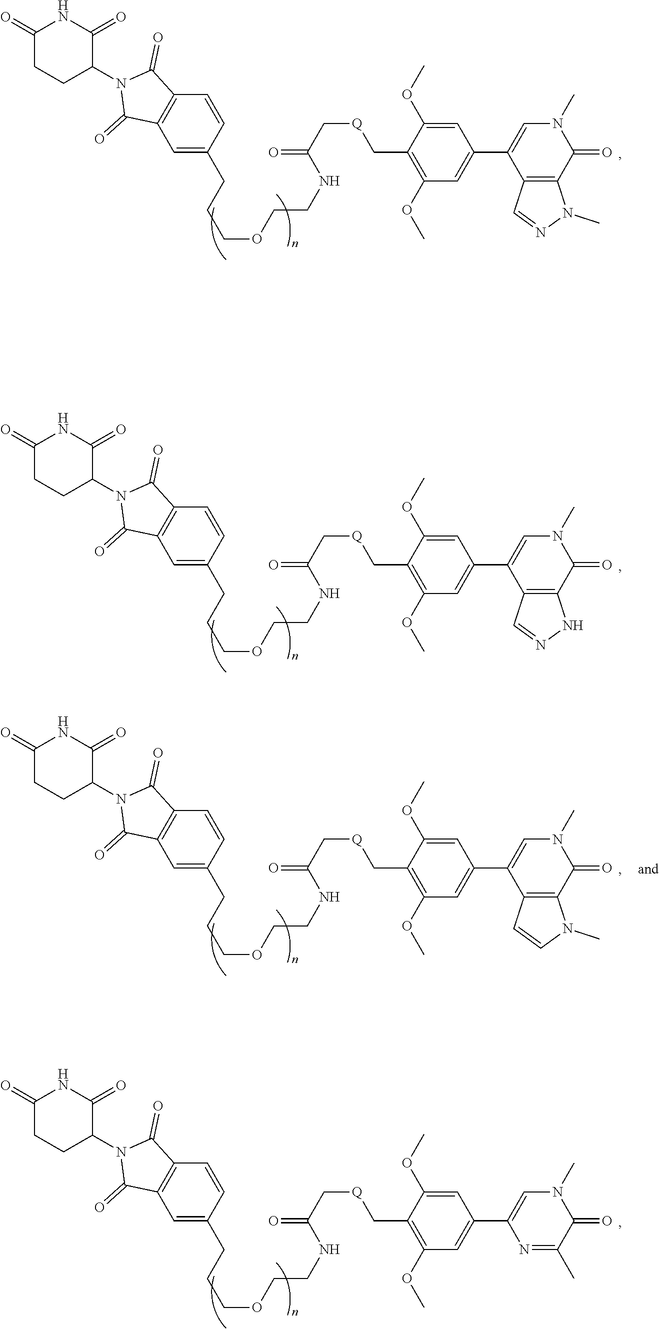

In some embodiments, the compound of Formula III is selected from:

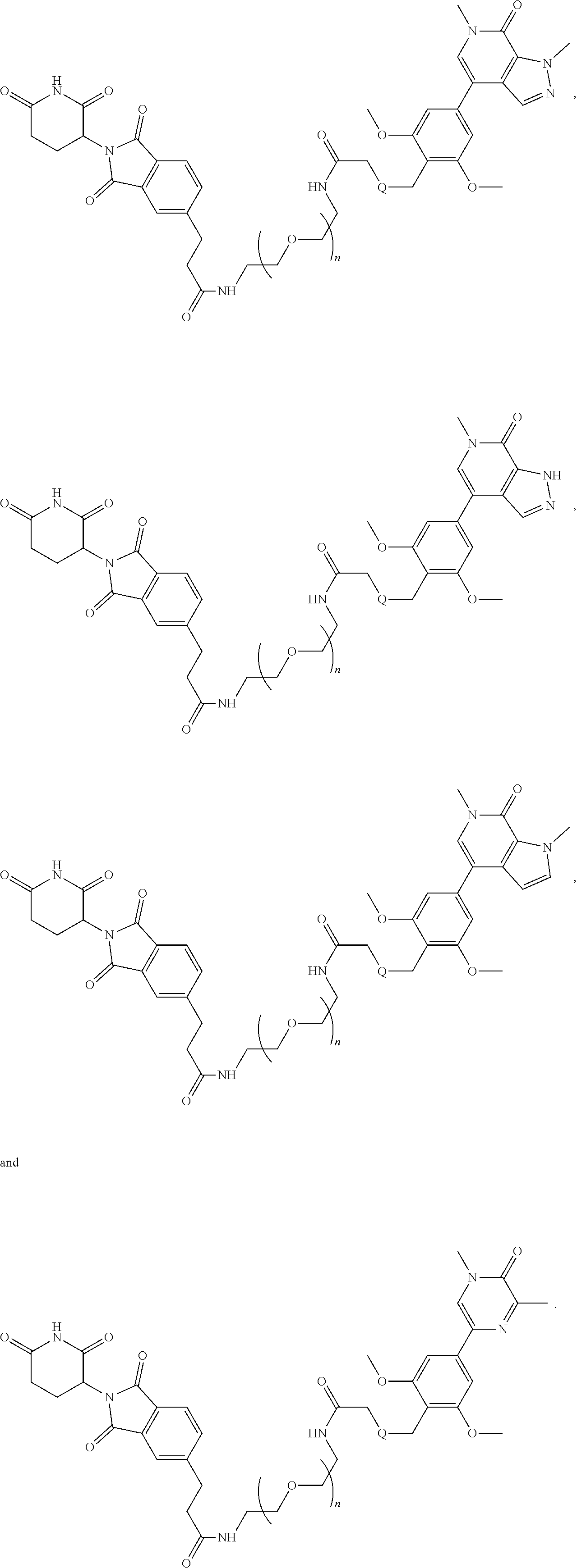

In some embodiments, the compound of Formula III is selected from:

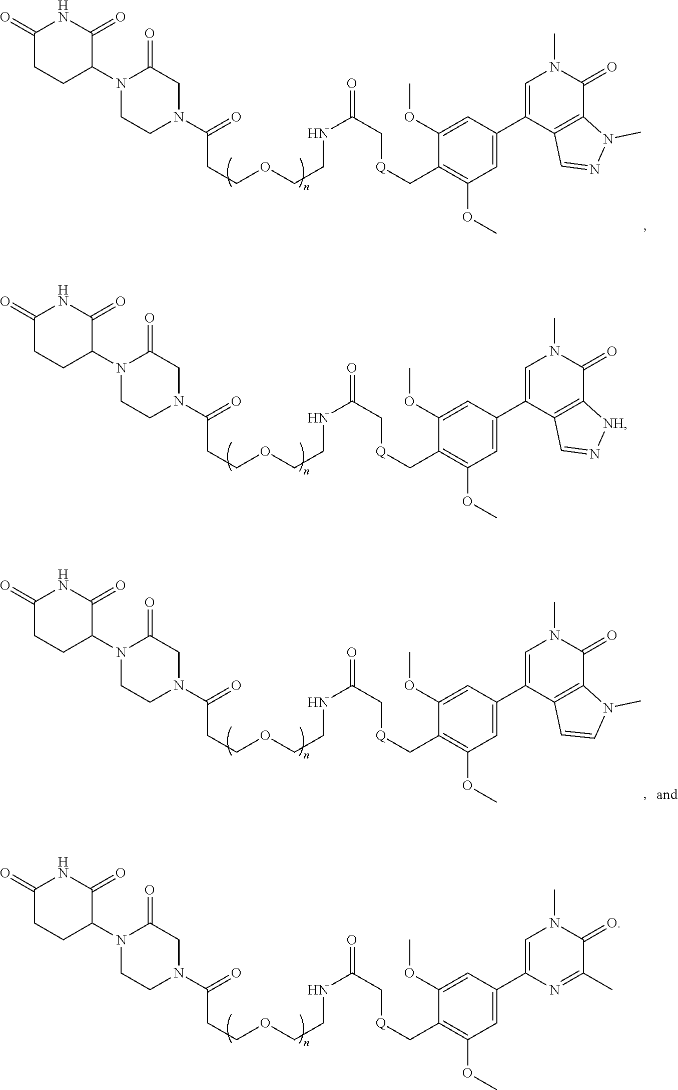

In some embodiments, the compound of Formula III is selected from:

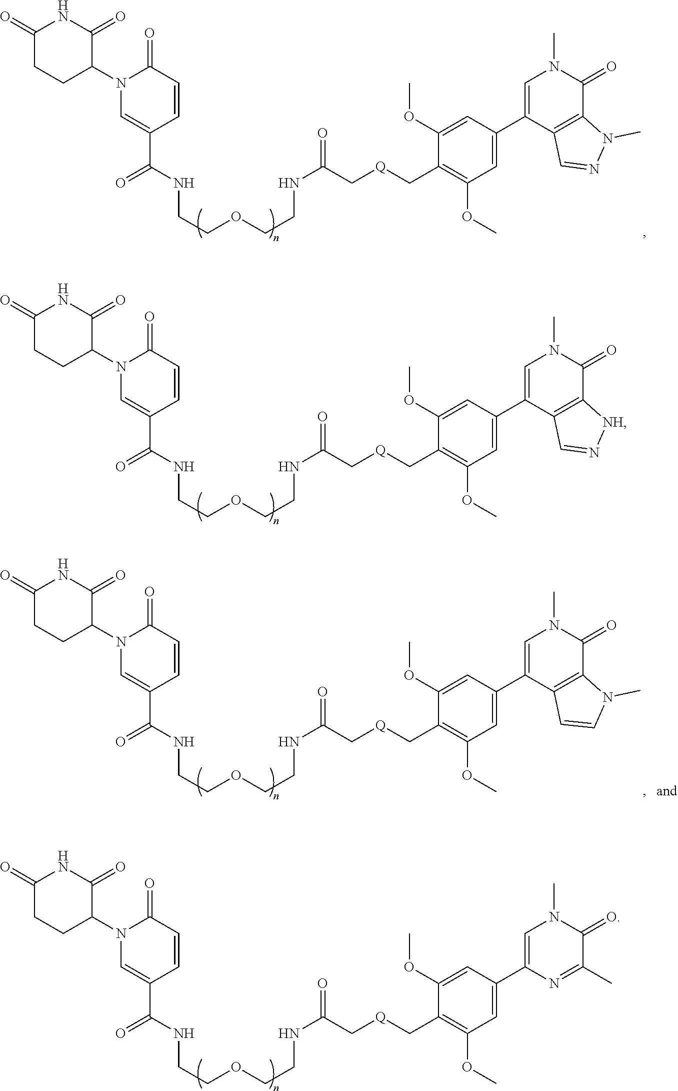

In some embodiments, the compound of Formula III is selected from:

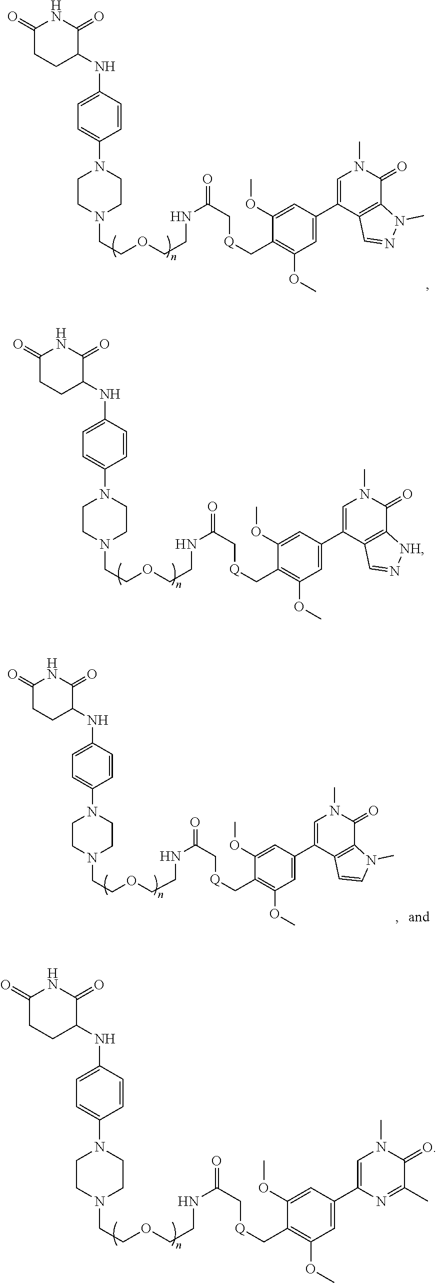

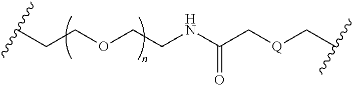

In some embodiments of Formula III, is selected from:

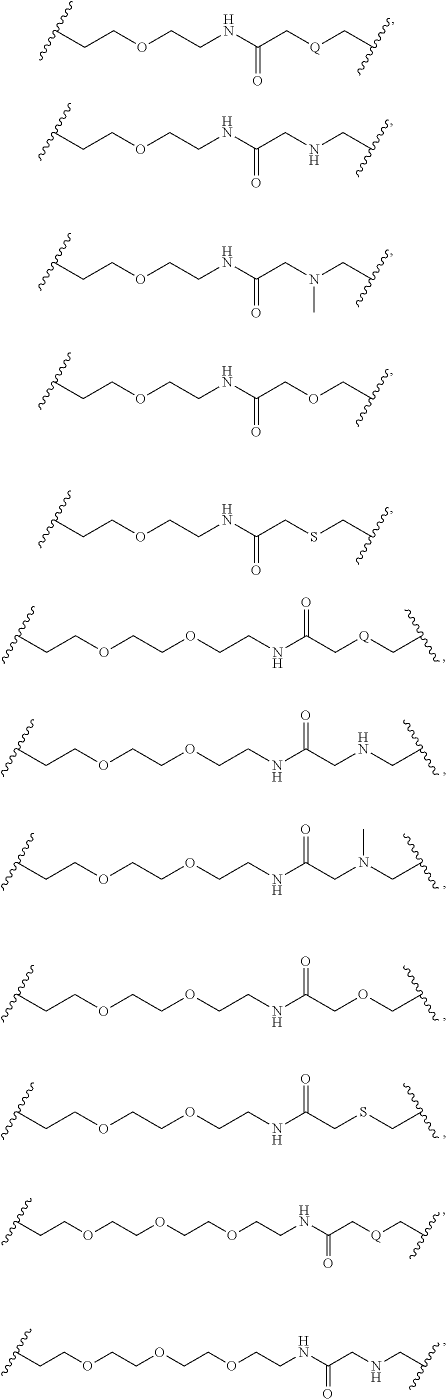

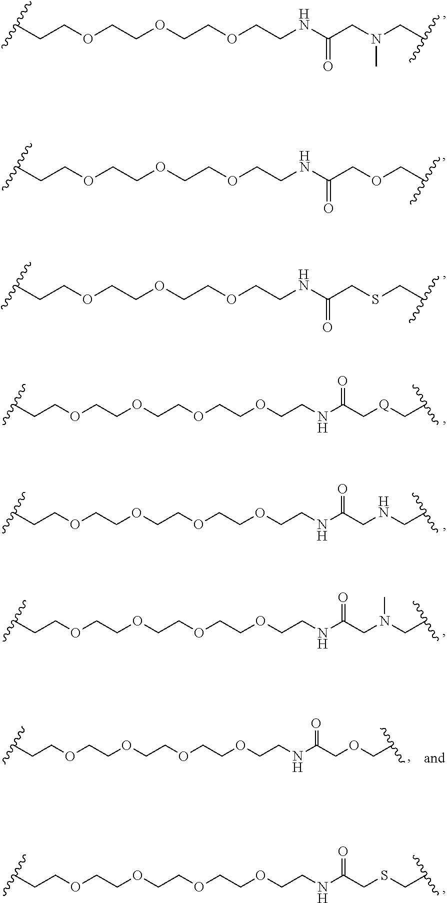

### Non-Limiting Examples of BRD9-Binding Compounds of Formula IV

In some embodiments, the compound of Formula IV is selected from:

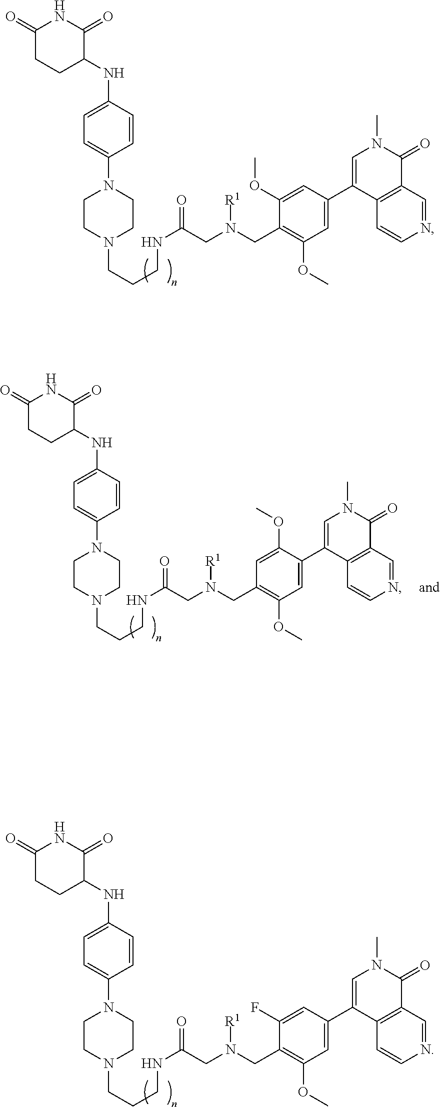

In some embodiments, the compound of Formula IV is selected from:

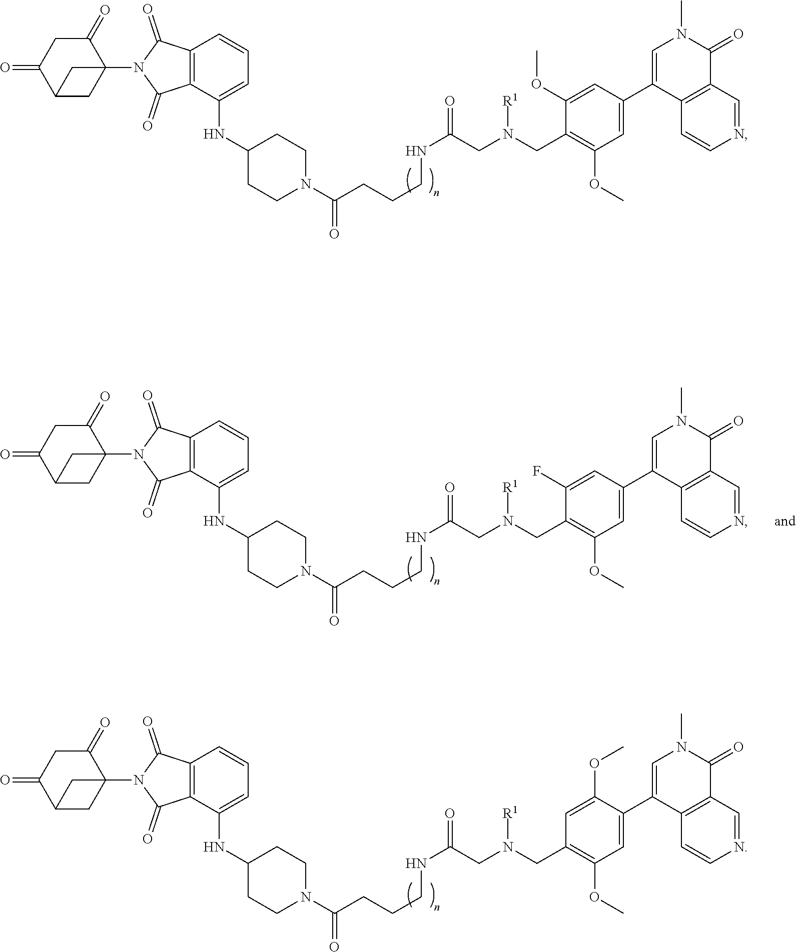

In some embodiments, the compound of Formula IV is selected from:

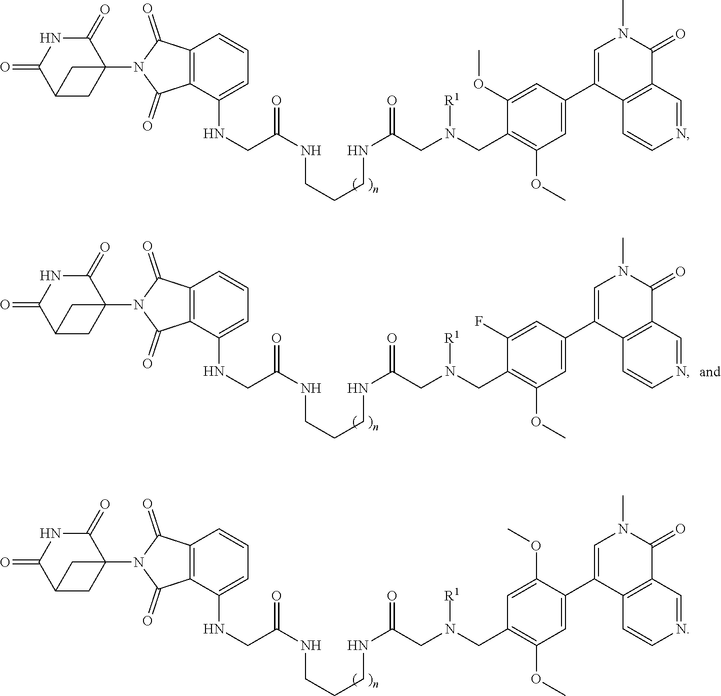

In some embodiments, the compound of Formula IV is selected from:

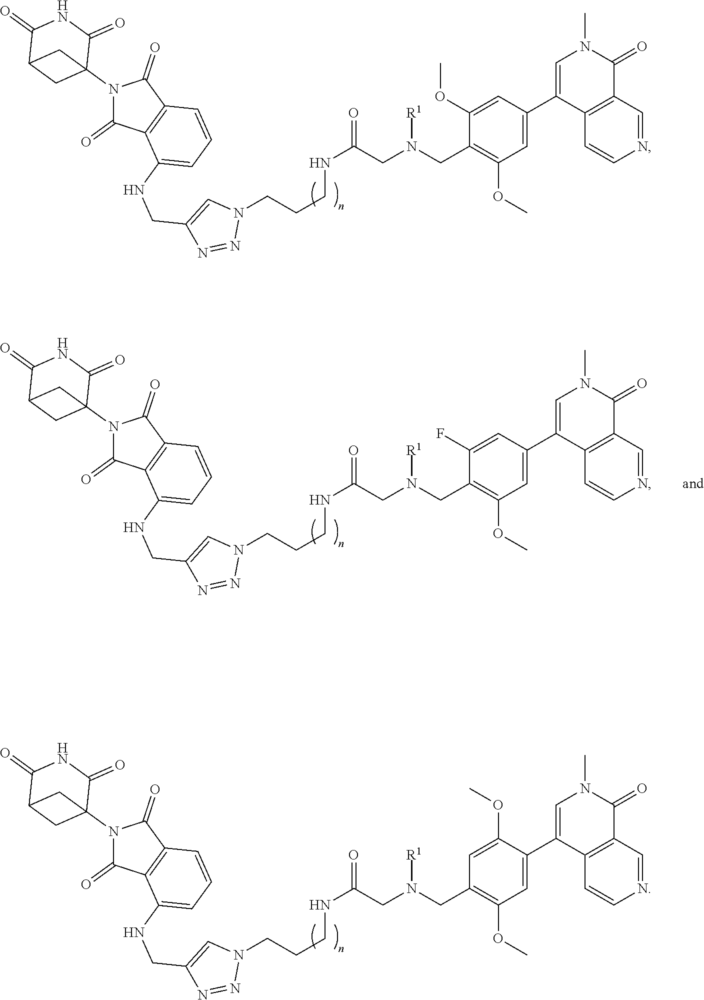

In some embodiments, the compound of Formula IV is selected from:

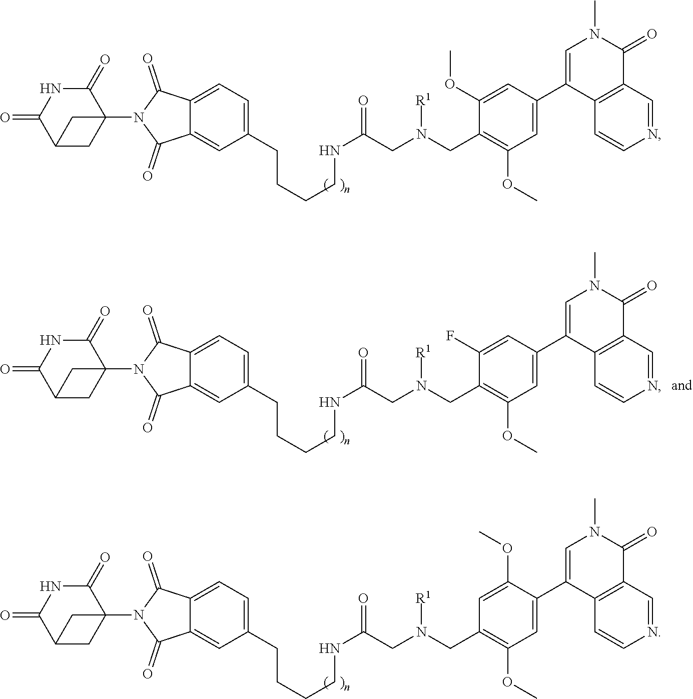

In some embodiments, the compound of Formula IV is selected from:

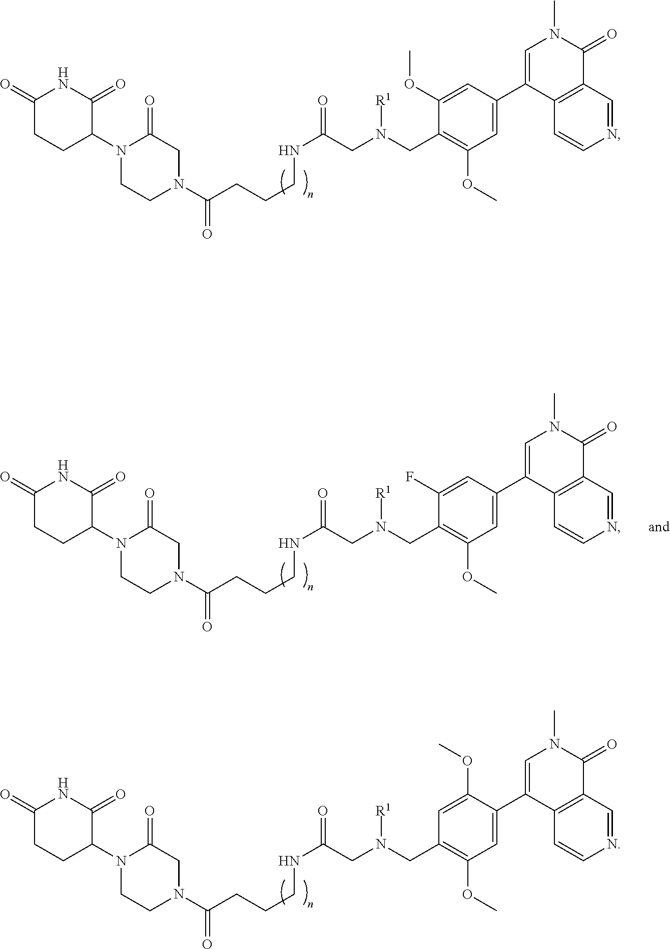

In some embodiments, the compound of Formula IV is selected from:

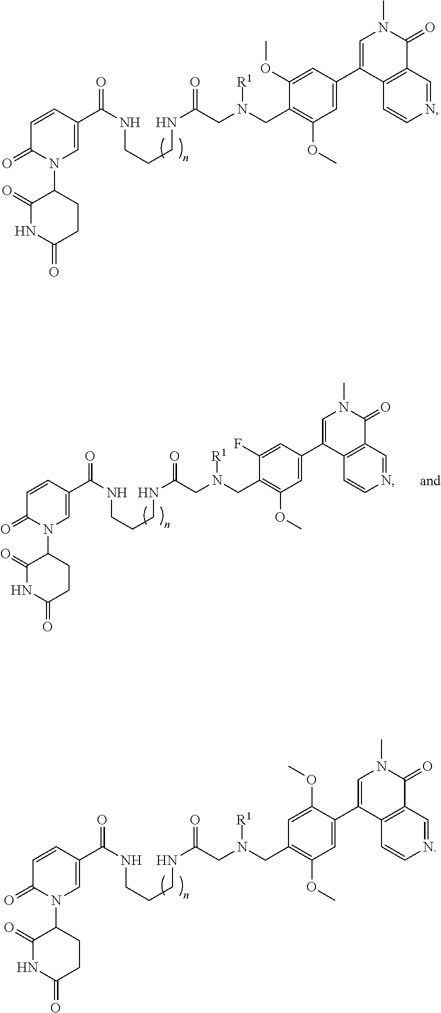

In some embodiments of Formula IV,

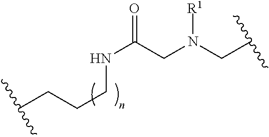

is selected from:

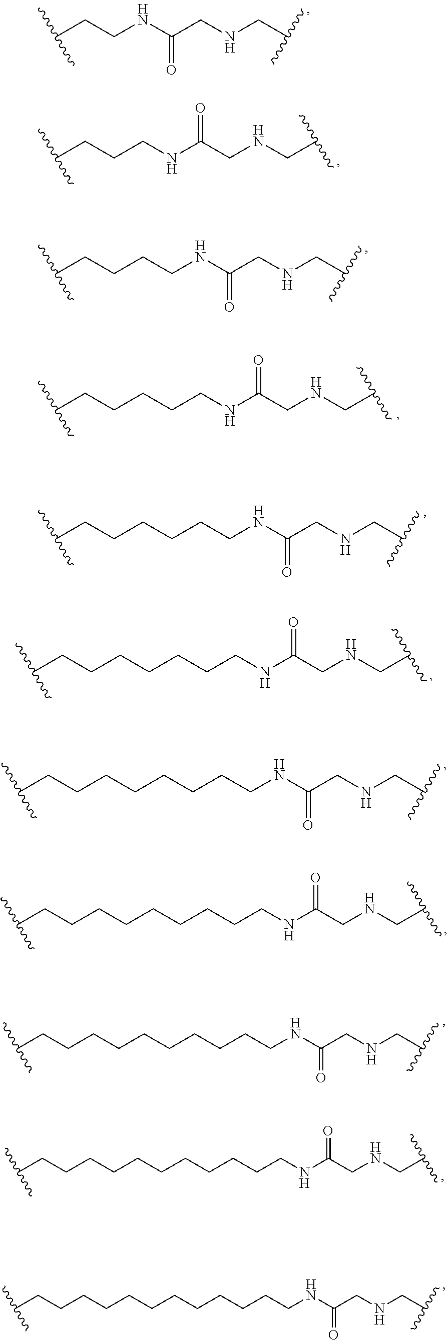

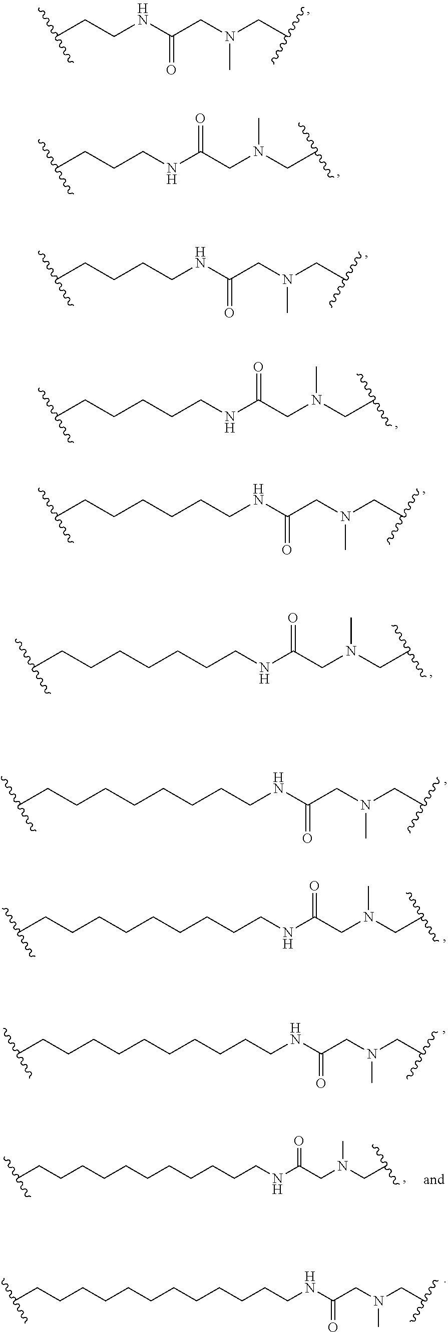

### Non-Limiting Examples of MTH1-Binding Compounds of Formula V

In some embodiments, a compound of Formula V is selected from:

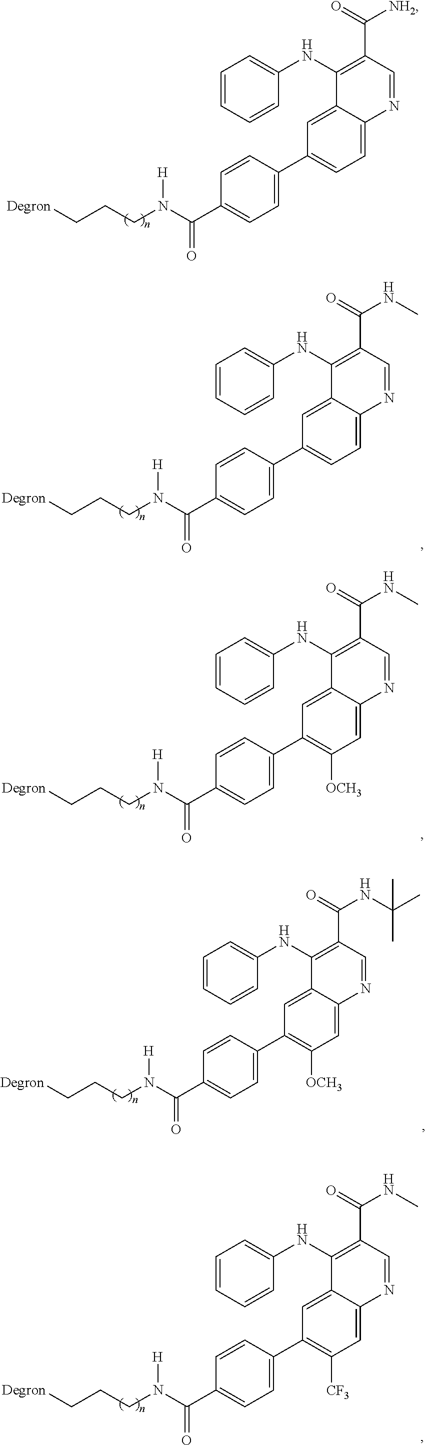

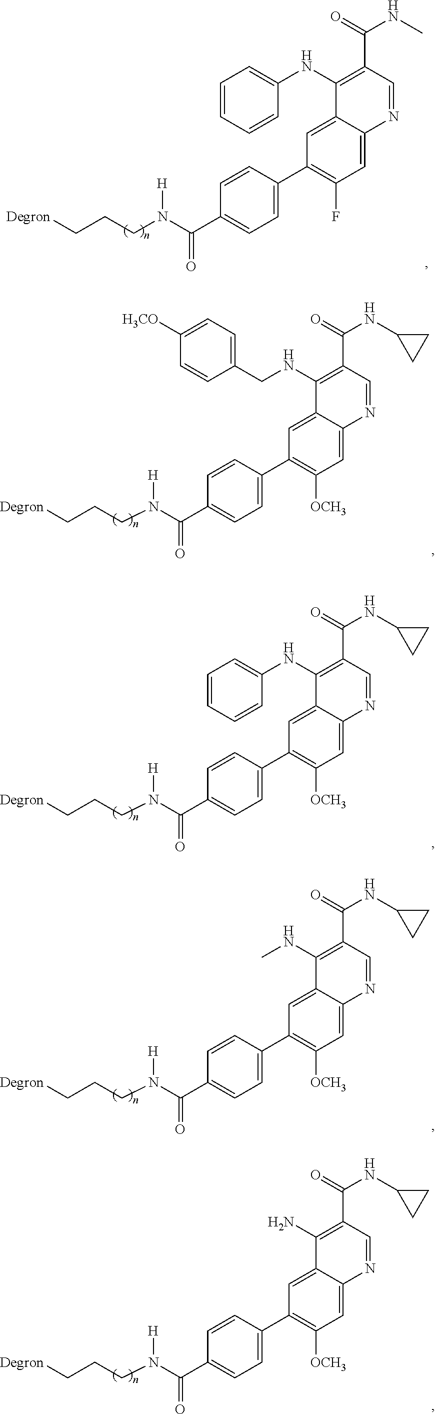

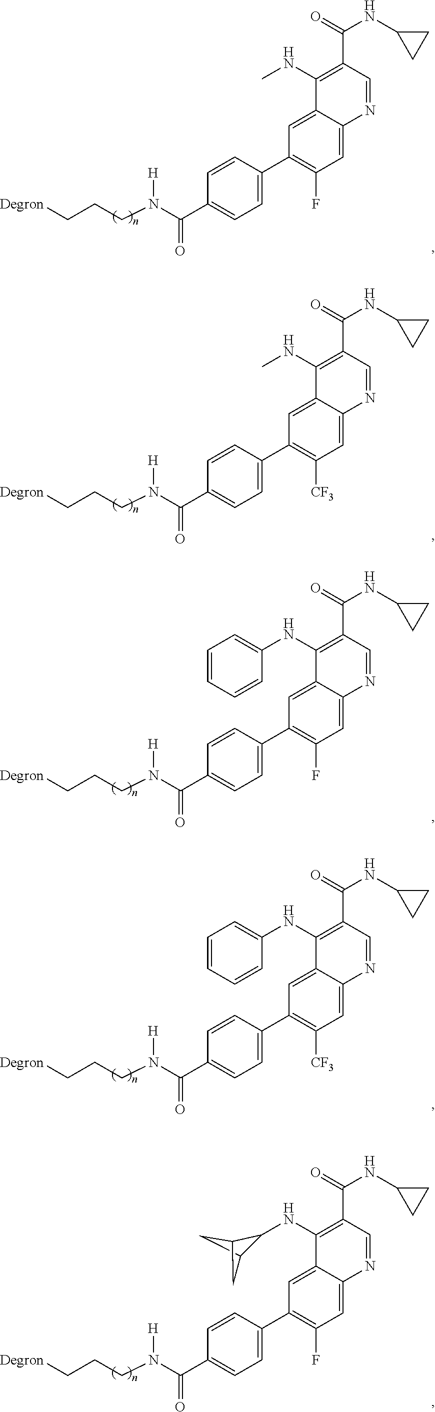

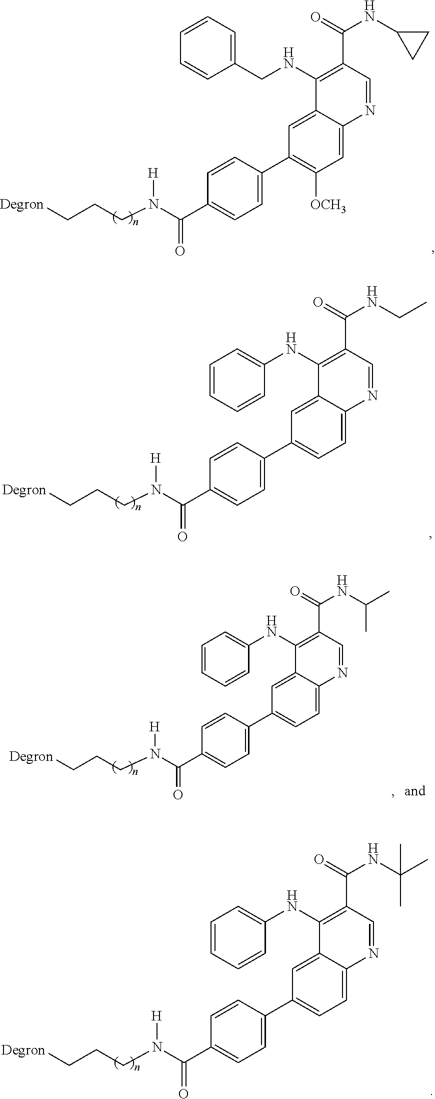

In some embodiments, a compound of Formula V is selected from:

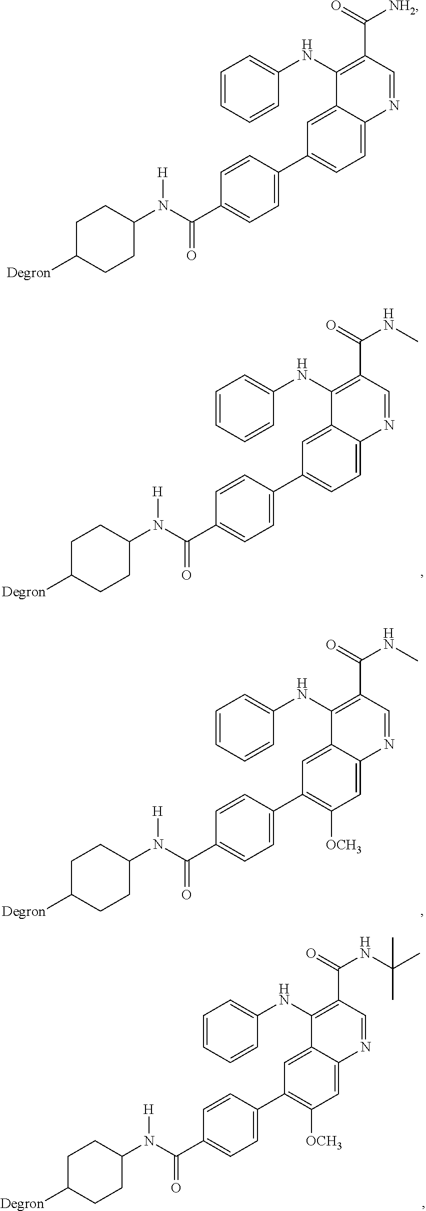

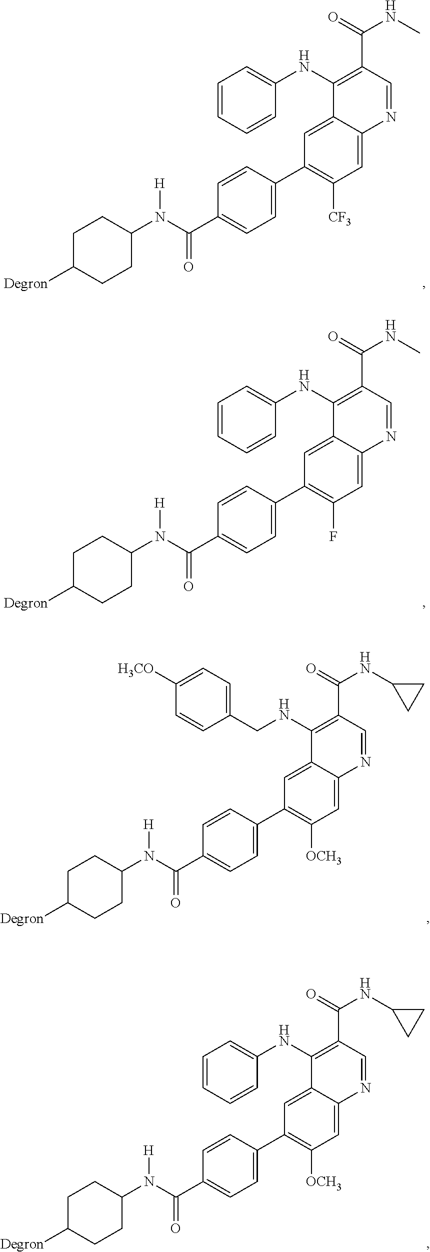

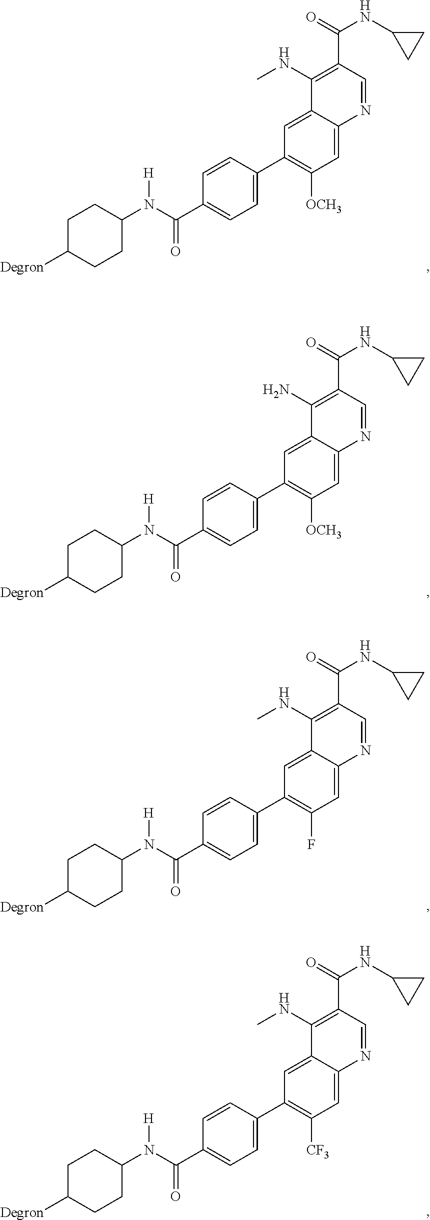

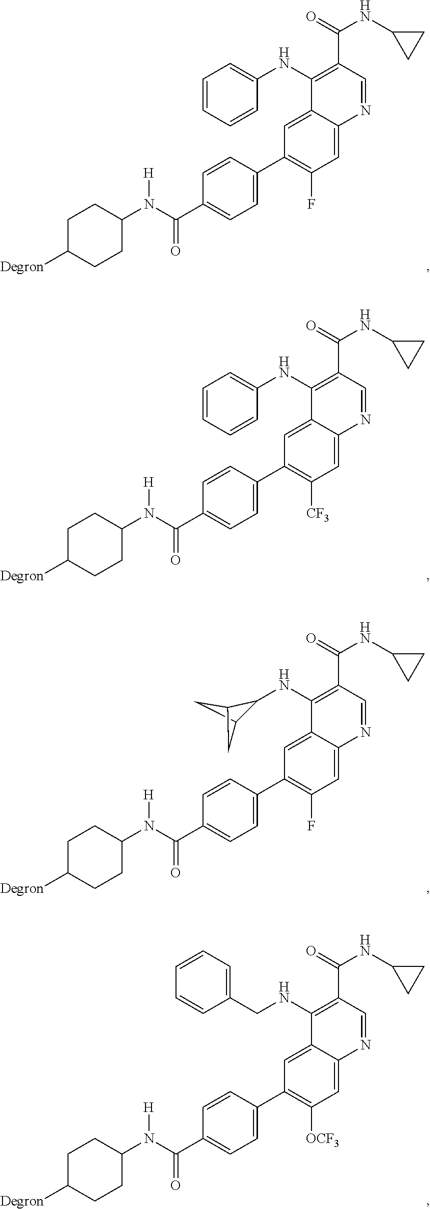

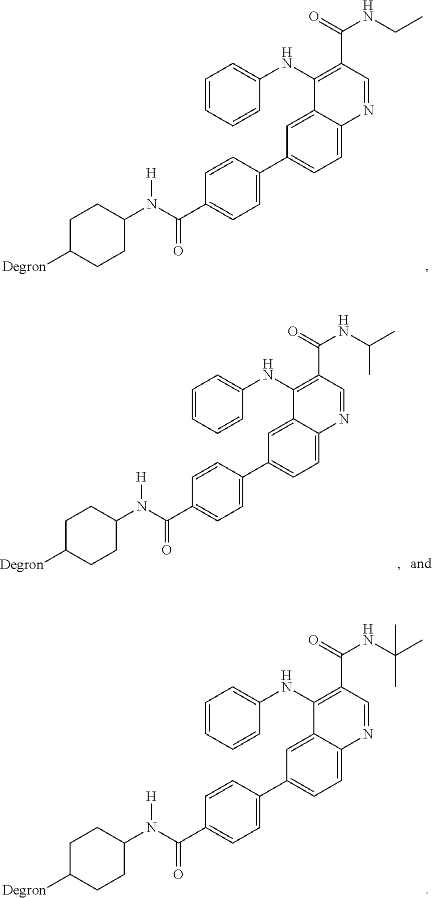

In some embodiments, a compound of Formula V is selected from:

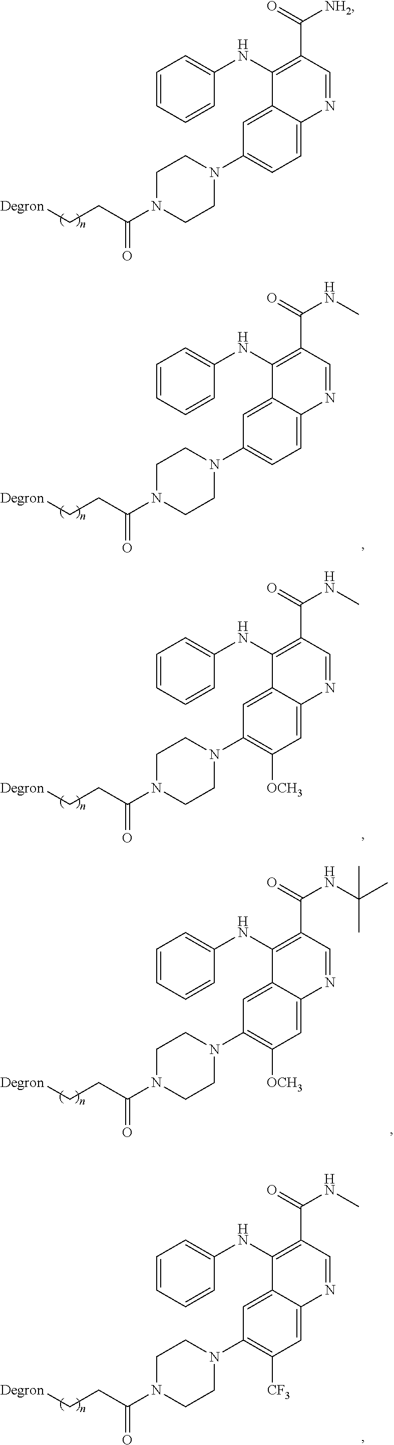

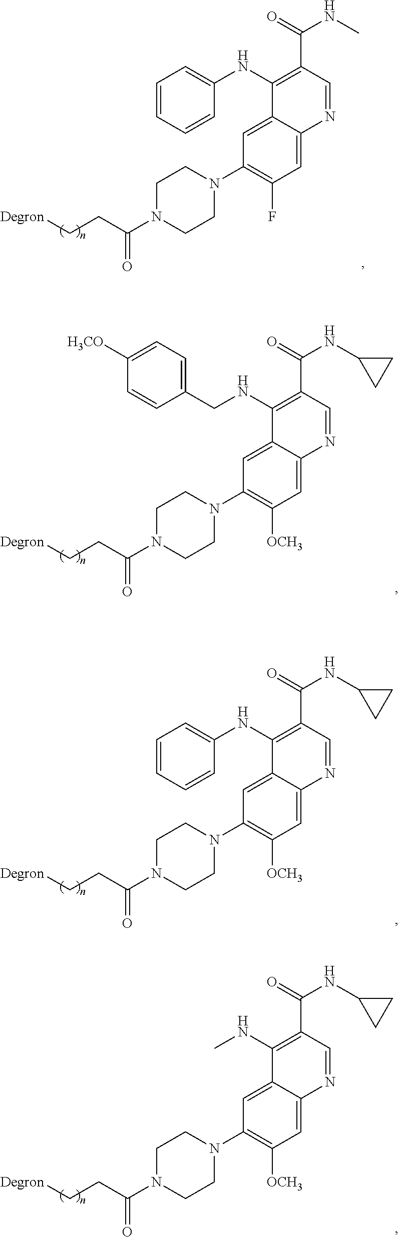

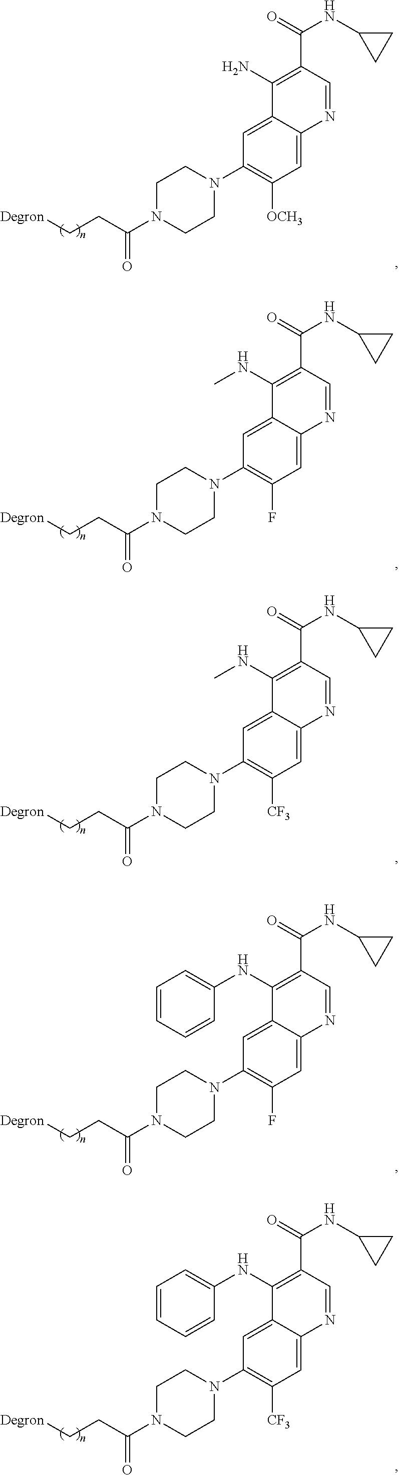

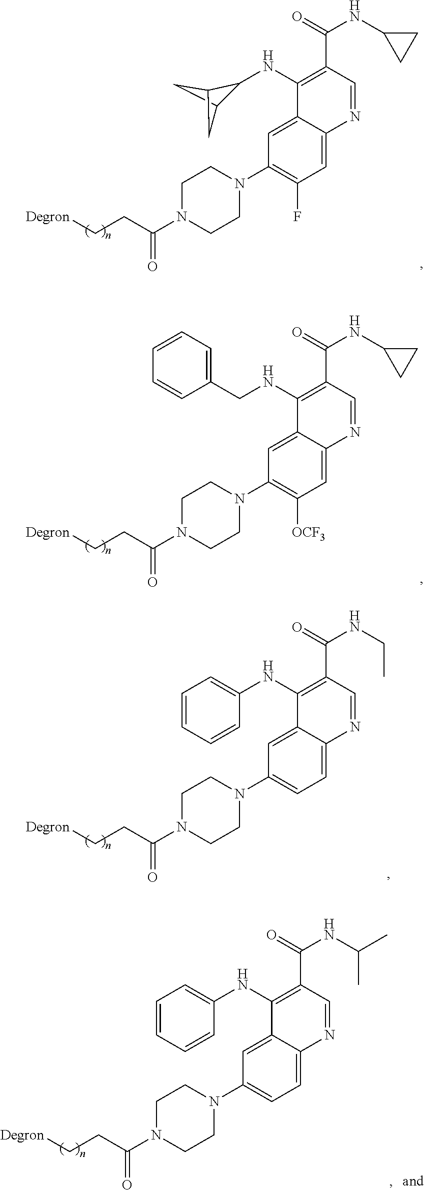

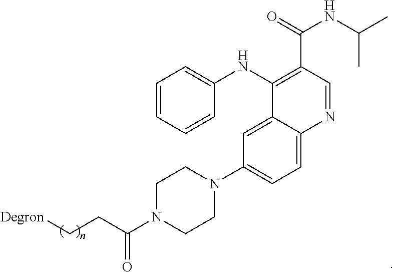

In some embodiments, a compound of Formula V is selected from:

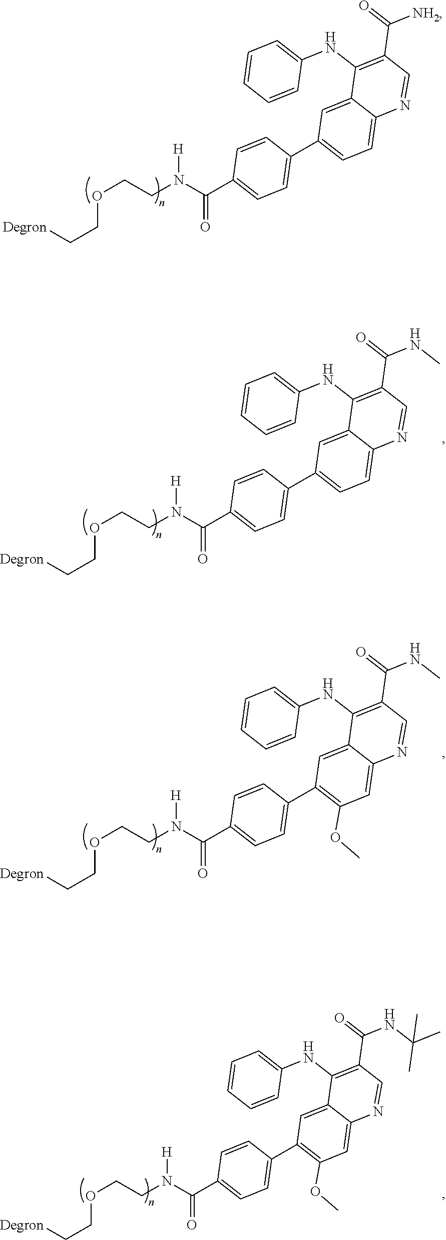

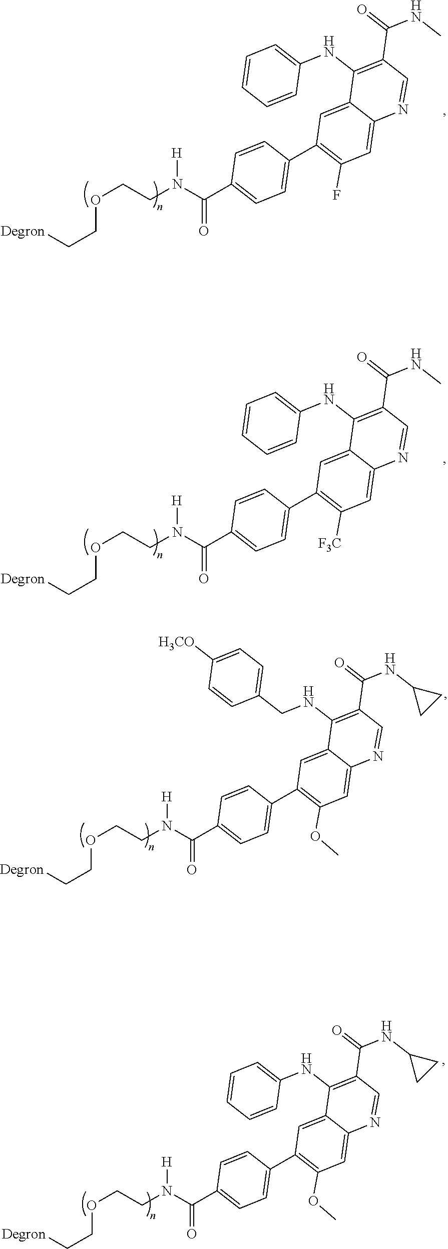

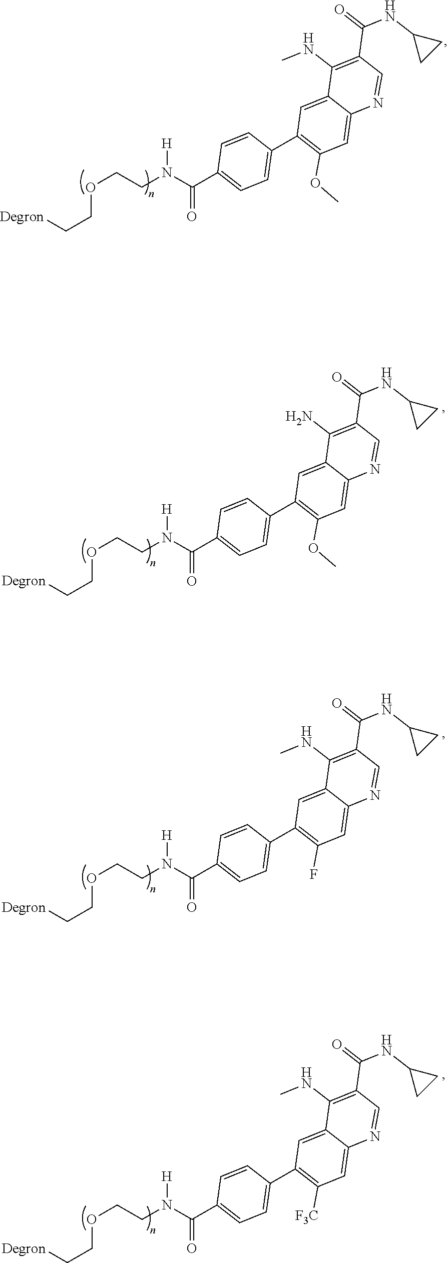

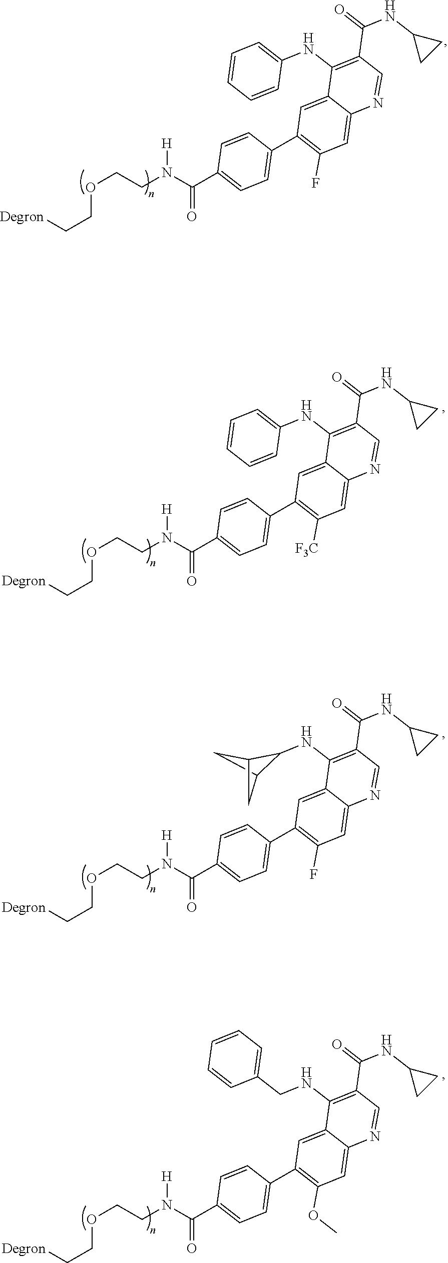

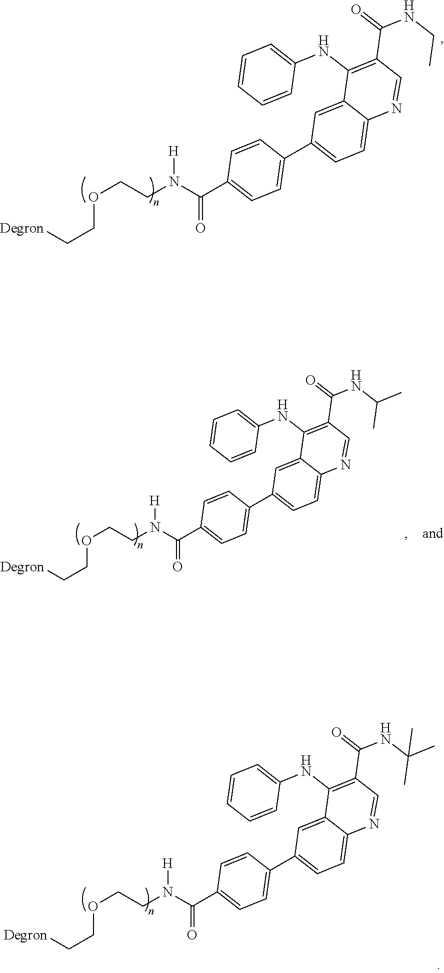

In some embodiments, a compound of Formula V is selected from:

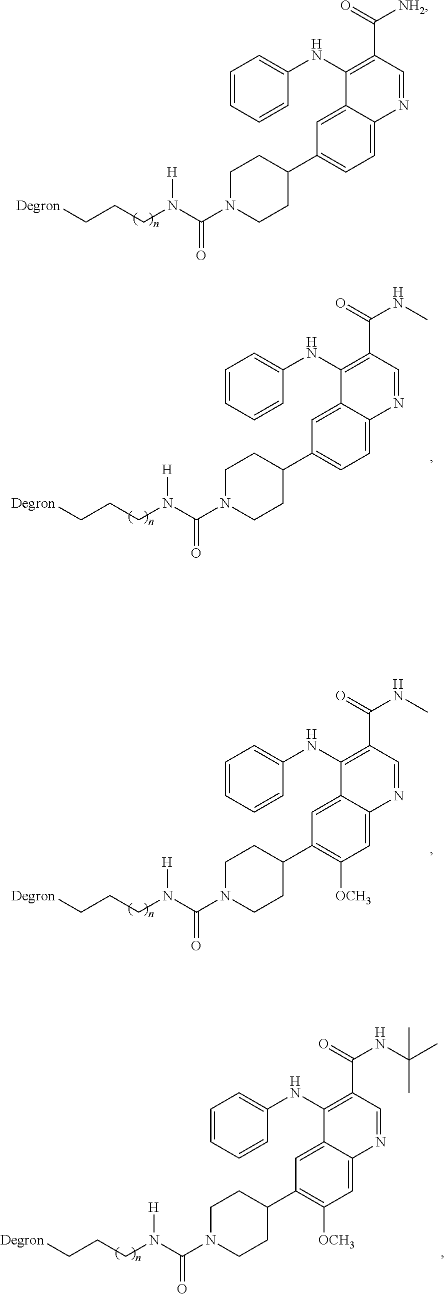

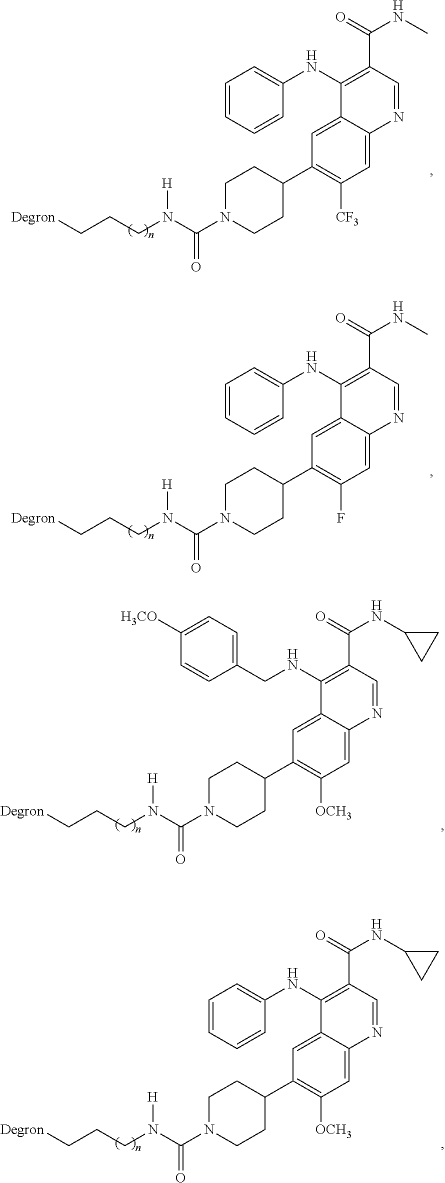

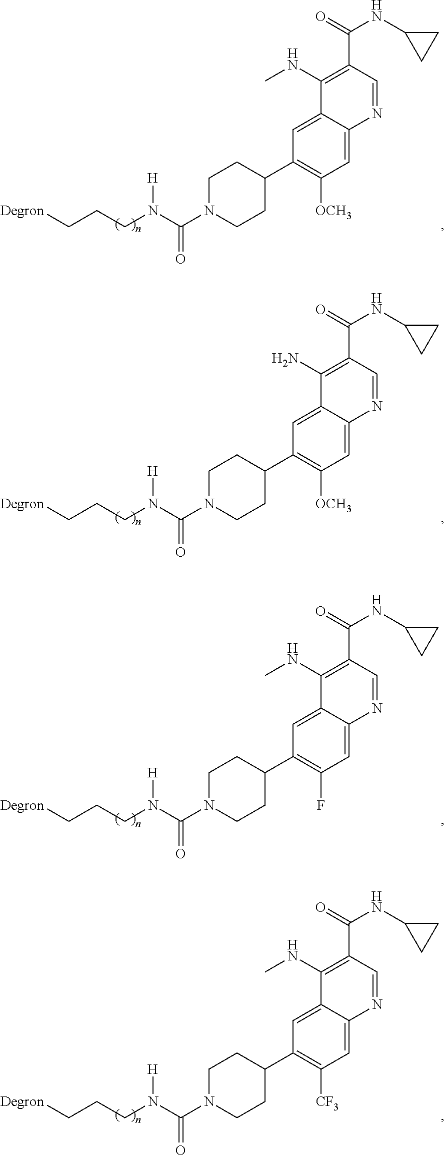

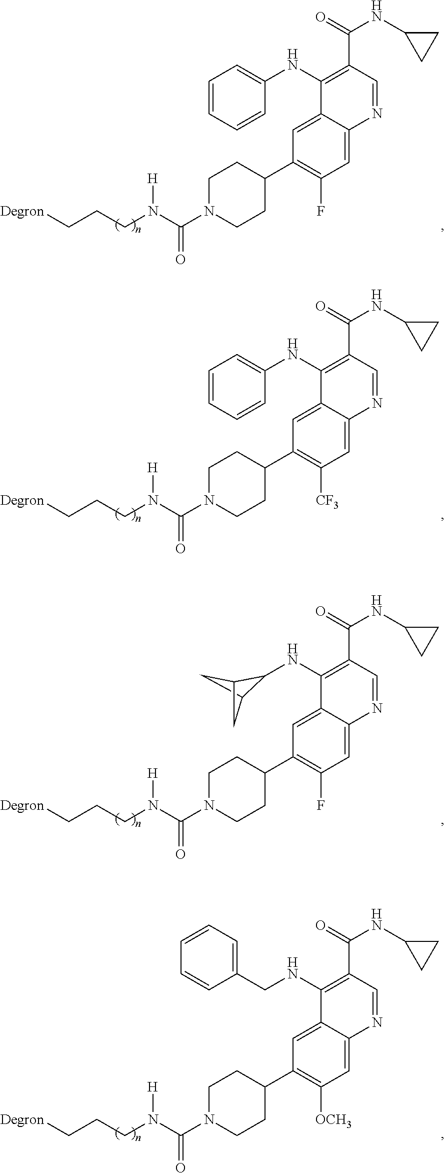

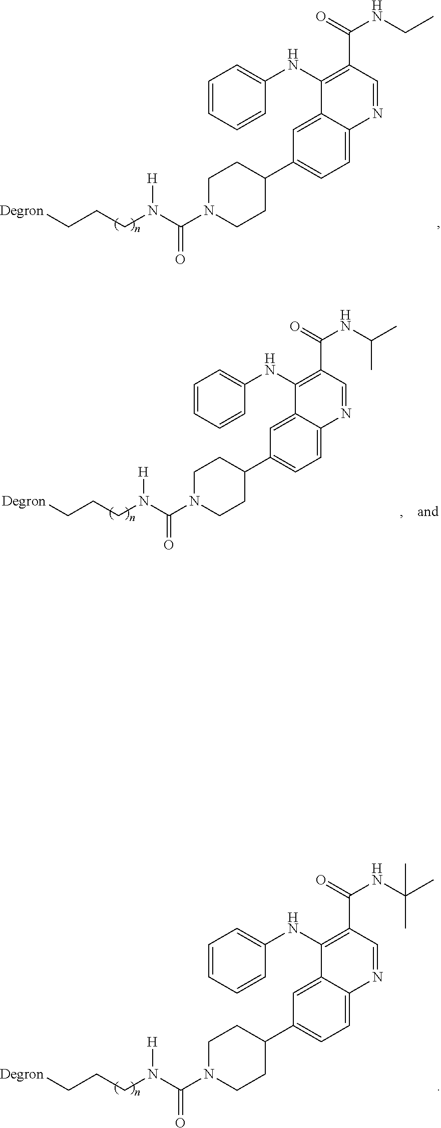

In some embodiments, a compound of Formula V is selected from:

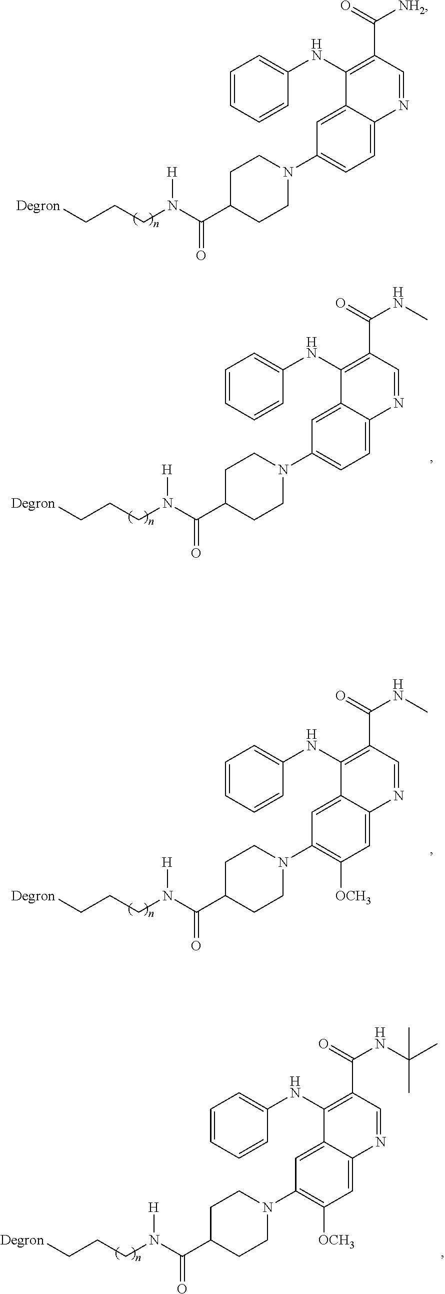

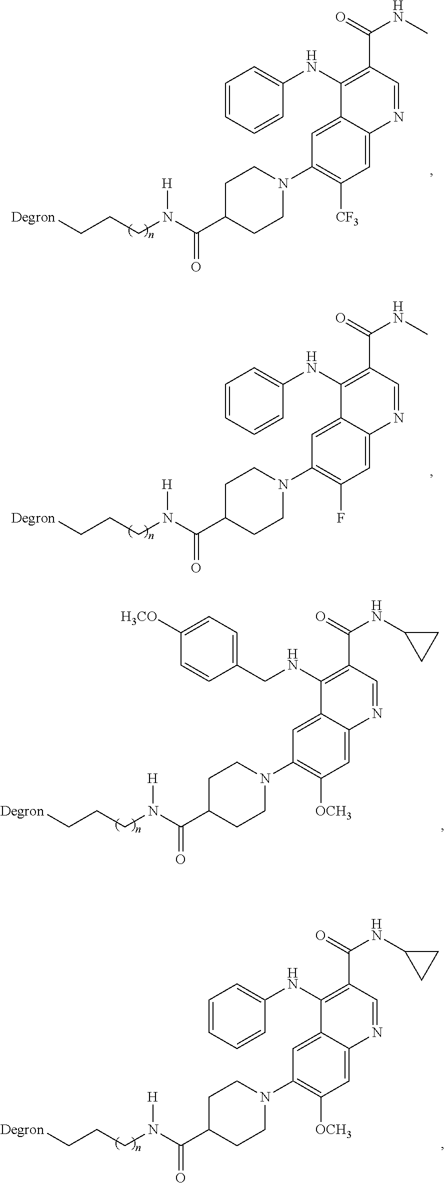

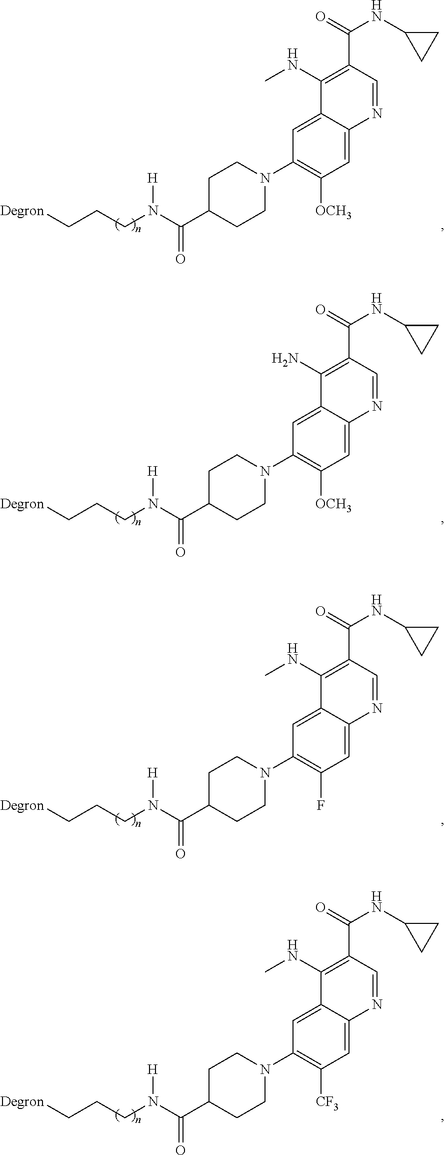

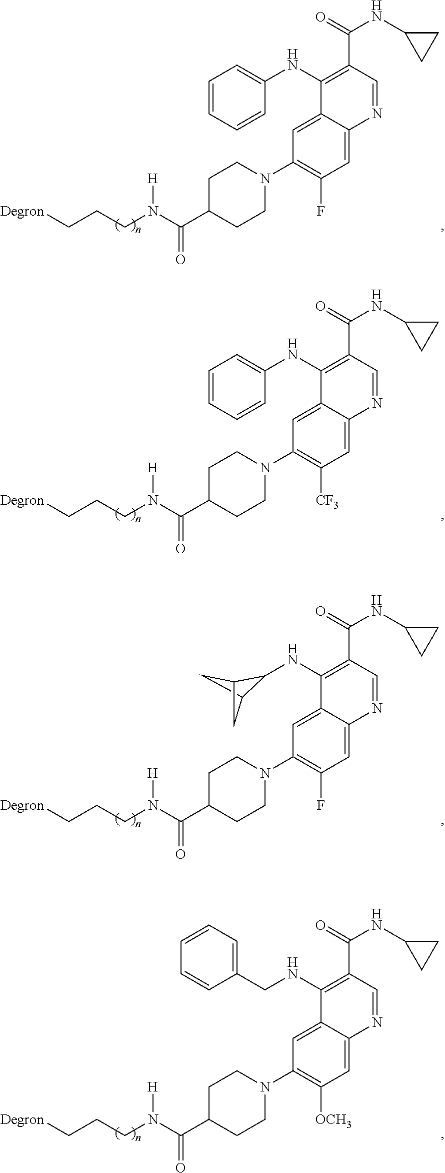

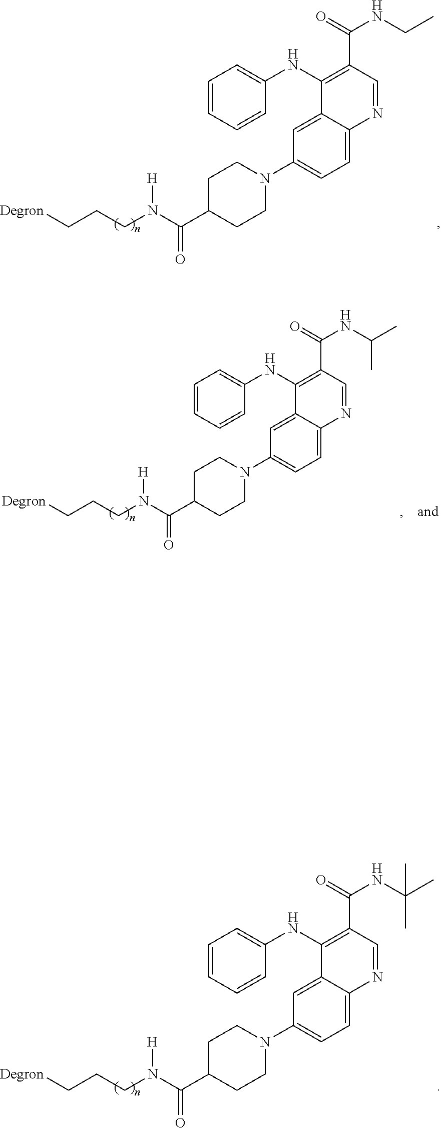

In some embodiments, a compound of Formula V is selected from:

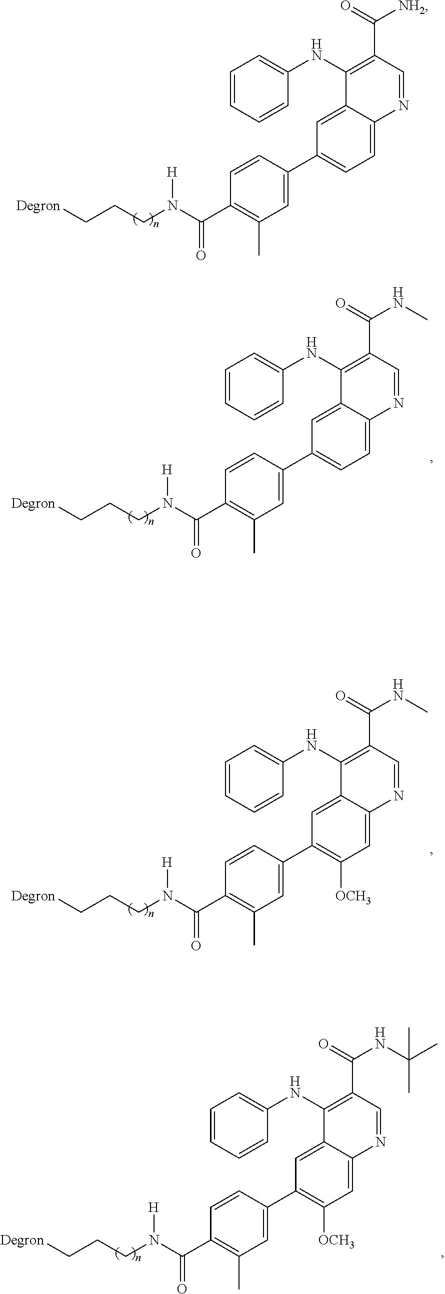

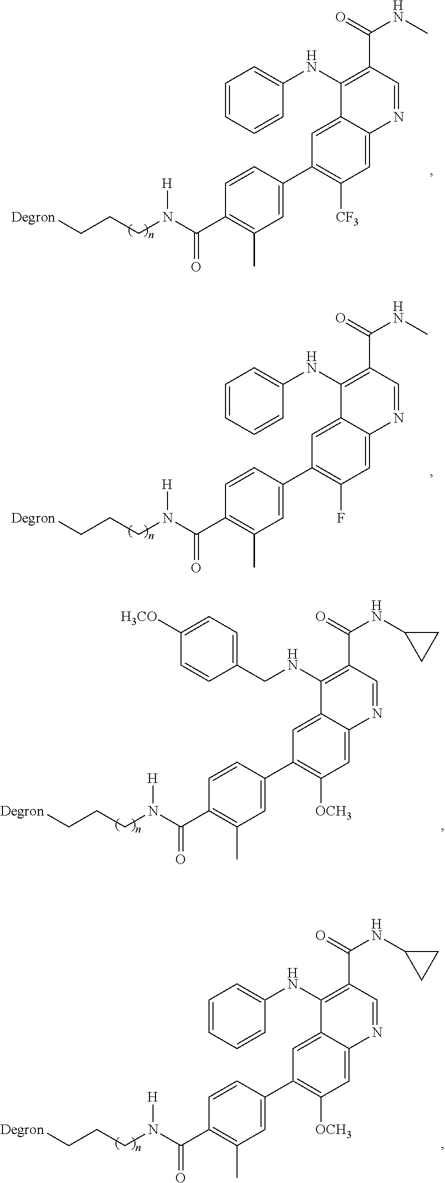

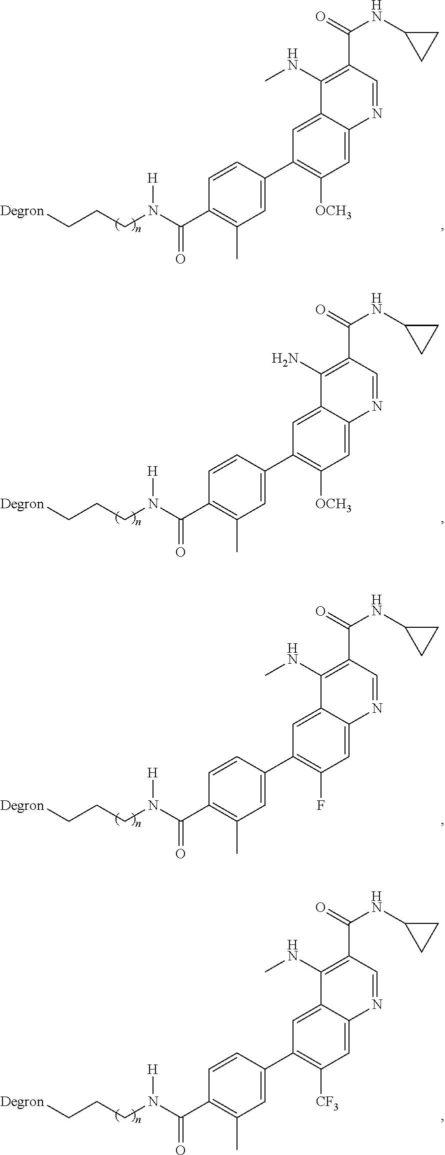

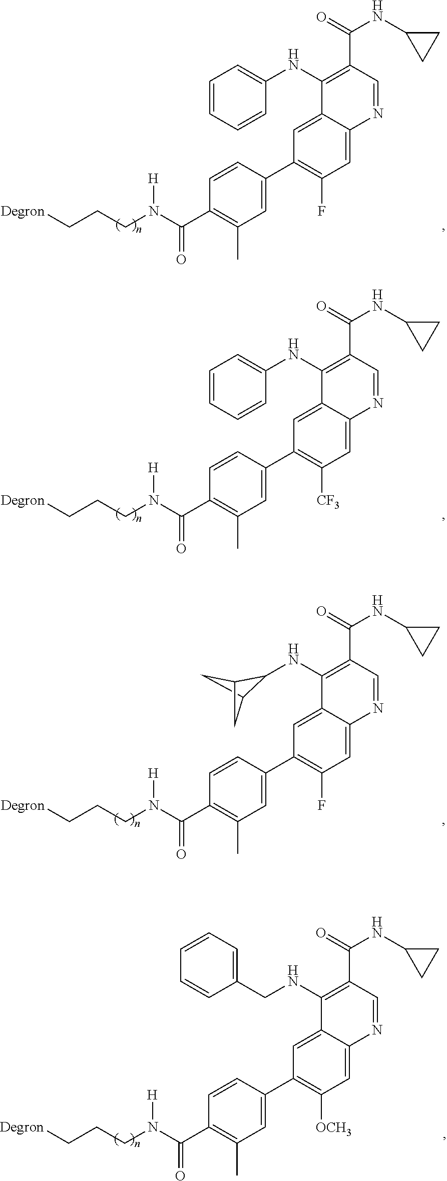

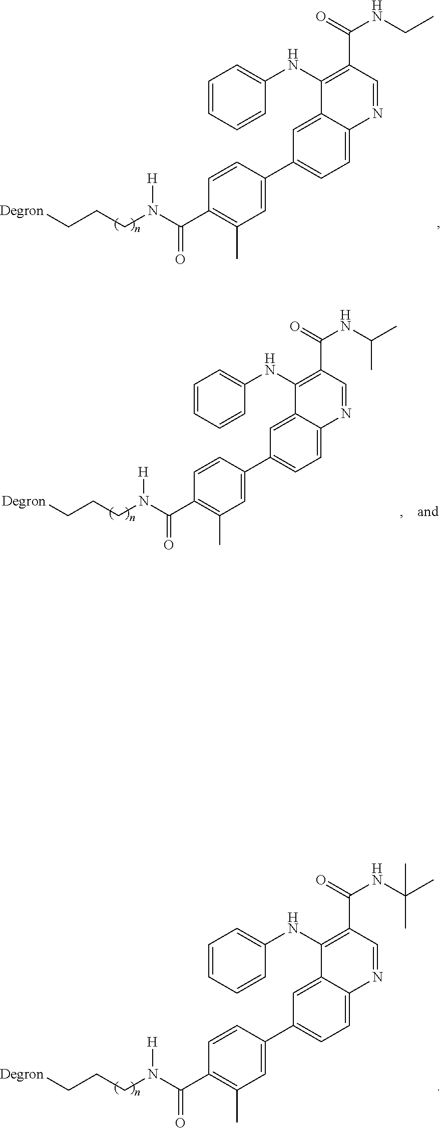

In some embodiments, the compound of Formula V is selected from:

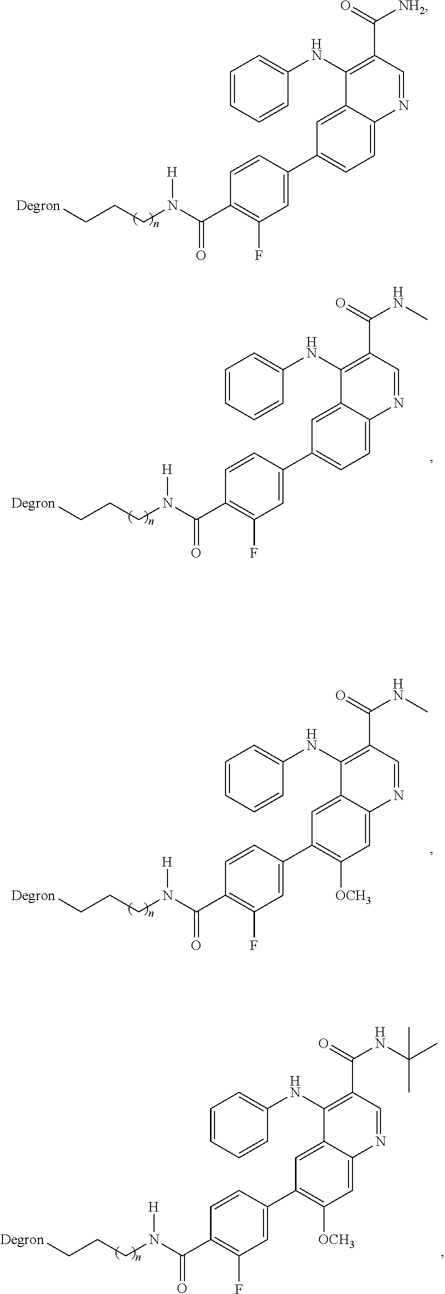

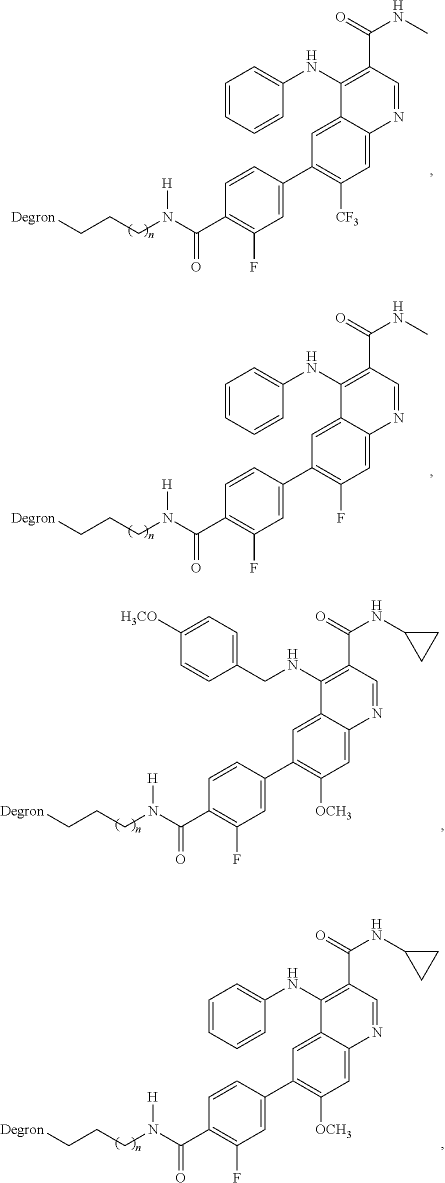

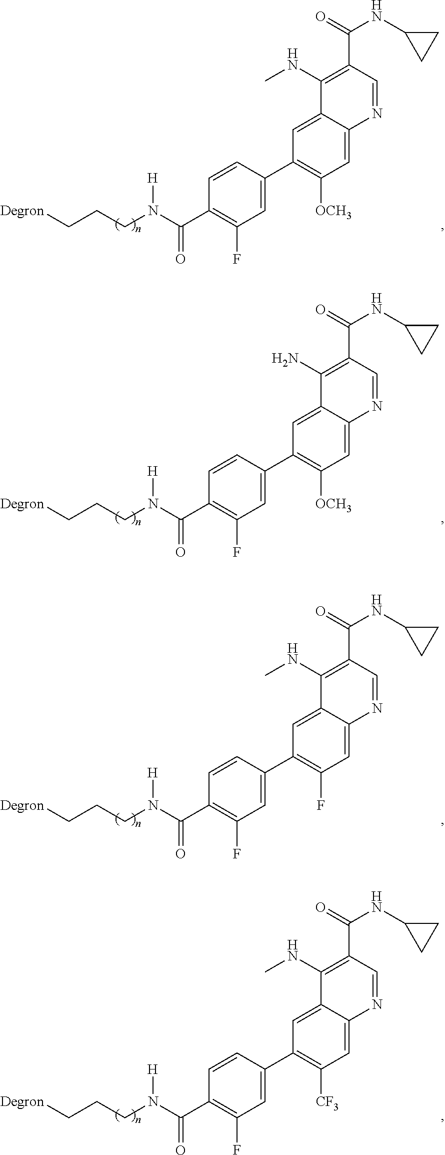

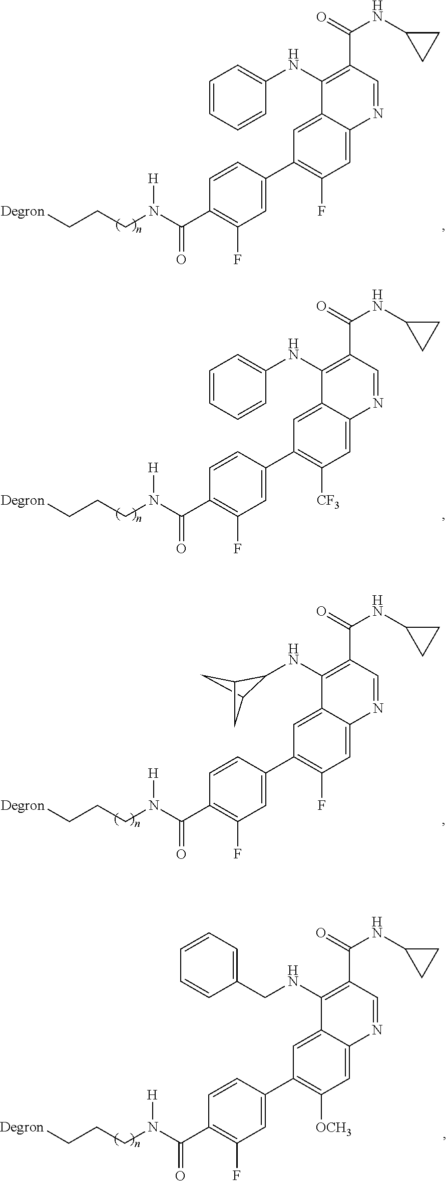

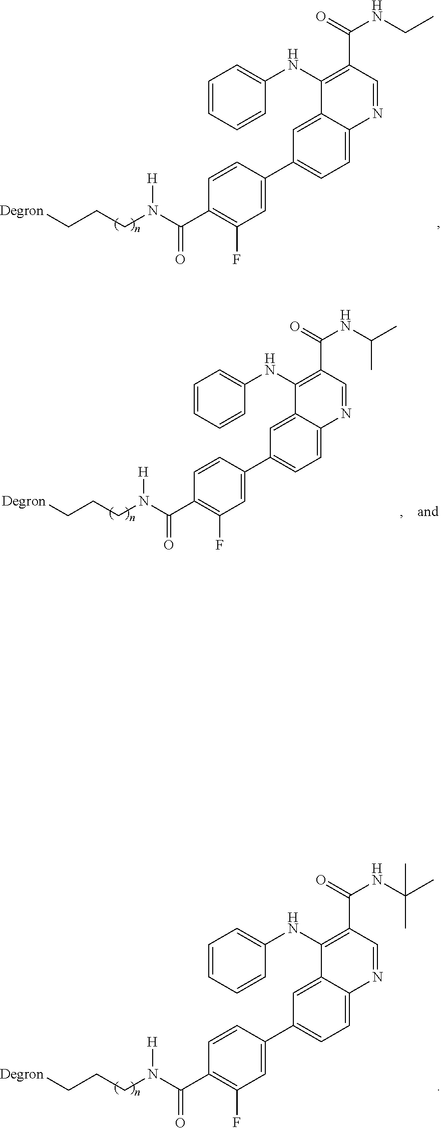

In some embodiments, the compound of Formula V is selected from:

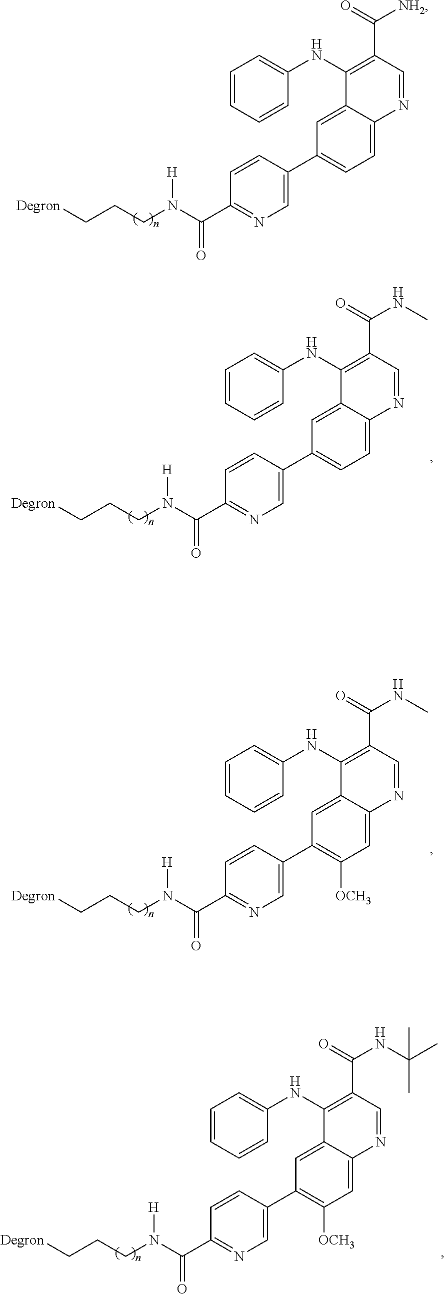

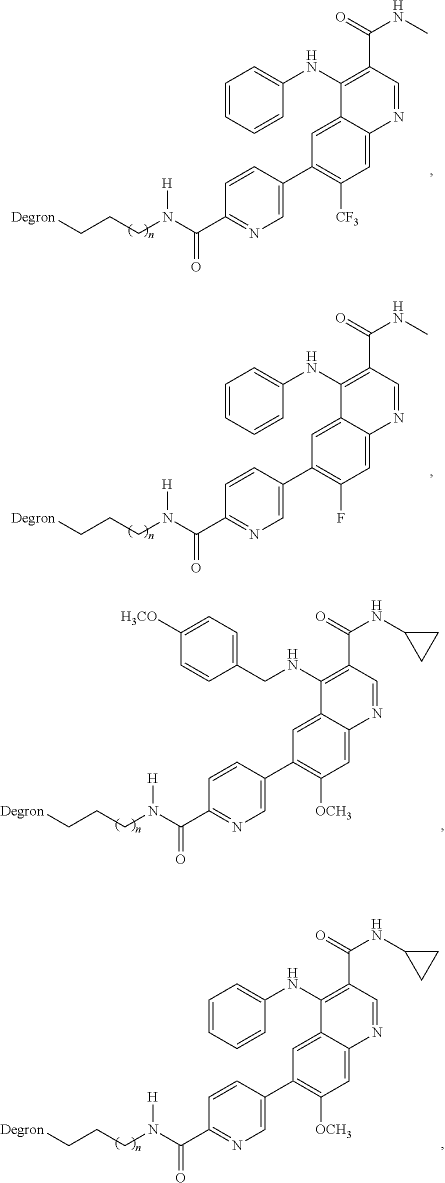

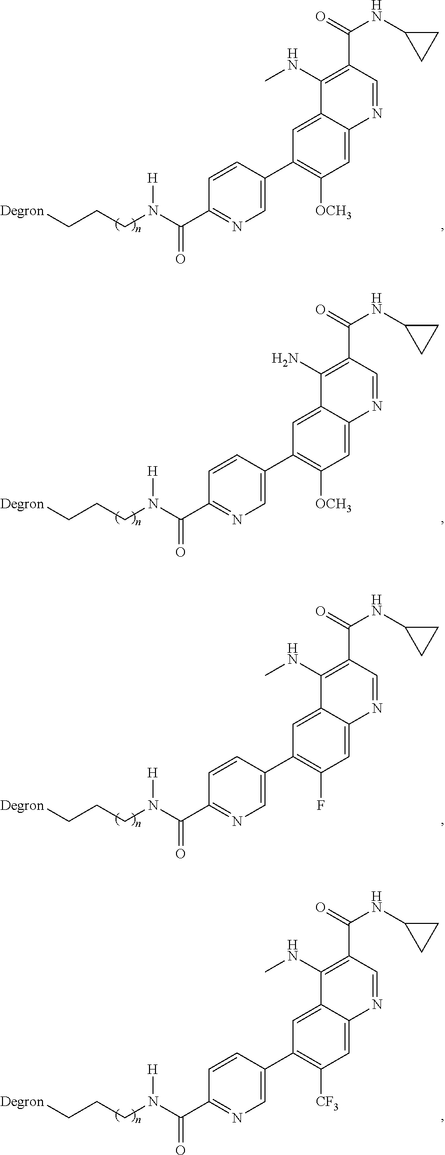

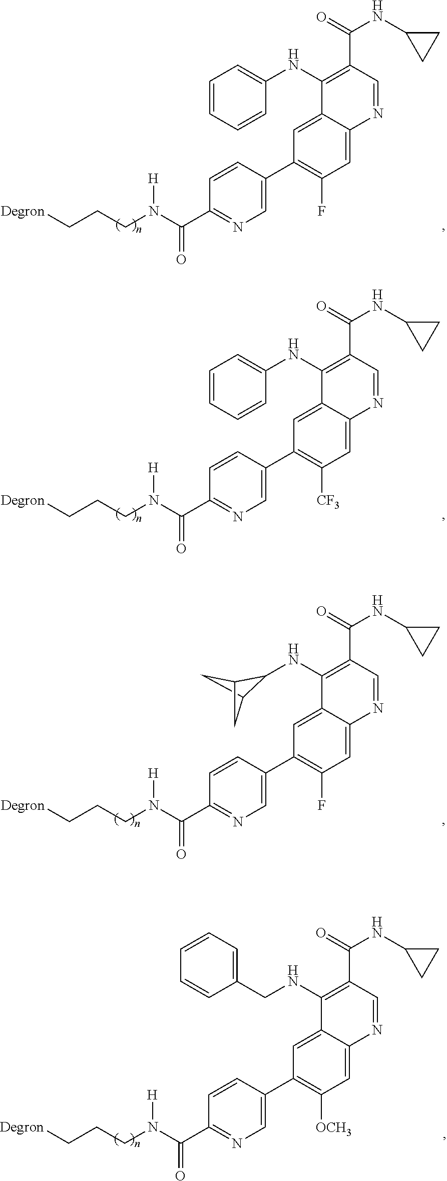

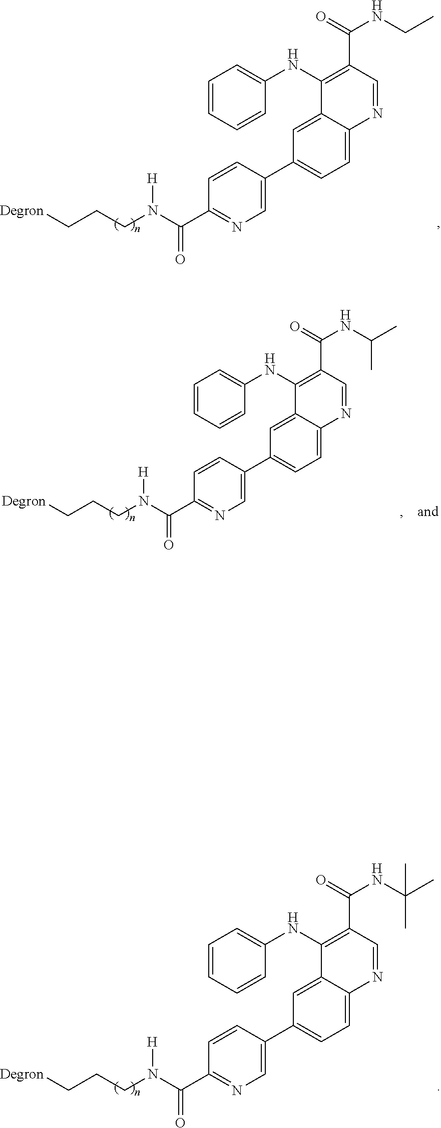

In some embodiments, the compound of Formula V is selected from:

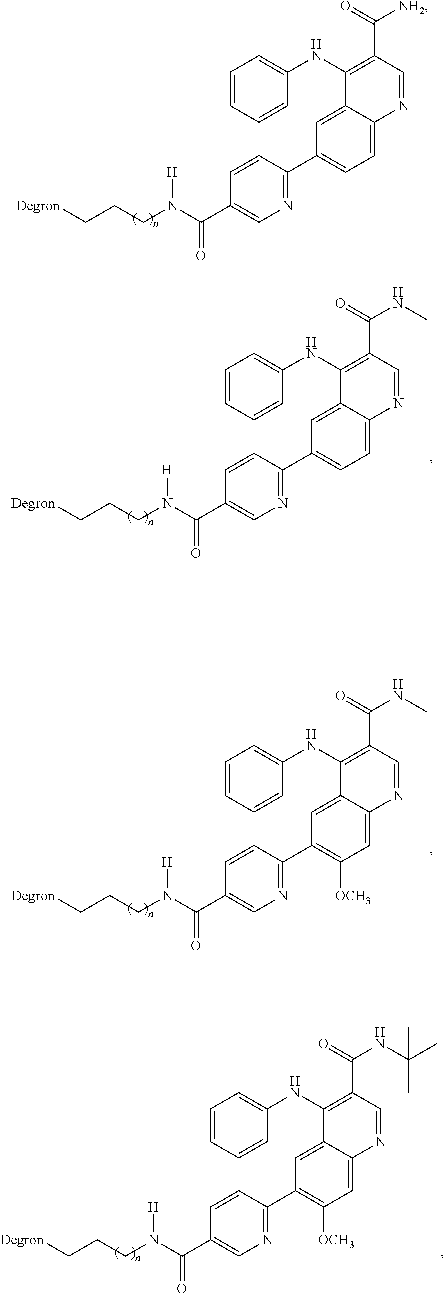

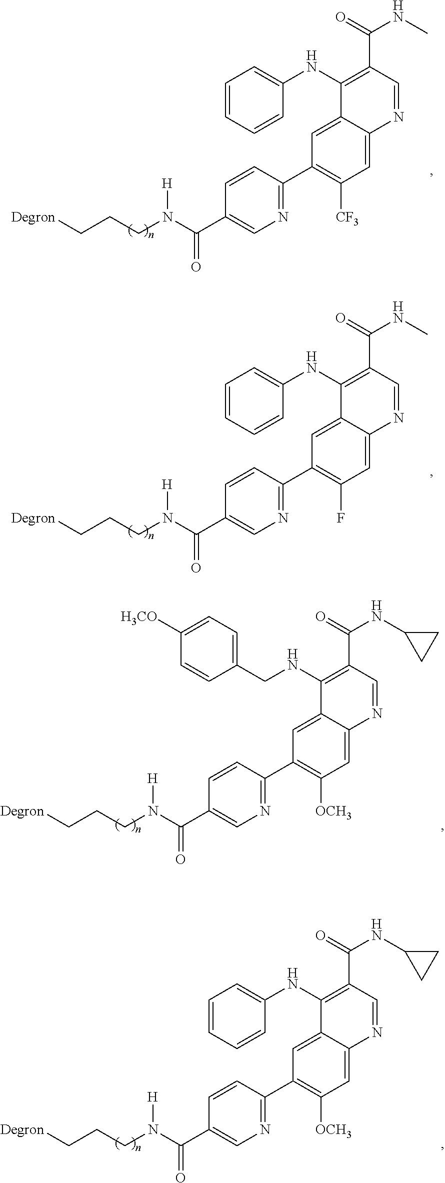

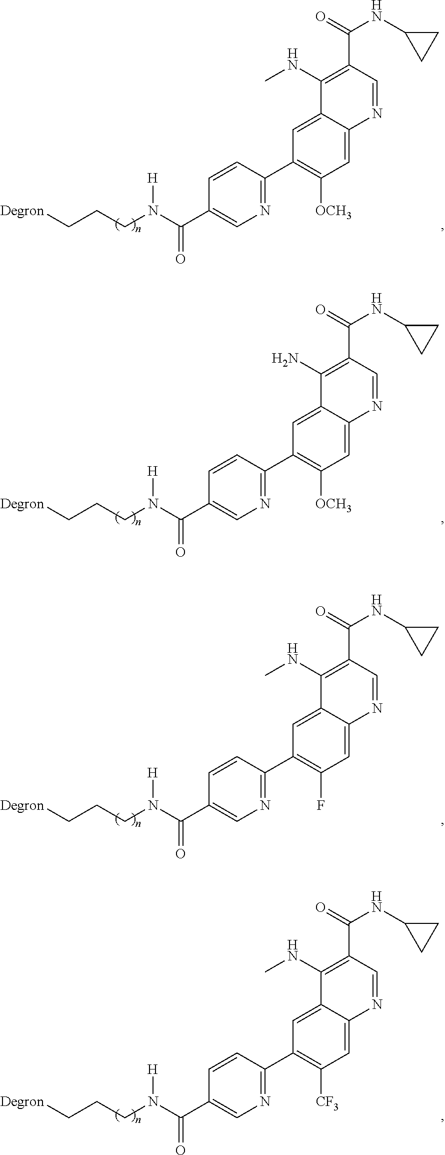

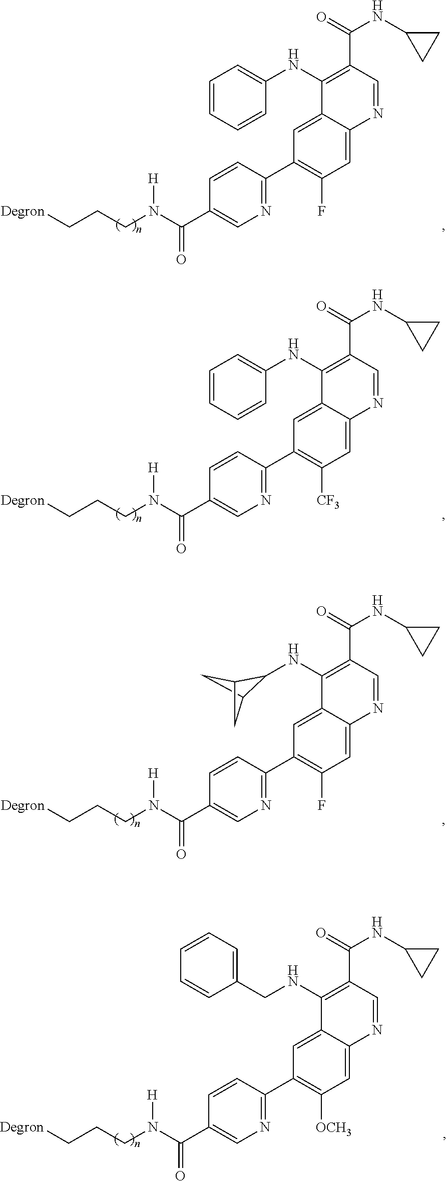

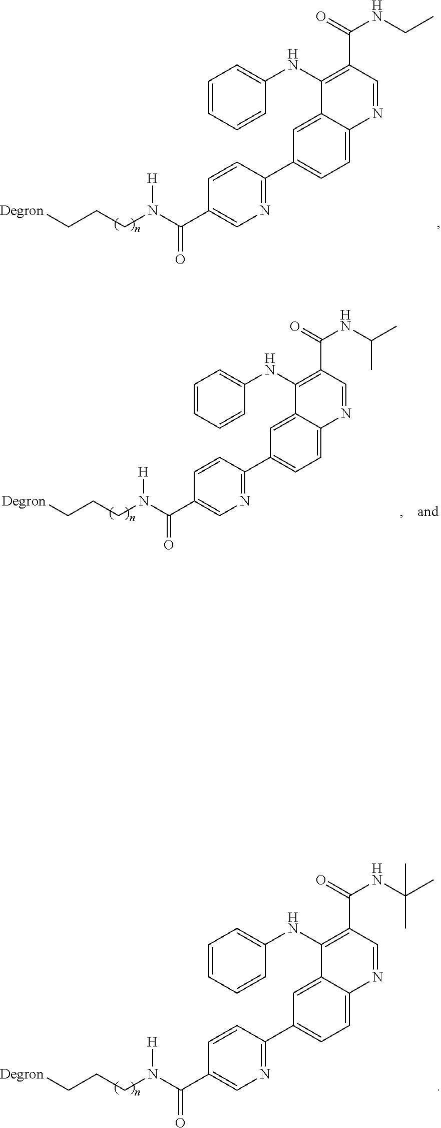

In some embodiments, the compound of Formula V is selected from:

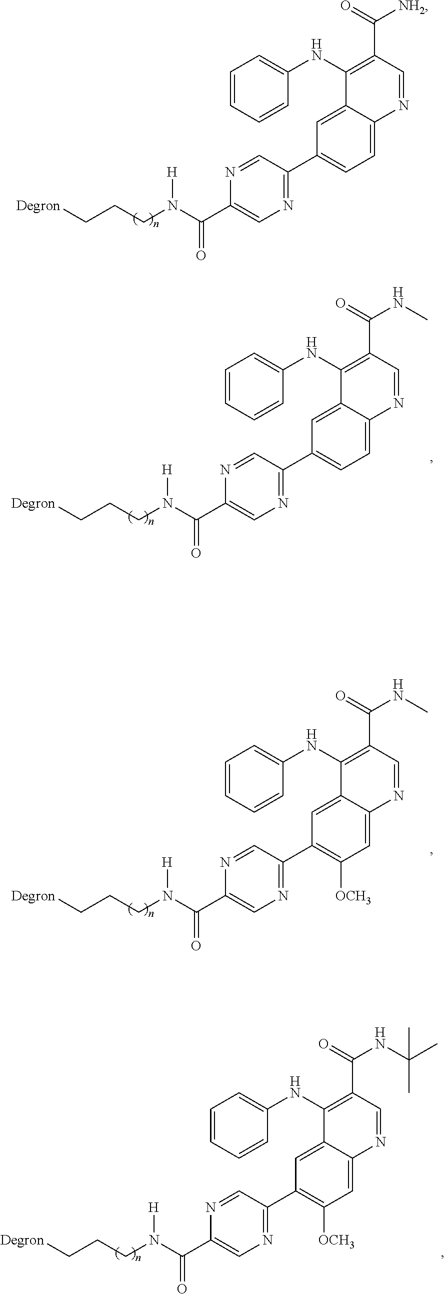

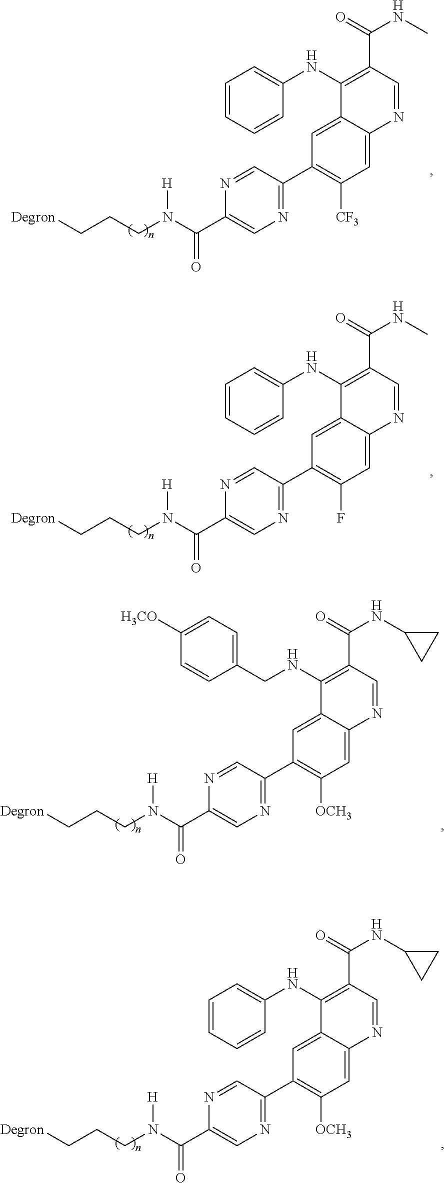

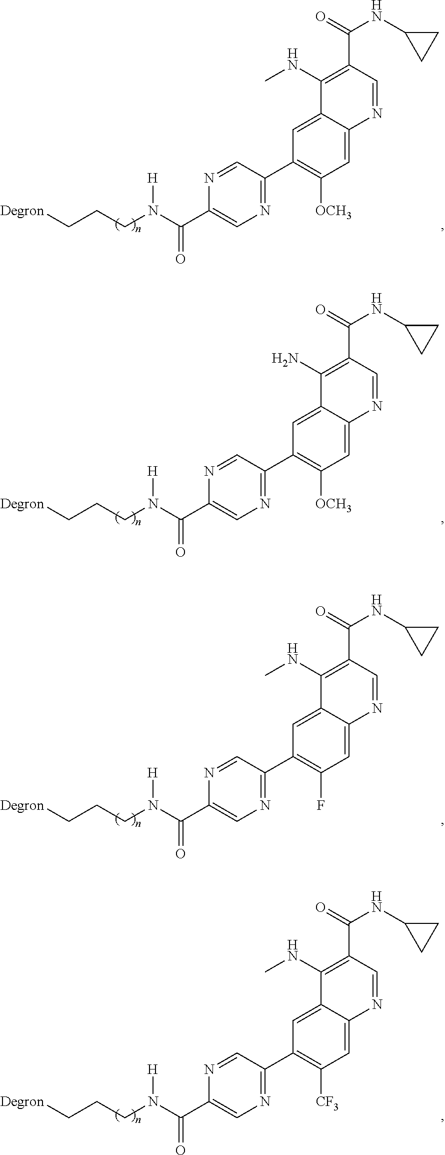

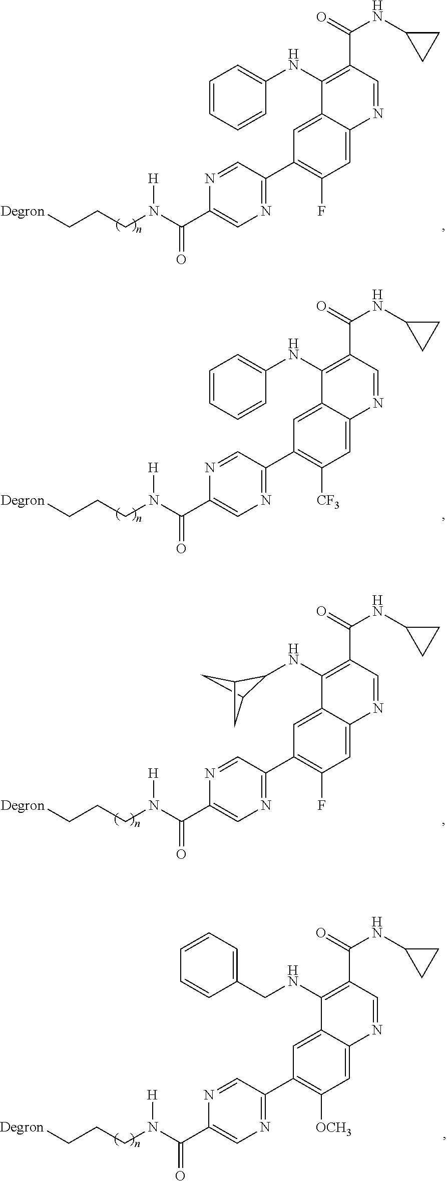

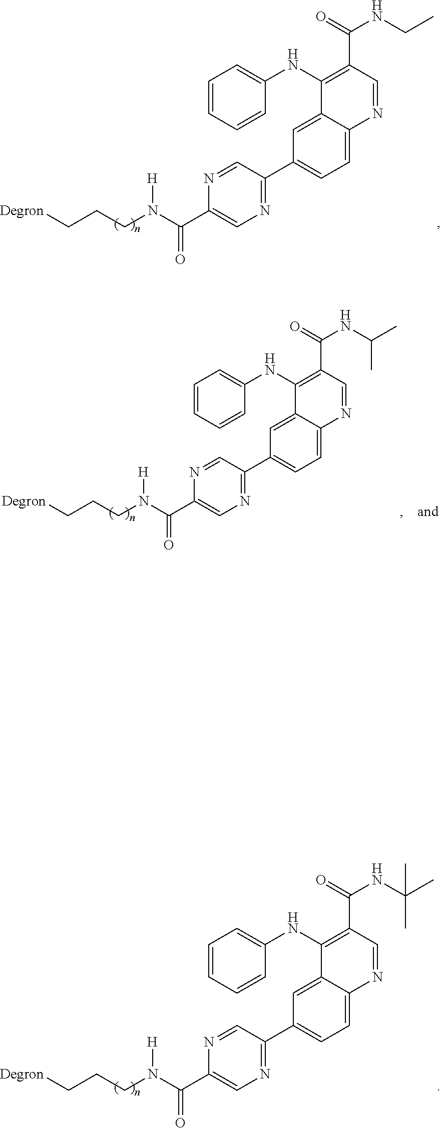

In some embodiments, the compound of Formula V is selected from:

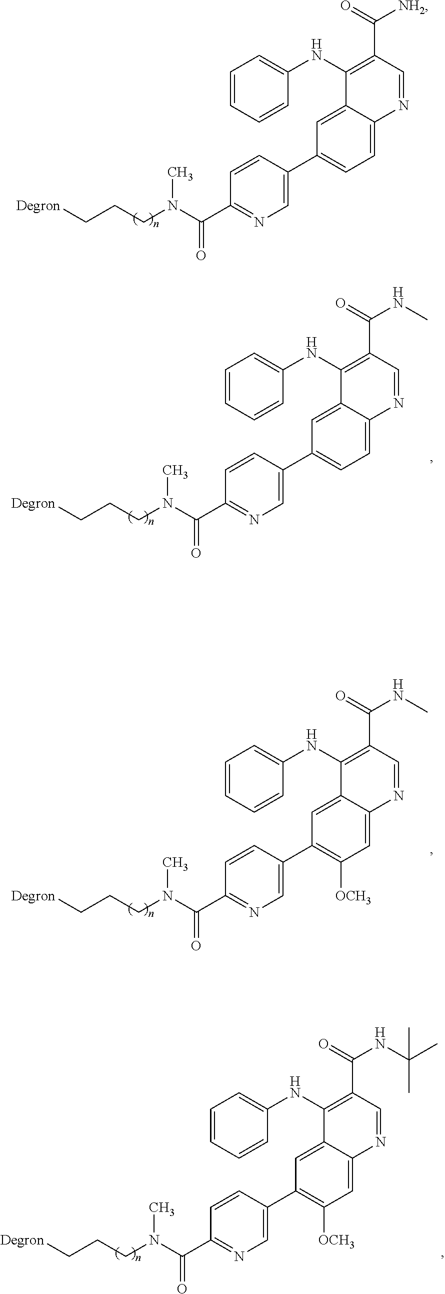

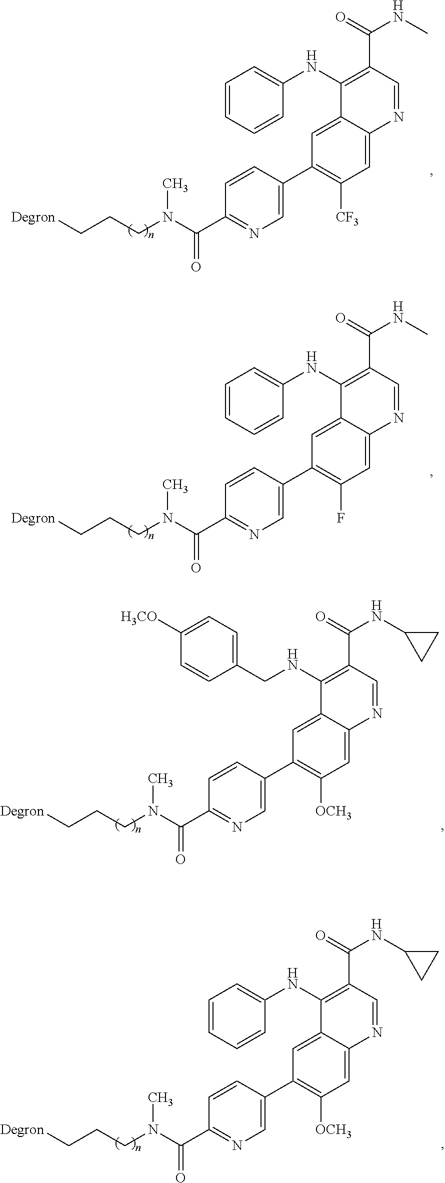

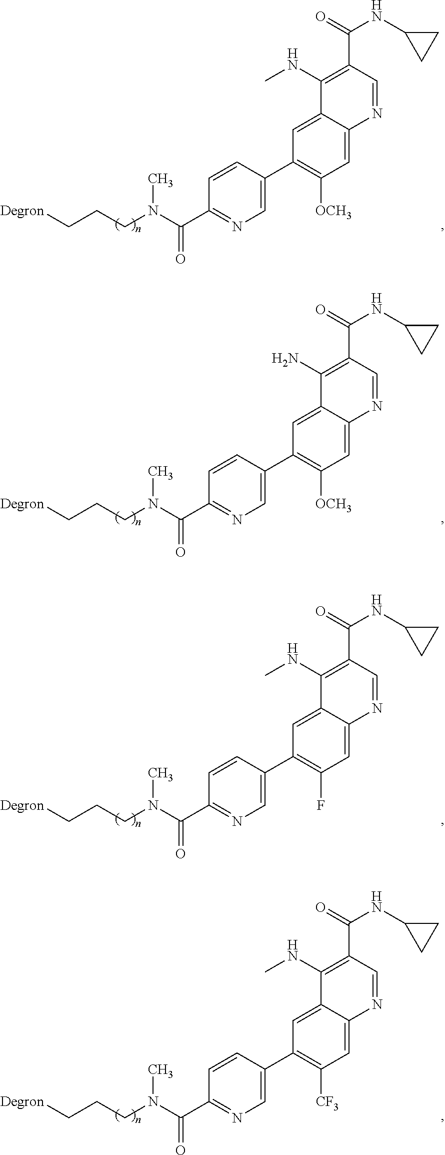

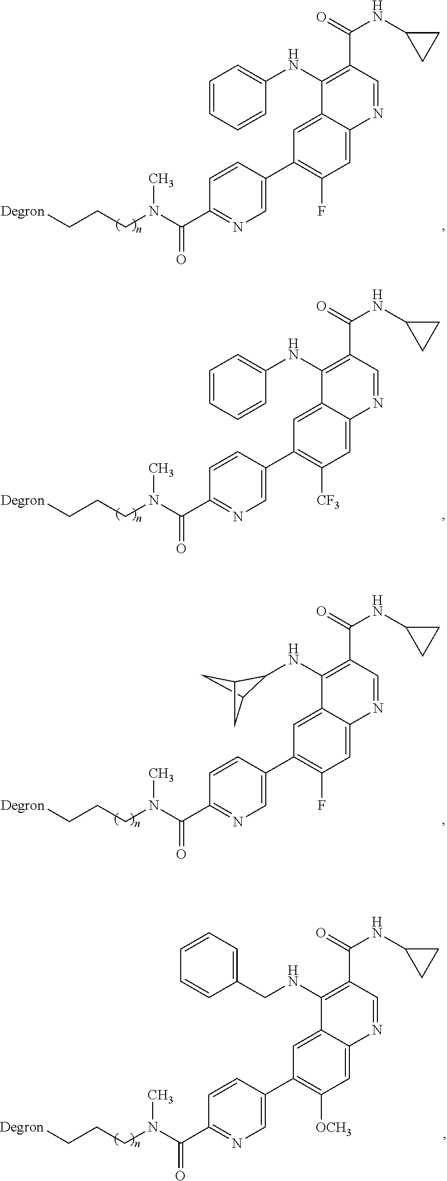

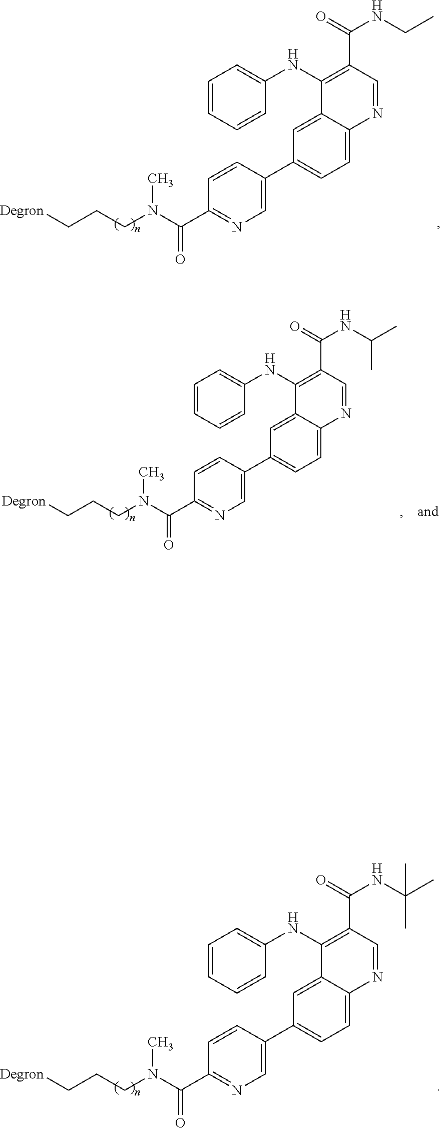

In some embodiments, the compound of Formula V is selected from:

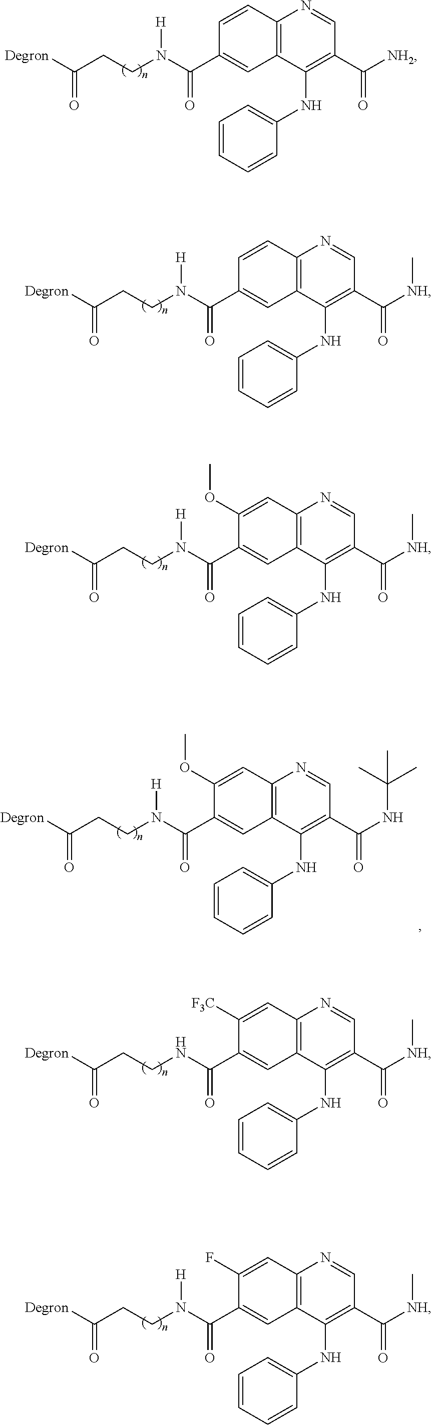

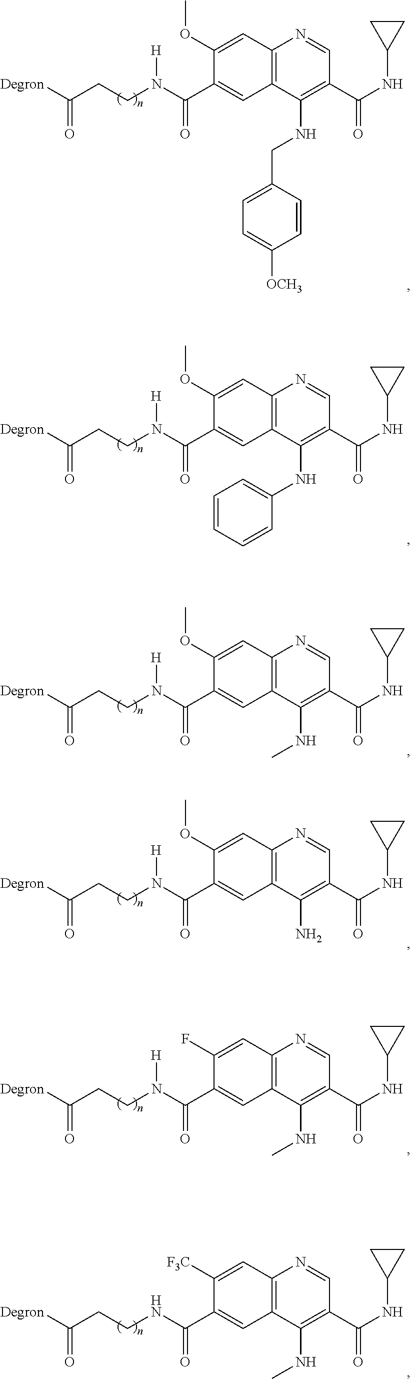

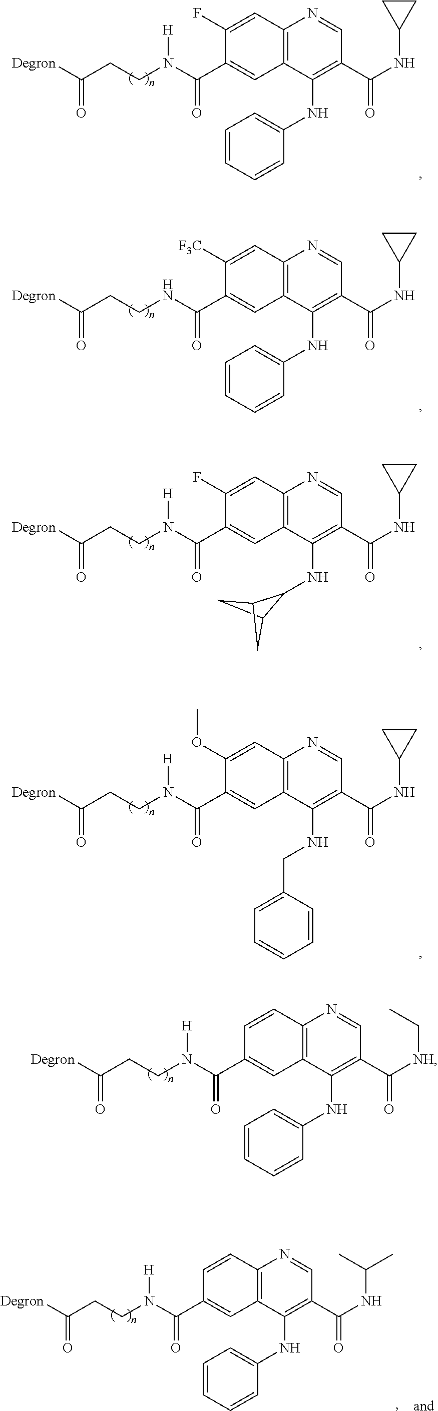

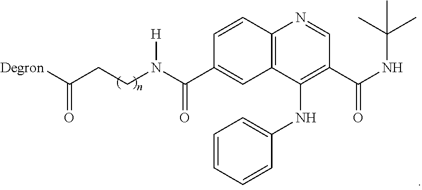

In some embodiments, the compound of Formula V is selected from:

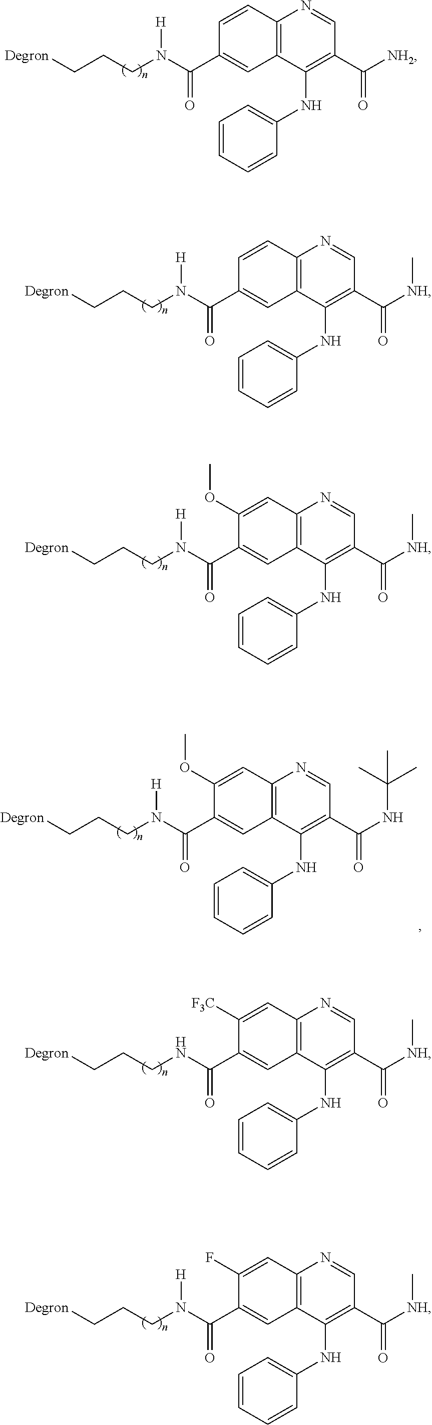

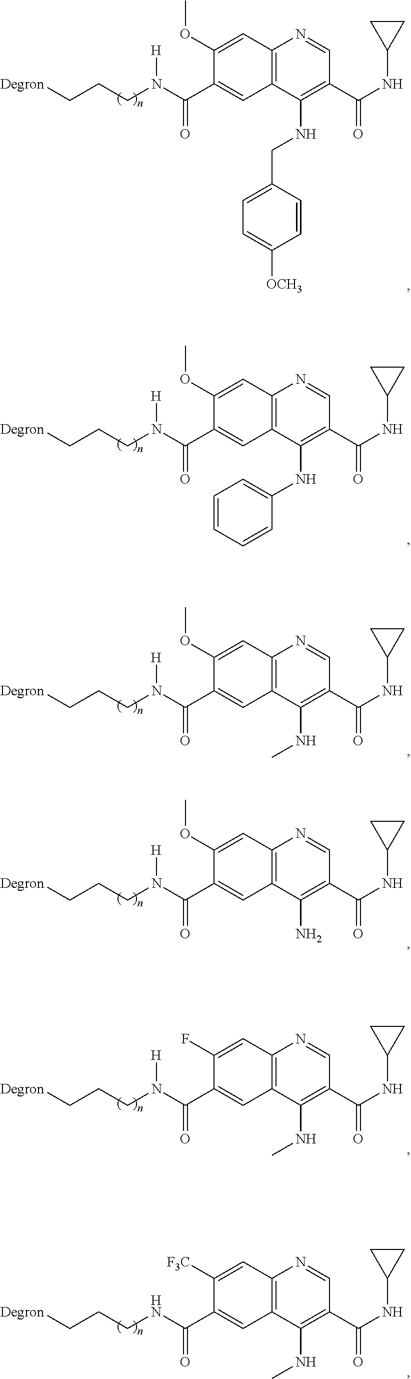

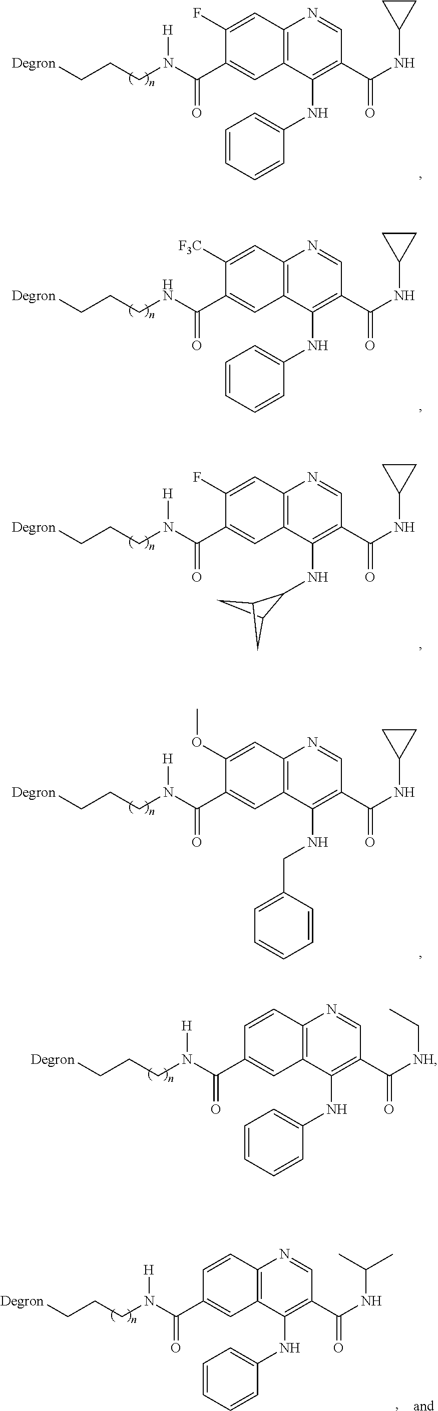

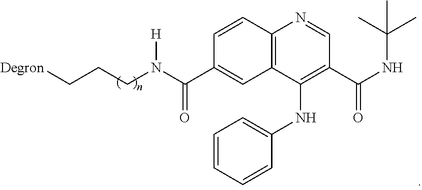

In some embodiments, the compound of Formula V is selected from:

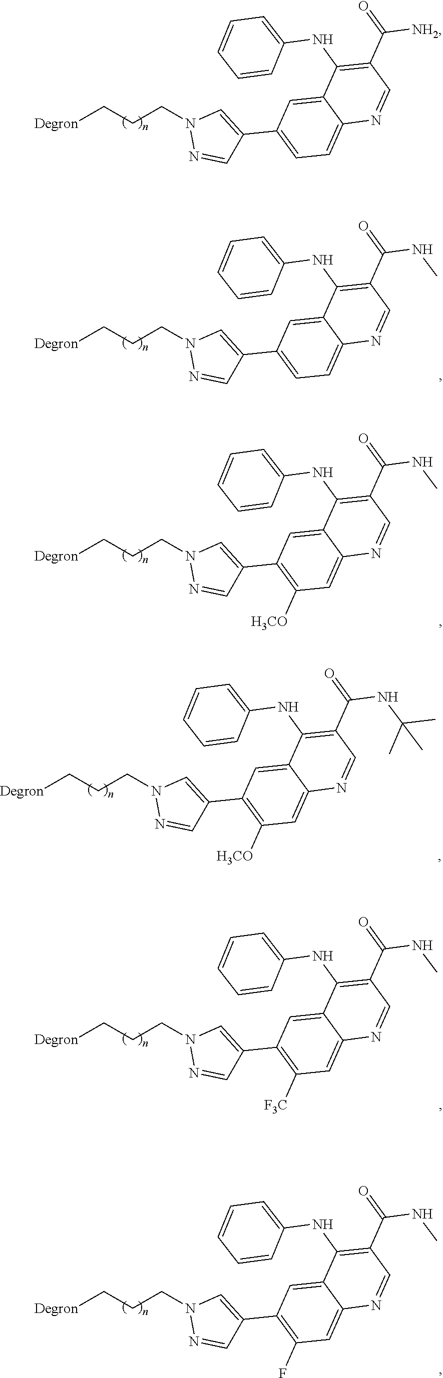

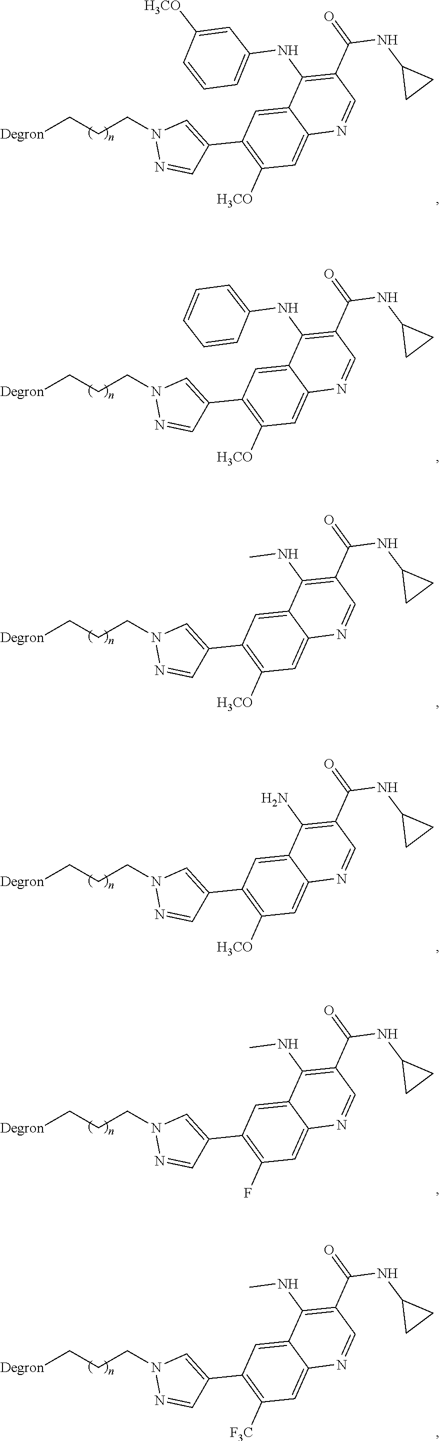

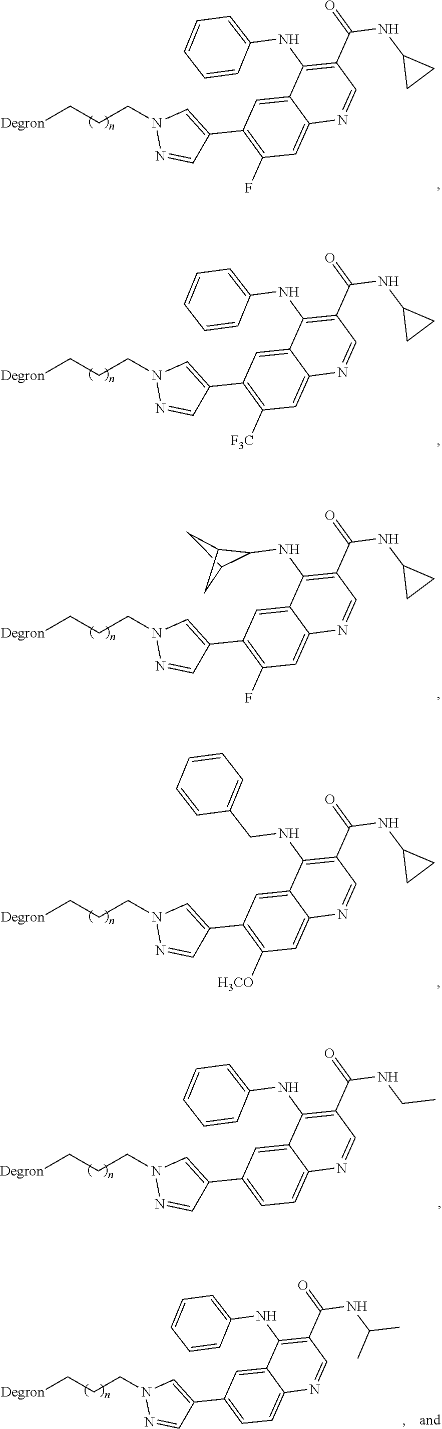

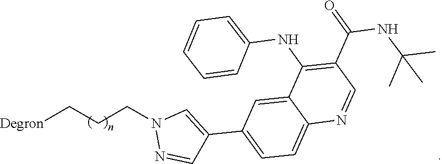

In some embodiments, the compound of Formula V is selected from:

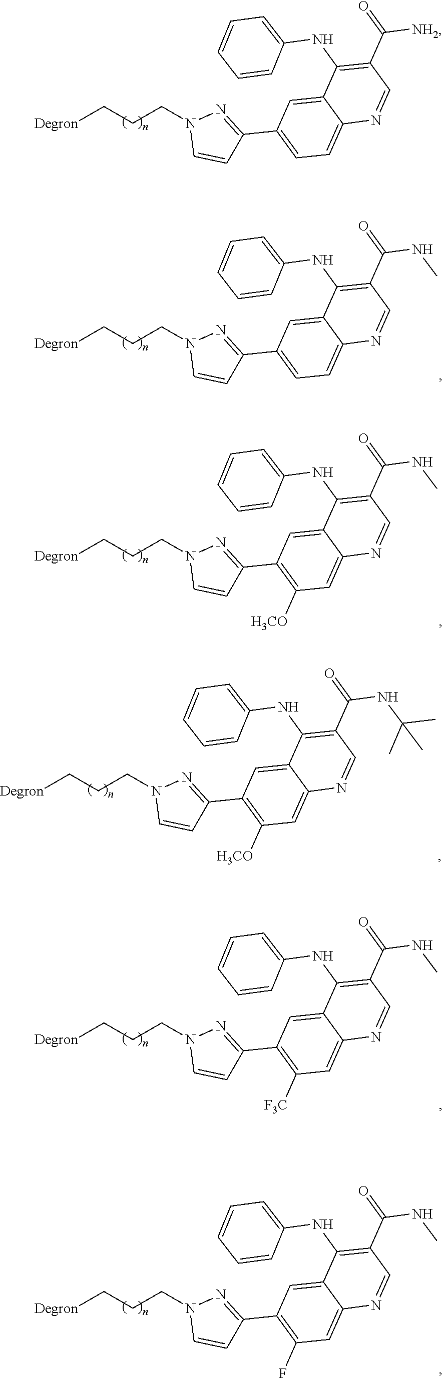

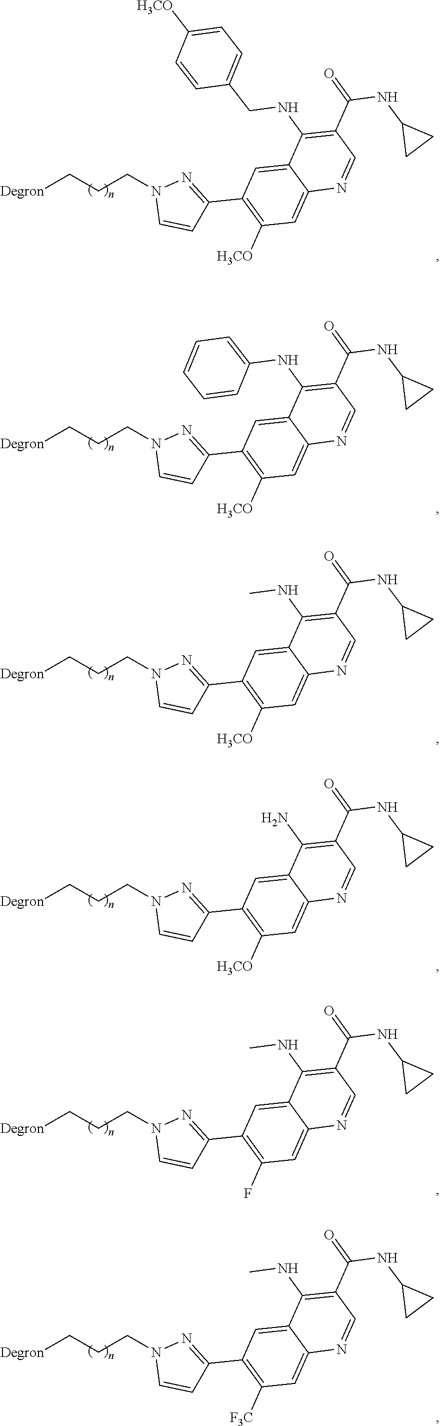

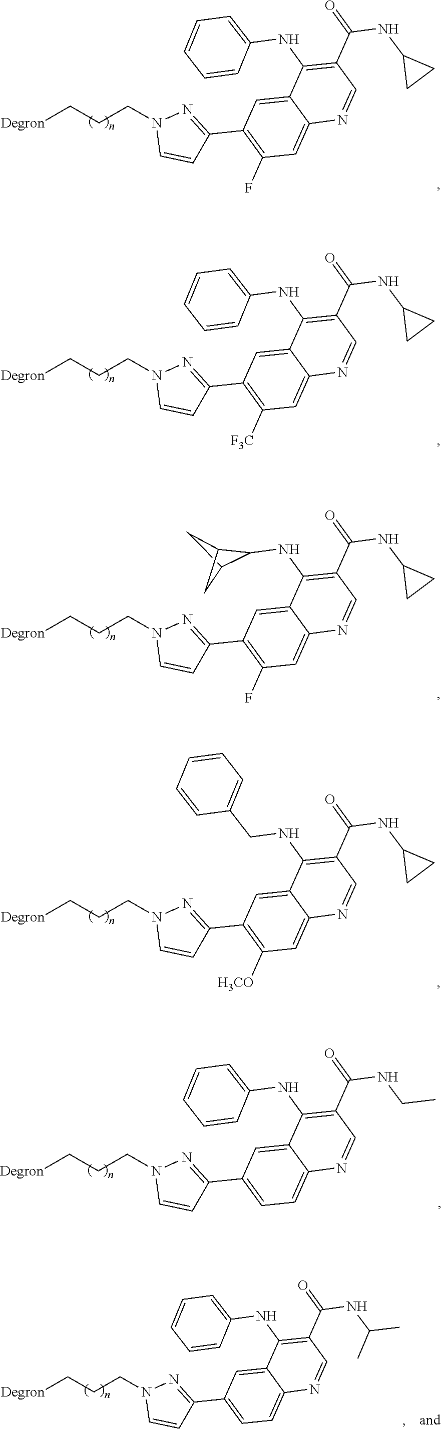

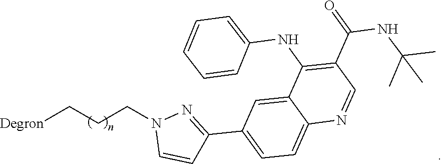

In some embodiments, the compound of Formula V is selected from:

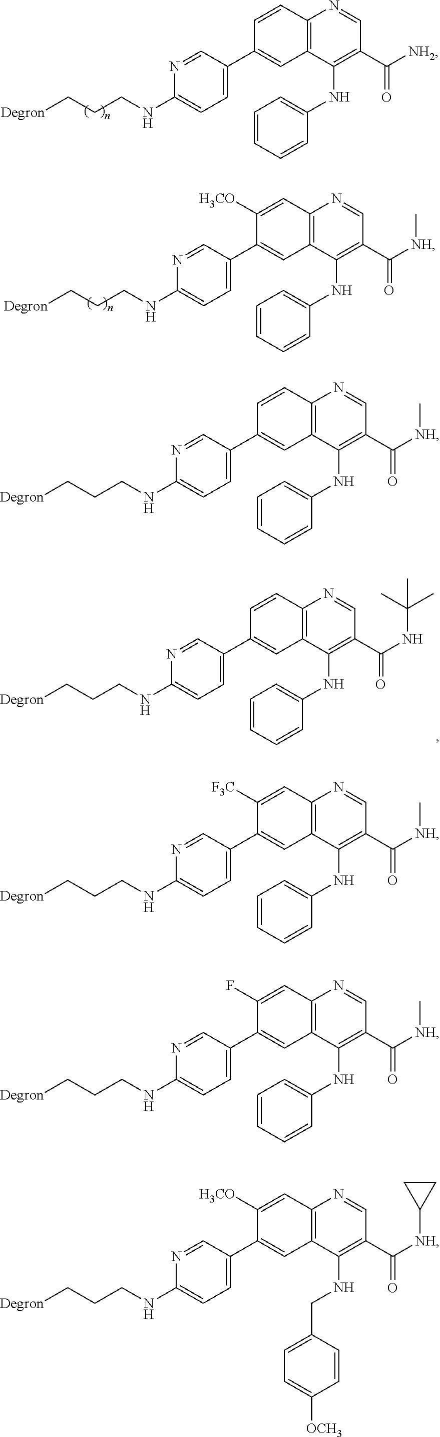

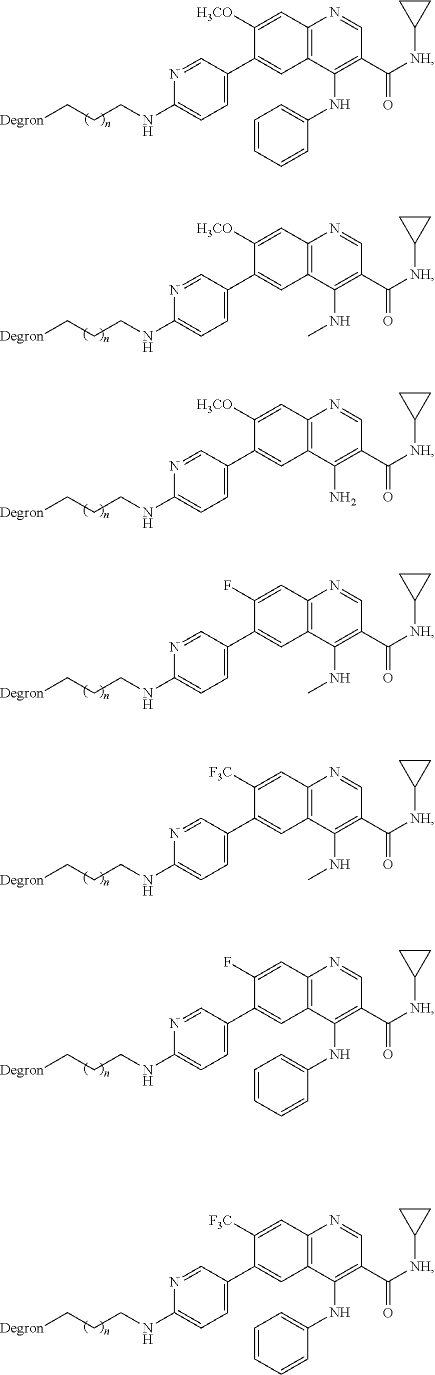

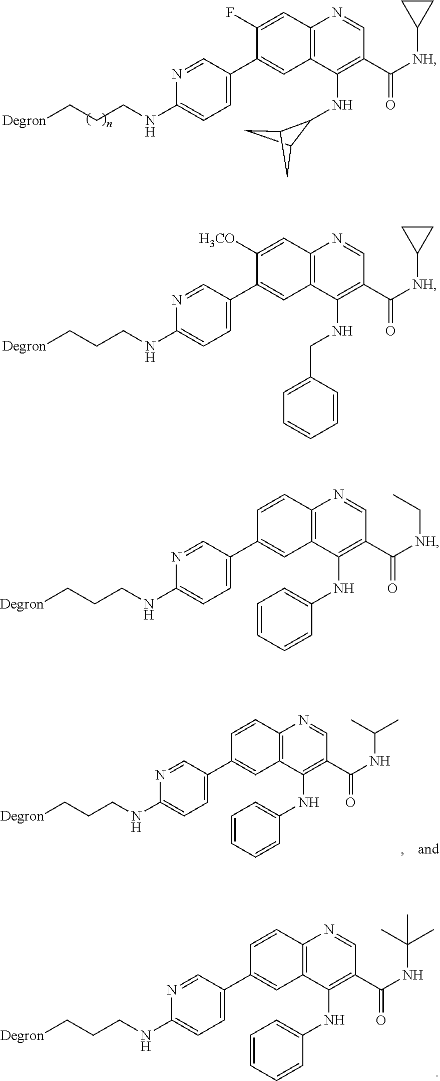

In some embodiments, the compound of Formula V is selected from:

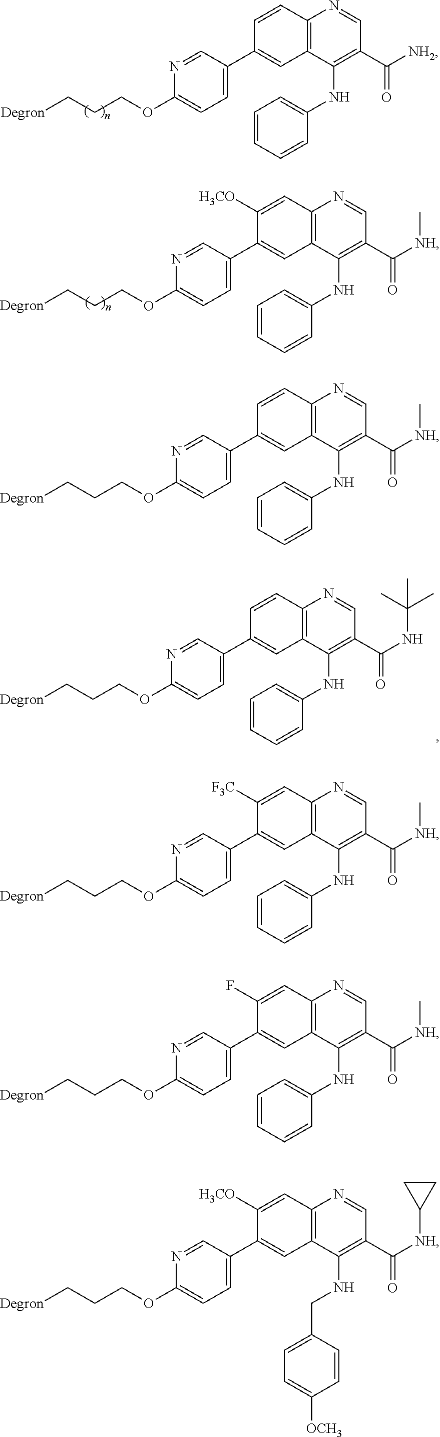

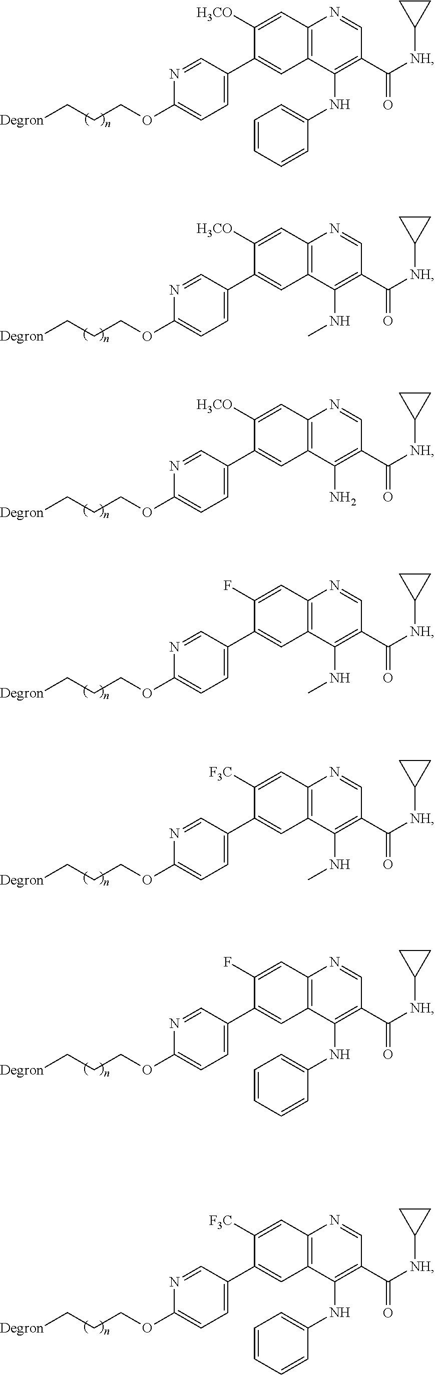

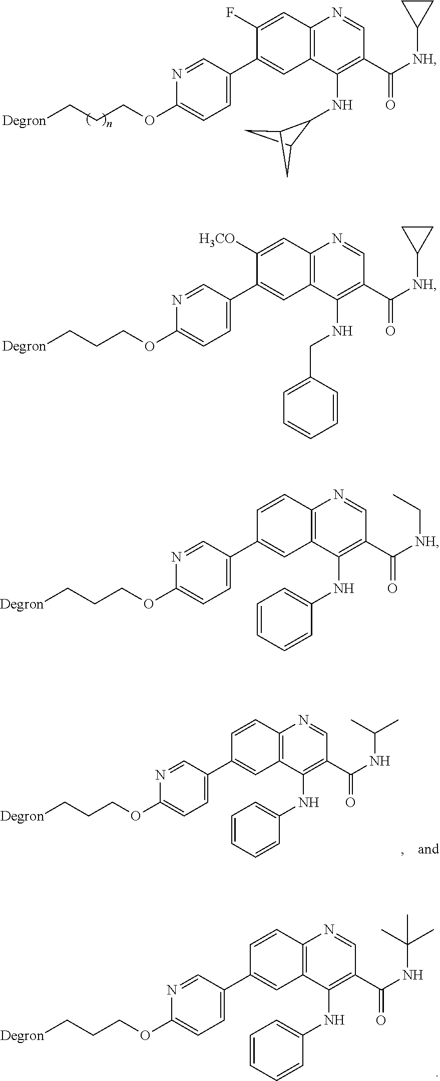

In some embodiments, the compound of Formula V is selected from:

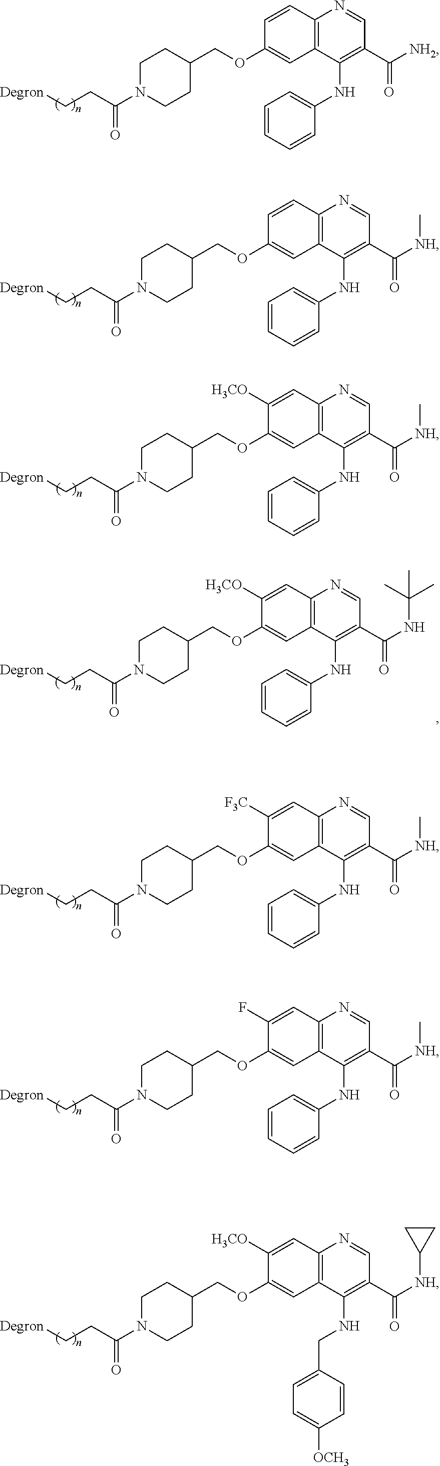

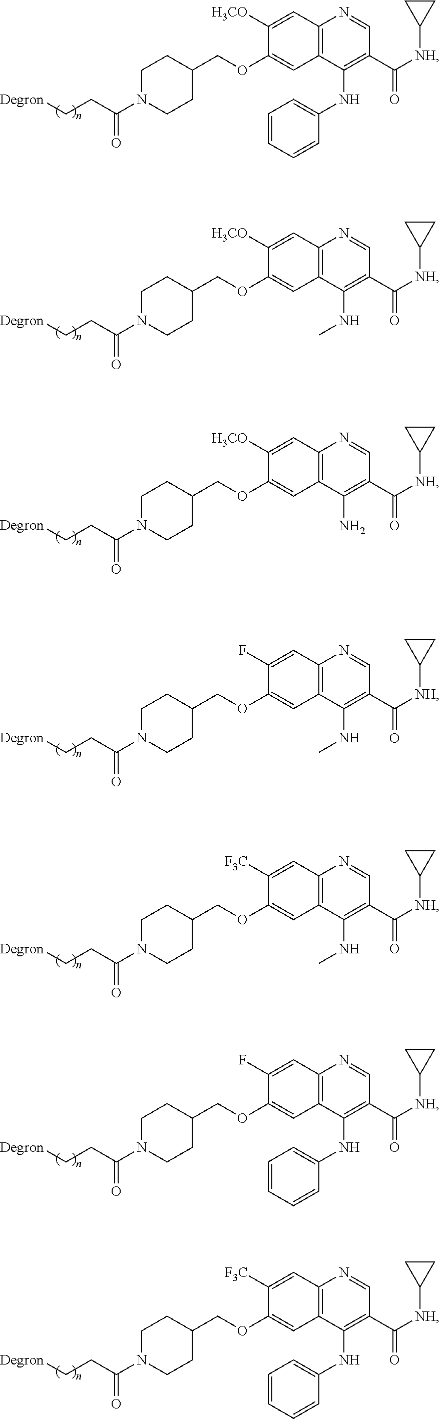

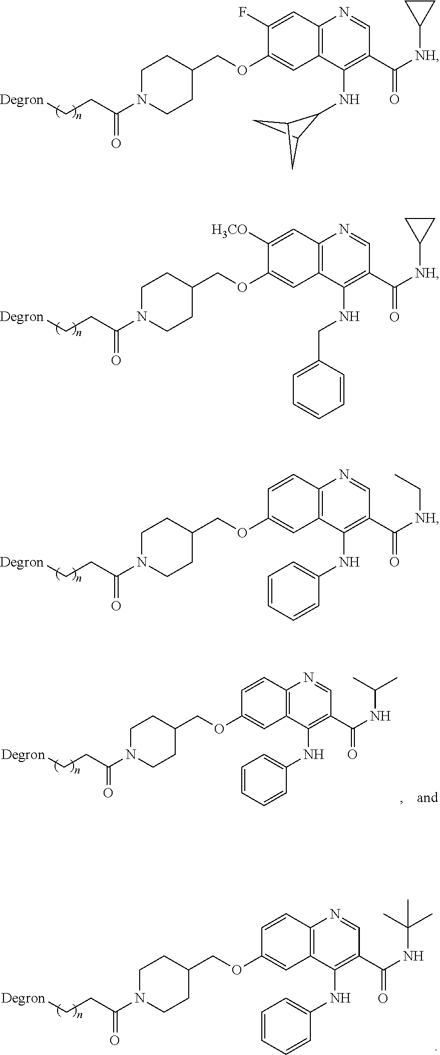

In some embodiments, the compound of Formula V is selected from:

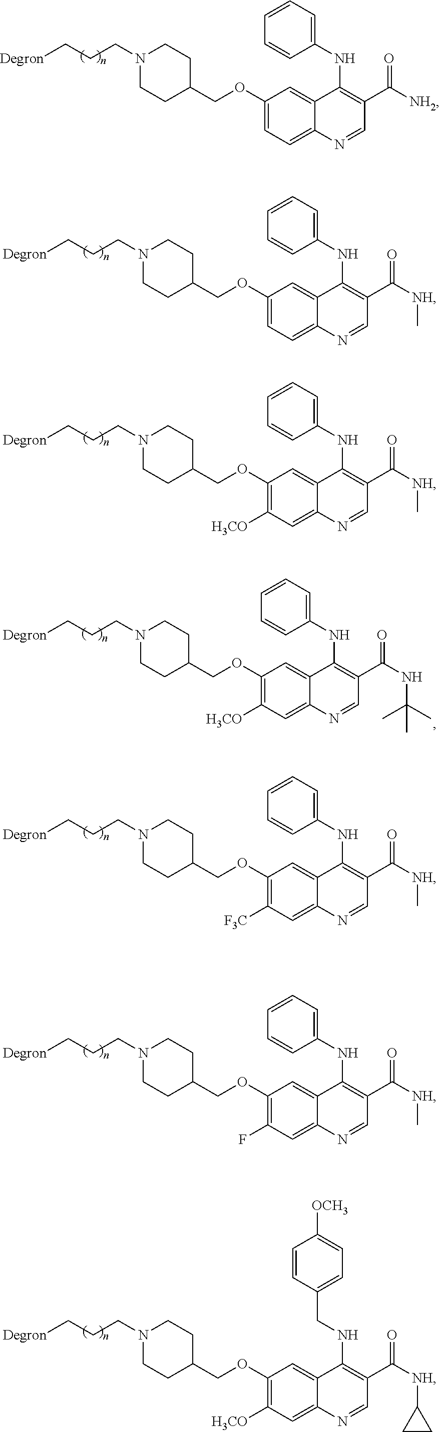

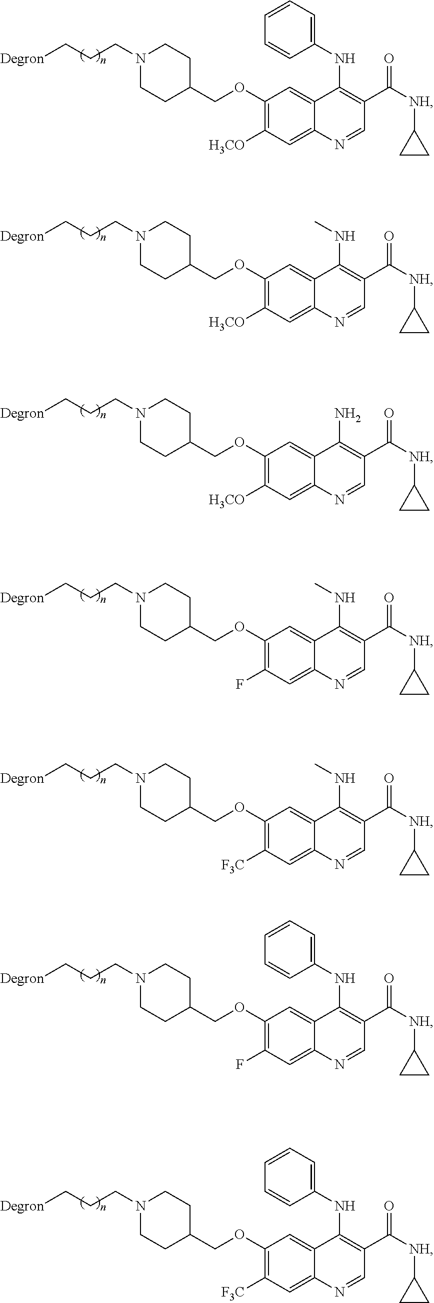

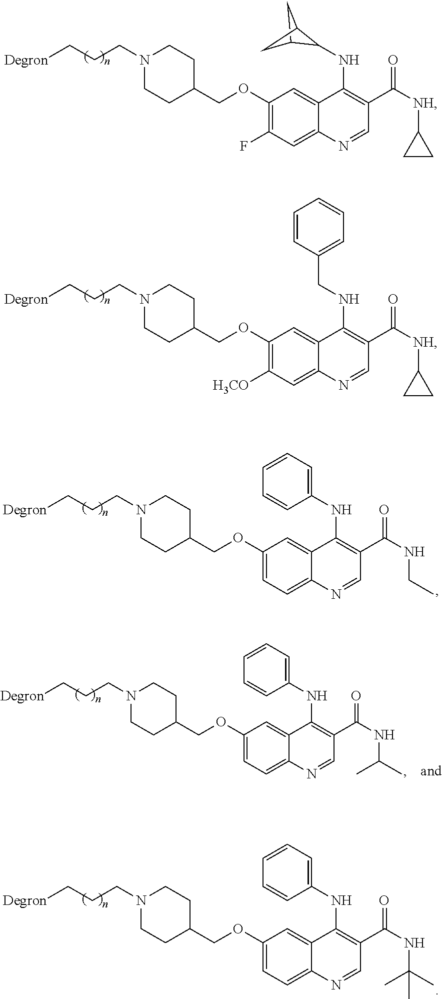

In some embodiments, the compound of Formula V is selected from:

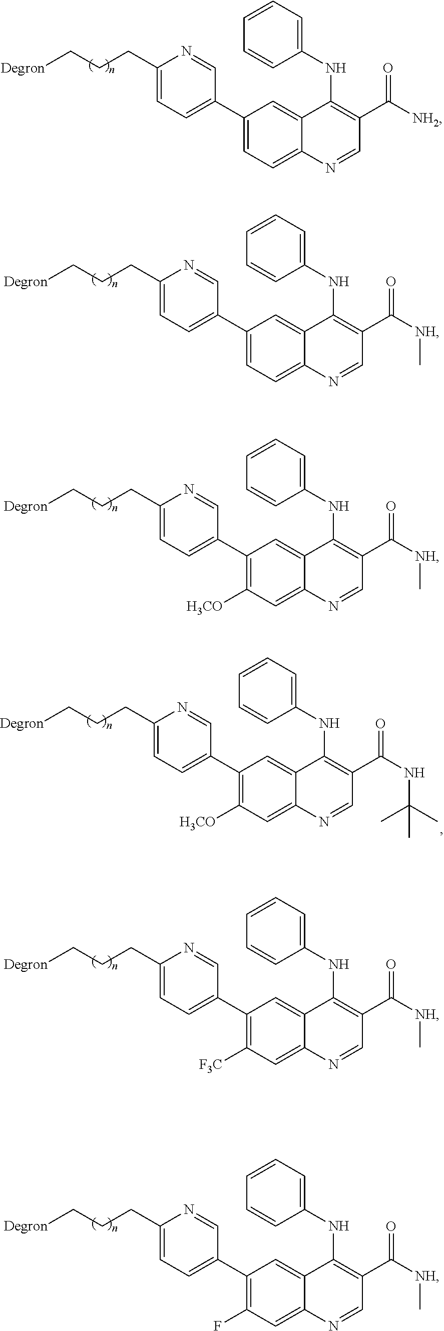

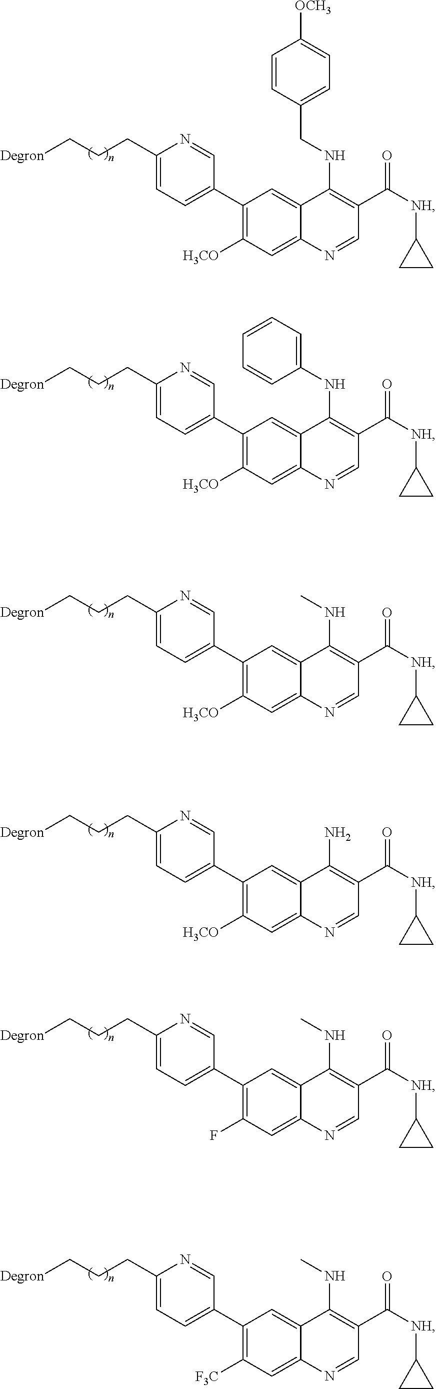

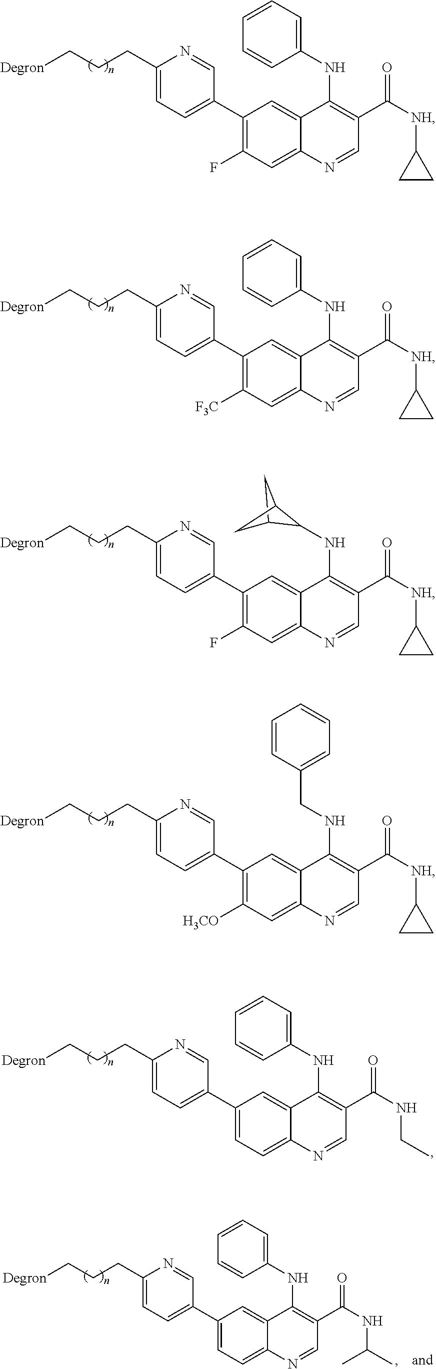

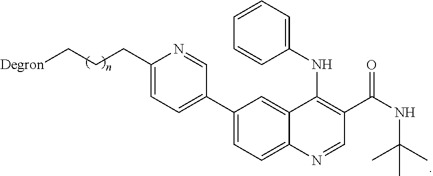

In some embodiments, the compound of Formula V is selected from:

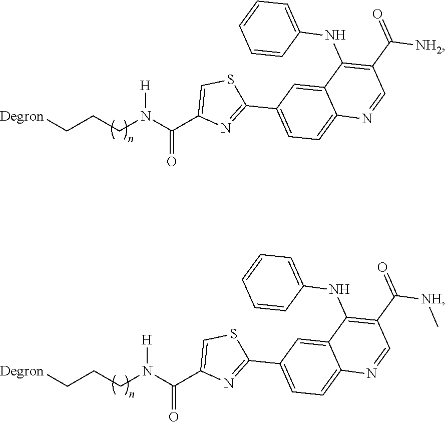

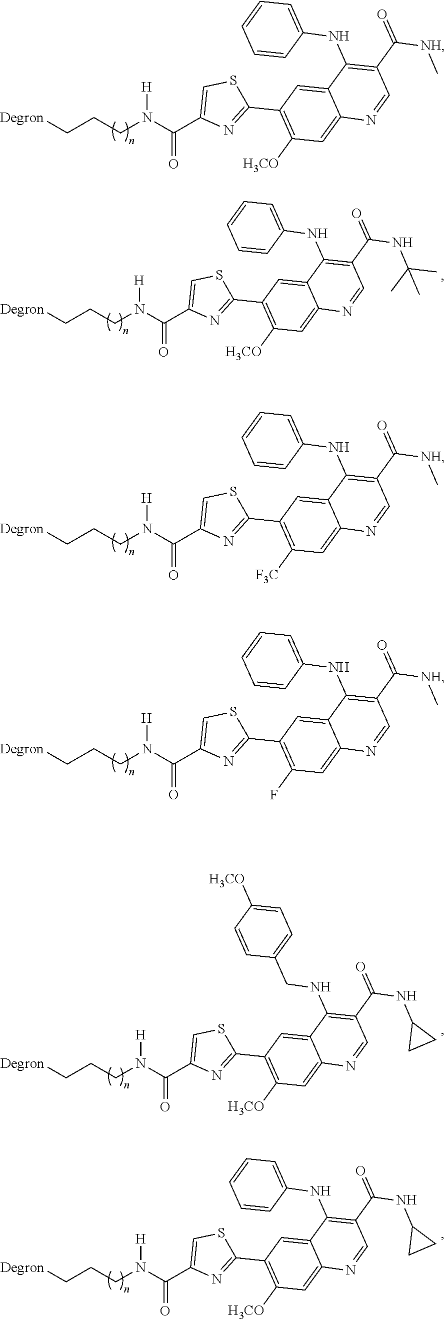

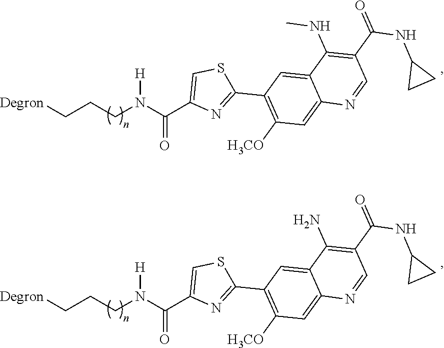

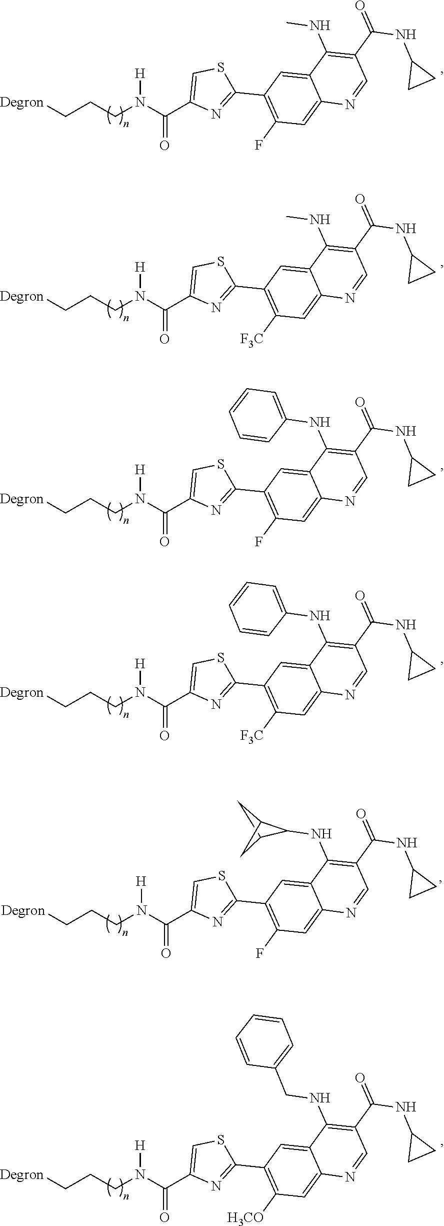

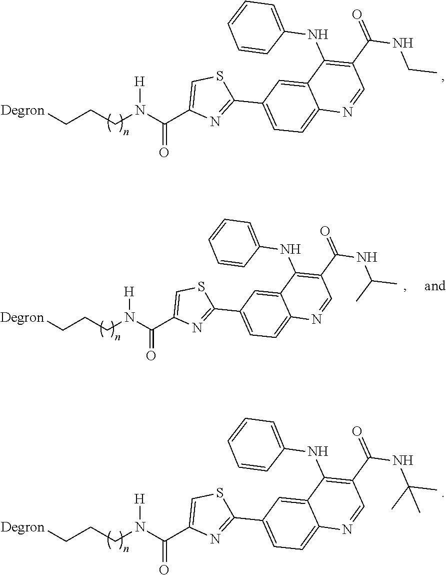

In some embodiments, the compound of Formula V is selected from:

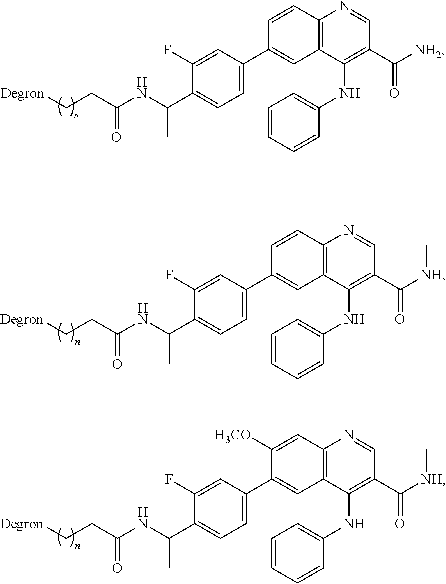

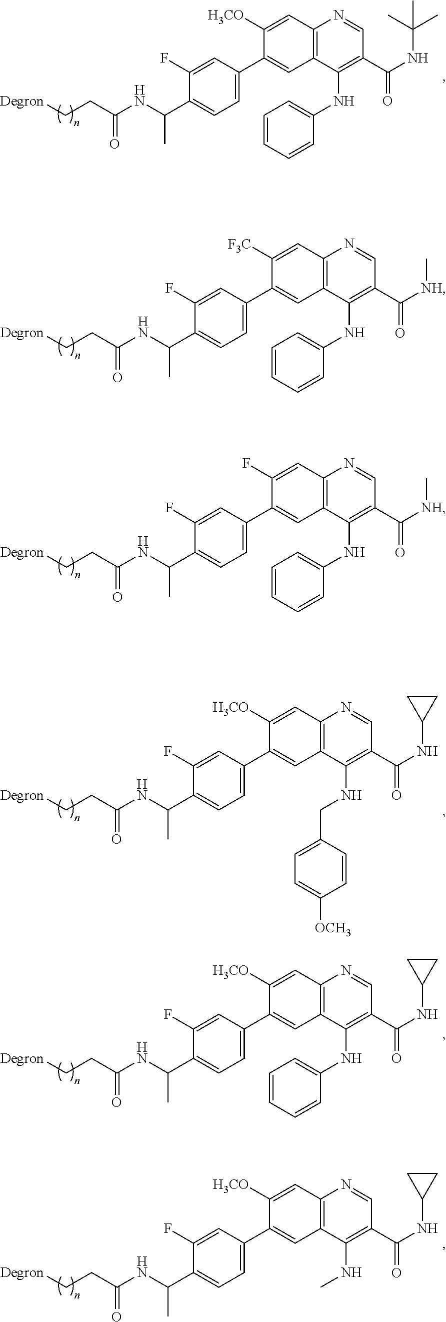

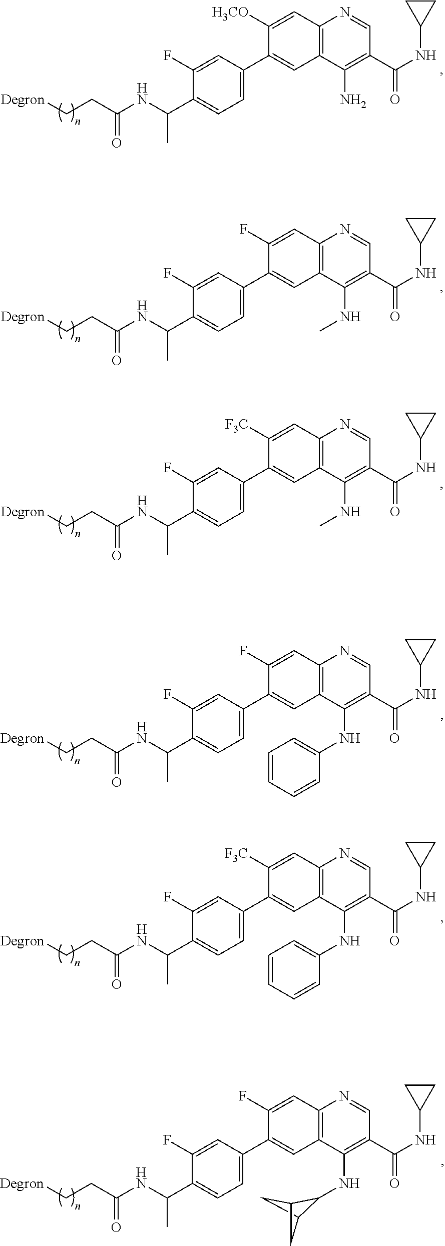

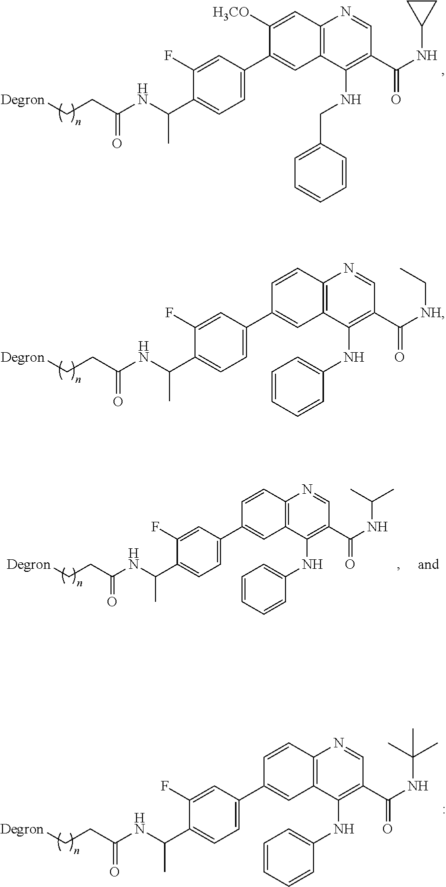

In some embodiments, the compound of Formula V is selected from:

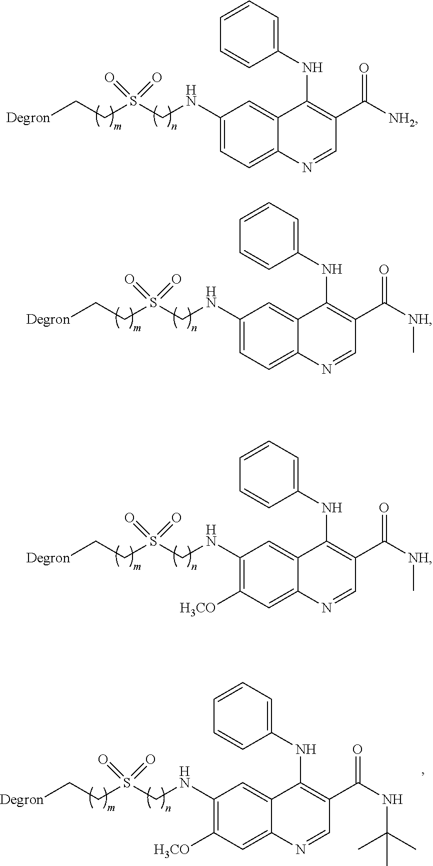

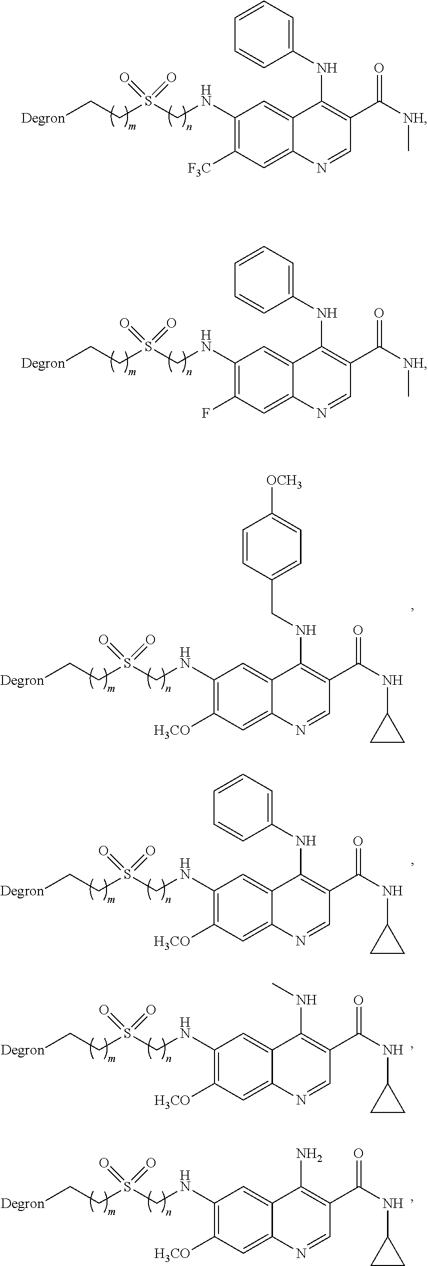

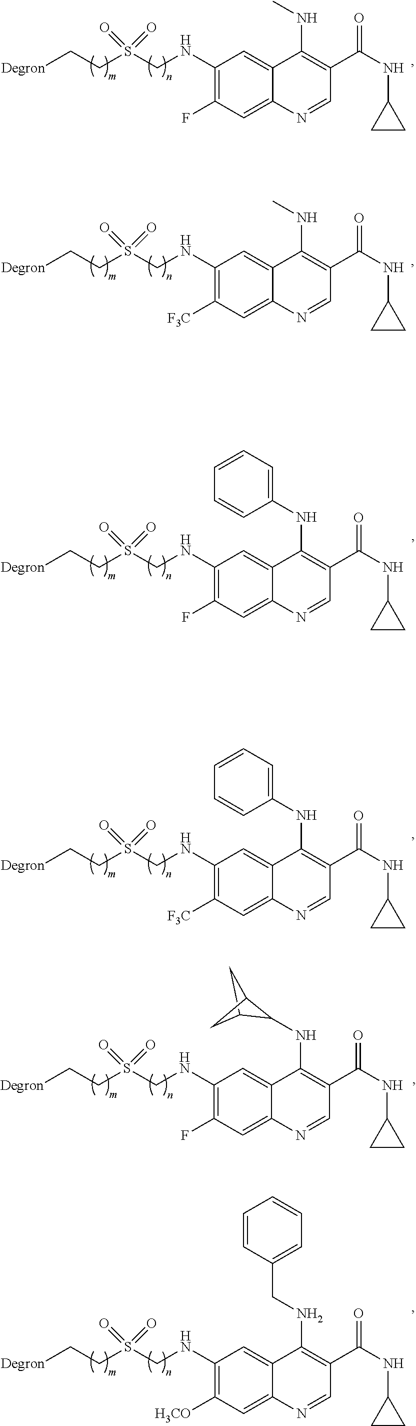

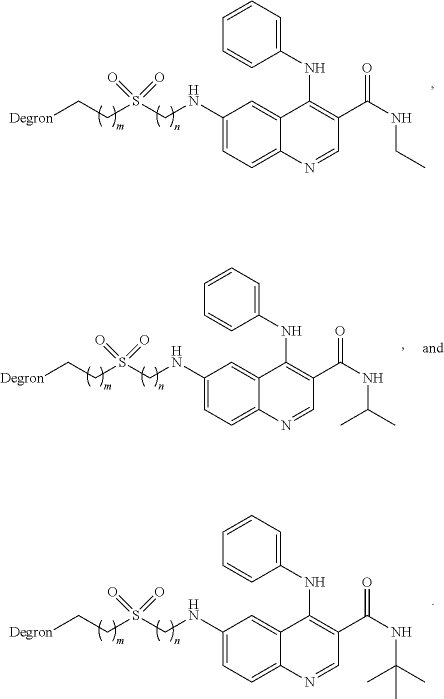

In some embodiments, the compound of Formula V is selected from:

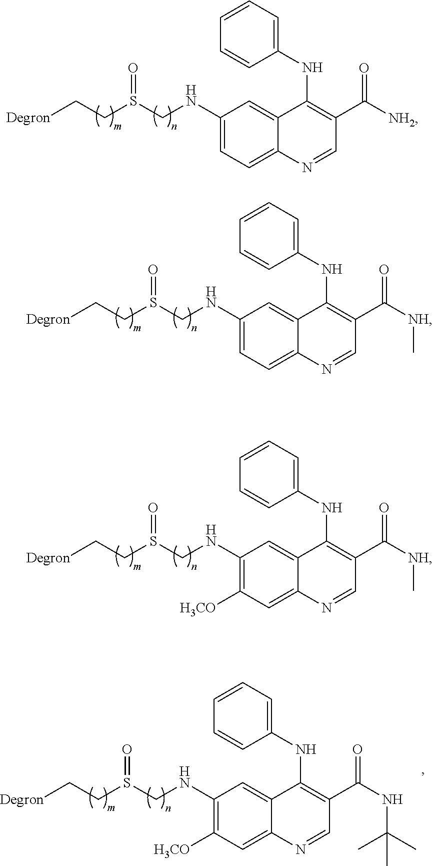

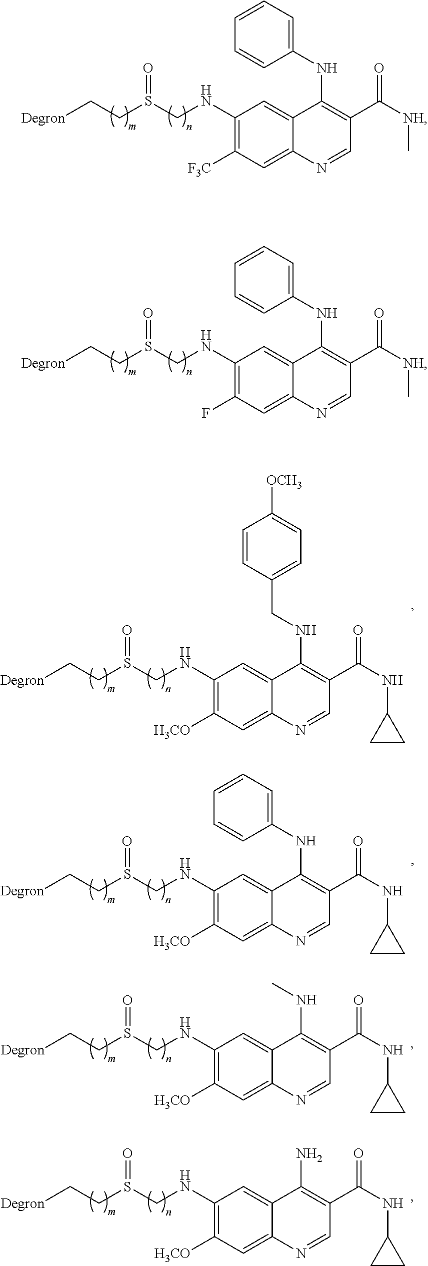

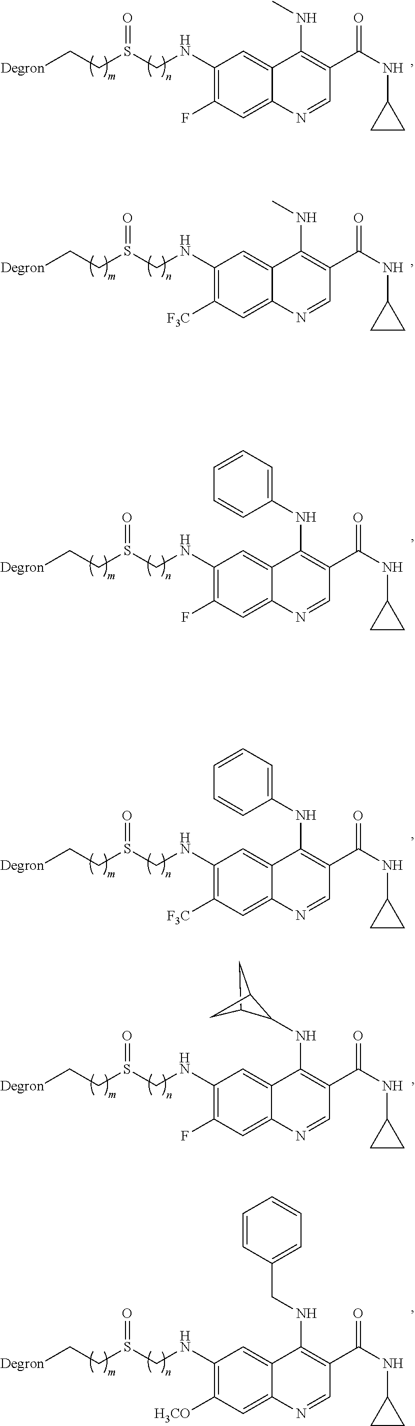

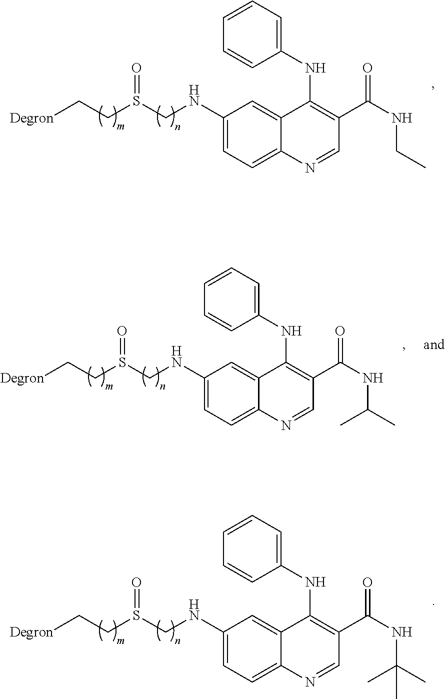

In some embodiments, the compound of Formula V is selected from:

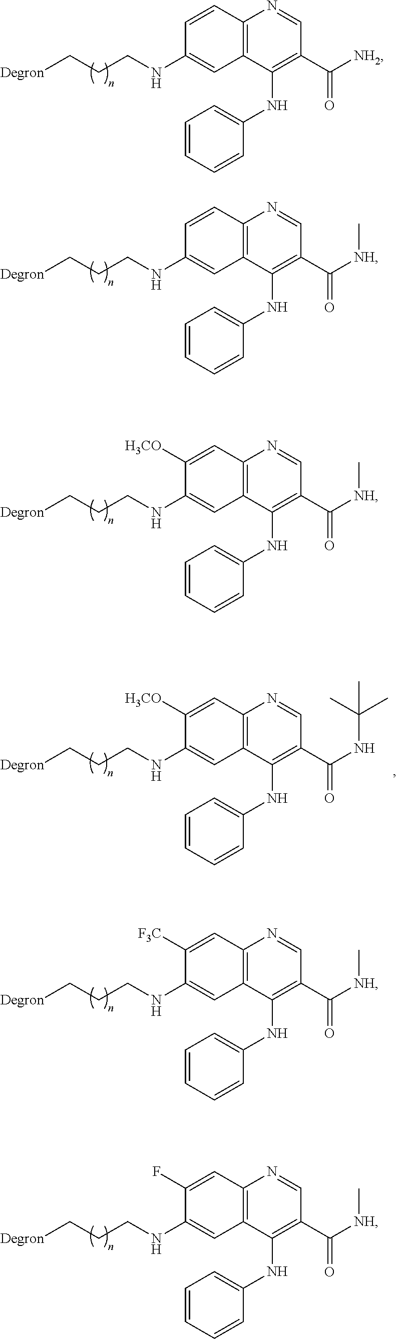

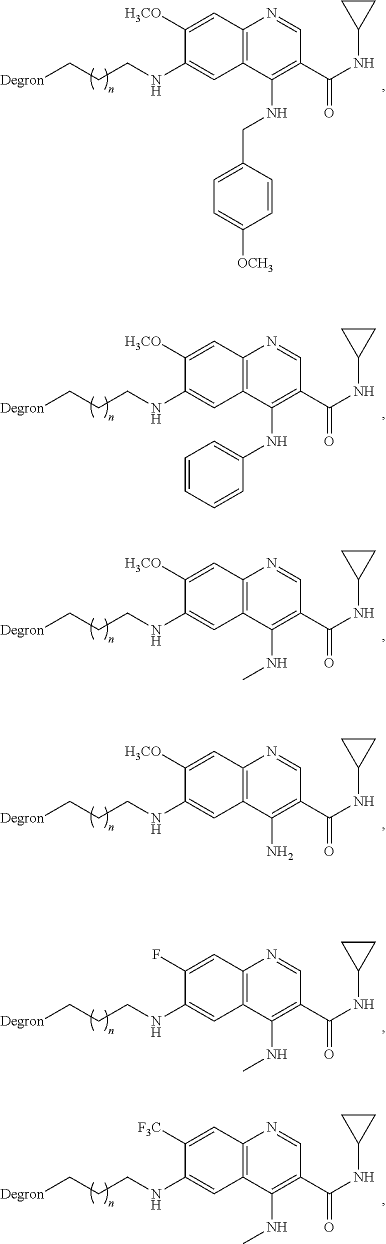

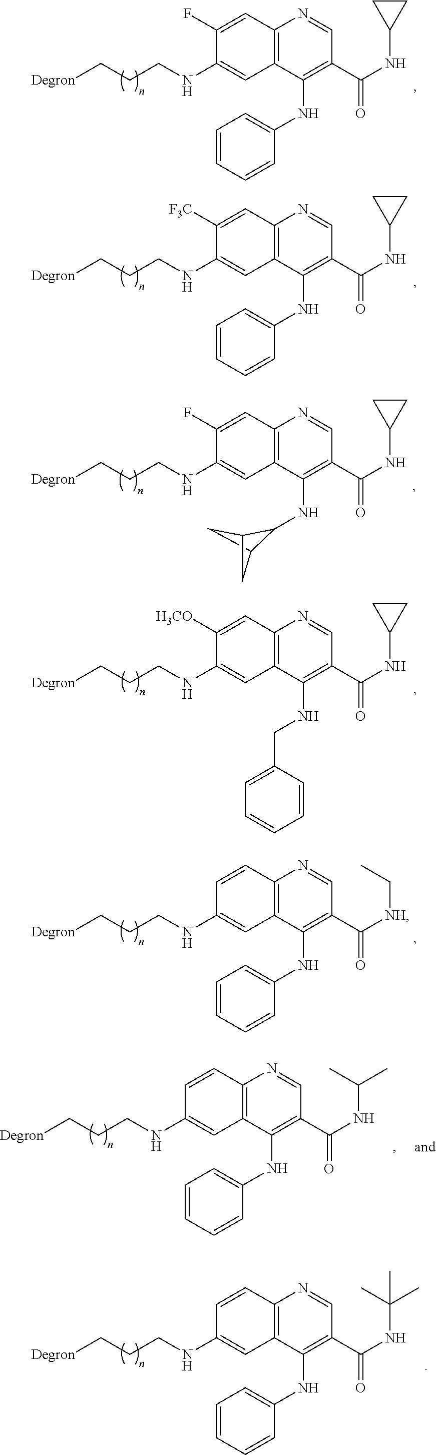

In some embodiments, the compound of Formula V is selected from:

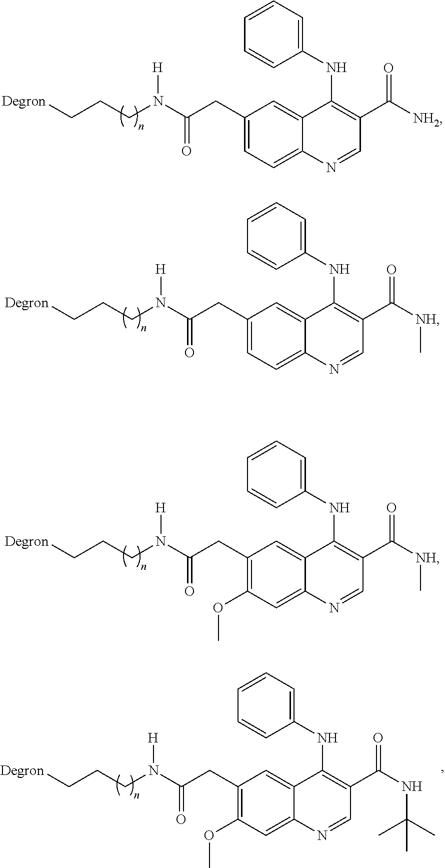

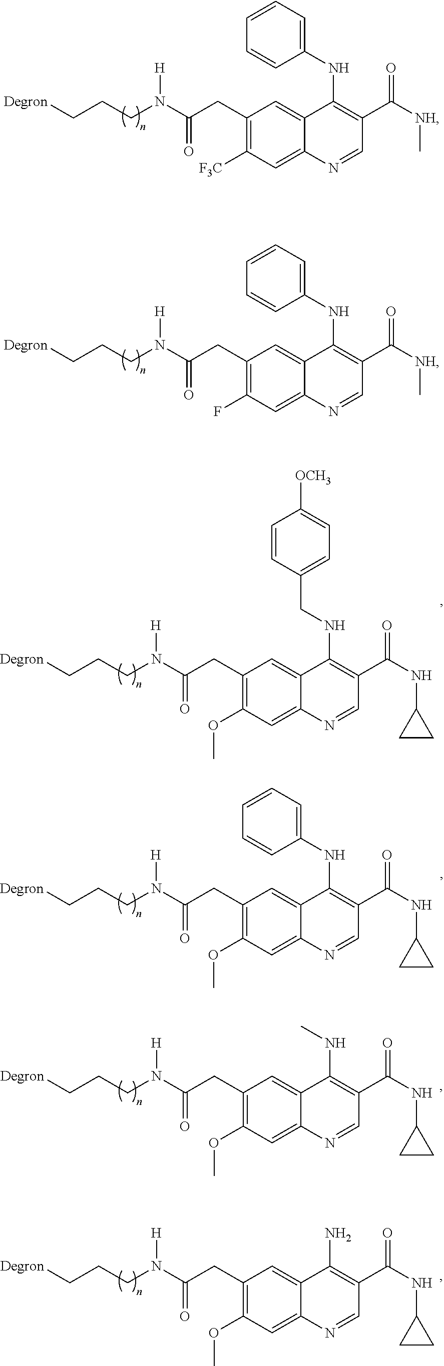

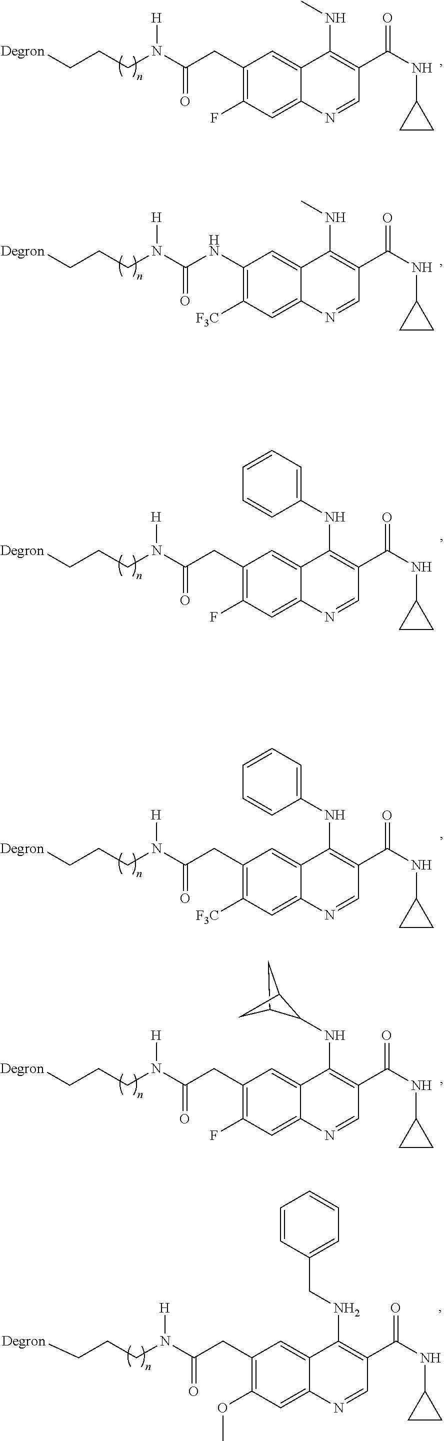

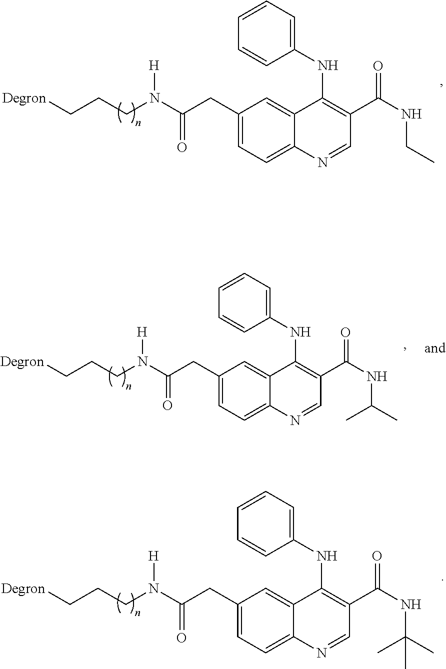

In some embodiments, the compound of Formula V is selected from:

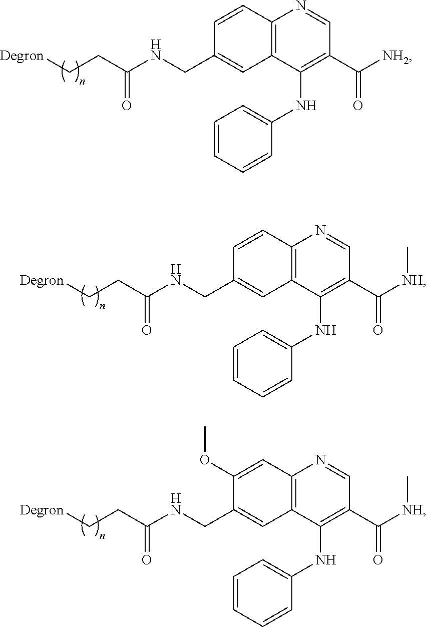

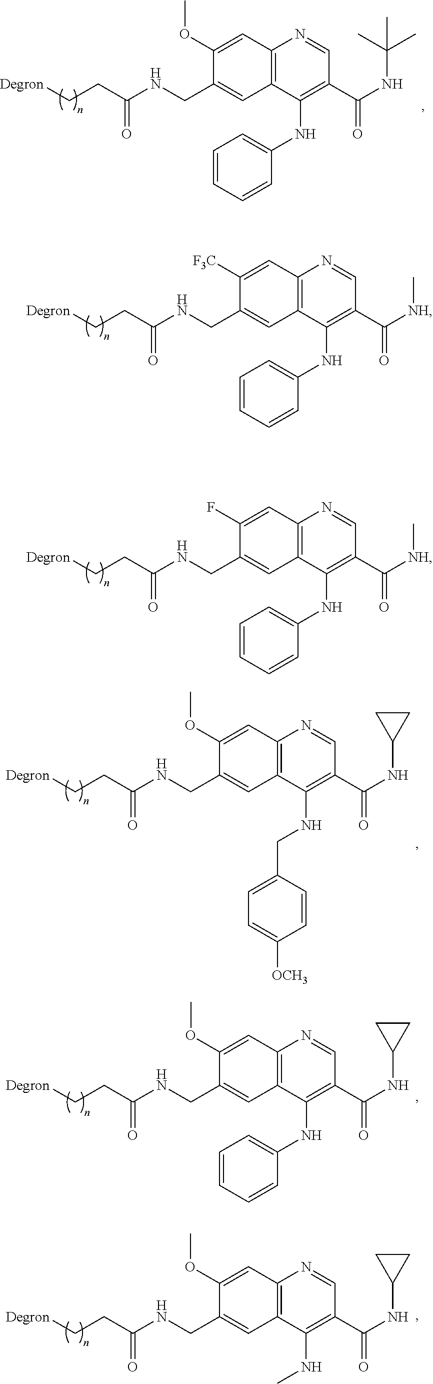

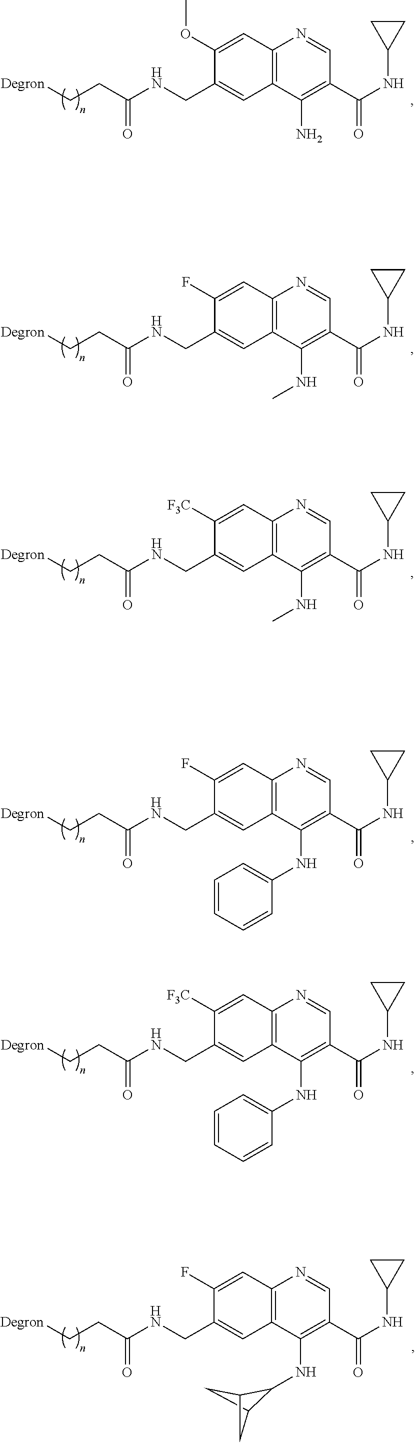

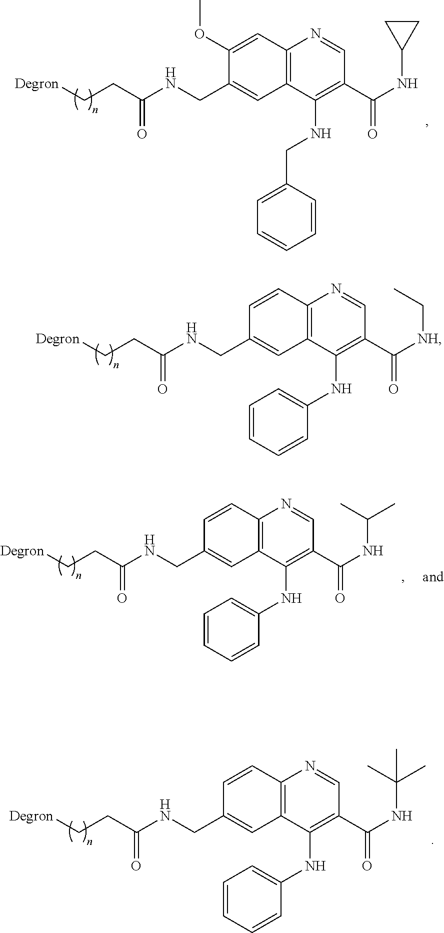

In some embodiments, the compound of Formula V is selected from:

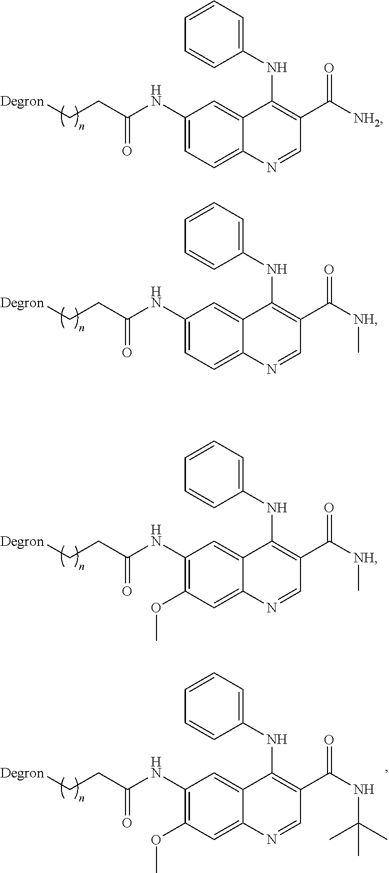

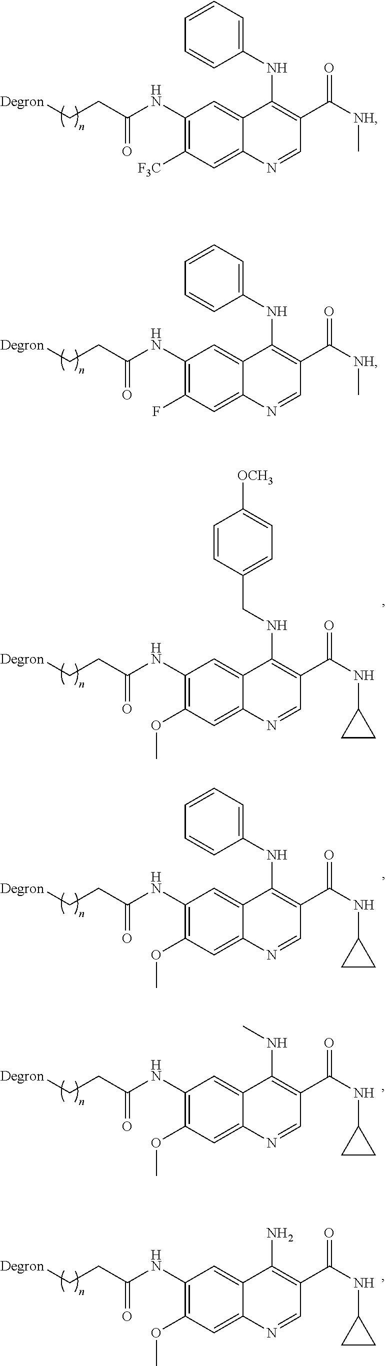

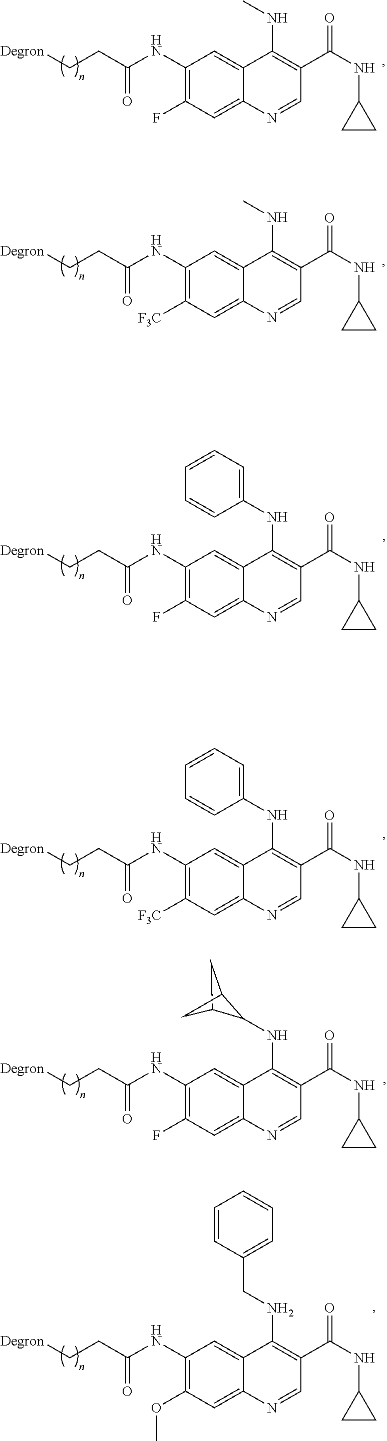

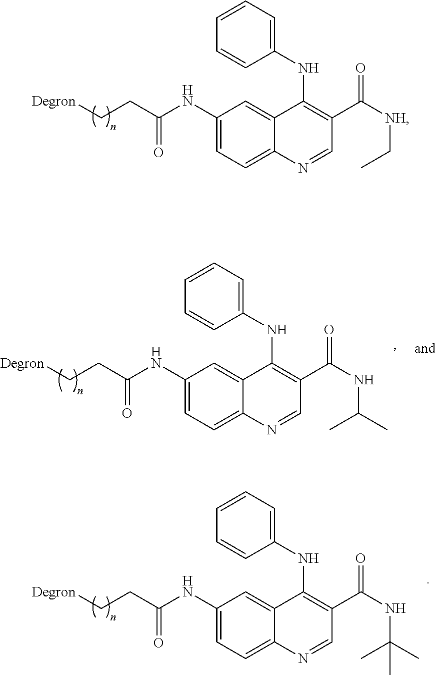

### Non-Limiting Examples of MTH1-Binding Compounds of Formula VI

In some embodiments, a compound of Formula VI is selected from:

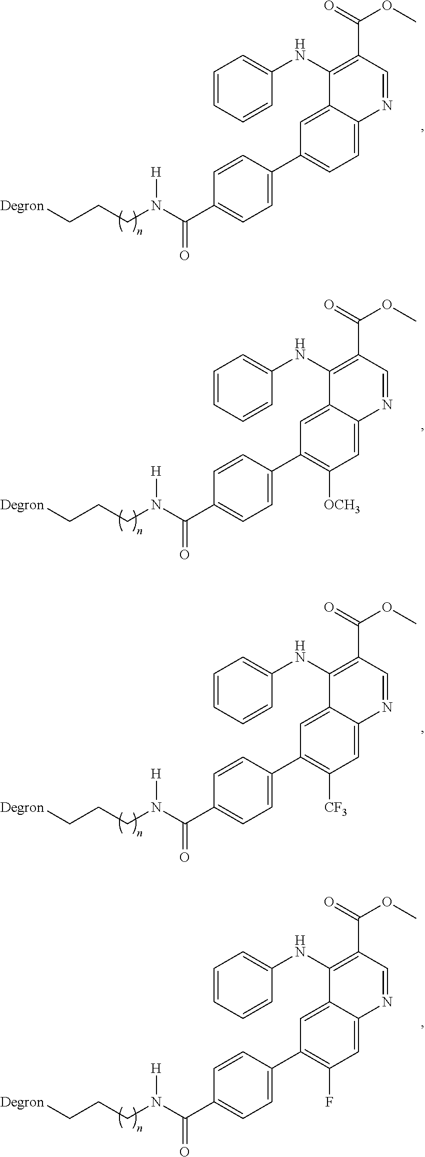

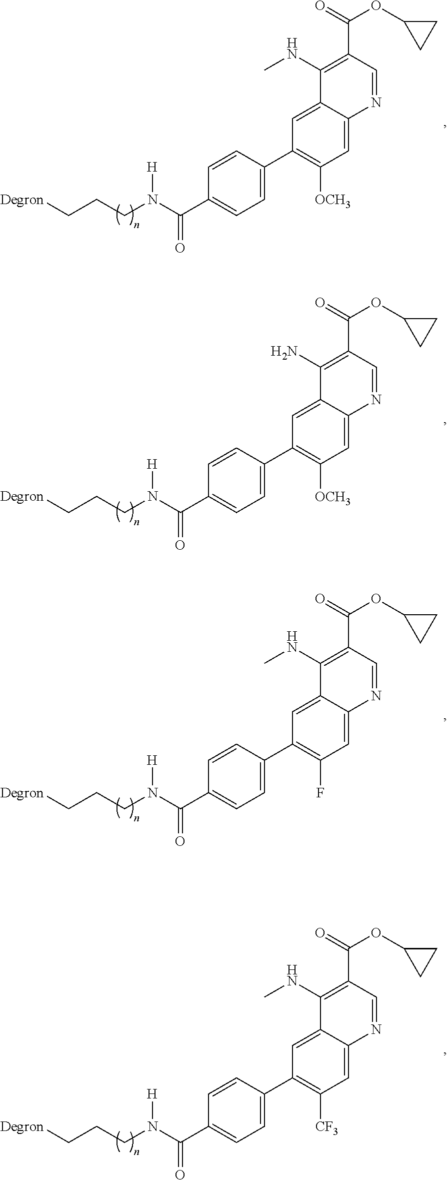

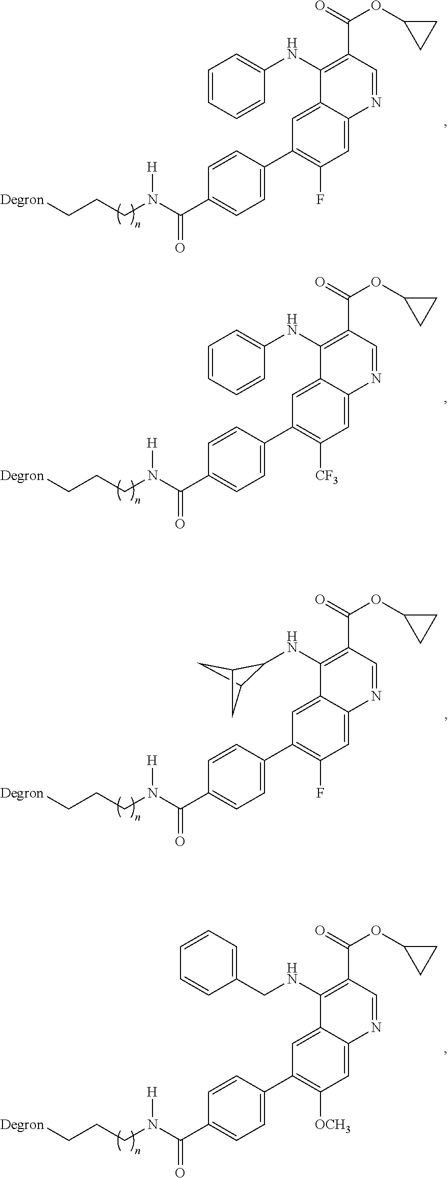

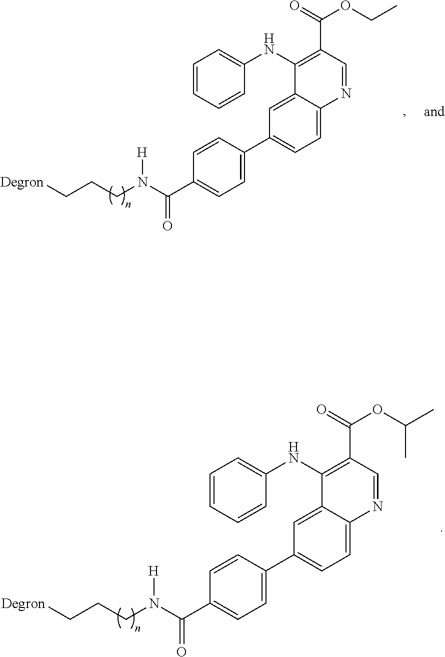

### III. Methods of Treatment

Any of the BRD9-binding or MTH1-binding compounds described herein can be used in an effective amount to treat a host, including a human, in need thereof, optionally in a pharmaceutically acceptable carrier to treat any of the disorders described herein, and in particular, those which are mediated by the respective protein BRD9 or MTH1 or a fragment thereof. In certain embodiments, the method comprises administering an effective amount of the active compound or its salt as described herein, optionally including a pharmaceutically acceptable excipient, carrier, or adjuvant (i.e., a pharmaceutically acceptable composition), optionally in combination or alternation with another bioactive agent or combination of agents.

In one embodiment a compound of Formula I is used to treat a disorder described herein.

In one embodiment a compound of Formula II is used to treat a disorder described herein.

In one embodiment a compound of Formula III is used to treat a disorder described herein.

In one embodiment a compound of Formula IV is used to treat a disorder described herein.

In one embodiment a compound of Formula V is used to treat a disorder described herein.

In one embodiment a compound of Formula VI is used to treat a disorder described herein.

In one embodiment the disorder treated by a compound of the present invention involves angiogenesis. In one embodiment, the disorder treated by a compound of the present invention is used to treat cancer.

The compounds described herein are useful in the treatment of cancer, including hematological cancers and solid cancers. Hematological cancers that can be treated with the compounds described herein include, but are not limited to, leukemia, lymphoma, and multiple myeloma. In certain embodiments, the hematological cancer is acute myelogenous leukemia (AML), acute lymphoblastic leukemia (ALL), lymphoblastic T-cell leukemia, chronic myelogenous leukemia (CIVIL), chronic lymphocytic leukemia (CLL), hairy-cell leukemia, chronic neutrophilic leukemia (CNL), acute lymphoblastic T-cell leukemia, acute monocytic leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukemia (MLL), erythroleukemia, malignant lymphoma, Hodgkins lymphoma, non-Hodgkins lymphoma, lymphoblastic T-cell lymphoma, Burkitt's lymphoma, follicular lymphoma, B cell acute lymphoblastic leukemia, diffuse large B cell lymphoma, Myc and B-Cell Leukemia (BCL)2 and/or BCL6 rearrangements/overexpression [double- and triple-hit lymphoma], myelodysplastic/myeloproliferative neoplasm, mantle cell lymphoma including bortezomib resistant mantle cell lymphoma.

Solid tumors that can be treated with the compounds described herein include, but are not limited to lung cancers, including small cell lung cancer (SCLC) and non-small cell lung cancer (NSCLC), breast cancers including inflammatory breast cancer, ER-positive breast cancer including tamoxifen resistant ER-positive breast cancer, and triple negative breast cancer, colon cancers, midline carcinomas, liver cancers, renal cancers, prostate cancers including castrate resistant prostate cancer (CRPC), brain cancers including gliomas, glioblastomas, neuroblastoma, and medulloblastoma including MYC-amplified medulloblastoma, colorectal cancers, Wilm's tumor, Ewing's sarcoma, rhabdomyosarcomas, ependymomas, head and neck cancers, melanomas, squamous cell carcinomas, ovarian cancers, pancreatic cancers including pancreatic ductal adenocarcinomas (PDAC) and pancreatic neuroendocrine tumors (PanNET), osteosarcomas, giant cell tumors of bone, thyroid cancers, bladder cancers, urothelial cancers, vulval cancers, cervical cancers, endometrial cancers, mesotheliomas, esophageal cancers, salivary gland cancers, gastric cancers, nasopharangeal cancers, buccal cancers, cancers of the mouth, GIST (gastrointestinal stromal tumors), NUT-midline carcinomas, testicular cancers, squamous cell carcinomas, hepatocellular carcinomas (HCC), MYCN driven solid tumors, and NUT midline carcinomas (NMC).

In one embodiment, a compound or its corresponding pharmaceutically acceptable salt, isotopic derivative, or prodrug as described herein can be used in an effective amount to treat a host, for example a human, with a lymphoma or lymphocytic or myelocytic proliferation disorder or abnormality. For example, a compound as described herein can be administered to a host suffering from a Hodgkin's Lymphoma or a Non-Hodgkin's Lymphoma. For example, the host can be suffering from a Non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); diffuse small-cleaved cell lymphoma (DSCCL); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; Langerhans cell histiocytosis; or Waldenstrom's Macroglobulinemia.

In another embodiment, a compound or its corresponding pharmaceutically acceptable salt, isotopic derivative, or prodrug as described herein can be used in an effective amount to treat a host, for example a human, with a Hodgkin's lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin's Lymphoma; or Nodular Lymphocyte Predominant HL.

In certain embodiments, the condition treated with a compound of the present invention is a disorder related to abnormal cellular proliferation. Abnormal cellular proliferation, notably hyperproliferation, can occur as a result of a wide variety of factors, including genetic mutation, infection, exposure to toxins, autoimmune disorders, and benign or malignant tumor induction. Abnormal proliferation of B-cells, T-cells, and/or NK cells can result in a wide range of diseases such as cancer, proliferative disorders and inflammatory/immune diseases. A host, for example a human, afflicted with any of these disorders can be treated with an effective amount of a compound as described herein to achieve a decrease in symptoms (palliative agent) or a decrease in the underlying disease (a disease modifying agent).

In one embodiment, a compound or its corresponding pharmaceutically acceptable salt, isotopic derivative, or prodrug as described herein can be used in an effective amount to treat a host, for example a human, with a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; diffuse poorly differentiated lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin's lymphoma.

In one embodiment, a compound or its corresponding pharmaceutically acceptable salt, isotopic derivative, or prodrug as described herein can be used in an effective amount to treat a host, for example a human, with a T-cell or NK-cell lymphoma such as, but not limited to: anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In one embodiment, a compound or its corresponding pharmaceutically acceptable salt, isotopic derivative, or prodrug as described herein can be used to treat a host, for example a human, with leukemia. For example, the host may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In one embodiment, the patient suffers from an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma. Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

A compound or its pharmaceutically acceptable salt, isotopic analog, or prodrug as described herein can be used in an effective amount to treat a host, for example a human, with a proliferative condition such as myeloproliferative disorder (MPD), polycythemia vera (PV), essential thrombocythemia (ET), myeloid metaplasia with myelofibrosis (MMM), chronic myelomonocytic leukemia (CMML), hypereosinophilic syndrome (HES), system mast cell disease (SMCD), and the like. In another embodiment, a compound provided herein is useful for the treatment of primary myelofibrosis, post-polycythemia vera myelofibrosis, post-essential thrombocythemia myelofibrosis, and secondary acute myelogenous leukemia.

In one embodiment, a compound or its pharmaceutically acceptable salt, isotopic analog, or prodrug as described herein can be used in an effective amount to treat a host, for example a human, with a myelodysplastic syndrome (MDS) such as, but not limited to: refractory cytopenia with unilineage dysplasia, refractory anemia with ring sideroblasts (RARS), refractory anemia with ring sideroblasts-thrombocytosis (RARS-t), refractory cytopenia with multilineage dyslplasia (RCMD) including RCMD with multilineage dysplasia and ring sideroblasts (RCMD-RS), Refractory amenias with excess blasts I (RAEB-I) and II (RAEB-II), 5q-syndrome, refractory cytopenia of childhood, and the like.

The term “neoplasia” or “cancer” is used to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CIVIL), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin's lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin's lymphoma. In one embodiment, the disorder is adenoid cystic carcinoma. In one embodiment, the disorder is NUT midline carcinoma.

Also provided herein are methods of modulating the activity of a T-cell expressing a chimeric antigen receptor (CAR) comprising administering to a subject that has previously been administered a T-cell expressing a CAR a compound described herein, wherein the CAR comprises an MTH1 protein fragment or BRD9 protein fragment. In some embodiments, the CAR comprises an MTH1 protein fragment or BRD9 protein fragment that is capable of being bound by a compound described herein, which subjects the CAR to degradation through ubiquitination. In some embodiments, the CAR comprises an amino acid sequence derived from the human MTH1 protein (UniProtKB—P36639 (8ODP_HUMAN)) incorporated herein by reference) or variant thereof. In some embodiments, the CAR comprises an amino acid sequence derived from the human BRD9 protein (UnitProtKB-Q9H8M2 (BRD(-HUMAN)) incorporated herein by reference) or variant thereof. By including an MTH1 protein fragment or BRD9 protein fragment capable of being bound by a compound described herein and subsequently degraded through ubiquitination, the activity of a T-cell or other immune effector cell encoding a CAR can be reversibly controlled, allowing for the modulation of the immune response while sparing the immune effector cell itself. In some embodiments, by administering a compound described herein to an individual who has received a CAR expressing an MTH1 protein fragment or BRD9 protein fragment, adverse effects associated with CAR immune effector cell therapies such as adverse inflammatory responses, including cytokine release syndrome, can be controlled. Methods for preparing T-cells comprising a CAR comprising an amino acid sequences capable of being bound by a compound comprising an E3 ubiquitin ligase targeting moiety (Degron) that is linked to a targeting ligand, and subsequently ubiquitinated, are described in, e.g., U.S. Publication No. 20180169109 and PCT Publication No. WO2018148440, incorporated herein by reference.

### IV. Combination Therapy

Any of the BRD9-binding or MTH1-binding compounds described herein can be used in an effective amount alone or in combination with a bioactive agent to treat a host such as a human with a disorder as described herein.

The term “bioactive agent” is used to describe an agent, other than the compound according to the present invention, which can be used in combination or alternation with a compound of the present invention to achieve a desired result of therapy. In one embodiment, the compound of the present invention and the bioactive agent are administered in a manner that they are active in vivo during overlapping time periods, for example, have time-period overlapping Cmax, Tmax, AUC or other pharmacokinetic parameter. In another embodiment, the compound of the present invention and the bioactive agent are administered to a host in need thereof that do not have overlapping pharmacokinetic parameter, however, one has a therapeutic impact on the therapeutic efficacy of the other.

In one aspect of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (Opdivo), pembrolizumab (Keytruda), pidilizumab, AMP-224 (AstraZeneca and Medlmmune), PF-06801591 (Pfizer), MEDI0680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (Tecentriq), durvalumab (AstraZeneca and Medlmmune), KN035 (Alphamab), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and Medlmmune), AGEN1884 and AGEN2041 (Agenus). LAG-3 checkpoint inhibitors, include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). An example of a TIM-3 inhibitor is TSR-022 (Tesaro).

In another embodiment, an active compounds described herein can be administered in an effective amount for the treatment of abnormal tissue of the male reproductive system such as prostate or testicular cancer, in combination or alternation with an effective amount of an androgen (such as testosterone) inhibitor including but not limited to a selective androgen receptor modulator, a selective androgen receptor degrader, a complete androgen receptor degrader, or another form of partial or complete androgen antagonist. In one embodiment, the prostate or testicular cancer is androgen-resistant. Non-limiting examples of anti-androgen compounds are provided in WO 2011/156518 and U.S. Pat. Nos. 8,455,534 and 8,299,112. Additional non-limiting examples of anti-androgen compounds include: enzalutamide, apalutamide, cyproterone acetate, chlormadinone acetate, spironolactone, canrenone, drospirenone, ketoconazole, topilutamide, abiraterone acetate, and cimetidine.

In one embodiment, the bioactive agent is an ALK inhibitor. Examples of ALK inhibitors include but are not limited to Crizotinib, Alectinib, ceritinib, TAE684 (NVP-TAE684), GSK1838705A, AZD3463, ASP3026, PF-06463922, entrectinib (RXDX-101), and AP26113.

In one embodiment, the bioactive agent is an EGFR inhibitor. Examples of EGFR inhibitors include erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775 (Pfizer), icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer).

In one embodiment, the bioactive agent is an HER-2 inhibitor. Examples of HER-2 inhibitors include trastuzumab, lapatinib, ado-trastuzumab emtansine, and pertuzumab.

In one embodiment, the bioactive agent is a CD20 inhibitor. Examples of CD20 inhibitors include obinutuzumab, rituximab, fatumumab, ibritumomab, tositumomab, and ocrelizumab. In one embodiment, the bioactive agent is a JAK3 inhibitor. Examples of JAK3 inhibitors include tasocitinib.

In one embodiment, the bioactive agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide) (navitoclax), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-54 [2-(1-methylethyl)phenyl]methyl]benzamide), Apogossypolone (ApoG2), HA14-1, AT101, sabutoclax, gambogic acid, or G3139 (Oblimersen).

In one embodiment, the bioactive agent is a kinase inhibitor. In one embodiment, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

Examples of PI3 kinase inhibitors include but are not limited to Wortmannin, demethoxyviridin, perifosine, idelalisib, Pictilisib, Palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2 S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-(0-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methyl sulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′44-(4,6-di-4-morpholinyl-1,3,5-triazin-2-yl)phenyl]urea) (gedatolisib), LY3023414, BEZ235 (2-Methyl-2-4-[3-methyl-2-oxo-8-(quinolin-3-yl)-2,3-dihydro-1H-imidazo[4,5-c]quinolin-1-yl]phenyl propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3 aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl]acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422), and the structure described in WO2014/071109.

Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acryl amide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-(3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors include, for example, Cerdulatinib (4-(cyclopropylamino)-2-((4-(4-(ethyl sulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3 (4H)-one), piceatannol (3-Hydroxyresveratol), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

In one embodiment, the bioactive agent is a MEK inhibitor. MEK inhibitors are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-(3,4-difluoro-2-[(2-fluoro-4-iodophenyl)amino]phenyl}carbonyl)-3-[(2 S)-piperidin-2-yl]azetidin-3-ol), refametinib/BAY869766/RDEA1 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H, 8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide), UO126-EtOH, PD184352 (CI-1040), GDC-0623, BI-847325, cobimetinib, PD98059, BIX 02189, BIX 02188, binimetinib, SL-327, TAK-733, PD318088.

In one embodiment, the bioactive agent is a Raf inhibitor. Raf inhibitors are known and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzene sulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), Sorafenib N-Oxide (4-[4-[[[[4-Chloro-3 (trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, SB590885, ZM336372, GW5074, TAK-632, CEP-32496, LY3009120, and GX818 (Encorafenib).

In one embodiment, the bioactive agent is an AKT inhibitor, including but not limited to, MK-2206, GSK690693, Perifosine, (KRX-0401), GDC-0068, Triciribine, AZD5363, Honokiol, PF-04691502, and Miltefosine, a FLT-3 inhibitor, including but not limited to, P406, Dovitinib, Quizartinib (AC220), Amuvatinib (MP-470), Tandutinib (MLN518), ENMD-2076, and KW-2449, or a combination thereof.

In one embodiment, the bioactive agent is an mTOR inhibitor. Examples of mTOR inhibitors include but are not limited to rapamycin and its analogs, everolimus (Afinitor), temsirolimus, ridaforolimus, sirolimus, and deforolimus. Examples of MEK inhibitors include but are not limited to tametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2 S)-piperidin-2-yl]azetidin-3-ol) (cobimetinib), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H, 8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide).

In one embodiment, the bioactive agent is a RAS inhibitor. Examples of RAS inhibitors include but are not limited to Reolysin and siG12D LODER.

In one embodiment, the bioactive agent is a HSP inhibitor. HSP inhibitors include but are not limited to Geldanamycin or 17-N-Allylamino-17-demethoxygeldanamycin (17AAG), and Radicicol.

Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhbitor, a c-MET inhibitor, a PARP inhibitor, a Cdk inhibitor, an IGFR-TK inhibitor, an anti-HGF antibody, a focal adhesion kinase inhibitor, a Map kinase kinase (mek) inhibitor, a VEGF trap antibody, pemetrexed, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In one embodiment, the bioactive agent is selected from, but are not limited to, Imatinib mesylate (Gleevac®), Dasatinib (Sprycel®), Nilotinib (Tasigna®), Bosutinib (Bosulif®), Trastuzumab (Herceptin®), trastuzumab-DM1, Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Tagretin®), Alitretinoin (Panretin®), Tretinoin (Vesanoid®), Carfilizomib (Kyprolis™), Pralatrexate (Folotyn®), B evacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

In certain aspects, the bioactive agent is an anti-inflammatory agent, a chemotherapeutic agent, a radiotherapeutic, an additional therapeutic agent, or an immunosuppressive agent.

Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin®) or liposomal vincristine (Marqibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents that can be administered in combination with a Degrader disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991, ribociclib (LEE011), amebaciclib (LY2835219), HDM201, fulvestrant (Faslodex), exemestane (Aromasin), PIA/1447, ruxolitinib (INC424), BGJ398, necitumumab, pemetrexed (Alimta), and ramucirumab (IMC-1121B).

In one embodiment, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

In one aspect of the present invention, the bioactive agent is an immunosuppressive agent. The immunosuppressive agent can be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g.ridaforolimus, azathioprine, campath 1H, a S1P receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3A, 33B3.1, deoxyspergualin, tresperimus, Leflunomide ARAVA®, CTLAI-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA41 g (Abatacept), belatacept, LFA31 g-etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

### Pharmaceutical Compositions and Dosage Forms

In some aspects, this invention is a pharmaceutical composition comprising a therapeutically effective amount of a BRD9-binding compound of Formula I, II, III, or IV or a MTH1-binding compound of Formula V or VI as described herein, and one or more pharmaceutically acceptable carriers such as a diluent, preservative, solubilizer, emulsifier, adjuvant, excipient, gel, or solidification material. Such excipients include but are not limited to liquids such as water, saline, glycerol, polyethylene glycol, hyaluronic acid, ethanol, and the like. The compound can be provided, for example, in the form of a solid, a liquid, spray dried material, a microparticle, nanoparticle, controlled release system, etc., as desired according to the goal of the therapy.

The term “pharmaceutically acceptable carrier” refers to a diluent, adjuvant, excipient or carrier with which a compound of the disclosure is administered. The terms “effective amount” or “pharmaceutically effective amount” refer to a sufficient amount of the agent to provide the desired biological result. That result can be reduction and/or alleviation of the signs, symptoms, or causes of the target disorder that is mediated by an estrogen receptor. An appropriate “effective” amount in any individual, for example a human, case can be determined by the healthcare provider based on the needs of the patient. “Pharmaceutically acceptable carriers” for therapeutic use are well known in the pharmaceutical art, and are described, for example, in Remington's Pharmaceutical Sciences, 18th Edition (Easton, Pa.: Mack Publishing Company, 1990). For example, sterile saline and phosphate-buffered saline at physiological pH can be used. Preservatives, stabilizers, dyes and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

Suitable excipients for non-liquid formulations are also known to those of skill in the art. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the composition of the disclosure can be administered as a pharmaceutical formulation including one suitable for oral (including buccal and sub-lingual), rectal, nasal, topical, transdermal, pulmonary, vaginal or parenteral (including intramuscular, intra-arterial, intrathecal, subcutaneous and intravenous) administration or in a form suitable for administration by inhalation or insufflation. A typical manner of administration is oral, topical or intravenous, using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment provided is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are typical oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Typically, sterile injectable suspensions are formulated according to techniques known in the art using suitable carriers, dispersing or wetting agents and suspending agents. The sterile injectable formulation can also be a sterile injectable solution or a suspension in a acceptably nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as an continuous infusion system. A formulation provided by the disclosure can be administered using a syringe, injector, pump, or any other device recognized in the art for parenteral administration.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, typical methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Typical formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art. The compounds of the disclosure can also be delivered through the skin or mucosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound may, for example generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. the effective amount for a given situation can be determined by routine experimentation. For purposes of the disclosure, a therapeutic amount may for example be in the range of about 0.01 mg/kg to about 250 mg/kg body weight, more typically about 0.1 mg/kg to about 10 mg/kg, in at least one dose. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

In certain embodiments the pharmaceutical composition is in a dosage form that contains from about 0.1 mg to about 2000 mg, from about 10 mg to about 1000 mg, from about 50 mg to about 600 mg, or from about 100 mg to about 400 mg of the active compound. In another embodiment the pharmaceutical composition is in a dosage form that contains from about 0.1 mg to about 100 mg, from about 0.5 mg to about 100 mg, from about 1 mg to about 50 mg, or from about 2 mg to about 25 mg of the active compound. In another embodiment the pharmaceutical composition is in a dosage form that contains from about 0.1 mg to about 10 mg, from about 0.5 mg to about 8 mg, from about 0.5 mg to about 6 mg, or from about 0.5 mg to about 5 mg of the active compound. Examples are dosage forms with at least, or in some embodiments, not more than, 0.1, 1, 5, 10, 25, 50, 100, 200, 250, 300, 400, 500, 600, 700, or 750 mg of active compound, or its salt.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### General Synthesis

The compounds described herein can be prepared by methods known by those skilled in the art. In one non-limiting example, the disclosed compounds can be made using the schemes below.

Compounds of the present invention with stereocenters may be drawn without stereochemistry for convenience. One skilled in the art will recognize that pure or enriched enantiomers and diastereomers can be prepared by methods known in the art. Examples of methods to obtain optically active materials include at least the following:
a. physical separation of crystals—a technique whereby macroscopic crystals of the individual enantiomers are manually separated. This technique can be used if crystals of the separate enantiomers exist, i.e., the material is a conglomerate, and the crystals are visually distinct;
b. simultaneous crystallization—a technique whereby the individual enantiomers are separately crystallized from a solution of the racemate, possible only if the enantiomer is a conglomerate in the solid state;
c. enzymatic resolutions—a technique whereby partial or complete separation of a racemate by virtue of differing rates of reaction for the enantiomers with an enzyme;
d. enzymatic asymmetric synthesis—a synthetic technique whereby at least one step in the synthesis uses an enzymatic reaction to obtain an enantiomerically pure or enriched synthetic precursor of the desired enantiomer;
e. chemical asymmetric synthesis—a synthetic technique whereby the desired enantiomer is synthesized from an achiral precursor under conditions that produce asymmetry (i.e. chirality) in the product, which may be achieved by chiral catalysts or chiral auxiliaries;
f. diastereomer separations—a technique whereby a racemic compound is reaction with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer;
g. first- and second-order asymmetric transformations—a technique whereby diastereomers from the racemate quickly equilibrate to yield a preponderance in solution of the diastereomer from the desired enantiomer of where preferential crystallization of the diastereomer from the desired enantiomer perturbs the equilibrium such that eventually in principle all the material is converted to the crystalline diastereomer from the desired enantiomers. The desired enantiomer is then released from the diastereomer;
h. kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
i. enantiospecific synthesis from non-racemic precursors—a synthetic technique whereby the desired enantiomer is obtained from non-chiral starting materials and where the stereochemical integrity is not or is only minimally compromised over the course of the synthesis;
j. chiral liquid chromatography—a technique whereby the enantiomers of a racemate are separated in a liquid mobile phase by virtue of their differing interactions with a stationary phase (including vial chiral HPLC). The stationary phase can be made of chiral material or the mobile phase can contain an additional chiral material to provoke the differing interactions;
k. chiral gas chromatography—a technique whereby the racemate is volatilized and enantiomers are separated by virtue of their differing interactions in the gaseous mobile phase with a column containing a fixed non-racemic chiral adsorbent phase;
l. extraction with chiral solvents—a technique whereby the enantiomers are separated by virtue of preferential dissolution of one enantiomer into a particular chiral solvent;
m. transport across chiral membranes—a technique whereby a racemate is place in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through;
n. simulated moving bed chromatography is used in one embodiment. A wide variety of chiral stationary phases are commercially available.

### Example 1. Synthesis of BRD9 Degraders

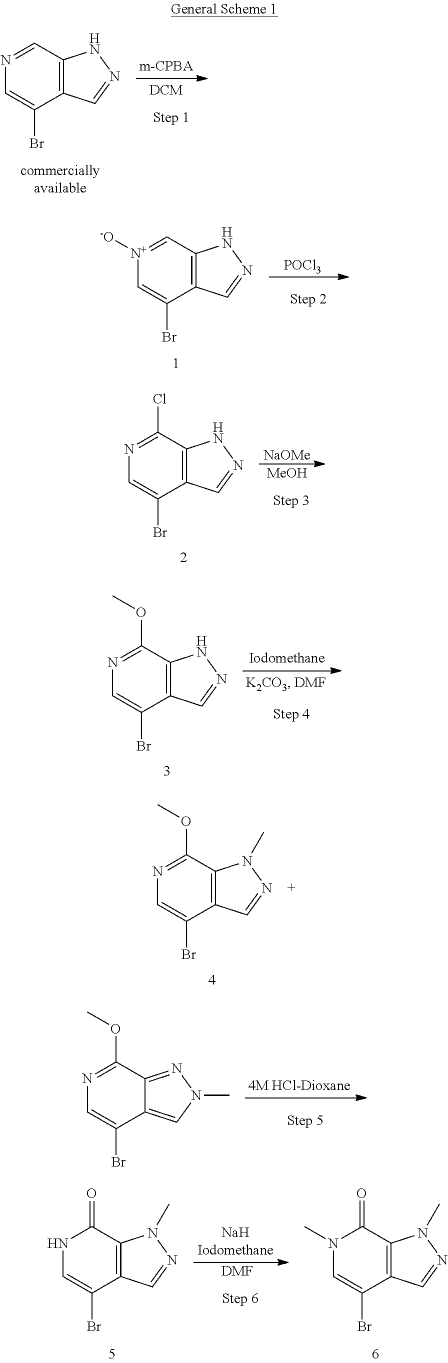

Step 1: 4-Bromo-1H-pyrazolo[3,4-c]pyridine (2 g, 10.10 mmol) was dissolved in DCM (30 mL) and m-CPBA (2.61 g, 15.15 mmol) was added at 0° C. The reaction mixture was stirred at room temperature for 16 hours. After completion of the reaction, solid precipitate was filtered and dried to afford 4-bromo-6-oxido-1H-pyrazolo[3,4-c]pyridin-6-ium (1, 2 g, 9.34 mmol, 92.52% yield). LCMS (ES+): m/z 215 [M+H]+

Step 2: 4-Bromo-6-oxido-1H-pyrazolo[3,4-c]pyridin-6-ium (1, 2 g, 9.34 mmol) was taken up in a 25 ml round bottom flask. To the flask was added POCl₃ (1.43 g, 9.34 mmol, 5 mL) at 0° C. and the reaction was stirred for 16 hours at room temperature. After completion, the POCl₃ was evaporated under reduced pressure and the reaction was quenched with ice and allowed to stir for 10 minutes.

The solid precipitate was filtered and dried to obtain 4-bromo-7-chloro-1H-pyrazolo[3,4-c]pyridine (2, 1.7 g, 7.31 mmol, 78.26% yield). LCMS (ES+): m/z 233 [M+H]+

Step 3: To 4-bromo-7-chloro-1H-pyrazolo[3,4-c]pyridine (2, 1.7 g, 7.31 mmol) and methanol (10 mL) in a 20 ml sealed tube was added sodium methoxide solution (1.98 g, 36.56 mmol, 2.04 mL). The reaction was stirred at 80° C. for 16 hours. After completion of the reaction, methanol was evaporated under reduced pressure and the resulting residue was extracted with EtOAc, dried over anhydrous sodium sulfate, filtered and evaporated to dryness to obtain 4-bromo-7-methoxy-1H-pyrazolo[3,4-c]pyridine (3, 1.5 g, 6.58 mmol, 89.95% yield) as an off-white colored solid. LCMS (ES+): m/z 229 [M+H]+

Step 4: 4-Bromo-7-methoxy-1H-pyrazolo[3,4-c]pyridine (3, 1 g, 4.39 mmol) was taken up in DMF (15 mL) and potassium carbonate (anhydrous, 99% (1.82 g, 13.16 mmol, 793.97 uL) was added. The reaction was stirred at 0° C. before methyl iodide (746.90 mg, 5.26 mmol, 327.59 uL) was added drop-wise. The reaction was stirred at room temperature for 16 hours. After completion, the reaction was extracted with EtOAc twice. The combined organic layers were dried over anhydrous Na₂SO₄ and excess solvent was removed under reduced pressure. The crude material was purified by silica gel chromatography (30% EA/Pet ether) to obtain 4-bromo-7-methoxy-1-methyl-pyrazolo[3,4-c]pyridine (4, 0.5 g, 2.07 mmol, 47.10% yield) as an off-white solid. LCMS (ES+): m/z 243 [M+H]+

Step 5: To a stirred solution of 4-bromo-7-methoxy-1-methyl-pyrazolo[3,4-c]pyridine (4, 0.45 g, 1.86 mmol) in dioxane (5 mL) was added 4M dioxane-HCl (1.86 mmol, 5 mL) and the reaction was stirred at 50° C. for 16 hours. After completion of the reaction, excess solvent was removed under reduced pressure to afford 4-bromo-1-methyl-6H-pyrazolo[3,4-c]pyridin-7-one (5, 0.4 g, 1.75 mmol, 94.36% yield) as an off-white colored solid. LCMS (ES+): m/z 229 [M+H]+

Step 6: 4-Bromo-1-methyl-6H-pyrazolo[3,4-c]pyridin-7-one (5, 200 mg, 877.02 μmol) was taken up in DMF (10 mL) and sodium hydride (60% dispersion in mineral oil (40.32 mg, 1.75 mmol)) was added. The reaction was brought at 0° C. and methyl iodide (186.72 mg, 1.32 mmol, 81.90 uL) was added drop-wise. The reaction was allowed to stir at room temperature for 16 hours. After completion, the reaction was extracted with EtOAc twice and the combined organic layers were dried over anhydrous Na₂SO₄. Excess solvent was removed under reduced pressure to obtain 4-bromo-1,6-dimethyl-pyrazolo[3,4-c]pyridin-7-one (6, 150 mg, 619.65 μmol, 70.65% yield) as an off-white colored solid. LCMS (ES+): m/z 243 [M+H]+

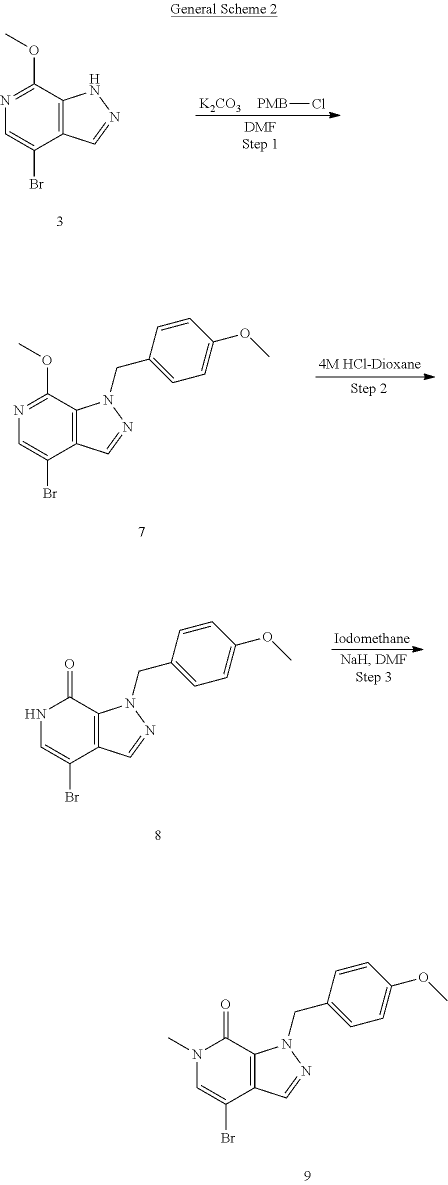

Step 1: 4-Bromo-7-methoxy-1H-pyrazolo[3,4-c]pyridine (3, 0.5 g, 2.19 mmol) was taken up in DMF (5 mL) and anhydrous potassium carbonate was added (909.07 mg, 6.58 mmol, 396.97 uL). The reaction was stirred at 0° C. before 4-methoxy benzyl bromide (661.25 mg, 3.29 mmol) was added drop-wise. The reaction mixture and stirred at room temperature for 16 hours. After completion, the reaction was extracted with EtOAc twice and the combined organic layers were dried over anhydrous Na₂SO₄. Excess solvent was removed under reduced pressure to obtain 4-bromo-7-methoxy-1-[(4-methoxyphenyl)methyl]pyrazolo[3,4-c]pyridine (7, 0.6 g, crude) as a mixture of regioisomers. LCMS (ES+): m/z 349 [M+H]+

Step 2: To a stirred solution of 4-bromo-7-methoxy-1-[(4-methoxyphenyl)methyl]pyrazolo[3,4-c]pyridine (7, 0.6 g, 1.72 mmol) in dioxane (5 mL) was added 4M dioxane-HCl (2.07 mmol, 5 mL). The reaction was stirred at 50° C. for 16 hours. After completion, excess solvent was evaporated under reduced pressure to afford 4-bromo-1-[(4-methoxyphenyl)methyl]-6H-pyrazolo[3,4-c]pyridin-7-one (8, 0.5 g, crude) as an off-white colored solid. LCMS (ES+): m/z 335 [M+H]+

Step 3: 4-Bromo-1-[(4-methoxyphenyl)methyl]-6H-pyrazolo[3,4-c]pyridin-7-one (8, 0.5 g, 1.50 mmol) was taken up in DMF (10 mL) and sodium hydride (60% dispersion in mineral oil (51.60 mg, 2.24 mmol)) was added. The reaction was brought to 0° C. before methyl iodide (318.57 mg, 2.24 mmol, 139.72 uL) was added drop-wise and the reaction mixture and was stirred at room temperature for 16 hours. After completion, the reaction was extracted with EtOAc and the combined organic layers were dried over anhydrous Na₂SO₄. Excess solvent was removed under reduced pressure to obtain 4-bromo-1-[(4-methoxyphenyl)methyl]-6-methyl-pyrazolo[3,4-c]pyridin-7-one (9, 0.6 g, crude) as an off-white colored solid. LCMS (ES+): m/z 349 [M+H]⁺

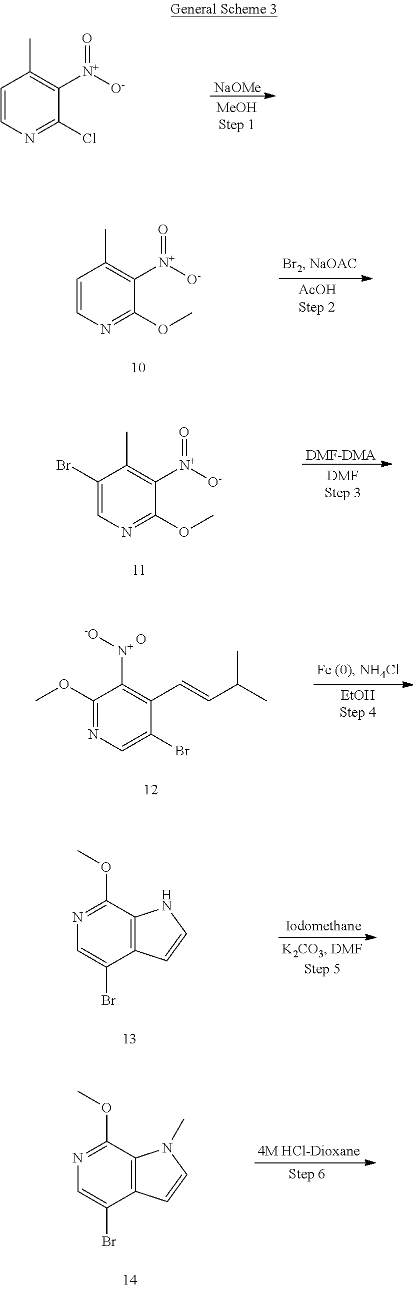

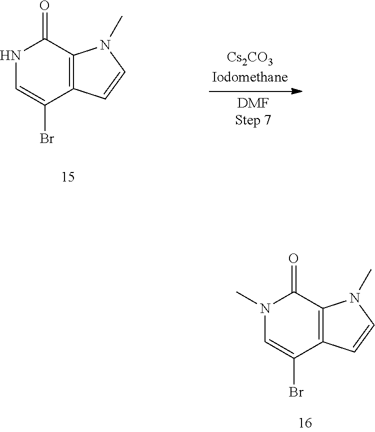

Step 1: To a stirred solution of 2-chloro-4-methyl-3-nitro-pyridine (50 g, 289.74 mmol) in methanol (500 mL) was added sodium methoxide (46.96 g, 869.22 mmol, 48.46 mL) portion-wise. The reaction was stirred at 80° C. for 6 hours. The progress of the reaction was monitored by TLC and LC-MS. The reaction mixture was quenched with water 500 mL and the resultant solid was filtered to afford 2-methoxy-4-methyl-3-nitro-pyridine (10, 44 g, 261.67 mmol, 90.31% yield). LCMS (ES+): m/z 169 [M+H]+

Step 2: To a stirred solution of 2-methoxy-4-methyl-3-nitro-pyridine (10, 44 g, 261.67 mmol) and sodium acetate (anhydrous, 77.28 g, 942.02 mmol, 50.51 mL) in acetic acid (400 mL) was added bromine (112.91 g, 706.51 mmol). The reaction mixture was diluted with saturated sodium sulfate solution (500 mL). The resultant solid was filtered and the solid was washed with water (2 L) to remove sodium sulfate. The filtered solid was dried to afford 5-bromo-2-methoxy-4-methyl-3-nitro-pyridine (11, 54 g, 218.58 mmol, 83.53% yield). LCMS (ES+): m/z 248 [M+H]+

Step 3: To a stirred solution of 5-bromo-2-methoxy-4-methyl-3-nitro-pyridine (11, 64.64 g, 261.67 mmol) in DMF (1.2 L), N,N-Dimethylformamide dimethyl acetal (300 g, 2.52 mol, 337.08 mL) was added drop-wise at 80° C. and the reaction was stirred at 95° C. for 12 hours. The progress of the reaction was monitored by TLC. The reaction mixture was diluted with water (2 L) and the precipitated solid was filtered and dried to afford (E)-2-(5-bromo-2-methoxy-3-nitro-4-pyridyl)-N,N-dimethyl-ethenamine (12, 61 g, 201.90 mmol, 77.16% yield). LCMS (ES+): m/z 303 [M+H]+

Step 4: To a stirred solution of (E)-2-(5-bromo-2-methoxy-3-nitro-4-pyridyl)-N,N-dimethyl-ethenamine (12, 61 g, 201.90 mmol) in methanol (1.2 L) and water (300 mL) was added iron powder (61 g, 1.09 mol, 7.76 mL) followed by ammonium chloride (61 g, 1.14 mol, 39.87 mL). The reaction mixture was stirred at 80° C. for 20 hours. The progress of the reaction was monitored by LC-MS and TLC. The hot reaction mixture was filtered through a pad of Celite® and the filtrate was concentrated under reduced pressure. The resultant residue was extracted with ethyl acetate (1 L) and water. The crude product was purified by column chromatography to afford 4-bromo-7-methoxy-1H-pyrrolo[2,3-c]pyridine (13, 21 g, 92.49 mmol, 45.81% yield). LCMS (ES+): m/z 228 [M+H]+

Step 5: To a stirred solution of 4-bromo-7-methoxy-1H-pyrrolo[2,3-c]pyridine (13, 21 g, 92.49 mmol) in N,N-dimethylformamide (400 mL) was added anhydrous potassium carbonate (38.35 g, 277.46 mmol, 16.75 mL) followed by iodomethane (65.64 g, 462.44 mmol, 28.79 mL) drop-wise at 0° C. The reaction mixture was stirred for 5 hours. The progress of the reaction was monitored by TLC and LC-MS. The reaction mixture was diluted with water and the precipitated solid was filtered and dried to afford 4-bromo-7-methoxy-1-methyl-pyrrolo[2,3-c]pyridine (14, 20 g, 82.96 mmol, 89.70% yield). LCMS (ES+): m/z 242 [M+H]+

Step 6: Into a 250 mL sealed tube containing a well-stirred solution of 4-bromo-7-methoxy-1-methyl-pyrrolo[2,3-c]pyridine (14, 1.8 g, 7.47 mmol) in ethanol (10 mL) was added 48% HBr aqueous solution (7.47 mmol, 30 mL) and the reaction was heated at 90° C. for 2 hours. TLC indicated complete consumption of starting material. Ice cold water (50 mL) was added and the reaction was stirred for 10 minutes. The resulting solid was filtered and dried to afford 4-bromo-1-methyl-6H-pyrrolo[2,3-c]pyridin-7-one (15, 1.4 g, 6.17 mmol, 82.58% yield) as a brown solid. LCMS (ES+): m/z 228 [M+H]+

Step 7: Into a 100 mL two-necked round-bottomed flask containing a well-stirred solution of 4-bromo-1-methyl-6H-pyrrolo[2,3-c]pyridin-7-one (15, 1.4 g, 6.17 mmol) in anhydrous THF (20 mL) were added cesium carbonate (4.02 g, 12.33 mmol) and methyl iodide (1.75 g, 12.33 mmol, 767.70 uL) under nitrogen atmosphere at room temperature. The resulting mixture was stirred at room temperature for 2 hours. TLC indicated complete consumption of starting material. To the crude mass was added water (50 mL) and the aqueous phase was extracted twice with EtOAc (2×75 mL). The combined organic phases were dried over anhydrous sodium sulfate, filtered and concentrated under reduced pressure to afford 1.4 g of 4-bromo-1,6-dimethyl-pyrrolo[2,3-c]pyridin-7-one (16, 1.4 g, 5.81 mmol, 94.18% yield) as an off white solid. LCMS (ES+): m/z 242 [M+H]+

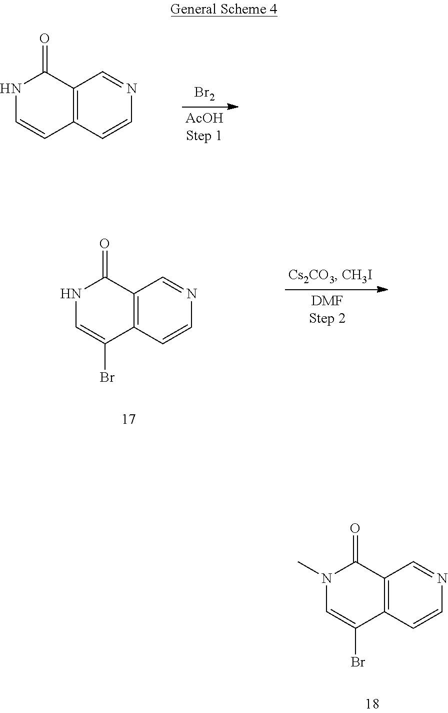

Step 1: Into a 100 mL single-necked round-bottomed flask containing a well-stirred solution of 2H-2,7-naphthyridin-1-one (1.7 g, 11.63 mmol) in acetic acid (20 mL) was added bromine (1.86 g, 11.63 mmol) under nitrogen atmosphere at room temperature. The resulting mixture was heated at 50° C. for 1 hour at which point TLC indicated complete consumption of starting material. Ice cold water (50 mL) was added and the reaction was stirred for 10 minutes. The resulting solid was filtered and dried to afford crude material that was purified by preparative HPLC to afford 4-bromo-2H-2,7-naphthyridin-1-one (17, 720 mg, 3.20 mmol, 27.50% yield) as an off-white solid. LCMS (ES+): m/z 226 [M+H]+

Step 2: Into a 50 mL two-necked round-bottomed flask containing a well-stirred solution of 4-bromo-2H-2,7-naphthyridin-1-one (17, 0.72 g, 3.20 mmol) in anhydrous THF (20 mL) were added cesium carbonate (2.08 g, 6.40 mmol) and methyl iodide (681.18 mg, 4.80 mmol, 298.76 uL) under nitrogen atmosphere at room temperature. The resulting mixture was stirred at room temperature for 3 hours at which point TLC indicated complete consumption of starting material. To the crude mixture, 50 mL of water was added and the aqueous phase was extracted twice with EtOAc (2×50 mL). The organic layer was dried with anhydrous Na₂SO₄, filtered and concentrated under reduced pressure to afford 530 mg of 4-bromo-2-methyl-2,7-naphthyridin-1-one (18, 530 mg, 2.22 mmol, 69.29% yield) as an off white solid. LCMS (ES+): m/z 226 [M+H]+

### Synthesis of Boronate Esters:

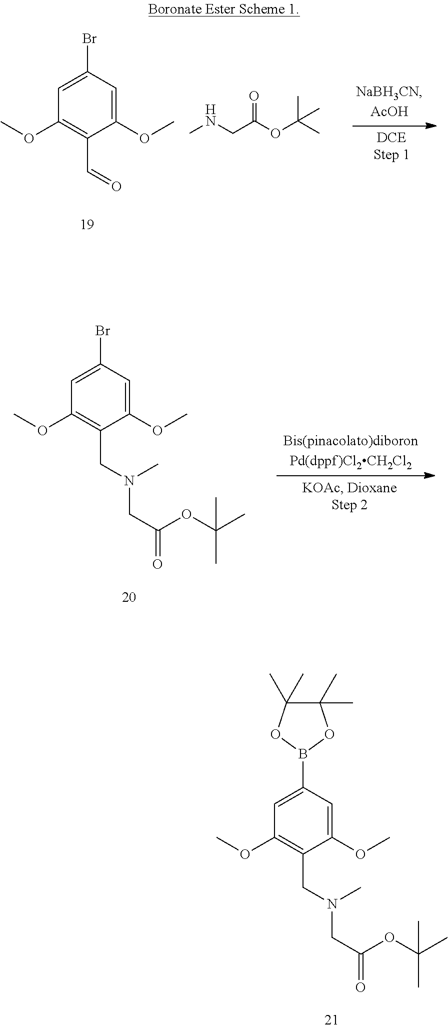

Step 1: To an oven-dried pressure tube charged with a solution of 4-bromo-2,6-dimethoxy-benzaldehyde (19, 2 g, 8.16 mmol) in 1,2-dichloroethane (60 mL), tert-butyl sarcosinate hydrochloride (1.78 g, 9.79 mmol) and acetic acid (490.01 mg, 8.16 mmol, 466.67 uL) were added at room temperature. The reaction mixture was heated to 90° C. for 2 hours. After cooling to 0° C., sodium cyanoborohydride (1.03 g, 16.32 mmol) was added portion-wise and the reaction mixture was warmed to room temperature. The reaction mixture was stirred for 16 hours at room temperature. The reaction mixture was quenched with saturated sodium bicarbonate solution (30 mL) and the product was extracted with dichloromethane (2×80 mL). The organic layer was dried over anhydrous sodium sulfate, filtered and concentrated under reduced pressure. The crude mixture was purified by column chromatography on silica (28% ethyl acetate\pet.ether) to yield tert-butyl 2-[(4-bromo-2,6-dimethoxy-phenyl)methyl-methyl-amino]acetate (20, 2.7 g, 6.45 mmol, 79.05% yield) as colorless oil. LCMS (ES+): m/z 375 [M+H]+

Step 2: An oven dried pressure tube was charged with a solution of tert-butyl 2-[(4-bromo-2,6-dimethoxy-phenyl)methyl-methyl-amino]acetate (20, 1 g, 2.67 mmol) in 1,4-dioxane (10 mL), bis((−)-pinanediolato) diboron (1.24 g, 3.47 mmol) and potassium acetate (655.56 mg, 6.68 mmol, 417.55 uL). The reaction mixture was purged with nitrogen for 5 minutes before Pd(dppf)Cl₂.CH₂Cl₂ (218.19 mg, 267.19 μmol) was added. The reaction mixture was heated to 80° C. for 16 hours and then cooled to room temperature. The reaction was diluted with water (20 mL) and the product was extracted with ethyl acetate (2×60 mL). The combined organic layers were dried over anhydrous sodium sulfate, filtered and concentrated under reduced pressure. The crude mixture was purified by column chromatography on silica (30-60% ethyl acetate\Pet.ether) to yield tert-butyl 2-[[2,6-dimethoxy-4-(4,4,5,5-tetramethyl-1,3,2-dioxaborolan-2-yl)phenyl]methyl-methyl-amino]acetate (21, 700 mg, 977.73 μmol, 36.59% yield). LCMS (ES+): m/z 422 [M+H]+

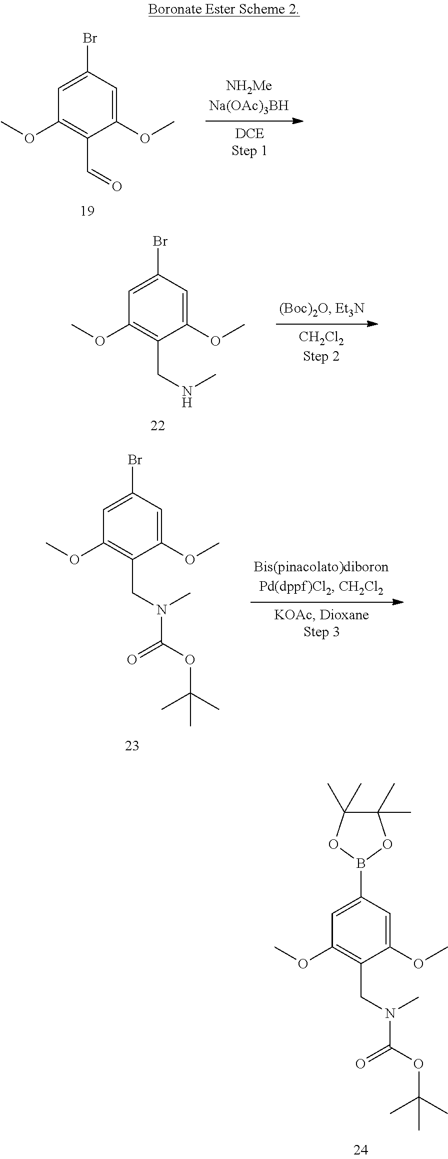

Step 1: Into a 50 mL two-necked round-bottomed flask containing a well-stirred solution of 4-bromo-2,6-dimethoxy-benzaldehyde (19, 3 g, 12.24 mmol) in methanol (15 mL) was added methylamine solution (380.18 mg, 12.24 mmol, 15 mL) under nitrogen atmosphere at room temperature. The resulting mixture was stirred for 20 minutes followed by the addition of sodium borohydride (926.25 mg, 24.48 mmol, 865.65 uL) under a nitrogen atmosphere at room temperature. The resulting mixture was stirred at room temperature for 16 hours. A saturated aqueous sodium bicarbonate solution (25 mL) was added and the solution was extracted with EtOAc (3×50 mL). The combined organic phases were washed with brine, dried with anhydrous sodium sulfate, filtered and concentrated under reduced pressure to afford 1.2 g of 1-(4-bromo-2,6-dimethoxy-phenyl)-N-methyl-methanamine (22, 3.0 g, 11.53 mmol, 94.21% yield) as a colorless liquid. LCMS (ES+): m/z 261 [M+H]+

Step 2: Into a 50 mL two-necked round-bottomed flask containing a well-stirred solution of 1-(4-bromo-2,6-dimethoxy-phenyl)-N-methyl-methanamine (22, 0.5 g, 1.92 mmol) in THF (10 mL) was added Boc anhydride (629.25 mg, 2.88 mmol, 661.67 uL) and TEA (389.00 mg, 3.84 mmol, 535.82 uL) under nitrogen atmosphere at room temperature. The resulting mixture was stirred at room temperature for 4 hours. TLC indicated complete consumption of starting material. Water (25 mL) was added and the aqueous phase was extracted twice with EtOAc (2×50 mL). The combined organic phases were washed with brine, dried over anhydrous sodium sulfate, filtered and concentrated under reduced pressure to afford 0.62 g of tert-butyl N-[(4-bromo-2,6-dimethoxy-phenyl)methyl]-N-methyl-carbamate (23, 620 mg, 1.72 mmol, 89.54% yield) as a colorless liquid. LCMS (ES+): m/z 261 [M+H-Boc]+

Step 3: Into a 50 mL sealed tube containing a mixture of tert-butyl N-[(4-bromo-2,6-dimethoxy-phenyl)methyl]-N-methyl-carbamate (23, 0.65 g, 1.80 mmol) in anhydrous dioxane (12 mL) were added bis((−)-pinanediolato) diboron (775.34 mg, 2.17 mmol) and potassium acetate (354.16 mg, 3.61 mmol, 225.58 uL). Argon gas was bubbled through the reaction mixture for 10 minutes followed by addition of Pd(dppf)Cl₂.CH₂Cl₂ (147.35 mg, 180.43 μmol). The resulting suspension was purged with argon gas for an additional 10 minutes. The reaction was stirred at 90° C. for 3 hours. The reaction mixture was passed through a pad of Celite®, the filtrate was concentrated under reduced pressure, and purified by flash silica-gel (230-400 mesh) with 2:8 EtOAc/petroleum ether to afford tert-butyl N-[[2,6-dimethoxy-4-(4,4,5,5-tetramethyl-1,3,2-dioxaborolan-2-yl)phenyl]methyl]-N-methyl-carbamate (24, 0.5 g, 1.23 mmol, 68.03% yield). LCMS (ES+): m/z 308 [M+H-Boc]+

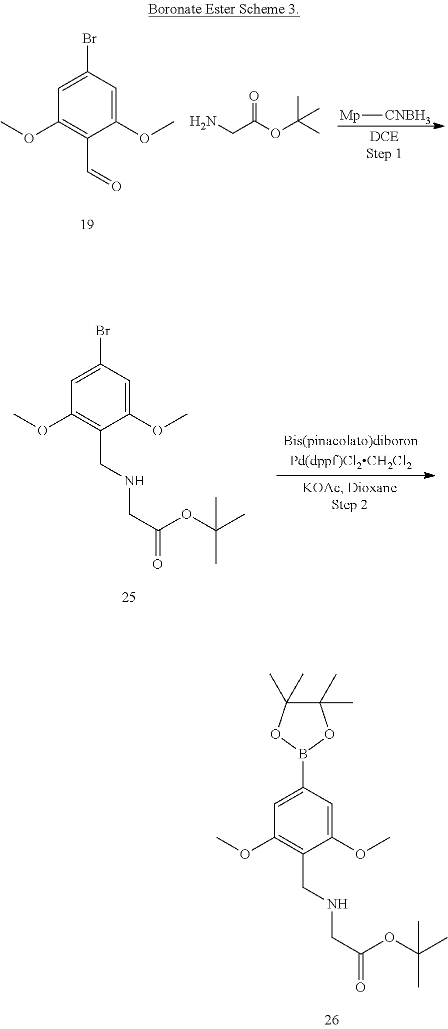

Step 1: Into a 250 ml RBF 4-bromo-2,6-dimethoxy-benzaldehyde (19, 5 g, 20.40 mmol) and 4-bromo-2,6-dimethoxy-benzaldehyde (5 g, 20.40 mmol) were taken up in Methanol (100 mL) and stirred at RT for 1 h. After that MP-CNBH3 (7.5 g, 20.40 mmol) was added to the reaction mixture and allowed to stir for 16 hours. Upon completion, the reaction was filtered through a pad of Celite® and washed with methanol. The mother liquor was concentrated to afford crude compound that was purified by silica gel chromatography eluting with 2% MeOH/DCM to obtain tert-butyl 2-[(4-bromo-2,6-dimethoxy-phenyl)methylamino]acetate (25, 2 g, 5.55 mmol, 27.21% yield). LCMS (ES+): m/z 361 [M+H]+

Step 2: Into a 100 mL round bottom flask containing a mixture of tert-butyl 2-[(4-bromo-2,6-dimethoxy-phenyl)methylamino]acetate (25, 2 g, 5.55 mmol) in anhydrous dioxane (20 mL) were added 4,4,5,5-tetramethyl-2-(4,4,5,5-tetramethyl-1,3,2-dioxaborolan-2-yl)-1,3,2-dioxaborolane (1.69 g, 6.66 mmol) and potassium acetate (1.09 g, 11.10 mmol, 694.08 uL). Argon gas was bubbled through the reaction mixture for 10 minutes and followed by the addition of 1,1′-bis(diphenylphosphino)ferrocene]palladium(II) dichloride (406.23 mg, 555.18 μmol). The resulting suspension was purged with argon gas for an additional 10 minutes and then stirred at 100° C. for 8 hours. The reaction mixture was passed through a pad of Celite® and the mother liquor was concentrated under reduced pressure and purified by preparative HPLC to afford tert-butyl (2,6-dimethoxy-4-(4,4,5,5-tetramethyl-1,3,2-dioxaborolan-2-yl)benzyl)glycinate (26, 1.1 g, crude). LCMS (ES+): m/z 407 [M+H]+

### Final Targeting Ligand Synthesis (N-Me Pyrazole)

### Targeting Ligand Synthesis 1

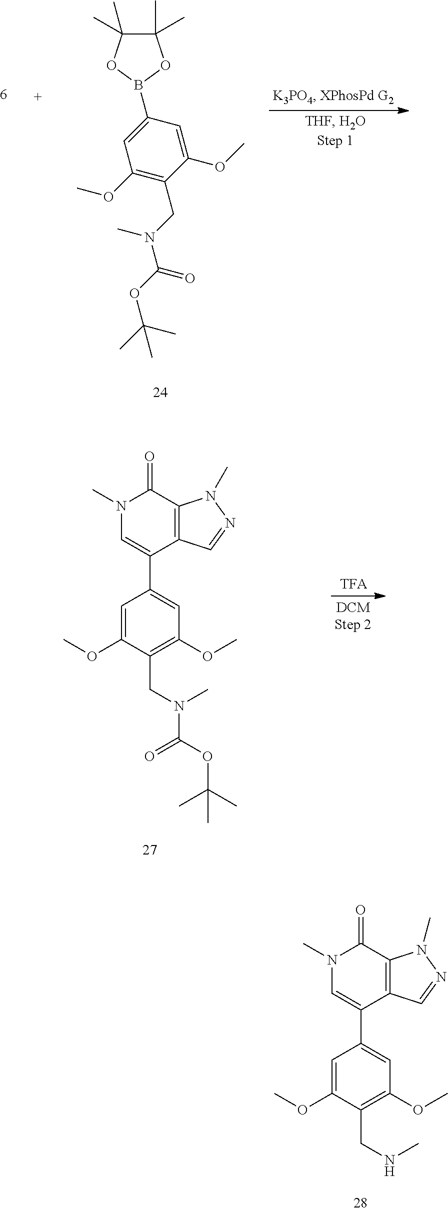

Step 1: Into a 10 mL sealed tube containing a mixture of 4-bromo-1,6-dimethyl-pyrazolo[3,4-c]pyridin-7-one (6, 50 mg, 206.55 μmol) and tert-butyl N-[[2,6-dimethoxy-4-(4,4,5,5-tetramethyl-1,3,2-dioxaborolan-2-yl)phenyl]methyl]-N-methyl-carbamate (24, 84.13 mg, 206.55 μmol) in THF (4 mL) and water (1 mL) was added potassium phosphate tribasic (87.69 mg, 413.10 μmol). Argon gas was bubbled through the reaction mixture for 5 minutes followed by the addition of XPhos-Pd-G2 (9.75 mg, 6.20 μmol). The resulting suspension was purged with argon gas for an additional 5 minutes. The contents were heated at 100° C. for 5 hours. After completion, the reaction was filtered through a pad of Celite® and the filtrate was extracted with EtOAc. The organic layers were combined and solvent was removed under reduced pressure to afford tert-butyl N-[[4-(1,6-dimethyl-7-oxo-pyrazolo[3,4-c]pyridin-4-yl)-2,6-dimethoxy-phenyl]methyl]-N-methyl-carbamate (27, 100 mg, crude). LCMS (ES+): m/z 443 [M+H]+

Step 2: tert-Butyl N-[[4-(1,6-dimethyl-7-oxo-pyrazolo[3,4-c]pyridin-4-yl)-2,6-dimethoxy-phenyl]methyl]-N-methyl-carbamate (27, 100 mg, 225.99 μmol) was dissolved in DCM (5 mL) and TFA (1.48 g, 12.98 mmol, 1 mL) was added at 0° C. The reaction was warmed to room temperature and stirred for 1 hour. After completion, the volatiles were evaporated under reduced pressure. The crude material was purified by preparatory reverse phase purification (SUNFIRE OBD C18(100×30)MM 5μ) Mobile phase: A: 0.1% TFA in water B: ACN) to obtain 4-[3,5-dimethoxy-4-(methylaminomethyl)phenyl]-1,6-dimethyl-pyrazolo[3,4-c]pyridin-7-one (28, 20 mg, 58.41 μmol, 25.85% yield). LCMS (ES+): m/z 343 [M+H]+

### Targeting Ligand Synthesis 2

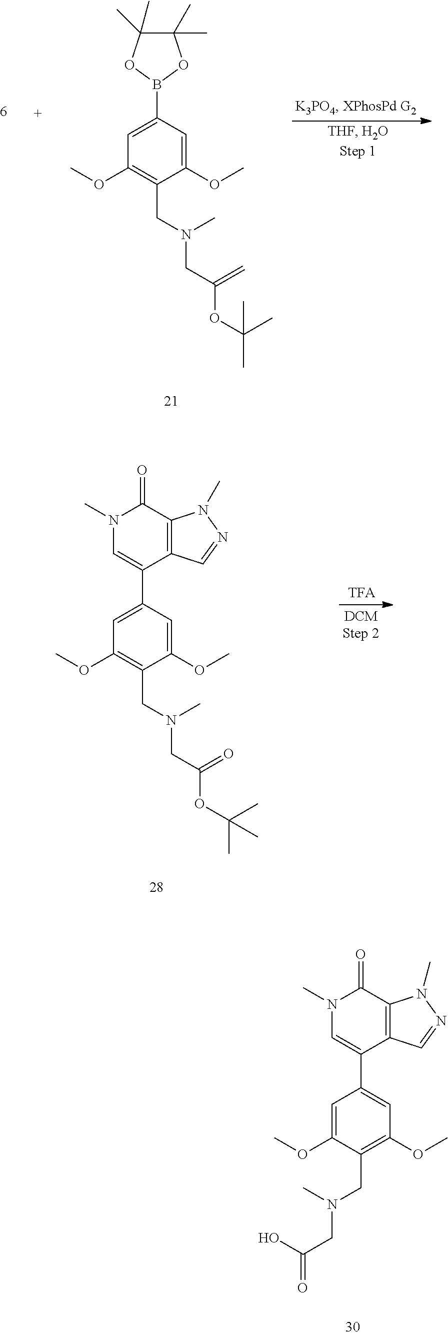

Step 1: Into a 20 mL sealed tube containing a mixture of tert-butyl N-(2,6-dimethoxy-4-(4,4,5,5-tetramethyl-1,3,2-dioxaborolan-2-yl)benzyl)-N-methylglycinate (21, 174.05 mg, 413.10 μmol) and 4-bromo-1,6-dimethyl-pyrazolo[3,4-c]pyridin-7-one (6, 100 mg, 413.10 μmol) in THF (5 mL) and water (1 mL) was added potassium phosphate tribasic (175.38 mg, 826.20 μmol). Argon gas was bubbled through the reaction mixture for 5 minutes, followed by the addition of XPhos-Pd-G2 (9.75 mg, 12.39 μmol). The resulting suspension was purged with argon gas for an additional 5 minutes. The contents were heated at 80° C. for 2 hours. After completion, the reaction was filtered through a pad of Celite® bed and the filtrate was extracted with EtOAc. The solvent was removed under reduced pressure to afford tert-butyl 2-[[4-(1,6-dimethyl-7-oxo-pyrazolo[3,4-c]pyridin-4-yl)-2,6-dimethoxy-phenyl]methyl-methyl-amino]acetate (29, 200 mg, crude). The crude material was used without further purification. LCMS (ES+): m/z 457 [M+H]+

Step 2: tert-Butyl 2-[[4-(1,6-dimethyl-7-oxo-pyrazolo[3,4-c]pyridin-4-yl)-2,6-dimethoxy-phenyl]methyl-methyl-amino]acetate (29, 200 mg, 438.08 μmol) was dissolved in DCM (5 mL) and TFA (2.96 g, 25.96 mmol, 2 mL) was added at 0° C. The reaction mixture was stirred at room temperature for 2 hours. After completion, the volatiles were evaporated under reduced pressure to afford 2-[[4-(1,6-dimethyl-7-oxo-pyrazolo[3,4-c]pyridin-4-yl)-2,6-dimethoxy-phenyl]methyl-methyl-amino]acetic acid (30, 90 mg, 224.76 μmol, 51.31% yield). LCMS (ES+): m/z 401 [M+H]+

### Targeting Ligand Synthesis 3

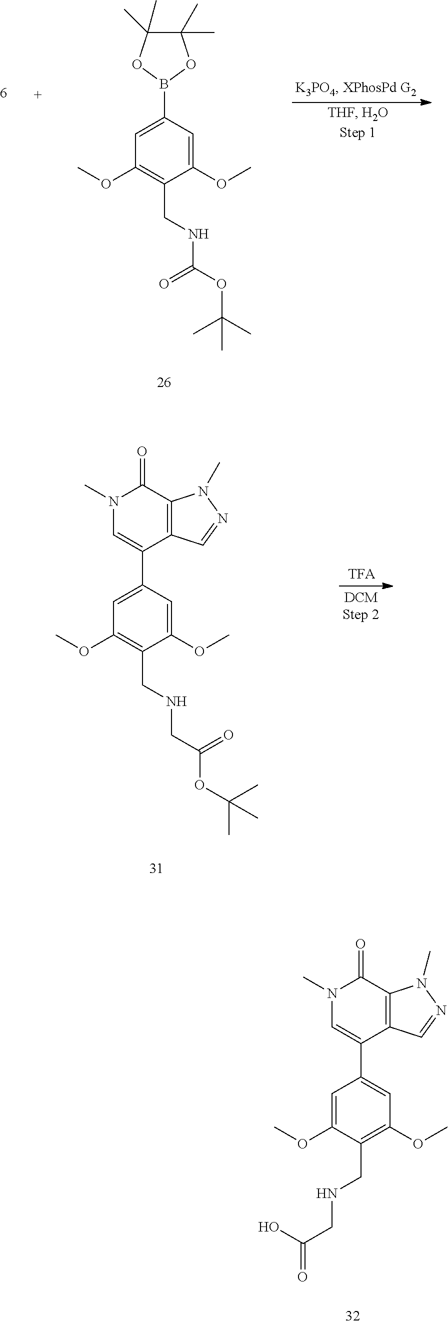

Step 1: Into a 20 mL sealed tube containing a mixture of 4-bromo-1,6-dimethyl-pyrazolo[3,4-c]pyridin-7-one (6, 250 mg, 1.03 mmol) and tert-butyl (2,6-dimethoxy-4-(4,4,5,5-tetramethyl-1,3,2-dioxaborolan-2-yl)benzyl)carbamate (26, 546.84 mg, 1.34 mmol) in water (1 mL) and THF (5 mL) was added potassium phosphate tribasic (438.44 mg, 2.07 mmol). Argon gas was bubbled through the reaction mixture for 10 minutes followed by addition of XPhos-Pd-G2 (162.51 mg, 103.28 μmol). The resulting suspension was purged with argon gas for an additional 10 minutes. The contents were stirred at 80° C. for 2 hours. The reaction mixture was filtered through a pad of Celite® and the filtrate was concentrated under reduced pressure. The reaction mixture was purified by flash silica-gel (230-400 mesh) column with 1:9 MeOH/DCM to afford tert-butyl (4-(1,6-dimethyl-7-oxo-6,7-dihydro-1H-pyrazolo[3,4-c]pyridin-4-yl)-2,6-dimethoxybenzyl)glycinate (31, 350 mg, 790.95 μmol, 76.59% yield) as a brown solid. LCMS (ES+): m/z 443 [M+H]+

Step 2: Into a 50 mL single-necked round-bottomed flask containing a well-stirred solution of (4-(1,6-dimethyl-7-oxo-6,7-dihydro-1H-pyrazolo[3,4-c]pyridin-4-yl)-2,6-dimethoxybenzyl)glycinate (31, 339.25 mg, 766.65 μmol) in anhydrous DCM (8 mL) was added TFA (437.07 mg, 3.83 mmol, 295.31 uL) under nitrogen atmosphere at room temperature. The resulting mixture was stirred at room temperature for 2 hours. TLC indicated complete consumption of starting material. The reaction mixture was concentrated under reduced pressure to afford (4-(1,6-dimethyl-7-oxo-6,7-dihydro-1H-pyrazolo[3,4-c]pyridin-4-yl)-2,6-dimethoxybenzyl)glycine (32, 300 mg, crude) as a brown liquid. LCMS (ES+): m/z 387 [M+H]+

### Targeting Ligand Synthesis 4

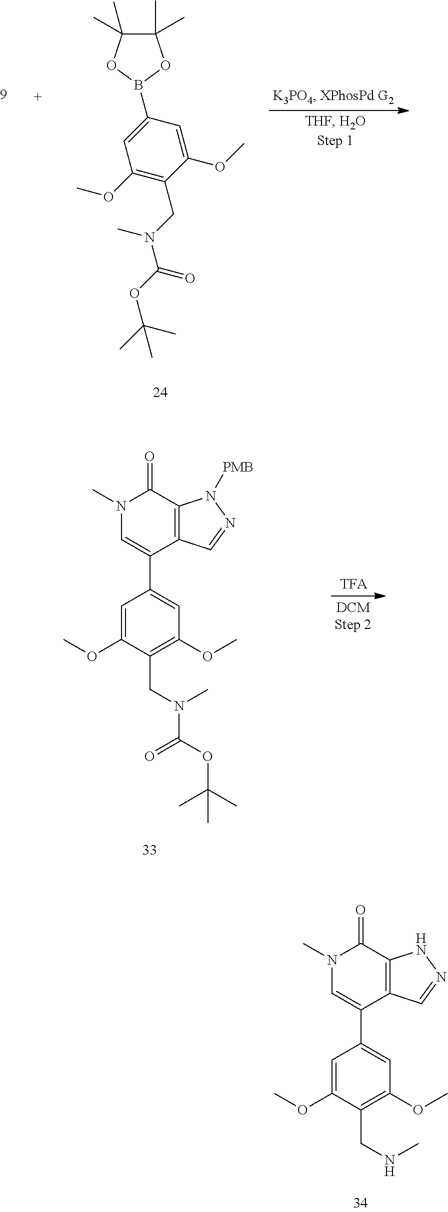

Step 1: Into a 20 mL sealed tube containing a mixture of tert-butyl N-[[2,6-dimethoxy-4-(4,4,5,5-tetramethyl-1,3,2-dioxaborolan-2-yl)phenyl]methyl]-N-methyl-carbamate (24, 58.49 mg, 143.60 μmol) and 4-bromo-1-[(4-methoxyphenyl)methyl]-6-methyl-pyrazolo[3,4-c]pyridin-7-one (9, 50 mg, 143.60 μmol) in THF (5 mL) and water (1 mL) was added potassium phosphate tribasic (60.96 mg, 287.20 μmol). Argon gas was bubbled through the reaction mixture for 5 minutes, followed by the addition of XPhos-Pd-G2 (3.39 mg, 4.31 μmol) and the resulting suspension was purged with argon gas for an additional 5 minutes. The contents were heated at 80° C. for 4 hours. After completion, the reaction was filtered over a pad of Celite® and the filtrate was extracted with EtOAc twice. The combined organic layers were removed under reduced pressure to afford crude tert-butyl N-[[2,6-dimethoxy-4-[1-[(4-methoxyphenyl)methyl]-6-methyl-7-oxo-pyrazolo[3,4-c]pyridin-4-yl]phenyl]methyl]-N-methyl-carbamate (33, 70 mg, crude) that was used in the next step without further purification. LCMS (ES+): m/z 549 [M+H]+

Step 2: tert-Butyl N-[[2,6-dimethoxy-4-[1-[(4-methoxyphenyl)methyl]-6-methyl-7-oxo-pyrazolo[3,4-c]pyridin-4-yl]phenyl]methyl]-N-methyl-carbamate (33, 50 mg, 91.14 μmol) was taken up in a 10 ml sealed tube. TFA (4.44 g, 38.94 mmol, 3 mL) was added and the reaction was stirred at 80° C. for 2 hours. After completion of the reaction, TFA was evaporated under reduced pressure to afford a crude product that was purified by reverse phase prep purification (SUNFIRE OBD C18(100×30)MM 5μ) Mobile phase: A: 0.1% TFA in water B: ACN) to obtain 4-[3,5-dimethoxy-4-(methylaminomethyl)phenyl]-6-methyl-1H-pyrazolo[3,4-c]pyridin-7-one (34, 7 mg, 21.32 μmol, 23.39% yield) as an off-white colored solid. LCMS (ES+): m/z 329 [M+H]⁺

### Targeting Ligand Synthesis 5

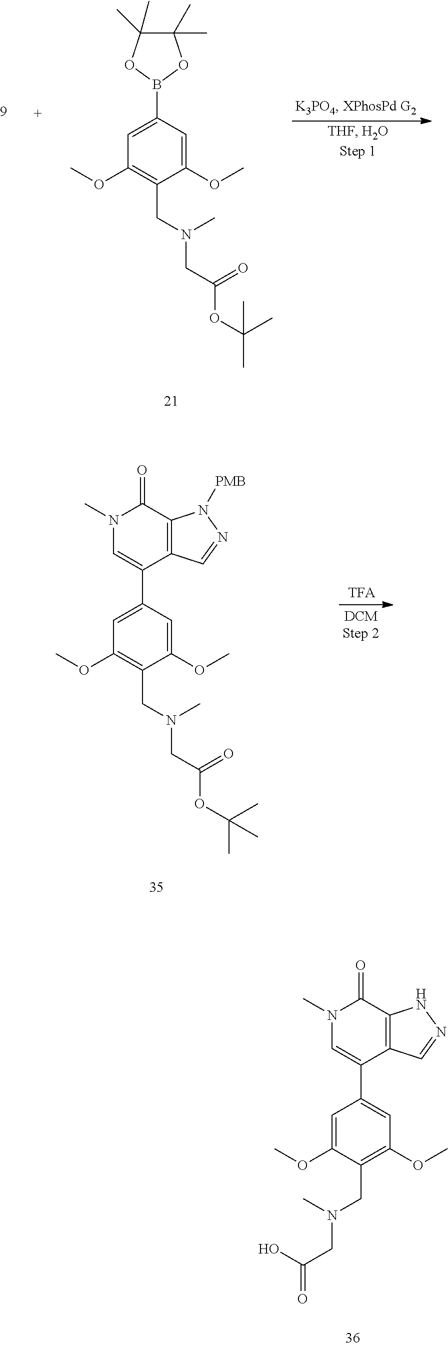

Step 1: Into a 20 mL sealed tube containing a mixture of tert-butyl N-(2,6-dimethoxy-4-(4,4,5,5-tetramethyl-1,3,2-dioxaborolan-2-yl)benzyl)-N-methylglycinate (21, 181.51 mg, 430.79 μmol) and 4-bromo-1-[(4-methoxyphenyl)methyl]-6-methyl-pyrazolo[3,4-c]pyridin-7-one (9, 0.15 g, 430.79 μmol) in THF (5 mL) and water (1 mL) was added potassium phosphate tribasic (182.89 mg, 861.59 μmol). Argon gas was bubbled through the reaction mixture for 5 minutes followed by the addition of XPhos-Pd-G2 (10.17 mg, 12.92 μmol) and again the resulting suspension was purged with argon gas for an additional 5 minutes. The reaction was heated at 80° C. for 2 hours. After completion, the reaction was filtered through a pad of Celite® and the obtained filtrate was extracted with EtOAc. The combined organic layers were removed under reduced pressure to afford tert-butyl 2-[[2,6-dimethoxy-4-[1-[(4-methoxyphenyl)methyl]-6-methyl-7-oxo-pyrazolo[3,4-c]pyridin-4-yl]phenyl]methyl-methyl-amino]acetate (35, 0.3 g, crude) that was used in the next step without further purification. LCMS (ES+): m/z 563 [M+H]+

Step 2: tert-Butyl 2-[[2,6-dimethoxy-4-[1-[(4-methoxyphenyl)methyl]-6-methyl-7-oxo-pyrazolo[3,4-c]pyridin-4-yl]phenyl]methyl-methyl-amino]acetate (35, 0.3 g, 533.19 μmol) was dissolved in DCM (5 mL) and TFA (2.96 g, 25.96 mmol, 2 mL) was added at 0° C. The reaction mixture was allowed to stir at room temperature for 1 hour. After completion, the volatiles were evaporated under reduced pressure. Excess solvent was evaporated under high vacuum to obtain crude N-(2,6-dimethoxy-4-(6-methyl-7-oxo-6,7-dihydro-1H-pyrazolo[3,4-c]pyridin-4-yl)benzyl)-N-methylglycine (36, 50 mg, crude) that was used in the next step without any further purification. LCMS (ES+): m/z 387 [M+H]⁺

### Targeting Ligand Synthesis 6

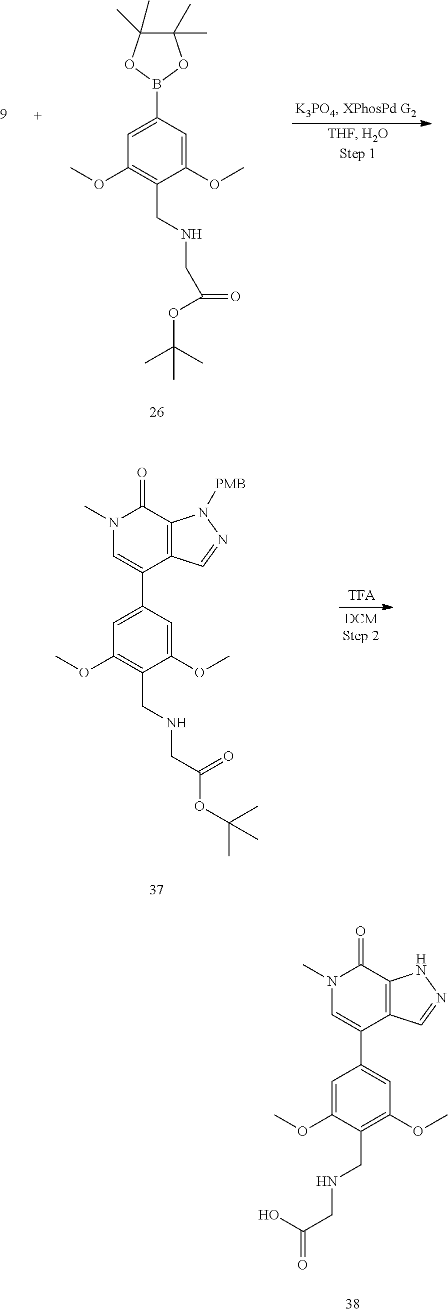

Step 1: Into a 20 mL sealed tube containing a mixture of tert-butyl (2,6-dimethoxy-4-(4,4,5,5-tetramethyl-1,3,2-dioxaborolan-2-yl)benzyl)glycinate (26, 350.93 mg, 861.59 μmol) and 4-bromo-1-[(4-methoxyphenyl)methyl]-6-methyl-pyrazolo[3,4-c]pyridin-7-one (9, 0.3 g, 861.59 μmol) in THF (10 mL) and water (2 mL) was added potassium phosphate tribasic (365.78 mg, 1.72 mmol). Argon gas was bubbled through the reaction mixture for 5 minutes followed by the addition of XPhos-Pd-G2 (40.67 mg, 25.85 μmol). The resulting suspension was purged with argon gas for an additional 5 minutes. The reaction was heated at 80° C. for 4 hours. After completion, the reaction was filtered through a pad of Celite® and the filtrate was extracted with EtOAc twice. The combined organic layers were evaporated under reduced pressure to afford tert-butyl (2,6-dimethoxy-4-(1-(4-methoxybenzyl)-6-methyl-7-oxo-6,7-dihydro-1H-pyrazolo[3,4-c]pyridin-4-yl)benzyl)glycinate (37, 0.5 g, crude) that was used in the next step without further purification. LCMS (ES+): m/z 549 [M+H]+

Step 2: tert-Butyl 2-[[2,6-dimethoxy-4-[1-[(4-methoxyphenyl)methyl]-6-methyl-7-oxo-pyrazolo[3,4-c]pyridin-4-yl]phenyl]methylamino]acetate (37, 0.5 g, 911.36 μmol) was dissolved in TFA (6.16 g, 54.00 mmol, 4.16 mL) and the reaction was stirred at 80° C. for 2 hours. After completion, the volatiles were evaporated under reduced pressure. The material was purified by reverse phase prep purification (SUNFIRE OBD C18(100×30)MM 5μ) Mobile phase: A: 0.1% TFA in water B: ACN) to obtain (2,6-dimethoxy-4-(1-(4-methoxybenzyl)-6-methyl-7-oxo-6,7-dihydro-1H-pyrazolo[3,4-c]pyridin-4-yl)benzyl) (38, 0.15 g, 304.55 μmol, 33.42% yield). LCMS (ES+): m/z 373 [M+H]⁺

### Targeting Ligand Synthesis 7

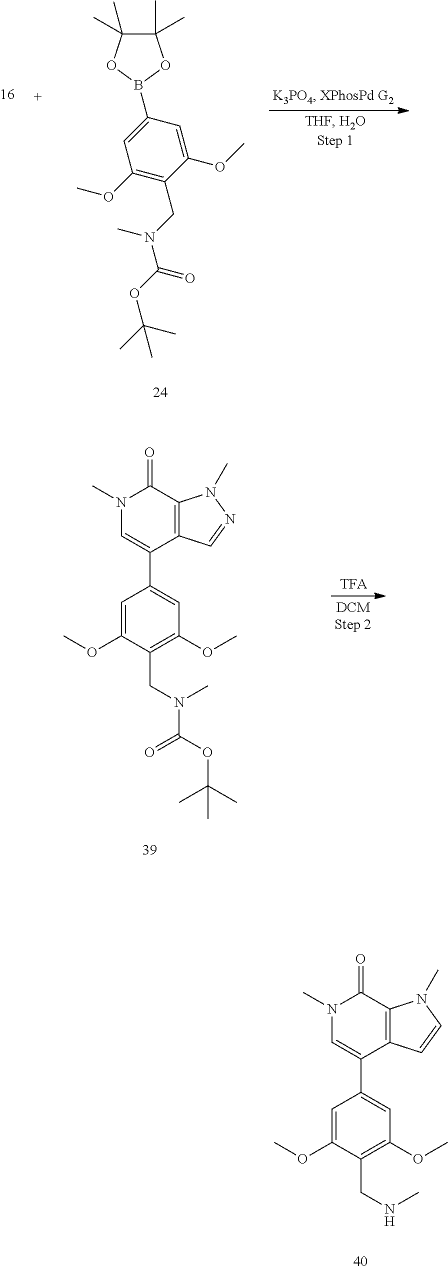

Step 1 and 2: Into a 20 mL sealed tube containing a mixture of 4-bromo-1,6-dimethyl-pyrrolo[2,3-c]pyridin-7-one (16, 59.37 mg, 246.26 μmol) and tert-butyl N-[[2,6-dimethoxy-4-(4,4,5,5-tetramethyl-1,3,2-dioxaborolan-2-yl)phenyl]methyl]-N-methyl-carbamate (24, 120.36 mg, 295.51 μmol) in water (1 mL) and THF (4 mL) was added potassium phosphate tribasic (130.68 mg, 615.65 μmol). Argon gas was bubbled through reaction mixture for 10 minutes, followed by addition of XPhos-Pd-G2 (38.75 mg, 24.63 μmol). The resulting suspension was purged with argon gas for an additional 10 minutes and the contents were stirred at 80° C. under the closed condition for 2 hours. TLC indicated complete consumption of starting material. The reaction mixture was passed through a pad of Celite® and the filtrate was concentrated under reduced pressure to afford crude material that was dissolved in DCM (5 mL). TFA (140.40 mg, 1.23 mmol, 94.86 uL) was added and the reaction was stirred for 30 minutes. The resulting mixture was concentrated under reduced pressure and purified by prep-HPLC SUNFIRE OBD C18 (100×30)MM 5μ) Mobile phase: A: 0.1% TFA in water B: ACN to afford 4-[3,5-dimethoxy-4-(methylaminomethyl)phenyl]-1,6-dimethyl-pyrrolo[2,3-c]pyridin-7-one (40, 11 mg, 30.67 μmop as a white solid. LCMS (ES+): m/z 342 [M+H]⁺

### Targeting Ligand Synthesis 8

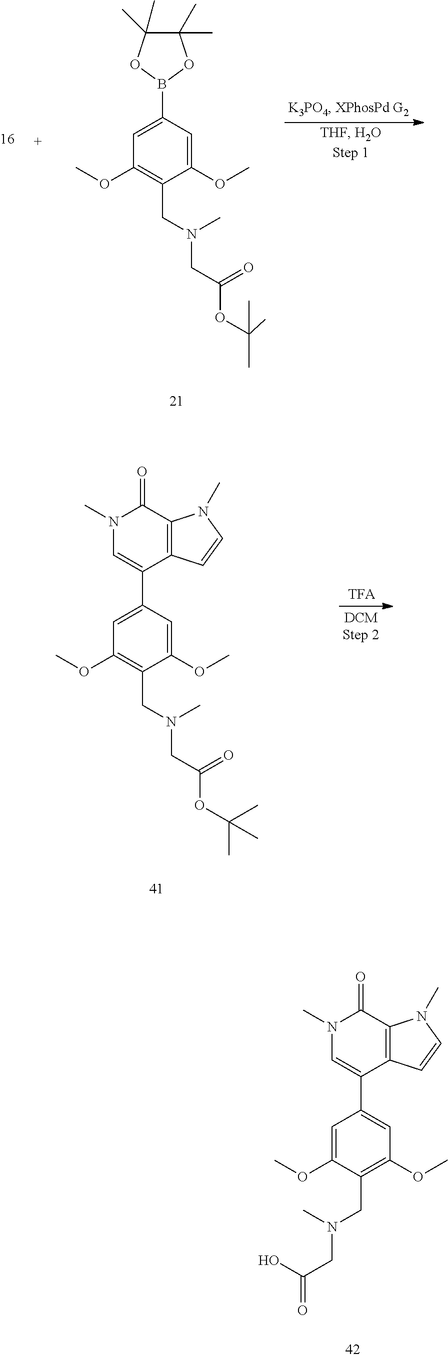

Step 1: Into a 50 mL sealed tube containing a mixture of tert-butyl N-(2,6-dimethoxy-4-(4,4,5,5-tetramethyl-1,3,2-dioxaborolan-2-yl)benzyl)-N-methylglycinate and compound 16 (4-bromo-1,6-dimethyl-pyrrolo[2,3-c]pyridin-7-one) (84.19 mg, 349.23 μmol) in THF (4 mL) and water (0.5 mL) was added potassium phosphate tribasic (148.26 mg, 698.47 μmol). Argon gas was bubbled through the reaction mixture for 10 minutes, followed by the addition of XPhos-Pd-G2 (54.96 mg, 34.92 μmol). The resulting suspension was purged with argon gas for an additional 10 minutes. The contents were stirred at 80° C. for 2 hours. The reaction mixture was passed through a pad of Celite® and the filtrate was concentrated under reduced pressure to afford a crude solid. The crude material was purified by silica-gel (230-400 mesh) chromatography with 1:9 MeOH/DCM to provide tert-butyl 2-[[4-(1,6-dimethyl-7-oxo-pyrrolo[2,3-c]pyridin-4-yl)-2,6-dimethoxy-phenyl]methyl-methyl-amino]acetate (41, 50 mg, 109.76 μmol, 31.43% yield) as a brown liquid. LCMS (ES+): m/z 456 [M+H]+

Step 2: To a stirred solution of tert-butyl 2-[[4-(1,6-dimethyl-7-oxo-pyrrolo[2,3-c]pyridin-4-yl)-2,6-dimethoxy-phenyl]methyl-methyl-amino]acetate (41, 200 mg, 439.03 μmol) in dichloromethane (20 mL) was added trifluoroacetic acid (50.06 mg, 439.03 μmol, 33.82 uL) at 0° C. and the reaction was stirred at ambient temperature for 4 hours. The progress of the reaction was monitored by TLC. The reaction mixture was concentrated under reduced pressure to afford 24-[4-(1,6-dimethyl-7-oxo-pyrrolo[2,3-c]pyridin-4-yl)-2,6-dimethoxy-phenyl]methyl-methyl-amino]acetic acid (42, 170 mg, 425.60 μmol, 96.94% yield) that was carried forward without further purification. LCMS (ES+): m/z 400 [M+H]+

### Targeting Ligand Synthesis 9

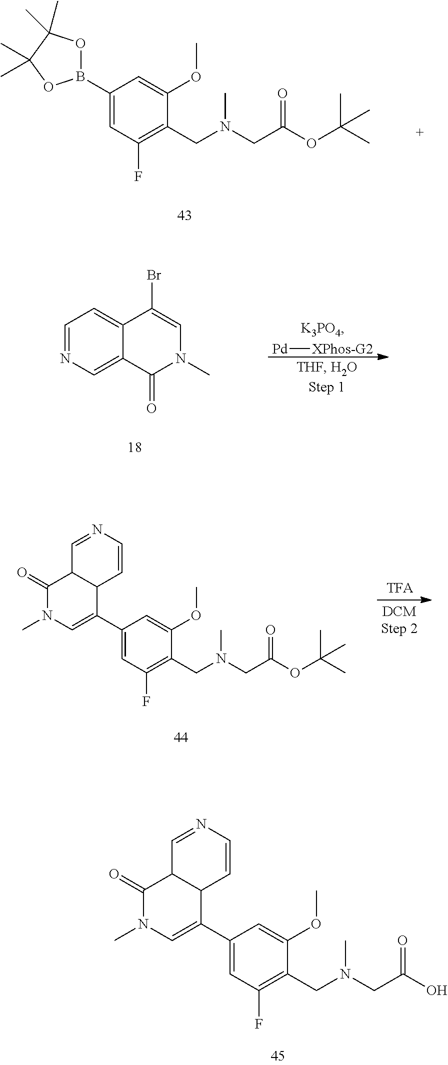

Step 1: A solution of tert-butyl 2-[[2-fluoro-6-methoxy-4-(4,4,5,5-tetramethyl-1,3,2-dioxaborolan-2-yl)phenyl]methyl-methyl-amino]acetate (43, 200 mg, 488.64 μmol), 4-bromo-2-methyl-2,7-naphthyridin-1-one (18, 128.50 mg, 537.50 μmol), potassium phosphate tribasic anhydrous (207.44 mg, 977.28 μmol) in THF (5 mL) and water (1 mL) in a sealed tube was purged with argon for 5 minutes. XPhos-Pd-G2 (11.53 mg, 14.66 μmol) was added and the reaction was stirred for 2 hours at 70° C. The reaction mixture was cooled to ambient temperature, diluted with water and extracted with ethyl acetate (3×25 mL). The combined organic extracts were washed with brine solution, dried over anhydrous sodium sulfate, filtered, and the excess solvent was evaporated under reduced pressure. The resulting crude material was purified by column chromatography on silica eluted with 0-5% methanol in dichloromethane to yield tert-butyl 2-[[2-fluoro-6-methoxy-4-(2-methyl-1-oxo-4a,8a-dihydro-2,7-naphthyridin-4-yl)phenyl]methyl-methyl-amino]acetate (44, 230 mg, 414.87 μmol, 84.90% yield) as a pale yellow oil. LCMS (ES+): m/z 444 [M+H]+

Step 2: To a stirred solution of tert-butyl 2-[[2-fluoro-6-methoxy-4-(2-methyl-1-oxo-4a,8a-dihydro-2,7-naphthyridin-4-yl)phenyl]methyl-methyl-amino]acetate (44, 100 mg, 225.47 μmol) in DCM (5 mL) was added trifluoroacetic acid (1.48 g, 12.98 mmol, 1 mL) and the reaction mixture was stirred for 1 hour at 25° C. The reaction mixture was concentrated under reduced pressure to yield 2-[[2-fluoro-6-methoxy-4-(2-methyl-1-oxo-4a,8a-dihydro-2,7-naphthyridin-4-yl)phenyl]methyl-methyl-amino]acetic acid (45, 100 mg, 185.85 μmol, 82.43% yield). LCMS (ES+): m/z 388 [M+H]+

### Targeting Ligand Synthesis 10

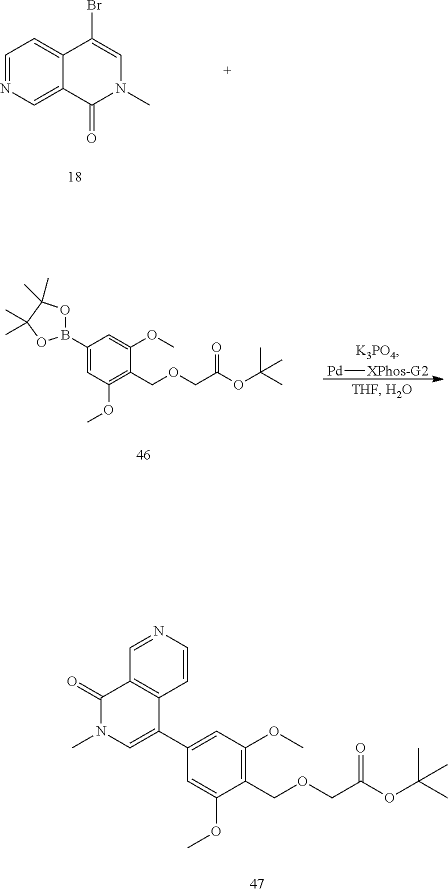

Into a 50 mL sealed tube containing a mixture of 4-bromo-2-methyl-2,7-naphthyridin-1-one (18, 79.67 mg, 333.27 μmol) and tert-butyl 2-[[2,6-dimethoxy-4-(4,4,5,5-tetramethyl-1,3,2-dioxaborolan-2-yl)phenyl]methoxy]acetate (46, 136.07 mg, 333.27 μmol) in THF (4 mL) and water (0.5 mL) was added potassium phosphate tribasic anhydrous (141.49 mg, 666.54 μmol). Argon gas was bubbled through the reaction mixture for 10 minutes and XPhos-Pd-G2 (52.44 mg, 33.33 μmol) was added. Again the resulting suspension was purged with argon gas for an additional 10 minutes. The contents were stirred at 80° C. under closed conditions. TLC indicated complete consumption of stating materials after 2 hours. The reaction mixture was passed through a pad of Celite and the filtrate was concentrated under reduced pressure to afford the crude material. The crude material was purified by a silica-gel (230-400 mesh) with 100% EtOAc ether to generate tert-butyl 2-[[2,6-dimethoxy-4-(2-methyl-1-oxo-2,7-naphthyridin-4-yl)phenyl]methoxy]acetate (47, 100 mg, 227.02 μmol, 68.12% yield) as a white solid. LCMS (ES+): m/z 441 [M+H]+

### Targeting Ligand Synthesis 11

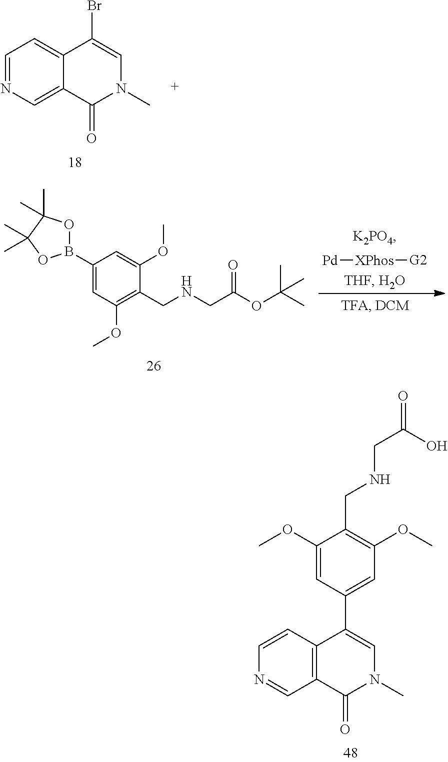

Into a 20 mL sealed tube containing a mixture of 4-bromo-2-methyl-2,7-naphthyridin-1-one (18, 106.23 mg, 444.36 mol) and tert-butyl 2-[[2,6-dimethoxy-4-(4,4,5,5-tetramethyl-1,3,2-dioxaborolan-2-yl)phenyl]methylamino]acetate (26, 199.09 mg, 488.80 mol) in THF (4 mL) and water (0.5 mL) was added potassium phosphate tribasic anhydrous (188.65 mg, 888.72 mol). Argon gas was bubbled through the reaction mixture for 10 minutes and XPhos-Pd-G2 (69.92 mg, 44.44 mol) was added. The resulting suspension was purged with argon gas for an additional 10 minutes and the contents were stirred at 80° C. under closed conditions. TLC indicated complete consumption of stating materials after 2 hours. The reaction mixture was passed through a pad of Celite, and the filtrate was concentrated under reduced pressure to afford the crude mass. The crude material was dissolved in DCM (4 mL) and TFA (50.67 mg, 444.36 mol, 34.23 uL) was added. The reaction stirred at room temperature for 2 hours. The reaction mixture was concentrated and purified by prep-HPLC to afford 2-[[2,6-dimethoxy-4-(2-methyl-1-oxo-2,7-naphthyridin-4-yl)phenyl]methylamino]acetic acid (48, 30 mg, 78.25 mol, 17.61% yield). LCMS (ES+): m/z 384 [M+H]+

### Targeting Ligand Synthesis 12

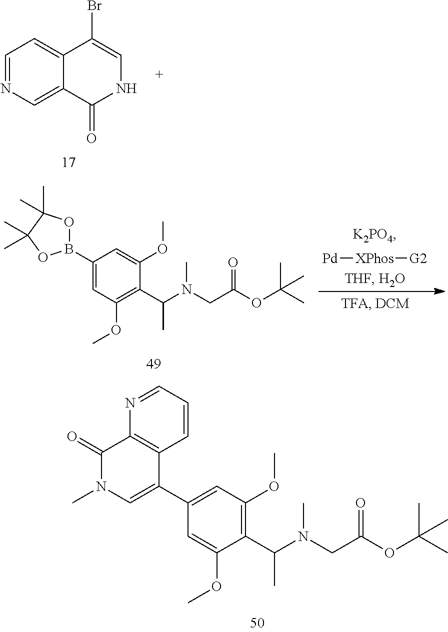

Into a 20 mL sealed tube containing a mixture of 4-bromo-2H-2,7-naphthyridin-1-one (17, 25 mg, 111.09 μmol) and tert-butyl 2-[1-[2,6-dimethoxy-4-(4,4,5,5-tetramethyl-1,3,2-dioxaborolan-2-yl)phenyl]ethyl-methyl-amino]acetate (49, 48.36 mg, 111.09 μmol) in water (0.5 mL) and THF (4 mL) was added potassium phosphate tribasic anhydrous (47.16 mg, 222.18 μmol). Argon gas was bubbled through the reaction mixture for 10 minutes and XPhos-Pd-G2 (17.48 mg, 11.11 μmol) was added. The resulting suspension was purged with argon gas for an additional 10 minutes. The contents were stirred at 80° C. under closed conditions. The reaction mixture was passed through a pad of Celite and the filtrate was concentrated under reduced pressure to afford tert-butyl 2-[1-[2,6-dimethoxy-4-(2-methyl-1-oxo-2,7-naphthyridin-4-yl)phenyl]ethyl-methyl-amino]acetate (50, 50 mg, crude). LCMS (ES+): m/z 468 [M+H]+

### BRD9 Degrader Synthesis

### Degrader Synthesis 1: Compound 200

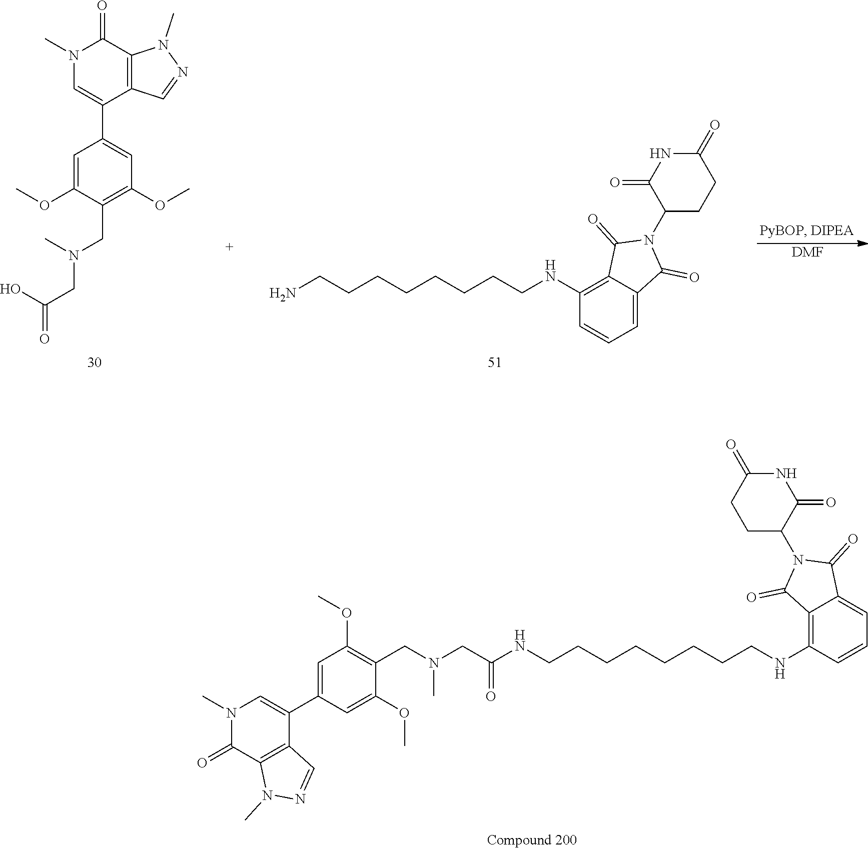

Into a 25 mL single-necked round-bottomed flask containing a well-stirred solution of 2-[[4-(1,6-dimethyl-7-oxo-pyrazolo[3,4-c]pyridin-4-yl)-2,6-dimethoxy-phenyl]methyl-methyl-amino]acetic acid (30, 50 mg, 124.87 μmol) and 4-(8-aminooctylamino)-2-(2,6-dioxo-3-piperidyl)isoindoline-1,3-dione (51, 50.01 mg, 124.87 μmol) in DMF (5 mL) were added DIPEA (48.41 mg, 374.60 μmol, 65.25 uL) and PyBOP (77.98 mg, 149.84 μmol) under nitrogen atmosphere at room temperature. The resulting mixture was stirred at room temperature for 16 hours. After completion of the reaction, 20 mL of cold water was added and the reaction was stirred for 10 minutes. The resulting solid was filtered and dried to afford crude product that was purified by reverse phase prep purification (SUNFIRE OBD C18(100×30)MM 5μ) Mobile phase: A: 0.1% TFA in water B: ACN) to obtain 2-[[4-(1,6-dimethyl-7-oxo-pyrazolo[3,4-c]pyridin-4-yl)-2,6-dimethoxy-phenyl]methyl-methyl-amino]-N-[8-[[2-(2,6-dioxo-3-piperidyl)-1,3-dioxo-isoindolin-4-yl]amino]octyl]acetamide (Compound 200, 28 mg, 35.77 μmol, 28.64% yield). ¹H NMR (400 MHz, DMSO-d₆) δ 11.10 (s, 1H), 9.47 (s, 1H), 8.38 (t, J=5.5 Hz, 1H), 8.11 (d, J=1.5 Hz, 1H), 7.64 (d, J=1.6 Hz, 1H), 7.57 (t, J=8.1 Hz, 1H), 7.07 (d, J=8.5 Hz, 1H), 7.02 (dd, J=7.1, 1.6 Hz, 1H), 6.93 (s, 2H), 6.51 (t, J=5.9 Hz, 1H), 5.05 (dd, J=12.8, 5.3 Hz, 1H), 4.32 (s, 4H), 3.91 (s, 5H), 3.80 (d, J=4.5 Hz, 2H), 3.60 (s, 3H), 3.27 (q, J=6.7 Hz, 3H), 3.12 (p, J=6.9 Hz, 2H), 3.04-2.98 (m, 1H), 2.88 (ddd, J=17.7, 13.8, 5.3 Hz, 1H), 2.72 (d, J=4.1 Hz, 3H), 2.63-2.56 (m, 2H), 2.07-1.98 (m, 1H), 1.55 (t, J=7.1 Hz, 2H), 1.41 (s, 2H), 1.27 (d, J=11.0 Hz, 7H). LCMS (ES+): m/z 783 [M+H]⁺

### Degrader Synthesis 2: Compound 201

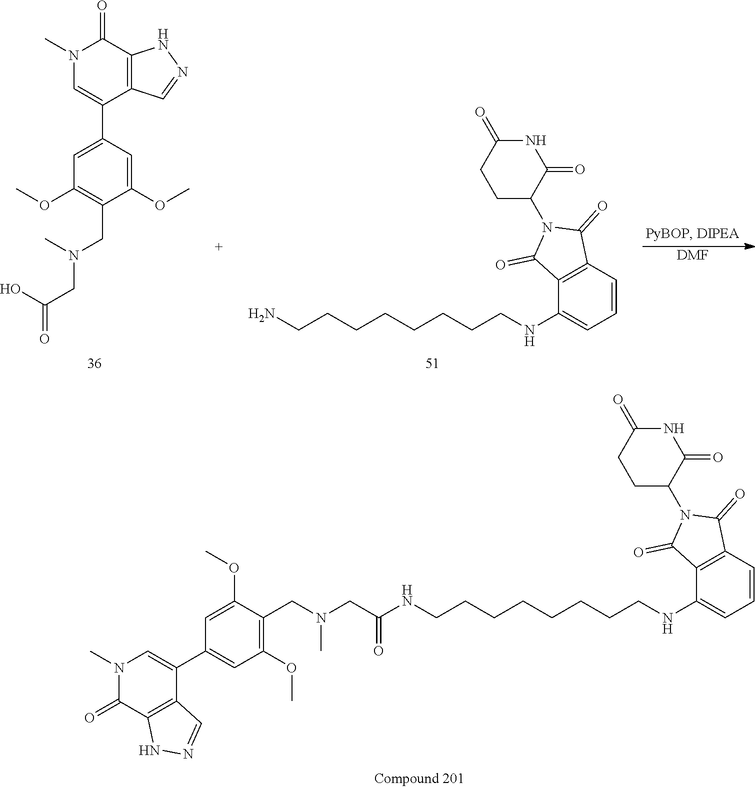

Into a 25 mL single-necked round-bottomed flask containing a well-stirred solution of 2-[[2,6-dimethoxy-4-(6-methyl-7-oxo-1H-pyrazolo[3,4-c]pyridin-4-yl)phenyl]methyl-methyl-amino]acetic acid (36, 48.24 mg, 124.85 μmol) and 4-(8-aminooctylamino)-2-(2,6-dioxo-3-piperidyl)isoindoline-1,3-dione (51, 50 mg, 124.85 μmol) in DMF (5 mL) were added DIPEA (48.41 mg, 374.56 μmol, 65.24 uL) and PyBOP (77.97 mg, 149.82 μmol) under nitrogen atmosphere at room temperature. The resulting mixture was stirred at room temperature for 16 hours. After completion of the reaction, 20 mL of cold water was added and the reaction was stirred for 10 minutes. The resulting solid was filtered and dried to afford crude product that was purified by reverse phase prep purification (SUNFIRE OBD C18(100×30)MM 5μ) Mobile phase: A: 0.1% TFA in water B: ACN) to obtain 2-[[2,6-dimethoxy-4-(6-methyl-7-oxo-1H-pyrazolo[3,4-c]pyridin-4-yl)phenyl]methyl-methyl-amino]-N-[8-[[2-(2,6-dioxo-3-piperidyl)-1,3-dioxo-isoindolin-4-yl]amino]octyl]acetamide (Compound 201, 8 mg, 10.41 μmol, 8.33% yield). ¹H NMR (400 MHz, DMSO-d₆) δ 11.10 (s, 1H), 9.47 (s, 1H), 8.44-8.37 (m, 1H), 8.22 (s, 1H), 7.64 (s, 1H), 7.57 (dd, J=8.6, 7.1 Hz, 1H), 7.07 (d, J=8.6 Hz, 1H), 7.02 (d, J=7.0 Hz, 1H), 6.95 (s, 2H), 6.51 (t, J=5.9 Hz, 1H), 5.04 (dd, J=12.7, 5.4 Hz, 1H), 4.32 (s, 2H), 3.92 (s, 6H), 3.80 (s, 2H), 3.62 (s, 3H), 3.27 (q, J=6.7 Hz, 2H), 3.12 (p, J=7.4, 6.7 Hz, 2H), 2.93-2.82 (m, 2H), 2.72 (d, J=3.7 Hz, 3H), 2.62-2.54 (m, 2H), 2.11-1.96 (m, 1H), 1.55 (p, J=6.6 Hz, 2H), 1.45-1.37 (m, 2H), 1.34-1.21 (m, 8H). LCMS (ES+): m/z 769 [M+H]⁺

### Degrader Synthesis 3: Compound 202

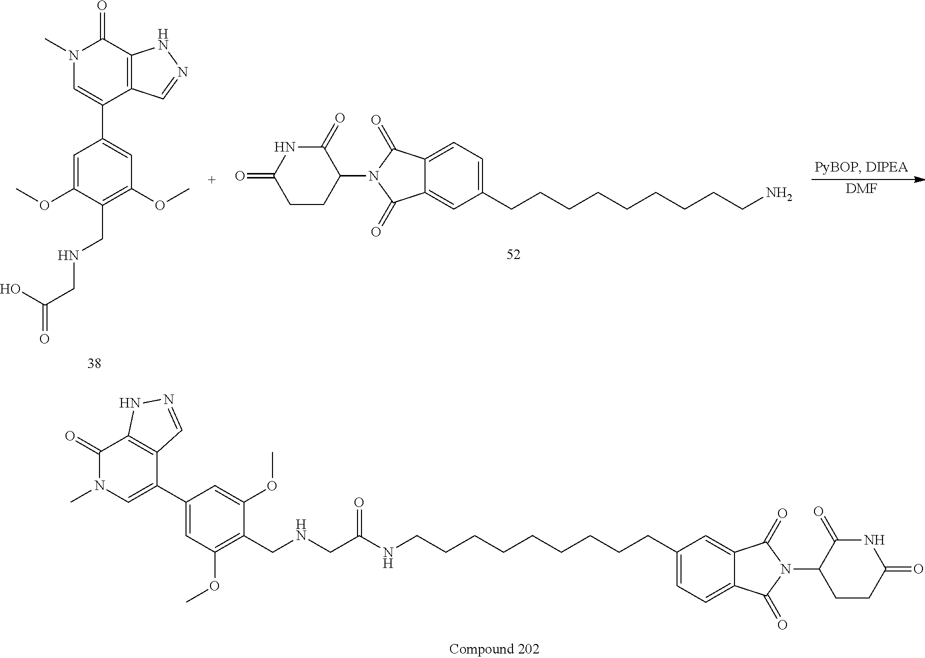

Into a 25 mL single-necked round-bottomed flask containing a well-stirred solution of 2-[[2,6-dimethoxy-4-(6-methyl-7-oxo-1H-pyrazolo[3,4-c]pyridin-4-yl)phenyl]methylamino]acetic acid (38, 30 mg, 80.56 μmol) and 5-(9-aminononyl)-2-(2,6-dioxo-3-piperidyl)isoindoline-1,3-dione (52, 32.18 mg, 80.56 μmol) in DMF was added DIPEA (10.41 mg, 80.56 μmol, 14.03 uL) and PyBOP (41.92 mg, 80.56 μmol) under nitrogen atmosphere at room temperature, the resulting mixture was stirred at room temperature for 16 hours. After completion of the reaction, 20 mL of cold water was added and the reaction stirred for 10 minutes. The resulting solid was filtered and dried to afford crude product that was purified by reverse phase prep purification (SUNFIRE OBD C18(100×30)MM 5μ) Mobile phase: A: 0.1% TFA in water B: ACN) to obtain 2-[[2,6-dimethoxy-4-(6-methyl-7-oxo-1H-pyrazolo[3,4-c]pyridin-4-yl)phenyl]methylamino]-N-[9-[2-(2,6-dioxo-3-piperidyl)-1,3-dioxo-isoindolin-5-yl]nonyl]acetamide (Compound 202, 6 mg, 7.96 μmol, 9.88% yield). ¹H NMR (400 MHz, DMSO-d₆) δ 11.11 (s, 1H), 8.73 (s, 2H), 8.28 (t, J=5.6 Hz, 1H), 8.18 (s, 1H), 7.83 (d, J=7.6 Hz, 1H), 7.75 (s, 1H), 7.69 (d, J=7.6 Hz, 1H), 7.63 (s, 1H), 7.21 (s, 0.5H), 7.08 (s, 0.5H), 6.95 (s, 0.5H), 6.93 (s, 2H), 5.13 (dd, J=12.8, 5.3 Hz, 1H), 4.14 (s, 2H), 3.92 (s, 6H), 3.62 (s, 3H), 3.54 (d, J=5.5 Hz, 2H), 3.12 (q, J=6.6 Hz, 2H), 2.95-2.82 (m, 1H), 2.77 (t, J=7.5 Hz, 2H), 2.64-2.56 (m, 1H), 2.10-1.99 (m, 1H), 1.65-1.54 (m, 2H), 1.46-1.34 (m, 2H), 1.25 (d, J=10.4 Hz, 11H). LCMS (ES+): m/z 754 [M+H]⁺

### Degrader Synthesis 4: Compound 203

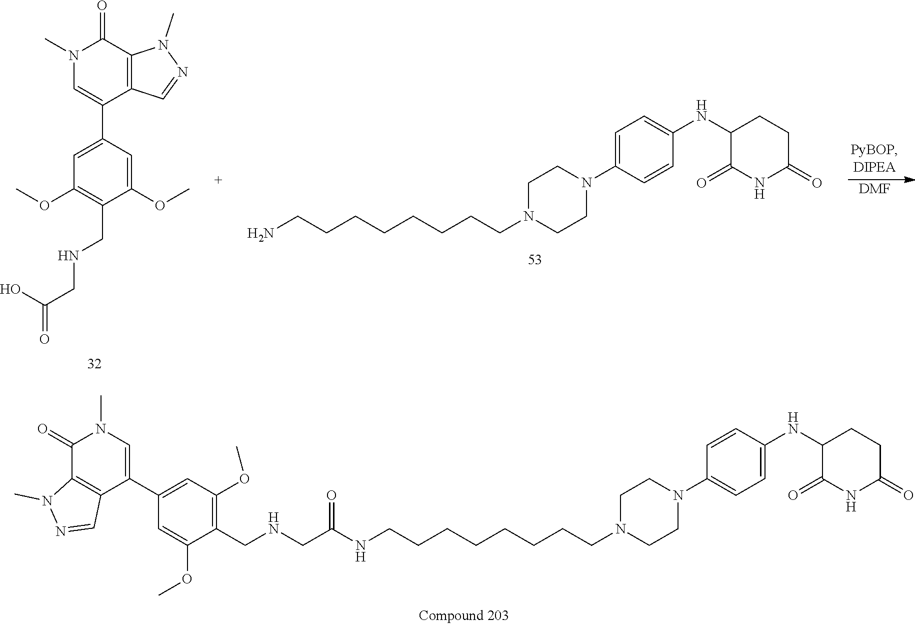

Into a 25 mL single-necked round-bottomed flask containing a well-stirred solution of 2-[[4-(1,6-dimethyl-7-oxo-pyrazolo[3,4-c]pyridin-4-yl)-2,6-dimethoxy-phenyl]methylamino]acetic acid (32, 23.25 mg, 60.16 μmol) and 3-[4-[4-(8-aminooctyl)piperazin-1-yl]anilino]piperidine-2,6-dione (53, 25 mg, 60.16 μmol) in DMF (2 mL) were added DIPEA (23.33 mg, 180.47 μmol, 31.44 uL) and PyBoP (46.96 mg, 90.24 μmol) under nitrogen atmosphere at room temperature. The resulting mixture was stirred at room temperature for 16 hours. The reaction mixture was concentrated and purified by prep-HPLC SUNFIRE OBD C18(100×30)MM 5μ) Mobile phase: A: 0.1% TFA in water B: ACN to afford 4 mg of 2-[[4-(1,6-dimethyl-7-oxo-pyrazolo[3,4-c]pyridin-4-yl)-2,6-dimethoxy-phenyl]methylamino]-N-[8-[4-[4-[(2,6-dioxo-3-piperidyl)amino]phenyl]piperazin-1-yl]octyl]acetamide (Compound 203, 4 mg, 4.94 μmol, 8.22% yield) as a brown liquid. LCMS (ES+): m/z 784 [M+H]⁺

### Degrader Synthesis 5: Compound 204

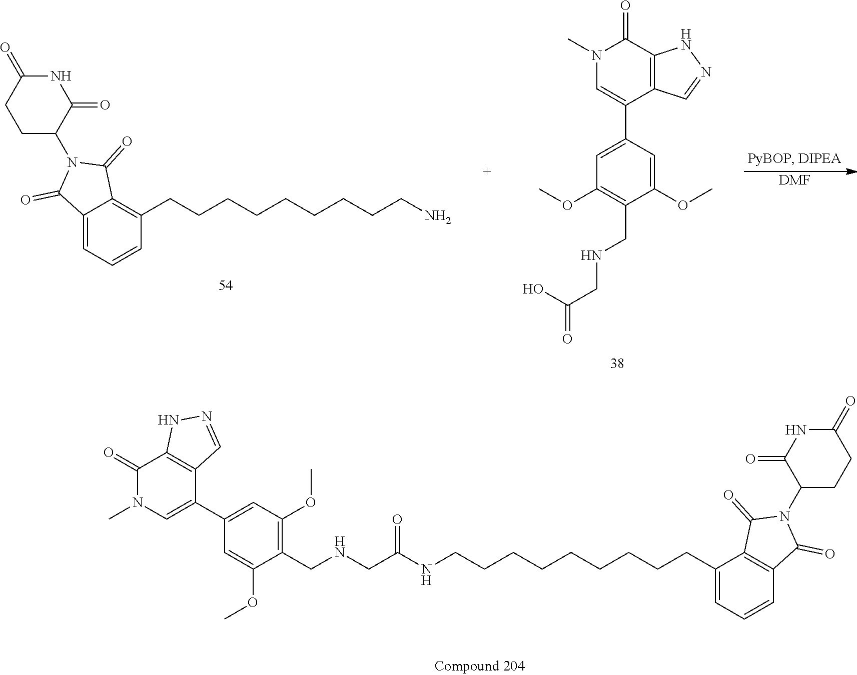

Into a 25 mL single-necked round-bottomed flask containing a well-stirred solution of 4-(9-aminononyl)-2-(2,6-dioxo-3-piperidyl)isoindoline-1,3-dione (54, 25 mg, 62.58 μmol) and 2-[[2,6-dimethoxy-4-(6-methyl-7-oxo-1H-pyrazolo[3,4-c]pyridin-4-yl)phenyl]methylamino]acetic acid (38, 23.30 mg, 62.58 μmol) in DMF (2 mL) were added DIPEA (24.26 mg, 187.74 μmol, 32.70 uL) and PyBoP (48.85 mg, 93.87 μmol) under nitrogen atmosphere at room temperature. The resulting mixture was stirred at room temperature for 16 hours. The reaction mixture was concentrated and purified by prep-HPLC SUNFIRE OBD C18(100×30)MM 5μ) Mobile phase: A: 0.1% TFA in water B: ACN to afford 8 mg of 2-[[2,6-dimethoxy-4-(6-methyl-7-oxo-1H-pyrazolo[3,4-c]pyridin-4-yl)phenyl]methylamino]-N-[9-[2-(2,6-dioxo-3-piperidyl)-1,3-dioxo-isoindolin-4-yl]nonyl]acetamide (Compound 204, 8 mg, 10.44 μmol, 16.68% yield) as a colorless gummy liquid. ¹H NMR (400 MHz, Methanol-d₄) δ 8.12 (s, 1H), 7.70-7.67 (m, 2H), 7.60 (q, J=4.1 Hz, 1H), 7.46 (s, 1H), 6.95 (s, 2H), 5.11 (dd, J=12.5, 5.4 Hz, 1H), 4.35 (s, 2H), 3.98 (d, J=1.4 Hz, 6H), 3.72-3.67 (m, 5H), 3.21 (t, J=7.3 Hz, 2H), 3.06 (t, J=7.8 Hz, 2H), 2.92-2.81 (m, 1H), 2.79-2.67 (m, 2H), 2.17-2.08 (m, 1H), 1.63 (p, J=7.2 Hz, 2H), 1.53-1.43 (m, 2H), 1.30 (d, J=16.1 Hz, 10H). LCMS (ES+): m/z 754 [M+H]⁺

### Degrader Synthesis 6: Compound 205

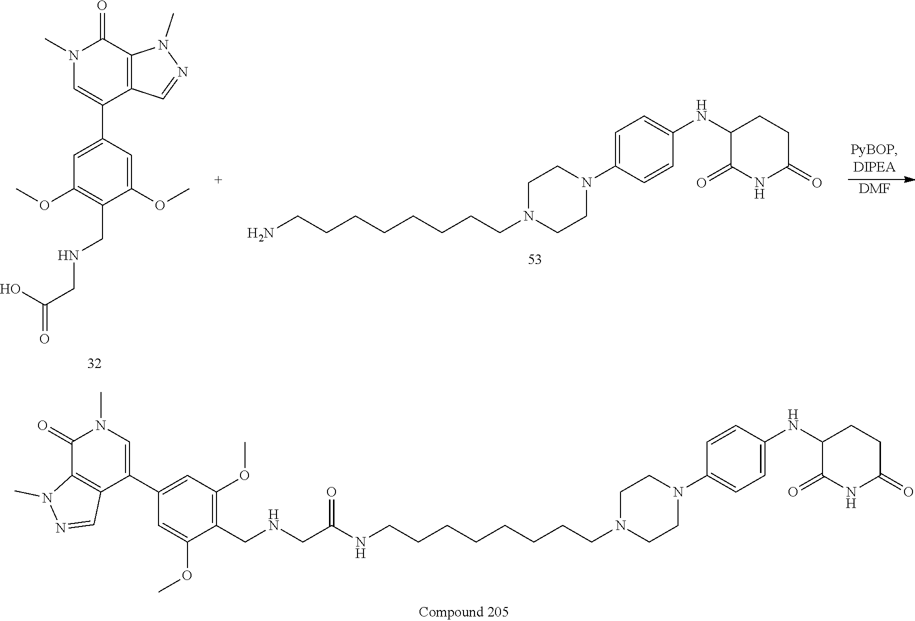

Into a 25 mL single-necked round-bottomed flask containing a well-stirred solution of 2-[[4-(1,6-dimethyl-7-oxo-pyrazolo[3,4-c]pyridin-4-yl)-2,6-dimethoxy-phenyl]methylamino]acetic acid (32, 23.25 mg, 60.16 μmol) and 3-[4-[4-(8-aminooctyl)piperazin-1-yl]anilino]piperidine-2,6-dione (53, 25 mg, 60.16 μmol) in DMF (2 mL) were added DIPEA (23.33 mg, 180.47 μmol, 31.44 uL) and PyBoP (46.96 mg, 90.24 μmol) under nitrogen atmosphere at room temperature. The resulting mixture was stirred at room temperature for 16 hours. The reaction mixture was concentrated and purified by prep-HPLC SUNFIRE OBD C18(100×30)MM 5μ) Mobile phase: A: 0.1% TFA in water B: ACN to afford 4 mg of 2-[[4-(1,6-dimethyl-7-oxo-pyrazolo[3,4-c]pyridin-4-yl)-2,6-dimethoxy-phenyl]methylamino]-N-[8-[4-[4-[(2,6-dioxo-3-piperidyl)amino]phenyl]piperazin-1-yl]octyl]acetamide (Compound 205, 4 mg, 4.94 μmol, 8.22% yield) as a brown liquid. ¹H NMR (400 MHz, Methanol-d₄) δ 7.97 (s, 1H), 7.44 (d, J=1.5 Hz, 1H), 6.94 (s, 2H), 6.91 (d, J=8.9 Hz, 2H), 6.77 (d, J=8.7 Hz, 2H), 4.39 (s, 3H), 4.35 (s, 2H), 4.24 (dd, J=11.8, 5.0 Hz, 1H), 3.98 (d, J=1.4 Hz, 6H), 3.69 (d, J=4.5 Hz, 5H), 3.66-3.55 (m, 3H), 3.26-3.13 (m, 6H), 2.98 (s, 2H), 2.83-2.67 (m, 2H), 2.35-2.26 (m, 1H), 2.17-2.02 (m, 1H), 2.00-1.91 (m, 1H), 1.89-1.84 (m, 1H), 1.81-1.70 (m, 2H), 1.67 (s, 1H), 1.56-1.46 (m, 2H), 1.36 (d, J=13.5 Hz, 8H), 1.32-1.23 (m, 3H).

LCMS (ES+): m/z 784 [M+H]⁺

### Degrader Synthesis 7: Compound 206

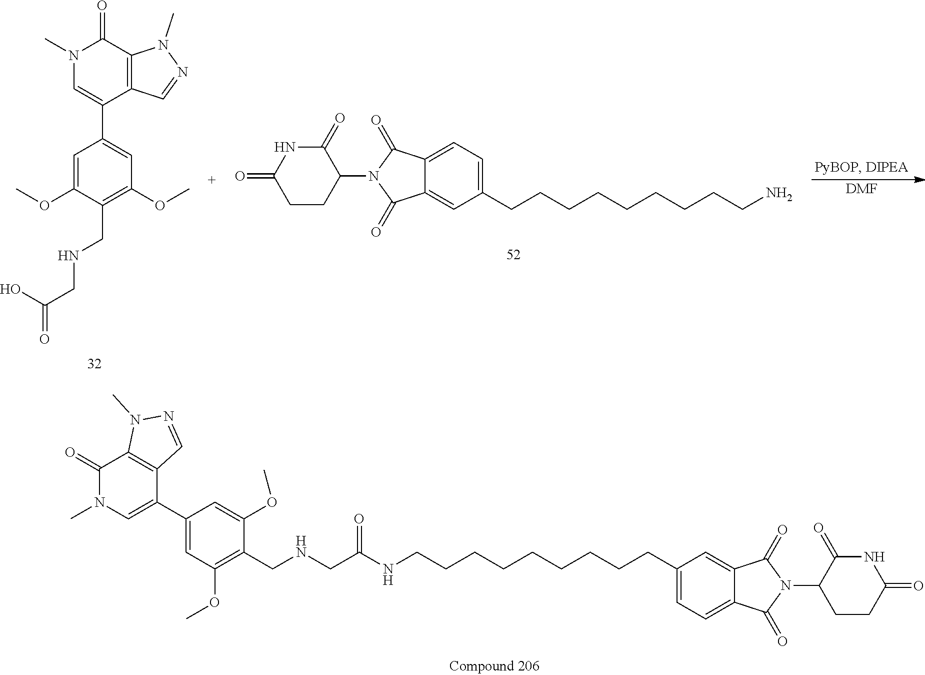

Into a 25 mL single-necked round-bottomed flask containing a well-stirred solution of 2-[[4-(1,6-dimethyl-7-oxo-pyrazolo[3,4-c]pyridin-4-yl)-2,6-dimethoxy-phenyl]methylamino]acetic acid (32, 50 mg, 129.40 μmol) and 5-(9-aminononyl)-2-(2,6-dioxo-3-piperidyl)isoindoline-1,3-dione (52, 51.69 mg, 129.40 μmol) in DMF (5 mL) were added DIPEA (50.17 mg, 388.20 μmol, 67.62 uL) and PyBOP (80.81 mg, 155.28 μmol) under nitrogen atmosphere at room temperature. The resulting mixture was stirred at room temperature for 16 hours. After completion, 20 mL of cold water was added and the reaction was stirred for 10 minutes. The resulting solid was filtered and dried to afford crude product that was purified by reverse phase prep purification (SUNFIRE OBD C18(100×30)MM 5μ) Mobile phase: A: 0.1% TFA in water B: ACN) to obtain 2-[[4-(1,6-dimethyl-7-oxo-pyrazolo[3,4-c]pyridin-4-yl)-2,6-dimethoxy-phenyl]methylamino]-N4942-(2,6-dioxo-3-piperidyl)-1,3-dioxo-isoindolin-5-yl]nonyl]acetamide (Compound 206, 4 mg, 5.21 μmol, 4.03% yield). ¹H NMR (400 MHz, DMSO-d₆) δ 11.12 (s, 1H), 8.09 (d, J=1.6 Hz, 1H), 7.83-7.76 (m, 2H), 7.74 (s, 1H), 7.68 (d, J=7.6 Hz, 1H), 7.55 (d, J=1.6 Hz, 1H), 6.83 (d, J=1.7 Hz, 2H), 5.13 (dd, J=12.7, 5.6 Hz, 1H), 4.31 (d, J=1.7 Hz, 3H), 3.86 (d, J=1.7 Hz, 6H), 3.69 (s, 2H), 3.58 (d, J=1.7 Hz, 3H), 3.10-3.01 (m, 4H), 2.95-2.83 (m, 2H), 2.75 (t, J=7.6 Hz, 2H), 2.64-2.56 (m, 2H), 2.09-2.01 (m, 1H), 1.63-1.53 (m, 2H), 1.43-1.33 (m, 2H), 1.25 (d, J=8.5 Hz, 9H). LCMS (ES+): m/z 768 [M+H]⁺

### Degrader Synthesis 8: Compound 207

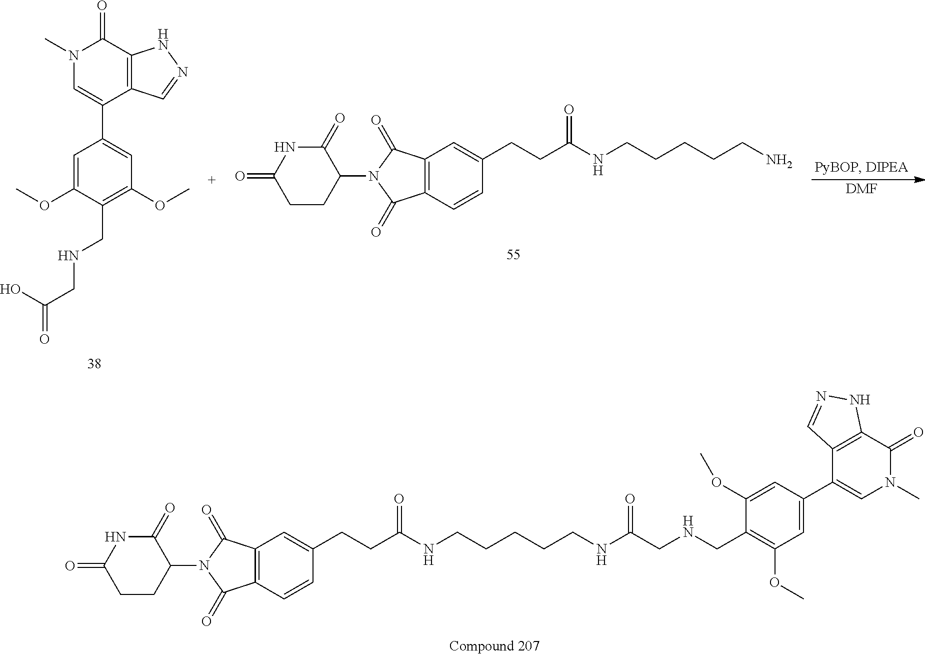

To a stirred solution of 2-[[2,6-dimethoxy-4-(6-methyl-7-oxo-1H-pyrazolo[3,4-c]pyridin-4-yl)phenyl]methylamino]acetic acid (38, 30 mg, 80.56 μmol) in DMF (5 mL), N-(5-aminopentyl)-342-(2,6-dioxo-3-piperidyl)-1,3-dioxo-isoindolin-5-yl]propanamide (55, 50.09 mg, 120.85 μmol) PyBOP (62.89 mg, 120.85 μmol) and DiPEA (52.06 mg, 402.82 μmol, 70.16 uL) was added under nitrogen. The reaction was stirred at room temperature for 16 hours. After reaction completion (monitored by TLC), ice water was added and the reaction mixture was extracted with DCM. The combined organic layer was washed with brine solution, dried over anhydrous sodium sulfate and concentrated under reduced pressure. The crude material was purified by reverse phase prep purification (SUNFIRE OBD C18(100×30)MM 5μ) Mobile phase: A: 0.1% TFA in water B: ACN) to obtain N-[5-[[2-[[2,6-dimethoxy-4-(6-methyl-7-oxo-1H-pyrazolo[3,4-c]pyridin-4 yl)phenyl]methylamino]acetyl]amino]pentyl]-3-[2-(2,6-dioxo-3-piperidyl)-1,3-dioxo-isoindolin-5-yl]propanamide (Compound 207, 5 mg, 6.41 μmol, 7.96% yield) as a white solid. ¹H NMR (400 MHz, DMSO-d₆) δ 11.12 (s, 1H), 8.74 (s, 2H), 8.28 (t, J=5.6 Hz, 1H), 8.19 (s, 1H), 7.85-7.79 (m, 2H), 7.76 (s, 1H), 7.69 (dd, J=7.8, 1.4 Hz, 1H), 7.62 (s, 1H), 6.93 (s, 2H), 5.13 (dd, J=12.8, 5.4 Hz, 1H), 4.14 (t, J=4.8 Hz, 2H), 3.92 (s, 6H), 3.62 (s, 3H), 3.57-3.52 (m, 2H), 3.10 (q, J=6.7 Hz, 2H), 3.00 (t, J=6.9 Hz, 4H), 2.89 (ddd, J=16.6, 13.8, 5.3 Hz, 2H), 2.63-2.54 (m, 1H), 2.44 (t, J=7.4 Hz, 3H), 2.09-1.99 (m, 1H), 1.36 (dp, J=22.4, 7.2 Hz, 5H), 1.27-1.15 (m, 4H). LCMS (ES+): m/z 769 [M+H]⁺

### Degrader Synthesis 9: Compound 208

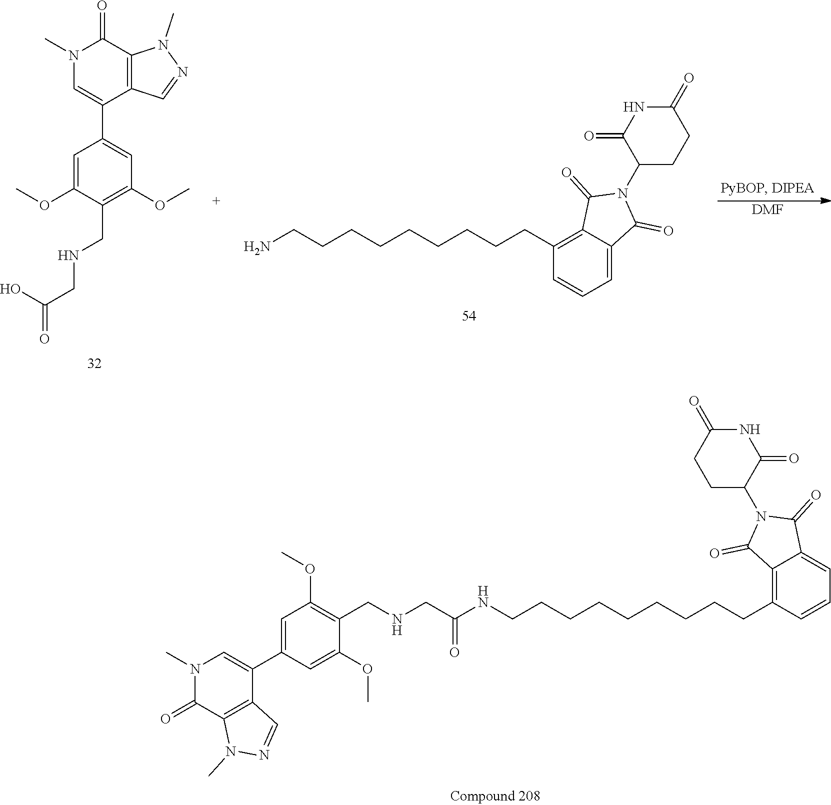

Into a 25 mL single-necked round-bottomed flask containing a well-stirred solution of 2-[[4-(1,6-dimethyl-7-oxo-pyrazolo[3,4-c]pyridin-4-yl)-2,6-dimethoxy-phenyl]methylamino]acetic acid (32, 50 mg, 129.40 μmol) and 4-(9-aminononyl)-2-(2,6-dioxo-3-piperidyl)isoindoline-1,3-dione (54, 51.69 mg, 129.40 μmol) in DMF (5 mL) were added DIPEA (50.17 mg, 388.20 μmol, 67.62 uL) and PyBOP (80.81 mg, 155.28 μmol) under nitrogen atmosphere at room temperature. The resulting mixture was stirred at room temperature for 16 hours. After completion, 20 mL of cold water was added and the reaction was stirred for 10 minutes. The resulting material was filtered and dried to afford crude product that was purified by reverse phase prep purification (SUNFIRE OBD C18(100×30)MM 5μ) Mobile phase: A: 0.1% TFA in water B: ACN) to obtain 2-[[4-(1,6-dimethyl-7-oxo-pyrazolo[3,4-c]pyridin-4-yl)-2,6-dimethoxy-phenyl]methylamino]-N-[9-[2-(2,6-dioxo-3-piperidyl)-1,3-dioxo-isoindolin-4-yl]nonyl]acetamide (Compound 208, 4 mg, 5.21 μmol, 4.03% yield). ¹H NMR (400 MHz, Methanol-d₄) δ 7.97 (s, 1H), 7.70 (s, 1H), 7.69 (d, J=1.6 Hz, 1H), 7.62-7.57 (m, 1H), 7.43 (s, 1H), 6.93 (s, 2H), 5.11 (dd, J=12.6, 5.5 Hz, 1H), 4.38 (s, 3H), 4.34 (s, 2H), 3.98 (s, 6H), 3.68 (d, J=2.7 Hz, 4H), 3.21 (t, J=7.1 Hz, 2H), 3.10-3.03 (m, 2H), 2.93-2.80 (m, 1H), 2.80-2.65 (m, 2H), 2.12 (dtd, J=12.9, 4.9, 2.3 Hz, 1H), 1.63 (dt, J=15.2, 7.3 Hz, 3H), 1.53-1.43 (m, 2H), 1.39-1.26 (m, 8H). LCMS (ES+): m/z 768 [M+H]⁺

### Degrader Synthesis 10: Compound 209

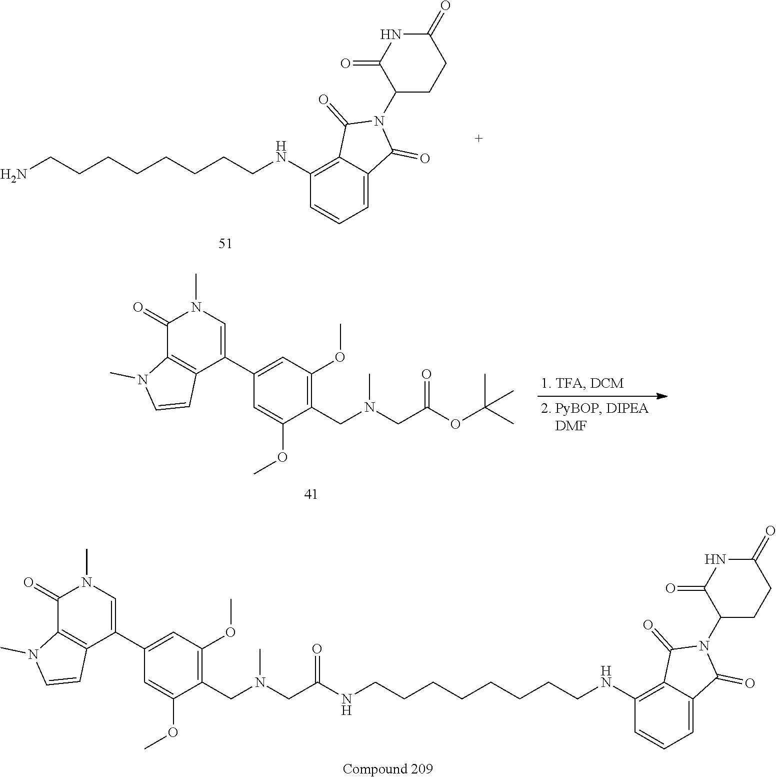 [SMILES: NCCCCCCCCNc1cccc2c1C(=O)N(C1CCC(=O)NC1=O)C2=O ; COc1cc(-c2cn(C)c(=O)c3c2ccn3C)cc(OC)c1CN(C)CC(=O)OC(C)(C)C ; COc1cc(-c2cn(C)c(=O)c3c2ccn3C)cc(OC)c1CN(C)CC(=O)NCCCCCCCCNc1cccc2c1C(=O)N(C1CCC(=O)NC1=O)C2=O]

Into a 25 mL single-necked round-bottomed flask containing a well-stirred solution of tert-butyl 2-[[4-(1,6-dimethyl-7-oxo-pyrrolo[2,3-c]pyridin-4-yl)-2,6-dimethoxy-phenyl]methyl-methyl-amino]acetate (41, 51.71 mg, 113.51 μmol) in anhydrous DCM (4 mL) was added TFA (64.71 mg, 567.55 μmol, 43.72 uL) under nitrogen atmosphere at room temperature. The resulting mixture was stirred at room temperature for 30 minutes before the mixture was concentrated under reduced pressure. The resulting material was dissolved in DMF (3 mL) and 4-(8-aminooctylamino)-2-(2,6-dioxo-3-piperidyl)isoindoline-1,3-dione (51, 50 mg, 124.85 μmol), DIPEA (44.01 mg, 340.53 μmol, 59.31 uL) and PyBOP (88.60 mg, 170.27 μmol) were added. The reaction was stirred under nitrogen atmosphere at room temperature for 16 hours. The reaction mixture was concentrated under reduced pressure and purified by prep-HPLC SUNFIRE OBD C18(100×30)MM 5μ) Mobile phase: A: 0.1% TFA in water B: ACN to afford 2-[[4-(1,6-dimethyl-7-oxo-pyrrolo[2,3-c]pyridin-4-yl)-2,6-dimethoxy-phenyl]methyl-methyl-amino]-N-[8-[[2-(2,6-dioxo-3-piperidyl)-1,3-dioxo-isoindolin-4-yl]amino]octyl]acetamide (Compound 209, 39 mg, 47.48 μmol, 41.83% yield) as a yellow gummy liquid. LCMS (ES+): m/z 782 [M+H]⁺

### Degrader Synthesis 11: Compound 210

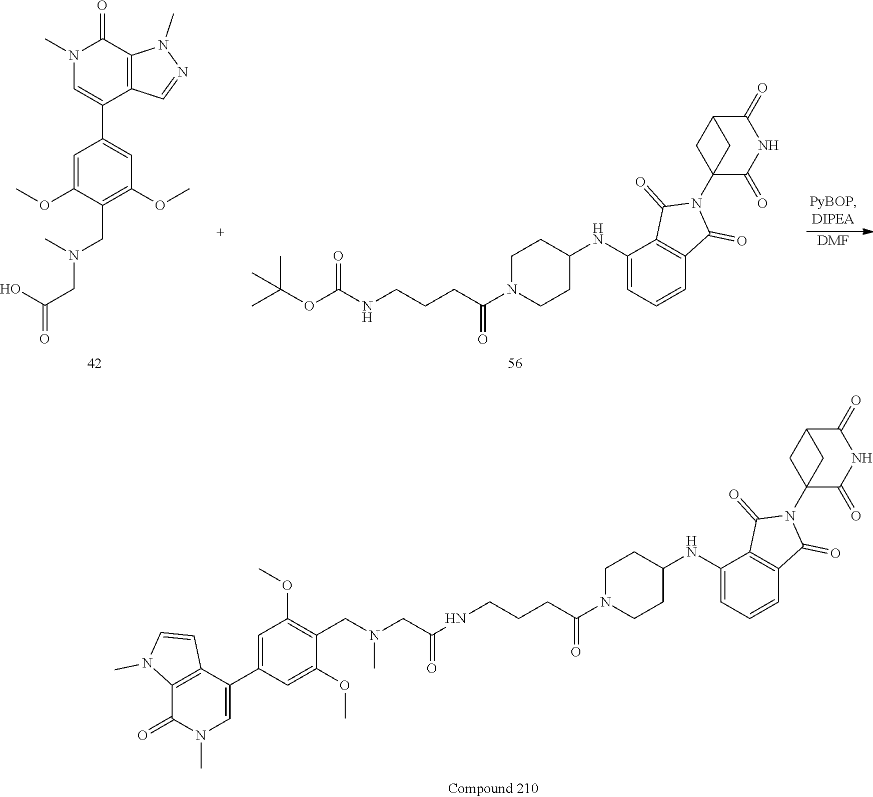

Into a 25 mL single-necked round-bottomed flask containing a well-stirred solution of tert-butyl N-[4-[4-[[2-(2,4-dioxo-3-azabicyclo[3.1.1]heptan-5-yl)-1,3-dioxo-isoindolin-4-yl]amino]-1-piperidyl]-4-oxo-butyl]carbamate (56, 53.36 mg, 96.39 μmol) in anhydrous DCM (5 mL) was added TFA (49.96 mg, 438.11 μmol, 33.75 uL) under nitrogen atmosphere at room temperature. The resulting mixture was stirred at room temperature for 30 minutes before the mixture was concentrated under reduced pressure. The resulting material was dissolved in DMF (4 mL) and 2-[[4-(1,6-dimethyl-7-oxo-pyrrolo[2,3-c]pyridin-4-yl)-2,6-dimethoxy-phenyl]methyl-methyl-amino]acetic acid (42, 35 mg, 87.62 μmol) and DIPEA (33.97 mg, 262.87 μmol, 45.79 uL) were added. The reaction was stirred under a nitrogen atmosphere at room temperature for 16 hours. The reaction mixture was concentrated under reduced pressure and purified by prep-HPLC SUNFIRE OBD C18(100×30)MM 5μ) Mobile phase: A: 0.1% TFA in water B: ACN to afford 2-[[4-(1,6-dimethyl-7-oxo-pyrrolo[2,3-c]pyridin-4-yl)-2,6-dimethoxy-phenyl]methyl-methyl-amino]-N-[4-[4-[[2-(2,4-dioxo-3-azabicyclo[3.1.1]heptan-5-yl)-1,3-dioxo-isoindolin-4-yl]amino]-1-piperidyl]-4-oxo-butyl]acetamide (Compound 210, 23 mg, 27.47 μmol, 31.34% yield) as a yellow gummy liquid. ¹H NMR (400 MHz, DMSO-d₆) δ 11.08 (s, 1H), 9.47 (s, 1H), 8.43 (s, 1H), 7.59 (t, J=7.8 Hz, 1H), 7.50 (s, 1H), 7.38 (d, J=2.9 Hz, 1H), 7.22 (d, J=8.7 Hz, 1H), 7.03 (d, J=7.0 Hz, 1H), 6.89 (s, 2H), 4.34-4.24 (m, 1H), 4.10 (s, 3H), 3.88 (s, 6H), 3.82-3.77 (m, 3H), 3.55 (s, 2H), 3.21-3.08 (m, 4H), 3.00-2.87 (m, 4H), 2.83-2.64 (m, 4H), 2.39-2.30 (m, 2H), 1.94 (s, 2H), 1.72-1.61 (m, 2H), 1.48-1.35 (m, 1H), 1.35-1.21 (m, 1H). LCMS (ES+): m/z 835 [M+H]⁺

### Degrader Synthesis 12: Compound 211

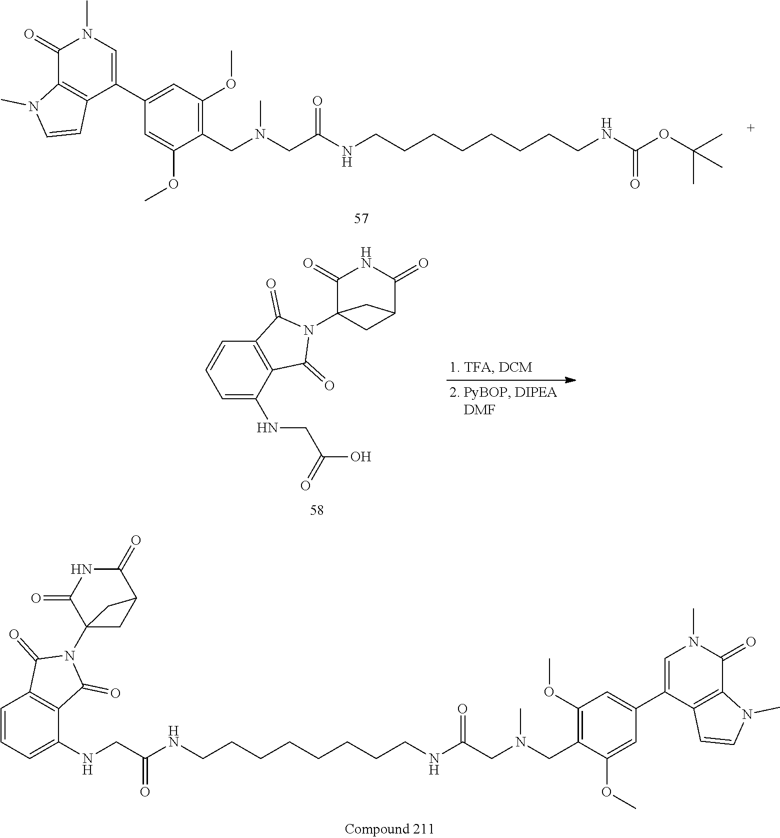 [SMILES: COc1cc(-c2cn(C)c(=O)c3c2ccn3C)cc(OC)c1CN(C)CC(=O)NCCCCCCCCNC(=O)OC(C)(C)C ; O=C(O)CNc1cccc2c1C(=O)N(C13CC(C1)C(=O)NC3=O)C2=O ; COc1cc(-c2cn(C)c(=O)c3c2ccn3C)cc(OC)c1CN(C)CC(=O)NCCCCCCCCNC(=O)CNc1cccc2c1C(=O)N(C13CC(C1)C(=O)NC3=O)C2=O]

To a 25 mL single-necked round-bottomed flask containing a well-stirred solution of tert-butyl N-[8-[[2-[[4-(1,6-dimethyl-7-oxo-pyrrolo[2,3-c]pyridin-4-yl)-2,6-dimethoxy-phenyl]methyl-methyl-amino]acetyl]amino]octyl]carbamate (57, 85.93 mg, 137.31 μmol) in anhydrous DCM (5 mL) was added TFA (78.28 mg, 686.53 μmol, 52.89 uL) under nitrogen atmosphere at room temperature. The resulting mixture was stirred at room temperature for 30 minutes and was subsequently concentrated under reduced pressure. The material was subsequently dissolved in DMF (4 mL) and 2-[[2-(2,4-dioxo-3-azabicyclo[3.1.1]heptan-5-yl)-1,3-dioxo-isoindolin-4-yl]amino]acetic acid (58, 47.14 mg, 137.31 μmol), DIPEA (53.24 mg, 411.92 μmol, 71.75 uL) and PyBOP (107.18 mg, 205.96 μmol) were added. The reaction was stirred under a nitrogen atmosphere at room temperature for 16 hours. The reaction mixture was concentrated under reduced pressure and purified by prep-HPLC SUNFIRE OBD C18(100×30)MM 50 Mobile phase: A: 0.1% TFA in water B: ACN to afford N-[8-[[2-[[4-(1,6-dimethyl-7-oxo-pyrrolo[2,3-c]pyridin-4-yl)-2,6-dimethoxy-phenyl]methyl-methyl-amino]acetyl]amino]octyl]-2-[[2-(2,4-dioxo-3-azabicyclo[3.1.1]heptan-5-yl)-1,3-dioxo-isoindolin-4-yl]amino]acetamide (Compound 211, 15 mg, 16.82 μmol, 12.25% yield) as a yellow solid. ¹H NMR (400 MHz, DMSO-d₆) δ 11.07 (d, J=2.2 Hz, 1H), 9.45 (s, 1H), 8.37 (t, J=5.2 Hz, 1H), 8.06 (t, J=5.6 Hz, 1H), 7.57 (dd, J=8.5, 7.2 Hz, 1H), 7.50 (s, 1H), 7.39 (d, J=2.8 Hz, 1H), 7.03 (d, J=7.1 Hz, 1H), 6.94-6.88 (m, 3H), 6.84 (d, J=8.5 Hz, 1H), 6.48 (d, J=2.8 Hz, 1H), 4.31 (d, J=3.3 Hz, 2H), 4.11 (s, 2H), 3.89 (s, 7H), 3.80 (d, J=4.9 Hz, 2H), 3.55 (s, 3H), 3.17-3.04 (m, 6H), 3.00-2.90 (m, 4H), 2.72 (d, J=4.7 Hz, 3H), 1.44-1.34 (m, 4H), 1.22 (s, 8H). LCMS (ES+): m/z 851 [M+H]⁺

### Degrader Synthesis 13: Compound 212

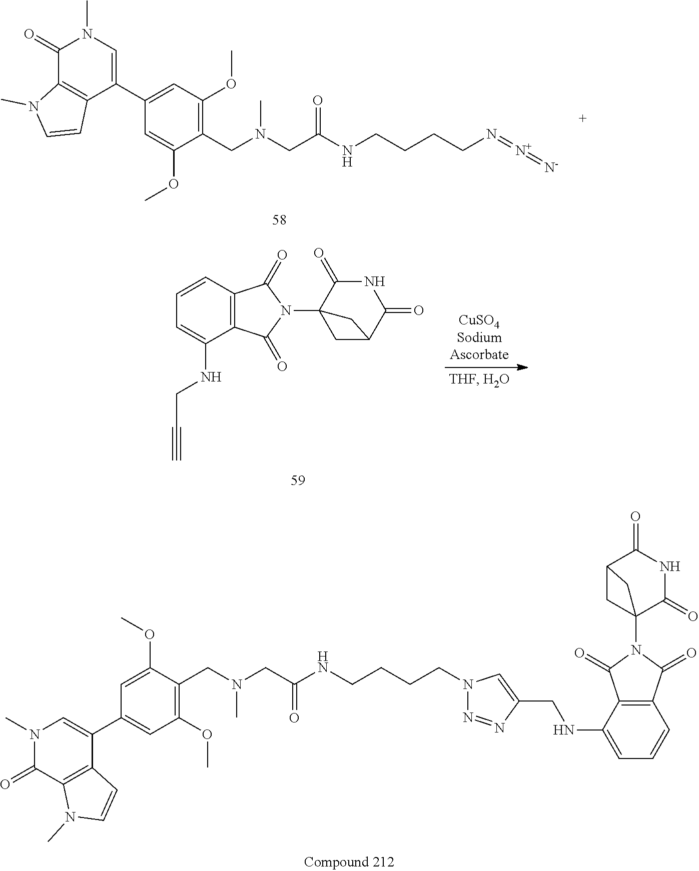 [SMILES: COc1cc(-c2cn(C)c(=O)c3c2ccn3C)cc(OC)c1CN(C)CC(=O)NCCCCN=[N+]=[N-] ; C#CCNc1cccc2c1C(=O)N(C13CC(C1)C(=O)NC3=O)C2=O ; COc1cc(-c2cn(C)c(=O)c3c2ccn3C)cc(OC)c1CN(C)CC(=O)NCCCCn1cc(CNc2cccc3c2C(=O)N(C24CC(C2)C(=O)NC4=O)C3=O)nn1]

Into a 25 mL single-necked round-bottomed flask containing a well-stirred solution of N-(4-azidobutyl)-2-[[4-(1,6-dimethyl-7-oxo-pyrrolo[2,3-c]pyridin-4-yl)-2,6-dimethoxy-phenyl]methyl-methyl-amino]acetamide (58, 70 mg, 141.25 μmol) in THF (2 mL) and water (2 mL) were added 2-(2,4-dioxo-3-azabicyclo[3.1.1]heptan-5-yl)-4-(prop-2-ynylamino)isoindoline-1,3-dione (59, 45.67 mg, 141.25 μmol), copper(II) sulfate (45.09 mg, 282.50 μmol, 12.53 uL) and sodium ascorbate (55.97 mg, 282.50 μmol) at room temperature. The resulting mixture was stirred at room temperature for 16 hours. The reaction mixture was filtered through a pad of Celite® and concentrated under pressure. The crude material was purified by prep-HPLC SUNFIRE OBD C18(100×30)MM 5μ) Mobile phase: A: 0.1% TFA in water B: ACN to afford 2-[[4-(1,6-dimethyl-7-oxo-pyrrolo2-[[4-(1,6-dimethyl-7-oxo-pyrrolo[2,3-c]pyridin-4-yl)-2,6-dimethoxy-phenyl]methyl-methyl-amino]-N-[4-[4-[[[2-(2,4-dioxo-3-azabicyclo[3.1.1]heptan-5-yl)-1,3-dioxo-isoindolin-4-yl]amino]methyl]triazol-1-yl]butyl]acetamide (Compound 212, 23 mg, 26.85 μmol, 19.01% yield) as a yellow solid. ¹H NMR (400 MHz, DMSO-d₆) δ 11.08 (s, 1H), 9.49 (s, 1H), 8.42 (s, 1H), 8.00 (d, J=2.9 Hz, 1H), 7.60-7.53 (m, 1H), 7.50 (d, J=2.8 Hz, 1H), 7.39 (t, J=2.9 Hz, 1H), 7.24-7.08 (m, 2H), 7.07-6.96 (m, 2H), 6.89 (d, J=2.8 Hz, 2H), 6.48 (t, J=2.9 Hz, 1H), 4.57 (d, J=5.4 Hz, 2H), 4.37-4.27 (m, 4H), 4.11 (d, J=2.8 Hz, 3H), 3.87 (d, J=2.8 Hz, 6H), 3.79 (s, 2H), 3.55 (d, J=2.8 Hz, 3H), 3.11 (s, 4H), 2.93 (s, 4H), 2.70 (s, 3H), 1.85-1.74 (m, 2H), 1.43-1.33 (m, 2H). LCMS (ES+): m/z 819 [M+H]⁺

### Degrader Synthesis 14: Compound 213

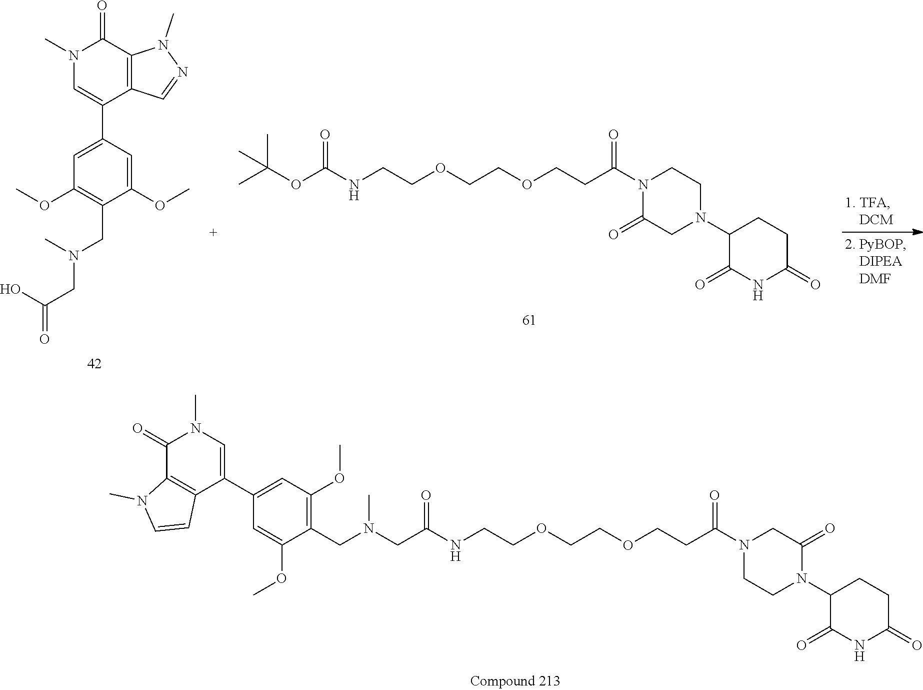

Into a 25 mL single-necked round-bottomed flask containing a well-stirred solution of tert-butyl N-[2-[2-[3-[4-(2,6-dioxo-3-piperidyl)-2-oxo-piperazin-1-yl]-3-oxo-propoxy]ethoxy]ethyl]carbamate (61, 132.52 mg, 281.65 μmol) in anhydrous DCM (5 mL) was added TFA (107.05 mg, 938.82 μmol, 72.33 uL) under nitrogen atmosphere at room temperature. The resulting mixture was stirred at room temperature for 30 minutes. The reaction mixture was concentrated under reduced pressure and the resulting material was dissolved in DMF (4 mL) and 24-[4-(1,6-dimethyl-7-oxo-pyrrolo[2,3-c]pyridin-4-yl)-2,6-dimethoxy-phenyl]methyl-methyl-amino]acetic acid (42, 75 mg, 187.76 μmol), DIPEA (72.80 mg, 563.29 μmol, 98.11 uL) and PyBOP (146.57 mg, 281.65 μmol) were added under a nitrogen atmosphere at room temperature for 16 hours. The reaction mixture was concentrated under reduced pressure and purified by prep-HPLC SUNFIRE OBD C18(100×30)MM 5μ) Mobile phase: A: 0.1% TFA in water B: ACN to afford 2-[[4-(1,6-dimethyl-7-oxo-pyrrolo[2,3-c]pyridin-4-yl)-2,6-dimethoxy-phenyl]methyl-methyl-amino]-N-[2-[2-[3-[4-(2,6-dioxo-3-piperidyl)-3-oxo-piperazin-1-yl]-3-oxo-propoxy]ethoxy]ethyl]acetamide (Compound 213, 9 mg, 11.17 μmol, 5.95% yield) as a yellow solid. LCMS (ES+): m/z 752 [M+H]⁺

### Degrader Synthesis 15: Compound 214

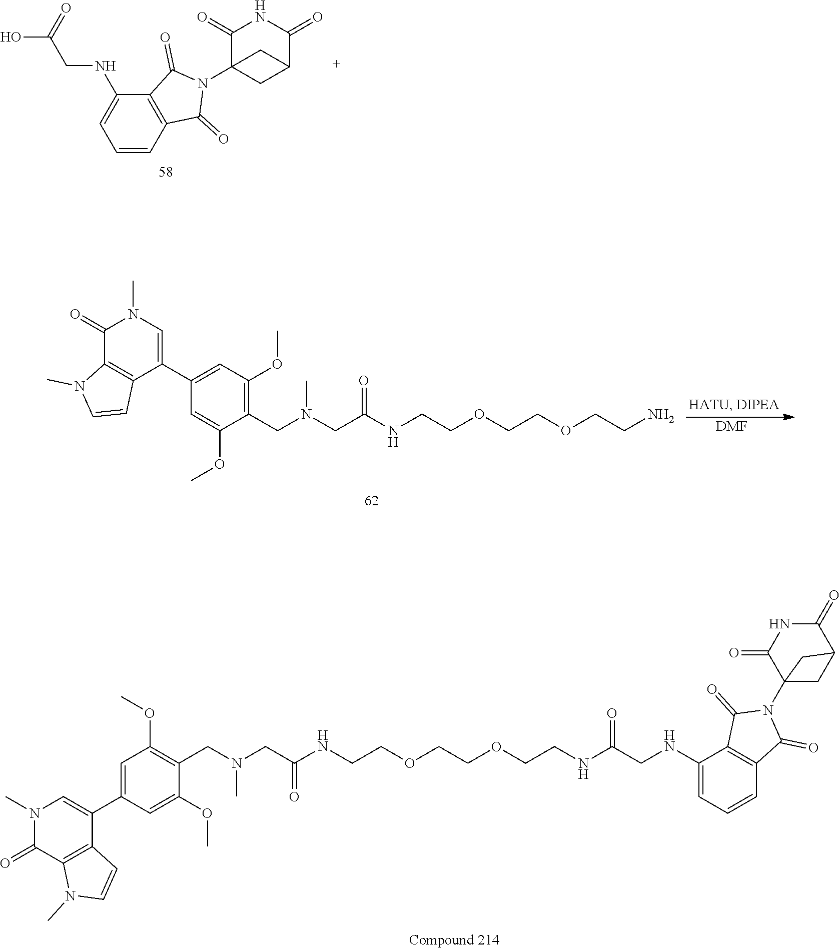

An oven dried round bottom flask was charged with a solution of 2-[[2-(2,4-dioxo-3-azabicyclo[3.1.1]heptan-5-yl)-1,3-dioxo-isoindolin-4-yl]amino]acetic acid (58, 30 mg, 87.39 μmol) in DMF (3 mL) and N-[2-[2-(2-aminoethoxy)ethoxy]ethyl]-24-[4-(1,6-dimethyl-7-oxo-pyrrolo[2,3-c]pyridin-4-yl)-2,6-dimethoxy-phenyl]methyl-methyl-amino]acetamide (62, 45.06 mg, 85.07 μmop, DIPEA (54.97 mg, 425.35 μmol, 74.09 uL) and HATU (38.82 mg, 102.08 μmol) were added. The reaction mixture was stirred for 16 hours at room temperature. The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to afford N-[2-[2-[2-[[2-[[4-(1,6-dimethyl-7-oxo-pyrrolo[2,3-c]pyridin-4-yl)-2,6-dimethoxy-phenyl]methyl-methyl-amino]acetyl]amino]ethoxy]ethoxy]ethyl]-2-[[2-(2,4-dioxo-3-azabicyclo[3.1.1]heptan-5-yl)-1,3-dioxo-isoindolin-4-yl]amino]acetamide (Compound 214, 4.01 mg, 4.33 μmol, 5.09% yield) as a yellow solid. ¹H NMR (400 MHz, Methanol-d₄) δ 7.39 (s, 1H), 7.30 (d, J=2.9 Hz, 1H), 6.93 (d, J=1.4 Hz, 2H), 6.51 (d, J=2.9 Hz, 1H), 4.50 (d, J=2.2 Hz, 2H), 4.29 (d, J=8.0 Hz, 1H), 4.18 (s, 4H), 3.97 (s, 8H), 3.92-3.84 (m, 2H), 3.75 (q, J=5.9 Hz, 3H), 3.67 (s, 3H), 3.58 (d, J=4.7 Hz, 4H), 3.52 (t, J=5.3 Hz, 2H), 3.40 (t, J=5.2 Hz, 2H), 2.92 (d, J=3.2 Hz, 3H), 2.69 (ddt, J=28.0, 16.1, 5.6 Hz, 5H), 2.46-2.28 (m, 1H), 2.05-1.96 (m, 1H). LCMS (ES+): m/z 782 [M+H]⁺

### Degrader Synthesis 16: Compound 215

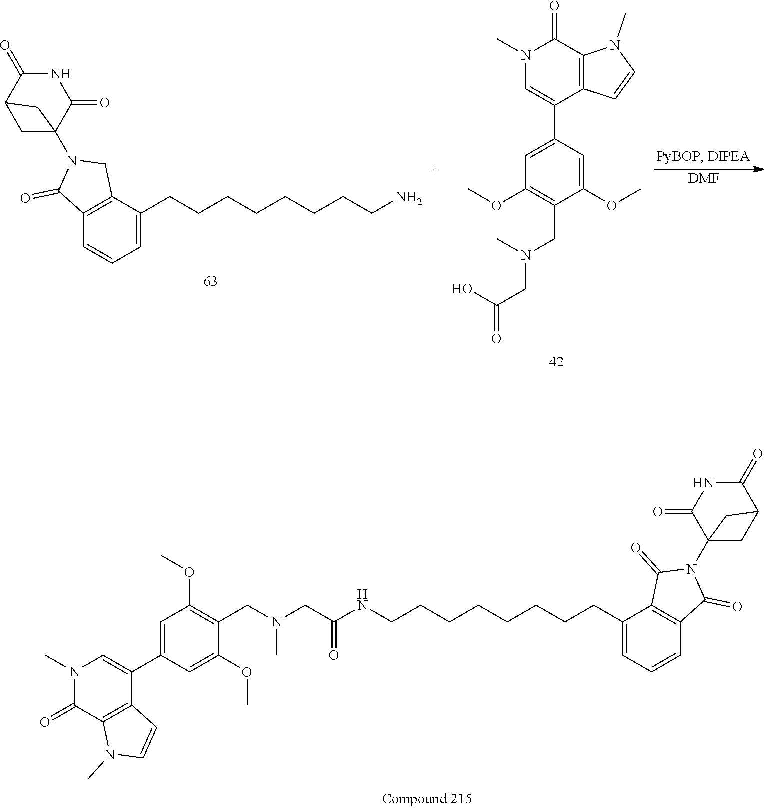

Into a 25 mL single-necked round-bottomed flask containing a well-stirred solution of 5-[4-(8-aminooctyl)-1-oxo-isoindolin-2-yl]-3-azabicyclo[3.1.1]heptane-2,4-dione (63, 40 mg, 104.31 μmol) and 2-[[4-(1,6-dimethyl-7-oxo-pyrrolo[2,3-c]pyridin-4-yl)-2,6-dimethoxy-phenyl]methyl-methyl-amino]acetic acid (42, 41.66 mg, 104.31 μmol) in DMF (5 mL) were added DIPEA (40.44 mg, 312.92 μmol, 54.50 uL) and PyBOP (81.42 mg, 156.46 μmol) under a nitrogen atmosphere at room temperature, the resulting mixture was stirred at room temperature for 16 hours. After completion, 20 mL of cold water was added and the reaction was stirred for 10 minutes. The resulting solid was filtered and dried to afford crude product that was purified by reverse phase prep purification (SUNFIRE OBD C18(100×30)MM 5μ) Mobile phase: A: 0.1% TFA in water B: ACN) to obtain 2-[[4-(1,6-dimethyl-7-oxo-pyrrolo[2,3-c]pyridin-4-yl)-2,6-dimethoxy-phenyl]methyl-methyl-amino]-N-[8-[2-(2,4-dioxo-3-azabicyclo[3.1.1]heptan-5-yl)-1-oxo-isoindolin-4-yl]octyl]acetamide (Compound 215, 18 mg, 23.53 μmol, 22.56% yield). LCMS (ES+): m/z 782 [M+H]⁺

### Degrader Synthesis 17: Compound 216

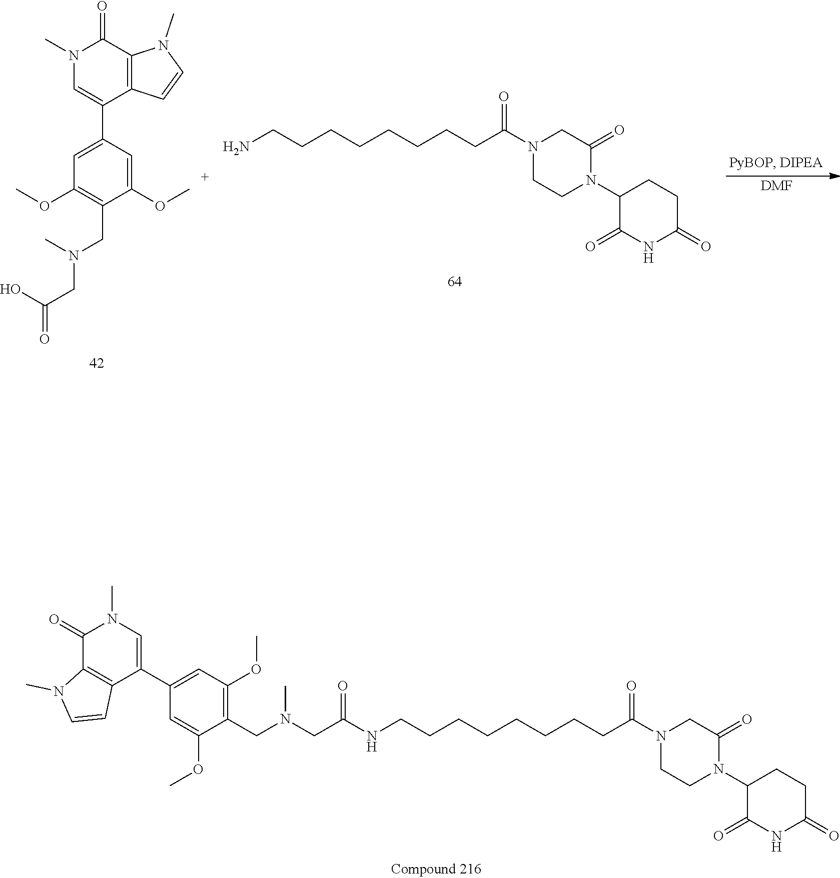

To a stirred solution of 2-[[4-(1,6-dimethyl-7-oxo-pyrrolo[2,3-c]pyridin-4-yl)-2,6-dimethoxy-phenyl]methyl-methyl-amino]acetic acid (42, 70 mg, 175.25 μmol) and 3-[4-(9-aminononanoyl)-2-oxo-piperazin-1-yl]piperidine-2,6-dione (64, 77.06 mg, 210.29 μmol) in DMF (3 mL) was added N,N-diisopropylethylamine (67.95 mg, 525.74 μmol, 91.57 uL) followed by PyBOP (136.79 mg, 262.87 μmol). The reaction mixture was stirred at room temperature for 16 hours. Water was added and the reaction mixture was extracted with ethyl acetate. The combined organics were dried over anhydrous sodium sulfate and concentrated to afford crude material that was purified by preparative HPLC to yield 2-[[4-(1,6-dimethyl-7-oxo-pyrrolo[2,3-c]pyridin-4-yl)-2,6-dimethoxy-phenyl]methyl-methyl-amino]-N-[9-[4-(2,6-dioxo-3-piperidyl)-3-oxo-piperazin-1-yl]-9-oxo-nonyl]acetamide (Compound 216, 5 mg, 6.16 μmol, 3.51% yield). ¹H NMR (400 MHz, Methanol-d₄) δ 8.26 (t, J=5.5 Hz, 1H), 7.37 (d, J=2.5 Hz, 1H), 7.29 (d, J=2.8 Hz, 1H), 6.93 (d, J=0.9 Hz, 2H), 6.50 (dd, J=2.8, 1.0 Hz, 1H), 4.86 (s, 3H), 4.49 (d, J=3.7 Hz, 2H), 4.27-4.24 (m, 1H), 4.20-4.16 (m, 4H), 3.99-3.94 (m, 6H), 3.92-3.84 (m, 2H), 3.67 (d, J=0.9 Hz, 3H), 3.54-3.36 (m, 3H), 3.24-3.10 (m, 2H), 2.93 (d, J=2.7 Hz, 3H), 2.81-2.65 (m, 2H), 2.46-2.31 (m, 3H), 2.09-1.99 (m, 1H), 1.59-1.51 (m, 2H), 1.48-1.40 (m, 2H), 1.27 (s, 7H). LCMS (ES+): m/z 782 [M+H]⁺

### Degrader Synthesis 18: Compound 217

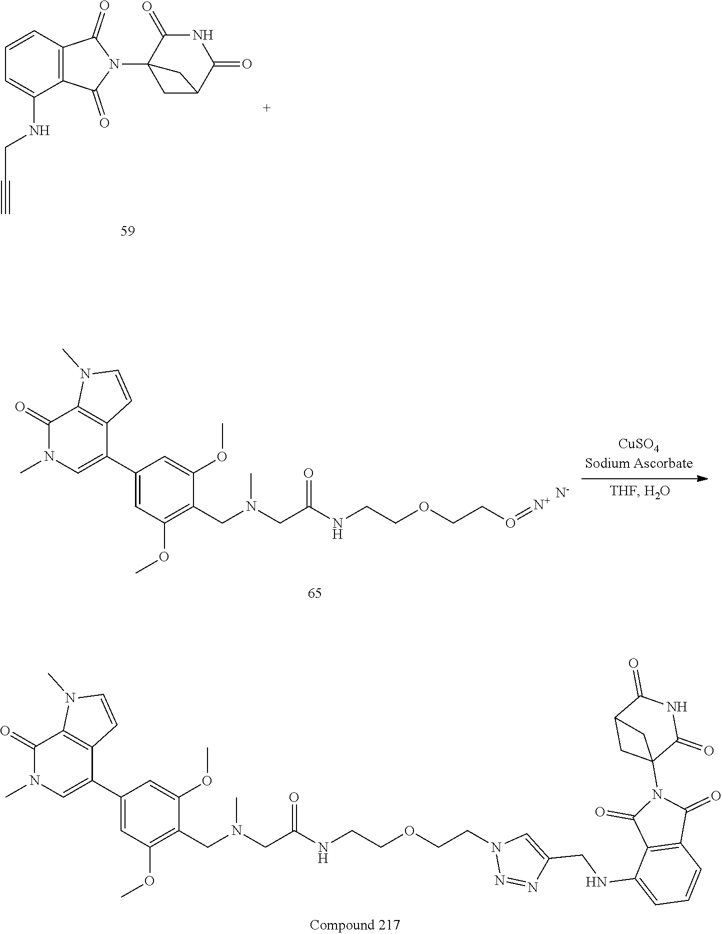

A round bottom flask was charged with a solution of 2-(2,4-dioxo-3-azabicyclo[3.1.1]heptan-5-yl)-4-(prop-2-ynylamino)isoindoline-1,3-dione (59, 30 mg, 92.79 μmol) in THF (4 mL) and water (1 mL). N-[2-(2-Azidoethoxy)ethyl]-2-[[4-(1,6-dimethyl-7-oxo-pyrrolo[2,3-c]pyridin-4-yl)-2,6-dimethoxy-phenyl]methyl-methyl-amino]acetamide (65, 47.47 mg, 92.79 μmol), copper(II) sulfate (29.62 mg, 185.58 μmol, 8.23 uL) and sodium ascorbate (36.77 mg, 185.58 μmol) were added at room temperature. The reaction mixture was stirred for 16 hours at room temperature and the reaction mixture was filtered through Celite®. The filtrate was concentrated under reduced pressure. The crude mixture was purified by reverse phase column chromatography (Column: SUNFIRE OBD C18(100×30)MM 5p), Mobile phase: A: 0.1% TFA in water, B: ACN) to yield 2-[[4-(1,6-dimethyl-7-oxo-pyrrolo[2,3-c]pyridin-4-yl)-2,6-dimethoxy-phenyl]methyl-methyl-amino]-N-[2-[2-[4-[[[2-(2,4-dioxo-3-azabicyclo[3.1.1]heptan-5-yl)-1,3-dioxo-isoindolin-4-yl]amino]methyl]triazol-1-yl]ethoxy]ethyl]acetamide as a yellow solid (Compound 217, 10.71 mg, 12.33 μmol, 13.28% yield). ¹H NMR (400 MHz, DMSO-d₆) δ 11.07 (d, J=2.2 Hz, 1H), 9.52 (s, 1H), 8.48 (t, J=5.5 Hz, 1H), 8.00 (s, 1H), 7.55 (t, J=7.9 Hz, 1H), 7.49 (s, 1H), 7.38 (d, J=2.9 Hz, 1H), 7.14 (d, J=8.6 Hz, 1H), 7.07-6.98 (m, 2H), 6.89 (s, 2H), 6.48 (d, J=2.9 Hz, 1H), 4.54 (d, J=5.9 Hz, 2H), 4.49 (t, J=5.3 Hz, 2H), 4.31 (s, 2H), 4.11 (s, 3H), 3.87 (s, 6H), 3.82-3.76 (m, 4H), 3.55 (s, 3H), 3.32-3.21 (m, 3H), 3.14-3.08 (m, 1H), 2.98-2.86 (m, 5H), 2.70 (d, J=4.8 Hz, 3H). LCMS (ES+): m/z 782 [M+H]⁺

### Degrader Synthesis 19: Compound 218

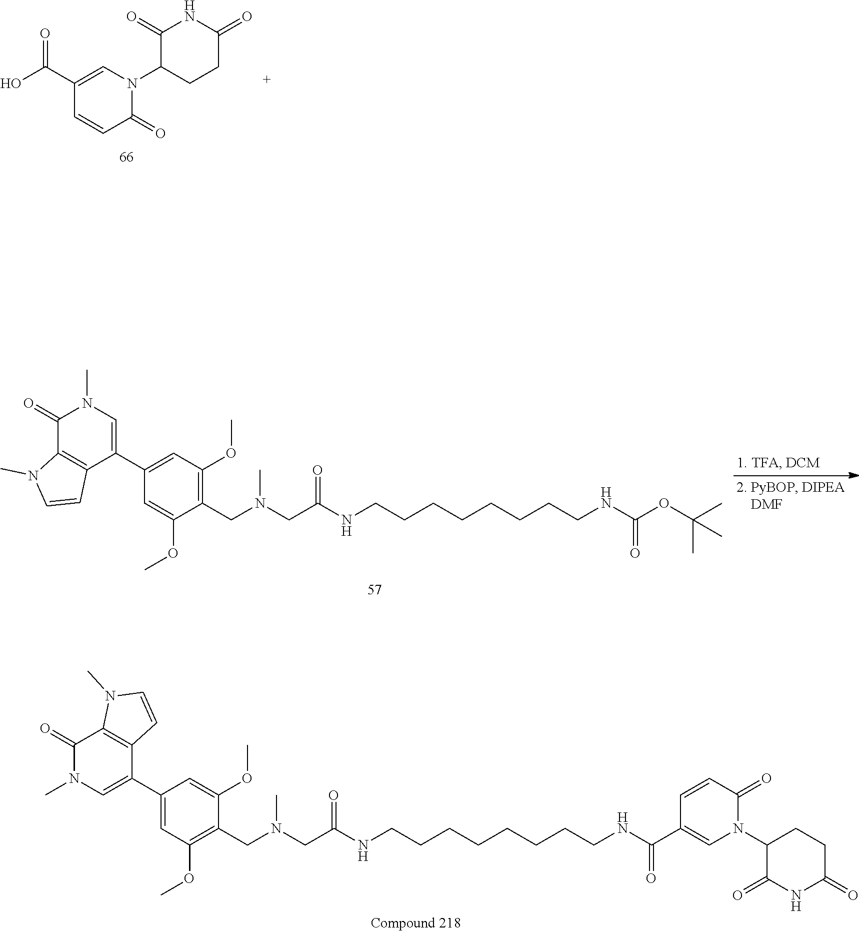 [SMILES: O=C1CCC(n2cc(C(=O)O)ccc2=O)C(=O)N1 ; COc1cc(-c2cn(C)c(=O)c3c2ccn3C)cc(OC)c1CN(C)CC(=O)NCCCCCCCCNC(=O)OC(C)(C)C ; COc1cc(-c2cn(C)c(=O)c3c2ccn3C)cc(OC)c1CN(C)CC(=O)NCCCCCCCCNC(=O)c1ccc(=O)n(C2CCC(=O)NC2=O)c1]

Into a 25 mL single-necked round-bottomed flask containing a well-stirred solution of tert-butyl N-[8-[[2-[[4-(1,6-dimethyl-7-oxo-pyrrolo[2,3-c]pyridin-4-yl)-2,6-dimethoxy-phenyl]methyl-methyl-amino]acetyl]amino]octyl]carbamate (57, 65 g, 103.87 mmol) in anhydrous DCM (3 mL) was added TFA (59.22 g, 519.34 mmol, 40.01 mL) under a nitrogen atmosphere at room temperature. The resulting mixture was stirred at room temperature for 30 minutes. The resulting mixture was concentrated under reduced pressure and dissolved in DMF (4 mL). 1-(2,6-Dioxo-3-piperidyl)-6-oxo-pyridine-3-carboxylic acid (66, 25.99 g, 103.87 mmol), DIPEA (40.27 g, 311.60 mmol, 54.28 mL) and PyBOP (81.08 g, 155.80 mmol) were added under a nitrogen atmosphere. The reaction was stirred at room temperature for 16 hours. The reaction mixture was concentrated under reduced pressure and purified by prep-HPLC SUNFIRE OBD C18(100×30) MM Mobile phase: A: 0.1% TFA in water B: ACN to afford N-[8-[[2-[[4-(1,6-dimethyl-7-oxo-pyrrolo[2,3-c]pyridin-4-yl)-2,6-dimethoxy-phenyl]methyl-methyl-amino]acetyl]amino]octyl]-1-(2,6-dioxo-3-piperidyl)-6-oxo-pyridine-3-carboxamide (Compound 218, 7 mg, 8.14 μmol, 7.83% yield) as a colorless gummy liquid. ¹H NMR (400 MHz, Methanol-d₄) δ 8.26 (d, J=2.6 Hz, 1H), 7.95 (dd, J=9.5, 2.5 Hz, 1H), 7.39 (s, 1H), 7.31 (d, J=2.9 Hz, 1H), 6.94 (s, 2H), 6.57 (d, J=9.6 Hz, 1H), 6.52 (d, J=2.9 Hz, 1H), 4.51 (d, J=3.6 Hz, 2H), 4.20 (s, 3H), 3.99 (s, 6H), 3.90 (d, J=20.8 Hz, 2H), 3.68 (s, 3H), 3.25-3.10 (m, 2H), 3.00 (s, 2H), 2.95 (s, 3H), 2.90-2.69 (m, 3H), 2.26-2.17 (m, 1H), 1.69-1.61 (m, 1H), 1.61-1.51 (m, 2H), 1.42 (d, J=23.4 Hz, 5H), 1.29 (s, 8H). LCMS (ES+): m/z 758 [M+H]⁺

### Degrader Synthesis 20: Compound 219

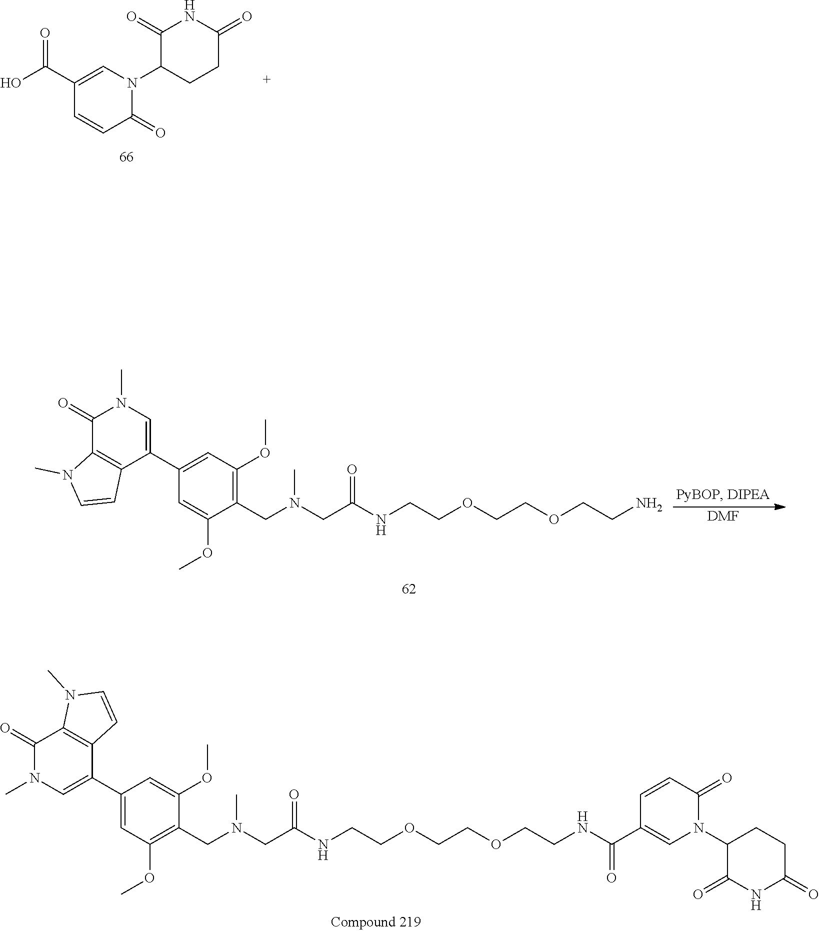

Into a 25 mL single-necked round-bottomed flask containing a well-stirred solution of N-[2-[2-(2-aminoethoxy)ethoxy]ethyl]-2-[[4-(1,6-dimethyl-7-oxo-pyrrolo[2,3-c]pyridin-4-yl)-2,6-dimethoxy-phenyl]methyl-methyl-amino]acetamide (62, 35 mg, 66.08 μmol) and 1-(2,6-dioxo-3-piperidyl)-6-oxo-pyridine-3-carboxylic acid (66, 16.53 mg, 66.08 μmol) in anhydrous DMF (4 mL) were added DIPEA (25.62 mg, 198.25 μmol, 34.53 uL) and PyBOP (51.58 mg, 99.13 μmol) under nitrogen atmosphere at room temperature. The resulting mixture was stirred at room temperature for 16 hours. The reaction mixture was concentrated under reduced pressure and purified by prep-HPLC SUNFIRE OBD C18(100×30)MM 5μ) Mobile phase: A: 0.1% TFA in water B: ACN to afford N-[2-[2-[2-[[2-[[4-(1,6-dimethyl-7-oxo-pyrrolo[2,3-c]pyridin-4-yl)-2,6-dimethoxy-phenyl]methyl-methyl-amino]acetyl]amino]ethoxy]ethoxy]ethyl]-1-(2,6-dioxo-3-piperidyl)-6-oxo-pyridine-3-carboxamide (Compound 219, 9 mg, 11.59 μmol, 17.54% yield) as a colorless liquid. ¹H NMR (400 MHz, Methanol-d₄) δ 8.23 (d, J=2.5 Hz, 1H), 7.89 (dd, J=9.5, 2.5 Hz, 1H), 7.38 (s, 1H), 7.28 (d, J=2.9 Hz, 1H), 6.93 (s, 2H), 6.54-6.48 (m, 2H), 4.49 (d, J=3.6 Hz, 2H), 4.18 (s, 3H), 3.97 (d, J=2.2 Hz, 6H), 3.93 (s, 1H), 3.86 (s, 1H), 3.67 (s, 3H), 3.63-3.57 (m, 7H), 3.51 (q, J=6.0, 5.5 Hz, 5H), 3.43-3.36 (m, 2H), 2.91 (s, 3H), 2.86-2.62 (m, 4H). LCMS (ES+): m/z 762 [M+H]⁺

### Degrader Synthesis 21: Compound 220

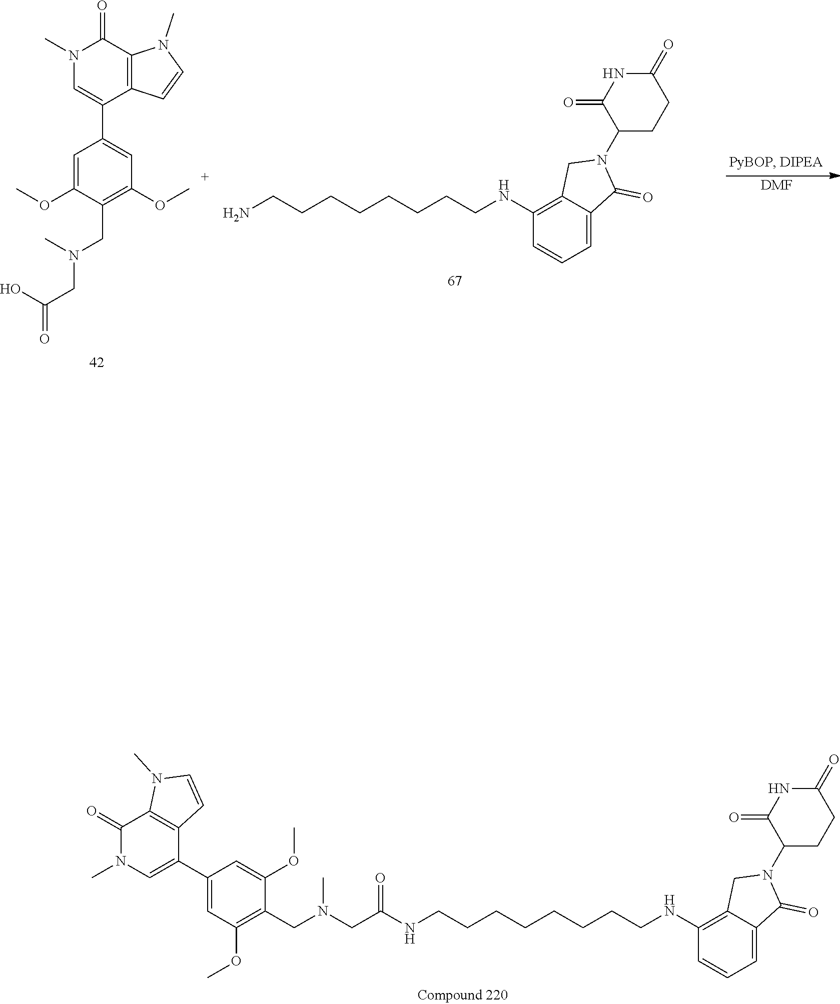

Into a 25 mL single-necked round-bottomed flask containing a well-stirred solution of 2-[[4-(1,6-dimethyl-7-oxo-pyrrolo[2,3-c]pyridin-4-yl)-2,6-dimethoxy-phenyl]methyl-methyl-amino]acetic acid (42, 50 mg, 125.18 μmol) and 3-[4-(8-aminooctylamino)-1-oxo-isoindolin-2-yl]piperidine-2,6-dione (67, 48.38 mg, 125.18 μmol) in DMF (5 mL) were added DIPEA (48.53 mg, 375.53 μmol, 65.41 uL) and PyBOP (78.17 mg, 150.21 μmol) under a nitrogen atmosphere at room temperature. The resulting mixture was stirred at room temperature for 16 hours. After completion, 20 mL of cold water was added and the reaction was stirred for 10 minutes. The resulting solid was filtered and dried to afford crude product that was purified by reverse phase prep purification (SUNFIRE OBD C18(100×30)MM 5μ) Mobile phase: A: 0.1% TFA in water B: ACN) to obtain 2-[[4-(1,6-dimethyl-7-oxo-pyrrolo[2,3-c]pyridin-4-yl)-2,6-dimethoxy-phenyl]methyl-methyl-amino]-N-[8-[[2-(2,6-dioxo-3-piperidyl)-1-oxo-isoindolin-4-yl]amino]octyl]acetamide (Compound 220, 5 mg, 6.51 μmol, 5.20% yield). LCMS (ES+): m/z 768 [M+H]+

### Degrader Synthesis 22: Compound 221

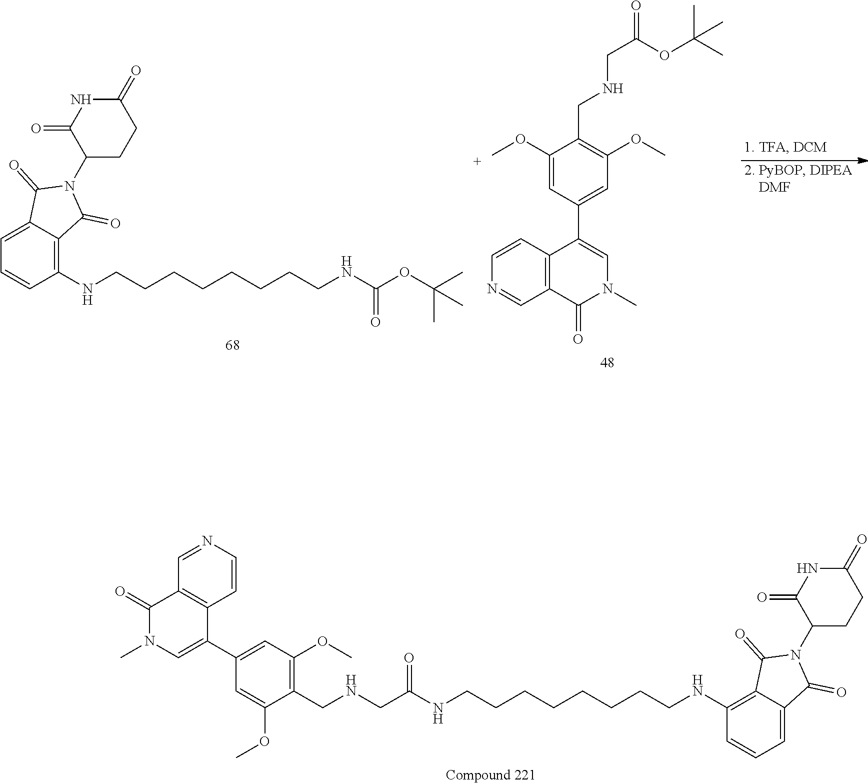

Into a 25 mL single-necked round-bottomed flask containing a well-stirred solution of tert-butyl N-[8-[[2-(2,6-dioxo-3-piperidyl)-1,3-dioxo-isoindolin-4-yl]amino]octyl]carbamate (68, 58.75 mg, 117.37 μmol) in anhydrous DCM (4 mL) was added TFA (8.92 mg, 78.25 μmol, 6.03 uL) under nitrogen atmosphere at room temperature. The resulting mixture was stirred at room temperature for 30 minutes and concentrated under reduced pressure. The resulting material was dissolved in DMF (3 mL) and 2-[[2,6-dimethoxy-4-(2-methyl-1-oxo-2,7-naphthyridin-4-yl)phenyl]methylamino]acetic acid (48, 30 mg, 78.25 μmol), DIPEA (10.11 mg, 78.25 μmol, 13.63 uL) and PyBOP (40.72 mg, 78.25 μmol) were added under a nitrogen atmosphere and the reaction stirred at room temperature for 16 hours. The reaction mixture was concentrated under reduced pressure and purified by prep-HPLC SUNFIRE OBD C18(100×30)MM 50 Mobile phase: A: 0.1% TFA in water B: ACN to afford 2-[[2,6-dimethoxy-4-(2-methyl-1-oxo-2,7-naphthyridin-4-yl)phenyl]methylamino]-N-[8-[[2-(2,6-dioxo-3-piperidyl)-1,3-dioxo-isoindolin-4-yl]amino]octyl]acetamide (Compound 221, 10 mg, 13.06 μmol, 16.69% yield) as a yellow solid. ¹H NMR (400 MHz, DMSO-d6) δ 11.08 (s, 1H), 9.48 (s, 1H), 8.72 (dd, J=6.2, 2.8 Hz, 1H), 8.62 (d, J=6.7 Hz, 1H), 8.00-7.94 (m, 1H), 7.69-7.63 (m, 1H), 7.60-7.54 (m, 1H), 7.08 (dd, J=8.7, 2.6 Hz, 1H), 7.01 (dd, J=6.9, 2.6 Hz, 1H), 6.76-6.70 (m, 2H), 6.52 (s, 1H), 5.05 (dd, J=12.3, 5.3 Hz, 1H), 4.11 (d, J=2.8 Hz, 3H), 3.77-3.73 (m, 6H), 3.63-3.58 (m, 3H), 3.28 (s, 2H), 3.17-3.07 (m, 2H), 2.93-2.80 (m, 2H), 2.62-2.55 (m, 1H), 2.07-1.97 (m, 1H), 1.62-1.52 (m, 2H), 1.51-1.41 (m, 2H), 1.30 (d, J=14.1 Hz, 8H). LCMS (ES+): m/z 766 [M+H]+

### Degrader Synthesis 23: Compound 222

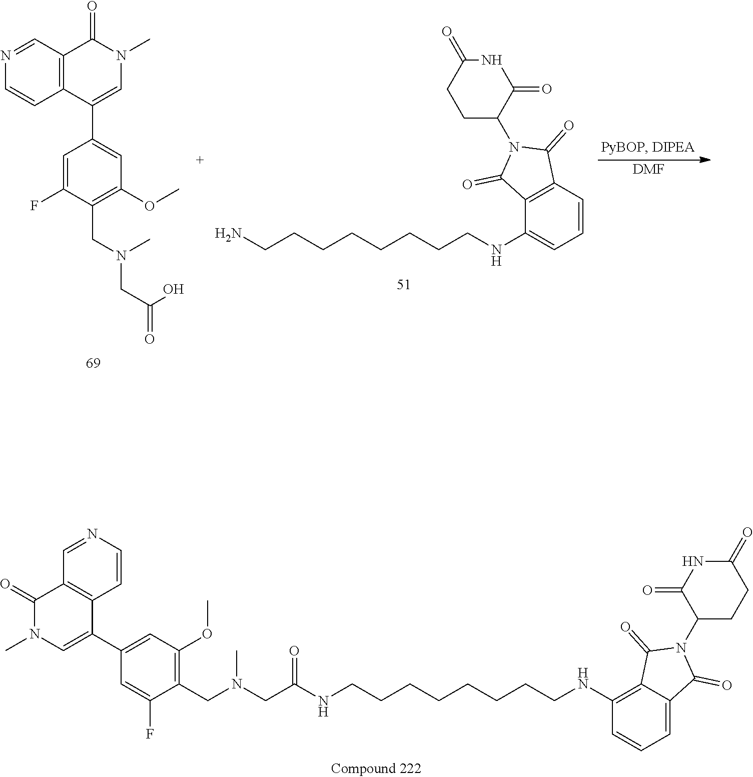

To a solution of 2-[[2-fluoro-6-methoxy-4-(2-methyl-1-oxo-2,7-naphthyridin-4-yl)phenyl]methyl-methyl-amino]acetic acid (69, 79.58 mg, 206.50 μmol) and 4-(8-aminooctylamino)-2-(2,6-dioxo-3-piperidyl)isoindoline-1,3-dione (51, 82.70 mg, 206.50 μmol) in DMF (10 mL) was added DIPEA (133.45 mg, 1.03 mmol, 179.85 uL) and PyBOP (161.19 mg, 309.75 μmol). The resulting mixture was stirred for 16 hours at 25° C. Water (20 mL) was added and the reaction mixture was extracted with ethyl acetate (3×25 mL). The combined organic extracts were dried over anhydrous sodium sulfate, filtered and excess solvent was evaporated under reduced pressure. The crude material was purified by reverse phase prep HPLC to yield N-[8-[[2-(2,6-dioxo-3-piperidyl)-1,3-dioxo-isoindolin-4-yl]amino]octyl]-2-[[2-fluoro-6-methoxy-4-(2-methyl-1-oxo-2,7-naphthyridin-4-yl)phenyl]methyl-methyl-amino]acetamide (Compound 222, 10 mg, 13.00 μmol, 6.30% yield). ¹H NMR (400 MHz, DMSO-d6) δ 11.09 (s, 1H), 9.43 (s, 1H), 8.72 (d, J=5.6 Hz, 1H), 7.91 (s, 1H), 7.61 (t, J=5.8 Hz, 1H), 7.58-7.52 (m, 2H), 7.05 (d, J=8.6 Hz, 1H), 7.02-6.93 (m, 3H), 6.50 (t, J=5.8 Hz, 1H), 5.04 (dd, J=12.8, 5.4 Hz, 1H), 3.87 (s, 3H), 3.65 (s, 2H), 3.58 (s, 3H), 3.25 (q, J=6.5 Hz, 2H), 3.11 (q, J=6.6 Hz, 2H), 2.98 (s, 2H), 2.93-2.81 (m, 1H), 2.63-2.54 (m, 2H), 2.20 (d, J=4.0 Hz, 3H), 2.07-1.96 (m, 1H), 1.57-1.48 (m, 2H), 1.45-1.37 (m, 2H), 1.34-1.22 (m, 8H). LCMS (ES+): m/z 768 [M+H]+

### Degrader Synthesis 24: Compound 223

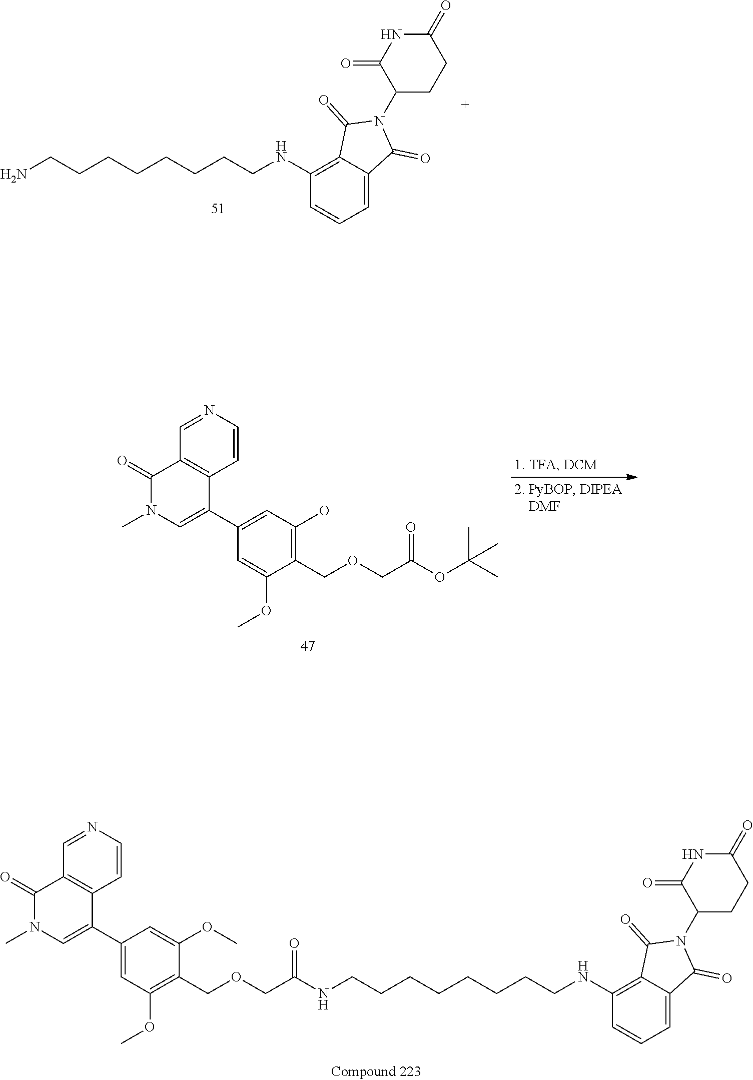 [SMILES: NCCCCCCCCNc1cccc2c1C(=O)N(C1CCC(=O)NC1=O)C2=O ; COc1cc(-c2cn(C)c(=O)c3cnccc23)cc(O)c1COCC(=O)OC(C)(C)C ; COc1cc(-c2cn(C)c(=O)c3cnccc23)cc(OC)c1COCC(=O)NCCCCCCCCNc1cccc2c1C(=O)N(C1CCC(=O)NC1=O)C2=O]

Into a 25 mL single-necked round-bottomed flask containing a well-stirred solution of tert-butyl 2-[[2,6-dimethoxy-4-(2-methyl-1-oxo-2,7-naphthyridin-4-yl)phenyl]methoxy]acetate (47, 100 mg, 227.02 μmol) in anhydrous DCM (4 mL) was added TFA (129.42 mg, 1.14 mmol, 87.45 uL) under nitrogen atmosphere at room temperature. The mixture was stirred at room temperature for 30 minutes and then concentrated under reduced pressure. The resulting material was dissolved in DMF (3 mL) and 4-(8-aminooctylamino)-2-(2,6-dioxo-3-piperidyl)isoindoline-1,3-dione (51, 100.01 mg, 249.72 μmol), DIPEA (88.02 mg, 681.06 μmol, 118.63 uL) and PyBOP (177.21 mg, 340.53 μmol) were added. The reaction was stirred under a nitrogen atmosphere at room temperature for 16 hours. The reaction mixture was concentrated under reduced pressure and purified by prep-HPLC SUNFIRE OBD C18(100×30)MM 5μ) Mobile phase: A: 0.1% TFA in water B: ACN to afford 2-[[2,6-dimethoxy-4-(2-methyl-1-oxo-2,7-naphthyridin-4-yl)phenyl]methoxy]-N-[8-[[2-(2,6-dioxo-3-piperidyl)-1,3-dioxo-isoindolin-4-yl]amino]octyl]acetamide (Compound 223, 6 mg, 7.33 μmol, 3.23% yield). 1H NMR (400 MHz, Methanol-d4) δ 9.55 (s, 1H), 8.66 (d, J=6.3 Hz, 1H), 7.99 (s, 1H), 7.87 (d, J=6.3 Hz, 1H), 7.57-7.46 (m, 1H), 7.01-6.96 (m, 2H), 6.77 (s, 2H), 5.03 (dd, J=12.2, 5.4 Hz, 1H), 4.75 (s, 2H), 3.96 (d, J=7.0 Hz, 2H), 3.89 (s, 6H), 3.72 (s, 3H), 3.26 (q, J=6.7 Hz, 3H), 2.91-2.79 (m, 1H), 2.77-2.63 (m, 3H), 2.14-2.04 (m, 1H), 1.71-1.58 (m, 2H), 1.55-1.49 (m, 2H), 1.45-1.31 (m, 10H). LCMS (ES+): m/z 767 [M+H]+

### Degrader Synthesis 25: Compound 224

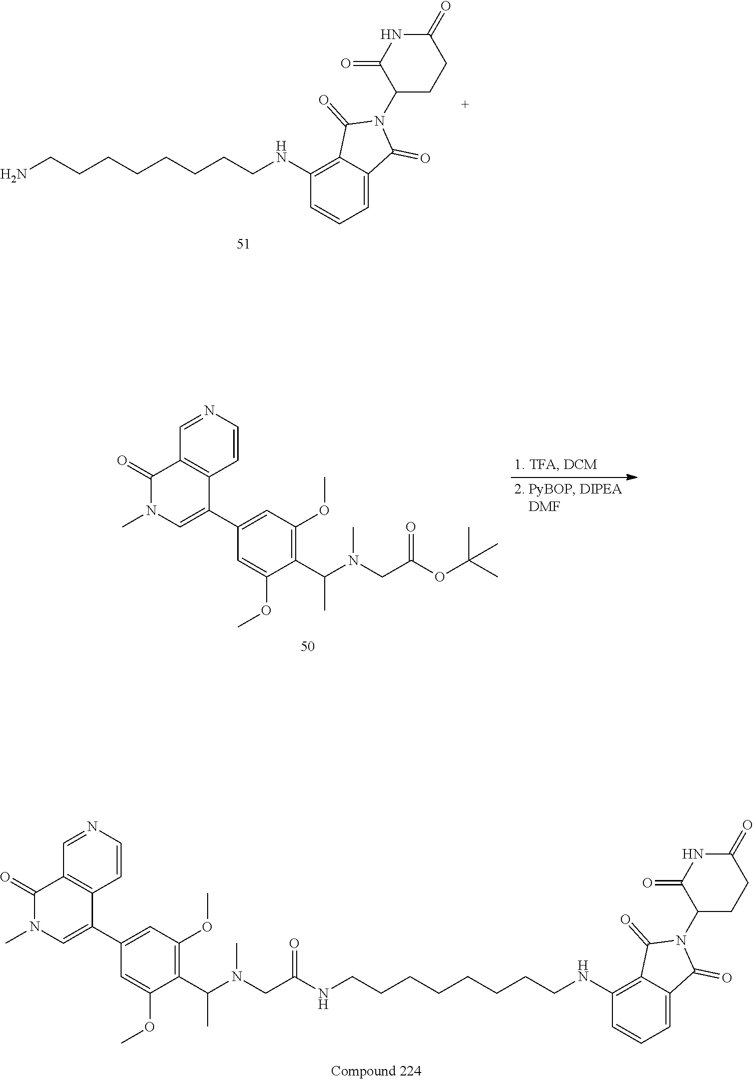 [SMILES: NCCCCCCCCNc1cccc2c1C(=O)N(C1CCC(=O)NC1=O)C2=O ; COc1cc(-c2cn(C)c(=O)c3cnccc23)cc(OC)c1C(C)N(C)CC(=O)OC(C)(C)C ; COc1cc(-c2cn(C)c(=O)c3cnccc23)cc(OC)c1C(C)N(C)CC(=O)NCCCCCCCCNc1cccc2c1C(=O)N(C1CCC(=O)NC1=O)C2=O]

Into a 25 mL single-necked round-bottomed flask containing a well-stirred solution of tert-butyl 2-[1-[2,6-dimethoxy-4-(2-methyl-1-oxo-2,7-naphthyridin-4-yl)phenyl]ethyl-methyl-amino]acetate (50, 45 mg, 96.25 μmol) in anhydrous DCM (4 mL) was added TFA (54.87 mg, 481.23 μmol, 37.07 uL) under nitrogen atmosphere at room temperature. The mixture was stirred at room temperature for 30 minutes and then concentrated under reduced pressure. The resulting material was dissolved in DMF (3 mL) and 4-(8-aminooctylamino)-2-(2,6-dioxo-3-piperidyl)isoindoline-1,3-dione (51, 38.54 mg, 96.25 μmol), DIPEA (37.32 mg, 288.74 μmol, 50.29 uL) and PyBOP (75.13 mg, 144.37 μmol) were added under nitrogen atmosphere at room temperature. The resulting mixture was stirred at room temperature for 16 hours. The reaction mixture was concentrated under reduced pressure and purified by prep-HPLC SUNFIRE OBD C18(100×30)MM 5μ) Mobile phase: A: 0.1% TFA in water B: ACN to afford 2-[1-[2,6-dimethoxy-4-(2-methyl-1-oxo-2,7-naphthyridin-4-yl)phenyl]ethyl-methyl-amino]-N-[8-[[2-(2,6-dioxo-3-piperidyl)-1,3-dioxo-isoindolin-4-yl]amino]octyl]acetamide (Compound 224, 12 mg, 13.30 μmol, 13.82% yield). LCMS (ES+): m/z 794 [M+H]+

### Example 2. Synthesis of MTH1 Degraders

### General Intermediate Scheme 1

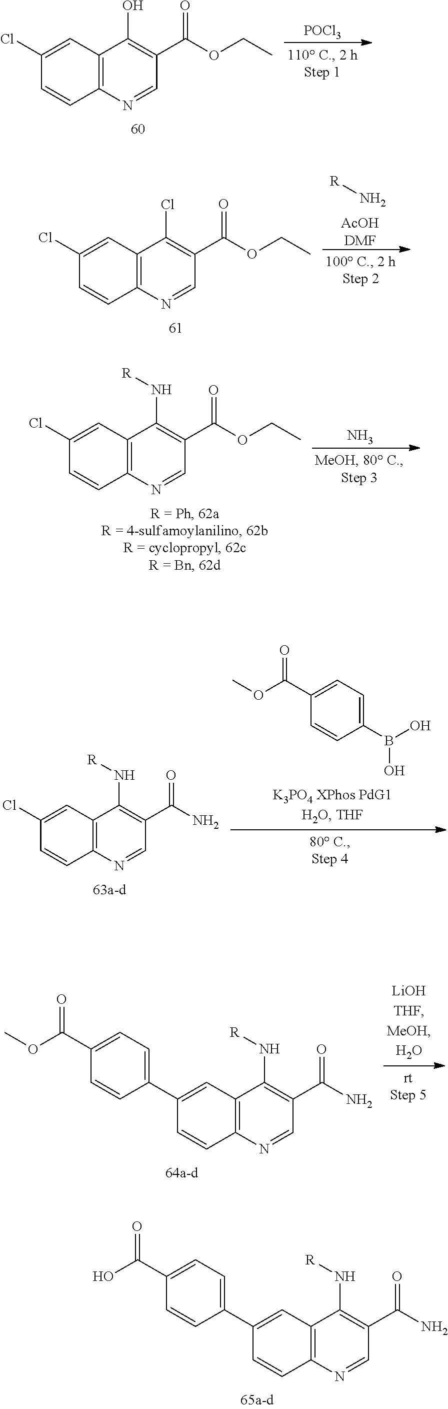

Step 1: An oven dried pressure tube was charged with a solution of ethyl 6-chloro-4-hydroxy-quinoline-3-carboxylate (60, 2 g, 7.95 mmol) in phosphorus oxychloride (1.22 g, 7.95 mmol, 15 mL) and the reaction mixture was heated to 110° C. for 2 hours. The reaction mixture was cooled to room temperature and concentrated under reduced pressure. The crude product was diluted with water (20 mL) and the product was extracted with ethyl acetate (2×100 mL). The combined organic layer was dried over anhydrous sodium sulfate, filtered and concentrated under reduced pressure to yield ethyl 4,6-dichloroquinoline-3-carboxylate (61, 1.9 g, 6.56 mmol, 82.52% yield) as yellow solid. LCMS (ES+): m/z 272 [M+H]+

Step 2 (Synthesis of Compound 62a): To a stirred solution of ethyl 4,6-dichloroquinoline-3-carboxylate (61, 1.0 g, 3.70 mmol) and aniline (413.72 mg, 4.44 mmol, 405.61 uL) in N,N-dimethyl formamide (15 mL) in a sealed tube was added acetic acid (222.32 mg, 3.70 mmol, 211.73 uL). The reaction mixture was sealed and heated to 100° C. for 2 hours. After completion, the reaction mixture was concentrated and the resulting solid was triturated with diethyl ether and filtered to yield pure product ethyl 4-anilino-6-chloro-quinoline-3-carboxylate (62a, 700 mg, 2.14 mmol, 57.86% yield) as an off-white colored solid. LCMS (ES+): m/z 327 [M+H]+

Step 2 (Synthesis of Compound 62b): To a stirred solution ethyl 4,6-dichloroquinoline-3-carboxylate (61, 1.0 g, 3.70 mmol) and 4-aminobenzenesulfonamide (765.04 mg, 4.44 mmol, 708.37 uL) in N,N-dimethyl formamide (20 mL) in a sealed tube was added acetic acid (222.32 mg, 3.70 mmol, 211.74 uL). The reaction mixture was sealed and heated to 100° C. for 2 hours. After completion, the reaction mixture concentrated and the resulting solid was triturated with diethyl ether and filtered to yield pure product ethyl 6-chloro-4-(4-sulfamoylanilino)quinoline-3-carboxylate (62b, 1.2 g, 2.87 mmol, 77.47% yield) as yellow colored solid. LCMS (ES+): m/z 406 [M+H]⁺

Step 2 (Synthesis of Compound 62c): To a stirred solution of ethyl 4,6-dichloroquinoline-3-carboxylate (61, 250 mg, 925.55 μmol) in DMF (5. mL) was added cyclopropyl amine (63.41 mg, 1.11 mmol, 76.96 uL) and DIPEA (358.86 mg, 2.78 mmol, 483.64 uL). The resulting solution was stirred for 2 hours at 100° C. The reaction was cooled to ambient temperature, diluted with water (25 mL) and extracted with ethyl acetate (2×20 mL). The combined organic extracts were washed with water and brine solution, dried over anhydrous sodium sulfate, and filtered. Excess solvent was removed under reduced pressure and the resulting crude material was purified by column chromatography on silica eluted with 5% methanol in dichloromethane to yield ethyl 6-chloro-4 (cyclopropylamino)quinoline-3-carboxylate (62c, 220 mg, 738.75 μmol, 79.82% yield) as an pale brown colored solid. LCMS (ES+): m/z 291 [M+H]+

Step 2 (Synthesis of Compound 62d): To a stirred solution of ethyl 4,6-dichloroquinoline-3-carboxylate (61, 250 mg, 925.55 μmol) in DMF (5.00 mL) was added phenylmethanamine (119.01 mg, 1.11 mmol) and DIPEA (358.86 mg, 2.78 mmol, 483.64 uL). The resulting mixture was stirred for 2 hours at 100° C. The reaction mixture was cooled to ambient temperature, diluted with water (25 mL) and extracted with ethyl acetate (2×20 mL). The combined organic extracts were washed with water and brine solution, dried over anhydrous sodium sulfate, and filtered. Excess solvent was removed under reduced pressure. The resulting crude material was purified by column chromatography on silica eluted with 5% methanol in dichloromethane to yield ethyl 4-(benzylamino)-6-chloro-quinoline-3-carboxylate (62d, 210 mg, 562.15 μmol, 60.74% yield) as a pale brown colored solid. LCMS (ES+): m/z 341 [M+H]+

Step 3: To a stirred solution of ethyl 4-anilino-6-chloro-quinoline-3-carboxylate (62a, 700 mg, 2.14 mmol) in methanol (5 mL) in a sealed tube was added ammonia (7 M, 15 mL) in methanol. The reaction mixture in sealed tube was heated to 80° C. for 16 hours. After completion. The reaction was concentrated and the resulting solid was triturated with diethyl ether and filtered to yield 4-anilino-6-chloro-quinoline-3-carboxamide (63a, 600 mg, 1.77 mmol, 82.79% yield) as light brown colored solid. LCMS (ES+): m/z 298 [M+H]+
Compounds 63b-d were also prepared using a similar procedure.

Alternative Step 3 (not shown): A round bottom flask was charged with ethyl 4-anilino-6-chloro-quinoline-3-carboxylate (62a, 1 g, 3.06 mmol) in THF (10 mL), methanol (8 mL) and water (10 mL). Lithium hydroxide powder (reagent grade (146.58 mg, 6.12 mmol)) was added and the reaction mixture was stirred for 16 hours at room temperature. The reaction mixture was concentrated under reduced pressure. The crude product was acidified with citric acid up to a pH of approximately 6. The solid was filtered and dried under vacuum to yield 4-anilino-6-chloro-quinoline-3-carboxylic acid (850 mg, 2.73 mmol, 89.08% yield) as a white solid. LCMS (ES+): m/z 299 [M+H]+

Step 4: XPhos-Pd-G1 (31.68 mg, 40.30 μmol) was added to a stirred solution of 4-anilino-6-chloro-quinoline-3-carboxamide (63a, 400 mg, 1.34 mmol), (4-methoxycarbonylphenyl)boronic acid (290.13 mg, 1.61 mmol) and potassium phosphate tribasic (712.93 mg, 3.36 mmol) in THF (20 mL) and water (5 mL). The reaction was stirred for 2 hours at 80° C. The reaction mixture was cooled to ambient temperature, diluted with water (15 mL) and extracted with ethyl acetate (2×20 mL). The combined organic extracts were dried over anhydrous sodium sulfate and filtered. Excess solvent was removed under reduced pressure. The resulting crude material was purified by column chromatography on silica eluted with 5% methanol in dichloromethane to yield methyl 4-(4-anilino-3-carbamoyl-6-quinolyl)benzoate (64a, 370 mg, 915.17 μmol, 68.12% yield, 98.30% purity) as an off white solid. LCMS (ES+): m/z 398 [M+H]+
Compounds 64b-d were also prepared using a similar procedure.

Alternative Step 4 (not shown): An oven-dried round bottom flask was charged with a solution of 4-anilino-6-chloro-quinoline-3-carboxylic acid (850 mg, 2.85 mmol) in DMF (10 mL). Ammonium chloride (761.04 mg, 14.23 mmol, 497.41 uL), DIPEA (1.84 g, 14.23 mmol, 2.48 mL) and HATU (1.30 g, 3.41 mmol) were added. The reaction mixture was stirred for 16 hours at room temperature. The reaction mixture was quenched with water (20 mL) and the product was filtered. The solid was dried under vacuum to yield 4-anilino-6-chloro-quinoline-3-carboxamide (63a, 530 mg, 1.58 mmol, 55.58% yield) as a white solid. LCMS (ES+): m/z 298 [M+H]⁺

Step 5: A round bottom flask was charged with methyl 4-(4-anilino-3-carbamoyl-6-quinolyl)benzoate (64a, 390 mg, 981.32 μmol) in THF (10 mL), methanol (5 mL) and water (10 mL). Lithium hydroxide powder (reagent grade (47.01 mg, 1.96 mmol)) was added and the reaction mixture was stirred for 16 hours at room temperature. The reaction mixture was concentrated under reduced pressure. The crude product was acidified with citric acid up to a pH of approximately 6. The solid was filtered and dried over reduced pressure to yield 4-(4-anilino-3-carbamoyl-6-quinolyl)benzoic acid (65a, 360 mg, 748.83 μmol, 76.31% yield) as white solid. LCMS (ES+): m/z 384 [M+H]+
Compounds 65b-d were also prepared using a similar procedure.

### General Intermediate Scheme 2

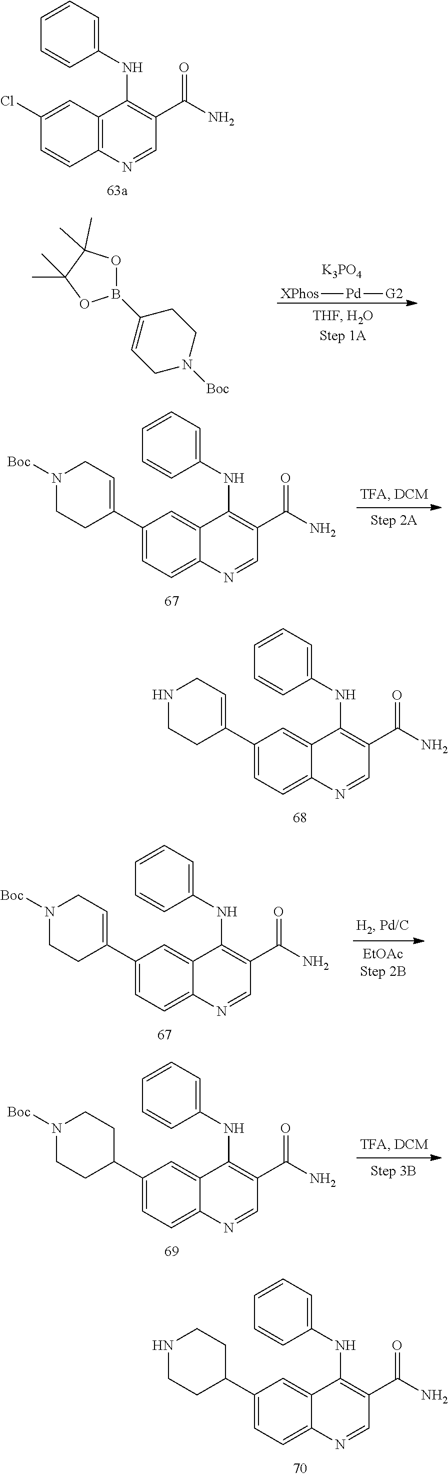

Step 1A: XPhos-Pd-G2 (23.78 mg, 30.23 μmol) was added to a stirred solution of 4-anilino-6-chloro-quinoline-3-carboxamide (63a, 300 mg, 1.01 mmol), tert-butyl 4-(4,4,5,5-tetramethyl-1,3,2-dioxaborolan-2-yl)-3,6-dihydro-2H-pyridine-1-carboxylate (373.87 mg, 1.21 mmol) and potassium phosphate tribasic (534.70 mg, 2.52 mmol) in THF (10.0 mL) and water (2.0 mL). The reaction was stirred for 2 hours at 80° C. The resulting mixture was cooled to ambient temperature, diluted with water (15 mL) and extracted with ethyl acetate (2×20 mL). The combined organic extracts were dried over anhydrous sodium sulfate and filtered. Excess solvent was evaporated under reduced pressure. The resulting crude was purified by column chromatography on silica eluted with 5% methanol in dichloromethane to yield tert-butyl 4-(4-anilino-3-carbamoyl-6-quinolyl)-3,6-dihydro-2H-pyridine-1-carboxylate (67, 400 mg, 840.09 μmol, 83.38% yield) as an off white solid. LCMS (ES+): m/z 445 [M+H]+

Step 2A: To a stirred solution of tert-butyl 4-(4-anilino-3-carbamoyl-6-quinolyl)-3,6-dihydro-2H-pyridine-1-carboxylate (67, 100 mg, 224.96 μmol) in DCM (5 mL) was added trifluoroacetic acid (1.48 g, 12.98 mmol, 1 mL) and the reaction was stirred for 1 hour at 25° C. The resulting mixture was concentrated under reduced pressure to yield 4-anilino-6-(1,2,3,6-tetrahydropyridin-4-yl)quinoline-3-carboxamide (68, 75 mg, 212.89 μmol, 94.63% yield) as an yellow solid. LCMS (ES+): m/z 345 [M+H]+

Step 2B: To a stirred solution of tert-butyl 4-(4-anilino-3-carbamoyl-6-quinolyl)-3,6-dihydro-2H-pyridine-1-carboxylate (67, 400 mg, 899.84 μmol) in ethyl acetate (20 mL) was added palladium, 10% on carbon (100 mg, 899.84 μmol) and the reaction was stirred for 2 hours at 25° C. under 10 atm H₂ pressure (ballon). The resulting solution was filtered through a celite cake and washed with ethyl acetate. The excess solvent was evaporated under reduced pressure to yield tert-butyl 4-(4-anilino-3-carbamoyl-6-quinolyl)piperidine-1-carboxylate (69, 360 mg, 717.52 μmol, 79.74% yield) as an off white solid. LCMS (ES+): m/z 447 [M+H]+

Step 3B: To a stirred solution of tert-butyl 4-(4-anilino-3-carbamoyl-6-quinolyl)piperidine-1-carboxylate (69, 360 mg, 806.20 μmol) in dichloromethane (5.0 mL) was added trifluoroacetic acid (459.63 mg, 4.03 mmol, 310.56 uL) and the reaction was stirred for 1 hour at 25° C. The resulting mixture was concentrated under reduced pressure to yield 4-anilino-6-(4-piperidyl)quinoline-3-carboxamide (70, 260 mg, 731.54 μmol, 90.74% yield) as a yellow solid. LCMS (ES+): m/z 347 [M+H]⁺

### General Intermediate Scheme 3

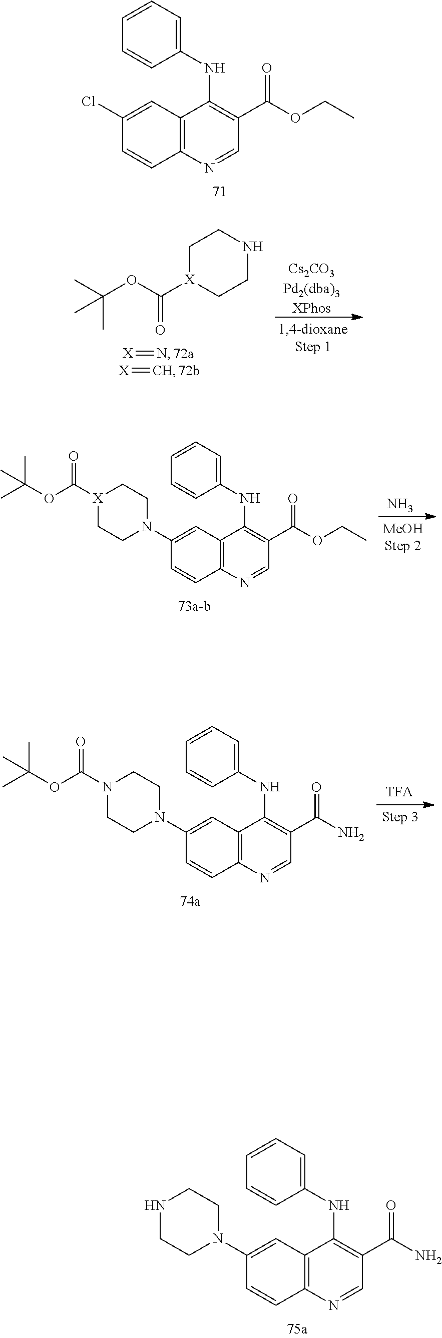

Step 1 (Synthesis of Compound 73a): A stirred solution of ethyl 4-anilino-6-chloro-quinoline-3-carboxylate (71, 1.0 g, 3.06 mmol), tert-butyl piperazine-1-carboxylate (72a, 683.96 mg, 3.67 mmol) and cesium carbonate (2.49 g, 7.65 mmol) in 1,4 dioxane (20.0 mL) was purged with nitrogen for 5 minutes. 2-Dicyclohexylphosphino-2′,4′,6′-triisopropylbiphenyl (291.77 mg, 612.04 umol) and Pd₂(dba)₃ (28.02 mg, 30.60 umol) was added and the reaction was stirred for 16 hours at 100° C. The reaction mixture was cooled to ambient temperature, filtered through a celite cake, and washed with ethyl acetate. The combined filtrate was dried over anhydrous sodium sulphate, filtered and the excess solvent was evaporated under reduced pressure. The resulting crude material was purified by column chromatography on silica eluted with 5% methanol in dichloromethane to yield ethyl 4-anilino-6-(4-tert-butoxycarbonylpiperazin-1-yl)quinoline-3-carboxylate (73a, 1.2 g, 2.39 mmol, 77.97% yield) as an off white solid. LCMS (ES+): m/z 477 [M+H]+

Step 1 (Synthesis of Compound 73b): Into a 50 mL sealed tube containing a mixture of ethyl 4-anilino-6-chloro-quinoline-3-carboxylate (71, 300 mg, 918.06 umol) and tert-butyl piperidine-4-carboxylate hydrochloride (72b, 244.27 mg, 1.10 mmol) in anhydrous 1,4 dioxane (10 mL) was added cesium carbonate (747.81 mg, 2.30 mmol). Argon gas was bubbled through the reaction mixture for 10 minutes. X-phos (87.53 mg, 183.61 umol) and tris(dibenzylideneacetone)dipalladium(0) (84.07 mg, 91.81 umol) were added and again the resulting suspension was purged with argon gas for 10 minutes. The contents were stirred at 100° C. under closed condition. The reaction was monitored by TLC and found to be complete by 16 hours. The reaction mixture was passed through a pad of Celite and the filtrate was concentrated under reduced pressure to afford the crude mass. The crude material was purified by silica-gel (230-400 mesh) with 7:3 EtOAc/petroleum ether to generate ethyl 4-anilino-6-(4-tert-butoxycarbonyl-1-piperidyl)quinoline-3-carboxylate (73b, 0.3 g, 630.81 umol, 68.71% yield) as a brown solid. LCMS [476.2 (M+H)+].

Step 2: A stirred solution of ethyl 4-anilino-6-(4-tert-butoxycarbonylpiperazin-1-yl)quinoline-3-carboxylate (73a, 1.6 g, 3.36 mmol) in ammonia in methanol (3.36 mmol, 15 mL) was stirred for 24 hours at 80° C. The reaction mixture was cooled to ambient temperature and excess solvent was evaporated under reduced pressure. The resulting crude material was purified by column chromatography on silica eluted with 10% methanol in dichloromethane to yield tert-butyl 4-(4-anilino-3-carbamoyl-6-quinolyl)piperazine-1-carboxylate (74a, 1.3 g, 2.88 mmol, 85.86% yield) as an yellow solid. LCMS (ES+): m/z 448 [M+H]+

Step 3: tert-butyl 4-(4-anilino-3-carbamoyl-6-quinolyl)piperazine-1-carboxylate (74a) was deprotected with TFA in DCM at room temperature as described in Step 3B in General Intermediate Scheme 2. Upon completion of the reaction, the solvent was removed under reduced pressure to give 4-(phenylamino)-6-(piperazin-1-yl)quinoline-3-carboxamide (75b). The resulting crude material was taken on to the next step without further purification.

### General Intermediate Scheme 4

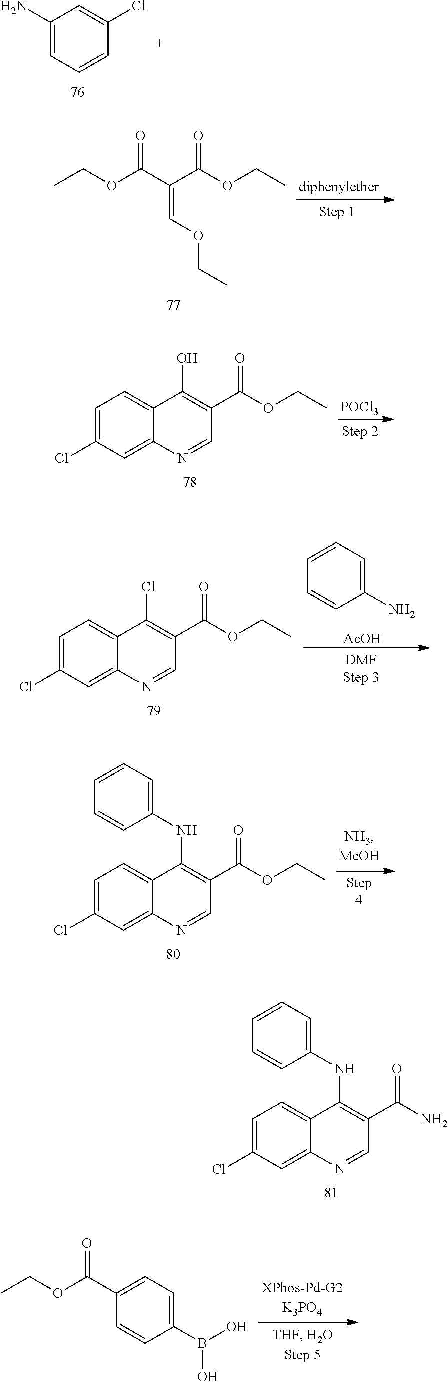

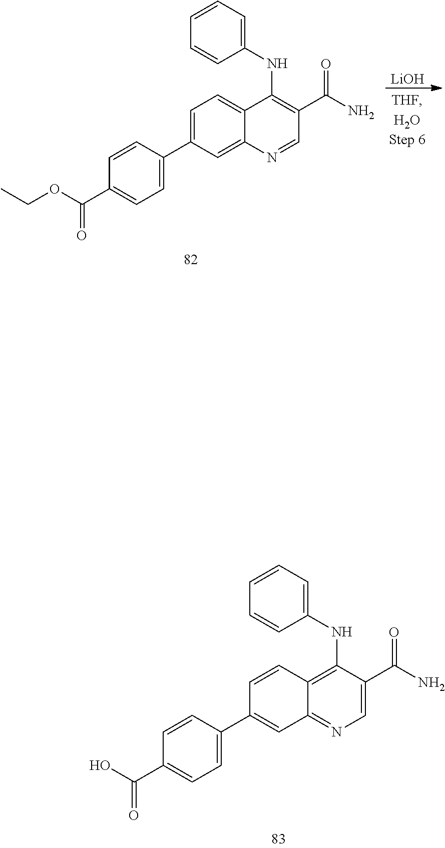

Step 1: A mixture of 3-chloroaniline (76, 1 g, 7.83873 mmol) and diethyl 2-(ethoxymethylene)malonate (77, 2.54244 g, 11.7580 mmol) was heated at 100° C. for 2 hours and at 165° C. for 1 hour. The resulting reaction mixture was dissolved in diphenylether (14 ml) and the solution was heated at reflux for 5 hours. The reaction mixture was cooled to room temperature and diluted with pet ether. The resulting solid was filtered, washed with pet ether followed by diethtyl ether, and dried to yield ethyl 7-chloro-4-hydroxyquinoline-3-carboxylate (78, 699.9 mg, 2.78 mmol, 35.4% yield) as an off white solid. LCMS (ES+): m/z 252 [M+H]+

Step 2: A stirred solution of ethyl 7-chloro-4-hydroxyquinoline-3-carboxylate (78, 10.0 g, 39.7 mmol) in phosphorus(V) oxychloride (80.0 mL, 854 mmol) was heated to 110° C. and stirred for 3 hours at 110° C. The reaction was cooled to room temperature and excess solvent was evaporated under reduced pressure. The resulting solid was dissolved in ethyl acetate, washed with water and brine solution, dried over anhydrous sodium sulfate, and filtered. Excess solvent was evaporated under reduced pressure to yield ethyl 4,7-dichloroquinoline-3-carboxylate (79, 8.0 g, 29.6 mmol, 75.56%) as a pale yellow colored solid. LCMS (ES+): m/z 272 [M+H]+

Step 3: An oven-dried pressure tube was charged with a solution of ethyl 4,7-dichloroquinoline-3-carboxylate (79, 100 mg, 370 μmol) in dimethylformamide (1 mL). Aniline (41.2 mg, 443 μmol) and acetic acid (11.1 mg, 185 μmol) were added at room temperature. The reaction mixture was heated to 100° C. for one hour and the reaction mixture was cooled room temperature. The reaction mixture was diluted with water (3 mL) and the product was extracted with ethyl acetate (2×10 mL). The organic layer was dried over anhydrous sodium sulfate, filtered and concentrated under reduced pressure. The crude mixture was purified by column chromatography on silica (9% ethyl acetate\pet ether) to yield ethyl 7-chloro-4-(phenylamino)quinoline-3-carboxylate as yellow solid (80, 101 mg, 310 mmol, 84.1% yield) LCMS (ES+): m/z 327 [M+H]⁺

Step 4: A stirred solution of ethyl 7-chloro-4-(phenylamino)quinoline-3-carboxylate (80, 1.0 g, 3.06 mmol) in ethanol (10.0 mL) was purged with ammonia gas for 10 minutes at −30° C. The resulting solution was heated to 80° C. for 12 hours. The reaction was cooled to room temperature and the excess solvent was removed under reduced pressure. The resulting crude material was purified by column chromatography on silica eluted with 5% methanol in dichloromethane to yield 7-chloro-4-(phenylamino)quinoline-3-carboxamide (81, 434 mg, 1.45 mmol, 47.6%) as a yellow colored solid. LCMS (ES+): m/z 298 [M+H]+

Step 5: To a stirred solution of 7-chloro-4-(phenylamino)quinoline-3-carboxamide (81, 20.0 mg, 0.06717 mmol) in THF (2.0 mL) and water (0.5 mL) was added (4-(ethoxycarbonyl)phenyl)boronic acid (16.9 mg, 0.08732 mmol) and potassium phosphate tribasic (0.1477 mmol). The resulting reaction mixture was purged with nitrogen for 5 minutes and XPhos-Pd-G2 (1.58 mg, 0.002015 mmol) was added. The reaction mixture was heated to 75° C. and stirred for 5 hours at 75° C. The reaction was then cooled to room temperature, diluted with ethyl acetate, filtered through a cellite bed, and washed with ethyl acetate. The resulting solution was washed with water and brine solution, dried over anhydrous sodium sulfate, filtered and concentrated under reduced pressure to yield ethyl 4-(3-carbamoyl-4-(phenylamino)quinolin-7-yl)benzoate (82, 33.4 mg, 0.0818 mmol, 131% yield) as pale brown colored oil. LCMS (ES+): m/z 412 [M+H]+

Step 6: To a stirred solution of ethyl 4-(3-carbamoyl-4-(phenylamino)quinolin-7-yl)benzoate (82, 1.1 g, 2.67 mmol) in THF (15 mL) and water (15 mL) was added lithium hydroxide monohydrate (13.3 mmol). The reaction was stirred overnight at room temperature. The reaction mixture was concentrated under reduced pressure and extracted with diethyl ether (2×25 mL). Saturated citric acid solution was added to the aqueous layer until the pH reached approximately 4. The resulting solid compound was filtered and washed with water and acetone to yield 4-(3-carbamoyl-4-(phenylamino)quinolin-7-yl)benzoic acid (83, 621 mg, 1.62 mmol, 60.8% yield) as a colorless solid. LCMS (ES+): m/z 384 [M+H]+

### General Intermediate Scheme 5

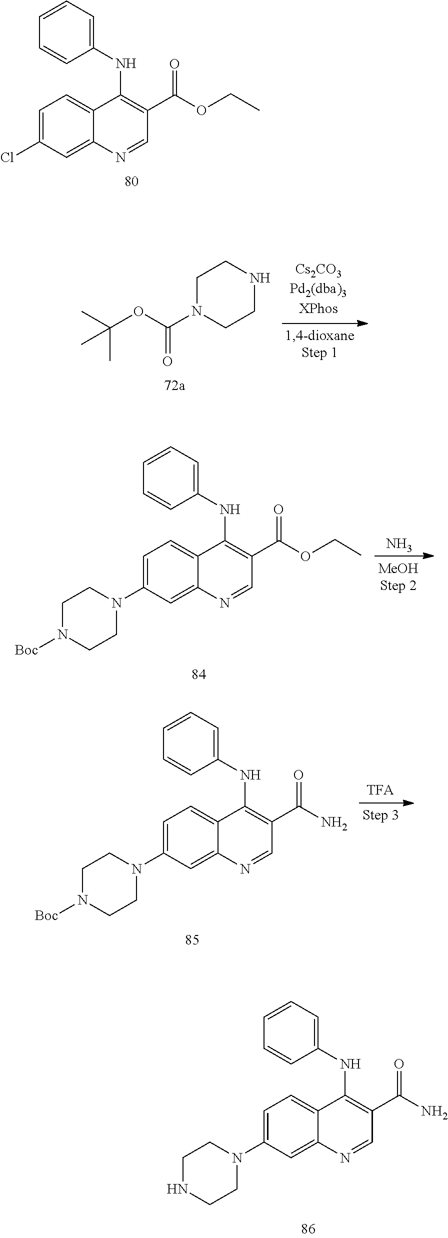

Step 1: To a stirred solution of ethyl 7-chloro-4-(phenylamino)quinoline-3-carboxylate (80, 1.0 g, 3.06 mmol) in 1,4 dioxane (20 mL) was added tert-butyl piperazine-1-carboxylate (72a, 854 mg, 4.59 mmol) and cesium carbonate (4.59 mmol). The resulting reaction mixture was purged with nitrogen for 5 minutes and XPhos (459 μmol) and tris(dibenzylideneacetone)dipalladium(0) (280 mg, 306 μmol) were added. The reaction mixture was heated to 110° C. and stirred for 10 hours at 100° C. The reaction was then cooled to room temperature, diluted with ethyl acetate, filtered through cellite bed, and washed with ethyl acetate. The resulting solution was washed with water and brine solution, dried over anhydrous sodium sulphate, filtered and concentrated under reduced pressure to yield brown colored oil. The resulting crude material was purified by column chromatography on silica eluted with 5% methanol in dichloromethane to yield ethyl 7-(4-(tert-butoxycarbonyl)piperazin-1-yl)-4-(phenylamino)quinoline-3-carboxylate (84, 969 mg, 2.03 mmol, 66.8%) as a yellow colored solid. LCMS (ES+): m/z 477 [M+H]+

Step 2: A stirred solution of ethyl 7-(4-(tert-butoxycarbonyl)piperazin-1-yl)-4-(phenylamino)quinoline-3-carboxylate (84, 30 mg, 0.06295 mmol) in ammonia in methanol (595 mg, 35.0 mmol) was heated to 80° C. and stirred overnight at 80° C. The reaction was then cooled to room temperature and concentrated under reduced pressure. The resulting crude material was purified by column chromatography on silica eluted with 5% methanol in dichloromethane to yield tert-butyl 4-(3-carbamoyl-4-(phenylamino)quinolin-7-yl)piperazine-1-carboxylate (85, 30.0 mg, 0.06703 mmol, 106%) as a yellow colored solid. LCMS (ES+): m/z 448 [M+H]

Step 3: tert-butyl 4-(3-carbamoyl-4-(phenylamino)quinolin-7-yl)piperazine-1-carboxylate (85) was deprotected with TFA in DCM at room temperature as described in Step 3B in General Intermediate Scheme 2. Upon completion of the reaction, the solvent was removed under reduced pressure to give 4-(phenylamino)-7-(piperazin-1-yl)quinoline-3-carboxamide (86). The resulting crude material was taken on to the next step without further purification.

### General Intermediate Scheme 6

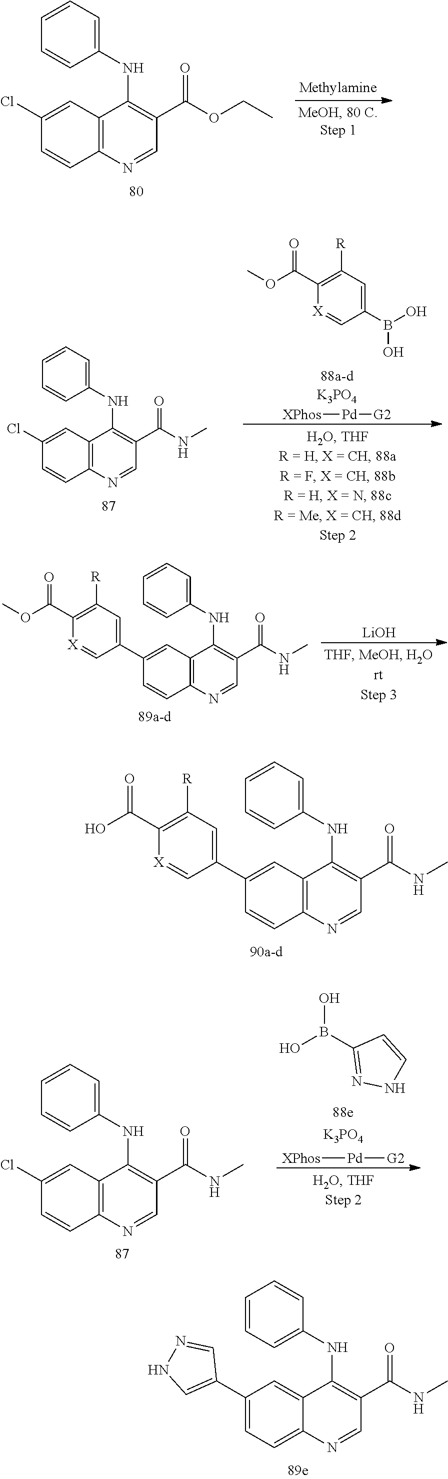

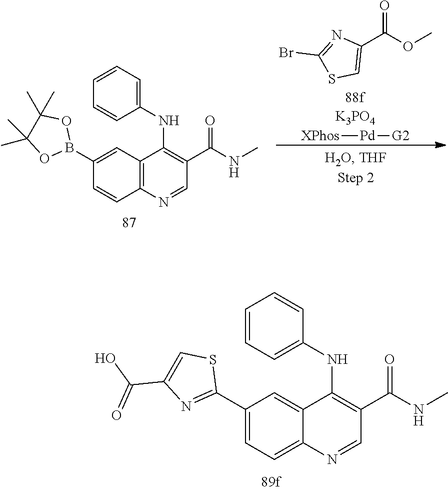

Step 1: To a stirred solution of ethyl 4-anilino-6-chloro-quinoline-3-carboxylate (80, 3.0 g, 9.18 mmol) was added methylamine (30% in methanol (26.97 g, 868.41 mmol, 30 mL)) and the reaction was stirred for 3 hours at 80° C. The reaction mixture was cooled to ambient temperature and concentrated under reduced pressure. The resulting crude material was purified by column chromatography on silica eluted with 10% methanol in dichloromethane to yield 4-anilino-6-chloro-N-methyl-quinoline-3-carboxamide (87, 2.2 g, 6.96 mmol, 75.87% yield) as a yellow solid. LCMS (ES+): m/z 312 [M+H]+

Step 2 (Synthesis of Compound 89a): To a stirred solution of 4-anilino-6-chloro-N-methyl-quinoline-3-carboxamide (87, 150 mg, 481.13 μmol), (4-methoxycarbonylphenyl)boronic acid (88a, 112.56 mg, 625.47 μmol) and potassium phosphate tribasic (255.32 mg, 1.20 mmol) in THF (5 mL) and water (1 mL), was added XPhos-Pd-G2 (11.35 mg, 14.43 μmol) and the reaction was stirred for 2 hours at 80° C. The reaction mixture was cooled to ambient temperature, diluted with water (15 mL), and extracted with ethyl acetate (2×20 mL). The combined organic extracts were dried over anhydrous sodium sulfate, filtered and the excess solvent was evaporated under reduced pressure. The resulting crude material was purified by column chromatography on silica eluted with 5% methanol in dichloromethane to yield methyl 4-[4-anilino-3-(methylcarbamoyl)-6-quinolyl]benzoate (89a, 180 mg, 412.06 μmol, 85.64% yield) as an off white solid. LCMS (ES+): m/z 412 [M+H]⁺

Step 2 (Synthesis of Compound 89b): Into a 20 mL sealed tube containing a mixture of 4-anilino-6-chloro-N-methyl-quinoline-3-carboxamide (87, 0.15 g, 481.13 μmol) and (3-fluoro-4-methoxycarbonyl-phenyl)boronic acid (88b, 142.86 mg, 721.70 μmol) in THF (8 mL) and water (2 mL) was added potassium phosphate tribasic anhydrous (255.32 mg, 1.20 mmol). Argon gas was bubbled through the reaction mixture for 5 minutes before XPhos-Pd-G2 (11.36 mg, 14.43 μmol) was added and the resulting suspension was purged with argon gas for an additional 5 minutes. The contents were heated at 80° C. for 2 hours. After completion, the reaction was filtered through a celite bed and the obtained filterate was extracted with EtOAc twice. The combined organic layers were concentrated to afford crude methyl 4-[4-anilino-3-(methylcarbamoyl)-6-quinolyl]-2-fluoro-benzoate (89b, 0.2 g, 465.72 mol, 96.80% yield). LCMS (ES+): m/z 430 [M+H]⁺

Step 2 (Synthesis of Compound 89c): An oven-dried pressure tube was charged with a solution of 4-anilino-6-chloro-N-methyl-quinoline-3-carboxamide (87, 456.08 mg, 1.46 mmol) in dioxane (10 mL) and cesium carbonate (1.19 g, 3.66 mmol) and (6-methoxycarbonyl-3-pyridyl)boronic acid (88c, 317.66 mg, 1.76 mmol) were added. The reaction mixture was purged with nitrogen for 5 minutes and XPhos (224.95 mg, 292.58 mol) and Pd₂(dba)₃ (133.96 mg, 146.29 mol) were added. The reaction mixture was heated to 100° C. for 2 hours and cooled to room temperature. The reaction mixture was diluted with water (15 mL) and the product was extracted with ethyl acetate (2×80 mL). The combined organic layers were dried over anhydrous sodium sulfate, filtered and concentrated under reduced pressure. The crude mixture was purified by column chromatography on silica (4% methanol\dichloromethane) to yield methyl 5-[4-anilino-3-(methylcarbamoyl)-6-quinolyl]pyridine-2-carboxylate (89c, 510 mg, 1.20 mmol, 81.76% yield) as yellow solid. LCMS (ES+): m/z 413 [M+H]⁺

Step 2 (Synthesis of Compound 89d): Into a 20 mL sealed tube containing a mixture of 4-anilino-6-chloro-N-methyl-quinoline-3-carboxamide (87, 0.15 g, 481.13 μmol) and (4-methoxycarbonyl-3-methyl-phenyl)boronic acid (88d, 140.00 mg, 721.70 μmol) in THF (8 mL) and water (2 mL) was added potassium phosphate tribasic anhydrous (255.32 mg, 1.20 mmol). Argon gas was bubbled through the reaction mixture for 5 minutes before XPhos-Pd-G2 (11.36 mg, 14.43 μmol). The resulting suspension was purged with argon gas for an additional 5 minutes. The contents were heated at 80° C. for 2 hours. After completion, the reaction was filtered through a celite bed and the obtained filterate was extracted with EtOAc twice. The combined organic layers were concentrated to afford crude methyl 4-[4-anilino-3-(methylcarbamoyl)-6-quinolyl]-2-methyl-benzoate (89d, 200 mg, 366.65 μmol, 76.20% yield). LCMS (ES+): m/z 426 [M+H]⁺

Step 2 (Synthesis of Compound 89e): To a stirred solution of 4-anilino-6-chloro-N-methyl-quinoline-3-carboxamide (87, 200 mg, 641.51 μmol), 1H-pyrazol-3-ylboronic acid (88e, 78.96 mg, 705.66 μmol) and potassium phosphate tribasic (340.43 mg, 1.60 mmol) in THF (5 mL) and water (2 mL) was added X-Phos-Pd-G2 (15.14 mg, 19.25 μmol). The reaction was stirred for 24 hours at 80° C. The resulting mixture was cooled to ambient temperature, diluted with water (15 mL) and extracted with ethyl acetate (2×20 mL). The combined organic extracts were dried over anhydrous sodium sulfate, filtered and concentrated under reduced pressure. The resulting crude material was purified by column chromatography on silica eluted with 5% methanol in dichloromethane to yield 4-anilino-N-methyl-6-(1H-pyrazol-4-yl)quinoline-3-carboxamide (89e, 170 mg, 460.97 μmol, 71.86% yield) as an off white solid. LCMS (ES+): m/z 344 [M+H]+

Step 2 (Synthesis of Compound 89f): To a solution of 4-anilino-N-methyl-6-(4,4,5,5-tetramethyl-1,3,2-dioxaborolan-2-yl)quinoline-3-carboxamide (87, 500 mg, 1.24 mmol) and methyl 2-bromothiazole-4-carboxylate (88f, 412.97 mg, 1.86 mmol) in water (5 mL) and THF (10 mL) was added potassium phosphate tribasic anhydrous (789.54 mg, 3.72 mmol). The reaction mixture was degasified for 2 minutes followed by the addition of 2^{nd} generation XPhos Precatalyst (97.48 mg, 123.98 μmol) under nitrogen atmosphere at room temperature. The resulting mixture was heated at 80° C. for 4 hours. The reaction mixture was diluted with water and extracted with ethyl acetate. The combined organics were dried over anhydrous sodium sulfate and concentrated to yield crude product that was purified by column chromatography using 5% methanol in dichloromethane as eluent to yield methyl 2-[4-anilino-3-(methylcarbamoyl)-6-quinolyl]thiazole-4-carboxylate (89f, 350 mg, 501.83 μmol, 40.48% yield). LCMS (ES+): m/z 419 [M+H]+

Step 3 (Synthesis of Compound 90a): A round bottom flask was charged with methyl 4-[4-anilino-3-(methylcarbamoyl)-6-quinolyl]benzoate (89a, 100 mg, 243.04 μmol) in THF (2 mL), methanol (0.5 mL) and water (2 mL). Lithium hydroxide powder (reagent grade (11.64 mg, 486.08 μmol)) was added and the reaction mixture was stirred for 16 hours at room temperature. The reaction mixture was concentrated under reduced pressure. The crude product was acidified with citric acid up to a pH of approximately 6. The solid was filtered and dried under reduced pressure to yield 4-[4-anilino-3-(methylcarbamoyl)-6-quinolyl]benzoic acid (90a, 90 mg, 220.28 μmol, 90.63% yield) as a white solid. LCMS (ES+): m/z 398 [M+H]+

Step 3 (Synthesis of Compound 90b): Into a 25 mL single-necked round-bottomed flask containing a well-stirred solution of methyl 4-[4-anilino-3-(methylcarbamoyl)-6-quinolyl]-2-fluoro-benzoate (89b, 280 mg, 652.01 μmol) in THF (4 mL) was added lithium hydroxide powder (reagent grade (78.08 mg, 3.26 mmol)) in water (4 mL) under nitrogen atmosphere. The resulting mixture was stirred at room temperature for 16 hours. The reaction mixture was concentrated under reduced pressure and the aqueous layer was acidified with saturated citric acid solution until pH=3. The resulting solid was filtered and dried to afford 4-[4-anilino-3-(methylcarbamoyl)-6-quinolyl]-2-fluoro-benzoic acid (90b, 250 mg, 601.81 μmol, 92.30% yield) as a brown solid. LCMS [416.2 (M+H)⁺].

Step 3 (Synthesis of Compound 90c): A round bottom flask was charged with methyl 5-[4-anilino-3-(methylcarbamoyl)-6-quinolyl]pyridine-2-carboxylate (89c, 500 mg, 1.21 mmol) in THF (10 mL), methanol (5 mL) and water (10 mL). Lithium hydroxide powder (reagent grade (58.07 mg, 2.42 mmol)) was added and the reaction mixture was stirred for 16 hours at room temperature. The reaction mixture was concentrated under reduced pressure. The crude product was acidified with citric acid to a pH of approximately 6. The solid was filtered and dried under vacuum to yield 5-[4-anilino-3-(methylcarbamoyl)-6-quinolyl]pyridine-2-carboxylic acid (90c, 460 mg, 972.16 μmol, 80.19% yield) as yellow solid. LCMS (ES+): m/z 399 [M+H]⁺

Step 3 (Synthesis of Compound 90d): Into a 25 mL single-necked round-bottomed flask containing a well-stirred solution of methyl 4-[4-anilino-3-(methylcarbamoyl)-6-quinolyl]-2-methyl-benzoate (89d, 200 mg, 366.65 μmol) in THF (5 mL) and water (5 mL) was added lithium hydroxide monohydrate (98% (76.93 mg, 1.83 mmol, 50.95 uL)) at room temperature. The resulting mixture was stirred at room temperature for 16 hours. After completion of reaction, volatiles were removed under vacuum and the resulting solution was acidified with 1N HCl to obtain a solid precipitate that was filtered and dried to afford 4-[4-anilino-3-(methylcarbamoyl)-6-quinolyl]-2-methyl-benzoic acid (90d, 0.15 g, 364.56 μmol, 99.43% yield). LCMS (ES+): m/z 412 [M+H]+

Step 3 (Synthesis of Compound 90f): To a stirred solution of methyl 2-[4-anilino-3-(methylcarbamoyl)-6-quinolyl]thiazole-4-carboxylate (89f, 350 mg, 836.38 μmol) in water (8 mL), THF (8 mL) and methanol (8 mL) was added lithium hydroxide monohydrate (98% (175.47 mg, 4.18 mmol, 116.21 uL)). The reaction mixture was stirred at room temperature for 12 hours. After completion of the reaction, the reaction mixture was concentrated and was acidified with 1.5N HCl solution. The resulting solid was filtered and dried to yield the product 2-[4-anilino-3-(methylcarbamoyl)-6-quinolyl]thiazole-4-carboxylic acid (90f, 100 mg, 222.53 μmol, 26.61% yield) as light yellow colored solid). LCMS (ES+): m/z 405 [M+H]+

### Alternative Synthesis for General Intermediate Scheme 6

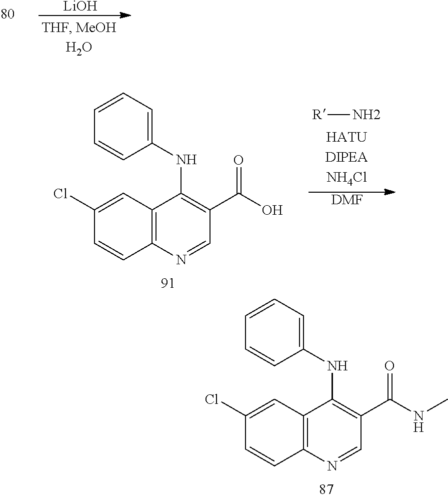

### General Intermediate Scheme 7

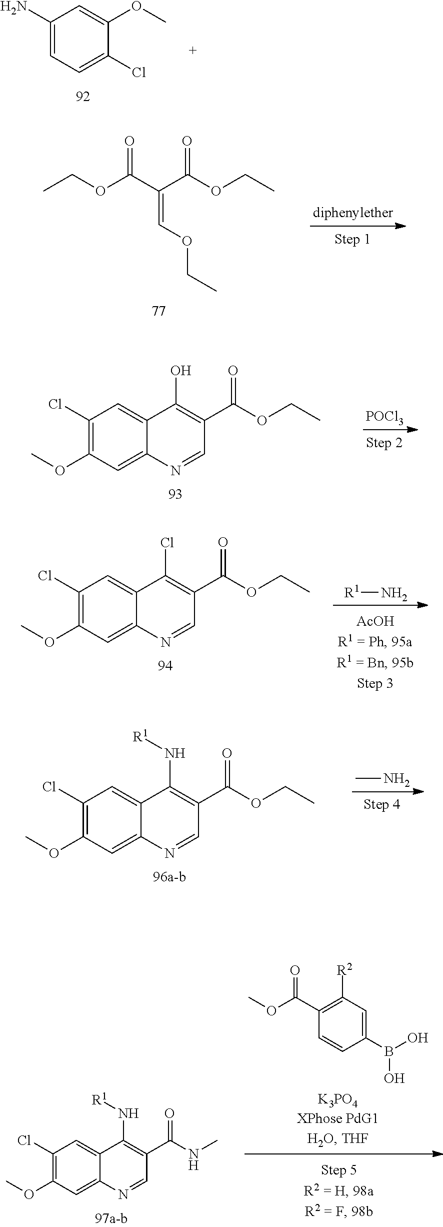

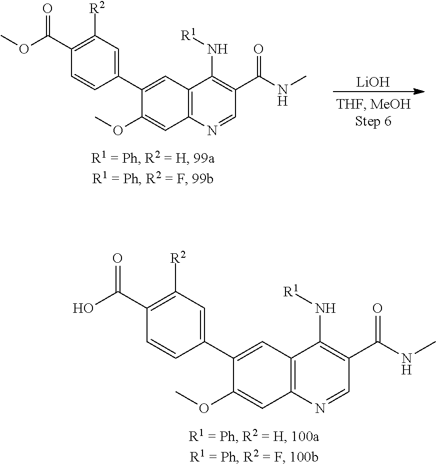

Step 1: A stirred solution of 4-chloro-3-methoxy-aniline (92, 5 g, 31.73 mmol) and diethyl 2-(ethoxymethylene)propanedioate (77, 10.29 g, 47.59 mmol, 9.53 mL) was heated to 100° C. and stirred for 2 hours and then further heated to 165° C. and stirred for an additional hour. Diphenyl ether (50.0 mL) was added to the resulting solution and the reaction was heated to 280° C. and stirred for 5 hours. The resulting reaction mixture was cooled to room temperature, pet ether (250 mL) was added and the reaction was stirred for 10 minutes at room temperature. The resulting solid was filtered, washed with pet ether and dried under vacuum to yield ethyl 6-chloro-4-hydroxy-7-methoxy-quinoline-3-carboxylate (93, 8.0 g, 28.40 mmol, 89.51% yield) as an off white solid. LCMS (ES+): m/z 282 [M+H]+

Step 2: A stirred solution of ethyl 6-chloro-4-hydroxy-7-methoxy-quinoline-3-carboxylate (93, 4 g, 14.20 mmol) in phosphorus(V) oxychloride (8.37 g, 71.00 mmol) was heated to 120° C. and the reaction was stirred for 5 hours at 120° C. The reaction was cooled to ambient temperature and concentrated under reduced pressure. The resulting crude material was dissolved in ethyl acetate and washed with 10% sodium bicarbonate solution, water and brine solution, dried over anhydrous sodium sulfate, filtered, and concentrated under reduced pressure to yield ethyl 4,6-dichloro-7-methoxy-quinoline-3-carboxylate (94, 3.5 g, 11.34 mmol, 79.83% yield) as an yellow solid. LCMS (ES+): m/z 302 [M+H]+

Step 3 (Synthesis of Compound 96a): To a stirred solution of ethyl 4,6-dichloro-7-methoxy-quinoline-3-carboxylate (94, 2 g, 6.66 mmol) and aniline (95a, 695.03 mg, 7.46 mmol, 681.40 uL) in DMF (15.0 mL) was added acetic acid (40.02 mg, 666.36 μmol, 38.11 uL) and the reaction was stirred for 1 hour at 100° C. The reaction was cooled to ambient temperature, diluted with water (50 mL) and extracted with ethyl acetate (2×25 mL). The combined organic extracts were washed with water and brine solution, dried over anhydrous sodium sulfate, filtered and concentrated under reduced pressure to yield ethyl 4-anilino-6-chloro-7-methoxy-quinoline-3-carboxylate (96a, 2 g, 5.45 mmol, 81.81% yield) as an pale yellow solid. LCMS (ES+): m/z 357 [M+H]+

Step 3 (Synthesis of Compound 96b): To a stirred solution of ethyl 4,6-dichloro-7-methoxy-quinoline-3-carboxylate (94, 0.2 g, 666.36 μmol) and phenylmethanamine (95b, 107.10 mg, 999.54 μmol) in NMP (10 mL) was added N,N-diisopropylethylamine (430.60 mg, 3.33 mmol, 580.33 uL). The reaction was heated to 100° C. overnight. The reaction mixture was concentrated and diluted with water (10 ml) and the resulting solid was filtered, washed with pet ether (30 ml) and dried under vacuum to obtain product ethyl 4-(benzylamino)-6-chloro-7-methoxy-quinoline-3-carboxylate (96b, 170 mg, 440.10 μmol, 66.04% yield) as off white colored solid. LCMS (ES+): m/z 371 [M+H]+

Step 4: An oven dried pressure tube was charged with a solution of ethyl 4-anilino-6-chloro-7-methoxy-quinoline-3-carboxylate (96a, 400 mg, 1.12 mmol) in methyl amine in methanol (1.12 mmol, 6 mL) and the reaction mixture was heated to 80° C. for 16 hours. The reaction mixture was cooled to room temperature and the reaction mixture was concentrated under reduced pressure. The crude mixture was purified by column chromatography on silica (5% methanol\dichloromethane) to yield 4-anilino-6-chloro-7-methoxy-N-methyl-quinoline-3-carboxamide (97a, 310 mg, 906.08 μmol, 80.82% yield) as yellow solid. LCMS (ES+): m/z 342 [M+H]+
Compound 97b was synthesized using the same procedure.

Alternative Synthesis of Compound 97a (not shown): To a stirred solution of ethyl 4-anilino-6-chloro-7-methoxy-quinoline-3-carboxylate (96a, 2 g, 5.61 mmol) in THF (10.0 mL) and water (10.0 mL) was added lithium hydroxide monohydrate (1.18 g, 28.03 mmol, 778.88 uL) and the reaction was stirred for 16 hours at 25° C. The resulting mixture partially concentrated under reduced pressure and the mixture was adjusted to pH of approximately 3 with saturated citric acid solution. The resulting solid was filtered, washed with water and allowed to dry under vacuum to yield 4-anilino-6-chloro-7-methoxy-quinoline-3-carboxylic acid (1.5 g, 3.47 mmol, 61.86% yield) as an off white solid. LCMS (ES+): m/z 329 [M+H]+

To a stirred solution of 4-anilino-6-chloro-7-methoxy-quinoline-3-carboxylic acid (1.5 g, 4.56 mmol) and methyl amine (8.99 g, 289.47 mmol, 10.0 mL) in DMF (10.0 mL) was added DIPEA (2.95 g, 22.81 mmol, 3.97 mL) and HATU (2.60 g, 6.84 mmol). The resulting solution was stirred for 16 hours at 25° C. The resulting mixture was diluted with water (10 mL) and extracted with ethyl acetate (3×10 mL). The combined organic extracts were washed with water and brine solution, dried over anhydrous sodium sulfate, filtered and concentrated under reduced pressure to yield 4-anilino-6-chloro-7-methoxy-N-methyl-quinoline-3-carboxamide (97a, 270 mg, 639.86 μmol, 14.02% yield). LCMS (ES+): m/z 342 [M+H]+

Step 5 (Synthesis of 99a): To a stirred solution of 4-anilino-6-chloro-7-methoxy-N-methyl-quinoline-3-carboxamide (97a, 270 mg, 789.96 μmol), (4-methoxycarbonylphenyl)boronic acid (98a, 142.16 mg, 789.96 μmol) and potassium phosphate tribasic (419.20 mg, 1.97 mmol) in THF (10 mL) and water (1.0 mL) was added X-Phos-Pd-G2 (18.64 mg, 23.70 μmol) and the reaction was stirred for 2 hours at 80° C. The resulting mixture was cooled to ambient temperature, diluted with water (15 mL) and extracted with ethyl acetate (2×20 mL). The combined organic extracts were dried over anhydrous sodium sulfate, filtered and concentrated under reduced pressure. The resulting crude material was purified by column chromatography on silica eluted with 5% methanol in dichloromethane to yield methyl 4-[4-anilino-7-methoxy-3-(methylcarbamoyl)-6-quinolyl]benzoate (99a, 280 mg, 573.09 μmol, 72.55% yield) as an pale brown solid. LCMS (ES+): m/z 442 [M+H]+

Step 5 (Synthesis of 99b): Into a 20 mL sealed tube containing a mixture of (3-fluoro-4-methoxycarbonyl-phenyl)boronic acid (98b, 76.45 mg, 386.20 μmol) and 4-anilino-6-chloro-7-methoxy-N-methyl-quinoline-3-carboxamide (97a, 110 mg, 321.83 μmol) in water (0.5 mL) and THF (4 mL) was added potassium phosphate tribasic anhydrous (170.79 mg, 804.59 μmol). Argon gas was bubbled through the reaction mixture for 10 minutes before X-Phos Precatalyst (5.06 mg, 6.44 μmol) was added and the resulting suspension was purged with argon gas for an additional 10 minutes. The contents were stirred at 80° C. under closed conditions for 2 hours at which point TLC indicated complete consumption of starting material. The reaction mixture was passed through a pad of Celite and the filtrate was concentrated under reduced pressure to afford the crude mass. The crude material was purified by silica-gel (230-400 mesh) with 7:3 EtOAc/petroleum ether to generate methyl 4-[4-anilino-7-methoxy-3-(methylcarbamoyl)-6-quinolyl]-2-fluoro-benzoate (99b, 120 mg, 261.17 μmol, 81.15% yield) as a brown solid. LCMS [460.1 (M+H)⁺].

Step 6 (Synthesis of Compound 100a): A round bottom flask was charged with methyl 4-[4-anilino-7-methoxy-3-(methylcarbamoyl)-6-quinolyl]benzoate (99a, 240 mg, 543.63 μmol) in THF (5 mL), methanol (2 mL) and water (5 mL). Lithium hydroxide powder (reagent grade (26.04 mg, 1.09 mmol)) was added and the reaction mixture was stirred for 16 hours at room temperature. The reaction mixture was concentrated under reduced pressure. The crude product was acidified with citric acid to a pH of approximately 6. The solid was filtered and dried under reduced pressure to yield 4-[4-anilino-7-methoxy-3-(methylcarbamoyl)-6-quinolyl]benzoic acid (100a, 220 mg, 511.69 μmol, 94.13% yield) as white solid. LCMS (ES+): m/z 428 [M+H]+

Step 6 (Synthesis of Compound 100b): Into a 25 mL single-necked round-bottomed flask containing a well-stirred solution of methyl 4-[4-anilino-7-methoxy-3-(methylcarbamoyl)-6-quinolyl]-2-fluoro-benzoate (99b, 120 mg, 261.17 μmol) in THF (2 mL) was added lithium hydroxide powder (reagent grade (31.27 mg, 1.31 mmol)) in water (2 mL) at room temperature. The resulting mixture was stirred at room temperature for 16 hours. The reaction mixture was concentrated under reduced pressure and the aqueous layer was acidified with saturated citric acid solution until pH=3. The resulting solid was filtered and dried to afford 4-[4-anilino-7-methoxy-3-(methylcarbamoyl)-6-quinolyl]-2-fluoro-benzoic acid (100b, 100 mg, 224.50 μmol, 85.96% yield) as a brown solid. LCMS [446.1 (M+H)⁺].

### General Intermediate Scheme 8

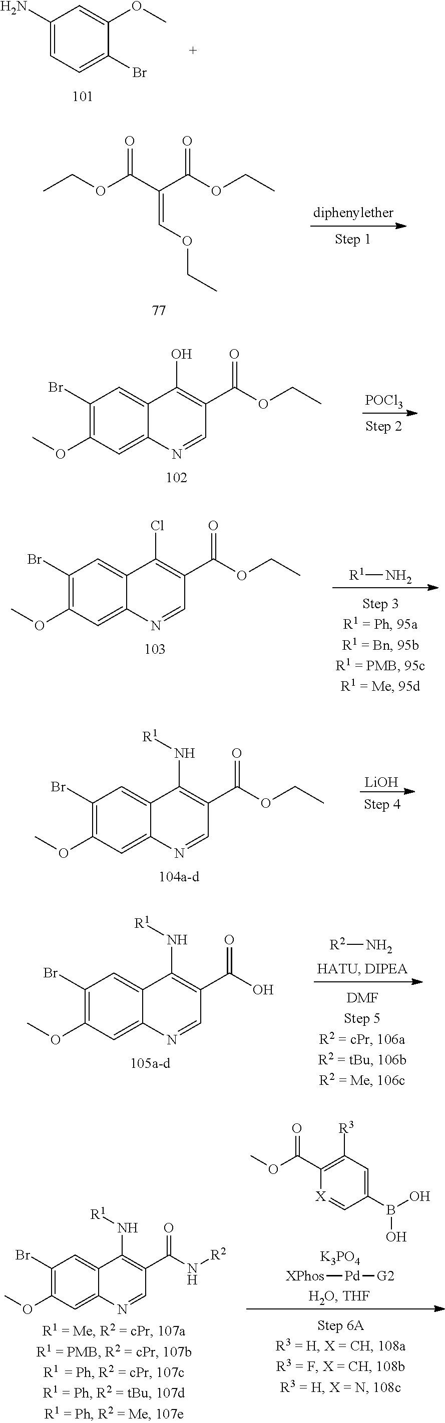 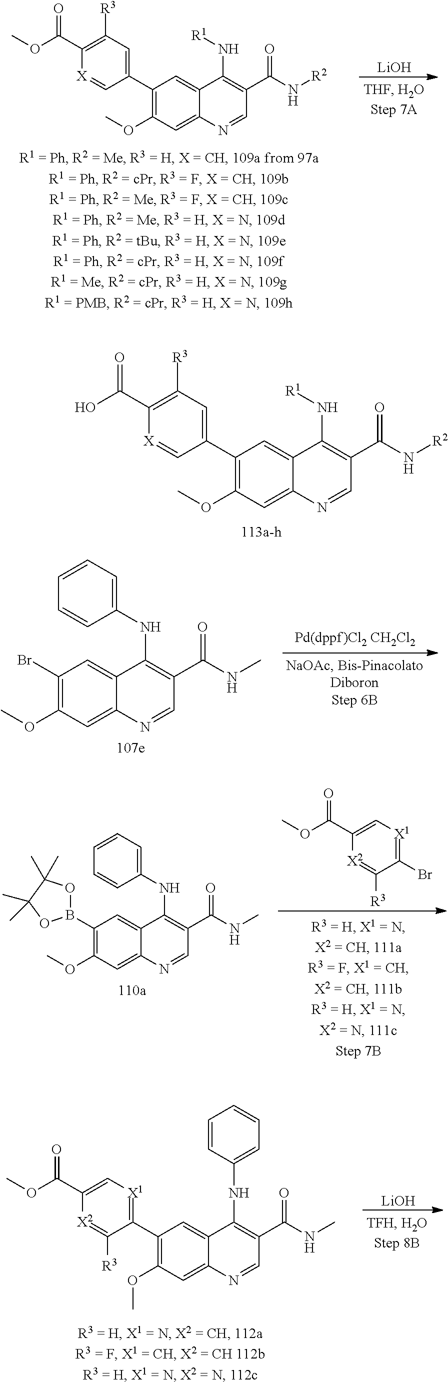 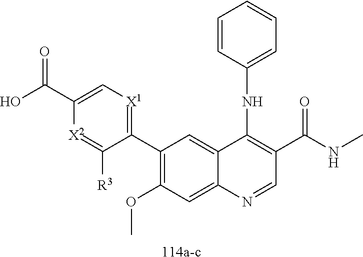 [0548] The synthesis of Compound 103 over Step 1 and Step 2 has been previously described in WO 2008/056148.

Step 3 (Synthesis of Compound 104a): Into a 100 mL sealed tube containing a mixture of ethyl 6-bromo-4-chloro-7-methoxy-quinoline-3-carboxylate (103, 2 g, 5.80 mmol) in DMF (30 mL) were added aniline (95a, 648.64 mg, 6.96 mmol, 635.92 uL) and acetic acid (348.53 mg, 5.80 mmol, 331.94 uL) under nitrogen atmosphere at room temperature. The resulting mixture was heated at 100° C. for 2 hours at which point, TLC indicated complete consumption of starting material. Ice cold water (50 mL) was added and the reaction was stirred for 10 minutes. The resulting solid was filtered and dried to afford ethyl 4-anilino-6-bromo-7-methoxy-quinoline-3-carboxylate (104a, 2.1 g, 5.23 mmol, 90.17% yield) as a yellow solid. LCMS (ES+): m/z 403 [M+H]+

Step 3 (Synthesis of Compound 104b): An oven-dried pressure tube was charged with a solution of ethyl 6-bromo-4-chloro-7-methoxy-quinoline-3-carboxylate (103, 2 g, 5.80 mmol) in DMF (20 mL), and DIPEA (3.75 g, 29.02 mmol, 5.05 mL) and benzyl amine (95b, 746.30 mg, 6.96 mmol) were added. The reaction mixture was heated to 100° C. for 2 hours and the reaction mixture was cooled to room temperature. The reaction mixture was diluted with water (30 mL) and the solid was filtered. The solid product was dried under reduced pressure to yield ethyl 4-(benzylamino)-6-bromo-7-methoxy-quinoline-3-carboxylate (104b, 2.2 g, 5.17 mmol, 89.10% yield) as white solid. LCMS (ES+): m/z 417 [M+H]+

Step 3 (Synthesis of Compound 104c): To a stirred solution of ethyl 6-bromo-4-chloro-7-methoxy-quinoline-3-carboxylate (103, 3.0 g, 8.71 mmol) in DMF (20 mL) was added DIPEA (5.63 g, 43.53 mmol, 7.58 mL) and (4-methoxyphenyl)methanamine (95c, 1.31 g, 9.58 mmol, 1.25 mL). The resulting mixture was stirred for 1 hour at 100° C. The reaction was cooled to ambient temperature and diluted with water (50 mL). The resulting solid was filtered and washed with water and allowed to dry under vacuum to yield ethyl 6-bromo-7-methoxy-4-[(4-methoxyphenyl)methylamino]quinoline-3-carboxylate (104c, 3.5 g, 7.59 mmol, 87.21% yield) as an off white solid. LCMS (ES+): m/z 446 [M+H]+

Step 3 (Synthesis of Compound 104d): To a stirred solution of ethyl 6-bromo-4-chloro-7-methoxy-quinoline-3-carboxylate (103, 1 g, 2.90 mmol) in DMF (10 mL) was added DIPEA (1.88 g, 14.51 mmol, 2.53 mL) and methylamine hydrochloride (99% (95d, 235.13 mg, 3.48 mmol)). The resulting mixture was stirred for 1 hour at 100° C. The reaction was cooled to ambient temperature and diluted with water (30 mL). The resulting solid was filtered, washed with water, and allowed to dry under vacuum to yield methyl 6-bromo-7-methoxy-4-(methylamino)quinoline-3-carboxylate (104d, 900 mg, 2.72 mmol, 93.85% yield) as an off white solid. LCMS (ES+): m/z 340 [M+H]+

Step 4 (Synthesis of Compound 105a): Into a 50 mL single-necked round-bottomed flask containing a well-stirred solution of ethyl 4-anilino-6-bromo-7-methoxy-quinoline-3-carboxylate (104a, 1 g, 2.49 mmol) in THF (5 mL) was added lithium hydroxide powder, reagent grade (298.42 mg, 12.46 mmol) in water (5 mL) at room temperature. The resulting mixture was stirred at room temperature for 16 hours. The reaction mixture was concentrated under reduced pressure and the aqueous layer was acidified with saturated citric acid solution until pH=3. The resulting solid was filtered and dried to afford 4-anilino-6-bromo-7-methoxy-quinoline-3-carboxylic acid (105a, 0.9 g, 2.41 mmol, 96.77% yield) as a yellow solid. LCMS (ES+): m/z 376 [M+H]⁺
Compound 105b was prepared using the same procedure.

Step 4 (Synthesis of Compound 105c): To a stirred solution of ethyl 6-bromo-7-methoxy-4-((4-methoxybenzyl)amino)quinoline-3-carboxylate (104c, 3.5 g, 7.86 mmol) in THF (30 mL), water (7 mL) and methanol (2 mL) was added lithium hydroxide monohydrate, 98% (1.65 g, 39.30 mmol, 1.09 mL) and the reaction was stirred for 16 hours at 25° C. The resulting mixture was partially concentrated under reduced pressure and the pH was adjusted to approximately 3 with saturated citric acid solution. The resulting solid was filtered, washed with water and allowed to dry under vacuum to yield 6-bromo-7-methoxy-4-((4-methoxybenzyl)amino)quinoline-3-carboxylic acid (105c, 3.0 g, 7.11 mmol, 90.47% yield) as an off white solid. LCMS (ES+): m/z 417 [M+H]+

Step 4 (Synthesis of Compound 105d): To a stirred solution of ethyl 6-bromo-7-methoxy-4-(methylamino)quinoline-3-carboxylate (104d, 450 mg, 1.33 mmol) in THF (7 mL), water (2 mL) and methanol (1 mL) was added lithium hydroxide, monohydrate (278.37 mg, 6.63 mmol, 184.35 uL) and the reaction was stirred for 5 hours 25° C. The resulting mixture was partially concentrated under reduced pressure and the pH was adjusted to approximately 3 with saturated citric acid solution. The resulting solid was filtered, washed with water and allowed to dry under vacuum to yield 6-bromo-7-methoxy-4-(methylamino)quinoline-3-carboxylic acid (105d, 380 mg, 1.22 mmol, 92.06% yield) as an off white solid. LCMS (ES+): m/z 312 [M+H]+

Step 5 (Synthesis of Compound 107a): To a stirred solution of 6-bromo-7-methoxy-4-(methylamino)quinoline-3-carboxylic acid (105d, 770 mg, 2.47 mmol) and cyclopropanamine (106a, 169.56 mg, 2.97 mmol, 205.78 uL) in DMF (10 mL) was added DIPEA (1.60 g, 12.37 mmol, 2.16 mL) and HATU (1.41 g, 3.71 mmol). The resulting mixture was stirred for 16 hours at 25° C. The mixture was diluted with water (20 mL) and extracted with ethyl acetate (3×25 mL). The combined organic extracts were washed with water and brine solution, dried over anhydrous sodium sulfate, filtered and concentrated under reduced pressure to yield 6-bromo-N-cyclopropyl-7-methoxy-4-(methylamino)quinoline-3-carboxamide (107a, 270 mg, 705.05 μmol, 28.49% yield) as an pale yellow semi solid. LCMS (ES+): m/z 353 [M+H]+

Step 5 (Synthesis of Compound 107b): To a stirred solution of 6-bromo-7-methoxy-4-((4-methoxybenzyl)amino)quinoline-3-carboxylic acid (105c, 2.3 g, 5.51 mmol) and cyclopropyl amine (106a, 377.66 mg, 6.61 mmol, 458.33 uL) in DMF (20 mL) was added DIPEA (3.56 g, 27.56 mmol, 4.80 mL) and HATU (3.14 g, 8.27 mmol). The resulting mixture was stirred for 16 hr at 25° C. The resulting mixture was diluted with water (50 mL) and extracted with ethyl acetate (3×25 mL). The combined organic extracts were washed with water and brine solution, dried over anhydrous sodium sulfate, filtered and concentrated under reduced pressure to yield 6-bromo-N-cyclopropyl-7-methoxy-4-((4-methoxybenzyl)amino)quinoline-3-carboxamide (107b, 1.6 g, 2.49 mmol, 45.16% yield) as an colorless solid. LCMS (ES+): m/z 458 [M+H]+

Step 5 (Synthesis of Compound 107c): Into a 50 mL two-necked round-bottomed flask containing a well-stirred solution of 4-anilino-6-bromo-7-methoxy-quinoline-3-carboxylic acid (105a, 0.6 g, 1.61 mmol) in DMF (8 mL) were added DIPEA (623.34 mg, 4.82 mmol, 840.09 uL), HATU (916.95 mg, 2.41 mmol) and cyclopropyl amine (106a, 137.69 mg, 2.41 mmol, 167.10 uL) under nitrogen atmosphere at room temperature. The resulting mixture was stirred at room temperature for 16 hours. Water (25 mL) was added and the aqueous phase was extracted twice with EtOAc (2×50 mL) to afford the crude mass. The combined organic phases were washed with brine, dried with anhydrous Na₂SO₄, filtered and concentrated under reduced pressure to afford 4-anilino-6-bromo-N-cyclopropyl-7-methoxy-quinoline-3-carboxamide (107c, 340 mg, 824.68 μmol, 51.30% yield) as a crude brown solid. LCMS (ES+): m/z 413 [M+H]+

Step 5 (Synthesis of Compound 107d): Into a 50 mL two-necked round-bottomed flask containing a well-stirred solution of 4-anilino-6-bromo-7-methoxy-quinoline-3-carboxylic acid (105a, 0.3 g, 803.86 μmol) in DMF (8 mL) were added tert-butyl amine (106b, 76.43 mg, 1.05 mmol, 109.81 uL), DIPEA (311.68 mg, 2.41 mmol, 420.05 uL) and HATU (458.48 mg, 1.21 mmol) under nitrogen atmosphere at room temperature. The resulting mixture was stirred at room temperature for 16 hours. Ice cold water (20 mL) was added and the reaction was stirred for 10 minutes. The resulting solid was filtered and dried to afford 4-anilino-6-bromo-N-tert-butyl-7-methoxy-quinoline-3-carboxamide (107d, 340 mg, 793.80 μmol, 98.75% yield) as a brown solid. LCMS (ES+): m/z 431 [M+H]+

Step 5 (Synthesis of Compound 107e): A solution of 4-anilino-6-bromo-7-methoxy-quinoline-3-carboxylic acid (105a, 201 mg, 538.59 μmol) and methylamine hydrochloride, 99% (106c, 36.36 mg, 538.59 μmol) in DMF (4.56 mL) was treated with HATU (245.74 mg, 646.30 μmol) followed by DIPEA (278.43 mg, 2.15 mmol, 375.25 uL) at room temperature. Upon completion of the reaction, as judged by LCMS, the solution was purified directly by reverse phase chromatography, eluting with 5-100% MeCN in H₂O (with 0.1% TFA modifier) to provide 4-anilino-6-bromo-7-methoxy-N-methyl-quinoline-3-carboxamide (107e) that was used without further purification. LCMS (ES+): m/z 389 [M+H]+

Alternative Step 5 (Synthesis of Compound 107e, not shown): Into a 100 mL sealed tube containing a mixture of ethyl 4-anilino-6-bromo-7-methoxy-quinoline-3-carboxylate (104a, 1 g, 2.49 mmol) in 33% methyl amine in MeOH (106c, 17.98 g, 578.94 mmol, 20 mL) was heated at 80° C. for 2 hours at which point TLC indicated complete consumption of starting material. The reaction mixture was concentrated under reduced pressure to afford 4-anilino-6-bromo-7-methoxy-N-methyl-quinoline-3-carboxamide (107e, 0.9 g, 2.33 mmol, 93.50% yield) as a brown solid. LCMS (ES+): m/z 389 [M+H]+

Step 6A (Synthesis of Compound 109a): To a stirred solution of 4-anilino-6-chloro-7-methoxy-N-methyl-quinoline-3-carboxamide (97a, 270 mg, 789.96 μmol), (4-methoxycarbonylphenyl)boronic acid (108a, 142.16 mg, 789.96 μmol) and potassium phosphate tribasic (419.20 mg, 1.97 mmol) in THF (10 mL) and water (1.0 mL) was added Xphos-Pd-G2 (18.64 mg, 23.70 μmol) and the reaction was stirred for 2 hours at 80° C. The resulting mixture was cooled ambient temperature, diluted with water (15 mL) and extracted with ethyl acetate (2×20 mL). The combined organic extracts were dried over anhydrous sodium sulfate, filtered and concentrated under reduced pressure. The resulting crude material was purified by column chromatography on silica eluted with 5% methanol in dichloromethane to yield methyl 4-[4-anilino-7-methoxy-3-(methylcarbamoyl)-6-quinolyl]benzoate (109a, 280 mg, 573.09 μmol, 72.55% yield) as an pale brown solid. LCMS (ES+): m/z 442 [M+H]+

Step 6A (Synthesis of Compound 109b): Into a 50 mL sealed tube containing a mixture of 4-anilino-6-bromo-N-cyclopropyl-7-methoxy-quinoline-3-carboxamide (107c, 1 g, 2.43 mmol) and (3-fluoro-4-methoxycarbonyl-phenyl)boronic acid (108b, 576.18 mg, 2.91 mmol) in THF (20 mL) and water (5 mL) was added potassium phosphate tribasic anhydrous (1.03 g, 4.85 mmol). Argon gas was bubbled through the reaction mixture for 5 minutes before XPhos-Pd-G2 (57.25 mg, 72.77 μmol) was added and the resulting suspension was purged with argon gas for an additional 5 minutes. The contents were heated at 80° C. for 2 ours. After completion, the reaction was filtered through a celite bed and the obtained filtrate was extracted with EtOAc two times. Excess solvent was removed from the combined organic layers to afford crude methyl 4-[4-anilino-3-(cyclopropylcarbamoyl)-7-methoxy-6-quinolyl]-2-fluoro-benzoate (109b, 1 g, 2.06 mmol, 84.92% yield) that was used in the next step without further purification. LCMS (ES+): m/z 486 [M+H]+

Step 6A (Synthesis of Compound 109c): Into a 20 mL sealed tube containing a mixture of (3-fluoro-4-methoxycarbonyl-phenyl)boronic acid (108b, 76.45 mg, 386.20 μmol) and 4-anilino-6-chloro-7-methoxy-N-methyl-quinoline-3-carboxamide (110 mg, 321.83 μmol) in water (0.5 mL) and THF (4 mL) was added potassium phosphate tribasic anhydrous (170.79 mg, 804.59 μmol). Argon gas was bubbled through the reaction mixture for 10 minutes before X-Phos Precatalyst (5.06 mg, 6.44 μmol) was added and the resulting suspension was purged with argon gas for an additional 10 minutes. The contents were stirred at 80° C. under closed condition for 2 hours at which point TLC indicated complete consumption of starting material. The reaction mixture was passed through a pad of Celite and the filtrate was concentrated under reduced pressure to afford the crude mass. The crude material was purified by a silica-gel (230-400 mesh) with 7:3 EtOAc/petroleum ether to afford methyl 4-[4-anilino-7-methoxy-3-(methylcarbamoyl)-6-quinolyl]-2-fluoro-benzoate (109c, 120 mg, 261.17 μmol, 81.15% yield) as a brown solid. LCMS [460.1 (M+H)⁺].

Step 6A (Synthesis of Compound 109d): An oven dried pressure tube was charged with a solution of 4-anilino-6-bromo-7-methoxy-N-methyl-quinoline-3-carboxamide (107e, 400 mg, 1.04 mmol) in 1,4-dioxane (8 mL) and (6-methoxycarbonyl-3-pyridyl)boronic acid (108c, 206.14 mg, 1.14 mmol) was added. The reaction mixture was purged with nitrogen for 5 minutes before Pd(dppf)Cl₂—CH₂Cl₂ (84.57 mg, 103.56 μmol) was added. The reaction mixture was heated to 90° C. for 2 hours and the reaction mixture was cooled to room temperature. The reaction mixture was diluted with water (10 mL) and the product was extracted with ethyl acetate (2×50 mL). The combined organic layers were dried over anhydrous sodium sulfate, filtered and concentrated under reduced pressure. The crude mixture was purified by column chromatography on silica (3% methanol\dichloromethane) to yield methyl 5-[4-anilino-7-methoxy-3-(methylcarbamoyl)-6-quinolyl]pyridine-2-carboxylate (109d, 450 mg, 691.27 μmol, 66.75% yield) as brown solid. LCMS (ES+): m/z 443 [M+H]+

Step 6A (Synthesis of Compound 109e): Into a 20 mL sealed tube containing a mixture of (6-methoxycarbonyl-3-pyridyl)boronic acid (108c, 50.70 mg, 280.16 μmol) and 4-anilino-6-bromo-N-tert-butyl-7-methoxy-quinoline-3-carboxamide (107d, 100 mg, 233.47 μmol) in water (0.5 mL) and dioxane (3 mL) was added potassium phosphate tribasic anhydrous (123.89 mg, 583.67 μmol). Argon gas was bubbled through the reaction mixture for 10 minutes before 1,1′-Bis(diphenylphosphino)ferrocene]dichloropalladium(II (17.08 mg, 23.35 μmol) added and again the resulting suspension was purged with argon gas for additional 10 minutes. The contents were stirred at 90° C. under closed condition. The reaction was monitored by TLC and found complete by 3 h. The reaction mixture was passed through a pad of Celite, and the filtrate was concentrated under reduced pressure to get the crude mass. The crude material was purified by a silica-gel (230-400 mesh) with 1:9 MeOH/DCM to generate methyl 5-[4-anilino-3-(tert-butylcarbamoyl)-7-methoxy-6-quinolyl]pyridine-2-carboxylate (109e, 90 mg, 185.74 μmol, 79.56% yield) as a brown gummy liquid. The desired product was characterized by LCMS [485.2 (M+H)⁺].

Step 6A (Synthesis of Compound 109f): Into a 20 mL sealed tube containing a mixture of (6-methoxycarbonyl-3-pyridyl)boronic acid (108c, 179.08 mg, 989.62 μmol) and 4-anilino-6-bromo-N-cyclopropyl-7-methoxy-quinoline-3-carboxamide (107c, 340 mg, 824.68 μmol) in water (0.5 mL) and dioxane (3 mL) was added potassium phosphate tribasic anhydrous (437.64 mg, 2.06 mmol). Argon gas was bubbled through the reaction mixture for 10 minutes before 1,1′-bis(diphenylphosphino)ferrocene]dichloropalladium(II) was added and the resulting suspension was purged with argon gas for an additional 10 minutes. The contents were stirred at 90° C. under closed condition. The reaction was monitored by TLC and found complete by 3 hours. The reaction mixture was passed through a pad of Celite and the filtrate was concentrated under reduced pressure to afford the crude mass. The crude material was purified by a silica-gel (230-400 mesh) with 1:9 MeOH/DCM to generate methyl 5-[4-anilino-3-(cyclopropylcarbamoyl)-7-methoxy-6-quinolyl]pyridine-2-carboxylate (109f, 170 mg, 362.86 μmol, 44.00% yield) as a brown gummy liquid. LCMS [469.2 (M+H)⁺].

Step 6B (Synthesis of Compound 110a): Into a 25 mL sealed tube containing a mixture of 4-anilino-6-bromo-7-methoxy-N-methyl-quinoline-3-carboxamide (107e, 0.3 g, 776.72 μmol) and 4,4,5,5-tetramethyl-2-(4,4,5,5-tetramethyl-1,3,2-dioxaborolan-2-yl)-1,3,2-dioxaborolane (256.41 mg, 1.01 mmol) in dioxane (10 mL) was added potassium acetate (190.57 mg, 1.94 mmol, 121.38 uL). Argon gas was bubbled through the reaction mixture for 10 minutes before tris(dibenzylideneacetone)dipalladium(0) (71.13 mg, 77.67 μmol) and X-PHOS (37.03 mg, 77.67 μmol) were added and the resulting suspension was purged with argon gas for an additional 10 minutes. The contents were stirred at 100° C. under closed condition for 16 hours. The reaction mixture was passed through a pad of Celite and the filtrate was concentrated under reduced pressure to afford the crude mass that was dissolved in EtOAc (100 mL) and washed with water (50 mL). The organic layer was dried with anhydrous Na₂SO₄, filtered and concentrated under reduced pressure to afford 4-anilino-7-methoxy-N-methyl-6-(4,4,5,5-tetramethyl-1,3,2-dioxaborolan-2-yl)quinoline-3-carboxamide (110a, 0.3 g, 692.35 μmol, 89.14% yield) as a crude residue. LCMS [434.2 (M+H)⁺].

Step 7B (Synthesis of Compound 112a): To a solution of 4-anilino-7-methoxy-N-methyl-6-(4,4,5,5-tetramethyl-1,3,2-dioxaborolan-2-yl)quinoline-3-carboxamide (110a, 400 mg, 923.13 μmol) and methyl 6-bromopyridine-3-carboxylate (111a, 239.31 mg, 1.11 mmol) in THF (12 mL) and water (3 mL) was added potassium phosphate tribasic anhydrous (587.86 mg, 2.77 mmol). The reaction mixture was degassed for 2 minutes followed by the addition of 2nd Generation XPhos Precatalyst (72.56 mg, 92.31 μmol) under nitrogen atmosphere at room temperature. The resulting mixture was heated at 80° C. for 4 hours. The reaction mixture was diluted with water and extracted with ethyl acetate. The combined organics were dried over anhydrous sodium sulfate and concentrated to yield crude product that was purified by column chromatography using 5% methanol in dichloromethane as eluent to yield methyl 6-[4-anilino-7-methoxy-3-(methylcarbamoyl)-6-quinolyl]pyridine-3-carboxylate (112a, 250 mg, 367.26 μmol, 39.78% yield). LCMS (ES+): m/z 443 [M+H]+

Step 7B (Synthesis of Compound 112b): To a solution of 4-anilino-7-methoxy-N-methyl-6-(4,4,5,5-tetramethyl-1,3,2-dioxaborolan-2-yl)quinoline-3-carboxamide (110a, 200 mg, 461.57 μmol) and methyl 4-bromo-3-fluoro-benzoate (111b, 161.34 mg, 692.35 μmol) in THF (8 mL) and water (2 mL) was added potassium phosphate tribasic anhydrous (293.93 mg, 1.38 mmol). The reaction mixture was degassed for 2 minutes followed by the addition of XPhos-Pd-G2 (36.32 mg, 46.16 μmol) under nitrogen atmosphere at room temperature. The resulting mixture was heated at 80° C. for 4 hours. The reaction mixture was diluted with water and extracted with ethyl acetate. The combined organics were dried over anhydrous sodium sulfate and concentrated to yield the crude product that was purified by column chromatography using 5% methanol in dichloromethane as eluent to yield methyl 4-[4-anilino-7-methoxy-3-(methylcarbamoyl)-6-quinolyl]-3-fluoro-benzoate (112b, 110 mg, 179.56 μmol, 38.90% yield). LCMS (ES+): m/z 460 [M+H]+

Step 7B (Synthesis of Compound 112c): Into a 25 mL sealed tube containing a mixture of methyl 5-bromopyrazine-2-carboxylate (111c, 150.25 mg, 692.35 μmol) and 4-anilino-7-methoxy-N-methyl-6-(4,4,5,5-tetramethyl-1,3,2-dioxaborolan-2-yl)quinoline-3-carboxamide (110a, 0.3 g, 692.35 μmol) in THF (7 mL) and water (1.5 mL) was added potassium phosphate tribasic anhydrous (367.41 mg, 1.73 mmol). Argon gas was bubbled through the reaction mixture for 10 minutes before XPhos Precatalyst (54.47 mg, 69.24 μmol) was added and the resulting suspension was purged with argon gas for an additional 10 minutes. The contents were stirred at 80° C. under closed condition for 3 hours. The reaction mixture was passed through a pad of Celite and the filtrate was concentrated under reduced pressure to afford the crude mass. The crude material was purified by a silica-gel (230-400 mesh) with 1:9 MeOH/DCM to afford methyl 5-[4-anilino-7-methoxy-3-(methylcarbamoyl)-6-quinolyl]pyrazine-2-carboxylate (112c, 170 mg, 383.35 μmol, 55.37% yield) as a brown solid. LCMS [444.2 (M+H)⁺].

Step 7A (Synthesis of Compound 113a): To a stirred solution of methyl 4-[4-anilino-7-methoxy-3-(methylcarbamoyl)-6-quinolyl]benzoate (109a, 280 mg, 634.23 μmol) in THF (5.0 mL) and water (5.0 mL) was added lithium hydroxide monohydrate, 98% (133.07 mg, 3.17 mmol, 88.13 uL) and the reaction was stirred for 16 hours at 25° C. The resulting mixture solvent was evaporated partially under reduced pressure and the pH of the mixture was adjusted to 3 (pH-3) with saturated citric acid solution. The resulting solid was filtered, washed with water and allowed to dry under vacuum to yield 4-[4-anilino-7-methoxy-3-(methylcarbamoyl)-6-quinolyl]benzoic acid (113a, 210 mg, 478.02 μmol, 75.37% yield) as an yellow solid. LCMS (ES+): m/z 428 [M+H]+

Step 7A (Synthesis of Compound 113b): Into a 100 mL single-necked round-bottomed flask containing a well-stirred solution of methyl 4-[4-anilino-3-(cyclopropylcarbamoyl)-7-methoxy-6-quinolyl]-2-fluoro-benzoate (109b, 1 g, 2.06 mmol) in THF (20 mL) and water (20 mL) was added lithium hydroxide monohydrate, 98% (432.13 mg, 10.30 mmol, 286.18 uL) at room temperature. The resulting mixture was stirred at room temperature for 16 hours. After completion, the volatiles were removed under vacuum and the resulting solution was acidified with citric acid solution to obtain a solid precipitate that was filtered and dried to afford 4-[4-anilino-3-(cyclopropylcarbamoyl)-7-methoxy-6-quinolyl]-2-fluoro-benzoic acid (113b, 0.8 g, 1.70 mmol, 82.38% yield) as an off-white solid. LCMS (ES+): m/z 472 [M+H]+

Step 7A (Synthesis of Compound 113c): Into a 25 mL single-necked round-bottomed flask containing a well-stirred solution of methyl 4-[4-anilino-7-methoxy-3-(methylcarbamoyl)-6-quinolyl]-2-fluoro-benzoate (109c, 120 mg, 261.17 μmol) in THF (2 mL) was added lithium hydroxide powder, reagent grade (31.27 mg, 1.31 mmol) in water (2 mL) at room temperature. The resulting mixture was stirred at room temperature for 16 hours. The reaction mixture was concentrated under reduced pressure and the aqueous layer was acidified with saturated citric acid solution until pH=3. The resulting solid was filtered and dried to afford 100 mg of 4-[4-anilino-7-methoxy-3-(methylcarbamoyl)-6-quinolyl]-2-fluoro-benzoic acid (113c, 100 mg, 224.50 μmol, 85.96% yield) as a brown solid. LCMS [446.1 (M+H)⁺].

Step 7A (Synthesis of Compound 113(1): A round bottom flask was charged with methyl 5-[4-anilino-7-methoxy-3-(methylcarbamoyl)-6-quinolyl]pyridine-2-carboxylate (109d, 600 mg, 1.36 mmol) in THF (10 mL), methanol (5 mL) and water (10 mL). Lithium hydroxide powder, reagent grade (64.95 mg, 2.71 mmol) was added and the reaction mixture was stirred for 16 hours at room temperature. The reaction mixture was concentrated under reduced pressure and the crude product was acidified with citric acid to a pH of approximately 6. The solid was filtered and dried over reduced pressure to yield 5-[4-anilino-7-methoxy-3-(methylcarbamoyl)-6-quinolyl]pyridine-2-carboxylic acid (113d, 490 mg, 981.85 μmol, 72.41% yield) as a black solid. LCMS (ES+): m/z 429 [M+H]+

Step 7A (Synthesis of Compound 113e): Into a 25 mL single-necked round-bottomed flask containing a well-stirred solution of methyl 5-[4-anilino-3-(tert-butylcarbamoyl)-7-methoxy-6-quinolyl]pyridine-2-carboxylate (109e, 90 mg, 185.74 μmol) in THF (2 mL) was added lithium hydroxide powder, reagent grade (22.24 mg, 928.71 μmol) in water (2 mL) at room temperature. The resulting mixture was stirred at room temperature for 16 hours. The reaction mixture was concentrated under reduced pressure and the pH of the aqueous solution was adjusted to a pH of approximately 3 with saturated citric acid. The solution was stirred for 10 minutes and the resulting solid was filtered and dried to afford 5-[4-anilino-3-(tert-butylcarbamoyl)-7-methoxy-6-quinolyl]pyridine-2-carboxylic acid (113e, 40 mg, 85.01 μmol, 45.77% yield) as a brown solid. LCMS (ES+): m/z 471 [M+H]⁺

Step 7A (Synthesis of Compound 1131): Into a 50 mL single-necked round-bottomed flask containing a well-stirred solution of methyl 5-[4-anilino-3-(cyclopropylcarbamoyl)-7-methoxy-6-quinolyl]pyridine-2-carboxylate (109f, 380 mg, 811.09 μmol) in THF (5 mL) was added lithium hydroxide powder, reagent grade (97.13 mg, 4.06 mmol) in water (5 mL) at room temperature. The resulting mixture was stirred at room temperature for 16 hours. The reaction mixture was concentrated under reduced pressure and the pH of the aqueous solution was adjusted to pH=3 with saturated citric acid. The solution was stirred for 10 minutes and the resulting solid was filtered and dried to afford 5-[4-anilino-3-(cyclopropylcarbamoyl)-7-methoxy-6-quinolyl]pyridine-2-carboxylic acid (113f, 250 mg, 550.08 μmol, 67.82% yield) as a brown solid. LCMS [455.1 (M+H)⁺].

Step 7A (Synthesis of Compound 113g): To a solution of methyl 5-[3-(cyclopropylcarbamoyl)-7-methoxy-4-(methylamino)-6-quinolyl]pyridine-2-carboxylate (109 g, 130 mg, 319.86 μmol) in THF (5 mL), water (1 mL) and methanol (1 mL) was added lithium hydroxide monohydrate (67.11 mg, 1.60 mmol, 44.44 uL) and the reaction was stirred for 16 hours at 25° C. The reaction mixture was partially concentrated under reduced pressure and the pH of the solution was adjusted to approximately 3 with saturated citric acid solution. The resulting solid was filtered and washed with water, dried under vacuum to yield 5-[3-(cyclopropylcarbamoyl)-7-methoxy-4-(methylamino)-6-quinolyl]pyridine-2-carboxylic acid (113 g, 98 mg, 149.84 μmol, 46.85% yield) as an off white solid. LCMS (ES+): m/z 393 [M+H]+

Step 7A (Synthesis of Compound 113h): To a stirred solution of methyl 5-(3-(cyclopropylcarbamoyl)-7-methoxy-4-((4-methoxybenzyl)amino)quinolin-6-yl)picolinate (109 h, 300 mg, 585.30 μmol) in THF (10 mL), water (2 mL) and methanol (1 mL) was added lithium hydroxide monohydrate, 98% (122.81 mg, 2.93 mmol, 81.33 uL) and the reaction was stirred for 16 hours at 25° C. The resulting mixture was partially concentrated under reduced pressure and the pH of the mixture was adjusted to approximately 3 with saturated citric acid solution. The resulting solid was filtered, washed with water and allowed to dry under vacuum to yield 5-(3-(cyclopropylcarbamoyl)-7-methoxy-4-((4-methoxybenzyl)amino)quinolin-6-yl)picolinic acid (113 h, 70 mg, 49.99 μmol, 8.54% yield). LCMS (ES+): m/z 499 [M+H]+

Step 8B (Synthesis of Compound 114a): To a stirred solution of methyl 6-[4-anilino-7-methoxy-3-(methylcarbamoyl)-6-quinolyl]pyridine-3-carboxylate (112a, 250 mg, 565.02 μmol) in water (5 mL), THF (5 mL) and methanol (5 mL) was added lithium hydroxide monohydrate, 98% (118.54 mg, 2.83 mmol, 78.50 uL). The reaction mixture was stirred at room temperature for 12 hours. After completion, the reaction mixture was concentrated and was acidified using 1.5N HCl solution. The resulting solid was filtered and dried to yield 6-[4-anilino-7-methoxy-3-(methylcarbamoyl)-6-quinolyl]pyridine-3-carboxylic acid (114a, 200 mg, 359.44 μmol, 63.62% yield) as light yellow colored solid. LCMS (ES+): m/z 429 [M+H]+

Step 8B (Synthesis of Compound 114b): To a stirred solution of methyl 4-[4-anilino-7-methoxy-3-(methylcarbamoyl)-6-quinolyl]-3-fluoro-benzoate (112b, 110 mg, 239.41 μmol) in water (4 mL), THF (4 mL) and methanol (4 mL) was added lithium hydroxide monohydrate, 98% (50.23 mg, 1.20 mmol, 33.26 uL). The reaction mixture was stirred at room temperature for 12 hours. After completion, the reaction mixture was concentrated and was acidified using 1.5N HCl solution. The resulting solid was filtered and dried to yield 4-[4-anilino-7-methoxy-3-(methylcarbamoyl)-6-quinolyl]-3-fluoro-benzoic acid (114b, 80 mg, 134.70 μmol, 56.26% yield) as light yellow colored solid. LCMS (ES+): m/z 446 [M+H]+

Step 8B (Synthesis of Compound 114c): Into a 25 mL single-necked round-bottomed flask containing a well-stirred solution of methyl 5-[4-anilino-7-methoxy-3-(methylcarbamoyl)-6-quinolyl]pyrazine-2-carboxylate (112c, 170.38 mg, 384.21 μmol) in THF (3 mL) was added lithium hydroxide powder, reagent grade (46.01 mg, 1.92 mmol) in water (3 mL) at room temperature. The resulting mixture was stirred at room temperature for 16 hours. The reaction mixture was concentrated under reduced pressure and the pH of the aqueous solution was adjusted to approximately 3 with saturated citric acid. The reaction was stirred for 10 minutes and the resulting solid was filtered and dried to afford 5-[4-anilino-7-methoxy-3-(methylcarbamoyl)-6-quinolyl]pyrazine-2-carboxylic acid (114c, 90 mg, 209.58 μmol, 54.55% yield) as a brown solid. LCMS [430.2 (M+H)⁺].

### General Intermediate Scheme 9

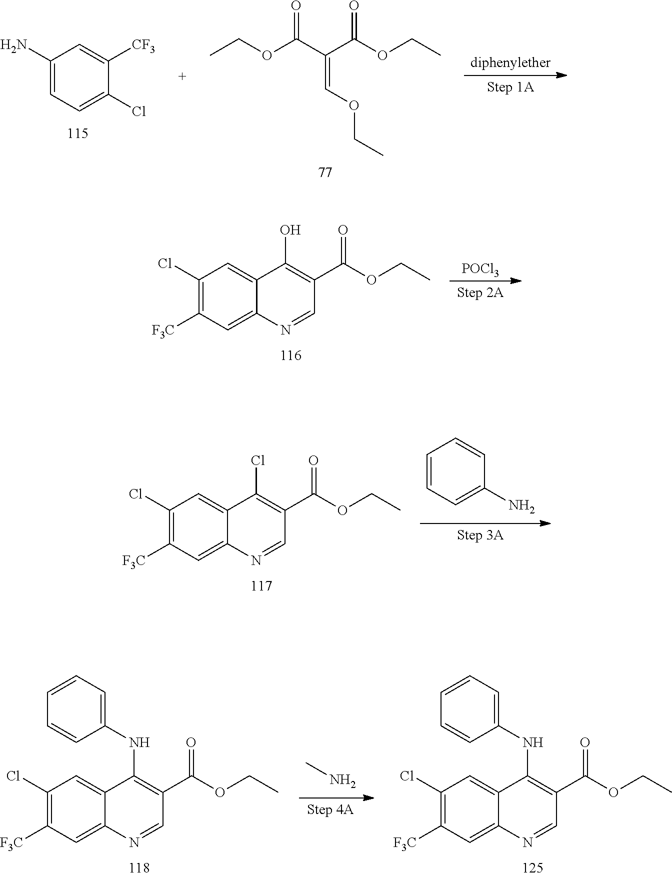

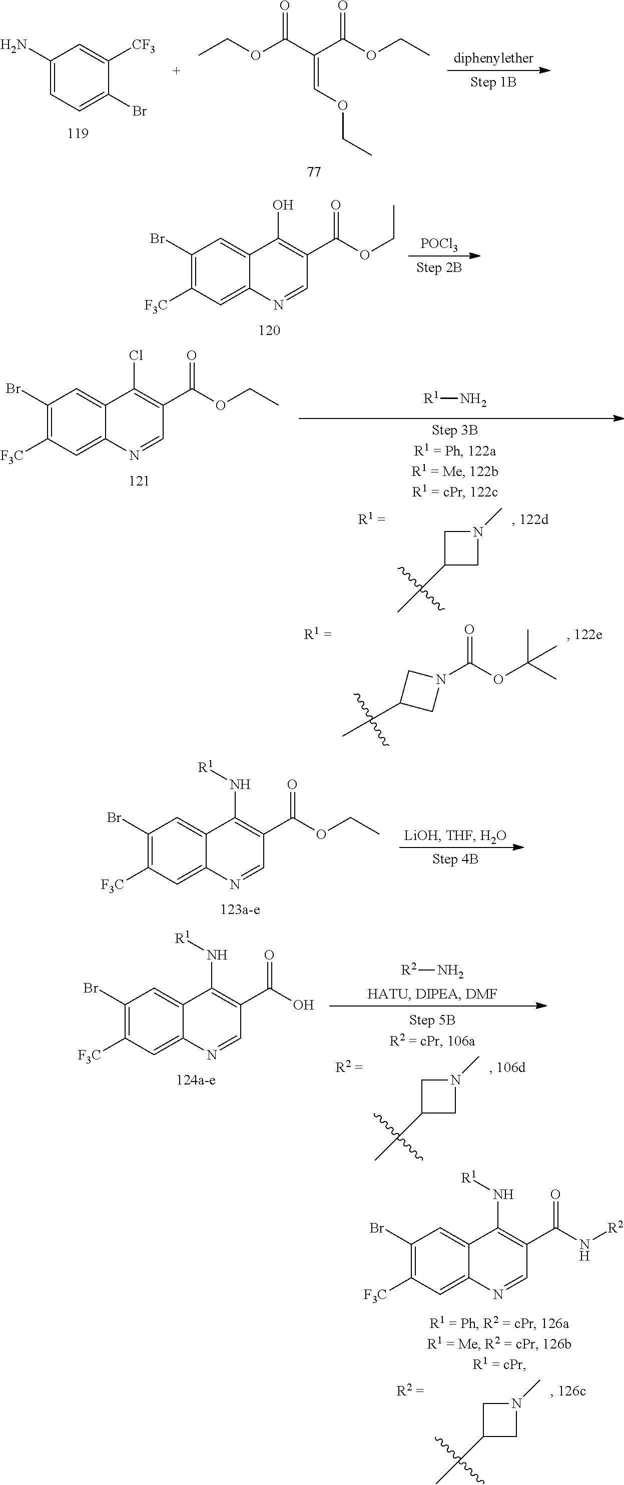

Step 1A (Synthesis of Compound 116): A mixture of 4-chloro-3-(trifluoromethyl)aniline (115, 2.0 g, 10.23 mmol) and diethyl 2-(ethoxymethylene)propanedioate (77, 3.32 g, 15.34 mmol, 3.07 mL) was stirred at 120° C. for 1 hour. The reaction mixture temperature was raised to 160° C. and stirred for 1 hour before diphenyl ether (20 mL) was added and the reaction was stirred at 280° C. for 6 hours. After completion, the reaction was allowed to cool to room temperature and was diluted with pet ether. The resulting solid precipitate was filtered and dried to obtain ethyl 6-chloro-4-hydroxy-7-(trifluoromethyl)quinoline-3-carboxylate (116, 2.8 g, 8.76 mmol, 85.65% yield) as an off-white coloured solid. LCMS (ES+): m/z 320 [M+H]+

Step 2A (Synthesis of Compound 117): An oven-dried sealed tube was charged with a solution of ethyl 6-chloro-4-hydroxy-7-(trifluoromethyl)quinoline-3-carboxylate (116, 2.8 g, 8.76 mmol) in phosphorous oxychloride (1.34 g, 8.76 mmol, 30 mL) and the reaction mixture was heated to 120° C. for 2 hours. The reaction mixture was cooled to room temperature and concentrated under reduced pressure. The crude product was diluted with water (20 mL) and extracted with ethyl acetate (2×100 mL). The combined organic layers were dried over anhydrous sodium sulphate, filtered and concentrated under reduced pressure to yield ethyl 4,6-dichloro-7-(trifluoromethyl)quinoline-3-carboxylate (117, 2.8 g, 8.28 mmol, 94.54% yield) as an off-white coloured solid. LCMS (ES+): m/z 339 [M+H]+

Step 3A (Synthesis of Compound 118): Into a 100 mL sealed tube vessel containing a well-stirred solution of ethyl 4,6-dichloro-7-(trifluoromethyl)quinoline-3-carboxylate (117, 2.8 g, 8.28 mmol) and aniline (1.16 g, 12.42 mmol, 1.13 mL) in DMF (30 mL) was added acetic acid (745.97 mg, 12.42 mmol, 710.45 uL) and the reaction was stirred at 100° C. for 2 hours. After completion, the reaction mixture was poured into ice cold water. The resulting solid precipitate was filtered and dried to obtain ethyl 4-anilino-6-chloro-7-(trifluoromethyl)quinoline-3-carboxylate (118, 3.0 g, 7.60 mmol, 91.76% yield). LCMS (ES+): m/z 395 [M+H]+

Step 4A (Synthesis of Compound 125): A 100 mL sealed tube vessel containing a well-stirred solution of ethyl 4-anilino-6-chloro-7-(trifluoromethyl)quinoline-3-carboxylate (118, 2.5 g, 6.33 mmol) and methylamine, 2M in methanol (44.95 g, 1.45 mol, 50 mL) was stirred at 80° C. for 2 hours. After completion, volatiles were removed under vacuum to obtain 4-anilino-6-chloro-N-methyl-7-(trifluoromethyl)quinoline-3-carboxamide (125, 1.3 g, 2.12 mmol, crude). LCMS (ES+): m/z 380 [M+H]⁺

Step 1B (Synthesis of Compound 120): A stirred solution of 4-bromo-3-(trifluoromethyl)aniline (119, 7 g, 29.16 mmol) and diethyl 2-(ethoxymethylene)propanedioate (77, 7.57 g, 35.00 mmol, 7.01 mL) was heated to 100° C. and stirred for 2 hours at the same temperature. The resulting mixture was further heated to 165° C. and allowed to stir for an additional 1 hour before diphenyl ether (29.16 mmol, 70 mL) was added and the reaction was raised to 280° C. and stirred for 6 hr. The mixture was cooled to ambient temperature and pet ether (500 mL) was added. The resulting solid was filtered, washed with pet ether and dried under vacuum to yield ethyl 6-bromo-4-hydroxy-7-(trifluoromethyl)quinoline-3-carboxylate (120, 7 g, 19.22 mmol, 65.92% yield) as an off white solid. LCMS (ES+): m/z 365 [M+H]+

Step 2B (Synthesis of Compound 121): A stirred solution of ethyl 6-bromo-4-hydroxy-7-(trifluoromethyl)quinoline-3-carboxylate (120, 10 g, 27.46 mmol) in POCl₃ (4.21 g, 27.46 mmol, 100 mL) was heated to 120° C. and stirred for 5 hours at the same temperature. The reaction was concentrated under reduced pressure and the resulting solid was dissolved in ethyl acetate, washed with aqueous sodium bicarbonate solution, water and brine solution, dried over anhydrous sodium sulphate, filtered and concentrated under reduced pressure to yield ethyl 6-bromo-4-chloro-7-(trifluoromethyl)quinoline-3-carboxylate (121, 6.55 g, 13.53 mmol, 49.25% yield) as an off white solid. LCMS (ES+): m/z 383 [M+H]+

Step 3B (Synthesis of Compound 123a): An oven-dried pressure tube was charged with a solution of ethyl 6-bromo-4-chloro-7-(trifluoromethyl)quinoline-3-carboxylate (121, 3 g, 7.84 mmol) in DMF (30 mL) and aniline (122a, 876.35 mg, 9.41 mmol, 859.16 uL) and acetic acid (470.92 mg, 7.84 mmol, 448.50 uL) were added. The reaction mixture was heated to 100° C. for 2 hours and then cooled to room temperature. The reaction mixture was diluted with water (60 mL) and the solid was filtered. The resulting solid was dried under vacuum to yield ethyl 4-anilino-6-bromo-7-(trifluoromethyl)quinoline-3-carboxylate (123a, 3.3 g, 7.30 mmol, 93.05% yield) as white solid. LCMS (ES+): m/z 440 [M+H]+

Step 3B (Synthesis of Compound 123b): To a stirred solution of ethyl 6-bromo-4-chloro-7-(trifluoromethyl)quinoline-3-carboxylate (121, 5 g, 13.07 mmol) and methanamine hydrochloride (122b, 1.32 g, 19.60 mmol) in DMF (50 mL) was added DIPEA (5.07 g, 39.21 mmol, 6.83 mL) The reaction was stirred for 3 hours at 100° C. and then cooled to ambient temperature. The reaction mixture was concentrated to remove DMF and the resulting solid was washed with water and extracted with EtOAc (200×2) mL. The combined organic layers were washed with brine, dried over Na₂SO₄, filtered and concentrated under reduced pressure to obtain crude product ethyl 6-bromo-4-(methylamino)-7-(trifluoromethyl)quinoline-3-carboxylate (123b, 4.2 g, 10.01 mmol, 92% yield) as a white solid.

Step 3B (Synthesis of Compound 123c): To a stirred solution of ethyl 6-bromo-4-chloro-7-(trifluoromethyl)quinoline-3-carboxylate (121, 4.2 g, 10.98 mmol) and cyclopropanamine (122c, 626.82 mg, 10.98 mmol, 760.70 uL) was added N,N-diisopropylethylamine (7.09 g, 54.89 mmol, 9.56 mL) and the reaction was stirred at 80° C. for 8 hours. The progress of the reaction was monitored by TLC and LC-MS. The reaction mixture was cooled to ambient temperature and diluted with ice-cooled water. The resulting precipitate was filtered, washed with pet ether and dried to afford ethyl 6-bromo-4-(cyclopropylamino)-7-(trifluoromethyl)quinoline-3-carboxylate (123c, 3.7 g, 9.12 mmol, 83.04% yield) as a pale yellow solid. LCMS (ES+): m/z 404 [M+H]+

Step 3B (Synthesis of Compound 123(1): To a stirred solution of ethyl 6-bromo-4-chloro-7-(trifluoromethyl)quinoline-3-carboxylate (121, 1.5 g, 3.92 mmol) in DMF (15 mL) was added DIPEA (2.53 g, 19.60 mmol, 3.41 mL) and 1-methylazetidin-3-amine (122d, 371.51 mg, 4.31 mmol). The resulting mixture was heated to 100° C. and stirred for 1 hour at the same temperature. The reaction was then cooled to ambient temperature and water (50 mL) was added. The resulting solid was filtered, washed with water and allowed to dry under vacuum to yield ethyl 6-bromo-4-[(1-methylazetidin-3-yl)amino]-7-(trifluoromethyl)quinoline-3-carboxylate (123d, 1.5 g, 2.57 mmol, 65.50% yield) as an off white solid. LCMS (ES+): m/z 432 [M+H]+

Step 3B (Synthesis of Compound 123e): Into a 50 mL sealed tube containing a mixture of tert-butyl 3-aminoazetidine-1-carboxylate (122e, 540.23 mg, 3.14 mmol) in DMF (15 mL) were added DIPEA (1.69 g, 13.07 mmol, 2.28 mL) and ethyl 6-bromo-4-chloro-7-(trifluoromethyl)quinoline-3-carboxylate (121, 540.23 mg, 3.14 mmol) under nitrogen atmosphere at room temperature. The resulting mixture was heated at 100° C. for 2 hours at which point TLC indicated complete consumption of starting material. Ice cold water (50 mL) was added and the reaction was stirred for 10 minutes. The resulting solid was filtered and dried to afford ethyl 6-bromo-4-[(1-tert-butoxycarbonylazetidin-3-yl)amino]-7-(trifluoromethyl)quinoline-3-carboxylate (123e, 1.3 g, 2.51 mmol, 95.95% yield) as an off-white-solid. LCMS (ES+): m/z 519 [M+H]+

Step 4B (Synthesis of Compound 124a): Into a 50 mL single-necked round-bottomed flask containing a well-stirred solution of ethyl 4-anilino-6-bromo-7-(trifluoromethyl)quinoline-3-carboxylate (123a, 0.8 g, 1.82 mmol) in methanol (5 mL) was added lithium hydroxide powder, reagent grade (218.11 mg, 9.11 mmol) in water (5 mL) at room temperature. The resulting mixture was stirred at room temperature for 16 hours. The reaction mixture was concentrated under reduced pressure and the aqueous solution was adjusted to pH=3 with saturated citric acid. The reaction was stirred for 10 minutes and the resulting solid was filtered and dried to afford 4-anilino-6-bromo-7-(trifluoromethyl)quinoline-3-carboxylic acid (124a, 650 mg, 1.58 mmol, 86.79% yield) as a brown solid. LCMS (ES+): m/z 411 [M+H]+

Step 4B (Synthesis of Compound 124b): To a stirred solution of ethyl 6-bromo-4-(methylamino)-7-(trifluoromethyl)quinoline-3-carboxylate (123b, 1.7 g, 4.51 mmol) in THF (25 mL), methanol (2 mL) and water (5 mL) was added lithium hydroxide monohydrate (945.74 mg, 22.54 mmol, 626.32 uL) and the reaction was stirred for 5 hours at 25° C. The reaction mixture was partially concentrated under reduced pressure and the pH of the mixture was adjusted to approximately 3 with saturated citric acid solution. The resulting solid was filtered, washed with water and allowed to dry under vacuum to yield 6-bromo-4-(methylamino)-7-(trifluoromethyl)quinoline-3-carboxylic acid (124b, 1.55 g, 4.42 mmol, 98.11% yield) as an off white solid. LCMS (ES+): m/z 350 [M+H]+

Step 4B (Synthesis of Compound 124c): To a stirred solution of ethyl 6-bromo-4-(cyclopropylamino)-7-(trifluoromethyl)quinoline-3-carboxylate (123c, 3.7 g, 9.18 mmol) in THF (40 mL), water (40 mL), and ethanol (40 mL) was added lithium hydroxide powder, reagent grade (1.10 g, 45.88 mmol) at 0° C. and the reaction was stirred at ambient temperature for 8 hours. The progress of the reaction was monitored by TLC and LC-MS. The reaction mixture was partially concentrated and the pH was adjusted to approximately 3 with saturated citric acid solution. The resulting solid was filtered, washed with water and allowed to dry under vacuum to yield 6-bromo-4-(cyclopropylamino)-7-(trifluoromethyl)quinoline-3-carboxylic acid (124c, 2.9 g, 7.61 mmol, 82.98% yield) as an off white solid. LCMS (ES+): m/z 376 [M+H]+

Step 4B (Synthesis of Compound 124d): To a stirred solution of ethyl 6-bromo-4-[(1-methylazetidin-3-yl)amino]-7-(trifluoromethyl)quinoline-3-carboxylate (123d, 1.2 g, 2.78 mmol) in THF (20 mL), water (5 mL) and methanol (1 mL) was added lithium hydroxide monohydrate, 98% (582.51 mg, 13.88 mmol, 385.77 uL) and the reaction was stirred for 16 hours at 25° C. The resulting mixture solvent was partially concentrated under reduced pressure and the pH of the mixture was adjusted to approximately 3 with saturated citric acid solution. The resulting solid was filtered, washed with water and allowed to dry under vacuum to yield 6-bromo-4-[(1-methylazetidin-3-yl)amino]-7-(trifluoromethyl)quinoline-3-carboxylic acid (124d, 1.0 g, 2.30 mmol, 82.88% yield) as an off white solid. LCMS (ES+): m/z 405 [M+H]+

Step 4B (Synthesis of Compound 124e): Into a 50 mL single-necked round-bottomed flask containing a well-stirred solution of ethyl 6-bromo-4-[(1-tert-butoxycarbonylazetidin-3-yl)amino]-7-(trifluoromethyl)quinoline-3-carboxylate (123e, 1 g, 1.93 mmol) in water (5 mL) was added lithium hydroxide powder, reagent grade (231.03 mg, 9.65 mmol) in THF (5 mL) at room temperature. The resulting mixture was stirred at room temperature for 16 hours. The reaction mixture was concentrated under reduced pressure and the aqueous layer was acidified with saturated citric acid solution until pH=3. The resulting solid was filtered and dried to afford 6-bromo-4-[(1-tert-butoxycarbonylazetidin-3-yl)amino]-7-(trifluoromethyl)quinoline-3-carboxylic acid (124e, 0.9 g, 1.84 mmol, 95.15% yield) as a brown solid. LCMS (ES+): m/z 491 [M+H]⁺

Step 5B (Synthesis of Compound 126a): To an oven-dried round bottom flask was charged with a solution of 4-anilino-6-bromo-7-(trifluoromethyl)quinoline-3-carboxylic acid (124a, 2.9 g, 7.05 mmol) in DMF (30 mL), DIPEA (4.56 g, 35.27 mmol, 6.14 mL), HATU (3.22 g, 8.46 mmol) and cyclopropylamine (106a, 1.21 g, 21.16 mmol, 1.47 mL) were added. The reaction mixture was stirred for 16 hours at room temperature. The reaction mixture was diluted with water (60 mL) and the resulting solid was filtered. The solid product was dried under vacuum to yield 4-anilino-6-bromo-N-cyclopropyl-7-(trifluoromethyl)quinoline-3-carboxamide (126a, 3 g, 2.33 mmol, 91% yield) as yellow solid. LCMS (ES+): m/z 451 [M+H]+

Step 5B (Synthesis of Compound 126b): To a stirred solution of 6-bromo-4-(methylamino)-7-(trifluoromethyl)quinoline-3-carboxylic acid (124b, 1.55 g, 4.44 mmol) and cyclopropanamine (106a, 380.24 mg, 6.66 mmol, 461.46 uL) in DMF (25 mL) was added DIPEA (2.87 g, 22.20 mmol, 3.87 mL) and HATU (2.53 g, 6.66 mmol). The resulting mixture was stirred for 16 hours at 25° C. The resulting mixture was diluted with water (50 mL) and extracted with ethyl acetate (3×25 mL). The combined organic extracts were washed with water and brine solution, dried over anhydrous sodium sulphate, filtered and concentrated under reduced pressure. The resulting crude material was purified by column chromatography on silica eluted with 10% methanol in dichloromethane to yield 6-bromo-N-cyclopropyl-4-(methylamino)-7-(trifluoromethyl)quinoline-3-carboxamide (126b, 970 mg, 1.85 mmol, 41.65% yield) as an off white solid. LCMS (ES+): m/z 389 [M+H]+

Step 5B (Synthesis of Compound 126c): To a stirred solution of 6-bromo-4-(cyclopropylamino)-7-(trifluoromethyl)quinoline-3-carboxylic acid (124c, 500 mg, 1.33 mmol) and 1-methylazetidin-3-amine (106d, 172.21 mg, 2.00 mmol) in DMF (20 mL) was added DIPEA (0.5 g, 3.87 mmol, 673.85 uL) and (benzotriazol-1-yloxy)tripyrrolidinophosphonium hexafluorophosphate (1.04 g, 2.00 mmol). The resulting mixture was stirred at ambient temperature for 16 hours. The progress of the reaction was monitored by TLC and LC-MS. The reaction mixture was diluted with water (30 mL) and extracted with ethyl acetate (3×50 mL). The combined organic extracts were washed with water and brine solution, dried over anhydrous sodium sulphate, filtered and concentrated under reduced pressure to yield 6-bromo-4-(cyclopropylamino)-N-(1-methylazetidin-3-yl)-7-(trifluoromethyl)quinoline-3-carboxamide (126c, 0.3 g, 648.38 umol, 48.65% yield) an pale brown oil. LCMS (ES+): m/z 444 [M+H]+

### General Intermediate Scheme 10

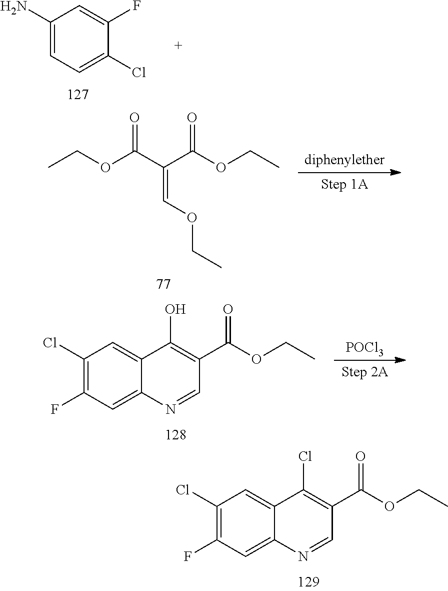

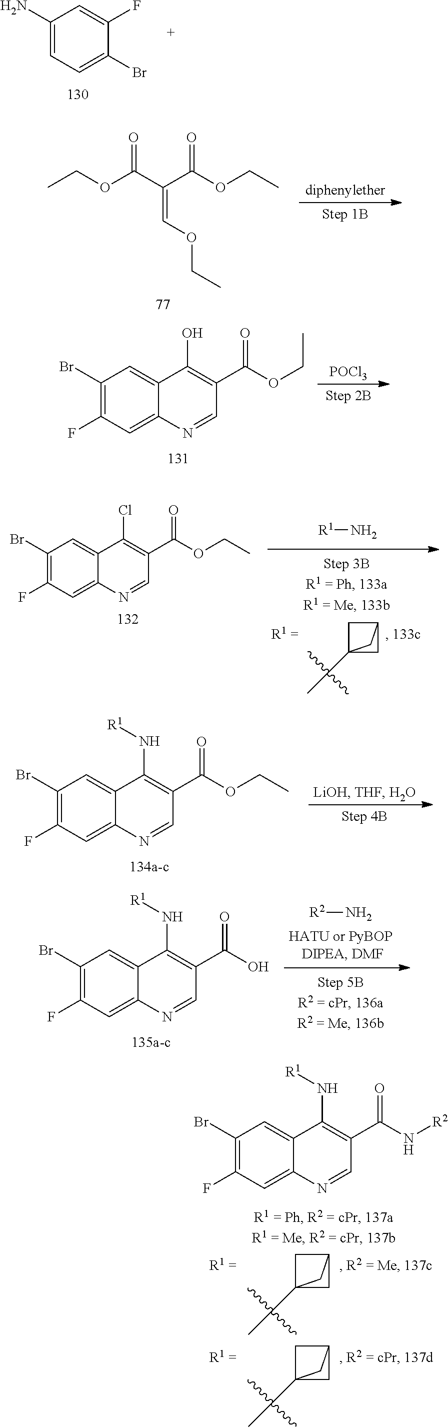

Step 1A (Synthesis of Compound 128): The mixture of 4-chloro-3-fluoro-aniline (127, 3.0 g, 20.61 mmol) and diethyl ethoxymethylenemalonate (77, 6.68 g, 30.91 mmol, 6.19 mL) was stirred at 120° C. for 1 hour. The reaction mixture temperature was raised to 160° C. and stirred for 1 hour. Then diphenyl ether (30 mL) was added and the reaction was stirred at 280° C. for 6 hours. After completion, the reaction was allowed to warm to room temperature and was diluted with pet ether. The resulting solid precipitate was filtered and dried to obtain ethyl 6-chloro-7-fluoro-4-hydroxy-quinoline-3-carboxylate (128, 3.0 g, 11.13 mmol, 53.98% yield) as an off-white colored solid. LCMS (ES+): m/z 270 [M+H]+

Step 2A (Synthesis of Compound 129): An oven-dried pressure tube was charged with a solution of ethyl 6-chloro-7-fluoro-4-hydroxy-quinoline-3-carboxylate (128, 3.0 g, 11.13 mmol) and phosphorus(V) oxychloride (1.31 g, 8.55 mmol, 30 mL) and the reaction mixture was heated to 120° C. for 2 hours. The reaction mixture was cooled to room temperature and concentrated under reduced pressure. The crude product was diluted with water (20 mL) and extracted with ethyl acetate (2×100 mL). The combined organic layers were dried over anhydrous sodium sulfate, filtered and concentrated under reduced pressure to yield ethyl 4,6-dichloro-7-fluoro-quinoline-3-carboxylate (129, 3.1 g, 10.76 mmol, 96.72% yield) as yellow solid. LCMS (ES+): m/z 289 [M+H]+

Step 1B (Synthesis of Compound 131): A three-neck round bottom flask was charged with 4-bromo-3-fluoroaniline (130, 120 g, 631.54 mmol) and diethyl 2-(ethoxymethylene)malonate (77, 140.65 g, 650.48 mmol) was added. The reaction as stirred at 100° C. for 30 minutes and then at 165° C. for 30 minutes. Diphenylether (600 mL) was added and the resulting mixture was stirred at 240° C. for 3 hours. The reaction was then cool to room temperature and allowed to stir overnight before EtOH (29 mL) was added. The reaction was heated to 250° C. and stirred for 30 minutes before EtOH (33 mL) was added. The reaction was cooled to 60° C. with a water bath. The reaction was diluted with acetone (600 mL), cooled to 20° C., stirred for 30 minutes, filtered, and washed with acetone (600 mL) and MTBE (600 mL×2), and dried by air flow to afford ethyl 6-bromo-7-fluoro-4-hydroxyquinoline-3-carboxylate (131, 137.7 g, 438.39 mmol, 69.4% yield) LCMS (ES+): m/z 315 [M+H]+

Step 2B (Synthesis of Compound 132): To a solution of ethyl 6-bromo-7-fluoro-4-hydroxyquinoline-3-carboxylate (131, 137.7 g, 438.9 mmol) in toluene (960 mL) was added POCl₃ (105.6 g, 688.71 mmol, 64 mL). The reaction was stirred at 100° C. overnight at which point the reaction was complete by LC-MS. The reaction was cooled to 8° C. with an ice bath. The temperature was then further decreased to −3° C. and aqueous K₂CO₃ solution (600 mL) was slowly added. EtOAc (960 mL) was added and the solution was stirred for 20 minutes. The organic layer was washed with water (550 mL) and concentrated. The resulting residue was stirred in DCM (550 mL) with SiO2 (0.5×) for 30 minutes and the SiO2 (0.5×) was filtered. The crude material was purified via flash column chromatography with DCM/hex/EtOAc (1.8 L, 1:1:1) to yield ethyl 6-bromo-4-chloro-7-fluoroquinoline-3-carboxylate (132, 142 g, 427 mmol, 97.4% yield) LCMS (ES+): m/z 315 [M+H]+

Step 3B (Synthesis of Compound 134a): An oven-dried pressure tube was charged with a solution of ethyl 6-bromo-4-chloro-7-fluoro-quinoline-3-carboxylate (132, 5 g, 15.04 mmol) in DMF (50 mL) and aniline (133a, 1.68 g, 18.04 mmol, 1.65 mL) and acetic acid (902.86 mg, 15.04 mmol, 859.87 uL) were added. The reaction mixture was heated to 100° C. for 2 hours and then cooled to room temperature. The reaction mixture was diluted with water (100 mL) and the solid was filtered. The solid product was dried under vacuum to yield ethyl 4-anilino-6-bromo-7-fluoro-quinoline-3-carboxylate (134a, 4.9 g, 9.70 mmol, 64.53% yield) as a yellow solid. LCMS (ES+): m/z 390 [M+H]+

Step 3B (Synthesis of Compound 134b): An oven dried pressure tube was charged with a solution of ethyl 6-bromo-4-chloro-7-fluoro-quinoline-3-carboxylate (132, 5 g, 15.04 mmol) in DMF (50 mL) and methylamine hydrochloride, 99% (133b, 1.52 g, 22.55 mmol) and DIPEA (9.72 g, 75.18 mmol, 13.09 mL) were added. The reaction mixture was heated to 100° C. for 3 hours and the reaction mixture was cooled to room temperature. The reaction mixture was diluted with water and the product was extracted with ethyl acetate. The combined organic layer was dried over anhydrous sodium sulfate, filtered and concentrated under reduced pressure to afford ethyl 6-bromo-7-fluoro-4-(methylamino)quinoline-3-carboxylate (134b, 4.8 g, 13.94 mmol, 92.71% yield). LCMS (ES+): m/z 328 [M+H]+

Step 3B (Synthesis of Compound 134c): To a solution of ethyl 6-bromo-4-chloro-7-fluoro-quinoline-3-carboxylate (132, 600 mg, 1.80 mmol) and bicyclo[1.1.1]pentan-3-amine (133c, 164.99 mg, 1.98 mmol, 021) in DMF (10 mL) was added DIPEA (1.17 g, 9.02 mmol, 1.57 mL) and the reaction was stirred for 1 hour at 100° C. The reaction was cooled to ambient temperature, diluted with water (20 mL) and extracted with ethyl acetate (3×15 mL). The combined organic extracts were washed with water and brine solution, dried over anhydrous sodium sulfate, filtered and concentrated under reduced pressure. The resulting crude was purified by column chromatography on silica eluted with 0-30% ethyl acetate in pet ether to yield ethyl 4-(3-bicyclo[1.1.1]pentanylamino)-6-bromo-7-fluoro-quinoline-3-carboxylate (134c, 570 mg, 1.42 mmol, 78.76% yield). LCMS (ES+): m/z 380 [M+H]+

Step 4B (Synthesis of Compound 135a): A round bottom flask was charged with ethyl 4-anilino-6-bromo-7-fluoro-quinoline-3-carboxylate (134a, 4.9 g, 12.59 mmol) in THF (20 mL), methanol (15 mL) and water (20 mL) and lithium hydroxide powder, reagent grade (603.03 mg, 25.18 mmol) was added. The reaction mixture was stirred for 16 hours at room temperature and then concentrated under reduced pressure. The crude product was acidified with citric acid up to a pH of approximately 6. The solid was filtered and dried under vacuum to yield 4-anilino-6-bromo-7-fluoro-quinoline-3-carboxylic acid (135a, 4.5 g, crude) as yellow solid. LCMS (ES+): m/z 362 [M+H]+

Step 4B (Synthesis of Compound 135b): To an oven dried single-necked round-bottomed flask containing a well-stirred solution of ethyl 6-bromo-7-fluoro-4-(methylamino)quinoline-3-carboxylate (134b, 5.5 g, 16.81 mmol) in THF/H₂O (20.00 mL) and ethanol (5 mL) was added lithium hydroxide (2.01 g, 84.06 mmol) and the resulting mixture was stirred at room temperature for 16 hours. After completion, the volatiles were removed under vacuum and the resulting solution was acidified with citric acid solution to obtain solid precipitate that was filtered and dried to afford 6-bromo-7-fluoro-4-(methylamino)quinoline-3-carboxylic acid (135b, 5 g, crude) as a white color solid. LCMS (ES+): m/z 300 [M+H]+

Step 4B (Synthesis of Compound 135c): To a solution of ethyl 4-(3-bicyclo[1.1.1]pentanylamino)-6-bromo-7-fluoro-quinoline-3-carboxylate (134c, 1.8 g, 4.75 mmol) in THF (10 mL) and methanol (1 mL) was added a solution of lithium hydroxide monohydrate (597.55 mg, 14.24 mmol, 395.73 uL) in water (2 mL) and the reaction was stirred for 16 hours at room temperature. The reaction was partially concentrated under reduced pressure and the pH was adjusted to approximately 3 with saturated citric acid solution. The resulting solid was filtered, washed with water, and dried under vacuum to yield 4-(3-bicyclo[1.1.1]pentanylamino)-6-bromo-7-fluoro-quinoline-3-carboxylic acid (135c, 1.5 g, 3.84 mmol, 80.99% yield). LCMS (ES+): m/z 352 [M+H]+

Step 5B (Synthesis of Compound 137a): To a stirred solution of 4-anilino-6-bromo-7-fluoro-quinoline-3-carboxylic acid (135a, 800 mg, 2.22 mmol) in DMF (15 mL) was added cyclopropylamine (136a, 189.70 mg, 3.32 mmol, 230.22 uL), N,N-diisopropylethylamine (1.43 g, 11.08 mmol, 1.93 mL) and PyBOP (1.73 g, 3.32 mmol). The reaction mixture was stirred for 16 hours at 25° C. The reaction mixture was diluted with water (10 mL) and extracted with ethyl acetate (3×25 mL). The combined organic extracts were washed with water and brine solution, dried over anhydrous sodium sulfate, filtered and concentrated under reduced pressure. The resulting crude material was purified by column by using 80% ethyl acetate in pet ether as eluent to yield pure product 4-anilino-6-bromo-N-cyclopropyl-7-fluoro-quinoline-3-carboxamide (137a, 750 mg, 1.87 mmol, 84.60% yield) as a light brown colored gummy solid. LCMS (ES+): m/z 401 [M+H]+

Step 5B (Synthesis of Compound 137b): An oven-dried round bottom flask was charged with 6-bromo-7-fluoro-4-(methylamino)quinoline-3-carboxylic acid (135b, 5.00 g, 16.72 mmol), cyclopropylamine (136a, 1.43 g, 25.08 mmol, 1.74 mL) and HATU (9.53 g, 25.08 mmol). The reaction mixture was stirred for 16 hours at room temperature. The reaction mixture was diluted with water (30 mL) and extracted with ethyl acetate (3×25 mL). The combined organic extracts were washed with water and brine solution, dried over anhydrous sodium sulfate, filtered and concentrated under reduced pressure to afford 6-bromo-N-cyclopropyl-7-fluoro-4-(methylamino)quinoline-3-carboxamide (137b, 5 g, crude) as a white colored solid. LCMS (ES+): m/z 339 [M+H]+

Step 5B (Synthesis of Compound 137c): To a solution of 4-(3-bicyclo[1.1.1]pentanylamino)-6-bromo-7-fluoro-quinoline-3-carboxylic acid (135c, 400 mg, 1.14 mmol) in DMF (10 mL) was added DIPEA (1.47 g, 11.39 mmol, 1.98 mL) and HATU (649.65 mg, 1.71 mmol). The resulting mixture was stirred for 10 minutes at room temperature and methylamine hydrochloride (136b, 230.72 mg, 3.42 mmol, 690.58 uL) was added. The reaction was stirred for 16 hours at 25° C. The resulting mixture was diluted with water (20 mL) and extracted with ethyl acetate (3×20 mL). The combined organic extracts were washed with water and brine solution, dried over anhydrous sodium sulfate, filtered and concentrated under reduced pressure. The resulting crude was purified by column chromatography on silica eluted with 5% methanol in dichloromethane to yield 4-(3-bicyclo[1.1.1]pentanylamino)-6-bromo-7-fluoro-N-methyl-quinoline-3-carboxamide (137c, 340 mg, 907.85 μmol, 79.70% yield) as an off white solid. LCMS (ES+): m/z 365 [M+H]+

Step 5B (Synthesis of Compound 137(1): To a solution of 4-(3-bicyclo[1.1.1]pentanylamino)-6-bromo-7-fluoro-quinoline-3-carboxylic acid (135c, 700 mg, 1.99 mmol) in DMF (10 mL) was added DIPEA (2.58 g, 19.93 mmol, 3.47 mL) and HATU (1.14 g, 2.99 mmol). The resulting mixture was stirred for 10 minutes at room temperature and cyclopropylamine (136a, 569.04 mg, 9.97 mmol, 690.58 uL) was added. The reaction was stirred for 16 hours at 25° C. The resulting mixture was diluted with water (20 mL) and extracted with ethyl acetate (3×20 mL). The combined organic extracts were washed with water and brine solution, dried over anhydrous sodium sulfate, filtered and concentrated under reduced pressure. The resulting crude was purified by column chromatography on silica eluted with 5% methanol in dichloromethane to yield 4-(3-bicyclo[1.1.1]pentanylamino)-6-bromo-N-cyclopropyl-7-fluoro-quinoline-3-carboxamide (137d, 570 mg, 1.27 mmol, 63.50% yield) as an off white solid. LCMS (ES+): m/z 391 [M+H]+

Step 6B (Synthesis of Compound 139a): An oven dried pressure tube was charged with a solution of 4-anilino-6-bromo-N-cyclopropyl-7-fluoro-quinoline-3-carboxamide (137a, 2.7 g, 6.75 mmol) in Dioxane (30 mL) Water (10 mL), (3-fluoro-4-methoxycarbonyl-phenyl)boronic acid (138a, 1.60 g, 8.10 mmol) and Potassium phosphate tribasic anhydrous (2.15 g, 10.12 mmol) were added. The reaction mixture was purged with nitrogen for 5 minutes, Pd(dppf)Cl2.CH2Cl2 (550.90 mg, 674.59 μmol) was added. The reaction mixture was heated to 80° C. for 1.5 h and the reaction mixture was cooled to room temperature. The reaction mixture was diluted with water (30 mL) and the product was extracted with ethyl acetate (2×100 mL). The combined organic layer was dried over anhydrous sodium sulfate, filtered and concentrated under reduced pressure. The crude mixture was purified by column chromatography on silica (3% Methanol/Ethyl acetate) to yield methyl 4-[4-anilino-3-(cyclopropylcarbamoyl)-7-fluoro-6-quinolyl]-2-fluoro-benzoate (139a, 2.4 g, 4.30 mmol, 63.71% yield, 84.79% purity) as yellow solid. LCMS (ES+): m/z 474 [M+H]+

Step 6B (Synthesis of Compound 139b): Into a 50 mL sealed tube containing a mixture 6-bromo-N-cyclopropyl-7-fluoro-4-(methylamino)quinoline-3-carboxamide (137b, 5 g, 14.79 mmol), (3-fluoro-4-methoxycarbonyl-phenyl)boronic acid (138b, 4.39 g, 22.18 mmol, 1.88 mL) in dioxane/water (50 mL) added potassium phosphate tribasic anhydrous (4.71 g, 22.18 mmol). Argon was bubbled through the reaction mixture for 5 minutes before Pd(dppf)₂Cl₂CH2Cl₂ (1.21 g, 1.48 mmol) was added and the resulting suspension was purged with argon gas for an additional 5 minutes. The reaction was stirred at 100° C. for 1 hour. The progress of the reaction was monitored by LC-MS and TLC. After completion, the reaction mixture was cooled to room temperature and filtered through a Celite® bed. To the filtrate, water was added and the organics were extracted with ethyl acetate. The combined organic layers were washed with saturated brine solution and dried over anhydrous sodium sulfate to afford methyl 4-(3-(cyclopropylcarbamoyl)-7-fluoro-4-(methylamino)quinolin-6-yl)-2-fluorobenzoate (139b, 4.4 g, crude) which is used for the next step without further purification. LCMS (ES+): m/z 412 [M+H]+

Step 6B (Synthesis of Compound 139c): To a solution of 4-(3-bicyclo[1.1.1]pentanylamino)-6-bromo-7-fluoro-N-methyl-quinoline-3-carboxamide (137c, 220 mg, 604.04 μmol) in 1,4 dioxane (6 mL) and water (2 mL) was added (3-fluoro-4-methoxycarbonyl-phenyl)boronic acid (138b, 143.49 mg, 724.85 μmol) and potassium phosphate tribasic (320.55 mg, 1.51 mmol). The resulting mixture was purged with nitrogen for 5 minutes and Pd(dppf)Cl₂ (44.20 mg, 60.40 μmol) was added. The reaction was stirred for 2 hours at 80° C. The reaction was then cooled to room temperature, diluted with water and extracted with ethyl acetate (3×20 mL). The combined organic extracts were dried over anhydrous sodium sulfate, filtered and concentrated under reduced pressure. The resulting crude material was purified by column chromatography on silica eluted with 5% methanol in dichloromethane to yield methyl 4-[4-(3-bicyclo[1.1.1]pentanylamino)-7-fluoro-3-(methylcarbamoyl)-6-quinolyl]-2-fluoro-benzoate (139c, 240 mg, 548.65 μmol, 90.83% yield) as a pale brown colored solid. LCMS (ES+): m/z 438 [M+H]+

Step 6B (Synthesis of Compound 139d): To a solution of 4-(3-bicyclo[1.1.1]pentanylamino)-6-bromo-7-fluoro-N-methyl-quinoline-3-carboxamide (137c, 120 mg, 329.48 μmol) and (6-methoxycarbonyl-3-pyridyl)boronic acid (138c, 71.54 mg, 395.37 μmol) in 1,4-dioxane (6 mL) and water (2 mL) was added potassium phosphate tribasic (174.85 mg, 823.70 μmol) and Pd(dppf)Cl₂ (24.11 mg, 32.95 μmol). The resulting mixture was stirred for 2 hours at 80° C. The reaction was then cooled to ambient temperature, diluted with water, and extracted with ethyl acetate (3×20 mL). The combined organic extracts were dried over anhydrous sodium sulfate, filtered and concentrated under reduced pressure. The resulting crude material was purified by column chromatography on silica eluted with 5% methanol in dichloromethane to yield methyl 5-[4-(3-bicyclo[1.1.1]pentanylamino)-7-fluoro-3-(methylcarbamoyl)-6-quinolyl]pyridine-2-carboxylate (139d, 120 mg, 185.52 μmol, 56.31% yield) as an pale brown colored solid. LCMS (ES+): m/z 421 [M+H]+

Step 7B (Synthesis of Compound 140a) Into a 50 mL single-necked round-bottomed flask containing a well-stirred solution of methyl 4-[4-anilino-3-(cyclopropylcarbamoyl)-7-fluoro-6-quinolyl]-2-fluoro-benzoate (139a, 0.6 g, 1.27 mmol) in methanol (6 mL) was added lithium hydroxide powder, reagent grade (151.75 mg, 6.34 mmol) in water (6 mL) at room temperature. The resulting mixture was stirred at room temperature for 16 hours. The reaction mixture was concentrated under reduced pressure and the pH of the aqueous solution was adjusted to pH=3 with saturated citric acid. The solution was stirred for 10 minutes and the resulting solid was filtered and dried to afford 550 mg of 4-[4-anilino-3-(cyclopropylcarbamoyl)-7-fluoro-6-quinolyl]-2-fluoro-benzoic acid (140a, 550 mg, 1.20 mmol, 94.47% yield) as a yellow solid. LCMS (ES+): m/z 460 [M+H]⁺

Step 7B (Synthesis of Compound 140b): Into a 100 mL single-necked round-bottomed flask containing a well-stirred solution of methyl 4-[3-(cyclopropylcarbamoyl)-7-fluoro-4-(methylamino)-6-quinolyl]-2-fluoro-benzoate (139b, 4.4 g, 10.70 mmol) in THF/H₂O (40/10 mL) was added MeOH (1.47 g, 10.70 mmol) and LiOH (7.06 g, 53.48 mmol) and the mixture was stirred at room temperature for 16 hours. The progress of the reaction was monitored by LC-MS and TLC and after completion, the reaction volatiles were removed under vacuum and the resulting solution was acidified with citric acid solution to obtain a solid precipitate that was filtered and dried to afford 4-[3-(cyclopropylcarbamoyl)-7-fluoro-4-(methylamino)-6-quinolyl]-2-fluoro-benzoic acid (140b, 4 g, 8.05 mmol, 75.29% yield) as a white color liquid. LCMS (ES+): m/z 398 [M+H]+

Step 7B (Synthesis of Compound 140c): To a solution of methyl 4-[4-(3-bicyclo[1.1.1]pentanylamino)-7-fluoro-3-(methylcarbamoyl)-6-quinolyl]-2-fluoro-benzoate (139c, 240 mg, 548.65 mol) in THF (10 mL) and methanol (1 mL) was added a solution of lithium hydroxide monohydrate (69.06 mg, 1.65 mmol, 45.74 uL) in water the reaction was stirred for 16 hours at 25° C. The reaction was partially concentrated under reduced pressure and the pH of the mixture was adjusted to approximately 3 with saturated citric acid solution. The resulting solid was filtered, washed with water, and dried under vacuum to yield 4-[4-(3-bicyclo[1.1.1]pentanylamino)-7-fluoro-3-(methylcarbamoyl)-6-quinolyl]-2-fluoro-benzoic acid (140c, 170 mg, 317.19 mol, 57.81% yield, 79% purity) as an off white solid. LCMS (ES+): m/z 424 [M+H]⁺

Step 7B (Synthesis of Compound 140d): To a solution of methyl 5-[4-(3-bicyclo[1.1.1]pentanylamino)-7-fluoro-3-(methylcarbamoyl)-6-quinolyl]pyridine-2-carboxylate (139d, 120 mg, 285.42 mol) in THF (10 mL) and methanol (1 mL) was added a solution of lithium hydroxide, monohydrate (35.93 mg, 856.26 mol, 23.79 uL) in water (2 mL) and the reaction was stirred for 16 hours at 25° C. The reaction was partially concentrated under reduced pressure and the pH of the mixture was adjusted to pH of approximately 3 with saturated citric acid solution. The resulting solid was filtered, washed with water, and dried under vacuum to yield 5-[4-(3-bicyclo[1.1.1]pentanylamino)-7-fluoro-3-(methylcarbamoyl)-6-quinolyl]pyridine-2-carboxylic acid (140d, 80 mg, 64.96 mol, 22.76% yield) as a pale brown solid. LCMS (ES+): m/z 407 [M+H]+

### MTH1 Degrader Synthesis

### Synthesis of Compound 300

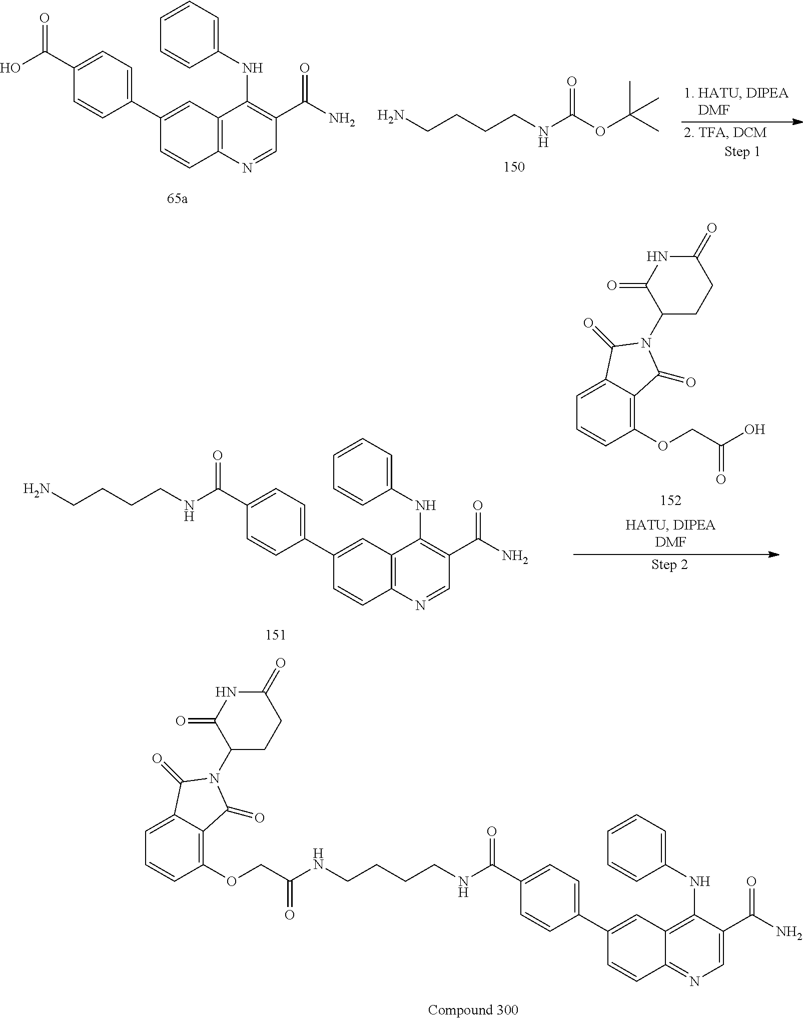 [SMILES: NC(=O)c1cnc2ccc(-c3ccc(C(=O)O)cc3)cc2c1Nc1ccccc1 ; CC(C)(C)OC(=O)NCCCCN ; NCCCCNC(=O)c1ccc(-c2ccc3ncc(C(N)=O)c(Nc4ccccc4)c3c2)cc1 ; O=C(O)COc1cccc2c1C(=O)N(C1CCC(=O)NC1=O)C2=O ; NC(=O)c1cnc2ccc(-c3ccc(C(=O)NCCCCNC(=O)COc4cccc5c4C(=O)N(C4CCC(=O)NC4=O)C5=O)cc3)cc2c1Nc1ccccc1]

Step 1.1: To an oven-dried round bottom flask charged with a solution of 4-(4-anilino-3-carbamoyl-6-quinolyl)benzoic acid (65a, 80 mg, 208.66 μmol) in DMF (3 mL), tert-butyl N-(4-aminobutyl)carbamate (150, 47.14 mg, 250.39 μmol), DIPEA (134.84 mg, 1.04 mmol, 181.72 uL) and HATU (95.21 mg, 250.39 μmol) were added. The reaction mixture was stirred for 16 hours at room temperature. The reaction mixture was quenched with water (5 mL) and the product was extracted with ethyl acetate (2×25 mL). The combined organic layer was dried over anhydrous sodium sulfate, filtered and concentrated under reduced pressure to yield tert-butyl N-[4-[[4-(4-anilino-3-carbamoyl-6-quinolyl) benzoyl]amino]butyl]carbamate (130 mg, 122.92 μmol, 58.91% yield) as brown gummy oil. LCMS (ES+): m/z 668 [M+H]+

Step 1.2: An oven dried pressure tube was charged with tert-butyl N-[4-[[4-(4-anilino-3-carbamoyl-6-quinolyl)benzoyl]amino]butyl]carbamate (120 mg, 216.74 μmol) in dichloromethane (2 mL) and trifluoroacetic acid (2.96 g, 25.96 mmol, 2 mL) was added at room temperature. The reaction mixture was stirred for an hour at room temperature. The reaction mixture was concentrated under reduced pressure. The crude mixture was purified by reverse phase column chromatography to yield 6-[4-(4-aminobutylcarbamoyl)phenyl]-4-anilino-quinoline-3-carboxamide (151, 75 mg, 163.63 μmol, 75.50% yield) as white solid. LCMS (ES+): m/z 454 [M+H]⁺

Step 2: To a stirred solution of 2-[2-(2,6-dioxo-3-piperidyl)-1,3-dioxo-isoindolin-4-yl]oxyacetic acid (152, 500 mg, 1.50 mmol), 6-[4-(4-aminobutylcarbamoyl)phenyl]-4-anilino-quinoline-3-carboxamide (151, 750.74 mg, 1.66 mmol) in DMF (20.0 mL) was added DIPEA (972.44 mg, 7.52 mmol, 1.31 mL) and HATU (858.27 mg, 2.26 mmol) and the reaction was stirred for 16 hr at 25° C. The reaction mixture was diluted with water (50 mL) and extracted with ethyl acetate (3×25 mL). The combined organic extracts were washed with water and brine solution, dried over anhydrous sodium sulfate, filtered and concentrated under reduced pressure. The resulting crude material was purified by reverse phase preparative HPLC to yield 4-anilino-6-[4-[4-[[2-[2-(2,6-dioxo-3-piperidyl)-1,3-dioxo-isoindolin-4-yl]oxyacetyl]amino]butylcarbamoyl]phenyl]quinoline-3-carboxamide (Compound 300, 450 mg, 575.32 μmol, 38.23% yield) as an yellow solid. ¹H NMR (400 MHz, DMSO-d6) δ 12.29 (s, 1H), 11.18-11.09 (m, 1H), 9.01 (s, 1H), 8.57 (t, J=5.7 Hz, 1H), 8.36 (d, J=8.6 Hz, 2H), 8.28 (s, 1H), 8.10-8.04 (m, 1H), 8.01 (t, J=5.7 Hz, 1H), 7.91 (d, J=7.3 Hz, 3H), 7.80 (t, J=7.7 Hz, 1H), 7.57-7.50 (m, 4H), 7.49-7.45 (m, 1H), 7.44-7.36 (m, 4H), 5.11 (dd, J=12.9, 5.6 Hz, 1H), 4.78 (s, 2H), 3.33-3.24 (m, 2H), 3.24-3.13 (m, 2H), 2.95-2.82 (m, 1H), 2.60 (s, 1H), 2.08-1.99 (m, 1H), 1.52 (s, 4H). LCMS (ES+): m/z 768 [M+H]+

### Synthesis of Compound 301

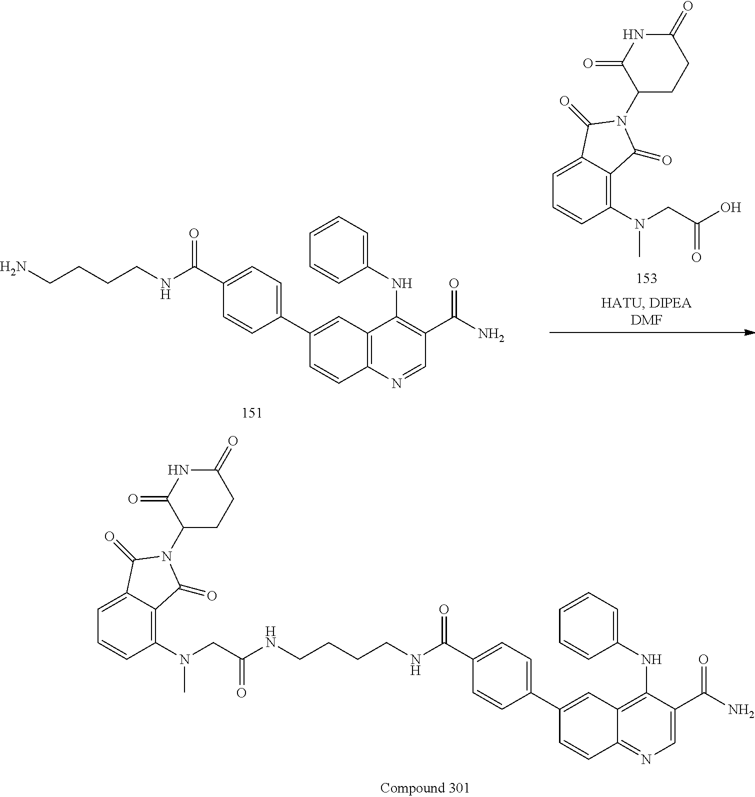 [SMILES: NCCCCNC(=O)c1ccc(-c2ccc3ncc(C(N)=O)c(Nc4ccccc4)c3c2)cc1 ; CN(CC(=O)O)c1cccc2c1C(=O)N(C1CCC(=O)NC1=O)C2=O ; CN(CC(=O)NCCCCNC(=O)c1ccc(-c2ccc3ncc(C(N)=O)c(Nc4ccccc4)c3c2)cc1)c1cccc2c1C(=O)N(C1CCC(=O)NC1=O)C2=O]

To a stirred solution of 2-[[2-(2,6-dioxo-3-piperidyl)-1,3-dioxo-isoindolin-4-yl]-methyl-amino]acetic acid (153, 50 mg, 144.80 μmol) in DMF (10 mL) was added 6-[4-(4-aminobutylcarbamoyl)phenyl]-4-anilino-quinoline-3-carboxamide (151, 65.67 mg, 144.80 μmol), N,N-diisopropylethylamine (93.57 mg, 724.00 mol, 126.10 uL) and HATU (82.59 mg, 217.20 mol) at room temperature and the reaction was stirred for 16 hours. Water (20 mL) was added and the organics were extracted with ethyl acetate (3×20 mL). The combined organic extracts were washed with water and brine solution, dried over anhydrous sodium sulfate, filtered and concentrated under reduced pressure. The resulting crude material was purified by reverse phase preparative HPLC to yield 4-anilino-6-[4-[4-[[2-[[2-(2,6-dioxo-3-piperidyl)-1,3-dioxo-isoindolin-4-yl]-methyl-amino]acetyl]amino]butylcarbamoyl]phenyl]quinoline-3-carboxamide (Compound 301, 45 mg, 55.59 mol, 38.39% yield) as an yellow solid. ¹H NMR (400 MHz, DMSO-d₆) δ 12.17 (s, 1H), 11.07 (s, 1H), 9.01 (s, 1H), 8.54 (t, J=5.6 Hz, 1H), 8.36-8.31 (m, 2H), 8.25 (s, 1H), 8.06 (d, J=8.8 Hz, 1H), 7.96 (t, J=5.7 Hz, 1H), 7.92-7.88 (m, 3H), 7.62 (dd, J=8.5, 7.0 Hz, 1H), 7.54-7.48 (m, 4H), 7.44-7.35 (m, 3H), 7.27-7.21 (m, 2H), 5.08 (dd, J=12.8, 5.4 Hz, 1H), 4.22-4.08 (m, 2H), 3.26 (q, J=6.4 Hz, 2H), 3.17 (s, 2H), 3.10 (q, J=6.4 Hz, 2H), 3.00 (s, 3H), 2.87 (ddd, J=17.2, 13.9, 5.4 Hz, 1H), 2.60-2.55 (m, 2H), 2.05-1.97 (m, 1H), 1.58-1.39 (m, 4H). LCMS (ES+): m/z 780 [M+H]+

### Synthesis of Compound 302

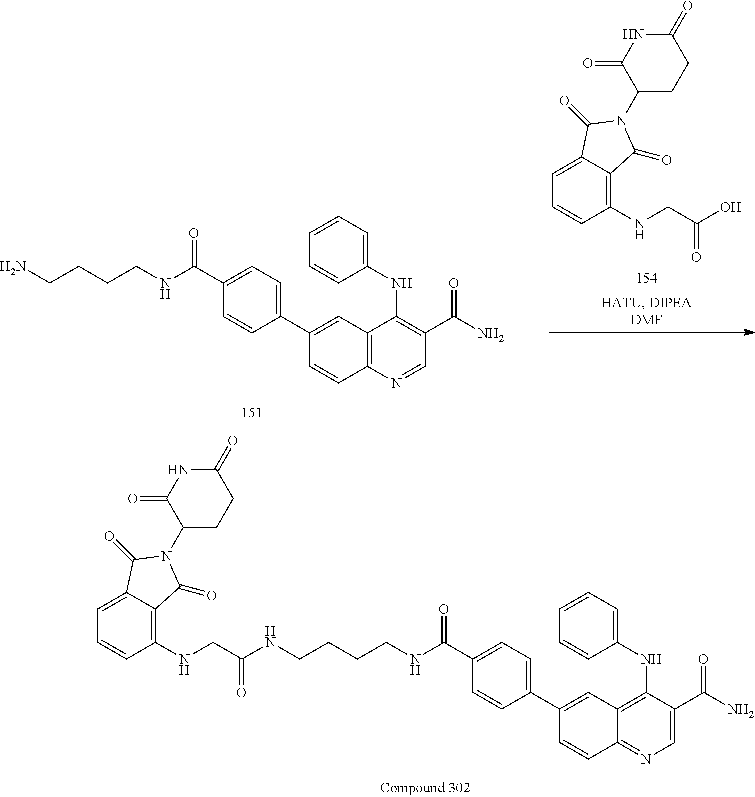 [SMILES: NCCCCNC(=O)c1ccc(-c2ccc3ncc(C(N)=O)c(Nc4ccccc4)c3c2)cc1 ; O=C(O)CNc1cccc2c1C(=O)N(C1CCC(=O)NC1=O)C2=O ; NC(=O)c1cnc2ccc(-c3ccc(C(=O)NCCCCNC(=O)CNc4cccc5c4C(=O)N(C4CCC(=O)NC4=O)C5=O)cc3)cc2c1Nc1ccccc1]

To a stirred solution of 2-((2-(2,6-dioxopiperidin-3-yl)-1,3-dioxoisoindolin-4-yl)amino)acetic acid (154, 40 mg, 0.1207 mmol) in DMF (10.0 mL) was added 6-(4-((4-aminobutyl)carbamoyl)phenyl)-4-(phenylamino)quinoline-3-carboxamide (151, 60.1 mg, 0.1327 mmol), HATU (69 mg, 0.181 mmol) and N,N-diisopropylethylamine (0.5 mL) at room temperature and the reaction was stirred overnight at room temperature. Water (25 ml) was added and the reaction was extracted with ethyl acetate (3×20 mL). The combined organic extracts were washed with water and brine solution, dried over anhydrous sodium sulfate, filtered and concentrated under reduced pressure. The resulting crude was purified by reverse phase preparative HPLC to yield 6-(4-((4-(2-((2-(2,6-dioxopiperidin-3-yl)-1,3-dioxoisoindolin-4 yl)amino)acetamido)butyl)carbamoyl)phenyl)-4-(phenylamino)quinoline-3-carboxamide (Compound 301, 22.7 mg, 0.02968 mmol, 10%) as a yellow colored solid. ¹H NMR (400 MHz, DMSO-d₆) δ 12.17 (s, 1H), 11.11 (s, 1H), 9.01 (d, J=1.9 Hz, 1H), 8.56 (t, J=5.7 Hz, 1H), 8.34 (d, J=8.0 Hz, 2H), 8.26 (s, 1H), 8.14 (t, J=5.8 Hz, 1H), 8.05 (dd, J=8.8, 2.0 Hz, 1H), 7.93-7.88 (m, 3H), 7.58 (ddd, J=8.6, 7.0, 1.5 Hz, 1H), 7.54-7.48 (m, 4H), 7.45-7.37 (m, 3H), 7.05-7.01 (m, 1H), 6.97-6.92 (m, 1H), 6.88-6.82 (m, 1H), 5.11-5.01 (m, 1H), 3.95-3.88 (m, 2H), 3.31-3.22 (m, 2H), 3.20-3.09 (m, 2H), 2.95-2.82 (m, 1H), 2.63-2.53 (m, 2H), 2.07-1.97 (m, 1H), 1.58-1.40 (m, 4H). LCMS (ES+): m/z 767 [M+H]+

### Synthesis of Compound 303

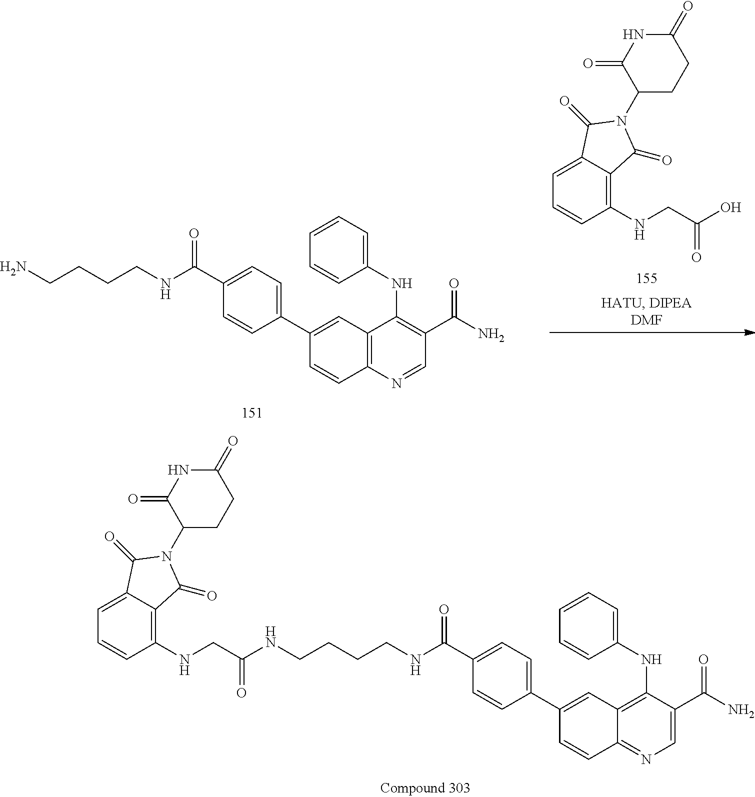 [SMILES: NCCCCNC(=O)c1ccc(-c2ccc3ncc(C(N)=O)c(Nc4ccccc4)c3c2)cc1 ; O=C(O)CNc1cccc2c1C(=O)N(C1CCC(=O)NC1=O)C2=O ; NC(=O)c1cnc2ccc(-c3ccc(C(=O)NCCCCNC(=O)CNc4cccc5c4C(=O)N(C4CCC(=O)NC4=O)C5=O)cc3)cc2c1Nc1ccccc1]

An oven dried round bottom flask was charged with a solution of 6-(4-((4-aminobutyl)carbamoyl)phenyl)-4-(phenylamino)quinoline-3-carboxamide (151, 57 mg, 126.07 μmol) in DMF (2 mL) and 2-[[2-(2,6-dioxo-3-piperidyl)-1-oxo-isoindolin-4-yl]amino]acetic acid (155, 40 mg, 126.07 μmol), DIPEA (81.46 mg, 630.33 μmol, 109.79 uL) and HATU (57.52 mg, 151.28 μmol) were added. The reaction mixture was stirred for 16 hours at room temperature and the reaction mixture was concentrated under reduced pressure. The crude mixture was purified by reverse phase column chromatography (Column: SUNFIRE OBD C18(100×30)MM 5μ), mobile phase: A: 0.1% TFA in water, B: ACN) to yield 4-anilino-6-[4-[4-[[2-[[2-(2,6-dioxo-3-piperidyl)-1-oxo-isoindolin-4-yl]amino]acetyl]amino]butylcarbamoyl]phenyl]quinoline-3-carboxamide (Compound 303, 26.87 mg, 33.38 μmol, 26.48% yield) as a yellow solid. ¹H NMR (400 MHz, DMSO-d₆) δ 12.11 (s, 1H), 11.02 (s, 1H), 9.00 (s, 1H), 8.54 (t, J=5.9 Hz, 1H), 8.38-8.29 (m, 2H), 8.26 (s, 1H), 8.04 (d, J=8.8 Hz, 1H), 8.00 (t, J=5.9 Hz, 1H), 7.90 (d, J=8.1 Hz, 3H), 7.51 (t, J=8.4 Hz, 3H), 7.45-7.35 (m, 2H), 7.26 (t, J=7.7 Hz, 1H), 6.95 (d, J=7.4 Hz, 1H), 6.55 (d, J=8.0 Hz, 1H), 6.08 (s, 1H), 5.12 (dd, J=13.3, 5.1 Hz, 1H), 4.28 (d, J=17.0 Hz, 1H), 4.18 (d, J=17.1 Hz, 1H), 3.73 (s, 2H), 3.24 (q, J=6.3 Hz, 2H), 3.11 (q, J=6.3 Hz, 2H), 2.98-2.86 (m, 2H), 2.64-2.57 (m, 1H), 2.06-1.97 (m, 1H), 1.53-1.36 (m, 3H). LCMS (ES+): m/z: 754 [M+H]⁺

### Synthesis of Compound 304

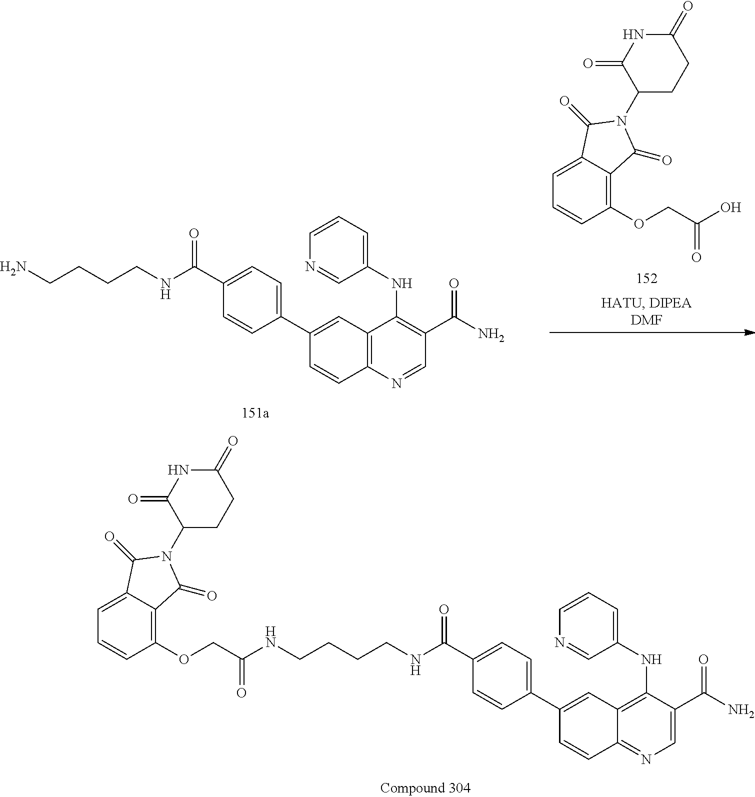 [SMILES: NCCCCNC(=O)c1ccc(-c2ccc3ncc(C(N)=O)c(Nc4cccnc4)c3c2)cc1 ; O=C(O)COc1cccc2c1C(=O)N(C1CCC(=O)NC1=O)C2=O ; NC(=O)c1cnc2ccc(-c3ccc(C(=O)NCCCCNC(=O)COc4cccc5c4C(=O)N(C4CCC(=O)NC4=O)C5=O)cc3)cc2c1Nc1cccnc1]

Into a 25 mL single-necked round-bottomed flask containing a well-stirred solution of 2-[2-(2,6-dioxo-3-piperidyl)-1,3-dioxo-isoindolin-4-yl]oxyacetic acid (152, 109.65 mg, 330.02 μmol) and 6-[4-(4-aminobutylcarbamoyl)phenyl]-4-(3-pyridylamino)quinoline-3-carboxamide (151a, 0.1 g, 220.01 μmol) in DMF (5 mL) were added DIPEA (85.30 mg, 660.03 μmol, 114.97 uL) and HATU (125.48 mg, 330.02 μmol) under nitrogen atmosphere at room temperature and stirred for 16 hrs. After completion of the reaction, 20 mL of cold water was added and stirred for 10 minutes. The resulting solid was filtered and dried to afford crude product, which was purified by reverse phase prep purification (SUNFIRE OBD C18(100×30)MM 5μ), mobile phase: A: 0.1% TFA in water B: ACN) to obtain 6-[4-[4-[[2-[2-(2,6-dioxo-3-piperidyl)-1,3-dioxo-isoindolin-4-yl]oxyacetyl]amino]butylcarbamoyl]phenyl]-4-(3-pyridylamino)quinoline-3-carboxamide (Compound 304, 40.0 mg, 52.03 μmol, 23.65% yield). ¹H NMR (400 MHz, DMSO-d₆) δ 11.43 (s, 1H), 11.12 (s, 1H), 8.95 (s, 1H), 8.68 (s, 1H), 8.62-8.56 (m, 2H), 8.55-8.51 (m, 1H), 8.46-8.40 (m, 1H), 8.18 (s, 1H), 8.12 (d, J=8.9 Hz, 1H), 8.03-7.96 (m, 3H), 7.84-7.77 (m, 4H), 7.71 (s, 1H), 7.54 (dd, J=8.2, 4.8 Hz, 1H), 7.48 (d, J=7.2 Hz, 1H), 7.39 (d, J=8.5 Hz, 1H), 5.11 (dd, J=12.9, 5.4 Hz, 1H), 4.78 (s, 2H), 3.34-3.25 (m, 2H), 3.19 (q, J=6.3 Hz, 2H), 2.95-2.82 (m, 1H), 2.61-2.53 (m, 2H), 2.08-1.98 (m, 1H), 1.60-1.47 (m, 4H). LCMS (ES+): m/z 769 [M+H]⁺

### Synthesis of Compound 305

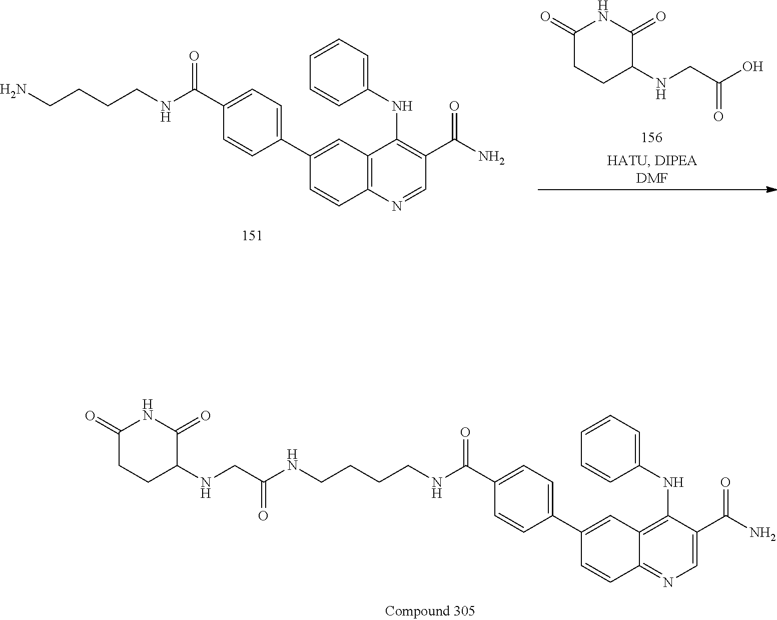 [SMILES: NCCCCNC(=O)c1ccc(-c2ccc3ncc(C(N)=O)c(Nc4ccccc4)c3c2)cc1 ; O=C(O)CNC1CCC(=O)NC1=O ; NC(=O)c1cnc2ccc(-c3ccc(C(=O)NCCCCNC(=O)CNC4CCC(=O)NC4=O)cc3)cc2c1Nc1ccccc1]

To a stirred solution of 2-[(2,6-dioxo-3-piperidyl)amino]aceticacid (156, 130 mg, 698.31 μmol) and 6-[4-(4-aminobutylcarbamoyl)phenyl]-4-anilino-quinoline-3-carboxamide (151, 316.71 mg, 698.31 μmol) in DMF (10.0 mL) was added DIPEA (451.26 mg, 3.49 mmol, 608.16 uL) and HATU (398.28 mg, 1.05 mmol). The resulting solution was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and the solvent evaporated under reduced pressure. The resulting crude was purified by reverse phase preparative HPLC to yield 4-anilino-6-[4-[4-[[2-[(2,6-dioxo-3-piperidyl)amino]acetyl]amino]butylcarbamoyl]phenyl]quinoline-3-carboxamide (Compound 305, 3.05 mg, 4.68 μmol, 0.67% yield) as an yellow solid. ¹H NMR (400 MHz, Methanol-d₄) δ 8.99 (s, 1H), 8.28 (dd, J=8.8, 1.9 Hz, 1H), 8.09 (d, J=1.9 Hz, 1H), 8.00 (d, J=8.8 Hz, 1H), 7.84 (d, J=8.5 Hz, 2H), 7.63-7.55 (m, 2H), 7.55-7.50 (m, 1H), 7.47-7.43 (m, 2H), 7.41-7.37 (m, 2H), 4.28 (dd, J=13.2, 5.1 Hz, 1H), 3.93 (d, J=3.3 Hz, 2H), 3.42 (t, J=6.6 Hz, 2H), 3.38-3.33 (m, 2H), 2.81-2.73 (m, 2H), 2.42-2.32 (m, 1H), 2.10 (qd, J=12.5, 6.7 Hz, 1H), 1.75-1.59 (m, 4H). LCMS (ES+): m/z 622 [M+H]⁺

### Synthesis of Compound 306

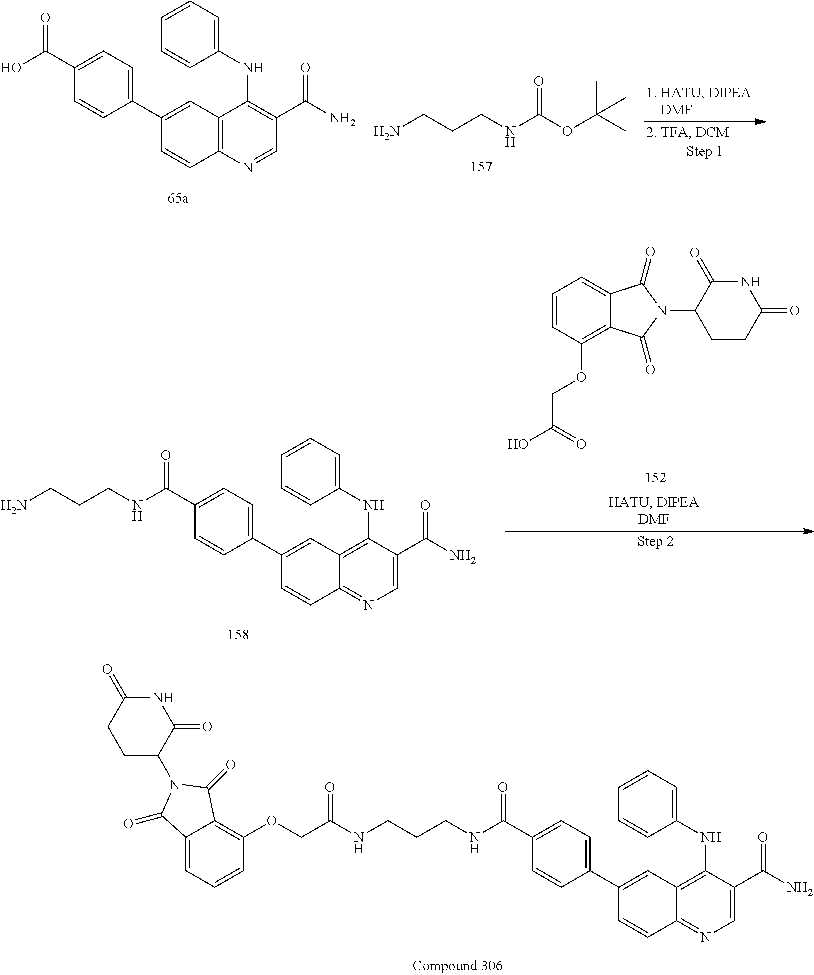 [SMILES: NC(=O)c1cnc2ccc(-c3ccc(C(=O)O)cc3)cc2c1Nc1ccccc1 ; CC(C)(C)OC(=O)NCCCN ; NCCCNC(=O)c1ccc(-c2ccc3ncc(C(N)=O)c(Nc4ccccc4)c3c2)cc1 ; O=C(O)COc1cccc2c1C(=O)N(C1CCC(=O)NC1=O)C2=O ; NC(=O)c1cnc2ccc(-c3ccc(C(=O)NCCCNC(=O)COc4cccc5c4C(=O)N(C4CCC(=O)NC4=O)C5=O)cc3)cc2c1Nc1ccccc1]

Step 1.1: HATU mediated coupling of 4-(3-carbamoyl-4-(phenylamino)quinolin-6-yl)benzoic acid (65) and tert-butyl (3-aminopropyl)carbamate (57) in DMF was performed using the general procedure described in Step 1.1 of Synthesis of Compound 300.

Step 1.2: To a stirred solution of tert-butyl (3-(4-(3-carbamoyl-4-(phenylamino)quinolin-6-yl)benzamido)propyl)carbamate (50 mg, 0.09265 mmol) in Dichloromethane (10.0 mL) was added Trifluoroacetic acid (1.0 mL) and stirred for 30 minutes at room temperature. The resulting reaction mixture was concentrated completely under reduced pressure to yield 6-(4-((3-aminopropyl)carbamoyl)phenyl)-4-(phenylamino)quinoline-3-carboxamide (158, 47.2 mg, 0.1074 mmol) as a yellow colored solid. The resulting crude product was directly used for next step. LCM4S (ES+): m/z 440 [M+H]⁺

Step 2: To a stirred solution of 2-((2-(2,6-dioxopiperidin-3-yl)-1,3-dioxoisoindolin-4-yl)oxy)acetic acid (152, 50 mg, 0.1504 mmol) in DMF (5.00 mL) was added 6-(4-((3-aminopropyl)carbamoyl)phenyl)-4-(phenylamino)quinoline-3-carboxamide (158, 66.1 mg, 0.1504 mmol), HATU (85.7 mg, 0.2256 mmol) and N,N-Diisopropylethylamine (97.1 mg, 0.7519 mmol) at room temperature and stirred for over night at room temperature. Added water (25 ml) to the reaction mixture and extracted with ethyl acetate (3×20 mL). The combined organic extracts were washed with water and brine solution, dried over anhydrous sodium sulphate, filtered and evaporated the solvent completely under reduced pressure. The resulting crude was purified by reverse phase preparative HPLC to yield 6-(4-((3-(2-((2-(2,6-dioxopiperidin-3-yl)-1,3-dioxoisoindolin-4-yl)oxy)acetamido)propyl)carbamoyl)phenyl)-4-(phenylamino)quinoline-3-carboxamide (Compound 306, 38.2 mg, 0.050 mmol, 33.8%) as a yellow colored solid. ¹H NMR (400 MHz, DMSO-d₆) δ 12.11 (s, 1H), 11.12 (s, 1H), 9.01 (s, 1H), 8.55 (t, J=5.7 Hz, 1H), 8.36-8.29 (m, 2H), 8.23 (s, 1H), 8.07-8.01 (m, 2H), 7.89 (d, J=8.3 Hz, 3H), 7.85-7.78 (m, 1H), 7.53-7.46 (m, 6H), 7.43-7.35 (m, 5H), 5.12 (dd, J=12.9, 5.4 Hz, 1H), 4.79 (s, 2H), 3.30 (q, J=6.5 Hz, 2H), 3.22 (q, J=6.7 Hz, 2H), 2.89 (ddd, J=17.4, 13.9, 5.5 Hz, 1H), 2.63-2.53 (m, 2H), 2.07-1.97 (m, 1H), 1.71 (p, J=7.0 Hz, 2H). LCM4S (ES+): m/z 754 [M+H]⁺

### Synthesis of Compound 307

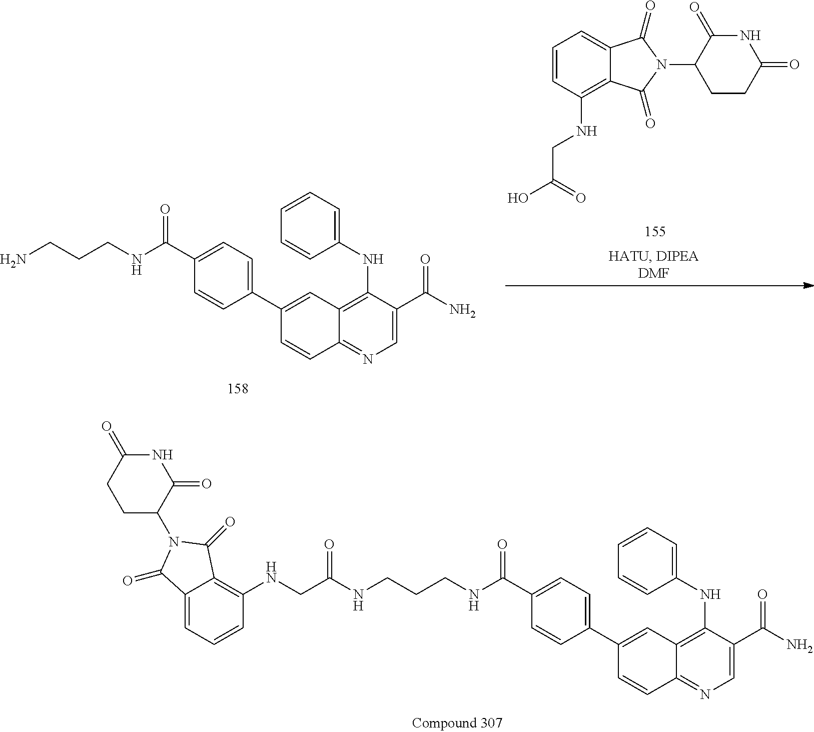 [SMILES: NCCCNC(=O)c1ccc(-c2ccc3ncc(C(N)=O)c(Nc4ccccc4)c3c2)cc1 ; O=C(O)CNc1cccc2c1C(=O)N(C1CCC(=O)NC1=O)C2=O ; NC(=O)c1cnc2ccc(-c3ccc(C(=O)NCCCNC(=O)CNc4cccc5c4C(=O)N(C4CCC(=O)NC4=O)C5=O)cc3)cc2c1Nc1ccccc1]

To a stirred solution of 2-((2-(2,6-dioxopiperidin-3-yl)-1,3-dioxoisoindolin-4-yl)amino)acetic acid (155, 40 mg, 0.1207 mmol) in DMF (5.00 mL) was added 6-(4-((3-aminopropyl)carbamoyl)phenyl)-4-(phenylamino)quinoline-3-carboxamide (158, 58.3 mg, 0.1327 mmol), HATU (69 mg, 0.181 m mol) and N,N-Diisopropylethylamine (0.5 mL) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by reverse phase preparative HPLC to yield 6-(4-((3-(2-((2-(2,6-dioxopiperidin-3-yl)-1,3-dioxoisoindolin-4-yl)amino)acetamido)propyl)carbamoyl)phenyl)-4-(phenylamino)quinoline-3-carboxamide (Compound 307, 25.7 mg, 0.03422 mmol, 28%) as a yellow colored solid. ¹H NMR (400 MHz, DMSO-d₆) δ 12.22 (s, 1H), 11.11 (s, 1H), 9.02 (s, 1H), 8.54 (t, J=5.6 Hz, 1H), 8.37-8.32 (m, 2H), 8.25 (s, 1H), 8.16 (t, J=5.7 Hz, 1H), 8.06 (d, J=8.8 Hz, 1H), 7.92-7.87 (m, 3H), 7.60 (t, J=7.8 Hz, 1H), 7.55-7.49 (m, 4H), 7.45-7.37 (m, 3H), 7.07 (d, J=7.1 Hz, 1H), 7.00-6.94 (m, 1H), 6.88 (d, J=8.5 Hz, 1H), 5.07 (dd, J=12.8, 5.4 Hz, 1H), 3.94 (d, J=5.0 Hz, 2H), 3.28 (q, J=6.6 Hz, 2H), 3.17 (q, J=6.8 Hz, 2H), 2.89 (ddd, J=16.9, 13.9, 5.4 Hz, 1H), 2.62-2.54 (m, 2H), 2.06-1.97 (m, 1H), 1.68 (p, J=7.0 Hz, 2H). LCMS (ES+): m/z 753 [M+H]⁺

### Synthesis of Compound 308

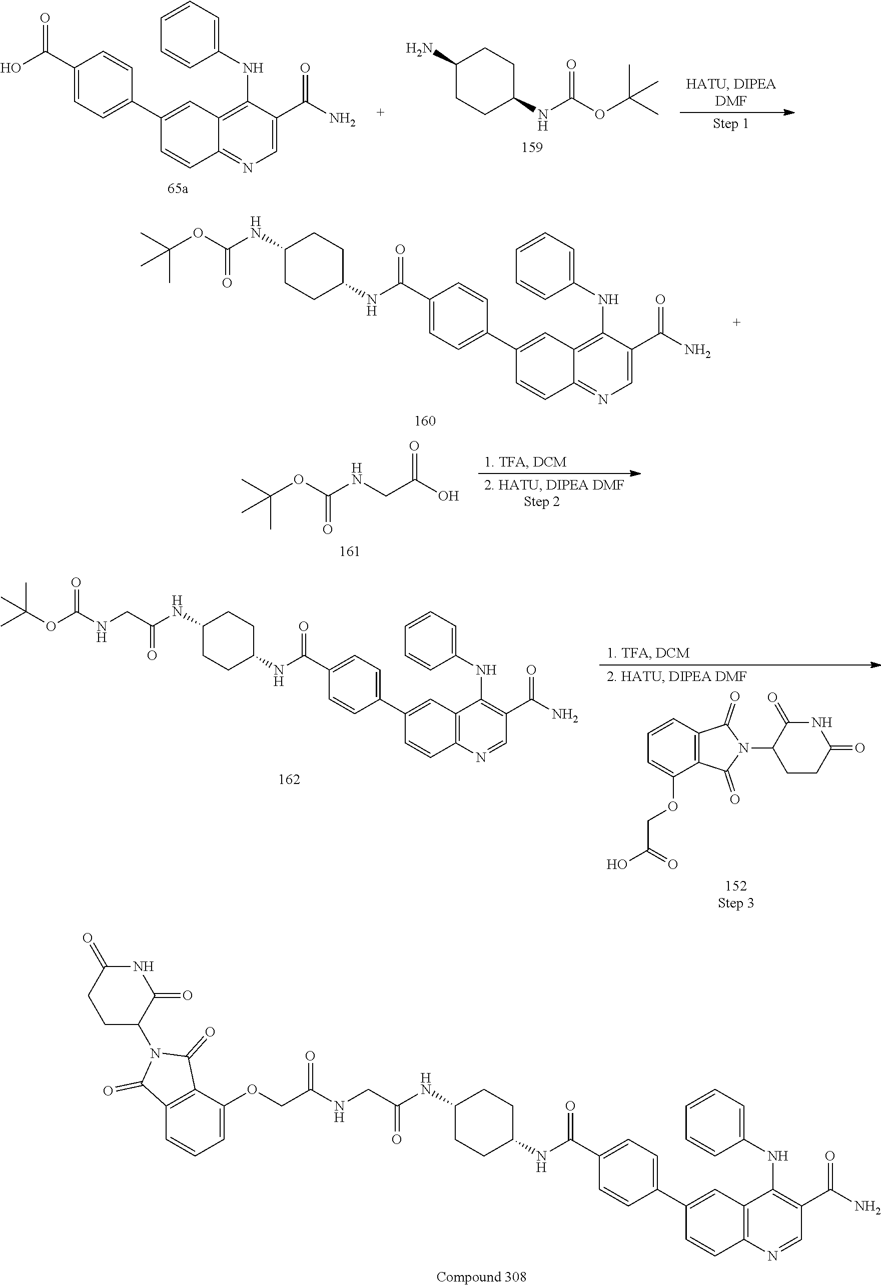

Step 1: Into a 25 mL single-necked round-bottomed flask containing a well-stirred solution of 4-(3-carbamoyl-4-(phenylamino)quinolin-6-yl)benzoic acid (65a, 60 mg, 0.1564 mmol) and tert-butyl ((1s,4s)-4-aminocyclohexyl)carbamate (159, 40.2 mg, 0.1876 mmol) in anhydrous DMF (3 mL) were added N-ethyl-N-isopropylpropan-2-amine (673 mg, 0.3128 mmol) and HATU (89 mg 0.2346 mmol) under nitrogen atmosphere at room temperature. The resulting mixture was stirred at room temperature for 16 h. 20 mL of ice cold water was added and stirred for 10 minutes, the resulting solid was filtered and dried to afford a crude solid which was purified by silica-gel (230-400 mesh) with 1:9 MeOH/DCM to generate tert-butyl ((1s,4s)-4-(4-(3-carbamoyl-4-(phenylamino)quinolin-6-yl)benzamido)cyclohexyl)carbamate (160, 60.0 mg, 0.1035 mmol, 66.2%) as a yellow solid. LCMS (ES+): m/z 580 [M+H]⁺

Step 2: Into a 25 mL single-necked round-bottomed flask containing a well-stirred solution of tert-butyl ((1s,4s)-4-(4-(3-carbamoyl-4-(phenylamino)quinolin-6-yl)benzamido)cyclohexyl)carbamate (160, 55 mg, 0.09487 mmol) in DCM (3 mL) was added 2,2,2-trifluoroacetic acid (1.5 mL, 0.09487 mmol) under nitrogen atmosphere at room temperature. The resulting mixture was stirred at room temperature for 30 minutes. The reaction mixture was concentrated under reduced pressure to afford a crude residue which was dissolved in anhydrous DMF (2 mL) were added 2-((tert-butoxycarbonyl)amino)acetic acid (161, 19.9 mg, 0.1138 mmol), N-ethyl-N-isopropylpropan-2-amine (24.5 mg, 0.1897 mmol) and HATU (54 mg 0.142 mmol) under nitrogen atmosphere at room temperature. The resulting mixture was stirred at room temperature for 16 h. 20 mL of Ice cold water was added and stirred for 10 minutes. The resulting solid was filtered and dried to afford 50 mg of tert-butyl (2-(((1s,4s)-4-(4-(3-carbamoyl-4-(phenylamino)quinolin-6-yl)benzamido)cyclohexyl)amino)-2-oxoethyl)carbamate (162, 50.0 mg, 0.07852 mmol, 82.7%) as an off white solid. LCMS (ES+): m/z 637 [M+H]⁺

Step 3: Into a 25 mL single-necked round-bottomed flask containing a well-stirred solution of tert-butyl (2-(((1 s,4s)-4-(4-(3-carbamoyl-4-(phenylamino)quinolin-6-yl)benzamido)cyclohexyl)amino)-2-oxoethyl)carbamate (162, 55 mg, 86.38 μmol) in anhydrous DCM (4 mL) was added TFA (49.25 mg, 431.89 μmol, 33.27 uL) under nitrogen atmosphere at room temperature. The resulting mixture was stirred at room temperature for 30 minutes. The reaction mixture was concentrated under reduced pressure to afford a crude residue which was dissolved in DMF (3 mL). DIPEA (33.49 mg, 259.13 μmol, 45.14 uL), 2-[2-(2,6-dioxo-3-piperidyl)-1,3-dioxo-isoindolin-4-yl]oxyacetic acid (152, 28.70 mg, 86.38 μmol) and HATU (49.27 mg, 129.57 μmol) were added under nitrogen atmosphere at room temperature. The resulting mixture was stirred at room temperature for 16 h. Ice cold water (15 mL) was added and stirred for 10 minutes, the resulting solid was filtered and dried to afford a crude solid which was purified by prep-HPLC(SUNFIRE OBD C18(100×30)MM 5μ) Mobile phase: A: 0.1% TFA in water B: ACN to afford 6-(4-(((1s,4s)-4-(2-(2-((2-(2,6-dioxopiperidin-3-yl)-1,3-dioxoisoindolin-4-yl)oxy)acetamido)acetamido)cyclohexyl)carbamoyl)phenyl)-4-(phenylamino)quinoline-3-carboxamide (Compound 308, 28 mg, 32.91 μmol, 38.10% yield, 061) as a pale yellow solid. ¹H NMR (400 MHz, DMSO-d₆) δ 11.12 (s, 1H), 9.01 (s, 1H), 8.38-8.30 (m, 2H), 8.28-8.20 (m, 2H), 8.20-8.14 (m, 1H), 8.05 (d, J=8.7 Hz, 1H), 7.91 (d, J=7.9 Hz, 3H), 7.82 (dd, J=10.2, 6.6 Hz, 2H), 7.50 (d, J=7.9 Hz, 5H), 7.44 (d, J=8.8 Hz, 2H), 7.42-7.35 (m, 2H), 5.12 (dd, J=12.9, 5.4 Hz, 1H), 4.86 (s, 2H), 3.84 (d, J=5.5 Hz, 3H), 3.73 (s, 2H), 2.96-2.82 (m, 2H), 2.08-1.99 (m, 1H), 1.80-1.69 (m, 4H), 1.67-1.51 (m, 5H). LCMS (ES+): m/z 851 [M+H]⁺

### Synthesis of Compound 309

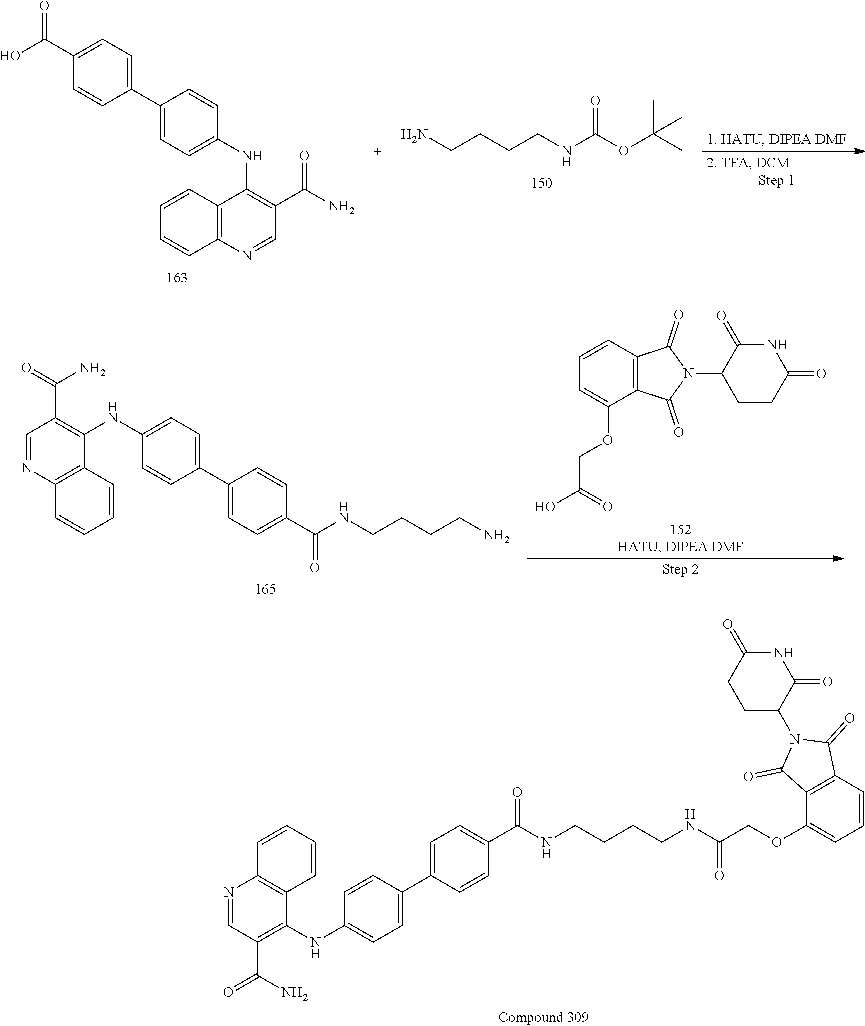 [SMILES: NC(=O)c1cnc2ccccc2c1Nc1ccc(-c2ccc(C(=O)O)cc2)cc1 ; CC(C)(C)OC(=O)NCCCCN ; NCCCCNC(=O)c1ccc(-c2ccc(Nc3c(C(N)=O)cnc4ccccc34)cc2)cc1 ; O=C(O)COc1cccc2c1C(=O)N(C1CCC(=O)NC1=O)C2=O ; NC(=O)c1cnc2ccccc2c1Nc1ccc(-c2ccc(C(=O)NCCCCNC(=O)COc3cccc4c3C(=O)N(C3CCC(=O)NC3=O)C4=O)cc2)cc1]

Step 1: To a stirred solution of 4-[4-[(3-carbamoyl-4-quinolyl)amino]phenyl]benzoic acid (163, 150 mg, 391.24 μmol) and tert-butyl N-(4-aminobutyl)carbamate (150, 110.49 mg, 586.86 μmol) in DMF (10 mL) was added DIPEA (252.82 mg, 1.96 mmol, 340.73 uL) and HATU (223.14 mg, 586.86 μmol). The mixture was stirred at for 16 hr at 25° C. The reaction mixture was diluted with water (10 mL) and extracted with ethyl acetate (3×25 mL). The combined organic extracts were washed with water and brine solution, dried over anhydrous sodium sulfate, filtered and evaporated the solvent completely under reduced pressure. The resulting crude tert-butyl N-[4-[[4-[4-[(3-carbamoyl-4-quinolyl)amino]phenyl]benzoyl]amino]butyl]carbamate (150 mg, 124.63 μmol, 31.85% yield, 46% purity) was taken as such for next step.

To a stirred solution of crude tert-butyl N-[4-[[4-[4-[(3-carbamoyl-4-quinolyl)amino]phenyl]benzoyl]amino]butyl]carbamate (150 mg, 270.93 μmol) in Dichloromethane (5 mL) was added TFA (2.96 g, 25.96 mmol, 2 mL) and stirred for 1 hr at 25° C. The reaction mixture was concentrated completely under reduced pressure to yield 4-[4-[4-(4-aminobutylcarbamoyl)phenyl]anilino]quinoline-3-carboxamide (165, 100 mg, 220.49 μmol, 81.38% yield) as a yellow solid. LCMS (ES+): m/z 454 [M+H]⁺

Step 2: To a stirred solution of 4-[4-[4-(4-aminobutylcarbamoyl)phenyl]anilino]quinoline-3-carboxamide (165, 50 mg, 110.25 μmol) and 2-[2-(2,6-dioxo-3-piperidyl)-1,3-dioxo-isoindolin-4-yl]oxyacetic acid (152, 54.95 mg, 165.38 μmol) in DMF (5 mL) was added DIPEA (71.24 mg, 551.25 μmol, 96.01 uL) and HATU (62.88 mg, 165.38 μmol) then stirred for 16 hr at 25° C. The reaction mixture was diluted with water (50 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-[4-[4-[4-[[2-[2-(2,6-dioxo-3-piperidyl)-1,3-dioxo-isoindolin-4-yl]oxyacetyl]amino]butylcarbamoyl]phenyl]anilino]quinoline-3-carboxamide (Compound 309, 15 mg, 17.97 μmol, 16.30% yield) as a yellow solid. LCMS (ES+): m/z 768 [M+H]⁺

### Synthesis of Compound 310

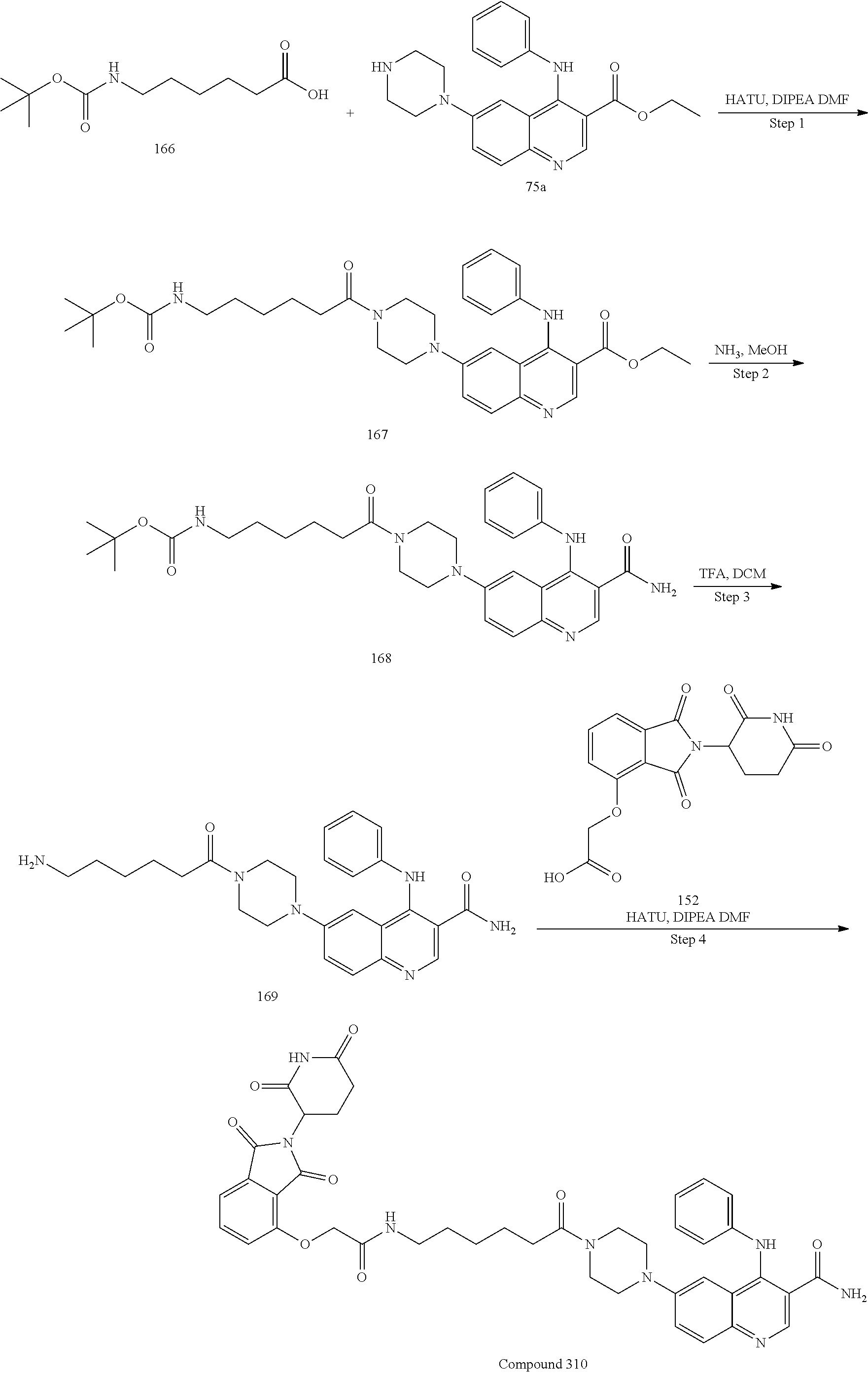

Step 1: To a stirred solution of 6-((tert-butoxycarbonyl)amino)hexanoic acid (166, 125 mg, 0.5404 mmol) in DMF (10.0 mL) was added ethyl 4-(phenylamino)-6-(piperazin-1-yl)quinoline-3-carboxylate (75a, 223 mg, 0.5944 mmol), DIPEA (1.0 mL) and HATU (309 mg, 0.8106 mmol) at rt. The resulting solution was stirred for 16 h at rt. Added water (25 mL) and extracted the reaction mixture with ethyl acetate (3×25 mL). The combined organic extracts were washed with water and brine solution, dried over anhydrous sodium sulfate, filtered and evaporated the solvent completely under reduced pressure. The resulting crude was purified by column chromatography on silica eluted with 5% methanol in dichloromethane to yield ethyl 6-(4-(6-((tert-butoxycarbonyl)amino)hexanoyl)piperazin-1-yl)-4-(phenylamino)quinoline-3-carboxylate (167, 168 mg, 0.2850 mmol, 52.8%) as a off white solid. LCMS (ES+): m/z 590 [M+H]⁺

Step 2: A stirred solution of ethyl 6-(4-(6-((tert-butoxycarbonyl)amino)hexanoyl)piperazin-1-yl)-4-(phenylamino)quinoline-3-carboxylate (167, 140 mg, 0.2373 mmol) in methanol (15 mL), was purged with ammonia gas for 5 minutes at −30° C. The resulting solution was heated to 80° C. and stirred for 24 h at 80° C. Evaporated the reaction mixture solvent completely under reduced pressure. The resulting crude was purified by column chromatography on silica eluted with 10% methanol in dichloromethane to yield tert-butyl (6-(4-(3-carbamoyl-4-(phenylamino)quinolin-6-yl)piperazin-1-yl)-6-oxohexyl)carbamate (168, 126 mg, 0.2260 mmol, 94.7%) as a yellow colored solid. LCMS (ES+): m/z 561 [M+H]⁺

Step 3: To a stirred solution of tert-butyl (6-(4-(3-carbamoyl-4-(phenylamino)quinolin-6-yl)piperazin-1-yl)-6-oxohexyl)carbamate (168, 140 mg, 0.2496 mmol) in Dichloromethane (10.0 mL) was added Trifluoroacetic acid (2.0 mL) and stirred for 30 minutes at room temperature. The resulting reaction mixture was concentrated completely under reduced pressure to yield 6-(4-(6-aminohexanoyl)piperazin-1-yl)-4-(phenylamino)quinoline-3-carboxamide (169, 140 mg, 0.3056 mmol, 100%) as a yellow colored solid.

Step 4: To a stirred solution of 2-((2-(2,6-dioxopiperidin-3-yl)-1,3-dioxoisoindolin-4-yl)oxy)acetic acid (152, 50 mg, 0.1504 mmol) in DMF (10.00 mL) was added 6-(4-(6-aminohexanoyl)piperazin-1-yl)-4-(phenylamino)quinoline-3-carboxamide (169, 76.1 mg, 0.1654 mmol), HATU (86 mg, 0.225 mmol) and N,N-Diisopropylethylamine (0.5 mL) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 6-(4-(6-(2-((2-(2,6-dioxopiperidin-3-yl)-1,3-dioxoisoindolin-4-yl)oxy)acetamido)hexanoyl)piperazin-1-yl)-4-(phenylamino)quinoline-3-carboxamide (Compound 310, 24.0 mg, 0.03100 mmol, 20.6%) as a yellow colored solid. ¹H NMR (400 MHz, DMSO-d₆) δ 11.78 (s, 1H), 11.12 (s, 1H), 8.80 (s, 1H), 8.24 (s, 1H), 7.95 (t, J=5.7 Hz, 1H), 7.86-7.77 (m, 4H), 7.52-7.43 (m, 3H), 7.39 (d, J=8.5 Hz, 1H), 7.36-7.28 (m, 3H), 7.14 (s, 1H), 5.12 (dd, J=12.8, 5.5 Hz, 1H), 4.76 (s, 2H), 3.51 (s, 3H), 3.19-3.08 (m, 5H), 2.98 (s, 2H), 2.94-2.83 (m, 1H), 2.63-2.54 (m, 2H), 2.36-2.28 (m, 2H), 2.07-1.99 (m, 1H), 1.56-1.39 (m, 4H), 1.34-1.23 (m, 2H). LCMS (ES+): m/z 775 [M+H]⁺

### Synthesis of Compound 311

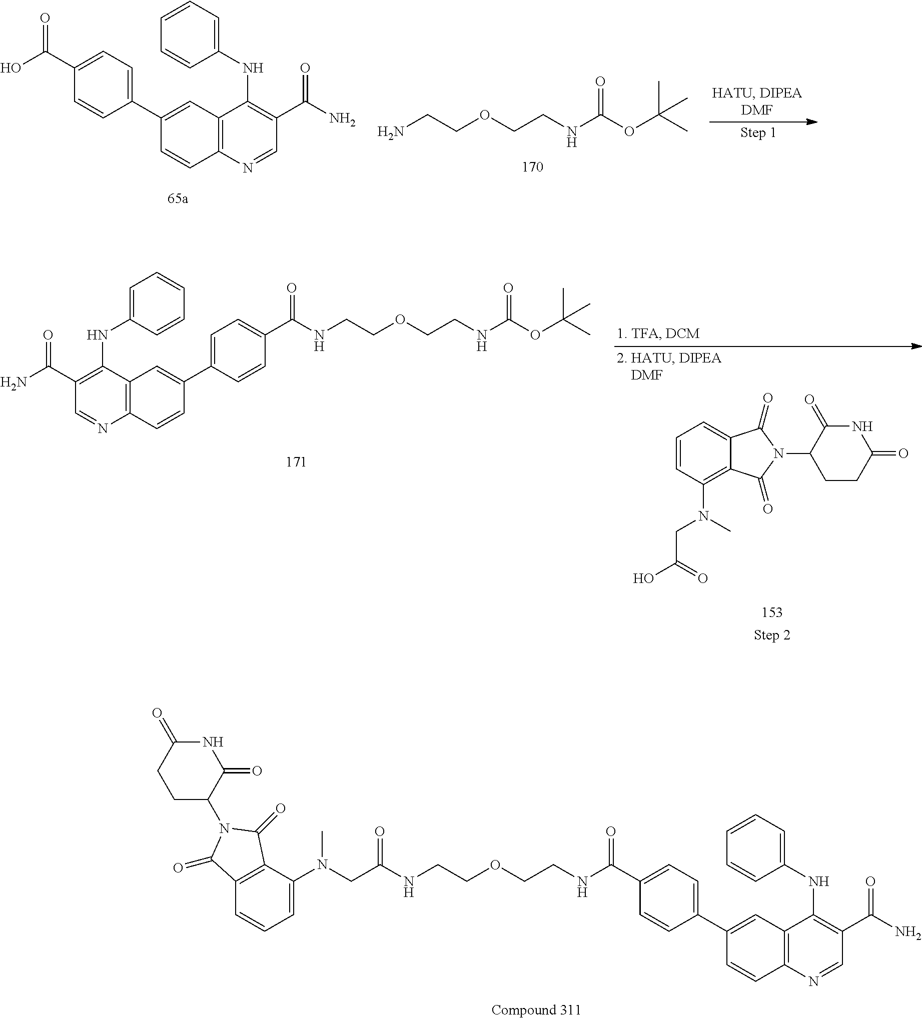 [SMILES: NC(=O)c1cnc2ccc(-c3ccc(C(=O)O)cc3)cc2c1Nc1ccccc1 ; CC(C)(C)OC(=O)NCCOCCN ; CC(C)(C)OC(=O)NCCOCCNC(=O)c1ccc(-c2ccc3ncc(C(N)=O)c(Nc4ccccc4)c3c2)cc1 ; CN(CC(=O)O)c1cccc2c1C(=O)N(C1CCC(=O)NC1=O)C2=O ; CN(CC(=O)NCCOCCNC(=O)c1ccc(-c2ccc3ncc(C(N)=O)c(Nc4ccccc4)c3c2)cc1)c1cccc2c1C(=O)N(C1CCC(=O)NC1=O)C2=O]

Step 1: To a solution of 4-(4-anilino-3-carbamoyl-6-quinolyl)benzoic acid (65a, 240 mg, 625.98 μmol) and tert-butyl N-[2-(2-aminoethoxy)ethyl]carbamate (170, 191.80 mg, 938.97 μmol) in N,N-dimethyl formamide (10 mL) were added N,N-Diisopropylethylamine (404.51 mg, 3.13 mmol, 545.16 uL) followed by HATU (476.03 mg, 1.25 mmol) under nitrogen atmosphere at room temperature. The resulting mixture was stirred at room temperature for 16 h. Ice cold water (15 mL) was added and stirred for 10 minutes, and then extracted with ethyl acetate. The combined organics were dried over anhydrous sodium sulfate and then concentrated to yield the crude product tert-butyl N-[2-[2-[[4-(4-anilino-3-carbamoyl-6-quinolyl)benzoyl]amino]ethoxy]ethyl]carbamate (171, 310 mg, 544.19 μmol, 83.46% yield). LCMS (ES+): m/z 570 [M+H]⁺

Step 2: To a stirred solution of crude tert-butyl N-[2-[2-[[4-(4-anilino-3-carbamoyl-6-quinolyl)benzoyl]amino]ethoxy]ethyl]carbamate (171, 100 mg, 175.55 μmol) in anhydrous Dichloromethane (5 mL) was added Trifluoroacetic acid (2.96 g, 25.96 mmol, 2.0 mL) at 0° C. The reaction mixture was stirred at room temperature for 3 hours. Then reaction mixture was concentrated completely and this crude was taken as such for the next step. To the stirred solution of above crude in N,N-Dimethylformamide (6 mL) was added 2-[[2-(2,6-dioxo-3-piperidyl)-1,3-dioxo-isoindolin-4-yl]-methyl-amino]acetic acid (153, 90.93 mg, 263.32 μmol) followed by HATU (100.12 mg, 263.32 μmol) and N,N-Diisopropylethylamine (113.44 mg, 877.73 μmol, 152.88 uL). The reaction mixture was stirred at room temperature for 16 hours. Ice cold water (15 mL) was added and stirred for 10 minutes, and then extracted with ethyl acetate. Then combined organics were dried over anhydrous sodium sulfate and then concentrated to yield the crude product, which was purified by prep HPLC (SUNFIRE OBD C18(100×30)MM 50 Mobile phase: A: 0.1% TFA in water B: ACN) to yield the product 4-anilino-6-[4-[2-[2-[[2-[[2-(2,6-dioxo-3-piperidyl)-1,3-dioxo-isoindolin-4-yl]-methyl-amino]acetyl]amino]ethoxy]ethylcarbamoyl]phenyl]quinoline-3-carboxamide (Compound 311, 30 mg, 33.88 μmol, 19.30% yield). ¹H NMR (400 MHz, DMSO-d₆) δ 11.63 (s, 1H), 11.07 (s, 1H), 9.01 (s, 1H), 8.56 (t, J=5.5 Hz, 1H), 8.33 (s, 1H), 8.22 (d, J=8.8 Hz, 1H), 8.15 (s, 1H), 8.03 (d, J=8.8 Hz, 1H), 7.99 (t, J=5.6 Hz, 1H), 7.89 (d, J=8.1 Hz, 2H), 7.82 (s, 1H), 7.64-7.55 (m, 1H), 7.51-7.41 (m, 4H), 7.34-7.18 (m, 5H), 5.07 (dd, J=12.9, 5.4 Hz, 1H), 4.17 (d, J=17.0 Hz, 1H), 4.11 (d, J=17.0 Hz, 1H), 3.54 (t, J=6.0 Hz, 2H), 3.48-3.35 (m, 4H), 3.25 (q, J=5.8 Hz, 2H), 2.96 (s, 3H), 2.87 (ddd, J=17.9, 14.0, 5.3 Hz, 1H), 2.60-2.53 (m, 2H), 2.04-1.95 (m, 1H). LCMS (ES+): m/z 798 [M+H]⁺

### Synthesis of Compound 312

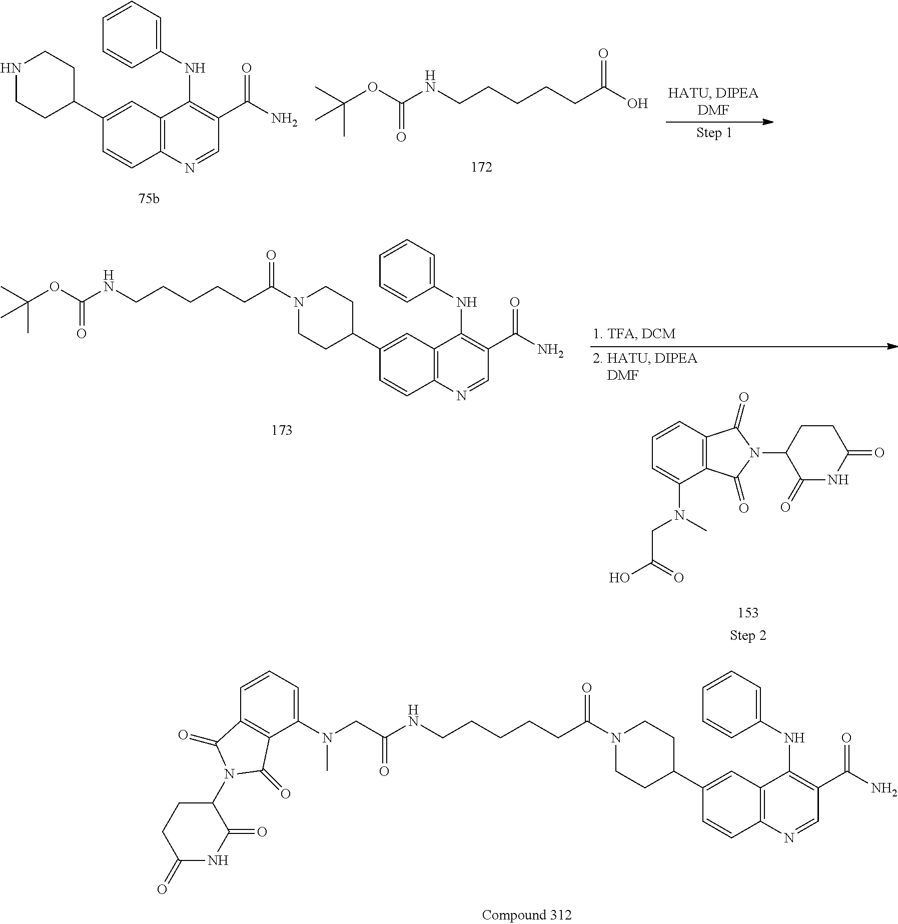

Step 1: To a stirred solution of 4-anilino-6-(4-piperidyl)quinoline-3-carboxamide (75b, 124 mg, 357.94 μmol) and 6-(tert-butoxycarbonylamino)hexanoic acid (172, 91.07 mg, 393.74 μmol) in DMF (10.0 mL) was added DIPEA (231.31 mg, 1.79 mmol, 311.74 uL) and HATU (204.15 mg, 536.91 μmol). The resulting solution was stirred for 16 hr at 25° C. The resulting mixture was diluted with water (20 mL) and extracted with ethyl acetate (3×25 mL). The combined organic extracts were washed with water and brine solution, dried over anhydrous sodium sulfate, filtered and evaporated the solvent completely under reduced pressure to yield tert-butyl N-[6-[4-(4-anilino-3-carbamoyl-6-quinolyl)-1-piperidyl]-6-oxo-hexyl]carbamate (173, 250 mg, 390.97 μmol, 109.23% yield) as an brown liquid. LCMS (ES+): m/z 560 [M+H]⁺

Step 2: To a stirred solution of tert-butyl (6-(4-(3-carbamoyl-4-(phenylamino)quinolin-6-yl)piperidin-1-yl)-6-oxohexyl)carbamate (173, 250 mg, 0.446 mmol) in dichloromethane (5 mL) was added Trifluoroacetic acid (1 mL) at 25° C. and stirred the mixture for 1 hr at 25° C. The resulting mixture was concentrated completely under reduced pressure to yield 6-(1-(6-aminohexanoyl) piperidin-4-yl)-4-(phenylamino) quinoline-3-carboxamide (250 mg, crude, TFA salt) as a yellow colored solid. To a stirred solution of 2-[[2-(2,6-dioxo-3-piperidyl)-1,3-dioxo-isoindolin-4-yl]-methyl-amino]acetic acid (153, 80 mg, 231.68 μmol) and crude 6-(1-(6-aminohexanoyl)piperidin-4-yl)-4-(phenylamino)quinoline-3-carboxamide (106.48 mg, 231.68 μmol) in DMF (10.0 mL) was added DIPEA (149.71 mg, 1.16 mmol, 201.77 uL) and HATU (132.14 mg, 347.52 μmol). The resulting solution was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase prep HPLC to yield 4-anilino-6-[1-[6-[[2-[[2-(2,6-dioxo-3-piperidyl)-1,3-dioxo-isoindolin-4-yl]-methyl-amino]acetyl]amino]hexanoyl]-4-piperidyl]quinoline-3-carboxamide (Compound 312, 30 mg, 37.63 μmol, 16.24% yield) as an yellow solid. ¹H NMR (400 MHz, DMSO-d₆) δ 12.10 (s, 1H), 11.08 (d, J=3.5 Hz, 1H), 8.95 (d, J=3.6 Hz, 1H), 8.28 (s, 1H), 7.95-7.88 (m, 3H), 7.85 (s, 1H), 7.78 (s, 1H), 7.65-7.59 (m, 1H), 7.45 (t, J=7.7 Hz, 2H), 7.35-7.29 (m, 3H), 7.27-7.21 (m, 2H), 5.08 (dd, J=12.9, 5.3 Hz, 1H), 4.47 (d, J=12.9 Hz, 1H), 4.18 (d, J=17.4 Hz, 1H), 4.12 (d, J=17.0 Hz, 1H), 3.88 (d, J=13.7 Hz, 3H), 3.12-3.03 (m, 3H), 3.00 (s, 2H), 2.94-2.75 (m, 3H), 2.50 (s, 2H), 2.05-1.95 (m, 1H), 1.67 (t, J=15.8 Hz, 2H), 1.54-1.38 (m, 5H), 1.34-1.24 (m, 2H), 1.23-1.11 (m, 2H). LCMS (ES+): m/z 787 [M+H]⁺

### Synthesis of Compound 313

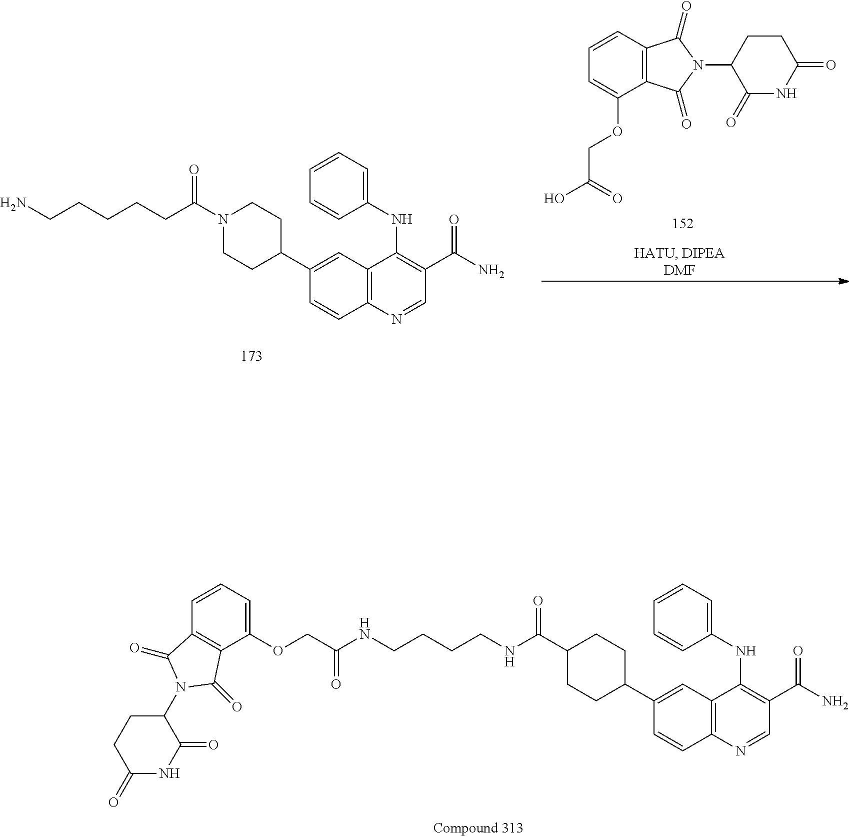 [SMILES: NCCCCCC(=O)N1CCC(c2ccc3ncc(C(N)=O)c(Nc4ccccc4)c3c2)CC1 ; O=C(O)COc1cccc2c1C(=O)N(C1CCC(=O)NC1=O)C2=O ; NC(=O)c1cnc2ccc(C3CCC(C(=O)NCCCCNC(=O)COc4cccc5c4C(=O)N(C4CCC(=O)NC4=O)C5=O)CC3)cc2c1Nc1ccccc1]

To a stirred solution of 2-[2-(2,6-dioxo-3-piperidyl)-1,3-dioxo-isoindolin-4-yl]oxyacetic acid (152, 80 mg, 240.77 μmol) and 6-[1-(6-aminohexanoyl)-4-piperidyl]-4-anilino-quinoline-3-carboxamide (174, 110.65 mg, 240.77 μmol) in DMF (10.0 mL) was added DIPEA (155.59 mg, 1.20 mmol, 209.69 uL) and HATU (137.32 mg, 361.16 μmol). The resulting solution was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[1-[6-[[2-[2-(2,6-dioxo-3-piperidyl)-1,3-dioxo-isoindolin-4-yl]oxyacetyl]amino]hexanoyl]-4-piperidyl]quinoline-3-carboxamide (Compound 313, 25 mg, 31.41 μmol, 13.05% yield) as an yellow solid. LCMS (ES+): m/z 774 [M+H]⁺

### Synthesis of Compound 314

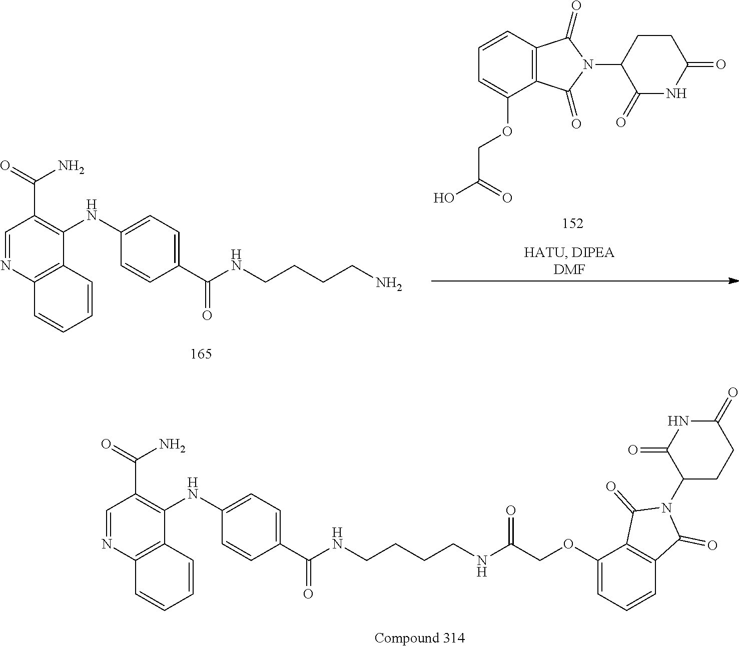 [SMILES: NCCCCNC(=O)c1ccc(Nc2c(C(N)=O)cnc3ccccc23)cc1 ; O=C(O)COc1cccc2c1C(=O)N(C1CCC(=O)NC1=O)C2=O ; NC(=O)c1cnc2ccccc2c1Nc1ccc(C(=O)NCCCCNC(=O)COc2cccc3c2C(=O)N(C2CCC(=O)NC2=O)C3=O)cc1]

To a stirred solution of 4-[4-(4-aminobutylcarbamoyl)anilino]quinoline-3-carboxamide (165, 100 mg, 264.94 μmol) and 2-[2-(2,6-dioxo-3-piperidyl)-1,3-dioxo-isoindolin-4-yl]oxyacetic acid (152, 132.05 mg, 397.42 μmol) in DMF (5 mL) was added DIPEA (171.21 mg, 1.32 mmol, 230.74 uL) and HATU (151.11 mg, 397.42 μmol). The reaction was stirred for 16 hr at 25° C. The reaction mixture was diluted with water (25 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-[4-[4-[[2-[2-(2,6-dioxo-3-piperidyl)-1,3-dioxo-isoindolin-4-yl]oxyacetyl]amino]butylcarbamoyl]anilino]quinoline-3-carboxamide (Compound 314, 8 mg, 11.33 μmol, 4.28% yield) as a yellow solid. ¹H NMR (400 MHz, DMSO-d₆) δ 11.13 (s, 1H), 9.02 (s, 1H), 8.51-8.44 (m, 1H), 8.26 (s, 1H), 8.10 (d, J=8.6 Hz, 1H), 8.04-7.99 (m, 2H), 7.99-7.93 (m, 1H), 7.89-7.83 (m, 2H), 7.83-7.79 (m, 1H), 7.61 (t, J=7.8 Hz, 1H), 7.49 (d, J=7.3 Hz, 1H), 7.40 (d, J=8.5 Hz, 1H), 7.30-7.21 (m, 1H), 5.13 (dd, J=12.9, 5.4 Hz, 1H), 4.78 (s, 2H), 3.23-3.16 (m, 2H), 2.96-2.83 (m, 2H), 2.71-2.61 (m, 3H), 2.38-2.31 (m, 1H), 2.10-1.99 (m, 1H), 1.59-1.42 (m, 4H), 1.24 (s, 1H). LCMS (ES+): m/z 692 [M+H]⁺

### Synthesis of Compound 315

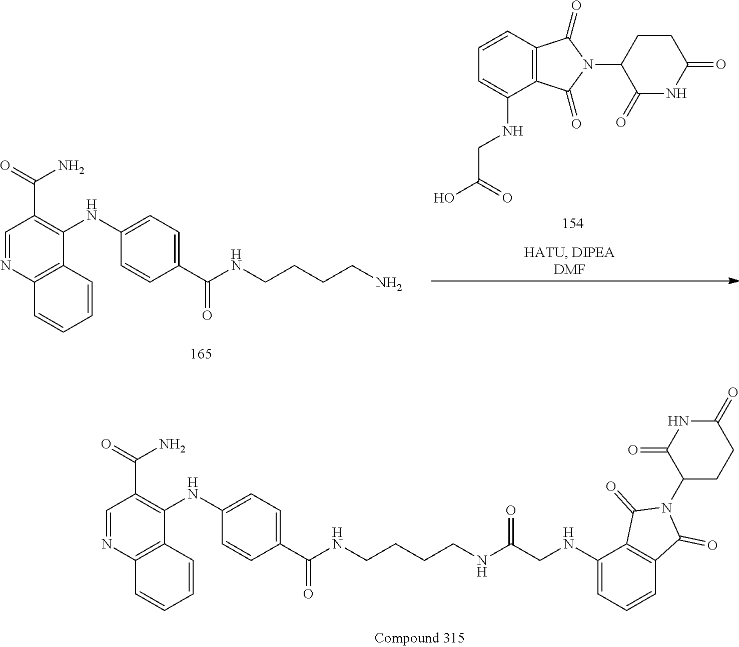 [SMILES: NCCCCNC(=O)c1ccc(Nc2c(C(N)=O)cnc3ccccc23)cc1 ; O=C(O)CNc1cccc2c1C(=O)N(C1CCC(=O)NC1=O)C2=O ; NC(=O)c1cnc2ccccc2c1Nc1ccc(C(=O)NCCCCNC(=O)CNc2cccc3c2C(=O)N(C2CCC(=O)NC2=O)C3=O)cc1]

To a stirred solution of 4-[4-(4-aminobutylcarbamoyl)anilino]quinoline-3-carboxamide (165, 100 mg, 264.94 μmol) and 2-[[2-(2,6-dioxo-3-piperidyl)-1,3-dioxo-isoindolin-4-yl]amino]acetic acid (154, 131.66 mg, 397.42 μmol) in DMF (5 mL) was added DIPEA (171.21 mg, 1.32 mmol, 230.74 uL) and HATU (151.11 mg, 397.42 μmol) Then stirred for 16 hr at 25° C. The reaction mixture was diluted with water (25 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-[4-[4-[[2-[[2-(2,6-dioxo-3-piperidyl)-1,3-dioxo-isoindolin-4-yl]amino]acetyl]amino]butylcarbamoyl]anilino]quinoline-3-carboxamide (Compound 315, 10 mg, 14.19 μmol, 5.36% yield) as a yellow solid. ¹H NMR (400 MHz, DMSO-d₆) δ 11.66 (s, 1H), 11.10 (s, 1H), 9.01 (s, 1H), 8.49 (t, J=4.8 Hz, 1H), 8.26 (s, 1H), 8.16-8.10 (m, 2H), 8.04-7.94 (m, 2H), 7.87 (d, J=8.5 Hz, 2H), 7.82 (s, 1H), 7.66-7.56 (m, 2H), 7.30 (d, J=8.2 Hz, 2H), 7.06 (d, J=7.0 Hz, 1H), 6.99-6.91 (m, 1H), 6.86 (d, J=8.6 Hz, 1H), 5.07 (dd, J=12.9, 5.4 Hz, 1H), 3.92 (d, J=5.1 Hz, 2H), 3.26 (dd, J=12.1, 6.0 Hz, 2H), 3.14 (q, J=6.6, 6.0 Hz, 2H), 2.96-2.82 (m, 2H), 2.71-2.59 (m, 2H), 2.37-2.31 (m, 1H), 2.07-1.95 (m, 2H), 1.56-1.41 (m, 4H). LCMS (ES+): m/z 691 [M+H]⁺

### Synthesis of Compound 316

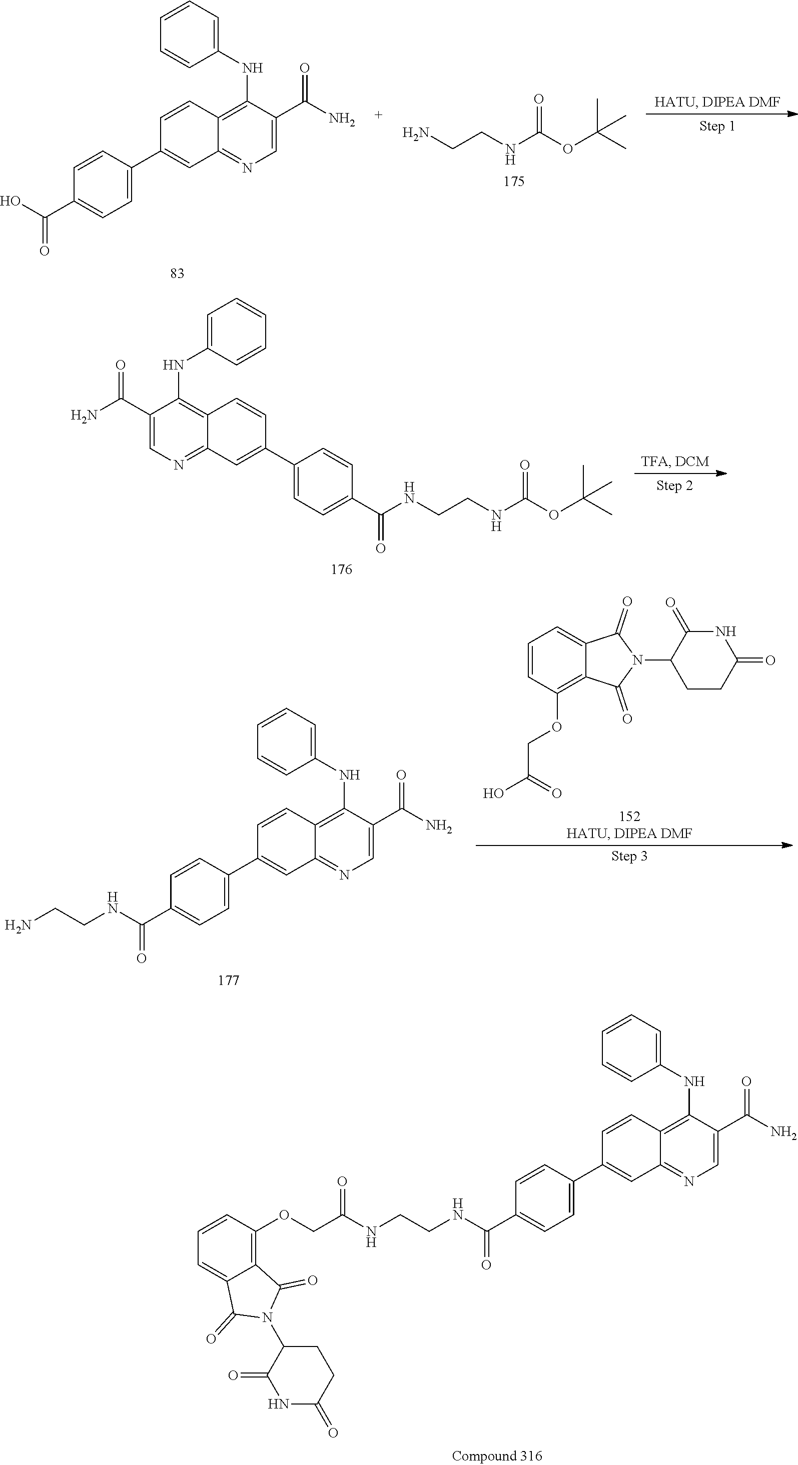 [SMILES: NC(=O)c1cnc2cc(-c3ccc(C(=O)O)cc3)ccc2c1Nc1ccccc1 ; CC(C)(C)OC(=O)NCCN ; CC(C)(C)OC(=O)NCCNC(=O)c1ccc(-c2ccc3c(Nc4ccccc4)c(C(N)=O)cnc3c2)cc1 ; NCCNC(=O)c1ccc(-c2ccc3c(Nc4ccccc4)c(C(N)=O)cnc3c2)cc1 ; O=C(O)COc1cccc2c1C(=O)N(C1CCC(=O)NC1=O)C2=O ; NC(=O)c1cnc2cc(-c3ccc(C(=O)NCCNC(=O)COc4cccc5c4C(=O)N(C4CCC(=O)NC4=O)C5=O)cc3)ccc2c1Nc1ccccc1]

Step 1: To a stirred solution of 4-(3-carbamoyl-4-(phenylamino)quinolin-7-yl)benzoic acid (83, 200 mg, 0.5216 mmol) in DMF (10.0 mL) was added tert-butyl (2-aminoethyl)carbamate (175, 125 mg, 0.7823 mmol), N,N-Diisopropylethylamine (268 mg, 2.08 mmol) and HATU (0.7823 mmol) at room temperature and stirred the resulting solution over night at room temperature. Added water (15 ml) to the reaction mixture and extracted with ethyl acetate (3×15 ml). The combined organic extracts were washed with water and brine solution, dried over anhydrous sodium sulfate, filtered and evaporated the solvent completely under reduced pressure. The resulting solid compound was filtered and washed with water and followed by pet ether to yield tert-butyl (2-(4-(3-carbamoyl-4-(phenylamino)quinolin-7-yl)benzamido)ethyl)carbamate (176, 198 mg, 0.376 mmol, 72.2%) as a off white solid. LCMS (ES+): m/z 526 [M+H]⁺

Step 2: To a stirred solution of tert-butyl (2-(4-(3-carbamoyl-4-(phenylamino)quinolin-7-yl)benzamido)ethyl)carbamate (176, 50 mg, 0.09512 mmol) in Dichloromethane (10.0 mL). Added Trifluoroacetic acid (1.0 mL) and stirred for 30 minutes at room temperature. The resulting reaction mixture was concentrated completely under reduced pressure to yield 7-(4-((2-aminoethyl)carbamoyl)phenyl)-4-(phenylamino)quinoline-3-carboxamide (177, 42.4 mg, 0.09983 mmol, 10%) as a yellow colored solid. The resulting crude product was directly used for next step. LCMS (ES+): m/z 426 [M+H]⁺

Step 3: To a stirred solution of 2-((2-(2,6-dioxopiperidin-3-yl)-1,3-dioxoisoindolin-4-yl)oxy)acetic acid (152, 15 mg, 0.04514 mmol) in DMF (2.0 mL) was added 7-(4-((2-aminoethyl)carbamoyl)phenyl)-4-(phenylamino)quinoline-3-carboxamide (177, 23.0 mg, 0.05416 mmol), N,N-Diisopropylethylamine (29.1 mg, 0.2257 mmol) and HATU (25.7 mg, 0.06770 mmol) at room temperature and stirred over night at room temperature. Added water (15 ml) to the reaction mixture and extracted with ethyl acetate (3×10 mL). The combined organic extracts were washed with water and brine solution, dried over anhydrous sodium sulfate, filtered and evaporated the solvent completely under reduced pressure to yield 7-(4-((2-(2-((2-(2,6-dioxopiperidin-3-yl)-1,3-dioxoisoindolin-4-yl)oxy)acetamido)ethyl)carbamoyl)phenyl)-4-(phenylamino)quinoline-3-carboxamide (Compound 316, 29.8 mg, 0.0349 mmol, 77%) as a pale brown colored oil. LCMS (ES+): m/z 740 [M+H]⁺

### Synthesis of Compound 317

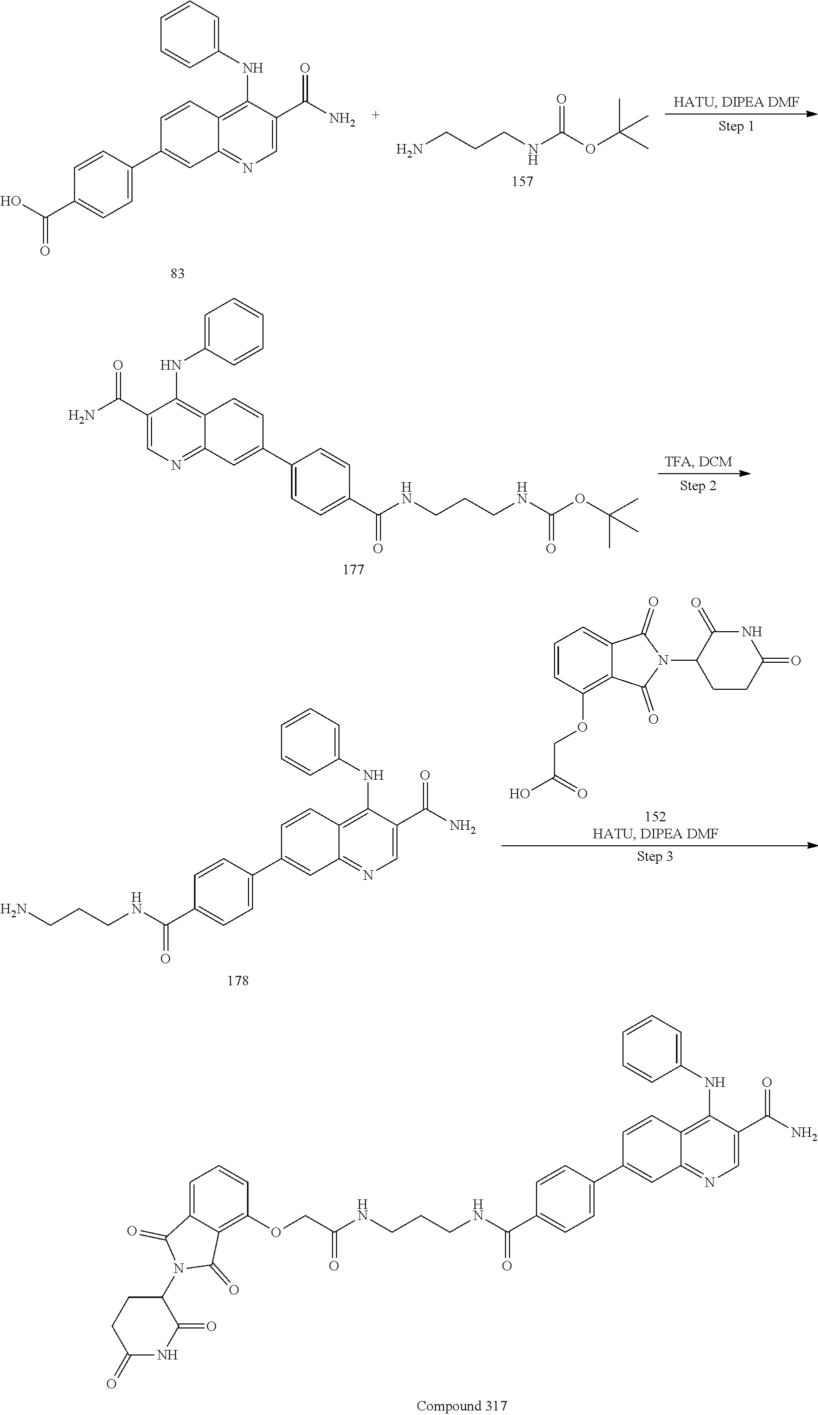 [SMILES: NC(=O)c1cnc2cc(-c3ccc(C(=O)O)cc3)ccc2c1Nc1ccccc1 ; CC(C)(C)OC(=O)NCCCN ; CC(C)(C)OC(=O)NCCCNC(=O)c1ccc(-c2ccc3c(Nc4ccccc4)c(C(N)=O)cnc3c2)cc1 ; NCCCNC(=O)c1ccc(-c2ccc3c(Nc4ccccc4)c(C(N)=O)cnc3c2)cc1 ; O=C(O)COc1cccc2c1C(=O)N(C1CCC(=O)NC1=O)C2=O ; NC(=O)c1cnc2cc(-c3ccc(C(=O)NCCCNC(=O)COc4cccc5c4C(=O)N(C4CCC(=O)NC4=O)C5=O)cc3)ccc2c1Nc1ccccc1]

Step 1: To a stirred solution of 4-(3-carbamoyl-4-(phenylamino)quinolin-7-yl)benzoic acid (83, 200 mg, 0.5216 mmol) in DMF (10.0 mL) was added tert-butyl (3-aminopropyl)carbamate (157, 136 mg, 0.7823 mmol), N,N-Diisopropylethylamine (268 mg, 2.08 mmol) and HATU (297 mg, 0.7823 mmol) at room temperature and stirred the resulting solution over night at room temperature. Added water (15 ml) to the reaction mixture and extracted with ethyl acetate (3×15 ml). The combined organic extracts were washed with water and brine solution, dried over anhydrous sodium sulfate, filtered and evaporated the solvent completely under reduced pressure. The resulting solid compound was filtered and washed with water and followed by pet ether to yield tert-butyl (3-(4-(3-carbamoyl-4-(phenylamino)quinolin-7-yl)benzamido)propyl)carbamate (177, 213 mg, 0.396 mmol, 75.8%) as a off white solid. LCMS (ES+): m/z 540 [M+H]⁺

Step 2: To a stirred solution of tert-butyl (3-(4-(3-carbamoyl-4-(phenylamino)quinolin-7-yl)benzamido)propyl)carbamate (177, 50 mg, 0.09265 mmol) in Dichloromethane (10.0 mL) was added Trifluoroacetic acid (1.0 mL) and stirred for 30 minutes at room temperature. The resulting reaction mixture was concentrated completely under reduced pressure to yield 7-(4-((3-aminopropyl)carbamoyl)phenyl)-4-(phenylamino)quinoline-3-carboxamide (178, 48.8 mg, 0.1110 mmol, 100%) as a yellow colored solid. LCMS (ES+): m/z 440 [M+H]⁺

Step 3: To a stirred solution of 2-((2-(2,6-dioxopiperidin-3-yl)-1,3-dioxoisoindolin-4-yl)oxy)acetic acid (152, 50 mg, 150 μmol) in DMF (5.00 mL) was added 7-(4-((3-aminopropyl)carbamoyl)phenyl)-4-(phenylamino)quinoline-3-carboxamide (178, 65.9 mg, 150 μmol), HATU (85.5 mg, 225 μmol) and N,N-Diisopropylethylamine (96.8 mg, 749 μmol) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 7-(4-((3-(2-((2-(2,6-dioxopiperidin-3-yl)-1,3-dioxoisoindolin-4-yl)oxy)acetamido)propyl)carbamoyl)phenyl)-4-(phenylamino)quinoline-3-carboxamide (Compound 317, 30.8 mg, 40.9 μmol, 27.2%) as a yellow colored solid. LCMS (ES+): m/z 754 [M+H]⁺

### Synthesis of Compound 318

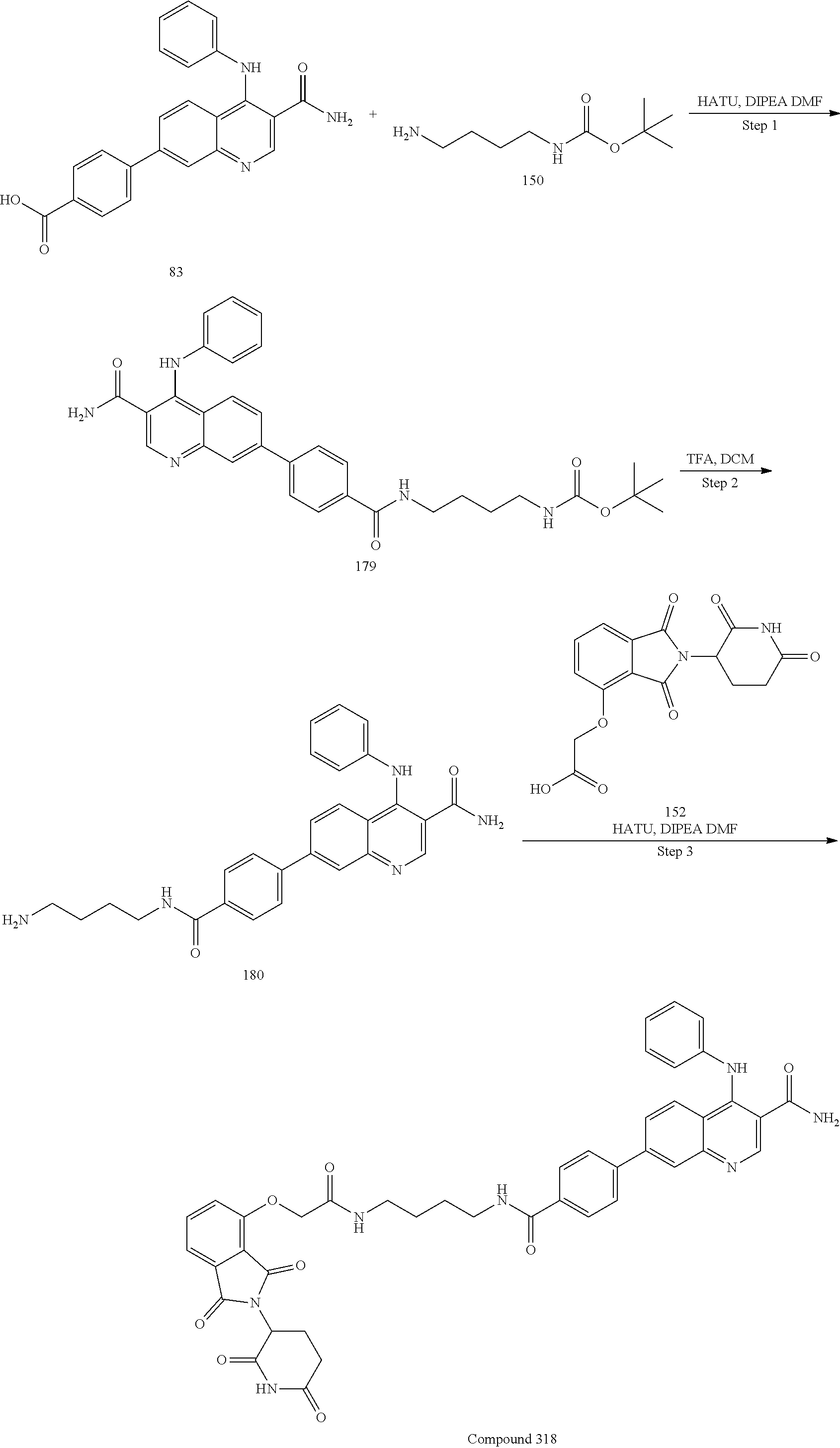 [SMILES: NC(=O)c1cnc2cc(-c3ccc(C(=O)O)cc3)ccc2c1Nc1ccccc1 ; CC(C)(C)OC(=O)NCCCCN ; CC(C)(C)OC(=O)NCCCCNC(=O)c1ccc(-c2ccc3c(Nc4ccccc4)c(C(N)=O)cnc3c2)cc1 ; NCCCCNC(=O)c1ccc(-c2ccc3c(Nc4ccccc4)c(C(N)=O)cnc3c2)cc1 ; O=C(O)COc1cccc2c1C(=O)N(C1CCC(=O)NC1=O)C2=O ; NC(=O)c1cnc2cc(-c3ccc(C(=O)NCCCCNC(=O)COc4cccc5c4C(=O)N(C4CCC(=O)NC4=O)C5=O)cc3)ccc2c1Nc1ccccc1]

Step 1: To a stirred solution of 4-(3-carbamoyl-4-(phenylamino)quinolin-7-yl)benzoic acid (83, 200 mg, 0.5216 mmol) in DMF (10.0 mL) was added tert-butyl (4-aminobutyl)carbamate (150, 147 mg, 0.7823 mmol), N,N-Diisopropylethylamine (268 mg, 2.08 mmol) and HATU (297 mg, 0.7823 mmol) at room temperature and stirred the resulting solution over night at room temperature. Added water (15 ml) to the reaction mixture and extracted with ethyl acetate (3×15 ml). The combined organic extracts were washed with water and brine solution, dried over anhydrous sodium sulfate, filtered and evaporated the solvent completely under reduced pressure. The resulting solid compound was filtered and washed with water and followed by pet ether to yield tert-butyl (4-(4-(3-carbamoyl-4-(phenylamino)quinolin-7-yl)benzamido)butyl)carbamate (179, 196 mg, 0.355 mmol, 68.0%) as a off white solid. LCMS (ES+): m/z 554 [M+H]⁺

Step 2: To a stirred solution of tert-butyl (4-(4-(3-carbamoyl-4-(phenylamino)quinolin-7-yl)benzamido)butyl)carbamate (179, 70 mg, 126 μmol) in Dichloromethane (10.0 mL) was added Trifluoroacetic acid (1.0 mL, 126 μmol) and stirred for 30 minutes at room temperature. The resulting reaction mixture was concentrated completely under reduced pressure to yield 7-(4-((4-aminobutyl)carbamoyl)phenyl)-4-(phenylamino)quinoline-3-carboxamide (180, 51.7 mg, 126 μmol, 100%) as a yellow colored solid. LCMS (ES+): m/z 454 [M+H]⁺

Step 3: To a stirred solution of 2-((2-(2,6-dioxopiperidin-3-yl)-1,3-dioxoisoindolin-4-yl)oxy)acetic acid (152, 50 mg, 150 μmol) in DMF (5.00 mL) was added 7-(4-((4-aminobutyl)carbamoyl)phenyl)-4-(phenylamino)quinoline-3-carboxamide (180, 68.0 mg, 150 μmol), HATU (85.5 mg, 225 μmol) and N,N-Diisopropylethylamine (96.8 mg, 749 μmol) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 7-(4-((4-(2-((2-(2,6-dioxopiperidin-3-yl)-1,3-dioxoisoindolin-4-yl)oxy)acetamido)butyl)carbamoyl)phenyl)-4-(phenylamino)quinoline-3-carboxamide Compound 318, 32.2 mg, 41.9 μmol, 28.0%) as a yellow colored solid. LCMS (ES+): m/z 768

### Synthesis of Compound 319

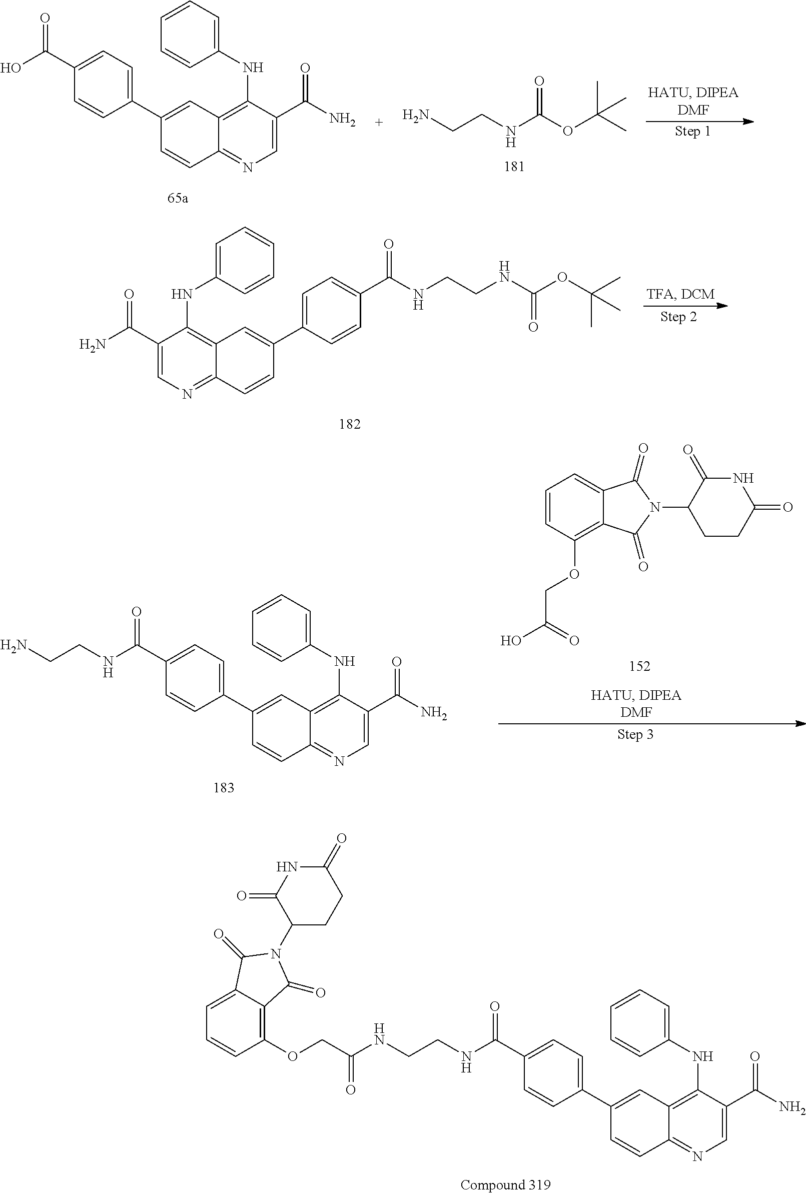 [SMILES: NC(=O)c1cnc2ccc(-c3ccc(C(=O)O)cc3)cc2c1Nc1ccccc1 ; CC(C)(C)OC(=O)NCCN ; CC(C)(C)OC(=O)NCCNC(=O)c1ccc(-c2ccc3ncc(C(N)=O)c(Nc4ccccc4)c3c2)cc1 ; NCCNC(=O)c1ccc(-c2ccc3ncc(C(N)=O)c(Nc4ccccc4)c3c2)cc1 ; O=C(O)COc1cccc2c1C(=O)N(C1CCC(=O)NC1=O)C2=O ; NC(=O)c1cnc2ccc(-c3ccc(C(=O)NCCNC(=O)COc4cccc5c4C(=O)N(C4CCC(=O)NC4=O)C5=O)cc3)cc2c1Nc1ccccc1]

Step 1: To a stirred solution of 4-(3-carbamoyl-4-(phenylamino)quinolin-6-yl)benzoic acid (65a, 200 mg, 0.5216 mmol) in DMF (10.0 mL) was added tert-butyl (2-aminoethyl)carbamate (181, 100 mg, 0.6259 mmol), N,N-Diisopropylethylamine (336 mg, 2.60 mmol) and HATU (297 mg, 0.7823 mmol) at room temperature and stirred overnight at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by column chromatography on silica eluted with 10% methanol in dichloromethane to yield tert-butyl (2-(4-(3-carbamoyl-4-(phenylamino)quinolin-6-yl)benzamido)ethyl)carbamate (182, 98 mg, 0.378 mmol, 72.2%) as a pale yellow colored solid.

Step 2: To a stirred solution of tert-butyl (2-(4-(3-carbamoyl-4-(phenylamino)quinolin-6-yl)benzamido)ethyl)carbamate (182, 50 mg, 0.09512 mmol) in Dichloromethane (10.0 mL) was added Trifluoroacetic acid (1.0 mL, 129 μmol) and stirred for 30 minutes at room temperature. The resulting reaction mixture was concentrated completely under reduced pressure to yield 6-(4-((2-aminoethyl)carbamoyl)phenyl)-4-(phenylamino)quinoline-3-carboxamide (183, 43.8 mg, 0.1029 mmol, 18%) as a yellow colored solid. LCMS (ES+): m/z 426 [M+H]⁺

Step 3: To a stirred solution of 2-((2-(2,6-dioxopiperidin-3-yl)-1,3-dioxoisoindolin-4-yl)oxy)acetic acid (152, 50 mg, 150 μmol) in DMF (5.00 mL) was added 6-(4-((2-aminoethyl)carbamoyl)phenyl)-4-(phenylamino)quinoline-3-carboxamide (183, 63.8 mg, 150 μmol), HATU (85.5 mg, 225 μmol) and N,N-Diisopropylethylamine (96.8 mg, 749 μmol) at room temperature and stirred for over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 6-(4-((2-(2-((2-(2,6-dioxopiperidin-3-yl)-1,3-dioxoisoindolin-4-yl)oxy)acetamido)ethyl)carbamoyl)phenyl)-4-(phenylamino)quinoline-3-carboxamide (Compound 319, 44.3 mg, 59.9 μmol, 40.2%) as a yellow colored solid. LCMS (ES+): m/z 740 [M+H]⁺

### Synthesis of Compound 320

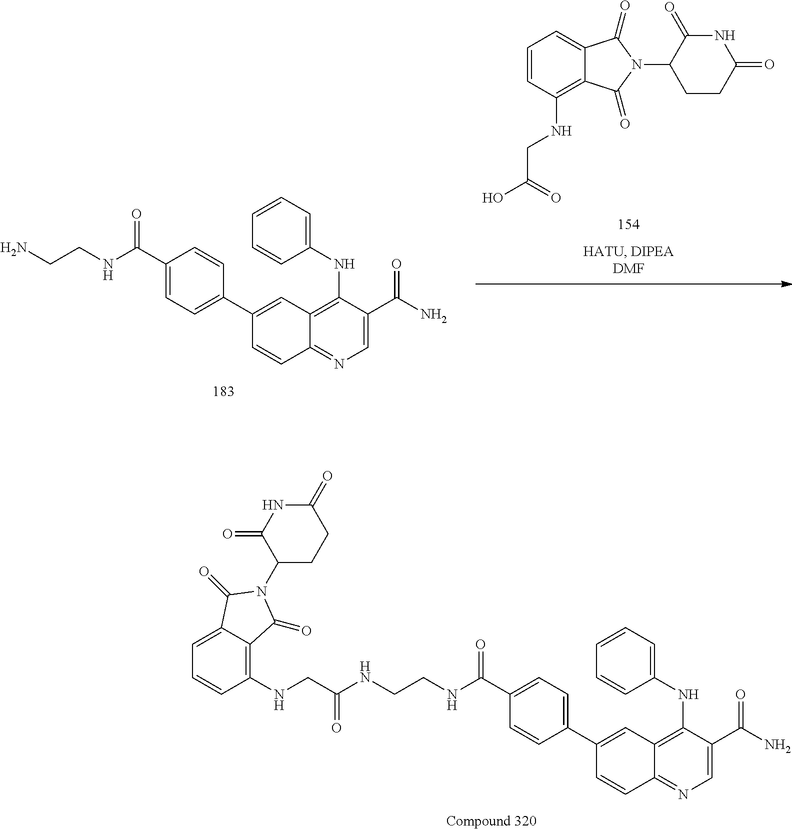 [SMILES: NCCNC(=O)c1ccc(-c2ccc3ncc(C(N)=O)c(Nc4ccccc4)c3c2)cc1 ; O=C(O)CNc1cccc2c1C(=O)N(C1CCC(=O)NC1=O)C2=O ; NC(=O)c1cnc2ccc(-c3ccc(C(=O)NCCNC(=O)CNc4cccc5c4C(=O)N(C4CCC(=O)NC4=O)C5=O)cc3)cc2c1Nc1ccccc1]

To a stirred solution of 2-((2-(2,6-dioxopiperidin-3-yl)-1,3-dioxoisoindolin-4-yl)amino)acetic acid (154, 40 mg, 0.1207 mmol) in DMF (10.0 mL) was added 6-(4-((2-aminoethyl)carbamoyl)phenyl)-4-(phenylamino)quinoline-3-carboxamide (183, 56.4 mg, 0.1327 mmol), HATU (69 mg, 0.181 mmol) and N,N-Diisopropylethylamine (0.5 mL) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by reverse phase preparative HPLC to yield 6-(4-((2-(2-((2-(2,6-dioxopiperidin-3-yl)-1,3-dioxoisoindolin-4-yl)amino)acetamido)ethyl)carbamoyl)phenyl)-4-(phenylamino)quinoline-3-carboxamide (Compound 320, 39.1 mg, 0.05293 mmol, 43.8%) as a yellow colored solid. LCMS (ES+): m/z 739 [M+H]⁺

### Synthesis of Compound 321

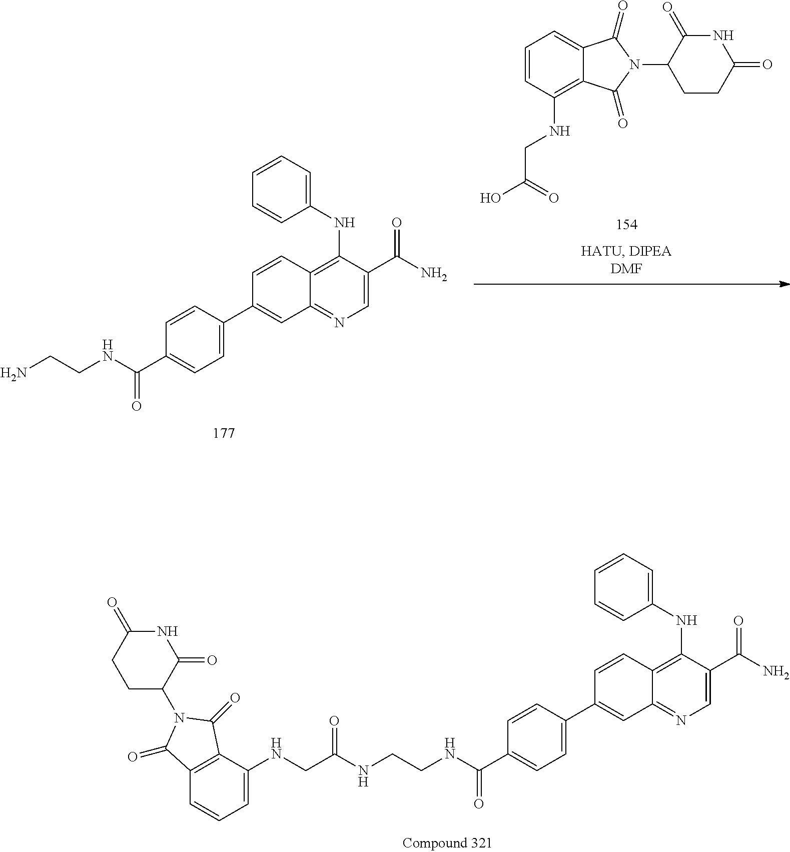 [SMILES: NCCNC(=O)c1ccc(-c2ccc3c(Nc4ccccc4)c(C(N)=O)cnc3c2)cc1 ; O=C(O)CNc1cccc2c1C(=O)N(C1CCC(=O)NC1=O)C2=O ; NC(=O)c1cnc2cc(-c3ccc(C(=O)NCCNC(=O)CNc4cccc5c4C(=O)N(C4CCC(=O)NC4=O)C5=O)cc3)ccc2c1Nc1ccccc1]

To a stirred solution of 2-((2-(2,6-dioxopiperidin-3-yl)-1,3-dioxoisoindolin-4-yl)amino)acetic acid (154, 40 mg, 0.1207 mmol) in DMF (5.00 mL) was added 7-(4-((2-aminoethyl)carbamoyl)phenyl)-4-(phenylamino)quinoline-3-carboxamide (177, 56.4 mg, 0.1327 mmol), HATU (69 mg, 0.181 mmol) and N,N-Diisopropylethylamine (0.5 mL) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 7-(4-((2-(2-((2-(2,6-dioxopiperidin-3-yl)-1,3-dioxoisoindolin-4-yl)amino)acetamido)ethyl)carbamoyl)phenyl)-4-(phenylamino)quinoline-3-carboxamide (Compound 321, 24.2 mg, 0.03284 mmol, 27.1%) as a yellow colored solid. LCMS (ES+): m/z 739 [M+H]⁺

### Synthesis of Compound 322

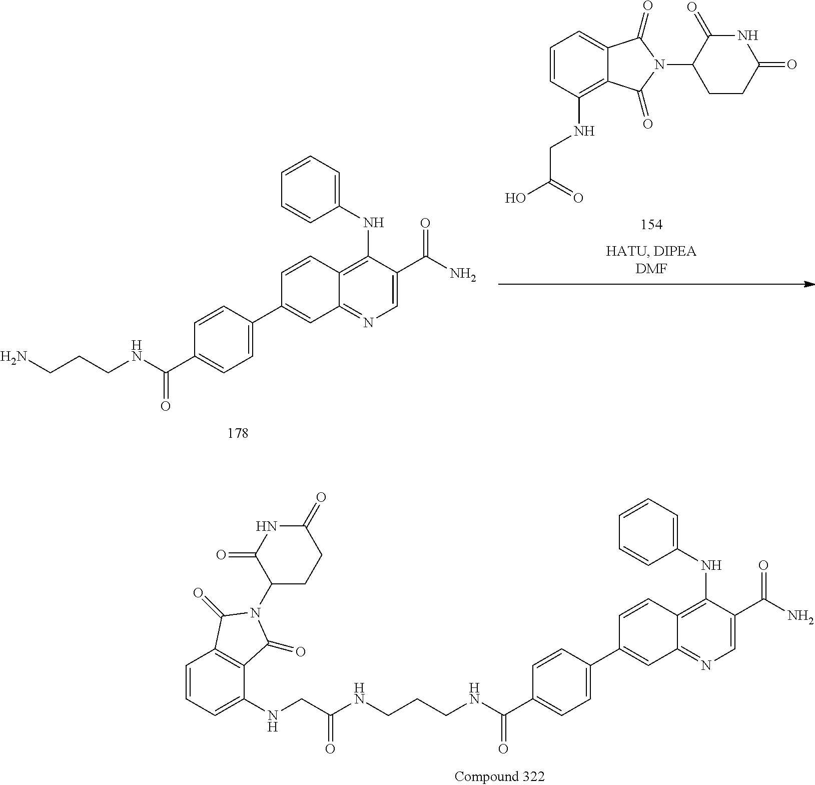 [SMILES: NCCCNC(=O)c1ccc(-c2ccc3c(Nc4ccccc4)c(C(N)=O)cnc3c2)cc1 ; O=C(O)CNc1cccc2c1C(=O)N(C1CCC(=O)NC1=O)C2=O ; NC(=O)c1cnc2cc(-c3ccc(C(=O)NCCCNC(=O)CNc4cccc5c4C(=O)N(C4CCC(=O)NC4=O)C5=O)cc3)ccc2c1Nc1ccccc1]

To a stirred solution of 2-((2-(2,6-dioxopiperidin-3-yl)-1,3-dioxoisoindolin-4-yl)amino)acetic acid (154, 40 mg, 0.1207 mmol) in DMF (5.00 mL) was added 7-(4-((3-aminopropyl)carbamoyl)phenyl)-4-(phenylamino)quinoline-3-carboxamide (178, 53.0 mg, 0.1207 mmol), HATU (69 mg, 0.181 mmol) and N,N-Diisopropylethylamine (0.5 mL) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 7-(4-((3-(2-((2-(2,6-dioxopiperidin-3-yl)-1,3-dioxoisoindolin-4-yl)amino)acetamido)propyl)carbamoyl)phenyl)-4-(phenylamino)quinoline-3-carboxamide (Compound 322, 42.5 mg, 0.05647 mmol, 46.8%) as a yellow colored solid. LCMS (ES+): m/z 753 [M+H]⁺

### Synthesis of Compound 323

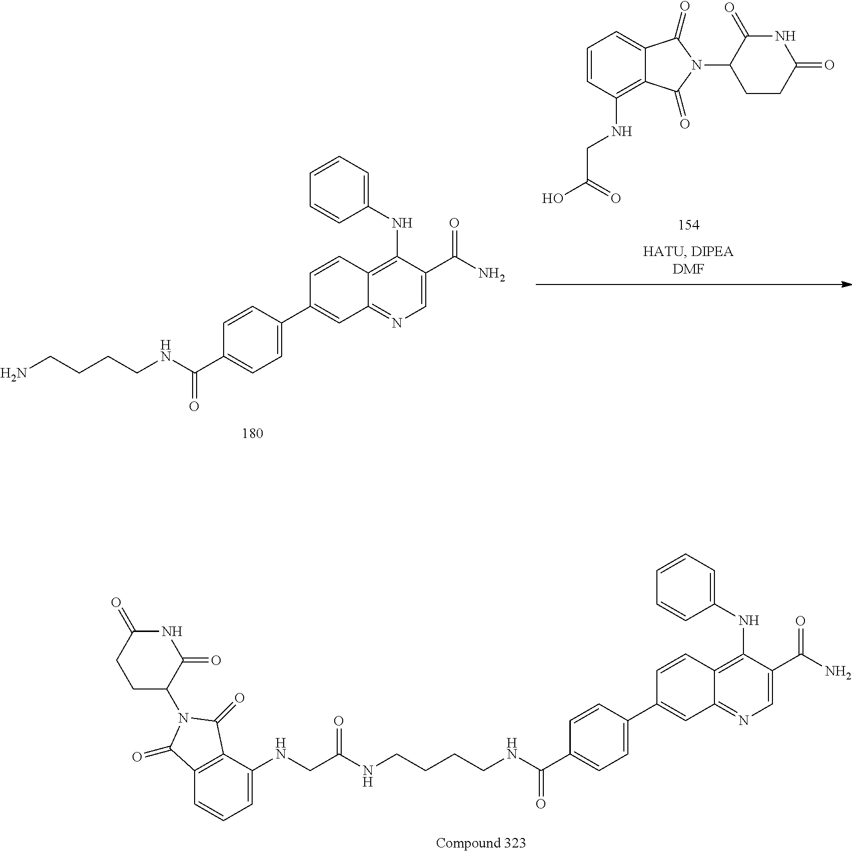 [SMILES: NCCCCNC(=O)c1ccc(-c2ccc3c(Nc4ccccc4)c(C(N)=O)cnc3c2)cc1 ; O=C(O)CNc1cccc2c1C(=O)N(C1CCC(=O)NC1=O)C2=O ; NC(=O)c1cnc2cc(-c3ccc(C(=O)NCCCCNC(=O)CNc4cccc5c4C(=O)N(C4CCC(=O)NC4=O)C5=O)cc3)ccc2c1Nc1ccccc1]

To a stirred solution of 2-((2-(2,6-dioxopiperidin-3-yl)-1,3-dioxoisoindolin-4-yl)amino)acetic acid (154, 40 mg, 0.1207 mmol) in DMF (10.00 mL) was added 7-(4-((4-aminobutyl)carbamoyl)phenyl)-4-(phenylamino)quinoline-3-carboxamide (180, 60.1 mg, 0.1327 mmol), HATU (69 mg, 0.181 mmol) and N,N-Diisopropylethylamine (0.5 mL) at room temperature and stirred for overnight at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 7-(4-((4-(2-((2-(2,6-dioxopiperidin-3-yl)-1,3-dioxoisoindolin-4-yl)amino)acetamido)butyl)carbamoyl)phenyl)-4-(phenylamino)quinoline-3-carboxamide (Compound 323, 28.5 mg, 0.03729 mmol, 30.8%) as a yellow colored solid. LCMS (ES+): m/z 767 [M+H]⁺

### Synthesis of Compound 324

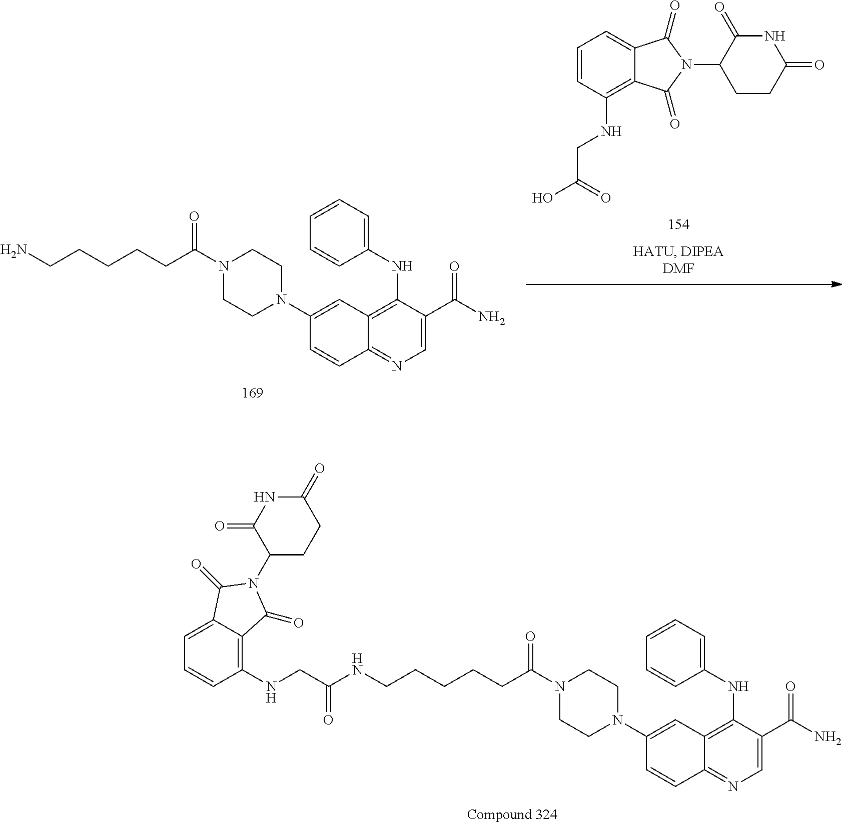 [SMILES: O=C(O)CNc1cccc2c1C(=O)N(C1CCC(=O)NC1=O)C2=O ; NCCCCCC(=O)N1CCN(c2ccc3ncc(C(N)=O)c(Nc4ccccc4)c3c2)CC1 ; NC(=O)c1cnc2ccc(N3CCN(C(=O)CCCCCNC(=O)CNc4cccc5c4C(=O)N(C4CCC(=O)NC4=O)C5=O)CC3)cc2c1Nc1ccccc1]

To a stirred solution of 2-((2-(2,6-dioxopiperidin-3-yl)-1,3-dioxoisoindolin-4-yl)amino)acetic acid (154, 50 mg, 0.1509 mmol) in DMF (10.00 mL) was added 6-(4-(6-aminohexanoyl)piperazin-1-yl)-4-(phenylamino)quinoline-3-carboxamide (169, 76.4 mg, 0.1659 mmol), HATU (86 mg, 0.225 mmol) and N,N-Diisopropylethylamine (0.5 mL) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 6-(4-(6-(2-((2-(2,6-dioxopiperidin-3-yl)-1,3-dioxoisoindolin-4-yl)amino)acetamido)hexanoyl)piperazin-1-yl)-4-(phenylamino)quinoline-3-carboxamide (Compound 324, 19.1 mg, 0.02480 mmol, 16.4%) as a yellow colored solid. LCMS (ES+): m/z 774 [M+H]⁺

### Synthesis of Compound 325

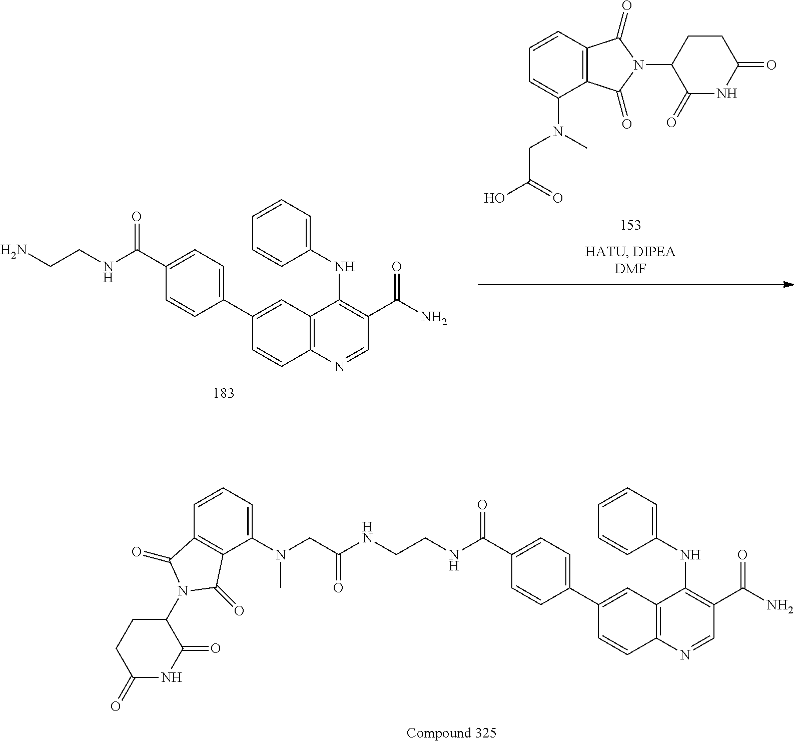 [SMILES: CN(CC(=O)O)c1cccc2c1C(=O)N(C1CCC(=O)NC1=O)C2=O ; NCCNC(=O)c1ccc(-c2ccc3ncc(C(N)=O)c(Nc4ccccc4)c3c2)cc1 ; CN(CC(=O)NCCNC(=O)c1ccc(-c2ccc3ncc(C(N)=O)c(Nc4ccccc4)c3c2)cc1)c1cccc2c1C(=O)N(C1CCC(=O)NC1=O)C2=O]

To a stirred solution of 2-[[2-(2,6-dioxo-3-piperidyl)-1,3-dioxo-isoindolin-4-yl]-methyl-amino]acetic acid (153, 50 mg, 144.80 μmol) in DMF (10.0 mL) was added 6-[4-(2-aminoethylcarbamoyl)phenyl]-4-anilino-quinoline-3-carboxamide (183, 67.77 mg, 159.28 μmol), N,N-Diisopropylethyl amine (93.57 mg, 724.00 μmol, 126.11 uL) and HATU (82.59 mg, 217.20 μmol) at rt. The resulting solution was stirred for 16 hr at rt. Added water (20 mL) and extracted with ethyl acetate (3×20 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[4-[2-[[2-[[2-(2,6-dioxo-3-piperidyl)-1,3-dioxo-isoindolin-4-yl]-methyl-amino]acetyl]amino]ethylcarbamoyl]phenyl]quinoline-3-carboxamide (Compound 325, 40 mg, 51.10 μmol, 35.29% yield) as an yellow solid. LCMS (ES+): m/z 753 [M+H]⁺

### Synthesis of Compound 326

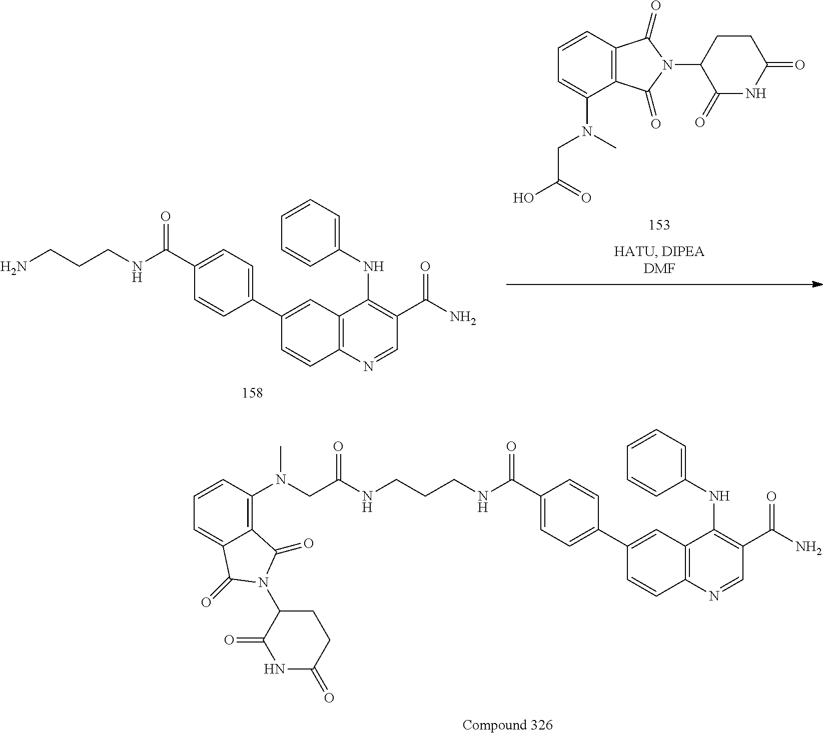 [SMILES: CN(CC(=O)O)c1cccc2c1C(=O)N(C1CCC(=O)NC1=O)C2=O ; NCCCNC(=O)c1ccc(-c2ccc3ncc(C(N)=O)c(Nc4ccccc4)c3c2)cc1 ; CN(CC(=O)NCCCNC(=O)c1ccc(-c2ccc3ncc(C(N)=O)c(Nc4ccccc4)c3c2)cc1)c1cccc2c1C(=O)N(C1CCC(=O)NC1=O)C2=O]

To a stirred solution of 2-[[2-(2,6-dioxo-3-piperidyl)-1,3-dioxo-isoindolin-4-yl]-methyl-amino]acetic acid (153, 50 mg, 144.80 μmol) and 6-[4-(3-aminopropylcarbamoyl)phenyl]-4-anilino-quinoline-3-carboxamide (158, 63.64 mg, 144.80 μmol) in DMF (10 mL) was added N,N-Diisopropylethyl amine (93.57 mg, 724.00 μmol, 126.10 uL) and HATU (82.59 mg, 217.20 μmol) at rt. The resulting solution was stirred for 16 hr at rt. Added water (20 mL) and extracted with ethyl acetate (3×20 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[4-[3-[[2-[[2-(2,6-dioxo-3-piperidyl)-1,3-dioxo-isoindolin-4-yl]-methyl-amino]acetyl]amino]propylcarbamoyl]phenyl]quinoline-3-carboxamide (Compound 326, 45 mg, 56.61 μmol, 39.09% yield, 96.46% purity) as a yellow solid. LCMS (ES+): m/z 767 [M+H]⁺

### Synthesis of Compound 327

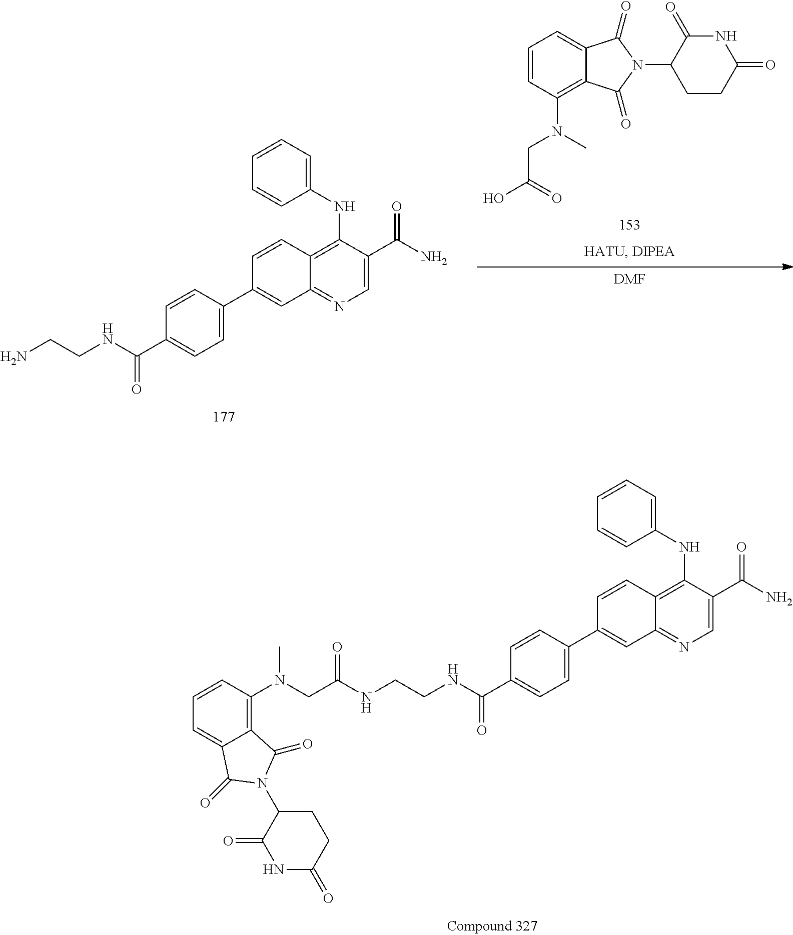

To a stirred solution of 2-((2-(2,6-dioxopiperidin-3-yl)-1,3-dioxoisoindolin-4-yl)(methyl)amino)acetic acid (153, 50 mg, 0.1447 mmol) in DMF (5.00 mL) was added 7-(4-((2-aminoethyl)carbamoyl)phenyl)-4-(phenylamino)quinoline-3-carboxamide (177, 67.6 mg, 0.1591 mmol), HATU (69 mg, 0.181 mmol) and N,N-Diisopropylethylamine (0.5 mL) at room temperature and stirred over night at room temperature. Added water (15 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 7-(4-((2-(2-((2-(2,6-dioxopiperidin-3-yl)-1,3-dioxoisoindolin-4-yl)(methyl)amino)acetamido)ethyl)carbamoyl)phenyl)-4-(phenylamino)quinoline-3-carboxamide (Compound 327, 29.8 mg, 0.039 mmol, 27.5%) as a yellow colored solid. LCMS (ES+): m/z 753 [M+H]⁺

### Synthesis of Compound 328

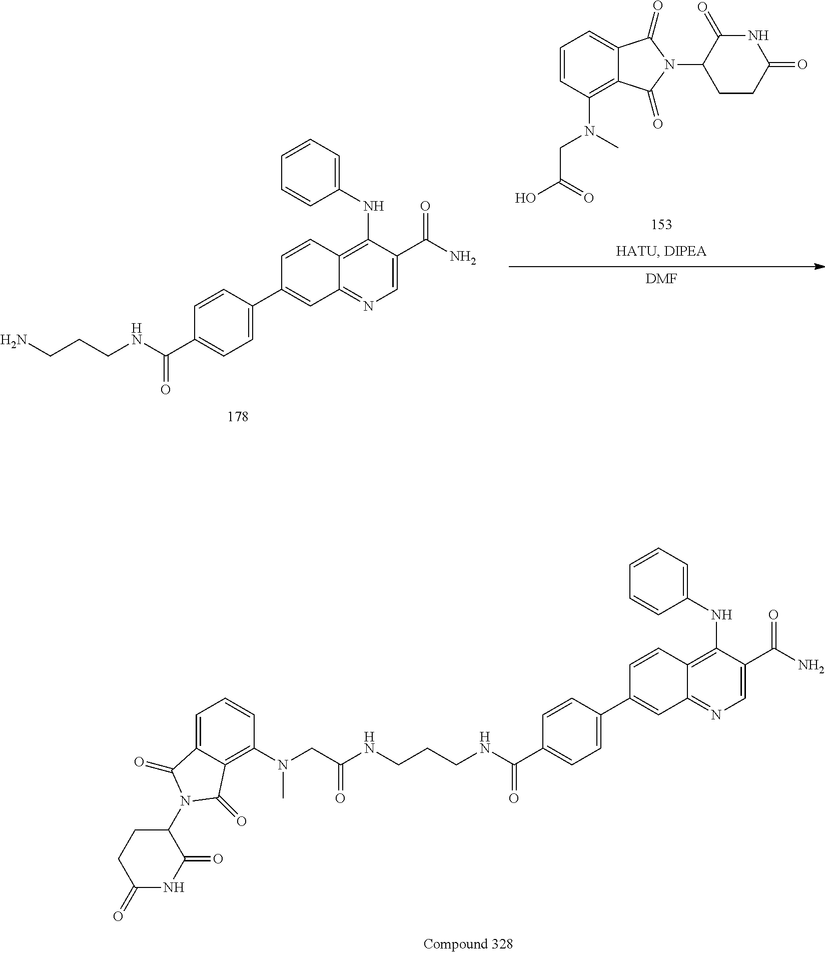

To a stirred solution of 2-((2-(2,6-dioxopiperidin-3-yl)-1,3-dioxoisoindolin-4-yl)(methyl)amino)acetic acid (153, 90 mg, 0.2606 mmol) in DMF (10.0 mL) was added 7-(4-((3-aminopropyl)carbamoyl)phenyl)-4-(phenylamino)quinoline-3-carboxamide (178, 125 mg, 0.2866 mmol), HATU (145 mg, 0.391 mmol) and N,N-Diisopropylethylamine (1.0 mL) at room temperature and stirred over night at room temperature. Added water (15 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 7-(4-((3-(2-((2-(2,6-dioxopiperidin-3-yl)-1,3-dioxoisoindolin-4-yl)(methyl)amino)acetamido)propyl)carbamoyl)phenyl)-4-(phenylamino)quinoline-3-carboxamide (Compound 328, 49.2 mg, 0.064 mmol, 24.7% yield) as a yellow colored solid. LCMS (ES+): m/z 767 [M+H]⁺

### Synthesis of Compound 329

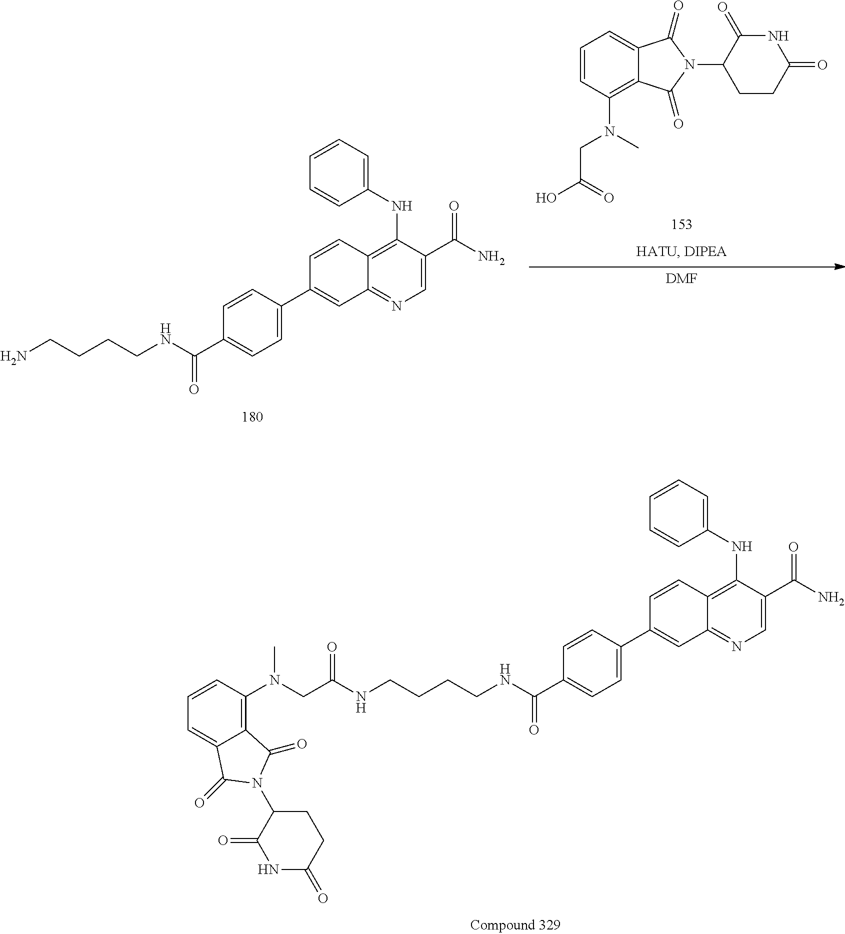

To a stirred solution of 2-((2-(2,6-dioxopiperidin-3-yl)-1,3-dioxoisoindolin-4-yl)(methyl)amino)acetic acid (153, 75 mg, 0.2171 mmol) in DMF (10.0 mL) was added 7-(4-((4-aminobutyl)carbamoyl)phenyl)-4-(phenylamino)quinoline-3-carboxamide (180, 108 mg, 0.2388 mmol), HATU (124 mg, 0.326 mmol) and N,N-Diisopropylethylamine (1.0 mL) at room temperature and stirred over night at room temperature. Added water (15 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield 7-(4-((4-(2-((2-(2,6-dioxopiperidin-3-yl)-1,3-dioxoisoindolin-4-yl)(methyl)amino)acetamido)butyl)carbamoyl)phenyl)-4-(phenylamino)quinoline-3-carboxamide (Compound 329, 47.7 mg, 0.061 mmol, 28.2% yield) as a yellow colored solid. LCMS (ES+): m/z 781 [M+H]⁺

### Synthesis of Compound 330

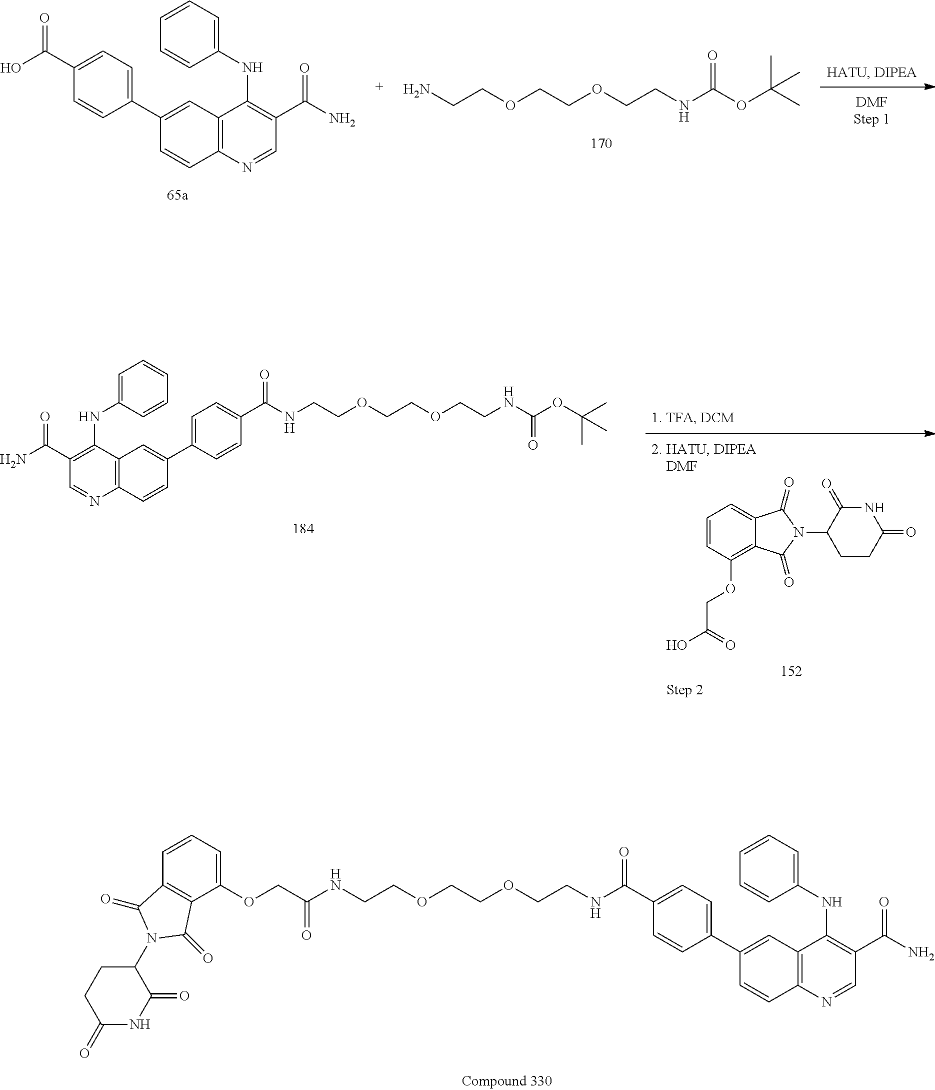 [SMILES: NC(=O)c1cnc2ccc(-c3ccc(C(=O)O)cc3)cc2c1Nc1ccccc1 ; CC(C)(C)OC(=O)NCCOCCOCCN ; "HATU, DIPEA" ; CC(C)(C)OC(=O)NCCOCCOCCNC(=O)c1ccc(-c2ccc3ncc(C(N)=O)c(Nc4ccccc4)c3c2)cc1 ; O=C(O)COc1cccc2c1C(=O)N(C1CCC(=O)NC1=O)C2=O ; NC(=O)c1cnc2ccc(-c3ccc(C(=O)NCCOCCOCCNC(=O)COc4cccc5c4C(=O)N(C4CCC(=O)NC4=O)C5=O)cc3)cc2c1Nc1ccccc1]

Step 1: To a solution of 4-(4-anilino-3-carbamoyl-6-quinolyl)benzoic acid (65a, 300 mg, 782.48 μmol) and tert-butyl N-[2-[2-(2-aminoethoxy)ethoxy]ethyl]carbamate (170, 291.45 mg, 1.17 mmol) in N,N-dimethyl formamide (10 mL) were added N,N-Diisopropylethylamine (505.64 mg, 3.91 mmol, 681.45 uL) followed by HATU (595.04 mg, 1.56 mmol) under nitrogen atmosphere at room temperature. The resulting mixture was stirred at room temperature for 16 h. Ice cold water (15 mL) was added and stirred for 10 minutes, and then extracted with ethyl acetate. Then combined organics were dried over anhydrous sodium sulfate and then concentrated to yield the crude product tert-butyl N-[2-[2-[2-[[4-(4-anilino-3-carbamoyl-6-quinolyl)benzoyl]amino]ethoxy]ethoxy]ethyl]carbamate (184, 320 mg, 521.43 mmol), used without further purification. LCMS (ES+): m/z 614 [M+H]⁺

Step 2: To a stirred solution of crude tert-butyl N-[2-[2-[2-[[4-(4-anilino-3-carbamoyl-6-quinolyl)benzoyl]amino]ethoxy]ethoxy]ethyl]carbamate (184, 150 mg, 244.42 μmol) in anhydrous Dichloromethane (5 mL) was added Trifluoroacetic acid (2.96 g, 25.96 mmol, 2.0 mL) at 0° C. The reaction mixture was stirred at room temperature for 3 hours. Then reaction mixture was concentrated completely and this crude was taken as such for the next step. To the stirred solution of above crude in N,N-Dimethylformamide (6 mL) was added 2-[2-(2,6-dioxo-3-piperidyl)-1,3-dioxo-isoindolin-4-yl]oxyacetic acid (152, 121.82 mg, 366.63 μmol) followed by HATU (139.40 mg, 366.63 μmol) and N,N-Diisopropylethylamine (157.94 mg, 1.22 mmol, 212.86 uL). The reaction mixture was stirred at room temperature for 16 hours. Ice cold water (15 mL) was added and stirred for 10 minutes, and then extracted with ethyl acetate. Then combined organics were dried over anhydrous sodium sulfate and then concentrated to yield the crude product, which was purified by prep-HPLC(SUNFIRE OBD C18(100×30)MM 5μ) Mobile phase: A: 0.1% TFA in water B: ACN) to yield the product 4-anilino-6-[4-[2-[2-[2-[[2-[2-(2,6-dioxo-3-piperidyl)-1,3-dioxo-isoindolin-4-yl]oxyacetyl]amino]ethoxy]ethoxy]ethylcarbamoyl]phenyl]quinoline-3-carboxamide (Compound 330, 38 mg, 44.07 μmol, 18.03% yield) as a pale yellow solid. LCMS (ES+): m/z 828 [M+H]⁺

### Synthesis of Compound 331

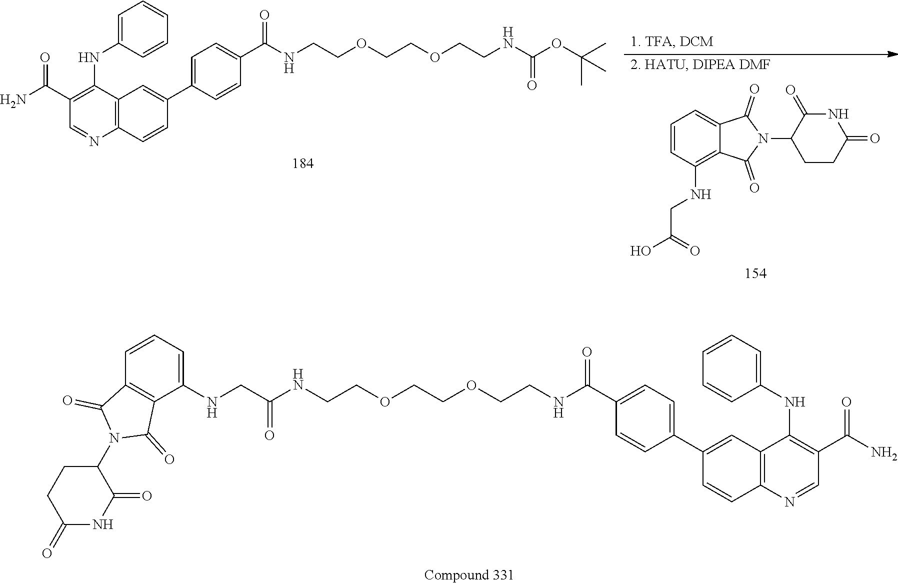 [SMILES: CC(C)(C)OC(=O)NCCOCCOCCNC(=O)c1ccc(-c2ccc3ncc(C(N)=O)c(Nc4ccccc4)c3c2)cc1 ; O=C(O)CNc1cccc2c1C(=O)N(C1CCC(=O)NC1=O)C2=O ; NC(=O)c1cnc2ccc(-c3ccc(C(=O)NCCOCCOCCNC(=O)CNc4cccc5c4C(=O)N(C4CCC(=O)NC4=O)C5=O)cc3)cc2c1Nc1ccccc1]

To a stirred solution of crude tert-butyl N-[2-[2-[2-[[4-(4-anilino-3-carbamoyl-6-quinolyl)benzoyl]amino]ethoxy]ethoxy]ethyl]carbamate (184, 150 mg, 244.42 μmol) in anhydrous Dichloromethane (5 mL) was added Trifluoroacetic acid (2.96 g, 25.96 mmol, 2.0 mL) at 0° C. Then reaction mixture was stirred at room temperature for 3 hours. The reaction mixture was concentrated completely and this crude was taken as such for the next step. To the stirred solution of above crude in N,N-Dimethylformamide (8 mL) was added 2-[[2-(2,6-dioxo-3-piperidyl)-1,3-dioxo-isoindolin-4-yl]amino]acetic acid (154, 121.46 mg, 366.63 μmol) followed by HATU (139.40 mg, 366.63 μmol) and N,N-Diisopropylethylamine (157.94 mg, 1.22 mmol, 212.86 uL). The reaction mixture stirred at room temperature for 16 hours. Ice cold water (15 mL) was added and stirred for 10 minutes, and then extracted with ethyl acetate. The combined organics were dried over anhydrous sodium sulfate and then concentrated to yield the crude product, which was purified by prep-HPLC(SUNFIRE OBD C18(100×30)MM 5μ) Mobile phase: A: 0.1% TFA in water B: ACN) to yield the product 4-anilino-6-[4-[2-[2-[2-[[2-[[2-(2,6-dioxo-3-piperidyl)-1,3-dioxo-isoindolin-4-yl]amino]acetyl]amino]ethoxy]ethoxy]ethylcarbamoyl]phenyl]quinoline-3-carboxamide (Compound 331, 28 mg, 29.80 μmol, 12.19% yield) as a pale yellow solid. LCMS (ES+): m/z 827 [M+H]⁺

### Synthesis of Compound 332

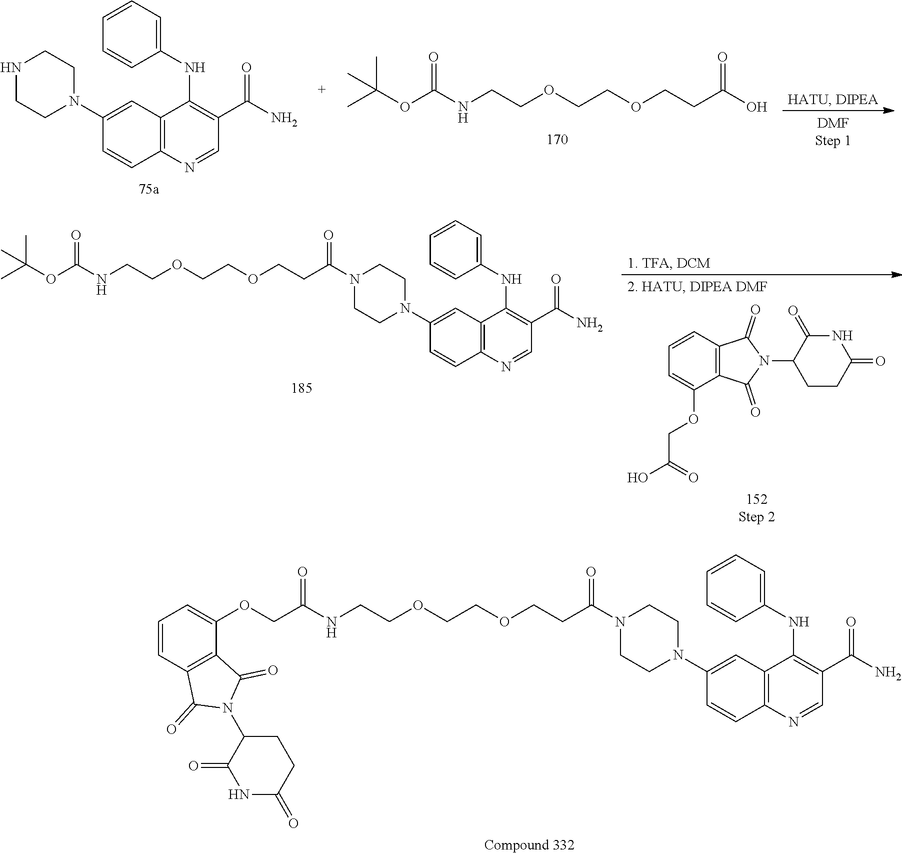 [SMILES: NC(=O)c1cnc2ccc(N3CCNCC3)cc2c1Nc1ccccc1 ; CC(C)(C)OC(=O)NCCOCCOCCC(=O)O ; CC(C)(C)OC(=O)NCCOCCOCCC(=O)N1CCN(c2ccc3ncc(C(N)=O)c(Nc4ccccc4)c3c2)CC1 ; O=C(O)COc1cccc2c1C(=O)N(C1CCC(=O)NC1=O)C2=O ; NC(=O)c1cnc2ccc(N3CCN(C(=O)CCOCCOCCNC(=O)COc4cccc5c4C(=O)N(C4CCC(=O)NC4=O)C5=O)CC3)cc2c1Nc1ccccc1]

Step 1: To a stirred solution of 4-anilino-6-piperazin-1-yl-quinoline-3-carboxamide (75a, 100 mg, 287.84 μmol) and 3-[2-[2-(tert-butoxycarbonylamino)ethoxy]ethoxy]propanoic acid (170, 87.80 mg, 316.63 μmol) in DMF (10.0 mL) was added DIPEA (186.01 mg, 1.44 mmol, 250.68 uL) and HATU (164.17 mg, 431.76 μmol). The resulting solution was stirred for 16 h at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure to yield tert-butyl N-[2-[2-[3-[4-[4-anilino-3-carbamoyl-6-quinolyl)piperazin-1-yl]-3-oxo-propoxy]ethoxy]ethyl]carbamate (185, 130 mg, 139.28 μmol, 48.39% yield, 65% purity) as a brown solid. LCMS (ES+): m/z 607 [M+H]⁺

Step 2: To a stirred solution of tert-butyl N-[2-[2-[3-[4-(4-anilino-3-carbamoyl-6-quinolyl)piperazin-1-yl]-3-oxo-propoxy]ethoxy]ethyl]carbamate (185, 130 mg, 214.27 μmol) in Dichloromethane (5.00 mL) was added Trifluoroacetic acid (1.48 g, 12.98 mmol, 1.0 mL) and stirred for 1 hr at 25° C. The resulting mixture was concentrated completely under reduced pressure to yield crude 6-[4-[3-[2-(2-aminoethoxy)ethoxy]propanoyl]piperazin-1-yl]-4-anilino-quinoline-3-carboxamide (105 mg, 207.27 μmol, 96.73% yield, 061) as a yellow solid.

To a stirred solution of 2-[2-(2,6-dioxo-3-piperidyl)-1,3-dioxo-isoindolin-4-yl]oxyacetic acid (152, 50 mg, 150.48 μmol) and crude 6-[4-[3-[2-(2-aminoethoxy)ethoxy]propanoyl]piperazin-1-yl]-4-anilino-quinoline-3-carboxamide (91.30 mg, 150.48 μmol) in DMF (5.00 mL) was added DIPEA (97.24 mg, 752.41 μmol, 131.06 uL) and HATU (85.83 mg, 225.72 μmol). The resulting solution was stirred for 16 hr at 25° C. The resulting mixture was diluted with water (20 mL) and extracted with ethyl acetate (3×15 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[4-[3-[2-[2-[[2-[2-(2,6-dioxo-3-piperidyl)-1,3-dioxo-isoindolin-4-yl]oxyacetyl]amino]ethoxy]ethoxy]propanoyl]piperazin-1-yl]quinoline-3-carboxamide (Compound 332, 10.0 mg, 11.02 μmol, 7.32% yield) as a yellow solid. LCMS (ES+): m/z 821 [M+H]⁺

### Synthesis of Compound 333

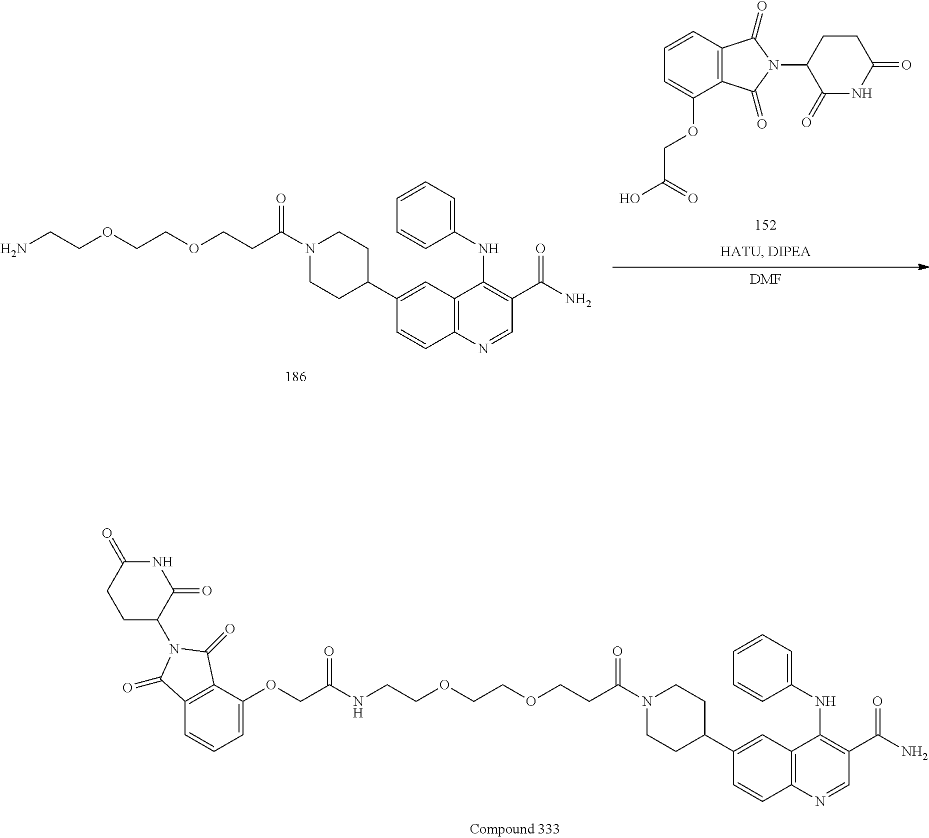

To a stirred solution of 2-[2-(2,6-dioxo-3-piperidyl)-1,3-dioxo-isoindolin-4-yl]oxyacetic acid (152, 70 mg, 210.68 μmol) and 6-[1-[3-[2-(2-aminoethoxy)ethoxy]propanoyl]-4-piperidyl]-4-anilino-quinoline-3-carboxamide (186, 106.52 mg, 210.68 μmol) in DMF (10.0 mL) was added DIPEA (136.14 mg, 1.05 mmol, 183.48 uL) and HATU (120.16 mg, 316.01 μmol). The resulting solution was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase prep HPLC to yield 4-anilino-6-[1-[3-[2-[2-[[2-[2-(2,6-dioxo-3-piperidyl)-1,3-dioxo-isoindolin-4-yl]oxyacetyl]amino]ethoxy]ethoxy]propanoyl]-4-piperidyl]quinoline-3-carboxamide (Compound 333, 10 mg, 11.84 μmol, 5.62% yield) as an yellow solid. LCMS (ES+): m/z 820 [M+H]⁺

### Synthesis of Compound 334

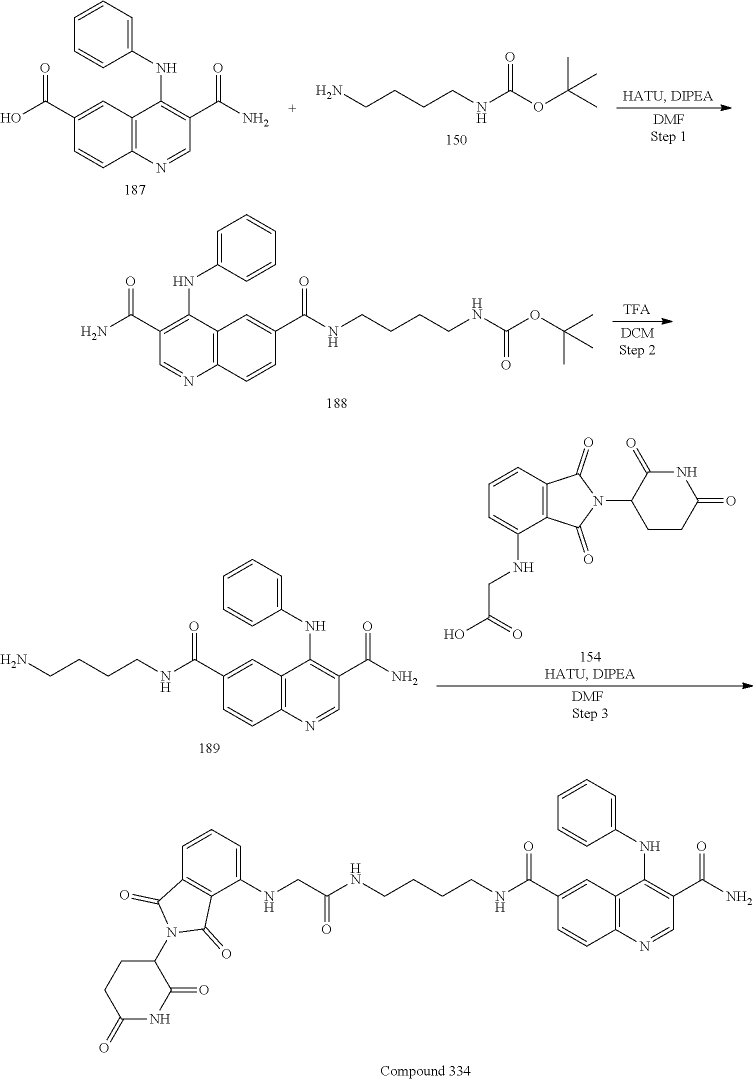

Step 1: An oven dried round bottom flask was charged with a solution of 4-anilino-3-carbamoyl-quinoline-6-carboxylic acid (187, 45 mg, 146.44 μmol) in DMF (2 mL), tert-butyl N-(4-aminobutyl)carbamate (150, 33.08 mg, 175.73 μmol), DIPEA (94.63 mg, 732.20 μmol, 127.53 uL) and HATU (66.81 mg, 175.73 μmol) were added. The reaction mixture was stirred for 16 h at room temperature. The reaction mixture was concentrated under reduced pressure. The crude mixture was purified by reverse phase prep column chromatography to yield tert-butyl N-[4-[(4-anilino-3-carbamoyl-quinoline-6-carbonyl)amino]butyl]carbamate (188, 40 mg, 80.08 μmol, 54.69% yield) as white solid. LCMS (ES+): m/z 478 [M+H]⁺

Step 2: An oven dried round bottom flask was charged with tert-butyl N-[4-[(4-anilino-3-carbamoyl-quinoline-6-carbonyl)amino]butyl]carbamate (188, 40 mg, 83.76 μmol) in Dichloromethane (3 mL), Trifluoroacetic acid (1.48 g, 12.98 mmol, 1 mL) was added and the reaction mixture was stirred for an hour at room temperature. The reaction mixture was concentrated under reduced pressure to yield N6-(4-aminobutyl)-4-anilino-quinoline-3,6-dicarboxamide (189, 30 mg) as brown gummy oil, used without further purification. LCMS (ES+): m/z 378 [M+H]⁺

Step 3: An oven dried round bottom flask was charged with a solution of 2-[[2-(2,6-dioxo-3-piperidyl)-1,3-dioxo-isoindolin-4-yl]amino]acetic acid (154, 30.72 mg, 92.73 μmol) in DMF (5 mL), N6-(4-aminobutyl)-4-anilino-quinoline-3,6-dicarboxamide (189, 35.00 mg, 92.73 μmol), DIPEA (59.92 mg, 463.65 μmol, 80.76 uL) and HATU (42.31 mg, 111.28 μmol) were added. The reaction mixture was stirred for 16 h at room temperature. The reaction mixture was concentrated under reduced pressure. The crude mixture was purified by reverse phase prep column chromatography (Column: HPLC(SUNFIRE OBD C18(100×30)MM 5μ) Mobile phase: A: 0.1% TFA in water B: ACN) to yield 4-anilino-N6-[4-[[2-[[2-(2,6-dioxo-3-piperidyl)-1,3-dioxo-isoindolin-4-yl]amino]acetyl]amino]butyl]quinoline-3,6-dicarboxamide (Compound 334, 22.28 mg, 30.81 μmol, 33.22% yield) as yellow solid. LCMS (ES+): m/z 691 [M+H]⁺

### Synthesis of Compound 335

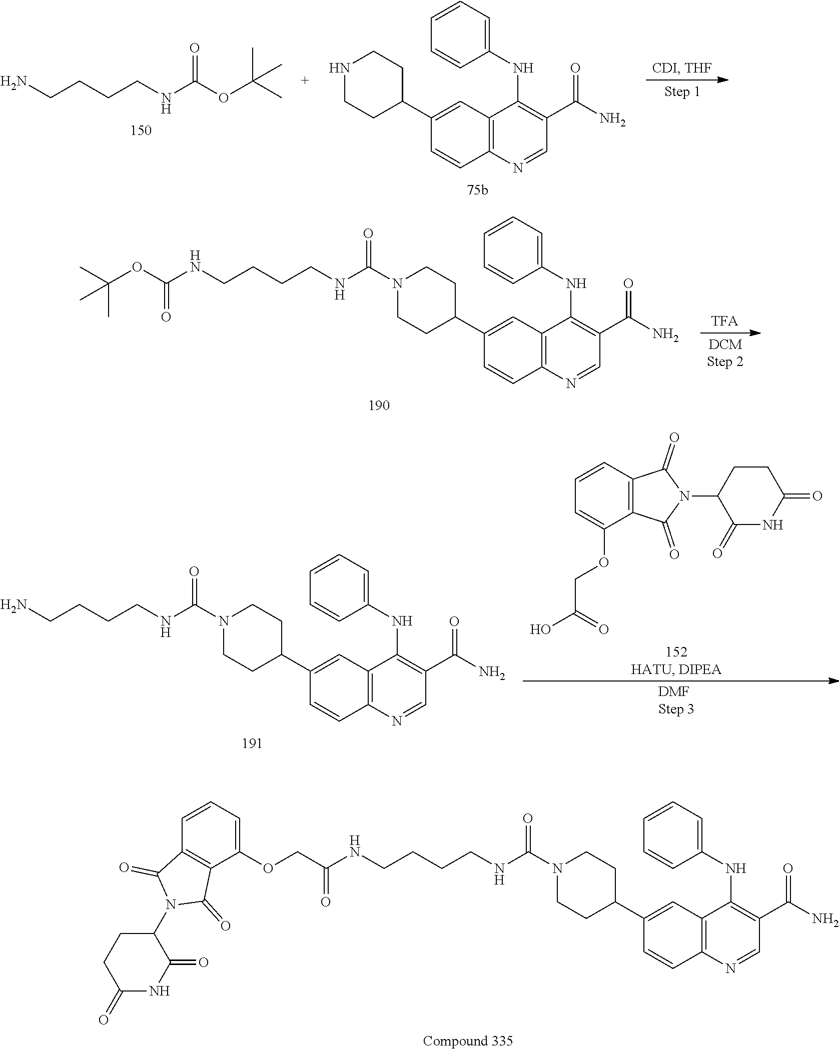

Step 1: To a stirred solution of tert-butyl N-(4-aminobutyl)carbamate (150, 80 mg, 424.93 μmol) in THF (7.0 mL) was added 1,1′-Carbonyldiimidazole (75.79 mg, 467.42 μmol) and stirred for 2 hr at 80° C. The resulting mixture was cooled to ambient temperature and added a solution of 4-anilino-6-(4-piperidyl)quinoline-3-carboxamide (75b, 176.65 mg, 509.92 μmol) in THF (7.0 mL) and stirred for 16 hr at 25° C. Added water to the resulting mixture and extracted with ethyl acetate (2×25 mL). The combined organic extracts were washed with water and brine solution, dried over anhydrous sodium sulfate, filtered and evaporated the solvent completely under reduced pressure. The resulting crude was purified by column chromatography on silica eluted with 5% methanol in dichloromethane to yield tert-butyl N-[4-[[4-(4-anilino-3-carbamoyl-6-quinolyl)piperidine-1-carbonyl]amino]butyl]carbamate (190, 230 mg, 387.65 μmol, 91.23% yield) as an off white solid. LCMS (ES+): m/z 561 [M+H]⁺

Step 2: To a stirred solution of tert-butyl N-[4-[[4-(4-anilino-3-carbamoyl-6-quinolyl)piperidine-1-carbonyl]amino]butyl]carbamate (190, 230 mg, 410.21 μmol) in DCM (5.00 mL) was added Trifluoroacetic acid (2.96 g, 25.96 mmol, 2.0 mL) and stirred for 1 hr at 25° C. The resulting mixture was concentrated completely under reduced pressure to yield 6-[1-(4-aminobutylcarbamoyl)-4-piperidyl]-4-anilino-quinoline-3-carboxamide (191, 180 mg, 372.84 μmol, 90.89% yield) as an yellow solid. LCMS (ES+): m/z 461 [M+H]⁺

Step 3: To a stirred solution of 2-[2-(2,6-dioxo-3-piperidyl)-1,3-dioxo-isoindolin-4-yl]oxyacetic acid (152, 80 mg, 240.77 μmol) and 6-[1-(4-aminobutylcarbamoyl)-4-piperidyl]-4-anilino-quinoline-3-carboxamide (191, 121.98 mg, 264.85 μmol) in DMF (10.0 mL) was added DIPEA (155.59 mg, 1.20 mmol, 209.69 uL) and HATU (137.32 mg, 361.16 μmol). The resulting mixture was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[1-[4-[[2-[2-(2,6-dioxo-3-piperidyl)-1,3-dioxo-isoindolin-4-yl]oxyacetyl]amino]butylcarbamoyl]-4-piperidyl]quinoline-3-carboxamide (Compound 335, 30 mg, 35.09 μmol, 14.57% yield) as an yellow solid. LCMS (ES+): m/z 775 [M+H]⁺

### Synthesis of Compound 336

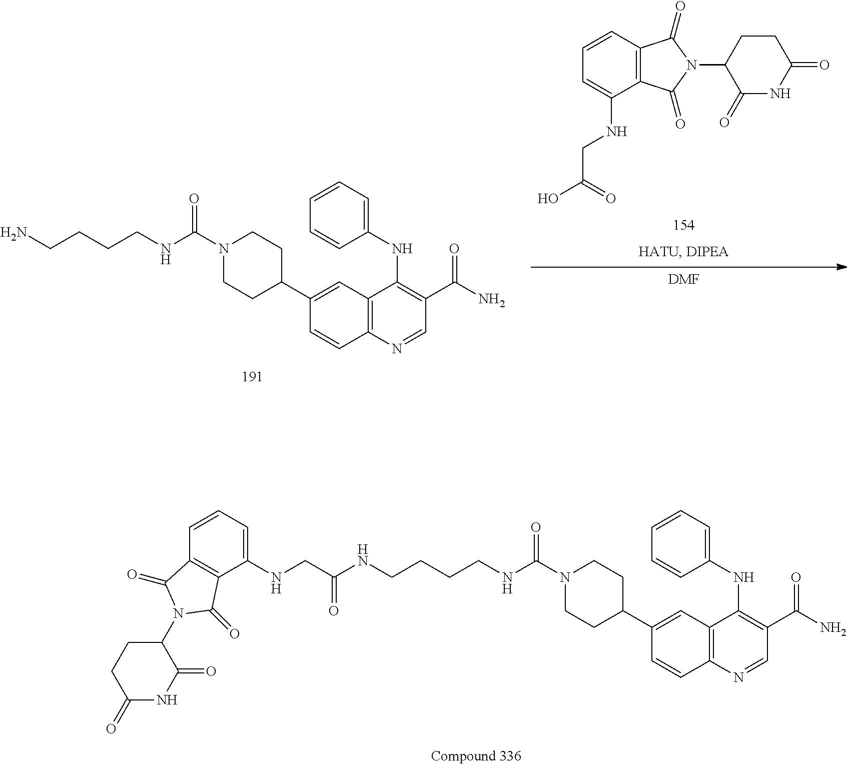

To a stirred solution of 2-[[2-(2,6-dioxo-3-piperidyl)-1,3-dioxo-isoindolin-4-yl]amino]acetic acid (154, 80 mg, 241.49 μmol) and 6-[1-(4-aminobutylcarbamoyl)-4-piperidyl]-4-anilino-quinoline-3-carboxamide (191, 111.22 mg, 241.49 μmol) in DMF (10.0 mL) was added DIPEA (156.05 mg, 1.21 mmol, 210.31 uL) and HATU (137.73 mg, 362.23 μmol). The resulting solution was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[1-[4-[[2-[[2-(2,6-dioxo-3-piperidyl)-1,3-dioxo-isoindolin-4-yl]amino]acetyl]amino]butylcarbamoyl]-4-piperidyl]quinoline-3-carboxamide (Compound 336, 55 mg, 69.17 μmol, 28.64% yield) as an yellow solid. LCMS (ES+): m/z 774 [M+H]⁺

### Synthesis of Compound 337

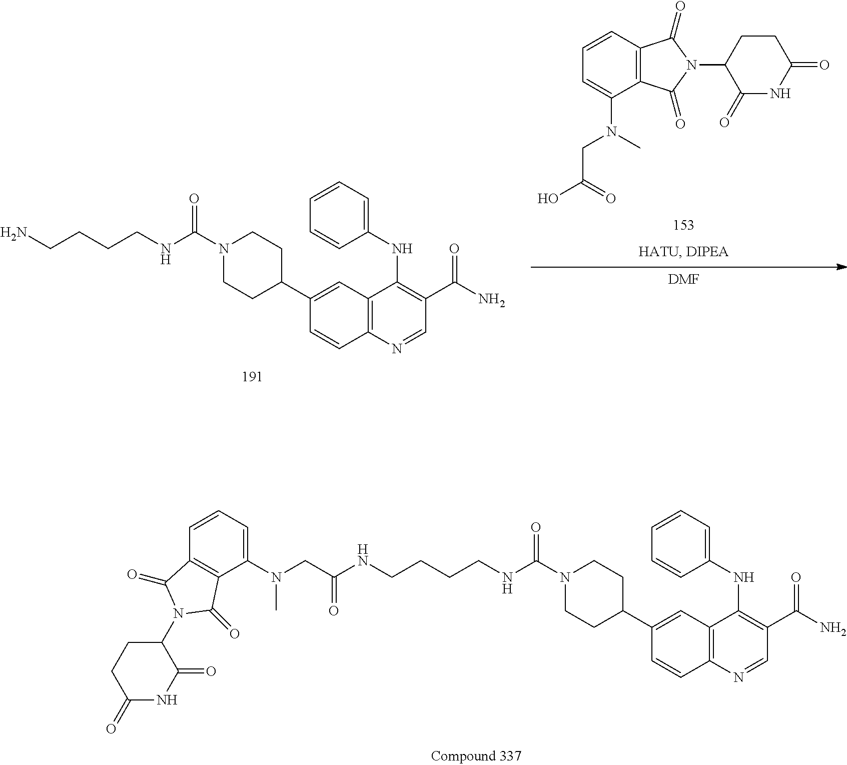

To a stirred solution of 2-[[2-(2,6-dioxo-3-piperidyl)-1,3-dioxo-isoindolin-4-yl]-methyl-amino]acetic acid (153, 80 mg, 231.68 μmol) and 6-[1-(4-aminobutylcarbamoyl)-4-piperidyl]-4-anilino-quinoline-3-carboxamide (191, 106.70 mg, 231.68 μmol) in DMF (10.0 mL) was added DIPEA (149.71 mg, 1.16 mmol, 201.77 uL) and HATU (132.14 mg, 347.52 μmol). The resulting solution was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase prep HPLC to yield 4-anilino-6-[1-[4-[[2-[[2-(2,6-dioxo-3-piperidyl)-1,3-dioxo-isoindolin-4-yl]-methyl-amino]acetyl]amino]butylcarbamoyl]-4-piperidyl]quinoline-3-carboxamide (Compound 337, 25 mg, 30.57 μmol, 13.19% yield) as an yellow solid. LCMS (ES+): m/z 788 [M+H]⁺

### Synthesis of Compound 338

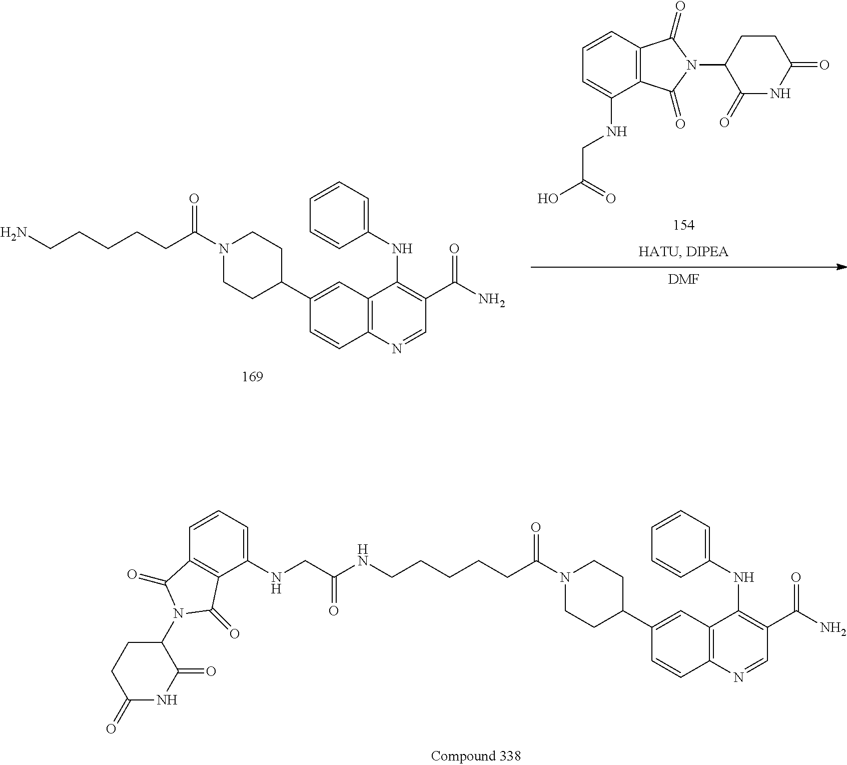

To a stirred solution of 2-[[2-(2,6-dioxo-3-piperidyl)-1,3-dioxo-isoindolin-4-yl]amino]acetic acid (154, 90 mg, 271.67 μmol) and 6-[1-(6-aminohexanoyl)-4-piperidyl]-4-anilino-quinoline-3-carboxamide (169, 124.86 mg, 271.67 μmol) in DMF (10.0 mL) was added DIPEA (175.56 mg, 1.36 mmol, 236.60 uL) and HATU (154.95 mg, 407.51 μmol). The resulting solution was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to 4-anilino-6-[1-[6-[[2-[[2-(2,6-dioxo-3-piperidyl)-1,3-dioxo-isoindolin-4-yl]amino]acetyl]amino]hexanoyl]-4-piperidyl]quinoline-3-carboxamide (Compound 338, 20 mg, 25.26 μmol, 9.30% yield) as an yellow solid. LCMS (ES+): m/z 773 [M+H]⁺

### Synthesis of Compound 339

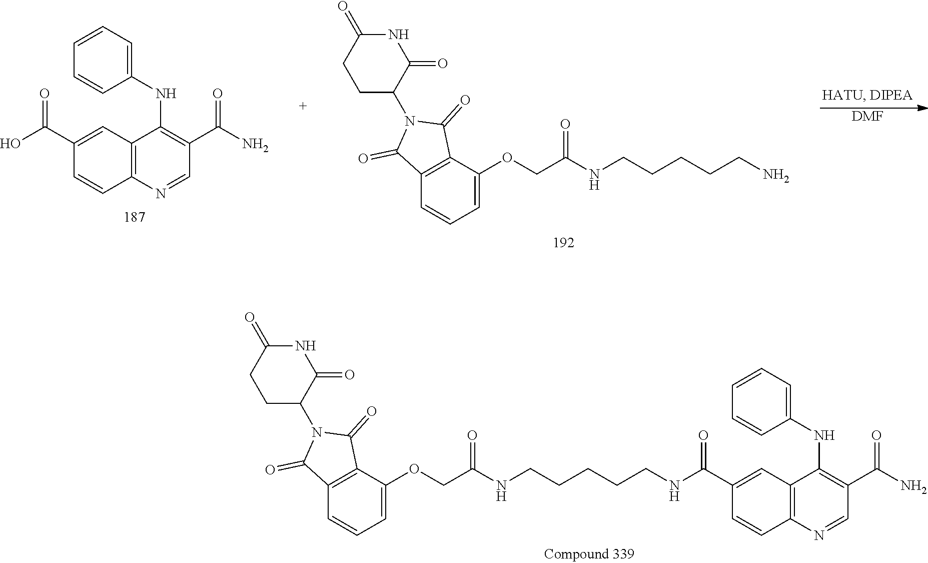

An oven dried round bottom flask was charged with a solution of 4-anilino-3-carbamoyl-quinoline-6-carboxylic acid (187, 30 mg, 97.62 μmol) in DMF (2 mL). N-(5-aminopentyl)-2-[2-(2,6-dioxo-3-piperidyl)-1,3-dioxo-isoindolin-4-yl]oxy-acetamide (192, 48.78 mg, 117.15 μmol), DIPEA (63.09 mg, 488.12 μmol, 85.02 uL) and HATU (44.54 mg, 117.15 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-N6-[5-[[2-[2-(2,6-dioxo-3-piperidyl)-1,3-dioxo-isoindolin-4-yl]oxyacetyl]amino]pentyl]quinoline-3,6-dicarboxamide (Compound 339, 17.82 mg, 21.73 μmol, 22.26% yield) as yellow solid. LCMS (ES+): m/z 706 [M+H]⁺

### Synthesis of Compound 340

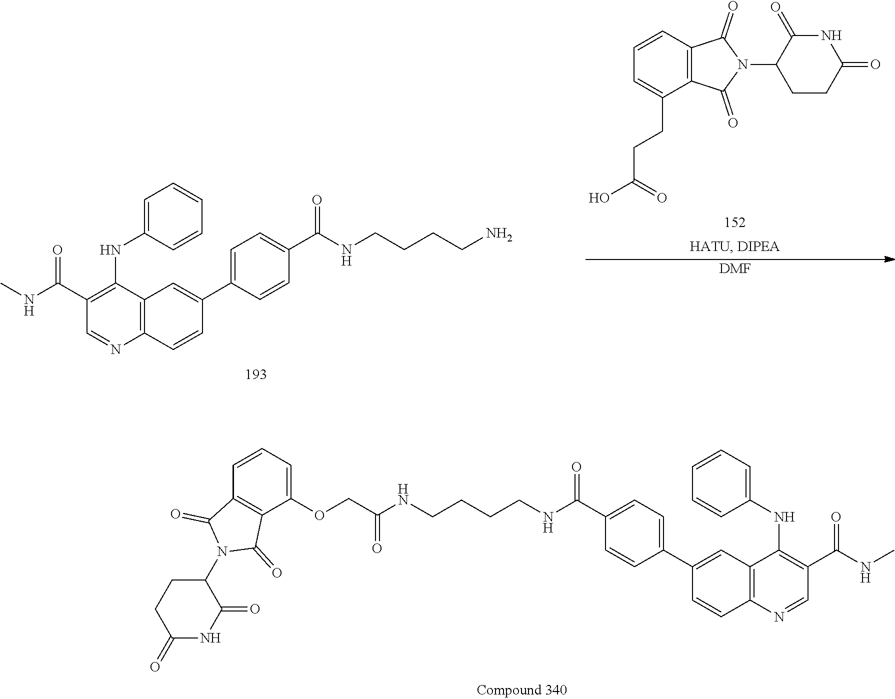

To a stirred solution of 2-[2-(2,6-dioxo-3-piperidyl)-1,3-dioxo-isoindolin-4-yl]oxyacetic acid (152, 60 mg, 180.58 μmol) and 6-[4-(4-aminobutylcarbamoyl)phenyl]-4-anilino-N-methyl-quinoline-3-carboxamide (193, 92.87 mg, 198.64 μmol) in DMF (5.0 mL) was added DIPEA (116.69 mg, 902.90 μmol, 157.27 uL) and HATU (102.99 mg, 270.87 μmol) and stirred for 16 hr at 25° C. The reaction mixture was diluted with water (20 mL) and extracted with ethyl acetate (3×15 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[4-[4-[[2-[2-(2,6-dioxo-3-piperidyl)-1,3-dioxo-isoindolin-4-yl]oxyacetyl]amino]butylcarbamoyl]phenyl]-N-methyl-quinoline-3-carboxamide (Compound 340, 60 mg, 72.92 μmol, 40.38% yield) as an yellow solid. H NMR (400 MHz, DMSO-d₆) δ 11.32 (s, 1H), 11.11 (s, 1H), 8.83 (s, 1H), 8.77 (s, 1H), 8.58 (t, J=5.6 Hz, 1H), 8.53-8.46 (m, 1H), 8.41 (dd, J=8.8, 1.8 Hz, 1H), 8.08 (d, J=8.8 Hz, 1H), 8.03-7.96 (m, 3H), 7.85-7.77 (m, 3H), 7.50-7.43 (m, 3H), 7.39 (d, J=8.5 Hz, 1H), 7.37-7.32 (m, 1H), 7.32-7.27 (m, 2H), 5.11 (dd, J=12.9, 5.4 Hz, 1H), 4.78 (s, 2H), 3.29 (q, J=6.3 Hz, 2H), 3.20 (q, J=6.5 Hz, 2H), 2.88 (ddd, J=17.3, 13.9, 5.4 Hz, 1H), 2.62-2.53 (m, 2H), 2.35 (d, J=4.5 Hz, 3H), 2.07-1.98 (m, 1H), 1.61-1.44 (m, 4H). LCMS (ES+): m/z 782 [M+H]⁺

### Synthesis of Compound 341

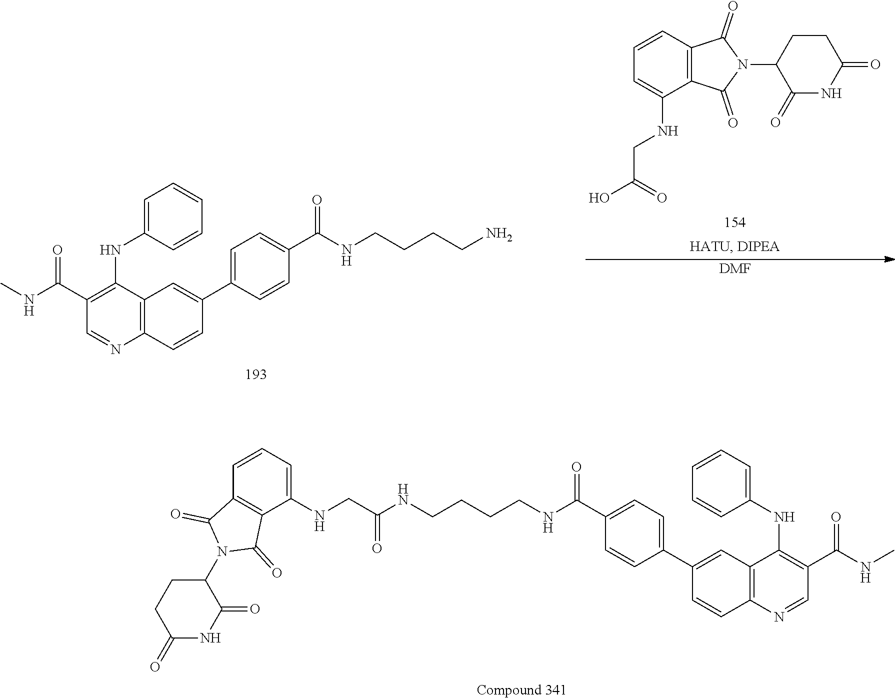

To a stirred solution of 2-[[2-(2,6-dioxo-3-piperidyl)-1,3-dioxo-isoindolin-4-yl]amino]acetic acid (154, 21.26 mg, 64.16 μmol) and 6-[4-(4-aminobutylcarbamoyl)phenyl]-4-anilino-N-methyl-quinoline-3-carboxamide (193, 30 mg, 64.16 μmol) in DMF (5.0 mL) was added DIPEA (41.46 mg, 320.81 μmol, 55.88 uL) and HATU (36.59 mg, 96.24 μmol). The resulting solution was stirred for 16 hr at 25° C. The reaction mixture was diluted with water (20 mL) and extracted with ethyl acetate (3×20 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[4-[4-[[2-[2-(2,6-dioxo-3-piperidyl)-1,3-dioxo-isoindolin-4-yl]oxyacetyl]amino]butylcarbamoyl]phenyl]-N-methyl-quinoline-3-carboxamide (Compound 341, 15 mg, 18.91 μmol, 29.47% yield) as an yellow solid. ¹H NMR (400 MHz, DMSO-d₆) δ 11.25 (s, 1H), 11.12 (s, 1H), 8.82 (s, 1H), 8.75 (s, 1H), 8.60 (s, 1H), 8.51 (s, 1H), 8.40 (d, J=8.9 Hz, 1H), 8.16 (s, 1H), 8.08 (d, J=8.6 Hz, 1H), 7.99 (d, J=8.0 Hz, 2H), 7.82 (d, J=8.1 Hz, 2H), 7.59 (t, J=7.9 Hz, 1H), 7.46 (t, J=7.9 Hz, 2H), 7.38-7.25 (m, 3H), 7.08-6.92 (m, 2H), 6.86 (d, J=8.6 Hz, 1H), 5.11-5.03 (m, 1H), 3.93 (d, J=5.3 Hz, 1H), 3.21-3.09 (m, 2H), 2.89 (s, 1H), 2.63-2.55 (m, 2H), 2.37 (s, 3H), 2.05-1.96 (m, 1H), 1.59-1.41 (m, 4H). LCMS (ES+): m/z 783 [M+H]⁺

### Synthesis of Compound 342

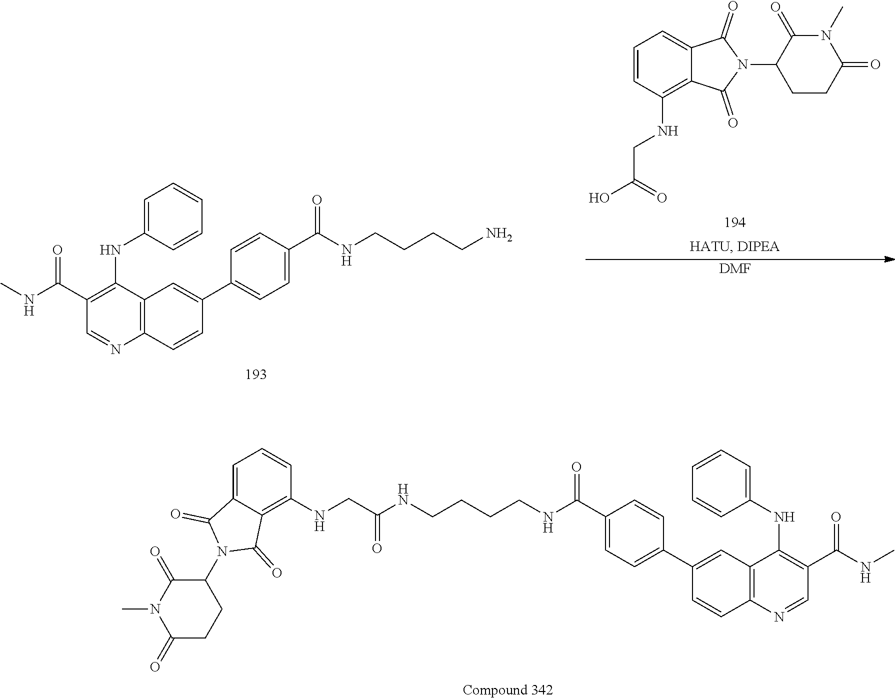

To a stirred solution of 6-(4-((4-aminobutyl)carbamoyl)phenyl)-N-methyl-4-(phenylamino)quinoline-3-carboxamide (193, 64.5 mg, 138 μmol) in DMF (5.00 mL) was added 2-((2-(1-methyl-2,6-dioxopiperidin-3-yl)-1,3-dioxoisoindolin-4-yl)amino)acetic acid (194, 40 mg, 115 HATU (85.5 mg, 225 μmol) and N,N-Diisopropylethylamine (96.8 mg, 749 μmol) at room temperature and stirred over night at room temperature. Added water (25 ml) to the reaction mixture and extracted with ethyl acetate (3×20 mL). The combined organic extracts were washed with water and brine solution, dried over anhydrous sodium sulfate, filtered and evaporated the solvent completely under reduced pressure. The resulting crude was purified by Reverse phase preparative HPLC to yield N-methyl-6-(4-((4-(2-((2-(1-methyl-2,6-dioxopiperidin-3-yl)-1,3-dioxoisoindolin-4-yl)amino)acetamido)butyl)carbamoyl)phenyl)-4-(phenylamino)quinoline-3-carboxamide (Compound 342, 15.3 mg, 19.3 μmol, 16.7%) as a yellow colored solid. LCMS (ES+): m/z 795 [M+H]⁺

### Synthesis of Compound 343

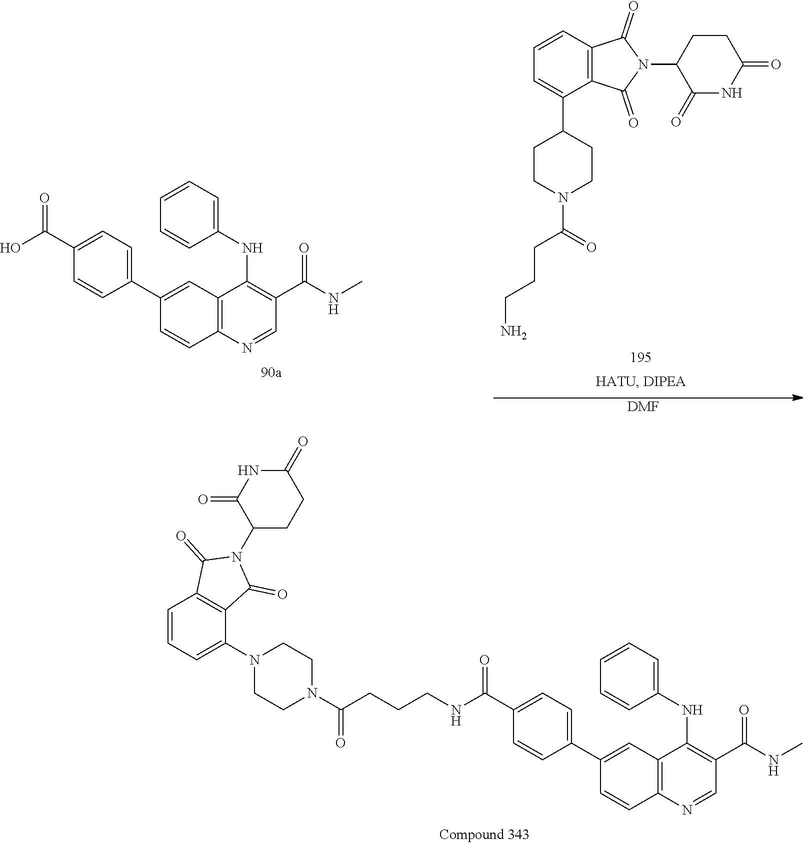

To an oven dried round bottom flask charged with a solution of 4-[4-anilino-3-(methylcarbamoyl)-6-quinolyl]benzoic acid (90a, 80 mg, 201.30 μmol) in DMF (3 mL), 4-[4-(4-aminobutanoyl)piperazin-1-yl]-2-(2,6-dioxo-3-piperidyl)isoindoline-1,3-dione (195, 103.26 mg, 241.56 μmol), N,N-Diisopropylethylamine (130.08 mg, 1.01 mmol, 175.31 uL) and HATU (91.85 mg, 241.56 μmol) were added. The reaction mixture was stirred for 16 h at room temperature. The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[4-[[4-[4-[2-(2,6-dioxo-3-piperidyl)-1,3-dioxo-isoindolin-4-yl]piperazin-1-yl]-4-oxo-butyl]carbamoyl]phenyl]-N-methyl-quinoline-3-carboxamide (Compound 343, 26.37 mg, 30.53 μmol, 15.17% yield) as yellow solid. LCMS (ES+): m/z 807 [M+H]⁺

### Synthesis of Compound 344

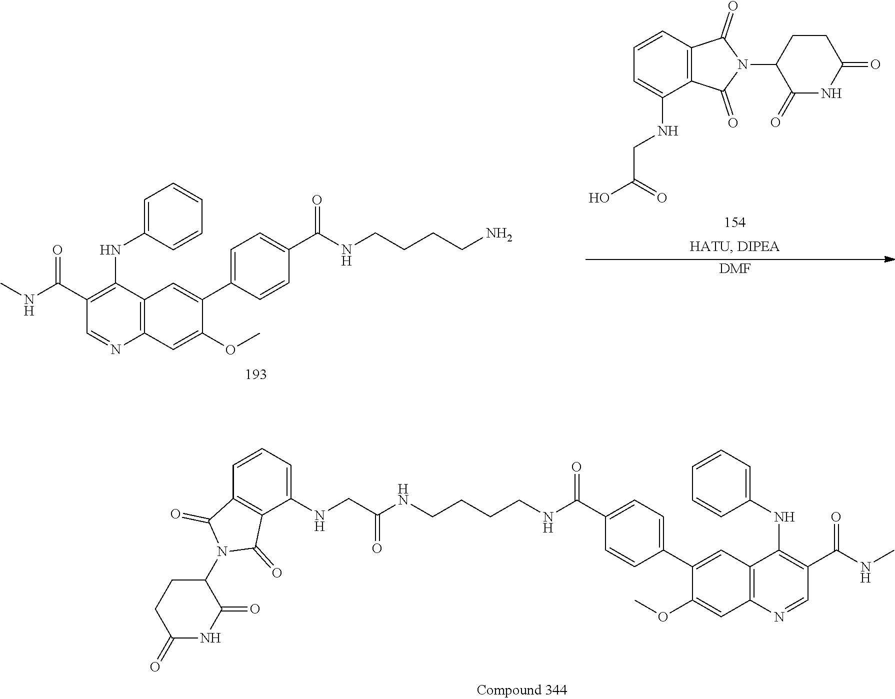

To a stirred solution of 2-[[2-(2,6-dioxo-3-piperidyl)-1,3-dioxo-isoindolin-4-yl]amino]acetic acid (154, 100 mg, 301.86 μmol) and 6-[4-(4-aminobutylcarbamoyl)phenyl]-4-anilino-7-methoxy-N-methyl-quinoline-3-carboxamide (193, 150.20 mg, 301.86 μmol) in DMF (10 mL) was added DIPEA (195.07 mg, 1.51 mmol, 262.89 uL) and HATU (172.16 mg, 452.79 μmol). The resulting mixture was stirred for 16 hr at 25° C. The resulting mixture was diluted with water (30 mL) and extracted with ethyl acetate (3×20 mL). The combined organic extracts were washed with water and brine solution, dried over anhydrous sodium sulfate, filtered and evaporated the solvent completely under reduced pressure. The resulting crude was purified by reverse phase preparative HPLC to yield 6-(4-((4-(2-((2-(2,6-dioxopiperidin-3-yl)-1,3-dioxoisoindolin-4-yl)amino)acetamido)butyl)carbamoyl)phenyl)-7-methoxy-N-methyl-4-(phenylamino)quinoline-3-carboxamide (Compound 344, 25 mg, 29.46 μmol, 9.76% yield) as an yellow solid. ¹H NMR (400 MHz, DMSO-d₆) δ 11.28 (s, 1H), 11.10 (s, 1H), 8.79 (s, 1H), 8.61-8.49 (m, 2H), 8.23 (s, 1H), 8.13 (t, J=5.8 Hz, 1H), 7.91-7.85 (m, 2H), 7.61-7.54 (m, 1H), 7.50-7.39 (m, 5H), 7.31 (t, J=7.4 Hz, 1H), 7.25 (d, J=7.9 Hz, 2H), 7.05 (dd, J=7.1, 1.7 Hz, 1H), 6.95 (t, J=5.8 Hz, 1H), 6.85 (d, J=8.5 Hz, 1H), 5.07 (dd, J=12.9, 5.3 Hz, 1H), 3.98 (s, 3H), 3.92 (d, J=5.4 Hz, 2H), 3.27 (q, J=6.1 Hz, 2H), 3.14 (q, J=6.5 Hz, 2H), 2.94-2.82 (m, 1H), 2.62-2.53 (m, 2H), 2.43 (d, J=4.6 Hz, 3H), 2.06-1.96 (m, 1H), 1.57-1.41 (m, 4H). LCMS (ES+): m/z 811 [M+H]⁺

### Synthesis of Compound 345

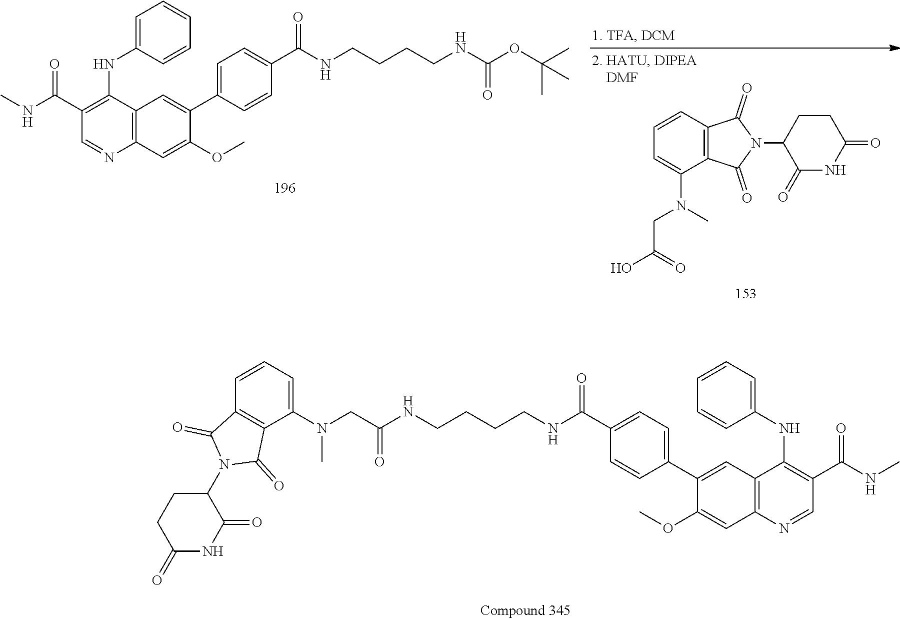 [SMILES: CNC(=O)c1cnc2cc(OC)c(-c3ccc(C(=O)NCCCCNC(=O)OC(C)(C)C)cc3)cc2c1Nc1ccccc1 ; CN(CC(=O)O)c1cccc2c1C(=O)N(C1CCC(=O)NC1=O)C2=O ; CNC(=O)c1cnc2cc(OC)c(-c3ccc(C(=O)NCCCCNC(=O)CN(C)c4cccc5c4C(=O)N(C4CCC(=O)NC4=O)C5=O)cc3)cc2c1Nc1ccccc1]

An oven dried round bottom flask was charged with a solution of tert-butyl N-[4-[[4-[4-anilino-7-methoxy-3-(methylcarbamoyl)-6-quinolyl]benzoyl]amino]butyl]carbamate (196, 124.63 mg, 208.51 μmol) in Dichloromethane (5 mL), Trifluoroacetic acid (4.44 g, 38.94 mmol, 3 mL) was added at room temperature. The reaction mixture was stirred for an hour at room temperature and the reaction mixture was concentrated under reduced pressure. To a solution of crude product in DMF (4 mL), 2-[[2-(2,6-dioxo-3-piperidyl)-1,3-dioxo-isoindolin-4-yl]-methyl-amino]acetic acid (153, 60 mg, 173.76 μmol), DIPEA (112.28 mg, 868.79 μmol, 151.32 uL) and HATU (99.10 mg, 260.64 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 6-(4-((4-(2-((2-(2,6-dioxopiperidin-3-yl)-1,3-dioxoisoindolin-4-yl)(methyl)amino)acetamido)butyl)carbamoyl)phenyl)-7-methoxy-N-methyl-4-(phenylamino)quinoline-3-carboxamide (Compound 345, 50 mg, 58.92 μmol, 33.91% yield) as yellow solid. ¹H NMR (400 MHz, DMSO-d₆) δ 11.33 (s, 1H), 11.08 (s, 1H), 8.79 (s, 1H), 8.59-8.49 (m, 2H), 8.26 (s, 1H), 7.96 (t, J=5.6 Hz, 1H), 7.91-7.86 (m, 2H), 7.62 (dd, J=8.5, 6.9 Hz, 1H), 7.46 (dt, J=14.4, 7.7 Hz, 5H), 7.33 (t, J=7.4 Hz, 1H), 7.29-7.21 (m, 4H), 5.08 (dd, J=12.9, 5.4 Hz, 1H), 4.18 (d, J=17.0 Hz, 1H), 4.13 (d, J=7.6 Hz, 1H), 3.99 (s, 3H), 3.27 (dd, J=11.4, 4.9 Hz, 2H), 3.11 (q, J=5.9 Hz, 2H), 3.01 (s, 4H), 2.87 (td, J=16.9, 15.5, 5.4 Hz, 1H), 2.61-2.54 (m, 2H), 2.42 (d, J=4.6 Hz, 3H), 2.05-1.96 (m, 1H), 1.58-1.42 (m, 4H). LCMS (ES+): m/z 825 [M+H]⁺

### Synthesis of Compound 346

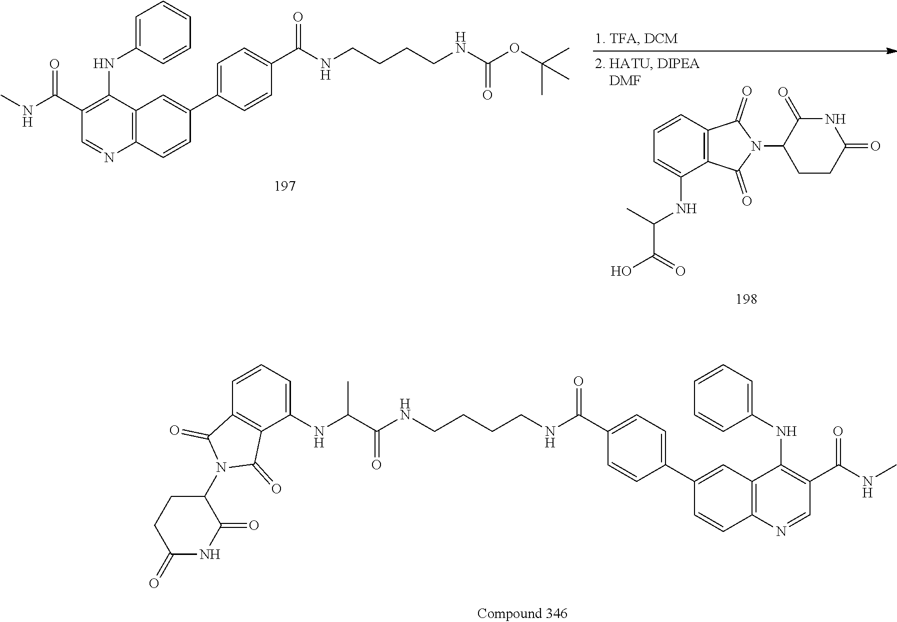 [SMILES: CNC(=O)c1cnc2ccc(-c3ccc(C(=O)NCCCCNC(=O)OC(C)(C)C)cc3)cc2c1Nc1ccccc1 ; CC(Nc1cccc2c1C(=O)N(C1CCC(=O)NC1=O)C2=O)C(=O)O ; CNC(=O)c1cnc2ccc(-c3ccc(C(=O)NCCCCNC(=O)C(C)Nc4cccc5c4C(=O)N(C4CCC(=O)NC4=O)C5=O)cc3)cc2c1Nc1ccccc1]

An oven dried round bottom flask was charged with a solution of tert-butyl N-[4-[[4-[4-anilino-3-(methylcarbamoyl)-6-quinolyl]benzoyl]amino]butyl]carbamate (197, 60 mg, 105.69 μmol) in Dichloromethane (3 mL), Trifluoroacetic acid (1.48 g, 12.98 mmol, 1 mL) was added at room temperature. The reaction mixture was stirred for 2 h at room temperature and the reaction mixture was concentrated under reduced pressure. To a solution of crude product in DMF (3 mL), 2-[[2-(2,6-dioxo-3-piperidyl)-1,3-dioxo-isoindolin-4-yl]amino]propanoic acid (198, 43.79 mg, 126.83 μmol), DIPEA (68.30 mg, 528.45 μmol, 92.05 uL) and HATU (48.23 mg, 126.83 μmol) were added. The reaction mixture was stirred for 16 h at room temperature and the reaction mixture was quenched with water (2 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[4-[4-[2-[[2-(2,6-dioxo-3-piperidyl)-1,3-dioxo-isoindolin-4-yl]amino]propanoylamino]butylcarbamoyl]phenyl]-N-methyl-quinoline-3-carboxamide (Compound 346, 23.17 mg, 25.40 μmol, 24.03% yield) as yellow solid. ¹H NMR (400 MHz, DMSO-d₆) δ 11.11 (s, 1H), 10.52 (s, 1H), 8.82 (s, 1H), 8.57-8.50 (m, 2H), 8.41 (s, 1H), 8.26-8.15 (m, 2H), 8.06-8.00 (m, 1H), 7.91 (d, J=7.6 Hz, 2H), 7.67 (d, J=8.0 Hz, 2H), 7.63-7.56 (m, 1H), 7.36 (t, J=7.8 Hz, 2H), 7.22-7.11 (m, 3H), 7.09-7.03 (m, 1H), 6.92 (d, J=8.6 Hz, 1H), 5.12-5.01 (m, 1H), 4.23-4.13 (m, 1H), 3.29-3.23 (m, 2H), 3.18-3.05 (m, 2H), 3.03-2.95 (m, 1H), 2.93-2.82 (m, 2H), 2.68-2.55 (m, 2H), 2.08-1.97 (m, 1H), 1.48 (s, 3H), 1.40-1.33 (m, 4H). LCMS (ES+): m/z 795 [M+H]⁺

### Synthesis of Compound 347

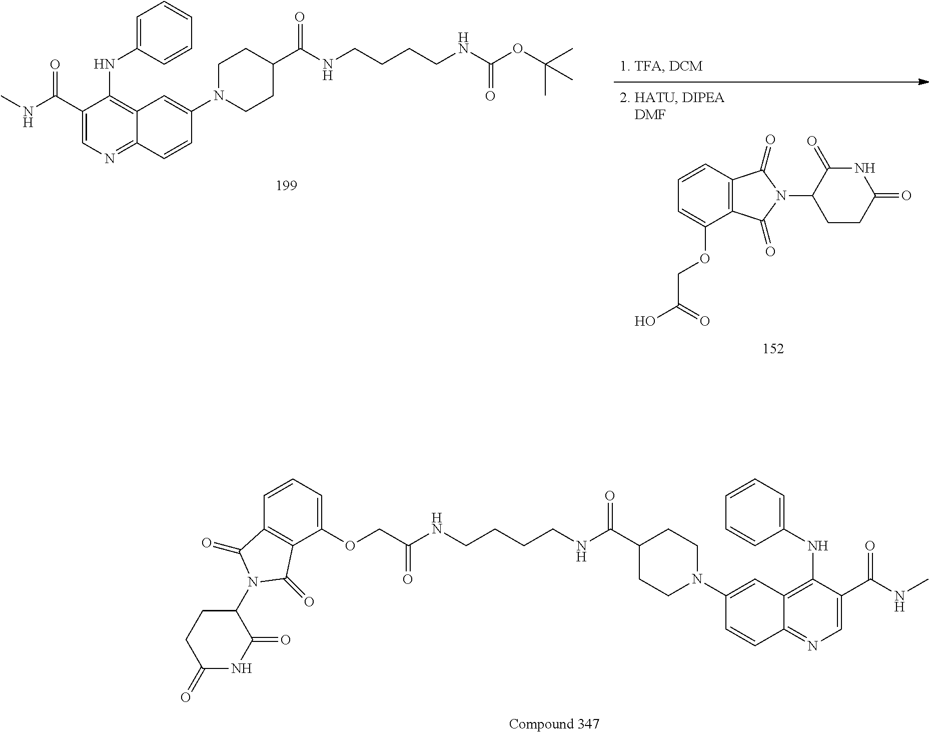 [SMILES: CNC(=O)c1cnc2ccc(N3CCC(C(=O)NCCCCNC(=O)OC(C)(C)C)CC3)cc2c1Nc1ccccc1 ; O=C(O)COc1cccc2c1C(=O)N(C1CCC(=O)NC1=O)C2=O ; CNC(=O)c1cnc2ccc(N3CCC(C(=O)NCCCCNC(=O)COc4cccc5c4C(=O)N(C4CCC(=O)NC4=O)C5=O)CC3)cc2c1Nc1ccccc1]

Into a 25 mL single-necked round-bottomed flask containing a well-stirred solution of tert-butyl N-[4-[[1-[4-anilino-3-(methylcarbamoyl)-6-quinolyl]piperidine-4-carbonyl]amino]butyl]carbamate (199, 150 mg, 261.00 μmol) in anhydrous DCM (4 mL) was added TFA (148.80 mg, 1.31 mmol, 100.54 uL) under nitrogen atmosphere at room temperature, the resulting mixture was stirred at room temperature for 30 minutes. The reaction mixture was concentrated under reduced pressure to afford a crude residue which was dissolved in DMF (3 mL). DIPEA (101.20 mg, 783.00 μmol, 136.38 uL), 2-[2-(2,6-dioxo-3-piperidyl)-1,3-dioxo-isoindolin-4-yl]oxyacetic acid (152, 86.72 mg, 261.00 μmol) and HATU (148.86 mg, 391.50 μmol) were added under nitrogen atmosphere at room temperature. The resulting mixture was stirred at room temperature for 16 h. Ice cold water (15 mL) was added and stirred for 10 minutes, the resulting solid was filtered and dried to afford a crude solid which was purified by prep-HPLC (SUNFIRE OBD C18(100×30)MM 5μ) Mobile phase: A: 0.1% TFA in water B: ACN to afford 4-anilino-6-[4-[4-[[2-[2-(2,6-dioxo-3-piperidyl)-1,3-dioxo-isoindolin-4-yl]oxyacetyl]amino]butylcarbamoyl]-1-piperidyl]-N-methyl-quinoline-3-carboxamide (Compound 347, 50 mg, 61.80 μmol, 23.68% yield) as a yellow solid. ¹H NMR (400 MHz, DMSO-d₆) δ 11.12 (s, 1H), 10.84 (s, 1H), 8.59 (s, 1H), 8.44-8.34 (m, 1H), 8.01-7.93 (m, 1H), 7.82 (d, J=6.5 Hz, 4H), 7.53-7.47 (m, 2H), 7.40 (q, J=8.5, 8.1 Hz, 3H), 7.31-7.20 (m, 3H), 5.12 (dd, J=12.8, 4.6 Hz, 1H), 4.77 (s, 2H), 3.90-3.82 (m, 2H), 3.18-3.10 (m, 2H), 3.03 (s, 2H), 2.91-2.78 (m, 4H), 2.63-2.56 (m, 2H), 2.35-2.29 (m, 4H), 2.07-1.97 (m, 1H), 1.72 (d, J=14.2 Hz, 2H), 1.65-1.56 (m, 2H), 1.41 (s, 4H), 1.23 (s, 1H). LCMS (ES+): m/z 789 [M+H]⁺

### Synthesis of Compound 348

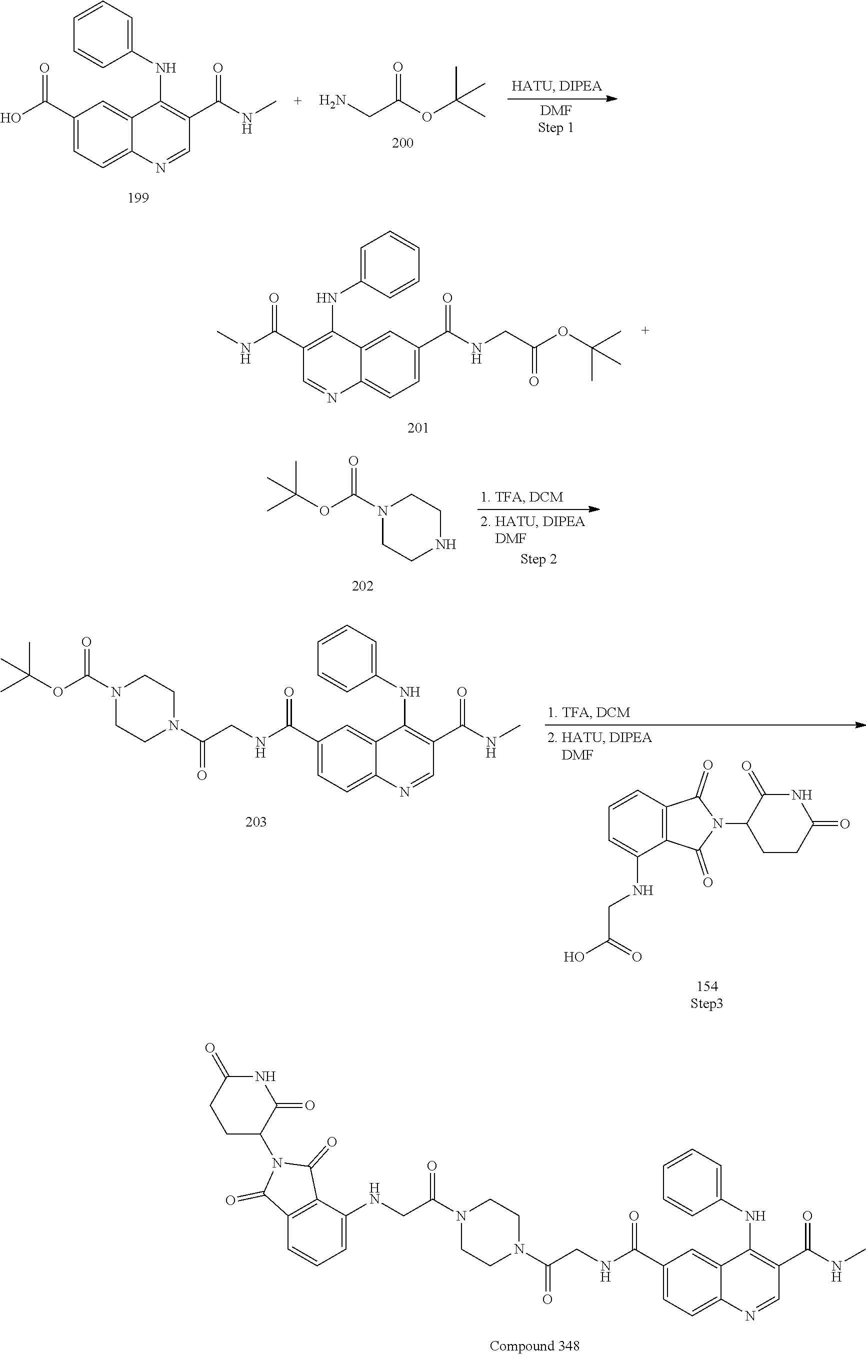

Step 1: Into a 25 mL single-necked round-bottomed flask containing a well-stirred solution of 4-anilino-3-(methylcarbamoyl)quinoline-6-carboxylic acid (199, 70 mg, 217.84 μmol) and tert-butyl 2-aminoacetate (200, 37.15 mg, 283.20 μmol) in DMF (3 mL) were added DIPEA (56.31 mg, 435.69 μmol, 75.89 uL) and HATU (124.25 mg, 326.77 μmol) under nitrogen atmosphere at room temperature, the resulting mixture was stirred at room temperature for 16 h. To the crude mixture, 20 mL of cold water was added and stirred for 10 minutes. The resulting solid was filtered and dried to afford tert-butyl 24-[4-anilino-3-(methylcarbamoyl)quinoline-6-carbonyl]amino]acetate (201, 60 mg, 138.09 μmol, 63.39% yield) as a brown solid. LCMS (ES+): m/z 435 [M+H]⁺

Step 2: Into a 25 mL single-necked round-bottomed flask containing a well-stirred solution of tert-butyl 2-[[4-anilino-3-(methylcarbamoyl)quinoline-6-carbonyl]amino]acetate (201, 60 mg, 138.09 μmol) in anhydrous DCM (4 mL) was added TFA (78.73 mg, 690.47 μmol, 53.20 uL) under nitrogen atmosphere at room temperature, the resulting mixture was stirred at room temperature for 30 minutes. The reaction mixture was concentrated under reduced pressure to afford a crude residue which was dissolved in DMF (3 mL). 1-Boc-piperazine (202, 30.86 mg, 165.71 μmol), DIPEA (53.54 mg, 414.28 μmol, 72.16 uL) and HATU (78.76 mg, 207.14 μmol) were added under nitrogen atmosphere at room temperature. The resulting mixture was stirred at room temperature for 16 h. Ice cold water (15 mL) was added and stirred for 10 minutes, aqueous phase was extracted twice with EtOAc (2×25 mL). The organic layer was dried (anhydrous Na₂SO₄), filtered and the filtrate was concentrated under reduced pressure to afford tert-butyl 4-[2-[[4-anilino-3-(methylcarbamoyl)quinoline-6-carbonyl]amino]acetyl]piperazine-1-carboxylate (203, 60 mg, 109.77 μmol, 79.49% yield) as a crude. LCMS (ES+): m/z 547 [M+H]⁺

Step 3: Into a 25 mL single-necked round-bottomed flask containing a well-stirred solution of tert-butyl 4-[2-[[4-anilino-3-(methylcarbamoyl)quinoline-6-carbonyl]amino]acetyl]piperazine-1-carboxylate (203, 60 mg, 109.77 μmol) in anhydrous DCM (4 mL) was added TFA (62.58 mg, 548.83 μmol, 42.28 uL) under nitrogen atmosphere at room temperature, the resulting mixture was stirred at room temperature for 2 h. The reaction mixture was concentrated under reduced pressure to afford a crude residue which was dissolved in DMF (3 mL). DIPEA (42.56 mg, 329.30 μmol, 57.36 uL), 2-[[2-(2,6-dioxo-3-piperidyl)-1,3-dioxo-isoindolin-4-yl]amino]acetic acid (154, 40.00 mg, 120.74 μmol) and HATU (62.60 mg, 164.65 μmol) were added under nitrogen atmosphere at room temperature. The resulting mixture was stirred at room temperature for 16 h. Ice cold water (15 mL) was added and stirred for 10 minutes, aqueous phase was extracted twice with EtOAc (2×25 mL, the organic layer was dried (anhydrous Na₂SO₄), filtered and the filtrate was concentrated under reduced pressure to afford crude residue, which was purified by prep-HPLC(SUNFIRE OBD C18(100×30)MM 5μ) Mobile phase: A: 0.1% TFA in water B: ACN to afford 4-anilino-N6-[2-[4-[2-[[2-(2,6-dioxo-3-piperidyl)-1,3-dioxo-isoindolin-4-yl]amino]acetyl]piperazin-1-yl]-2-oxo-ethyl]-N3-methyl-quinoline-3,6-dicarboxamide (Compound 348, 3 mg, 3.63 μmol, 3.31% yield) as a pale yellow gummy solid. LCMS (ES+): m/z 760 [M+H]⁺

### Synthesis of Compound 349

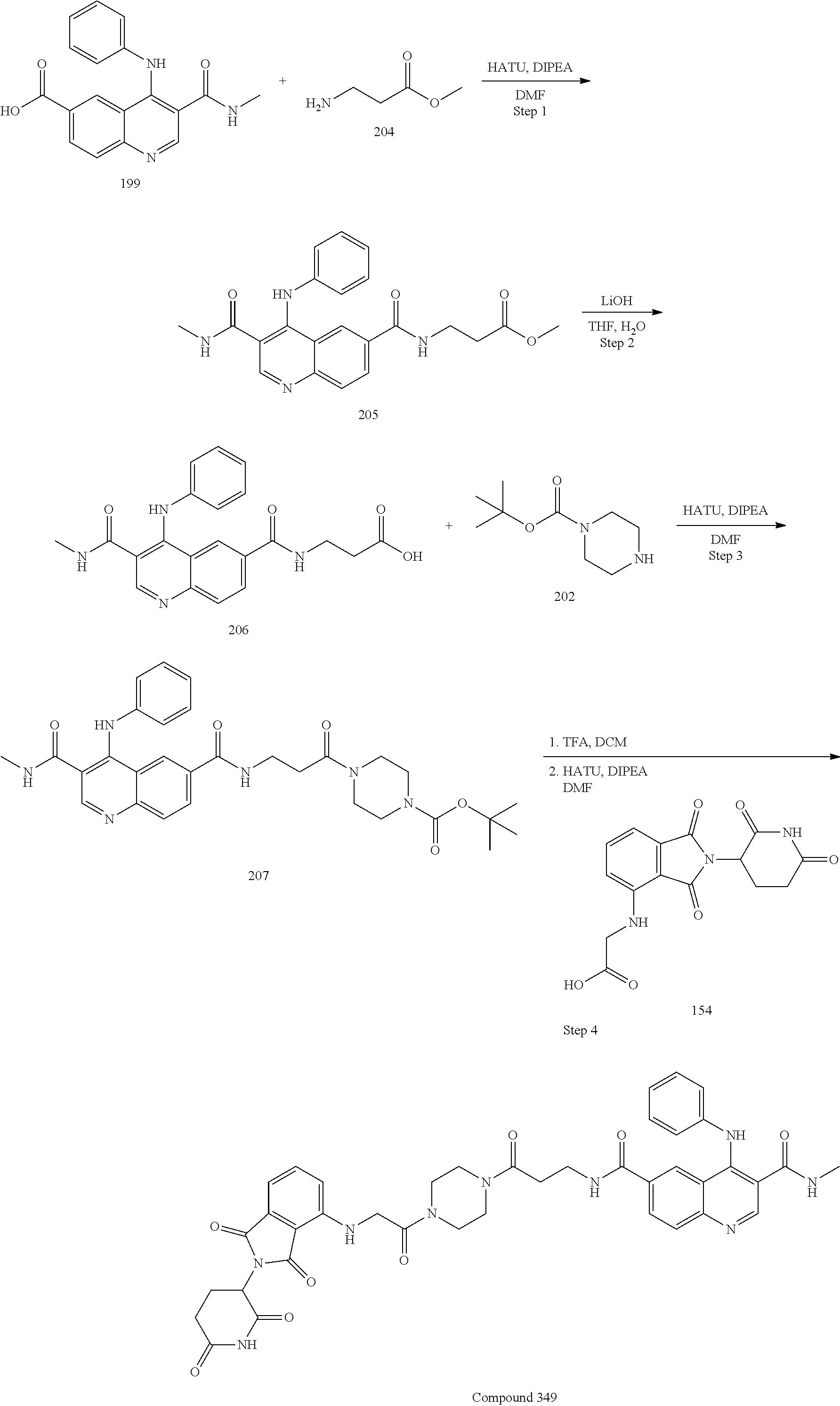

Step 1: Into a 25 mL single-necked round-bottomed flask containing a well-stirred solution of 4-anilino-3-(methylcarbamoyl)quinoline-6-carboxylic acid (199, 150 mg, 466.81 μmol) and methyl 3-aminopropanoate (204, 62.58 mg, 606.85 μmol) in DMF (4 mL) were added DIPEA (120.66 mg, 933.62 μmol, 162.62 uL) and HATU (266.24 mg, 700.21 μmol) under nitrogen atmosphere at room temperature. The resulting mixture was stirred at room temperature for 16 h. To the crude mixture 20 mL of ice cold water was added, aqueous phase was extracted twice with EtOAc (2×50 mL). Combined organic phases were dried (anhydrous Na₂SO₄), filtered and the filtrate was concentrated under reduced pressure to afford methyl 3-[[4-anilino-3-(methylcarbamoyl)quinoline-6-carbonyl]amino]propanoate (205, 150 mg, 369.06 μmol, 79.06% yield) as a crude residue. LCMS (ES+): m/z 407 [M+H]⁺

Step 2: Into a 50 mL single-necked round-bottomed flask containing a well-stirred solution of methyl 34-[4-anilino-3-(methylcarbamoyl)quinoline-6-carbonyl]amino]propanoate (205, 150 mg, 369.06 μmol) in THF (3 mL) was added Lithium hydroxide powder, reagent grade (44.19 mg, 1.85 mmol) in Water (3 mL) at room temperature. The resulting mixture was stirred at room temperature for 16 h. The reaction mixture was concentrated under reduced pressure, to the crude residue 10 mL of water was added and aqueous phase was extracted twice with diethyl ether (2×10 mL) and discarded organic phases. Again aqueous layer was acidified with 1.5N HCl until pH=3 and extracted twice with EtOAc (2×20 mL). Combined organic phases were dried (anhydrous Na₂SO₄), filtered and the filtrate was evaporated under reduced pressure to afford 3-[[4-anilino-3-(methylcarbamoyl)quinoline-6-carbonyl]amino]propanoic acid (206, 50 mg, 127.42 μmol, 34.52% yield) as a brown solid. LCMS (ES+): m/z 393 [M+H]⁺

Step 3: Into a 25 mL single-necked round-bottomed flask containing a well-stirred solution of 3-[[4-anilino-3-(methylcarbamoyl)quinoline-6-carbonyl]amino]propanoic acid (206, 50 mg, 127.42 μmol) in anhydrous DMF (3 mL) were added 1-Boc-piperazine (202, 28.48 mg, 152.90 μmol), DIPEA (32.94 mg, 254.84 μmol, 44.39 uL) and HATU (72.67 mg, 191.13 μmol) under nitrogen atmosphere at room temperature, the resulting mixture was stirred at room temperature for 16 h. Ice cold water (15 mL) was added and stirred for 10 minutes, aqueous phase was extracted twice with EtOAc (2×25 mL) the organic layer was dried (anhydrous Na₂SO₄), filtered and the filtrate was concentrated under reduced pressure to afford tert-butyl 4-[3-[[4-anilino-3-(methylcarbamoyl)quinoline-6-carbonyl]amino]propanoyl]piperazine-1-carboxylate (207, 60 mg, 107.02 μmol, 83.99% yield) as a crude residue. LCMS (ES+): m/z 561 [M+H]⁺

Step 4: Into a 25 mL single-necked round-bottomed flask containing a well-stirred solution of tert-butyl 4-[3-[[4-anilino-3-(methylcarbamoyl)quinoline-6-carbonyl]amino]propanoyl]piperazine-1-carboxylate (207, 60 mg, 107.02 μmol) in anhydrous DCM (4 mL) was added TFA (61.01 mg, 535.10 μmol, 41.23 uL) under nitrogen atmosphere at room temperature, the resulting mixture was stirred at room temperature for 30 minutes. The reaction mixture was concentrated under reduced pressure to afford a crude residue which was dissolved in DMF (4 mL). DIPEA (41.49 mg, 321.06 μmol, 55.92 uL), 2-[[2-(2,6-dioxo-3-piperidyl)-1,3-dioxo-isoindolin-4-yl]amino]acetic acid (154, 39.00 mg, 117.72 μmol) and HATU (61.04 mg, 160.53 μmol) were added under nitrogen atmosphere at room temperature. The resulting mixture was stirred at room temperature for 16 h. Ice cold water (15 mL) was added and stirred for 10 minutes, aqueous phase was extracted twice with EtOAc (2×25 mL, the organic layer was dried (anhydrous Na2SO4), filtered and the filtrate was concentrated under reduced pressure to afford crude residue, which was purified by prep-HPLC(SUNFIRE OBD C18(100×30)MM 5μ) Mobile phase: A: 0.1% TFA in water B: ACN to afford 4-anilino-N6-[3-[4-[2-[[2-(2,6-dioxo-3-piperidyl)-1,3-dioxo-isoindolin-4-yl]amino]acetyl]piperazin-1-yl]-3-oxo-propyl]-N3-methyl-quinoline-3,6-dicarboxamide (Compound 349, 3.5 mg, 4.52 μmol, 4.23% yield) as a yellow solid. LCMS (ES+): m/z 774 [M+H]⁺

### Synthesis of Compound 350

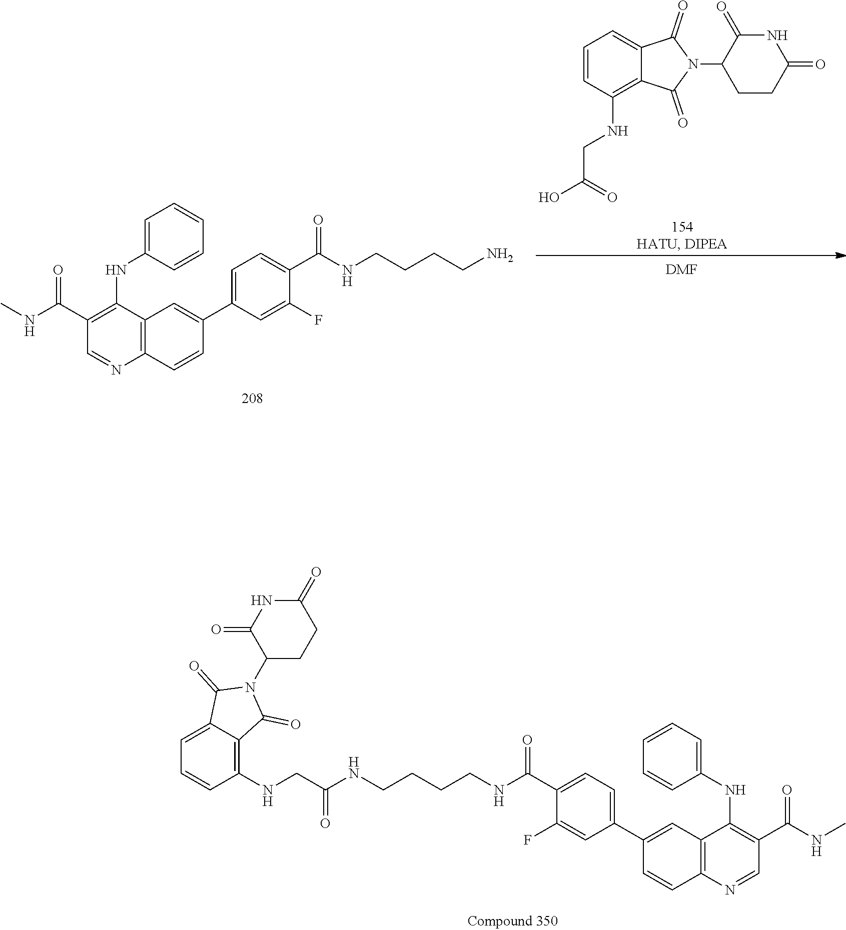

Into a 25 mL single-necked round-bottomed flask containing a well-stirred solution of 6-[4-(4-aminobutylcarbamoyl)-3-fluoro-phenyl]-4-anilino-N-methyl-quinoline-3-carboxamide (208, 73.28 mg, 150.93 μmol) and 2-[[2-(2,6-dioxo-3-piperidyl)-1,3-dioxo-isoindolin-4-yl]amino]acetic acid (154, 50 mg, 150.93 μmol) in DMF (5 mL) were added DIPEA (58.52 mg, 452.79 μmol, 78.87 uL) and HATU (86.08 mg, 226.39 μmol) under nitrogen atmosphere at room temperature, the resulting mixture was stirred at room temperature for 16 h. After completion of reaction, 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 4-anilino-6-[4-[4-[[2-[[2-(2,6-dioxo-3-piperidyl)-1,3-dioxo-isoindolin-4-yl]amino]acetyl]amino]butylcarbamoyl]-3-fluoro-phenyl]-N-methyl-quinoline-3-carboxamide (Compound 350, 85 mg, 106.41 μmol, 70.50% yield). ¹H NMR (400 MHz, DMSO-d₆) δ 11.23 (s, 1H), 11.11 (s, 1H), 8.82 (s, 1H), 8.80 (s, 1H), 8.48 (d, J=4.7 Hz, 1H), 8.45-8.35 (m, 2H), 8.14 (t, J=5.6 Hz, 1H), 8.07 (d, J=8.8 Hz, 1H), 7.79-7.66 (m, 3H), 7.59 (t, J=7.8 Hz, 1H), 7.45 (t, J=7.7 Hz, 2H), 7.34 (t, J=7.6 Hz, 1H), 7.29 (d, J=7.9 Hz, 2H), 7.06 (d, J=7.1 Hz, 1H), 6.95 (d, J=5.9 Hz, 1H), 6.86 (d, J=8.6 Hz, 1H), 5.07 (dd, J=12.9, 5.4 Hz, 1H), 3.93 (d, J=5.2 Hz, 2H), 3.26 (q, J=6.2 Hz, 2H), 3.14 (q, J=6.2 Hz, 2H), 2.89 (ddd, J=18.0, 14.2, 5.4 Hz, 1H), 2.69-2.55 (m, 2H), 2.33 (d, J=4.5 Hz, 3H), 2.08-1.96 (m, 1H), 1.56-1.42 (m, 4H). LCMS (ES+): m/z 799 [M+H]⁺

### Synthesis of Compound 351

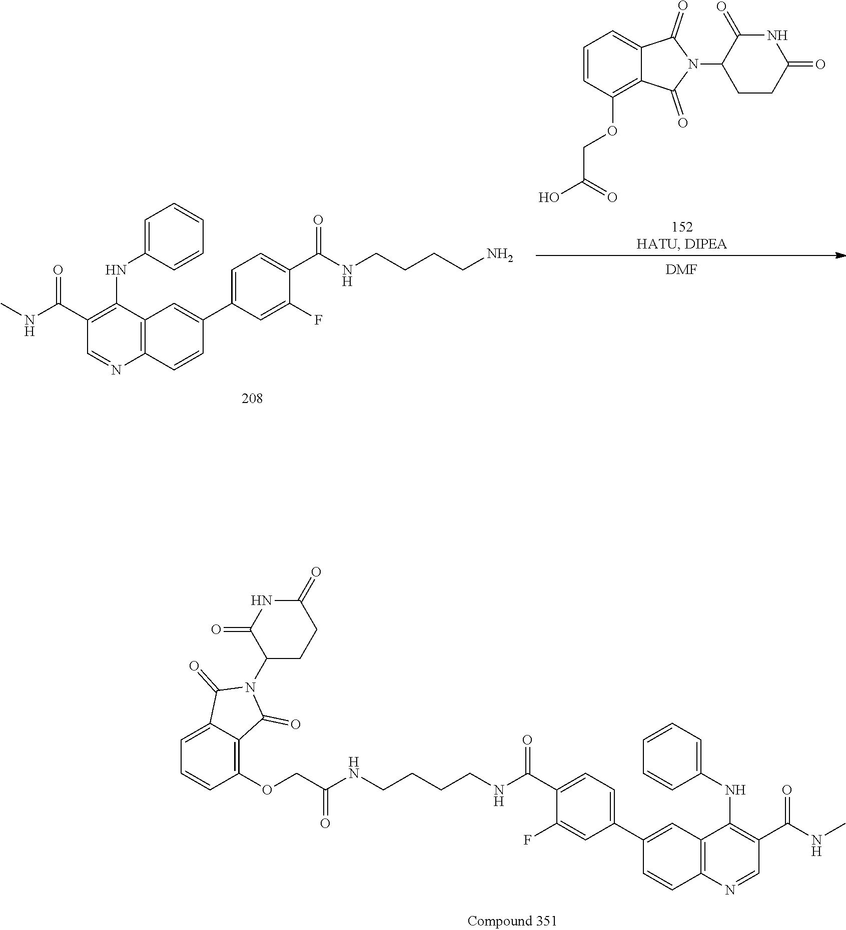

Into a 25 mL single-necked round-bottomed flask containing a well-stirred solution of 6-[4-(4-aminobutylcarbamoyl)-3-fluoro-phenyl]-4-anilino-N-methyl-quinoline-3-carboxamide (208, 73.07 mg, 150.48 μmol) and 2-[2-(2,6-dioxo-3-piperidyl)-1,3-dioxo-isoindolin-4-yl]oxyacetic acid (152, 50 mg, 150.48 μmol) in DMF (5 mL) were added DIPEA (58.35 mg, 451.45 μmol, 78.63 uL) and HATU (85.83 mg, 225.72 μmol) under nitrogen atmosphere at room temperature. The resulting mixture was stirred at room temperature for 16 h. After completion of reaction, 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 4-anilino-6-[4-[4-[[2-[2-(2,6-dioxo-3-piperidyl)-1,3-dioxo-isoindolin-4-yl]oxyacetyl]amino]butylcarbamoyl]-3-fluoro-phenyl]-N-methyl-quinoline-3-carboxamide (Compound 351, 50 mg, 62.52 μmol, 41.54% yield). ¹H NMR (400 MHz, DMSO-d₆) δ 11.29 (s, 1H), 11.13 (s, 1H), 8.83 (s, 2H), 8.52-8.36 (m, 3H), 8.09 (d, J=8.9 Hz, 1H), 8.02 (t, J=5.8 Hz, 1H), 7.82 (t, J=7.9 Hz, 1H), 7.77-7.68 (m, 3H), 7.48 (dd, J=14.2, 7.3 Hz, 3H), 7.40 (d, J=8.5 Hz, 1H), 7.36 (t, J=7.4 Hz, 1H), 7.31 (d, J=7.9 Hz, 2H), 5.13 (dd, J=12.9, 5.3 Hz, 1H), 4.79 (s, 2H), 3.28 (q, J=6.2 Hz, 2H), 3.20 (q, J=6.3 Hz, 2H), 2.89 (ddd, J=18.2, 14.1, 5.4 Hz, 1H), 2.71-2.57 (m, 2H), 2.33 (d, J=4.5 Hz, 3H), 2.09-1.98 (m, 1H), 1.61-1.48 (m, 4H). LCMS (ES+): m/z 800 [M+H]⁺

### Synthesis of Compound 352

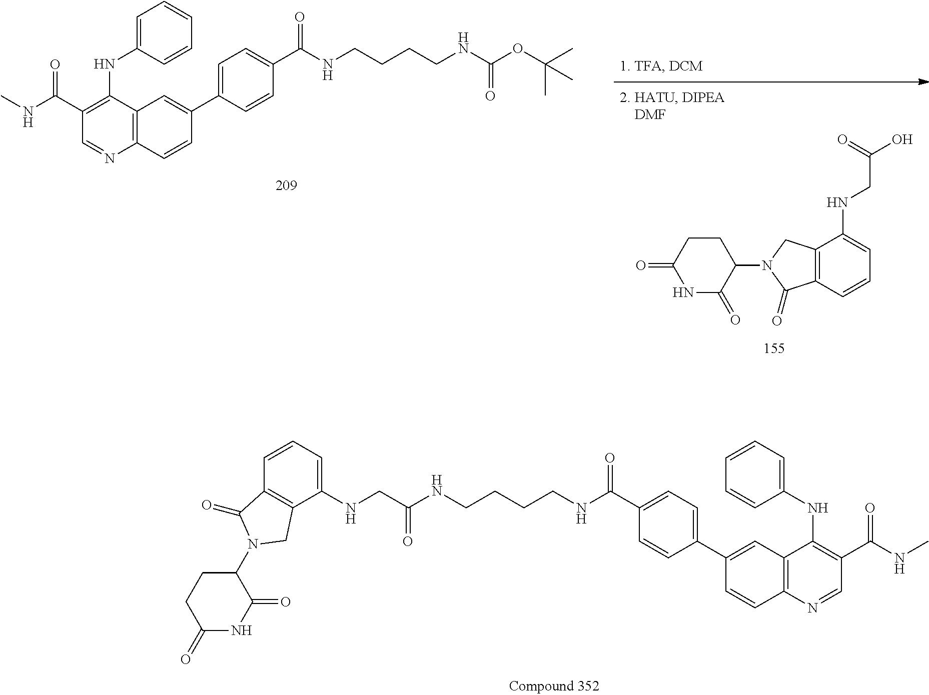 [SMILES: CNC(=O)c1cnc2ccc(-c3ccc(C(=O)NCCCCNC(=O)OC(C)(C)C)cc3)cc2c1Nc1ccccc1 ; O=C(O)CNc1cccc2c1CN(C1CCC(=O)NC1=O)C2=O ; CNC(=O)c1cnc2ccc(-c3ccc(C(=O)NCCCCNC(=O)CNc4cccc5c4CN(C4CCC(=O)NC4=O)C5=O)cc3)cc2c1Nc1ccccc1]

An oven dried round bottom flask was charged with a solution of tert-butyl N-[4-[[4-[4-anilino-3-(methylcarbamoyl)-6-quinolyl]benzoyl]amino]butyl]carbamate (209, 40 mg, 70.46 μmol) in Dichloromethane (3 mL), Trifluoroacetic acid (2.96 g, 25.96 mmol, 2 mL) was added at room temperature. The reaction mixture was stirred for 2 h at room temperature and the reaction mixture was concentrated under reduced pressure. To a solution of crude product in DMF (2 mL), 2-[[2-(2,6-dioxo-3-piperidyl)-1-oxo-isoindolin-4-yl]amino]acetic acid (155, 22.36 mg, 70.46 μmol), DIPEA (45.53 mg, 352.31 μmol, 61.37 uL) and HATU (40.19 mg, 105.69 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[4-[4-[[2-[[2-(2,6-dioxo-3-piperidyl)-1-oxo-isoindolin-4-yl]amino]acetyl]amino]butylcarbamoyl]phenyl]-N-methyl-quinoline-3-carboxamide (Compound 352, 5.38 mg, 6.56 μmol, 9.31% yield) as a yellow solid. LCMS (ES+): m/z 767 [M+H]⁺

### Synthesis of Compound 353

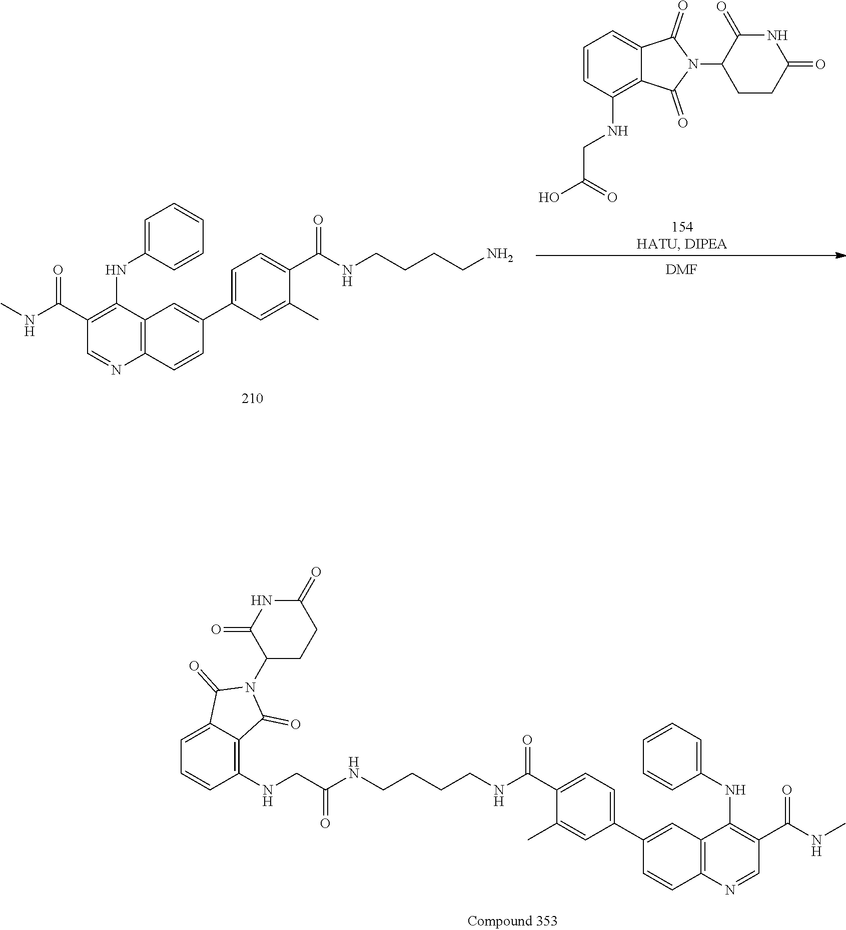

Into a 25 mL single-necked round-bottomed flask containing a well-stirred solution of 6-[4-(4-aminobutylcarbamoyl)-3-methyl-phenyl]-4-anilino-N-methyl-quinoline-3-carboxamide (210, 72.69 mg, 150.93 μmol) and 2-[[2-(2,6-dioxo-3-piperidyl)-1,3-dioxo-isoindolin-4-yl]amino]acetic acid (154, 50 mg, 150.93 μmol) in DMF (5 mL) were added DIPEA (58.52 mg, 452.79 μmol, 78.87 uL) and HATU (86.08 mg, 226.39 μmol) under nitrogen atmosphere at room temperature. The resulting mixture was stirred at room temperature for 16 h. After completion of reaction 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 4-anilino-6-[4-[4-[[2-[[2-(2,6-dioxo-3-piperidyl)-1,3-dioxo-isoindolin-4-yl]amino]acetyl]amino]butylcarbamoyl]-3-methyl-phenyl]-N-methyl-quinoline-3-carboxamide (Compound 353, 50 mg, 62.90 μmol, 41.68% yield). ¹H NMR (400 MHz, DMSO-d₆) δ 11.37 (s, 1H), 11.11 (s, 1H), 8.85 (s, 1H), 8.57 (d, J=6.6 Hz, 2H), 8.31 (q, J=7.1, 6.2 Hz, 2H), 8.15 (t, J=5.6 Hz, 1H), 8.05 (d, J=8.8 Hz, 1H), 7.63-7.52 (m, 2H), 7.50-7.43 (m, 3H), 7.41 (d, J=8.0 Hz, 1H), 7.36 (t, J=7.2 Hz, 1H), 7.30 (d, J=7.8 Hz, 2H), 7.06 (d, J=7.0 Hz, 1H), 6.96 (t, J=5.5 Hz, 1H), 6.86 (d, J=8.5 Hz, 1H), 5.07 (dd, J=12.9, 5.4 Hz, 1H), 3.93 (d, J=5.3 Hz, 2H), 3.28-3.19 (m, 2H), 3.19-3.09 (m, 2H), 2.89 (ddd, J=18.1, 14.1, 5.3 Hz, 1H), 2.68-2.56 (m, 2H), 2.42 (d, J=4.4 Hz, 3H), 2.39 (s, 3H), 2.08-1.96 (m, 1H), 1.56-1.43 (m, 4H). LCMS (ES+): m/z 795 [M+H]⁺

### Synthesis of Compound 354

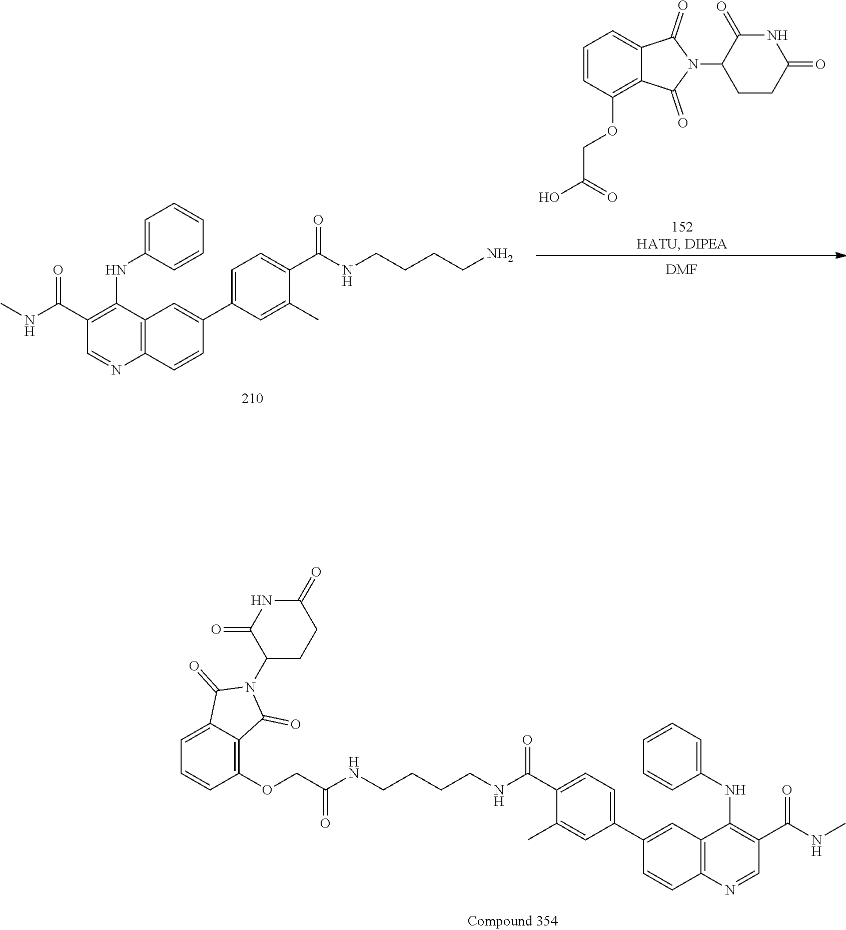

Into a 25 mL single-necked round-bottomed flask containing a well-stirred solution of 6-[4-(4-aminobutylcarbamoyl)-3-methyl-phenyl]-4-anilino-N-methyl-quinoline-3-carboxamide (210, 57.98 mg, 120.39 μmol) and 2-[2-(2,6-dioxo-3-piperidyl)-1,3-dioxo-isoindolin-4-yl]oxyacetic acid (152, 40 mg, 120.39 μmol) in DMF (5 mL) were added DIPEA (46.68 mg, 361.16 μmol, 62.91 uL) and HATU (68.66 mg, 180.58 μmol) under nitrogen atmosphere at room temperature. The resulting mixture was stirred at room temperature for 16 h. After completion of reaction, 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 4-anilino-6-[4-[4-[[2-[2-(2,6-dioxo-3-piperidyl)-1,3-dioxo-isoindolin-4-yl]oxyacetyl]amino]butylcarbamoyl]-3-methyl-phenyl]-N-methyl-quinoline-3-carboxamide (Compound 354, 30 mg, 37.70 μmol, 31.31% yield). ¹H NMR (400 MHz, DMSO-d₆) δ 11.42 (s, 1H), 11.12 (s, 1H), 8.85 (s, 1H), 8.61 (s, 1H), 8.58-8.53 (m, 1H), 8.38-8.28 (m, 2H), 8.06 (d, J=8.7 Hz, 1H), 8.01 (t, J=5.7 Hz, 1H), 7.81 (dd, J=8.5, 7.2 Hz, 1H), 7.56 (d, J=8.0 Hz, 1H), 7.51-7.44 (m, 4H), 7.44-7.35 (m, 2H), 7.31 (d, J=7.8 Hz, 2H), 5.12 (dd, J=13.0, 5.4 Hz, 1H), 4.78 (s, 2H), 3.28-3.15 (m, 4H), 2.94-2.81 (m, 1H), 2.69-2.54 (m, 2H), 2.44-2.35 (m, 6H), 2.06-1.97 (m, 1H), 1.58-1.47 (m, 4H). LCMS (ES+): m/z 796 [M+H]⁺

### Synthesis of Compound 355

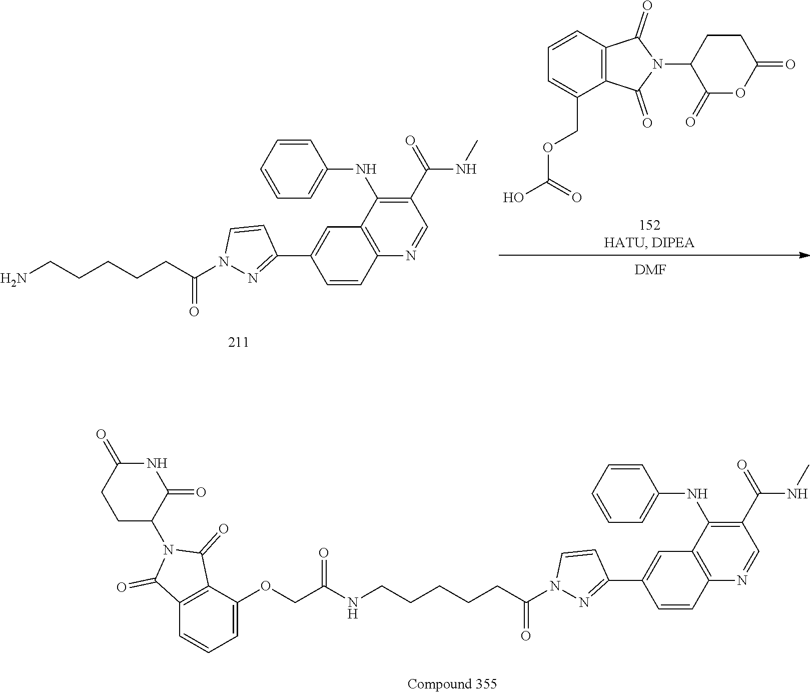

To a stirred solution of [2-(2,6-dioxotetrahydropyran-3-yl)-1,3-dioxo-isoindolin-4-yl]methyl hydrogen carbonate (152, 40 mg, 120.03 μmol) and 6-[1-(6-aminohexanoyl)pyrazol-3-yl]-4-anilino-N-methyl-quinoline-3-carboxamide (211, 54.80 mg, 120.03 μmol) in DMF (5.0 mL) was added DIPEA (77.57 mg, 600.15 μmol, 104.54 uL) and HATU (68.46 mg, 180.05 μmol). The resulting mixture was stirred for 16 hr at 25° C. The resulting solution was diluted with water (25 ml) and extracted with ethyl acetate (3×15 mL). The combined organic extracts were washed with water and brine solution, dried over anhydrous sodium sulfate, filtered and evaporated the solvent completely under reduced pressure. The resulting crude was purified bt reverse phase prep HPLC to yield 4-anilino-6-[1-[6-[[2-[2-(2,6-dioxo-3-piperidyl)-1,3-dioxo-isoindolin-4-yl]oxyacetyl]amino]hexanoyl]pyrazol-3-yl]-N-methyl-quinoline-3-carboxamide (Compound 355, 10 mg, 11.16 μmol, 9.30% yield) as an yellow solid. ¹H NMR (400 MHz, Methanol-d₄) δ 8.74 (s, 1H), 8.70 (s, 1H), 8.54 (dd, J=8.8, 1.8 Hz, 1H), 8.37 (d, J=2.9 Hz, 1H), 8.12-8.06 (m, 1H), 7.97 (d, J=8.8 Hz, 1H), 7.82-7.74 (m, 1H), 7.55-7.47 (m, 4H), 7.47-7.42 (m, 1H), 7.41-7.34 (m, 3H), 6.82 (s, 2H), 5.08 (dd, J=11.9, 5.5 Hz, 1H), 4.72 (s, 2H), 3.42-3.34 (m, 2H), 3.17 (t, J=7.2 Hz, 3H), 2.83-2.69 (m, 3H), 2.65 (s, 3H), 2.15-2.05 (m, 1H), 1.84 (q, J=7.4 Hz, 2H), 1.74-1.61 (m, 2H), 1.61-1.47 (m, 2H). LCMS (ES+): m/z 771 [M+H]⁺

### Synthesis of Compound 356

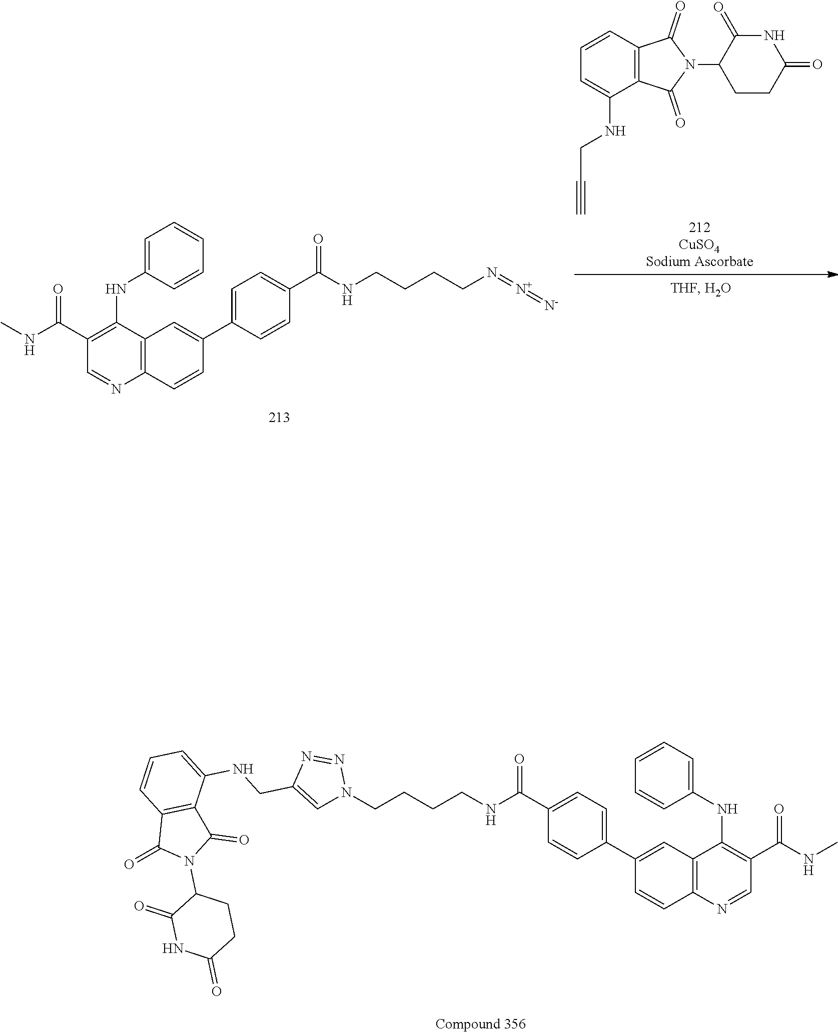

An oven dried round bottom flask was charged with a solution of 2-(2,6-dioxo-3-piperidyl)-4-(prop-2-ynylamino)isoindoline-1,3-dione (212, 41.00 mg, 131.70 μmol) in Water (1 mL) and THF (5 mL), 4-anilino-6-[4-(4-azidobutylcarbamoyl)phenyl]-N-methyl-quinoline-3-carboxamide (213, 65 mg, 131.70 μmol), Copper(II) sulfate (42.04 mg, 263.39 μmol, 11.68 uL) and Sodium ascorbate (52.18 mg, 263.39 μmol) were added at room temperature. The reaction mixture was stirred for 16 h at room temperature and the reaction mixture was filtered through celite and the filtrate was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[4-[4-[4-[[[2-(2,6-dioxo-3-piperidyl)-1,3-dioxo-isoindolin-4-yl]amino]methyl]triazol-1-yl]butylcarbamoyl]phenyl]-N-methyl-quinoline-3-carboxamide (Compound 356, 28 mg, 33.42 μmol, 25.38% yield) as yellow solid. ¹H NMR (400 MHz, DMSO-d₆) δ 11.27 (s, 1H), 11.10 (s, 1H), 8.83 (s, 1H), 8.75 (s, 1H), 8.59 (t, J=5.6 Hz, 1H), 8.54-8.48 (m, 1H), 8.39 (d, J=8.9 Hz, 1H), 8.08 (d, J=8.8 Hz, 1H), 8.04 (s, 1H), 8.00-7.95 (m, 2H), 7.81 (d, J=8.1 Hz, 2H), 7.60-7.51 (m, 1H), 7.45 (t, J=7.6 Hz, 2H), 7.36-7.31 (m, 1H), 7.29 (d, J=7.8 Hz, 2H), 7.15 (d, J=8.6 Hz, 1H), 7.08 (t, J=6.2 Hz, 1H), 7.03 (d, J=7.1 Hz, 1H), 5.05 (dd, J=12.9, 5.3 Hz, 1H), 4.59 (d, J=5.8 Hz, 2H), 4.37 (t, J=7.0 Hz, 2H), 3.29 (q, J=6.6 Hz, 2H), 2.88 (ddd, J=18.4, 14.2, 5.4 Hz, 2H), 2.70-2.56 (m, 2H), 2.36 (d, J=4.5 Hz, 3H), 2.07-1.97 (m, 1H), 1.84 (p, J=7.1 Hz, 2H), 1.48 (p, J=7.1 Hz, 2H). LCMS (ES+): m/z 805 [M+H]⁺

### Synthesis of Compound 357

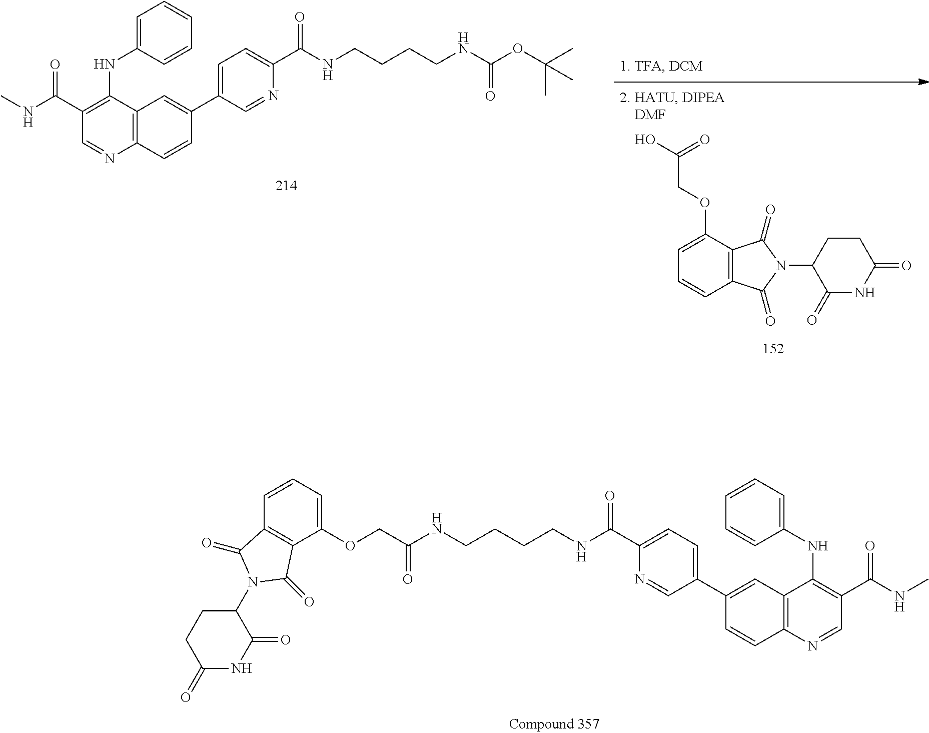 [SMILES: CNC(=O)c1cnc2ccc(-c3ccc(C(=O)NCCCCNC(=O)OC(C)(C)C)nc3)cc2c1Nc1ccccc1 ; O=C(O)COc1cccc2c1C(=O)N(C1CCC(=O)NC1=O)C2=O ; CNC(=O)c1cnc2ccc(-c3ccc(C(=O)NCCCCNC(=O)COc4cccc5c4C(=O)N(C4CCC(=O)NC4=O)C5=O)nc3)cc2c1Nc1ccccc1]

An oven dried round bottom flask was charged with a solution of tert-butyl N-[4-[[5-[4-anilino-3-(methylcarbamoyl)-6-quinolyl]pyridine-2-carbonyl]amino]butyl]carbamate (214, 171.15 mg, 300.96 μmol) in Dichloromethane (3 mL), Trifluoroacetic acid (4.44 g, 38.94 mmol, 3 mL) was added at room temperature. The reaction mixture was stirred for an hour at room temperature and the reaction mixture was concentrated under reduced pressure. To a solution of crude product in DMF (3 mL), 2-[2-(2,6-dioxo-3-piperidyl)-1,3-dioxo-isoindolin-4-yl]oxyacetic acid (152, 100 mg, 300.96 μmol), DIPEA (194.48 mg, 1.50 mmol, 262.11 uL) and HATU (137.32 mg, 361.16 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[6-[4-[[2-[2-(2,6-dioxo-3-piperidyl)-1,3-dioxo-isoindolin-4-yl]oxyacetyl]amino]butylcarbamoyl]-3-pyridyl]-N-methyl-quinoline-3-carboxamide (Compound 357, 70 mg, 85.76 μmol, 28.49% yield) as yellow solid. ¹H NMR (400 MHz), δ 11.10 (s, 1H), 10.07 (s, 1H), 8.87-8.80 (m, 2H), 8.75 (d, J=2.2 Hz, 1H), 8.54 (s, 1H), 8.36 (s, 1H), 8.21-8.13 (m, 2H), 8.10-8.04 (m, 2H), 8.00 (t, J=5.8 Hz, 1H), 7.81 (t, J=7.8 Hz, 1H), 7.47 (d, J=7.3 Hz, 1H), 7.38 (d, J=8.6 Hz, 1H), 7.31 (t, J=7.6 Hz, 2H), 7.12-7.03 (m, 3H), 5.11 (dd, J=12.8, 5.3 Hz, 1H), 4.77 (s, 2H), 3.18 (dd, J=12.3, 6.0 Hz, 2H), 2.96-2.82 (m, 2H), 2.70-2.60 (m, 2H), 2.56 (d, J=2.9 Hz, 3H), 2.09-1.97 (m, 1H), 1.51 (dd, J=28.9, 7.1 Hz, 5H). LCMS (ES+): m/z 783 [M+H]⁺

### Synthesis of Compound 358

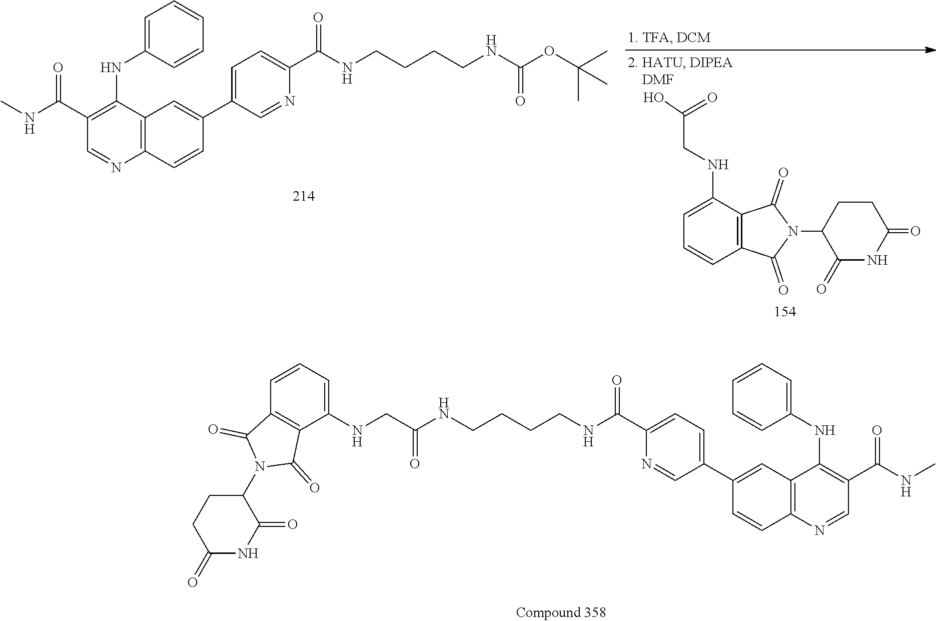 [SMILES: CNC(=O)c1cnc2ccc(-c3ccc(C(=O)NCCCCNC(=O)OC(C)(C)C)nc3)cc2c1Nc1ccccc1 ; O=C(O)CNc1cccc2c1C(=O)N(C1CCC(=O)NC1=O)C2=O ; CNC(=O)c1cnc2ccc(-c3ccc(C(=O)NCCCCNC(=O)CNc4cccc5c4C(=O)N(C4CCC(=O)NC4=O)C5=O)nc3)cc2c1Nc1ccccc1]

An oven dried round bottom flask was charged with a solution of tert-butyl N-[4-[[5-[4-anilino-3-(methylcarbamoyl)-6-quinolyl]pyridine-2-carbonyl]amino]butyl]carbamate (214, 171.66 mg, 301.86 μmol) in Dichloromethane (3 mL), Trifluoroacetic acid (4.44 g, 38.94 mmol, 3 mL) was added at room temperature. The reaction mixture was stirred for an hour at room temperature and the reaction mixture was concentrated under reduced pressure. To a solution of crude product in DMF (3 mL), 2-[[2-(2,6-dioxo-3-piperidyl)-1,3-dioxo-isoindolin-4-yl]amino]acetic acid (154, 100 mg, 301.86 μmol), DIPEA (195.06 mg, 1.51 mmol, 262.89 uL) and HATU (137.73 mg, 362.23 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[6-[4-[[2-[[2-(2,6-dioxo-3-piperidyl)-1,3-dioxo-isoindolin-4-yl]amino]acetyl]amino]butylcarbamoyl]-3-pyridyl]-N-methyl-quinoline-3-carboxamide (Compound 358, 22 mg, 26.86 μmol, 8.90% yield) as yellow solid. ¹H NMR (400 MHz, DMSO-d₆) δ 11.19 (s, 1H), 11.10 (s, 1H), 8.99 (d, J=2.3 Hz, 1H), 8.91-8.82 (m, 3H), 8.50-8.44 (m, 2H), 8.38-8.32 (m, 1H), 8.17 (d, J=8.2 Hz, 1H), 8.15-8.10 (m, 2H), 7.58 (t, J=7.8 Hz, 1H), 7.44 (t, J=7.6 Hz, 2H), 7.33 (t, J=7.7 Hz, 1H), 7.28 (d, J=7.8 Hz, 2H), 7.04 (d, J=7.1 Hz, 1H), 6.97-6.92 (m, 1H), 6.85 (d, J=8.6 Hz, 1H), 5.06 (dd, J=12.9, 5.4 Hz, 1H), 3.92 (d, J=5.0 Hz, 2H), 3.33 (q, J=6.6 Hz, 2H), 3.13 (q, J=6.5 Hz, 2H), 2.88 (ddd, J=17.8, 14.1, 5.5 Hz, 2H), 2.69-2.56 (m, 2H), 2.33 (d, J=4.4 Hz, 3H), 2.06-1.97 (m, 1H), 1.59-1.50 (m, 2H), 1.45 (t, J=7.6 Hz, 2H). LCMS (ES+): m/z 782 [M+H]⁺

### Synthesis of Compound 359

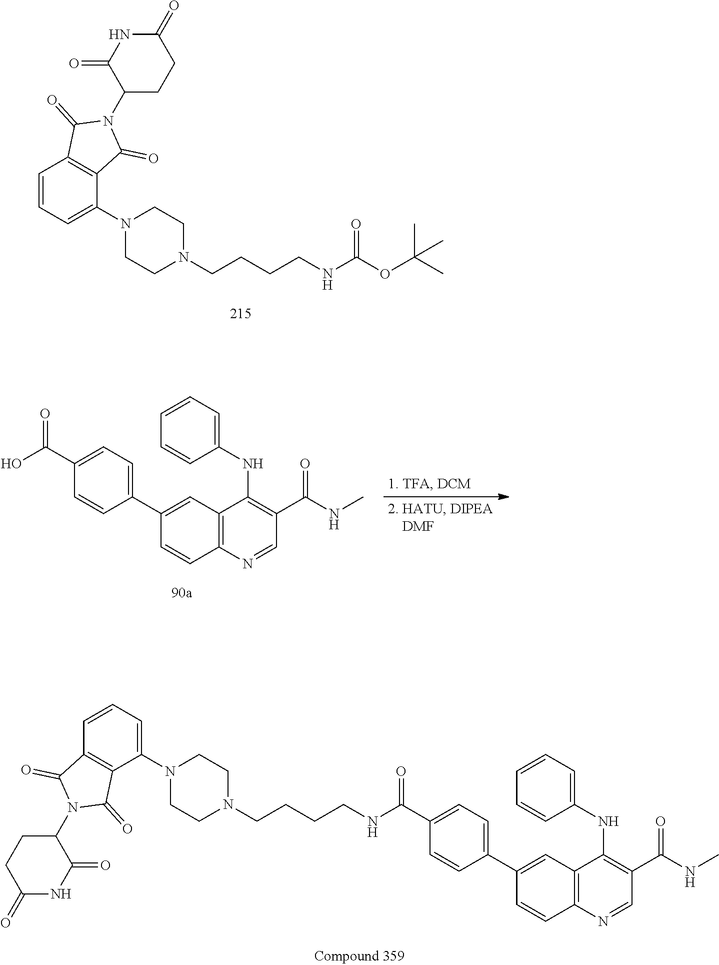 [SMILES: CC(C)(C)OC(=O)NCCCCN1CCN(c2cccc3c2C(=O)N(C2CCC(=O)NC2=O)C3=O)CC1 ; CNC(=O)c1cnc2ccc(-c3ccc(C(=O)O)cc3)cc2c1Nc1ccccc1 ; CNC(=O)c1cnc2ccc(-c3ccc(C(=O)NCCCCN4CCN(c5cccc6c5C(=O)N(C5CCC(=O)NC5=O)C6=O)CC4)cc3)cc2c1Nc1ccccc1]

An oven dried round bottom flask was charged with a solution of tert-butyl N-[4-[4-[2-(2,6-dioxo-3-piperidyl)-1,3-dioxo-isoindolin-4-yl]piperazin-1-yl]butyl]carbamate (215, 51.69 mg, 100.65 μmol) in Dichloromethane (3 mL), Trifluoroacetic acid (11.48 mg, 100.65 μmol, 7.75 uL) was added at room temperature. The reaction mixture was stirred for an hour at room temperature and the reaction mixture was concentrated under reduced pressure. To a solution of crude product in DMF (2 mL), 4-[4-anilino-3-(methylcarbamoyl)-6-quinolyl]benzoic acid (90a, 40 mg, 100.65 μmol), DIPEA (65.04 mg, 503.25 μmol, 87.65 uL) and HATU (45.92 mg, 120.78 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[4-[4-[4-[2-(2,6-dioxo-3-piperidyl)-1,3-dioxo-isoindolin-4-yl]piperazin-1-yl]butylcarbamoyl]phenyl]-N-methyl-quinoline-3-carboxamide (Compound 359, 4.77 mg, 5.91 μmol, 5.87% yield) as yellow solid. ¹H NMR (400 MHz, DMSO-d₆) δ 10.79 (s, 1H), 10.19 (s, 1H), 8.90 (s, 1H), 8.39 (s, 1H), 8.23 (d, J=4.8 Hz, 2H), 8.14 (d, J=1.8 Hz, 1H), 8.07-8.00 (m, 2H), 7.87 (d, J=8.1 Hz, 2H), 7.71-7.65 (m, 1H), 7.52 (d, J=8.0 Hz, 2H), 7.35-7.28 (m, 5H), 7.11 (t, J=7.5 Hz, 1H), 7.07 (d, J=7.9 Hz, 2H), 5.05 (dd, J=12.6, 5.5 Hz, 1H), 3.78 (s, 1H), 3.40-3.30 (m, 7H), 3.20 (s, 3H), 2.87 (ddd, J=17.3, 13.7, 5.5 Hz, 2H), 2.72 (d, J=4.6 Hz, 4H), 2.68-2.56 (m, 7H), 2.10-2.01 (m, 1H), 1.92 (s, 1H), 1.67-1.53 (m, 5H). LCMS (ES+): m/z 793 [M+H]⁺

### Synthesis of Compound 360

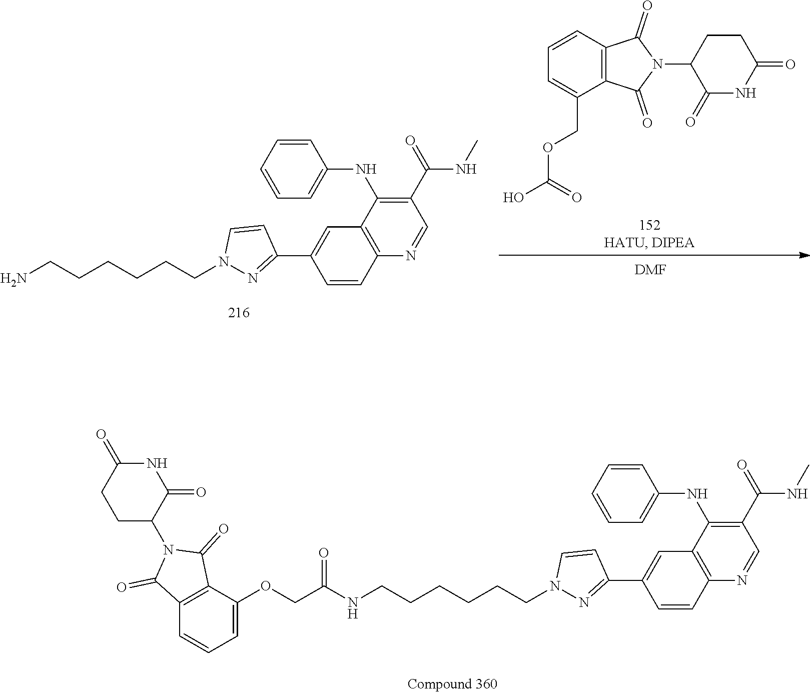

To a stirred solution of 2-[2-(2,6-dioxo-3-piperidyl)-1,3-dioxo-isoindolin-4-yl]oxyacetic acid (152, 100 mg, 300.96 μmol) and 6-[1-(6-aminohexyl)pyrazol-4-yl]-4-anilino-N-methyl-quinoline-3-carboxamide (216, 133.19 mg, 300.96 μmol) in DMF (10.0 mL) was added DIPEA (194.49 mg, 1.50 mmol, 262.11 uL) and HATU (171.65 mg, 451.45 μmop. The resulting mixture was stirred for 16 hr at 25° C. The resulting mixture was diluted with water (20 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase prep HPLC to yield 4-anilino-6-[1-[6-[[2-[2-(2,6-dioxo-3-piperidyl)-1,3-dioxo-isoindolin-4-yl]oxyacetyl]amino]hexyl]pyrazol-4-yl]-N-methyl-quinoline-3-carboxamide (Compound 360, 20 mg, 26.10 μmol, 8.67% yield) as an yellow solid. ¹H NMR (400 MHz, Methanol-d₄) δ 8.64 (s, 1H), 8.14 (d, J=1.9 Hz, 1H), 7.92 (dd, J=8.8, 1.9 Hz, 1H), 7.89 (s, 1H), 7.85 (d, J=8.7 Hz, 1H), 7.73 (dd, J=8.5, 7.3 Hz, 1H), 7.65 (s, 1H), 7.46 (d, J=7.3 Hz, 1H), 7.37-7.31 (m, 3H), 7.17-7.09 (m, 3H), 5.11 (dd, J=12.5, 5.4 Hz, 1H), 4.69 (s, 2H), 4.16 (t, J=6.8 Hz, 2H), 2.87-2.70 (m, 4H), 2.67 (d, J=5.2 Hz, 1H), 2.14-2.05 (m, 1H), 1.88 (p, J=6.9 Hz, 2H), 1.63-1.53 (m, 2H), 1.45-1.27 (m, 5H). LCMS (ES+): m/z 757 [M+H]⁺

### Synthesis of Compound 361

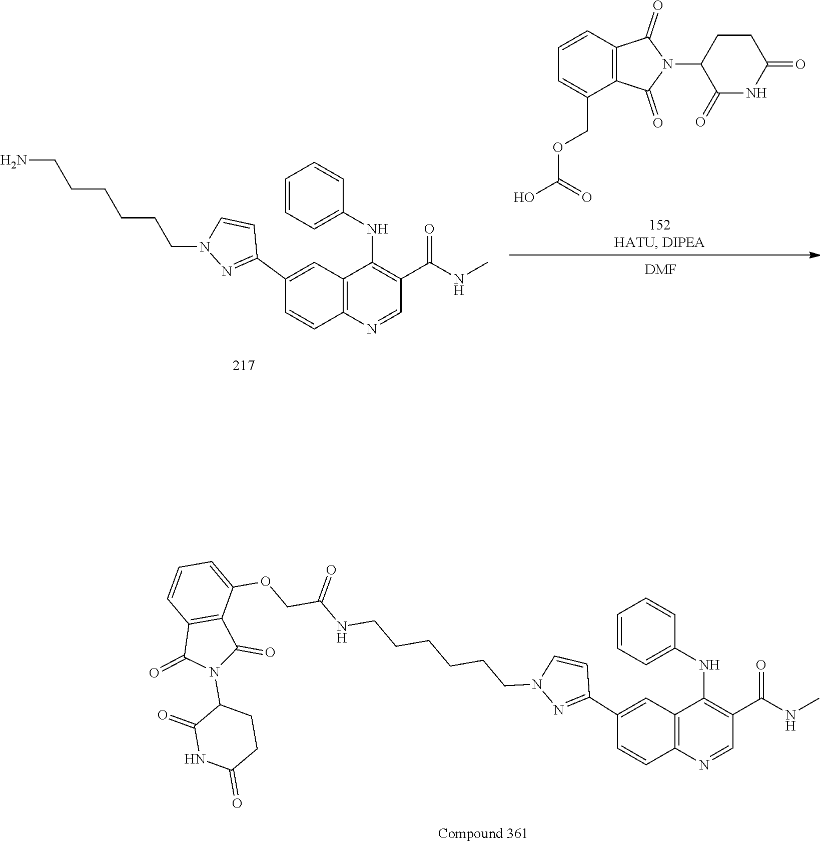

To a stirred solution of 6-[1-(6-aminohexyl)pyrazol-3-yl]-4-anilino-N-methyl-quinoline-3-carboxamide (217, 100 mg, 225.96 μmol) and 2-[2-(2,6-dioxo-3-piperidyl)-1,3-dioxo-isoindolin-4-yl]oxyacetic acid (152, 75.08 mg, 225.96 μmol) in DMF (10.0 mL) was added DIPEA (146.02 mg, 1.13 mmol, 196.79 uL) and HATU (128.88 mg, 338.94 μmol) and stirred the mixture for 16 hr at 25° C. The resulting mixture was diluted with water (20 mL) and extracted with ethyl acetate (3×20 mL). The combined organic extracts were washed with water and brine solution, dried over anhydrous sodium sulfate, filtered and evaporated the solvent completely under reduced pressure. The resulting crude was purified by reverse phase prep HPLC to yield 4-anilino-6-[1-[6-[[2-[2-(2,6-dioxo-3-piperidyl)-1,3-dioxo-isoindolin-4-yl]oxyacetyl]amino]hexyl]pyrazol-3-yl]-N-methyl-quinoline-3-carboxamide (Compound 361, 25 mg, 32.03 μmol, 14.18% yield) as an yellow solid. ¹H NMR (400 MHz, Methanol-d₄) δ 8.67 (s, 1H), 8.38 (d, J=1.9 Hz, 1H), 8.19 (dd, J=8.8, 1.9 Hz, 1H), 7.91 (d, J=8.8 Hz, 1H), 7.77 (dd, J=8.5, 7.3 Hz, 1H), 7.60 (d, J=2.4 Hz, 1H), 7.49 (d, J=7.2 Hz, 1H), 7.38 (d, J=8.4 Hz, 1H), 7.35-7.29 (m, 2H), 7.14-7.09 (m, 3H), 6.39 (d, J=2.4 Hz, 1H), 5.10 (dd, J=12.3, 5.5 Hz, 1H), 4.71 (s, 3H), 4.16 (t, J=6.9 Hz, 3H), 2.85-2.71 (m, 4H), 2.66 (d, J=2.0 Hz, 4H), 2.10 (d, J=4.9 Hz, 1H), 1.95 (s, 1H), 1.93-1.83 (m, 2H), 1.62-1.52 (m, 2H), 1.48-1.27 (m, 5H). LCMS (ES+): m/z 757 [M+H]⁺

### Synthesis of Compound 362

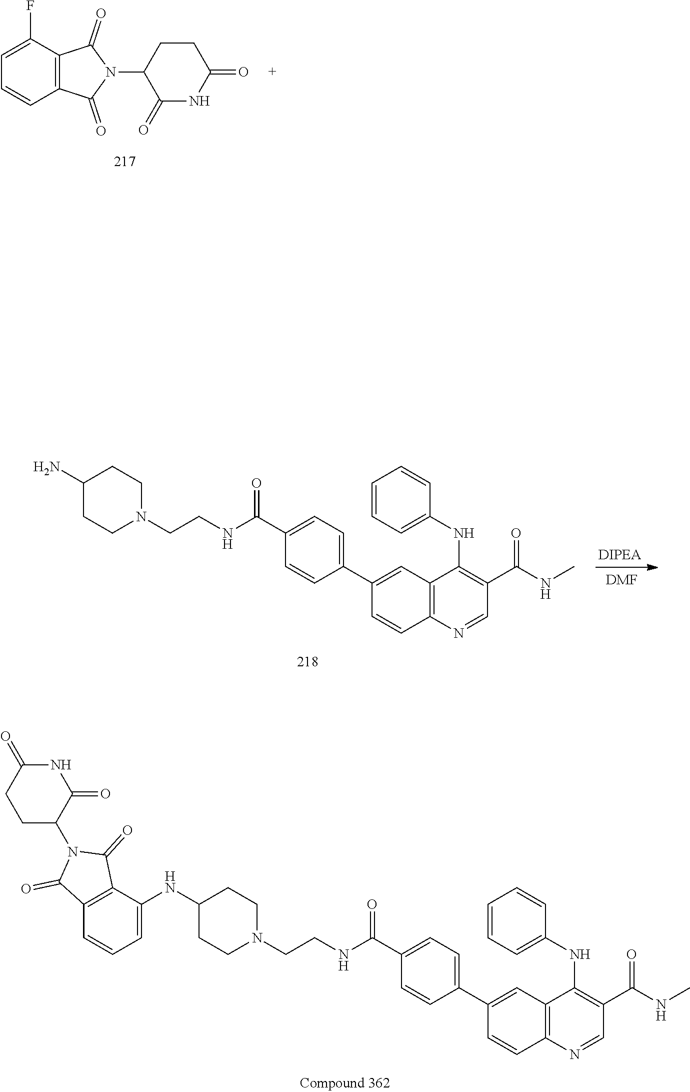

Into a 50 mL single-necked round-bottomed flask containing a well-stirred solution of 6-[4-[2-(4-amino-1-piperidyl)ethylcarbamoyl]phenyl]-4-anilino-N-methyl-quinoline-3-carboxamide (218, 150 mg, 287.00 μmol) and 2-(2,6-dioxo-3-piperidyl)-4-fluoro-isoindoline-1,3-dione (217, 95.13 mg, 344.41 μmol) in anhydrous DMF (5 mL) was added DIPEA (185.47 mg, 1.44 mmol, 249.96 uL) under nitrogen atmosphere. The resulting mixture was heated at 100° C. for 16 h. The reaction mixture was concentrated under reduced pressure and purified by prep-HPLC (ATLANTIS OBD C18(19×150)MM 5μ) Mobile phase: A: 0.1% HCOOH in water B: ACN to afford 4-anilino-6-[4-[2-[4-[[2-(2,6-dioxo-3-piperidyl)-1,3-dioxo-isoindolin-4-yl]amino]-1-piperidyl]ethylcarbamoyl]phenyl]-N-methyl-quinoline-3-carboxamide (Compound 362, 5 mg, 6.42 μmol, 2.24% yield). ¹H NMR (400 MHz, DMSO-d₆) δ 11.11 (s, 1H), 9.33 (s, 1H), 8.90-8.82 (m, 2H), 8.63 (s, 1H), 8.55 (d, J=5.0 Hz, 1H), 8.33 (d, J=8.7 Hz, 1H), 8.08 (d, J=8.9 Hz, 1H), 7.99 (d, J=8.2 Hz, 2H), 7.81 (d, J=8.0 Hz, 2H), 7.76 (s, 1H), 7.67-7.61 (m, 1H), 7.43 (d, J=7.7 Hz, 2H), 7.39-7.33 (m, 2H), 7.33-7.28 (m, 1H), 7.24 (d, J=8.4 Hz, 3H), 7.11 (d, J=7.0 Hz, 1H), 6.30 (d, J=8.0 Hz, 1H), 5.06 (dd, J=12.7, 5.4 Hz, 1H), 3.69 (d, J=18.9 Hz, 4H), 3.08-2.95 (m, 2H), 2.95-2.83 (m, 1H), 2.68-2.56 (m, 2H), 2.44 (d, J=4.7 Hz, 3H), 2.33 (p, J=1.8 Hz, 1H), 2.27-2.18 (m, 2H), 2.07-1.97 (m, 2H), 1.92-1.69 (m, 3H), 1.38-1.28 (m, 1H), 1.16-1.09 (m, 1H). LCMS (ES+): m/z 779 [M+H]⁺

### Synthesis of Compound 363

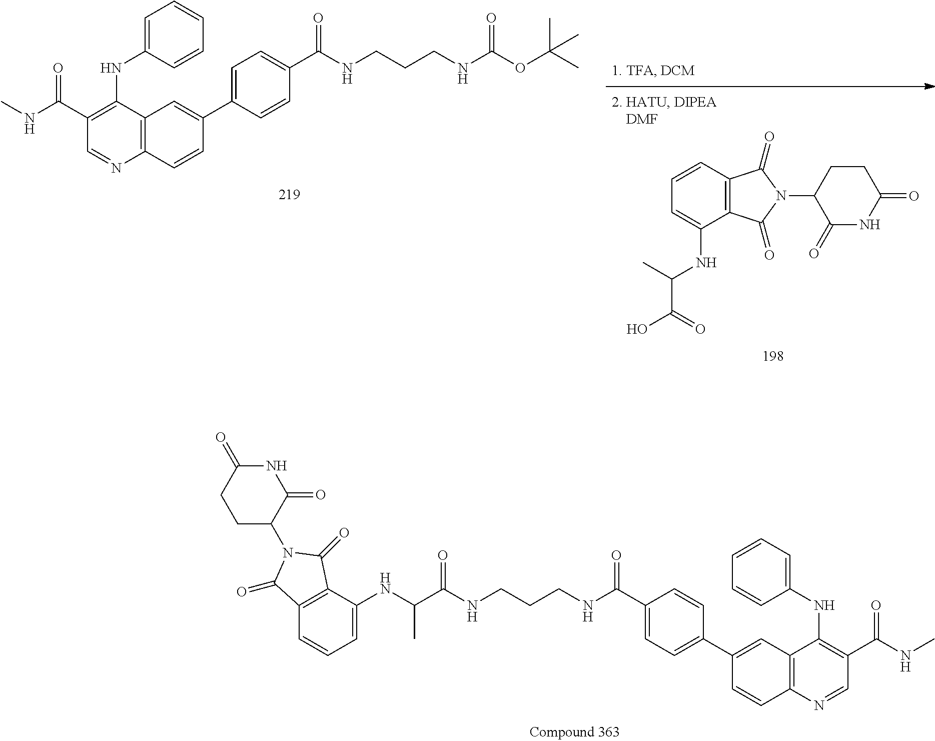 [SMILES: CNC(=O)c1cnc2ccc(-c3ccc(C(=O)NCCCNC(=O)OC(C)(C)C)cc3)cc2c1Nc1ccccc1 ; CC(Nc1cccc2c1C(=O)N(C1CCC(=O)NC1=O)C2=O)C(=O)O ; CNC(=O)c1cnc2ccc(-c3ccc(C(=O)NCCCNC(=O)C(C)Nc4cccc5c4C(=O)N(C4CCC(=O)NC4=O)C5=O)cc3)cc2c1Nc1ccccc1]

An oven dried round bottom flask was charged with a solution of tert-butyl N-[3-[[4-[4-anilino-3-(methylcarbamoyl)-6-quinolyl]benzoyl]amino]propyl]carbamate (219, 115.44 mg, 208.51 μmol) in Dichloromethane (3 mL), Trifluoroacetic acid (2.96 g, 25.96 mmol, 2 mL) was added at room temperature. The reaction mixture was stirred for an hour at room temperature and the reaction mixture was concentrated under reduced pressure. To a solution of crude product in DMF (2 mL), 2-[[2-(2,6-dioxo-3-piperidyl)-1,3-dioxo-isoindolin-4-yl]amino]propanoic acid (198, 60 mg, 173.76 μmol), DIPEA (112.28 mg, 868.79 μmol, 151.32 uL) and HATU (99.10 mg, 260.64 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[4-[3-[2-[[2-(2,6-dioxo-3-piperidyl)-1,3-dioxo-isoindolin-4-yl]amino]propanoylamino]propylcarbamoyl]phenyl]-N-methyl-quinoline-3-carboxamide as (Compound 363, 29.32 mg, 35.56 μmol, 20.47% yield) yellow solid. ¹H NMR (400 MHz, DMSO-d₆) δ 11.11 (s, 1H), 8.82 (s, 1H), 8.52 (d, J=6.0 Hz, 2H), 8.42 (s, 1H), 8.31-8.24 (m, 1H), 8.23-8.16 (m, 1H), 8.03 (d, J=8.8 Hz, 1H), 7.91 (d, J=8.0 Hz, 2H), 7.72-7.66 (m, 2H), 7.64-7.59 (m, 1H), 7.36 (t, J=7.7 Hz, 2H), 7.21-7.11 (m, 2H), 7.09 (d, J=7.1 Hz, 1H), 6.94 (d, J=8.5 Hz, 1H), 6.74 (t, J=6.8 Hz, 1H), 5.07 (dd, J=12.9, 5.4 Hz, 1H), 4.25-4.16 (m, 1H), 3.29-3.24 (m, 2H), 3.21-3.15 (m, 2H), 3.04-2.81 (m, 3H), 2.68-2.57 (m, 2H), 2.35-2.29 (m, 1H), 2.08-1.96 (m, 1H), 1.74-1.63 (m, 2H), 1.45-1.36 (m, 3H), 0.84 (d, J=7.8 Hz, 1H). LCMS (ES+): m/z 780 [M+H]⁺

### Synthesis of Compound 364

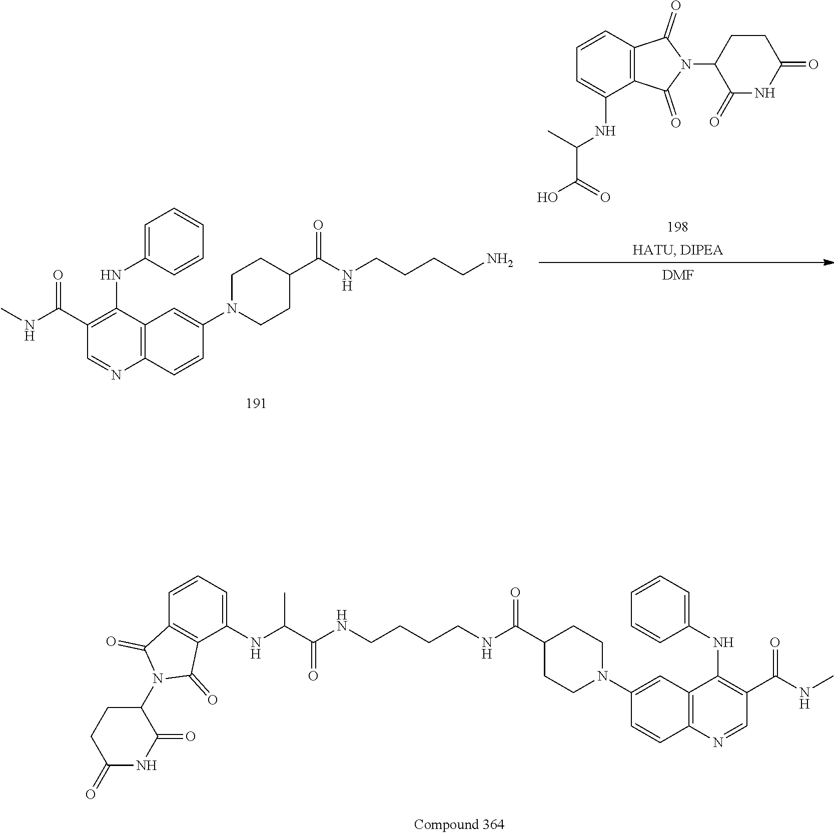

To a stirred solution of 2-[[2-(2,6-dioxo-3-piperidyl)-1,3-dioxo-isoindolin-4-yl]amino]propanoic acid (198, 115 mg, 333.04 μmol) and 6-[4-(4-aminobutylcarbamoyl)-1-piperidyl]-4-anilino-N-methyl-quinoline-3-carboxamide (191, 173.86 mg, 366.34 μmol) in DMF (5 mL) was added DIPEA (215.21 mg, 1.67 mmol, 290.05 uL) and HATU (189.95 mg, 499.56 μmol). The resulting mixture was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[4-[4-[2-[[2-(2,6-dioxo-3-piperidyl)-1,3-dioxo-isoindolin-4-yl]amino]propanoylamino]butylcarbamoyl]-1-piperidyl]-N-methyl-quinoline-3-carboxamide (Compound 364, 60 mg, 69.18 μmol, 20.77% yield) as an yellow solid. LCMS (ES+): m/z 802 [M+H]⁺

### Synthesis of Compound 365

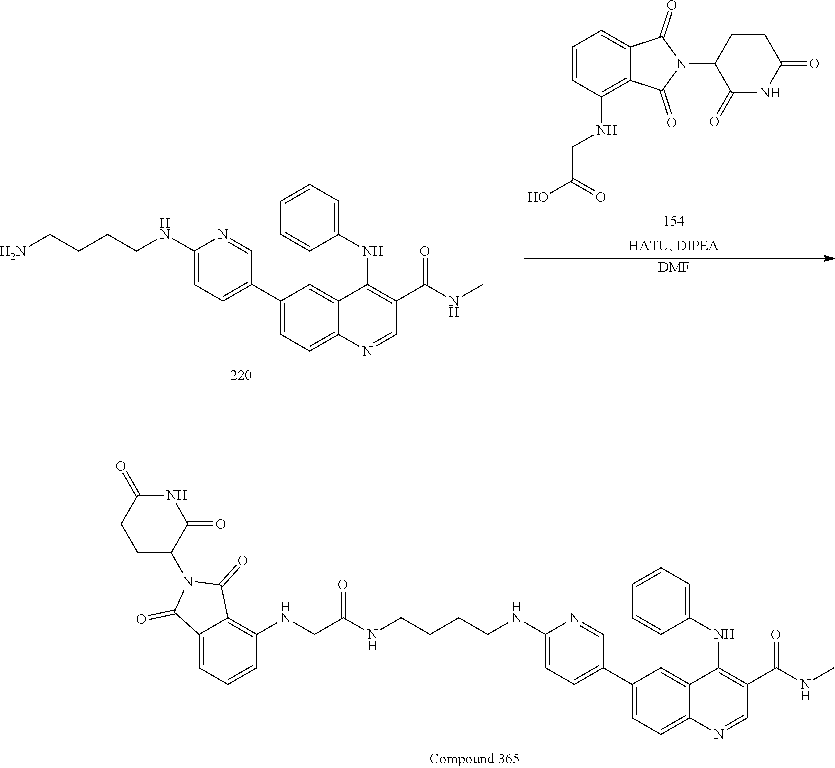

To a stirred solution of 6-[6-(4-aminobutylamino)-3-pyridyl]-4-anilino-N-methyl-quinoline-3-carboxamide (220, 100 mg, 226.99 μmol), 2-[[2-(2,6-dioxo-3-piperidyl)-1,3-dioxo-isoindolin-4-yl]amino]acetic acid (154, 112.80 mg, 340.49 μmol) in DMF (10 mL) was added DIPEA (146.68 mg, 1.13 mmol, 197.69 uL) and HATU (129.47 mg, 340.49 μmol). The reaction was stirred for 16 hr at 25° C. The reaction mixture was diluted with water (25 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[6-[4-[[2-[[2-(2,6-dioxo-3-piperidyl)-1,3-dioxo-isoindolin-4-yl]amino]acetyl]amino]butylamino]-3-pyridyl]-N-methyl-quinoline-3-carboxamide (Compound 365, 19.46 mg, 23.36 μmol, 10.29% yield) as a yellow solid. ¹H NMR (400 MHz, DMSO-d₆) δ 11.11 (s, 1H), 8.78 (s, 1H), 8.68 (s, 1H), 8.48-8.41 (m, 1H), 8.36 (s, 1H), 8.31 (d, J=8.8 Hz, 1H), 8.15 (t, J=6.2 Hz, 1H), 8.03 (d, J=8.9 Hz, 1H), 7.60-7.54 (m, 1H), 7.44 (t, J=7.6 Hz, 2H), 7.32 (t, J=7.7 Hz, 1H), 7.27 (d, J=7.7 Hz, 2H), 7.05 (d, J=7.1 Hz, 1H), 6.98-6.92 (m, 1H), 6.85 (d, J=8.5 Hz, 1H), 5.06 (dd, J=12.8, 5.4 Hz, 1H), 3.92 (d, J=5.0 Hz, 2H), 3.37-3.29 (m, 2H), 3.20-3.11 (m, 2H), 2.95-2.83 (m, 1H), 2.69-2.57 (m, 2H), 2.35-2.29 (m, 4H), 2.06-1.95 (m, 1H), 1.63-1.46 (m, 4H). LCMS (ES+): m/z 754 [M+H]⁺

### Synthesis of Compound 366

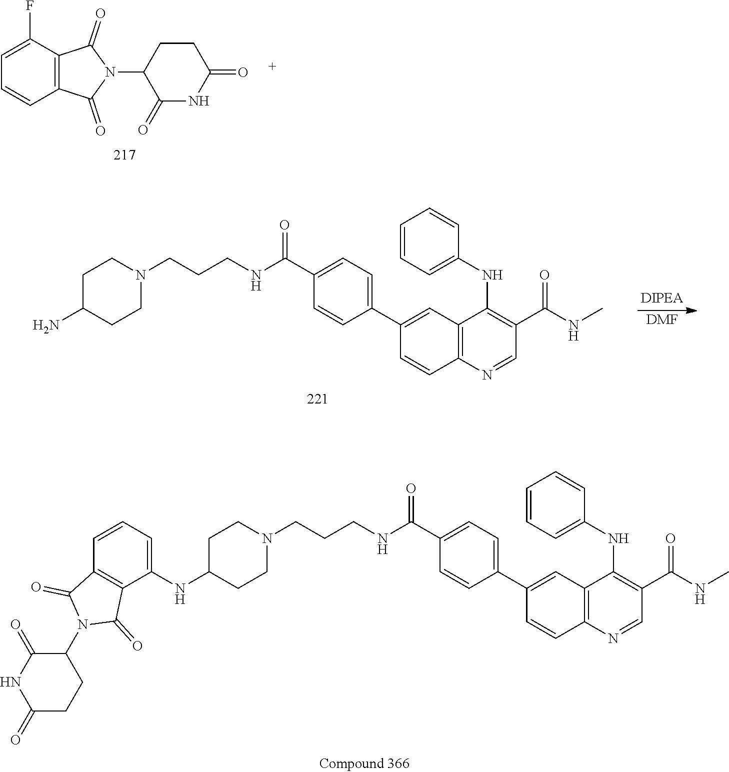

Into a 50 mL single-necked round-bottomed flask containing a well-stirred solution of 6-[4-[3-(4-amino-1-piperidyl)propylcarbamoyl]phenyl]-4-anilino-N-methyl-quinoline-3-carboxamide (221, 205.37 mg, 382.67 μmol) and 2-(2,6-dioxo-3-piperidyl)-4-fluoro-isoindoline-1,3-dione (217, 126.84 mg, 459.21 μmol) in anhydrous DMF (5 mL) was added DIPEA (247.29 mg, 1.91 mmol, 333.27 uL) under nitrogen atmosphere. The resulting mixture was heated at 100° C. for 16 h. The reaction mixture was concentrated under reduced pressure and purified by prep-HPLC (ATLANTIS OBD C18(19×150)MM 5μ) Mobile phase: A: 0.1% TFA in water B: ACN to afford 4-anilino-6-[4-[3-[4-[[2-(2,6-dioxo-3-piperidyl)-1,3-dioxo-isoindolin-4-yl]amino]-1-piperidyl]propylcarbamoyl]phenyl]-N-methyl-quinoline-3-carboxamide (Compound 366, 15 mg, 18.92 μmol, 4.94% yield) as a brown solid. ¹H NMR (400 MHz, DMSO-d₆) δ 11.11 (s, 1H), 8.85 (s, 1H), 8.74 (d, J=6.0 Hz, 1H), 8.62 (s, 1H), 8.57-8.52 (m, 1H), 8.37-8.28 (m, 1H), 8.07 (d, J=8.8 Hz, 1H), 7.97 (d, J=8.1 Hz, 2H), 7.78 (d, J=7.9 Hz, 2H), 7.64 (dd, J=8.5, 7.2 Hz, 1H), 7.42 (t, J=7.6 Hz, 2H), 7.34-7.21 (m, 4H), 7.17-7.08 (m, 1H), 6.30 (d, J=8.1 Hz, 1H), 5.06 (dd, J=12.7, 5.5 Hz, 1H), 3.89-3.79 (m, 1H), 3.60 (d, J=12.1 Hz, 2H), 3.21-3.02 (m, 3H), 2.96-2.83 (m, 2H), 2.67 (q, J=1.8 Hz, 1H), 2.63-2.56 (m, 2H), 2.45-2.39 (m, 2H), 2.33 (p, J=1.9 Hz, 1H), 2.20 (d, J=13.5 Hz, 2H), 2.09-1.89 (m, 4H), 1.72 (q, J=12.6 Hz, 2H). LCMS (ES+): m/z 793 [M+H]⁺

### Synthesis of Compound 367

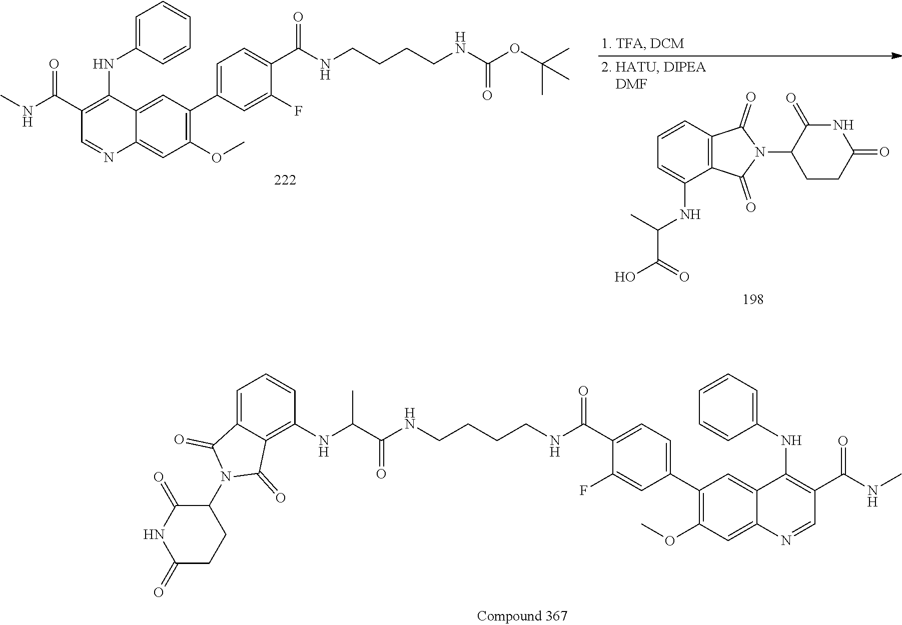 [SMILES: CNC(=O)c1cnc2cc(OC)c(-c3ccc(C(=O)NCCCCNC(=O)OC(C)(C)C)c(F)c3)cc2c1Nc1ccccc1 ; CC(Nc1cccc2c1C(=O)N(C1CCC(=O)NC1=O)C2=O)C(=O)O ; CNC(=O)c1cnc2cc(OC)c(-c3ccc(C(=O)NCCCCNC(=O)C(C)Nc4cccc5c4C(=O)N(C4CCC(=O)NC4=O)C5=O)c(F)c3)cc2c1Nc1ccccc1]

Into a 25 mL single-necked round-bottomed flask containing a well-stirred solution of tert-butyl N-[4-[[4-[4-anilino-7-methoxy-3-(methylcarbamoyl)-6-quinolyl]-2-fluoro-benzoyl]amino]butyl]carbamate (222, 50 mg, 81.21 μmol) in anhydrous DCM (3 mL) was added TFA (46.30 mg, 406.05 μmol, 31.28 uL) under nitrogen atmosphere at room temperature. The reaction mixture was stirred at room temperature for 30 minutes. The reaction mixture was concentrated under reduced pressure to afford a crude residue which was dissolved in DMF (2 mL). DIPEA (31.49 mg, 243.63 μmol, 42.44 uL), 2-[[2-(2,6-dioxo-3-piperidyl)-1,3-dioxo-isoindolin-4-yl]amino]propanoic acid (198, 30.85 mg, 89.33 μmol) and HATU (46.32 mg, 121.81 μmol) were added under nitrogen atmosphere at room temperature. The resulting mixture was stirred at room temperature for 16 h. Ice cold water (20 mL) was added and stirred for 10 minutes. The resulting solid was filtered and dried to afford 6-(4-((4-(2-((2-(2,6-dioxopiperidin-3-yl)-1,3-dioxoisoindolin-4-yl)amino)propanamido)butyl)carbamoyl)-3-fluorophenyl)-7-methoxy-N-methyl-4-(phenylamino)quinoline-3-carboxamide (Compound 367, 36 mg, 38.01 μmol, 46.81% yield) as a yellow solid. ¹H NMR (400 MHz, DMSO-d₆) δ 11.12 (s, 1H), 10.29 (s, 1H), 8.85 (s, 1H), 8.60 (q, J=4.6 Hz, 1H), 8.33 (t, J=6.6 Hz, 1H), 8.27-8.19 (m, 1H), 7.75 (s, 1H), 7.61 (t, J=7.9 Hz, 1H), 7.51 (t, J=7.8 Hz, 1H), 7.45 (s, 1H), 7.33-7.27 (m, 2H), 7.17-7.11 (m, 2H), 7.08 (d, J=6.6 Hz, 1H), 7.01 (d, J=7.9 Hz, 2H), 6.93 (d, J=8.5 Hz, 1H), 6.74 (t, J=7.3 Hz, 1H), 5.07 (dd, J=12.9, 5.3 Hz, 1H), 4.24-4.15 (m, 1H), 3.94 (s, 3H), 3.26-3.19 (m, 2H), 3.15-3.09 (m, 2H), 3.02-2.82 (m, 2H), 2.66 (d, J=4.8 Hz, 3H), 2.60-2.55 (m, 2H), 2.07-1.95 (m, 1H), 1.47 (s, 3H), 1.38 (d, J=6.8 Hz, 3H), 1.27-1.19 (m, 1H), 0.94 (d, J=6.5 Hz, 4H). LCMS (ES+): m/z 844 [M+H]⁺

### Synthesis of Compound 368

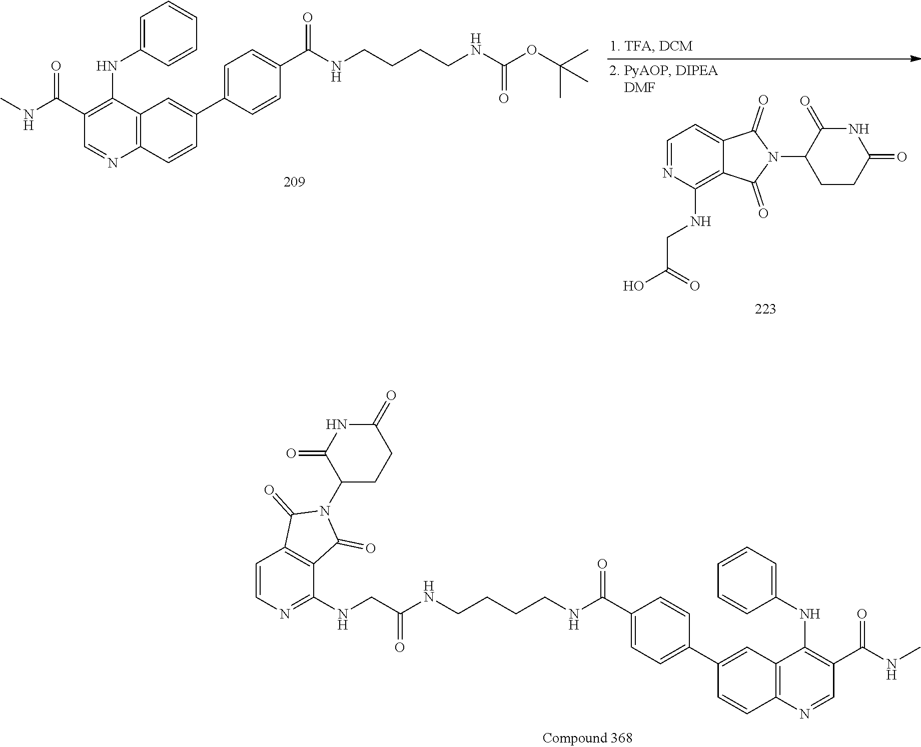 [SMILES: CNC(=O)c1cnc2ccc(-c3ccc(C(=O)NCCCCNC(=O)OC(C)(C)C)cc3)cc2c1Nc1ccccc1 ; O=C(O)CNc1nccc2c1C(=O)N(C1CCC(=O)NC1=O)C2=O ; CNC(=O)c1cnc2ccc(-c3ccc(C(=O)NCCCCNC(=O)CNc4nccc5c4C(=O)N(C4CCC(=O)NC4=O)C5=O)cc3)cc2c1Nc1ccccc1]

To a stirred solution of crude tert-butyl N-[4-[[4-[4-anilino-3-(methylcarbamoyl)-6-quinolyl]benzoyl]amino]butyl]carbamate (209, 100 mg, 176.16 μmol) in anhydrous Dichloromethane (5 mL) was added Trifluoroacetic acid (2.97 g, 26.05 mmol, 2.01 mL) at 0° C. The reaction mixture was stirred at room temperature for 3 hours. The reaction mixture was concentrated completely and this crude was taken as such for the next step. To the stirred solution of above crude in N,N-Dimethylformamide (6 mL) and Dichloromethane (5 mL) was added 2-[[2-(2,6-dioxo-3-piperidyl)-1,3-dioxo-pyrrolo[3,4-c]pyridin-4-yl]amino]acetic acid (223, 58.53 mg, 176.16 μmol) followed by PyAOP (137.77 mg, 264.24 μmol) and N,N-Diisopropylethylamine (113.83 mg, 880.78 μmol, 153.41 uL). The reaction mixture was stirred at room temperature for 16 hours. Ice cold water (15 mL) was added and stirred for 10 minutes, and then extracted with ethyl acetate. Then combined organics were dried over anhydrous sodium sulfate and then concentrated to yield the crude product which was purified by prep-HPLC(SUNFIRE OBD C18(100×30)MM 5μ) Mobile phase: A: 0.1% TFA in water B: ACN) to yield the product 4-anilino-6-[4-[4-[[2-[[2-(2,6-dioxo-3-piperidyl)-1,3-dioxo-pyrrolo[3,4-c]pyridin-4-yl]amino]acetyl]amino]butylcarbamoyl]phenyl]-N-methyl-quinoline-3-carboxamide (Compound 368, 5 mg, 6.20 μmol, 3.52% yield). LCMS (ES+): m/z 782 [M+H]⁺

### Synthesis of Compound 369

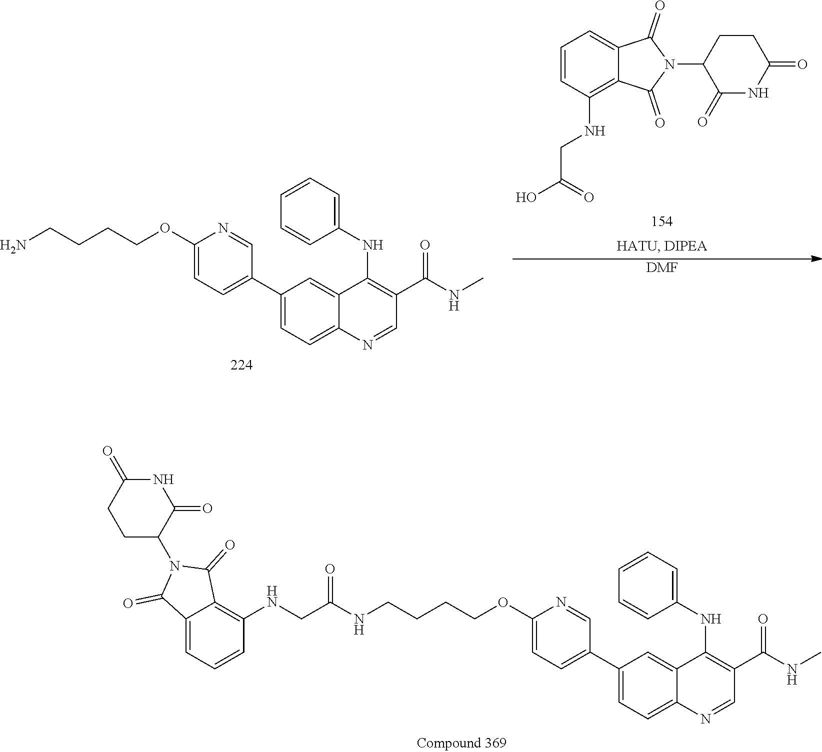

To a stirred solution of 6-[6-(4-aminobutoxy)-3-pyridyl]-4-anilino-N-methyl-quinoline-3-carboxamide (224, 100 mg, 226.49 μmol) and 2-[[2-(2,6-dioxo-3-piperidyl)-1,3-dioxo-isoindolin-4-yl]amino]acetic acid (154, 112.55 mg, 339.73 μmol) in DMF (10 mL) was added DIPEA (146.36 mg, 1.13 mmol, 197.25 uL) and HATU (129.18 mg, 339.73 μmol). The mixture was stirred for 16 hr at 25° C. The reaction mixture was diluted with water (25 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[6-[4-[[2-[[2-(2,6-dioxo-3-piperidyl)-1,3-dioxo-isoindolin-4-yl]amino]acetyl]amino]butoxy]-3-pyridyl]-N-methyl-quinoline-3-carboxamide (Compound 369, 21.09 mg, 27.35 μmol, 12.08% yield) as a yellow solid. ¹H NMR (400 MHz, DMSO-d₆) δ 11.18 (s, 1H), 11.11 (s, 1H), 8.79 (d, J=3.4 Hz, 2H), 8.59 (d, J=2.7 Hz, 1H), 8.48-8.42 (m, 1H), 8.38 (dd, J=8.8, 1.8 Hz, 1H), 8.16 (t, J=5.7 Hz, 1H), 8.12 (dd, J=8.7, 2.6 Hz, 1H), 8.06 (d, J=8.8 Hz, 1H), 7.57 (dd, J=8.5, 7.1 Hz, 1H), 7.45 (t, J=7.8 Hz, 2H), 7.36-7.31 (m, 1H), 7.31-7.25 (m, 2H), 7.05 (d, J=7.0 Hz, 1H), 6.98 (d, J=8.7 Hz, 1H), 6.95 (s, 1H), 6.86 (d, J=8.5 Hz, 1H), 5.07 (dd, J=12.9, 5.3 Hz, 1H), 4.32 (t, J=6.4 Hz, 2H), 3.93 (d, J=3.8 Hz, 3H), 3.17 (q, J=6.6 Hz, 2H), 2.89 (ddd, J=17.4, 13.9, 5.3 Hz, 1H), 2.68-2.56 (m, 2H), 2.29 (d, J=4.5 Hz, 3H), 2.06-1.98 (m, 1H), 1.73 (dd, J=8.8, 6.0 Hz, 2H), 1.57 (dd, J=8.9, 6.1 Hz, 2H). LCMS (ES+): m/z 755 [M+H]⁺

### Synthesis of Compound 372

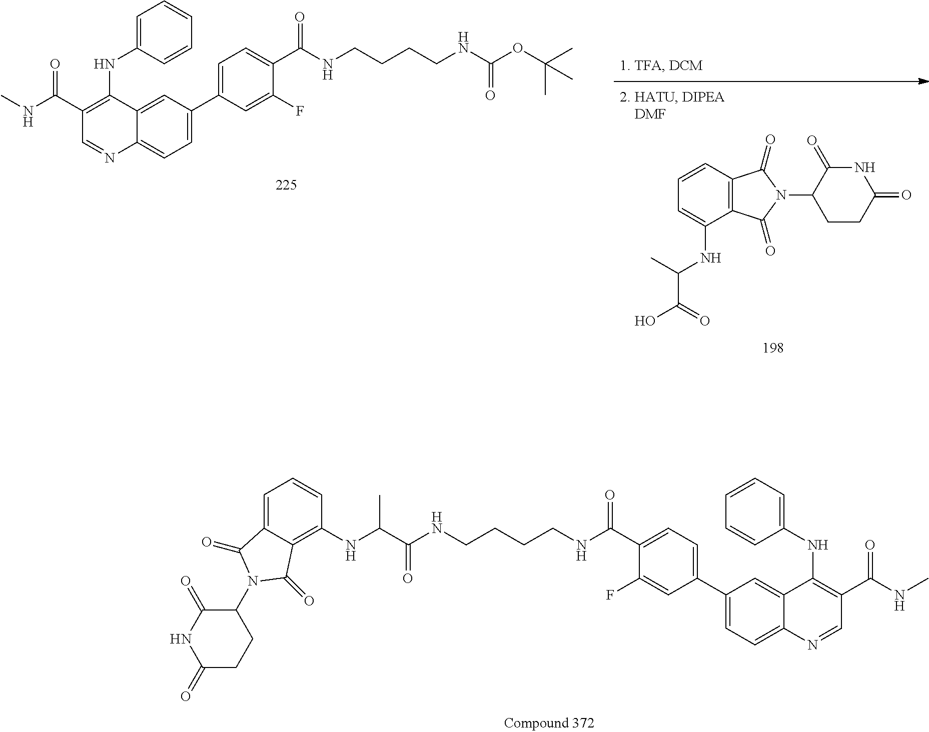 [SMILES: CNC(=O)c1cnc2ccc(-c3ccc(C(=O)NCCCCNC(=O)OC(C)(C)C)c(F)c3)cc2c1Nc1ccccc1 ; CC(Nc1cccc2c1C(=O)N(C1CCC(=O)NC1=O)C2=O)C(=O)O ; CNC(=O)c1cnc2ccc(-c3ccc(C(=O)NCCCCNC(=O)C(C)Nc4cccc5c4C(=O)N(C4CCC(=O)NC4=O)C5=O)c(F)c3)cc2c1Nc1ccccc1]

Into a 25 mL single-necked round-bottomed flask containing a well-stirred solution of tert-butyl N-[4-[[4-[4-anilino-3-(methylcarbamoyl)-6-quinolyl]-2-fluoro-benzoyl]amino]butyl]carbamate (225, 50 mg, 85.37 μmol) in anhydrous DCM (3 mL) was added TFA (48.67 mg, 426.86 μmol, 32.89 uL) under nitrogen atmosphere at room temperature. The resulting mixture was stirred at room temperature for 30 minutes. The reaction mixture was concentrated under reduced pressure to afford a crude residue which was dissolved in DMF (2 mL). DIPEA (33.10 mg, 256.12 μmol, 44.61 uL), 2-[[2-(2,6-dioxo-3-piperidyl)-1,3-dioxo-isoindolin-4-yl]amino]propanoic acid (198, 32.43 mg, 93.91 μmol) and HATU (48.69 mg, 128.06 μmol) were added under nitrogen atmosphere at room temperature. The resulting mixture was stirred at room temperature for 16 h. Ice cold water (20 mL) was added and stirred for 10 minutes. The resulting solid was filtered and dried to afford crude which was purified by prep-HPLC (SUNFIRE OBD C18(100×30)MM 50 Mobile phase: A: 0.1% TFA in water B: ACN to afford 4-anilino-6-[4-[4-[2-[[2-(2,6-dioxo-3-piperidyl)-1,3-dioxo-isoindolin-4-yl]amino]propanoylamino]butylcarbamoyl]-3-fluoro-phenyl]-N-methyl-quinoline-3-carboxamide (Compound 372, 15 mg, 17.90 μmol, 20.97% yield) as a yellow solid. ¹H NMR (400 MHz, DMSO-d₆) δ 11.11 (s, 1H), 8.82 (s, 1H), 8.52-8.44 (m, 1H), 8.41-8.33 (m, 2H), 8.28-8.22 (m, 1H), 8.06 (d, J=8.9 Hz, 1H), 7.75-7.58 (m, 4H), 7.43 (t, J=7.7 Hz, 2H), 7.34-7.22 (m, 3H), 7.07 (d, J=7.1 Hz, 1H), 6.93 (d, J=8.5 Hz, 1H), 6.74 (t, J=7.3 Hz, 1H), 5.06 (dd, J=12.9, 5.4 Hz, 1H), 4.23-4.15 (m, 1H), 3.28-3.22 (m, 2H), 3.16-3.09 (m, 2H), 2.94-2.81 (m, 1H), 2.37 (s, 3H), 2.08-1.98 (m, 1H), 1.49 (s, 4H), 1.38 (d, J=6.0 Hz, 3H). LCMS (ES+): m/z 813 [M+H]⁺

### Synthesis of Compound 373

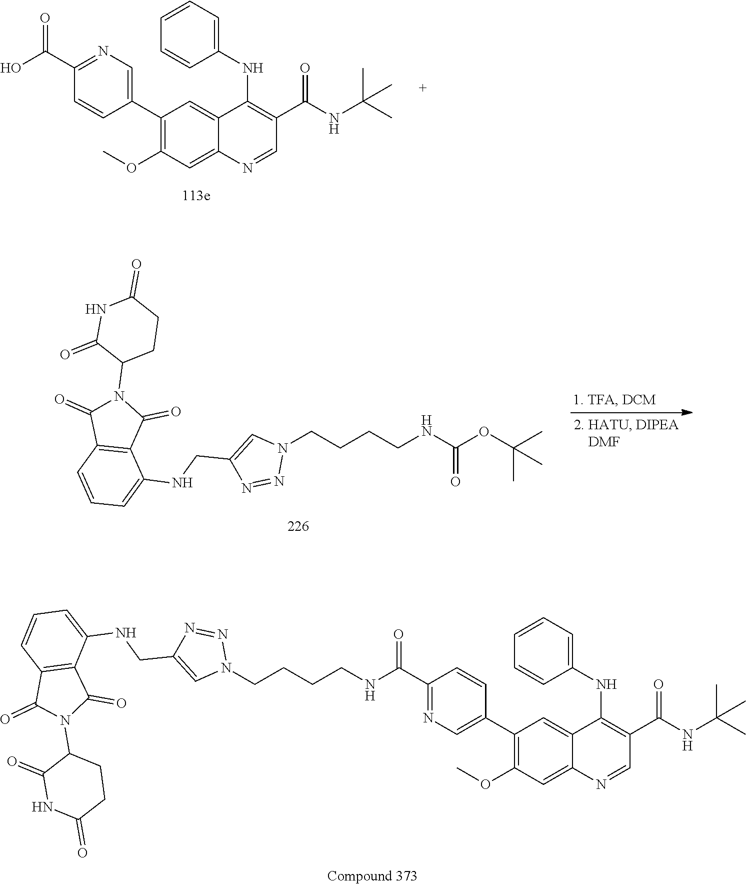 [SMILES: COc1cc2ncc(C(=O)NC(C)(C)C)c(Nc3ccccc3)c2cc1-c1ccc(C(=O)O)nc1 ; CC(C)(C)OC(=O)NCCCCn1cc(CNc2cccc3c2C(=O)N(C2CCC(=O)NC2=O)C3=O)nn1 ; COc1cc2ncc(C(=O)NC(C)(C)C)c(Nc3ccccc3)c2cc1-c1ccc(C(=O)NCCCCn2cc(CNc3cccc4c3C(=O)N(C3CCC(=O)NC3=O)C4=O)nn2)nc1]

Into a 25 mL single-necked round-bottomed flask containing a well-stirred solution of tert-butyl N-[4-[4-[[[2-(2,6-dioxo-3-piperidyl)-1,3-dioxo-isoindolin-4-yl]amino]methyl]triazol-1-yl]butyl]carbamate (226, 43.00 mg, 81.82 μmol) in anhydrous DCM (3 mL) was added TFA (42.41 mg, 371.93 μmol, 28.65 uL) under nitrogen atmosphere at room temperature. The resulting mixture was stirred at room temperature for 30 minutes. The reaction mixture was concentrated under reduced pressure to afford a crude residue which was dissolved in DMF (2 mL). DIPEA (28.84 mg, 223.16 μmol, 38.87 uL), 5-[4-anilino-3-(tert-butylcarbamoyl)-7-methoxy-6-quinolyl]pyridine-2-carboxylic acid (113e, 35 mg, 74.39 μmol) and HATU (42.43 mg, 111.58 μmol) were added. The resulting mixture was stirred at room temperature for 16 h. The reaction mixture was concentrated to afford crude residue which was purified by prep-HPLC (SUNFIRE OBD C18(100×30)MM 5μ) Mobile phase: A: 0.1% TFA in water B: ACN to afford N-(tert-butyl)-6-(6-((4-(4-(((2-(2,6-dioxopiperidin-3-yl)-1,3-dioxoisoindolin-4-yl)amino)methyl)-1H-1,2,3-triazol-1-yl)butyl)carbamoyl)pyridin-3-yl)-7-methoxy-4-(phenylamino)quinoline-3-carboxamide (Compound 373, 7 mg, 7.97 μmol, 10.72% yield) as a yellow solid. ¹H NMR (400 MHz, DMSO-d₆) δ 11.10 (s, 1H), 10.86 (s, 1H), 8.91 (t, J=6.3 Hz, 1H), 8.78 (t, J=1.5 Hz, 1H), 8.74 (s, 1H), 8.62 (s, 1H), 8.14 (d, J=2.1 Hz, 2H), 8.02 (d, J=4.4 Hz, 2H), 7.59-7.51 (m, 2H), 7.42 (t, J=7.8 Hz, 2H), 7.30-7.23 (m, 3H), 7.15 (d, J=8.6 Hz, 1H), 7.10-7.05 (m, 1H), 7.02 (d, J=7.0 Hz, 1H), 5.05 (dd, J=12.9, 5.3 Hz, 1H), 4.59 (s, 2H), 4.36 (t, J=7.1 Hz, 2H), 4.02 (s, 3H), 3.33 (q, J=6.7 Hz, 2H), 2.88 (ddd, J=18.4, 14.2, 5.5 Hz, 2H), 2.60 (s, 1H), 2.07-1.96 (m, 1H), 1.82 (q, J=7.9, 7.5 Hz, 2H), 1.50 (q, J=7.3 Hz, 2H), 1.04 (s, 9H). LCMS (ES+): m/z 878 [M+H]⁺

### Synthesis of Compound 374

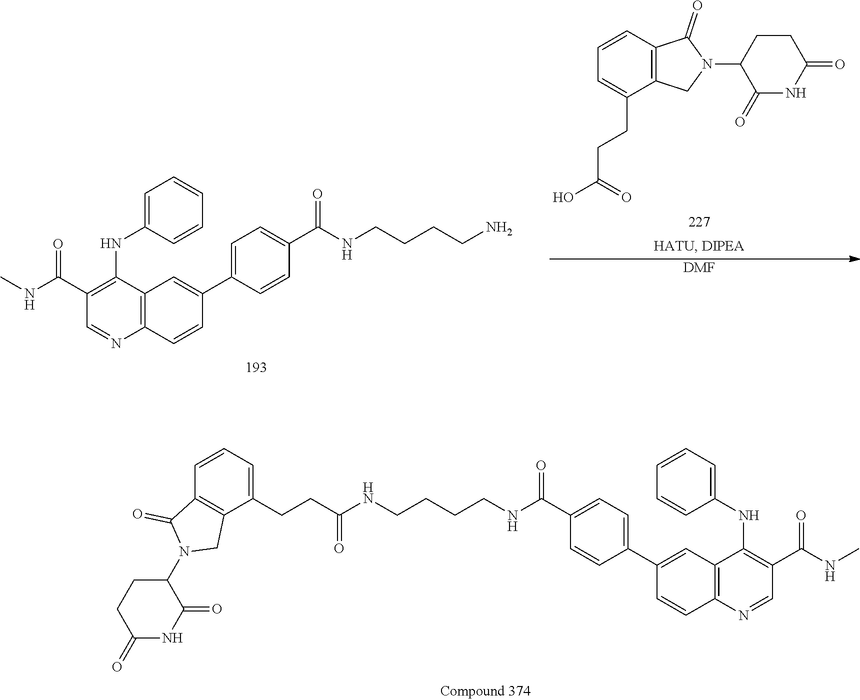

Into a 25 mL single-necked round-bottomed flask containing a well-stirred solution of 6-[4-(4-aminobutylcarbamoyl)phenyl]-4-anilino-N-methyl-quinoline-3-carboxamide (193, 147.82 mg, 316.15 μmol) and 3-[2-(2,6-dioxo-3-piperidyl)-1-oxo-isoindolin-4-yl]propanoic acid (227, 0.1 g, 316.15 μmol) in DMF (10 mL) were added DIPEA (122.58 mg, 948.44 μmol, 165.20 uL) and HATU (180.31 mg, 474.22 μmol) under nitrogen atmosphere at room temperature. The resulting mixture was stirred at room temperature for 16 h. After completion of reaction 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 4-anilino-6-[4-[4-[3-[2-(2,6-dioxo-3-piperidyl)-1-oxo-isoindolin-4-yl]propanoylamino]butylcarbamoyl]phenyl]-N-methyl-quinoline-3-carboxamide (Compound 374, 15 mg, 19.59 μmol, 6.20% yield) as a yellow colored solid. ¹H NMR (400 MHz, DMSO-d₆) δ 11.01 (s, 1H), 8.83 (s, 1H), 8.69 (s, 1H), 8.55 (d, J=5.8 Hz, 1H), 8.51 (d, J=4.7 Hz, 1H), 8.36 (s, 1H), 8.07 (d, J=8.8 Hz, 1H), 7.97 (d, J=8.1 Hz, 2H), 7.87 (t, J=5.7 Hz, 1H), 7.80 (d, J=8.0 Hz, 2H), 7.56 (t, J=4.3 Hz, 1H), 7.47-7.39 (m, 4H), 7.34-7.23 (m, 3H), 5.14 (dd, J=13.3, 5.1 Hz, 1H), 4.49 (d, J=17.2 Hz, 1H), 4.33 (d, J=17.1 Hz, 1H), 3.29-3.21 (m, 3H), 3.10-3.02 (m, 3H), 2.99-2.84 (m, 4H), 2.45-2.38 (m, 4H), 2.06-1.95 (m, 1H), 1.54-1.35 (m, 5H). LCMS (ES+): m/z 766 [M+H]⁺

### Synthesis of Compound 375

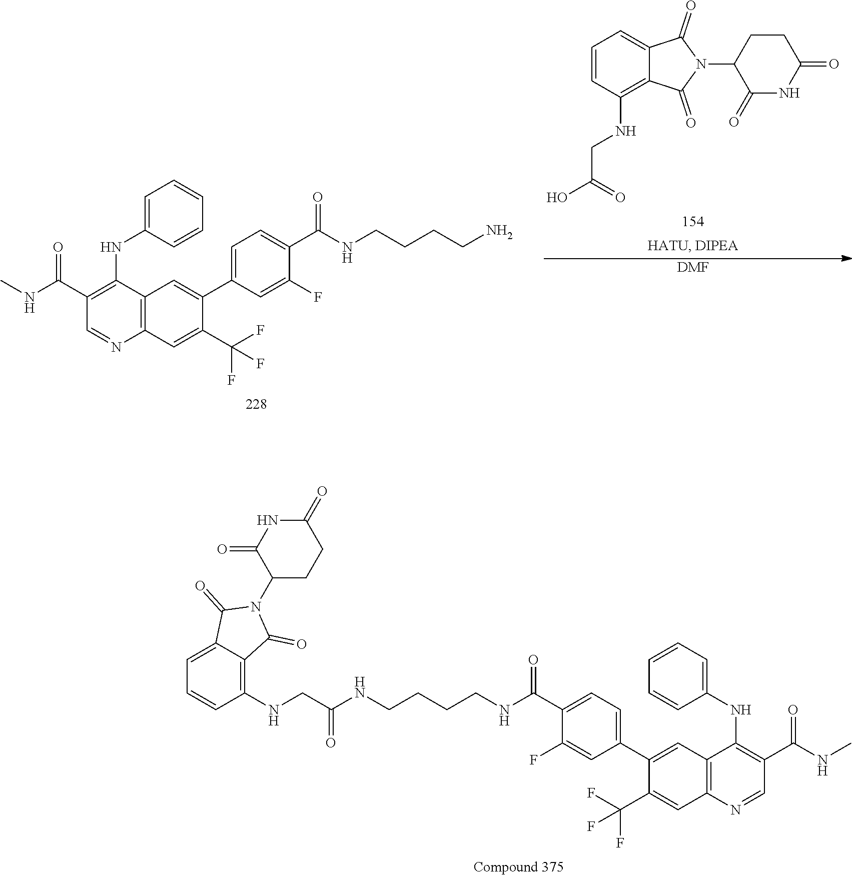

Into a 25 mL single-necked round-bottomed flask containing a well-stirred solution of 2-[[2-(2,6-dioxo-3-piperidyl)-1,3-dioxo-isoindolin-4-yl]amino]acetic acid (154, 50 mg, 150.93 μmol) and 6-[4-(4-aminobutylcarbamoyl)-3-fluoro-phenyl]-4-anilino-N-methyl-7-(trifluoromethyl)quinoline-3-carboxamide (228, 83.55 mg, 150.93 μmol) in DMF (5 mL) were added DIPEA (58.52 mg, 452.79 μmol, 78.87 uL) and HATU (86.08 mg, 226.39 μmol) under nitrogen atmosphere at room temperature. The resulting mixture was stirred at room temperature for 16 h. After completion of reaction, 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 6-(4-((4-(2-((2-(2,6-dioxopiperidin-3-yl)-1,3-dioxoisoindolin-4-yl)amino)acetamido)butyl)carbamoyl)-3-fluorophenyl)-N-methyl-4-(phenylamino)-7-(trifluoromethyl)quinoline-3-carboxamide (Compound 375, 25 mg, 28.84 μmol, 19.11% yield). ¹H NMR (400 MHz, DMSO-d₆) δ 11.11 (s, 1H), 10.78 (s, 1H), 8.94 (s, 1H), 8.51 (d, J=4.8 Hz, 1H), 8.45 (t, J=5.9 Hz, 1H), 8.41 (s, 1H), 8.14 (t, J=5.6 Hz, 1H), 7.66 (t, J=7.7 Hz, 1H), 7.59 (t, J=7.8 Hz, 1H), 7.36 (t, J=7.7 Hz, 2H), 7.31-7.18 (m, 3H), 7.14 (d, J=7.9 Hz, 2H), 7.06 (d, J=7.1 Hz, 1H), 6.96 (s, 1H), 6.86 (d, J=8.5 Hz, 1H), 5.07 (dd, J=12.9, 5.4 Hz, 1H), 3.93 (s, 3H), 3.26 (q, J=6.2 Hz, 2H), 3.14 (q, J=6.3 Hz, 2H), 2.89 (ddd, J=18.3, 14.2, 5.6 Hz, 2H), 2.60 (s, 1H), 2.38 (d, J=4.5 Hz, 3H), 2.08-1.96 (m, 2H), 1.58-1.44 (m, 4H). LCMS (ES+): m/z 867 [M+H]⁺

### Synthesis of Compound 376

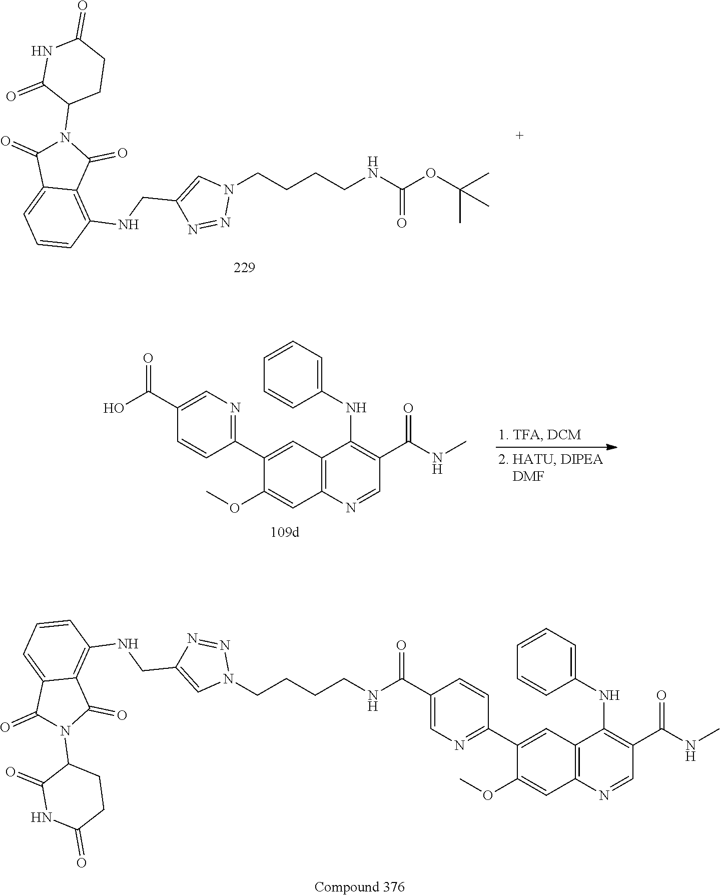 [SMILES: CC(C)(C)OC(=O)NCCCCn1cc(CNc2cccc3c2C(=O)N(C2CCC(=O)NC2=O)C3=O)nn1 ; CNC(=O)c1cnc2cc(OC)c(-c3ccc(C(=O)O)cn3)cc2c1Nc1ccccc1 ; CNC(=O)c1cnc2cc(OC)c(-c3ccc(C(=O)NCCCCn4cc(CNc5cccc6c5C(=O)N(C5CCC(=O)NC5=O)C6=O)nn4)cn3)cc2c1Nc1ccccc1]

To a stirred solution of tert-butyl N-[4-[4-[[[2-(2,6-dioxo-3-piperidyl)-1,3-dioxo-isoindolin-4-yl]amino]methyl]triazol-1-yl]butyl]carbamate (229, 61.33 mg, 116.70 μmol) in anhydrous Dichloromethane (5 mL) was added Trifluoroacetic acid (1.97 g, 17.26 mmol, 1.33 mL) at 0° C. Then reaction mixture was stirred at room temperature for 3 hours. The reaction mixture was concentrated completely and this crude was taken as such for the next step. To the stirred solution of above crude in N,N-Dimethylformamide (6 mL) was added 6-[4-anilino-7-methoxy-3-(methylcarbamoyl)-6-quinolyl]pyridine-3-carboxylic acid (109d, 50 mg, 116.70 μmol) followed by HATU (66.56 mg, 175.05 μmol) and N,N-Diisopropylethylamine (75.41 mg, 583.51 μmol, 101.64 uL). The reaction mixture was stirred at room temperature for 16 hours. Ice cold water (15 mL) was added and stirred for 10 minutes, and then extracted with ethyl acetate. Then combined organics were dried over anhydrous sodium sulfate and then concentrated to yield the crude product which was purified by prep-HPLC(SUNFIRE OBD C18(100×30)MM 5μ) Mobile phase: A: 0.1% TFA in water B: ACN) to yield 6-(5-((4-(4-(((2-(2,6-dioxopiperidin-3-yl)-1,3-dioxoisoindolin-4-yl)amino)methyl)-1H-1,2,3-triazol-1-yl)butyl)carbamoyl)pyridin-2-yl)-7-methoxy-N-methyl-4-(phenylamino)quinoline-3-carboxamide (Compound 376, 7.0 mg, 7.03 μmol, 6.03% yield) as light yellow colored solid. ¹H NMR (400 MHz, Methanol-d₄) δ 8.96 (d, J=2.3 Hz, 1H), 8.80 (dd, J=4.4, 1.4 Hz, 1H), 8.68 (s, 1H), 8.57 (s, 1H), 8.54 (dd, J=8.4, 1.4 Hz, 1H), 8.20 (dd, J=8.2, 2.3 Hz, 1H), 7.95 (s, 1H), 7.89 (d, J=8.3 Hz, 1H), 7.59 (dd, J=8.4, 4.5 Hz, 1H), 7.53-7.47 (m, 1H), 7.45 (d, J=7.6 Hz, 2H), 7.38 (t, J=3.5 Hz, 2H), 7.33 (d, J=7.6 Hz, 2H), 7.08 (d, J=8.5 Hz, 1H), 7.02 (d, J=7.2 Hz, 1H), 6.43 (d, J=0.7 Hz, 2H), 5.03 (dd, J=12.6, 5.4 Hz, 1H), 4.64 (s, 2H), 4.46 (t, J=6.9 Hz, 2H), 4.10 (s, 3H), 3.41 (t, J=7.0 Hz, 2H), 2.89-2.75 (m, 2H), 2.74-2.65 (m, 2H), 2.62 (s, 3H), 2.12-2.04 (m, 1H), 1.97 (q, J=7.7 Hz, 2H), 1.61 (q, J=7.4 Hz, 2H). LCMS (ES+): m/z 836 [M+H]⁺

### Synthesis of Compound 378

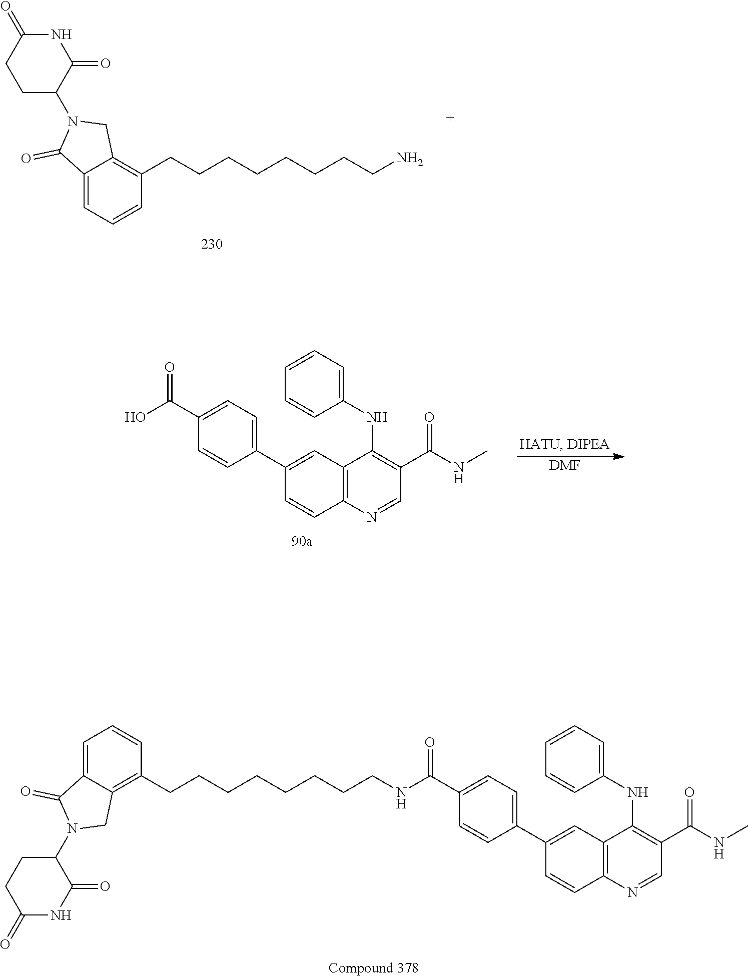

Into a 25 mL single-necked round-bottomed flask containing a well-stirred solution of 3-[4-(8-aminooctyl)-1-oxo-isoindolin-2-yl]piperidine-2,6-dione (230, 0.2 g, 538.40 μmol) and 4-[4-anilino-3-(methylcarbamoyl)-6-quinolyl]benzoic acid (90a, 235.37 mg, 592.24 μmol) in DMF (10 mL) were added DIPEA (208.75 mg, 1.62 mmol, 281.34 uL) and HATU (307.07 mg, 807.60 μmol) under nitrogen atmosphere at room temperature. The resulting mixture was stirred at room temperature for 16 h. After completion of reaction 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 4-anilino-6-[4-[8-[2-(2,6-dioxo-3-piperidyl)-1-oxo-isoindolin-4-yl]octylcarbamoyl]phenyl]-N-methyl-quinoline-3-carboxamide (Compound 378, 75 mg, 99.88 μmol, 18.55% yield). ¹H NMR (400 MHz, DMSO-d₆) δ 11.33 (s, 1H), 10.99 (d, J=5.3 Hz, 1H), 8.81 (d, J=14.3 Hz, 2H), 8.56 (t, J=5.8 Hz, 1H), 8.51-8.45 (m, 1H), 8.44-8.39 (m, 1H), 8.11-8.04 (m, 1H), 8.01-7.94 (m, 2H), 7.83 (d, J=7.8 Hz, 2H), 7.58-7.53 (m, 1H), 7.50-7.41 (m, 4H), 7.35 (t, J=7.1 Hz, 1H), 7.30 (d, J=7.6 Hz, 2H), 5.13 (dd, J=13.3, 5.4 Hz, 1H), 4.45 (dd, J=17.2, 5.5 Hz, 1H), 4.35-4.22 (m, 1H), 3.31-3.21 (m, 2H), 2.99-2.84 (m, 1H), 2.69-2.58 (m, 4H), 2.33 (dd, J=6.2, 3.7 Hz, 3H), 2.00 (s, 1H), 1.56 (d, J=27.4 Hz, 5H), 1.31 (s, 9H). LCMS (ES+): m/z 751 [M+H]

### Synthesis of Compound 379

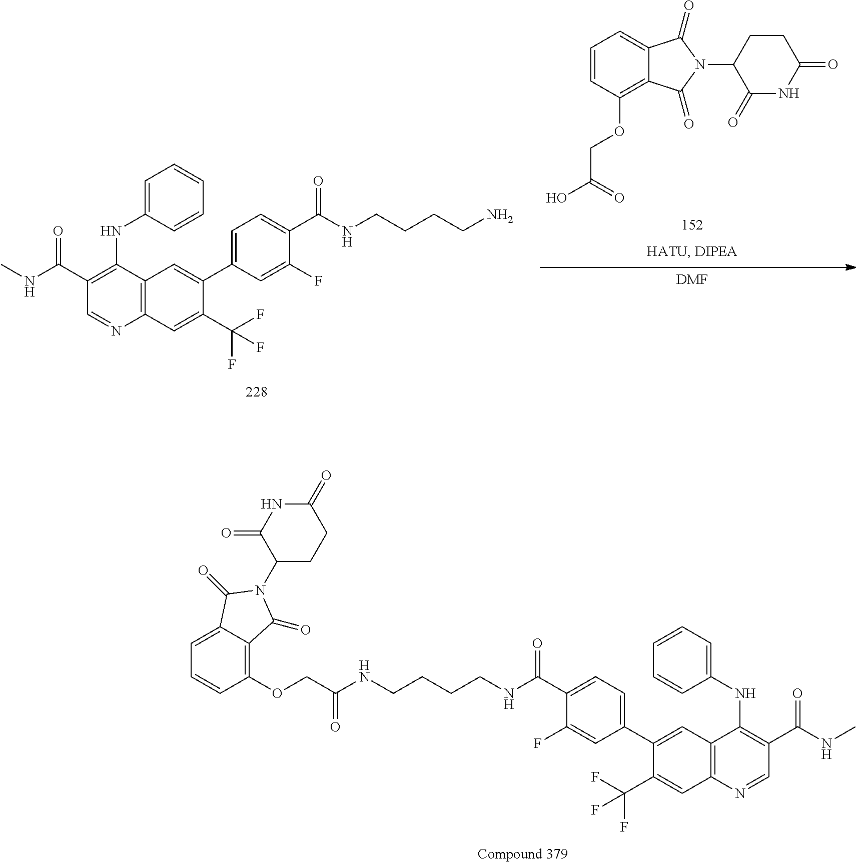

Into a 25 mL single-necked round-bottomed flask containing a well-stirred solution of 6-[4-(4-aminobutylcarbamoyl)-3-fluoro-phenyl]-4-anilino-N-methyl-7-(trifluoromethyl)quinoline-3-carboxamide (228, 83.30 mg, 150.48 μmol) and 2-[2-(2,6-dioxo-3-piperidyl)-1,3-dioxo-isoindolin-4-yl]oxyacetic acid (152, 50 mg, 150.48 μmol) in DMF (5 mL) were added DIPEA (58.35 mg, 451.45 μmol, 78.63 uL) and HATU (85.83 mg, 225.72 μmol) under nitrogen atmosphere at room temperature. The resulting mixture was stirred at room temperature for 16 h. After completion of reaction 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 6-(4-((4-(2-((2-(2,6-dioxopiperidin-3-yl)-1,3-dioxoisoindolin-4-yl)oxy)acetamido)butyl)carbamoyl)-3-fluorophenyl)-N-methyl-4-(phenylamino)-7-(trifluoromethyl)quinoline-3-carboxamide (Compound 379, 25 mg, 28.81 μmol, 19.14% yield). ¹H NMR (400 MHz, DMSO-d₆) δ 11.12 (s, 1H), 10.54 (s, 1H), 8.93 (s, 1H), 8.52 (d, J=4.8 Hz, 1H), 8.49-8.41 (m, 1H), 8.39 (s, 1H), 8.31 (s, 1H), 8.01 (t, J=5.8 Hz, 1H), 7.81 (dd, J=8.5, 7.3 Hz, 1H), 7.65 (t, J=7.6 Hz, 1H), 7.49 (d, J=7.2 Hz, 1H), 7.39 (d, J=8.6 Hz, 1H), 7.33 (t, J=7.7 Hz, 2H), 7.24 (dd, J=14.1, 9.5 Hz, 1H), 7.16 (t, J=8.0 Hz, 1H), 7.11 (d, J=7.8 Hz, 2H), 5.12 (dd, J=12.8, 5.5 Hz, 1H), 4.78 (s, 2H), 3.30-3.23 (m, 2H), 3.22-3.15 (m, 2H), 2.96-2.82 (m, 2H), 2.62-2.56 (m, 2H), 2.42 (d, J=4.5 Hz, 3H), 2.08-1.98 (m, 1H), 1.52 (s, 4H). LCMS (ES+): m/z 868 [M+H]⁺

### Synthesis of Compound 380

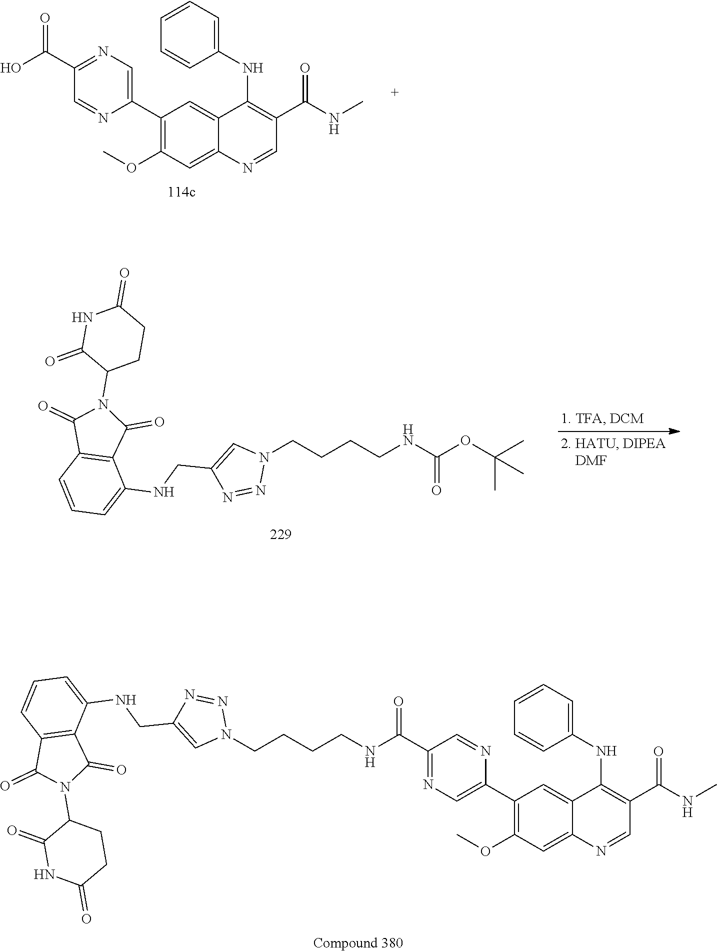 [SMILES: CNC(=O)c1cnc2cc(OC)c(-c3cnc(C(=O)O)cn3)cc2c1Nc1ccccc1 ; CC(C)(C)OC(=O)NCCCCn1cc(CNc2cccc3c2C(=O)N(C2CCC(=O)NC2=O)C3=O)nn1 ; CNC(=O)c1cnc2cc(OC)c(-c3cnc(C(=O)NCCCCn4cc(CNc5cccc6c5C(=O)N(C5CCC(=O)NC5=O)C6=O)nn4)cn3)cc2c1Nc1ccccc1]

Into a 25 mL single-necked round-bottomed flask containing a well-stirred solution of tert-butyl N-[4-[4-[[[2-(2,6-dioxo-3-piperidyl)-1,3-dioxo-isoindolin-4-yl]amino]methyl]triazol-1-yl]butyl]carbamate (229, 121.16 mg, 230.54 μmol) in anhydrous DCM (4 mL) was added TFA (119.48 mg, 1.05 mmol, 80.73 uL) under nitrogen atmosphere at room temperature. The resulting mixture was stirred at room temperature for 30 minutes. The reaction mixture was concentrated under reduced pressure to afford a crude residue which was dissolved in DMF (3 mL). DIPEA (81.26 mg, 628.74 μmol, 109.51 uL), 5-[4-anilino-7-methoxy-3-(methylcarbamoyl)-6-quinolyl]pyrazine-2-carboxylic acid (114c, 90 mg, 209.58 μmol) and HATU (119.53 mg, 314.37 μmol) were added under nitrogen atmosphere at room temperature. The resulting mixture was stirred at room temperature for 16 h. The reaction mixture was concentrated to afford crude residue which was purified by prep-HPLC (SUNFIRE OBD C18(100×30)MM 5μ) Mobile phase: A: 0.1% TFA in water B: ACN to afford 6-(5-((4-(4-(((2-(2,6-dioxopiperidin-3-yl)-1,3-dioxoisoindolin-4-yl)amino)methyl)-1H-1,2,3-triazol-1-yl)butyl)carbamoyl)pyrazin-2-yl)-7-methoxy-N-methyl-4-(phenylamino)quinoline-3-carboxamide (Compound 380, 45 mg, 51.94 μmol, 24.78% yield) as a yellow solid. ¹H NMR (400 MHz, DMSO-d₆) δ 11.43 (s, 1H), 11.10 (s, 1H), 9.20 (d, J=1.4 Hz, 1H), 9.14 (d, J=1.5 Hz, 1H), 9.02 (t, J=6.1 Hz, 1H), 8.83 (s, 1H), 8.80 (s, 1H), 8.56-8.48 (m, 1H), 8.03 (s, 1H), 7.56 (dd, J=8.6, 7.2 Hz, 1H), 7.52 (s, 1H), 7.40 (t, J=7.6 Hz, 2H), 7.34-7.25 (m, 1H), 7.25 (d, J=7.9 Hz, 2H), 7.15 (d, J=8.6 Hz, 1H), 7.07 (t, J=6.2 Hz, 1H), 7.02 (d, J=7.1 Hz, 1H), 5.05 (dd, J=12.8, 5.3 Hz, 1H), 4.59 (d, J=5.8 Hz, 2H), 4.36 (t, J=7.0 Hz, 2H), 4.07 (s, 3H), 3.38-3.30 (m, 2H), 2.93-2.82 (m, 1H), 2.62-2.55 (m, 2H), 2.39 (d, J=4.6 Hz, 3H), 2.07-1.96 (m, 1H), 1.83 (p, J=7.0 Hz, 2H), 1.57-1.44 (m, 2H). LCMS (ES+): m/z 837 [M+H]⁺

### Synthesis of Compound 381

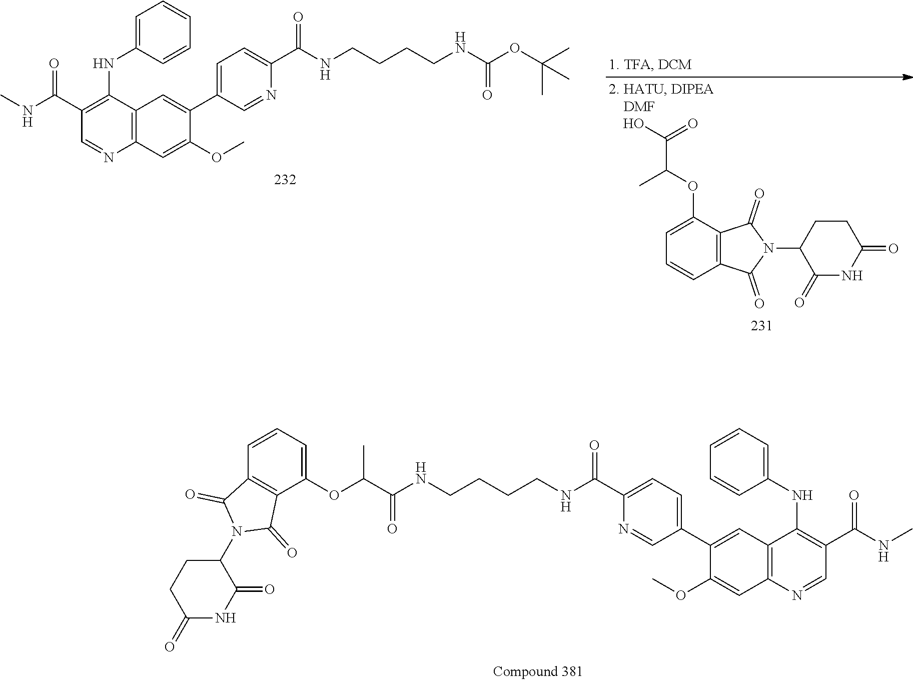 [SMILES: CNC(=O)c1cnc2cc(OC)c(-c3ccc(C(=O)NCCCCNC(=O)OC(C)(C)C)nc3)cc2c1Nc1ccccc1 ; CC(Oc1cccc2c1C(=O)N(C1CCC(=O)NC1=O)C2=O)C(=O)O ; CNC(=O)c1cnc2cc(OC)c(-c3ccc(C(=O)NCCCCNC(=O)C(C)Oc4cccc5c4C(=O)N(C4CCC(=O)NC4=O)C5=O)nc3)cc2c1Nc1ccccc1]

An oven dried round bottom flask was charged with a solution of tert-butyl N-[4-[[5-[4-anilino-7-methoxy-3-(methylcarbamoyl)-6-quinolyl]pyridine-2-carbonyl]amino]butyl]carbamate (232, 133.12 mg, 222.36 μmol) in Dichloromethane (3 mL), Trifluoroacetic acid (2.96 g, 25.96 mmol, 2 mL) was added at room temperature. The reaction mixture was stirred for an hour at room temperature and the reaction mixture was concentrated under reduced pressure. To a solution of crude product in DMF (3 mL), 2-[2-(2,6-dioxo-3-piperidyl)-1,3-dioxo-isoindolin-4-yl]oxypropanoic acid (231, 70 mg, 202.14 μmol), DIPEA (130.62 mg, 1.01 mmol, 176.04 uL) and HATU (92.23 mg, 242.57 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 6-(6-((4-(2-((2-(2,6-dioxopiperidin-3-yl)-1,3-dioxoisoindolin-4-yl)oxy)propanamido)butyl)carbamoyl)pyridin-3-yl)-7-methoxy-N-methyl-4-(phenylamino)quinoline-3-carboxamide (Compound 381, 46 mg, 52.69 μmol, 26.07% yield) as yellow solid. ¹H NMR (400 MHz, DMSO-d₆) δ 11.22 (s, 1H), 11.13 (s, 1H), 8.84 (t, J=6.1 Hz, 1H), 8.80 (s, 1H), 8.73 (d, J=2.1 Hz, 1H), 8.56-8.50 (m, 1H), 8.45 (s, 1H), 8.17-8.11 (m, 2H), 8.08 (dd, J=8.1, 2.2 Hz, 1H), 7.83-7.76 (m, 1H), 7.50 (s, 1H), 7.44 (dd, J=14.2, 7.3 Hz, 3H), 7.37-7.30 (m, 2H), 7.26 (d, J=7.8 Hz, 2H), 5.11 (dd, J=13.0, 5.4 Hz, 1H), 4.95 (q, J=6.5 Hz, 1H), 4.02 (s, 3H), 3.37-3.26 (m, 2H), 3.20-3.09 (m, 3H), 2.96-2.82 (m, 1H), 2.63-2.57 (m, 2H), 2.37 (d, J=4.5 Hz, 3H), 2.07-1.98 (m, 1H), 1.52 (d, J=6.7 Hz, 4H), 1.49-1.41 (m, 2H). LCMS (ES+): m/z 827 [M+H]⁺

### Synthesis of Compound 382

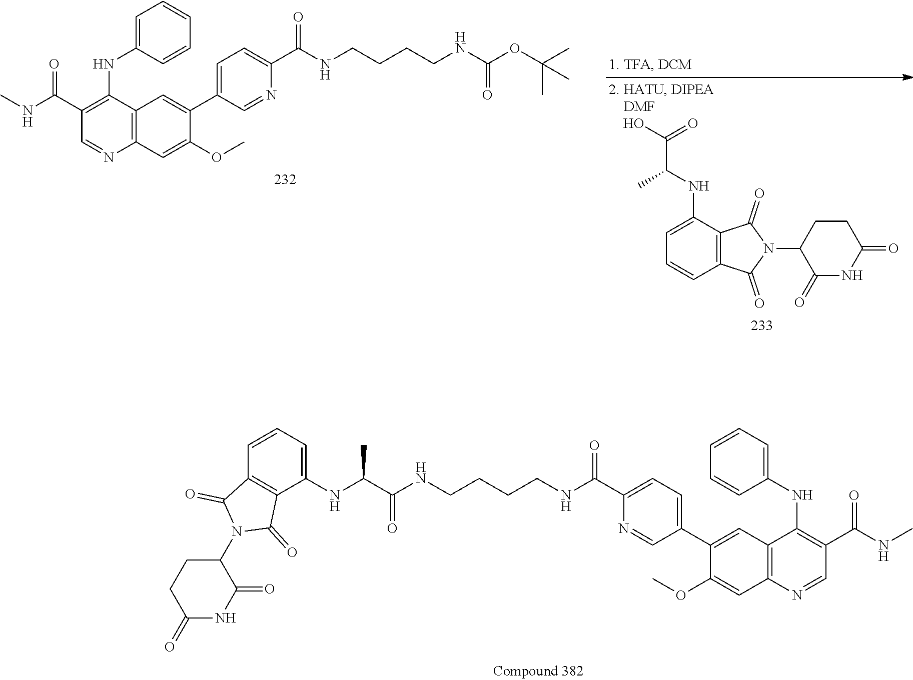 [SMILES: CNC(=O)c1cnc2cc(OC)c(-c3ccc(C(=O)NCCCCNC(=O)OC(C)(C)C)nc3)cc2c1Nc1ccccc1 ; C[C@@H](Nc1cccc2c1C(=O)N(C1CCC(=O)NC1=O)C2=O)C(=O)O ; CNC(=O)c1cnc2cc(OC)c(-c3ccc(C(=O)NCCCCNC(=O)[C@H](C)Nc4cccc5c4C(=O)N(C4CCC(=O)NC4=O)C5=O)nc3)cc2c1Nc1ccccc1]

An oven dried round bottom flask was charged with a solution of tert-butyl N-[4-[[5-[4-anilino-7-methoxy-3-(methylcarbamoyl)-6-quinolyl]pyridine-2-carbonyl]amino]butyl]carbamate (232, 133.50 mg, 222.99 μmol) in Dichloromethane (3 mL). Trifluoroacetic acid (2.96 g, 25.96 mmol, 2 mL) was added at room temperature. The reaction mixture was stirred for an hour at room temperature and the reaction mixture was concentrated under reduced pressure. To a solution of crude product in DMF (3 mL), (2R)-2-[[2-(2,6-dioxo-3-piperidyl)-1,3-dioxo-isoindolin-4-yl]amino]propanoic acid (233, 70 mg, 202.72 μmol), DIPEA (131.00 mg, 1.01 mmol, 176.55 uL) and HATU (92.50 mg, 243.26 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 6-(6-((4-((2R)-2-((2-(2,6-dioxopiperidin-3-yl)-1,3-dioxoisoindolin-4-yl)amino)propanamido)butyl)carbamoyl)pyridin-3-yl)-7-methoxy-N-methyl-4-(phenylamino)quinoline-3-carboxamide (Compound 382, 12.74 mg, 15.31 μmol, 7.55% yield) as yellow solid. ¹H NMR (400 MHz, DMSO-d₆) δ 11.17 (s, 1H), 11.11 (s, 1H), 8.83 (t, J=6.1 Hz, 1H), 8.79 (s, 1H), 8.71 (d, J=2.1 Hz, 1H), 8.56-8.48 (m, 1H), 8.41 (s, 1H), 8.23 (q, J=4.7 Hz, 1H), 8.15-8.03 (m, 2H), 7.60 (dd, J=8.5, 7.1 Hz, 1H), 7.49 (s, 1H), 7.42 (t, J=7.7 Hz, 2H), 7.30 (t, J=7.4 Hz, 1H), 7.24 (d, J=7.8 Hz, 2H), 7.05 (d, J=7.0 Hz, 1H), 6.95-6.89 (m, 1H), 6.72 (t, J=7.2 Hz, 1H), 5.06 (dd, J=12.9, 5.4 Hz, 1H), 4.18 (td, J=7.1, 2.5 Hz, 1H), 4.01 (s, 3H), 3.36-3.26 (m, 2H), 3.12 (d, J=6.7 Hz, 2H), 2.88 (ddd, J=17.3, 14.0, 5.4 Hz, 1H), 2.63-2.55 (m, 2H), 2.38 (d, J=4.5 Hz, 3H), 2.08-1.96 (m, 1H), 1.58-1.42 (m, 4H), 1.37 (d, J=6.8 Hz, 2H). LCMS (ES+): m/z 826 [M+H]⁺

### Synthesis of Compound 383

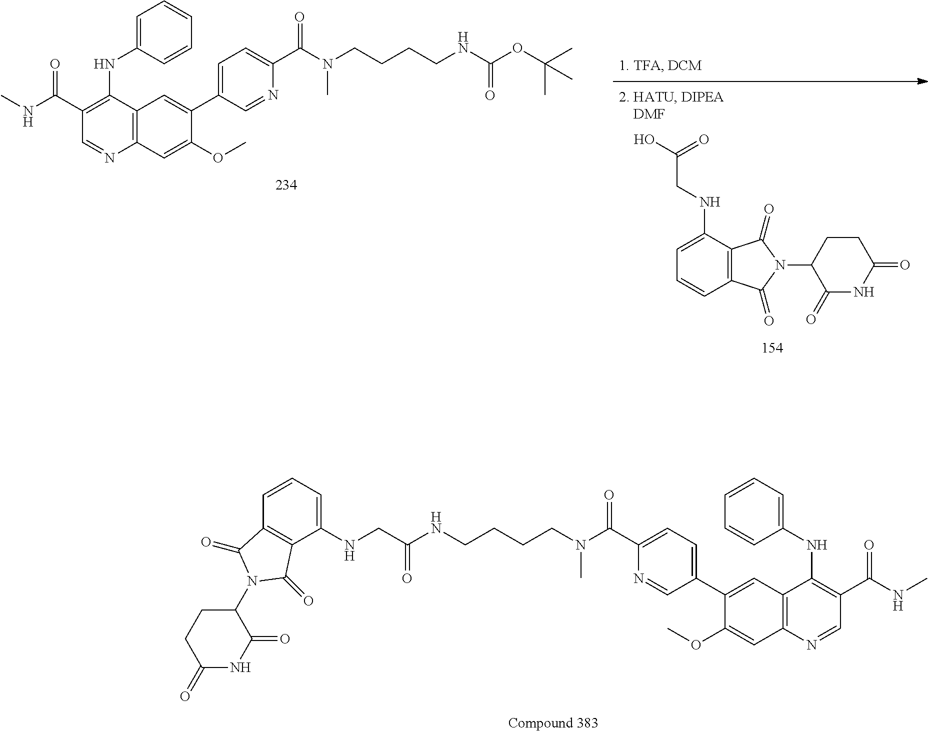 [SMILES: CNC(=O)c1cnc2cc(OC)c(-c3ccc(C(=O)N(C)CCCCNC(=O)OC(C)(C)C)nc3)cc2c1Nc1ccccc1 ; O=C(O)CNc1cccc2c1C(=O)N(C1CCC(=O)NC1=O)C2=O ; CNC(=O)c1cnc2cc(OC)c(-c3ccc(C(=O)N(C)CCCCNC(=O)CNc4cccc5c4C(=O)N(C4CCC(=O)NC4=O)C5=O)nc3)cc2c1Nc1ccccc1]

An oven dried round bottom flask was charged with a solution of tert-butyl N-[4-[[5-[4-anilino-7-methoxy-3-(methylcarbamoyl)-6-quinolyl]pyridine-2-carbonyl]-methyl-amino]butyl]carbamate (234, 122.07 mg, 199.23 μmol) in Dichloromethane (3 mL), Trifluoroacetic acid (2.96 g, 25.96 mmol, 2 mL) was added at room temperature. The reaction mixture was stirred for an hour at room temperature and the reaction mixture was concentrated under reduced pressure. To a solution of crude product in DMF (3 mL), 2-[[2-(2,6-dioxo-3-piperidyl)-1,3-dioxo-isoindolin-4-yl]amino]acetic acid (154, 60 mg, 181.12 μmol), DIPEA (117.04 mg, 905.58 μmol, 157.73 uL) and HATU (82.64 mg, 217.34 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 6-(6-((4-(2-((2-(2,6-dioxopiperidin-3-yl)-1,3-dioxoisoindolin-4-yl)amino)acetamido)butyl)(methyl)carbamoyl)pyridin-3-yl)-7-methoxy-N-methyl-4-(phenylamino)quinoline-3-carboxamide (Compound 383, 23.86 mg, 28.02 μmol, 15.47% yield) as yellow solid. ¹H NMR (400 MHz, DMSO-d₆) δ 11.18 (d, J=11.1 Hz, 1H), 11.11 (s, 1H), 8.78 (s, 1H), 8.63 (d, J=18.2 Hz, 1H), 8.51 (d, J=5.0 Hz, 1H), 8.41 (d, J=16.4 Hz, 1H), 8.13 (dt, J=30.5, 5.6 Hz, 1H), 8.05-8.01 (m, 1H), 7.63 (d, J=8.1 Hz, 1H), 7.62-7.51 (m, 1H), 7.48 (dd, J=10.9, 2.0 Hz, 1H), 7.45-7.38 (m, 2H), 7.34-7.27 (m, 1H), 7.24 (d, J=7.8 Hz, 2H), 7.05 (dd, J=11.6, 7.0 Hz, 1H), 6.94 (dt, J=20.0, 5.7 Hz, 1H), 6.84 (dd, J=23.0, 8.6 Hz, 1H), 5.06 (dt, J=11.7, 5.4 Hz, 1H), 4.01 (d, J=8.0 Hz, 3H), 3.94 (d, J=5.0 Hz, 1H), 3.88 (d, J=5.3 Hz, 1H), 3.47 (t, J=7.1 Hz, 1H), 3.30 (t, J=7.5 Hz, 1H), 3.17 (q, J=6.4 Hz, 1H), 3.07-3.01 (m, 1H), 2.98 (s, 1H), 2.93 (s, 1H), 2.89-2.82 (m, 1H), 2.62-2.55 (m, 1H), 2.36 (t, J=4.6 Hz, 3H), 2.06-1.97 (m, 1H), 1.64-1.52 (m, 2H), 1.52-1.43 (m, 1H), 1.33-1.21 (m, 1H). LCMS (ES+): m/z 826 [M+H]⁺

### Synthesis of Compound 384

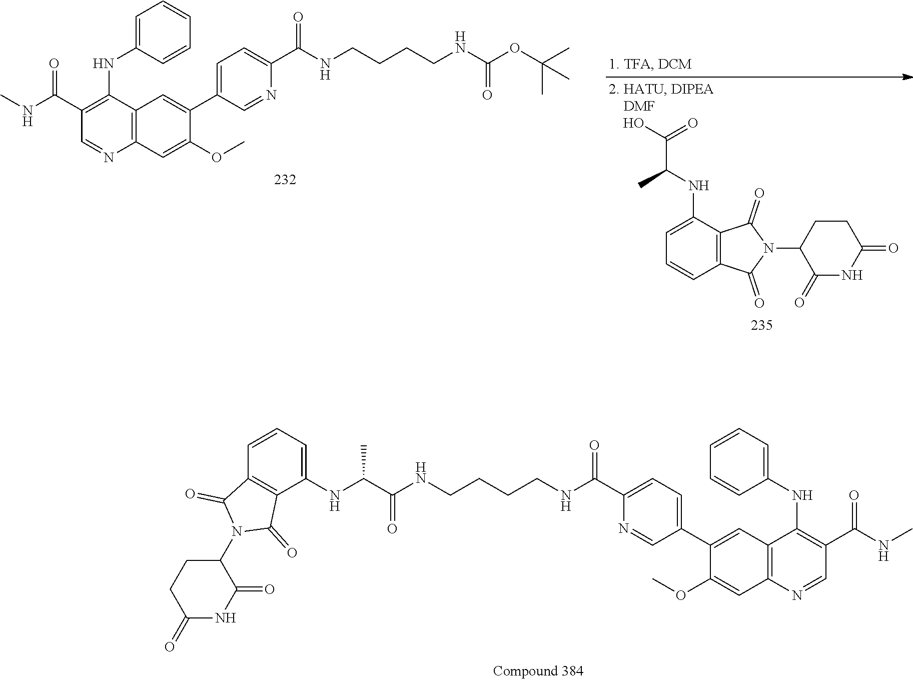 [SMILES: CNC(=O)c1cnc2cc(OC)c(-c3ccc(C(=O)NCCCCNC(=O)OC(C)(C)C)nc3)cc2c1Nc1ccccc1 ; C[C@H](Nc1cccc2c1C(=O)N(C1CCC(=O)NC1=O)C2=O)C(=O)O ; CNC(=O)c1cnc2cc(OC)c(-c3ccc(C(=O)NCCCCNC(=O)[C@@H](C)Nc4cccc5c4C(=O)N(C4CCC(=O)NC4=O)C5=O)nc3)cc2c1Nc1ccccc1]

An oven dried round bottom flask was charged with a solution of tert-butyl N-[4-[[5-[4-anilino-7-methoxy-3-(methylcarbamoyl)-6-quinolyl]pyridine-2-carbonyl]amino]butyl]carbamate (232, 133.50 mg, 222.99 μmol) in Dichloromethane (3 mL), Trifluoroacetic acid (2.96 g, 25.96 mmol, 2 mL) was added at room temperature. The reaction mixture was stirred for an hour at room temperature and the reaction mixture was concentrated under reduced pressure. To a solution of crude product in DMF (3 mL), (2S)-2-[[2-(2,6-dioxo-3-piperidyl)-1,3-dioxo-isoindolin-4-yl]amino]propanoic acid (235, 70.00 mg, 202.72 μmol), DIPEA (131.00 mg, 1.01 mmol, 176.55 uL) and HATU (92.50 mg, 243.26 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 6-(6-((4-((2S)-2-((2-(2,6-dioxopiperidin-3-yl)-1,3-dioxoisoindolin-4-yl)amino)propanamido)butyl)carbamoyl)pyridin-3-yl)-7-methoxy-N-methyl-4-(phenylamino)quinoline-3-carboxamide (Compound 384, 11.80 mg, 14.07 μmol, 6.94% yield) as yellow solid. ¹H NMR (400 MHz, DMSO-d₆) δ 11.21 (s, 1H), 11.11 (s, 1H), 8.83 (t, J=6.1 Hz, 1H), 8.79 (s, 1H), 8.71 (d, J=2.1 Hz, 1H), 8.56-8.49 (m, 1H), 8.43 (s, 1H), 8.26-8.20 (m, 1H), 8.14-8.05 (m, 2H), 7.60 (dd, J=8.5, 7.1 Hz, 1H), 7.50 (s, 1H), 7.42 (t, J=7.6 Hz, 2H), 7.31 (t, J=7.4 Hz, 1H), 7.25 (d, J=7.8 Hz, 2H), 7.05 (d, J=7.1 Hz, 1H), 6.92 (d, J=8.7 Hz, 1H), 6.72 (t, J=7.2 Hz, 1H), 5.06 (dd, J=12.9, 5.4 Hz, 1H), 4.23-4.14 (m, 1H), 4.01 (s, 3H), 3.96 (s, 1H), 3.31 (q, J=6.6 Hz, 2H), 3.16-3.07 (m, 2H), 2.88 (ddd, J=17.1, 13.9, 5.4 Hz, 1H), 2.62-2.55 (m, 2H), 2.37 (d, J=4.5 Hz, 3H), 2.06-1.96 (m, 1H), 1.59-1.49 (m, 2H), 1.47-1.40 (m, 2H), 1.37 (d, J=6.6 Hz, 3H). LCMS (ES+): m/z 826 [M+H]⁺

### Synthesis of Compound 385

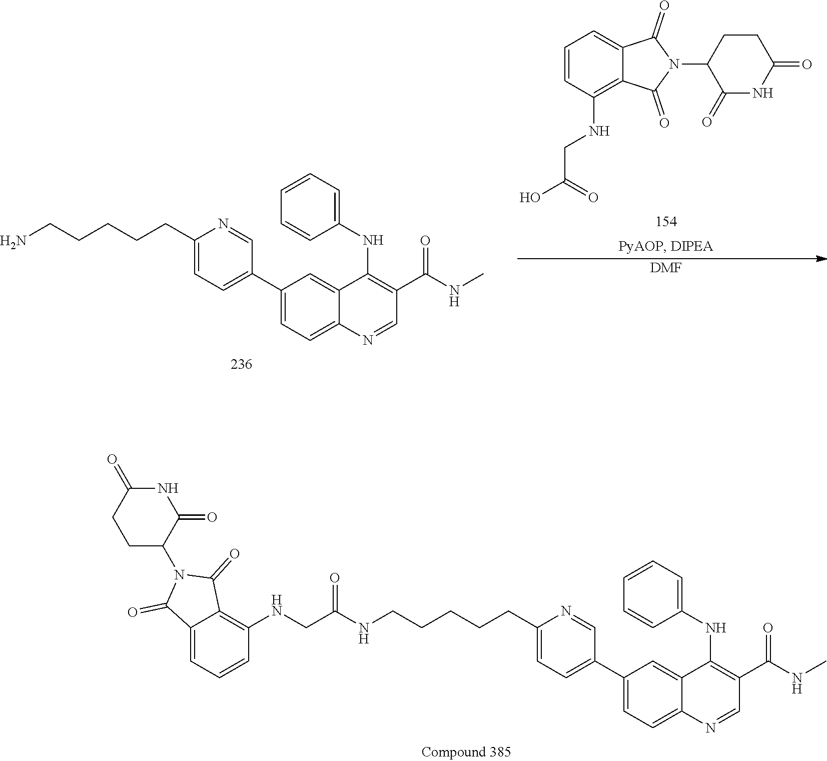

To a stirred solution of 6-[6-(5-aminopentyl)-3-pyridyl]-4-anilino-N-methyl-quinoline-3-carboxamide (236, 50 mg, 113.75 μmol), 2-[[2-(2,6-dioxo-3-piperidyl)-1,3-dioxo-isoindolin-4-yl]amino]acetic acid (154, 37.68 mg, 113.75 μmol) in DMF (5 mL) and DCM (5 mL) was added DIPEA (73.51 mg, 568.76 μmol, 99.07 uL) and PyAOP (88.96 mg, 170.63 μmol). The reaction mixture was stirred for 16 hr at 25° C. The reaction mixture was diluted with water (10 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield pure product 4-anilino-6-[6-[5-[[2-[[2-(2,6-dioxo-3-piperidyl)-1,3-dioxo-isoindolin-4-yl]amino]acetyl]amino]pentyl]-3-pyridyl]-N-methyl-quinoline-3-carboxamide (Compound 385, 3 mg, 3.79 μmol, 3.33% yield) as a light yellow solid. LCMS (ES+): m/z 753 [M+H]⁺

### Synthesis of Compound 386

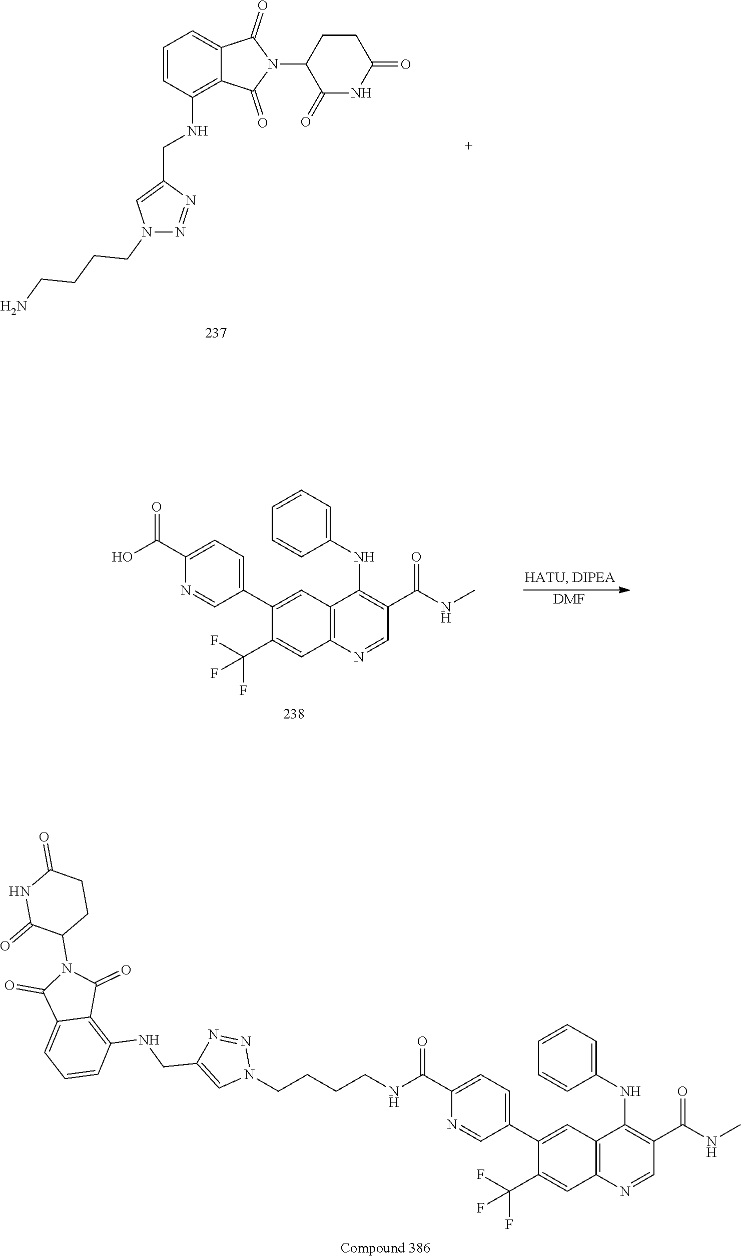

Into a 25 mL single-necked round-bottomed flask containing a well-stirred solution of 4-[[1-(4-aminobutyl)triazol-4-yl]methylamino]-2-(2,6-dioxo-3-piperidyl)isoindoline-1,3-dione (237, 54.73 mg, 128.64 μmol) and 5-[4-anilino-3-(methylcarbamoyl)-7-(trifluoromethyl)-6-quinolyl]pyridine-2-carboxylic acid (238, 50 mg, 107.20 μmol) in DMF (5 mL) were added DIPEA (41.57 mg, 321.60 μmol, 56.02 uL) and HATU (61.14 mg, 160.80 μmol) under nitrogen atmosphere at room temperature. The resulting mixture was stirred at room temperature for 16 h. After completion of reaction 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 6-(6-((4-(4-(((2-(2,6-dioxopiperidin-3-yl)-1,3-dioxoisoindolin-4-yl)amino)methyl)-1H-1,2,3-triazol-1-yl)butyl)carbamoyl)pyridin-3-yl)-N-methyl-4-(phenylamino)-7-(trifluoromethyl)quinoline-3-carboxamide (Compound 386, 25 mg, 28.61 μmol, 26.69% yield). ¹H NMR (400 MHz, DMSO-d₆) δ 11.10 (s, 1H), 10.68 (s, 1H), 8.98 (t, J=6.1 Hz, 1H), 8.94 (s, 1H), 8.61 (d, J=2.1 Hz, 1H), 8.53-8.47 (m, 1H), 8.45 (s, 1H), 8.14 (d, J=8.1 Hz, 1H), 8.04-7.97 (m, 2H), 7.55 (dd, J=8.5, 7.1 Hz, 1H), 7.34 (t, J=7.6 Hz, 2H), 7.22-7.11 (m, 3H), 7.10-7.05 (m, 1H), 7.02 (d, J=7.1 Hz, 1H), 5.05 (dd, J=12.9, 5.3 Hz, 1H), 4.59 (d, J=4.6 Hz, 2H), 4.36 (t, J=6.9 Hz, 2H), 3.32 (p, J=6.4 Hz, 2H), 2.93-2.81 (m, 1H), 2.62-2.53 (m, 1H), 2.36 (d, J=4.5 Hz, 3H), 2.07-1.97 (m, 1H), 1.82 (p, J=7.4 Hz, 2H), 1.55-1.44 (m, 2H). LCMS (ES+): m/z 874 [M+H]⁺

### Synthesis of Compound 387

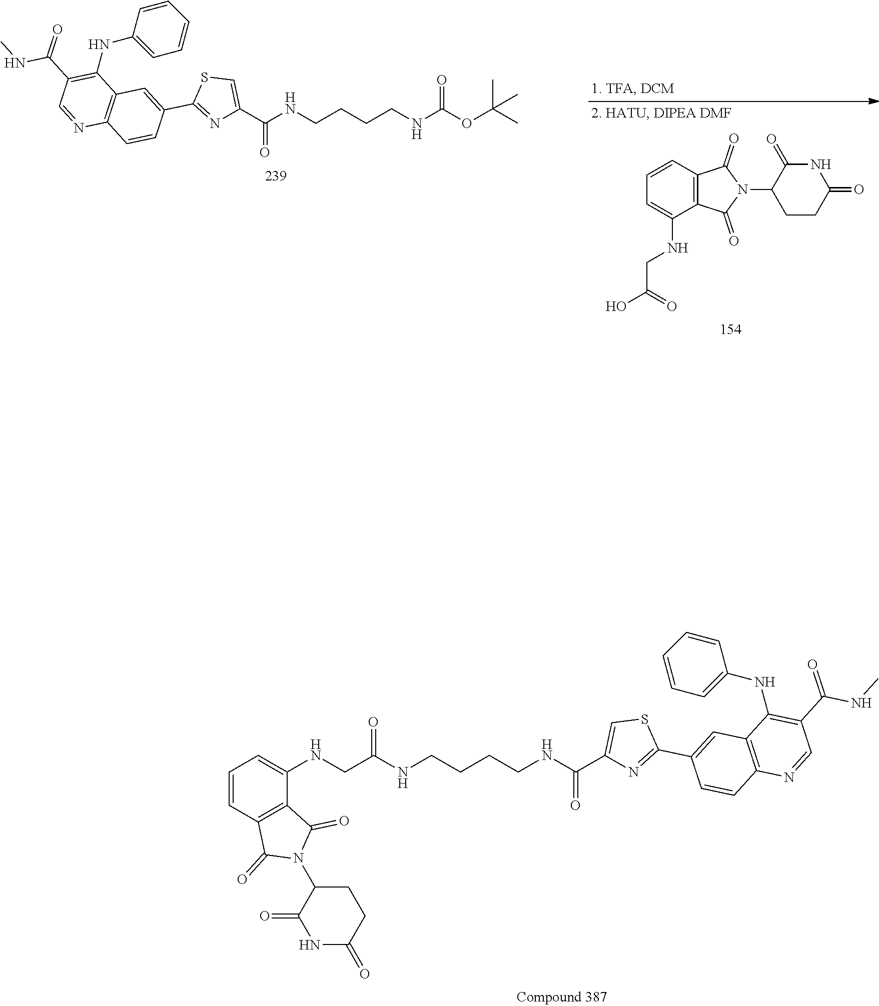 [SMILES: CNC(=O)c1cnc2ccc(-c3nc(C(=O)NCCCCNC(=O)OC(C)(C)C)cs3)cc2c1Nc1ccccc1 ; O=C(O)CNc1cccc2c1C(=O)N(C1CCC(=O)NC1=O)C2=O ; CNC(=O)c1cnc2ccc(-c3nc(C(=O)NCCCCNC(=O)CNc4cccc5c4C(=O)N(C4CCC(=O)NC4=O)C5=O)cs3)cc2c1Nc1ccccc1]

To a stirred solution of tert-butyl N-[4-[[2-[4-anilino-3-(methylcarbamoyl)-6-quinolyl]thiazole-4-carbonyl]amino]butyl]carbamate (239, 104.09 mg, 181.12 μmol) in anhydrous Dichloromethane (5 mL) was added Trifluoroacetic acid (3.05 g, 26.78 mmol, 2.06 mL) at 0° C. The reaction mixture was stirred at room temperature for 3 hours. The reaction mixture was concentrated completely and this crude was taken as such for the next step. To the stirred solution of above crude in N,N-Dimethylformamide (6 mL) was added 2-[[2-(2,6-dioxo-3-piperidyl)-1,3-dioxo-isoindolin-4-yl]amino]acetic acid (154, 60 mg, 181.12 μmol) followed by HATU (103.30 mg, 271.67 μmol) and N,N-Diisopropylethylamine (117.04 mg, 905.58 μmol, 157.73 uL). The reaction mixture stirred at room temperature for 16 hours. Ice cold water (15 mL) was added and stirred for 10 minutes, and then extracted with ethyl acetate. The combined organics were dried over anhydrous sodium sulfate and then concentrated to yield the crude product which was purified by prep-HPLC(SUNFIRE OBD C18(100×30)MM 5μ) Mobile phase: A: 0.1% TFA in water B: ACN) to yield the product 2-[4-anilino-3-(methylcarbamoyl)-6-quinolyl]-N-[4-[[2-[[2-(2,6-dioxo-3-piperidyl)-1,3-dioxo-isoindolin-4-yl]amino]acetyl]amino]butyl]thiazole-4-carboxamide (Compound 387, 13 mg, 16.34 μmol, 9.02% yield) as light yellow colored solid. LCMS (ES+): m/z 788 [M+H]⁺

### Synthesis of Compound 388

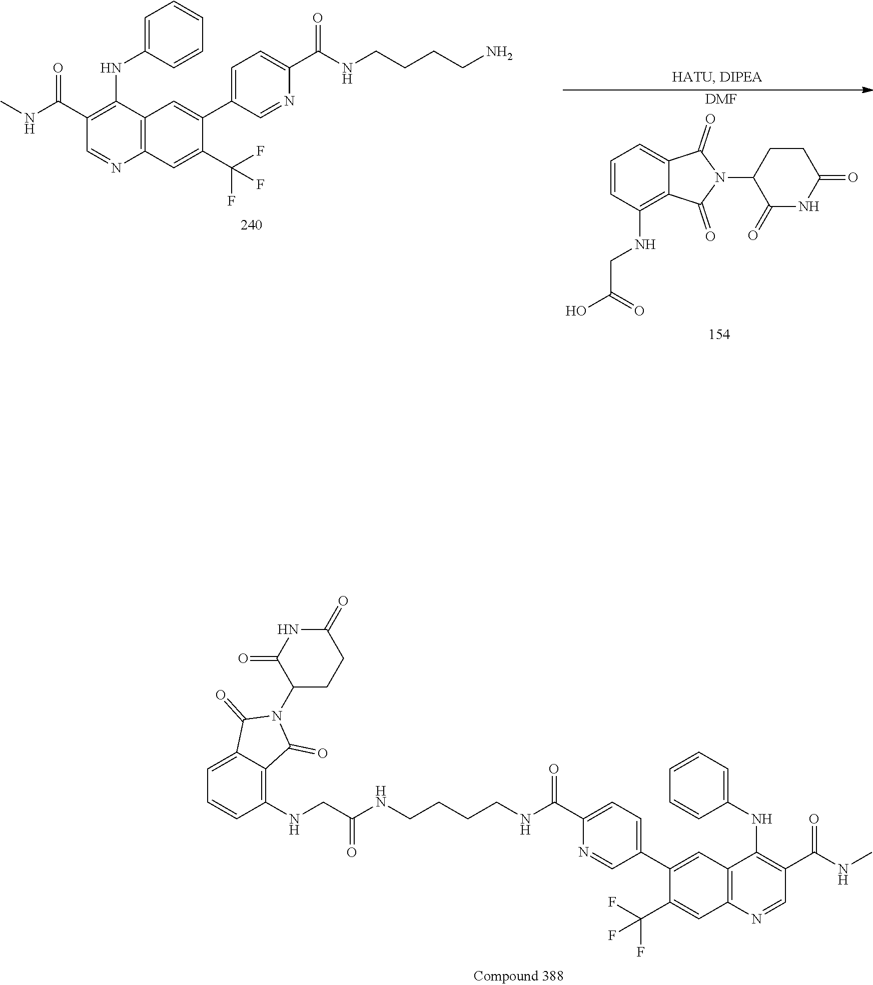

Into a 25 mL single-necked round-bottomed flask containing a well-stirred solution of 2-[[2-(2,6-dioxo-3-piperidyl)-1,3-dioxo-isoindolin-4-yl]amino]acetic acid (154, 50 mg, 150.93 μmol) and 6-[6-(4-aminobutylcarbamoyl)-3-pyridyl]-4-anilino-N-methyl-7-(trifluoromethyl)quinoline-3-carboxamide (240, 80.98 mg, 150.93 μmol) in DMF (10 mL) were added DIPEA (58.52 mg, 452.79 μmol, 78.87 uL) and HATU (86.08 mg, 226.39 μmol) under nitrogen atmosphere at room temperature. The resulting mixture was stirred at room temperature for 16 h. After completion of reaction, 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 6-(6-((4-(2-((2-(2,6-dioxopiperidin-3-yl)-1,3-dioxoisoindolin-4-yl)amino)acetamido)butyl)carbamoyl)pyridin-3-yl)-N-methyl-4-(phenylamino)-7-(trifluoromethyl)quinoline-3-carboxamide (Compound 388, 18 mg, 21.18 μmol, 14.03% yield). ¹H NMR (400 MHz, DMSO-d₆) δ 11.10 (s, 1H), 10.47 (s, 1H), 8.93-8.88 (m, 2H), 8.59 (d, J=2.1 Hz, 1H), 8.49 (q, J=4.3 Hz, 1H), 8.44-8.38 (m, 2H), 8.15-8.08 (m, 2H), 7.99 (dd, J=8.1, 2.2 Hz, 1H), 7.58 (ddd, J=8.6, 7.1, 1.8 Hz, 1H), 7.32 (t, J=7.8 Hz, 2H), 7.19-7.09 (m, 3H), 7.08-7.03 (m, 1H), 6.94 (s, 1H), 6.85 (dd, J=8.6, 3.5 Hz, 1H), 5.07 (dd, J=12.9, 5.4 Hz, 1H), 3.92 (d, J=4.4 Hz, 2H), 3.32 (q, J=6.6 Hz, 2H), 3.16-3.07 (m, 2H), 2.95-2.82 (m, 1H), 2.62-2.55 (m, 2H), 2.40 (d, J=4.4 Hz, 3H), 2.07-1.98 (m, 1H), 1.60-1.50 (m, 2H), 1.50-1.38 (m, 2H). LCMS (ES+): m/z 850 [M+H]⁺

### Synthesis of Compound 389

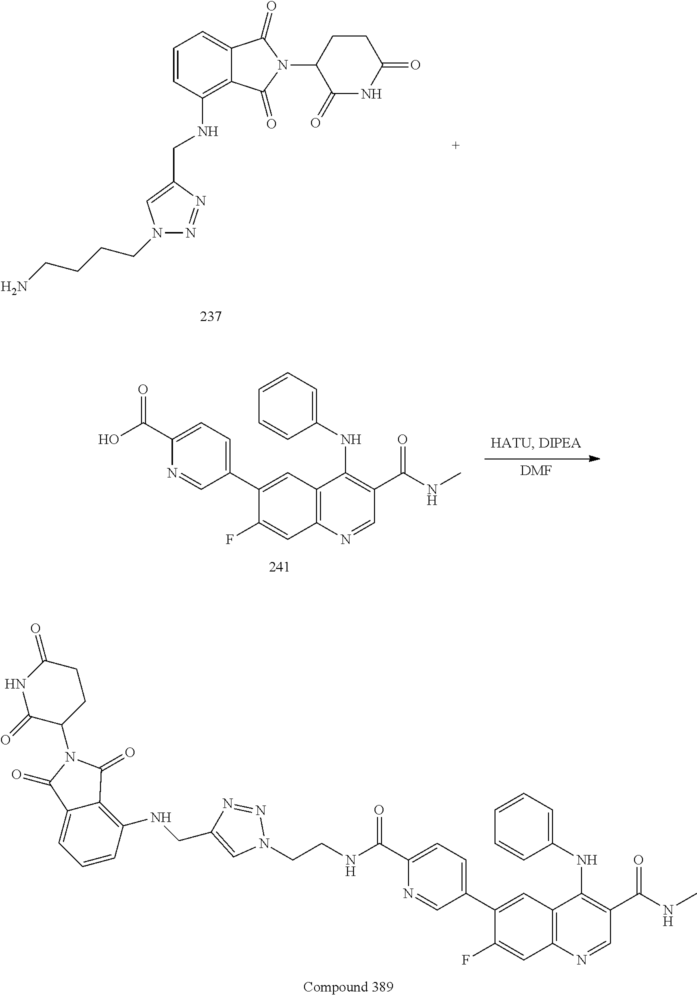

Into a 25 mL single-necked round-bottomed flask containing a well-stirred solution of 4-[[1-(4-aminobutyl)triazol-4-yl]methylamino]-2-(2,6-dioxo-3-piperidyl)isoindoline-1,3-dione (237, 122.60 mg, 288.18 μmol) and 5-[4-anilino-7-fluoro-3-(methylcarbamoyl)-6-quinolyl]pyridine-2-carboxylic acid (241, 100 mg, 240.15 μmol) in DMF (10 mL) were added DIPEA (93.11 mg, 720.45 μmol, 125.49 uL) and HATU (136.97 mg, 360.23 μmol) under nitrogen atmosphere at room temperature. The resulting mixture was stirred at room temperature for 16 h. After completion of reaction, 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 6-(6-((4-(4-(((2-(2,6-dioxopiperidin-3-yl)-1,3-dioxoisoindolin-4-yl)amino)methyl)-1H-1,2,3-triazol-1-yl)butyl)carbamoyl)pyridin-3-yl)-7-fluoro-N-methyl-4-(phenylamino)quinoline-3-carboxamide (Compound 389, 20 mg, 24.28 μmol, 10.11% yield). ¹H NMR (400 MHz, DMSO-d₆) δ 11.09 (s, 1H), 10.21 (s, 1H), 8.92 (t, J=6.2 Hz, 1H), 8.83 (s, 1H), 8.66 (s, 1H), 8.51 (q, J=4.1 Hz, 1H), 8.30 (d, J=8.1 Hz, 1H), 8.10 (s, 2H), 8.03 (s, 1H), 7.85 (d, J=11.7 Hz, 1H), 7.55 (dd, J=8.6, 7.1 Hz, 1H), 7.32 (t, J=7.8 Hz, 2H), 7.17-7.05 (m, 5H), 7.02 (d, J=7.0 Hz, 1H), 5.05 (dd, J=12.9, 5.3 Hz, 1H), 4.59 (d, J=6.1 Hz, 2H), 4.36 (t, J=7.1 Hz, 2H), 2.88 (ddd, J=17.4, 14.0, 5.3 Hz, 1H), 2.63-2.54 (m, 2H), 2.06-1.97 (m, 1H), 1.81 (p, J=7.2 Hz, 2H), 1.49 (p, J=6.8 Hz, 3H). LCMS (ES+): m/z 824 [M+H]⁺

### Synthesis of Compound 390

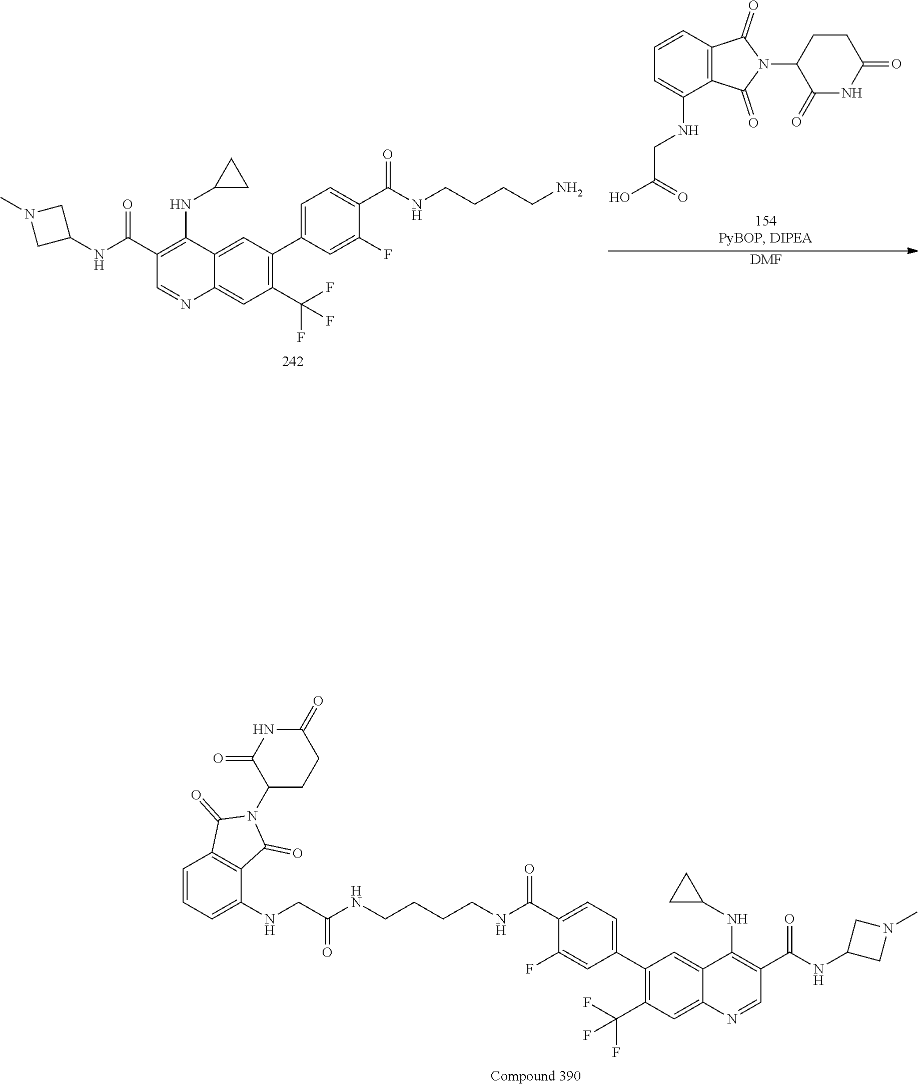

To a stirred solution of 6-[4-(4-aminobutylcarbamoyl)-3-fluoro-phenyl]-4-(cyclopropylamino)-N-(1-methylazetidin-3-yl)-7-(trifluoromethyl)quinoline-3-carboxamide (242, 70 mg, 122.25 μmol) and 2-[[2-(2,6-dioxo-3-piperidyl)-1,3-dioxo-isoindolin-4-yl]amino]acetic acid (154, 48.60 mg, 146.70 μmol) in N,N-Dimethylformamide (4 mL) was added N,N-Diisopropylethylamine (47.40 mg, 366.75 μmol, 63.88 uL) followed by PyBOP (95.43 mg, 183.38 μmol). Upon completion of the reaction, water was added to the reaction mixture and then extracted with ethyl acetate. The combined organics were dried over anhydrous sodium sulfate and then concentrated to yield the crude, which was purified by prep HPLC to yield 4-(cyclopropylamino)-6-(4-((4-(2-((2-(2,6-dioxopiperidin-3-yl)-1,3-dioxoisoindolin-4-yl)amino)acetamido)butyl)carbamoyl)-3-fluorophenyl)-N-(1-methylazetidin-3-yl)-7-(trifluoromethyl)quinoline-3-carboxamide (Compound 390, 6.0 mg, 6.38 μmol, 5.22% yield) as light yellow colored gummy solid. ¹H NMR (400 MHz, Methanol-d₄) δ 8.92 (s, 1H), 8.84 (s, 1H), 8.31 (s, 1H), 7.79 (t, J=7.7 Hz, 1H), 7.57 (dd, J=8.5, 7.2 Hz, 1H), 7.34 (t, J=10.4 Hz, 2H), 7.07 (d, J=7.1 Hz, 1H), 6.89 (d, J=8.5 Hz, 1H), 5.06 (dd, J=12.6, 5.5 Hz, 1H), 4.72-4.56 (m, 2H), 4.44-4.25 (m, 2H), 4.01 (s, 2H), 3.45-3.35 (m, 3H), 3.24 (s, 1H), 3.12-2.99 (m, 3H), 2.92-2.62 (m, 3H), 2.15-2.02 (m, 1H), 1.71-1.55 (m, 4H), 1.30 (d, J=3.7 Hz, 1H), 0.97 (s, 2H), 0.84 (s, 2H). LCMS (ES+): m/z 866 [M+H]⁺

### Synthesis of Compound 391

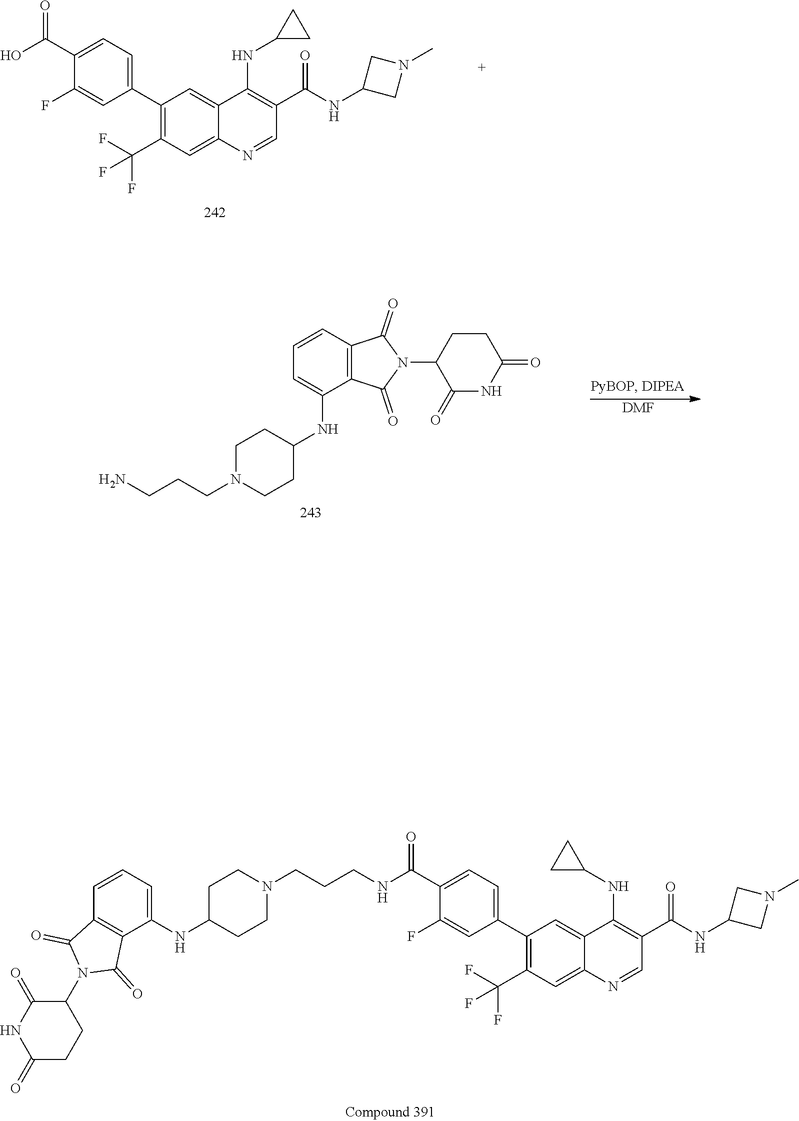

To a stirred solution of 4-[4-(cyclopropylamino)-3-[(1-methylazetidin-3-yl)carbamoyl]-7-(trifluoromethyl)-6-quinolyl]-2-fluoro-benzoic acid (242, 30 mg, 59.71 μmol) and 4-[[1-(3-aminopropyl)-4-piperidyl]amino]-2-(2,6-dioxo-3-piperidyl)isoindoline-1,3-dione (243, 32.09 mg, 77.62 μmol) in N,N-Dimethylformamide (3 mL) was added N,N-Diisopropylethylamine (23.15 mg, 179.12 μmol, 31.20 uL) followed by PyBOP (46.61 mg, 89.56 μmol). The reaction mixture was stirred at room temperature for 16 hours. Water was added to the reaction mixture and then extracted with ethyl acetate. The combined organics were dried over anhydrous sodium sulfate and then concentrated to yield the crude, which was purified by prep HPLC to yield the product 4-(cyclopropylamino)-6-(4-((3-(4-((2-(2,6-dioxopiperidin-3-yl)-1,3-dioxoisoindolin-4-yl)amino)piperidin-1-yl)propyl)carbamoyl)-3-fluorophenyl)-N-(1-methylazetidin-3-yl)-7-(trifluoromethyl)quinoline-3-carboxamide (Compound 391, 1.6 mg, 1.64 μmol, 2.75% yield) as light yellow colored gummy solid. LCMS (ES+): m/z 898 [M+H]⁺

### Synthesis of Compound 392

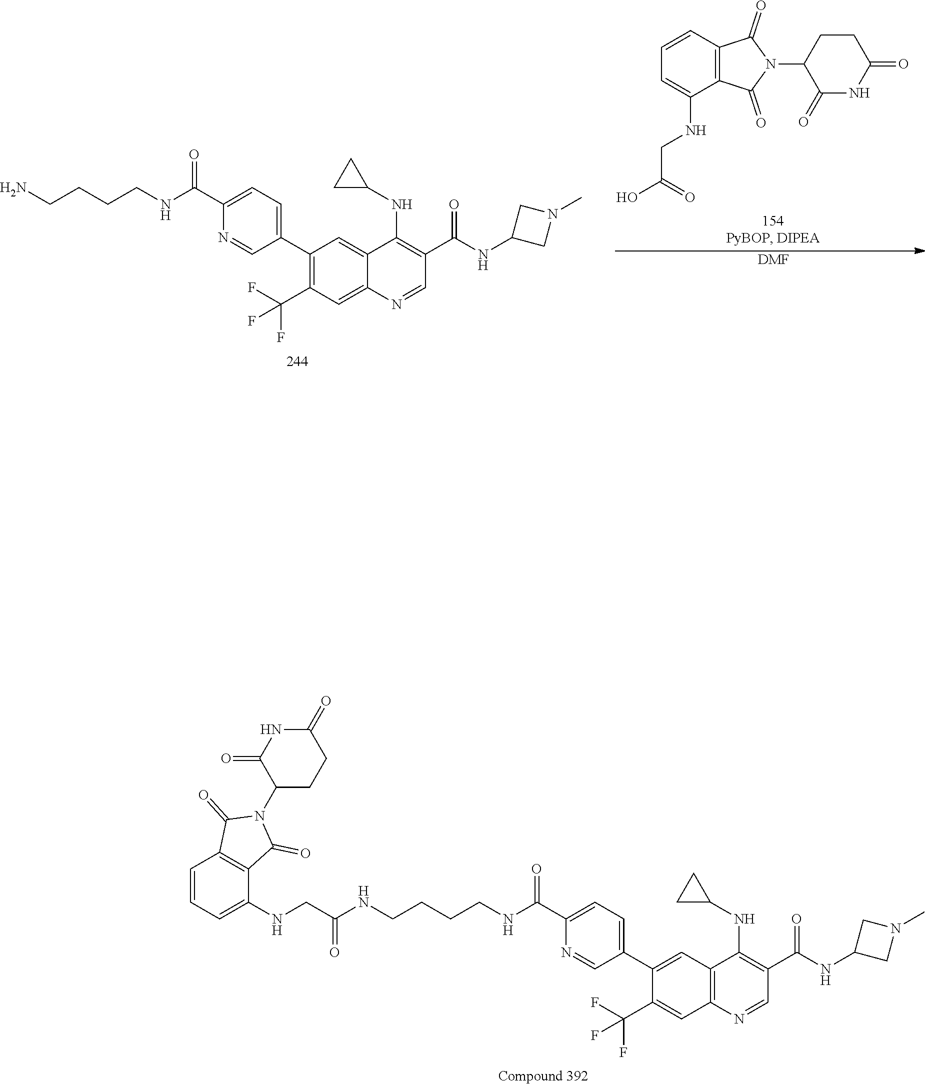

To a stirred solution of 6-[6-(4-aminobutylcarbamoyl)-3-pyridyl]-4-(cyclopropylamino)-N-(1-methylazetidin-3-yl)-7-(trifluoromethyl)quinoline-3-carboxamide (244, 30 mg, 54.00 μmol) and 2-[[2-(2,6-dioxo-3-piperidyl)-1,3-dioxo-isoindolin-4-yl]amino]acetic acid (154, 23.25 mg, 70.20 μmol) in N,N-Dimethylformamide (3 mL) was added N,N-Diisopropylethylamine (34.89 mg, 270.00 μmol, 47.03 uL) followed by PyBOP (42.15 mg, 81.00 μmol). The reaction mixture was stirred at room temperature for 16 hours. Water was added to the reaction mixture and then extracted with ethyl acetate. The combined organics were dried over anhydrous sodium sulfate and then concentrated to yield the crude, which was purified by prep HPLC to yield the product 4-(cyclopropylamino)-6-(6-((4-(2-((2-(2,6-dioxopiperidin-3-yl)-1,3-dioxoisoindolin-4-yl)amino)acetamido)butyl)carbamoyl)pyridin-3-yl)-N-(1-methylazetidin-3-yl)-7-(trifluoromethyl)quinoline-3-carboxamide (Compound 392, 2.0 mg, 2.24 μmol, 4.15% yield) as light yellow colored gummy solid. LCMS (ES+): m/z 869 [M+H]⁺

### Synthesis of Compound 393

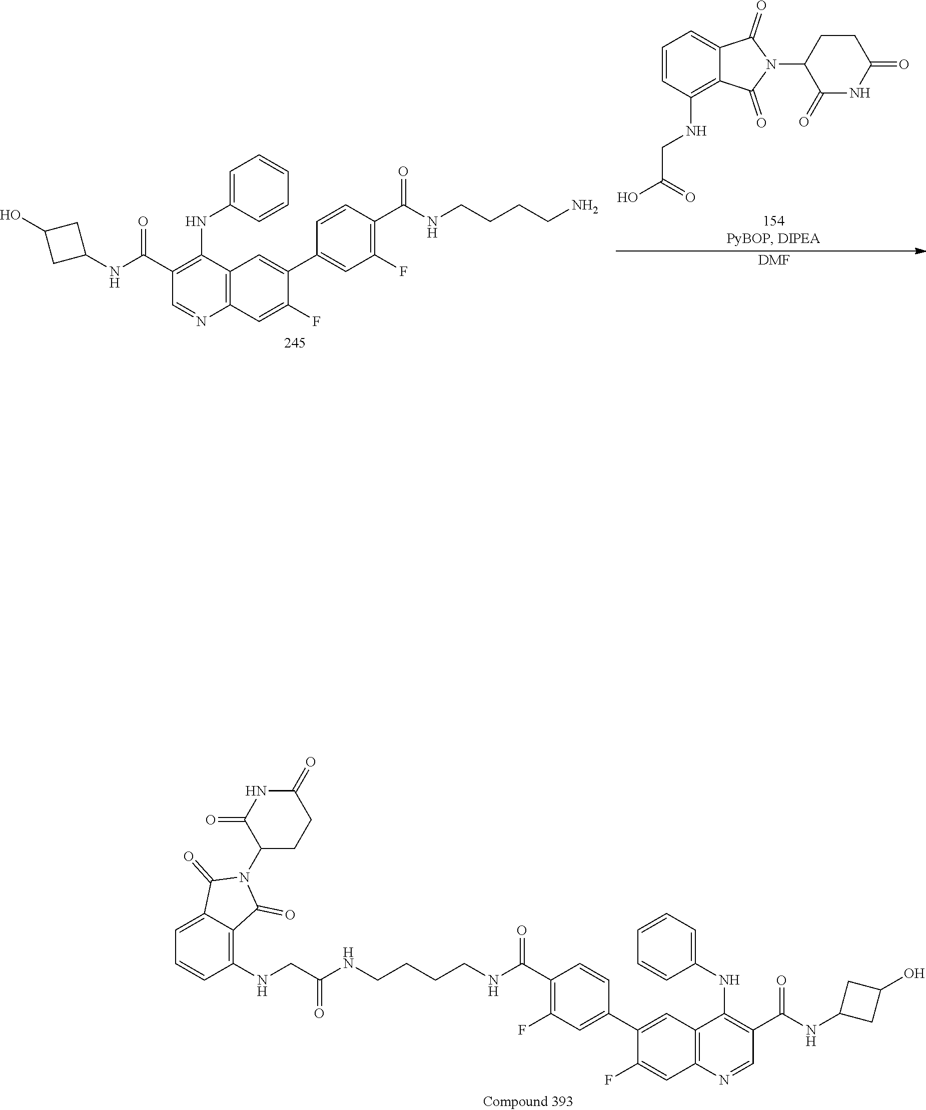

To a stirred solution of 6-[4-(4-aminobutylcarbamoyl)-3-fluoro-phenyl]-4-anilino-7-fluoro-N-(3-hydroxycyclobutyl)quinoline-3-carboxamide (245, 100 mg, 178.70 μmol) and 2-[[2-(2,6-dioxo-3-piperidyl)-1,3-dioxo-isoindolin-4-yl]amino]acetic acid (154, 88.80 mg, 268.05 μmol) in DMF (5 mL) was added PyBOP (139.49 mg, 268.05 μmol) and DIPEA (230.95 mg, 1.79 mmol, 311.25 uL). The reaction mixture was stirred for 16 hr at 25° C. The solvent was evaporated completely under reduced pressure, and the resulting crude was purified by reverse phase preparative HPLC to 6-(4-((4-(2-((2-(2,6-dioxopiperidin-3-yl)-1,3-dioxoisoindolin-4-yl)amino)acetamido)butyl)carbamoyl)-3-fluorophenyl)-7-fluoro-N-(3-hydroxycyclobutyl)-4-(phenylamino)quinoline-3-carboxamide (Compound 393, 14 mg, 15.65 μmol, 8.76% yield) as a light yellow color solid. ¹H NMR (400 MHz, DMSO-d₆) δ 11.11 (s, 1H), 10.05 (s, 1H), 8.83 (s, 1H), 8.67 (d, J=7.2 Hz, 1H), 8.41-8.35 (m, 1H), 8.22-8.16 (m, 2H), 8.13 (t, J=5.6 Hz, 1H), 7.80 (dd, J=12.1, 2.6 Hz, 1H), 7.64 (t, J=7.8 Hz, 1H), 7.59 (t, J=7.9 Hz, 1H), 7.37-7.27 (m, 4H), 7.10-7.02 (m, 4H), 6.95 (t, J=5.6 Hz, 1H), 6.86 (d, J=8.6 Hz, 1H), 5.07 (dd, J=12.9, 5.4 Hz, 1H), 3.93 (d, J=5.7 Hz, 2H), 3.76 (p, J=7.5, 6.9 Hz, 1H), 3.61 (q, J=8.1 Hz, 1H), 3.27-3.22 (m, 3H), 3.18-3.09 (m, 3H), 2.94-2.83 (m, 1H), 2.62-2.56 (m, 2H), 2.42-2.35 (m, 2H), 2.08-1.98 (m, 1H), 1.77 (dt, J=11.4, 8.2 Hz, 2H), 1.56-1.42 (m, 4H). LCMS (ES+): m/z 873 [M+H]⁺

### Synthesis of Compound 394

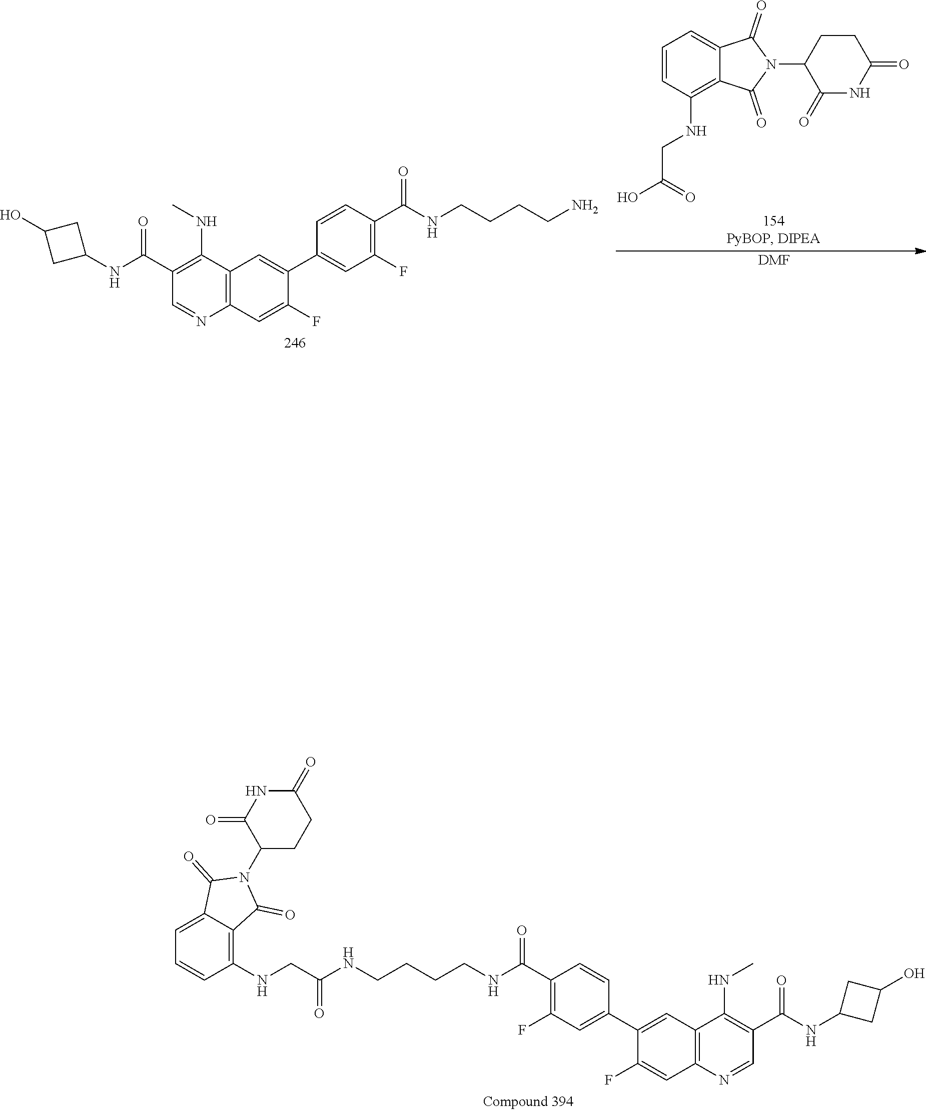

To a stirred solution of 6-[4-(4-aminobutylcarbamoyl)-3-fluoro-phenyl]-7-fluoro-N-(3-hydroxycyclobutyl)-4-(methylamino)quinoline-3-carboxamide (246, 70 mg, 140.69 μmol) and 2-[[2-(2,6-dioxo-3-piperidyl)-1,3-dioxo-isoindolin-4-yl]amino]acetic acid (154, 69.91 mg, 211.04 μmol) in DMF (5 mL) was added PyBOP (109.82 mg, 211.04 μmol) and DIPEA (181.83 mg, 1.41 mmol, 245.06 uL). The reaction mixture was stirred for 16 hr at 25° C. The solvent was evaporated completely under reduced pressure, and the resulting crude was purified by reverse phase preparative HPLC to yield 6-(4-((4-(2-((2-(2,6-dioxopiperidin-3-yl)-1,3-dioxoisoindolin-4-yl)amino)acetamido)butyl)carbamoyl)-3-fluorophenyl)-7-fluoro-N-(3-hydroxycyclobutyl)-4-(methylamino)quinoline-3-carboxamide (Compound 394, 9 mg, 10.99 μmol, 7.81% yield) as a light yellow color solid. LCMS (ES+): m/z 811 [M+H]⁺

### Synthesis of Compound 395

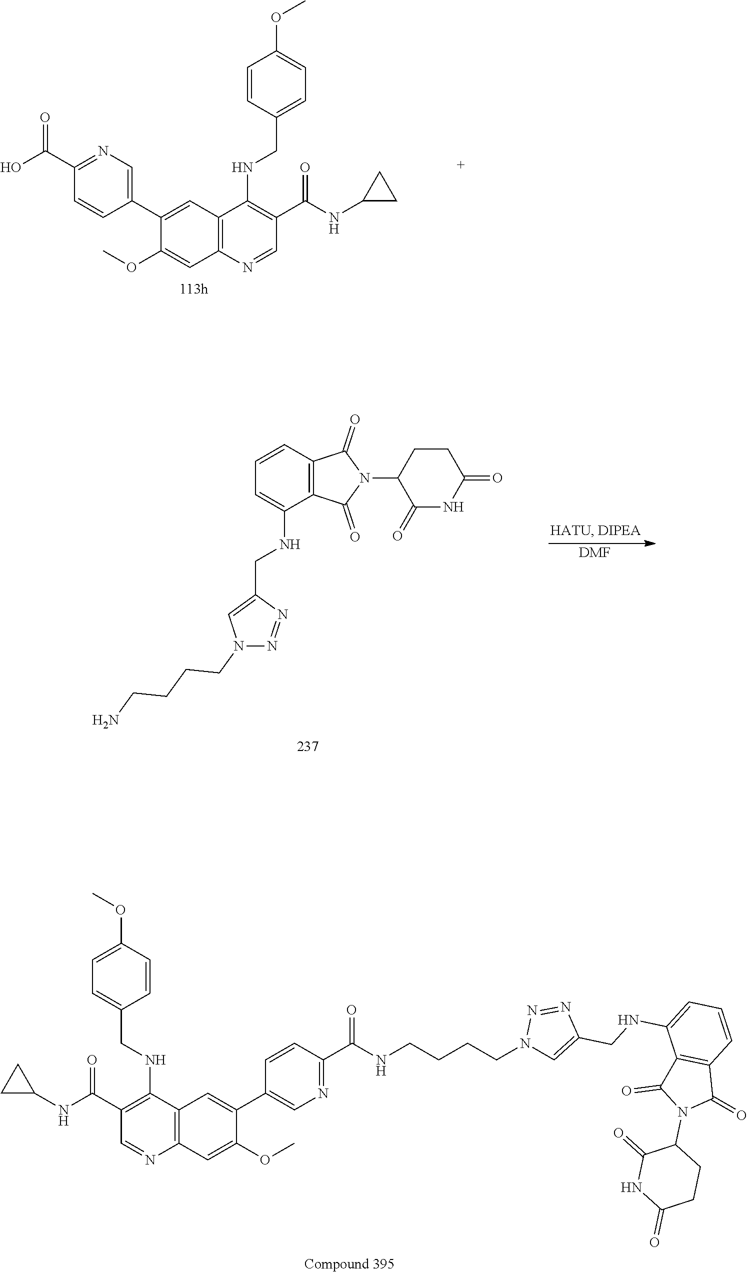

To a stirred solution of 5-[3-(cyclopropylcarbamoyl)-7-methoxy-4-[(4-methoxyphenyl)methylamino]-6-quinolyl]pyridine-2-carboxylic acid (113 h, 70 mg, 140.41 μmol) and 4-[[1-(4-aminobutyl)triazol-4-yl]methylamino]-2-(2,6-dioxo-3-piperidyl)isoindoline-1,3-dione (237, 59.74 mg, 140.41 μmol) in DMF (7 mL) was added DIPEA (90.74 mg, 702.07 μmol, 122.29 uL) and HATU (80.08 mg, 210.62 μmol). The resulting mixture was stirred for 16 hr at 25° C. The mixture was diluted with water (20 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[6-[4-[4-[[[2-(2,6-dioxo-3-piperidyl)-1,3-dioxo-isoindolin-4-yl]amino]methyl]triazol-1-yl]butylcarbamoyl]-3-pyridyl]-7-methoxy-4-[(4-methoxyphenyl)methylamino]quinoline-3-carboxamide (Compound 395, 120 mg, 127.41 μmol, 90.74% yield) as an yellow solid. ¹H NMR (400 MHz, Methanol-d₄) δ 8.73 (s, 1H), 8.50 (s, 1H), 8.37 (s, 1H), 8.08 (d, J=8.2 Hz, 1H), 8.04-8.00 (m, 1H), 7.92 (s, 1H), 7.46 (dd, J=8.6, 7.1 Hz, 1H), 7.33 (s, 1H), 7.28-7.23 (m, 2H), 7.03 (d, J=8.5 Hz, 1H), 6.96 (d, J=7.1 Hz, 1H), 6.90-6.86 (m, 2H), 5.00 (dd, J=12.5, 5.3 Hz, 1H), 4.62 (s, 2H), 4.45 (t, J=6.9 Hz, 2H), 4.01 (s, 3H), 3.75 (d, J=1.6 Hz, 3H), 3.44 (t, J=6.8 Hz, 2H), 2.88-2.78 (m, 2H), 2.77-2.59 (m, 3H), 2.10-2.01 (m, 1H), 2.00-1.91 (m, 3H), 1.66-1.52 (m, 2H), 0.83-0.75 (m, 2H), 0.59 (q, J=5.0, 4.0 Hz, 2H). LCMS (ES+): m/z 906 [M+H]⁺

### Synthesis of Compound 396

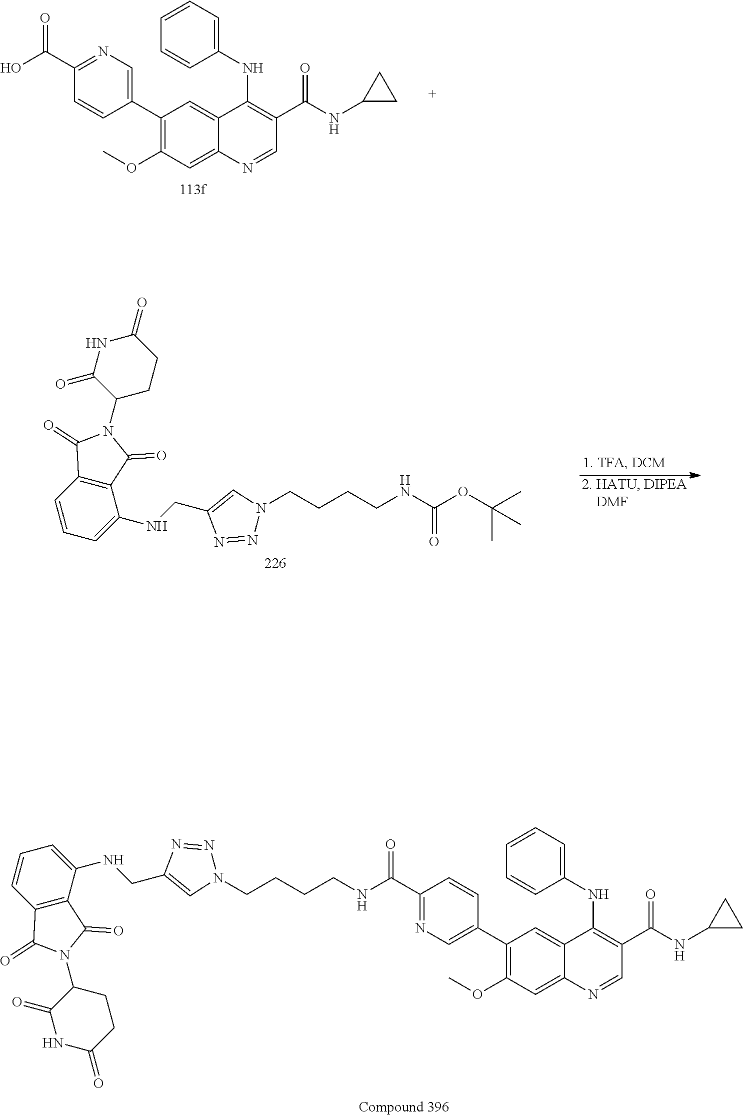 [SMILES: COc1cc2ncc(C(=O)NC3CC3)c(Nc3ccccc3)c2cc1-c1ccc(C(=O)O)nc1 ; CC(C)(C)OC(=O)NCCCCn1cc(CNc2cccc3c2C(=O)N(C2CCC(=O)NC2=O)C3=O)nn1 ; "2. HATU, DIPEA    DMF" ; COc1cc2ncc(C(=O)NC3CC3)c(Nc3ccccc3)c2cc1-c1ccc(C(=O)NCCCCn2cc(CNc3cccc4c3C(=O)N(C3CCC(=O)NC3=O)C4=O)nn2)nc1]

Into a 25 mL single-necked round-bottomed flask containing a well-stirred solution of tert-butyl N-[4-[4-[[[2-(2,6-dioxo-3-piperidyl)-1,3-dioxo-isoindolin-4-yl]amino]methyl]triazol-1-yl]butyl]carbamate (226, 101.76 mg, 193.63 μmol) in anhydrous DCM (3 mL) was added TFA (100.35 mg, 880.13 μmol, 67.81 uL) under nitrogen atmosphere at room temperature. The resulting mixture was stirred at room temperature for 30 minutes. The reaction mixture was concentrated under reduced pressure to afford a crude residue which was dissolved in DMF (2 mL). DIPEA (68.25 mg, 528.08 μmol, 91.98 uL), 5-[4-anilino-3-(cyclopropylcarbamoyl)-7-methoxy-6-quinolyl]pyridine-2-carboxylic acid (113f, 80 mg, 176.03 μmol) and HATU (100.40 mg, 264.04 μmol) were added at room temperature. The resulting mixture was stirred at room temperature for 16 h. The reaction mixture was concentrated to afford crude residue which was purified by prep-HPLC (SUNFIRE OBD C18(100×30)MM 5μ) Mobile phase: A: 0.1% TFA in water B: ACN to afford 4-anilino-N-cyclopropyl-6-[6-[4-[4-[[[2-(2,6-dioxo-3-piperidyl)-1,3-dioxo-isoindolin-4-yl]amino]methyl]triazol-1-yl]butylcarbamoyl]-3-pyridyl]-7-methoxy-quinoline-3-carboxamide (Compound 396, 13 mg, 14.00 μmol, 7.95% yield) as a yellow solid. ¹H NMR (400 MHz, DMSO-d₆) δ 11.08 (s, 1H), 8.88 (t, J=6.1 Hz, 1H), 8.77 (s, 1H), 8.70 (s, 1H), 8.58 (d, J=3.4 Hz, 1H), 8.43 (s, 1H), 8.12-8.04 (m, 2H), 8.02 (s, 1H), 7.55 (t, J=7.8 Hz, 1H), 7.49 (s, 1H), 7.42 (t, J=7.7 Hz, 2H), 7.30 (t, J=7.3 Hz, 1H), 7.23 (d, J=7.7 Hz, 2H), 7.15 (d, J=8.6 Hz, 1H), 7.10-7.04 (m, 1H), 7.02 (d, J=7.1 Hz, 1H), 5.05 (dd, J=12.9, 5.4 Hz, 1H), 4.59 (d, J=5.8 Hz, 2H), 4.36 (t, J=7.1 Hz, 2H), 4.01 (s, 3H), 3.35-3.29 (m, 2H), 2.95-2.82 (m, 1H), 2.63 (d, J=28.3 Hz, 3H), 2.33 (d, J=2.7 Hz, 2H), 2.07-1.96 (m, 1H), 1.86-1.74 (m, 2H), 1.50 (q, J=7.3 Hz, 2H), 0.58-0.49 (m, 2H), 0.31-0.21 (m, 2H). LCMS (ES+): m/z 863 [M+]⁺

### Synthesis of Compound 397

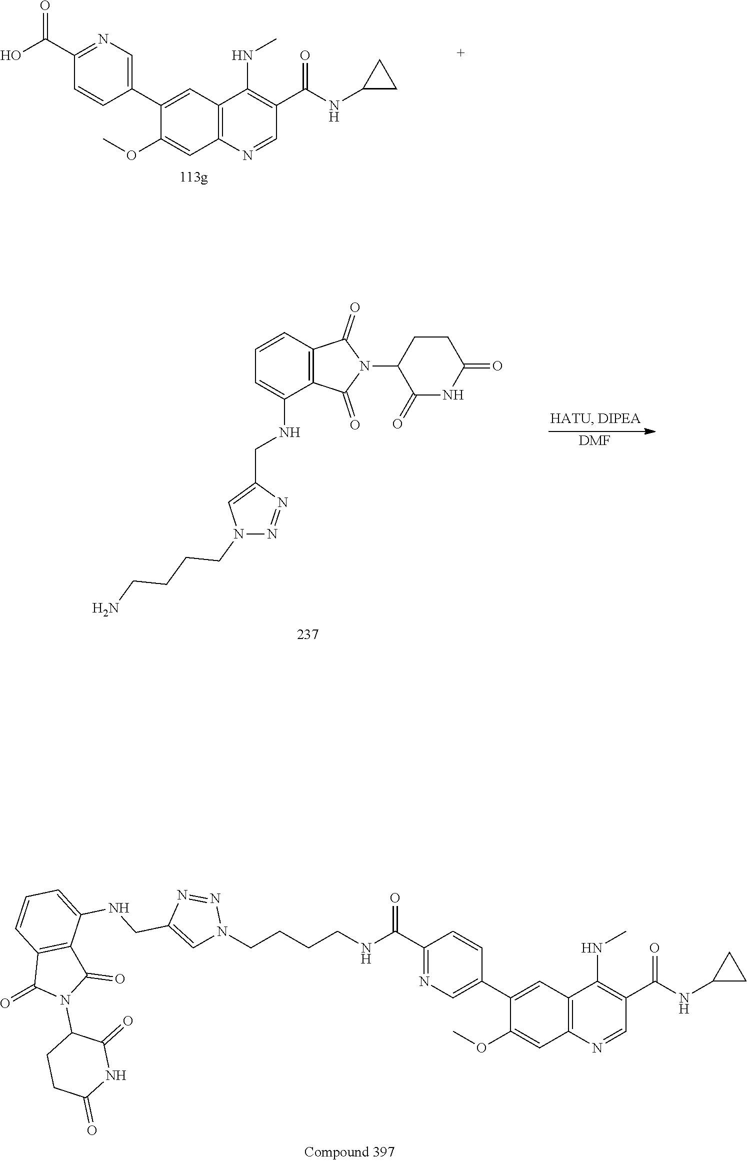

To a stirred solution of 5-[3-(cyclopropylcarbamoyl)-7-methoxy-4-(methylamino)-6-quinolyl]pyridine-2-carboxylic acid (113 g, 98 mg, 249.74 μmol) and 4-[[1-(4-aminobutyl)triazol-4-yl]methylamino]-2-(2,6-dioxo-3-piperidyl)isoindoline-1,3-dione (237, 127.50 mg, 299.69 μmol) in DMF (10 mL) was added DIPEA (161.39 mg, 1.25 mmol, 217.50 uL) and HATU (142.44 mg, 374.61 μmol). The resulting mixture was stirred for 16 hr at 25° C. The resulting mixture was diluted with water (20 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[6-[4-[4-[[[2-(2,6-dioxo-3-piperidyl)-1,3-dioxo-isoindolin-4-yl]amino]methyl]triazol-1-yl]butylcarbamoyl]-3-pyridyl]-7-methoxy-4-(methylamino)quinoline-3-carboxamide (Compound 397, 30 mg, 36.66 μmol, 14.68% yield) as an yellow solid. ¹H NMR (400 MHz, DMSO-d₆) δ 13.83 (s, 1H), 11.08 (s, 1H), 9.25 (s, 1H), 8.90 (t, J=6.1 Hz, 1H), 8.86-8.82 (m, 2H), 8.58 (s, 1H), 8.55 (s, 1H), 8.22 (dd, J=8.1, 2.2 Hz, 1H), 8.15 (d, J=8.1 Hz, 1H), 8.03 (s, 1H), 7.56 (dd, J=8.6, 7.1 Hz, 1H), 7.42 (s, 1H), 7.15 (d, J=8.6 Hz, 1H), 7.10-7.04 (m, 1H), 7.03 (d, J=7.0 Hz, 1H), 5.05 (dd, J=12.9, 5.4 Hz, 1H), 4.63-4.55 (m, 2H), 4.37 (t, J=7.1 Hz, 2H), 3.98 (s, 3H), 3.34 (q, J=6.7 Hz, 2H), 3.07 (s, 2H), 2.85 (ddp, J=11.2, 7.9, 3.6 Hz, 2H), 2.64-2.52 (m, 1H), 2.06-1.97 (m, 1H), 1.83 (p, J=7.3 Hz, 2H), 1.51 (p, J=7.0 Hz, 2H), 0.75 (td, J=7.1, 4.8 Hz, 2H), 0.64-0.56 (m, 2H). LCMS (ES+): m/z 800 [M+H]⁺

### Synthesis of Compound 398

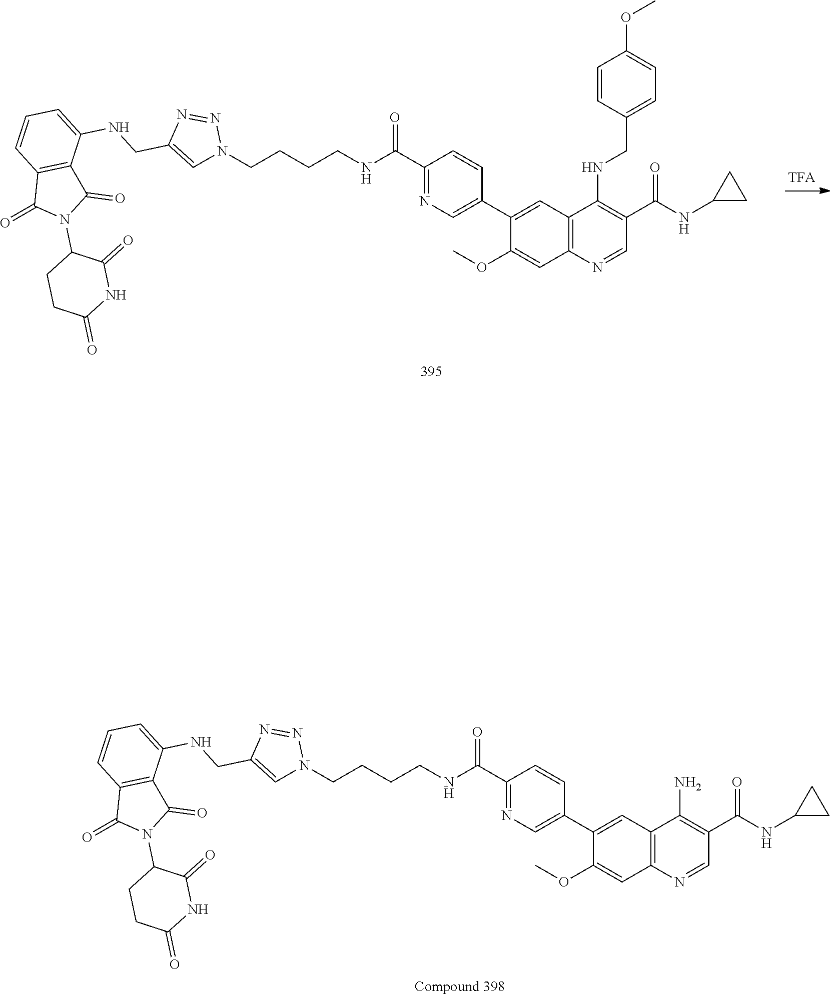 [SMILES: COc1ccc(CNc2c(C(=O)NC3CC3)cnc3cc(OC)c(-c4ccc(C(=O)NCCCCn5cc(CNc6cccc7c6C(=O)N(C6CCC(=O)NC6=O)C7=O)nn5)nc4)cc23)cc1 ; COc1cc2ncc(C(=O)NC3CC3)c(N)c2cc1-c1ccc(C(=O)NCCCCn2cc(CNc3cccc4c3C(=O)N(C3CCC(=O)NC3=O)C4=O)nn2)nc1]

To a stirred solution of N-cyclopropyl-6-[6-[4-[4-[[[2-(2,6-dioxo-3-piperidyl)-1,3-dioxo-isoindolin-4-yl]amino]methyl]triazol-1-yl]butylcarbamoyl]-3-pyridyl]-7-methoxy-4-[(4-methoxyphenyl)methylamino]quinoline-3-carboxamide (395, 100 mg, 110.38 μmol) in Trifluoroacetic acid (2.96 g, 25.96 mmol, 2 mL) was heated to 80° C. and stirred for 3 hr at 80° C. The resulting mixture was concentrated completely under reduced pressure and the crude product was purified by prep HPLC to yield 4-amino-N-cyclopropyl-6-[6-[4-[4-[[[2-(2,6-dioxo-3-piperidyl)-1,3-dioxo-isoindolin-4-yl]amino]methyl]triazol-1-yl]butylcarbamoyl]-3-pyridyl]-7-methoxy-quinoline-3-carboxamide (Compound 398, 5 mg, 6.25 μmol, 5.66% yield) as a yellow solid. ¹H NMR (400 MHz, DMSO-d₆) δ 14.03 (s, 1H), 11.09 (s, 1H), 9.97 (s, 1H), 9.56 (s, 1H), 8.92 (t, J=6.1 Hz, 1H), 8.87-8.78 (m, 3H), 8.72 (s, 1H), 8.25-8.19 (m, 1H), 8.17-8.12 (m, 1H), 8.03 (d, J=2.1 Hz, 1H), 7.59-7.52 (m, 1H), 7.43 (d, J=2.1 Hz, 1H), 7.17-7.12 (m, 1H), 7.07 (t, J=6.0 Hz, 1H), 7.02 (dd, J=7.0, 2.0 Hz, 1H), 5.05 (dd, J=12.8, 5.1 Hz, 1H), 4.59 (d, J=5.9 Hz, 2H), 4.37 (t, J=6.9 Hz, 2H), 4.00 (d, J=2.0 Hz, 3H), 2.86 (dq, J=12.2, 4.8, 4.2 Hz, 2H), 2.60 (s, 2H), 2.08-1.97 (m, 1H), 1.82 (p, J=7.2 Hz, 2H), 1.56-1.43 (m, 2H), 0.82-0.72 (m, 2H), 0.67-0.58 (m, 2H). LCMS (ES+): m/z 786 [M+H]⁺

### Synthesis of Compound 399

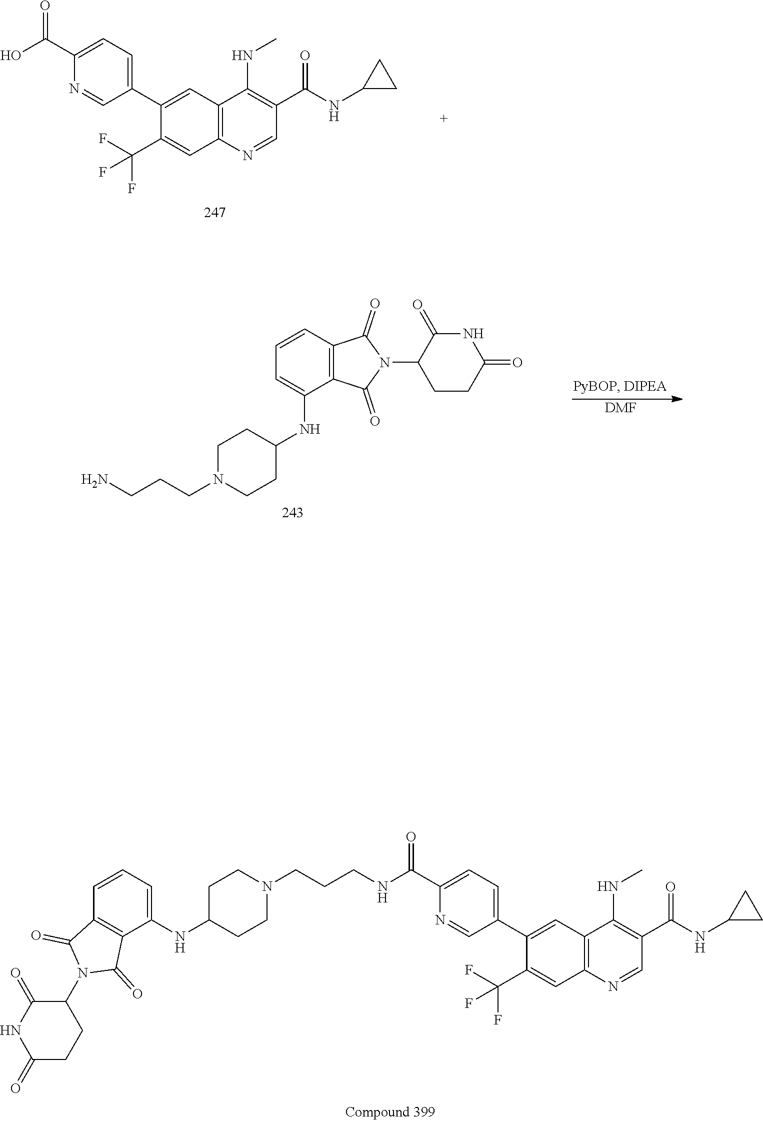

To a solution of 5-[3-(cyclopropylcarbamoyl)-4-(methylamino)-7-(trifluoromethyl)-6-quinolyl]pyridine-2-carboxylic acid (247, 50 mg, 116.18 μmol) and 4-[[1-(3-aminopropyl)-4-piperidyl]amino]-2-(2,6-dioxo-3-piperidyl)isoindoline-1,3-dione (243, 52.84 mg, 127.79 μmol) in DMF (5 mL) was added DIPEA (75.07 mg, 580.88 μmol, 101.18 uL) followed by PyBOP (90.69 mg, 174.27 μmol) and the reaction mixture was stirred at room temperature for 16 hours. Ice cold water (15 mL) was added to the reaction mixture and stirred for 5 minutes, and then extracted with ethyl acetate. The combined organics were dried over anhydrous sodium sulfate and then concentrated to yield the crude product, which was purified by prep-HPLC(SUNFIRE OBD C18(100×30)MM 5μ) Mobile phase: A: 0.1% TFA in water B: ACN) to yield N-cyclopropyl-6-[6-[3-[4-[[2-(2,6-dioxo-3-piperidyl)-1,3-dioxo-isoindolin-4-yl]amino]-1-piperidyl]propylcarbamoyl]-3-pyridyl]-4-(methylamino)-7-(trifluoromethyl)quinoline-3-carboxamide (Compound 399, 20 mg, 24.17 μmol, 20.80% yield) as light yellow colored solid. ¹H NMR (400 MHz, Methanol-d₄) δ 8.76 (s, 1H), 8.73 (s, 1H), 8.54 (s, 1H), 8.33 (s, 1H), 8.30 (d, J=8.2 Hz, 1H), 8.11 (d, J=7.9 Hz, 1H), 7.67-7.60 (m, 1H), 7.20 (d, J=8.6 Hz, 1H), 7.16 (d, J=7.1 Hz, 1H), 5.09 (dd, J=12.4, 5.4 Hz, 1H), 3.94 (s, 1H), 3.75 (d, J=12.4 Hz, 2H), 3.63 (t, J=6.4 Hz, 2H), 3.21 (t, J=12.9 Hz, 2H), 3.00-2.92 (m, 2H), 2.91-2.86 (m, 1H), 2.85 (d, J=5.4 Hz, 1H), 2.80-2.70 (m, 3H), 2.41 (d, J=14.2 Hz, 2H), 2.21-2.10 (m, 4H), 1.93-1.77 (m, 3H), 0.95-0.85 (m, 2H), 0.74-0.66 (m, 2H). LCMS (ES+): m/z 826 [M+H]⁺

### Synthesis of Compound 400

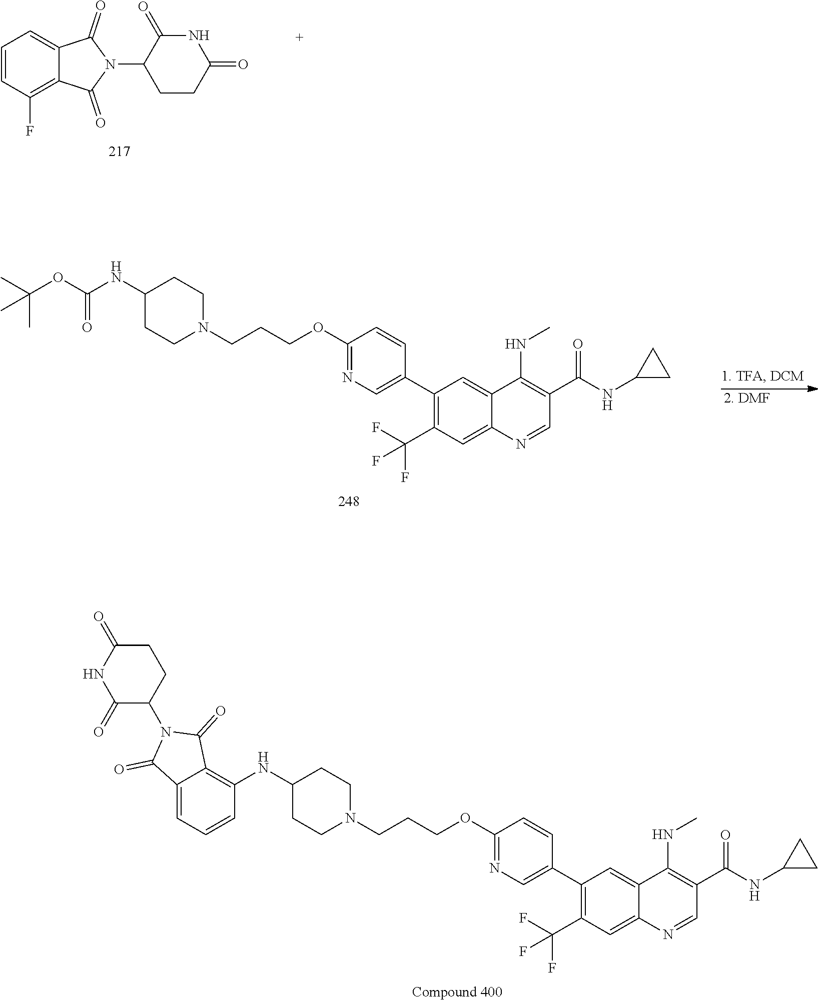 [SMILES: O=C1CCC(N2C(=O)c3cccc(F)c3C2=O)C(=O)N1 ; CNc1c(C(=O)NC2CC2)cnc2cc(C(F)(F)F)c(-c3ccc(OCCCN4CCC(NC(=O)OC(C)(C)C)CC4)nc3)cc12 ; "2. DMF" ; CNc1c(C(=O)NC2CC2)cnc2cc(C(F)(F)F)c(-c3ccc(OCCCN4CCC(Nc5cccc6c5C(=O)N(C5CCC(=O)NC5=O)C6=O)CC4)nc3)cc12]

Into a 25 mL single-necked round-bottomed flask containing a well-stirred solution of tert-butyl N-[1-[3-[[5-[3-(cyclopropylcarbamoyl)-4-(methylamino)-7-(trifluoromethyl)-6-quinolyl]-2-pyridyl]oxy]propyl]-4-piperidyl]carbamate (248, 100 mg, 155.59 μmol) in DCM (5 mL) was added TFA (88.70 mg, 777.96 μmol, 59.94 uL) under nitrogen atmosphere at room temperature. The resulting mixture was stirred at room temperature for 30 minutes. The reaction mixture was concentrated under reduced pressure to afford a residue which was dissolved in DMF (5 mL). DIPEA (20.11 mg, 155.59 μmol, 27.10 uL) and 2-(2,6-dioxo-3-piperidyl)-4-fluoro-isoindoline-1,3-dione (217, 64.47 mg, 233.39 μmol) were added under nitrogen atmosphere at room temperature. The resulting mixture was heated at 100° C. for 16 h. The reaction mixture was concentrated under reduced pressure and purified by prep-HPLC (SUNFIRE OBD C18(100×30)MM 5μ) Mobile phase: A: 0.1% TFA in water B: ACN to afford N-cyclopropyl-6-[6-[3-[4-[[2-(2,6-dioxo-3-piperidyl)-1,3-dioxo-isoindolin-4-yl]amino]-1-piperidyl]propoxy]-3-pyridyl]-4-(methylamino)-7-(trifluoromethyl)quinoline-3-carboxamide (Compound 400, 13 mg, 16.24 μmol, 10.44% yield) as a yellow solid. ¹H NMR (400 MHz, DMSO-d₆) δ 11.11 (s, 1H), 9.33 (s, 1H), 8.86 (s, 1H), 8.71 (s, 1H), 8.55 (s, 1H), 8.31 (s, 1H), 7.89 (d, J=2.6 Hz, 1H), 7.64 (t, J=7.8 Hz, 1H), 7.58-7.52 (m, 1H), 7.23 (d, J=9.0 Hz, 1H), 7.11 (d, J=7.1 Hz, 1H), 6.58 (d, J=9.4 Hz, 1H), 6.30 (d, J=8.1 Hz, 1H), 5.06 (dd, J=12.8, 5.5 Hz, 1H), 4.05 (d, J=7.2 Hz, 2H), 3.84 (s, 2H), 3.58 (s, 2H), 3.29 (s, 2H), 3.16-3.02 (m, 4H), 2.93-2.81 (m, 3H), 2.63-2.57 (m, 1H), 2.20 (d, J=13.1 Hz, 2H), 2.16-2.07 (m, 2H), 2.07-1.98 (m, 2H), 1.80-1.66 (m, 2H), 0.74 (dt, J=6.8, 3.3 Hz, 2H), 0.63-0.56 (m, 2H). LCMS (ES+): m/z 799 [M+H]⁺

### Synthesis of Compound 401

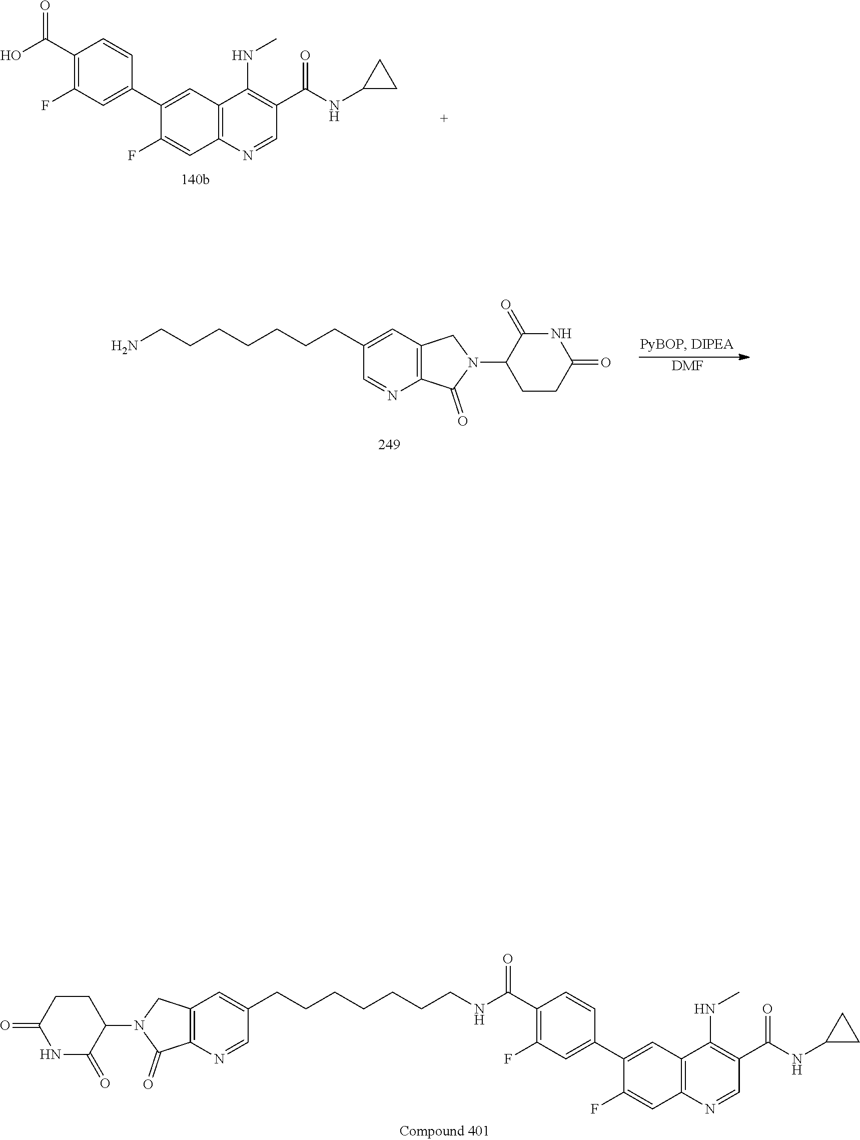

To a solution of 4-[3-(cyclopropylcarbamoyl)-7-fluoro-4-(methylamino)-6-quinolyl]-2-fluoro-benzoic acid (140b, 50 mg, 125.83 μmol) and 3-[3-(7-aminoheptyl)-7-oxo-5H-pyrrolo[3,4-b]pyridin-6-yl]piperidine-2,6-dione (249, 54.12 mg, 150.99 μmol) in DMF (5 mL) was added DIPEA (81.31 mg, 629.13 μmol, 109.58 uL) and PyBOP (98.22 mg, 188.74 μmol). The resulting mixture was stirred for 16 hr at 25° C. Added water (20 mL) to the reaction mixture and extracted with ethyl acetate (3×25 mL). The combined organic extracts were dried over anhydrous sodium sulfate, filtered and evaporated the solvent completely under reduced pressure. The resulting crude was purified by Reverse phase prep HPLC to yield N-cyclopropyl-6-[4-[7-[6-(2,6-dioxo-3-piperidyl)-7-oxo-5H-pyrrolo[3,4-b]pyridin-3-yl]heptylcarbamoyl]-3-fluoro-phenyl]-7-fluoro-4-(methylamino)quinoline-3-carboxamide (Compound 401, 10 mg, 12.82 μmol, 10.19% yield) as an off white solid. LCMS (ES+): m/z 738 [M+H]⁺

### Synthesis of Compound 402

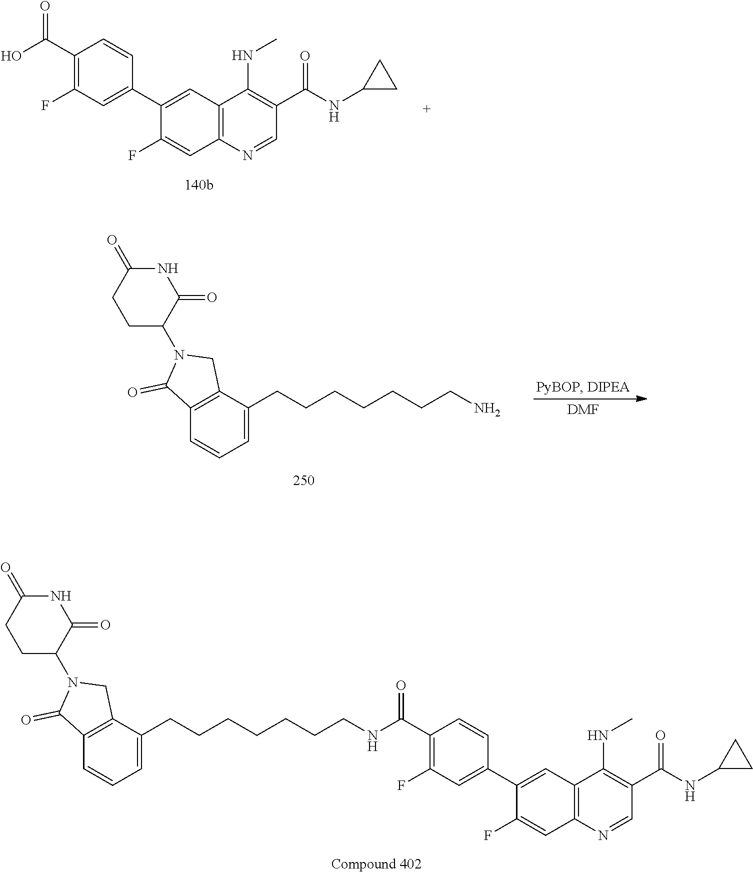

To a stirred solution of 4-[3-(cyclopropylcarbamoyl)-7-fluoro-4-(methylamino)-6-quinolyl]-2-fluoro-benzoic acid (140b, 50 mg, 125.83 μmol) in DMF (5 mL) was added 3-[4-(7-aminoheptyl)-1-oxo-isoindolin-2-yl]piperidine-2,6-dione (250, 44.98 mg, 125.83 μmol), PyBOP (98.22 mg, 188.74 μmol), and DIPEA (162.62 mg, 1.26 mmol, 219.16 uL). The reaction mixture was stirred for 16 hr at 25° C. The reaction mixture was diluted with water (10 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[4-[7-[2-(2,6-dioxo-3-piperidyl)-1-oxo-isoindolin-4-yl]heptylcarbamoyl]-3-fluoro-phenyl]-7-fluoro-4-(methylamino)quinoline-3-carboxamide (Compound 402, 10 mg, 13.57 μmol, 10.79% yield) as a white solid. ¹H NMR (400 MHz, DMSO-d₆) δ 10.99 (s, 1H), 8.58 (d, J=4.2 Hz, 1H), 8.52 (d, J=8.3 Hz, 1H), 8.41-8.32 (m, 2H), 8.06 (s, 1H), 7.72 (t, J=7.8 Hz, 1H), 7.66-7.53 (m, 3H), 7.48-7.42 (m, 2H), 5.13 (dd, J=13.2, 5.2 Hz, 1H), 4.47 (d, J=17.2 Hz, 1H), 4.31 (d, J=17.1 Hz, 1H), 3.28-3.22 (m, 2H), 3.01 (d, J=4.9 Hz, 3H), 2.96-2.80 (m, 2H), 2.69-2.59 (m, 3H), 2.44-2.38 (m, 1H), 2.05-1.97 (m, 1H), 1.68-1.57 (m, 2H), 1.57-1.47 (m, 2H), 1.35 (s, 5H), 0.74-0.66 (m, 2H), 0.56 (p, J=4.5 Hz, 2H). LCMS (ES+): m/z 737 [M+H]⁺

### Synthesis of Compound 403

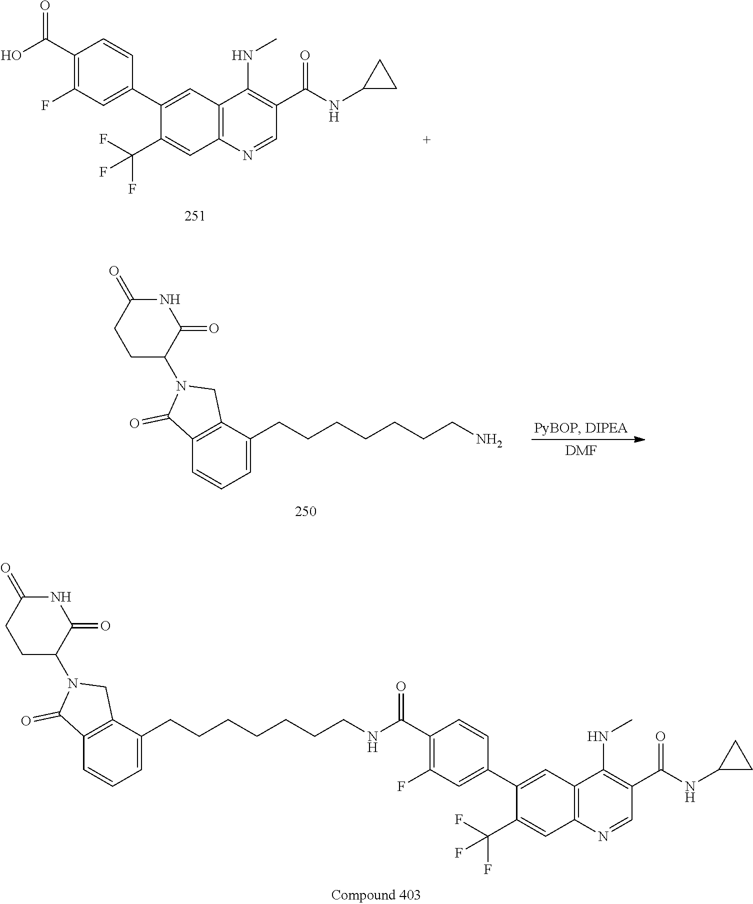

To a stirred solution of 4-[3-(cyclopropylcarbamoyl)-4-(methylamino)-7-(trifluoromethyl)-6-quinolyl]-2-fluoro-benzoic acid (251, 50 mg, 111.76 μmol) in DMF (5 mL) was added 3-[4-(7-aminoheptyl)-1-oxo-isoindolin-2-yl]piperidine-2,6-dione (250, 47.94 mg, 134.11 μmol), PyBOP (87.24 mg, 167.64 μmol), and DIPEA (144.44 mg, 1.12 mmol, 194.67 uL). The reaction mixture was stirred for 16 hr at 25° C. The reaction mixture was diluted with water (10 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[4-[7-[2-(2,6-dioxo-3-piperidyl)-1-oxo-isoindolin-4-yl]heptylcarbamoyl]-3-fluoro-phenyl]-4-(methylamino)-7-(trifluoromethyl)quinoline-3-carboxamide (Compound 403, 10 mg, 12.66 μmol, 11.33% yield) as a white solid. ¹H NMR (400 MHz, DMSO-d₆) δ 11.00 (s, 1H), 8.64-8.61 (m, 1H), 8.46-8.41 (m, 2H), 8.33 (s, 1H), 8.19 (s, 1H), 7.88-7.80 (m, 1H), 7.67 (td, J=7.8, 1.8 Hz, 1H), 7.58-7.54 (m, 1H), 7.49-7.43 (m, 2H), 7.37 (d, J=10.7 Hz, 1H), 7.31 (d, J=8.0 Hz, 1H), 5.13 (dd, J=13.0, 5.0 Hz, 1H), 4.47 (d, J=17.3 Hz, 1H), 4.31 (d, J=16.9 Hz, 1H), 3.29-3.21 (m, 2H), 2.97-2.91 (m, 3H), 2.90-2.80 (m, 2H), 2.64 (d, J=9.2 Hz, 3H), 2.44-2.36 (m, 1H), 2.06-1.95 (m, 1H), 1.69-1.58 (m, 2H), 1.57-1.49 (m, 2H), 1.35 (s, 6H), 0.75-0.68 (m, 2H), 0.59-0.51 (m, 2H). LCMS (ES+): m/z 787 [M+H]⁺

### Synthesis of Compound 404

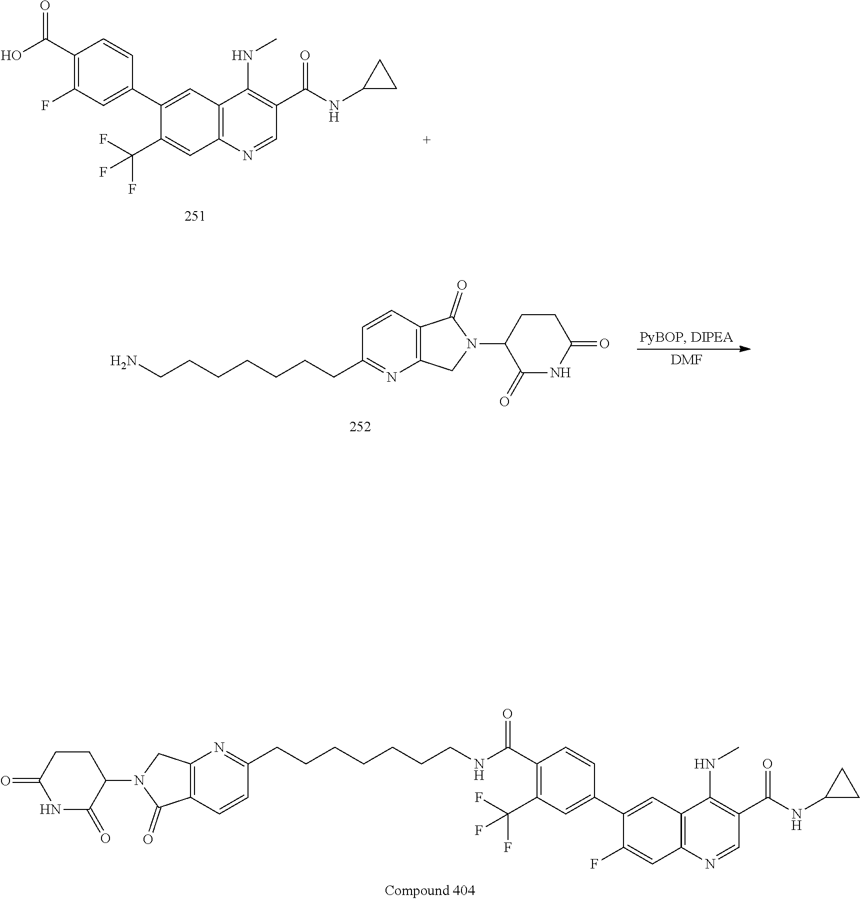

To a solution of 4-[3-(cyclopropylcarbamoyl)-4-(methylamino)-7-(trifluoromethyl)-6-quinolyl]-2-fluoro-benzoic acid (251, 50 mg, 111.76 μmol) and 3-[2-(7-aminoheptyl)-5-oxo-7H-pyrrolo[3,4-b]pyridin-6-yl]piperidine-2,6-dione (252, 48.07 mg, 134.11 μmol) in DMF (5 mL) was added DIPEA (72.22 mg, 558.81 μmol, 97.33 uL) and PyBOP (87.24 mg, 167.64 μmol). The resulting mixture was stirred for 16 hr at 25° C. Added water (20 mL) to the reaction mixture and extracted with ethyl acetate (3×25 mL). The combined organic extracts were dried over anhydrous sodium sulfate, filtered and evaporated the solvent completely under reduced pressure. The resulting crude was purified by Reverse phase prep HPLC to yield N-cyclopropyl-6-[4-[7-[6-(2,6-dioxo-3-piperidyl)-5-oxo-7H-pyrrolo[3,4-b]pyridin-2-yl]heptylcarbamoyl]-3-(trifluoromethyl)phenyl]-7-fluoro-4-(methylamino)quinoline-3-carboxamide (Compound 404, 20 mg, 23.91 μmol, 21.40% yield) as an off white solid. ¹H NMR (400 MHz, Methanol-d₄) δ 8.46 (s, 1H), 8.26 (s, 1H), 8.22 (s, 1H), 8.08 (d, J=8.0 Hz, 1H), 7.77 (t, J=7.7 Hz, 1H), 7.46 (d, J=8.0 Hz, 1H), 7.37-7.27 (m, 2H), 5.19 (dd, J=13.4, 5.2 Hz, 1H), 4.53 (d, J=17.5 Hz, 1H), 4.47 (d, J=17.5 Hz, 1H), 3.45-3.38 (m, 2H), 3.09 (s, 3H), 2.97-2.84 (m, 4H), 2.77 (ddd, J=17.6, 4.7, 2.4 Hz, 1H), 2.51 (qd, J=13.2, 4.7 Hz, 1H), 2.18 (dtd, J=12.8, 5.3, 2.4 Hz, 1H), 1.86-1.76 (m, 2H), 1.70-1.60 (m, 2H), 1.44 (s, 6H), 0.88-0.81 (m, 2H), 0.70-0.63 (m, 2H). LCMS (ES+): m/z 788 [M+H]⁺

### Synthesis of Compound 405

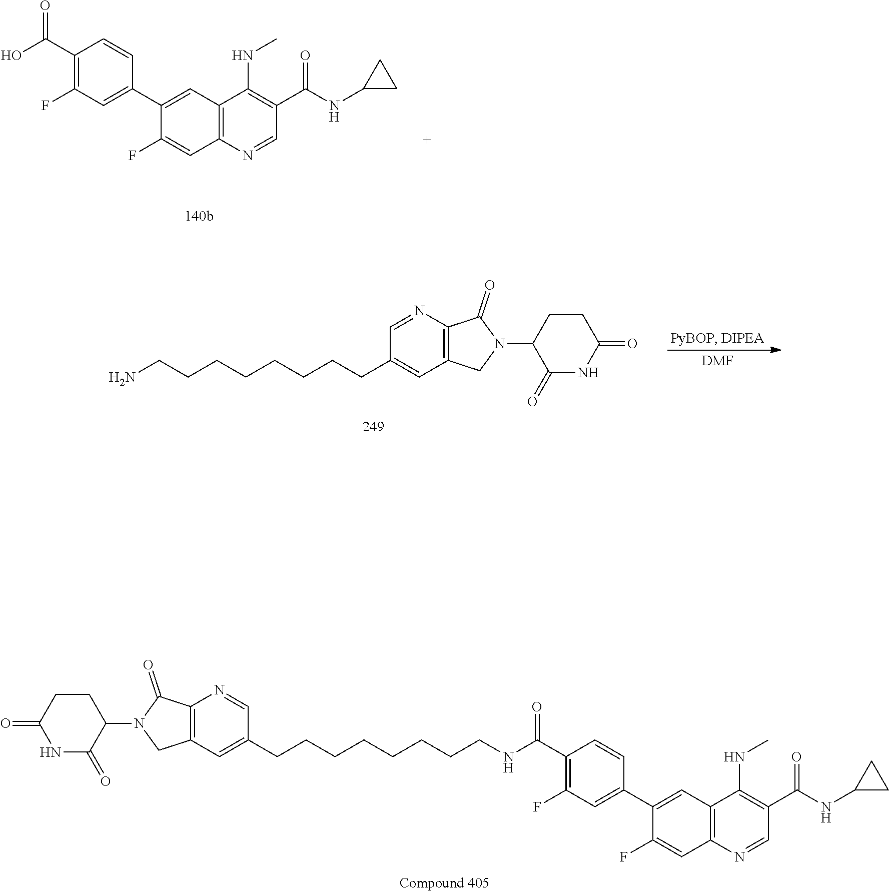

To a solution of 4-[3-(cyclopropylcarbamoyl)-7-fluoro-4-(methylamino)-6-quinolyl]-2-fluoro-benzoic acid (140b, 50 mg, 125.83 μmol) and 3-[3-(8-aminooctyl)-7-oxo-5H-pyrrolo[3,4-b]pyridin-6-yl]piperidine-2,6-dione (249, 56.24 mg, 150.99 μmol) in DMF (5 mL) was added DIPEA (81.31 mg, 629.13 μmol, 109.58 uL) and PyBOP (98.22 mg, 188.74 μmol). The resulting mixture was stirred for 16 hr at 25° C. Added water (20 mL) to the reaction mixture and extracted with ethyl acetate (3×25 mL). The combined organic extracts were dried over anhydrous sodium sulfate, filtered and evaporated the solvent completely under reduced pressure. The resulting crude was purified by Reverse phase prep HPLC to yield N-cyclopropyl-6-[4-[8-[6-(2,6-dioxo-3-piperidyl)-7-oxo-5H-pyrrolo[3,4-b]pyridin-3-yl]octylcarbamoyl]-3-fluoro-phenyl]-7-fluoro-4-(methylamino)quinoline-3-carboxamide (Compound 405, 25 mg, 32.87 μmol, 26.12% yield) as an off white solid. ¹H NMR (400 MHz, Methanol-d₄) δ 8.58 (d, J=1.8 Hz, 1H), 8.46 (d, J=8.0 Hz, 1H), 8.37 (s, 1H), 7.91 (d, J=1.8 Hz, 1H), 7.80 (t, J=7.9 Hz, 1H), 7.61-7.51 (m, 3H), 5.18 (dd, J=13.3, 5.2 Hz, 1H), 4.53 (d, J=17.1 Hz, 1H), 4.47 (d, J=17.3 Hz, 1H), 3.41 (t, J=7.0 Hz, 2H), 3.35 (s, 1H), 3.15 (s, 3H), 2.98-2.85 (m, 2H), 2.85-2.73 (m, 3H), 2.50 (qd, J=13.2, 4.7 Hz, 1H), 2.23-2.12 (m, 1H), 1.78-1.59 (m, 4H), 1.41 (s, 7H), 0.84 (td, J=7.1, 5.1 Hz, 2H), 0.69-0.63 (m, 2H). LCMS (ES+): m/z 752 [M+H]⁺

### Synthesis of Compound 406

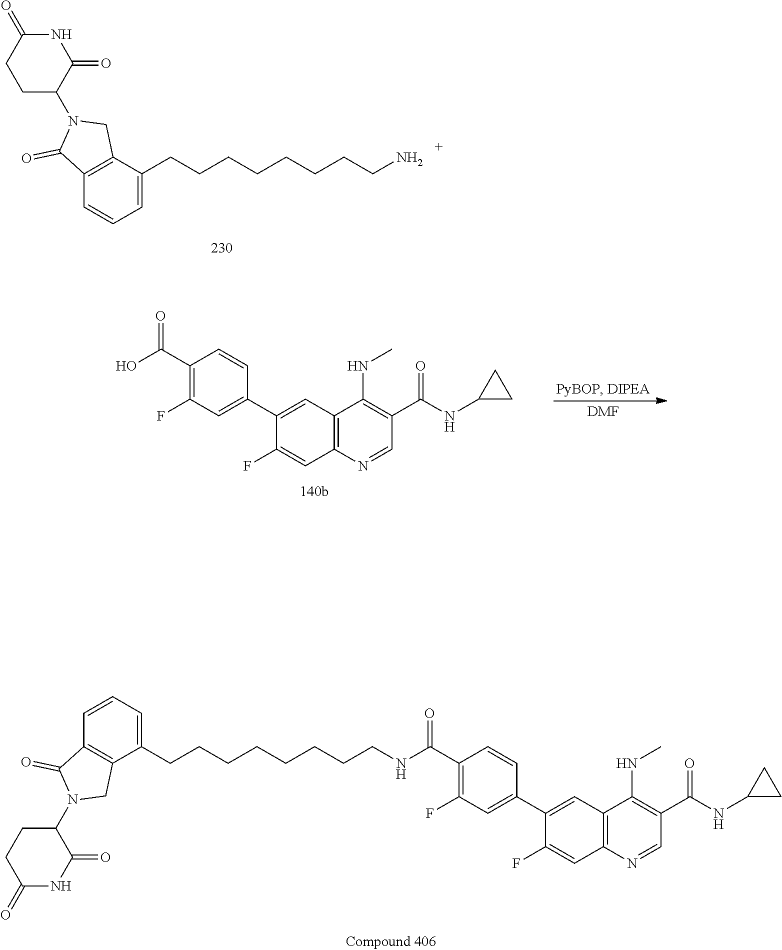

To a stirred solution of 3-[4-(8-aminooctyl)-1-oxo-isoindolin-2-yl]piperidine-2,6-dione (230, 50 mg, 134.60 μmol), 4-[3-(cyclopropylcarbamoyl)-7-fluoro-4-(methylamino)-6-quinolyl]-2-fluoro-benzoic acid (140b, 80.23 mg, 201.90 μmol) in DMF (5 mL) was added PyBOP (105.07 mg, 201.90 μmol) and DIPEA (86.98 mg, 673.00 μmol, 117.22 uL). The reaction mixture was stirred for 16 hr at 25° C. The reaction mixture was evaporated completely under reduced pressure, the resulting crude was purified by reverse phase preparative HPLC to yield N-cyclopropyl-6-[4-[8-[2-(2,6-dioxo-3-piperidyl)-1-oxo-isoindolin-4-yl]octylcarbamoyl]-3-fluoro-phenyl]-7-fluoro-4-(methylamino)quinoline-3-carboxamide (Compound 406, 23 mg, 30.26 μmol, 22.48% yield) as a yellow color solid. ¹H NMR (400 MHz, DMSO-d₆) δ 10.99 (s, 1H), 9.49 (s, 1H), 8.87 (d, J=4.1 Hz, 1H), 8.73 (d, J=7.5 Hz, 1H), 8.66 (s, 1H), 8.41 (t, J=5.7 Hz, 1H), 7.79-7.73 (m, 2H), 7.65 (d, J=11.3 Hz, 1H), 7.60 (dt, J=7.8, 1.6 Hz, 1H), 7.57-7.53 (m, 1H), 7.46 (s, 1H), 7.45 (d, J=2.1 Hz, 1H), 5.13 (dd, J=13.3, 5.1 Hz, 1H), 4.46 (d, J=17.3 Hz, 1H), 4.30 (d, J=17.1 Hz, 1H), 3.26 (q, J=6.5 Hz, 2H), 3.10 (s, 2H), 3.01 (td, J=6.7, 4.0 Hz, 2H), 2.97-2.80 (m, 2H), 2.65-2.59 (m, 2H), 2.46-2.37 (m, 1H), 2.06-1.95 (m, 1H), 1.77-1.70 (m, 2H), 1.66-1.57 (m, 2H), 1.57-1.48 (m, 2H), 1.32 (s, 6H), 0.76 (td, J=7.1, 4.8 Hz, 2H), 0.63-0.56 (m, 2H). LCMS (ES+): m/z 751 [M+H]⁺

### Synthesis of Compound 407

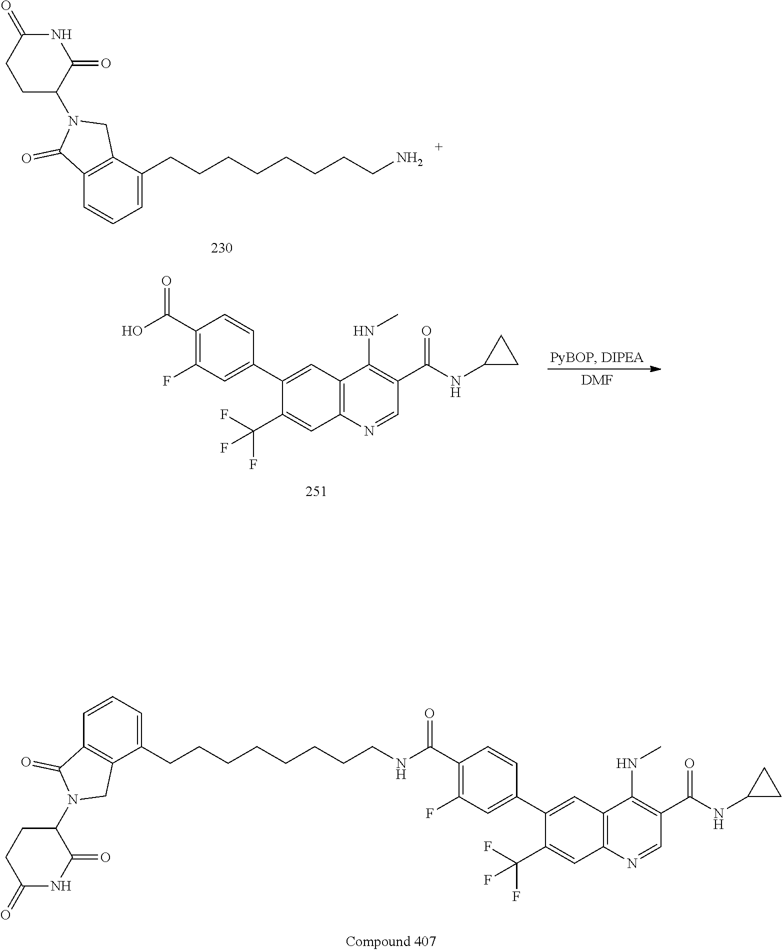

To a stirred solution of 3-[4-(8-aminooctyl)-1-oxo-isoindolin-2-yl]piperidine-2,6-dione (230, 50 mg, 134.60 μmol), 4-[3-(cyclopropylcarbamoyl)-4-(methylamino)-7-(trifluoromethyl)-6-quinolyl]-2-fluoro-benzoic acid (251, 90.33 mg, 201.90 μmol) in DMF (5 mL) was added PyBOP (105.07 mg, 201.90 μmol) and DIPEA (86.98 mg, 673.00 μmol, 117.22 uL). The reaction mixture was stirred for 16 hr at 25° C. The reaction mixture solvent was evaporated the completely under reduced pressure. The resulting crude was purified by reverse phase preparative HPLC to yield N-cyclopropyl-6-[4-[8-[2-(2,6-dioxo-3-piperidyl)-1-oxo-isoindolin-4-yl]octylcarbamoyl]-3-fluoro-phenyl]-4-(methylamino)-7-(trifluoromethyl)quinoline-3-carboxamide (Compound 407, 26 mg, 32.14 μmol, 23.88% yield) as a yellow color solid. ¹H NMR (400 MHz, DMSO-d₆) δ 10.99 (s, 1H), 9.40 (s, 1H), 8.88 (d, J=4.1 Hz, 1H), 8.76 (s, 1H), 8.55 (s, 1H), 8.45 (t, J=5.7 Hz, 1H), 8.32 (s, 1H), 7.71 (t, J=7.7 Hz, 1H), 7.58-7.53 (m, 1H), 7.46 (s, 1H), 7.45 (d, J=3.2 Hz, 1H), 7.39 (d, J=10.9 Hz, 1H), 7.32 (d, J=7.8 Hz, 1H), 5.13 (dd, J=13.2, 5.2 Hz, 1H), 4.46 (d, J=17.2 Hz, 1H), 4.38-4.26 (m, 1H), 3.26 (q, J=6.5 Hz, 2H), 3.08 (s, 3H), 3.03-2.80 (m, 4H), 2.66-2.60 (m, 2H), 2.43-2.38 (m, 1H), 2.05-1.96 (m, 1H), 1.76-1.70 (m, 1H), 1.65-1.57 (m, 2H), 1.57-1.47 (m, 2H), 1.32 (s, 7H), 0.75 (td, J=7.1, 4.8 Hz, 2H), 0.59 (dt, J=7.5, 4.6 Hz, 2H). LCMS (ES+): m/z 801 [M+H]⁺

### Synthesis of Compound 408

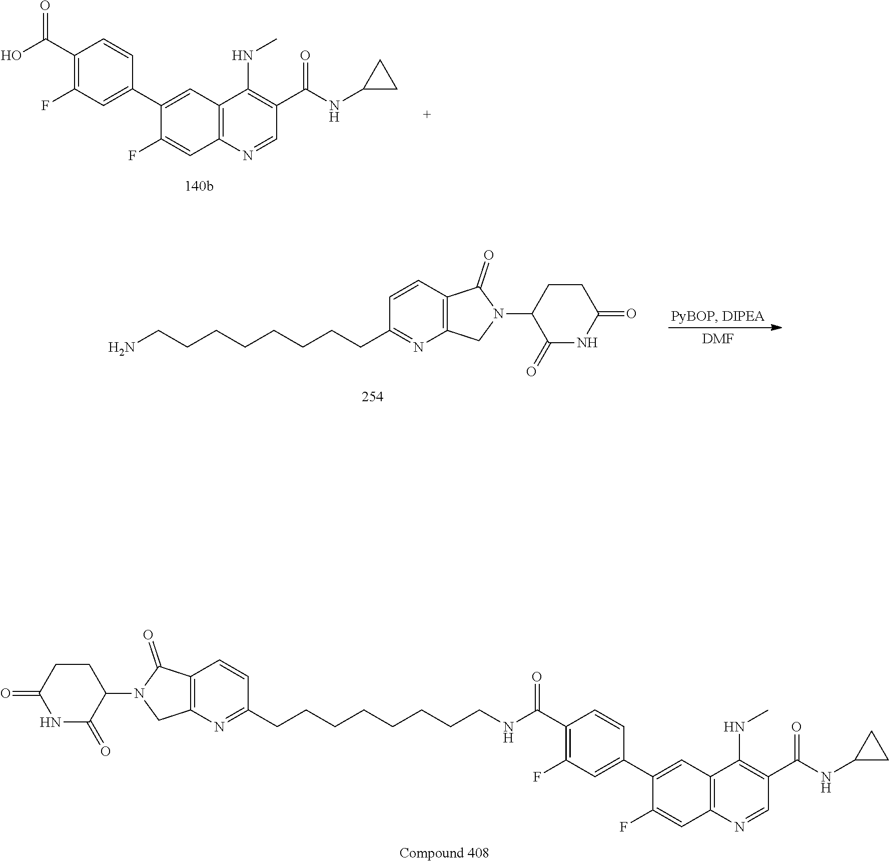

To a stirred solution of 4-[3-(cyclopropylcarbamoyl)-7-fluoro-4-(methylamino)-6-quinolyl]-2-fluoro-benzoic acid (140b, 30 mg, 75.50 μmol), 3-[2-(8-aminooctyl)-5-oxo-7H-pyrrolo[3,4-b]pyridin-6-yl]piperidine-2,6-dione (254, 33.74 mg, 90.59 μmol) in DMF (5 mL) was added PyBOP (58.93 mg, 113.24 μmol) and DIPEA (97.57 mg, 754.96 μmol, 131.50 uL). The reaction mixture was stirred for 16 hr at 25° C. The solvent was evaporated completely under reduced pressure, and the resulting crude was purified by reverse phase preparative HPLC to yield N-cyclopropyl-6-[4-[8-[6-(2,6-dioxo-3-piperidyl)-5-oxo-7H-pyrrolo[3,4-b]pyridin-2-yl]octylcarbamoyl]-3-fluoro-phenyl]-7-fluoro-4-(methylamino)quinoline-3-carboxamide (Compound 408, 5 mg, 5.65 μmol, 7.49% yield) as a light yellow solid. ¹H NMR (400 MHz, Methanol-d₄) δ 8.67 (d, J=7.2 Hz, 1H), 8.55 (s, 1H), 8.08 (d, J=8.0 Hz, 1H), 7.84 (dd, J=8.6, 6.8 Hz, 1H), 7.68 (d, J=10.5 Hz, 1H), 7.65-7.55 (m, 2H), 7.45 (d, J=8.0 Hz, 1H), 5.18 (dd, J=13.4, 5.2 Hz, 1H), 4.53 (d, J=17.6 Hz, 1H), 4.46 (d, J=17.5 Hz, 1H), 3.41 (t, J=7.0 Hz, 2H), 2.98-2.84 (m, 4H), 2.83-2.74 (m, 1H), 2.51 (qd, J=13.2, 4.7 Hz, 1H), 2.24-2.17 (m, 1H), 1.85-1.73 (m, 2H), 1.63 (d, J=7.1 Hz, 2H), 1.40 (s, 8H), 1.34-1.17 (m, 2H), 0.91-0.83 (m, 2H), 0.72-0.64 (m, 2H). LCMS (ES+): m/z 752 [M+H]⁺

### Synthesis of Compound 409

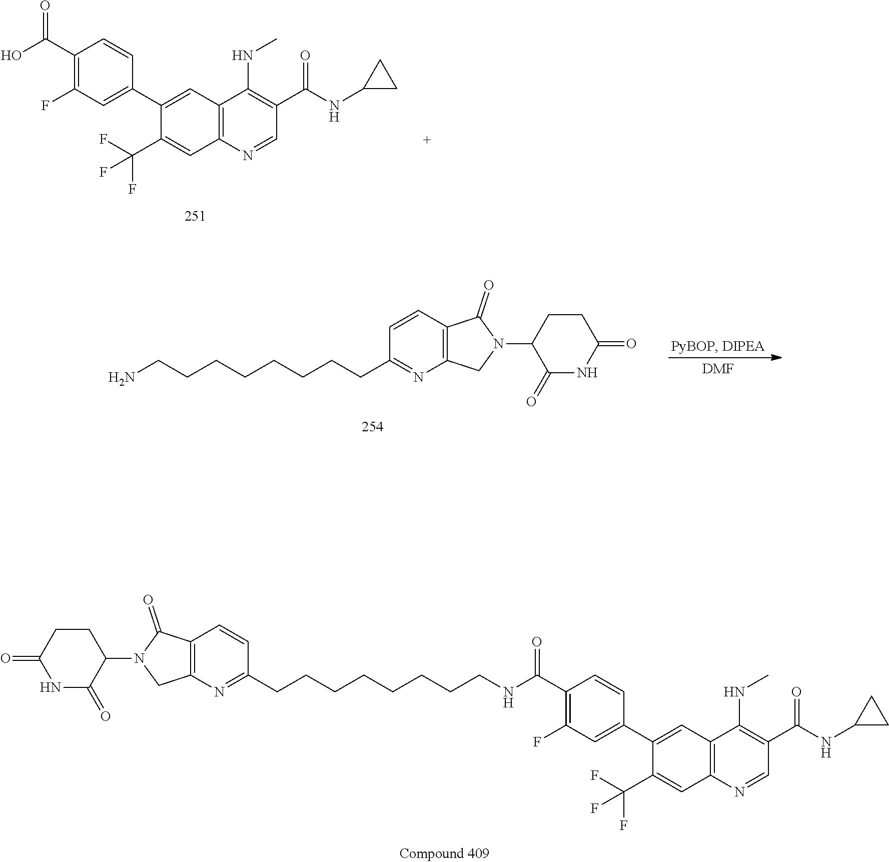

To a solution of 4-[3-(cyclopropylcarbamoyl)-4-(methylamino)-7-(trifluoromethyl)-6-quinolyl]-2-fluoro-benzoic acid (251, 50 mg, 111.76 μmol) and 3-[2-(8-aminooctyl)-5-oxo-7H-pyrrolo[3,4-b]pyridin-6-yl]piperidine-2,6-dione (254, 49.95 mg, 134.11 μmol) in DMF (5 mL) was added DIPEA (72.22 mg, 558.81 μmol, 97.33 uL) and PyBOP (87.24 mg, 167.64 μmol). The resulting mixture was stirred for 16 hr at 25° C. Added water (20 mL) to the reaction mixture and extracted with ethyl acetate (3×25 mL). The combined organic extracts were dried over anhydrous sodium sulfate, filtered and evaporated the solvent completely under reduced pressure. The resulting crude was purified by Reverse phase prep HPLC to yield N-cyclopropyl-6-[4-[8-[6-(2,6-dioxo-3-piperidyl)-5-oxo-7H-pyrrolo[3,4-b]pyridin-2-yl]octylcarbamoyl]-3-fluoro-phenyl]-4-(methylamino)-7-(trifluoromethyl)quinoline-3-carboxamide (Compound 409, 15 mg, 18.66 μmol, 16.70% yield) as an off white solid. ¹H NMR (400 MHz, Methanol-d₄) δ 8.46 (s, 1H), 8.26 (s, 1H), 8.22 (s, 1H), 8.08 (d, J=8.0 Hz, 1H), 7.77 (t, J=7.7 Hz, 1H), 7.45 (d, J=7.9 Hz, 1H), 7.32 (dd, J=14.5, 9.7 Hz, 2H), 5.19 (dd, J=13.3, 5.2 Hz, 1H), 4.53 (d, J=17.5 Hz, 1H), 4.46 (d, J=17.4 Hz, 1H), 3.41 (t, J=7.1 Hz, 2H), 3.09 (s, 3H), 2.96-2.85 (m, 4H), 2.78 (ddd, J=17.6, 4.7, 2.3 Hz, 1H), 2.51 (qd, J=13.2, 4.7 Hz, 1H), 2.19 (dtd, J=12.9, 5.3, 2.4 Hz, 1H), 1.84-1.74 (m, 2H), 1.70-1.59 (m, 2H), 1.40 (d, J=2.7 Hz, 8H), 0.84 (td, J=7.1, 5.1 Hz, 2H), 0.69-0.63 (m, 2H). LCMS (ES+): m/z 802 [M+H]⁺

### Synthesis of Compound 410

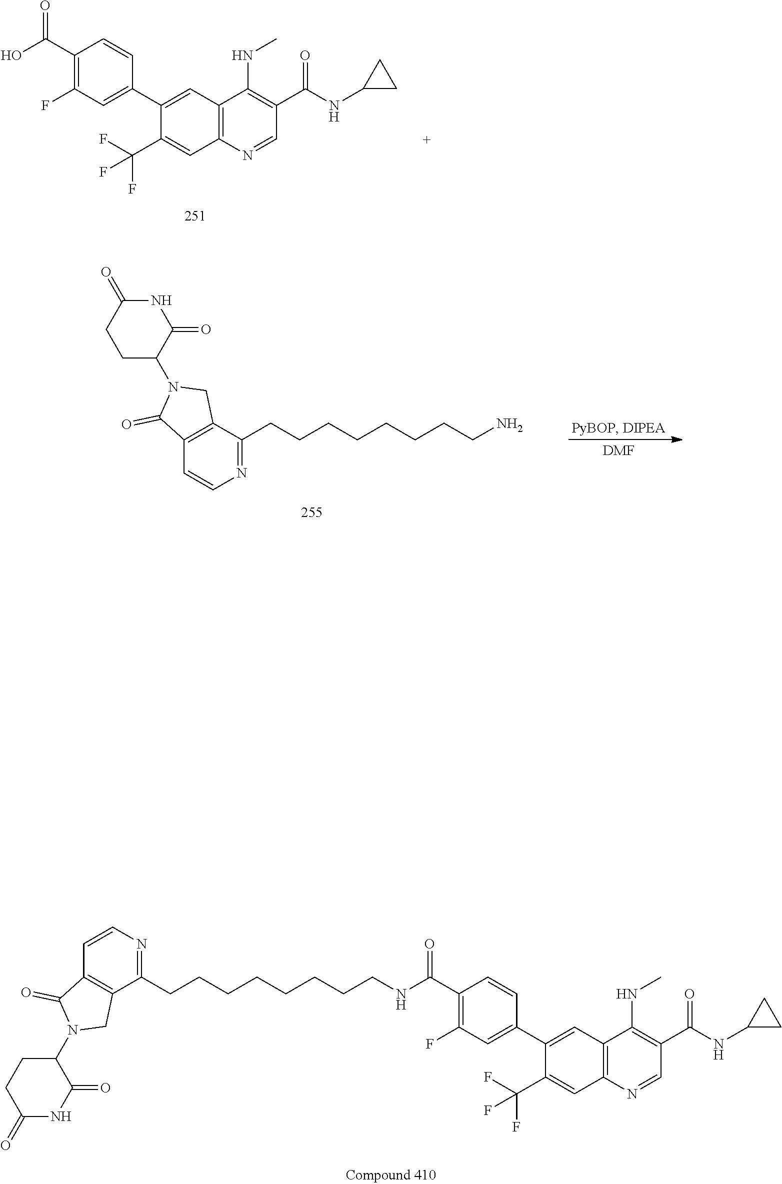

To a stirred solution of 4-[3-(cyclopropylcarbamoyl)-4-(methylamino)-7-(trifluoromethyl)-6-quinolyl]-2-fluoro-benzoic acid (251, 50 mg, 111.76 μmol) in DMF (5 mL) was added 3-[4-(8-aminooctyl)-1-oxo-3H-pyrrolo[3,4-c]pyridin-2-yl]piperidine-2,6-dione (255, 49.95 mg, 134.11 PyBOP (87.24 mg, 167.64 μmop, and DIPEA (144.44 mg, 1.12 mmol, 194.67 uL). The reaction mixture was stirred for 16 hr at 25° C. The reaction mixture was diluted with water (10 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[4-[8-[2-(2,6-dioxo-3-piperidyl)-1-oxo-3H-pyrrolo[3,4-c]pyridin-4-yl]octylcarbamoyl]-3-fluoro-phenyl]-4-(methylamino)-7-(trifluoromethyl)quinoline-3-carboxamide (Compound 410, 10 mg, 12.33 μmol, 11.04% yield) as a white solid. ¹H NMR (400 MHz, DMSO-d₆) δ 11.04 (s, 1H), 9.55 (s, 1H), 8.91 (d, J=4.1 Hz, 1H), 8.80 (s, 1H), 8.68 (d, J=5.0 Hz, 1H), 8.57 (s, 1H), 8.48-8.43 (m, 1H), 8.34 (s, 1H), 7.72 (t, J=7.7 Hz, 1H), 7.57 (d, J=5.0 Hz, 1H), 7.40 (d, J=10.8 Hz, 1H), 7.33 (d, J=8.0 Hz, 1H), 5.17 (dd, J=13.3, 5.2 Hz, 1H), 4.59 (d, J=17.8 Hz, 1H), 4.43 (d, J=17.8 Hz, 1H), 3.26 (q, J=6.7 Hz, 2H), 3.15-3.04 (m, 3H), 2.96-2.78 (m, 4H), 2.64-2.53 (m, 2H), 2.43-2.39 (m, 1H), 2.09-1.97 (m, 1H), 1.80-1.68 (m, 2H), 1.59-1.46 (m, 2H), 1.33 (s, 6H), 0.75 (dt, J=6.9, 3.3 Hz, 2H), 0.63-0.55 (m, 2H). LCMS (ES+): m/z 802 [M+H]⁺

### Synthesis of Compound 411

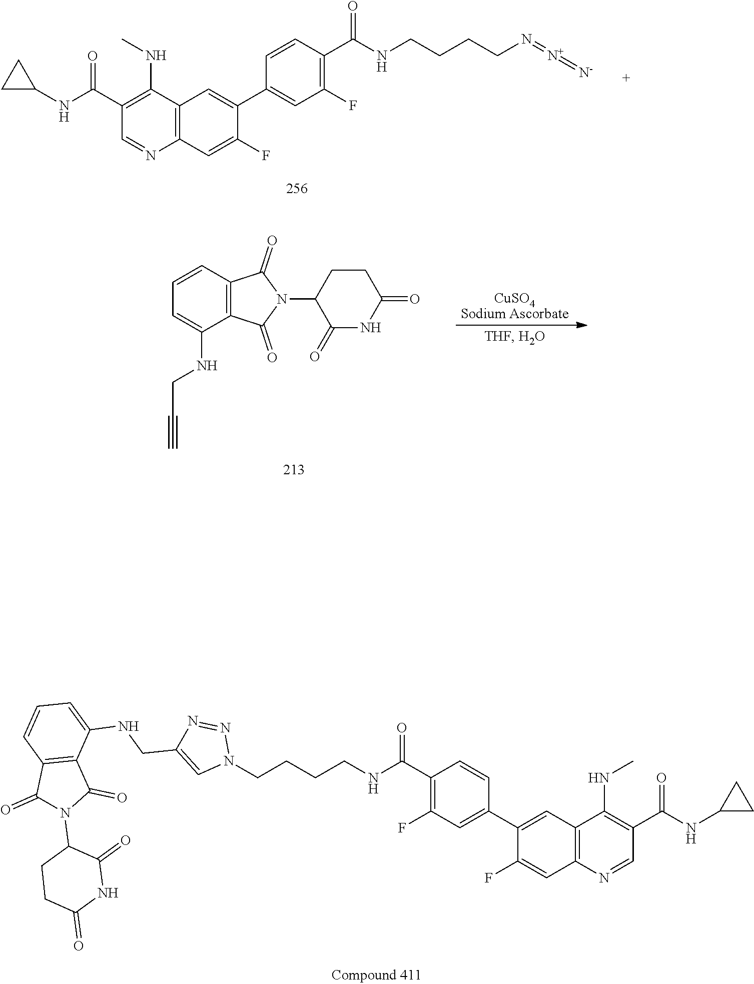

To a solution of 6-[4-(4-azidobutylcarbamoyl)-3-fluoro-phenyl]-N-cyclopropyl-7-fluoro-4-(methylamino)quinoline-3-carboxamide (256, 47.56 mg, 96.37 μmol) and 2-(2,6-dioxo-3-piperidyl)-4-(prop-2-ynylamino)isoindoline-1,3-dione (213, 30 mg, 96.37 μmol) in THF (5 mL) and Water (1 mL) was added Copper (II) sulfate (30.76 mg, 192.75 μmol, 8.55 uL) and (+)-Sodium L-ascorbate (38.18 mg, 192.75 μmol). The resulting mixture was stirred for 16 hr at rt. The mixture was diluted with water and extracted with ethyl acetate (3×15 mL). The combined organic extracts were washed with water and brine solution, dried over anhydrous sodium sulfate, filtered and evaporated under reduced pressure. The resulting crude was purified by reverse phase prep HPLC to yield N-cyclopropyl-6-[4-[4-[4-[[[2-(2,6-dioxo-3-piperidyl)-1,3-dioxo-isoindolin-4-yl]amino]methyl]triazol-1-yl]butylcarbamoyl]-3-fluoro-phenyl]-7-fluoro-4-(methylamino)quinoline-3-carboxamide (Compound 411, 60 mg, 73.62 μmol, 76.39% yield) as an yellow solid. ¹H NMR (400 MHz, DMSO-d₆) δ 11.10 (s, 1H), 9.52 (s, 1H), 8.88 (d, J=4.1 Hz, 1H), 8.74 (d, J=7.4 Hz, 1H), 8.67 (s, 1H), 8.51-8.42 (m, 1H), 8.04 (s, 1H), 7.81-7.73 (m, 2H), 7.66 (d, J=11.3 Hz, 1H), 7.58 (dd, J=16.6, 8.1 Hz, 2H), 7.16 (d, J=8.6 Hz, 1H), 7.08 (s, 1H), 7.04 (d, J=7.1 Hz, 1H), 5.05 (dd, J=12.9, 5.3 Hz, 1H), 4.60 (d, J=5.6 Hz, 2H), 4.38 (t, J=7.1 Hz, 2H), 3.28 (q, J=6.6 Hz, 2H), 3.10 (s, 3H), 2.94-2.80 (m, 2H), 2.64-2.55 (m, 2H), 2.06-1.95 (m, 1H), 1.86 (p, J=6.8 Hz, 2H), 1.54-1.41 (m, 2H), 0.76 (td, J=7.0, 4.7 Hz, 2H), 0.60 (p, J=4.5 Hz, 2H). LCMS (ES+): m/z 805 [M+H]⁺

### Synthesis of Compound 412

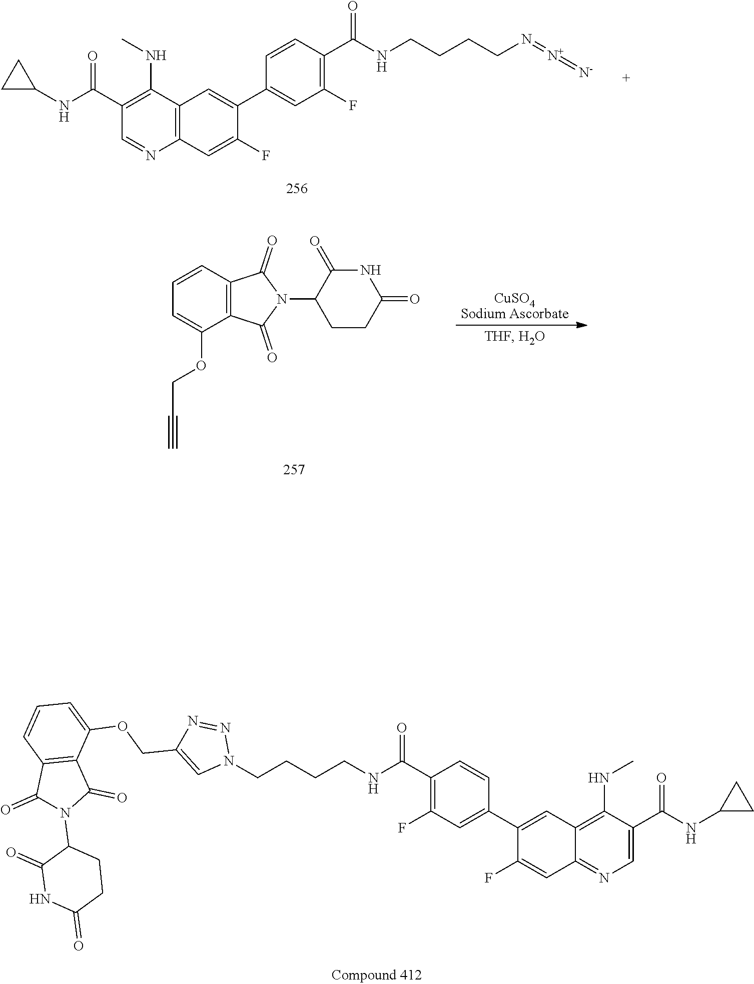

To a stirred solution of 6-[4-(4-azidobutylcarbamoyl)-3-fluoro-phenyl]-N-cyclopropyl-7-fluoro-4-(methylamino)quinoline-3-carboxamide (256, 50 mg, 101.32 μmol) in Water (1 mL) and THF (4 mL) was added 2-(2,6-dioxo-3-piperidyl)-4-prop-2-ynoxy-isoindoline-1,3-dione (257, 37.97 mg, 121.58 μmol), copper sulfate (32.34 mg, 202.63 μmol, 8.98 uL) and (+)-Sodium L-ascorbate (40.14 mg, 202.63 μmol) at room temperature. The reaction mixture was stirred for 16 h at room temperature. Water was added to the reaction mixture and then extracted with ethyl acetate. The combined organics were dried over anhydrous sodium sulfate and then concentrated to yield the crude, which was purified by Prep-HPLC to yield N-cyclopropyl-6-[4-[4-[4-[[2-(2,6-dioxo-3-piperidyl)-1,3-dioxo-isoindolin-4-yl]oxymethyl]triazol-1-yl]butylcarbamoyl]-3-fluoro-phenyl]-7-fluoro-4-(methylamino)quinoline-3-carboxamide (Compound 412, 20 mg, 24.69 μmol, 24.37% yield) as light yellow colored solid. LCMS (ES+): m/z 806 [M+H]⁺

### Synthesis of Compound 413

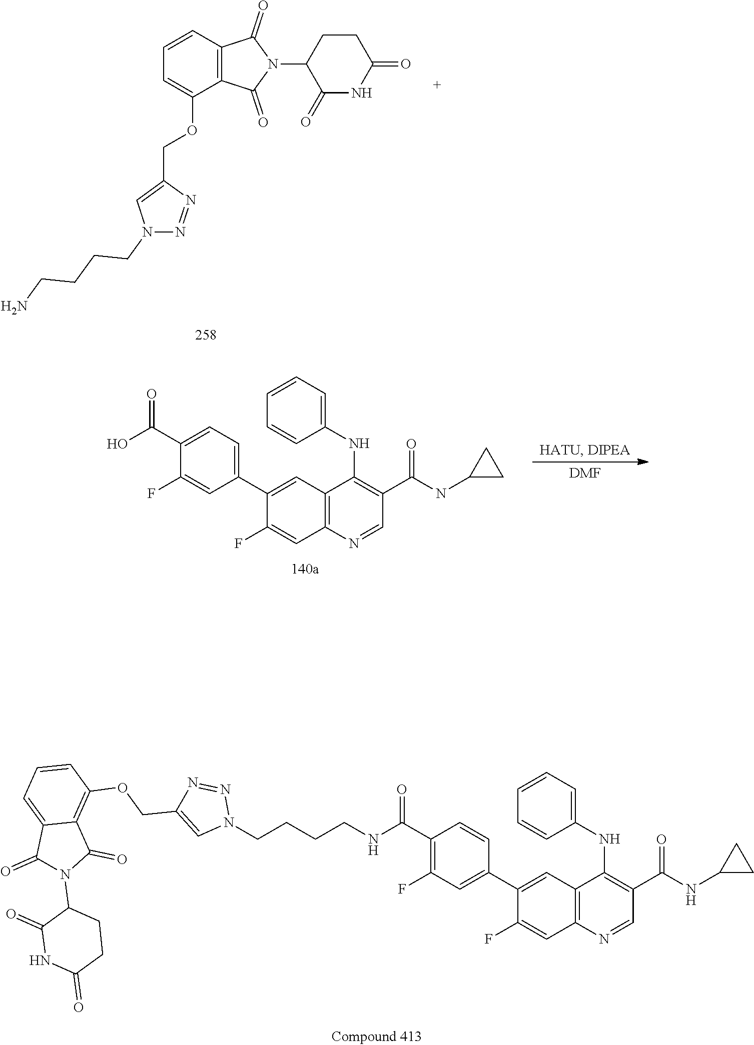

To a stirred solution of 4-[[1-(4-aminobutyl)triazol-4-yl]methoxy]-2-(2,6-dioxo-3-piperidyl)isoindoline-1,3-dione (258, 510.47 mg, 1.20 mmol) and 4-[4-anilino-3-(cyclopropylcarbamoyl)-7-fluoro-6-quinolyl]-2-fluoro-benzoic acid (140a, 500 mg, 1.09 mmol) in DMF (15 mL) was added DIPEA (703.26 mg, 5.44 mmol, 947.79 uL) and HATU (620.69 mg, 1.63 mmol). The resulting solution was stirred for 16 hr at 25° C. Added water (50 mL) to the mixture and extracted with ethyl acetate (3×30 mL). The combined organic extracts were washed with water and brine solution, dried over anhydrous sodium sulfate, filtered and evaporated the solvent completely under reduced pressure. The resulting crude was purified by reverse phase prep HPLC to yield 4-anilino-N-cyclopropyl-6-[4-[4-[4-[[2-(2,6-dioxo-3-piperidyl)-1,3-dioxo-isoindolin-4-yl]oxymethyl]triazol-1-yl]butylcarbamoyl]-3-fluoro-phenyl]-7-fluoro-quinoline-3-carboxamide (Compound 413, 120 mg, 133.06 μmol, 12.23% yield) as an yellow solid. ¹H NMR (400 MHz, DMSO-d₆) δ 11.10 (s, 1H), 10.03 (s, 1H), 8.79 (s, 1H), 8.53 (d, J=3.6 Hz, 1H), 8.47-8.40 (m, 1H), 8.29 (s, 1H), 8.26 (d, J=8.5 Hz, 1H), 7.87-7.79 (m, 2H), 7.74 (d, J=8.6 Hz, 1H), 7.66 (t, J=7.8 Hz, 1H), 7.47 (d, J=7.2 Hz, 1H), 7.41-7.27 (m, 4H), 7.14-7.01 (m, 3H), 5.42 (s, 2H), 5.07 (dd, J=12.7, 5.4 Hz, 1H), 4.43 (t, J=7.0 Hz, 2H), 3.29 (d, J=8.7 Hz, 2H), 2.87 (ddd, J=17.6, 14.2, 5.5 Hz, 1H), 2.62-2.55 (m, 1H), 2.05-1.96 (m, 1H), 1.88 (p, J=7.2 Hz, 2H), 1.49 (p, J=7.1 Hz, 2H), 1.29-1.22 (m, 1H), 0.58-0.50 (m, 2H), 0.33-0.25 (m, 2H). LCMS (ES+): m/z 868 [M+H]⁺

### Synthesis of Compound 414

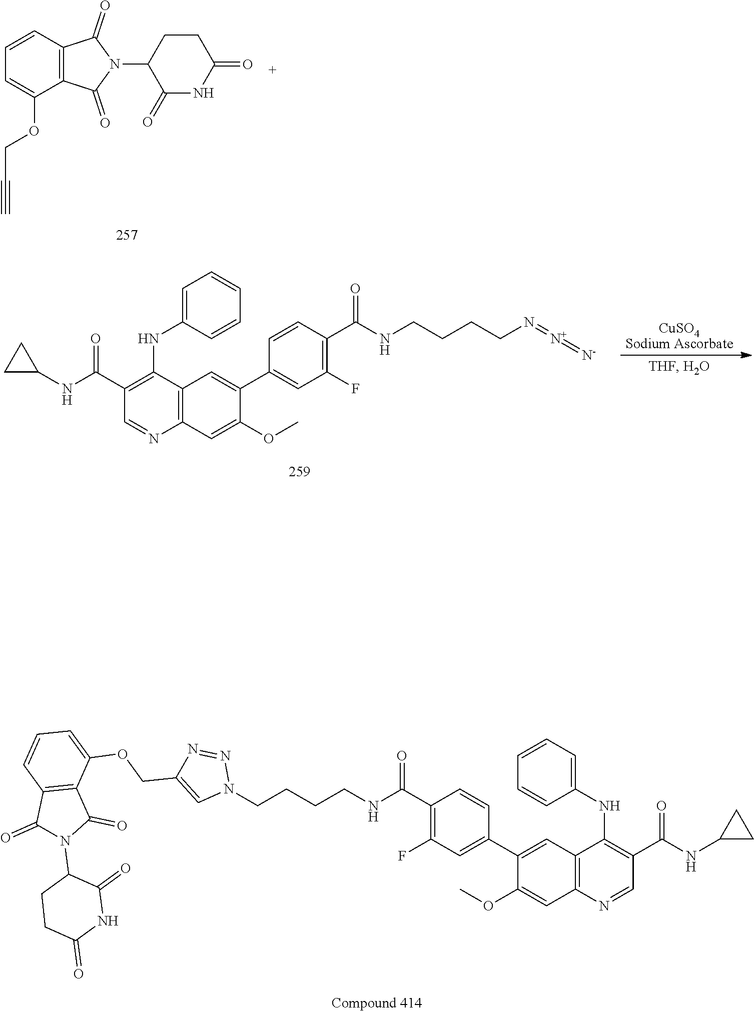

To a solution of 2-(2,6-dioxo-3-piperidyl)-4-prop-2-ynoxy-isoindoline-1,3-dione (257, 50 mg, 160.11 μmol) and 4-anilino-6-[4-(4-azidobutylcarbamoyl)-3-fluoro-phenyl]-N-cyclopropyl-7-methoxy-quinoline-3-carboxamide (259, 90.88 mg, 160.11 μmol) in THF (5 mL) and Water (1 mL) was added Copper (II) sulfate (51.11 mg, 320.23 μmol, 14.20 uL) and (+)-Sodium L-ascorbate (63.44 mg, 320.23 μmol). The resulting mixture was stirred for 16 hr at rt. The resulting mixture was diluted with water and extracted with ethyl acetate (3×15 mL). The combined organic extracts were washed with water and brine solution, dried over anhydrous sodium sulfate, filtered and evaporated the solvent completely under reduced pressure. The resulting crude was purified by reverse phase prep HPLC to yield 4-anilino-N-cyclopropyl-6-[4-[4-[4-[[2-(2,6-dioxo-3-piperidyl)-1,3-dioxo-isoindolin-4-yl]oxymethyl]triazol-1-yl]butylcarbamoyl]-3-fluoro-phenyl]-7-methoxy-quinoline-3-carboxamide (Compound 414, 15 mg, 16.34 μmol, 10.21% yield) as a pale yellow solid. ¹H NMR (400 MHz, Methanol-d₄) δ 8.70 (s, 1H), 8.14 (s, 1H), 8.02 (s, 1H), 7.76 (dd, J=8.5, 7.3 Hz, 1H), 7.68 (t, J=7.9 Hz, 1H), 7.59 (d, J=8.5 Hz, 1H), 7.53-7.47 (m, 2H), 7.47-7.40 (m, 2H), 7.36-7.32 (m, 3H), 7.22 (s, 1H), 7.20 (d, J=5.7 Hz, 1H), 5.45 (s, 2H), 5.06 (dd, J=12.4, 5.4 Hz, 1H), 4.50 (t, J=7.0 Hz, 2H), 4.05 (s, 3H), 3.43 (t, J=6.8 Hz, 2H), 2.82 (ddd, J=17.7, 14.2, 5.0 Hz, 1H), 2.75-2.64 (m, 2H), 2.63-2.54 (m, 1H), 2.14-2.05 (m, 1H), 2.05-1.96 (m, 2H), 1.62 (p, J=6.9 Hz, 2H), 0.72 (td, J=7.2, 5.1 Hz, 2H), 0.53-0.46 (m, 2H). LCMS (ES+): m/z 880 [M+H]⁺

### Synthesis of Compound 415

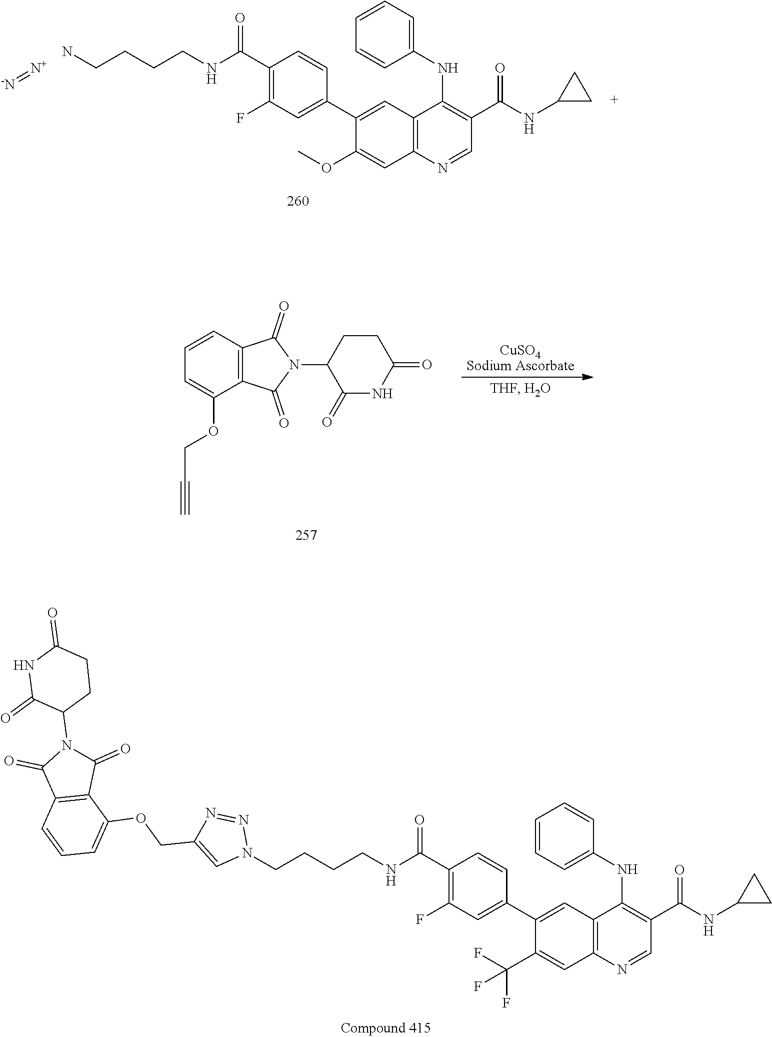

To a stirred solution of 4-anilino-6-[4-(4-azidobutylcarbamoyl)-3-fluoro-phenyl]-N-cyclopropyl-7-(trifluoromethyl)quinoline-3-carboxamide (260, 80 mg, 132.10 μmol) and 2-(2,6-dioxo-3-piperidyl)-4-prop-2-ynoxy-isoindoline-1,3-dione (257, 61.88 mg, 198.16 μmol) in THF/H2O (4/1 mL) was added (+)-Sodium L-ascorbate (52.34 mg, 264.21 μmol) and Copper (II) sulfate (42.17 mg, 264.21 μmol, 11.71 uL). The reaction mixture was stirred for 16 hr at 25° C. The reaction mixture was evaporated under reduced pressure, the resulting crude was purified by reverse phase preparative HPLC to yield 4-anilino-N-cyclopropyl-6-[4-[4-[4-[[2-(2,6-dioxo-3-piperidyl)-1,3-dioxo-isoindolin-4-yl]oxymethyl]triazol-1-yl]butylcarbamoyl]-3-fluoro-phenyl]-7-(trifluoromethyl)quinoline-3-carboxamide (Compound 415, 8.98 mg, 9.62 μmol, 7.28% yield) as a light yellow solid. ¹H NMR (400 MHz, Methanol-d₄) δ 8.86 (s, 1H), 8.34 (s, 1H), 8.25 (s, 1H), 8.15 (s, 1H), 7.81-7.71 (m, 2H), 7.60 (d, J=8.4 Hz, 1H), 7.45 (dd, J=7.6, 6.0 Hz, 3H), 7.36 (t, J=7.4 Hz, 1H), 7.29 (d, J=7.7 Hz, 2H), 7.19 (dd, J=15.4, 9.5 Hz, 2H), 5.46 (s, 2H), 5.07 (dd, J=12.5, 5.5 Hz, 1H), 4.51 (t, J=7.0 Hz, 2H), 3.44 (t, J=6.7 Hz, 2H), 2.92-2.61 (m, 4H), 2.48 (s, 1H), 2.15-2.06 (m, 1H), 2.07-1.97 (m, 1H), 1.70-1.58 (m, 2H), 0.72-0.63 (m, 2H), 0.47-0.39 (m, 2H). LCMS (ES+): m/z 918 [M+H]⁺

### Synthesis of Compound 416

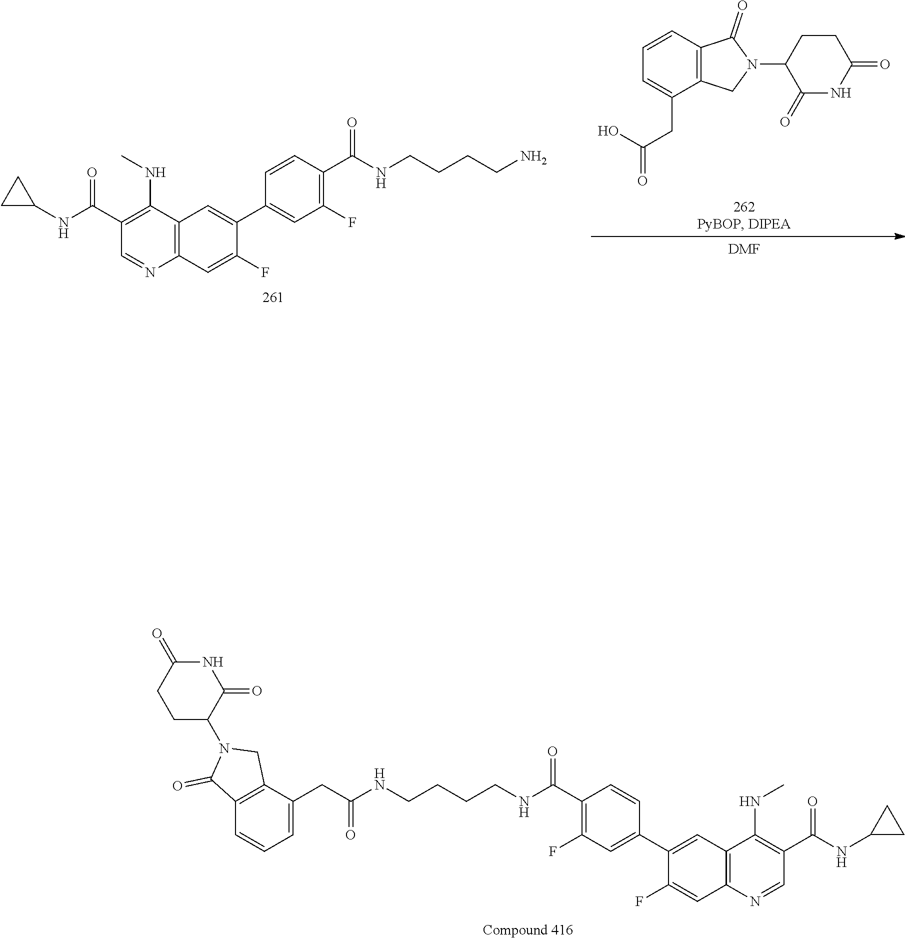

To a stirred solution of 6-[4-(4-aminobutylcarbamoyl)-3-fluoro-phenyl]-N-cyclopropyl-7-fluoro-4-(methylamino)quinoline-3-carboxamide (261, 70 mg, 149.73 μmol) and 2-[2-(2,6-dioxo-3-piperidyl)-1-oxo-isoindolin-4-yl]acetic acid (262, 67.89 mg, 224.59 μmol) in DMF (5 mL) was added PyBOP (116.88 mg, 224.59 μmol) and DIPEA (96.76 mg, 748.65 μmol, 130.40 uL). The reaction mixture was stirred for 16 hr at 25° C. The reaction mixture was evaporated the solvent completely under reduced pressure, the resulting crude was purified by reverse phase preparative HPLC to yield N-cyclopropyl-6-[4-[4-[[2-[2-(2,6-dioxo-3-piperidyl)-1-oxo-isoindolin-4-yl]acetyl]amino]butylcarbamoyl]-3-fluoro-phenyl]-7-fluoro-4-(methylamino)quinoline-3-carboxamide (Compound 416, 28 mg, 36.09 μmol, 24.10% yield) as a light yellow color solid. ¹H NMR (400 MHz, DMSO-d₆) δ 11.01 (s, 1H), 9.53 (s, 1H), 8.89 (d, J=4.1 Hz, 1H), 8.74 (d, J=7.4 Hz, 1H), 8.67 (s, 1H), 8.48-8.40 (m, 1H), 8.18 (t, J=5.6 Hz, 1H), 7.81-7.72 (m, 2H), 7.66 (d, J=11.3 Hz, 1H), 7.63-7.58 (m, 2H), 7.52-7.44 (m, 2H), 5.14 (dd, J=13.2, 5.1 Hz, 1H), 4.48 (d, J=17.3 Hz, 1H), 4.38 (d, J=17.2 Hz, 1H), 3.27 (q, J=6.4 Hz, 2H), 3.16-3.05 (m, 4H), 2.98-2.81 (m, 3H), 2.60 (d, J=17.5 Hz, 1H), 2.38 (dd, J=13.2, 4.5 Hz, 1H), 2.06-1.97 (m, 1H), 1.57-1.42 (m, 4H), 0.80-0.72 (m, 2H), 0.60 (p, J=4.6 Hz, 2H). LCMS (ES+): m/z 752 [M+H]⁺

### Synthesis of Compound 417

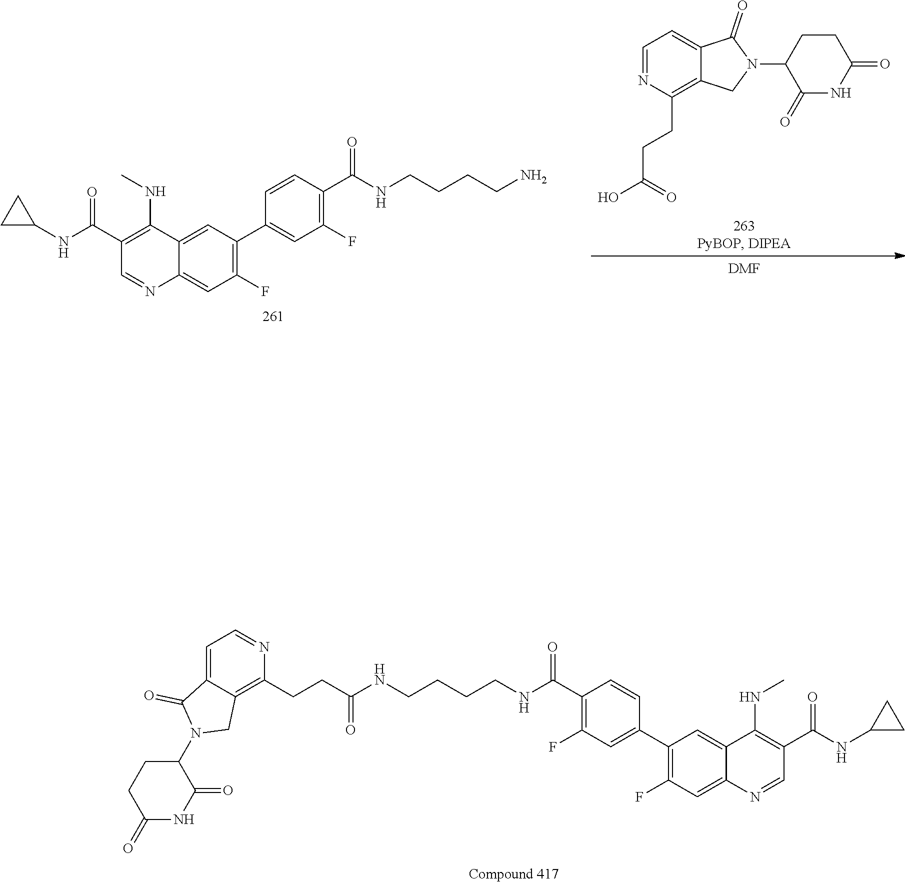

To a stirred solution of 6-[4-(4-aminobutylcarbamoyl)-3-fluoro-phenyl]-N-cyclopropyl-7-fluoro-4-(methylamino)quinoline-3-carboxamide (261, 50 mg, 106.95 μmol) and 3-[2-(2,6-dioxo-3-piperidyl)-1-oxo-3H-pyrrolo[3,4-c]pyridin-4-yl]propanoic acid (263, 50.90 mg, 160.42 μmol) in DMF (5 mL) was added PyBOP (83.48 mg, 160.42 μmol) and DIPEA (138.22 mg, 1.07 mmol, 186.28 uL). The reaction mixture was stirred for 16 hr at 25° C. The reaction mixture was evaporated the solvent completely under reduced pressure, the resulting crude was purified by reverse phase preparative HPLC to yield pure product N-cyclopropyl-6-[4-[4-[3-[2-(2,6-dioxo-3-piperidyl)-1-oxo-3H-pyrrolo[3,4-c]pyridin-4-yl]propanoylamino]butylcarbamoyl]-3-fluoro-phenyl]-7-fluoro-4-(methylamino)quinoline-3-carboxamide (Compound 417, 30 mg, 38.42 μmol, 35.93% yield) as a light yellow solid. ¹H NMR (400 MHz, DMSO-d₆) δ 11.04 (s, 1H), 9.56 (s, 1H), 8.90 (d, J=4.2 Hz, 1H), 8.75 (d, J=7.4 Hz, 1H), 8.67 (d, J=4.9 Hz, 2H), 8.45-8.36 (m, 1H), 7.90 (t, J=5.7 Hz, 1H), 7.84-7.72 (m, 2H), 7.66 (d, J=11.2 Hz, 1H), 7.61 (d, J=7.9 Hz, 1H), 7.57 (d, J=5.0 Hz, 1H), 5.17 (dd, J=13.2, 5.2 Hz, 1H), 4.61 (d, J=17.7 Hz, 1H), 4.46 (d, J=17.7 Hz, 1H), 3.24 (q, J=6.4 Hz, 2H), 3.17-2.98 (m, 6H), 2.98-2.80 (m, 2H), 2.65-2.57 (m, 3H), 2.44-2.37 (m, 1H), 2.09-1.97 (m, 1H), 1.52-1.35 (m, 4H), 0.81-0.71 (m, 2H), 0.63-0.56 (m, 2H). LCMS (ES+): m/z 766 [M+H]⁺

### Synthesis of Compound 418

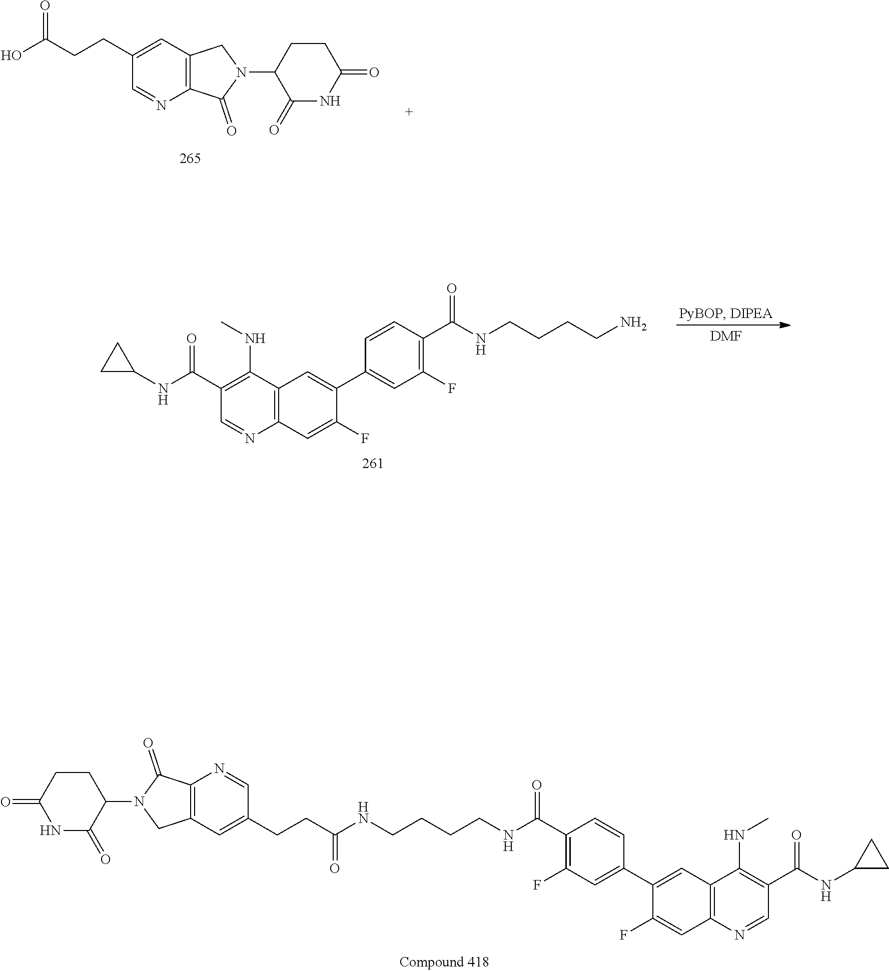

To a solution of 3-[6-(2,6-dioxo-3-piperidyl)-7-oxo-5H-pyrrolo[3,4-b]pyridin-3-yl]propanoic acid (265, 50 mg, 157.58 μmol) and 6-[4-(4-aminobutylcarbamoyl)-3-fluoro-phenyl]-N-cyclopropyl-7-fluoro-4-(methylamino)quinoline-3-carboxamide (261, 81.04 mg, 173.34 μmol) in DMF (5 mL) was added DIPEA (101.83 mg, 787.91 μmol, 137.24 uL) and PyBOP (123.01 mg, 236.37 μmol). The resulting mixture was stirred for 16 hr at 25° C. Added water (20 mL) to the reaction mixture and extracted with ethyl acetate (3×25 mL). The combined organic extracts were dried over anhydrous sodium sulfate, filtered and evaporated the solvent completely under reduced pressure. The resulting crude was purified by reverse phase prep HPLC to yield N-cyclopropyl-6-[4-[4-[3-[6-(2,6-dioxo-3-piperidyl)-7-oxo-5H-pyrrolo[3,4-b]pyridin-3-yl]propanoylamino]butylcarbamoyl]-3-fluoro-phenyl]-7-fluoro-4-(methylamino)quinoline-3-carboxamide (Compound 418, 1.66 mg, 2.12 μmol, 1.35% yield) as an off white solid. ¹H NMR (400 MHz, Methanol-d₄) δ 8.63 (d, J=1.8 Hz, 1H), 8.44 (d, J=8.0 Hz, 1H), 8.36 (s, 1H), 7.93 (d, J=1.8 Hz, 1H), 7.82 (t, J=7.8 Hz, 1H), 7.60 (dt, J=8.0, 1.7 Hz, 1H), 7.55 (d, J=11.9, 2.2 Hz, 2H), 5.16 (dd, J=13.3, 5.1 Hz, 1H), 4.60 (s, 2H), 4.54 (d, J=17.4 Hz, 1H), 4.47 (d, J=17.3 Hz, 1H), 3.12 (d, J=13.5 Hz, 4H), 2.96-2.83 (m, 2H), 2.77 (ddd, J=17.6, 4.7, 2.4 Hz, 1H), 2.49 (qd, J=13.2, 4.7 Hz, 2H), 2.17 (ddt, J=13.2, 5.7, 2.9 Hz, 1H), 1.56-1.47 (m, 4H), 0.84 (td, J=7.2, 5.1 Hz, 2H), 0.68-0.63 (m, 2H), 0.10 (d, J=2.2 Hz, 3H). LCMS (ES+): m/z 767 [M+H]⁺

### Synthesis of Compound 419

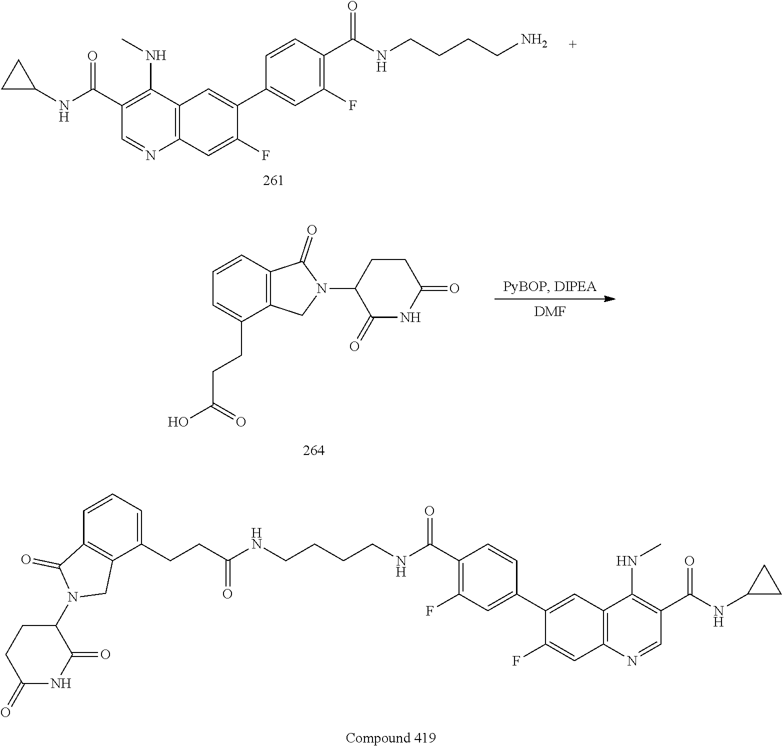

To a stirred solution of 6-[4-(4-aminobutylcarbamoyl)-3-fluoro-phenyl]-N-cyclopropyl-7-fluoro-4-(methylamino)quinoline-3-carboxamide (261, 50 mg, 106.95 μmol), 3-[2-(2,6-dioxo-3-piperidyl)-1-oxo-isoindolin-4-yl]propanoic acid (264, 50.74 mg, 160.42 μmol) in DMF (5 mL) was added PyBOP (83.48 mg, 160.42 μmol) and DIPEA (138.22 mg, 1.07 mmol, 186.28 uL). The reaction mixture was stirred for 16 hr at 25° C. The solvent was evaporated completely under reduced pressure. The resulting crude was purified by reverse phase preparative HPLC to yield N-cyclopropyl-6-[4-[4-[3-[2-(2,6-dioxo-3-piperidyl)-1-oxo-isoindolin-4-yl]propanoylamino]butylcarbamoyl]-3-fluoro-phenyl]-7-fluoro-4-(methylamino)quinoline-3-carboxamide (Compound 419, 8 mg, 10.20 μmol, 9.53% yield) as a light yellow color solid. ¹H NMR (400 MHz, Methanol-d₄) δ 8.67 (d, J=7.1 Hz, 2H), 8.56 (s, 1H), 7.86 (t, J=7.7 Hz, 1H), 7.67 (d, J=10.4 Hz, 1H), 7.61 (q, J=11.9, 10.1 Hz, 3H), 7.52-7.44 (m, 2H), 5.15 (dd, J=13.3, 5.2 Hz, 1H), 4.57 (d, J=17.0 Hz, 1H), 4.49 (d, J=17.0 Hz, 1H), 3.39-3.34 (m, 2H), 3.20-3.13 (m, 2H), 3.02 (t, J=7.3 Hz, 2H), 2.92 (dt, J=8.7, 4.8 Hz, 1H), 2.83-2.74 (m, 1H), 2.59-2.46 (m, 3H), 2.23-2.13 (m, 1H), 1.53-1.42 (m, 3H), 0.91-0.83 (m, 2H), 0.73-0.64 (m, 2H). LCMS (ES+): m/z 766 [M+H]⁺

### Synthesis of Compound 420

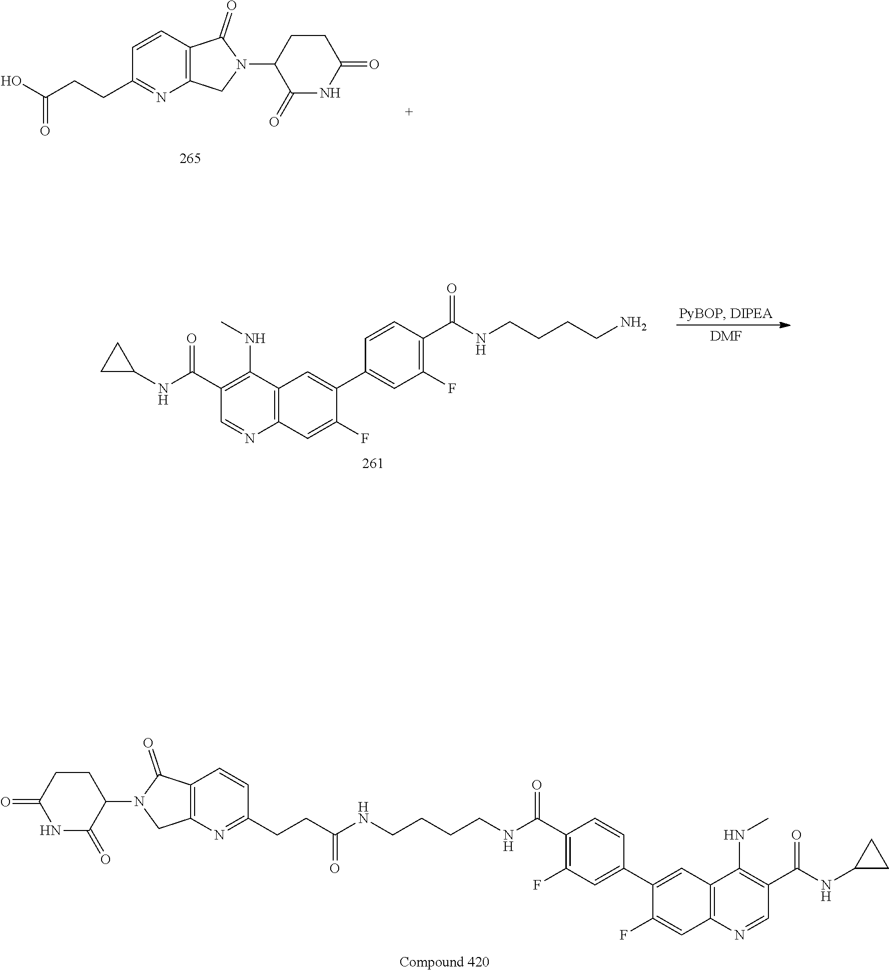

To a solution of 3-[6-(2,6-dioxo-3-piperidyl)-5-oxo-7H-pyrrolo[3,4-b]pyridin-2-yl]propanoic acid (265, 50 mg, 157.58 μmol) and 6-[4-(4-aminobutylcarbamoyl)-3-fluoro-phenyl]-N-cyclopropyl-7-fluoro-4-(methylamino)quinoline-3-carboxamide (261, 88.41 mg, 189.10 μmol, 061) in DMF (5 mL) was added DIPEA (101.83 mg, 787.91 μmol, 137.24 uL) and PyBOP (123.01 mg, 236.37 μmol). The resulting mixture was stirred for 16 hr at 25° C. Added water (20 mL) to the reaction mixture and extracted with ethyl acetate (3×25 mL). The combined organic extracts were dried over anhydrous sodium sulfate, filtered and evaporated the solvent completely under reduced pressure. The resulting crude was purified by Reverse phase prep HPLC to yield N-cyclopropyl-6-[4-[4-[3-[6-(2,6-dioxo-3-piperidyl)-5-oxo-7H-pyrrolo[3,4-b]pyridin-2-yl]propanoylamino]butylcarbamoyl]-3-fluoro-phenyl]-7-fluoro-4-(methylamino)quinoline-3-carboxamide (Compound 420, 15 mg, 19.05 μmol, 12.09% yield) as an off white solid. ¹H NMR (400 MHz, Methanol-d₄) δ 8.52-8.47 (m, 2H), 8.41 (s, 1H), 8.08 (d, J=8.0 Hz, 1H), 7.87-7.80 (m, 1H), 7.63-7.53 (m, 3H), 7.45 (d, J=8.0 Hz, 1H), 5.17 (dd, J=13.3, 5.1 Hz, 1H), 4.52 (d, J=17.5 Hz, 1H), 4.46 (d, J=17.5 Hz, 1H), 3.47-3.38 (m, 2H), 3.22 (t, J=7.6 Hz, 3H), 3.19 (s, 3H), 2.91 (ddd, J=11.8, 7.8, 4.3 Hz, 1H), 2.77 (ddd, J=17.5, 4.7, 2.3 Hz, 1H), 2.69 (t, J=7.3 Hz, 2H), 2.50 (qd, J=13.2, 4.8 Hz, 1H), 2.18 (ddd, J=9.8, 5.2, 2.6 Hz, 1H), 1.99 (s, 1H), 1.94 (s, 1H), 1.71-1.51 (m, 4H), 0.84 (td, J=7.2, 5.1 Hz, 2H), 0.67 (td, J=4.4, 2.9 Hz, 2H). LCMS (ES+): m/z 767 [M+H]⁺

### Synthesis of Compound 421

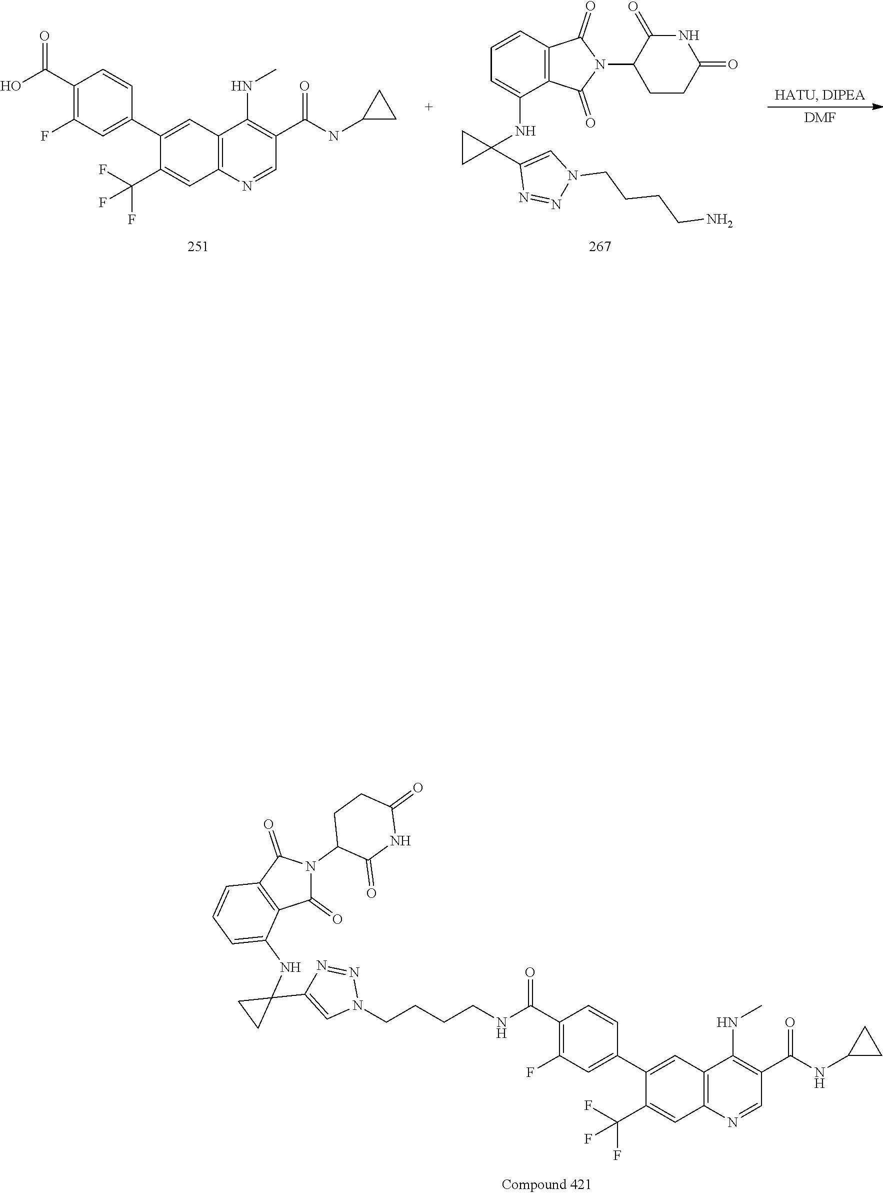

To a solution of 4-[3-(cyclopropylcarbamoyl)-4-(methylamino)-7-(trifluoromethyl)-6-quinolyl]-2-fluoro-benzoic acid (251, 60 mg, 134.11 μmol) and 4-[[1-[1-(4-aminobutyl)triazol-4-yl]cyclopropyl]amino]-2-(2,6-dioxo-3-piperidyl)isoindoline-1,3-dione (267, 66.60 mg, 147.53 μmol) in N,N-Dimethyl formamide (5 mL) was added DIPEA (86.67 mg, 670.57 μmol, 116.80 uL) followed by HATU (50.99 mg, 134.11 μmol) and then the reaction mixture was stirred at room temperature for 16 hours. Ice cold water (15 mL) was added to reaction mixture and stirred for 5 minutes, and then extracted with ethyl acetate. The combined organics were dried over anhydrous sodium sulfate and then concentrated to yield the crude product, which was purified by prep-HPLC(SUNFIRE OBD C18(100×30)MM 5μ) Mobile phase: A: 0.1% TFA in water B: ACN) to yield N-cyclopropyl-6-[4-[4-[4-[1-[[2-(2,6-dioxo-3-piperidyl)-1,3-dioxo-isoindolin-4-yl]amino]cyclopropyl]triazol-1-yl]butylcarbamoyl]-3-fluoro-phenyl]-4-(methylamino)-7-(trifluoromethyl)quinoline-3-carboxamide (Compound 421, 25 mg, 27.53 μmol, 20.53% yield) as yellow colored solid. ¹H NMR (400 MHz, DMSO-d₆) δ 11.10 (s, 1H), 9.53 (s, 1H), 8.90 (d, J=4.1 Hz, 1H), 8.79 (s, 1H), 8.56 (s, 1H), 8.48 (t, J=5.9 Hz, 1H), 8.34 (s, 1H), 7.94 (s, 1H), 7.71 (t, J=7.7 Hz, 1H), 7.55 (dd, J=8.5, 7.2 Hz, 1H), 7.39 (d, J=10.8 Hz, 1H), 7.32 (d, J=7.9 Hz, 1H), 7.27 (s, 1H), 7.12-7.04 (m, 2H), 5.07 (dd, J=12.9, 5.4 Hz, 1H), 4.30 (t, J=7.0 Hz, 2H), 3.26 (q, J=6.6 Hz, 2H), 3.09 (s, 3H), 2.95-2.80 (m, 2H), 2.62-2.53 (m, 2H), 2.07-1.98 (m, 1H), 1.82 (p, J=7.2 Hz, 2H), 1.50-1.38 (m, 4H), 1.30 (d, J=4.2 Hz, 2H), 0.75 (dt, J=6.9, 3.3 Hz, 2H), 0.63-0.56 (m, 2H). LCMS (ES+): m/z 881 [M+H]⁺

### Synthesis of Compound 422

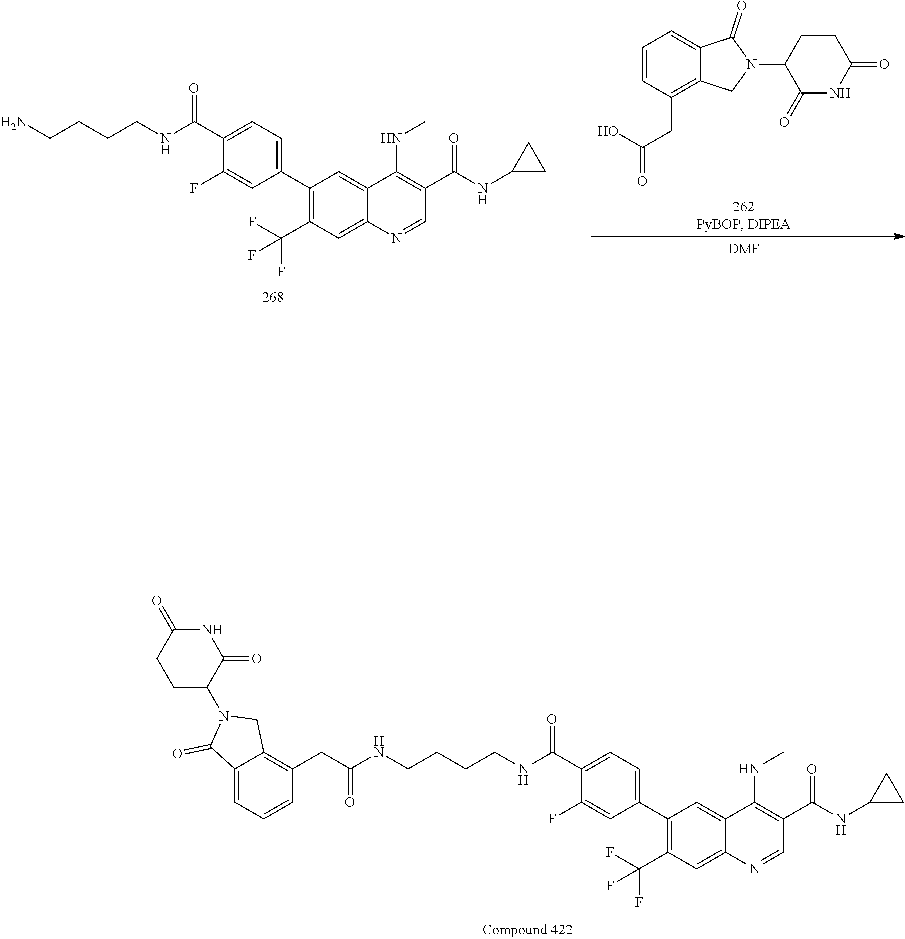

To a stirred solution of 6-[4-(4-aminobutylcarbamoyl)-3-fluoro-phenyl]-N-cyclopropyl-4-(methylamino)-7-(trifluoromethyl)quinoline-3-carboxamide (268, 70 mg, 135.26 μmol) and 2-[2-(2,6-dioxo-3-piperidyl)-1-oxo-isoindolin-4-yl]acetic acid (262, 44.98 mg, 148.79 μmol) in DMF (5 mL) was added DIPEA (87.41 mg, 676.31 μmol, 117.80 uL) and PyBOP (70.39 mg, 135.26 μmol). The reaction mixture was stirred for 16 hr at 25° C. The reaction mixture was diluted with water (10 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[4-[4-[[2-[2-(2,6-dioxo-3-piperidyl)-1-oxo-isoindolin-4-yl]acetyl]amino]butylcarbamoyl]-3-fluoro-phenyl]-4-(methylamino)-7-(trifluoromethyl)quinoline-3-carboxamide (Compound 422, 15 mg, 16.88 μmol, 12.48% yield) as white solid. ¹H NMR (400 MHz, DMSO-d₆) δ 11.00 (s, 1H), 8.61 (d, J=4.3 Hz, 1H), 8.46-8.41 (m, 2H), 8.33 (s, 1H), 8.21-8.14 (m, 2H), 7.87-7.80 (m, 1H), 7.68 (t, J=7.7 Hz, 1H), 7.60 (dd, J=7.2, 1.5 Hz, 1H), 7.52-7.43 (m, 2H), 7.37 (d, J=11.0 Hz, 1H), 7.32 (d, J=7.9 Hz, 1H), 5.14 (dd, J=13.3, 5.1 Hz, 1H), 4.48 (d, J=17.2 Hz, 1H), 4.38 (d, J=17.2 Hz, 1H), 3.53 (s, 2H), 3.30-3.21 (m, 2H), 3.09 (q, J=6.5 Hz, 2H), 2.93 (d, J=5.0 Hz, 3H), 2.89-2.79 (m, 1H), 2.64-2.56 (m, 1H), 2.38 (dd, J=13.1, 4.5 Hz, 1H), 2.06-1.98 (m, 1H), 1.58-1.41 (m, 4H), 0.70 (td, J=7.0, 4.8 Hz, 2H), 0.59-0.52 (m, 2H). LCMS (ES+): m/z 802 [M+H]⁺

### Synthesis of Compound 423

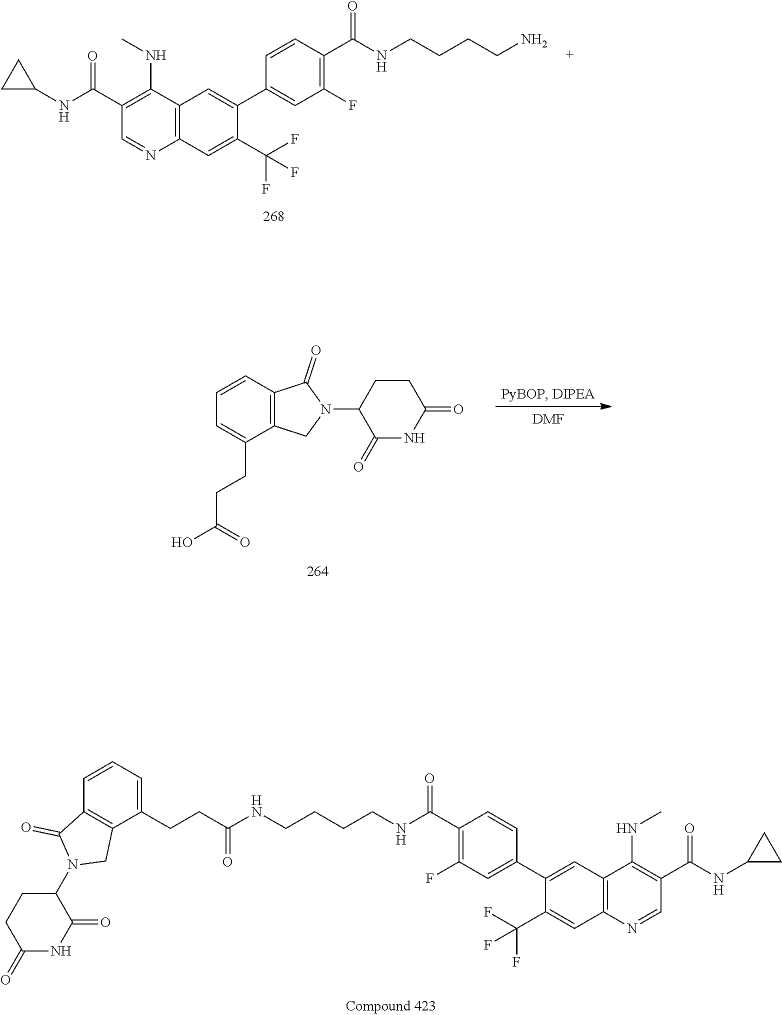

To a stirred solution of 6-[4-(4-aminobutylcarbamoyl)-3-fluoro-phenyl]-N-cyclopropyl-4-(methylamino)-7-(trifluoromethyl)quinoline-3-carboxamide (268, 50 mg, 96.62 μmol), 3-[2-(2,6-dioxo-3-piperidyl)-1-oxo-isoindolin-4-yl]propanoic acid (264, 45.84 mg, 144.92 μmol) in DMF (5 mL) was added PyBOP (75.42 mg, 144.92 μmol) and DIPEA (124.87 mg, 966.15 μmol, 168.28 uL). The reaction mixture was stirred for 16 hr at 25° C. The solvent was evaporated completely under reduced pressure, the resulting crude was purified by reverse phase preparative HPLC to yield N-cyclopropyl-6-[4-[4-[3-[2-(2,6-dioxo-3-piperidyl)-1-oxo-isoindolin-4-yl]propanoylamino]butylcarbamoyl]-3-fluoro-phenyl]-4-(methylamino)-7-(trifluoromethyl)quinoline-3-carboxamide (Compound 423, 5 mg, 5.94 μmol, 6.15% yield) as a light yellow color solid. ¹H NMR (400 MHz, Methanol-d₄) δ 8.68 (s, 1H), 8.49 (s, 1H), 8.26 (s, 1H), 7.81 (t, J=7.8 Hz, 1H), 7.64 (dd, J=7.0, 1.7 Hz, 1H), 7.52-7.44 (m, 2H), 7.36 (t, J=8.6 Hz, 2H), 5.16 (dd, J=13.3, 5.2 Hz, 1H), 4.57 (d, J=17.1 Hz, 1H), 4.49 (d, J=17.1 Hz, 1H), 3.40-3.33 (m, 2H), 3.23 (s, 2H), 3.19-3.13 (m, 2H), 3.02 (t, J=7.2 Hz, 2H), 2.98-2.85 (m, 2H), 2.83-2.73 (m, 1H), 2.61-2.44 (m, 3H), 2.23-2.13 (m, 2H), 1.54-1.40 (m, 4H), 0.87 (td, J=7.3, 5.2 Hz, 2H), 0.73-0.65 (m, 2H). LCMS (ES+): m/z 816 [M+H]⁺

### Synthesis of Compound 424

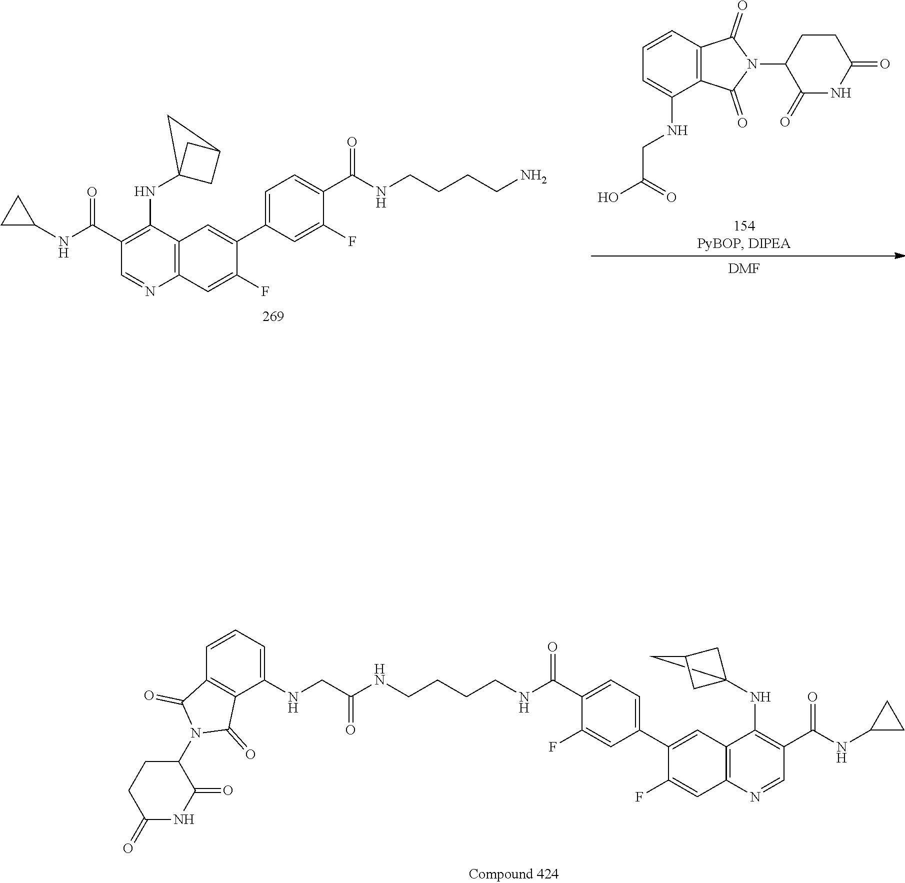

To a solution of 2-[[2-(2,6-dioxo-3-piperidyl)-1,3-dioxo-isoindolin-4-yl]amino]acetic acid (154, mg, 150.93 μmol) and 6-[4-(4-aminobutylcarbamoyl)-3-fluoro-phenyl]-4-(3-bicyclo[1.1.1]pentanylamino)-N-cyclopropyl-7-fluoro-quinoline-3-carboxamide (269, 78.42 mg, 150.93 μmol) in DMF (5 mL) was added DIPEA (97.53 mg, 754.65 μmol, 131.45 uL) and PyBOP (117.81 mg, 226.39 μmol). The resulting mixture was stirred for 16 hr at 25° C. Added water (20 mL) to the reaction mixture and extracted with ethyl acetate (3×25 mL). The combined organic extracts were dried over anhydrous sodium sulfate, filtered and evaporated the solvent completely under reduced pressure. The resulting crude was purified by reverse phase prep HPLC to yield 4-(3-bicyclo[1.1.1]pentanylamino)-N-cyclopropyl-6-[4-[4-[[2-[[2-(2,6-dioxo-3-piperidyl)-1,3-dioxo-isoindolin-4-yl]amino]acetyl]amino]butylcarbamoyl]-3-fluoro-phenyl]-7-fluoro-quinoline-3-carboxamide (Compound 424, 20 mg, 23.74 μmol, 15.73% yield) as an yellow solid. ¹H NMR (400 MHz, Methanol-d₄) δ 8.76 (s, 1H), 8.48 (d, J=8.2 Hz, 1H), 7.84 (t, J=7.8 Hz, 1H), 7.66 (d, J=11.7 Hz, 1H), 7.60-7.50 (m, 3H), 7.09 (d, J=7.2 Hz, 1H), 6.89 (d, J=8.5 Hz, 1H), 5.06 (dd, J=12.4, 5.4 Hz, 1H), 4.01 (s, 2H), 3.45-3.38 (m, 2H), 2.95-2.79 (m, 1H), 2.77-2.65 (m, 2H), 2.41 (s, 1H), 2.13-2.01 (m, 7H), 1.62 (s, 4H), 0.88-0.81 (m, 2H), 0.73-0.64 (m, 2H). LCMS (ES+): m/z 833 [M+H]⁺

### Synthesis of Compound 425

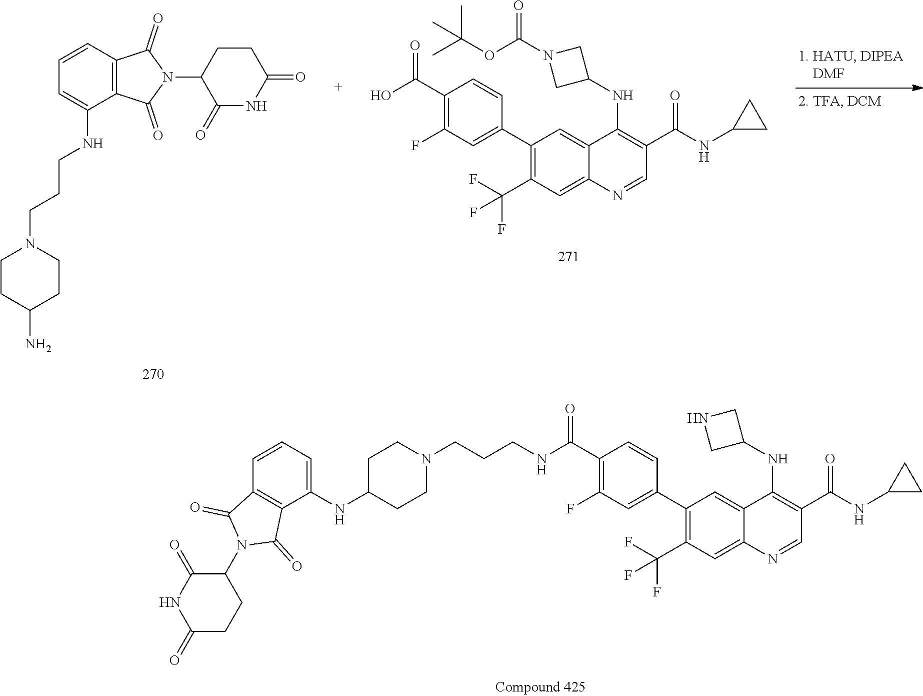 [SMILES: NC1CCN(CCCNc2cccc3c2C(=O)N(C2CCC(=O)NC2=O)C3=O)CC1 ; CC(C)(C)OC(=O)N1CC(Nc2c(C(=O)NC3CC3)cnc3cc(C(F)(F)F)c(-c4ccc(C(=O)O)c(F)c4)cc23)C1 ; O=C1CCC(N2C(=O)c3cccc(NC4CCN(CCCNC(=O)c5ccc(-c6cc7c(NC8CNC8)c(C(=O)NC8CC8)cnc7cc6C(F)(F)F)cc5F)CC4)c3C2=O)C(=O)N1]

Into a 25 mL single-necked round-bottomed flask containing a well-stirred solution of 4-[3-(4-amino-1-piperidyl)propylamino]-2-(2,6-dioxo-3-piperidyl)isoindoline-1,3-dione (270, 25 mg, 60.46 μmol) and 4-[4-[(1-tert-butoxycarbonylazetidin-3-yl)amino]-3-(cyclopropylcarbamoyl)-7-(trifluoromethyl)-6-quinolyl]-2-fluoro-benzoic acid (271, 35.59 mg, 60.46 μmol) in anhydrous DMF (3 mL) were added DIPEA (23.44 mg, 181.39 μmol, 31.59 uL) and HATU (34.49 mg, 90.70 μmol) under nitrogen atmosphere at room temperature. The resulting mixture was stirred at room temperature for 16 h. To the crude mass 20 mL of water was added, aqueous phase was extracted twice with EtOAc (2×25 mL). The organic layer was dried (anhydrous Na₂SO₄), filtered and the filtrate was concentrated under reduced pressure to afford crude residue, which was dissolved in DCM (4 mL). To this mixture was added TFA (34.47 mg, 302.32 μmol, 23.29 uL) under nitrogen atmosphere at room temperature, the resulting mixture was stirred at room temperature for 30 minutes. The reaction mixture was concentrated under reduced pressure and purified by prep-HPLC (SUNFIRE OBD C18(100×30)MM 5μ) Mobile phase: A: 0.1% TFA in water B: ACN to afford 4-(azetidin-3-ylamino)-N-cyclopropyl-6-[4-[3-[4-[[2-(2,6-dioxo-3-piperidyl)-1,3-dioxo-isoindolin-4-yl]amino]-1-piperidyl]propylcarbamoyl]-3-fluoro-phenyl]-7-(trifluoromethyl)quinoline-3-carboxamide (Compound 425, 17 mg, 19.00 μmol, 31.43% yield) as a yellow solid. ¹H NMR (400 MHz, DMSO-d₆) δ 11.11 (s, 1H), 9.24 (s, 1H), 8.87 (d, J=4.0 Hz, 1H), 8.62 (s, 3H), 8.44 (s, 1H), 8.31 (s, 1H), 8.12 (s, 1H), 7.78 (t, J=7.7 Hz, 1H), 7.64 (dd, J=8.6, 7.1 Hz, 1H), 7.43 (d, J=11.0 Hz, 1H), 7.37 (d, J=8.0 Hz, 1H), 7.24 (d, J=8.7 Hz, 1H), 7.12 (d, J=7.0 Hz, 1H), 6.30 (d, J=8.1 Hz, 1H), 5.07 (dd, J=12.8, 5.5 Hz, 1H), 4.53 (q, J=7.4 Hz, 1H), 4.26-4.06 (m, 5H), 3.22-3.05 (m, 4H), 2.95-2.82 (m, 3H), 2.64-2.54 (m, 2H), 2.21 (d, J=13.2 Hz, 2H), 2.08-1.91 (m, 4H), 1.80-1.65 (m, 2H), 0.80-0.72 (m, 2H), 0.70-0.63 (m, 2H). LCMS (ES+): m/z 884 [M+H]⁺

### Synthesis of Compound 426

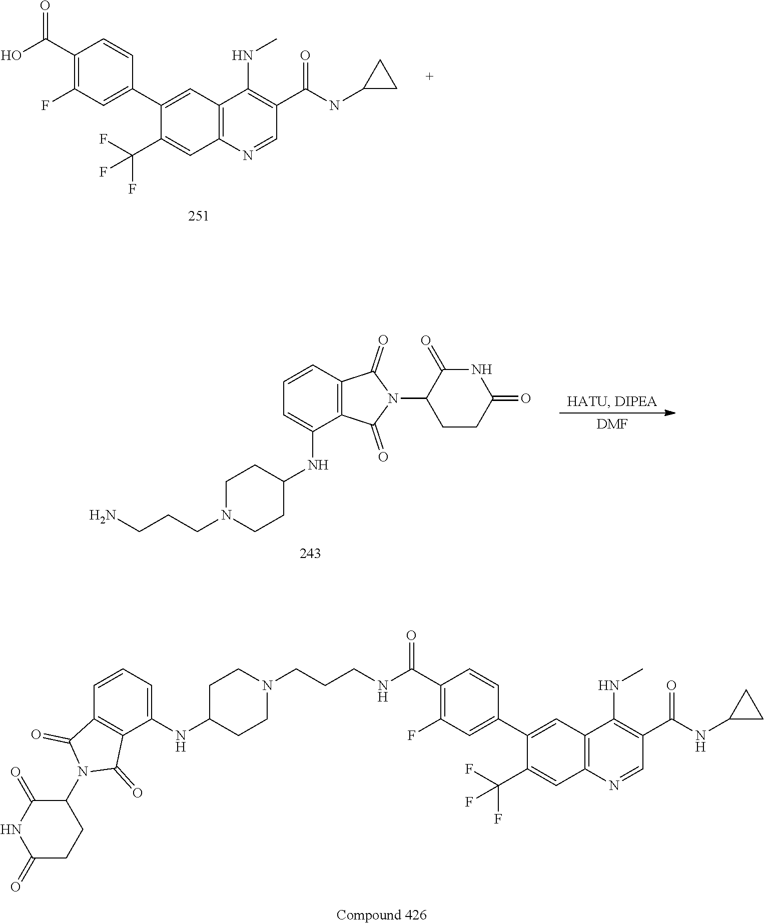

To a solution of 4-[3-(cyclopropylcarbamoyl)-4-(methylamino)-7-(trifluoromethyl)-6-quinolyl]-2-fluoro-benzoic acid (251, 50 mg, 111.76 μmol) and 4-[[1-(3-aminopropyl)-4-piperidyl]amino]-2-(2,6-dioxo-3-piperidyl)isoindoline-1,3-dione (243, 50.83 mg, 122.94 μmol) in DMF (5 mL) was added DIPEA (72.22 mg, 558.81 μmol, 97.33 uL) followed by HATU (63.74 mg, 167.64 μmol). The reaction mixture stirred at room temperature for 16 hours. Ice cold water (15 mL) was added to reaction mixture and stirred for 5 minutes, and then extracted with ethyl acetate. The combined organics were dried over anhydrous sodium sulfate and then concentrated to yield the crude product, which was purified by prep-HPLC(SUNFIRE OBD C18(100×30)MM 5μ) Mobile phase: A: 0.1% TFA in water B: ACN) to yield N-cyclopropyl-6-[4-[3-[4-[[2-(2,6-dioxo-3-piperidyl)-1,3-dioxo-isoindolin-4-yl]amino]-1-piperidyl]propylcarbamoyl]-3-fluoro-phenyl]-4-(methylamino)-7-(trifluoromethyl)quinoline-3-carboxamide (Compound 426, 18 mg, 20.83 μmol, 18.64% yield) as light yellow colored solid. ¹H NMR (400 MHz, Methanol-d₄) δ 8.68 (s, 1H), 8.48 (s, 1H), 8.28 (s, 1H), 7.91 (t, J=7.8 Hz, 1H), 7.61 (t, J=7.8 Hz, 1H), 7.43-7.33 (m, 2H), 7.16 (dd, J=19.2, 7.9 Hz, 2H), 5.07 (dd, J=12.5, 5.5 Hz, 1H), 3.99-3.88 (m, 1H), 3.74 (d, J=12.5 Hz, 2H), 3.57 (t, J=6.5 Hz, 2H), 3.27 (s, 3H), 3.22-3.11 (m, 2H), 2.99-2.89 (m, 2H), 2.89-2.80 (m, 1H), 2.78-2.64 (m, 2H), 2.39 (d, J=14.1 Hz, 2H), 2.18-2.06 (m, 3H), 1.83 (q, J=12.8, 12.4 Hz, 2H), 0.87 (td, J=7.2, 5.1 Hz, 2H), 0.71-0.66 (m, 2H). LCMS (ES+): m/z 843 [M+H]⁺

### Synthesis of Compound 427

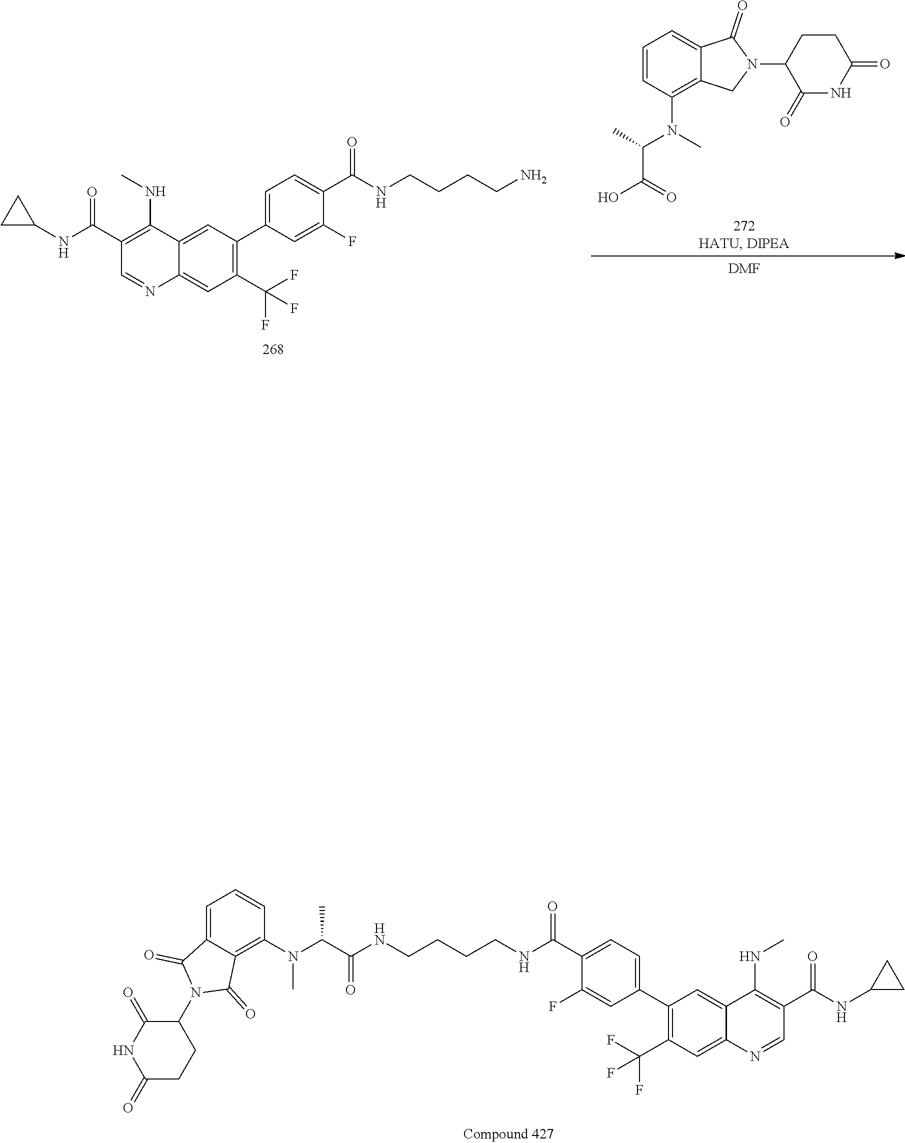

To a stirred solution of 6-[4-(4-aminobutylcarbamoyl)-3-fluoro-phenyl]-N-cyclopropyl-4-(methylamino)-7-(trifluoromethyl)quinoline-3-carboxamide (268, 50 mg, 96.62 μmol) and (2S)-2-[[2-(2,6-dioxo-3-piperidyl)-1,3-dioxo-isoindolin-4-yl]-methyl-amino]propanoic acid (272, 38.19 mg, 106.28 μmol) in DMF (5 mL) was added DIPEA (62.43 mg, 483.08 μmol, 84.14 uL) and HATU (55.10 mg, 144.92 μmol). The reaction mixture was stirred for 16 hr at 25° C. The reaction mixture was diluted with water (10 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[4-[4-[[(2S)-2-[[2-(2,6-dioxo-3-piperidyl)-1,3-dioxo-isoindolin-4-yl]-methyl-amino]propanoyl]amino]butylcarbamoyl]-3-fluoro-phenyl]-4-(methylamino)-7-(trifluoromethyl)quinoline-3-carboxamide (Compound 427, 5 mg, 5.55 μmol, 5.75% yield) as a light yellow solid. ¹H NMR (400 MHz, Methanol-d₄) δ 8.67 (s, 1H), 8.47 (s, 1H), 8.26 (s, 1H), 8.17 (s, 1H), 7.79 (dt, J=7.5, 4.3 Hz, 1H), 7.73-7.63 (m, 1H), 7.42-7.37 (m, 2H), 7.34 (t, J=8.7 Hz, 2H), 5.13 (dd, J=12.4, 5.4 Hz, 1H), 4.69-4.60 (m, 1H), 3.47-3.39 (m, 2H), 2.98-2.90 (m, 2H), 2.88 (s, 3H), 2.84-2.80 (m, 1H), 2.78-2.64 (m, 3H), 2.17-2.05 (m, 1H), 1.68 (s, 4H), 1.33 (dd, J=7.0, 1.9 Hz, 3H), 0.87 (td, J=7.2, 5.2 Hz, 2H), 0.73-0.65 (m, 2H). LCMS (ES+): m/z 859 [M+H]⁺

### Synthesis of Compound 428

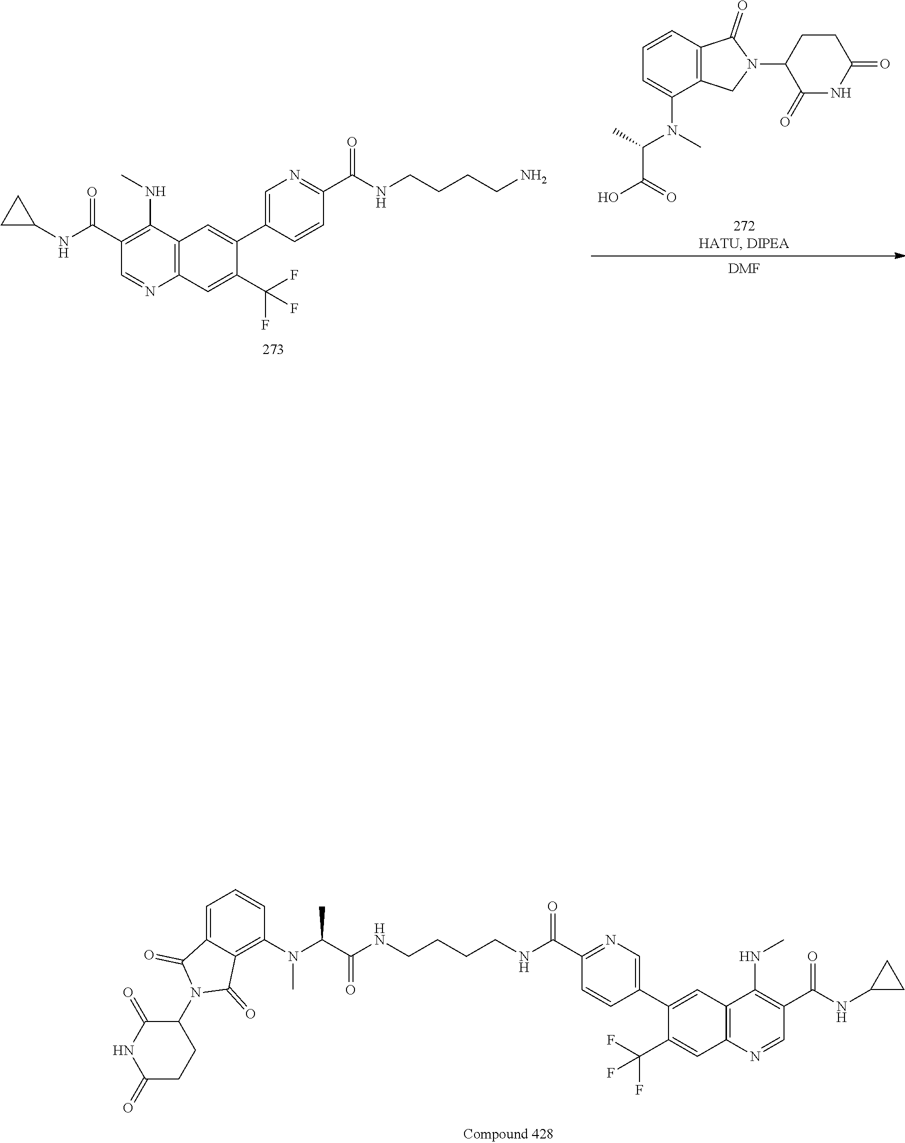

To a stirred solution of 6-[6-(4-aminobutylcarbamoyl)-3-pyridyl]-N-cyclopropyl-4-(methylamino)-7-(trifluoromethyl)quinoline-3-carboxamide (273, 37.99 mg, 75.90 μmol), (2S)-2-[[2-(2,6-dioxo-3-piperidyl)-1,3-dioxo-isoindolin-4-yl]-methyl-amino]propanoic acid (272, 30 mg, 83.49 μmol) in DMF (2.0 mL) was added DIPEA (49.05 mg, 379.49 μmol, 66.10 uL) and HATU (43.29 mg, 113.85 μmol). The resulting mixture was stirred for 16 hr at 25° C. The resulting mixture was diluted with water (10 mL) and extracted with ethyl acetate (3×15 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[6-[4-[[(2R)-2-[[2-(2,6-dioxo-3-piperidyl)-1,3-dioxo-isoindolin-4-yl]-methyl-amino]propanoyl]amino]butylcarbamoyl]-3-pyridyl]-4-(methylamino)-7-(trifluoromethyl)quinoline-3-carboxamide (Compound 428, 10 mg, 10.81 μmol, 14.24% yield) as an yellow solid. LCMS (ES+): m/z 842 [M+H]⁺

### Synthesis of Compound 429

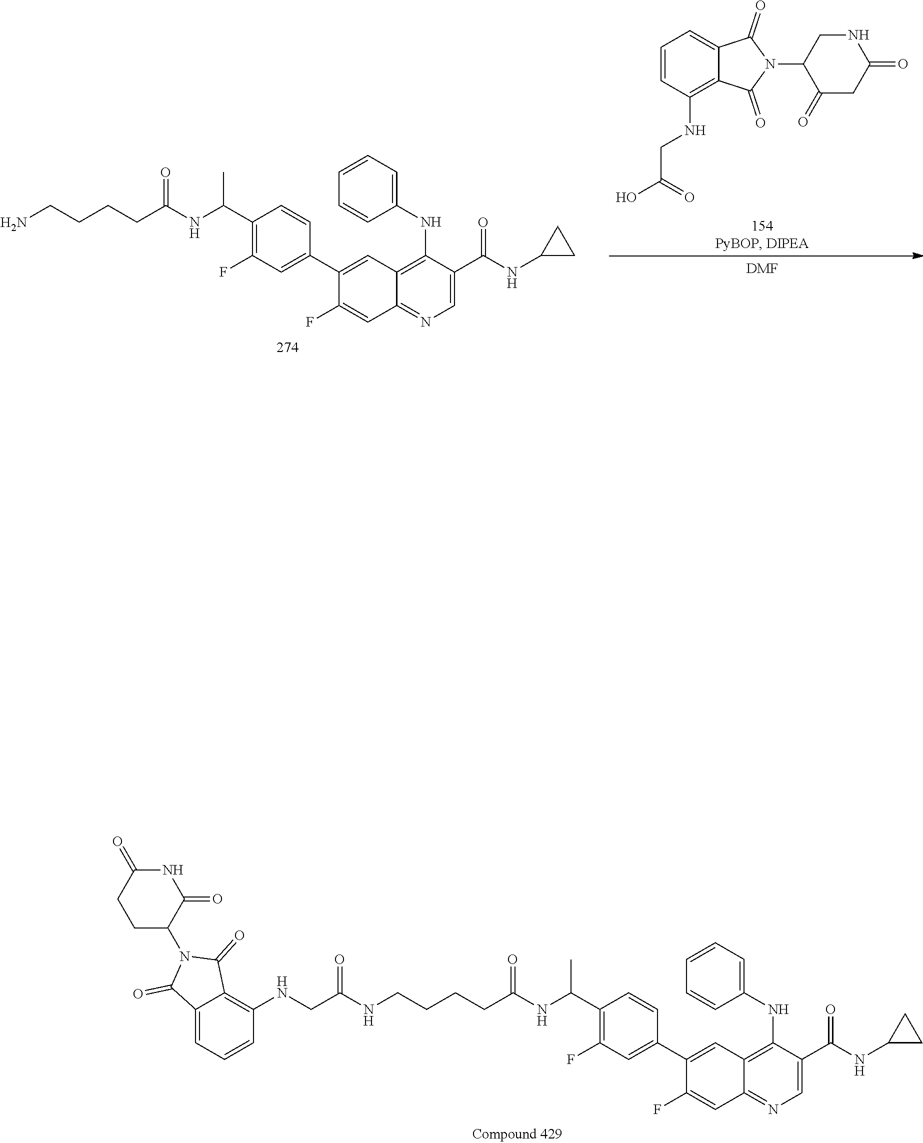

To a stirred solution of 2-[[2-(2,6-dioxo-3-piperidyl)-1,3-dioxo-isoindolin-4-yl]amino]acetic acid (154, 41.59 mg, 125.53 μmol) and 6-[4-[1-(5-aminopentanoylamino)ethyl]-3-fluoro-phenyl]-4-anilino-N-cyclopropyl-7-fluoro-quinoline-3-carboxamide (274, 70 mg, 125.53 μmol) in N,N-Dimethylformamide (3 mL) was added N,N-Diisopropylethylamine (48.67 mg, 376.59 μmol, 65.59 uL) followed by PyBOP (97.99 mg, 188.30 μmol). The reaction mixture was stirred at room temperature for 16 hours. Water was added to the reaction mixture and then extracted with ethyl acetate. The combined organics were dried over anhydrous sodium sulfate and then concentrated to yield the crude, which was purified by prep HPLC to yield 4-anilino-N-cyclopropyl-6-[4-[1-[5-[[2-[[2-(2,6-dioxo-3-piperidyl)-1,3-dioxo-isoindolin-4-yl]amino]acetyl]amino]pentanoylamino]ethyl]-3-fluoro-phenyl]-7-fluoro-quinoline-3-carboxamide (Compound 429, 10 mg, 10.87 μmol, 8.66% yield) as light yellow colored solid. ¹H NMR (400 MHz, Methanol-d₄) δ 8.70 (d, J=2.1 Hz, 1H), 8.16 (dd, J=8.3, 4.7 Hz, 1H), 7.61 (d, J=11.8 Hz, 1H), 7.51-7.44 (m, 1H), 7.39-7.29 (m, 3H), 7.22-7.13 (m, 2H), 7.13-7.05 (m, 3H), 7.02 (dd, J=7.1, 3.2 Hz, 1H), 6.75 (dd, J=8.6, 4.9 Hz, 1H), 5.22 (dd, J=7.0 Hz, 1H), 3.92-3.84 (m, 2H), 3.22-3.12 (m, 1H), 2.89-2.50 (m, 4H), 2.29-2.18 (m, 2H), 2.10-1.98 (m, 1H), 1.58 (dt, J=13.8, 7.2 Hz, 3H), 1.46 (p, J=7.3 Hz, 6H), 1.35-1.24 (m, 1H), 0.72-0.61 (m, 2H), 0.39 (pd, J=5.0, 3.3, 2.5 Hz, 2H). LCMS (ES+): m/z 871 [M+H]⁺

### Synthesis of Compound 430

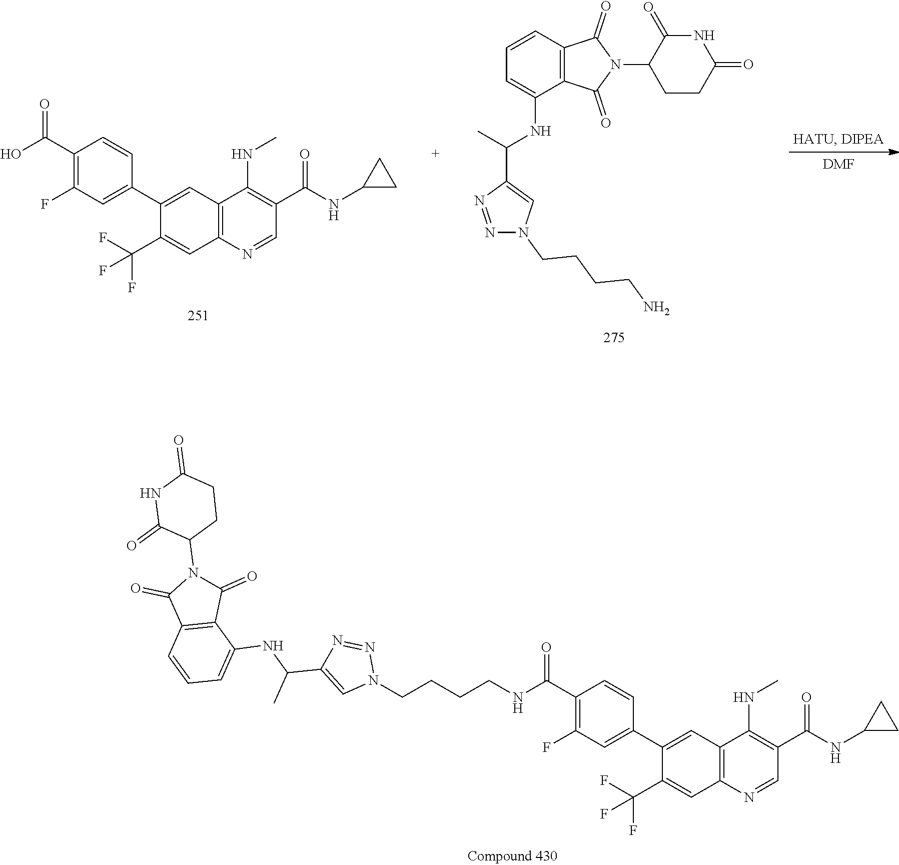

To a stirred solution of 4-[3-(cyclopropylcarbamoyl)-4-(methylamino)-7-(trifluoromethyl)-6-quinolyl]-2-fluoro-benzoic acid (251, 40 mg, 89.41 μmol) and 4-[1-[1-(4-aminobutyl)triazol-4-yl]ethylamino]-2-(2,6-dioxo-3-piperidyl)isoindoline-1,3-dione (275, 43.22 mg, 98.35 μmol) in DMF (5.0 mL) was added DIPEA (57.78 mg, 447.05 μmol, 77.87 uL) and HATU (50.99 mg, 134.11 μmol). The resulting mixture was stirred for 16 hr at 25° C. The resulting mixture was diluted with water (10 mL) and extracted with ethyl acetate (3×15 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[4-[4-[4-[1-[[2-(2,6-dioxo-3-piperidyl)-1,3-dioxo-isoindolin-4-yl]amino]ethyl]triazol-1-yl]butylcarbamoyl]-3-fluoro-phenyl]-4-(methylamino)-7-(trifluoromethyl)quinoline-3-carboxamide (Compound 430, 20 mg, 21.57 μmol, 24.13% yield) as an yellow solid. ¹H NMR (400 MHz, DMSO-d₆) δ 11.11 (s, 1H), 9.45 (s, 1H), 8.91 (d, J=4.2 Hz, 1H), 8.77 (s, 1H), 8.55 (s, 1H), 8.53-8.50 (m, 1H), 8.34 (s, 1H), 8.10 (s, 1H), 7.72 (t, J=7.7 Hz, 1H), 7.57 (t, J=7.8 Hz, 1H), 7.40 (d, J=10.8 Hz, 1H), 7.33 (d, J=8.0 Hz, 1H), 7.17 (dd, J=8.7, 4.4 Hz, 1H), 7.06 (d, J=7.1 Hz, 1H), 6.68 (d, J=7.7 Hz, 1H), 5.12-5.01 (m, 2H), 4.38 (td, J=7.2, 2.4 Hz, 2H), 3.29 (q, J=6.7 Hz, 2H), 3.08 (s, 3H), 2.90-2.81 (m, 2H), 2.61-2.54 (m, 2H), 2.08-1.96 (m, 1H), 1.87 (p, J=7.7 Hz, 2H), 1.58 (d, J=6.6 Hz, 3H), 1.53-1.43 (m, 2H), 0.79-0.71 (m, 2H), 0.59 (p, J=4.6 Hz, 2H). LCMS (ES+): m/z 869 [M+H]⁺

### Synthesis of Compound 431

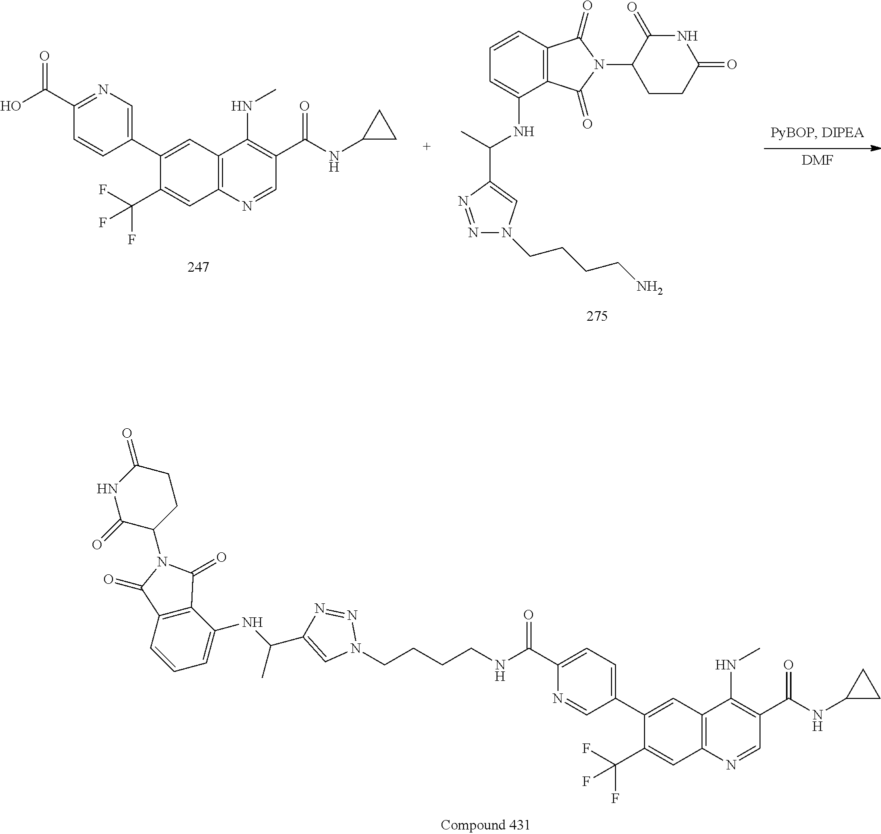

To a stirred solution of 5-[3-(cyclopropylcarbamoyl)-4-(methylamino)-7-(trifluoromethyl)-6-quinolyl]pyridine-2-carboxylic acid (247, 40 mg, 92.94 μmol) and 4-[1-[1-(4-aminobutyl)triazol-4-yl]ethylamino]-2-(2,6-dioxo-3-piperidyl)isoindoline-1,3-dione (275, 40.84 mg, 92.94 μmol) in DMF (3 mL) was added DIPEA (12.01 mg, 92.94 μmol, 16.19 uL) and PyBOP (58.04 mg, 111.53 μmol). The resulting mixture was stirred for 16 hr at 25° C. The resulting mixture was diluted with water (15 mL) and extracted with ethyl acetate (3×15 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[6-[4-[4-[1-[[2-(2,6-dioxo-3-piperidyl)-1,3-dioxo-isoindolin-4-yl]amino]ethyl]triazol-1-yl]butylcarbamoyl]-3-pyridyl]-4-(methylamino)-7-(trifluoromethyl)quinoline-3-carboxamide (Compound 431, 20 mg, 22.18 μmol, 23.87% yield) as an yellow solid. ¹H NMR (400 MHz, DMSO-d₆) δ 11.11 (s, 1H), 9.54 (s, 1H), 9.01 (t, J=6.1 Hz, 1H), 8.93 (d, J=4.1 Hz, 1H), 8.81 (s, 1H), 8.69 (d, J=2.1 Hz, 1H), 8.63 (s, 1H), 8.39 (s, 1H), 8.19 (d, J=8.0 Hz, 1H), 8.12-8.06 (m, 2H), 7.56 (t, J=7.9 Hz, 1H), 7.16 (dd, J=8.7, 4.1 Hz, 1H), 7.05 (d, J=7.2 Hz, 1H), 6.67 (d, J=7.7 Hz, 1H), 5.06 (dd, J=12.7, 5.7 Hz, 2H), 4.37 (td, J=7.1, 2.6 Hz, 2H), 3.39-3.27 (m, 2H), 3.10 (s, 3H), 2.91-2.81 (m, 2H), 2.61-2.54 (m, 2H), 2.08-1.97 (m, 1H), 1.88-1.76 (m, 2H), 1.57 (d, J=6.6 Hz, 3H), 1.51 (t, J=7.7 Hz, 2H), 0.80-0.72 (m, 2H), 0.64-0.56 (m, 2H). LCMS (ES+): m/z 852 [M+H]⁺

### Synthesis of Compound 432

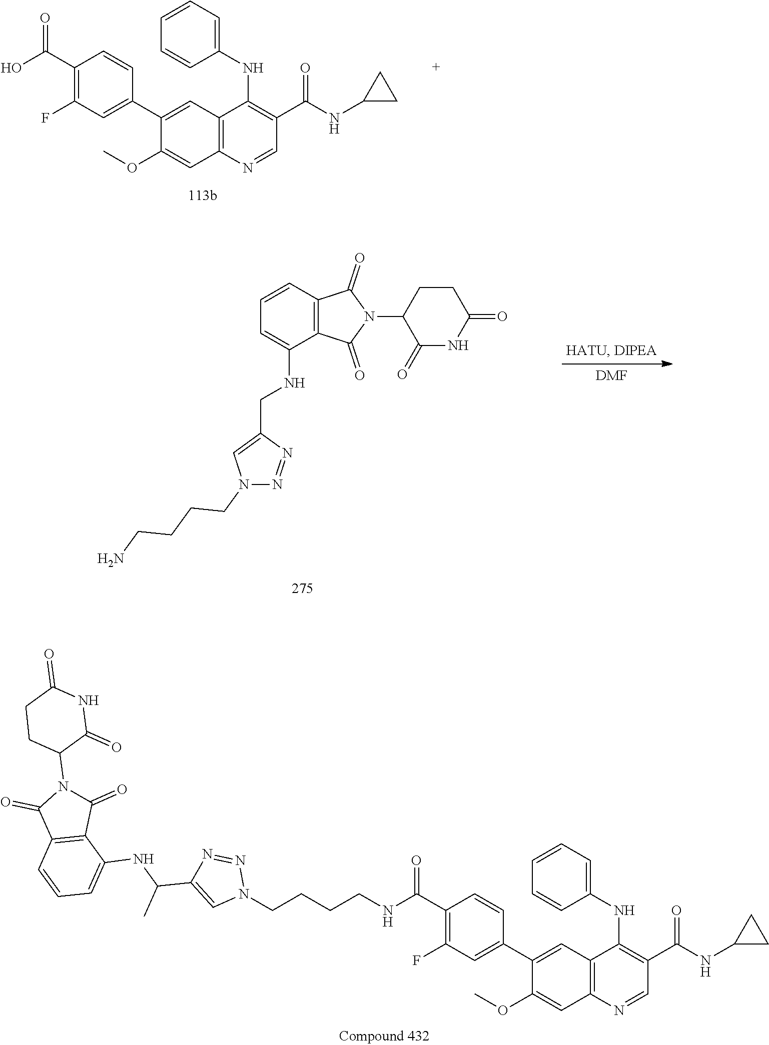

To a solution of 4-[4-anilino-3-(cyclopropylcarbamoyl)-7-methoxy-6-quinolyl]-2-fluoro-benzoic acid (113b, 50 mg, 106.05 μmol) and 4-[1-[1-(4-aminobutyl)triazol-4-yl]ethylamino]-2-(2,6-dioxo-3-piperidyl)isoindoline-1,3-dione (275, 46.61 mg, 106.05 μmol) in DMF (10 mL) was added DIPEA (68.53 mg, 530.25 μmol, 92.36 uL) and HATU (60.48 mg, 159.07 μmol). The resulting mixture was stirred for 16 hr at rt. Added water (20 mL) to the reaction mixture and extracted with ethyl acetate (3×25 mL). The combined organic extracts were dried over anhydrous sodium sulfate, filtered and evaporated the solvent completely under reduced pressure. The resulting crude was purified by Reverse phase prep HPLC to yield 4-anilino-N-cyclopropyl-6-[4-[4-[4-[1-[[2-(2,6-dioxo-3-piperidyl)-1,3-dioxo-isoindolin-4-yl]amino]ethyl]triazol-1-yl]butylcarbamoyl]-3-fluoro-phenyl]-7-methoxy-quinoline-3-carboxamide (Compound 432, 40 mg, 44.37 μmol, 41.84% yield) as an yellow solid. ¹H NMR (400 MHz, Methanol-d₄) δ 8.71 (d, J=1.5 Hz, 1H), 8.33 (s, 1H), 8.05 (s, 1H), 7.93 (s, 1H), 7.66 (t, J=7.9 Hz, 1H), 7.55-7.47 (m, 3H), 7.46-7.39 (m, 2H), 7.37-7.32 (m, 3H), 7.26-7.18 (m, 2H), 7.03-7.00 (m, 1H), 6.99 (d, J=2.8 Hz, 1H), 5.06-4.99 (m, 2H), 4.44 (t, J=7.0 Hz, 2H), 4.05 (d, J=1.4 Hz, 3H), 3.40 (t, J=6.7 Hz, 2H), 2.82 (ddd, J=17.4, 14.1, 5.1 Hz, 2H), 2.76-2.64 (m, 2H), 2.58 (s, 1H), 2.12-2.02 (m, 1H), 1.97 (p, J=7.1 Hz, 2H), 1.67 (d, J=6.7 Hz, 3H), 1.57 (p, J=7.1 Hz, 2H), 0.76-0.68 (m, 2H), 0.54-0.45 (m, 2H). LCMS (ES+): m/z 893 [M+H]⁺

### Synthesis of Compound 433

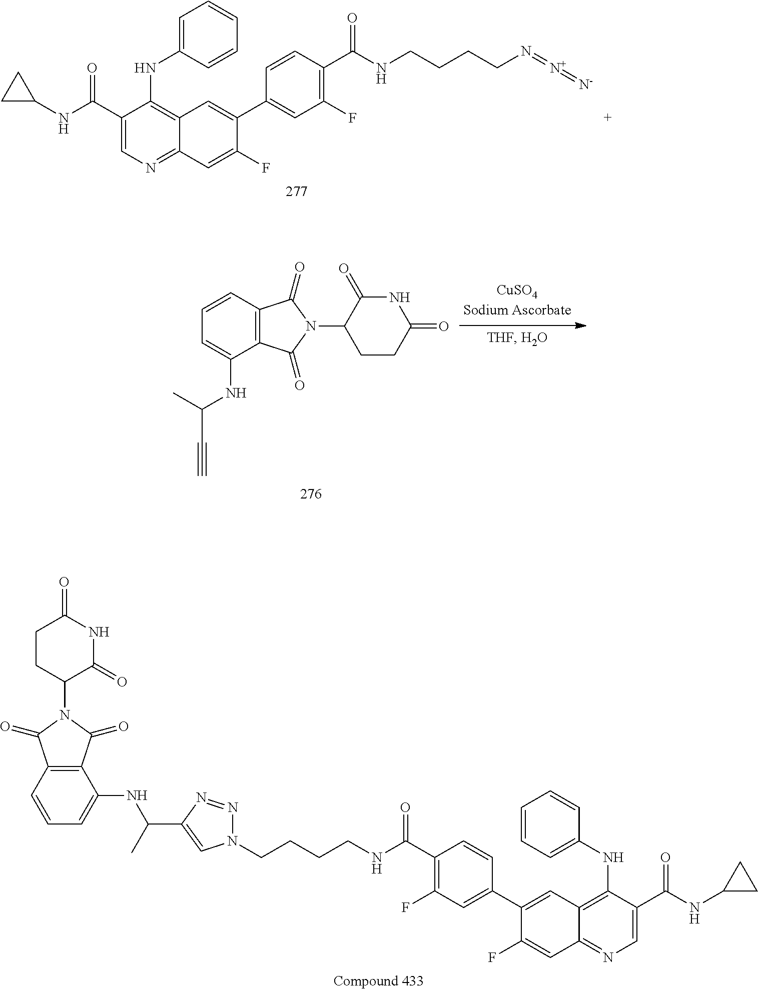 [SMILES: [N-]=[N+]=NCCCCNC(=O)c1ccc(-c2cc3c(Nc4ccccc4)c(C(=O)NC4CC4)cnc3cc2F)cc1F ; C#CC(C)Nc1cccc2c1C(=O)N(C1CCC(=O)NC1=O)C2=O ; CC(Nc1cccc2c1C(=O)N(C1CCC(=O)NC1=O)C2=O)c1cn(CCCCNC(=O)c2ccc(-c3cc4c(Nc5ccccc5)c(C(=O)NC5CC5)cnc4cc3F)cc2F)nn1]

To a stirred solution of 6-(4-((4-azidobutyl)carbamoyl)-3-fluorophenyl)-N-cyclopropyl-7-fluoro-4-(phenylamino)quinoline-3-carboxamide (277, 30 mg, 54.00 μmol) in Water (1 mL) and THF (3 mL) was added 2-(2,6-dioxo-3-piperidyl)-4-(1-methylprop-2-ynylamino)isoindoline-1,3-dione (276, 21.08 mg, 64.80 mol), copper sulfate (17.24 mg, 108.00 mol, 4.79 uL) and (+)-Sodium L-ascorbate (21.40 mg, 108.00 mol) at room temperature. The reaction mixture was stirred for 16 h at room temperature. Water was added to the reaction mixture and then extracted with ethyl acetate. Then combined organics were dried over anhydrous sodium sulfate and then concentrated to yield the crude, which was purified by Prep-HPLC to yield 4-anilino-N-cyclopropyl-6-[4-[4-[4-[1-[[2-(2,6-dioxo-3-piperidyl)-1,3-dioxo-isoindolin-4-yl]amino]ethyl]triazol-1-yl]butylcarbamoyl]-3-fluoro-phenyl]-7-fluoro-quinoline-3-carboxamide (Compound 433, 2.5 mg, 2.45 mol, 4.53% yield) as light brown colored solid. ¹H NMR (400 MHz, DMSO-d₆) δ 11.10 (s, 1H), 8.82 (s, 1H), 8.64 (d, J=7.6 Hz, 1H), 8.60 (d, J=3.4 Hz, 1H), 8.44 (t, J=6.1 Hz, 1H), 8.09 (d, J=1.7 Hz, 1H), 7.88 (d, J=10.8 Hz, 1H), 7.72 (t, J=7.7 Hz, 1H), 7.57 (t, J=7.9 Hz, 1H), 7.53-7.40 (m, 4H), 7.30 (t, J=7.4 Hz, 1H), 7.24 (d, J=7.8 Hz, 2H), 7.17 (dd, J=8.8, 4.6 Hz, 1H), 7.06 (dd, J=7.2, 1.7 Hz, 1H), 6.68 (d, J=7.7 Hz, 1H), 5.13-5.01 (m, 2H), 4.41-4.33 (m, 2H), 3.28 (q, J=6.7 Hz, 2H), 2.95-2.80 (m, 1H), 2.63-2.55 (m, 1H), 2.31 (d, J=11.9 Hz, 2H), 2.07-1.98 (m, 1H), 1.91-1.82 (m, 2H), 1.58 (d, J=6.6 Hz, 3H), 1.52-1.43 (m, 2H), 0.52 (q, J=6.3, 5.8 Hz, 2H), 0.28-0.22 (m, 2H). LCMS (ES+): m/z 881 [M+H]⁺

### Synthesis of Compound 434

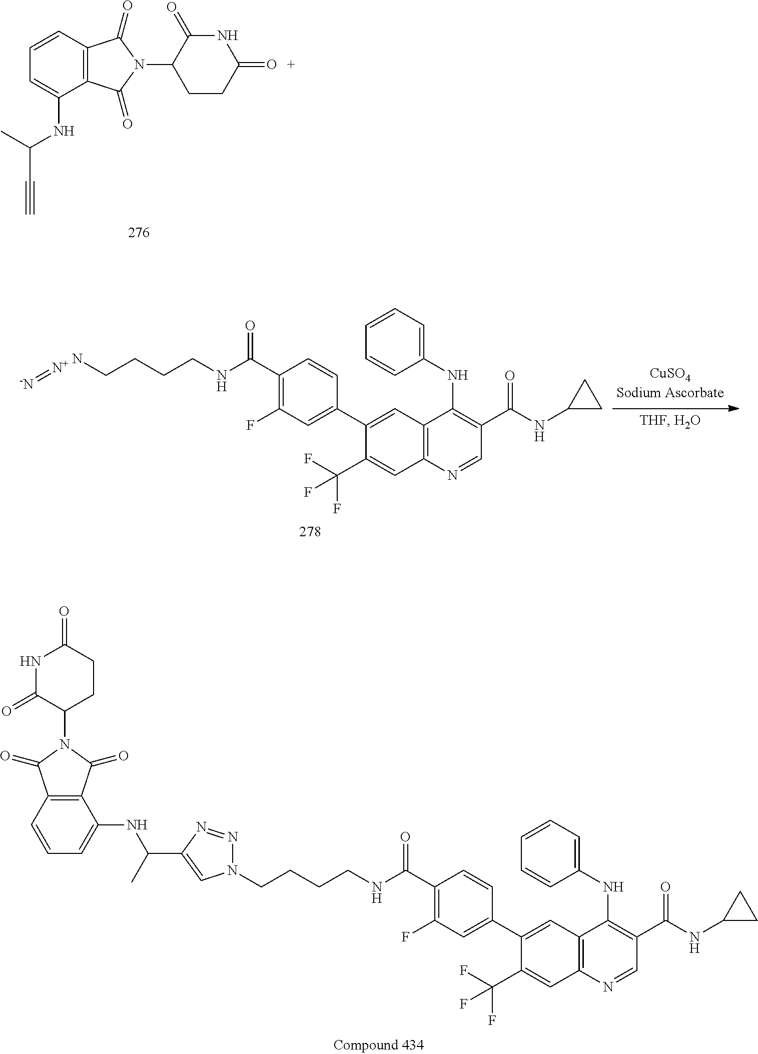

A round bottom flask was charged with a solution of 2-(2,6-dioxo-3-piperidyl)-4-(1-methylprop-2-ynylamino)isoindoline-1,3-dione (276, 51.57 mg, 158.52 μmol) in Water (1 mL) and THF (4 mL), 6-(4-((4-azidobutyl)carbamoyl)-3-fluorophenyl)-N-cyclopropyl-4-(phenylamino)-7-(trifluoromethyl)quinoline-3-carboxamide (278, 80 mg, 132.10 μmol), Copper(II) sulfate (42.17 mg, 264.21 μmol, 11.71 uL) and Sodium ascorbate (52.34 mg, 264.21 μmol) were added at room temperature. The reaction mixture was stirred for 16 h at room temperature and the reaction mixture was filtered through Celite® and the filtrate was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-N-cyclopropyl-6-[4-[4-[4-[1-[[2-(2,6-dioxo-3-piperidyl)-1,3-dioxo-isoindolin-4-yl]amino]ethyl]triazol-1-yl]butylcarbamoyl]-3-fluoro-phenyl]-7-(trifluoromethyl)quinoline-3-carboxamide (Compound 434, 5.69 mg, 5.64 μmol, 4.27% yield) as yellow solid. LCMS (ES+): m/z 931 [M+H]⁺

### Synthesis of Compound 435

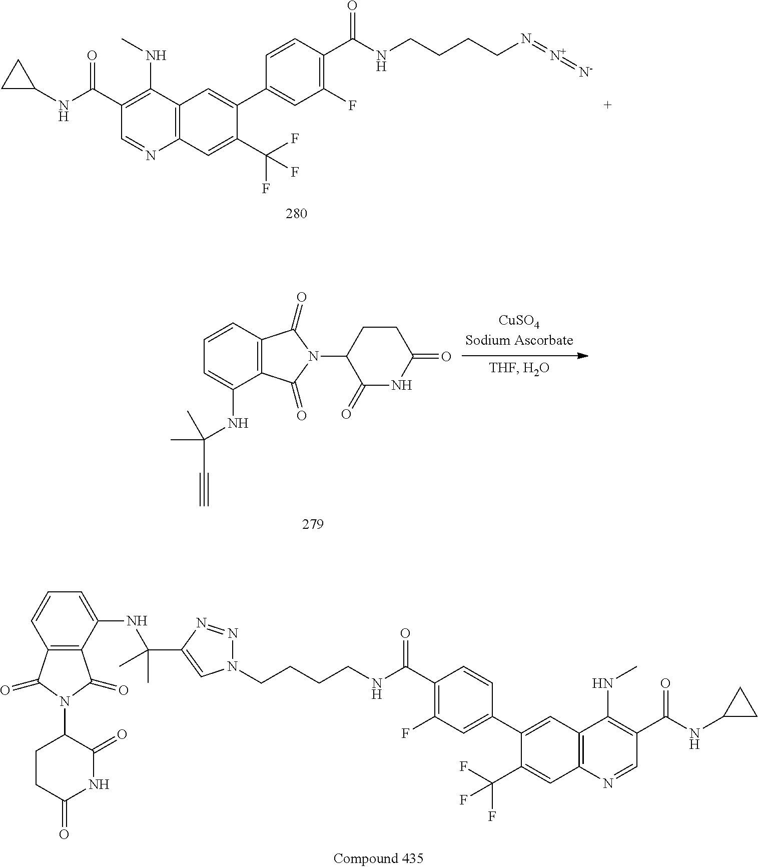 [SMILES: CNc1c(C(=O)NC2CC2)cnc2cc(C(F)(F)F)c(-c3ccc(C(=O)NCCCCN=[N+]=[N-])c(F)c3)cc12 ; C#CC(C)(C)Nc1cccc2c1C(=O)N(C1CCC(=O)NC1=O)C2=O ; CNc1c(C(=O)NC2CC2)cnc2cc(C(F)(F)F)c(-c3ccc(C(=O)NCCCCn4cc(C(C)(C)Nc5cccc6c5C(=O)N(C5CCC(=O)NC5=O)C6=O)nn4)c(F)c3)cc12]

To a stirred solution of 6-[4-(4-azidobutylcarbamoyl)-3-fluoro-phenyl]-N-cyclopropyl-4-(methylamino)-7-(trifluoromethyl)quinoline-3-carboxamide (280, 40 mg, 73.60 μmol) and 4-(1,1-dimethylprop-2-ynylamino)-2-(2,6-dioxo-3-piperidyl)isoindoline-1,3-dione (279, 27.47 mg, 80.95 μmol) in THF (5 mL) and Water (1 mL) was added Sodium ascorbate (29.16 mg, 147.19 μmol) and Copper(II) sulfate (23.49 mg, 147.19 μmol, 6.53 uL). The resulting mixture was stirred for 16 hr at 25° C. The resulting mixture was diluted with water (10 mL) and extracted with ethyl acetate (3×20 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[4-[4-[4-[1-[[2-(2,6-dioxo-3-piperidyl)-1,3-dioxo-isoindolin-4-yl]amino]-1-methyl-ethyl]triazol-1-yl]butylcarbamoyl]-3-fluoro-phenyl]-4-(methylamino)-7-(trifluoromethyl)quinoline-3-carboxamide (Compound 435, 30 mg, 29.56 μmol, 40.17% yield) as an yellow solid. ¹H NMR (400 MHz, Methanol-d₄) δ 8.68 (s, 1H), 8.49 (s, 1H), 8.27 (s, 2H), 7.96 (d, J=4.1 Hz, 2H), 7.78 (t, J=7.4 Hz, 1H), 7.38-7.27 (m, 3H), 6.92 (d, J=7.1 Hz, 1H), 6.53 (d, J=8.6 Hz, 1H), 5.04 (dd, J=12.4, 5.5 Hz, 1H), 4.47 (t, J=6.6 Hz, 2H), 3.45-3.39 (m, 2H), 2.97-2.89 (m, 1H), 2.87-2.78 (m, 1H), 2.76-2.63 (m, 2H), 2.15-2.04 (m, 1H), 2.03-1.93 (m, 2H), 1.79 (d, J=4.6 Hz, 5H), 1.61-1.51 (m, 2H), 0.91-0.81 (m, 2H), 0.72-0.65 (m, 2H). LCMS (ES+): m/z 883 [M+H]⁺

### Synthesis of Compound 436

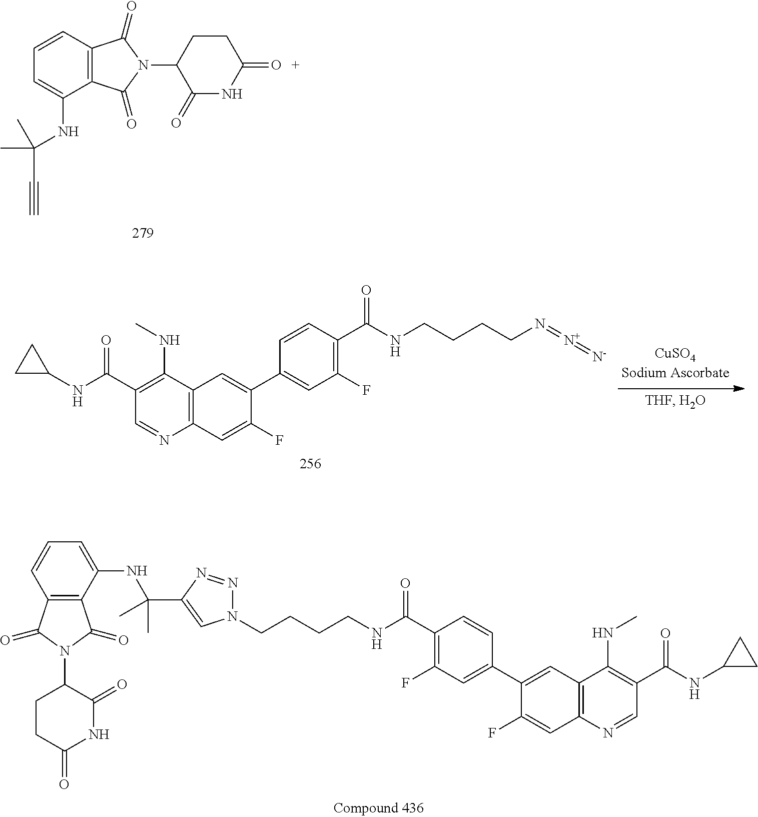 [SMILES: C#CC(C)(C)Nc1cccc2c1C(=O)N(C1CCC(=O)NC1=O)C2=O ; CNc1c(C(=O)NC2CC2)cnc2cc(F)c(-c3ccc(C(=O)NCCCCN=[N+]=[N-])c(F)c3)cc12 ; CNc1c(C(=O)NC2CC2)cnc2cc(F)c(-c3ccc(C(=O)NCCCCn4cc(C(C)(C)Nc5cccc6c5C(=O)N(C5CCC(=O)NC5=O)C6=O)nn4)c(F)c3)cc12]

To a solution of 4-(1,1-dimethylprop-2-ynylamino)-2-(2,6-dioxo-3-piperidyl)isoindoline-1,3-dione (279, 30 mg, 88.41 μmol) and 6-[4-(4-azidobutylcarbamoyl)-3-fluoro-phenyl]-N-cyclopropyl-7-fluoro-4-(methylamino)quinoline-3-carboxamide (256, 47.99 mg, 97.25 μmol) in THF (5 mL) and Water (1 mL). Added Copper (II) sulfate (28.22 mg, 176.81 μmol, 7.84 uL) and (+)-Sodium L-ascorbate (35.03 mg, 176.81 μmol). The resulting mixture was stirred for 16 hr at RT. The mixture was diluted with water and extracted with ethyl acetate (3×15 mL). The combined organic extracts were washed with water and brine solution, dried over anhydrous sodium sulfate, filtered and evaporated the solvent completely under reduced pressure. The resulting crude was purified by reverse phase prep HPLC to yield N-cyclopropyl-6-[4-[4-[4-[1-[[2-(2,6-dioxo-3-piperidyl)-1,3-dioxo-isoindolin-4-yl]amino]-1-methyl-ethyl]triazol-1-yl]butylcarbamoyl]-3-fluoro-phenyl]-7-fluoro-4-(methylamino)quinoline-3-carboxamide (Compound 436, 35 mg, 41.36 μmol, 46.79% yield) as an yellow solid. ¹H NMR (400 MHz, DMSO-d₆) δ 11.95 (s, 1H), 11.11 (s, 1H), 8.56 (d, J=4.2 Hz, 1H), 8.51 (d, J=8.3 Hz, 1H), 8.46-8.39 (m, 1H), 8.34 (s, 1H), 8.11 (s, 1H), 7.94 (s, 1H), 7.72 (t, J=7.8 Hz, 1H), 7.66-7.56 (m, 3H), 7.38 (dd, J=8.6, 7.1 Hz, 1H), 6.98 (d, J=7.1 Hz, 1H), 6.90 (s, 1H), 6.60 (d, J=8.6 Hz, 1H), 5.07 (dd, J=12.9, 5.3 Hz, 1H), 4.37 (t, J=6.9 Hz, 2H), 3.27 (q, J=6.6 Hz, 2H), 2.99 (d, J=4.9 Hz, 3H), 2.94-2.80 (m, 3H), 2.63-2.56 (m, 1H), 2.08-1.98 (m, 1H), 1.89-1.80 (m, 2H), 1.73 (s, 5H), 1.43 (dt, J=13.7, 6.4 Hz, 2H), 0.74-0.67 (m, 2H), 0.58-0.53 (m, 2H). LCMS (ES+): m/z 833 [M+H]⁺

### Synthesis of Compound 437

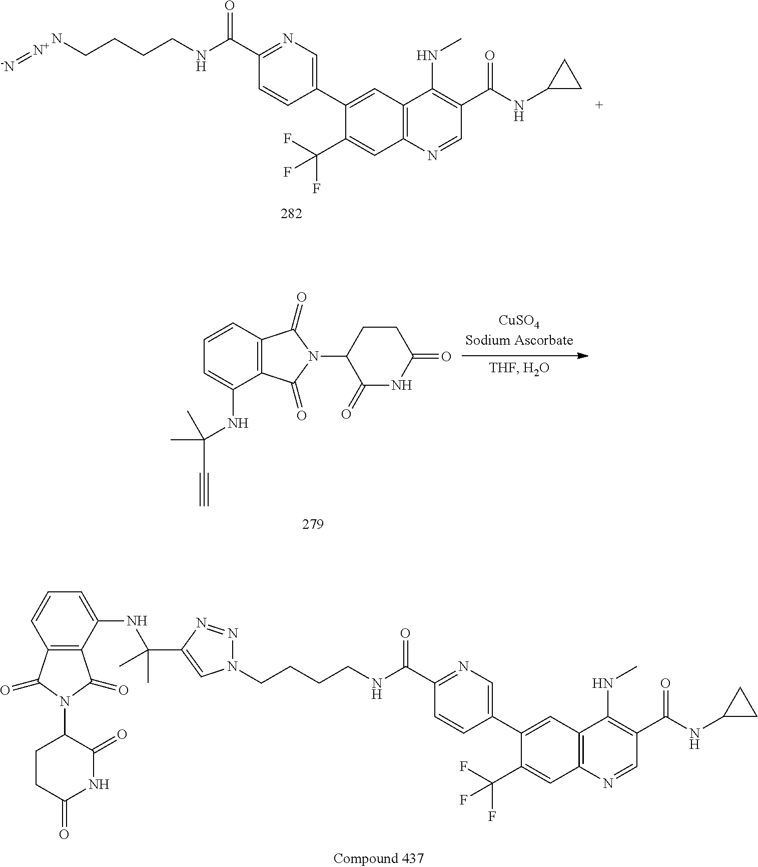

To a stirred solution of 6-[6-(4-azidobutylcarbamoyl)-3-pyridyl]-N-cyclopropyl-4-(methylamino)-7-(trifluoromethyl)quinoline-3-carboxamide (282, 30 mg, 56.98 μmol) and 4-(1,1-dimethylprop-2-ynylamino)-2-(2,6-dioxo-3-piperidyl)isoindoline-1,3-dione (279, 21.27 mg, 62.68 μmol) in THF (5 mL) and Water (1 mL) was added Sodium ascorbate (22.58 mg, 113.96 μmol) and Copper(II) sulfate (18.19 mg, 113.96 μmol, 5.05 uL). The resulting mixture was stirred for 16 hr at 25° C. The resulting mixture was diluted with water (10 mL) and extracted with ethyl acetate (3×10 mL). The combined organic extracts were washed with water and brine solution, dried over anhydrous sodium sulfate, filtered and evaporated the solvent completely under reduced pressure. The resulting crude was purified by reverse phase preparative HPLC to yield N-cyclopropyl-6-[6-[4-[4-[1-[[2-(2,6-dioxo-3-piperidyl)-1,3-dioxo-isoindolin-4-yl]amino]-1-methyl-ethyl]triazol-1-yl]butylcarbamoyl]-3-pyridyl]-4-(methylamino)-7-(trifluoromethyl)quinoline-3-carboxamide (Compound 437, 10 mg, 11.53 μmol, 20.24% yield) as an yellow solid. ¹H NMR (400 MHz, Methanol-d₄) δ 8.68 (s, 1H), 8.49 (s, 1H), 8.31 (s, 1H), 8.26 (s, 1H), 8.17 (d, J=8.0 Hz, 1H), 8.04 (dd, J=8.0, 2.2 Hz, 1H), 7.96 (d, J=1.5 Hz, 1H), 7.29 (dd, J=8.6, 7.1 Hz, 1H), 6.89 (d, J=7.1 Hz, 1H), 6.52 (d, J=8.5 Hz, 1H), 5.05 (dd, J=12.6, 5.4 Hz, 1H), 4.47 (t, J=6.7 Hz, 2H), 3.45 (t, J=7.0 Hz, 2H), 3.10 (d, J=1.6 Hz, 3H), 2.92 (tt, J=7.2, 3.8 Hz, 1H), 2.87-2.79 (m, 1H), 2.77-2.65 (m, 2H), 2.14-2.05 (m, 1H), 2.02-1.92 (m, 3H), 1.79 (s, 5H), 1.56 (p, J=7.2 Hz, 2H), 0.85 (td, J=7.2, 5.1 Hz, 2H), 0.70-0.63 (m, 2H). LCMS (ES+): m/z 866 [M+H]⁺

### Synthesis of Compound 438

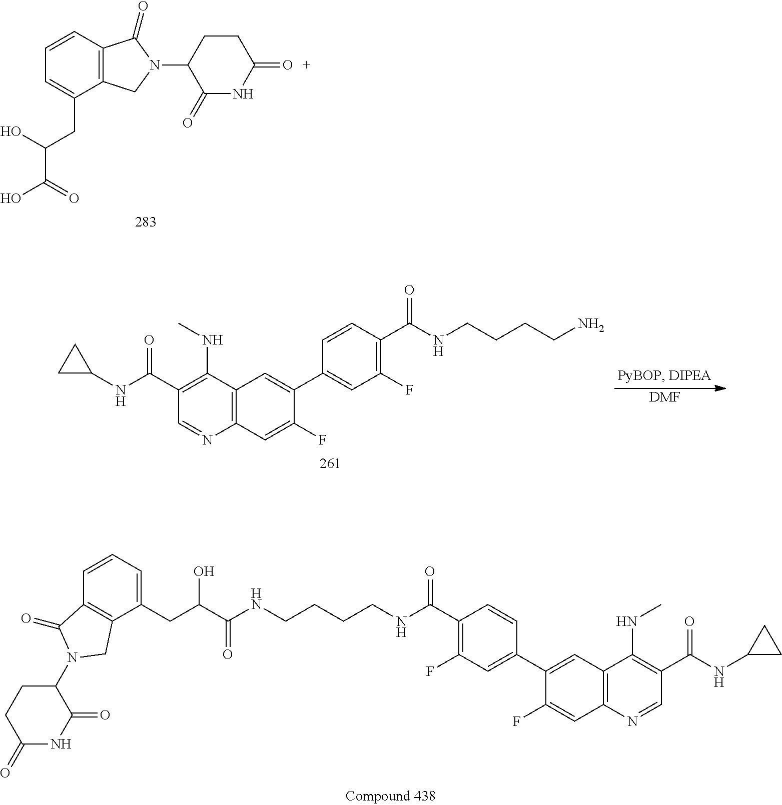

To a stirred solution of 3-[2-(2,6-dioxo-3-piperidyl)-1-oxo-isoindolin-4-yl]-2-hydroxy-propanoic acid (283, 47.06 mg, 141.62 μmol) and 6-[4-(4-aminobutylcarbamoyl)-3-fluoro-phenyl]-N-cyclopropyl-7-fluoro-4-(methylamino)quinoline-3-carboxamide (261, 44.14 mg, 94.41 μmol) in DMF (5 mL) was added PyBOP (73.70 mg, 141.62 μmol) and DIPEA (122.02 mg, 944.15 μmol, 164.45 uL). The reaction mixture was stirred for 16 hr at 25° C. The volatiles were evaporated completely under reduced pressure, the resulting crude was purified by reverse phase preparative HPLC to yield pure product N-cyclopropyl-6-[4-[4-[[3-[2-(2,6-dioxo-3-piperidyl)-1-oxo-isoindolin-4-yl]-2-hydroxy-propanoyl]amino]butylcarbamoyl]-3-fluoro-phenyl]-7-fluoro-4-(methylamino)quinoline-3-carboxamide (Compound 438, 5 mg, 6.20 μmol, 6.57% yield) as a yellow colored solid. ¹H NMR (400 MHz, DMSO-d₆) δ 10.99 (s, 1H), 8.79 (d, J=4.1 Hz, 1H), 8.70 (d, J=7.7 Hz, 1H), 8.57 (s, 1H), 8.40 (t, J=5.6 Hz, 2H), 7.84-7.72 (m, 3H), 7.69-7.56 (m, 3H), 7.50-7.41 (m, 2H), 5.75-5.70 (m, 1H), 5.13 (dd, J=13.1, 5.0 Hz, 1H), 4.51 (dd, J=17.1, 6.3 Hz, 1H), 4.43-4.32 (m, 1H), 4.19-4.10 (m, 1H), 3.25 (s, 2H), 3.10 (s, 4H), 3.04 (dd, J=14.0, 4.1 Hz, 2H), 2.98-2.90 (m, 1H), 2.89-2.78 (m, 3H), 2.63-2.55 (m, 2H), 2.43-2.36 (m, 1H), 2.00 (s, 1H), 1.48-1.39 (m, 4H), 0.74 (td, J=7.1, 4.8 Hz, 2H), 0.59 (p, J=3.7, 3.1 Hz, 2H). LCMS (ES+): m/z 782 [M+H]⁺

### Synthesis of Compound 439

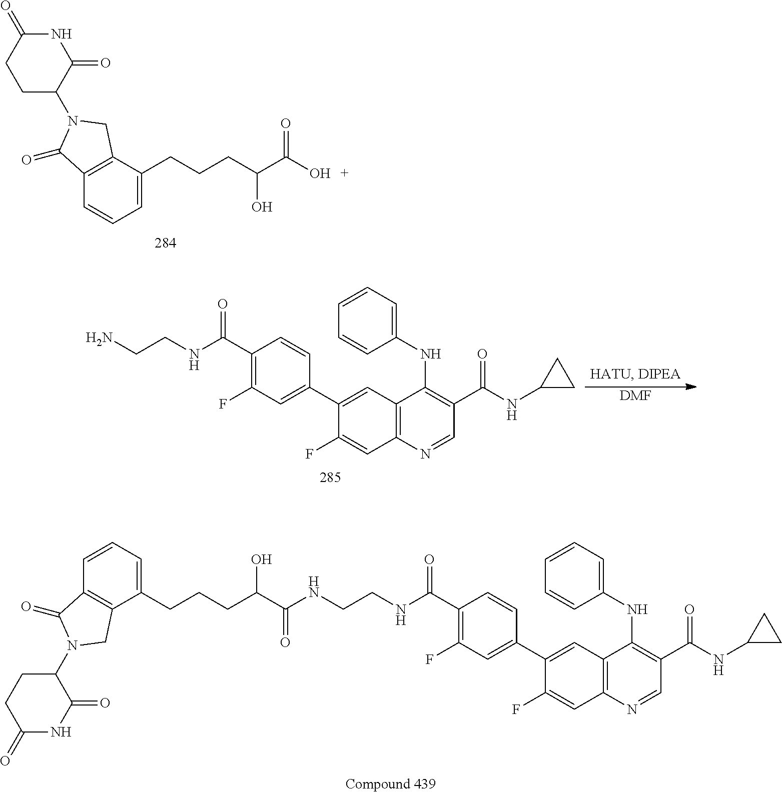

An oven dried round bottom flask was charged with a solution of 5-[2-(2,6-dioxo-3-piperidyl)-1-oxo-isoindolin-4-yl]-2-hydroxy-pentanoic acid (284, 50 mg, 138.75 μmol) in DMF (3 mL), 6-[4-(2-aminoethylcarbamoyl)-3-fluoro-phenyl]-4-anilino-N-cyclopropyl-7-fluoro-quinoline-3-carboxamide (285, 83.50 mg, 166.50 μmol), DIPEA (89.66 mg, 693.75 μmol, 120.84 uL) and HATU (63.31 mg, 166.50 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-N-cyclopropyl-6-[4-[2-[[5-[2-(2,6-dioxo-3-piperidyl)-1-oxo-isoindolin-4-yl]-2-hydroxy-pentanoyl]amino]ethylcarbamoyl]-3-fluoro-phenyl]-7-fluoro-quinoline-3-carboxamide as yellow solid. 4-anilino-N-cyclopropyl-6-[4-[2-[[5-[2-(2,6-dioxo-3-piperidyl)-1-oxo-isoindolin-4-yl]-2-hydroxy-pentanoyl]amino]ethylcarbamoyl]-3-fluoro-phenyl]-7-fluoro-quinoline-3-carboxamide (Compound 439, 24.37 mg, 27.37 μmol, 19.72% yield). ¹H NMR (400 MHz, DMSO-d₆) δ 11.06 (s, 1H), 10.99 (d, J=6.9 Hz, 1H), 8.82 (s, 1H), 8.64 (d, J=7.5 Hz, 1H), 8.58 (d, J=3.6 Hz, 1H), 8.44-8.36 (m, 1H), 7.93 (t, J=5.6 Hz, 1H), 7.87 (d, J=10.9 Hz, 1H), 7.75 (td, J=7.8, 3.6 Hz, 1H), 7.54 (ddd, J=8.5, 6.3, 5.0 Hz, 2H), 7.50-7.44 (m, 2H), 7.44-7.40 (m, 3H), 7.30 (t, J=7.4 Hz, 1H), 7.23 (d, J=7.8 Hz, 2H), 5.12 (dd, J=13.2, 5.3 Hz, 1H), 4.44 (dd, J=17.2, 3.3 Hz, 1H), 4.29 (d, J=17.2 Hz, 1H), 3.92-3.87 (m, 2H), 3.39-3.25 (m, 5H), 2.91 (ddd, J=18.0, 13.4, 5.5 Hz, 1H), 2.65-2.56 (m, 2H), 2.44-2.38 (m, 2H), 2.31-2.25 (m, 1H), 2.04-1.95 (m, 1H), 1.74-1.60 (m, 3H), 1.59-1.48 (m, 1H), 0.55-0.48 (m, 2H), 0.28-0.21 (m, 2H). LCMS (ES+): m/z 844 [M+H]⁺

### Synthesis of Compound 440

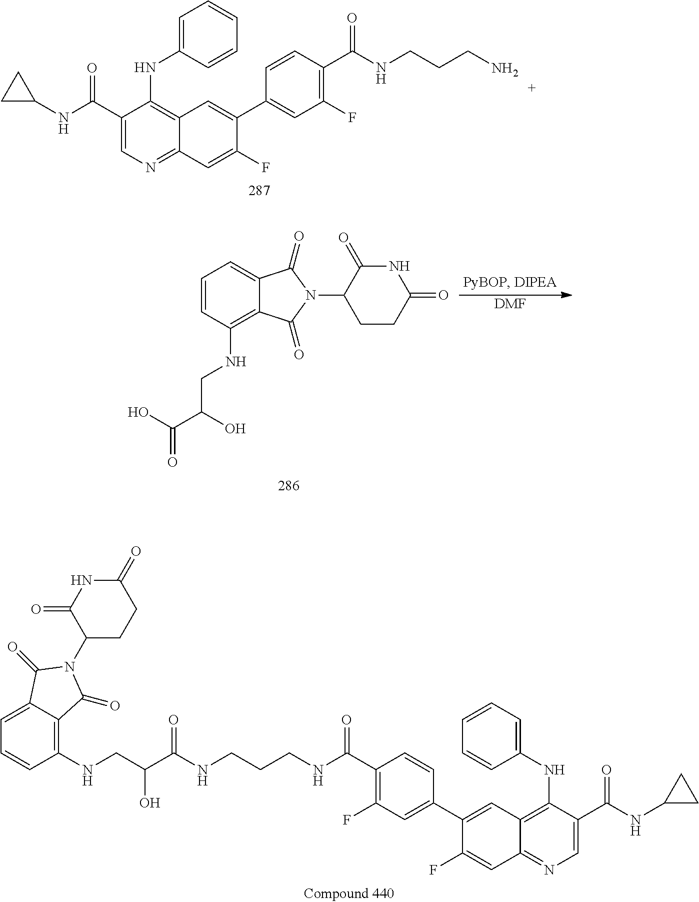

To a stirred solution of 6-[4-(3-aminopropylcarbamoyl)-3-fluoro-phenyl]-4-anilino-N-cyclopropyl-7-fluoro-quinoline-3-carboxamide (287, 100 mg, 193.97 μmol) and 3-[[2-(2,6-dioxo-3-piperidyl)-1,3-dioxo-isoindolin-4-yl]amino]-2-hydroxy-propanoic acid (286, 70.08 mg, 193.97 μmol) in N,N-Dimethylformamide (3 mL) was added N,N-Diisopropylethylamine (75.20 mg, 581.90 μmol, 101.35 uL) followed by PyBOP (151.41 mg, 290.95 μmol). The reaction mixture was stirred at room temperature for 16 hours. Water was added to the reaction mixture and then extracted with ethyl acetate. The combined organics were dried over anhydrous sodium sulfate and then concentrated to yield the crude, which was purified by prep HPLC to yield 4-anilino-N-cyclopropyl-6-[4-[3-[[3-[[2-(2,6-dioxo-3-piperidyl)-1,3-dioxo-isoindolin-4-yl]amino]-2-hydroxy-propanoyl]amino]propylcarbamoyl]-3-fluoro-phenyl]-7-fluoro-quinoline-3-carboxamide (Compound 440, 40 mg, 46.29 μmol, 23.87% yield) as light yellow colored solid. ¹H NMR (400 MHz, DMSO-d₆) δ 11.17-11.06 (m, 2H), 8.82 (s, 1H), 8.68 (d, J=7.5 Hz, 1H), 8.58 (d, J=3.5 Hz, 1H), 8.44-8.36 (m, 1H), 8.08 (t, J=6.1 Hz, 1H), 7.87 (d, J=10.8 Hz, 1H), 7.77 (t, J=7.8 Hz, 1H), 7.58 (t, J=8.6, 7.1 Hz, 1H), 7.51 (t, J=11.2 Hz, 2H), 7.44 (t, J=7.7 Hz, 2H), 7.31 (t, J=7.4 Hz, 1H), 7.24 (d, J=7.8 Hz, 2H), 7.15 (d, J=8.6 Hz, 1H), 7.04 (d, J=7.0 Hz, 1H), 6.68 (t, J=6.1 Hz, 1H), 5.05 (dd, J=12.9, 5.4 Hz, 1H), 4.13 (t, J=5.3 Hz, 1H), 3.63-3.44 (m, 3H), 3.29-3.10 (m, 4H), 2.87 (ddd, J=18.0, 13.8, 5.4 Hz, 1H), 2.60-2.55 (m, 1H), 2.31-2.23 (m, 1H), 2.06-1.97 (m, 1H), 1.69-1.57 (m, 2H), 0.56-0.46 (m, 2H), 0.23 (p, J=4.7 Hz, 2H). LCMS (ES+): m/z 859 [M+H]⁺

### Synthesis of Compound 441

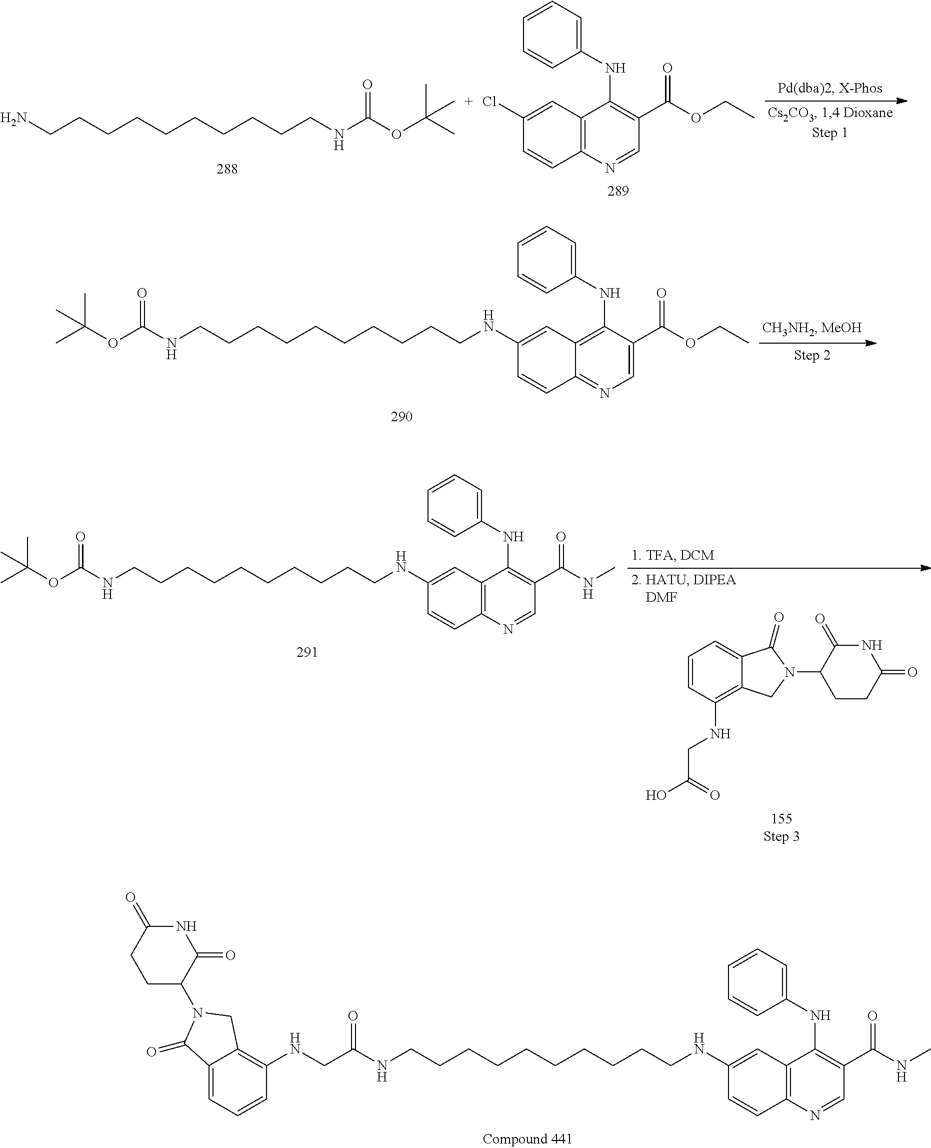

Step 1: An oven dried pressure tube was charged with a solution of ethyl 4-anilino-6-chloro-quinoline-3-carboxylate (289, 150 mg, 459.03 μmol) in 1,4-Dioxane (5 mL), tert-butyl N-(10-aminodecyl)carbamate (288, 137.56 mg, 504.93 μmol) and cesium carbonate (224.34 mg, 688.54 μmol) were added. The reaction mixture was purged with nitrogen for 5 minutes, then 2-Dicyclohexylphosphino-2′,4′,6′-triisopropylbiphenyl (43.77 mg, 91.81 μmol) and Pd₂(dba)₃ (42.03 mg, 45.90 μmol) were added. The reaction mixture was heated to 100° C. for 16 h and cooled to room temperature. The reaction mixture was diluted with water (5 mL) and the product was extracted with ethyl acetate (2×20 mL). The combined organic layer was dried over anhydrous sodium sulfate, filtered and concentrated under reduced pressure. The crude mixture was purified by column chromatography on silica (3% Methanol \Dichloromethane) to yield ethyl 4-anilino-6-[10-(tert-butoxycarbonylamino)decylamino]quinoline-3-carboxylate (290, 240 mg, 407.38 μmol, 88.75% yield) as yellow solid. LCMS (ES+): m/z 563 [M+H]⁺

Step 2: An oven dried pressure tube was charged with a solution of ethyl 4-anilino-6-[10-(tert-butoxycarbonylamino)decylamino]quinoline-3-carboxylate (290, 800 mg, 1.42 mmol) in Methyl amine in methanol (1.42 mmol, 10 mL) and the reaction mixture was heated to 80° C. for 16 h. The reaction mixture was cooled to room temperature and the reaction mixture was concentrated under reduced pressure. The crude mixture was purified by column chromatography on silica (5% Methanol \Dichloromethane) to yield tert-butyl N-[10-[[4-anilino-3-(methylcarbamoyl)-6-quinolyl]amino]decyl]carbamate (291, 500 mg, 715.23 μmol, 50.31% yield) as yellow solid. LCMS (ES+): m/z 548 [M+H]⁺

Step 3: An oven dried round bottom flask was charged with a solution of tert-butyl N-[10-[[4-anilino-3-(methylcarbamoyl)-6-quinolyl]amino]decyl]carbamate (291, 124.29 mg, 226.92 μmol) in Dichloromethane (3 mL), Trifluoroacetic acid (1.85 g, 16.26 mmol, 1.25 mL) was added at room temperature. The reaction mixture was stirred for an hour at room temperature and the reaction mixture was concentrated under reduced pressure. To a solution of crude product in DMF (3 mL), 2-[[2-(2,6-dioxo-3-piperidyl)-1-oxo-isoindolin-4-yl]amino]acetic acid (155, 60 mg, 189.10 μmol), DIPEA (122.20 mg, 945.49 μmol, 164.68 uL) and HATU (86.28 mg, 226.92 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[10-[[2-[[2-(2,6-dioxo-3-piperidyl)-1-oxo-isoindolin-4-yl]amino]acetyl]amino]decylamino]-N-methyl-quinoline-3-carboxamide (Compound 441, 3 mg, 3.98 μmol, 2.10% yield) as yellow solid. ¹H NMR (400 MHz, DMSO-d₆) δ 14.35 (s, 1H), 11.02 (s, 1H), 10.79 (s, 1H), 8.51 (s, 1H), 8.47 (q, J=4.5 Hz, 1H), 7.93 (t, J=5.8 Hz, 1H), 7.72 (d, J=9.2 Hz, 1H), 7.43-7.35 (m, 3H), 7.28-7.23 (m, 2H), 7.21 (d, J=7.8 Hz, 2H), 6.97 (d, J=7.4 Hz, 1H), 6.93 (d, J=2.3 Hz, 1H), 6.66 (s, 1H), 6.54 (d, J=8.0 Hz, 1H), 5.12 (dd, J=13.3, 5.1 Hz, 1H), 4.28 (d, J=17.1 Hz, 1H), 4.18 (d, J=17.1 Hz, 1H), 3.71 (s, 2H), 3.05 (q, J=6.7 Hz, 2H), 2.98-2.86 (m, 3H), 2.70-2.57 (m, 1H), 2.38 (d, J=4.5 Hz, 3H), 2.34-2.23 (m, 1H), 2.07-1.95 (m, 1H), 1.56-1.43 (m, 2H), 1.42-1.14 (m, 13H). LCMS (ES+): m/z 747 [M+H]⁺

### Synthesis of Compound 442

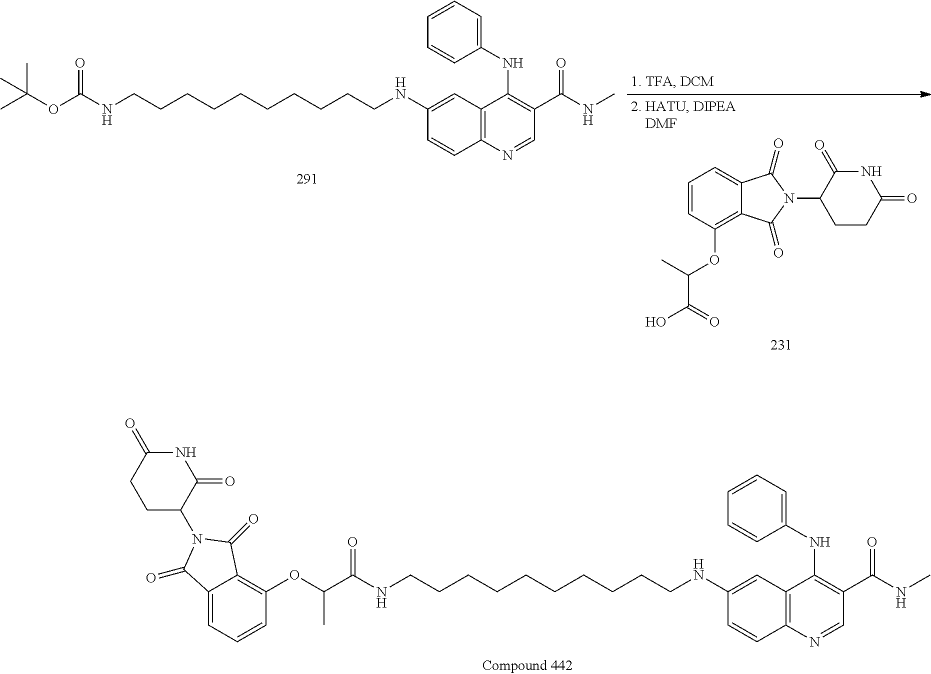 [SMILES: CNC(=O)c1cnc2ccc(NCCCCCCCCCCNC(=O)OC(C)(C)C)cc2c1Nc1ccccc1 ; CC(Oc1cccc2c1C(=O)N(C1CCC(=O)NC1=O)C2=O)C(=O)O ; CNC(=O)c1cnc2ccc(NCCCCCCCCCCNC(=O)C(C)Oc3cccc4c3C(=O)N(C3CCC(=O)NC3=O)C4=O)cc2c1Nc1ccccc1]

An oven dried round bottom flask was charged with a solution of tert-butyl N-[10-[[4-anilino-3-(methylcarbamoyl)-6-quinolyl]amino]decyl]carbamate (291, 110.72 mg, 202.14 μmol) in dichloromethane (5 mL), Trifluoroacetic acid (2.96 g, 25.96 mmol, 2 mL) was added at room temperature. The reaction mixture was stirred for an hour at room temperature and the reaction mixture was concentrated under reduced pressure. To a solution of crude product in DMF (3 mL), 2-[2-(2,6-dioxo-3-piperidyl)-1,3-dioxo-isoindolin-4-yl]oxypropanoic acid (231, 70 mg, 202.14 DIPEA (130.62 mg, 1.01 mmol, 176.04 uL) and HATU (92.23 mg, 242.57 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[10-[2-[2-(2,6-dioxo-3-piperidyl)-1,3-dioxo-isoindolin-4-yl]oxypropanoylamino]decylamino]-N-methyl-quinoline-3-carboxamide (Compound 442, 16.27 mg, 20.14 μmol, 9.96% yield) as a yellow solid. ¹H NMR (400 MHz, DMSO-d₆) δ 14.41 (s, 1H), 11.14 (s, 1H), 10.80 (s, 1H), 8.52 (s, 1H), 8.51-8.45 (m, 1H), 8.05 (t, J=5.7 Hz, 1H), 7.84-7.76 (m, 1H), 7.72 (dd, J=9.3, 1.9 Hz, 1H), 7.49 (d, J=7.3 Hz, 1H), 7.43-7.32 (m, 4H), 7.28-7.18 (m, 3H), 6.93 (s, 1H), 6.67 (s, 1H), 5.11 (dd, J=13.0, 5.3 Hz, 1H), 4.94 (q, J=6.6 Hz, 1H), 3.09 (q, J=6.7 Hz, 2H), 2.96-2.83 (m, 3H), 2.63-2.57 (m, 1H), 2.38 (d, J=4.4 Hz, 3H), 2.08-1.92 (m, 1H), 1.54-1.44 (m, 5H), 1.43-1.34 (m, 2H), 1.33-1.14 (m, 12H). LCMS (ES+): m/z 776 [M+H]⁺

### Synthesis of Compound 443

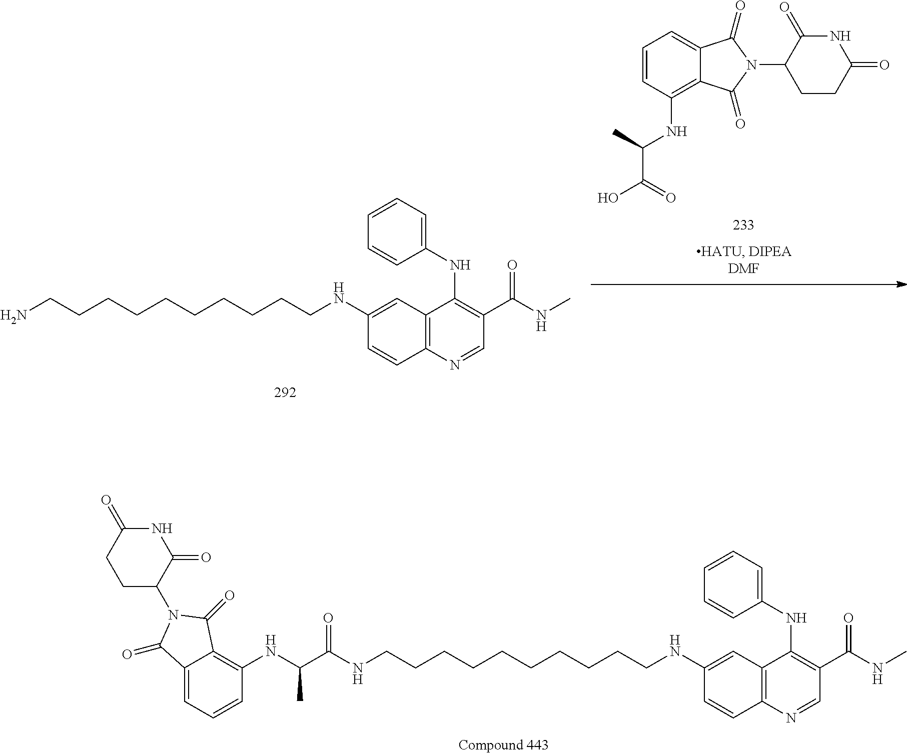 [SMILES: C[C@@H](Nc1cccc2c1C(=O)N(C1CCC(=O)NC1=O)C2=O)C(=O)O ; CNC(=O)c1cnc2ccc(NCCCCCCCCCCN)cc2c1Nc1ccccc1 ; CNC(=O)c1cnc2ccc(NCCCCCCCCCCNC(=O)[C@@H](C)Nc3cccc4c3C(=O)N(C3CCC(=O)NC3=O)C4=O)cc2c1Nc1ccccc1]

An oven dried round bottom flask was charged with a solution of (2R)-2-[[2-(2,6-dioxo-3-piperidyl)-1,3-dioxo-isoindolin-4-yl]amino]propanoic acid (233, 69.80 mg, 202.14 μmol) in DMF (3 mL), 6-(10-aminodecylamino)-4-anilino-N-methyl-quinoline-3-carboxamide (292, 108.58 mg, 242.57 μmol), DIPEA (130.62 mg, 1.01 mmol, 176.04 uL) and HATU (92.23 mg, 242.57 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[10-[[(2R)-2-[[2-(2,6-dioxo-3-piperidyl)-1,3-dioxo-isoindolin-4-yl]amino]propanoyl]amino]decylamino]-N-methyl-quinoline-3-carboxamide (Compound 443, 39.25 mg, 50.50 μmol, 24.98% yield) as yellow solid. ¹H NMR (400 MHz, DMSO-d₆) δ 14.35 (s, 1H), 11.11 (s, 1H), 10.79 (s, 1H), 8.51 (s, 1H), 8.49-8.44 (m, 1H), 8.22-8.15 (m, 1H), 7.72 (d, J=9.2 Hz, 1H), 7.60 (t, J=7.8 Hz, 1H), 7.40 (t, J=7.5 Hz, 3H), 7.25 (t, J=7.5 Hz, 1H), 7.21 (d, J=7.9 Hz, 2H), 7.08 (d, J=7.1 Hz, 1H), 6.91 (d, J=7.1 Hz, 2H), 6.73 (t, J=6.8 Hz, 1H), 6.66 (s, 1H), 5.07 (dd, J=13.0, 5.4 Hz, 1H), 4.17 (dt, J=13.6, 6.5 Hz, 1H), 3.11-3.01 (m, 2H), 2.96-2.82 (m, 3H), 2.63-2.53 (m, 2H), 2.38 (d, J=4.4 Hz, 3H), 2.06-1.96 (m, 1H), 1.54-1.43 (m, 2H), 1.42-1.33 (m, 5H), 1.23 (s, 12H). LCMS (ES+): m/z 776 [M+H]⁺

### Synthesis of Compound 444

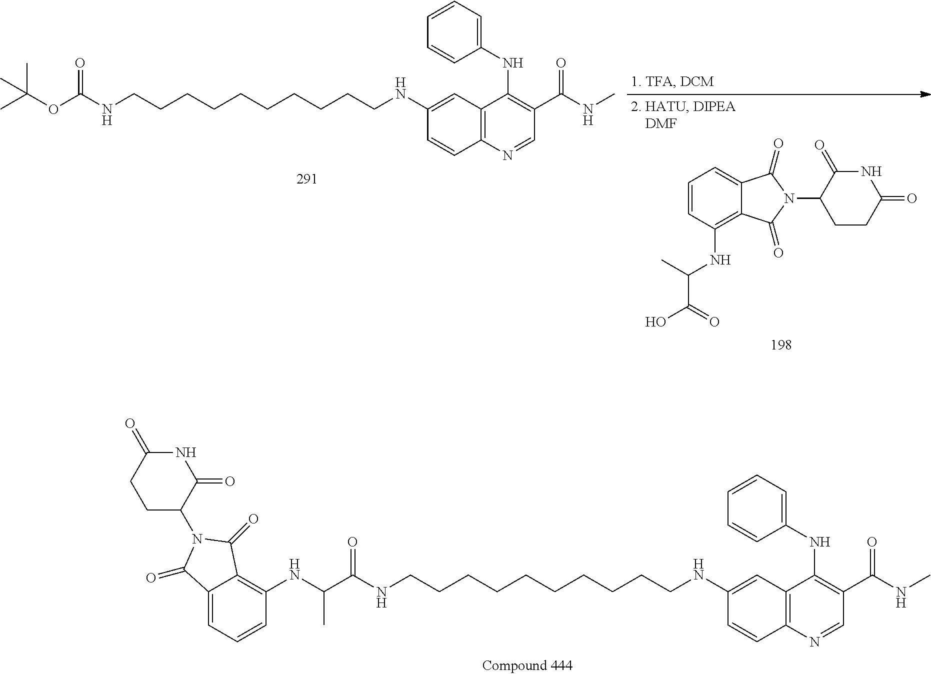 [SMILES: CNC(=O)c1cnc2ccc(NCCCCCCCCCCNC(=O)OC(C)(C)C)cc2c1Nc1ccccc1 ; CC(Nc1cccc2c1C(=O)N(C1CCC(=O)NC1=O)C2=O)C(=O)O ; CNC(=O)c1cnc2ccc(NCCCCCCCCCCNC(=O)C(C)Nc3cccc4c3C(=O)N(C3CCC(=O)NC3=O)C4=O)cc2c1Nc1ccccc1]

An oven dried round bottom flask was charged with a solution of tert-butyl N-[10-[[4-anilino-3-(methylcarbamoyl)-6-quinolyl]amino]decyl]carbamate (291, 114.21 mg, 208.51 μmol) in dichloromethane (3 mL), Trifluoroacetic acid (2.96 g, 25.96 mmol, 2 mL) was added at room temperature. The reaction mixture was stirred for an hour at room temperature and the reaction mixture was concentrated under reduced pressure. To a solution of crude product in DMF (2 mL), 2-[[2-(2,6-dioxo-3-piperidyl)-1,3-dioxo-isoindolin-4-yl]amino]propanoic acid (198, 60 mg, 173.76 μmol), DIPEA (112.28 mg, 868.79 μmol, 151.32 uL) and HATU (79.28 mg, 208.51 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[10-[2-[[2-(2,6-dioxo-3-piperidyl)-1,3-dioxo-isoindolin-4-yl]amino]propanoylamino]decylamino]-N-methyl-quinoline-3-carboxamide (Compound 444, 7.41 mg, 9.45 μmol, 5.44% yield) as yellow solid. ¹H NMR (400 MHz, DMSO-d₆) δ 11.16-11.07 (m, 1H), 9.82 (s, 1H), 8.63-8.58 (m, 1H), 8.58 (s, 1H), 8.18 (t, J=5.6 Hz, 1H), 7.67-7.55 (m, 2H), 7.24-7.17 (m, 2H), 7.12 (dd, J=9.2, 2.4 Hz, 1H), 7.08 (d, J=7.0 Hz, 1H), 6.95-6.89 (m, 2H), 6.86-6.80 (m, 2H), 6.73 (t, J=6.9 Hz, 1H), 6.38 (d, J=2.5 Hz, 1H), 6.10 (t, J=5.4 Hz, 1H), 5.07 (dd, J=12.8, 5.5 Hz, 1H), 4.24-4.11 (m, 1H), 3.07 (q, J=6.6 Hz, 2H), 2.88 (ddd, J=18.7, 13.9, 5.5 Hz, 1H), 2.73-2.69 (m, 3H), 2.68-2.59 (m, 3H), 2.08-1.97 (m, 1H), 1.44-1.34 (m, 5H), 1.32-1.27 (m, 2H), 1.21 (s, 8H), 1.15 (s, 4H). LCMS (ES+): m/z 775 [M+H]⁺

### Synthesis of Compound 445

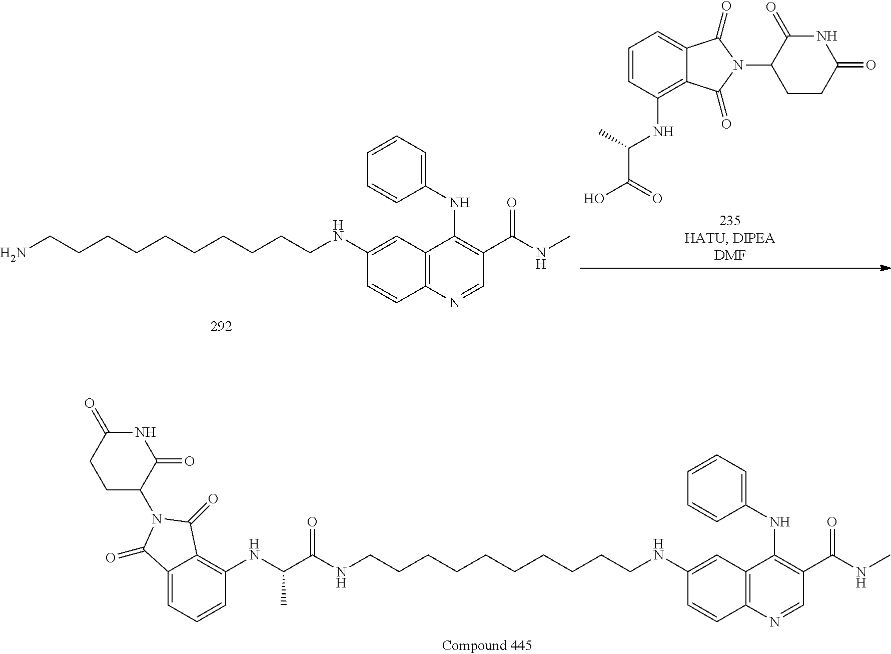 [SMILES: CNC(=O)c1cnc2ccc(NCCCCCCCCCCN)cc2c1Nc1ccccc1 ; C[C@H](Nc1cccc2c1C(=O)N(C1CCC(=O)NC1=O)C2=O)C(=O)O ; CNC(=O)c1cnc2ccc(NCCCCCCCCCCNC(=O)[C@H](C)Nc3cccc4c3C(=O)N(C3CCC(=O)NC3=O)C4=O)cc2c1Nc1ccccc1]

An oven dried round bottom flask was charged with a solution of (2S)-2-[[2-(2,6-dioxo-3-piperidyl)-1,3-dioxo-isoindolin-4-yl]amino]propanoic acid (235, 70 mg, 202.72 μmol) in DMF (3 mL), 6-(10-aminodecylamino)-4-anilino-N-methyl-quinoline-3-carboxamide (292, 108.89 mg, 243.26 μmol), DIPEA (131.00 mg, 1.01 mmol, 176.55 uL) and HATU (92.50 mg, 243.26 μmol) were added. The reaction mixture was stirred for 16 h at room temperature and the reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[10-[[(2S)-2-[[2-(2,6-dioxo-3-piperidyl)-1,3-dioxo-isoindolin-4-yl]amino]propanoyl]amino]decylamino]-N-methyl-quinoline-3-carboxamide (Compound 445, 58 mg, 71.36 μmol, 35.20% yield) as yellow solid. ¹H NMR (400 MHz, DMSO-d₆) δ 14.41 (s, 1H), 11.12 (s, 1H), 10.82 (s, 1H), 8.52 (s, 1H), 8.49 (q, J=4.7 Hz, 1H), 8.19 (t, J=5.6 Hz, 1H), 7.73 (d, J=9.2 Hz, 1H), 7.64-7.57 (m, 1H), 7.43-7.37 (m, 3H), 7.26 (t, J=7.4 Hz, 1H), 7.22 (d, J=7.8 Hz, 2H), 7.09 (d, J=7.1 Hz, 1H), 6.96-6.89 (m, 2H), 6.74 (t, J=6.8 Hz, 1H), 6.68 (d, J=8.2 Hz, 1H), 5.07 (dd, J=12.9, 5.4 Hz, 1H), 4.18 (dt, J=13.2, 7.0 Hz, 1H), 3.08 (q, J=6.8 Hz, 2H), 2.96-2.83 (m, 3H), 2.64-2.55 (m, 2H), 2.39 (d, J=4.5 Hz, 3H), 2.09-1.97 (m, 1H), 1.54-1.45 (m, 2H), 1.43-1.34 (m, 5H), 1.23 (s, 12H). LCMS (ES+): m/z 775 [M+H]⁺

### Synthesis of Compound 446

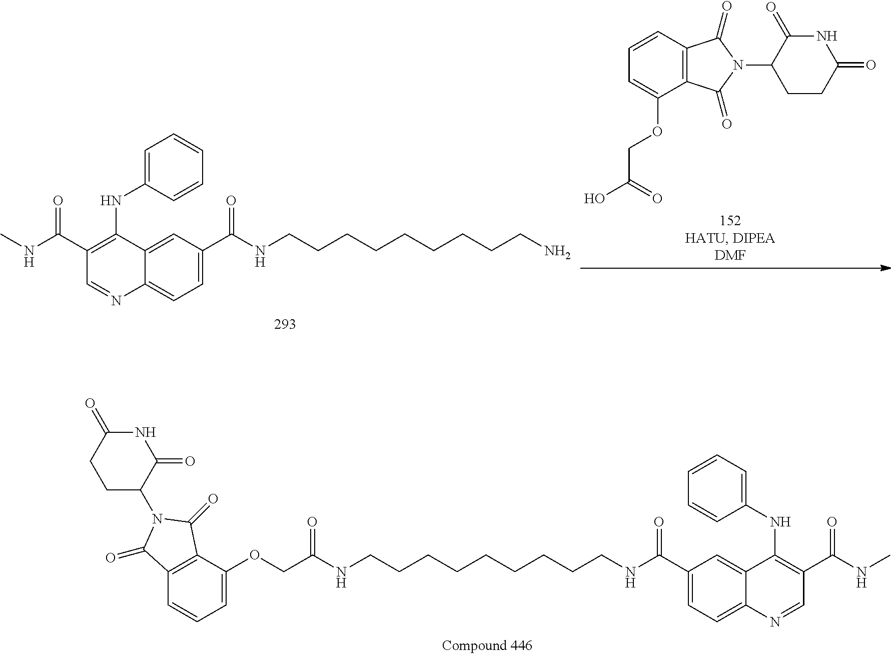 [SMILES: CNC(=O)c1cnc2ccc(C(=O)NCCCCCCCCCN)cc2c1Nc1ccccc1 ; O=C(O)COc1cccc2c1C(=O)N(C1CCC(=O)NC1=O)C2=O ; CNC(=O)c1cnc2ccc(C(=O)NCCCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of 2-[2-(2,6-dioxo-3-piperidyl)-1,3-dioxo-isoindolin-4-yl]oxyacetic acid (152, 35.99 mg, 108.32 μmol) and N6-(9-aminononyl)-4-anilino-N3-methyl-quinoline-3,6-dicarboxamide (293, 50 mg, 108.32 μmol) in anhydrous DMF (2 mL) were added DIPEA (42.00 mg, 324.96 μmol, 56.60 uL) and HATU (61.78 mg, 162.48 μmol) under nitrogen atmosphere at room temperature. The resulting mixture was stirred at room temperature for 16 h. The reaction mixture was concentrated to afford crude residue which was purified by prep-HPLC (SUNFIRE OBD C18(100×30)MM 50 Mobile phase: A: 0.1% TFA in water B: ACN to afford 4-anilino-N6-[9-[[2-[2-(2,6-dioxo-3-piperidyl)-1,3-dioxo-isoindolin-4-yl]oxyacetyl]amino]nonyl]-N3-methyl-quinoline-3,6-dicarboxamide (Compound 446, 6 mg, 7.50 μmol, 6.93% yield) as a yellow solid. ¹H NMR (400 MHz, DMSO-d₆) δ 11.12 (s, 1H), 8.95 (s, 1H), 8.81 (s, 1H), 8.66-8.59 (m, 1H), 8.50-8.41 (m, 1H), 8.29 (d, J=8.8 Hz, 1H), 8.00 (d, J=8.8 Hz, 1H), 7.92 (t, J=5.8 Hz, 1H), 7.80 (dd, J=8.5, 7.3 Hz, 1H), 7.49 (d, J=7.2 Hz, 1H), 7.37 (t, J=8.0 Hz, 3H), 7.29-7.16 (m, 3H), 5.11 (dd, J=12.9, 5.4 Hz, 1H), 4.76 (s, 2H), 3.27 (q, J=6.7 Hz, 2H), 3.13 (q, J=6.6 Hz, 2H), 2.89 (ddd, J=17.4, 14.0, 5.5 Hz, 1H), 2.63-2.55 (m, 2H), 2.04 (dd, J=9.7, 4.9 Hz, 1H), 1.58-1.48 (m, 2H), 1.46-1.37 (m, 2H), 1.33-1.21 (m, 10H). LCMS (ES+): m/z 776 [M+H]⁺

### Synthesis of Compound 447

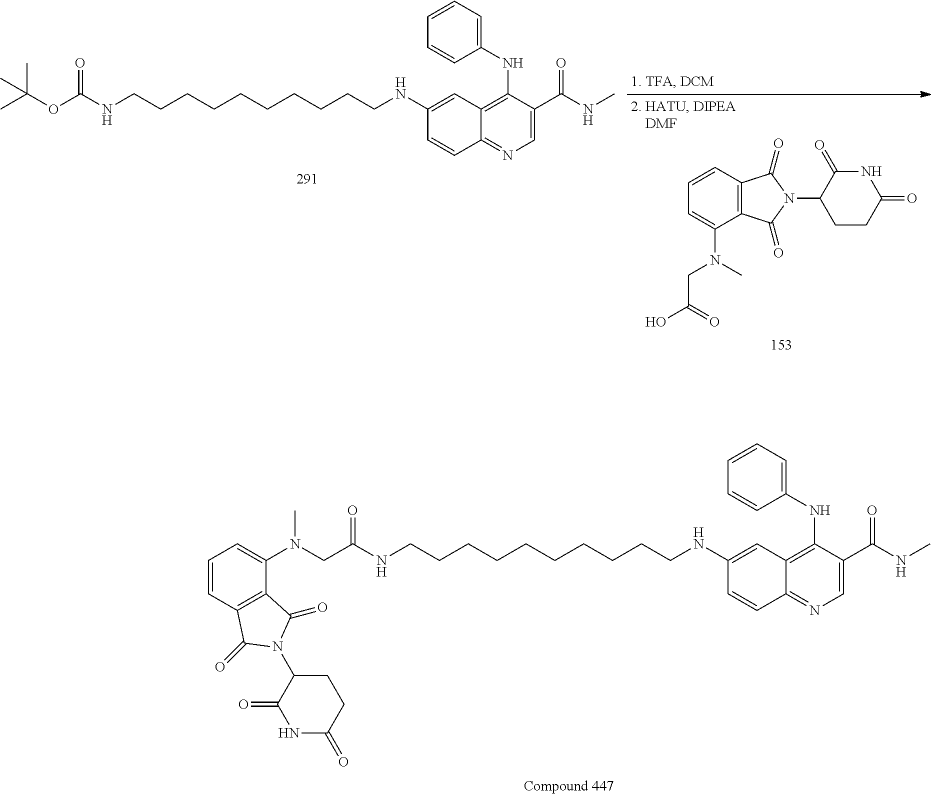 [SMILES: CNC(=O)c1cnc2ccc(NCCCCCCCCCCNC(=O)OC(C)(C)C)cc2c1Nc1ccccc1 ; CN(CC(=O)O)c1cccc2c1C(=O)N(C1CCC(=O)NC1=O)C2=O ; CNC(=O)c1cnc2ccc(NCCCCCCCCCCNC(=O)CN(C)c3cccc4c3C(=O)N(C3CCC(=O)NC3=O)C4=O)cc2c1Nc1ccccc1]

Into a 25 mL single-necked round-bottomed flask containing a well-stirred solution of tert-butyl N-[10-[[4-anilino-3-(methylcarbamoyl)-6-quinolyl]amino]decyl]carbamate (291, 75 mg, 136.93 μmol) in anhydrous DCM (5 mL) was added TFA (78.06 mg, 684.65 μmol, 52.75 uL) under nitrogen atmosphere at room temperature, the resulting mixture was stirred at room temperature for 30 minutes. The reaction mixture was concentrated under reduced pressure to afford a crude residue which was dissolved in DMF (4 mL) were added DIPEA (53.09 mg, 410.79 μmol, 71.55 uL), 2-[[2-(2,6-dioxo-3-piperidyl)-1,3-dioxo-isoindolin-4-yl]-methyl-amino]acetic acid (153, 47.28 mg, 136.93 μmol) and HATU (78.10 mg, 205.40 μmol) under nitrogen atmosphere at room temperature. The resulting mixture was stirred at room temperature for 16 h. Ice cold water (15 mL) was added and stirred for 10 minutes, the resulting solid was filtered and dried to afford a crude solid which was purified by prep-HPLC (SUNFIRE OBD C18(100×30)MM 50 Mobile phase: A: 0.1% TFA in water B: ACN to afford 4-anilino-6-[10-[[2-[[2-(2,6-dioxo-3-piperidyl)-1,3-dioxo-isoindolin-4-yl]-methyl-amino]acetyl]amino]decylamino]-N-methyl-quinoline-3-carboxamide (Compound 447, 38 mg, 46.91 μmol, 34.25% yield) as a yellow solid. ¹H NMR (400 MHz, DMSO-d₆) δ 14.37 (s, 1H), 11.08 (s, 1H), 10.80 (s, 1H), 8.51 (s, 1H), 8.50-8.44 (m, 1H), 7.90 (t, J=5.9 Hz, 2H), 7.75-7.69 (m, 1H), 7.66-7.58 (m, 1H), 7.43-7.37 (m, 3H), 7.28-7.18 (m, 5H), 6.92 (s, 1H), 6.66 (s, 1H), 5.07 (dd, J=12.9, 5.3 Hz, 1H), 4.21-4.06 (m, 2H), 3.08-3.01 (m, 2H), 3.01 (s, 3H), 2.93-2.82 (m, 3H), 2.69-2.55 (m, 2H), 2.38 (t, J=3.2 Hz, 3H), 2.33 (s, 1H), 2.05-1.96 (m, 1H), 1.53-1.44 (m, 2H), 1.41-1.33 (m, 2H), 1.24 (s, 10H). LCMS (ES+): m/z 776 [M+H]⁺

### Synthesis of Compound 448

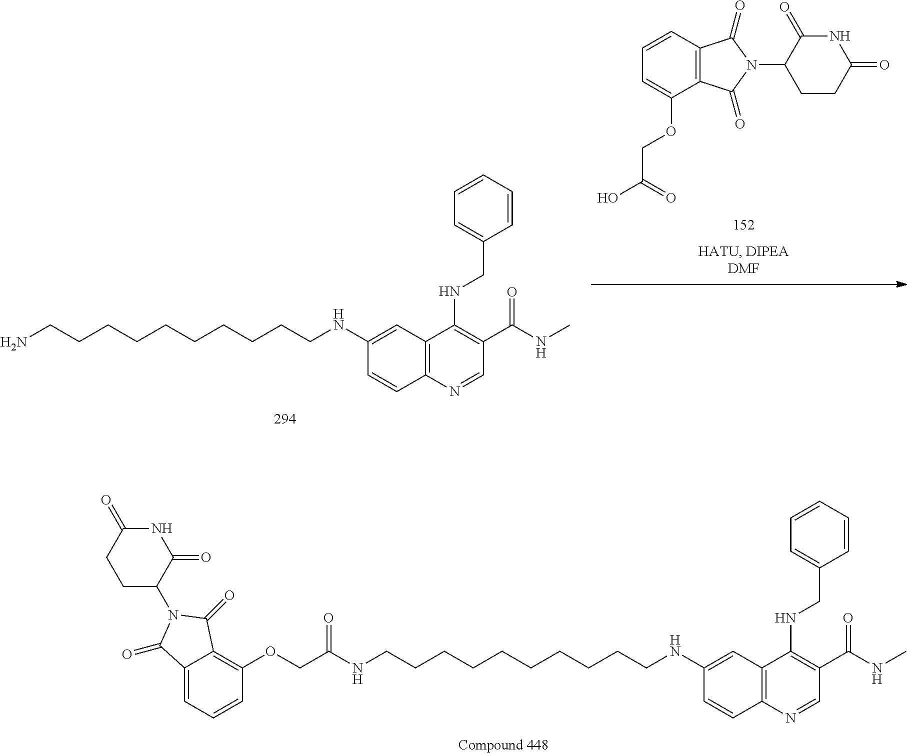 [SMILES: CNC(=O)c1cnc2ccc(NCCCCCCCCCCN)cc2c1NCc1ccccc1 ; O=C(O)COc1cccc2c1C(=O)N(C1CCC(=O)NC1=O)C2=O ; CNC(=O)c1cnc2ccc(NCCCCCCCCCCNC(=O)COc3cccc4c3C(=O)N(C3CCC(=O)NC3=O)C4=O)cc2c1NCc1ccccc1]

To a stirred solution of 2-[2-(2,6-dioxo-3-piperidyl)-1,3-dioxo-isoindolin-4-yl]oxyacetic acid (152, 100 mg, 300.96 μmol) and 6-(10-aminodecylamino)-4-(benzylamino)-N-methyl-quinoline-3-carboxamide (294, 138.94 mg, 300.96 μmol) in DMF (10.0 mL) was added DIPEA (194.49 mg, 1.50 mmol, 262.11 uL) and HATU (171.65 mg, 451.45 μmol). The resulting mixture was stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-(benzylamino)-6-[10-[[2-[2-(2,6-dioxo-3-piperidyl)-1,3-dioxo-isoindolin-4-yl]oxyacetyl]amino]decylamino]-N-methyl-quinoline-3-carboxamide (Compound 448, 70 mg, 84.47 μmol, 28.07% yield) as an yellow solid. ¹H NMR (400 MHz, DMSO-d₆) δ 13.89 (s, 1H), 11.12 (s, 1H), 8.44 (s, 1H), 8.33 (s, 1H), 7.93 (t, J=5.7 Hz, 1H), 7.80 (dd, J=8.5, 7.3 Hz, 1H), 7.64 (d, J=9.1 Hz, 1H), 7.49 (d, J=7.3 Hz, 1H), 7.41-7.32 (m, 4H), 7.31-7.26 (m, 3H), 7.18 (d, J=2.3 Hz, 1H), 6.56 (s, 1H), 5.11 (dd, J=13.0, 5.4 Hz, 1H), 4.90 (s, 2H), 4.76 (s, 2H), 3.13 (q, J=6.6 Hz, 2H), 3.04 (s, 2H), 2.89 (ddd, J=18.3, 14.1, 5.5 Hz, 1H), 2.70-2.56 (m, 5H), 2.43-2.29 (m, 1H), 2.09-1.97 (m, 1H), 1.62-1.52 (m, 2H), 1.46-1.34 (m, 4H), 1.24 (s, 8H). LCMS (ES+): m/z 776 [M+H]⁺

### Synthesis of Compound 449

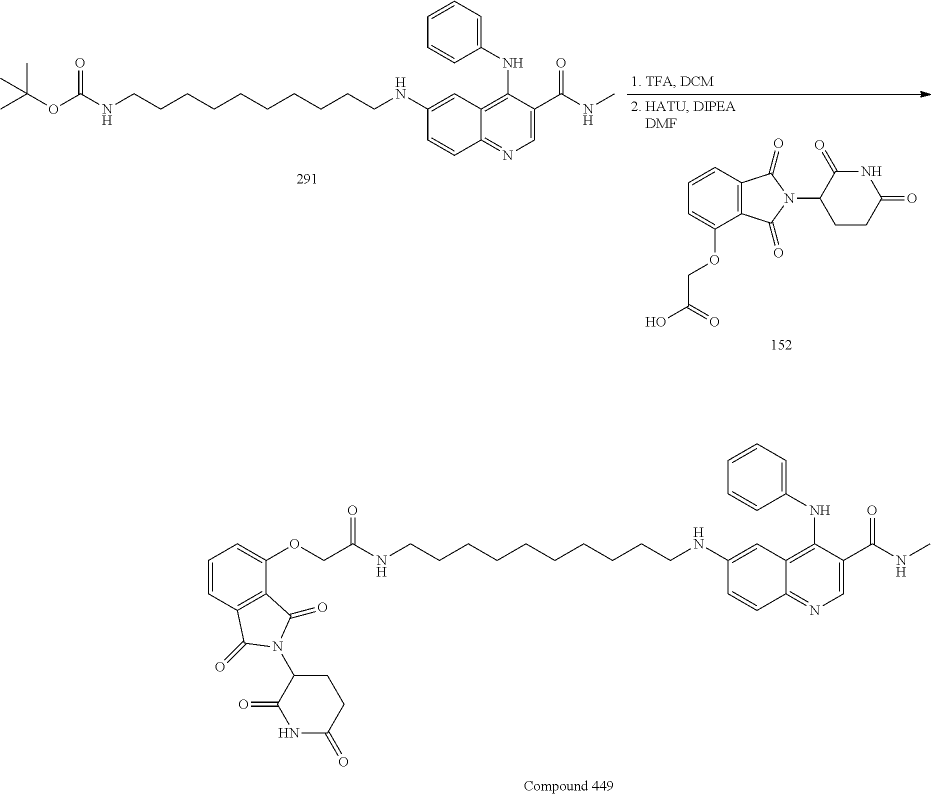 [SMILES: CNC(=O)c1cnc2ccc(NCCCCCCCCCCNC(=O)OC(C)(C)C)cc2c1Nc1ccccc1 ; O=C(O)COc1cccc2c1C(=O)N(C1CCC(=O)NC1=O)C2=O ; CNC(=O)c1cnc2ccc(NCCCCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of tert-butyl N-[10-[[4-anilino-3-(methylcarbamoyl)-6-quinolyl]amino]decyl]carbamate (291, 230 mg, 419.92 μmol) in anhydrous DCM (5 mL) was added TFA (239.39 mg, 2.10 mmol, 161.75 uL) under nitrogen atmosphere at room temperature, the resulting mixture was stirred at room temperature for 30 minutes. The reaction mixture was concentrated under reduced pressure to afford a crude residue which was dissolved in DMF (4 mL). DIPEA (162.81 mg, 1.26 mmol, 219.42 uL), 2-[2-(2,6-dioxo-3-piperidyl)-1,3-dioxo-isoindolin-4-yl]oxyacetic acid (152, 139.52 mg, 419.92 μmol) and HATU (239.50 mg, 629.87 μmol) were added under nitrogen atmosphere at room temperature. The resulting mixture was stirred at room temperature for 16 h. Ice cold water (15 mL) was added and stirred for 10 minutes, the resulting solid was filtered and dried to afford a crude solid which was purified by prep-HPLC (SUNFIRE OBD C18(100×30)MM 5μ) Mobile phase: A: 0.1% TFA in water B: ACN to afford 4-anilino-6-[10-[[2-[2-(2,6-dioxo-3-piperidyl)-1,3-dioxo-isoindolin-4-yl]oxyacetyl]amino]decylamino]-N-methyl-quinoline-3-carboxamide (Compound 449, 70 mg, 89.77 μmol, 21.38% yield) as a yellow solid. ¹H NMR (400 MHz, DMSO-d₆) δ 11.12 (s, 1H), 9.81 (s, 1H), 8.58 (d, J=10.0 Hz, 2H), 7.93 (t, J=5.7 Hz, 1H), 7.80 (dd, J=8.5, 7.3 Hz, 1H), 7.64 (d, J=9.1 Hz, 1H), 7.49 (d, J=7.3 Hz, 1H), 7.39 (d, J=8.6 Hz, 1H), 7.20 (t, J=7.9 Hz, 2H), 7.12 (dd, J=9.1, 2.5 Hz, 1H), 6.92 (t, J=7.4 Hz, 1H), 6.83 (d, J=8.0 Hz, 2H), 6.38 (d, J=2.5 Hz, 1H), 6.10 (t, J=5.3 Hz, 1H), 5.12 (dd, J=13.0, 5.3 Hz, 1H), 4.77 (s, 2H), 3.14 (q, J=6.7 Hz, 2H), 2.97-2.82 (m, 1H), 2.70 (d, J=4.5 Hz, 3H), 2.68-2.56 (m, 4H), 2.04 (dd, J=14.8, 9.2 Hz, 1H), 1.50-1.38 (m, 2H), 1.35-1.10 (m, 14H). LCMS (ES+): m/z 762 [M+H]⁺

### Synthesis of Compound 450

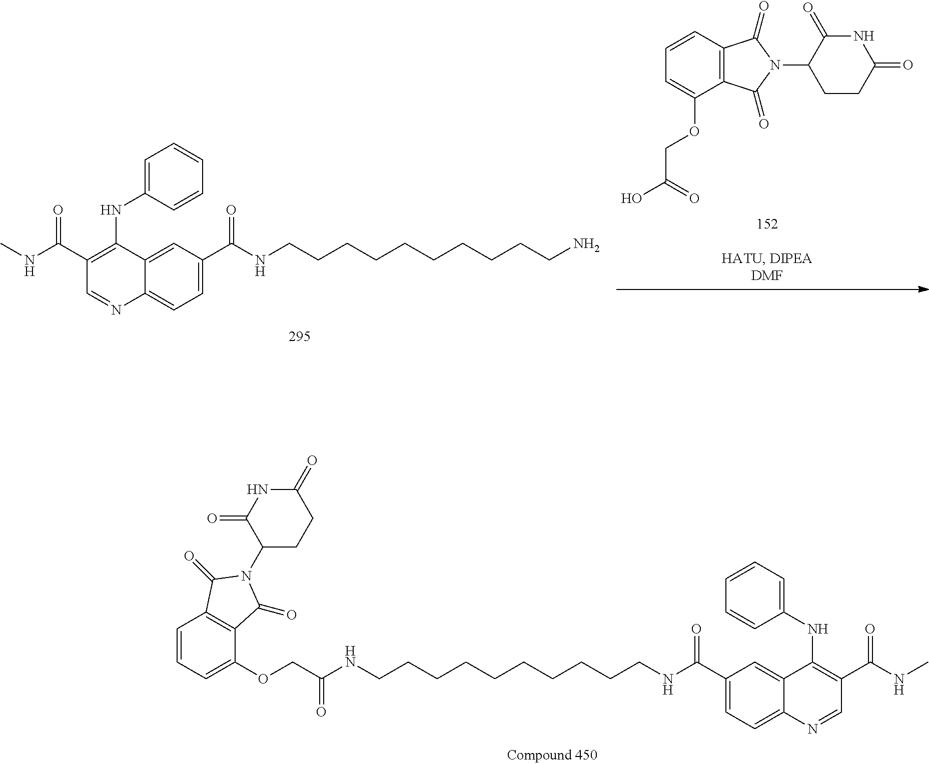 [SMILES: CNC(=O)c1cnc2ccc(C(=O)NCCCCCCCCCCN)cc2c1Nc1ccccc1 ; O=C(O)COc1cccc2c1C(=O)N(C1CCC(=O)NC1=O)C2=O ; CNC(=O)c1cnc2ccc(C(=O)NCCCCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of N6-(10-aminodecyl)-4-anilino-N3-methyl-quinoline-3,6-dicarboxamide (295, 60 mg, 126.15 μmol) in anhydrous DMF (2 mL) were added DIPEA (48.91 mg, 378.45 μmol, 65.92 uL), 2-[2-(2,6-dioxo-3-piperidyl)-1,3-dioxo-isoindolin-4-yl]oxyacetic acid (152, 41.92 mg, 126.15 μmol) and HATU (71.95 mg, 189.22 μmol) under nitrogen atmosphere at room temperature. The resulting mixture was stirred at room temperature for 16 h. The reaction mixture was concentrated under reduced pressure to afford a crude residue, which was purified by prep-HPLC (SUNFIRE OBD C18(100×30)MM 5μ) Mobile phase: A: 0.1% TFA in water B: ACN to afford 4-anilino-N6-[10-[[2-[2-(2,6-dioxo-3-piperidyl)-1,3-dioxo-isoindolin-4-yl]oxyacetyl]amino]decyl]-N3-methyl-quinoline-3,6-dicarboxamide (Compound 450, 6 mg, 7.36 μmol, 5.83% yield) as a yellow solid. LCMS (ES+): m/z 790 [M+H]⁺

### Synthesis of Compound 451

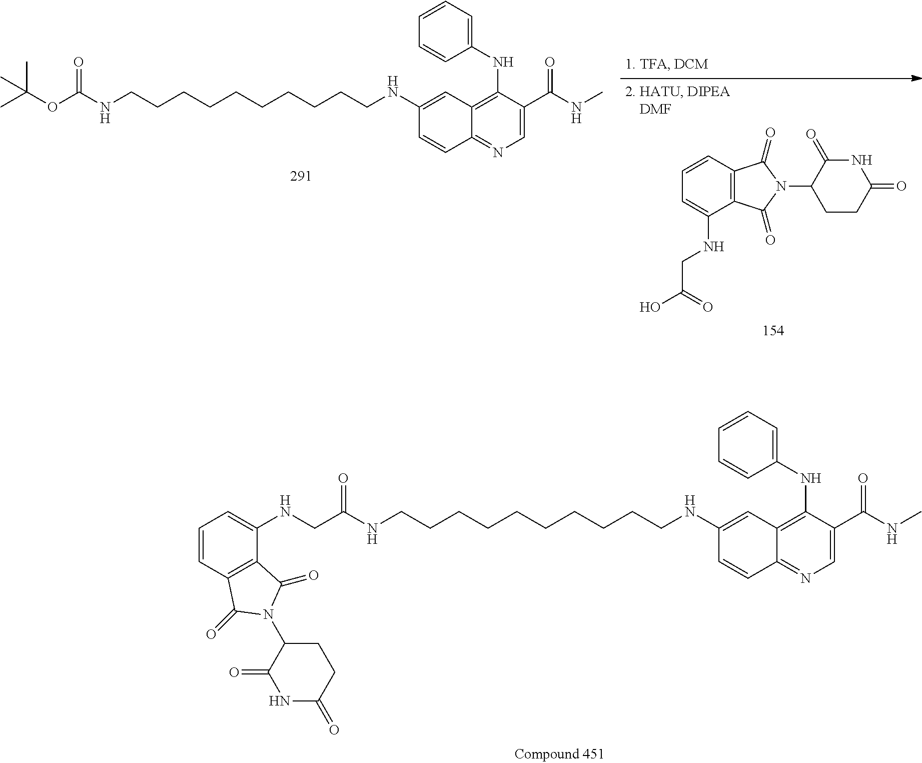 [SMILES: CNC(=O)c1cnc2ccc(NCCCCCCCCCCNC(=O)OC(C)(C)C)cc2c1Nc1ccccc1 ; O=C(O)CNc1cccc2c1C(=O)N(C1CCC(=O)NC1=O)C2=O ; CNC(=O)c1cnc2ccc(NCCCCCCCCCCNC(=O)CNc3cccc4c3C(=O)N(C3CCC(=O)NC3=O)C4=O)cc2c1Nc1ccccc1]

Into a 25 mL single-necked round-bottomed flask containing a well-stirred solution of tert-butyl N-[10-[[4-anilino-3-(methylcarbamoyl)-6-quinolyl]amino]decyl]carbamate (291, 75 mg, 136.93 μmol) in anhydrous DCM (4 mL) was added TFA (78.06 mg, 684.64 μmol, 52.75 uL) under nitrogen atmosphere at room temperature. The resulting mixture was stirred at room temperature for 30 minutes. The reaction mixture was concentrated under reduced pressure to afford a crude residue which was dissolved in DMF (3 mL). DIPEA (53.09 mg, 410.79 μmol, 71.55 uL), 2-[[2-(2,6-dioxo-3-piperidyl)-1,3-dioxo-isoindolin-4-yl]amino]acetic acid (154, 45.36 mg, 136.93 μmol) and HATU (78.10 mg, 205.39 μmol) were added under nitrogen atmosphere at room temperature. The resulting mixture was stirred at room temperature for 16 h. Ice cold water (15 mL) was added and stirred for 10 minutes, the resulting solid was filtered and dried to afford a crude solid which was purified by prep-HPLC (SUNFIRE OBD C18(100×30)MM 50 Mobile phase: A: 0.1% TFA in water B: ACN to afford 4-anilino-6-[10-[[2-[[2-(2,6-dioxo-3-piperidyl)-1,3-dioxo-isoindolin-4-yl]amino]acetyl]amino]decylamino]-N-methyl-quinoline-3-carboxamide (Compound 451, 25 mg, 30.92 μmol, 22.58% yield) as a yellow solid. ¹H NMR (400 MHz, DMSO-d₆) δ 11.11 (s, 1H), 9.82 (s, 1H), 8.59 (d, J=5.1 Hz, 1H), 8.58-8.55 (m, 1H), 8.08 (t, J=5.7 Hz, 1H), 7.64 (d, J=9.1 Hz, 1H), 7.58 (t, J=8.0 Hz, 1H), 7.20 (t, J=7.6 Hz, 2H), 7.16-7.11 (m, 1H), 7.06 (d, J=6.9 Hz, 1H), 6.99-6.89 (m, 2H), 6.84 (t, J=7.1 Hz, 3H), 6.38 (s, 1H), 6.10 (t, J=5.3 Hz, 1H), 5.07 (dd, J=13.0, 5.4 Hz, 1H), 3.91 (d, J=5.5 Hz, 2H), 3.08 (q, J=6.7 Hz, 2H), 2.95-2.83 (m, 1H), 2.70 (d, J=4.4 Hz, 3H), 2.67 (s, 3H), 2.09-1.98 (m, 1H), 1.92-1.87 (m, 1H), 1.47-1.35 (m, 2H), 1.34-1.28 (m, 2H), 1.23 (s, 10H), 1.18-1.12 (m, 4H). LCMS (ES+): m/z 761 [M+H]⁺

### Synthesis of Compound 452

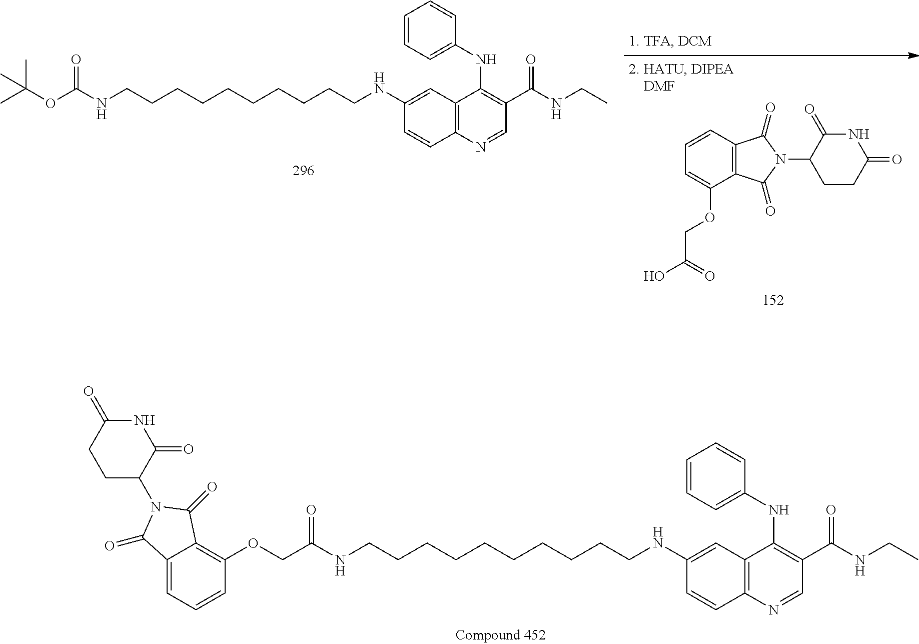 [SMILES: CCNC(=O)c1cnc2ccc(NCCCCCCCCCCNC(=O)OC(C)(C)C)cc2c1Nc1ccccc1 ; O=C(O)COc1cccc2c1C(=O)N(C1CCC(=O)NC1=O)C2=O ; CCNC(=O)c1cnc2ccc(NCCCCCCCCCCNC(=O)COc3cccc4c3C(=O)N(C3CCC(=O)NC3=O)C4=O)cc2c1Nc1ccccc1]

An oven dried round bottom flask was charged with a solution of tert-butyl N-[10-[[4-anilino-3-(ethylcarbamoyl)-6-quinolyl]amino]decyl]carbamate (296, 92.99 mg, 165.53 μmol) in Dichloromethane (3 mL), Trifluoroacetic acid (2.96 g, 25.96 mmol, 2 mL) was added at room temperature. The reaction mixture was stirred for 2 h at room temperature and the reaction mixture was concentrated under reduced pressure. To a solution of crude product in DMF (2 mL), 2-[2-(2,6-dioxo-3-piperidyl)-1,3-dioxo-isoindolin-4-yl]oxyacetic acid (152, 50 mg, 150.48 μmol), DIPEA (97.24 mg, 752.41 μmol, 131.05 uL) and HATU (68.66 mg, 180.58 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[10-[[2-[2-(2,6-dioxo-3-piperidyl)-1,3-dioxo-isoindolin-4-yl]oxyacetyl]amino]decylamino]-N-ethyl-quinoline-3-carboxamide (Compound 452, 4.79 mg, 6.13 μmol, 4.07% yield) as yellow solid. LCMS (ES+): m/z 776 [M+H]⁺

### Synthesis of Compound 453

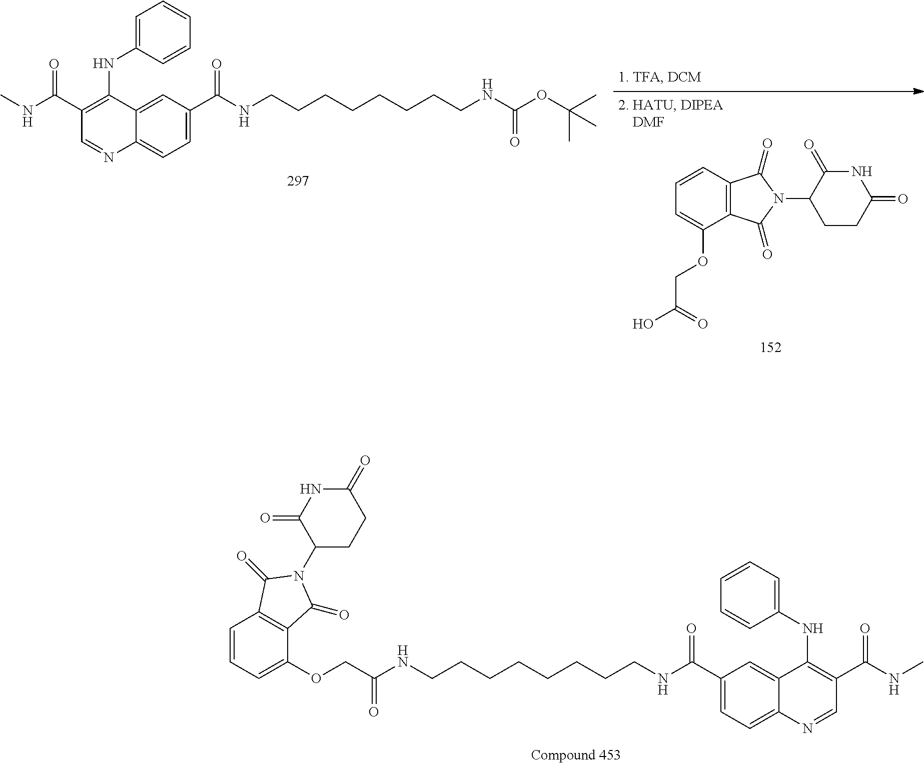 [SMILES: CNC(=O)c1cnc2ccc(C(=O)NCCCCCCCCNC(=O)OC(C)(C)C)cc2c1Nc1ccccc1 ; O=C(O)COc1cccc2c1C(=O)N(C1CCC(=O)NC1=O)C2=O ; CNC(=O)c1cnc2ccc(C(=O)NCCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of tert-butyl N-[8-[[4-anilino-3-(methylcarbamoyl)quinoline-6-carbonyl]amino]octyl]carbamate (297, 100 mg, 182.59 μmol) in anhydrous DCM (4 mL) was added TFA (104.09 mg, 912.93 μmol, 70.33 uL) under nitrogen atmosphere at room temperature, the resulting mixture was stirred at room temperature for 30 minutes. The reaction mixture was concentrated under reduced pressure to afford a crude residue which was dissolved in DMF (3 mL) were added DIPEA (70.79 mg, 547.76 μmol, 95.41 uL), 2-[2-(2,6-dioxo-3-piperidyl)-1,3-dioxo-isoindolin-4-yl]oxyacetic acid (152, 66.73 mg, 200.84 μmol) and HATU (104.14 mg, 273.88 μmol) under nitrogen atmosphere at room temperature. The resulting mixture was stirred at room temperature for 16 h. Ice cold water (15 mL) was added and stirred for 10 minutes, the resulting solid was filtered and dried to afford a crude solid which was purified by prep-HPLC (SUNFIRE OBD C18(100×30)MM 50 Mobile phase: A: 0.1% TFA in water B: ACN to afford 4-anilino-N6-[8-[[2-[2-(2,6-dioxo-3-piperidyl)-1,3-dioxo-isoindolin-4-yl]oxyacetyl]amino]octyl]-N3-methyl-quinoline-3,6-dicarboxamide (Compound 453, 35 mg, 45.25 μmol, 24.78% yield) as a yellow solid. ¹H NMR (400 MHz, DMSO-d₆) δ 11.11 (s, 1H), 8.94 (s, 1H), 8.81 (s, 1H), 8.66-8.58 (m, 1H), 8.47-8.42 (m, 1H), 8.29 (d, J=8.8 Hz, 1H), 8.00 (d, J=8.8 Hz, 1H), 7.93 (t, J=5.7 Hz, 1H), 7.80 (t, J=7.9 Hz, 1H), 7.49 (d, J=7.2 Hz, 1H), 7.42-7.35 (m, 3H), 7.28-7.18 (m, 3H), 5.11 (dd, J=13.0, 5.4 Hz, 1H), 4.76 (s, 2H), 3.28 (t, J=6.5 Hz, 2H), 3.13 (d, J=6.5 Hz, 2H), 2.97-2.83 (m, 1H), 2.68-2.56 (m, 2H), 2.34 (d, J=4.7 Hz, 3H), 2.02 (s, 1H), 1.58-1.48 (m, 2H), 1.48-1.38 (m, 2H), 1.27 (s, 8H). LCMS (ES+): m/z 762 [M+H]⁺

### Synthesis of Compound 454

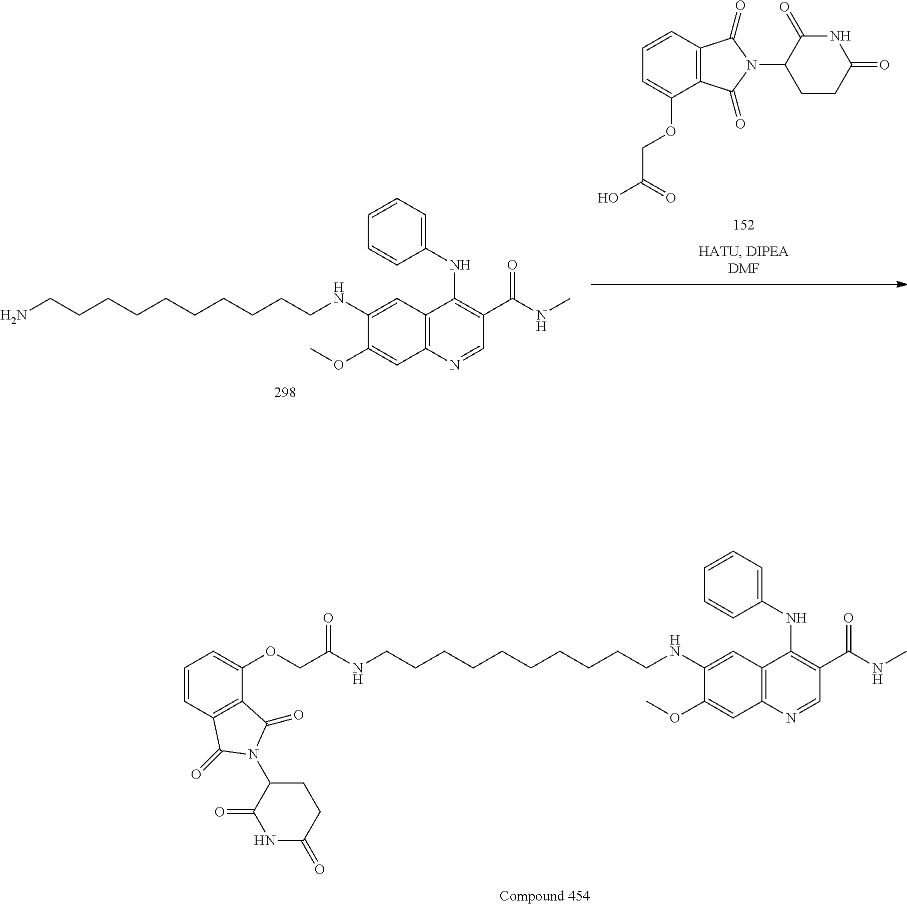 [SMILES: O=C(O)COc1cccc2c1C(=O)N(C1CCC(=O)NC1=O)C2=O ; CNC(=O)c1cnc2cc(OC)c(NCCCCCCCCCCN)cc2c1Nc1ccccc1 ; CNC(=O)c1cnc2cc(OC)c(NCCCCCCCCCCNC(=O)COc3cccc4c3C(=O)N(C3CCC(=O)NC3=O)C4=O)cc2c1Nc1ccccc1]

To a stirred solution of 2-[2-(2,6-dioxo-3-piperidyl)-1,3-dioxo-isoindolin-4-yl]oxyacetic acid (152, 83.48 mg, 251.24 μmol), 6-(10-aminodecylamino)-4-anilino-7-methoxy-N-methyl-quinoline-3-carboxamide (298, 100 mg, 209.36 μmol) in DMF (5 mL) was added DIPEA (135.29 mg, 1.05 mmol, 182.34 uL) and HATU (119.41 mg, 314.04 μmol). The reaction mixture was stirred for 16 hr at 25° C. The reaction mixture was diluted with water (10 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[10-[[2-[2-(2,6-dioxo-3-piperidyl)-1,3-dioxo-isoindolin-4-yl]oxyacetyl]amino]decylamino]-7-methoxy-N-methyl-quinoline-3-carboxamide (Compound 454, 30 mg, 36.75 μmol, 17.55% yield) as a yellow solid. ¹H NMR (400 MHz, DMSO-d₆) δ 11.12 (s, 1H), 10.88 (s, 1H), 8.60-8.53 (m, 2H), 7.93 (t, J=5.8 Hz, 1H), 7.80 (dd, J=8.5, 7.3 Hz, 1H), 7.49 (d, J=7.2 Hz, 1H), 7.42-7.36 (m, 2H), 7.24 (t, J=7.4 Hz, 1H), 7.19 (d, J=6.2 Hz, 2H), 6.80 (s, 1H), 6.01 (s, 1H), 5.11 (dd, J=12.9, 5.4 Hz, 1H), 4.76 (s, 2H), 4.01 (s, 3H), 3.13 (q, J=6.7 Hz, 2H), 2.92-2.84 (m, 3H), 2.67 (p, J=1.9 Hz, 2H), 2.62-2.56 (m, 2H), 2.33 (p, J=1.9 Hz, 2H), 2.07-1.99 (m, 2H), 1.47-1.35 (m, 4H), 1.28-1.18 (m, 10H). LCMS (ES+): m/z 792 [M+H]⁺

### Synthesis of Compound 455

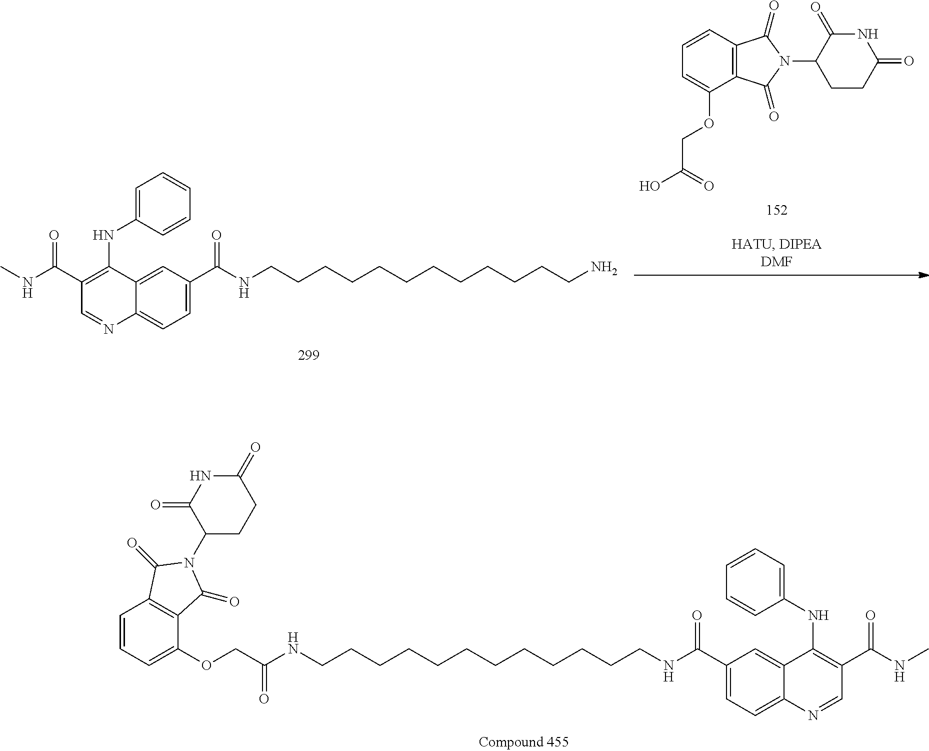 [SMILES: CNC(=O)c1cnc2ccc(C(=O)NCCCCCCCCCCCCN)cc2c1Nc1ccccc1 ; O=C(O)COc1cccc2c1C(=O)N(C1CCC(=O)NC1=O)C2=O ; CNC(=O)c1cnc2ccc(C(=O)NCCCCCCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of N6-(12-aminododecyl)-4-anilino-N3-methyl-quinoline-3,6-dicarboxamide (299, 29.20 mg, 57.97 μmol) in anhydrous DMF (2 mL) were added DIPEA (22.47 mg, 173.90 μmol, 30.29 uL), 2-[2-(2,6-dioxo-3-piperidyl)-1,3-dioxo-isoindolin-4-yl]oxyacetic acid (152, 19.26 mg, 57.97 μmol) and HATU (33.06 mg, 86.95 μmol) under nitrogen atmosphere at room temperature. The resulting mixture was stirred at room temperature for 16 h. The reaction mixture was concentrated under reduced pressure to afford a crude residue, which was purified by prep-HPLC (SUNFIRE OBD C18(100×30)MM 5μ) Mobile phase: A: 0.1% TFA in water B: ACN to afford 4-anilino-N6-[12-[[2-[2-(2,6-dioxo-3-piperidyl)-1,3-dioxo-isoindolin-4-yl]oxyacetyl]amino]dodecyl]-N3-methyl-quinoline-3,6-dicarboxamide (Compound 455, 19 mg, 22.47 μmol, 38.76% yield) as a yellow solid. ¹H NMR (400 MHz, DMSO-d₆) δ 11.12 (s, 1H), 8.94 (s, 1H), 8.81 (s, 1H), 8.62 (s, 1H), 8.47-8.41 (m, 1H), 8.28 (d, J=9.0 Hz, 1H), 8.00 (d, J=8.9 Hz, 1H), 7.92 (t, J=5.9 Hz, 1H), 7.80 (t, J=7.9 Hz, 1H), 7.49 (d, J=7.2 Hz, 1H), 7.37 (dd, J=10.1, 7.8 Hz, 3H), 7.21 (t, J=9.5 Hz, 3H), 5.12 (dd, J=13.2, 5.2 Hz, 1H), 4.76 (s, 2H), 3.31-3.25 (m, 2H), 3.18-3.08 (m, 2H), 2.94-2.84 (m, 2H), 2.68-2.56 (m, 3H), 2.33 (s, 3H), 2.08-2.00 (m, 1H), 1.57-1.47 (m, 2H), 1.47-1.38 (m, 2H), 1.31-1.21 (m, 14H). LCMS (ES+): m/z 818 [M+H]⁺

### Synthesis of Compound 456

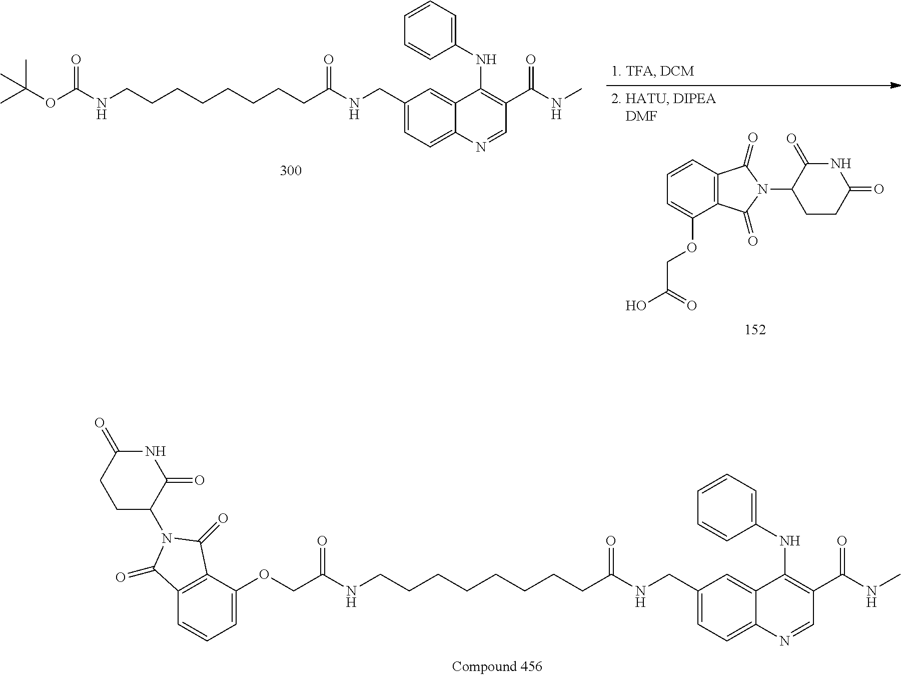 [SMILES: CNC(=O)c1cnc2ccc(CNC(=O)CCCCCCCCNC(=O)OC(C)(C)C)cc2c1Nc1ccccc1 ; O=C(O)COc1cccc2c1C(=O)N(C1CCC(=O)NC1=O)C2=O ; CNC(=O)c1cnc2ccc(CNC(=O)CCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of tert-butyl N-[9-[[4-anilino-3-(methylcarbamoyl)-6-quinolyl]methylamino]-9-oxo-nonyl]carbamate (300, 100 mg, 178.03 μmol) in anhydrous DCM (4 mL) was added TFA (101.49 mg, 890.13 μmol, 68.58 uL) under nitrogen atmosphere at room temperature. The resulting mixture was stirred at room temperature for 30 minutes. The reaction mixture was concentrated under reduced pressure to afford a crude residue which was dissolved in DMF (3 mL). DIPEA (69.02 mg, 534.08 μmol, 93.02 uL), 2-[2-(2,6-dioxo-3-piperidyl)-1,3-dioxo-isoindolin-4-yl]oxyacetic acid (152, 59.15 mg, 178.03 μmol) and HATU (101.54 mg, 267.04 μmol) were added. The resulting mixture was stirred at room temperature for 16 h. The reaction mixture was concentrated to afford crude residue which was purified by prep-HPLC (SUNFIRE OBD C18(100×30)MM 5μ) Mobile phase: A: 0.1% TFA in water B: ACN to afford 4-anilino-64-[9-[[2-[2-(2,6-dioxo-3-piperidyl)-1,3-dioxo-isoindolin-4-yl]oxyacetyl]amino]nonanoylamino]methyl]-N-methyl-quinoline-3-carboxamide (Compound 456, 10 mg, 11.47 μmol, 6.44% yield) as a yellow solid. ¹H NMR (400 MHz, DMSO-d₆) δ 11.12 (s, 1H), 8.80 (s, 1H), 8.57-8.51 (m, 1H), 8.24 (s, 1H), 7.96-7.89 (m, 2H), 7.84-7.77 (m, 1H), 7.65 (s, 1H), 7.49 (d, J=7.3 Hz, 1H), 7.39 (d, J=8.6 Hz, 1H), 7.25 (t, J=8.2 Hz, 2H), 7.03 (s, 1H), 6.97 (s, 1H), 5.12 (dd, J=13.1, 5.5 Hz, 1H), 4.77 (s, 2H), 4.29 (d, J=5.8 Hz, 2H), 3.18-3.10 (m, 2H), 2.95-2.83 (m, 2H), 2.00 (t, J=7.5 Hz, 4H), 1.43 (d, J=7.7 Hz, 4H), 1.23 (d, J=4.8 Hz, 12H). LCMS (ES+): m/z 776 [M+H]⁺

### Synthesis of Compound 457

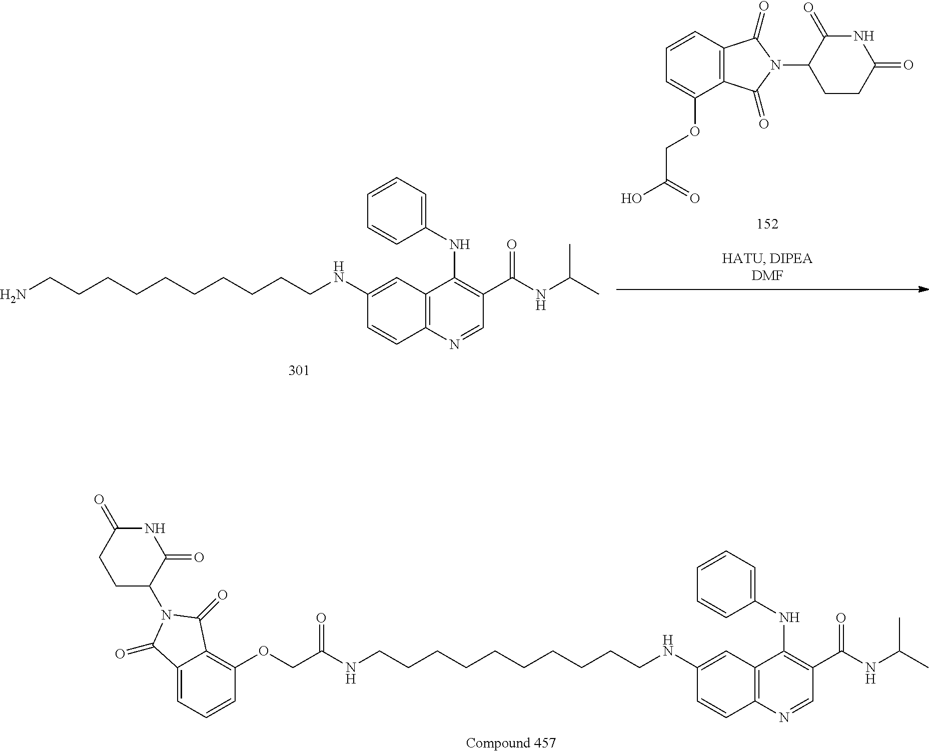 [SMILES: CC(C)NC(=O)c1cnc2ccc(NCCCCCCCCCCN)cc2c1Nc1ccccc1 ; O=C(O)COc1cccc2c1C(=O)N(C1CCC(=O)NC1=O)C2=O ; CC(C)NC(=O)c1cnc2ccc(NCCCCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of 6-(10-aminodecylamino)-4-anilino-N-isopropyl-quinoline-3-carboxamide (301, 71.58 mg, 150.48 μmol) and 2-[2-(2,6-dioxo-3-piperidyl)-1,3-dioxo-isoindolin-4-yl]oxyacetic acid (152, 50 mg, 150.48 μmol) in DMF (10 mL) were added DIPEA (58.35 mg, 451.45 μmol, 78.63 uL) and HATU (85.83 mg, 225.72 μmol) under nitrogen atmosphere at room temperature, the resulting mixture was stirred at room temperature for 16 h. After completion of reaction, 20 mL of cold water was added and stirred for 10 minutes. The resulting solid was filtered and dried to afford crude product, which was purified by reverse phase prep purification (SUNFIRE OBD C18(100×30)MM 50 Mobile phase: A: 0.1% TFA in water B: ACN) to obtain 4-anilino-6-[10-[[2-[2-(2,6-dioxo-3-piperidyl)-1,3-dioxo-isoindolin-4-yl]oxyacetyl]amino]decylamino]-N-isopropyl-quinoline-3-carboxamide (Compound 457, 35 mg, 44.31 μmol, 29.44% yield). ¹H NMR (400 MHz, DMSO-d₆) δ 14.28 (s, 1H), 11.12 (s, 1H), 10.68 (s, 1H), 8.50 (s, 1H), 8.35 (d, J=7.2 Hz, 1H), 7.92 (t, J=5.8 Hz, 1H), 7.80 (t, J=8.0 Hz, 1H), 7.73 (d, J=9.1 Hz, 1H), 7.49 (d, J=7.3 Hz, 1H), 7.43-7.35 (m, 4H), 7.25-7.17 (m, 2H), 6.92 (s, 1H), 6.63 (s, 1H), 5.11 (dd, J=12.9, 5.5 Hz, 1H), 4.76 (s, 2H), 3.63-3.53 (m, 1H), 3.13 (q, J=6.6 Hz, 2H), 2.89 (s, 3H), 2.69-2.57 (m, 2H), 2.33 (s, 1H), 2.12-1.99 (m, 3H), 1.52-1.38 (m, 4H), 1.25 (s, 12H), 0.96 (d, J=6.5 Hz, 6H). LCMS (ES+): m/z 790 [M+H]⁺

### Synthesis of Compound 458

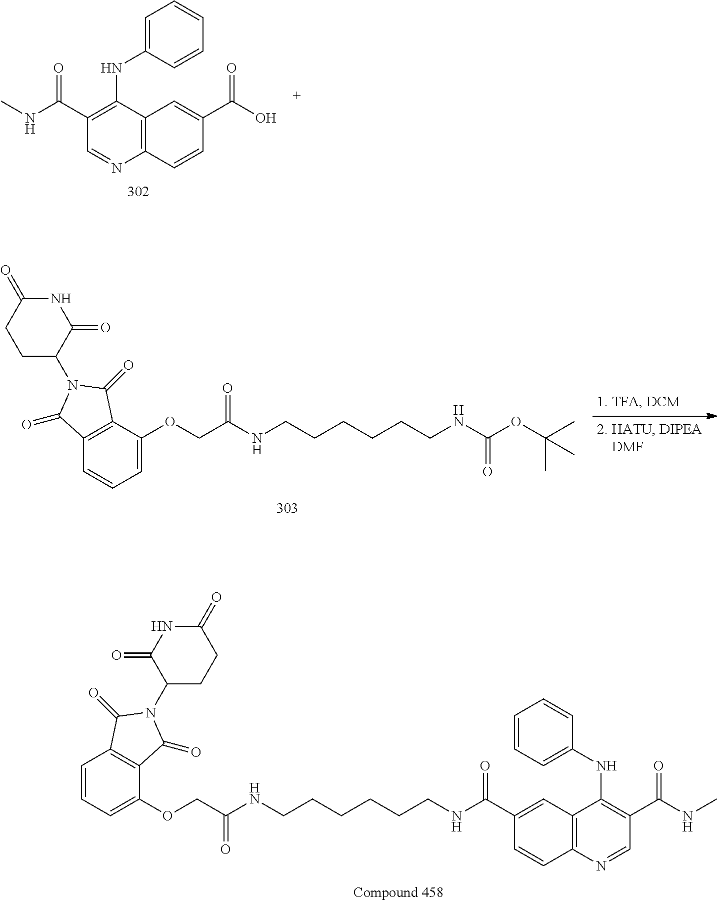 [SMILES: CNC(=O)c1cnc2ccc(C(=O)O)cc2c1Nc1ccccc1 ; CC(C)(C)OC(=O)NCCCCCCNC(=O)COc1cccc2c1C(=O)N(C1CCC(=O)NC1=O)C2=O ; CNC(=O)c1cnc2ccc(C(=O)NCCCCCCNC(=O)COc3cccc4c3C(=O)N(C3CCC(=O)NC3=O)C4=O)cc2c1Nc1ccccc1]

An oven dried round bottom flask was charged with a solution of tert-butyl N-[6-[[2-[2-(2,6-dioxo-3-piperidyl)-1,3-dioxo-isoindolin-4-yl]oxyacetyl]amino]hexyl]carbamate (303, 66.05 mg, 124.48 μmol) in Dichloromethane (3 mL), Trifluoroacetic acid (2.96 g, 25.96 mmol, 2 mL) was added at room temperature. The reaction mixture was stirred for 2 h at room temperature and the reaction mixture was concentrated under reduced pressure. To a solution of crude product in Dichloromethane (3 mL), 4-anilino-3-(methylcarbamoyl) quinoline-6-carboxylic acid (302, 40 mg, 124.48 μmol), DIPEA (80.44 mg, 622.41 μmol, 108.41 uL) and HATU (56.80 mg, 149.38 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-N6-[6-[[2-[2-(2,6-dioxo-3-piperidyl)-1,3-dioxo-isoindolin-4-yl]oxyacetyl]amino]hexyl]-N3-methyl-quinoline-3,6-dicarboxamide (Compound 458, 10.32 mg, 13.58 μmol, 10.91% yield) as yellow solid. ¹H NMR (400 MHz, DMSO-d₆) δ 11.12 (s, 1H), 8.95 (s, 1H), 8.82 (s, 1H), 8.67-8.59 (m, 1H), 8.48-8.40 (m, 1H), 8.33-8.25 (m, 1H), 7.99 (d, J=8.7 Hz, 1H), 7.97-7.92 (m, 1H), 7.80 (t, J=7.9 Hz, 1H), 7.48 (d, J=7.3 Hz, 1H), 7.43-7.34 (m, 2H), 7.29-7.17 (m, 2H), 5.11 (dd, J=12.9, 5.4 Hz, 1H), 4.76 (s, 2H), 3.31-3.24 (m, 2H), 3.20-3.11 (m, 2H), 2.92-2.82 (m, 1H), 2.67 (s, 3H), 2.58 (d, J=18.2 Hz, 2H), 2.33 (s, 3H), 2.03 (d, J=11.8 Hz, 2H), 1.57-1.41 (m, 4H). LCMS (ES+): m/z 734 [M+H]⁺

### Synthesis of Compound 459

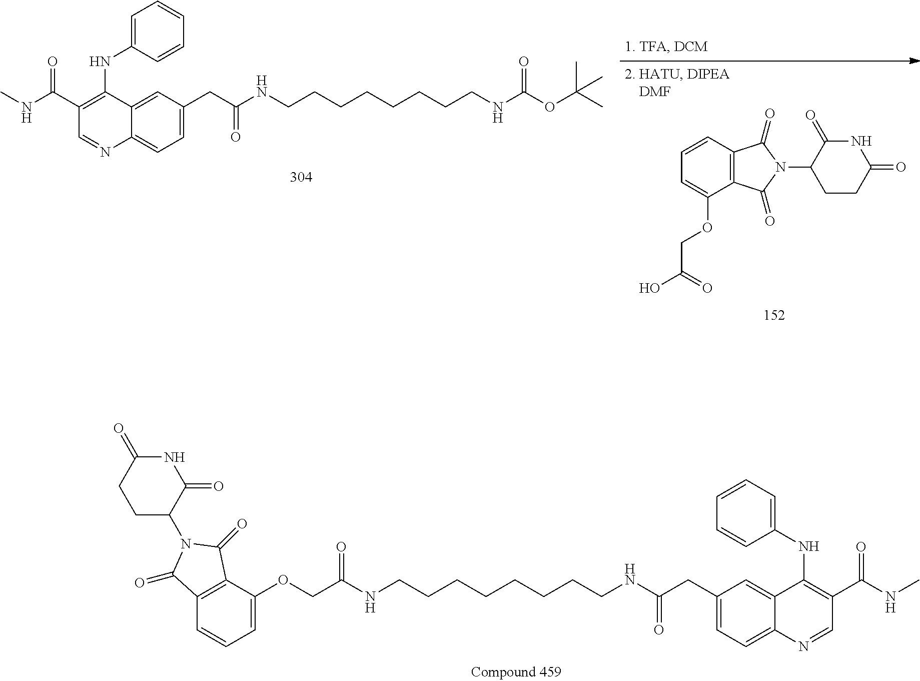 [SMILES: CNC(=O)c1cnc2ccc(CC(=O)NCCCCCCCCNC(=O)OC(C)(C)C)cc2c1Nc1ccccc1 ; O=C(O)COc1cccc2c1C(=O)N(C1CCC(=O)NC1=O)C2=O ; CNC(=O)c1cnc2ccc(CC(=O)NCCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of tert-butyl N-[8-[[2-[4-anilino-3-(methylcarbamoyl)-6-quinolyl]acetyl]amino]octyl]carbamate (304, 150 mg, 267.04 μmol) in anhydrous DCM (5 mL) was added TFA (152.24 mg, 1.34 mmol, 102.87 uL) under nitrogen atmosphere at room temperature, the resulting mixture was stirred at room temperature for 30 minutes. The reaction mixture was concentrated under reduced pressure to afford a crude residue which was dissolved in DMF (4 mL). DIPEA (103.54 mg, 801.12 μmol, 139.54 uL), 2-[2-(2,6-dioxo-3-piperidyl)-1,3-dioxo-isoindolin-4-yl]oxyacetic acid (152, 88.73 mg, 267.04 μmol) and HATU (152.31 mg, 400.56 μmol) were added under nitrogen atmosphere at room temperature. The resulting mixture was stirred at room temperature for 16 h. Ice cold water (15 mL) was added and stirred for 10 minutes, the resulting solid was filtered and dried to afford a crude solid which was purified by prep-HPLC (SUNFIRE OBD C18(100×30)MM 50 Mobile phase: A: 0.1% TFA in water B: ACN to afford 4-anilino-6-[2-[8-[[2-[2-(2,6-dioxo-3-piperidyl)-1,3-dioxo-isoindolin-4-yl]oxyacetyl]amino]octylamino]-2-oxo-ethyl]-N-methyl-quinoline-3-carboxamide (Compound 459, 22 mg, 28.36 μmol, 10.62% yield) as a yellow solid. ¹H NMR (400 MHz, DMSO-d₆) δ 11.12 (s, 1H), 10.99 (s, 1H), 8.76 (s, 1H), 8.42 (d, J=4.7 Hz, 1H), 8.33 (s, 1H), 8.10 (t, J=5.7 Hz, 1H), 7.96-7.91 (m, 2H), 7.88 (d, J=8.8 Hz, 1H), 7.80 (dd, J=8.5, 7.3 Hz, 1H), 7.49 (d, J=7.3 Hz, 1H), 7.38 (t, J=7.7 Hz, 3H), 7.25 (t, J=7.5 Hz, 1H), 7.19 (d, J=7.9 Hz, 2H), 5.11 (dd, J=12.9, 5.4 Hz, 1H), 4.76 (s, 2H), 3.57 (s, 2H), 3.12 (q, J=6.6 Hz, 2H), 3.02 (q, J=6.6 Hz, 2H), 2.95-2.83 (m, 2H), 2.67 (p, J=1.9 Hz, 1H), 2.63-2.53 (m, 2H), 2.34-2.28 (m, 4H), 2.08-1.97 (m, 1H), 1.45-1.32 (m, 4H), 1.22 (s, 8H). LCMS (ES+): m/z 776 [M+H]⁺

### Synthesis of Compound 460

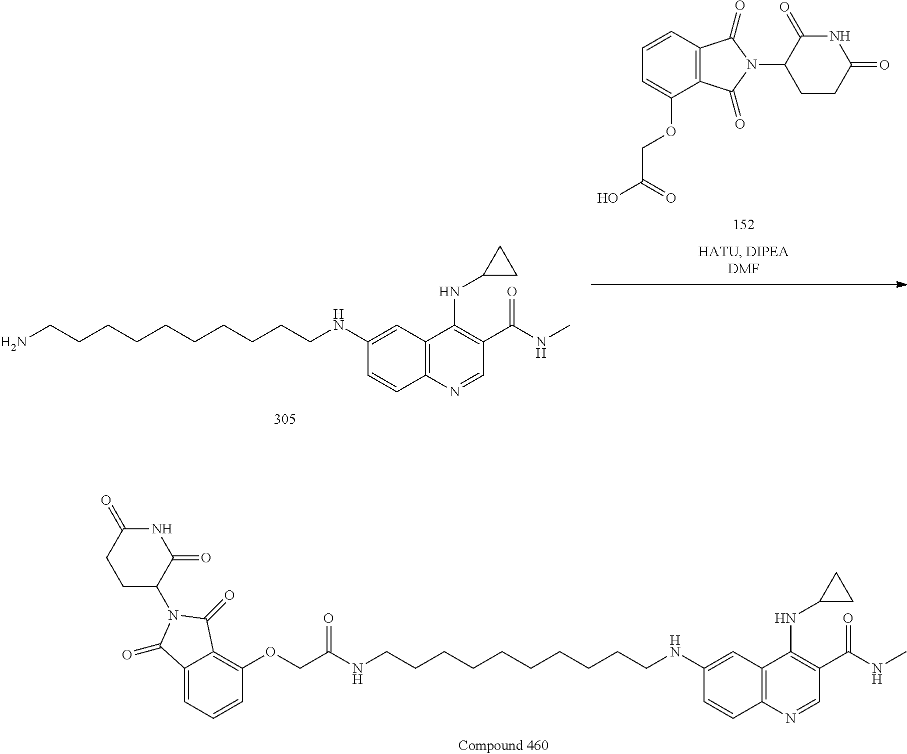 [SMILES: O=C(O)COc1cccc2c1C(=O)N(C1CCC(=O)NC1=O)C2=O ; CNC(=O)c1cnc2ccc(NCCCCCCCCCCN)cc2c1NC1CC1 ; CNC(=O)c1cnc2ccc(NCCCCCCCCCCNC(=O)COc3cccc4c3C(=O)N(C3CCC(=O)NC3=O)C4=O)cc2c1NC1CC1]

To a stirred solution of 2-[2-(2,6-dioxo-3-piperidyl)-1,3-dioxo-isoindolin-4-yl]oxyacetic acid (152, 80 mg, 240.77 μmol) and 6-(10-aminodecylamino)-4-(cyclopropylamino)-N-methyl-quinoline-3-carboxamide (305, 99.10 mg, 240.77 μmol) in DMF (10.0 mL) was added DIPEA (155.59 mg, 1.20 mmol, 209.69 uL) and HATU (137.32 mg, 361.16 μmol). The resulting mixture was stirred for 16 hr at 25° C. The resulting mixture was diluted with water (20 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-(cyclopropylamino)-6-[10-[[2-[2-(2,6-dioxo-3-piperidyl)-1,3-dioxo-isoindolin-4-yl]oxyacetyl]amino]decylamino]-N-methyl-quinoline-3-carboxamide (Compound 460, 50 mg, 66.07 μmol, 27.44% yield) as an yellow solid. ¹H NMR (400 MHz, DMSO-d₆) δ 13.85 (s, 1H), 11.10 (s, 1H), 8.58 (s, 1H), 8.39 (s, 1H), 7.90 (t, J=5.7 Hz, 1H), 7.84-7.78 (m, 1H), 7.62 (d, J=9.0 Hz, 1H), 7.49 (d, J=7.3 Hz, 1H), 7.40 (d, J=8.5 Hz, 1H), 7.38-7.33 (m, 2H), 6.51 (s, 1H), 5.11 (dd, J=13.0, 5.4 Hz, 1H), 4.77 (d, J=4.7 Hz, 2H), 3.14 (q, J=6.5 Hz, 4H), 3.06 (s, 1H), 2.90 (ddd, J=18.9, 14.3, 5.5 Hz, 1H), 2.80 (d, J=4.5 Hz, 3H), 2.70-2.57 (m, 2H), 2.11-1.99 (m, 1H), 1.67-1.58 (m, 2H), 1.48-1.37 (m, 4H), 1.26 (s, 10H), 0.86 (s, 2H), 0.68 (s, 2H). LCMS (ES+): m/z 726 [M+H]⁺

### Synthesis of Compound 461

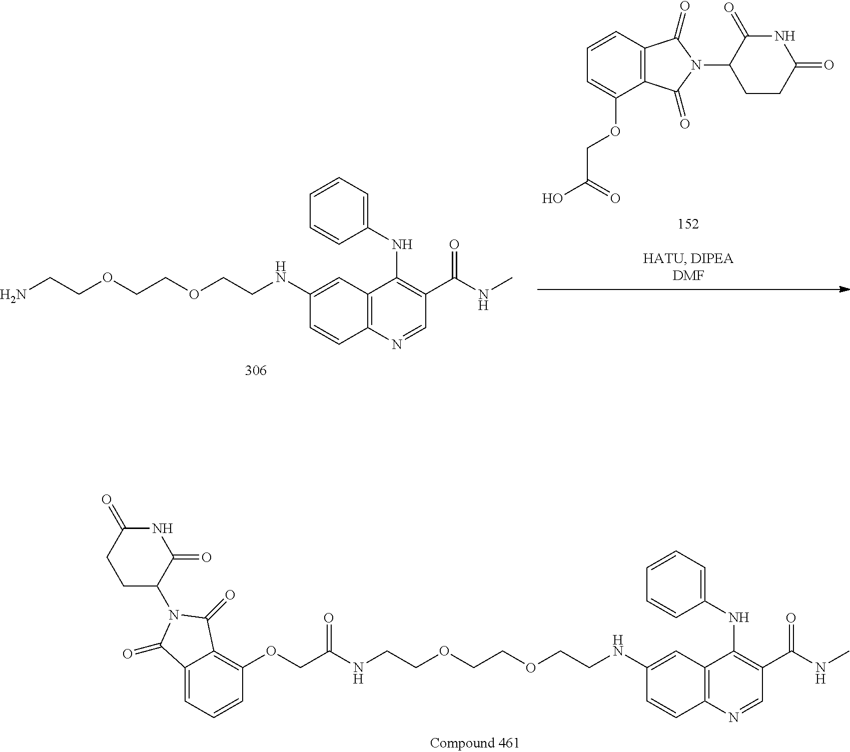 [SMILES: CNC(=O)c1cnc2ccc(NCCOCCOCCN)cc2c1Nc1ccccc1 ; O=C(O)COc1cccc2c1C(=O)N(C1CCC(=O)NC1=O)C2=O ; CNC(=O)c1cnc2ccc(NCCOCCOCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of 2-[2-(2,6-dioxo-3-piperidyl)-1,3-dioxo-isoindolin-4-yl]oxyacetic acid (152, 50 mg, 150.48 μmol) and 6-[2-[2-(2-aminoethoxy)ethoxy]ethylamino]-4-anilino-N-methyl-quinoline-3-carboxamide (306, 76.48 mg, 180.58 μmol) in DMF (10 mL) were added DIPEA (58.35 mg, 451.45 μmol, 78.63 uL) and HATU (85.83 mg, 225.72 μmol) under nitrogen atmosphere at room temperature, the resulting mixture was stirred at room temperature for 16 h. After completion of the reaction, 20 mL of cold water was added and stirred for 10 minutes. The resulting solid was filtered and dried to afford crude product, which was purified by reverse phase prep purification (SUNFIRE OBD C18(100×30)MM 5μ) Mobile phase: A: 0.1% TFA in water B: ACN) to obtain 4-anilino-6-[2-[2-[2-[[2-[2-(2,6-dioxo-3-piperidyl)-1,3-dioxo-isoindolin-4-yl]oxyacetyl]amino]ethoxy]ethoxy]ethylamino]-N-methyl-quinoline-3-carboxamide (Compound 461, 30 mg, 40.66 μmol, 27.02% yield). ¹H NMR (400 MHz, DMSO-d₆) δ 14.36 (s, 1H), 11.13 (s, 1H), 10.62 (s, 1H), 8.49 (s, 1H), 8.40 (q, J=4.5 Hz, 1H), 7.99 (t, J=5.6 Hz, 1H), 7.78 (dd, J=8.5, 7.3 Hz, 1H), 7.72 (d, J=9.2 Hz, 1H), 7.47 (s, 1H), 7.45-7.36 (m, 4H), 7.25 (t, J=7.4 Hz, 1H), 7.19 (d, J=7.8 Hz, 2H), 7.06 (d, J=2.3 Hz, 1H), 6.70 (s, 1H), 5.11 (dd, J=12.8, 5.5 Hz, 1H), 4.77 (s, 2H), 3.57-3.52 (m, 6H), 3.46 (t, J=5.7 Hz, 2H), 3.30 (q, J=5.7 Hz, 2H), 3.17 (t, J=5.6 Hz, 2H), 2.88 (ddd, J=17.2, 14.1, 5.3 Hz, 1H), 2.69-2.57 (m, 2H), 2.32 (d, J=4.6 Hz, 3H), 2.08-1.97 (m, 1H). LCMS (ES+): m/z 738 [M+H]⁺

### Synthesis of Compound 462

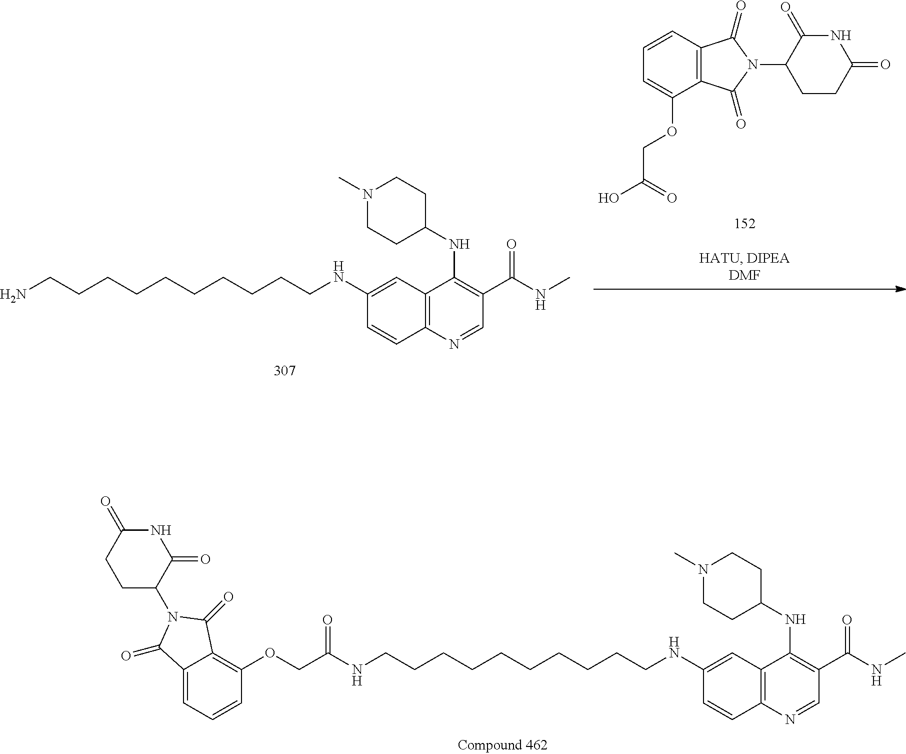 [SMILES: CNC(=O)c1cnc2ccc(NCCCCCCCCCCN)cc2c1NC1CCN(C)CC1 ; O=C(O)COc1cccc2c1C(=O)N(C1CCC(=O)NC1=O)C2=O ; CNC(=O)c1cnc2ccc(NCCCCCCCCCCNC(=O)COc3cccc4c3C(=O)N(C3CCC(=O)NC3=O)C4=O)cc2c1NC1CCN(C)CC1]

To a stirred solution of 2-[2-(2,6-dioxo-3-piperidyl)-1,3-dioxo-isoindolin-4-yl]oxyacetic acid (152, 160 mg, 481.54 μmol) and 6-(10-aminodecylamino)-N-methyl-4-[(1-methyl-4-piperidyl)amino]quinoline-3-carboxamide (307, 225.69 mg, 481.54 μmol) in DMF (10.0 mL) was added DIPEA (311.18 mg, 2.41 mmol, 419.38 uL) and HATU (274.65 mg, 722.32 μmol) and stirred for 16 hr at 25° C. The resulting mixture was diluted with water (30 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase prep HPLC to yield 6-[10-[[2-[2-(2,6-dioxo-3-piperidyl)-1,3-dioxo-isoindolin-4-yl]oxyacetyl]amino]decylamino]-N-methyl-4-[(1-methyl-4-piperidyl)amino]quinoline-3-carboxamide (Compound 462, 50 mg, 61.00 μmol, 12.67% yield) as an yellow solid. ¹H NMR (400 MHz, Methanol-d₄) δ 8.33 (s, 1H), 7.79 (dd, J=8.5, 7.3 Hz, 1H), 7.62 (d, J=9.1 Hz, 1H), 7.52 (d, J=7.3 Hz, 1H), 7.41 (d, J=8.5 Hz, 1H), 7.32 (dd, J=9.1, 2.4 Hz, 1H), 7.04 (d, J=2.4 Hz, 1H), 5.13 (dd, J=12.6, 5.4 Hz, 1H), 4.75 (s, 3H), 4.11 (s, 1H), 3.43 (d, J=12.2 Hz, 2H), 3.21 (t, J=7.0 Hz, 2H), 2.96 (s, 4H), 2.89-2.82 (m, 1H), 2.77 (d, J=6.5 Hz, 4H), 2.75-2.68 (m, 1H), 2.32-2.23 (m, 2H), 2.19-2.10 (m, 1H), 2.05-1.93 (m, 2H), 1.70 (p, J=7.0 Hz, 2H), 1.60-1.53 (m, 2H), 1.51-1.43 (m, 2H), 1.41-1.30 (m, 11H). LCMS (ES+): m/z 783 [M+H]⁺

### Synthesis of Compound 463

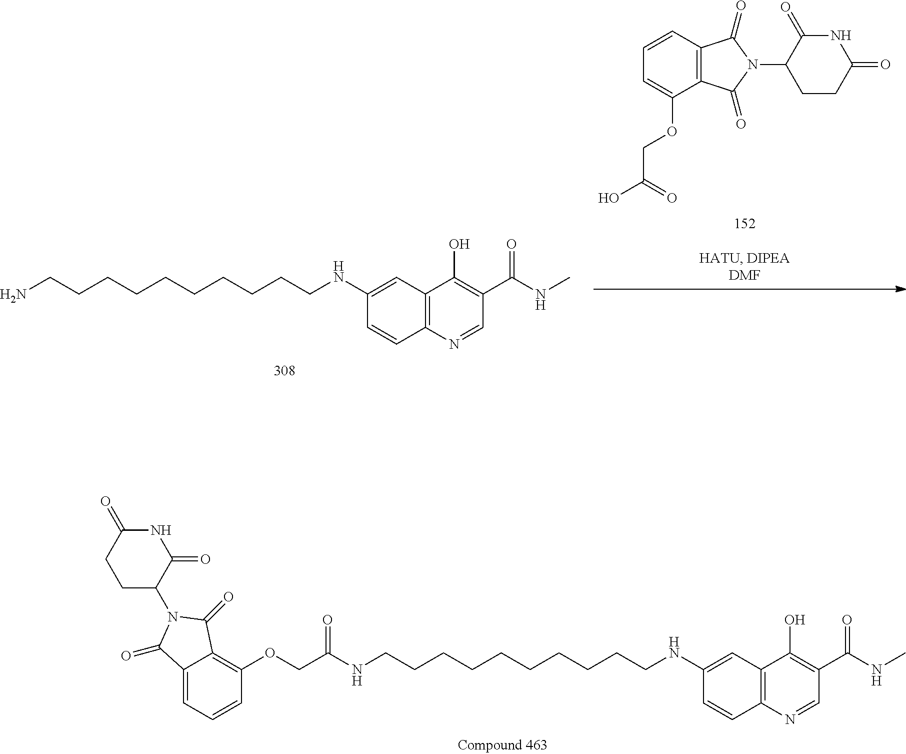 [SMILES: O=C(O)COc1cccc2c1C(=O)N(C1CCC(=O)NC1=O)C2=O ; CNC(=O)c1cnc2ccc(NCCCCCCCCCCN)cc2c1O ; CNC(=O)c1cnc2ccc(NCCCCCCCCCCNC(=O)COc3cccc4c3C(=O)N(C3CCC(=O)NC3=O)C4=O)cc2c1O]

An oven dried round bottom flask was charged with a solution of 2-[2-(2,6-dioxo-3-piperidyl)-1,3-dioxo-isoindolin-4-yl]oxyacetic acid (152, 70 mg, 210.68 μmol) in DMF (3 mL), 6-(10-aminodecylamino)-4-hydroxy-N-methyl-quinoline-3-carboxamide (308, 94.17 mg, 252.81 μmol), DIPEA (136.14 mg, 1.05 mmol, 183.48 uL) and HATU (96.13 mg, 252.81 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 6-[10-[[2-[2-(2,6-dioxo-3-piperidyl)-1,3-dioxo-isoindolin-4-yl]oxyacetyl]amino]decylamino]-4-hydroxy-N-methyl-quinoline-3-carboxamide (Compound 463, 3.98 mg, 5.03 μmol, 2.39% yield) as white solid. ¹H NMR (400 MHz, DMSO-d₆) δ 12.42 (d, J=6.9 Hz, 1H), 11.13 (s, 1H), 10.07 (d, J=5.1 Hz, 1H), 8.50 (d, J=6.8 Hz, 1H), 7.93 (t, J=5.7 Hz, 1H), 7.80 (t, J=7.9 Hz, 1H), 7.49 (d, J=7.3 Hz, 1H), 7.44 (d, J=8.9 Hz, 1H), 7.38 (d, J=8.5 Hz, 1H), 7.19-7.10 (m, 2H), 5.12 (dd, J=12.9, 5.4 Hz, 1H), 4.76 (s, 2H), 3.17-3.09 (m, 2H), 3.05 (t, J=7.0 Hz, 2H), 2.97-2.88 (m, 2H), 2.83 (d, J=4.7 Hz, 3H), 2.72-2.56 (m, 2H), 2.33 (q, J=1.9 Hz, 1H), 2.10-1.98 (m, 1H), 1.64-1.54 (m, 2H), 1.47-1.34 (m, 4H), 1.25 (s, 9H). LCMS (ES+): m/z 687 [M+H]⁺

### Synthesis of Compound 464

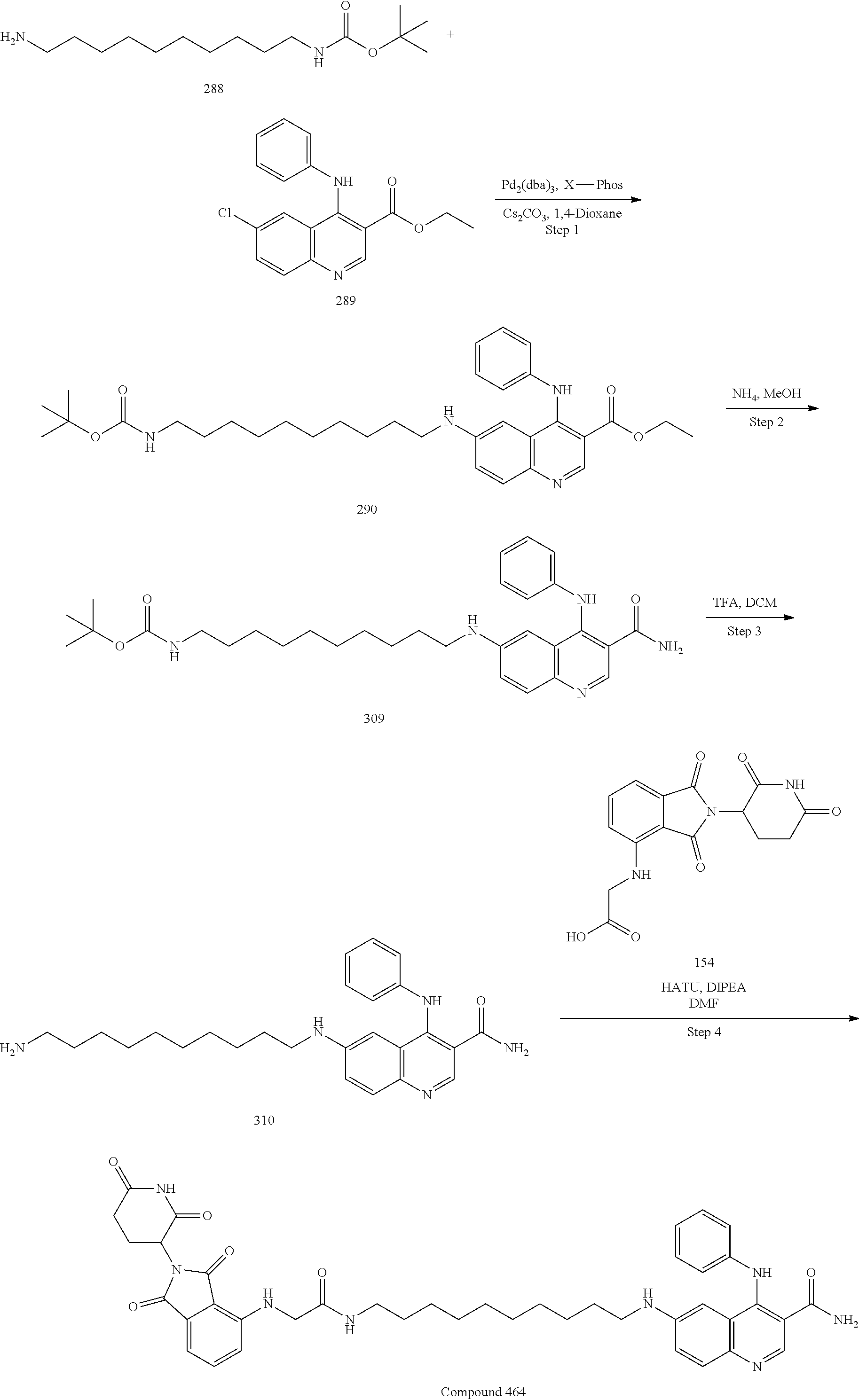

Step 1: An oven dried pressure tube was charged with a solution of ethyl 4-anilino-6-chloro-quinoline-3-carboxylate (289, 150 mg, 459.03 μmol) in 1,4-Dioxane (5 mL), tert-butyl N-(10-aminodecyl)carbamate (288, 137.56 mg, 504.93 μmol) and cesium carbonate (224.34 mg, 688.54 μmol) were added. The reaction mixture was purged with nitrogen for 5 minutes, 2-dicyclohexylphosphino-2′,4′,6′-triisopropylbiphenyl (43.77 mg, 91.81 μmol) and Pd₂(dba)₃ (42.03 mg, 45.90 μmol) were added. The reaction mixture was heated to 100° C. for 16 h and cooled to room temperature. The reaction mixture was diluted with water (5 mL) and the product was extracted with ethyl acetate (2×20 mL). The combined organic layer was dried over anhydrous sodium sulfate, filtered and concentrated under reduced pressure. The crude mixture was purified by column chromatography on silica (3% Methanol\Dichloromethane) to yield ethyl 4-anilino-6-[10-(tert-butoxycarbonylamino)decylamino]quinoline-3-carboxylate (290, 240 mg, 407.38 μmol, 88.75% yield) as yellow solid. LCMS (ES+): m/z 563 [M+H]⁺

Step 2: An oven dried pressure tube was charged with a solution of ethyl 4-anilino-6-[10-(tert-butoxycarbonylamino)decylamino]quinoline-3-carboxylate (290, 240 mg, 426.48 μmol) in ammonia solution (426.48 μmol, 10 mL) in Methanol (7N) and the reaction mixture was heated to 80° C. for 24 hr. The reaction mixture was cooled to room temperature and the reaction mixture was concentrated under reduced pressure. The crude mixture was purified by column chromatography on silica (8% Methanol\Dichloromethane) to yield tert-butyl N-[10-[(4-anilino-3-carbamoyl-6-quinolyl)amino]decyl]carbamate (309, 80 mg, 131.58 μmol, 30.85% yield) as yellow solid. LCMS (ES+): m/z 534 [M+H]⁺

Step 3: An oven dried round bottom flask was charged with a solution of tert-butyl N-[10-[(4-anilino-3-carbamoyl-6-quinolyl)amino]decyl]carbamate (309, 40 mg, 74.95 μmol) in Dichloromethane (3 mL), Trifluoroacetic acid (1.47 g, 12.89 mmol, 993.15 uL) was added at room temperature. The reaction mixture was stirred for an hour at room temperature and the reaction mixture was concentrated under reduced pressure to give crude 6-((10-aminodecyl)amino)-4-(phenylamino)quinoline-3-carboxamide (310).

Step 4: To a solution of crude 6-((10-aminodecyl)amino)-4-(phenylamino)quinoline-3-carboxamide (310) in DMF (2 mL), 2-[[2-(2,6-dioxo-3-piperidyl)-1,3-dioxo-isoindolin-4-yl]amino]acetic acid (154, 24.83 mg, 74.95 μmol), DIPEA (48.43 mg, 374.74 μmol, 65.27 uL) and HATU (34.20 mg, 89.94 μmol) were added. The reaction mixture was stirred for 16 h at room temperature and the reaction mixture was quenched with water (3 mL). The reaction mixture was concentrated under reduced pressure. The crude mixture was purified by reverse phase column chromatography (Column: SUNFIRE OBD C18(100×30)MM 5μ), Mobile phase: A: 0.1% TFA in water, B: ACN) to yield 4-anilino-6-[10-[[2-[[2-(2,6-dioxo-3-piperidyl)-1,3-dioxo-isoindolin-4-yl]amino]acetyl]amino]decylamino]quinoline-3-carboxamide (Compound 464, 6.36 mg, 7.85 μmol, 10.47% yield) as yellow solid. ¹H NMR (400 MHz, DMSO-d₆) δ 14.47 (s, 1H), 11.89 (s, 1H), 11.11 (s, 1H), 8.74 (s, 1H), 8.34 (s, 1H), 8.11-8.05 (m, 1H), 7.88 (s, 1H), 7.71 (d, J=9.1 Hz, 1H), 7.62-7.52 (m, 1H), 7.43 (t, J=7.7 Hz, 2H), 7.33 (dd, J=9.1, 2.3 Hz, 1H), 7.30-7.25 (m, 3H), 7.04 (dd, J=16.6, 7.1 Hz, 1H), 6.97-6.90 (m, 1H), 6.87-6.81 (m, 1H), 6.59 (s, 1H), 6.49 (d, J=2.3 Hz, 1H), 5.07 (dd, J=12.9, 5.4 Hz, 1H), 3.94-3.84 (m, 2H), 3.08 (q, J=6.5 Hz, 2H), 2.97-2.83 (m, 2H), 2.70-2.58 (m, 2H), 2.30-2.24 (m, 1H), 2.08-1.97 (m, 1H), 1.46-1.37 (m, 2H), 1.35-1.29 (m, 2H), 1.27-1.18 (m, 12H), 0.07 (s, 2H). LCMS (ES+): m/z 747 [M+H]⁺

### Synthesis of Compound 465

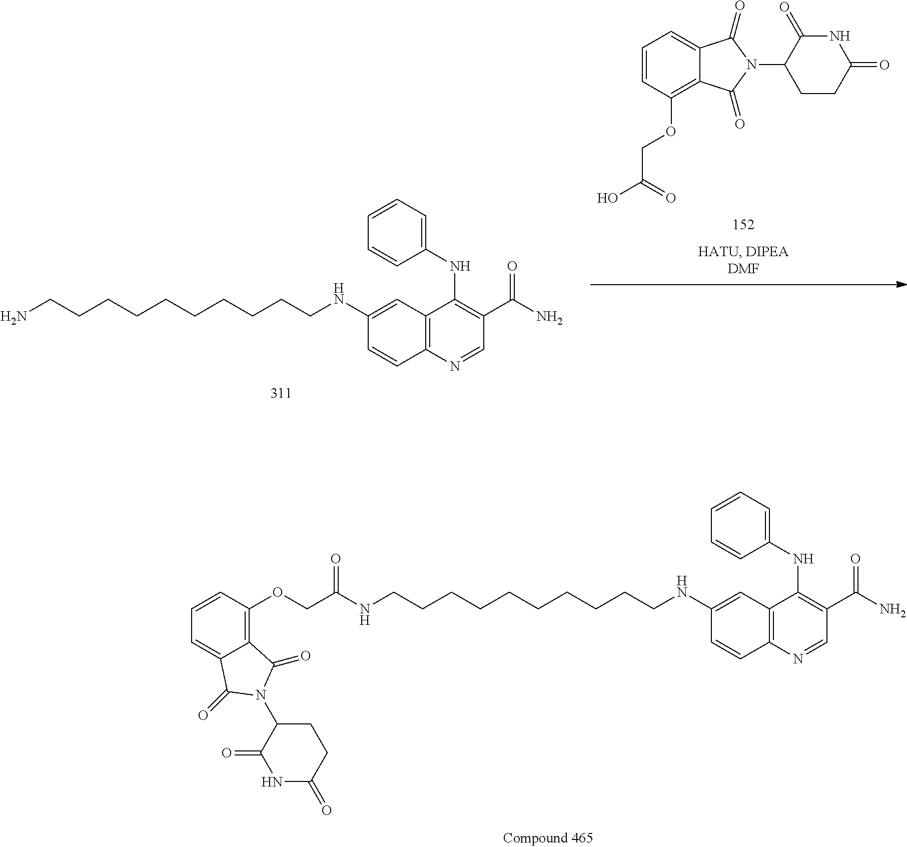 [SMILES: O=C(O)COc1cccc2c1C(=O)N(C1CCC(=O)NC1=O)C2=O ; NCCCCCCCCCCNc1ccc2ncc(C(N)=O)c(Nc3ccccc3)c2c1 ; NC(=O)c1cnc2ccc(NCCCCCCCCCCNC(=O)COc3cccc4c3C(=O)N(C3CCC(=O)NC3=O)C4=O)cc2c1Nc1ccccc1]

To a stirred solution of 2-[2-(2,6-dioxo-3-piperidyl)-1,3-dioxo-isoindolin-4-yl]oxyacetic acid (152, 93 mg, 279.90 μmol), 6-(10-aminodecylamino)-4-anilino-quinoline-3-carboxamide (311, 121.36 mg, 279.90 μmol) in DMF (10.0 mL) was added DIPEA (180.87 mg, 1.40 mmol, 243.77 uL) and HATU (159.64 mg, 419.85 μmol). The resulting solution was stirred for 16 hr at 25° C. The reaction mixture was diluted with water (20 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[10-[[2-[2-(2,6-dioxo-3-piperidyl)-1,3-dioxo-isoindolin-4-yl]oxyacetyl]amino]decylamino]quinoline-3-carboxamide (Compound 465, 130 mg, 166.78 μmol, 59.59% yield) as an yellow solid. LCMS (ES+): m/z 748 [M+H]⁺

### Synthesis of Compound 466

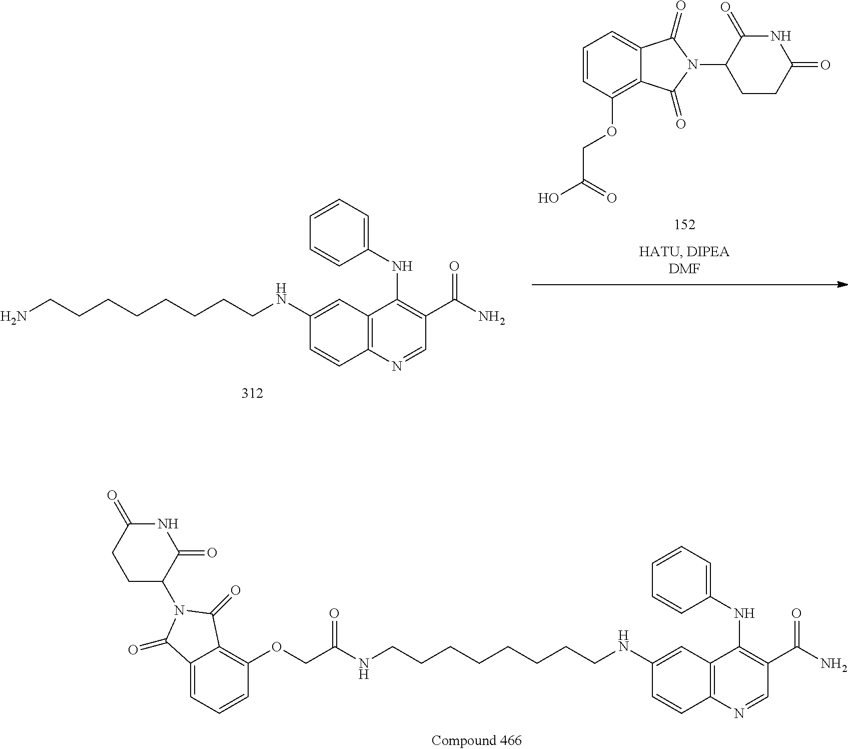 [SMILES: O=C(O)COc1cccc2c1C(=O)N(C1CCC(=O)NC1=O)C2=O ; NCCCCCCCCNc1ccc2ncc(C(N)=O)c(Nc3ccccc3)c2c1 ; NC(=O)c1cnc2ccc(NCCCCCCCCNC(=O)COc3cccc4c3C(=O)N(C3CCC(=O)NC3=O)C4=O)cc2c1Nc1ccccc1]

To a stirred solution of 2-[2-(2,6-dioxo-3-piperidyl)-1,3-dioxo-isoindolin-4-yl]oxyacetic acid (152, 85 mg, 255.82 μmol) and 6-(8-aminooctylamino)-4-anilino-quinoline-3-carboxamide (312, 103.74 mg, 255.82 μmol) in DMF (10.0 mL), was added DIPEA (165.31 mg, 1.28 mmol, 222.80 uL) and HATU (145.91 mg, 383.73 μmol) and stirred for 16 hr at 25° C. The reaction mixture was diluted with water (20 mL) and extracted with ethyl acetate (3×25 mL). The combined organic extracts were washed with water and brine solution, dried over anhydrous sodium sulfate, filtered and evaporated the solvent completely under reduced pressure. The resulting crude was purified by reverse phase preparative HPLC to yield 4-anilino-6-[8-[[2-[2-(2,6-dioxo-3-piperidyl)-1,3-dioxo-isoindolin-4-yl]oxyacetyl]amino]octylamino]quinoline-3-carboxamide (Compound 466, 110 mg, 147.90 μmol, 57.82% yield) as an yellow solid. LCMS (ES+): m/z 720 [M+H]⁺

### Synthesis of Compound 467

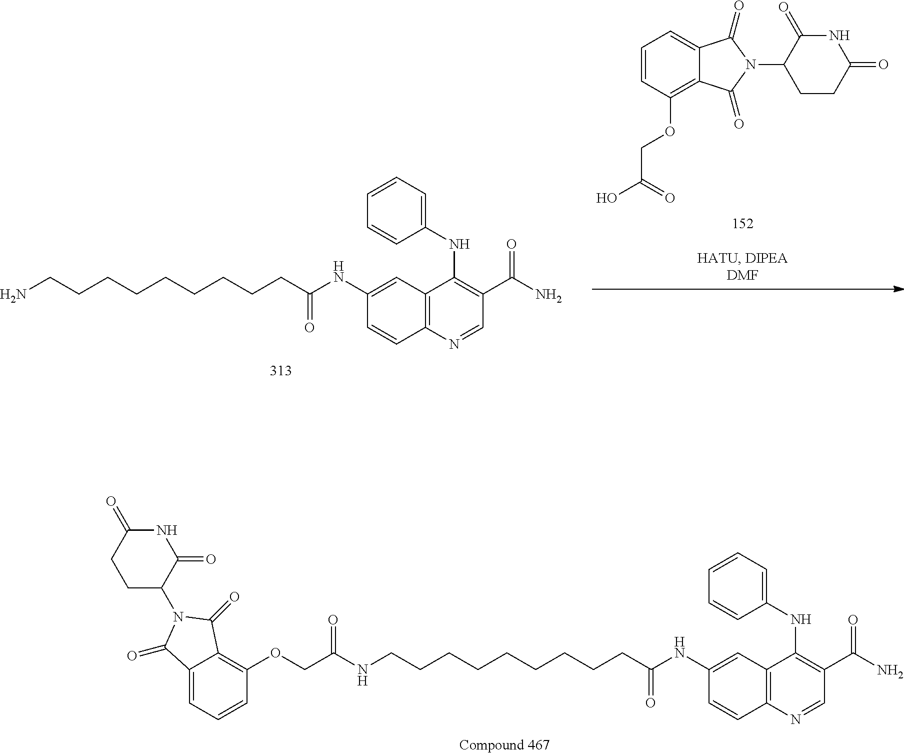 [SMILES: NCCCCCCCCCC(=O)Nc1ccc2ncc(C(N)=O)c(Nc3ccccc3)c2c1 ; O=C(O)COc1cccc2c1C(=O)N(C1CCC(=O)NC1=O)C2=O ; NC(=O)c1cnc2ccc(NC(=O)CCCCCCCCCNC(=O)COc3cccc4c3C(=O)N(C3CCC(=O)NC3=O)C4=O)cc2c1Nc1ccccc1]

Into a 25 mL single-necked round-bottomed flask containing a well-stirred solution of 6-(10-aminodecanoylamino)-4-anilino-quinoline-3-carboxamide (313, 67.35 mg, 150.48 μmol) and 2-[2-(2,6-dioxo-3-piperidyl)-1,3-dioxo-isoindolin-4-yl]oxyacetic acid (152, 50 mg, 150.48 μmol) in DMF (10 mL) were added DIPEA (58.35 mg, 451.45 μmol, 78.63 uL) and HATU (85.83 mg, 225.72 μmol) under nitrogen atmosphere at room temperature, the resulting mixture was stirred at room temperature for 16 h. After completion of reaction, 20 mL of cold water was added and stirred for 10 minutes. The resulting solid was filtered and dried to afford crude product, which was purified by reverse phase prep purification (SUNFIRE OBD C18(100×30)MM 50 Mobile phase: A: 0.1% TFA in water B: ACN) to obtain 4-anilino-6-[10-[[2-[2-(2,6-dioxo-3-piperidyl)-1,3-dioxo-isoindolin-4-yl]oxyacetyl]amino]decanoylamino]quinoline-3-carboxamide (Compound 467, 30 mg, 39.38 μmol, 26.17% yield). ¹H NMR (400 MHz, DMSO-d₆) δ 12.13 (s, 1H), 11.11 (s, 1H), 10.20 (s, 1H), 8.93 (s, 1H), 8.46 (s, 1H), 8.35 (s, 1H), 7.96-7.86 (m, 4H), 7.80 (dd, J=8.5, 7.3 Hz, 1H), 7.49 (d, J=7.3 Hz, 1H), 7.39 (dt, J=7.5, 3.5 Hz, 3H), 7.25 (t, J=7.8 Hz, 3H), 5.11 (dd, J=12.9, 5.3 Hz, 1H), 4.76 (s, 2H), 3.13 (q, J=6.7 Hz, 2H), 2.89 (ddd, J=17.3, 14.0, 5.5 Hz, 1H), 2.71-2.57 (m, 3H), 2.35-2.31 (m, 1H), 2.24 (t, J=7.3 Hz, 2H), 2.09-1.98 (m, 1H), 1.56-1.47 (m, 1H), 1.47-1.38 (m, 1H), 1.24 (s, 10H). LCMS (ES+): m/z 761 [M+H]⁺

### Synthesis of Compound 468

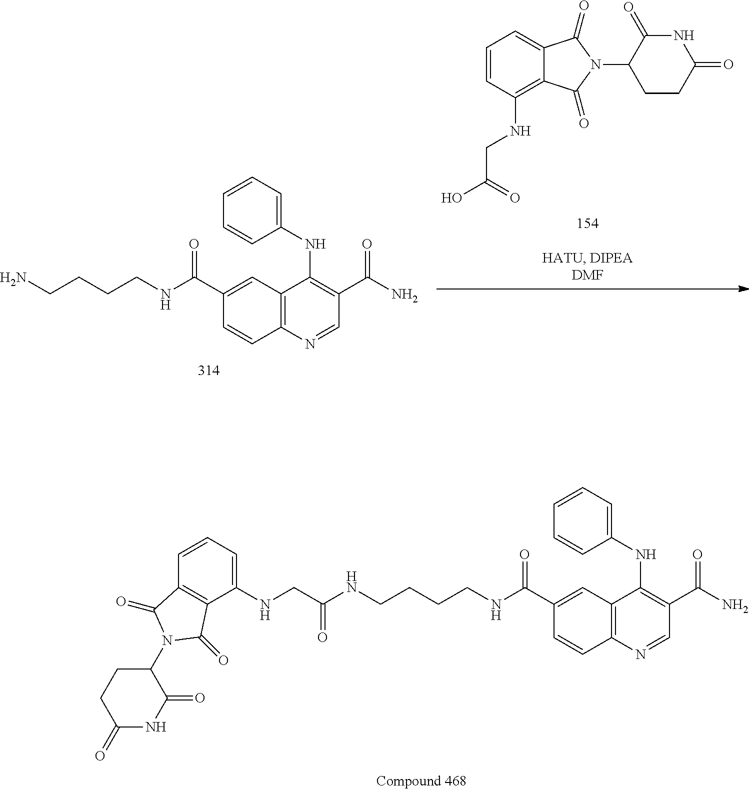 [SMILES: NCCCCNC(=O)c1ccc2ncc(C(N)=O)c(Nc3ccccc3)c2c1 ; O=C(O)CNc1cccc2c1C(=O)N(C1CCC(=O)NC1=O)C2=O ; NC(=O)c1cnc2ccc(C(=O)NCCCCNC(=O)CNc3cccc4c3C(=O)N(C3CCC(=O)NC3=O)C4=O)cc2c1Nc1ccccc1]

An oven dried round bottom flask was charged with a solution of 2-[[2-(2,6-dioxo-3-piperidyl)-1,3-dioxo-isoindolin-4-yl]amino]acetic acid (154, 30.72 mg, 92.73 μmol) in DMF (5 mL), N6-(4-aminobutyl)-4-anilino-quinoline-3,6-dicarboxamide (314, 35.00 mg, 92.73 μmol), DIPEA (59.92 mg, 463.65 μmol, 80.76 uL) and HATU (42.31 mg, 111.28 μmol) were added. The reaction mixture was stirred for 16 h at room temperature. The reaction mixture was concentrated under reduced pressure. The crude mixture was purified by reverse phase prep column chromatography (Column: HPLC(SUNFIRE OBD C18(100×30)MM 5μ) Mobile phase: A: 0.1% TFA in water B: ACN) to yield 4-anilino-N6-[4-[[2-[[2-(2,6-dioxo-3-piperidyl)-1,3-dioxo-isoindolin-4-yl]amino]acetyl]amino]butyl]quinoline-3,6-dicarboxamide (Compound 468, 22.28 mg, 30.81 μmol, 33.22% yield) as yellow solid. LCMS (ES+): m/z 691 [M+H]⁺

### Synthesis of Compound 470

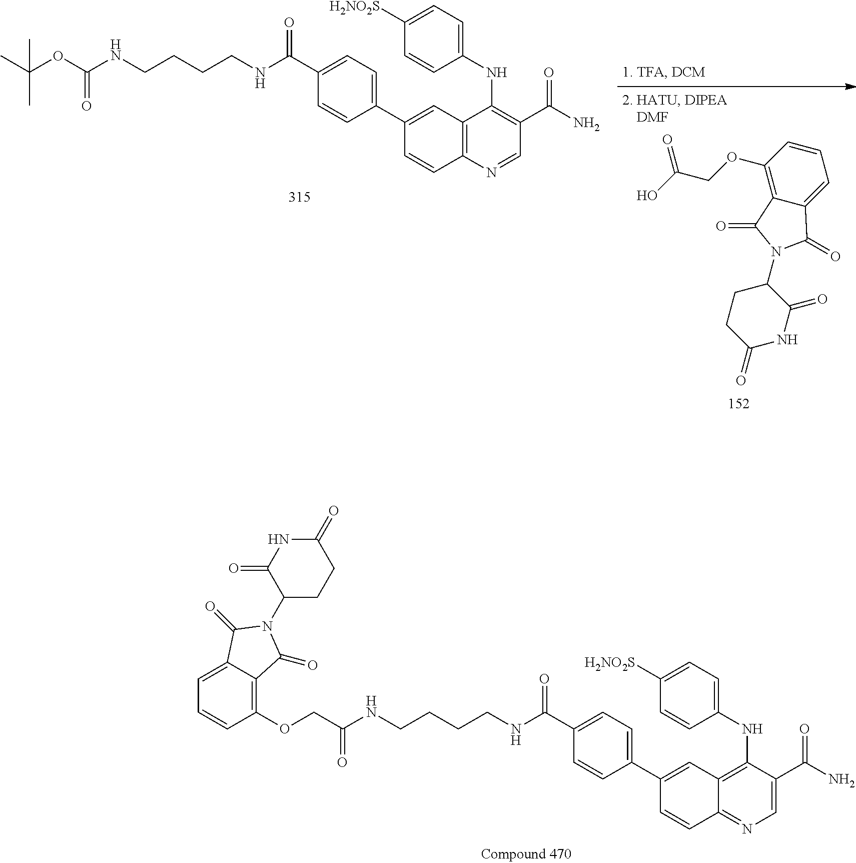 [SMILES: CC(C)(C)OC(=O)NCCCCNC(=O)c1ccc(-c2ccc3ncc(C(N)=O)c(Nc4ccc(S(N)(=O)=O)cc4)c3c2)cc1 ; O=C(O)COc1cccc2c1C(=O)N(C1CCC(=O)NC1=O)C2=O ; NC(=O)c1cnc2ccc(-c3ccc(C(=O)NCCCCNC(=O)COc4cccc5c4C(=O)N(C4CCC(=O)NC4=O)C5=O)cc3)cc2c1Nc1ccc(S(N)(=O)=O)cc1]

To a stirred solution of crude tert-butyl N-[4-[[4-[3-carbamoyl-4-(4-sulfamoylanilino)-6-quinolyl]benzoyl]amino]butyl]carbamate (315, 200 mg, 316.09 μmol) in anhydrous Dichloromethane (5 mL) was added Trifluoroacetic acid (2.96 g, 25.96 mmol, 2.0 mL) at 0° C. The reaction mixture was stirred at room temperature for 3 hours. Then reaction mixture was concentrated completely, and this crude was taken to the next step without further purification. To the stirred solution of above crude in N,N-Dimethylformamide (8 mL) was added 2-[2-(2,6-dioxo-3-piperidyl)-1,3-dioxo-isoindolin-4-yl]oxyacetic acid (152, 157.54 mg, 474.14 μmol) followed by μmol), HATU (240.37 mg, 632.18 μmol) and N,N-Diisopropylethylamine (204.26 mg, 1.58 mmol, 275.29 uL). The reaction mixture was stirred at room temperature for 16 hours. Upon completion, ice cold water (15 mL) was added and stirred for 10 minutes, and then extracted with ethyl acetate. The combined organics were dried over anhydrous sodium sulfate and then concentrated to yield the crude product, which was purified by prep-HPLC(SUNFIRE OBD C18(100×30)MM 5μ), mobile phase: A: 0.1% TFA in water B: ACN) to yield the product 6-[4-[4-[[2-[2-(2,6-dioxo-3-piperidyl)-1,3-dioxo-isoindolin-4-yl]oxyacetyl]amino]butylcarbamoyl]phenyl]-4-(4-sulfamoylanilino)quinoline-3-carboxamide (Compound 470, 4.0 mg, 4.53 μmol, 1.43% yield) as a pale yellow solid. LCMS (ES+): m/z 847 [M+H]⁺

### Example 3. HiBit Lytic Assay

### Materials

Sterile Flask: Corning® cell culture flasks surface area 150 cm², canted neck, cap (vented) (Corning, CLS430825-5EA)

### Cell Counter: Countess II Automated Cell Counter (Thermo Fisher, AMQAX1000)

### Counting Chamber Slide: Countess® Cell Counting Chamber Slides (Thermo Fisher, C10228)

### Cell Strainer: EASYSTRAINER 40 μM, FOR 50 ML TUBES (Greiner Bio-One, 542040)

Microplate: Corning® 384 Well Low Flange Black Flat Bottom Polystyrene TC-Treated Microplates, 10 per Bag, with Lid, Sterile (Corning, 3571)
Cell Dispenser: Multidrop™ Combi Reagent Dispenser (Thermo Fisher, 5840300) Standard tube dispensing cassette (Thermo Fisher, 24072670)

### Incubator: VWR® Symphony™ Air-Jacketed CO2 Incubators, Models 5.3A and 8.5A (VWR, 98000-368)

### Compound Microplate: 384-Well Low Dead Volume Microplate (Labcyte, LP-0200)

Electronic Pipette: E1-ClipTip™ Electronic Multichannel Pipettes (Thermo Fisher, various volumes)

### Plate Sealer: PlateLoc Thermal Microplate Sealer (Agilent, G5402A/G)

### Compound Dispenser: Echo® 550 Liquid Handler (Labcyte, Echo 550)

Plate Reader: Envision Multimode Plate Reader with ultra-sensitive luminescence option (Perkin Elmer, 2104-0010)

Cell Lines and Reagents

| Cell | | | | Plating | |
|---|---|---|---|---|---|
| Line | | | | Density | |
| ID | | | | (30 μL/ | Compund |
| (Scin- | Parental | | Growth | 384- | Treatment |
| amic) | Cell Line | Modification | Media | well) | Time [hr] |
| | | SMARTCAR- | RPMI | | |
| Jurkat.7 | Jurkat.1 | MTH1-wt- | 1640 +30 | 10000 | 4 |
| | | HiBiT | 10% FBS | | |

*Cell lines used are sourced from appropriate repositories (e.g. ATCC), verified free of
Mycobacterium contamination, and maintained prior to and during an experiment at 37° C., 5%
CO2, and at 95% relative humidity. Cell passage number will be limited to passage 25,

### Growth Media:

DMEM, high glucose, HEPES, no phenol red (Thermo Fisher, 21063045) with 10% Fetal Bovine Serum (Thermo Fisher, 10437036)
RPMI 1640 Medium, no phenol red (Thermo Fisher, 11835030) with 10% Fetal Bovine Serum (Thermo Fisher, 10437036)

### HiBiT Readout Reagent: Nano-Glo® HiBiT Lytic Assay System (Promega, N3050)

Trypsin: Trypsin-EDTA (0.5%), no phenol red (Thermo Fisher, 15400054)
PBS: phosphate buffered saline, 1×, pH 7.4 (Thermo Fisher, 10010049)
Trypan blue: Trypan Blue Solution, 0.4% (Thermo Fisher, 15250061)
DMSO: Dimethyl sulfoxide for molecular biology (Sigma Aldrich, D8418).

### Procedure

### Master Dose Response (MDR) Plate Preparation:

Compound is made at 10 mM, half log dilution, 11 points.
MDR plates can also be ordered from Frontier with same format.
   1. Barcode a 384LDV plate (384-well Low Dead Volume Microplate, Labcyte LP-0200) (Echo compatible) on the left side.
   2. Transfer 13.6 μL of 10 mM compound into column 1 of LDV plate (Labcyte).
   3. Fill column 2-11 and column 23 of MDR plate with 9.30 μL of DMSO.
   4. Serial dilute 3.16 fold from column 1 to 2 by transferring 4.30 μL from column 1 to column 2, and so on to column 11. *Change tip in every dilution to prevent compound carryover effect!
   5. Seal the plate with Plate Sealer, then store the MDR plate at room temperature for short term storage and in −20° C. for long term storage.

### Compound Dispensing

Cells are treated with 30 nL of each compound at 10 mM, half log dilution, 11 points with final concentration of 1000 nM or 833 nM, half log dilution, 11 points, in duplicates. Final DMSO concentration is 0.1%.
   1. Barcode the plates made on the left side and label the plates with silver marker.
   2. Dispense with the following scheme:
      a. 30 nL of compound into corresponding wells in cell plates via Echo550 with “MDR32_HiBiT16_30 nL”. Top concentration will be 1000 nM.
   3. Seal the plate with Plate Sealer.

### Cell Dispensing

Cells are dispensed at density specified in Cell Line Table in 384-well microplate.
   1. Culture cells to between 50-80% of confluence in sterile culture flasks of appropriate size.
   2. For Adherent Cell Lines:
      a. Aspirate off growth media in culture flask, and wash off the Phenol Red media with 5 mL of 1×PBS.
      b. Add 5 mL of Trypsin to the cells, incubate at 37° C. for 5 minutes.
      c. Add 5 mL of growth media to the trypsinzed cells, and resuspend.
   3. Remove 10 μL of cells, and resuspend the cells with 10 μL of Trypan Blue. Insert the solution to counting chamber slide, and count the cells via cell counter.
   4. Adjust the cell concentration to specified plating density, and strain the cells with cell strainer.
   5. Dispense the cells via E1-ClipTip™ Electronic Multichannel Pipettes or Multidrop™ Combi Reagent Dispenser with Standard tube dispensing cassette:
      a. Prime the cassette with 10-20 mL of ddH₂O.
      b. Prime the cassette with 10-20 mL of cells.
      c. Dispense the cells at low speed for 30 μL/well in Corning® 384 Well Low Flange Black Flat Bottom Polystyrene TC-Treated Microplate.
      d. Wash the cassette with 10-20 mL of ddH₂O, follow by 10-20 mL of 70% EtOH, then again with 10-20 mL of ddH₂O.
   6. Incubate the plates at 37° C.×compound treatment time.

### HiBiT Lytic Reagent Dispensing

1. If not already, bring Nano-Glo® HiBiT Lytic Buffer to room temperature.
2. Determine the amount of reagent needed. A volume of reagent equal to the volume of media in the well should be added (i.e. 30 μL of cells will need 30 μL of HiBiT Lytic reagent).
3. Into a given volume of buffer, dilute the substrate 50-fold and the LgBiT protein 100-fold. For example, to make 10 ml of reagent, add the following:
   10 ml Nano-Glo® HiBiT Lytic Buffer
   200 μl Nano-Glo® HiBiT Lytic Substrate
   100 μl LgBiT Protein
The reagent should be reconstituted immediately before use. However, once reconstituted, it should take about 4 hours for the activity to decrease 10%.
4. Cool down the cell plate to room temperature for 10-15 minutes without lid.
5. Dispense the HiBiT Lytic Reagent via E1-ClipTip™ Electronic Multichannel Pipettes or Multidrop™ Combi Reagent Dispenser with Standard tube dispensing cassette:
   a. Prime the cassette with 10-20 mL of ddH₂O.
   b. Prime the cassette with 10-20 mL of CTG 2.0.
   c. Dispense the HiBiT Lytic Reagent at medium speed for 30 μL/well in cell plate to column 1-23 (column 24 will be used as plate background).
   d. Wash the cassette with 10-20 mL of ddH₂O, follow by 10-20 mL of 70% EtOH, then again with 10-20 mL of ddH₂O.
6. Incubate the cell plate at room temperature for 10 minutes without lid.
7. Read the NanoLuc signal with Envision Multimode Plate Reader with ultra-sensitive luminescence method.

### Data Analysis & QC

Data Normalization
   Negative control value (N)
      N is computed per cell line from positive (P) media-only controls and DMSO treated cells read at the assay time-point:
      N=Average (DMSO)−Average (P)
   Response percent (%)
      Response % of compound-treated samples are calculated by normalizing the signal with P and DMSO treated negative (N) controls on the same microtiter plate:
      R %=100×(Signal−Average (P))/N
The response % is thus 100% if the NanoLuc signal equals that of the DMSO treated controls, 0% if it equals that of the media-only controls.
Curve fit—4 parametric logistic fit
The data was fitted by using the in-house data analysis software (Scinamic). The fit was performed through minimization of the root mean squared error between observed and calculated values of the four parameter logistic equation using the simplex optimizer of the Apache Commons Math library. Boundary conditions for the fit parameters were set as: top (T) was constrained to be between 80% and 120% response, bottom (B) to be between −100% and 80% response, Hill slope (H) between −3 and −0.3, inflection point (IP) unrestricted. These default parameters could be changed by the user for individual curves if appropriate.

IC50
IC50s were computed as the concentrations where the fitted curves cross the 50% response level.
If a calculated value was below the lowest tested concentration Cₘᵢₙ the value was set to ‘<Cₘᵢₙ’. If a calculated value was above the highest tested concentration Cₘₐₓ or no value exists because the bottom of the curve is above the given response level, the value was set to ‘>Cₘₐₓ’. Averages and standard deviations were computed from replicates of the experiment.

### Results

Using the above assay the HiBit DC50 data was determined for the compounds in the Tables below.

### Example 4. HTFC Assay

### Materials

Barcoded 96-well V-bottom polypropylene plates (Greiner Bio-One #651261)

### Sterile lid (Greiner Bio-One #656171)

LDV MDR plate prepared from Frontier

### Fixation/Permeabilization Kit (BD #554714)

FITC anti-HA.11 epitope tag antibody (Biolegend #901507)
FITC Mouse IgG1 k isotope control antibody (Biolegend #400108)

### Echo Dispensing

1. Load Echo dose response pick list into appropriate file.

Dry dispense 10 nL/well of compound+90 μL/well of DMSO into 96-well V-bottom plates: 4 compounds/plate in 11-point dose response in biological duplicates. Column 12 as DMSO controls.

### Cell plating

1. Plate Jurkat.2 (SMART-CAR_MTH1-WT) cells with Combi on top of dry dispensed compound: 500,000 cells/mL, 100 uL/well
2. Incubate for 4 hours at 37° C.
3. Fixation, permeabilization, and staining:
   a. Centrifuge at 1500 rpm for 7 minutes, remove supernatant.
   b. Add 100 μl of Fixation/Permeabilization Solution, seal plates, vortex, pulse spin, and incubate at 4° C. for 30 minutes.
   c. Centrifuge at 1500 rpm for 7 minutes, remove supernatant.
   d. Dilute BD Perm/Wash Buffer to 1× with milliQ water.
   e. Wash with 200 uL of BD Perm/Wash Buffer, seal plates, vortex.
   f. Centrifuge at 1500 rpm for 7 minutes, remove supernatant.
   g. Wash with 200 uL of BD Perm/Wash Buffer, seal plates, vortex. (Option to stop, store at 4° C. if needed)
   h. Centrifuge at 1500 rpm for 7 minutes, remove supernatant.
   i. Add 50 uL BD Perm/Wash Buffer containing 1:1000 of HA-FITC (Well A12:D12) or IgG-FITC (Well F12:H12) directly conjugated antibody, seal plates, vortex, pulse spin, and incubate at 4° C. for 1 hour.
   j. Centrifuge at 1500 rpm for 7 minutes, remove supernatant.
   k. Wash with 200 uL of BD Perm/Wash Buffer, seal plates, vortex.
4. Centrifuge at 1500 rpm for 7 minutes, remove supernatant.
   a. Resuspend in 200 ul of BD Perm/Wash Buffer. (Option to stop, store at 4° C. if needed)

### Guava Acquisition and Scinamic Analysis

1. Use analysis from template HTFC file.
2. Export statistics by going to Tools>Group stats, save as a .csv file with the plate barcode as the name.
3. Remove all columns from this file except for “Well”, “Sample ID”, “Date”, and “HA+.Mean.Y Mean for HA+ gated by P01.cells”.
4. Create a New DB Analysis in Scinamic using the “HTFC-Cell Pharm” template.
5. Load Incyte file “barcode”.csv.
6. Load Echo file with the generated Echo report (P:\LabData\Echo\Project\SMARTCAR\Echo Report\Labcyte Echo Dose-Response)
7. Edit Platemap.xlsx with the plate barcodes.
8. Load Plate Map with this file.
9. Cleanup compound Ids, mask control outliers, and assign curves.

### Results

Using the above assay the HTFC DC50 data was determined for the compounds in Table 2 below.

### Example 5: HiBit DC50 Data for Representative Compounds of the Present Invention

TABLE 1

| | | HiBit |
|---|---|---|
| Cmpd | | DC50 |
| # | Structure & Name | (nM) |
| 200 | 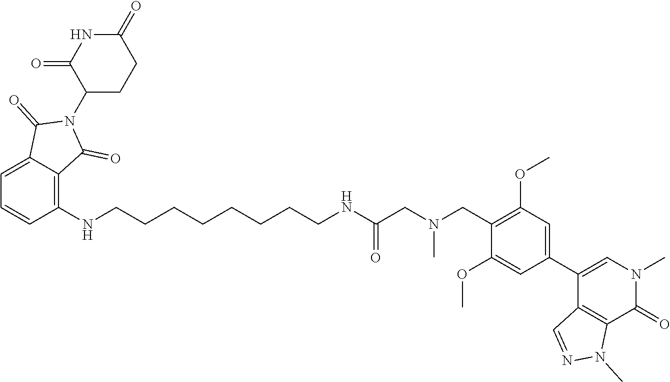 [SMILES: COc1cc(-c2cn(C)c(=O)c3c2cnn3C)cc(OC)c1CN(C)CC(=O)NCCCCCCCCNc1cccc2c1C(=O)N(C1CCC(=O)NC1=O)C2=O] | +++ |
| | 2-((4-(1,6-dimethyl-7-oxo-6,7-dihydro-1H-pyrazolo[3,4-c]pyridin-4-yl)-2,6- | |
| | dimethoxybenzyl)(methyl)amino)-N-(8-((2-(2,6-dioxopiperidin-3-yl)-1,3- | |
| | dioxoisoindolin-4-yl)amino)octyl)acetamide | |
| 201 | 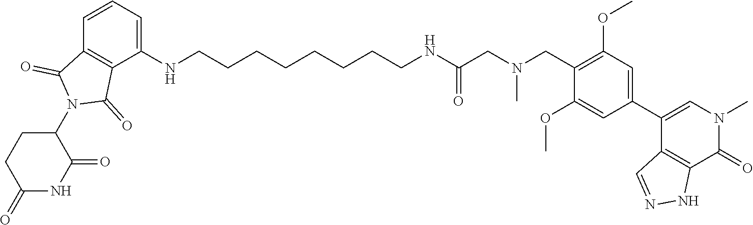 [SMILES: COc1cc(-c2cn(C)c(=O)c3[nH]ncc23)cc(OC)c1CN(C)CC(=O)NCCCCCCCCNc1cccc2c1C(=O)N(C1CCC(=O)NC1=O)C2=O] | +++ |
| | 2-((2,6-dimethoxy-4-(6-methyl-7-oxo-6,7-dihydro-1H-pyrazolo[3,4-c]pyridin- | |
| | 4-yl)benzyl)(methyl)amino)-N-(8-((2-(2,6-dioxopiperidin-3-yl)-1,3- | |
| | dioxoisoindolin-4-yl)amino)octyl)acetamide | |
| 202 | 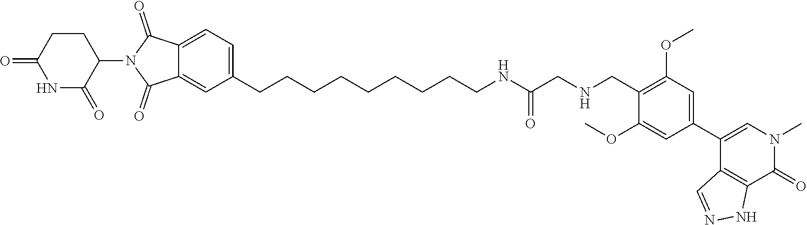 [SMILES: COc1cc(-c2cn(C)c(=O)c3[nH]ncc23)cc(OC)c1CNCC(=O)NCCCCCCCCCc1ccc2c(c1)C(=O)N(C1CCC(=O)NC1=O)C2=O] | +++ |
| | 2-((2,6-dimethoxy-4-(6-methyl-7-oxo-6,7-dihydro-1H-pyrazolo[3,4-c]pyridin- | |
| | 4-yl)benzyl)amino)-N-(9-(2-(2,6-dioxopiperidin-3-yl)-1,3-dioxoisoindolin-5- | |
| | yl)nonyl)acetamide | |
| 203 | 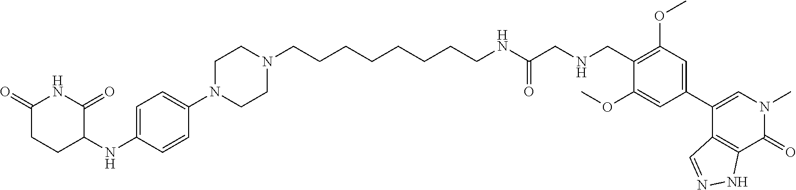 [SMILES: COc1cc(-c2cn(C)c(=O)c3[nH]ncc23)cc(OC)c1CNCC(=O)NCCCCCCCCN1CCN(c2ccc(NC3CCC(=O)NC3=O)cc2)CC1] | +++ |
| | 2-((2,6-dimethoxy-4-(6-methyl-7-oxo-6,7-dihydro-1H-pyrazolo[3,4-c]pyridin- | |
| | 4-yl)benzyl)amino)-N-(8-(4-(4-((2,6-dioxopiperidin-3- | |
| | yl)amino)phenyl)piperazin-1-yl)octyl)acetamide | |
| 204 | 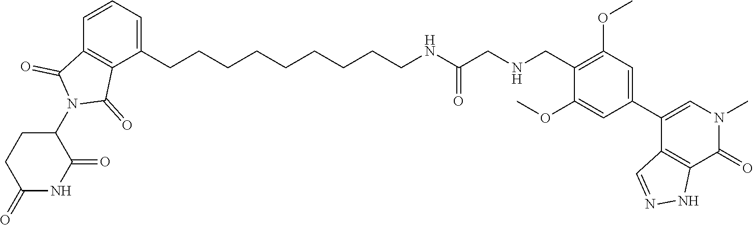 [SMILES: COc1cc(-c2cn(C)c(=O)c3[nH]ncc23)cc(OC)c1CNCC(=O)NCCCCCCCCCc1cccc2c1C(=O)N(C1CCC(=O)NC1=O)C2=O] | +++ |
| | 2-((2,6-dimethoxy-4-(6-methyl-7-oxo-6,7-dihydro-1H-pyrazolo[3,4-c]pyridin- | |
| | 4-yl)benzyl)amino)-N-(9-(2-(2,6-dioxopiperidin-3-yl)-1,3-dioxoisoindolin-4- | |
| | yl)nonyl)acetamide | |
| 206 | 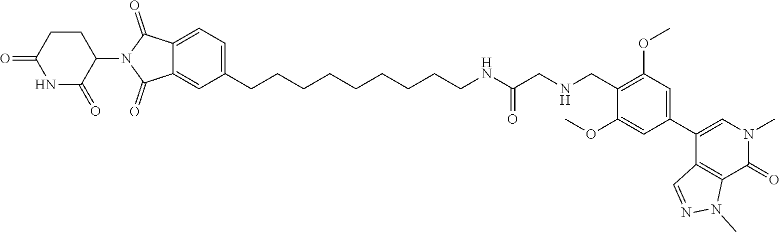 [SMILES: COc1cc(-c2cn(C)c(=O)c3c2cnn3C)cc(OC)c1CNCC(=O)NCCCCCCCCCc1ccc2c(c1)C(=O)N(C1CCC(=O)NC1=O)C2=O] | +++ |
| | 2-((4-(1,6-dimethyl-7-oxo-6,7-dihydro-1H-pyrazolo[3,4-c]pyridin-4-yl)-2,6- | |
| | dimethoxybenzyl)amino)-N-(9-(2-(2,6-dioxopiperidin-3-yl)-1,3- | |
| | dioxoisoindolin-5-yl)nonyl)acetamide | |
| 207 | 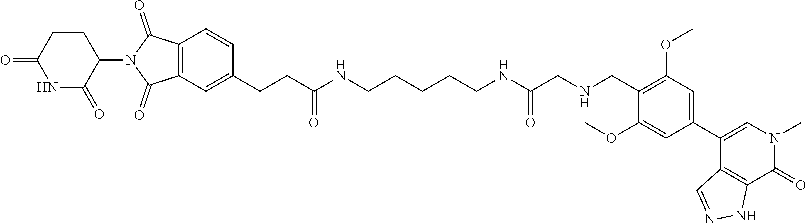 [SMILES: COc1cc(-c2cn(C)c(=O)c3[nH]ncc23)cc(OC)c1CNCC(=O)NCCCCCNC(=O)CCc1ccc2c(c1)C(=O)N(C1CCC(=O)NC1=O)C2=O] | +++ |
| | N-(5-(2-((2,6-dimethoxy-4-(6-methyl-7-oxo-6,7-dihydro-1H-pyrazolo[3,4- | |
| | c]pyridin-4-yl)benzyl)amino)acetamido)pentyl)-3-(2-(2,6-dioxopiperidin-3-yl)- | |
| | 1,3-dioxoisoindolin-5-yl)propanamide | |
| 208 | 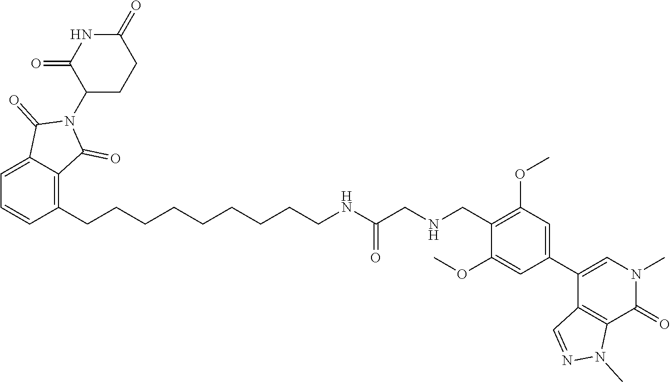 [SMILES: COc1cc(-c2cn(C)c(=O)c3c2cnn3C)cc(OC)c1CNCC(=O)NCCCCCCCCCc1cccc2c1C(=O)N(C1CCC(=O)NC1=O)C2=O] | +++ |
| | 2-((4-(1,6-dimethyl-7-oxo-6,7-dihydro-1H-pyrazolo[3,4-c]pyridin-4-yl)-2,6- | |
| | dimethoxybenzyl)amino)-N-(9-(2-(2,6-dioxopiperidin-3-yl)-1,3- | |
| | dioxoisoindolin-4-yl)nonyl)acetamide | |
| 209 | 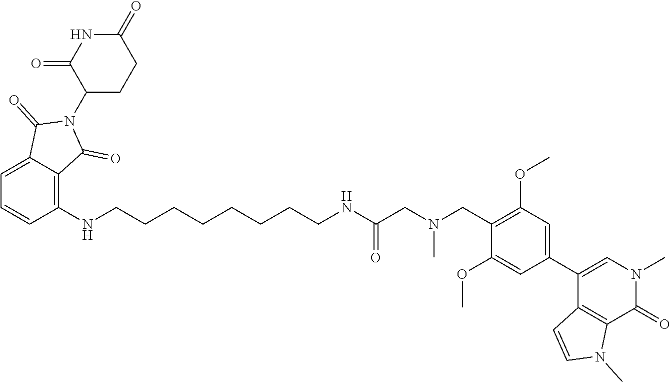 [SMILES: COc1cc(-c2cn(C)c(=O)c3c2ccn3C)cc(OC)c1CN(C)CC(=O)NCCCCCCCCNc1cccc2c1C(=O)N(C1CCC(=O)NC1=O)C2=O] | +++ |
| | 2-((4-(1,6-dimethyl-7-oxo-6,7-dihydro-1H-pyrrolo[2,3-c]pyridin-4-yl)-2,6- | |
| | dimethoxybenzyl)(methyl)amino)-N-(8-((2-(2,6-dioxopiperidin-3-yl)-1,3- | |
| | dioxoisoindolin-4-yl)amino)octyl)acetamide | |
| 218 | 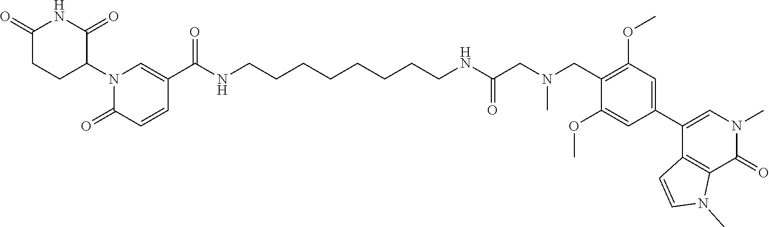 [SMILES: COc1cc(-c2cn(C)c(=O)c3c2ccn3C)cc(OC)c1CN(C)CC(=O)NCCCCCCCCNC(=O)c1ccc(=O)n(C2CCC(=O)NC2=O)c1] | ++ |
| | N-(8-(2-((4-(1,6-dimethyl-7-oxo-6,7-dihydro-1H-pyrrolo[2,3-c]pyridin-4-yl)- | |
| | 2,6-dimethoxybenzyl)(methyl)amino)acetamido)octyl)-1-(2,6-dioxopiperidin-3- | |
| | yl)-6-oxo-1,6-dihydropyridine-3-carboxamide | |
| 220 | 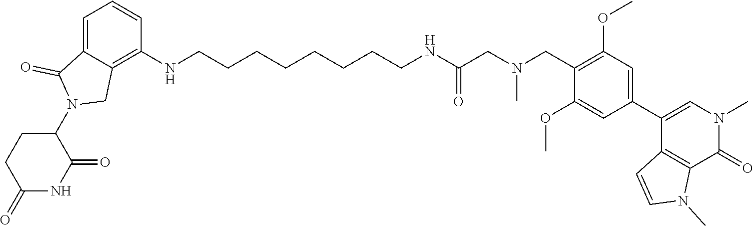 [SMILES: COc1cc(-c2cn(C)c(=O)c3c2ccn3C)cc(OC)c1CN(C)CC(=O)NCCCCCCCCNc1cccc2c1CN(C1CCC(=O)NC1=O)C2=O] | +++ |
| | 2-((4-(1,6-dimethyl-7-oxo-6,7-dihydro-1H-pyrrolo[2,3-c]pyridin-4-yl)-2,6- | |
| | dimethoxybenzyl)(methyl)amino)-N-(8-((2-(2,6-dioxopiperidin-3-yl)-1- | |
| | oxoisoindolin-4-yl)amino)octyl)acetamide | |
| 221 | 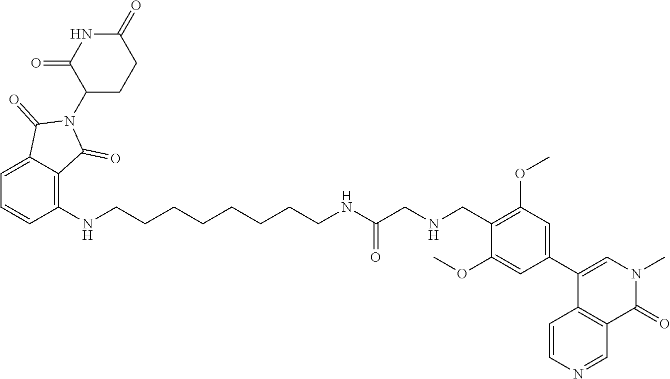 [SMILES: COc1cc(-c2cn(C)c(=O)c3cnccc23)cc(OC)c1CNCC(=O)NCCCCCCCCNc1cccc2c1C(=O)N(C1CCC(=O)NC1=O)C2=O] | +++ |
| | 2-((2,6-dimethoxy-4-(2-methyl-1-oxo-1,2-dihydro-2,7-naphthyridin-4- | |
| | yl)benzyl)amino)-N-(8-((2-(2,6-dioxopiperidin-3-yl)-1,3-dioxoisoindolin-4- | |
| | yl)amino)octyl)acetamide | |
| 222 | 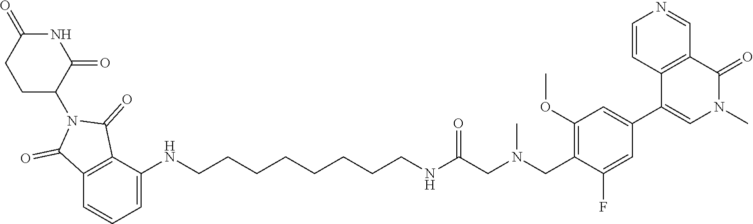 [SMILES: COc1cc(-c2cn(C)c(=O)c3cnccc23)cc(F)c1CN(C)CC(=O)NCCCCCCCCNc1cccc2c1C(=O)N(C1CCC(=O)NC1=O)C2=O] | +++ |
| | N-(8-((2-(2,6-dioxopiperidin-3-yl)-1,3-dioxoisoindolin-4-yl)amino)octyl)-2-((2- | |
| | fluoro-6-methoxy-4-(2-methyl-1-oxo-1,2-dihydro-2,7-naphthyridin-4- | |
| | yl)benzyl)(methyl)amino)acetamide | |
| 223 | 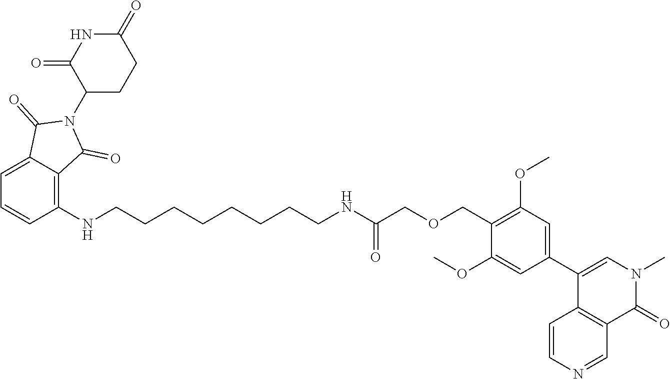 [SMILES: COc1cc(-c2cn(C)c(=O)c3cnccc23)cc(OC)c1COCC(=O)NCCCCCCCCNc1cccc2c1C(=O)N(C1CCC(=O)NC1=O)C2=O] | +++ |
| | 2-((2,6-dimethoxy-4-(2-methyl-1-oxo-1,2-dihydro-2,7-naphthyridin-4- | |
| | yl)benzyl)oxy)-N-(8-((2-(2,6-dioxopiperidin-3-yl)-1,3-dioxoisoindolin-4- | |
| | yl)amino)octyl)acetamide | |
| 224 | 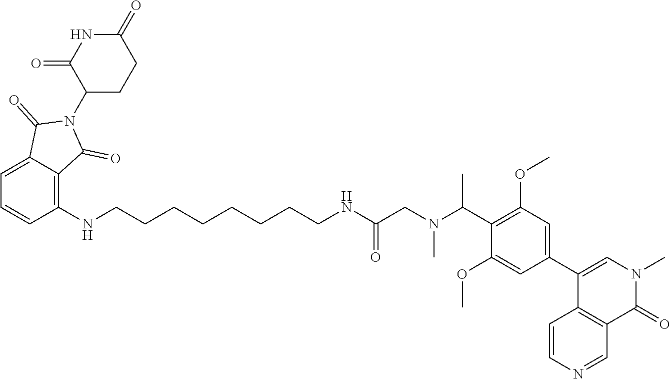 [SMILES: COc1cc(-c2cn(C)c(=O)c3cnccc23)cc(OC)c1C(C)N(C)CC(=O)NCCCCCCCCNc1cccc2c1C(=O)N(C1CCC(=O)NC1=O)C2=O] | +++ |
| | 2-((1-(2,6-dimethoxy-4-(2-methyl-1-oxo-1,2-dihydro-2,7-naphthyridin-4- | |
| | yl)phenyl)ethyl)(methyl)amino)-N-(8-((2-(2,6-dioxopiperidin-3-yl)-1,3- | |
| | dioxoisoindolin-4-yl)amino)octyl)acetamide | |
| 225 | 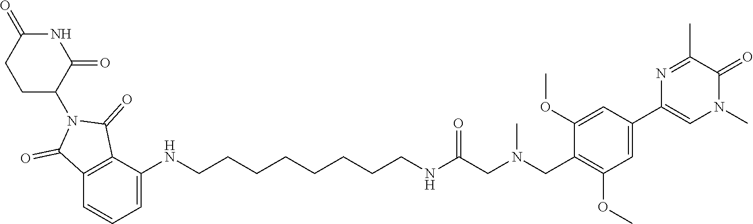 [SMILES: COc1cc(-c2cn(C)c(=O)c(C)n2)cc(OC)c1CN(C)CC(=O)NCCCCCCCCNc1cccc2c1C(=O)N(C1CCC(=O)NC1=O)C2=O] | +++ |
| | 2-((4-(4,6-dimethyl-5-oxo-4,5-dihydropyrazin-2-yl)-2,6- | |
| | dimethoxybenzyl)(methyl)amino)-N-(8-((2-(2,6-dioxopiperidin-3-yl)-1,3- | |
| | dioxoisoindolin-4-yl)amino)octyl)acetamide | |

In the above table +++ is <100 nM and ++ is <1000 nM

### Example 6: HiBit and HTFC DC50 Data for Representative Compounds of the Present Invention

TABLE 2

| Cmpd | | DC50 | DC50 |
|---|---|---|---|
| # | Structure | HiBit | HTFC |
| 300 | 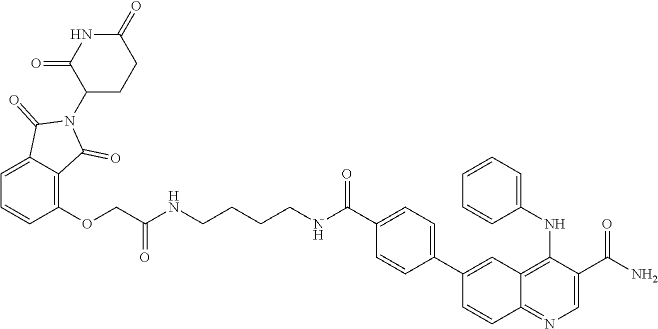 [SMILES: NC(=O)c1cnc2ccc(-c3ccc(C(=O)NCCCCNC(=O)COc4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)oxy)acetamido)butyl)carbamoyl)phenyl)-4-(phenylamino)quinoline-3- | | |
| | carboxamide | | |
| 301 | 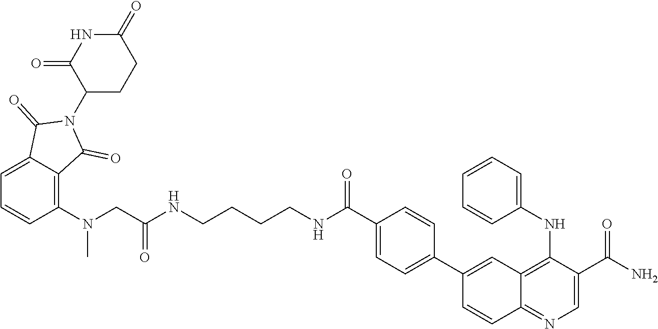 [SMILES: CN(CC(=O)NCCCCNC(=O)c1ccc(-c2ccc3ncc(C(N)=O)c(Nc4ccccc4)c3c2)cc1)c1cccc2c1C(=O)N(C1CCC(=O)NC1=O)C2=O] | +++ | |
| | 6-(4-((4-(2-((2-(2,6-dioxopiperidin-3-yl)-1,3-dioxoisoindolin-4- | | |
| | yl)(methyl)amino)acetamido)butyl)carbamoyl)phenyl)-4- | | |
| | (phenylamino)quinoline-3-carboxamide | | |
| 302 | 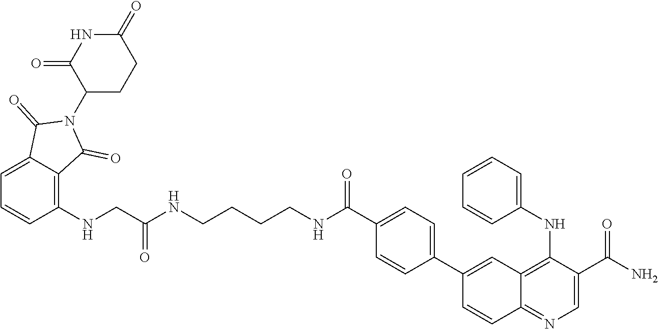 [SMILES: NC(=O)c1cnc2ccc(-c3ccc(C(=O)NCCCCNC(=O)CNc4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)amino)acetamido)butyl)carbamoyl)phenyl)-4- | | |
| | (phenylamino)quinoline-3-carboxamide | | |
| 303 | 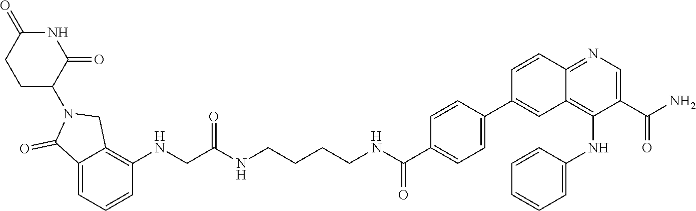 [SMILES: NC(=O)c1cnc2ccc(-c3ccc(C(=O)NCCCCNC(=O)CNc4cccc5c4CN(C4CCC(=O)NC4=O)C5=O)cc3)cc2c1Nc1ccccc1] | +++ | |
| | 6-(4-((4-(2-((2-(2,6-dioxopiperidin-3-yl)-1-oxoisoindolin-4- | | |
| | yl)amino)acetamido)butyl)carbamoyl)phenyl)-4- | | |
| | (phenylamino)quinoline-3-carboxamide | | |
| 306 | 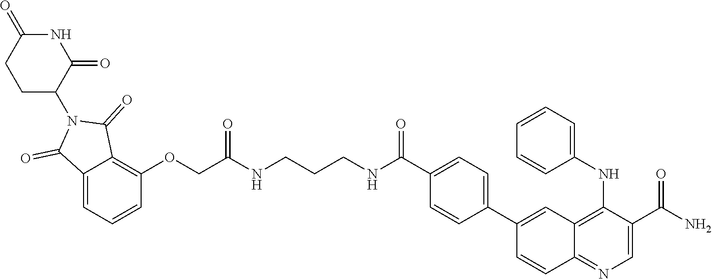 [SMILES: NC(=O)c1cnc2ccc(-c3ccc(C(=O)NCCCNC(=O)COc4cccc5c4C(=O)N(C4CCC(=O)NC4=O)C5=O)cc3)cc2c1Nc1ccccc1] | +++ | |
| | 6-(4-((3-(2-((2-(2,6-dioxopiperidin-3-yl)-1,3-dioxoisoindolin-4- | | |
| | yl)oxy)acetamido)propyl)carbamoyl)phenyl)-4-(phenylamino)quinoline- | | |
| | 3-carboxamide | | |
| 307 | 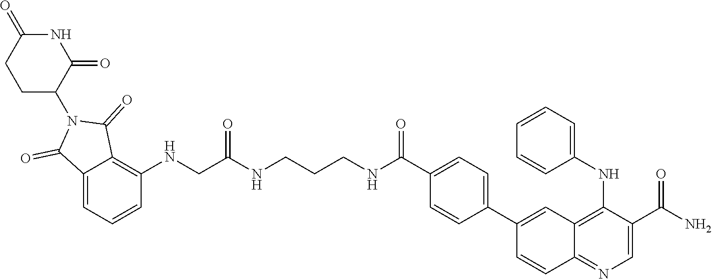 [SMILES: NC(=O)c1cnc2ccc(-c3ccc(C(=O)NCCCNC(=O)CNc4cccc5c4C(=O)N(C4CCC(=O)NC4=O)C5=O)cc3)cc2c1Nc1ccccc1] | +++ | |
| | 6-(4-((3-(2-((2-(2,6-dioxopiperidin-3-yl)-1,3-dioxoisoindolin-4- | | |
| | yl)amino)acetamido)propyl)carbamoyl)phenyl)-4- | | |
| | (phenylamino)quinoline-3-carboxamide | | |
| 308 | 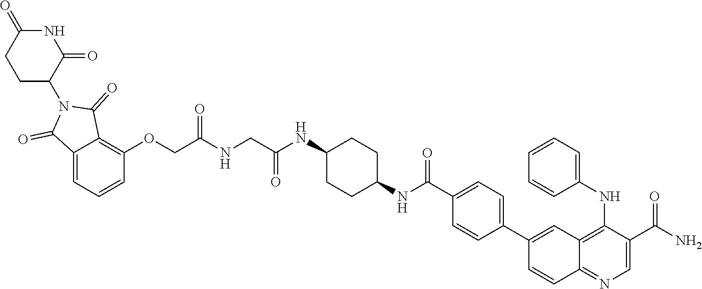 [SMILES: NC(=O)c1cnc2ccc(-c3ccc(C(=O)N[C@H]4CC[C@@H](NC(=O)CNC(=O)COc5cccc6c5C(=O)N(C5CCC(=O)NC5=O)C6=O)CC4)cc3)cc2c1Nc1ccccc1] | +++ | |
| | 6-(4-(((1s,4s)-4-(2-(2-((2-(2,6-dioxopiperidin-3-yl)-1,3-dioxoisoindolin- | | |
| | 4-yl)oxy)acetamido)acetamido)cyclohexyl)carbamoyl)phenyl)-4- | | |
| | (phenylamino)quinoline-3-carboxamide | | |
| 309 | 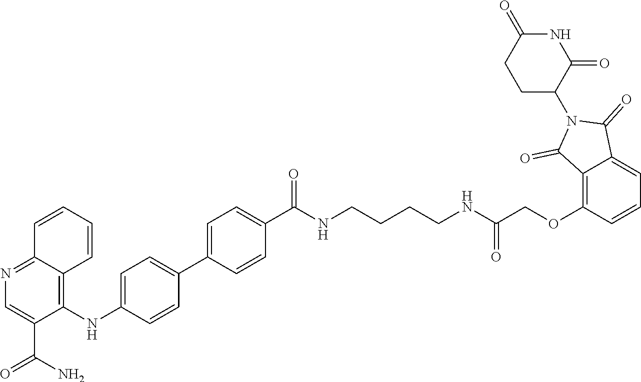 [SMILES: NC(=O)c1cnc2ccccc2c1Nc1ccc(-c2ccc(C(=O)NCCCCNC(=O)COc3cccc4c3C(=O)N(C3CCC(=O)NC3=O)C4=O)cc2)cc1] | +++ | |
| | 4-((4′-((4-(2-((2-(2,6-dioxopiperidin-3-yl)-1,3-dioxoisoindolin-4- | | |
| | yl)oxy)acetamido)butyl)carbamoyl)-[1,1′-biphenyl]-4- | | |
| | yl)amino)quinoline-3-carboxamide | | |
| 310 | 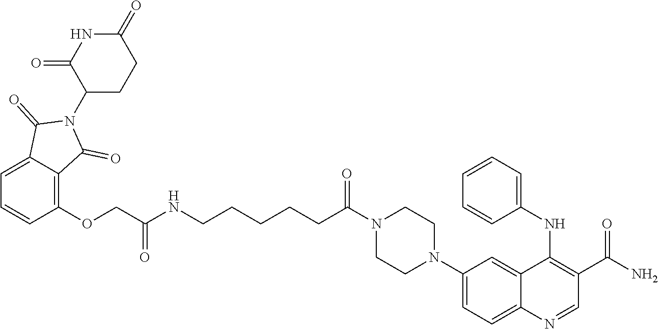 [SMILES: NC(=O)c1cnc2ccc(N3CCN(C(=O)CCCCCNC(=O)COc4cccc5c4C(=O)N(C4CCC(=O)NC4=O)C5=O)CC3)cc2c1Nc1ccccc1] | ++ | |
| | 6-(4-(6-(2-((2-(2,6-dioxopiperidin-3-yl)-1,3-dioxoisoindolin-4- | | |
| | yl)oxy)acetamido)hexanoyl)piperazin-1-yl)-4-(phenylamino)quinoline-3- | | |
| | carboxamide | | |
| 311 | 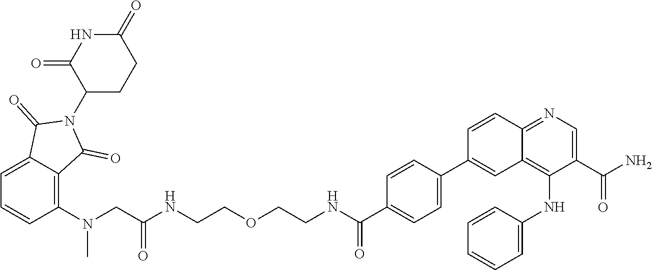 [SMILES: CN(CC(=O)NCCOCCNC(=O)c1ccc(-c2ccc3ncc(C(N)=O)c(Nc4ccccc4)c3c2)cc1)c1cccc2c1C(=O)N(C1CCC(=O)NC1=O)C2=O] | ++ | |
| | 6-(4-((2-(2-(2-((2-(2,6-dioxopiperidin-3-yl)-1,3-dioxoisoindolin-4- | | |
| | yl)(methyl)amino)acetamido)ethoxy)ethyl)carbamoyl)phenyl)-4- | | |
| | (phenylamino)quinoline-3-carboxamide | | |
| 312 | 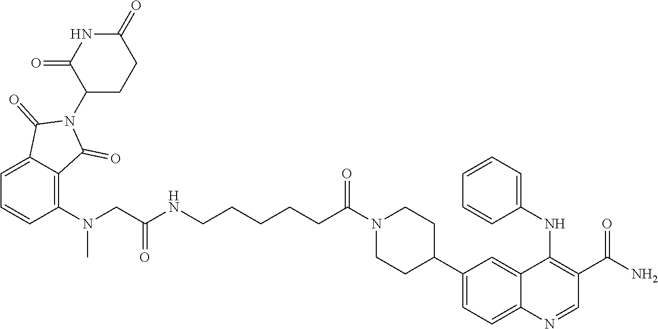 [SMILES: CN(CC(=O)NCCCCCC(=O)N1CCC(c2ccc3ncc(C(N)=O)c(Nc4ccccc4)c3c2)CC1)c1cccc2c1C(=O)N(C1CCC(=O)NC1=O)C2=O] | ++ | |
| | 6-(1-(6-(2-((2-(2,6-dioxopiperidin-3-yl)-1,3-dioxoisoindolin-4- | | |
| | yl)(methyl)amino)acetamido)hexanoyl)piperidin-4-yl)-4- | | |
| | (phenylamino)quinoline-3-carboxamide | | |
| 340 | 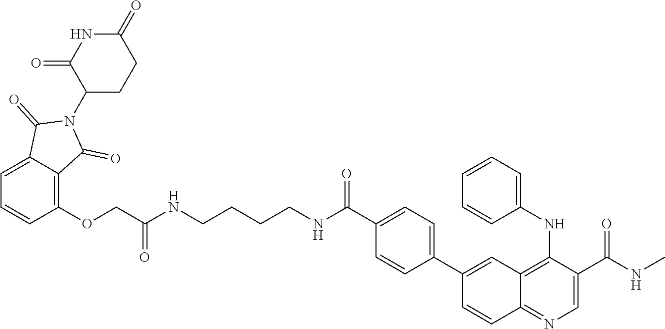 [SMILES: CNC(=O)c1cnc2ccc(-c3ccc(C(=O)NCCCCNC(=O)COc4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)oxy)acetamido)butyl)carbamoyl)phenyl)-N-methyl-4- | | |
| | (phenylamino)quinoline-3-carboxamide | | |
| 341 | 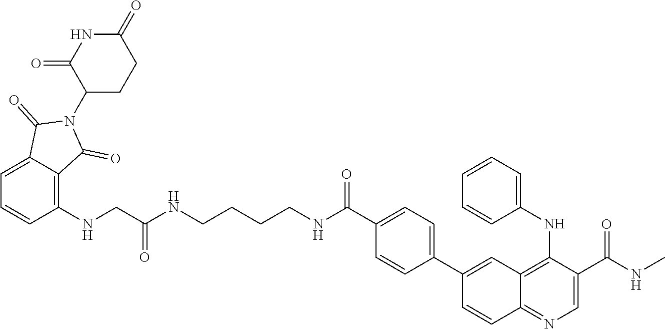 [SMILES: CNC(=O)c1cnc2ccc(-c3ccc(C(=O)NCCCCNC(=O)CNc4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)amino)acetamido)butyl)carbamoyl)phenyl)-N-methyl-4- | | |
| | (phenylamino)quinoline-3-carboxamide | | |
| 343 | 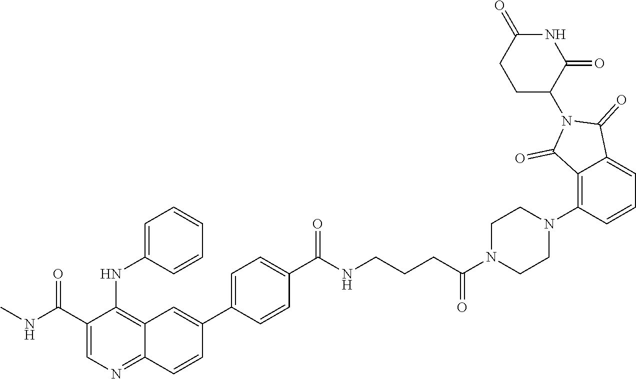 [SMILES: CNC(=O)c1cnc2ccc(-c3ccc(C(=O)NCCCC(=O)N4CCN(c5cccc6c5C(=O)N(C5CCC(=O)NC5=O)C6=O)CC4)cc3)cc2c1Nc1ccccc1] | +++ | |
| | 6-(4-((4-(4-(2-(2,6-dioxopiperidin-3-yl)-1,3-dioxoisoindolin-4- | | |
| | yl)piperazin-1-yl)-4-oxobutyl)carbamoyl)phenyl)-N-methyl-4- | | |
| | (phenylamino)quinoline-3-carboxamide | | |
| 344 | 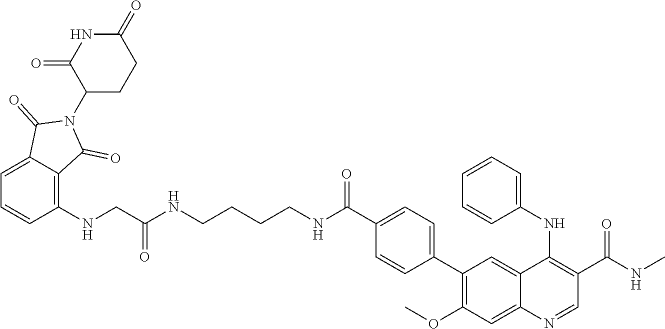 [SMILES: CNC(=O)c1cnc2cc(OC)c(-c3ccc(C(=O)NCCCCNC(=O)CNc4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)amino)acetamido)butyl)carbamoyl)phenyl)-7-methoxy-N-methyl-4- | | |
| | (phenylamino)quinoline-3-carboxamide | | |
| 345 | 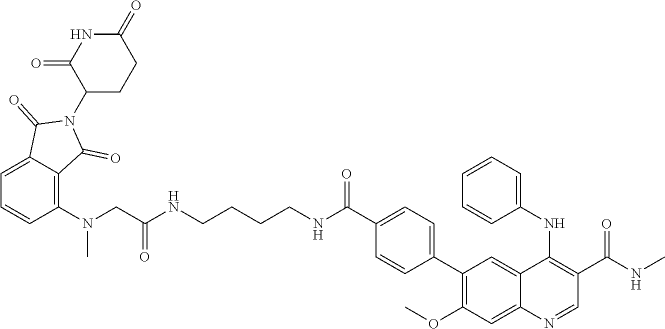 [SMILES: CNC(=O)c1cnc2cc(OC)c(-c3ccc(C(=O)NCCCCNC(=O)CN(C)c4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)(methyl)amino)acetamido)butyl)carbamoyl)phenyl)-7-methoxy-N- | | |
| | methyl-4-(phenylamino)quinoline-3-carboxamide | | |
| 346 | 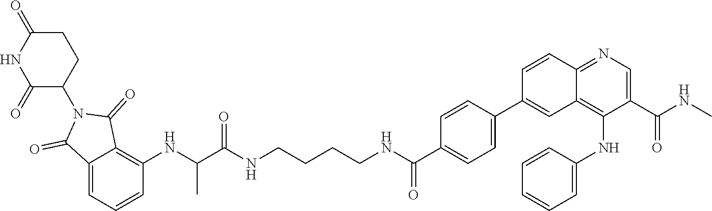 [SMILES: CNC(=O)c1cnc2ccc(-c3ccc(C(=O)NCCCCNC(=O)C(C)Nc4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)amino)propanamido)butyl)carbamoyl)phenyl)-N-methyl-4- | | |
| | (phenylamino)quinoline-3-carboxamide | | |
| 347 | 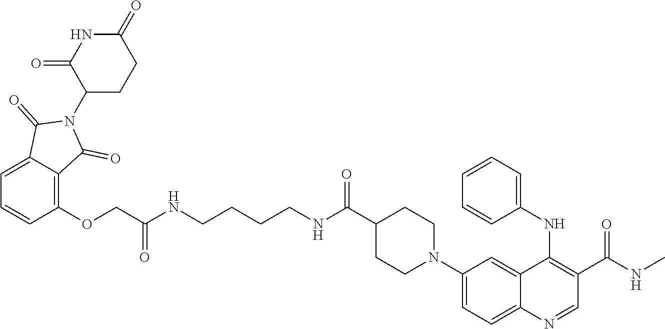 [SMILES: CNC(=O)c1cnc2ccc(N3CCC(C(=O)NCCCCNC(=O)COc4cccc5c4C(=O)N(C4CCC(=O)NC4=O)C5=O)CC3)cc2c1Nc1ccccc1] | +++ | |
| | 6-(4-((4-(2-((2-(2,6-dioxopiperidin-3-yl)-1,3-dioxoisoindolin-4- | | |
| | yl)oxy)acetamido)butyl)carbamoyl)piperidin-1-yl)-N-methyl-4- | | |
| | (phenylamino)quinoline-3-carboxamide | | |
| 348 | 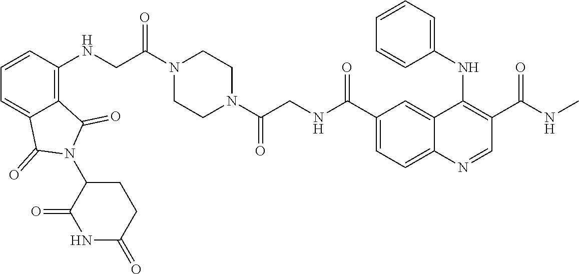 [SMILES: CNC(=O)c1cnc2ccc(C(=O)NCC(=O)N3CCN(C(=O)CNc4cccc5c4C(=O)N(C4CCC(=O)NC4=O)C5=O)CC3)cc2c1Nc1ccccc1] | | +++ |
| | N6-(2-(4-((2-(2,6-dioxopiperidin-3-yl)-1,3-dioxoisoindolin-4- | | |
| | yl)glycyl)piperazin-1-yl)-2-oxoethyl)-N3-methyl-4- | | |
| | (phenylamino)quinoline-3,6-dicarboxamide | | |
| 350 | 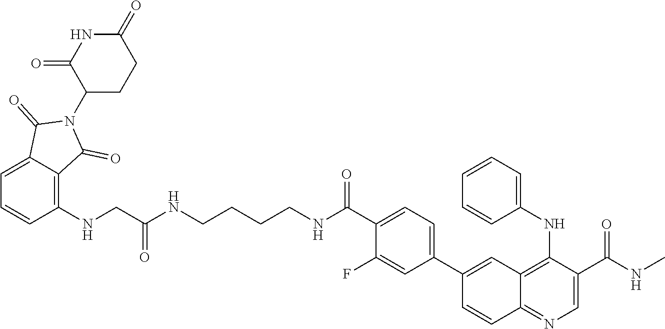 [SMILES: CNC(=O)c1cnc2ccc(-c3ccc(C(=O)NCCCCNC(=O)CNc4cccc5c4C(=O)N(C4CCC(=O)NC4=O)C5=O)c(F)c3)cc2c1Nc1ccccc1] | | +++ |
| | 6-(4-((4-(2-((2-(2,6-dioxopiperidin-3-yl)-1,3-dioxoisoindolin-4- | | |
| | yl)amino)acetamido)butyl)carbamoyl)-3-fluorophenyl)-N-methyl-4- | | |
| | (phenylamino)quinoline-3-carboxamide | | |
| 351 | 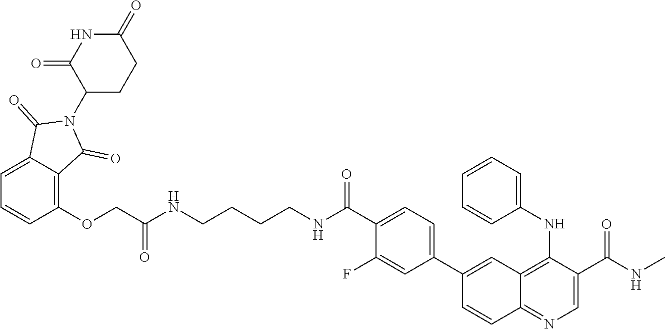 [SMILES: CNC(=O)c1cnc2ccc(-c3ccc(C(=O)NCCCCNC(=O)COc4cccc5c4C(=O)N(C4CCC(=O)NC4=O)C5=O)c(F)c3)cc2c1Nc1ccccc1] | +++ | |
| | 6-(4-((4-(2-((2-(2,6-dioxopiperidin-3-yl)-1,3-dioxoisoindolin-4- | | |
| | yl)oxy)acetamido)butyl)carbamoyl)-3-fluorophenyl)-N-methyl-4- | | |
| | (phenylamino)quinoline-3-carboxamide | | |
| 353 | 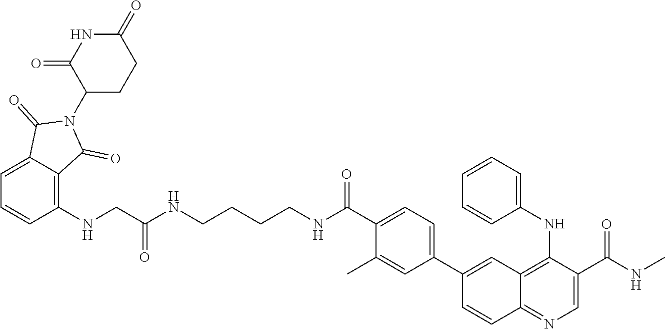 [SMILES: CNC(=O)c1cnc2ccc(-c3ccc(C(=O)NCCCCNC(=O)CNc4cccc5c4C(=O)N(C4CCC(=O)NC4=O)C5=O)c(C)c3)cc2c1Nc1ccccc1] | +++ | |
| | 6-(4-((4-(2-((2-(2,6-dioxopiperidin-3-yl)-1,3-dioxoisoindolin-4- | | |
| | yl)amino)acetamido)butyl)carbamoyl)-3-methylphenyl)-N-methyl-4- | | |
| | (phenylamino)quinoline-3-carboxamide | | |
| 354 | 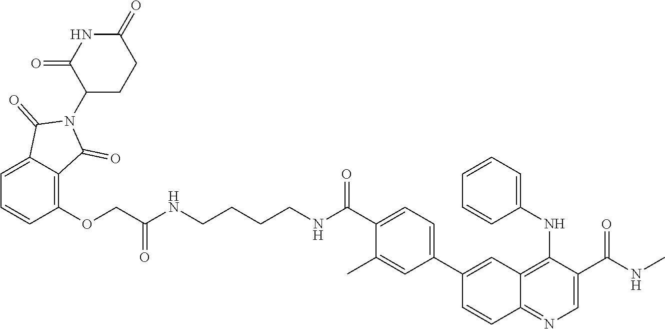 [SMILES: CNC(=O)c1cnc2ccc(-c3ccc(C(=O)NCCCCNC(=O)COc4cccc5c4C(=O)N(C4CCC(=O)NC4=O)C5=O)c(C)c3)cc2c1Nc1ccccc1] | +++ | |
| | 6-(4-((4-(2-((2-(2,6-dioxopiperidin-3-yl)-1,3-dioxoisoindolin-4- | | |
| | yl)oxy)acetamido)butyl)carbamoyl)-3-methylphenyl)-N-methyl-4- | | |
| | (phenylamino)quinoline-3-carboxamide | | |
| 356 | 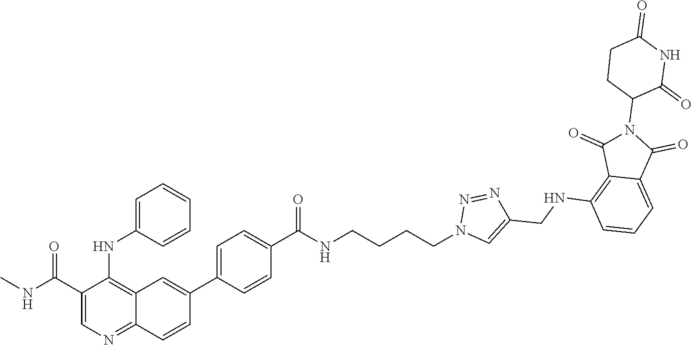 [SMILES: CNC(=O)c1cnc2ccc(-c3ccc(C(=O)NCCCCn4cc(CNc5cccc6c5C(=O)N(C5CCC(=O)NC5=O)C6=O)nn4)cc3)cc2c1Nc1ccccc1] | +++ | |
| | 6-(4-((4-(4-(((2-(2,6-dioxopiperidin-3-yl)-1,3-dioxoisoindolin-4- | | |
| | yl)amino)methyl)-1H-1,2,3-triazol-1-yl)butyl)carbamoyl)phenyl)-N- | | |
| | methyl-4-(phenylamino)quinoline-3-carboxamide | | |
| 357 | 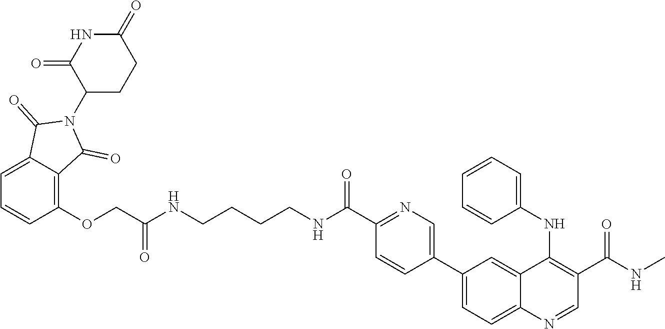 [SMILES: CNC(=O)c1cnc2ccc(-c3ccc(C(=O)NCCCCNC(=O)COc4cccc5c4C(=O)N(C4CCC(=O)NC4=O)C5=O)nc3)cc2c1Nc1ccccc1] | +++ | |
| | 6-(6-((4-(2-((2-(2,6-dioxopiperidin-3-yl)-1,3-dioxoisoindolin-4- | | |
| | yl)oxy)acetamido)butyl)carbamoyl)pyridin-3-yl)-N-methyl-4- | | |
| | (phenylamino)quinoline-3-carboxamide | | |
| 358 | 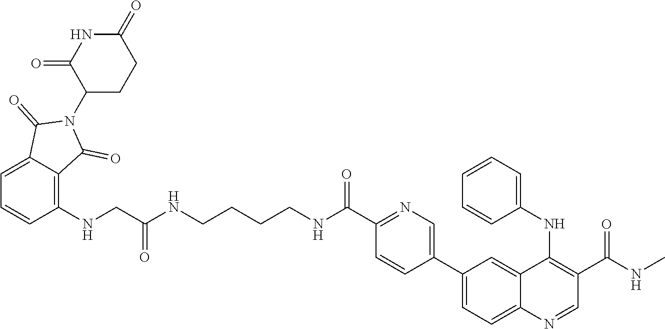 [SMILES: CNC(=O)c1cnc2ccc(-c3ccc(C(=O)NCCCCNC(=O)CNc4cccc5c4C(=O)N(C4CCC(=O)NC4=O)C5=O)nc3)cc2c1Nc1ccccc1] | +++ | |
| | 6-(6-((4-(2-((2-(2,6-dioxopiperidin-3-yl)-1,3-dioxoisoindolin-4- | | |
| | yl)amino)acetamido)butyl)carbamoyl)pyridin-3-yl)-N-methyl-4- | | |
| | (phenylamino)quinoline-3-carboxamide | | |
| 359 | 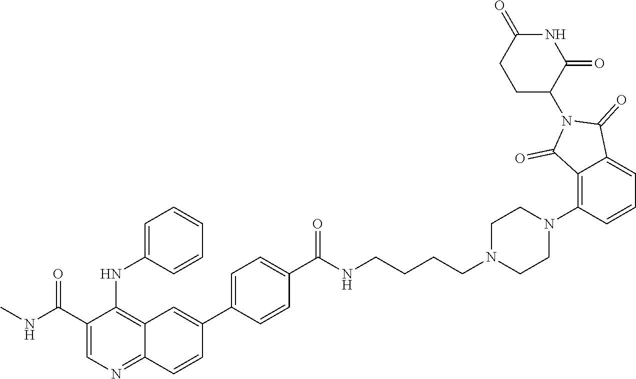 [SMILES: CNC(=O)c1cnc2ccc(-c3ccc(C(=O)NCCCCN4CCN(c5cccc6c5C(=O)N(C5CCC(=O)NC5=O)C6=O)CC4)cc3)cc2c1Nc1ccccc1] | +++ | |
| | 6-(4-((4-(4-(2-(2,6-dioxopiperidin-3-yl)-1,3-dioxoisoindolin-4- | | |
| | yl)piperazin-1-yl)butyl)carbamoyl)phenyl)-N-methyl-4- | | |
| | (phenylamino)quinoline-3-carboxamide | | |
| 360 | 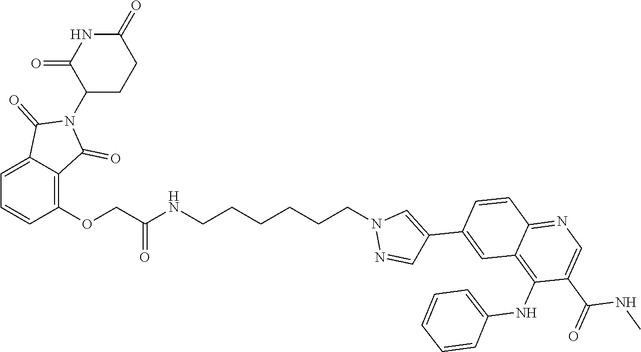 [SMILES: CNC(=O)c1cnc2ccc(-c3cnn(CCCCCCNC(=O)COc4cccc5c4C(=O)N(C4CCC(=O)NC4=O)C5=O)c3)cc2c1Nc1ccccc1] | +++ | |
| | 6-(1-(6-(2-((2-(2,6-dioxopiperidin-3-yl)-1,3-dioxoisoindolin-4- | | |
| | yl)oxy)acetamido)hexyl)-1H-pyrazol-4-yl)-N-methyl-4- | | |
| | (phenylamino)quinoline-3-carboxamide | | |
| 361 | 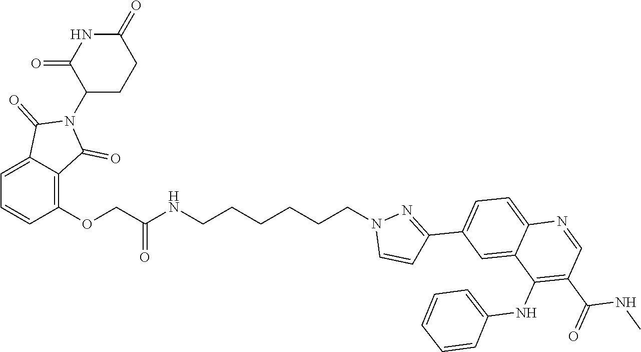 [SMILES: CNC(=O)c1cnc2ccc(-c3ccn(CCCCCCNC(=O)COc4cccc5c4C(=O)N(C4CCC(=O)NC4=O)C5=O)n3)cc2c1Nc1ccccc1] | | +++ |
| | 6-(1-(6-(2-((2-(2,6-dioxopiperidin-3-yl)-1,3-dioxoisoindolin-4- | | |
| | yl)oxy)acetamido)hexyl)-1H-pyrazol-3-yl)-N-methyl-4- | | |
| | (phenylamino)quinoline-3-carboxamide | | |
| 363 | 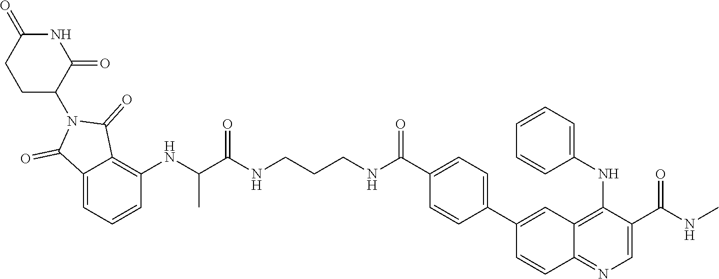 [SMILES: CNC(=O)c1cnc2ccc(-c3ccc(C(=O)NCCCNC(=O)C(C)Nc4cccc5c4C(=O)N(C4CCC(=O)NC4=O)C5=O)cc3)cc2c1Nc1ccccc1] | +++ | |
| | 6-(4-((3-(2-((2-(2,6-dioxopiperidin-3-yl)-1,3-dioxoisoindolin-4- | | |
| | yl)amino)propanamido)propyl)carbamoyl)phenyl)-N-methyl-4- | | |
| | (phenylamino)quinoline-3-carboxamide | | |
| 365 | 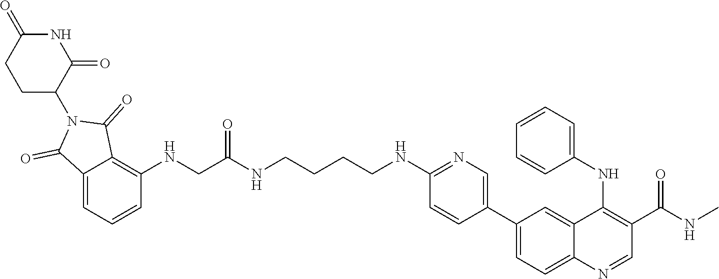 [SMILES: CNC(=O)c1cnc2ccc(-c3ccc(NCCCCNC(=O)CNc4cccc5c4C(=O)N(C4CCC(=O)NC4=O)C5=O)nc3)cc2c1Nc1ccccc1] | +++ | |
| | 6-(6-((4-(2-((2-(2,6-dioxopiperidin-3-yl)-1,3-dioxoisoindolin-4- | | |
| | yl)amino)acetamido)butyl)amino)pyridin-3-yl)-N-methyl-4- | | |
| | (phenylamino)quinoline-3-carboxamide | | |
| 366 | 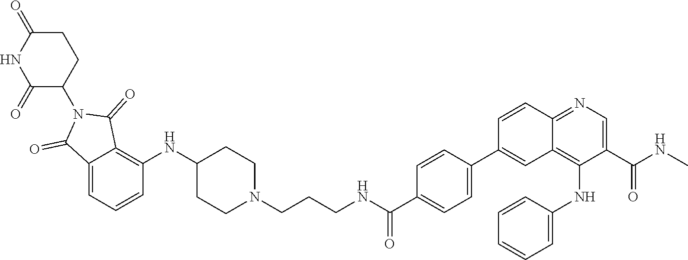 [SMILES: CNC(=O)c1cnc2ccc(-c3ccc(C(=O)NCCCN4CCC(Nc5cccc6c5C(=O)N(C5CCC(=O)NC5=O)C6=O)CC4)cc3)cc2c1Nc1ccccc1] | | +++ |
| | 6-(4-((3-(4-((2-(2,6-dioxopiperidin-3-yl)-1,3-dioxoisoindolin-4- | | |
| | yl)amino)piperidin-1-yl)propyl)carbamoyl)phenyl)-N-methyl-4- | | |
| | (phenylamino)quinoline-3-carboxamide | | |
| 367 | 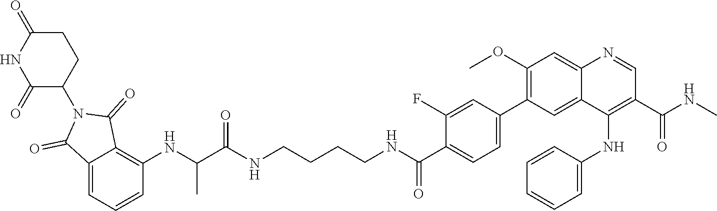 [SMILES: CNC(=O)c1cnc2cc(OC)c(-c3ccc(C(=O)NCCCCNC(=O)C(C)Nc4cccc5c4C(=O)N(C4CCC(=O)NC4=O)C5=O)c(F)c3)cc2c1Nc1ccccc1] | | +++ |
| | 6-(4-((4-(2-((2-(2,6-dioxopiperidin-3-yl)-1,3-dioxoisoindolin-4- | | |
| | yl)amino)propanamido)butyl)carbamoyl)-3-fluorophenyl)-7-methoxy-N- | | |
| | methyl-4-(phenylamino)quinoline-3-carboxamide | | |
| 369 | 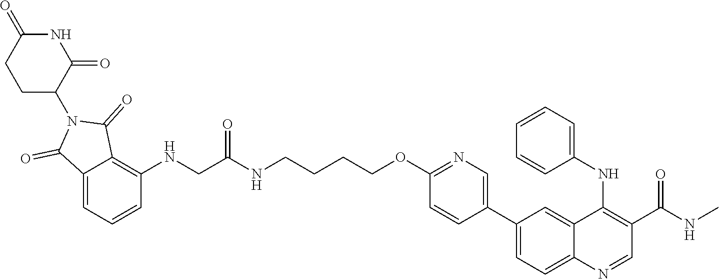 [SMILES: CNC(=O)c1cnc2ccc(-c3ccc(OCCCCNC(=O)CNc4cccc5c4C(=O)N(C4CCC(=O)NC4=O)C5=O)nc3)cc2c1Nc1ccccc1] | | +++ |
| | 6-(6-(4-(2-((2-(2,6-dioxopiperidin-3-yl)-1,3-dioxoisoindolin-4- | | |
| | yl)amino)acetamido)butoxy)pyridin-3-yl)-N-methyl-4- | | |
| | (phenylamino)quinoline-3-carboxamide | | |
| 370 | 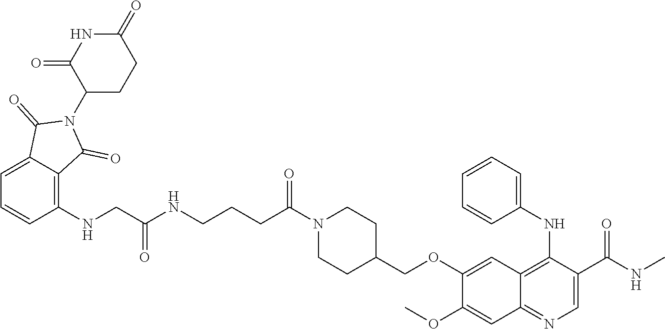 [SMILES: CNC(=O)c1cnc2cc(OC)c(OCC3CCN(C(=O)CCCNC(=O)CNc4cccc5c4C(=O)N(C4CCC(=O)NC4=O)C5=O)CC3)cc2c1Nc1ccccc1] | | +++ |
| | 6-((1-(4-(2-((2-(2,6-dioxopiperidin-3-yl)-1,3-dioxoisoindolin-4- | | |
| | yl)amino)acetamido)butanoyl)piperidin-4-yl)methoxy)-7-methoxy-N- | | |
| | methyl-4-(phenylamino)quinoline-3-carboxamide | | |
| 371 | 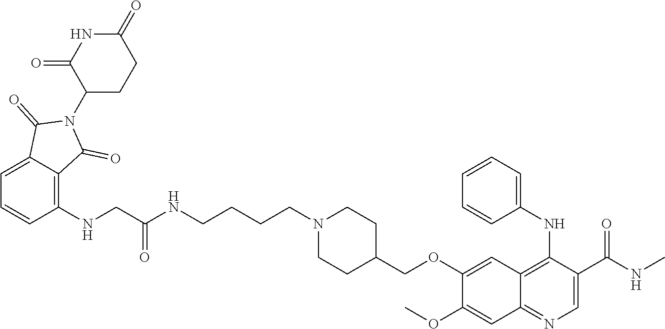 [SMILES: CNC(=O)c1cnc2cc(OC)c(OCC3CCN(CCCCNC(=O)CNc4cccc5c4C(=O)N(C4CCC(=O)NC4=O)C5=O)CC3)cc2c1Nc1ccccc1] | | +++ |
| | 6-((1-(4-(2-((2-(2,6-dioxopiperidin-3-yl)-1,3-dioxoisoindolin-4- | | |
| | yl)amino)acetamido)butyl)piperidin-4-yl)methoxy)-7-methoxy-N- | | |
| | methyl-4-(phenylamino)quinoline-3-carboxamide | | |
| 372 | 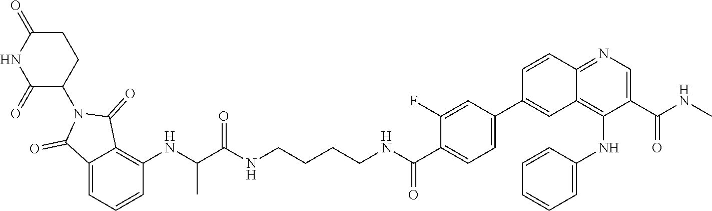 [SMILES: CNC(=O)c1cnc2ccc(-c3ccc(C(=O)NCCCCNC(=O)C(C)Nc4cccc5c4C(=O)N(C4CCC(=O)NC4=O)C5=O)c(F)c3)cc2c1Nc1ccccc1] | +++ | |
| | 6-(4-((4-(2-((2-(2,6-dioxopiperidin-3-yl)-1,3-dioxoisoindolin-4- | | |
| | yl)amino)propanamido)butyl)carbamoyl)-3-fluorophenyl)-N-methyl-4- | | |
| | (phenylamino)quinoline-3-carboxamide | | |
| 373 | 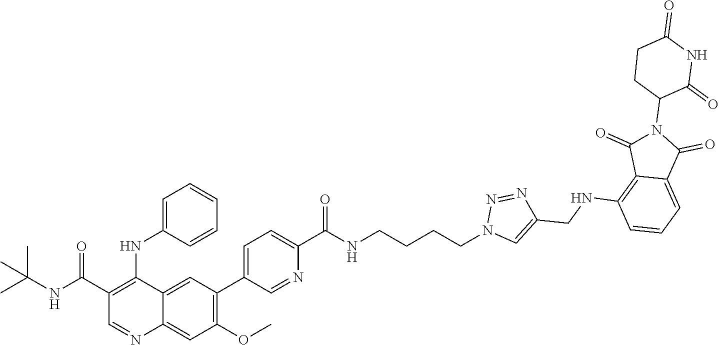 [SMILES: COc1cc2ncc(C(=O)NC(C)(C)C)c(Nc3ccccc3)c2cc1-c1ccc(C(=O)NCCCCn2cc(CNc3cccc4c3C(=O)N(C3CCC(=O)NC3=O)C4=O)nn2)nc1] | | +++ |
| | N-(tert-butyl)-6-(6-((4-(4-(((2-(2,6-dioxopiperidin-3-yl)-1,3- | | |
| | dioxoisoindolin-4-yl)amino)methyl)-1H-1,2,3-triazol-1- | | |
| | yl)butyl)carbamoyl)pyridin-3-yl)-7-methoxy-4-(phenylamino)quinoline- | | |
| | 3-carboxamide | | |
| 374 | 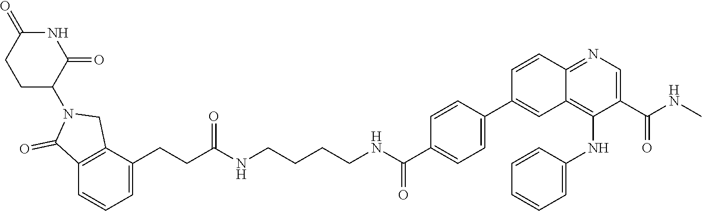 [SMILES: CNC(=O)c1cnc2ccc(-c3ccc(C(=O)NCCCCNC(=O)CCc4cccc5c4CN(C4CCC(=O)NC4=O)C5=O)cc3)cc2c1Nc1ccccc1] | +++ | |
| | 6-(4-((4-(3-(2-(2,6-dioxopiperidin-3-yl)-1-oxoisoindolin-4- | | |
| | yl)propanamido)butyl)carbamoyl)phenyl)-N-methyl-4- | | |
| | (phenylamino)quinoline-3-carboxamide | | |
| 375 | 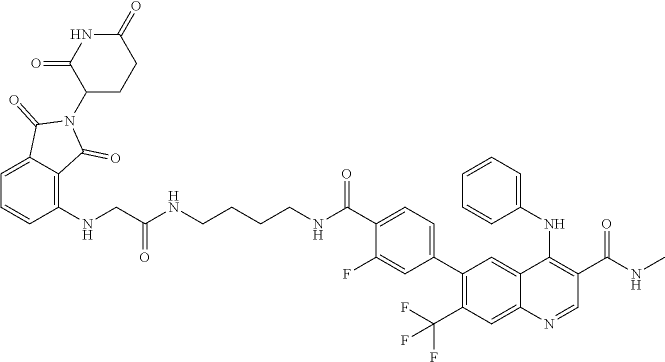 [SMILES: CNC(=O)c1cnc2cc(C(F)(F)F)c(-c3ccc(C(=O)NCCCCNC(=O)CNc4cccc5c4C(=O)N(C4CCC(=O)NC4=O)C5=O)c(F)c3)cc2c1Nc1ccccc1] | +++ | |
| | 6-(4-((4-(2-((2-(2,6-dioxopiperidin-3-yl)-1,3-dioxoisoindolin-4- | | |
| | yl)amino)acetamido)butyl)carbamoyl)-3-fluorophenyl)-N-methyl-4- | | |
| | (phenylamino)-7-(trifluoromethyl)quinoline-3-carboxamide | | |
| 376 | 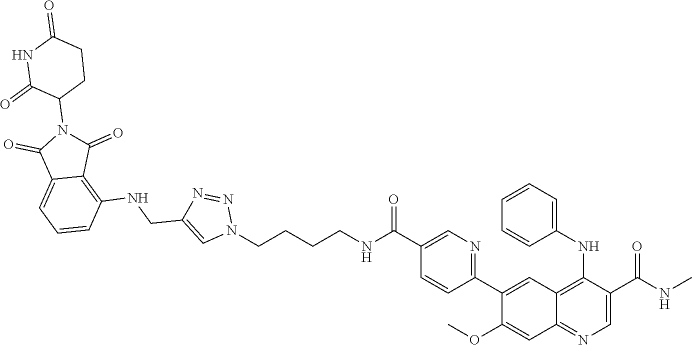 [SMILES: CNC(=O)c1cnc2cc(OC)c(-c3ccc(C(=O)NCCCCn4cc(CNc5cccc6c5C(=O)N(C5CCC(=O)NC5=O)C6=O)nn4)cn3)cc2c1Nc1ccccc1] | +++ | |
| | 6-(4-((2-(4-((2-(2,6-dioxopiperidin-3-yl)-1,3-dioxoisoindolin-4- | | |
| | yl)amino)piperidin-1-yl)ethyl)carbamoyl)phenyl)-N-methyl-4- | | |
| | (phenylamino)quinoline-3-carboxamide | | |
| 378 | 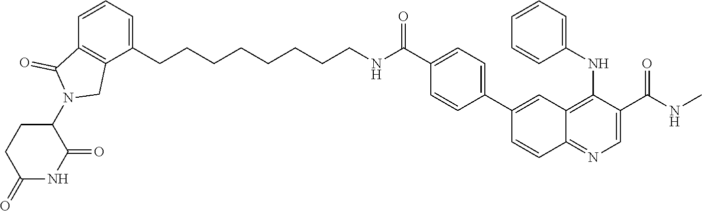 [SMILES: CNC(=O)c1cnc2ccc(-c3ccc(C(=O)NCCCCCCCCc4cccc5c4CN(C4CCC(=O)NC4=O)C5=O)cc3)cc2c1Nc1ccccc1] | +++ | |
| | 6-(4-((8-(2-(2,6-dioxopiperidin-3-yl)-1-oxoisoindolin-4- | | |
| | yl)octyl)carbamoyl)phenyl)-N-methyl-4-(phenylamino)quinoline-3- | | |
| | carboxamide | | |
| 379 | 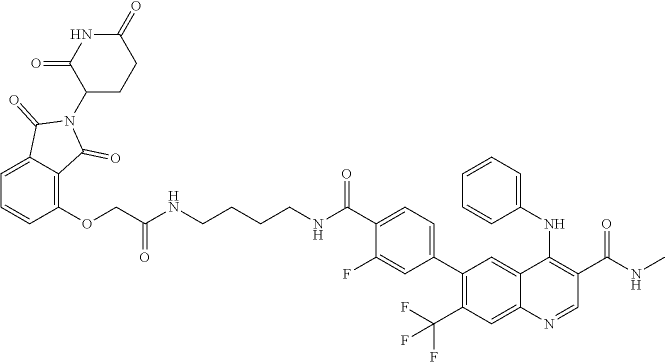 [SMILES: CNC(=O)c1cnc2cc(C(F)(F)F)c(-c3ccc(C(=O)NCCCCNC(=O)COc4cccc5c4C(=O)N(C4CCC(=O)NC4=O)C5=O)c(F)c3)cc2c1Nc1ccccc1] | +++ | |
| | 6-(4-((4-(2-((2-(2,6-dioxopiperidin-3-yl)-1,3-dioxoisoindolin-4- | | |
| | yl)oxy)acetamido)butyl)carbamoyl)-3-fluorophenyl)-N-methyl-4- | | |
| | (phenylamino)-7-(trifluoromethyl)quinoline-3-carboxamide | | |
| 380 | 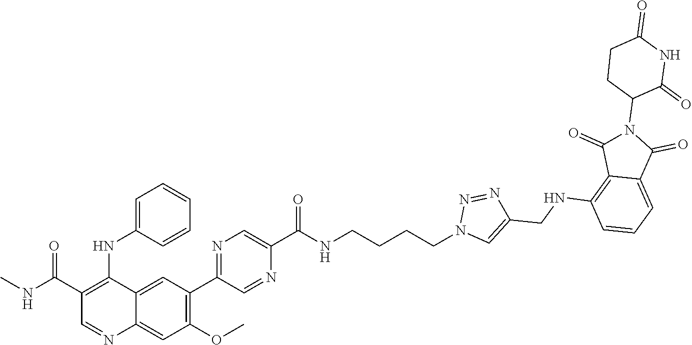 [SMILES: CNC(=O)c1cnc2cc(OC)c(-c3cnc(C(=O)NCCCCn4cc(CNc5cccc6c5C(=O)N(C5CCC(=O)NC5=O)C6=O)nn4)cn3)cc2c1Nc1ccccc1] | | +++ |
| | 6-(5-((4-(4-(((2-(2,6-dioxopiperidin-3-yl)-1,3-dioxoisoindolin-4- | | |
| | yl)amino)methyl)-1H-1,2,3-triazol-1-yl)butyl)carbamoyl)pyrazin-2-yl)- | | |
| | 7-methoxy-N-methyl-4-(phenylamino)quinoline-3-carboxamide | | |
| 381 | 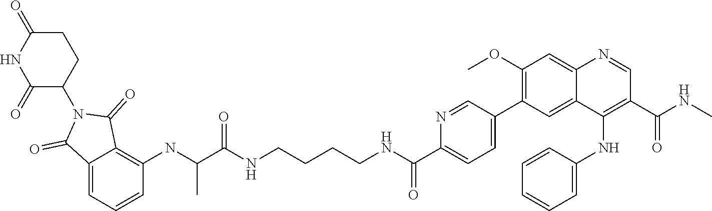 [SMILES: CNC(=O)c1cnc2cc(OC)c(-c3ccc(C(=O)NCCCCNC(=O)C(C)Nc4cccc5c4C(=O)N(C4CCC(=O)NC4=O)C5=O)nc3)cc2c1Nc1ccccc1] | +++ | |
| | 6-(6-((4-(2-((2-(2,6-dioxopiperidin-3-yl)-1,3-dioxoisoindolin-4- | | |
| | yl)oxy)propanamido)butyl)carbamoyl)pyridin-3-yl)-7-methoxy-N- | | |
| | methyl-4-(phenylamino)quinoline-3-carboxamide | | |
| 382 | 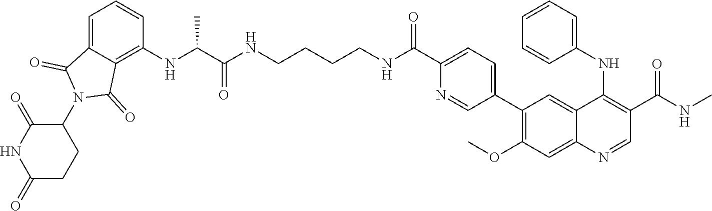 [SMILES: CNC(=O)c1cnc2cc(OC)c(-c3ccc(C(=O)NCCCCNC(=O)[C@@H](C)Nc4cccc5c4C(=O)N(C4CCC(=O)NC4=O)C5=O)nc3)cc2c1Nc1ccccc1] | +++ | |
| | 6-(6-((4-((2R)-2-((2-(2,6-dioxopiperidin-3-yl)-1,3-dioxoisoindolin-4- | | |
| | yl)amino)propanamido)butyl)carbamoyl)pyridin-3-yl)-7-methoxy-N- | | |
| | methyl-4-(phenylamino)quinoline-3-carboxamide | | |
| 384 | 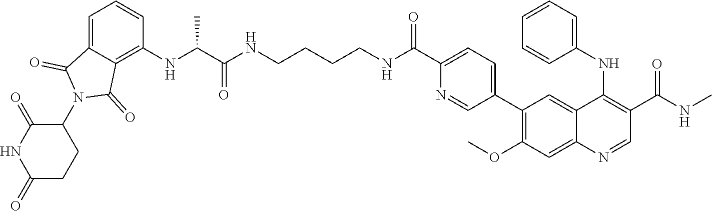 [SMILES: CNC(=O)c1cnc2cc(OC)c(-c3ccc(C(=O)NCCCCNC(=O)[C@@H](C)Nc4cccc5c4C(=O)N(C4CCC(=O)NC4=O)C5=O)nc3)cc2c1Nc1ccccc1] | +++ | |
| | 6-(6-((4-((2R)-2-((2-(2,6-dioxopiperidin-3-yl)-1,3-dioxoisoindolin-4- | | |
| | yl)amino)propanamido)butyl)carbamoyl)pyridin-3-yl)-7-methoxy-N- | | |
| | methyl-4-(phenylamino)quinoline-3-carboxamide | | |
| 385 | 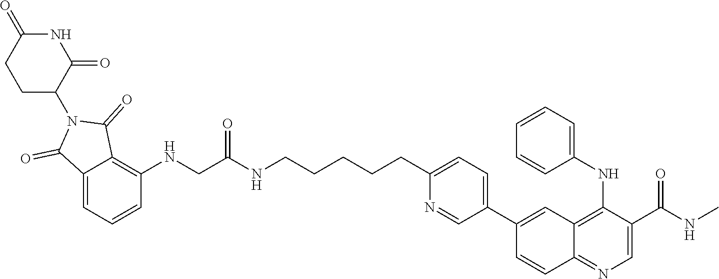 [SMILES: CNC(=O)c1cnc2ccc(-c3ccc(CCCCCNC(=O)CNc4cccc5c4C(=O)N(C4CCC(=O)NC4=O)C5=O)nc3)cc2c1Nc1ccccc1] | +++ | |
| | 6-(6-(5-(2-((2-(2,6-dioxopiperidin-3-yl)-1,3-dioxoisoindolin-4- | | |
| | yl)amino)acetamido)pentyl)pyridin-3-yl)-N-methyl-4- | | |
| | (phenylamino)quinoline-3-carboxamide | | |
| 386 | 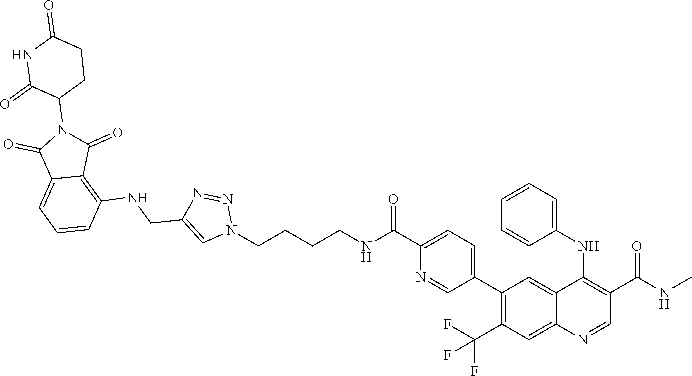 [SMILES: CNC(=O)c1cnc2cc(C(F)(F)F)c(-c3ccc(C(=O)NCCCCn4cc(CNc5cccc6c5C(=O)N(C5CCC(=O)NC5=O)C6=O)nn4)nc3)cc2c1Nc1ccccc1] | +++ | |
| | 6-(6-((4-(4-(((2-(2,6-dioxopiperidin-3-yl)-1,3-dioxoisoindolin-4- | | |
| | yl)amino)methyl)-1H-1,2,3-triazol-1-yl)butyl)carbamoyl)pyridin-3-yl)- | | |
| | N-methyl-4-(phenylamino)-7-(trifluoromethyl)quinoline-3-carboxamide | | |
| 387 | 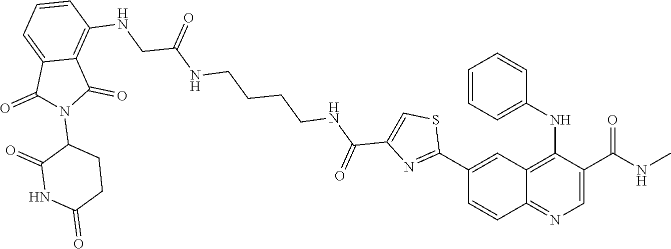 [SMILES: CNC(=O)c1cnc2ccc(-c3nc(C(=O)NCCCCNC(=O)CNc4cccc5c4C(=O)N(C4CCC(=O)NC4=O)C5=O)cs3)cc2c1Nc1ccccc1] | +++ | |
| | N-(4-(2-((2-(2,6-dioxopiperidin-3-yl)-1,3-dioxoisoindolin-4- | | |
| | yl)amino)acetamido)butyl)-2-(3-(methylcarbamoyl)-4- | | |
| | (phenylamino)quinolin-6-yl)thiazole-4-carboxamide | | |
| 388 | 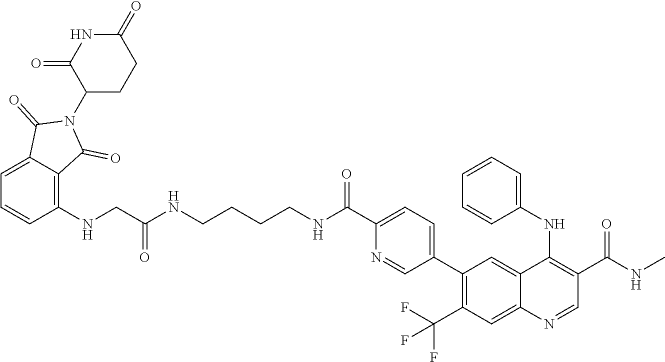 [SMILES: CNC(=O)c1cnc2cc(C(F)(F)F)c(-c3ccc(C(=O)NCCCCNC(=O)CNc4cccc5c4C(=O)N(C4CCC(=O)NC4=O)C5=O)nc3)cc2c1Nc1ccccc1] | +++ | |
| | 6-(6-((4-(2-((2-(2,6-dioxopiperidin-3-yl)-1,3-dioxoisoindolin-4- | | |
| | yl)amino)acetamido)butyl)carbamoyl)pyridin-3-yl)-N-methyl-4- | | |
| | (phenylamino)-7-(trifluoromethyl)quinoline-3-carboxamide | | |
| 389 | 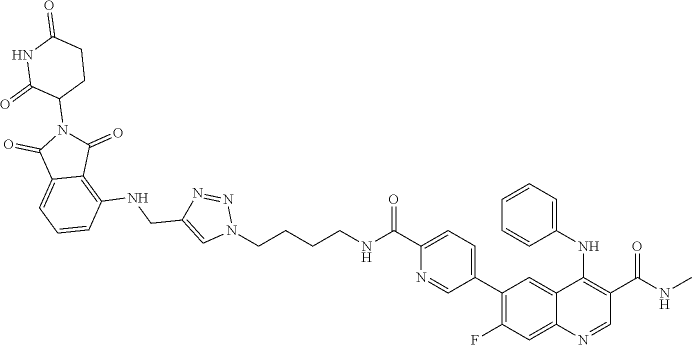 [SMILES: CNC(=O)c1cnc2cc(F)c(-c3ccc(C(=O)NCCCCn4cc(CNc5cccc6c5C(=O)N(C5CCC(=O)NC5=O)C6=O)nn4)nc3)cc2c1Nc1ccccc1] | +++ | |
| | 6-(6-((4-(4-(((2-(2,6-dioxopiperidin-3-yl)-1,3-dioxoisoindolin-4- | | |
| | yl)amino)methyl)-1H-1,2,3-triazol-1-yl)butyl)carbamoyl)pyridin-3-yl)-7- | | |
| | fluoro-N-methyl-4-(phenylamino)quinoline-3-carboxamide | | |
| 395 | 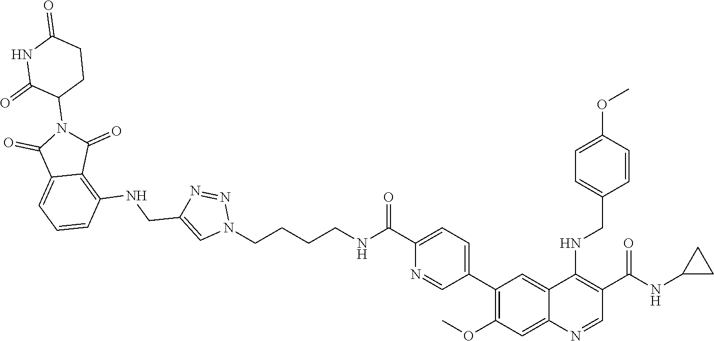 [SMILES: COc1ccc(CNc2c(C(=O)NC3CC3)cnc3cc(OC)c(-c4ccc(C(=O)NCCCCn5cc(CNc6cccc7c6C(=O)N(C6CCC(=O)NC6=O)C7=O)nn5)nc4)cc23)cc1] | +++ | |
| | N-cyclopropyl-6-(6-((4-(4-(((2-(2,6-dioxopiperidin-3-yl)-1,3- | | |
| | dioxoisoindolin-4-yl)amino)methyl)-1H-1,2,3-triazol-1- | | |
| | yl)butyl)carbamoyl)pyridin-3-yl)-7-methoxy-4-((4- | | |
| | methoxybenzyl)amino)quinoline-3-carboxamide | | |
| 396 | 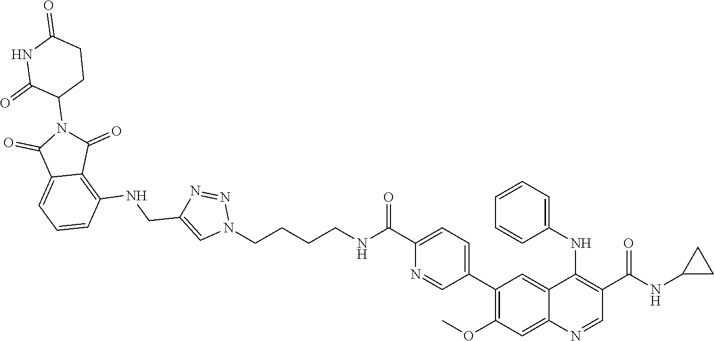 [SMILES: COc1cc2ncc(C(=O)NC3CC3)c(Nc3ccccc3)c2cc1-c1ccc(C(=O)NCCCCn2cc(CNc3cccc4c3C(=O)N(C3CCC(=O)NC3=O)C4=O)nn2)nc1] | +++ | |
| | N-cyclopropyl-6-(6-((4-(4-(((2-(2,6-dioxopiperidin-3-yl)-1,3- | | |
| | dioxoisoindolin-4-yl)amino)methyl)-1H-1,2,3-triazol-1- | | |
| | yl)butyl)carbamoyl)pyridin-3-yl)-7-methoxy-4-(phenylamino)quinoline- | | |
| | 3-carboxamide | | |
| 397 | 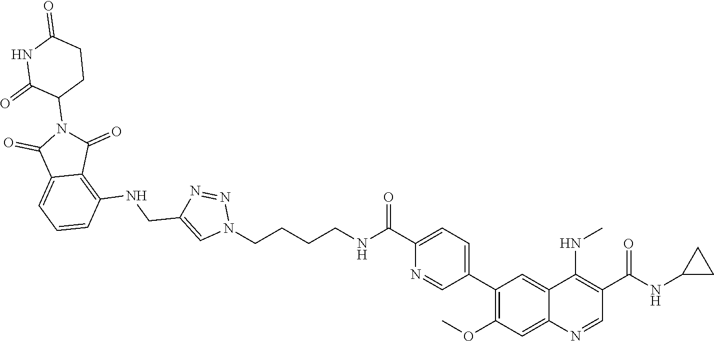 [SMILES: CNc1c(C(=O)NC2CC2)cnc2cc(OC)c(-c3ccc(C(=O)NCCCCn4cc(CNc5cccc6c5C(=O)N(C5CCC(=O)NC5=O)C6=O)nn4)nc3)cc12] | +++ | |
| | N-cyclopropyl-6-(6-((4-(4-(((2-(2,6-dioxopiperidin-3-yl)-1,3- | | |
| | dioxoisoindolin-4-yl)amino)methyl)-1H-1,2,3-triazol-1- | | |
| | yl)butyl)carbamoyl)pyridin-3-yl)-7-methoxy-4-(methylamino)quinoline- | | |
| | 3-carboxamide | | |
| 398 | 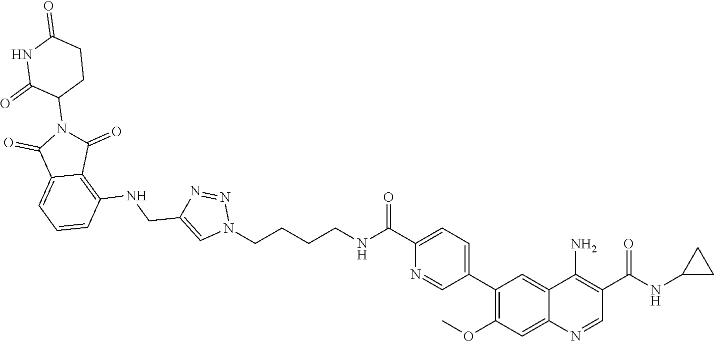 [SMILES: COc1cc2ncc(C(=O)NC3CC3)c(N)c2cc1-c1ccc(C(=O)NCCCCn2cc(CNc3cccc4c3C(=O)N(C3CCC(=O)NC3=O)C4=O)nn2)nc1] | +++ | |
| | 4-amino-N-cyclopropyl-6-(6-((4-(4-(((2-(2,6-dioxopiperidin-3-yl)-1,3- | | |
| | dioxoisoindolin-4-yl)amino)methyl)-1H-1,2,3-triazol-1- | | |
| | yl)butyl)carbamoyl)pyridin-3-yl)-7-methoxyquinoline-3-carboxamide | | |
| 401 | 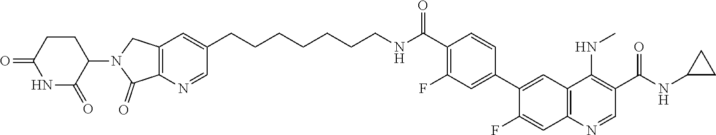 [SMILES: CNc1c(C(=O)NC2CC2)cnc2cc(F)c(-c3ccc(C(=O)NCCCCCCCc4cnc5c(c4)CN(C4CCC(=O)NC4=O)C5=O)c(F)c3)cc12] | +++ | |
| | N-cyclopropyl-6-(4-((7-(6-(2,6-dioxopiperidin-3-y1)-7-oxo-6,7-dihydro- | | |
| | 5H-pyrrolo[3,4-b]pyridin-3-yl)heptyl)carbamoyl)-3-fluorophenyl)-7- | | |
| | fluoro-4-(methylamino)quinoline-3-carboxamide | | |
| 404 | 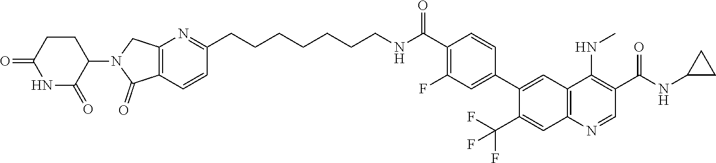 [SMILES: CNc1c(C(=O)NC2CC2)cnc2cc(C(F)(F)F)c(-c3ccc(C(=O)NCCCCCCCc4ccc5c(n4)CN(C4CCC(=O)NC4=O)C5=O)c(F)c3)cc12] | +++ | |
| | N-cyclopropyl-6-(4-((7-(6-(2,6-dioxopiperidin-3-yl)-5-oxo-6,7-dihydro- | | |
| | 5H-pyrrolo[3,4-b]pyridin-2-yl)heptyl)carbamoyl)-3-fluorophenyl)-4- | | |
| | (methylamino)-7-(trifluoromethyl)quinoline-3-carboxamide | | |
| 405 | 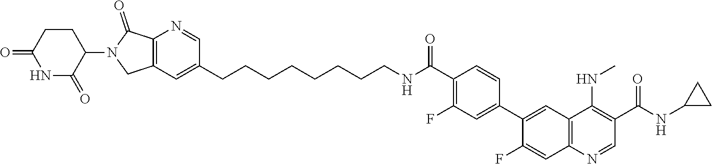 [SMILES: CNc1c(C(=O)NC2CC2)cnc2cc(F)c(-c3ccc(C(=O)NCCCCCCCCc4cnc5c(c4)CN(C4CCC(=O)NC4=O)C5=O)c(F)c3)cc12] | +++ | |
| | N-cyclopropyl-6-(4-((8-(6-(2,6-dioxopiperidin-3-yl)-7-oxo-6,7-dihydro- | | |
| | 5H-pyrrolo[3,4-b]pyridin-3-yl)octyl)carbamoyl)-3-fluorophenyl)-7- | | |
| | fluoro-4-(methylamino)quinoline-3-carboxamide | | |
| 408 | 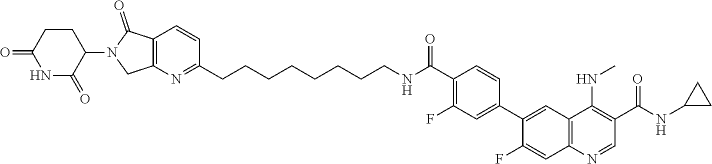 [SMILES: CNc1c(C(=O)NC2CC2)cnc2cc(F)c(-c3ccc(C(=O)NCCCCCCCCc4ccc5c(n4)CN(C4CCC(=O)NC4=O)C5=O)c(F)c3)cc12] | +++ | |
| | N-cyclopropyl-6-(4-((8-(6-(2,6-dioxopiperidin-3-yl)-5-oxo-6,7-dihydro- | | |
| | 5H-pyrrolo[3,4-b]pyridin-2-yl)octyl)carbamoyl)-3-fluorophenyl)-7- | | |
| | fluoro-4-(methylamino)quinoline-3-carboxamide | | |
| 409 | 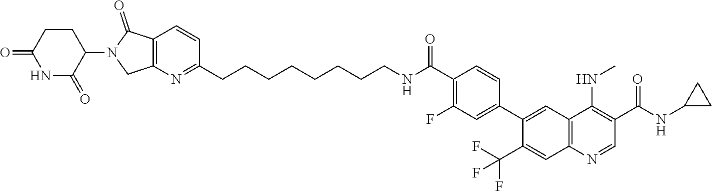 [SMILES: CNc1c(C(=O)NC2CC2)cnc2cc(C(F)(F)F)c(-c3ccc(C(=O)NCCCCCCCCc4ccc5c(n4)CN(C4CCC(=O)NC4=O)C5=O)c(F)c3)cc12] | +++ | |
| | N-cyclopropyl-6-(4-((8-(6-(2,6-dioxopiperidin-3-yl)-5-oxo-6,7-dihydro- | | |
| | 5H-pyrrolo[3,4-b]pyridin-2-yl)octyl)carbamoyl)-3-fluorophenyl)-4- | | |
| | (methylamino)-7-(trifluoromethyl)quinoline-3-carboxamide | | |
| 411 | 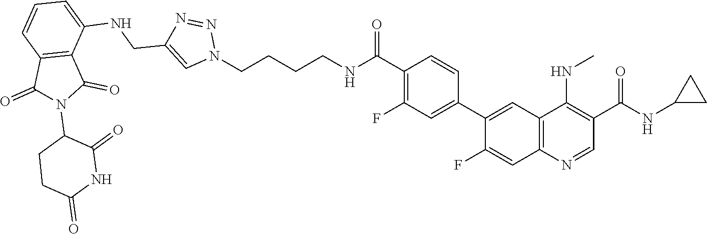 [SMILES: CNc1c(C(=O)NC2CC2)cnc2cc(F)c(-c3ccc(C(=O)NCCCCn4cc(CNc5cccc6c5C(=O)N(C5CCC(=O)NC5=O)C6=O)nn4)c(F)c3)cc12] | +++ | |
| | N-cyclopropyl-6-(4-((4-(4-(((2-(2,6-dioxopiperidin-3-yl)-1,3- | | |
| | dioxoisoindolin-4-yl)amino)methyl)-1H-1,2,3-triazol-1- | | |
| | yl)butyl)carbamoyl)-3-fluorophenyl)-7-fluoro-4- | | |
| | (methylamino)quinoline-3-carboxamide | | |
| 412 | 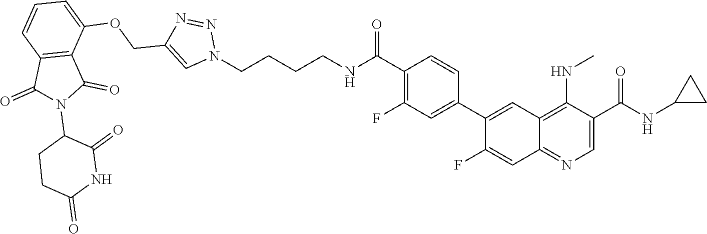 [SMILES: CNc1c(C(=O)NC2CC2)cnc2cc(F)c(-c3ccc(C(=O)NCCCCn4cc(COc5cccc6c5C(=O)N(C5CCC(=O)NC5=O)C6=O)nn4)c(F)c3)cc12] | +++ | |
| | N-cyclopropyl-6-(4-((4-(4-(((2-(2,6-dioxopiperidin-3-yl)-1,3- | | |
| | dioxoisoindolin-4-yl)oxy)methyl)-1H-1,2,3-triazol-1- | | |
| | yl)butyl)carbamoyl)-3-fluorophenyl)-7-fluoro-4- | | |
| | (methylamino)quinoline-3-carboxamide | | |
| 413 | 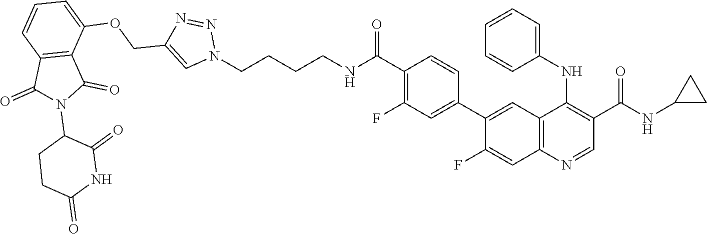 [SMILES: O=C1CCC(N2C(=O)c3cccc(OCc4cn(CCCCNC(=O)c5ccc(-c6cc7c(Nc8ccccc8)c(C(=O)NC8CC8)cnc7cc6F)cc5F)nn4)c3C2=O)C(=O)N1] | +++ | |
| | N-cyclopropyl-6-(4-((4-(4-(((2-(2,6-dioxopiperidin-3-yl)-1,3- | | |
| | dioxoisoindolin-4-yl)oxy)methyl)-1H-1,2,3-triazol-1- | | |
| | yl)butyl)carbamoyl)-3-fluorophenyl)-7-fluoro-4- | | |
| | (phenylamino)quinoline-3-carboxamide | | |
| 414 | 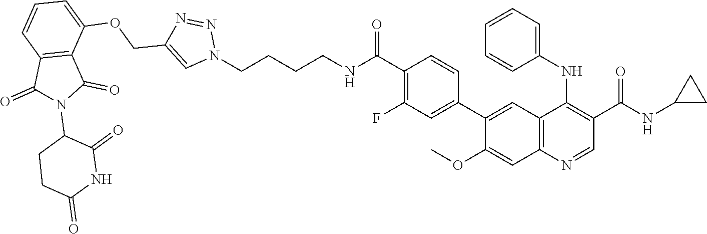 [SMILES: COc1cc2ncc(C(=O)NC3CC3)c(Nc3ccccc3)c2cc1-c1ccc(C(=O)NCCCCn2cc(COc3cccc4c3C(=O)N(C3CCC(=O)NC3=O)C4=O)nn2)c(F)c1] | +++ | |
| | N-cyclopropyl-6-(4-((4-(4-(((2-(2,6-dioxopiperidin-3-yl)-1,3- | | |
| | dioxoisoindolin-4-yl)oxy)methyl)-1H-1,2,3-triazol-1- | | |
| | yl)butyl)carbamoyl)-3-fluorophenyl)-7-methoxy-4- | | |
| | (phenylamino)quinoline-3-carboxamide | | |
| 415 | 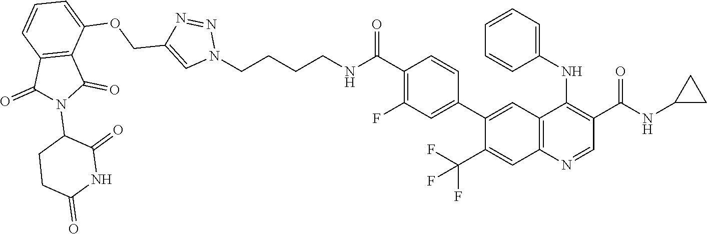 [SMILES: O=C1CCC(N2C(=O)c3cccc(OCc4cn(CCCCNC(=O)c5ccc(-c6cc7c(Nc8ccccc8)c(C(=O)NC8CC8)cnc7cc6C(F)(F)F)cc5F)nn4)c3C2=O)C(=O)N1] | +++ | |
| | N-cyclopropyl-6-(4-((4-(4-(((2-(2,6-dioxopiperidin-3-yl)-1,3 - | | |
| | dioxoisoindolin-4-yl)oxy)methyl)-1H-1,2,3-triazol-1- | | |
| | yl)butyl)carbamoyl)-3-fluorophenyl)-4-(phenylamino)-7- | | |
| | (trifluoromethyl)quinoline-3-carboxamide | | |
| 419 | 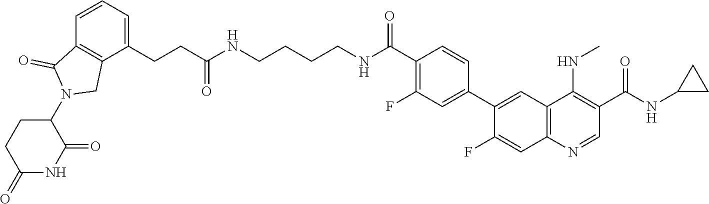 [SMILES: CNc1c(C(=O)NC2CC2)cnc2cc(F)c(-c3ccc(C(=O)NCCCCNC(=O)CCc4cccc5c4CN(C4CCC(=O)NC4=O)C5=O)c(F)c3)cc12] | +++ | |
| | N-cyclopropyl-6-(4-((4-(3-(2-(2,6-dioxopiperidin-3-yl)-1-oxoisoindolin- | | |
| | 4-yl)propanamido)butyl)carbamoyl)-3-fluorophenyl)-7-fluoro-4- | | |
| | (methylamino)quinoline-3-carboxamide | | |
| 421 | 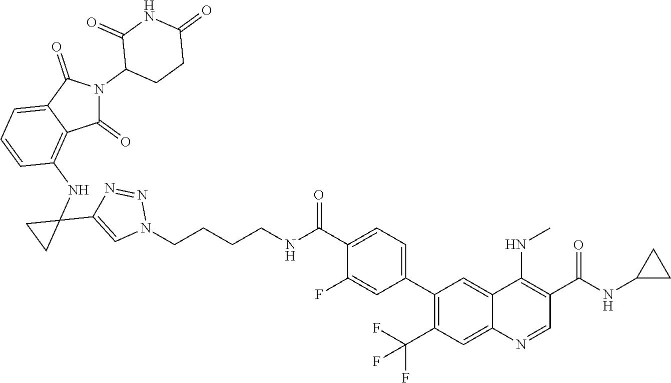 [SMILES: CNc1c(C(=O)NC2CC2)cnc2cc(C(F)(F)F)c(-c3ccc(C(=O)NCCCCn4cc(C5(Nc6cccc7c6C(=O)N(C6CCC(=O)NC6=O)C7=O)CC5)nn4)c(F)c3)cc12] | +++ | |
| | N-cyclopropyl-6-(4-((4-(4-(1-((2-(2,6-dioxopiperidin-3-yl)-1,3- | | |
| | dioxoisoindolin-4-yl)amino)cyclopropyl)-1H-1,2,3-triazol-1- | | |
| | yl)butyl)carbamoyl)-3-fluorophenyl)-4-(methylamino)-7- | | |
| | (trifluoromethyl)quinoline-3-carboxamide | | |
| 424 | 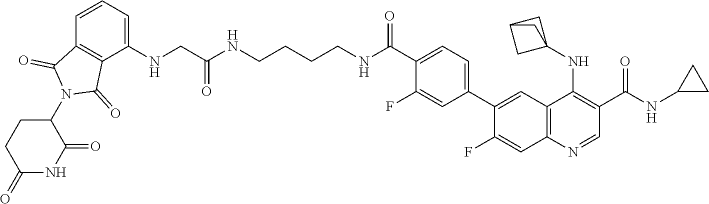 [SMILES: O=C(CNc1cccc2c1C(=O)N(C1CCC(=O)NC1=O)C2=O)NCCCCNC(=O)c1ccc(-c2cc3c(NC45CC(C4)C5)c(C(=O)NC4CC4)cnc3cc2F)cc1F] | +++ | |
| | 4-(bicyclo[1.1.1]pentan-1-ylamino)-N-cyclopropyl-6-(4-((4-(2-((2-(2,6- | | |
| | dioxopiperidin-3-yl)-1,3-dioxoisoindolin-4- | | |
| | yl)amino)acetamido)butyl)carbamoyl)-3-fluorophenyl)-7- | | |
| | fluoroquinoline-3-carboxamide | | |
| 429 | 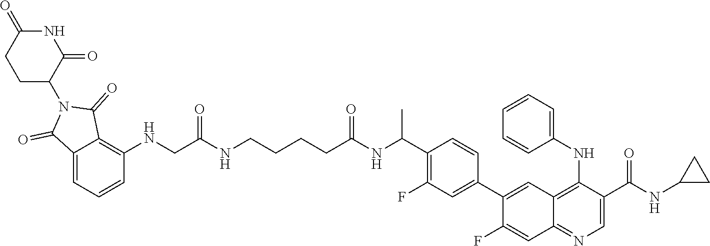 [SMILES: CC(NC(=O)CCCCNC(=O)CNc1cccc2c1C(=O)N(C1CCC(=O)NC1=O)C2=O)c1ccc(-c2cc3c(Nc4ccccc4)c(C(=O)NC4CC4)cnc3cc2F)cc1F] | +++ | |
| | N-cyclopropyl-6-(4-(1-(5-(2-((2-(2,6-dioxopiperidin-3-yl)-1,3- | | |
| | dioxoisoindolin-4-yl)amino)acetamido)pentanamido)ethyl)-3- | | |
| | fluorophenyl)-7-fluoro-4-(phenylamino)quinoline-3-carboxamide | | |
| 430 | 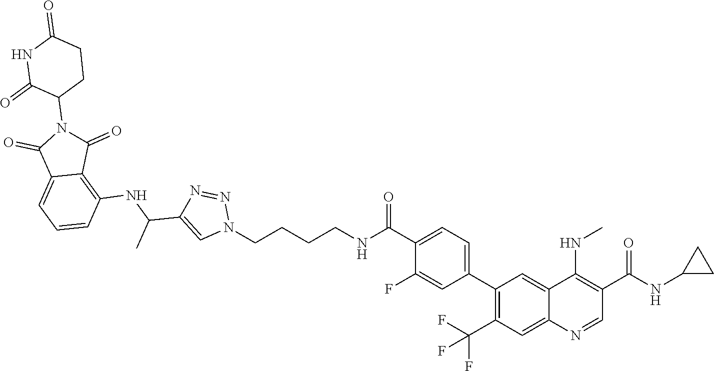 [SMILES: CNc1c(C(=O)NC2CC2)cnc2cc(C(F)(F)F)c(-c3ccc(C(=O)NCCCCn4cc(C(C)Nc5cccc6c5C(=O)N(C5CCC(=O)NC5=O)C6=O)nn4)c(F)c3)cc12] | +++ | |
| | N-cyclopropyl-6-(4-((4-(4-(1-((2-(2,6-dioxopiperidin-3-yl)-1,3- | | |
| | dioxoisoindolin-4-yl)amino)ethyl)-1H-1,2,3-triazol-1- | | |
| | yl)butyl)carbamoyl)-3-fluorophenyl)-4-(methylamino)-7- | | |
| | (trifluoromethyl)quinoline-3-carboxamide | | |
| 431 | 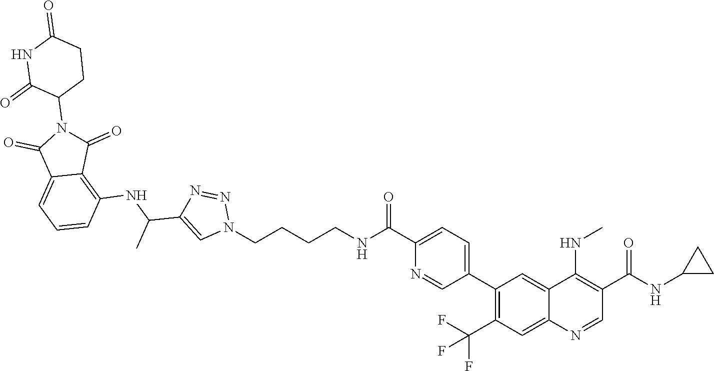 [SMILES: CNc1c(C(=O)NC2CC2)cnc2cc(C(F)(F)F)c(-c3ccc(C(=O)NCCCCn4cc(C(C)Nc5cccc6c5C(=O)N(C5CCC(=O)NC5=O)C6=O)nn4)nc3)cc12] | ++ | |
| | N-cyclopropyl-6-(6-((4-(4-(1-((2-(2,6-dioxopiperidin-3-yl)-1,3- | | |
| | dioxoisoindolin-4-yl)amino)ethyl)-1H-1,2,3-triazol-1- | | |
| | yl)butyl)carbamoyl)pyridin-3-yl)-4-(methylamino)-7- | | |
| | (trifluoromethyl)quinoline-3-carboxamide | | |
| 432 | 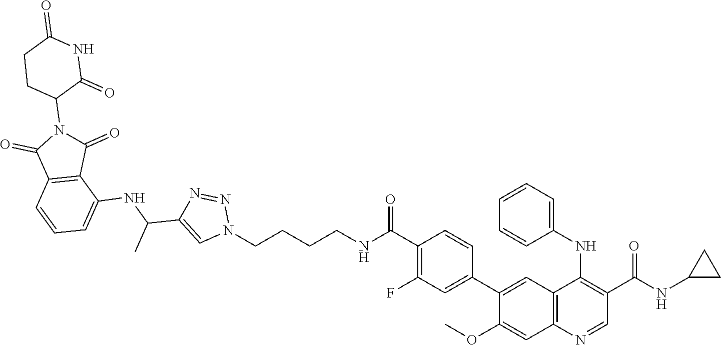 [SMILES: COc1cc2ncc(C(=O)NC3CC3)c(Nc3ccccc3)c2cc1-c1ccc(C(=O)NCCCCn2cc(C(C)Nc3cccc4c3C(=O)N(C3CCC(=O)NC3=O)C4=O)nn2)c(F)c1] | +++ | |
| | N-cyclopropyl-6-(4-((4-(4-(1-((2-(2,6-dioxopiperidin-3-yl)-1,3- | | |
| | dioxoisoindolin-4-yl)amino)ethyl)-1H-1,2,3-triazol-1- | | |
| | yl)butyl)carbamoyl)-3-fluorophenyl)-7-methoxy-4- | | |
| | (phenylamino)quinoline-3-carboxamide | | |
| 433 | 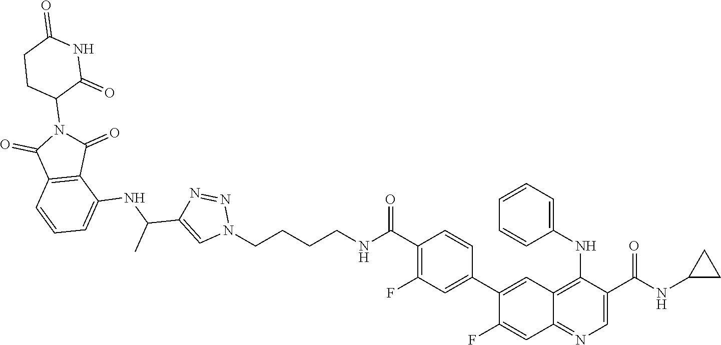 [SMILES: CC(Nc1cccc2c1C(=O)N(C1CCC(=O)NC1=O)C2=O)c1cn(CCCCNC(=O)c2ccc(-c3cc4c(Nc5ccccc5)c(C(=O)NC5CC5)cnc4cc3F)cc2F)nn1] | +++ | |
| | N-cyclopropyl-6-(4-((4-(4-(1-((2-(2,6-dioxopiperidin-3-yl)-1,3- | | |
| | dioxoisoindolin-4-yl)amino)ethyl)-1H-1,2,3-triazol-1- | | |
| | yl)butyl)carbamoyl)-3-fluorophenyl)-7-fluoro-4- | | |
| | (phenylamino)quinoline-3-carboxamide | | |
| 434 | 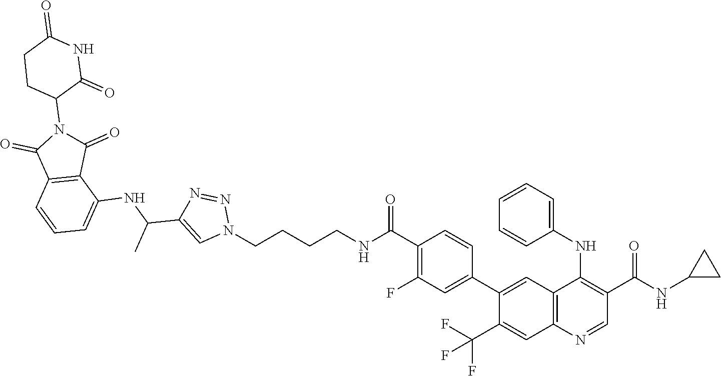 [SMILES: CC(Nc1cccc2c1C(=O)N(C1CCC(=O)NC1=O)C2=O)c1cn(CCCCNC(=O)c2ccc(-c3cc4c(Nc5ccccc5)c(C(=O)NC5CC5)cnc4cc3C(F)(F)F)cc2F)nn1] | +++ | |
| | N-cyclopropyl-6-(4-((4-(4-(1-((2-(2,6-dioxopiperidin-3-yl)-1,3- | | |
| | dioxoisoindolin-4-yl)amino)ethyl)-1H-1,2,3-triazol-1- | | |
| | yl)butyl)carbamoyl)-3-fluorophenyl)-4-(phenylamino)-7- | | |
| | (trifluoromethyl)quinoline-3-carboxamide | | |
| 435 | 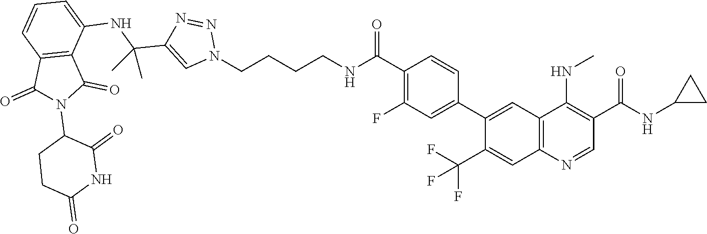 [SMILES: CNc1c(C(=O)NC2CC2)cnc2cc(C(F)(F)F)c(-c3ccc(C(=O)NCCCCn4cc(C(C)(C)Nc5cccc6c5C(=O)N(C5CCC(=O)NC5=O)C6=O)nn4)c(F)c3)cc12] | +++ | |
| | N-cyclopropyl-6-(4-((4-(4-(2-((2-(2,6-dioxopiperidin-3-yl)-1,3- | | |
| | dioxoisoindolin-4-yl)amino)propan-2-yl)-1H-1,2,3-triazol-1- | | |
| | yl)butyl)carbamoyl)-3-fluorophenyl)-4-(methylamino)-7- | | |
| | (trifluoromethyl)quinoline-3-carboxamide | | |
| 436 | 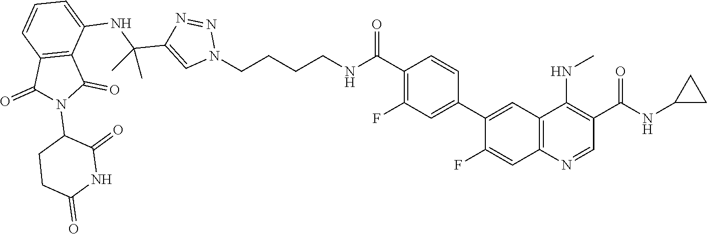 [SMILES: CNc1c(C(=O)NC2CC2)cnc2cc(F)c(-c3ccc(C(=O)NCCCCn4cc(C(C)(C)Nc5cccc6c5C(=O)N(C5CCC(=O)NC5=O)C6=O)nn4)c(F)c3)cc12] | +++ | |
| | N-cyclopropyl-6-(4-((4-(4-(2-((2-(2,6-dioxopiperidin-3-yl)-1,3- | | |
| | dioxoisoindolin-4-yl)amino)propan-2-yl)-1H-1,2,3-triazol-1- | | |
| | yl)butyl)carbamoyl)-3-fluorophenyl)-7-fluoro-4- | | |
| | (methylamino)quinoline-3-carboxamide | | |
| 437 | 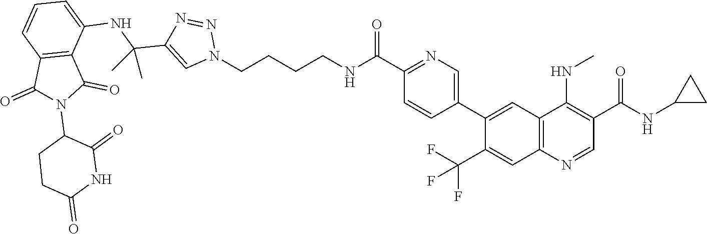 [SMILES: CNc1c(C(=O)NC2CC2)cnc2cc(C(F)(F)F)c(-c3ccc(C(=O)NCCCCn4cc(C(C)(C)Nc5cccc6c5C(=O)N(C5CCC(=O)NC5=O)C6=O)nn4)nc3)cc12] | +++ | |
| | N-cyclopropyl-6-(6-((4-(4-(2-((2-(2,6-dioxopiperidin-3-yl)-1,3- | | |
| | dioxoisoindolin-4-yl)amino)propan-2-yl)-1H-1,2,3-triazol-1- | | |
| | yl)butyl)carbamoyl)pyridin-3-yl)-4-(methylamino)-7- | | |
| | (trifluoromethyl)quinoline-3-carboxamide | | |
| 439 | 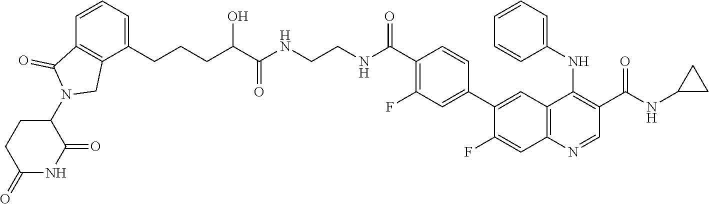 [SMILES: O=C1CCC(N2Cc3c(CCCC(O)C(=O)NCCNC(=O)c4ccc(-c5cc6c(Nc7ccccc7)c(C(=O)NC7CC7)cnc6cc5F)cc4F)cccc3C2=O)C(=O)N1] | +++ | |
| | N-cyclopropyl-6-(4-((2-(5-(2-(2,6-dioxopiperidin-3-yl)-1-oxoisoindolin- | | |
| | 4-yl)-2-hydroxypentanamido)ethyl)carbamoyl)-3-fluorophenyl)-7-fluoro- | | |
| | 4-(phenylamino)quinoline-3-carboxamide | | |
| 440 | 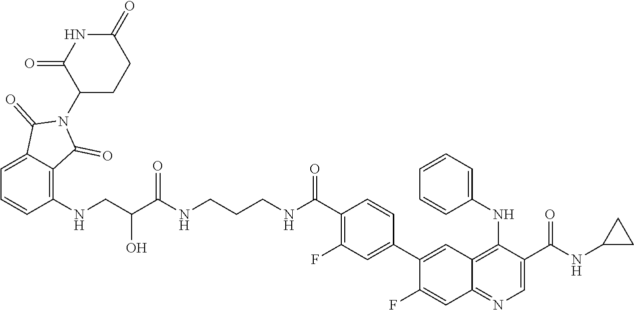 [SMILES: O=C1CCC(N2C(=O)c3cccc(NCC(O)C(=O)NCCCNC(=O)c4ccc(-c5cc6c(Nc7ccccc7)c(C(=O)NC7CC7)cnc6cc5F)cc4F)c3C2=O)C(=O)N1] | +++ | |
| | N-cyclopropyl-6-(4-((3-(3-((2-(2,6-dioxopiperidin-3-yl)-1,3- | | |
| | dioxoisoindolin-4-yl)amino)-2-hydroxypropanamido)propyl)carbamoyl)- | | |
| | 3-fluorophenyl)-7-fluoro-4-(phenylamino)quinoline-3-carboxamide | | |
| 441 | 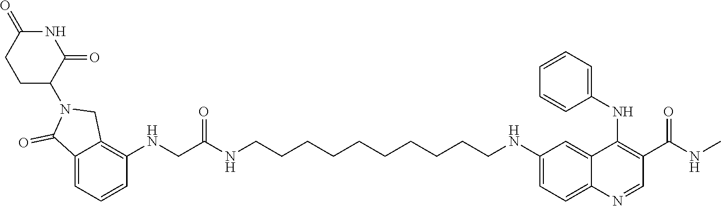 [SMILES: CNC(=O)c1cnc2ccc(NCCCCCCCCCCNC(=O)CNc3cccc4c3CN(C3CCC(=O)NC3=O)C4=O)cc2c1Nc1ccccc1] | +++ | |
| | 6-((10-(2-((2-(2,6-dioxopiperidin-3-yl)-1-oxoisoindolin-4- | | |
| | yl)amino)acetamido)decyl)amino)-N-methyl-4-(phenylamino)quinoline- | | |
| | 3-carboxamide | | |
| 442 | 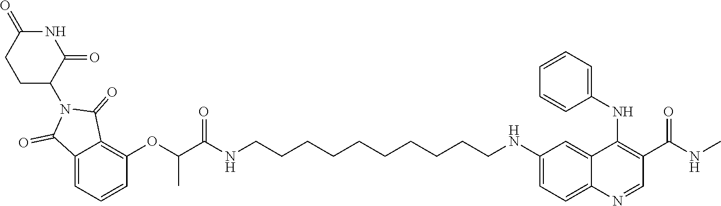 [SMILES: CNC(=O)c1cnc2ccc(NCCCCCCCCCCNC(=O)C(C)Oc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)oxy)propanamido)decyl)amino)-N-methyl-4-(phenylamino)quinoline- | | |
| | 3-carboxamide | | |
| 443 | 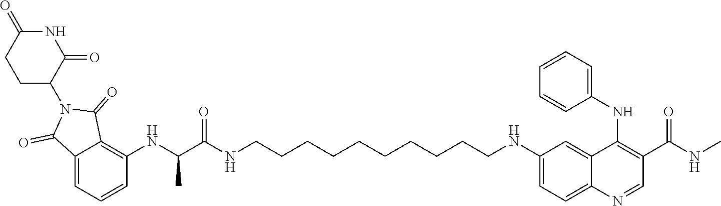 [SMILES: CNC(=O)c1cnc2ccc(NCCCCCCCCCCNC(=O)[C@@H](C)Nc3cccc4c3C(=O)N(C3CCC(=O)NC3=O)C4=O)cc2c1Nc1ccccc1] | +++ | |
| | 6-((10-((2R)-2-((2-(2,6-dioxopiperidin-3-yl)-1,3-dioxoisoindolin-4- | | |
| | yl)amino)propanamido)decyl)amino)-N-methyl-4- | | |
| | (phenylamino)quinoline-3-carboxamide | | |
| 444 | 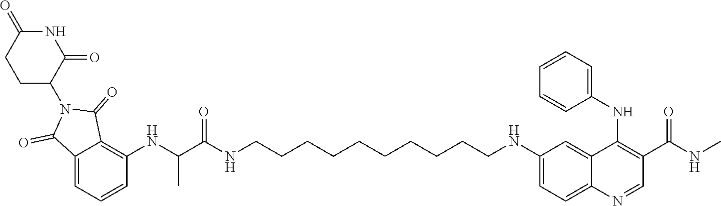 [SMILES: CNC(=O)c1cnc2ccc(NCCCCCCCCCCNC(=O)C(C)Nc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)amino)propanamido)decyl)amino)-N-methyl-4- | | |
| | (phenylamino)quinoline-3-carboxamide | | |
| 445 | 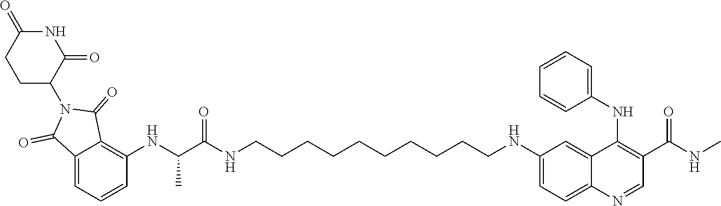 [SMILES: CNC(=O)c1cnc2ccc(NCCCCCCCCCCNC(=O)[C@H](C)Nc3cccc4c3C(=O)N(C3CCC(=O)NC3=O)C4=O)cc2c1Nc1ccccc1] | +++ | |
| | 6-((10-((2S)-2((2-(2,6-dioxopiperidin-3-yl)-1,3-dioxoisoindolin-4- | | |
| | yl)amino)propanamido)decyl)amino)-N-methyl-4- | | |
| | (phenylamino)quinoline-3-carboxamide | | |
| 446 | 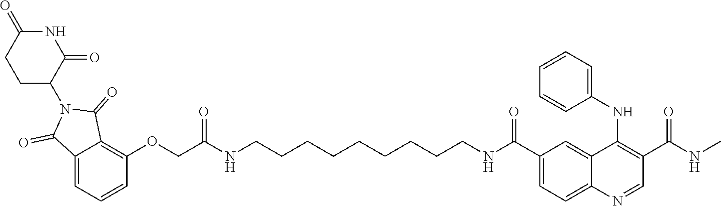 [SMILES: CNC(=O)c1cnc2ccc(C(=O)NCCCCCCCCCNC(=O)COc3cccc4c3C(=O)N(C3CCC(=O)NC3=O)C4=O)cc2c1Nc1ccccc1] | +++ | |
| | N6-(9-(2-((2-(2,6-dioxopiperidin-3-yl)-1,3-dioxoisoindolin-4- | | |
| | yl)oxy)acetamido)nonyl)-N3-methyl-4-(phenylamino)quinoline-3,6- | | |
| | dicarboxamide | | |
| 447 | 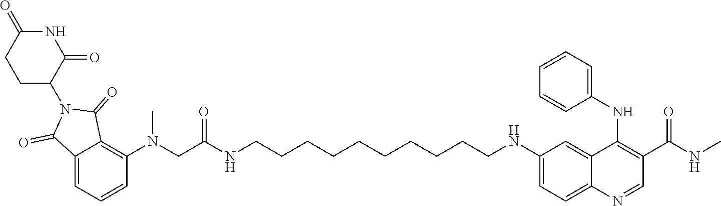 [SMILES: CNC(=O)c1cnc2ccc(NCCCCCCCCCCNC(=O)CN(C)c3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)(methyl)amino)acetamido)decyl)amino)-N-methyl-4- | | |
| | (phenylamino)quinoline-3-carboxamide | | |
| 448 | 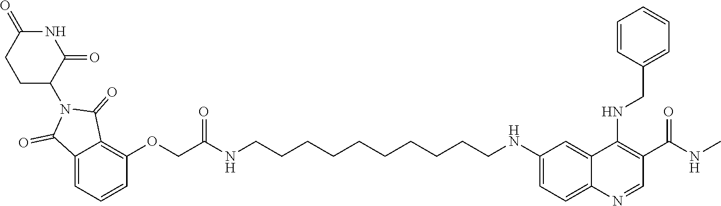 [SMILES: CNC(=O)c1cnc2ccc(NCCCCCCCCCCNC(=O)COc3cccc4c3C(=O)N(C3CCC(=O)NC3=O)C4=O)cc2c1NCc1ccccc1] | +++ | |
| | 4-(benzylamino)-6-((10-(2-((2-(2,6-dioxopiperidin-3-yl)-1,3- | | |
| | dioxoisoindolin-4-yl)oxy)acetamido)decyl)amino)-N-methylquinoline-3- | | |
| | carboxamide | | |
| 449 | 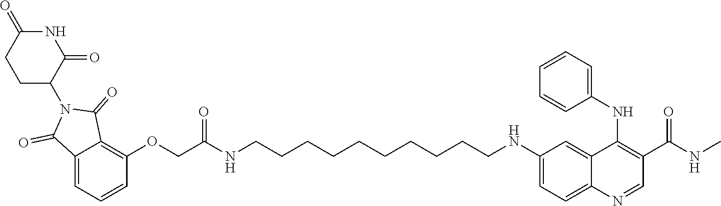 [SMILES: CNC(=O)c1cnc2ccc(NCCCCCCCCCCNC(=O)COc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)oxy)acetamido)decyl)amino)-N-methyl-4-(phenylamino)quinoline-3- | | |
| | carboxamide | | |
| 450 | 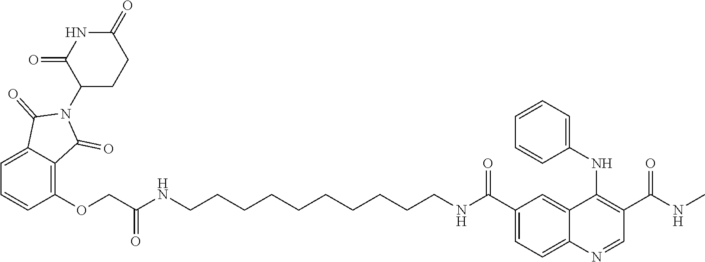 [SMILES: CNC(=O)c1cnc2ccc(C(=O)NCCCCCCCCCCNC(=O)COc3cccc4c3C(=O)N(C3CCC(=O)NC3=O)C4=O)cc2c1Nc1ccccc1] | +++ | |
| | N6-(10-(2-((2-(2,6-dioxopiperidin-3-yl)-1,3-dioxoisoindolin-4- | | |
| | yl)oxy)acetamido)decyl)-N3-methyl-4-(phenylamino)quinoline-3,6- | | |
| | dicarboxamide | | |
| 451 | 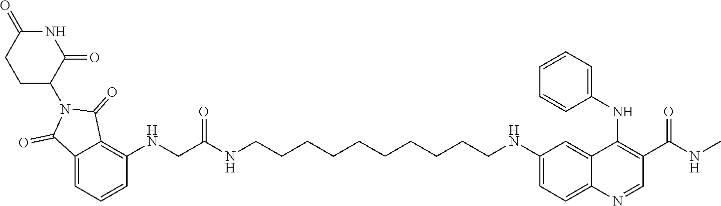 [SMILES: CNC(=O)c1cnc2ccc(NCCCCCCCCCCNC(=O)CNc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)amino)acetamido)decyl)amino)-N-methyl-4-(phenylamino)quinoline- | | |
| | 3-carboxamide | | |
| 452 | 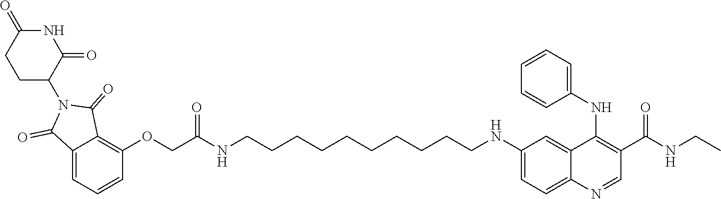 [SMILES: CCNC(=O)c1cnc2ccc(NCCCCCCCCCCNC(=O)COc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)oxy)acetamido)decyl)amino)-N-ethyl-4-(phenylamino)quinoline-3- | | |
| | carboxamide | | |
| 453 | 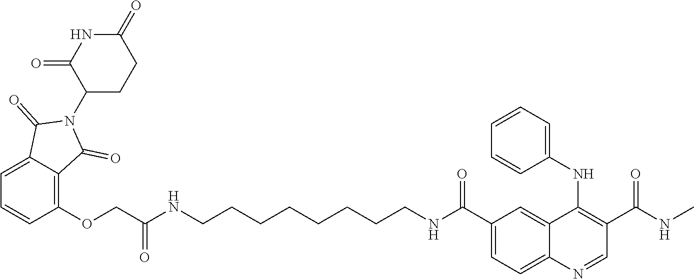 [SMILES: CNC(=O)c1cnc2ccc(C(=O)NCCCCCCCCNC(=O)COc3cccc4c3C(=O)N(C3CCC(=O)NC3=O)C4=O)cc2c1Nc1ccccc1] | +++ | |
| | N6-(8-(2-((2-(2,6-dioxopiperidin-3-yl)-1,3-dioxoisoindolin-4- | | |
| | yl)oxy)acetamido)octyl)-N3-methyl-4-(phenylamino)quinoline-3,6- | | |
| | dicarboxamide | | |
| 454 | 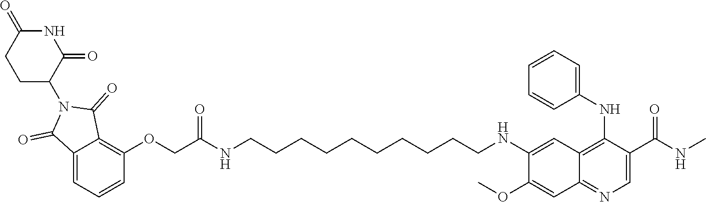 [SMILES: CNC(=O)c1cnc2cc(OC)c(NCCCCCCCCCCNC(=O)COc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)oxy)acetamido)decyl)amino)-7-methoxy-N-methyl-4- | | |
| | (phenylamino)quinoline-3-carboxamide | | |
| 455 | 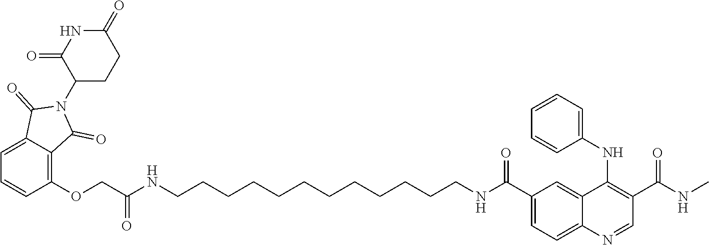 [SMILES: CNC(=O)c1cnc2ccc(C(=O)NCCCCCCCCCCCCNC(=O)COc3cccc4c3C(=O)N(C3CCC(=O)NC3=O)C4=O)cc2c1Nc1ccccc1] | +++ | |
| | N6-(12-(2-((2-(2,6-dioxopiperidin-3-yl)-1,3-dioxoisoindolin-4- | | |
| | yl)oxy)acetamido)dodecyl)-N3-methyl-4-(phenylamino)quinoline-3,6- | | |
| | dicarboxamide | | |
| 456 | 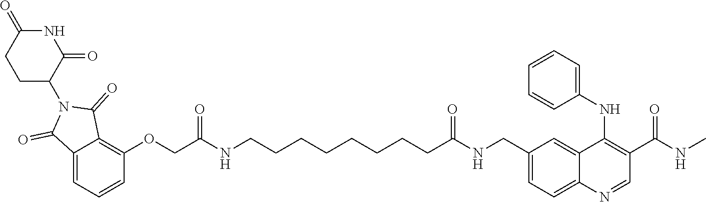 [SMILES: CNC(=O)c1cnc2ccc(CNC(=O)CCCCCCCCNC(=O)COc3cccc4c3C(=O)N(C3CCC(=O)NC3=O)C4=O)cc2c1Nc1ccccc1] | +++ | |
| | 6-((9-(2-((2-(2,6-dioxopiperidin-3-yl)-1,3-dioxoisoindolin-4- | | |
| | yl)oxy)acetamido)nonanamido)methyl)-N-methyl-4- | | |
| | (phenylamino)quinoline-3-carboxamide | | |
| 457 | 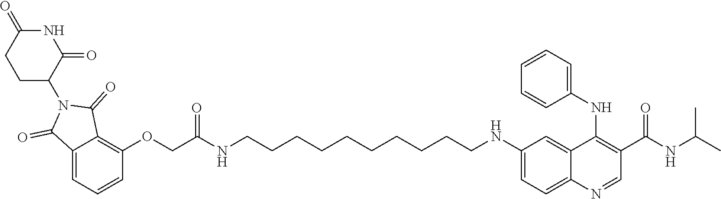 [SMILES: CC(C)NC(=O)c1cnc2ccc(NCCCCCCCCCCNC(=O)COc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)oxy)acetamido)decyl)amino)-N-isopropyl-4-(phenylamino)quinoline- | | |
| | 3-carboxamide | | |
| 458 | 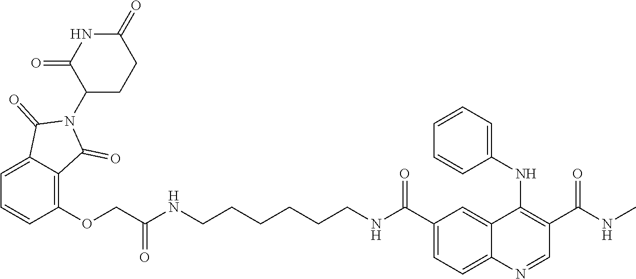 [SMILES: CNC(=O)c1cnc2ccc(C(=O)NCCCCCCNC(=O)COc3cccc4c3C(=O)N(C3CCC(=O)NC3=O)C4=O)cc2c1Nc1ccccc1] | +++ | |
| | N6-(6-(2-((2-(2,6-dioxopiperidin-3-yl)-1,3-dioxoisoindolin-4- | | |
| | yl)oxy)acetamido)hexyl)-N3-methyl-4-(phenylamino)quinoline-3,6- | | |
| | dicarboxamide | | |
| 459 | 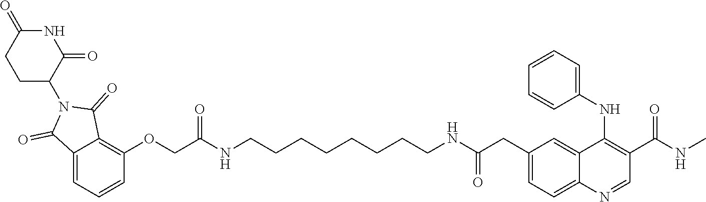 [SMILES: CNC(=O)c1cnc2ccc(CC(=O)NCCCCCCCCNC(=O)COc3cccc4c3C(=O)N(C3CCC(=O)NC3=O)C4=O)cc2c1Nc1ccccc1] | +++ | |
| | 6-(2-((8-(2-((2-(2,6-dioxopiperidin-3-yl)-1,3-dioxoisoindolin-4- | | |
| | yl)oxy)acetamido)octyl)amino)-2-oxoethyl)-N-methyl-4- | | |
| | (phenylamino)quinoline-3-carboxamide | | |
| 460 | 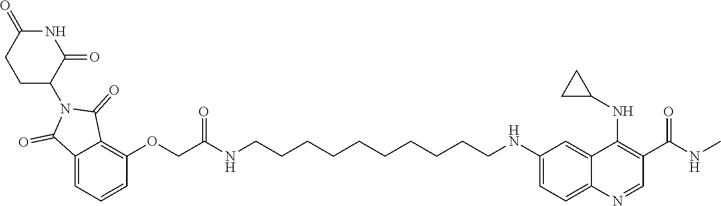 [SMILES: CNC(=O)c1cnc2ccc(NCCCCCCCCCCNC(=O)COc3cccc4c3C(=O)N(C3CCC(=O)NC3=O)C4=O)cc2c1NC1CC1] | | +++ |
| | 4-(cyclopropylamino)-6-((10-(2-((2-(2,6-dioxopiperidin-3-yl)-1,3- | | |
| | dioxoisoindolin-4-yl)oxy)acetamido)decyl)amino)-N-methylquinoline-3- | | |
| | carboxamide | | |
| 464 | 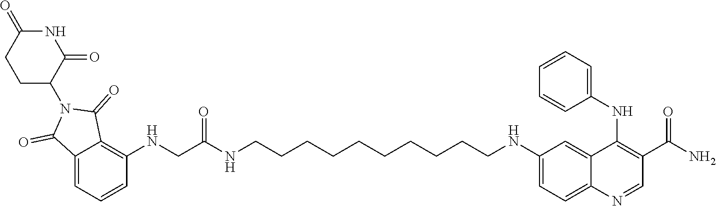 [SMILES: NC(=O)c1cnc2ccc(NCCCCCCCCCCNC(=O)CNc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)amino)acetamido)decyl)amino)-4-(phenylamino)quinoline-3- | | |
| | carboxamide | | |
| 465 | 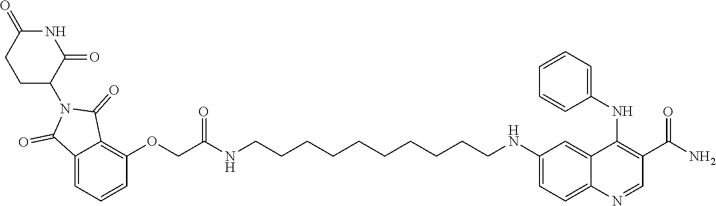 [SMILES: NC(=O)c1cnc2ccc(NCCCCCCCCCCNC(=O)COc3cccc4c3C(=O)N(C3CCC(=O)NC3=O)C4=O)cc2c1Nc1ccccc1] | +++ | |
| | 6-((10-(2-((2-(2,6-dioxopiperidin-3-yl)-1,3-dioxoisoindolin-4- | | |
| | yl)oxy)acetamido)decyl)amino)-4-(phenylamino)quinoline-3- | | |
| | carboxamide | | |
| 466 | 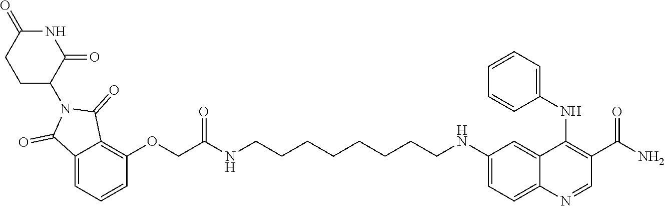 [SMILES: NC(=O)c1cnc2ccc(NCCCCCCCCNC(=O)COc3cccc4c3C(=O)N(C3CCC(=O)NC3=O)C4=O)cc2c1Nc1ccccc1] | +++ | |
| | 6-((8-(2-((2-(2,6-dioxopiperidin-3-yl)-1,3-dioxoisoindolin-4- | | |
| | yl)oxy)acetamido)octyl)amino)-4-(phenylamino)quinoline-3- | | |
| | carboxamide | | |
| 467 | 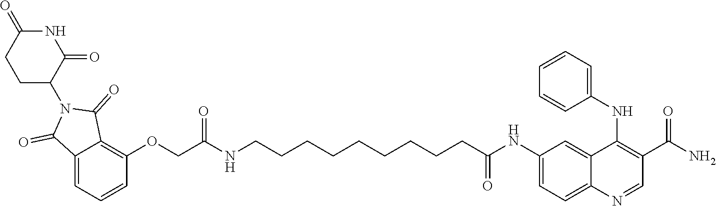 [SMILES: NC(=O)c1cnc2ccc(NC(=O)CCCCCCCCCNC(=O)COc3cccc4c3C(=O)N(C3CCC(=O)NC3=O)C4=O)cc2c1Nc1ccccc1] | +++ | |
| | 6-(10-(2-((2-(2,6-dioxopiperidin-3-yl)-1,3-dioxoisoindolin-4- | | |
| | yl)oxy)acetamido)decanamido)-4-(phenylamino)quinoline-3- | | |
| | carboxamide | | |
| 471 | 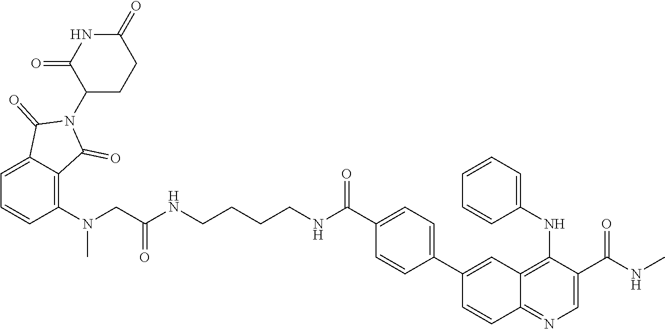 [SMILES: CNC(=O)c1cnc2ccc(-c3ccc(C(=O)NCCCCNC(=O)CN(C)c4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)(methyl)amino)acetamido)butyl)carbamoyl)phenyl)-N-methyl-4- | | |
| | (phenylamino)quinoline-3-carboxamide | | |
| 472 | 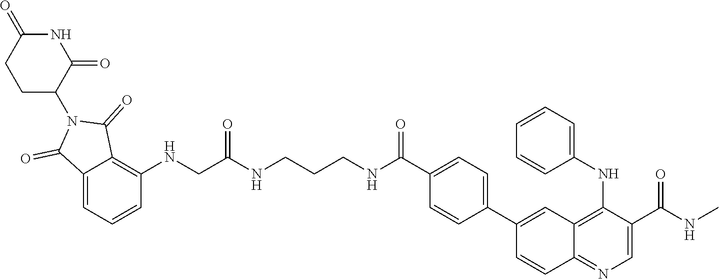 [SMILES: CNC(=O)c1cnc2ccc(-c3ccc(C(=O)NCCCNC(=O)CNc4cccc5c4C(=O)N(C4CCC(=O)NC4=O)C5=O)cc3)cc2c1Nc1ccccc1] | +++ | |
| | 6-(4-((3-(2-((2-(2,6-dioxopiperidin-3-yl)-1,3-dioxoisoindolin-4- | | |
| | yl)amino)acetamido)propyl)carbamoyl)phenyl)-N-methyl-4- | | |
| | (phenylamino)quinoline-3-carboxamide | | |
| 473 | 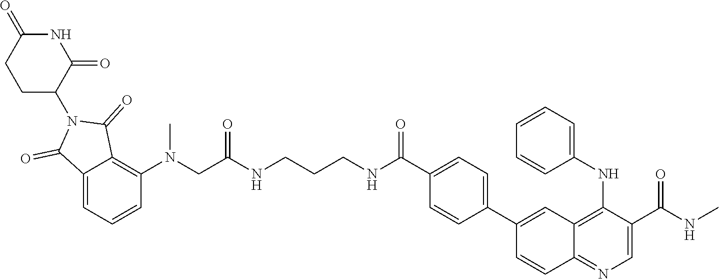 [SMILES: CNC(=O)c1cnc2ccc(-c3ccc(C(=O)NCCCNC(=O)CN(C)c4cccc5c4C(=O)N(C4CCC(=O)NC4=O)C5=O)cc3)cc2c1Nc1ccccc1] | +++ | |
| | 6-(4-((3-(2-((2-(2,6-dioxopiperidin-3-yl)-1,3-dioxoisoindolin-4- | | |
| | yl)(methyl)amino)acetamido)propyl)carbamoyl)phenyl)-N-methyl-4- | | |
| | (phenylamino)quinoline-3-carboxamide | | |
| 474 | 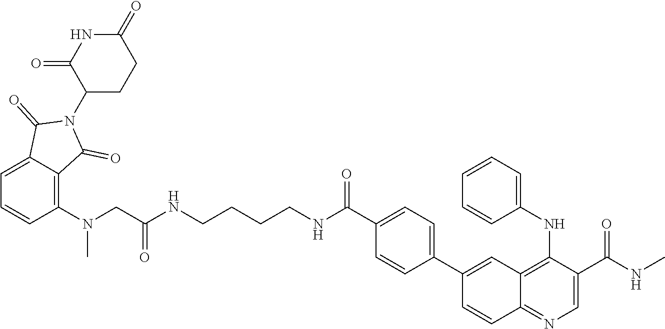 [SMILES: CNC(=O)c1cnc2ccc(-c3ccc(C(=O)NCCCCNC(=O)CN(C)c4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)(methyl)amino)acetamido)butyl)carbamoyl)phenyl)-N-methyl-4- | | |
| | (phenylamino)quinoline-3-carboxamide | | |
| 475 | 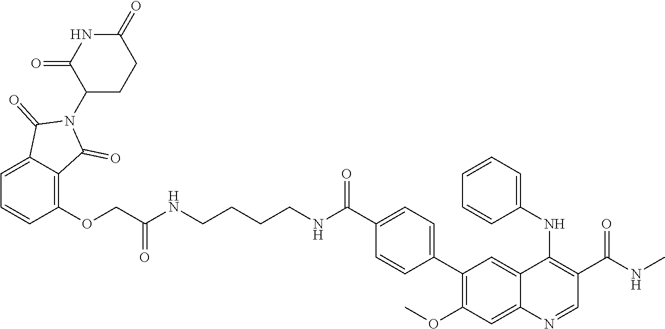 [SMILES: CNC(=O)c1cnc2cc(OC)c(-c3ccc(C(=O)NCCCCNC(=O)COc4cccc5c4C(=O)N(C4CCC(=O)NC4=O)C5=O)cc3)cc2c1Nc1ccccc1] | +++ | |
| | 6-(4-((4-(2-((2-(2,6-dioxopiperidin-3-yl)-1,3-dioxoisoindolin-4- | | |
| | yl)oxy)acetamido)butyl)carbamoyl)phenyl)-7-methoxy-N-methyl-4- | | |
| | (phenylamino)quinoline-3-carboxamide | | |

In the above table +++ is <100 nM and ++ is <1000 nM

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teachings of this invention that certain changes and modifications may be made thereto without departing from the spirit or scope of the invention as defined in the appended claims.

We claim:

1. A compound selected from: 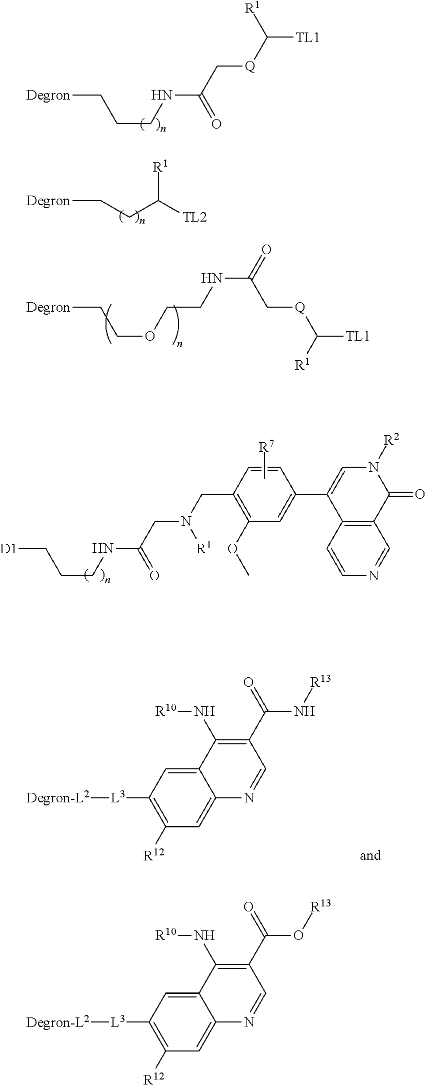
or a pharmaceutically acceptable salt thereof;
wherein:
Degron is selected from: 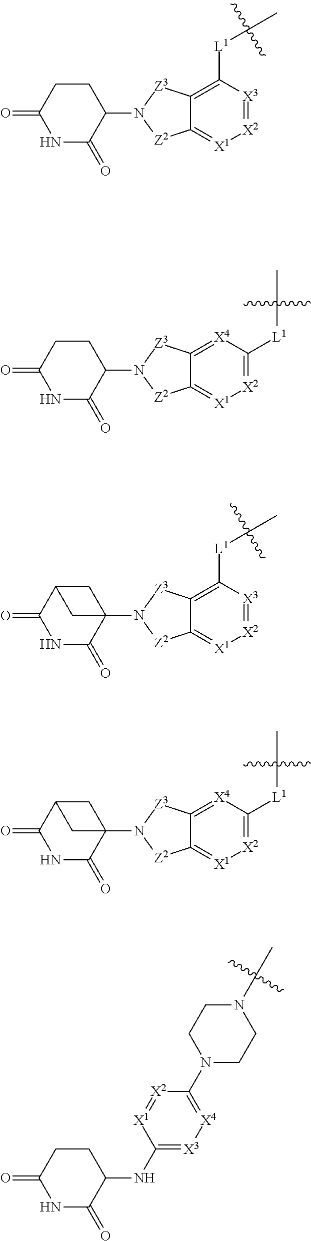 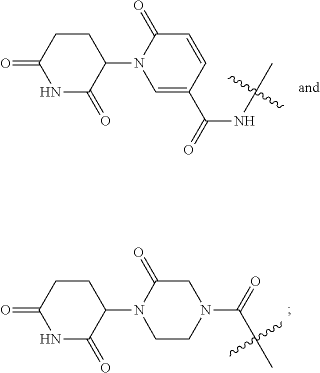 [SMILES: *NC(=O)c1ccc(=O)n(C2CCC(=O)NC2=O)c1 ; *C(=O)N1CCN(C2CCC(=O)NC2=O)C(=O)C1]
D1 is selected from: 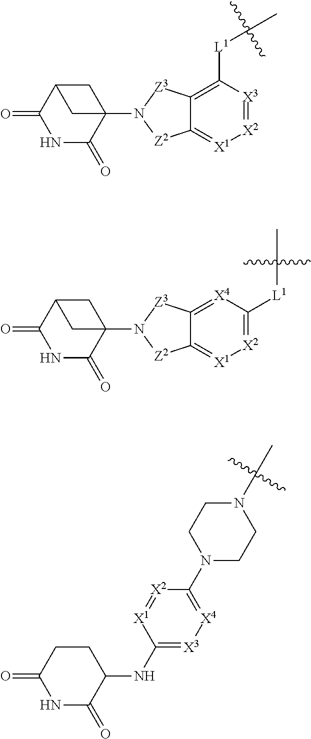 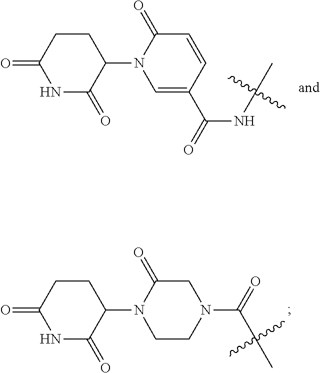 [SMILES: *NC(=O)c1ccc(=O)n(C2CCC(=O)NC2=O)c1 ; *C(=O)N1CCN(C2CCC(=O)NC2=O)C(=O)C1]
TL1 is a moiety that binds to BRD9 selected from 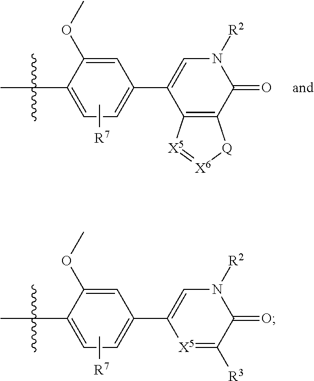
TL2 is a moiety that binds to BRD9 selected from 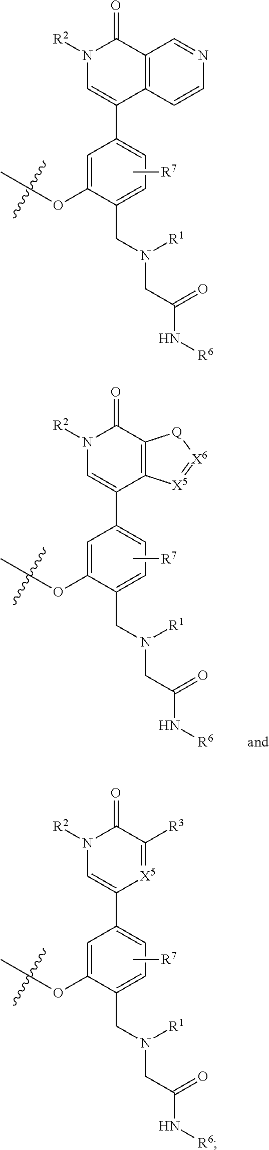
L¹ is selected from: 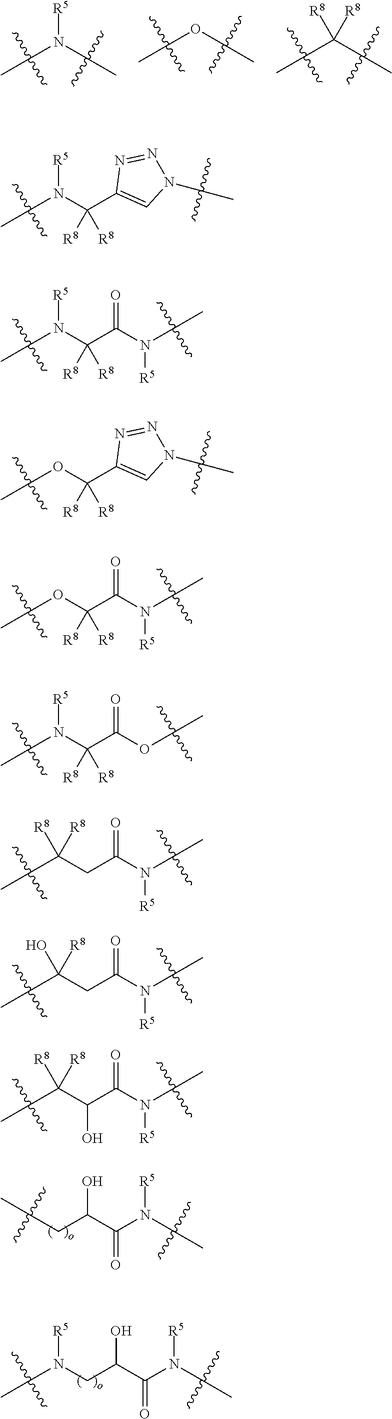 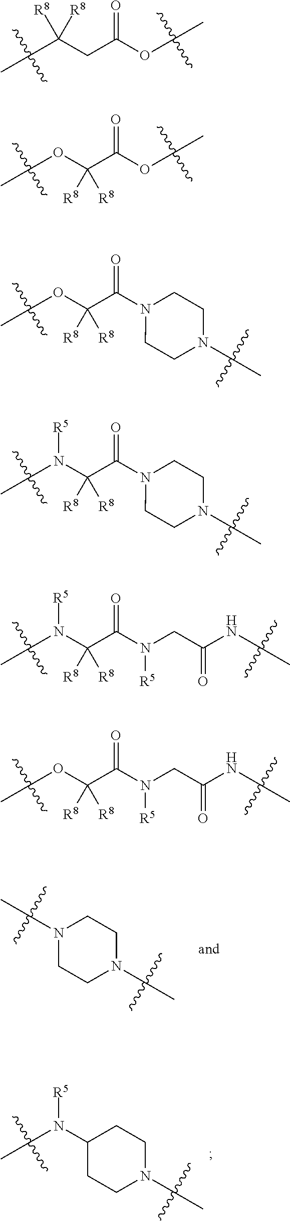
X¹, X², X³, and X⁴ are independently selected from CR⁴ and N, wherein no more than two of X¹, X², X³, and X⁴ may be selected to be N;
X⁵ and X⁶ are independently selected from CR⁴ and N;
Z² and Z³ are selected from —CH₂— and —C(O)— wherein at least one of Z² and Z³ is —C(O)—;
n is 0, 1, 2, 3, 4, 5, 6, 7, 8, 9, or 10;
o is 1, 2, 3, or 4;
each Q is independently 0, S, or NR⁵;
R¹ is hydrogen or C₁-C₆ alkyl;
R², R³, and R⁶ are independently selected from hydrogen and C₁-C₆alkyl;
each R⁴ is independently selected from hydrogen, halogen, hydroxyl, C₁-C₆alkyl, C₁-C₆alkoxy, and C₁-C₆haloalkyl;
each R⁵ is independently hydrogen, C₁-C₆alkyl, or —C(O)alkyl;
R⁷ is selected from halogen, hydrogen, C₁-C₆alkyl, C₁-C₆alkoxy, and C₁-C₆haloalkyl;
each R⁸ is independently selected from hydrogen, C₁-C₆alkyl, and C₁-C₆haloalkyl; or two R⁸ groups together with the carbon to which they are attached form a cyclopropyl group;
L² is selected from: bond 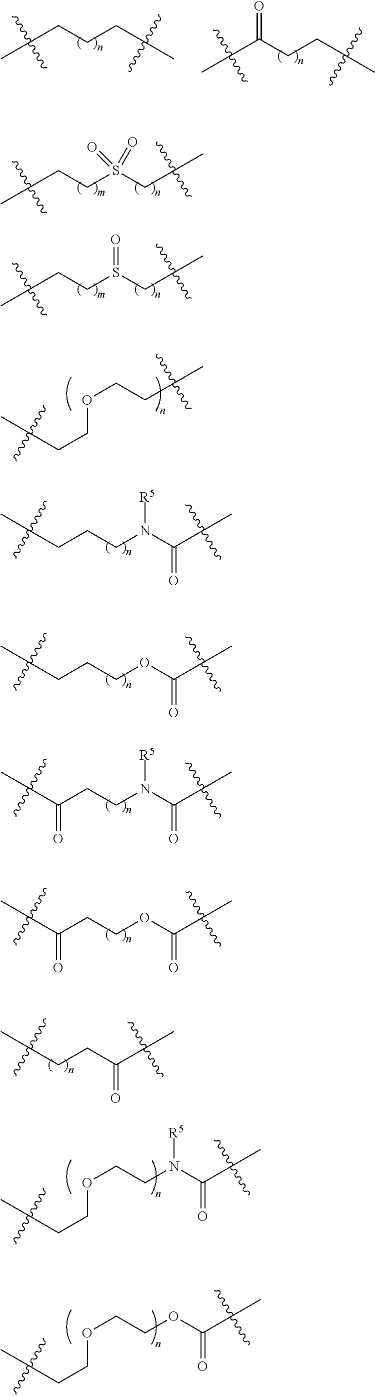 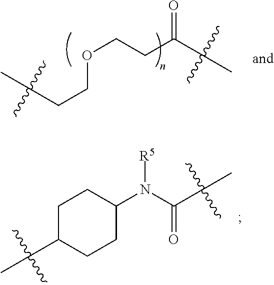
L³ is selected from bond, aryl, heterocycle, heteroaryl, 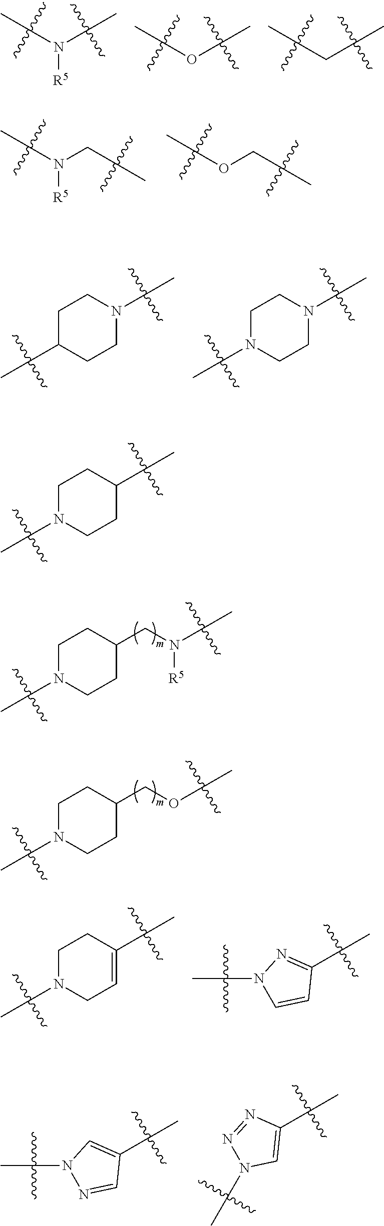 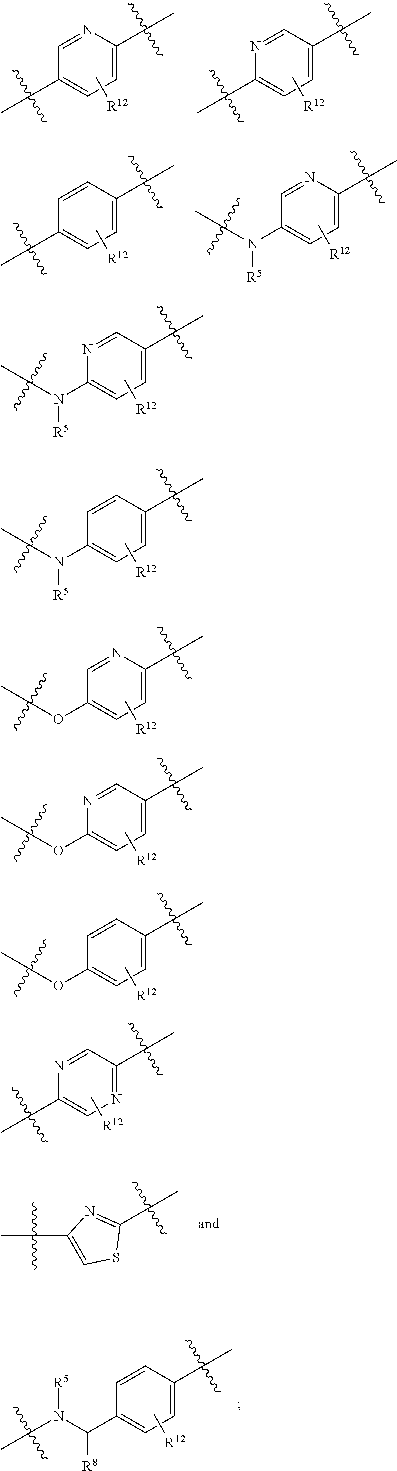
m is 0, 1, 2, 3, 4, 5, 6, 7, 8, 9, or 10;
R¹⁰ is selected from C₁-C₆alkyl, cycloalkyl, heterocycle, heteroaryl, —C₁-C₆alkyl-aryl, and aryl; each of which R¹⁰ group is optionally substituted with 1, 2, 3, or 4 substituents independently selected from R¹¹;
R¹¹ is selected from hydrogen, halogen, —NR¹R¹⁴, —OR¹¹, C₁-C₆alkyl, C₁-C₆haloalkyl, —SO₂NR¹R¹⁴, —SO₂OR¹⁴, —SONR¹R¹⁴, and —S(O)OR¹⁴;
each R¹² is independently selected from hydrogen, halogen, C₁-C₆alkyl, C₁-C₆alkoxy, and C₁-C₆haloalkyl;
R¹³ is selected from hydrogen, C₁-C₆alkyl, cycloalkyl, and heterocycle; each of which cycloalkyl and heterocycle is optionally substituted with 1, 2, 3, or 4 substituents independently selected from R¹¹; and
each instance of R¹⁴ is independently selected from hydrogen, C₁-C₆alkyl, C(O)alkyl, and C(O)NR¹R¹.

2. The compound of claim 1, wherein TL1 is:

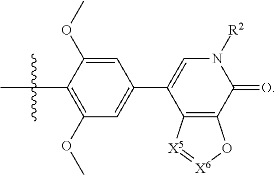

3. The compound of claim 1, wherein TL1 is:

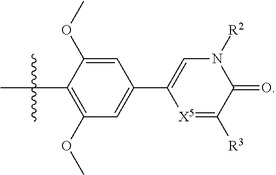

4. The compound of claim 1, wherein the compound is selected from 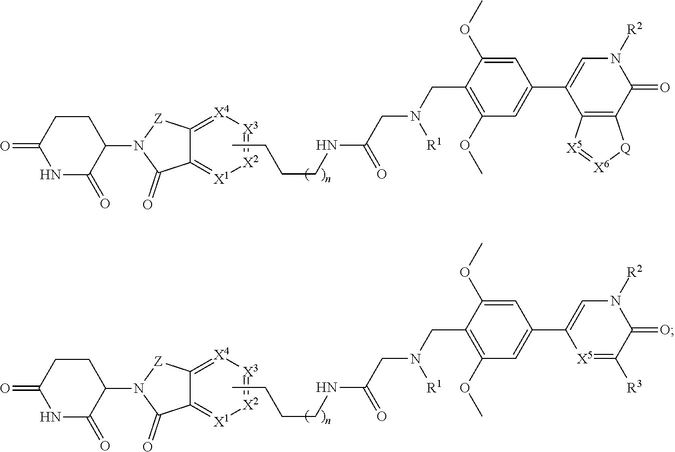
or a pharmaceutically acceptable salt thereof;
wherein
Z is CH₂ or C(O).

5. The compound of claim 1, wherein the compound is selected from 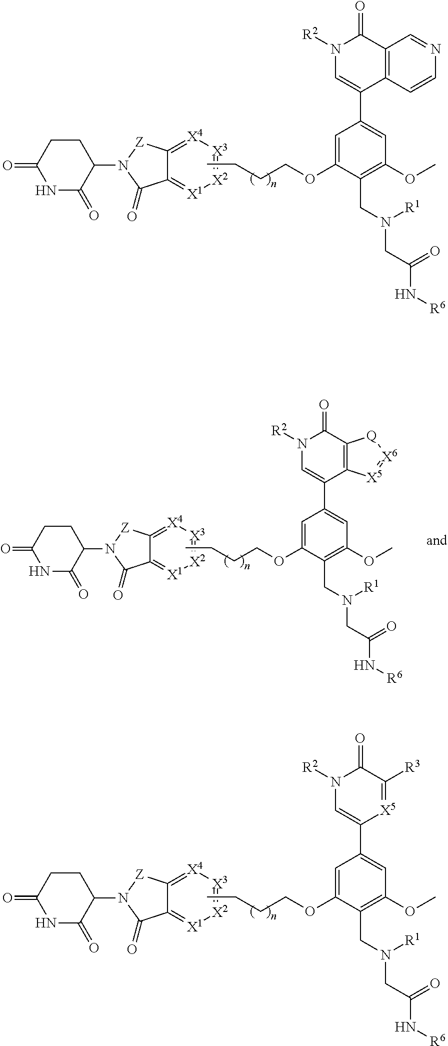
or a pharmaceutically acceptable salt thereof;
wherein
Z is CH₂ or C(O).

6. The compound of claim 1, wherein the compound is: 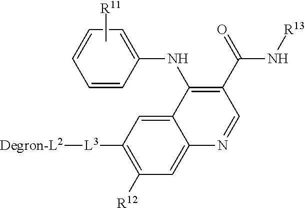
or a pharmaceutically acceptable salt thereof.

7. The compound of claim 1, wherein the compound is selected from: 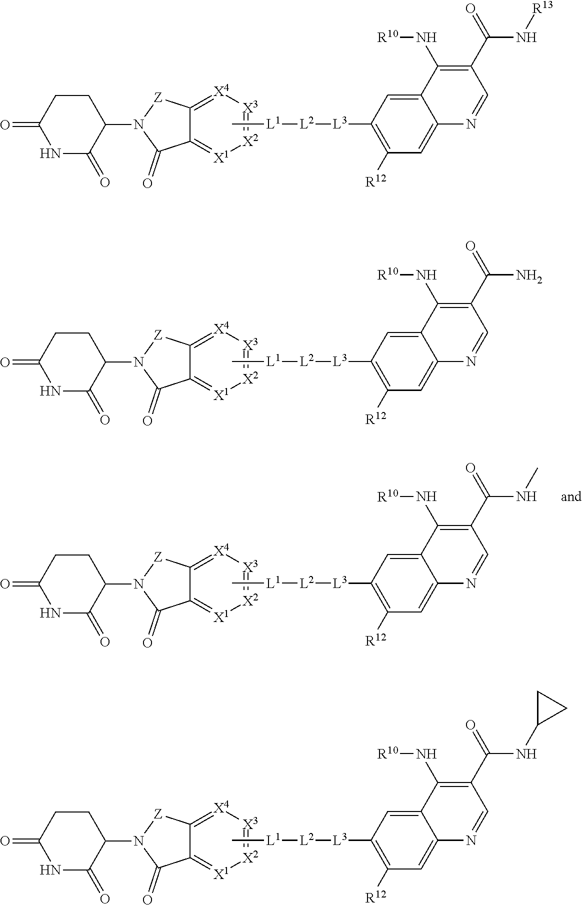
or a pharmaceutically acceptable salt thereof;
wherein
Z is CH₂ or C(O).

8. The compound of claim 1, wherein L²-L³ is selected from:

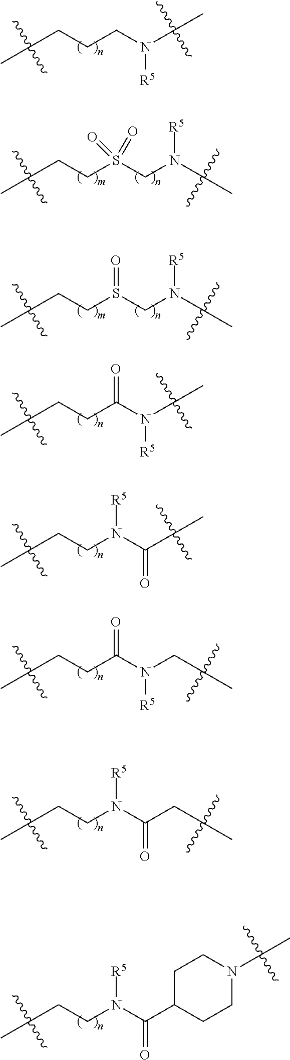

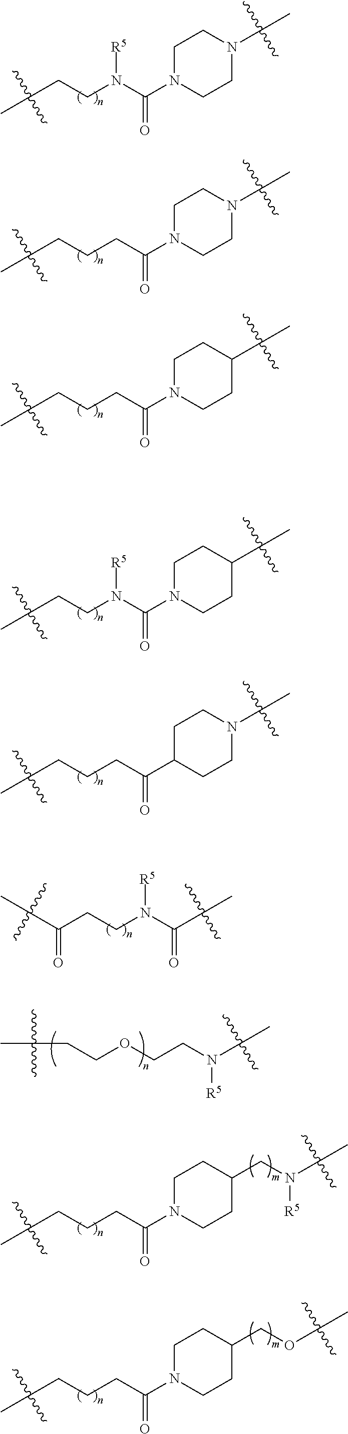

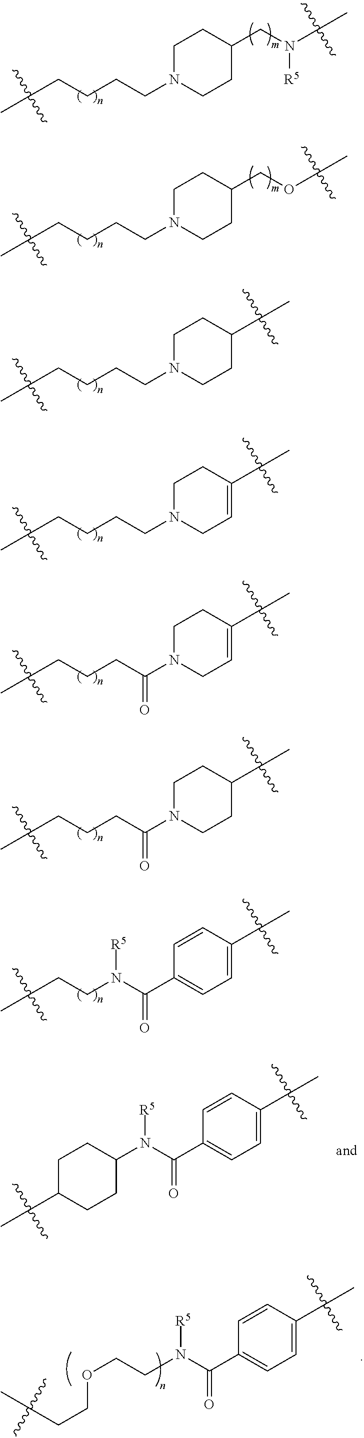

9. The compound of claim 1, wherein the compound is selected from: 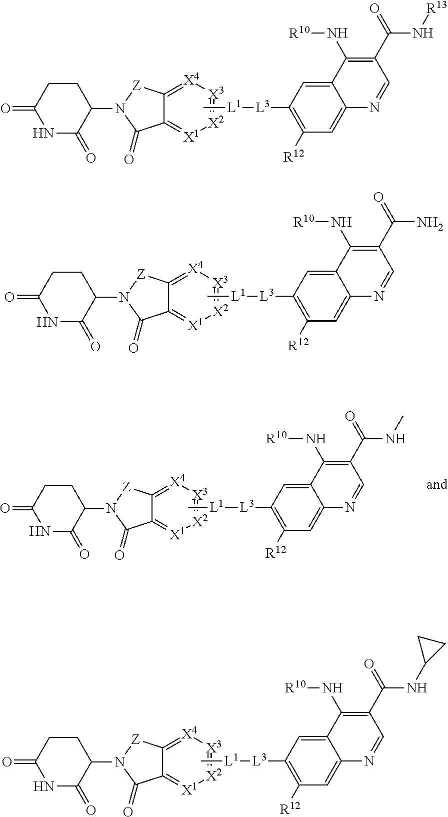
or a pharmaceutically acceptable salt thereof;
wherein
Z is CH₂ or C(O).

10. The compound of claim 1, wherein Z² and Z are C(O).

11. The compound of claim 1, wherein R¹ is hydrogen.

12. The compound of claim 1, wherein R⁵ is hydrogen.

13. The compound of claim 1, wherein R⁵ is C₁-C₆alkyl.

14. The compound of claim 1, wherein n is 0, 1, 2, or 3.

15. The compound of claim 1, wherein Degron or D1 are selected from: 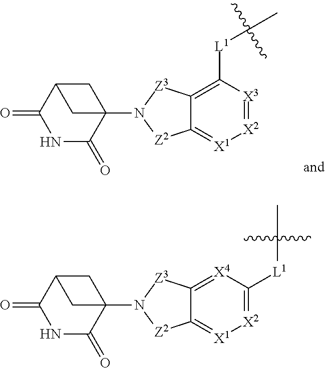
or a pharmaceutically acceptable salt thereof.

16. The compound of claim 1, wherein the compound is selected from Table 1 or a pharmaceutically acceptable salt thereof.

17. The compound of claim 1, wherein the compound is selected from Table 2 or a pharmaceutically acceptable salt thereof.

18. A pharmaceutical composition comprising a compound of claim 1 or a pharmaceutically acceptable salt thereof, and a pharmaceutically acceptable carrier.

19. A method for treating a BRD9 or MTH1 mediated disorder comprising administering to a subject in need thereof a therapeutically effective amount of a compound of claim 1 or a pharmaceutically acceptable salt thereof.

20. The method of claim 19, wherein the subject is a human.
